(19) United States
(12) Patent Application Publication
(10) Pub. No.: US 2024/0368173 A1
(43) Pub. Date: Nov. 7, 2024

(54) TRICYCLIC GPR65 MODULATORS

(71) Applicant: PATHIOS THERAPEUTICS LIMITED, ABINGDON UK (GB)

(72) Inventors: Tom MCCARTHY, Milton, Abingdon UK (GB); Alan NAYLOR, Milton, Abingdon UK (GB); David MILLER, Milton, Abingdon UK (GB); Gavin MILNE, Milton, Abingdon UK (GB); Maria IEVA, Milton, Abingdon UK (GB); Tobias MOECHEL, Milton, Abingdon UK (GB); Yannick ESVAN, Milton, Abingdon UK (GB); John FEUTRILL, Milton, Abingdon UK (GB)

(21) Appl. No.: 18/699,825

(22) PCT Filed: Oct. 17, 2022
(86) PCT No.: PCT/GB2022/052644

§ 371 Date: Apr. 9, 2024

(30) Foreign Application Priority Data
Oct. 18, 2021 (GB) 2114866.3
Dec. 3, 2021 (GB) 2117501.3
Jun. 13, 2022 (GB) 2208626.8
Aug. 8, 2022 (GB) 2211546.3
Sep. 21, 2022 (GB) 2213798.8

(51) Int. Cl. C07D 487/18; A61K 31/439; A61K 31/5025; A61K 31/506; A61K 31/5377; C07D 471/18; C07D 519/00

(52) U.S. Cl. CPC C07D 487/18; A61K 31/439; A61K 31/5025; A61K 31/506; A61K 31/5377; C07D 471/18; C07D 519/00

(57) ABSTRACT

One aspect of the invention relates to a compound of formula (I), or a pharmaceutically acceptable salt or solvate thereof, (I) wherein: ring B is: a monocyclic aromatic group; or a monocyclic or bicyclic heteroaromatic group, each of which is optionally substituted by halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups; m is an integer from 0 to 3; p and q are each independently 0 to 3; Z is CR₁₂; Y is CR₁₀R_{10′}, wherein R₁₀ and R_{10′}, are each independently selected from H, F, alkyl, and haloalkyl; Rₐ and R_{b} are each independently selected from H and alkyl; R₆ is selected from H, alkyl, cycloalkyl and hydroxyalkyl; R₁₂ is selected from H, alkyl, haloalkyl, halo, OH and O-alkyl; and R₁₃, R_{13′}, R₁₄, R_{14′}, R₁₅, R_{15′}, and R₁₆ are each independently selected from H, alkyl, and alkoxyalkyl. Further aspects of the invention relate to compounds of formula (I) for use as a medicament, particularly in the field of immuno-oncology, immunology, and related applications.

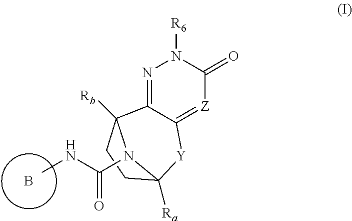

The present invention relates to compounds that are capable of modulating GPR65. The compounds have potential therapeutic applications in the treatment of a variety of disorders including proliferative and immune disorders.

### BACKGROUND TO THE INVENTION

GPR65 is a Gs-coupled G protein-coupled receptor (GPCR) that is primarily expressed in immune cells and is activated by acidic extracellular pH to cause increases in cytoplasmic cyclic adenosine monophosphate (cAMP) (Wang, 2004). It has long been known that tumours typically undergo a switch in cellular metabolism from oxidative phosphorylation to aerobic glycolysis, which in turn results in an acidic extracellular microenvironment (Damaghi, 2013). Recently, it has been shown that this acidic microenvironment causes GPR65 activation in tumour-associated macrophages, resulting in an increase in cytoplasmic cAMP leading to transcription of the inducible cAMP early repressor (ICER). This, in turn, suppresses the secretion of tumour necrosis factor alpha (TNFα) to bias the macrophages toward an anti-inflammatory, tumour-permissive phenotype (Bohn, 2018). This GPR65-dependent pathway therefore appears to represent a mechanism by which tumours exploit their acidic microenvironment to evade detection by the immune system.

Autoimmune diseases are also often associated with an acidic local microenvironment (for instance, an inflamed joint). Recent studies also suggest that GPR65 acts through ICER in CD4+ T cells, to suppress IL-2 and hence bias cells toward an inflammatory Th17 phenotype, which is associated with increased pathogenicity in the context of autoimmune disease (Korn, 2009). Supporting this is the recent finding that ICER is required for Th17 differentiation (Yoshida, 2016) as well as that agonism of GPR65 leads to an increase in Th17 differentiation (Hernandez, 2018). Indeed, mutations in the GPR65 locus are associated with several autoimmune diseases, such as multiple sclerosis, ankylosing spondylitis, inflammatory bowel disease, and Crohn's disease (Gaublomme, 2015). One recent study found that mice with CD4+ T cells lacking GPR65 were protected from developing the disease autoimmune encephalomyelitis (EAE) (Gaublomme, 2015).

Thus, GPR65 appears to act through ICER to promote an anti-inflammatory and tumour-permissive phenotype in tumour associated macrophages and an inflammatory Th17 phenotype in CD4+ T cells that is associated with autoimmune disease. GPR65 signalling, therefore, represents an attractive pathway for therapeutic intervention for the treatment of both cancer and autoimmune diseases. There is therefore an ongoing need to develop new small molecule GPR65 modulators.

WO 2021/245427 (Pathios Therapeutics Limited) discloses a series of small molecule GPR65 modulators. The present invention seeks to provide further compounds that are capable of modulating GPR65. As made clear from the above discussion, such compounds have potential therapeutic applications in the treatment of a variety of disorders, including proliferative disorders and immune disorders, as well as asthma and chronic obstructive pulmonary disease. Advantageously, the presently claimed compounds may also exhibit one or more of the following properties: enhanced activity against GPR65 (also in native cells), better in vitro selectivity and toxicity profiles and/or enhanced oral pharmacokinetic profiles relevant to chronic once daily oral administration.

### Statement of Invention

A first aspect of the invention relates to a compound of formula (I), or a pharmaceutically acceptable salt or solvate thereof,

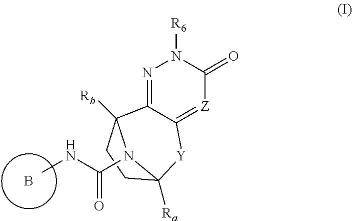

wherein:
ring B is:
   a monocyclic aromatic group; or
   a monocyclic or bicyclic heteroaromatic group,
each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
Z is CR₁₂;
Y is CR₁₀R_{10′}, wherein R₁₀ and R_{10′} are each independently selected from H, F, alkyl, and haloalkyl;
Rₐ and R_{b} are each independently selected from H and alkyl;
R₆ is selected from H, alkyl, cycloalkyl and hydroxyalkyl;
R₁₂ is selected from H, alkyl, haloalkyl, halo, OH and O-alkyl; and
R₁₃, R_{13′}, R₁₄, R_{14′}, R₁₅, R_{15′}, and R₁₆ are each independently selected from H, alkyl, and alkoxyalkyl.

Another aspect of the invention relates to a compound of formula (If), or a pharmaceutically acceptable salt or solvate thereof,

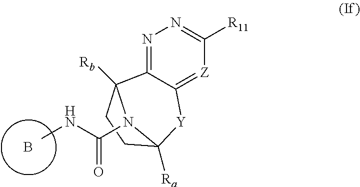

wherein:
R₁₁ is selected from H, alkyl, haloalkyl, halo, OH and O-alkyl; and
B, Y, Z, Rₐ and R_{b} are as defined hereinabove.

Another aspect of the invention relates to a compound of formula (Ij) or a pharmaceutically acceptable salt or solvate thereof,

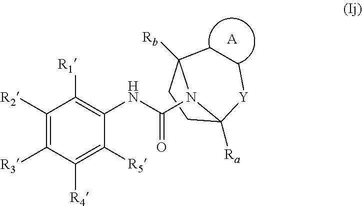

wherein ring A is selected from:

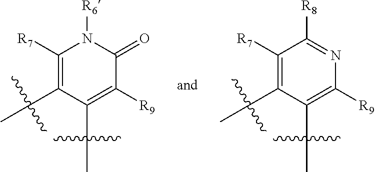

Y is CR₁₀R_{10′}, wherein R₁₀ and R_{10′} are each independently selected from H, F, alkyl, and haloalkyl;
Rₐ and R_{b} are each independently selected from H and alkyl;
R_{1′}, R_{2′}, R_{4′} and R_{5′} are each independently selected from H, CN, alkyl, alkoxy, haloalkyl, OH and halo;
R_{3′} is a pyridinyl group substituted by one or more substituents selected from CN, haloalkyl, haloalkoxy, NHCO-alkyl, NR₁₃R_{13′}, SO₂-alkyl, O—(CH₂)_{q}-heterocycloalkyl, alkoxy-alkoxy, alkylamino-alkoxy, dialkylamino-alkoxy, alkoxy-alkyl and O—(CH₂)ₚ-cycloalkyl, wherein said cycloalkyl group is optionally substituted by one or more halo, alkyl or alkoxy groups, and wherein said pyridinyl is optionally further substituted by one or more substituents selected from halo, alkyl and alkoxy;
R_{6′} is H or alkyl, more preferably H;
R₇, R₈ and R₉ are each independently selected from H, halo and alkyl;
R₁₃ and R_{13′} are each independently selected from H, alkyl, and alkoxy-alkyl; and
p and q are each independently 0 to 3.

Advantageously, the presently claimed compounds are capable of modulating GPR65, thereby rendering the compounds of therapeutic interest in the treatment of various disorders, for example, in the field of oncology, immuno-oncology, and immunology.

Another aspect of the invention relates to a compound or pharmaceutically acceptable salt or solvate thereof, as described herein for use as a medicament.

Another aspect of the invention relates to a compound or pharmaceutically acceptable salt or solvate thereof, as described herein for use in treating or preventing a disorder selected from a proliferative disorder, an immune disorder, asthma, chronic obstructive pulmonary disease (COPD) and acute respiratory distress syndrome (ARDS).

Another aspect of the invention relates to a pharmaceutical composition comprising a compound as described herein and a pharmaceutically acceptable diluent, excipient, or carrier.

Another aspect of the invention relates to a pharmaceutical composition as described herein for use as a medicament.

Another aspect of the invention relates to a pharmaceutical composition as described herein for use in treating or preventing a disorder selected from a proliferative disorder, an immune disorder, asthma, chronic obstructive pulmonary disease (COPD) and acute respiratory distress syndrome (ARDS).

Another aspect of the invention relates to a method of treating a disorder, comprising administering to a subject a compound or a pharmaceutical composition as described herein.

### DETAILED DESCRIPTION

The present invention relates to compounds that are capable of modulating GPR65.

“Alkyl” is defined herein as a straight-chain or branched alkyl radical, preferably C₁₋₂₀ alkyl, more preferably C₁₋₁₂ alkyl, even more preferably C₁₋₁₀ alkyl or C₁₋₆ alkyl, for example, methyl, ethyl, propyl, isopropyl, butyl, isobutyl, tert-butyl, pentyl, hexyl. More preferably, the alkyl is a C₁₋₃ alkyl.

“Cycloalkyl” is defined herein as a monocyclic alkyl ring, preferably, C₃₋₇-cycloalkyl, more preferably C₃₋₆-cycloalkyl. Preferred examples include cyclopropyl, cyclobutyl, cyclopentyl, cyclohexyl or cycloheptyl, or a fused bicyclic ring system such as norbornane.

As used herein, the term “aryl” refers to a C₆₋₁₂ aromatic group, which may be a monocyclic or fused bicyclic group, including benzocondensed groups. Examples include phenyl and naphthyl.

“Haloalkyl” is defined herein as a straight-chain or branched alkyl radical as defined above, for example, methyl, ethyl, propyl, isopropyl, butyl, isobutyl, tert-butyl, pentyl, hexyl, that is substituted with one or more halogen atoms (that may be the same or different), such as fluorine, chlorine, bromine, and iodine. Preferably, the haloalkyl is a C₁₋₂₀ haloalkyl, more preferably a C₁₋₁₂ haloalkyl, even more preferably a C₁₋₁₀ haloalkyl or a C₁₋₆ haloalkyl, or a C₁₋₃ haloalkyl. Preferred examples are CF₃ and CHF₂, with CF₃ being particularly preferred.

“Alkoxy” is defined herein as an oxygen atom bonded to an alkyl group as defined above, for example methoxy, ethoxy, propoxy, isopropoxy, butoxy, isobutoxy, tert-butoxy, pentoxy and hexoxy. Preferably, the alkoxy is a C₁₋₂₀ alkoxy, more preferably a C₁₋₁₂ alkoxy, even more preferably C₁₋₁₀ alkoxy or a C₁₋₆ alkoxy, or a C₁₋₃ alkoxy. A particularly preferred example is methoxy (—OCH₃). “Alkoxy-alkyl” is defined as an alkyl group as that is substituted by one or more alkoxy groups, e.g. MeOCH₂CH₂—. “Alkoxy-alkoxy” is defined as an alkoxy group that is substituted by one or more further alkoxy groups, e.g. MeOCH₂CH₂O— (also referred to as an ether group).

“Haloalkoxy” is defined herein as an alkoxy group as described above that is substituted with one or more halogen atoms (that may be the same or different), such as fluorine, chlorine, bromine, and iodine.

“Heteroaryl” or “heteroaromatic” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. Preferably, the heterocycloalkyl group is monocyclic or bicyclic. Preferably, the heterocycloalkyl group is a C₃₋₇-heterocycloalkyl, more preferably a C₃₋₆ heterocycloalkyl. Alternatively, the heterocycloalkyl group is a C₄₋₇-heterocycloalkyl, more preferably a C₄₋₆-heterocycloalkyl. Preferred heterocycloalkyl groups include, but are not limited to, piperazinyl, piperidinyl, morpholinyl, thiomorpholinyl, pyrrolidinyl, tetrahydrofuranyl and tetrahydropyranyl. Examples of heterocycloalkyl groups containing a CO group and one or more double bonds include 3-oxo-3,4-dihydro-2H-benzo[b][1,4]oxazin-6-yl, oxoisoindolinyl, oxoindolinyl, 1-oxo-1,2,3,4-tetrahydroiso-quinolin-6-yl, 1-oxo-1,2,3,4-tetrahydroisoquinolin-6-yl and the like.

“Aralkyl” is defined herein as an alkyl group as defined above substituted by one or more aryl groups as defined above.

Preferably alkyl is C₁-C₆ alkyl, haloalkyl is C₁-C₆ haloalkyl, alkoxy is C₁₋₆ alkoxy and haloalkoxy is C₁-C₆ haloalkoxy.

### Structural Representation of the Compounds

The compounds of the invention comprise a structure wherein an optionally substituted 6-membered nitrogen-containing ring is fused to a bicyclic nitrogen-containing moiety to form a tricyclic structure. The resulting tricyclic structure can exist in two different configurations as depicted below:

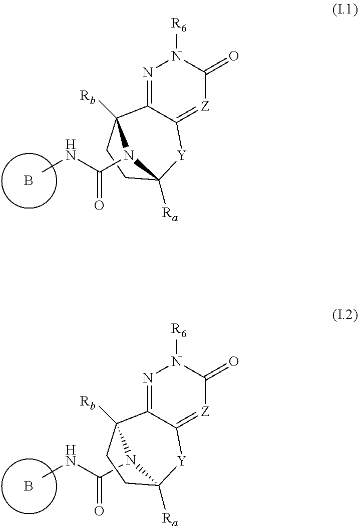

For the avoidance of doubt, the invention encompasses the compounds in either of the above configurations, as well as mixtures thereof, including racemic mixtures.

Alternatively, the structure can, for example, be represented, as follows (where Rₐ and R_{b} groups are omitted for clarity):

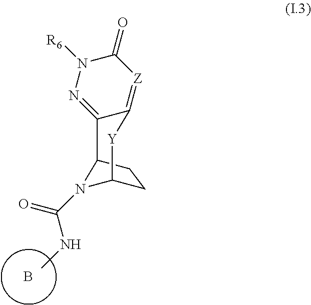

For the avoidance of doubt, the invention encompasses the compounds in the above configuration, as well the corresponding enantiomers thereof, and mixtures thereof, including racemic mixtures. As used throughout, and for ease of representation, specific examples of compounds according to the invention depicted in the above configuration (I.3) refer to mixtures of both enantiomers (in particular, the racemate), whereas the respective enantiomers—where these have been synthesised or separated—are depicted as either configuration (I.1) or configuration (I.2) with wedged bonds or dashed bonds respectively.

The compounds described herein contain an optionally substituted 6-membered ring, which is fused to the bicyclic nitrogen-containing moiety to form a tricyclic structure. The 6-membered ring contains a C═O group and can exist in more than one tautomeric form. For example, compounds of formula (I) where R₆ is H can exist in the following tautomeric forms:

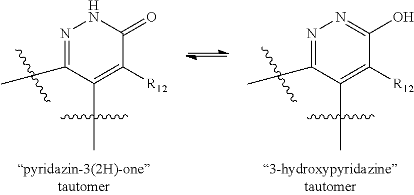

The pyridazin-3(2H)-one tautomer is believed to be the predominant solid state form. In solution, the energy difference between the two tautomeric forms is understood to be very small and is dependent on the polarity of the solvent. The invention encompasses all tautomeric forms of the compounds described herein.

### Compounds of Formula (I)

The present invention relates to compounds of formula (I) as defined above.

For the embodiments described throughout, preferably p and q are each independently 0 or 1. In one preferred embodiment, p and q are both 0.

In a preferred embodiment, the invention relates to a compound of formula (I), or a pharmaceutically acceptable salt or solvate thereof, wherein:
ring B is:
   a monocyclic aromatic group; or
   a monocyclic or bicyclic heteroaromatic group,
each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, O-heteroaryl, CONH-aryl, aryloxy-alkyl, O-aralkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, and heteroaryl;
m is an integer from 1 to 3;
Z is CR₁₂;
Y is CR₁₀R_{10′}, wherein R₁₀ and R_{10′} are each independently selected from H, F, alkyl, and haloalkyl;
Rₐ and R_{b} are each independently selected from H and alkyl;
R₆ is selected from H, alkyl, cycloalkyl and hydroxyalkyl;
R₁₂ is selected from H, alkyl, haloalkyl, halo, OH and O-alkyl; and
R₁₃, R_{13′}, R₁₄, R_{14′}, R₁₅, and R_{15′} and R₁₆ are each independently selected from H, alkyl, and alkoxyalkyl.

In a preferred embodiment, the invention relates to a compound of formula (I), or a pharmaceutically acceptable salt or solvate thereof, wherein:
ring B is:
   a monocyclic aromatic group; or
   a monocyclic or bicyclic heteroaromatic group,
each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl and CO₂-alkyl, wherein said aryl, heteroaryl, heterocycloalkyl, O-cycloalkyl and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, and heteroaryl;
m is an integer from 1 to 3;
Z is CR₁₂;
Y is CR₁₀R_{10′}, wherein R₁₀ and R_{10′}, are each independently selected from H, F, alkyl, and haloalkyl;
Rₐ and R_{b} are each independently selected from H and alkyl;
R₆ is selected from H, alkyl, cycloalkyl and hydroxyalkyl;
R₁₂ is selected from H, alkyl, haloalkyl, halo, OH and O-alkyl; and
R₁₃, R_{13′}, R₁₄, R_{14′}, R₁₅, and R_{15′} and R₁₆ are each independently selected from H, alkyl, and alkoxyalkyl.

In one preferred embodiment, m is an integer from 1 to 3, more preferably 1 or 2, more preferably 1.

Throughout each of the embodiments described herein, preferably R₁₃, R_{13′}, R₁₄, R_{14′}, R₁₅, R_{15′} and R₁₆ are each independently selected from H and alkyl. More preferably, R₁₃, R_{13′}, R₁₄, R_{14′}, R₁₅, R_{15′} are all H. In one preferred embodiment, R₁₆ is alkyl, more preferably Me.

In one preferred embodiment,
ring B is:
   a monocyclic aromatic group; or
   a monocyclic or bicyclic heteroaromatic group,
each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl and CO₂-alkyl, wherein said aryl, heteroaryl, heterocycloalkyl, O-cycloalkyl and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl;
Z is CR₁₂;
Y is CR₁₀R_{10′}, wherein R₁₀ and R_{10′} are each independently selected from H, F, alkyl, and haloalkyl;
Rₐ and R_{b} are each independently selected from H and alkyl;
R₆ is selected from H, alkyl, cycloalkyl and hydroxyalkyl;
R₁₂ is selected from H, alkyl, haloalkyl, halo, OH and O-alkyl; and
R₁₃, R_{13′}, R₁₄, R_{14′}, R₁₅, and R_{15′} are each independently selected from H, alkyl, and alkoxyalkyl.

In one preferred embodiment,
ring B is:
   a monocyclic aromatic group; or
   a monocyclic or bicyclic heteroaromatic group,
each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl and CO₂-alkyl, wherein said aryl, heteroaryl, O-cycloalkyl and O-aryl groups are each optionally further substituted by one or more groups independently selected from halo, alkyl and alkoxy;
Z is CR₁₂;
Y is CR₁₀R_{10′}, wherein R₁₀ and R_{10′} are each independently selected from H, F, alkyl, and haloalkyl;
Rₐ and R_{b} are each independently selected from H and alkyl;
R₆ is selected from H, alkyl, cycloalkyl and hydroxyalkyl; and
R₁₂ is selected from H, alkyl, haloalkyl, halo, OH and O-alkyl.

In one preferred embodiment, Rₐ and R_{b} are each independently selected from H and methyl. In one preferred embodiment, one of Rₐ and R_{b} is alkyl (more preferably methyl) and the other is H. In one particularly preferred embodiment, Rₐ and R_{b} are both H.

In one preferred embodiment, Y is CR₁₀R_{10′}, where R₁₀ and R_{10′} are each independently selected from H, F, Me and CF₃, and more preferably selected from H, F and Me. In one preferred embodiment, Y is selected from CH₂, CHF and CHMe. More preferably, Y is CH₂.

In one preferred embodiment, R₆ is selected from H, methyl and hydroxymethyl, and is more preferably H.

In one preferred embodiment, R₁₂ is H or alkyl.

In one particularly preferred embodiment, Z is CH (i.e. R₁₂ is H), and the compound is of formula (Ie), or a pharmaceutically acceptable salt or solvate thereof:

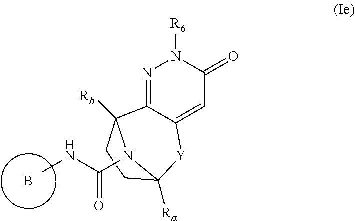

wherein R₆, Rₐ, R_{b}, Y and B are as defined above.

In one preferred embodiment, ring B is a phenyl group optionally substituted by one or more substituents each independently selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl and CO₂-alkyl, wherein said aryl, heteroaryl, O-cycloalkyl and O-aryl groups are each optionally further substituted by one or more groups independently selected from halo, alkyl and alkoxy.

In one preferred embodiment, the compound is of formula (Ia), or a pharmaceutically acceptable salt or solvate thereof,

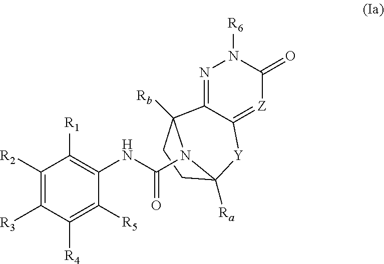

wherein
R₁, R₄, and R₅ are each independently selected from H, CN, alkyl, alkoxy, haloalkyl, OH, and halo;
R₂ and R₃ are each independently selected from H, OH, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, haloalkyl, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

Preferably, for compounds of formula (Ia):
R₁, R₄, and R₅ are each independently selected from H, CN, alkyl, alkoxy, haloalkyl, OH, and halo;
R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, O-heteroaryl, CONH-aryl, aryloxy-alkyl, O-aralkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, and heteroaryl;
Z, Y, Rₐ, R_{b}, m and R₆ are as defined above; and
R₁₃, R_{13′}, R₁₄, R_{14′}, R₁₅, R_{15′} and R₁₆ are each independently selected from H, alkyl, and alkoxyalkyl.

Preferred definitions for R₆, Rₐ, R_{b}, Y and Z as defined above apply equally to compounds of formula (Ia).

In one preferred embodiment, R₃ is an aryl or heteroaryl group each of which is optionally further substituted by one or more groups independently selected from halo, haloalkyl, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, 0-(CH₂)ₚ-cycloalkyl, where in the latter group, said cycloalkyl group is optionally further substituted by one or more halo, haloalkyl, alkyl or alkoxy groups.

In one preferred embodiment, R₃ is selected from phenyl, pyridyl, pyrimidinyl, pyrazolyl, pyrazinyl, [1,2,5]thiadiazolo[3,4-b]pyridinyl, indazolyl, triazolyl, benzotriazolyl, oxoisoindolinyl, oxoindolinyl, imidazolyl, benzoxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, benzo[c]isothiazolyl, imidazo[1,5-a]pyridinyl, O-pyridinyl, CONHPh, NHCOPh, OCH₂Ph, CH₂OPh, [1,2,5]oxadiazolo[3,4-b]pyridinyl, benzo[c]isoxazolyl, or 2H-benzo[b][1,4]oxazin-3(4H)-onyl; each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, and heteroaryl.

In one preferred embodiment, R₃ is selected from phenyl, pyridyl, pyrimidinyl, pyrazolyl, indazolyl, triazolyl, benzotriazolyl, oxoisoindolinyl, oxoindolinyl, imidazolyl, benzoxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, imidazo[1,5-a]pyridinyl, [1,2,5]oxadiazolo[3,4-b]pyridinyl, benzo[c]isoxazolyl, or 2H-benzo[b][1,4]oxazin-3(4H)-onyl; each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, alkyl-NHSO₂-alkyl, CO₂R₁₆, alkyl-alkoxy, O-cycloalkyl, haloalkoxy, and heteroaryl.

In one preferred embodiment, R₃ is selected from phenyl, pyridyl, pyrimidinyl, pyrazolyl, indazolyl, triazolyl, benzotriazolyl, oxoisoindolinyl, oxoindolinyl, imidazolyl, benzoxazinyl, pyrrolopyridinyl, oxotetrohydroisoquinolinyl, benzo[c][1,2,5]oxadiazolyl, or 2H-benzo[b][1,4]oxazin-3(4H)-onyl; each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl.

In compounds of formula (Ia), preferably R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl, heterocycloalkyl, O-cycloalkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl and heteroaryl. Preferably, said aryl, heteroaryl, heterocycloalkyl, O-cycloalkyl, and O-aryl groups are each optionally further substituted by one or more groups independently selected from halo, alkyl and alkoxy.

In one preferred embodiment, R₁ and R₄ are both H.

In one preferred embodiment, R₅ is selected from H, F, Me, MeO, Cl, OH and CN, and is preferably H or F, more preferably F.

In one preferred embodiment, R₂ is selected from Cl, Br, and CF₃, more preferably Cl.

In another preferred embodiment, R₁ and R₄ are selected from halo and haloalkyl (more preferably halo) and R₂ and R₅ are both H.

In one preferred embodiment, R₁ is F, R₂ is H, R₄ is Cl and R₅ is H, i.e. ring B is:

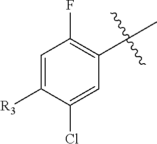

Preferably R₃ is defined as set out below.

In one preferred embodiment of formula (Ia), R₃ is selected from:

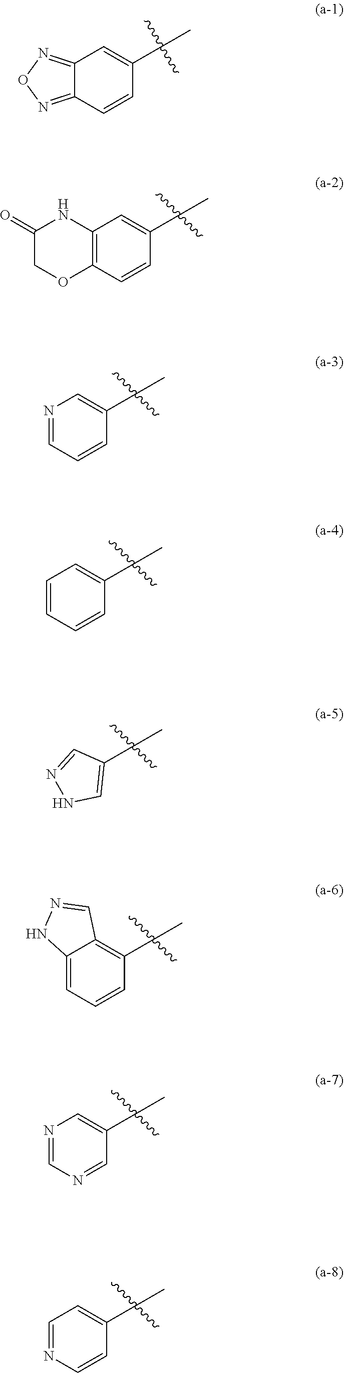

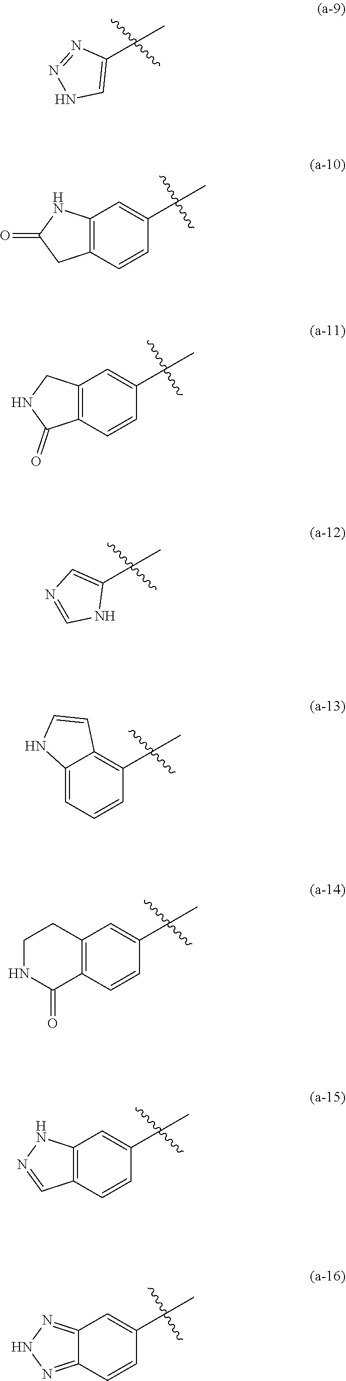

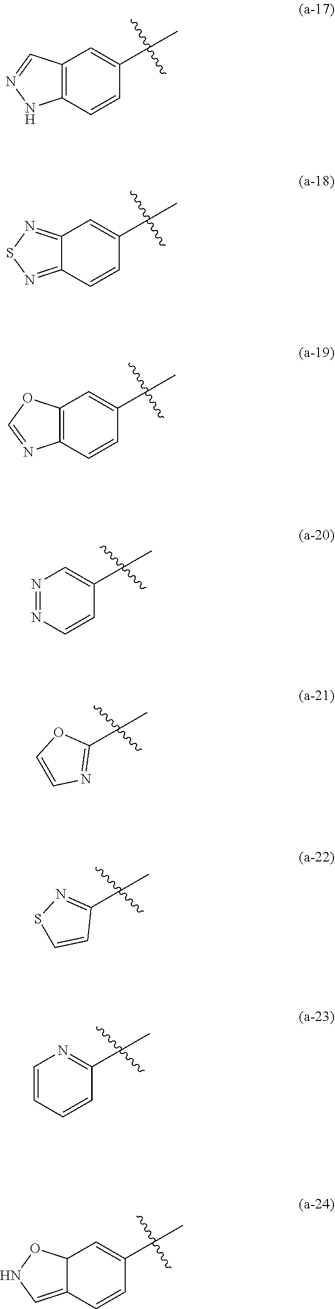

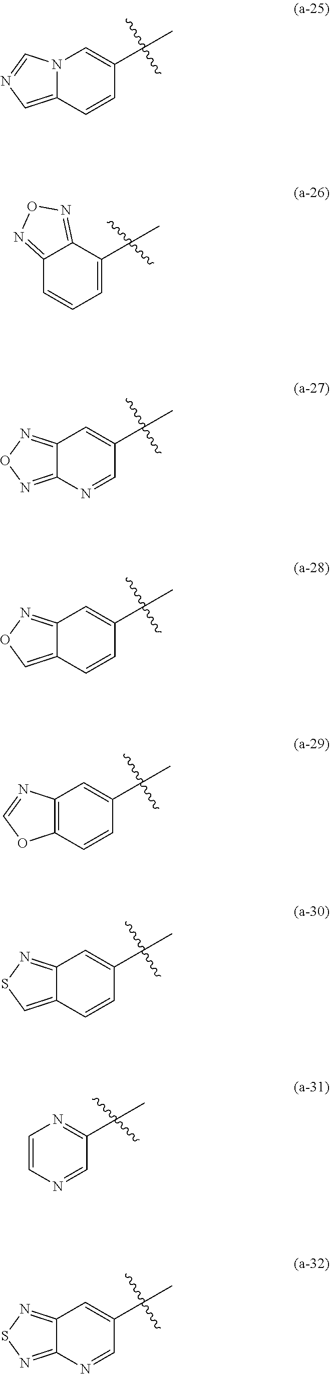

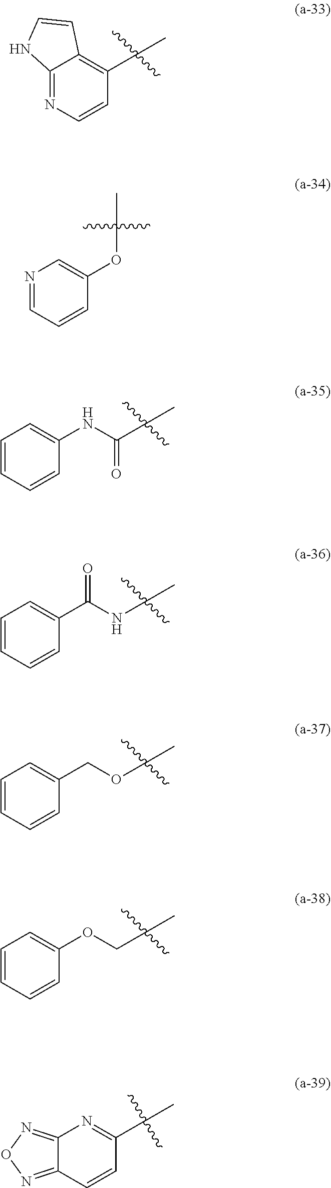

each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, O—(CH₂)ₚ-cycloalkyl, where in the latter group, said cycloalkyl group is optionally further substituted by one or more halo, haloalkyl, alkyl or alkoxy groups. Preferably, R₁ and R₄ are selected from halo and haloalkyl (more preferably halo) and R₂ and R₅ are both H. More preferably, R₁ is F, R₂ is H, R₄ is Cl and R₅ is H.

In one preferred embodiment, R₃ is selected from the above groups (a-1)-(a-38), each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, heterocycloalkyl, and heteroaryl, wherein R₁₃, R_{13′}, R₁₄, R_{14′}, R₁₅, R_{15′} and R₁₆ are each independently selected from H, alkyl, and alkoxyalkyl. Preferably, R₁ and R₄ are selected from halo and haloalkyl (more preferably halo) and R₂ and R₅ are both H. More preferably, R₁ is F, R₂ is H, R₄ is Cl and R₅ is H.

In one preferred embodiment, R₃ is selected from the above groups (a-1)-(a-28), each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy and heteroaryl, wherein R₁₃, R_{13′}, R₁₄, R_{14′}, R₁₅, R_{15′} and R₁₆ are each independently selected from H, alkyl, and alkoxyalkyl. Preferably, R₁ and R₄ are selected from halo and haloalkyl (more preferably halo) and R₂ and R₅ are both H. More preferably, R₁ is F, R₂ is H, R₄ is Cl and R₅ is H.

In one preferred embodiment, R₃ is selected from the above groups, each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl and heteroaryl. In one preferred embodiment, R₃ is selected from groups (a-1)-(a-16) above, each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl and heteroaryl. In one preferred embodiment, R₃ is a pyridinyl group, optionally substituted as above for definitions (a-1)-(a-39). More preferably, R₃ is a 3-pyridinyl group, optionally substituted as above for definitions (a-1)-(a-39). Preferably, R₁ and R₄ are selected from halo and haloalkyl (more preferably halo) and R₂ and R₅ are both H. More preferably, R₁ is F, R₂ is H, R₄ is Cl and R₅ is H.

In one preferred embodiment, in formula (Ia), R₁ is F; R₂ is H; R₃ is an optionally substituted pyridinyl group; R₄ is Cl or CF₃; and R₅ is H. More preferably, R₁ is F; R₂ is H; R₃ is an optionally substituted pyridinyl group; R₄ is Cl; and R₅ is H.

In one preferred embodiment, in formula (Ia), R₃ is:

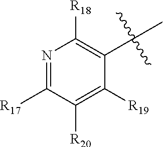

wherein:
R₁₇ is selected from H, alkyl, CN, haloalkyl, NHCO-alkyl, NR₁₃R_{13′}, alkoxy, SO₂-alkyl, halo, O—(CH₂)_{q}-heterocycloalkyl, alkoxy-alkoxy, alkylamino-alkoxy, dialkylamino-alkoxy, alkoxy-alkyl, haloalkoxy and O—(CH₂)ₚ-cycloalkyl, wherein the cycloalkyl group is optionally substituted by one or more halo, alkyl or alkoxy groups;
R₁₈ is selected from H and halo;
R₁₉ is selected from H, alkoxy and alkoxy-alkyl; and
R₂₀ is selected from H, halo and CO₂R₁₆. Preferably, R₁ and R₄ are selected from halo and haloalkyl (more preferably halo) and R₂ and R₅ are both H. More preferably, R₁ is F, R₂ is H, R₄ is Cl and R₅ is H.

In one preferred embodiment, R₁₇ is selected from H, alkyl, CN, haloalkyl, NHCO-alkyl, NR₁₃R_{13′}, alkoxy, SO₂-alkyl, halo, O—(CH₂)_{q}-heterocycloalkyl, alkoxy-alkoxy, alkoxy-alkyl, haloalkoxy and O—(CH₂)ₚ-cycloalkyl, wherein the cycloalkyl group is optionally substituted by one or more halo, alkyl or alkoxy groups

In one preferred embodiment, R₁₇ is selected from H, alkyl, CN, haloalkyl, NHCO-alkyl, NR₁₃R_{13′}, alkoxy, SO₂-alkyl, halo, O-heterocycloalkyl, alkoxy-alkoxy, alkoxy-alkyl, haloalkoxy and O-cycloalkyl, wherein the cycloalkyl group is optionally substituted by one or more halo, alkyl or alkoxy groups.

In one preferred embodiment, R₁₇ is selected from haloalkyl and haloalkoxy, more preferably fluoroalkyl and fluoroalkoxy.

In one preferred embodiment, R₁₇ is selected from —O—CH₂CF₃, —O—CH₂CH₂CF₃ and —OCHF₂, —OCH₂CHF₂.

In one preferred embodiment, R₁₇ is selected from fluoroalkyl, fluoroalkoxy, methylamino-alkoxy, dimethylamino-alkoxy, (piperidin-1-yl)-alkoxy, O—(CH₂)ₚ-cyclopropyl, O—(CH₂)ₚ-cyclobutyl, wherein said cyclopropyl and cyclobutyl group is optionally substituted by an alkoxy group; and R₁₈, R₁₉ and R₂₀ are all H.

In one preferred embodiment, R₁₇ is selected from CF₃, —O—CH₂CF₃, —O—CH₂CH₂CF₃, —OCHF₂, —OCH₂CHF₂, —OCH₂CH₂NHMe, —OCH₂CH₂NHMe, (piperidin-1-yl)-CH₂CH₂—O—, —O— cyclopropyl, —O—CH₂-cyclopropyl, methoxycyclobutyl-O—; and R₁₈, R₁₉ and R₂₀ are all H.

In one preferred embodiment, R₁₇ is selected from H, alkoxy, SO₂-alkyl, halo, O-cycloalkyl, O-heterocycloalkyl and haloalkoxy,
R₁₈ is selected from H and halo;
R₁₉ is selected from H, alkoxy and alkoxy-alkyl; and
R₂₀ is selected from H, halo and CO₂R₁₆.

In one preferred embodiment, R₁₇ is selected from H, OMe, OEt, OⁱPr, F, SO₂Me, halo, O-cyclobutyl, O-oxetanyl, and O—CH₂CF₃;
R₁₈ is selected from H and F;
R₁₉ is selected from H and MeOCH₂—;
R₂₀ is selected from H, F and CO₂Me;
R₁ is F;
R₂ is H;
R₄ is Cl or CF₃; and
R₅ is H.

In one preferred embodiment, R₁₇ is selected from H, OMe, OEt, OⁱPr, F, SO₂Me, halo, O-cyclobutyl, O-oxetanyl, and O—CH₂CF₃;
R₁₈ is selected from H and F;
R₁₉ is selected from H and MeOCH₂—;
R₂₀ is selected from H, F and CO₂Me;
R₁ is F;
R₂ is H;
R₄ is Cl or CF₃; and
R₅ is H.

In one preferred embodiment, the compound is of formula (Ia), or a pharmaceutically acceptable salt or solvate thereof, wherein:
R₁, R₄, and R₅ are each independently selected from H, CN, alkyl, alkoxy, haloalkyl, OH, and halo;
R₂ and R₃ are each independently selected from H, halo, CN, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl and O-aryl groups are each optionally further substituted by one or more groups independently selected from halo, alkyl and alkoxy; and
Z, Y, Rₐ, R_{b}, and R₆ are as defined above.

Preferably, for compounds of formula (Ia), Z is CH.

In one preferred embodiment, at least one of R₁, R₂, R₃, R₄ and R₅ is other than H.

In one preferred embodiment, one of R₁, R₂, R₃, R₄ and R₅ is other than H.

In one preferred embodiment, two of R₁, R₂, R₃, R₄ and R₅ are other than H.

In one preferred embodiment, three of R₁, R₂, R₃, R₄ and R₅ are other than H.

In one preferred embodiment, R₂ and R₃ are each independently selected from H, F, Cl, Br, CN, methoxy, OCF₃, CF₃, OCHF₂, Me, Ph, pyrazolyl, oxazolyl, thiazolyl, OPh, NHCO—CH═CH₂ and CO₂Me, wherein said Ph, OPh, pyrazolyl, oxazolyl and thiazolyl groups are each optionally further substituted by one or more alkyl groups.

In one preferred embodiment, R₂ and R₃ are each independently selected from H, F, Cl, Br, I, CN, methoxy, haloalkyl, haloalkoxy and CO₂-alkyl.

In one preferred embodiment, R₃ is selected from H, F, Cl, Br, CN, methoxy, OCF₃, CF₃, OCHF₂, Me, Ph, pyrazolyl, oxazolyl, thiazolyl, OPh, NHCO—CH═CH₂ and CO₂Me, wherein said Ph, OPh, pyrazolyl, oxazolyl and thiazolyl groups are each optionally further substituted by one or more alkyl groups. Preferably the pyrazolyl is a 1H-pyrazol-1-yl group. Preferably, the oxazolyl group is an oxazol-5-yl group. Preferably, the thiazolyl group is a thiazol-4-yl group.

In one preferred embodiment, R₂ is selected from H, F, C, Br, CN, Me, methoxy, OCF₃, CF₃, OCHF₂, Ph, pyrazolyl and CO₂Me, wherein said Ph and pyrazolyl groups are each optionally further substituted by one or more alkyl groups. Preferably the pyrazolyl is a 1H-pyrazol-1-yl group.

In one preferred embodiment, R₂ and R₃ are each independently selected from F, C, Br, I, CN, CO₂-alkyl, C₁-C₆ haloalkyl and C₁-C₆ haloalkoxy.

In one preferred embodiment, R₂ and R₃ are each independently selected from F, C, Br, I, CN, C₁-C₆ haloalkyl, C₁-C₆ haloalkoxy and CO₂-alkyl, more preferably, Cl, Br, and CF₃, even more preferably Cl and CF₃.

In one preferred embodiment, R₂ and R₃ are each independently selected from F, Cl, Br, I, CN, and C₁-C₆ haloalkyl.

In one preferred embodiment, R₂ and R₃ are each independently selected from H, F, Cl, Br, I, CN, methoxy, and haloalkyl.

In one preferred embodiment, R₂ and R₃ are each independently selected from CN, Cl, Br, OCF₃ and CF₃, and more preferably are each independently selected from Cl and CF₃.

In one preferred embodiment, R₂ and R₃ are each independently selected from Cl, Br, and CF₃, and more preferably independently selected from Cl and CF₃.

In one preferred embodiment, one of R₂ and R₃ is Cl and the other is OCF₃ or OCHF₂.

In one preferred embodiment, one of R₂ and R₃ is Cl and the other is CO₂Me.

In one preferred embodiment, R₂ and R₃ are both Cl, or one of R₂ and R₃ is Cl and the other is CF₃.

In one preferred embodiment, R₂ and R₃ are both Cl.

In one preferred embodiment, one of R₂ and R₃ is Cl, and the other is CF₃.

In one preferred embodiment, one of R₂ and R₃ is CN, and the other is CF₃.

In one preferred embodiment, one of R₂ and R₃ is Br, and the other is CF₃.

In one preferred embodiment, one of R₂ and R₃ is Cl, and the other is OCF₃.

In one preferred embodiment, one of R₂ and R₃ is Cl, and the other is Br.

In one preferred embodiment, one of R₂ and R₃ is Cl and the other is selected from OCF₃, CO₂Me, OCHF₂ and CF₃.

In one preferred embodiment, R₁, R₄, and R₅ are each independently selected from H, alkyl, alkoxy, OH, F, C, Br, and I.

In one preferred embodiment, R₁, R₄, and R₅ are each independently selected from H, Me, OMe, OH, F, C, Br, and I.

In one preferred embodiment, R₁, R₄, and R₅ are each independently selected from H, F, C, Br, and I.

In one preferred embodiment R₅ is selected from H, F, Me, MeO and C, and is preferably H or F, more preferably H.

In one preferred embodiment R₅ is selected from H, F and Cl, and is preferably H or F, more preferably H.

In one preferred embodiment, R₅ is selected from H, F and CN, and is preferably H.

In one preferred embodiment, R₁ and R₄ are both H.

In one preferred embodiment, R₁ is H; R₂ is selected from H, F, Cl, Br, CN, Me, methoxy, OCF₃, CF₃, OCHF₂, Ph, pyrazolyl and CO₂Me; R₃ is selected from H, F, Cl, Br, CN, methoxy, OCF₃, CF₃, OCHF₂, Me, Ph, pyrazolyl, oxazolyl, thiazolyl, OPh, NHCO—CH═CH₂ and CO₂Me, wherein said Ph, OPh, pyrazolyl, oxazolyl and thiazolyl groups are each optionally further substituted by one or more alkyl groups; R₄ is H or CF₃, more preferably H; R₅ is selected from H, F, Me, MeO, Cl, OH and CN, and is preferably H or F, more preferably H. Preferably, for this embodiment, at least one of R₂ and R₃ is other than H. Even more preferably, both R₂ and R₃ are other than H.

In another preferred embodiment, one of R₂ and R₃ is selected from aryl, O-aryl and heteroaryl, each of which is optionally substituted, and the other of R₂ and R₃ is H, and R₁, R₄ and R₅ are all H.

In one preferred embodiment, the compound is of formula (Ib), or a pharmaceutically acceptable salt or solvate thereof,

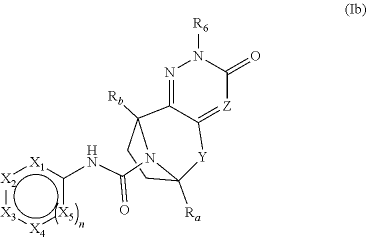

wherein where n is 0 or 1 and X₁-X₅ form a 5- or 6-membered heteroaromatic group containing at least one nitrogen atom, said heteroaromatic group being optionally substituted by one or more substituents selected from halo, CN, OH, alkyl, alkoxy, haloalkyl, haloalkoxy, aryl, heteroaryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and O-aryl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, CO₂-alkyl, and O-aryl groups are each optionally further substituted by one or more groups independently selected from halo, haloalkyl, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

In one preferred embodiment, the compound is of formula (Ib), or a pharmaceutically acceptable salt or solvate thereof, wherein where n is 0 or 1 and X₁-X₅ form a 5- or 6-membered heteroaromatic group containing at least one nitrogen atom, said heteroaromatic group being optionally substituted by one or more substituents selected from halo, CN, alkyl, alkoxy, haloalkyl, aryl, heteroaryl, heterocycloalkyl, O-heterocycloalkyl, cycloalkyl, halocycloalkyl, hydroxycycloalkyl, O-cycloalkyl, NHCO-aryl, O-heteroaryl, CONH-aryl, aryloxy-alkyl, O-aralkyl, and O-aryl, wherein said aryl, heteroaryl, heterocycloalkyl, O-cycloalkyl, NHCO-aryl, O-heteroaryl, CONH-aryl, aryloxy-alkyl, O-aralkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, and heteroaryl; Z, Y, Rₐ, R_{b}, and R₆ are as defined above; and R₁₃, R_{13′}, R₁₄, R_{14′}, R₁₅, and R_{15′} are each independently selected from H, alkyl, and alkoxyalkyl.

Preferred definitions for R₆, Rₐ, R_{b}, Y and Z as defined above apply equally to compounds of formula (Ib).

In one preferred embodiment, n is 0 or 1 and X₁-X₅ form a 5- or 6-membered heteroaromatic group containing at least one nitrogen atom, said heteroaromatic group being optionally substituted by one or more substituents selected from halo, CN, alkyl, alkoxy, haloalkyl, aryl, heteroaryl, heterocycloalkyl, O-heterocycloalkyl, cycloalkyl, halocycloalkyl, hydroxycycloalkyl, O-cycloalkyl and O-aryl, wherein said aryl, heteroaryl, heterocycloalkyl, O-cycloalkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy and heteroaryl.

In one preferred embodiment, n is 0 or 1 and X₁-X₅ form a 5- or 6-membered heteroaromatic group containing at least one nitrogen atom, said heteroaromatic group being optionally substituted by one or more substituents selected from halo, CN, alkyl, alkoxy, haloalkyl, aryl, heteroaryl, heterocycloalkyl, O-heterocycloalkyl, cycloalkyl, halocycloalkyl, hydroxycycloalkyl, O-cycloalkyl and O-aryl, wherein said aryl, heteroaryl, heterocycloalkyl, O-cycloalkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl and heteroaryl.

In one preferred embodiment of formula (Ib), X₃ is CR₃, wherein R₃ is selected from phenyl, pyridyl, pyrimidinyl, pyrazolyl, indazolyl, triazolyl, benzotriazolyl, oxoisoindolinyl, oxoindolinyl, imidazolyl, benzoxazinyl, pyrrolopyridinyl, oxotetrohydroisoquinolinyl, benzo[c][1,2,5]oxadiazolyl, or 2H-benzo[b][1,4]oxazin-3(4H)-onyl; each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl.

In one preferred embodiment of formula (Ib), X₁ is CR₁ and X₄ is CR₄, wherein R₁ and R₄ are both H.

In one preferred embodiment of formula (Ib), X₅ is CR₅, wherein R₅ is selected from H, F, Me, MeO, Cl, OH and CN, and is preferably H or F, more preferably F.

In one preferred embodiment of formula (Ib), X₂ is CR₂, wherein R₂ is selected from Cl, Br, and CF₃, more preferably C₁.

In one preferred embodiment of formula (Ib), X₃ is CR₃, and R₃ is as defined as for formula (Ia) above.

In one preferred embodiment of formula (Ib), X₃ is CR₃, wherein R₃ is selected from (a-1)-(a-38) as described above, more preferably (a-1)-(a-28), even more preferably (a-1)-(a-16).

Preferably, R₃ is selected from (a-1)-(a-16), each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl, wherein R₁₃, R_{13′}, R₁₄, R_{14′}, R₁₅, and R_{15′} are each independently selected from H, alkyl, and alkoxyalkyl.

In another particularly preferred embodiment, the compound is of formula (Ib), or a pharmaceutically acceptable salt or solvate thereof, wherein where n is 0 or 1 and X₁-X₅ form a 5- or 6-membered heteroaromatic group containing at least one nitrogen atom, said heteroaromatic group being optionally substituted by one or more substituents selected from halo, CN, alkyl, alkoxy, haloalkyl, aryl, heteroaryl, heterocycloalkyl, O-heterocycloalkyl, cycloalkyl, halocycloalkyl, hydroxycycloalkyl, O-cycloalkyl and O-aryl; and Z, Y, Rₐ, R_{b}, and R₆ are as defined above.

Preferably, for compounds of formula (Ib), Z is CH.

Preferably, for compounds of formula (Ib), R₁ and R₄ are H.

In one preferred embodiment, n is 1, and X₁-X₅ form a 6-membered heteroaromatic group selected from pyridin-2-yl, pyridin-3-yl, pyridin-4-yl, pyrazin-2-yl, pyrazin-3-yl, pyrimidin-2-yl, pyrimidin-4-yl, pyrimidin-5-yl, 1,2,3-triazinyl, 1,2,4-triazinyl, 1,3,5-triazinyl, 1,2,4,5-tetrazinyl and 1,2,3,4-tetrazinyl, each of which is optionally substituted by one or more substituents selected from halo, CN, alkoxy, alkyl, haloalkyl, aryl, heteroaryl, heterocycloalkyl, O-heterocycloalkyl, cycloalkyl, halocycloalkyl, hydroxycycloalkyl, O-cycloalkyl, NHCO-aryl, O-heteroaryl, CONH-aryl, aryloxy-alkyl, O-aralkyl and O-aryl, more preferably, H, halo, CN, alkoxyl, alkyl and haloalkyl, wherein said aryl, heteroaryl, heterocycloalkyl, O-cycloalkyl, NHCO-aryl, O-heteroaryl, CONH-aryl, aryloxy-alkyl, O-aralkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, and heteroaryl.

In one preferred embodiment, n is 1, and X₁-X₅ form a 6-membered heteroaromatic group selected from pyridin-2-yl, pyridin-3-yl, pyridin-4-yl, pyrazin-2-yl, pyrazin-3-yl, pyrimidin-2-yl, pyrimidin-4-yl, pyrimidin-5-yl, 1,2,3-triazinyl, 1,2,4-triazinyl, 1,3,5-triazinyl, 1,2,4,5-tetrazinyl and 1,2,3,4-tetrazinyl, each of which is optionally substituted by one or more substituents selected from halo, CN, alkoxy, alkyl, haloalkyl, aryl, heteroaryl, heterocycloalkyl, O-heterocycloalkyl, cycloalkyl, halocycloalkyl, hydroxycycloalkyl, O-cycloalkyl and O-aryl, more preferably, H, halo, CN, alkoxyl, alkyl and haloalkyl.

In one preferred embodiment, the compound is of formula (Ic), or a pharmaceutically acceptable salt or solvate thereof,

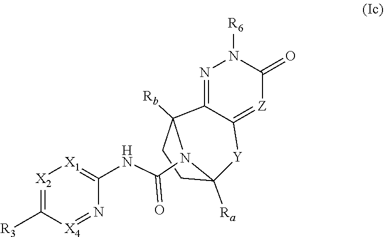

wherein:
X₁ is N or CR₁;
X₂ is N or CR₂;
X₄ is N or CR₄; and
R₁, R₂, R₃ and R₄ are each independently selected from H, halo, haloalkyl, alkyl, alkoxy, CN, aryl, heterocycloalkyl, heteroaryl and O-aryl, more preferably, H, halo, CN, alkoxyl, alkyl and haloalkyl, wherein said aryl, heteroaryl, heterocycloalkyl, O-cycloalkyl and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy and heteroaryl;
Z, Y, Rₐ, R_{b}, and R₆ are as defined above; and
R₁₃, R_{13′}, R₁₄, R_{14′}, R₁₅, and R_{15′} are each independently selected from H, alkyl, and alkoxyalkyl.

Preferred definitions for R₆, Rₐ, R_{b}, Y and Z as defined above apply equally to compounds of formula (Ic).

In one preferred embodiment of formula (Ic), R₁, R₂, R₃ and R₄ are each independently selected from H, halo, haloalkyl, alkyl, alkoxy, CN, aryl, heterocycloalkyl, heteroaryl and O-aryl, more preferably, H, halo, CN, alkoxyl, alkyl and haloalkyl, wherein said aryl, heteroaryl, heterocycloalkyl, O-cycloalkyl and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl.

In one preferred embodiment of formula (Ic), R₃ is selected from phenyl, pyridyl, pyrimidinyl, pyrazolyl, indazolyl, triazolyl, benzotriazolyl, oxoisoindolinyl, oxoindolinyl, imidazolyl, benzoxazinyl, pyrrolopyridinyl, oxotetrohydroisoquinolinyl, benzo[c][1,2,5]oxadiazolyl, or 2H-benzo[b][1,4]oxazin-3(4H)-onyl; each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl.

In one preferred embodiment of formula (Ic), X₁ is CR₁ and X₄ is CR₄, wherein R₁ and R₄ are both H.

In one preferred embodiment of formula (Ic), X₂ is CR₂, wherein R₂ is selected from Cl, Br, and CF₃, more preferably C₁.

In one preferred embodiment of formula (Ic), R₃ is as defined as for formula (Ia) above.

In one preferred embodiment of formula (Ic), X₃ is CR₃, wherein R₃ is selected from (a-1)-(a-38) as described above, more preferably (a-1) to (a-28), more preferably (a-1)-(a-16). In one preferred embodiment of formula (Ic), R₃ is selected from (a-1) to (a-28) each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, and heteroaryl, wherein R₁₃, R_{13′}, R₁₄, R_{14′}, R₁₅, and R_{15′} are each independently selected from H, alkyl, and alkoxyalkyl.

In one particularly preferred embodiment, the compound is of formula (Ic), or a pharmaceutically acceptable salt or solvate thereof, wherein:
X₁ is N or CR₁;
X₂ is N or CR₂;
X₄ is N or CR₄; and
R₁, R₂, R₃ and R₄ are each independently selected from H, halo, haloalkyl, alkyl, alkoxy, CN, aryl, heteroaryl and O-aryl, more preferably, H, halo, CN, alkoxyl, alkyl and haloalkyl; and
Z, Y, Rₐ, R_{b}, and R₆ are as defined above.

Preferably, for compounds of formula (Ic), Z is CH.

Preferably, for compounds of formula (Ic), R₁ and R₄ are H.

In one preferred embodiment, for compounds of formula (Ic), R₃ is selected from halo and haloalkyl, and is more preferably selected from Cl, F and CF₃.

In one preferred embodiment, the compound is of formula (Ic), wherein X₁ is N or CR₁, X₂ is N or CR₂ and X₄ is CR₄.

In one preferred embodiment, the compound is of formula (Ic), wherein X₁ is CR₁, X₂ is CR₂, and X₄ is CR₄. Preferably, for this embodiment, R₃ is selected from halo, haloalkyl, alkyl, alkoxy and CN. Even more preferably, R₃ is selected from halo and haloalkyl. More preferably still, R₃ is selected from Cl, F and CF₃, even more preferably, Cl and CF₃. Preferably, for this embodiment, R₁, R₂ and R₄ are each independently selected from H and halo, more preferably, H and Cl. More preferably, at least one of R₂ and R₄ is halo.

In one particularly preferred embodiment:
X₃ is CR₃, where R₃ is selected from halo, haloalkyl, alkyl, alkoxy and CN;
X₁ is CR₁, where R₁ is H;
X₂ is CR₂, where R₂ is selected from H, halo, haloalkyl, alkyl, alkoxy and CN;
X₄ is CR₄, where R₄ is selected from H, halo, haloalkyl, alkyl, alkoxy and CN;
wherein at least one of R₂ and R₄ is other than H.

In one particularly preferred embodiment:
X₃ is CR₃, where R₃ is selected from halo and haloalkyl;
X₁ is CR₁, where R₁ is H;
X₂ is CR₂, where R₂ is selected from H, halo and haloalkyl;
X₄ is CR₄, where R₄ is selected from H, halo and haloalkyl;
wherein at least one of R₂ and R₄ is other than H.

In one particularly preferred embodiment:
X₃ is CR₃, where R₃ is selected from halo and haloalkyl;
X₁ is CR₁, where R₁ is H;
X₂ is CR₂, where R₂ is selected from halo and haloalkyl;
X₄ is CR₄, where R₄ is H.

In one particularly preferred embodiment:
X₃ is CR₃, where R₃ is selected from halo and haloalkyl;
X₁ is CR₁, where R₁ is H;
X₂ is CR₂, where R₂ is H;
X₄ is CR₄, where R₄ is selected from halo and haloalkyl.

In one particularly preferred embodiment:
X₃ is CR₃, where R₃ is selected from Cl, F and CF₃, even more preferably, Cl and CF₃;
X₁ is CR₁, where R₁ is H;
X₂ is CR₂, where R₂ is selected from H and Cl;
X₄ is CR₄, where R₄ is selected from H and Cl;
wherein at least one of R₂ and R₄ is other than H.

In one particularly preferred embodiment:
X₃ is CR₃, where R₃ is selected from Cl, F and CF₃, even more preferably, Cl and CF₃;
X₁ is CR₁, where R₁ is H;
X₂ is CR₂, where R₂ is Cl;
X₄ is CR₄, where R₄ is H.

In one particularly preferred embodiment:
X₃ is CR₃, where R₃ is selected from Cl, F and CF₃, even more preferably, Cl and CF₃;
X₁ is CR₁, where R₁ is H;
X₂ is CR₂, where R₂ is H;
X₄ is CR₄, where R₄ is Cl.

In one preferred embodiment of the invention, for compounds of formula (Ic), X₄ is N, X₁ is CR₁ and X₂ is CR₂. Preferably, for this embodiment, R₁ and R₂ are each independently selected from H and halo, more preferably, H and Cl.

In one preferred embodiment of the invention, for compounds of formula (Ic), X₁ is N, X₂ is CR₂ and X₄ is CR₄. Preferably, for this embodiment, R₂ and R₄ are both H.

In one preferred embodiment of the invention, for compounds of formula (Ic), X₂ is N, X₁ is CR₁ and X₄ is CR₄. Preferably, for this embodiment, R₁ and R₄ are each independently selected from H and halo, more preferably, H and Cl.

In one preferred embodiment, the compound is of formula (Ib) wherein n is 0, and X₁-X₄ form a 5-membered heteroaromatic group containing at least one nitrogen atom, said heteroaromatic group being optionally substituted by one or more substituents selected from halo, CN, alkyl, alkoxy, haloalkyl, aryl, heteroaryl, heterocycloalkyl, O-heterocycloalkyl, cycloalkyl, halocycloalkyl, hydroxycycloalkyl, O-cycloalkyl and O-aryl.

In one preferred embodiment, the compound is of formula (Ib) wherein n is 0, and X₁-X₄ form a 5-membered heteroaromatic group selected from oxadiazoyl, thiadiazolyl, imidazolyl, pyrrolyl, pyrazolyl, diazolyl, triazolyl, isoxazolyl, isothiazolyl, tetrazolyl, oxazolyl, and thiazolyl, and wherein said heteroaromatic group is optionally substituted by one or more substituents selected from halo, CN, alkyl, alkoxy, haloalkyl, aryl, heteroaryl, heterocycloalkyl, O-heterocycloalkyl, cycloalkyl, halocycloalkyl, hydroxycycloalkyl, O-cycloalkyl and O-aryl.

In one preferred embodiment, the compound is of formula (Ib) wherein n is 0, and X₁-X₄ form a 5-membered heteroaromatic group selected 1H-imidazol-5-yl, 1H-imidazol-4-yl, 1H-imidazol-2-yl, 1H-pyrrol-2-yl, 1H-pyrrol-3-yl, 1H-pyrrol-4-yl, 1H-pyrazol-5-yl, 1H-pyrazol-3-yl, 1H-pyrazol-4-yl, oxazol-2-yl, oxazol-4-yl, oxazol-5-yl, 1H-1,2,4-triazol-3-yl, 1H-1,2,4-triazol-5-yl, 1H-1,2,3-triazol-4-yl, 1H-1,2,3-triazol-5-yl, thiazol-5-yl, thiazol-4-yl, thiazol-2-yl, 1H-tetrazolyl, 2H-tetrazolyl, oxazol-5-yl, oxazol-4-yl, oxazol-2-yl, isoxazol-3-yl, isoxazol-4-yl, isoxazol-5-yl, isothiazol-3-yl, isothiazol-4-yl, isothiazol-5-yl, 1,3,4-oxadiazol-2-yl, 1,3,4-oxadiazol-5-yl, 1,2,5-oxadiazol-3-yl, 1,2,5-oxadiazol-4-yl, 1,2,3-oxadiazol-4-yl, 1,2,3-oxadiazol-5-yl, 1,2,4-oxadiazol-3-yl, 1,2,4-oxadiazol-5-yl, each of which is optionally substituted by one or more substituents selected from alkyl, halo, CN, alkoxy and haloalkyl.

In one highly preferred embodiment, the 5-membered heteroaromatic group is selected from 1,2,4-oxadiazol-3-yl, 1,2,5-oxadiazol-3-yl and 1,3,4-thiadiazol-2-yl, each of which is optionally substituted by one or more substituents selected from alkyl, halo, CN, alkoxy and haloalkyl.

In one preferred embodiment, the compound is of formula (Id), or a pharmaceutically acceptable salt or solvate thereof,

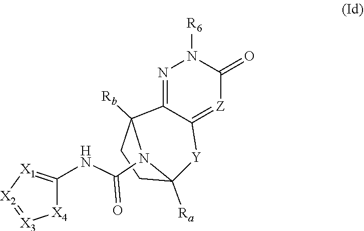

X₁-X₄ form a heteroaromatic group containing at least one nitrogen atom, wherein:
   X₁ is N or CR₁;
   X₂ is N or CR₂;
   X₃ is N, O, or CR₃;
   X₄ is selected from NR_{15″}, O and S, where R_{15″} is H, alkyl or haloalkyl; and
   R₁-R₃ are each independently selected from H, halo, CN, alkoxyl, alkyl, haloalkyl, aryl, heteroaryl and O-aryl, more preferably, H, halo, CN, alkoxyl, alkyl and haloalkyl; and
Z, Y, Rₐ, R_{b}, and R₆ are as defined above.

Preferred definitions for R₆, Rₐ, R_{b}, Y and Z as defined above apply equally to compounds of formula (Id).

In one preferred embodiment, the compound is of formula (Id), wherein X₁ is N, X₂ is N, X₃ is CR³ and X⁴ is S.

Preferably, for compounds of formula (Id), Z is CH.

In one preferred embodiment, the compound is of formula (Ih), or a pharmaceutically acceptable salt or solvate thereof,

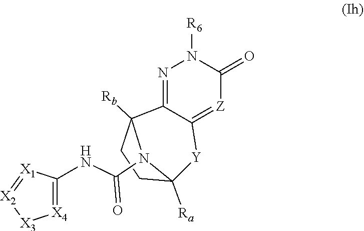

wherein:
X₁-X₄ form a heteroaromatic group containing at least one nitrogen atom, wherein:
   X₁ is N or CR₁;
   X₂ is N or CR₂;
   X₃ is selected from NR_{15″}, O and S, where R_{15″} is H, alkyl or haloalkyl;
   X₄ is N or CR₄; and
   R₁, R₂ and R₄ are each independently selected from H, halo, CN, alkoxyl, alkyl, haloalkyl, aryl, heteroaryl and O-aryl, more preferably, H, halo, CN, alkoxyl, alkyl and haloalkyl.

Preferred definitions for R₆, Rₐ, R_{b}, Y and Z as defined above apply equally to compounds of formula (Ih).

In one preferred embodiment, the compound is of formula (Ih), wherein X₁ is CR₁, X₂ is N, X₃ is O and X₄ is N; or X₁ is N, X₂ is CR₂, X₃ is O and X₄ is N.

In one preferred embodiment, ring B is an optionally substituted bicyclic heteroaromatic group containing at least one nitrogen atom, preferably, an optionally substituted 9- or 10-membered bicyclic heteroaromatic group containing at least one nitrogen atom.

In one preferred embodiment, ring B is a bicyclic heteroaromatic group selected from benzimidazolyl, pyrazolopyridinyl, indolyl, indolizinyl, isoindolyl, indazolyl, quinolinyl, isoquinolinyl, cinnolinyl, phthalazinyl, quinazolinyl, quinoxalinyl and naphthyridinyl, and wherein said bicyclic heteroaromatic group is optionally substituted by one or more substituents selected from halo, CN, alkyl, alkoxy, haloalkyl, aryl, heteroaryl and O-aryl.

In one highly preferred embodiment, B is a quinolin-6-yl group or a 2H-pyrazolo[3,4-c]pyridin-5-yl group, each of which is optionally substituted by one or more substituents selected from halo, CN, alkyl, alkoxy and haloalkyl.

In one preferred embodiment, the compound is of formula (Ii), or a pharmaceutically acceptable salt or solvate thereof,

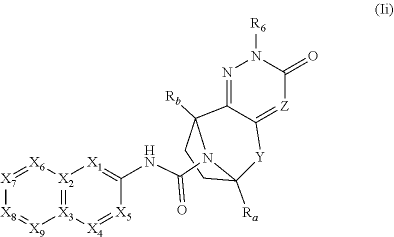

wherein X₂ and X₃ are both C, one, two or three of X₁, X₄, X₅—X₉ are N and the rest are selected from C—H, C-alkyl and C-haloalkyl, wherein Y, Z, R₆, Rₐ, R_{b} are as defined above.

Preferred definitions for R₆, Rₐ, R_{b}, Y and Z as defined above apply equally to compounds of formula (Ii).

In one preferred embodiment, the compound is of formula (Ii), wherein X₉ is N, X₂ and X₃ are both C, X₁, X₄, X₅ X₆ and X₇ are CH, and X₈ is C-haloalkyl more preferably, CF₃.

In one preferred embodiment, B is a bicyclic heteroaromatic group containing at least one nitrogen atom which is selected from the following:

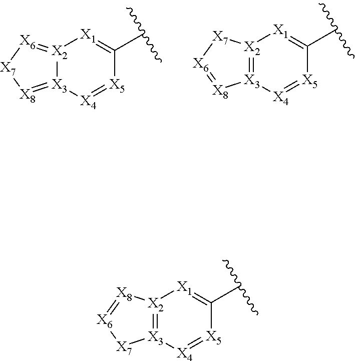

wherein:
X₂ and X₃ are both C;
X₇ is selected from O, S and NR_{15″}, where R_{15″} is H, alkyl or haloalkyl; and one, two or three of X₁, X₄, X₅, X₆ and X₈ are N and the rest are selected from C—H, C-alkyl and C-haloalkyl.

In one preferred embodiment, B is a heteroaromatic group containing at least one nitrogen atom which is of formula:

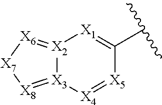

wherein X₅, X₈ are N, X₂ and X₃ are both C, X₁, X₄, X₅ and X₆ are CH, and X₇ is N-haloalkyl, more preferably N—CHF₂.

In one preferred embodiment for the compounds described herein, ring B is preferably selected from the following groups:

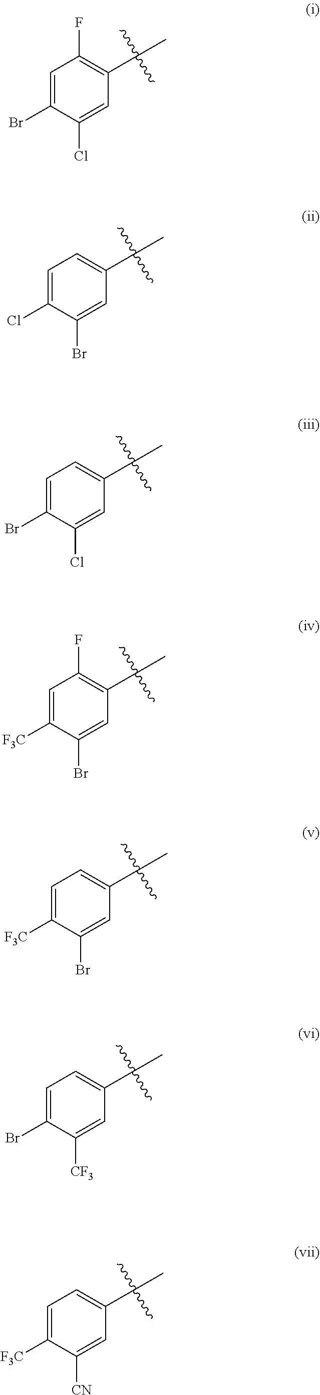

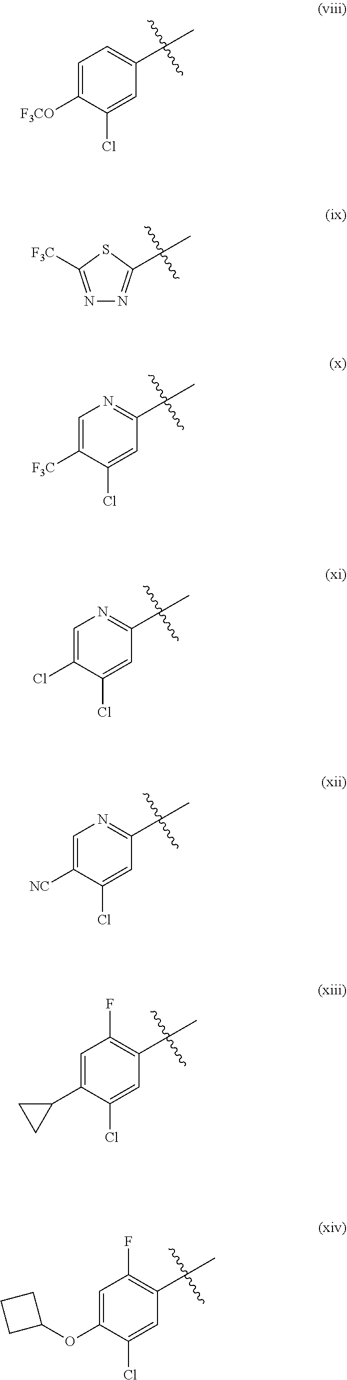

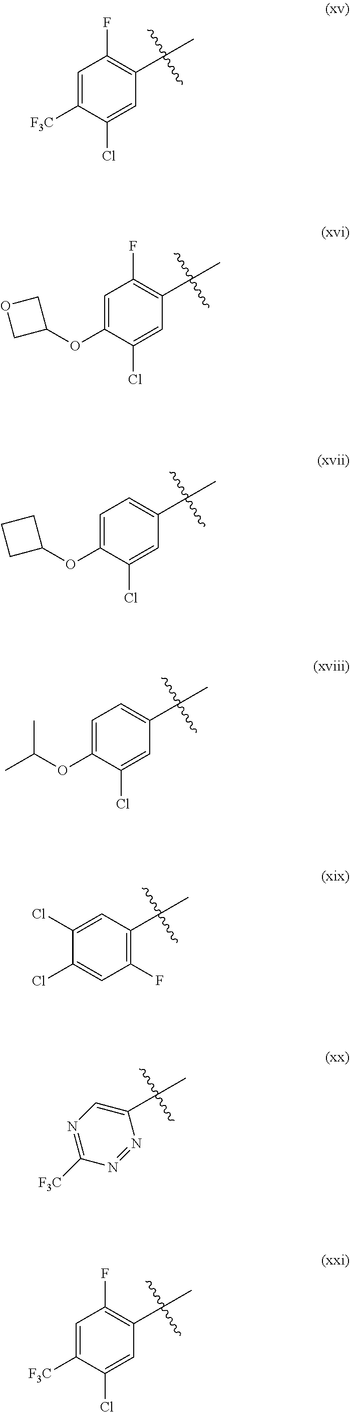

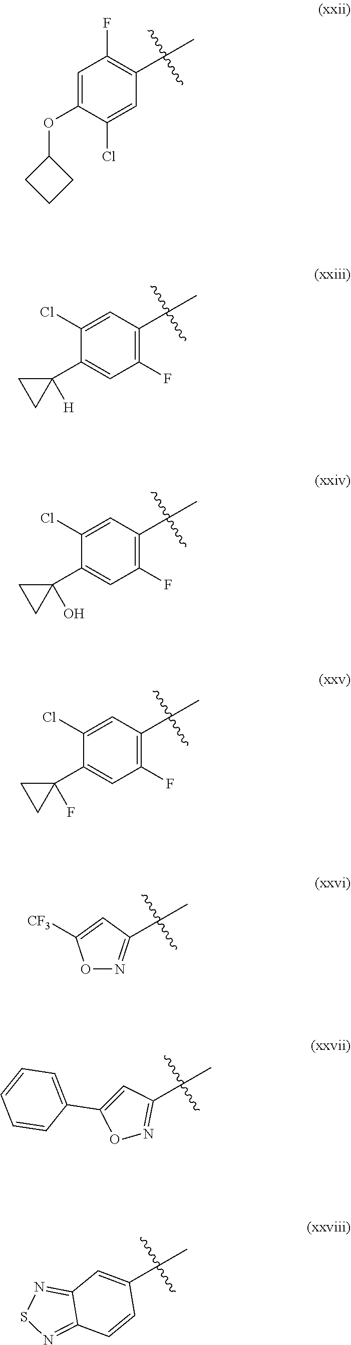

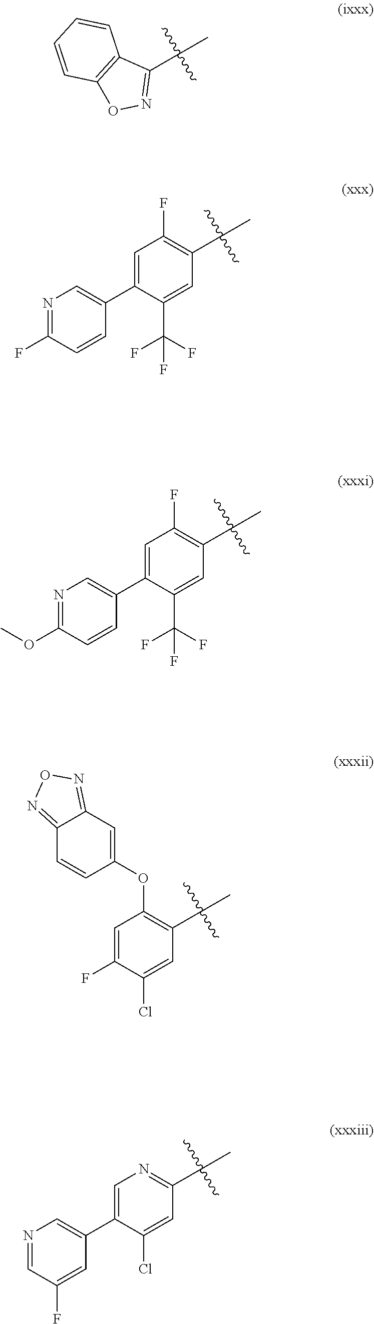 [SMILES: *c1noc2ccccc12 ; *c1cc(C(F)(F)F)c(-c2ccc(F)nc2)cc1F ; *c1cc(C(F)(F)F)c(-c2ccc(OC)nc2)cc1F ; *c1cc(Cl)c(F)cc1Oc1ccc2nonc2c1 ; *c1cc(Cl)c(-c2cncc(F)c2)cn1]

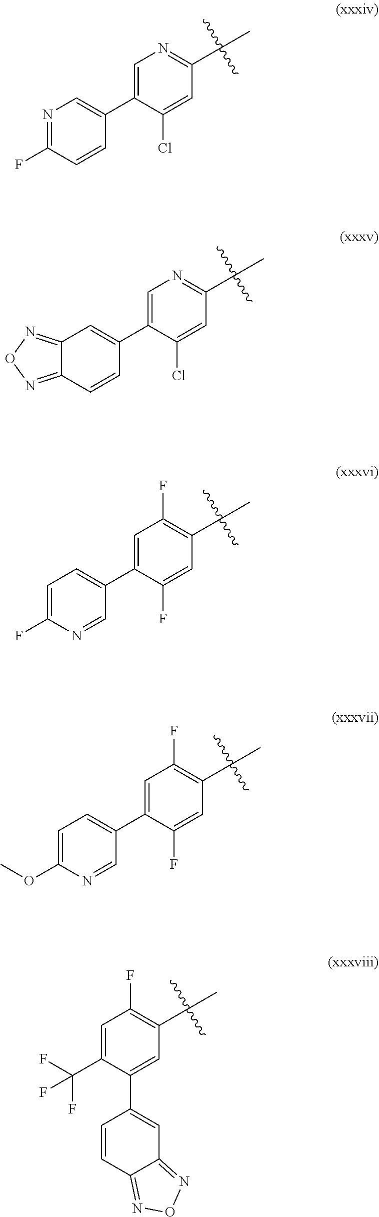 [SMILES: *c1cc(Cl)c(-c2ccc(F)nc2)cn1 ; *c1cc(Cl)c(-c2ccc3nonc3c2)cn1 ; *c1cc(F)c(-c2ccc(F)nc2)cc1F ; *c1cc(F)c(-c2ccc(OC)nc2)cc1F ; *c1cc(-c2ccc3nonc3c2)c(C(F)(F)F)cc1F]

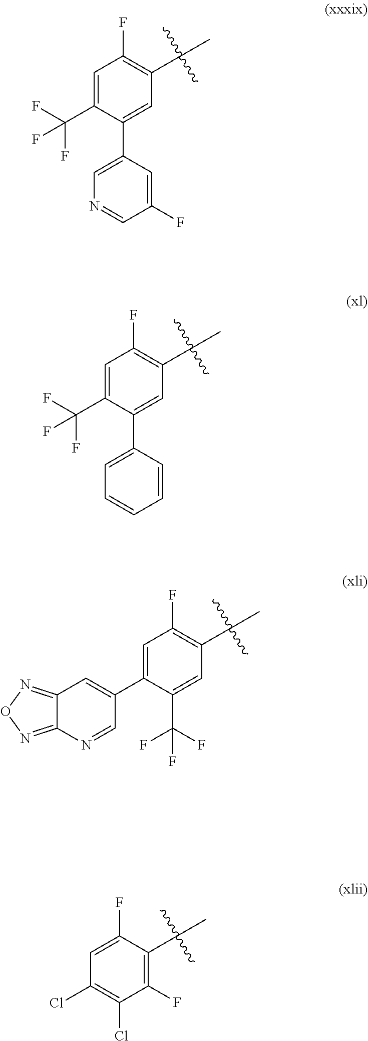 [SMILES: *c1cc(-c2cncc(F)c2)c(C(F)(F)F)cc1F ; *c1cc(-c2ccccc2)c(C(F)(F)F)cc1F ; *c1cc(C(F)(F)F)c(-c2cnc3nonc3c2)cc1F ; *c1c(F)cc(Cl)c(Cl)c1F]

In one preferred embodiment, ring B is selected from groups (i)-(xxviii) above.

In one preferred embodiment, ring B is selected from groups (i)-(xxviii) above, Z is CH, Rₐ, R_{b} and R₆ are H, and Y is CH₂.

In another preferred embodiment, ring B is:

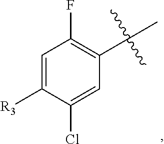

wherein R₃ is selected from:

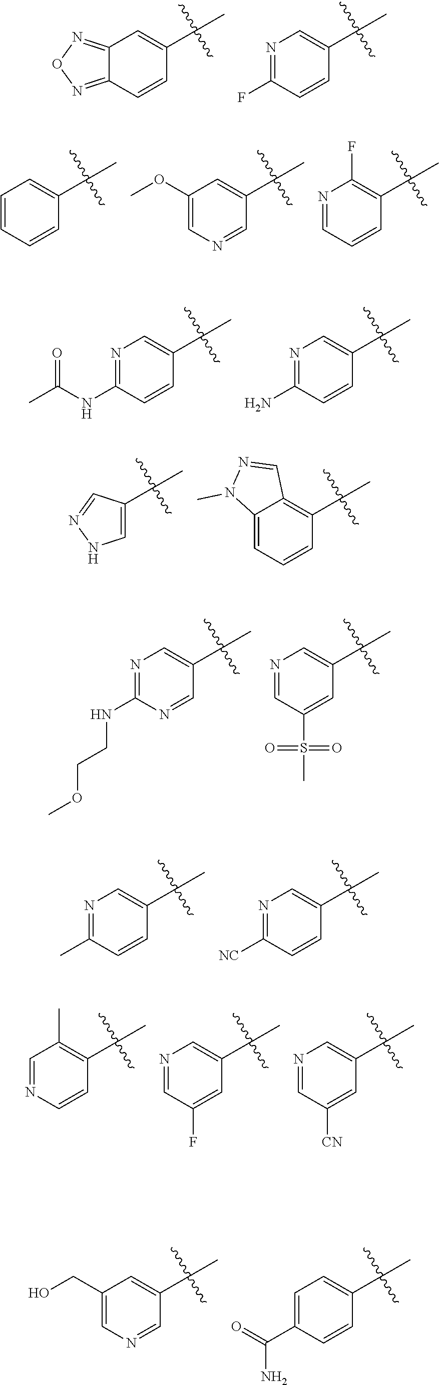

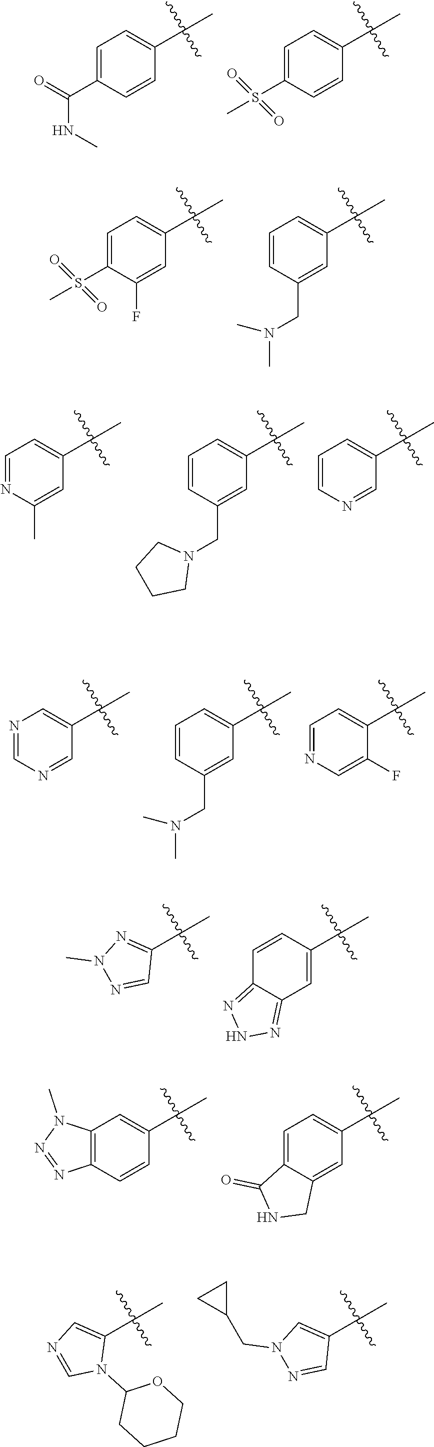

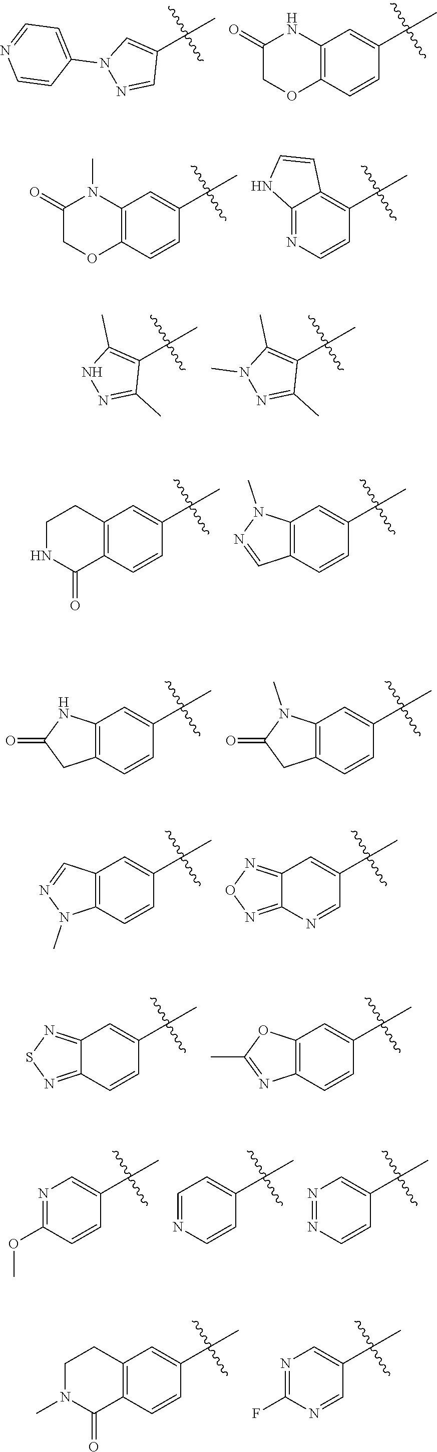

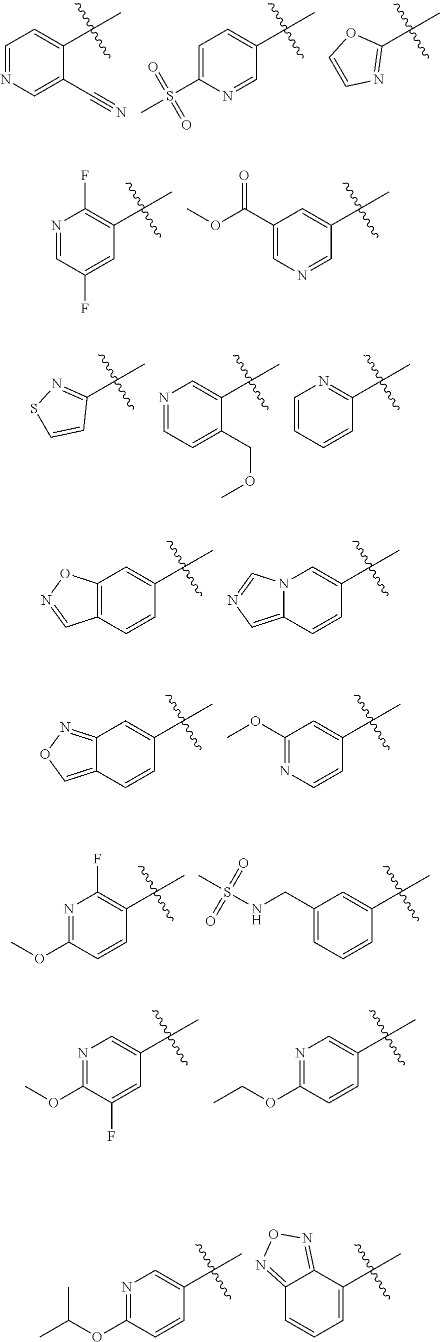

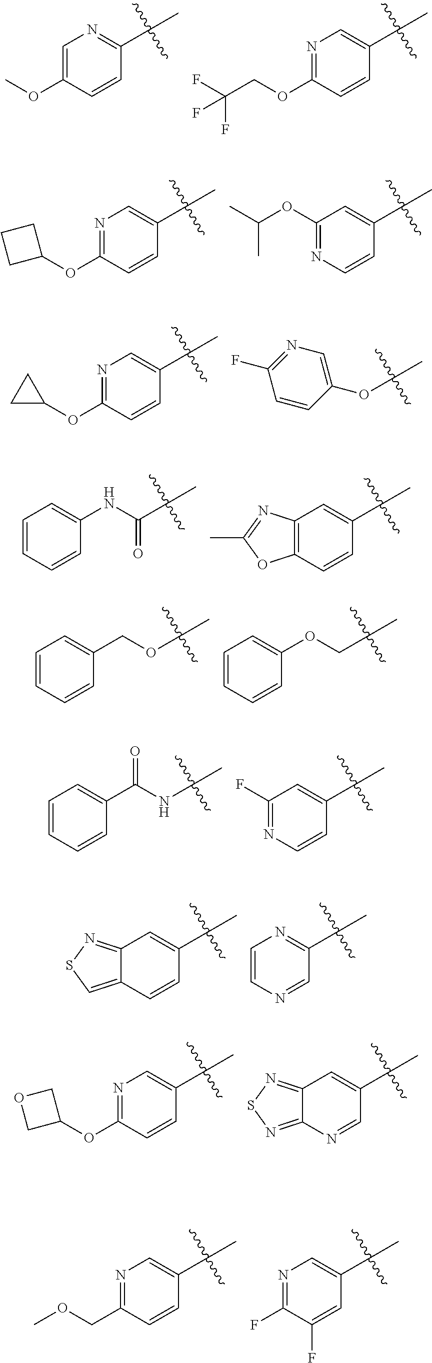

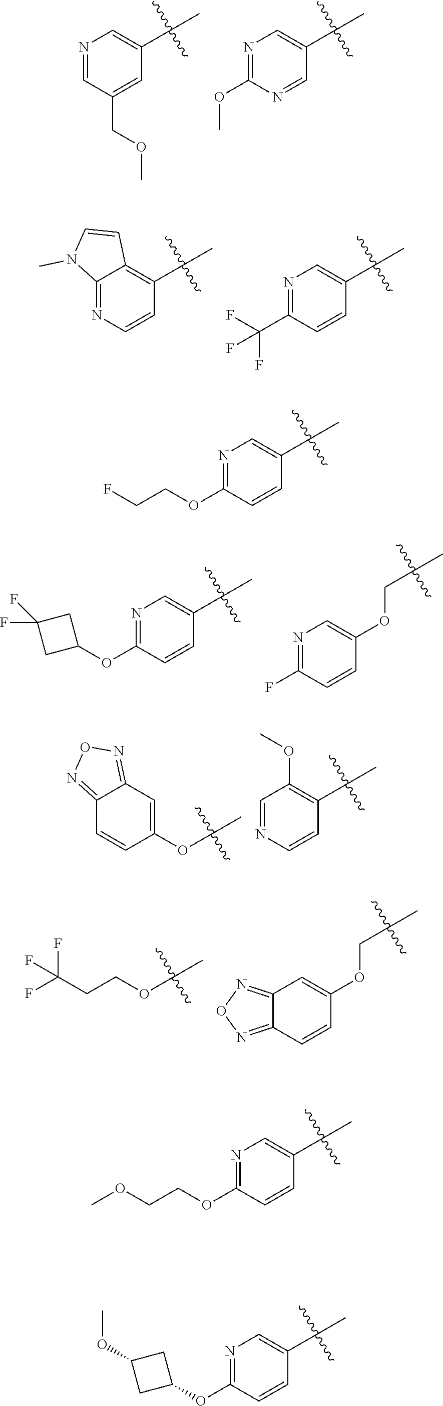

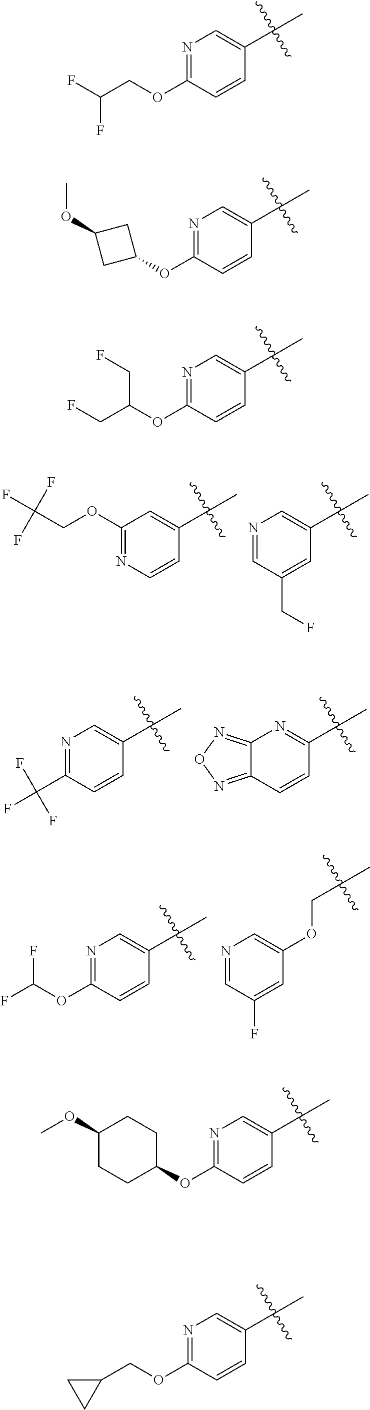

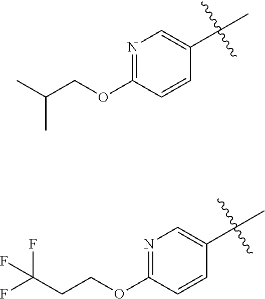 [SMILES: *c1ccc(OCC(C)C)nc1 ; *c1ccc(OCCC(F)(F)F)nc1]

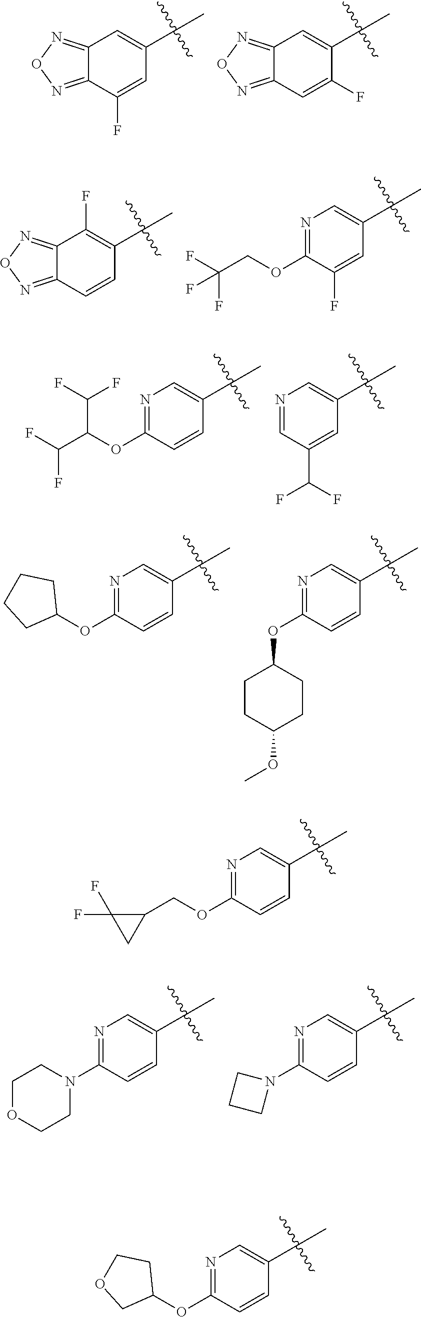

In one preferred embodiment, ring B is selected from the groups listed above, Z is CH, Rₐ, R_{b} and R₆ are H, and Y is CH₂.

In one particularly preferred embodiment, the compound is of formula (I.1):

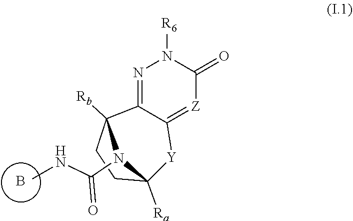

wherein B, Y, Rₐ, R_{b}, Z and R₆ are as defined above.

In one preferred embodiment, the compound is in enantiomerically pure form. In one preferred embodiment, the compound is in the form of a mixture that is enantiomerically enriched with a compound of formula (I.1).

In another embodiment, the compound is of formula (I.2):

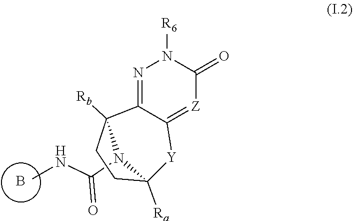

wherein B, Y, Rₐ, R_{b}, Z and R₆ are as defined above.

Enantiomeric forma (1.1) and (1.2) apply equally to all of the various subformulae described herein.

In one preferred embodiment, the compound is in enantiomerically pure form. In one preferred embodiment, the compound is in the form of a mixture that is enantiomerically enriched with a compound of formula (I.2).

In one preferred embodiment, the compound is in the form of a mixture comprising a compound of formula (I.1) and its corresponding enantiomer of formula (I.2). In one preferred embodiment, the mixture is a racemic mixture, i.e. a 50:50 mixture of a compound of formula (I.1) and its corresponding enantiomer of formula (I.2).

Racemic mixtures can be used to prepare enantiomerically pure compounds of formula (I.1) or (I.2) by separating the compounds of formula (I.1) or (I.2) by standard methods, for example by chemical resolution using optically active acid or by the use of column chromatography or reverse-phase column chromatography using a substantially optically active (or “chiral”) stationary phase as known to those skilled in the art. Racemic mixtures can also be used to prepare enantiomerically enriched mixtures of compounds of formula (I.1) or (I.2). Mixtures enriched with either a compound of formula (I.1) or (I.2) can also be obtained from the appropriate enantiomerically enriched precursors.

In one preferred embodiment of the invention, the compound is in the form of a mixture comprising enantiomers wherein the weight:weight ratio is at least approximately 2:1 or greater, preferably at least approximately 5:1 or greater, most preferably at least approximately 10:1 or greater in favour of the enantiomer that displays significant in vitro and/or in vivo activity (the eutomer).

In one particularly preferred embodiment, the compound is in the form of a mixture comprising a compound of formula (I.1) and its corresponding enantiomer of formula (I.2), wherein the weight:weight ratio of said compound of formula (I.1) to said compound of formula (I.2) is greater than 1.05:1, more preferably, greater than 2:1, even more preferably greater than 5:1, even more preferably greater than 10:1.

In one particularly preferred embodiment, the compound is in the form of a mixture comprising a compound of formula (I.1) and its corresponding enantiomer of formula (I.2), which is substantially enriched with said compound of formula (I.1).

In one embodiment, the compound is in the form of a mixture comprising a compound of formula (I.1) and its corresponding enantiomer of formula (I.2), wherein the weight:weight ratio of said compound of formula (I.2) to said compound of formula (I.1) is greater than 1.05:1, more preferably, greater than 2:1, even more preferably greater than 5:1, even more preferably greater than 10:1.

In one embodiment, the compound is in the form of a mixture comprising a compound of formula (I.1) and its corresponding enantiomer of formula (I.2), which is substantially enriched with said compound of formula (I.2).

In one preferred embodiment, the compound is selected from the following:

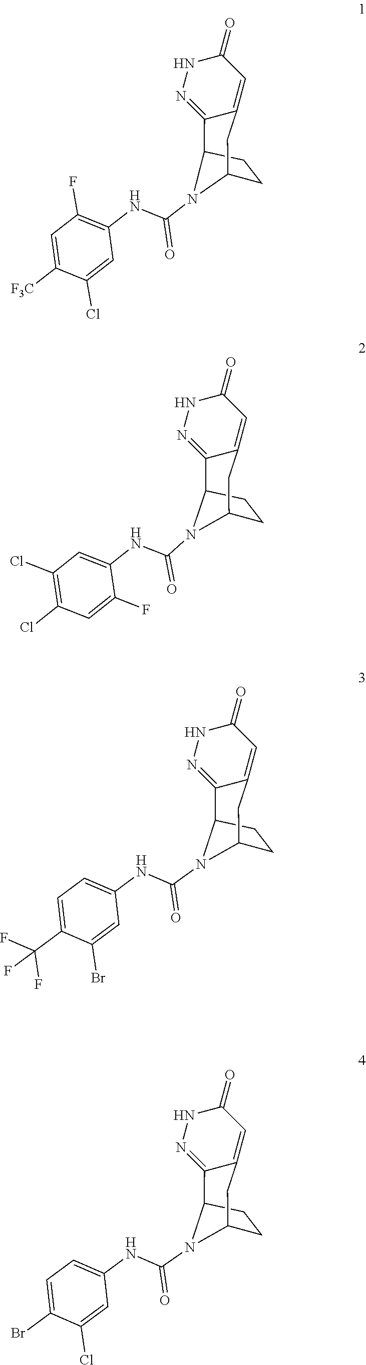 [SMILES: O=C(Nc1cc(Cl)c(C(F)(F)F)cc1F)N1C2CCC1c1n[nH]c(=O)cc1C2 ; O=C(Nc1cc(Cl)c(Cl)cc1F)N1C2CCC1c1n[nH]c(=O)cc1C2 ; O=C(Nc1ccc(C(F)(F)F)c(Br)c1)N1C2CCC1c1n[nH]c(=O)cc1C2 ; O=C(Nc1ccc(Br)c(Cl)c1)N1C2CCC1c1n[nH]c(=O)cc1C2]

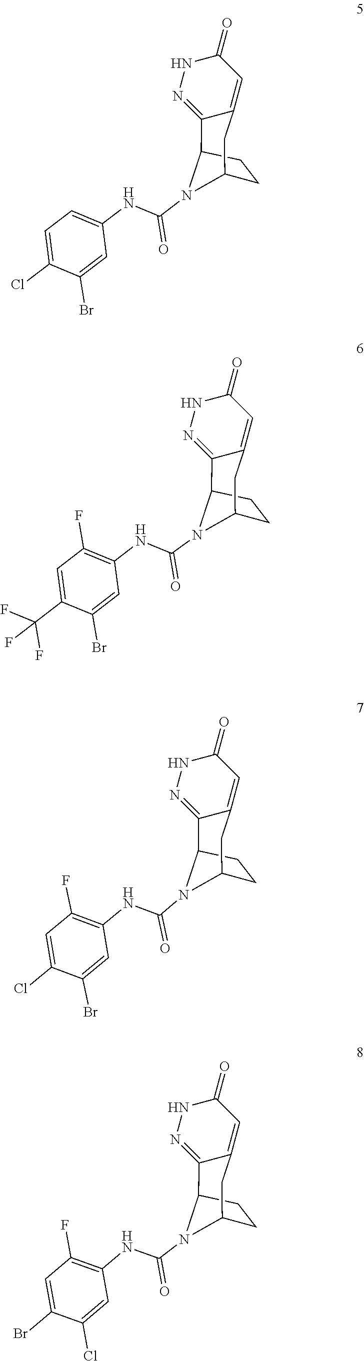 [SMILES: O=C(Nc1ccc(Cl)c(Br)c1)N1C2CCC1c1n[nH]c(=O)cc1C2 ; O=C(Nc1cc(Br)c(C(F)(F)F)cc1F)N1C2CCC1c1n[nH]c(=O)cc1C2 ; O=C(Nc1cc(Br)c(Cl)cc1F)N1C2CCC1c1n[nH]c(=O)cc1C2 ; O=C(Nc1cc(Cl)c(Br)cc1F)N1C2CCC1c1n[nH]c(=O)cc1C2]

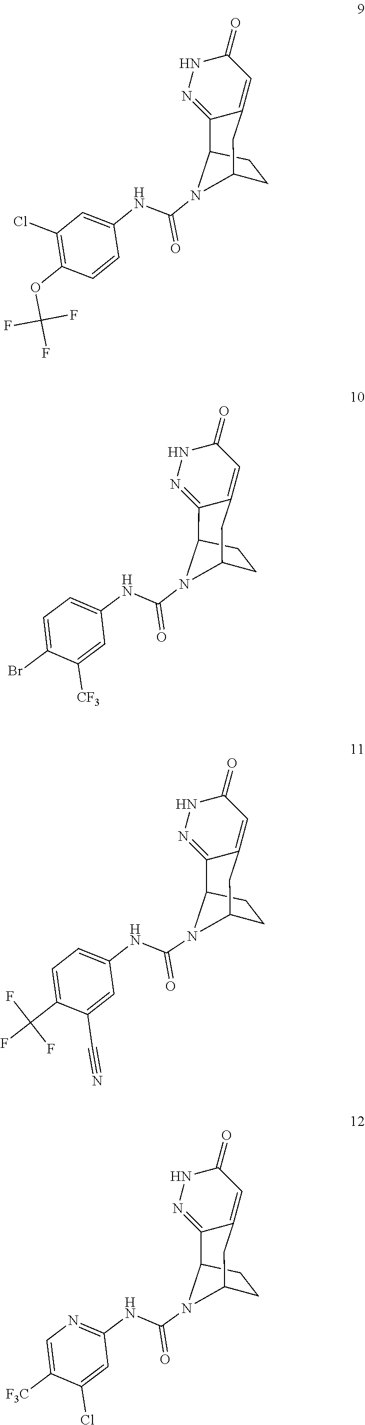 [SMILES: O=C(Nc1ccc(OC(F)(F)F)c(Cl)c1)N1C2CCC1c1n[nH]c(=O)cc1C2 ; O=C(Nc1ccc(Br)c(C(F)(F)F)c1)N1C2CCC1c1n[nH]c(=O)cc1C2 ; N#Cc1cc(NC(=O)N2C3CCC2c2n[nH]c(=O)cc2C3)ccc1C(F)(F)F ; O=C(Nc1cc(Cl)c(C(F)(F)F)cn1)N1C2CCC1c1n[nH]c(=O)cc1C2]

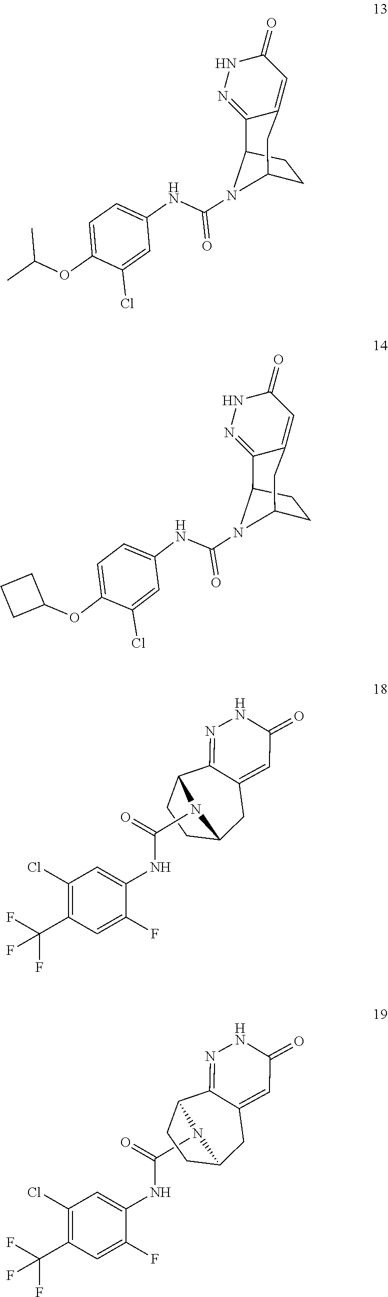 [SMILES: CC(C)Oc1ccc(NC(=O)N2C3CCC2c2n[nH]c(=O)cc2C3)cc1Cl ; O=C(Nc1ccc(OC2CCC2)c(Cl)c1)N1C2CCC1c1n[nH]c(=O)cc1C2 ; O=C(Nc1cc(Cl)c(C(F)(F)F)cc1F)N1[C@H]2CC[C@@H]1c1n[nH]c(=O)cc1C2 ; O=C(Nc1cc(Cl)c(C(F)(F)F)cc1F)N1[C@@H]2CC[C@H]1c1n[nH]c(=O)cc1C2]

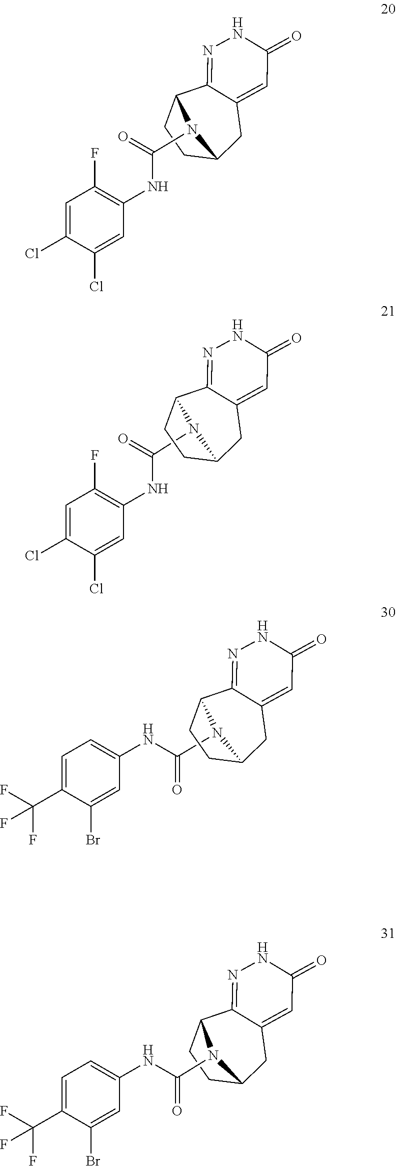 [SMILES: O=C(Nc1cc(Cl)c(Cl)cc1F)N1[C@H]2CC[C@@H]1c1n[nH]c(=O)cc1C2 ; O=C(Nc1cc(Cl)c(Cl)cc1F)N1[C@@H]2CC[C@H]1c1n[nH]c(=O)cc1C2 ; O=C(Nc1ccc(C(F)(F)F)c(Br)c1)N1[C@@H]2CC[C@H]1c1n[nH]c(=O)cc1C2 ; O=C(Nc1ccc(C(F)(F)F)c(Br)c1)N1[C@H]2CC[C@@H]1c1n[nH]c(=O)cc1C2]

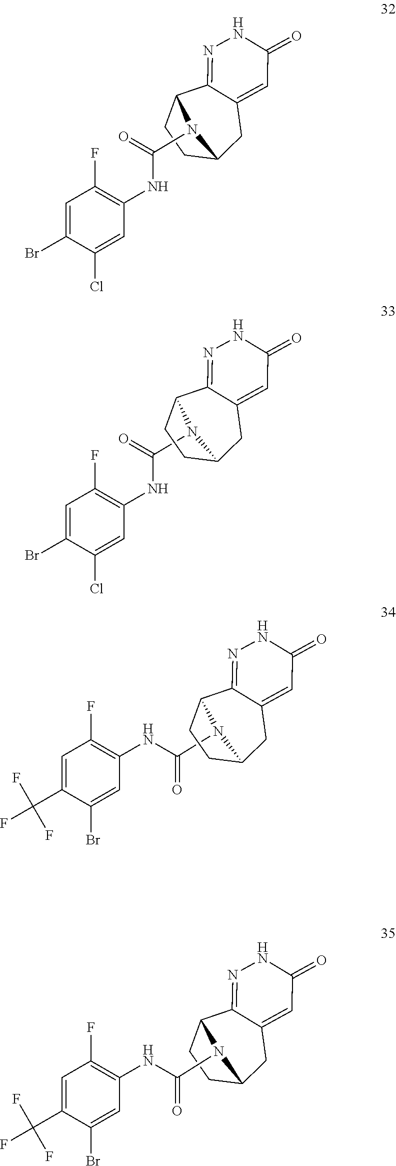 [SMILES: O=C(Nc1cc(Cl)c(Br)cc1F)N1[C@H]2CC[C@@H]1c1n[nH]c(=O)cc1C2 ; O=C(Nc1cc(Cl)c(Br)cc1F)N1[C@@H]2CC[C@H]1c1n[nH]c(=O)cc1C2 ; O=C(Nc1cc(Br)c(C(F)(F)F)cc1F)N1[C@@H]2CC[C@H]1c1n[nH]c(=O)cc1C2 ; O=C(Nc1cc(Br)c(C(F)(F)F)cc1F)N1[C@H]2CC[C@@H]1c1n[nH]c(=O)cc1C2]

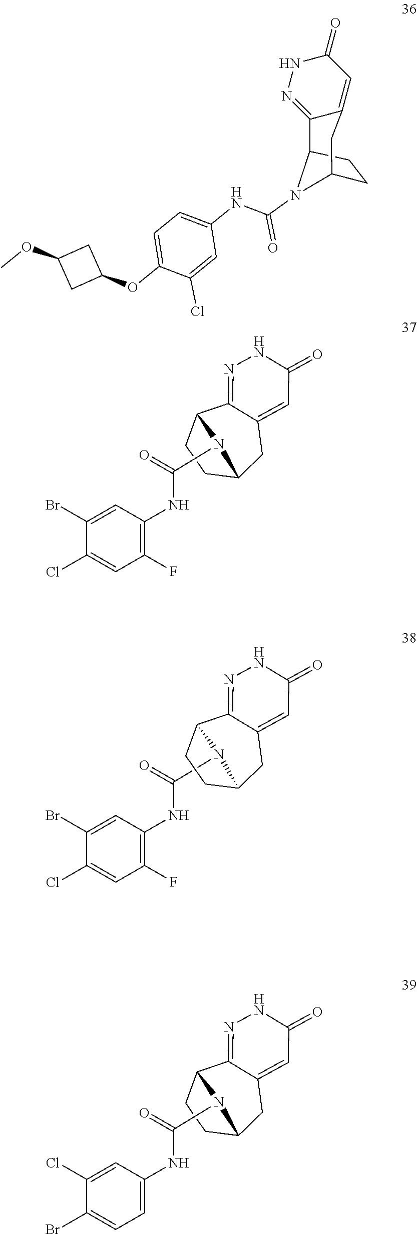 [SMILES: CO[C@H]1C[C@@H](Oc2ccc(NC(=O)N3C4CCC3c3n[nH]c(=O)cc3C4)cc2Cl)C1 ; O=C(Nc1cc(Br)c(Cl)cc1F)N1[C@H]2CC[C@@H]1c1n[nH]c(=O)cc1C2 ; O=C(Nc1cc(Br)c(Cl)cc1F)N1[C@@H]2CC[C@H]1c1n[nH]c(=O)cc1C2 ; O=C(Nc1ccc(Br)c(Cl)c1)N1[C@H]2CC[C@@H]1c1n[nH]c(=O)cc1C2]

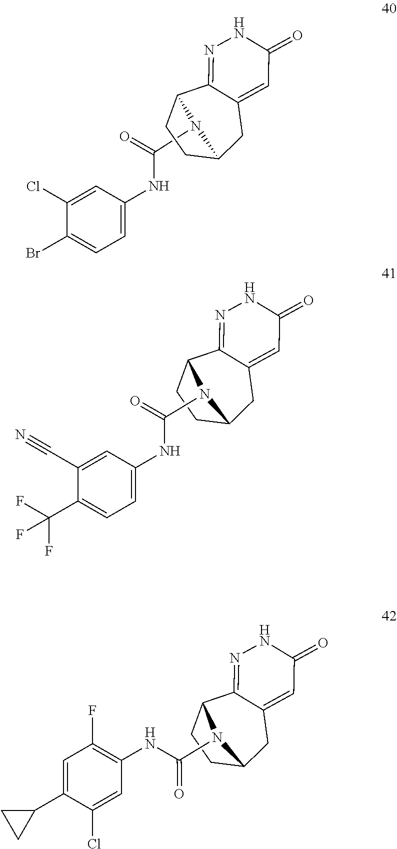 [SMILES: O=C(Nc1ccc(Br)c(Cl)c1)N1[C@@H]2CC[C@H]1c1n[nH]c(=O)cc1C2 ; N#Cc1cc(NC(=O)N2[C@H]3CC[C@@H]2c2n[nH]c(=O)cc2C3)ccc1C(F)(F)F ; O=C(Nc1cc(Cl)c(C2CC2)cc1F)N1[C@H]2CC[C@@H]1c1n[nH]c(=O)cc1C2]

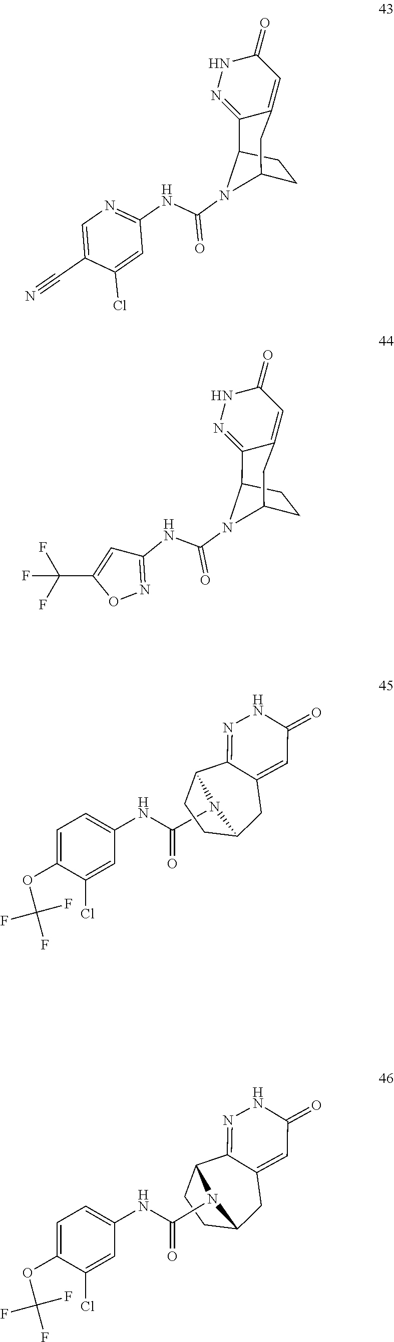 [SMILES: N#Cc1cnc(NC(=O)N2C3CCC2c2n[nH]c(=O)cc2C3)cc1Cl ; O=C(Nc1cc(C(F)(F)F)on1)N1C2CCC1c1n[nH]c(=O)cc1C2 ; O=C(Nc1ccc(OC(F)(F)F)c(Cl)c1)N1[C@@H]2CC[C@H]1c1n[nH]c(=O)cc1C2 ; O=C(Nc1ccc(OC(F)(F)F)c(Cl)c1)N1[C@H]2CC[C@@H]1c1n[nH]c(=O)cc1C2]

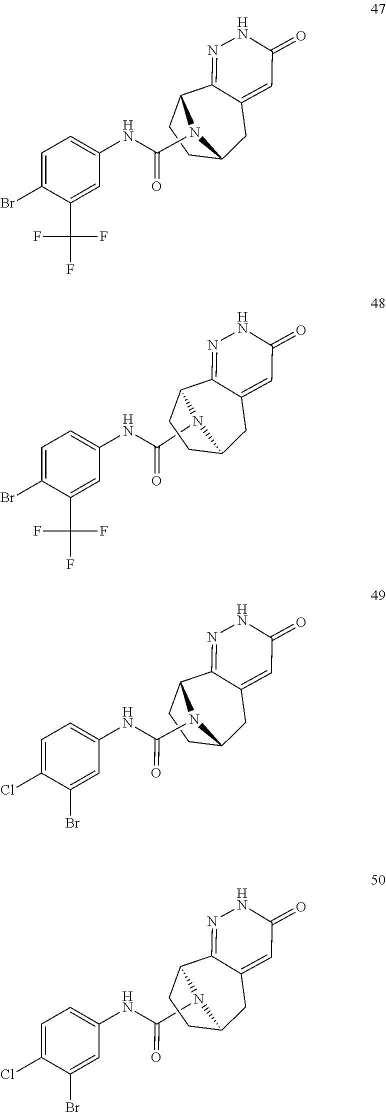 [SMILES: O=C(Nc1ccc(Br)c(C(F)(F)F)c1)N1[C@H]2CC[C@@H]1c1n[nH]c(=O)cc1C2 ; O=C(Nc1ccc(Br)c(C(F)(F)F)c1)N1[C@@H]2CC[C@H]1c1n[nH]c(=O)cc1C2 ; O=C(Nc1ccc(Cl)c(Br)c1)N1[C@H]2CC[C@@H]1c1n[nH]c(=O)cc1C2 ; O=C(Nc1ccc(Cl)c(Br)c1)N1[C@@H]2CC[C@H]1c1n[nH]c(=O)cc1C2]

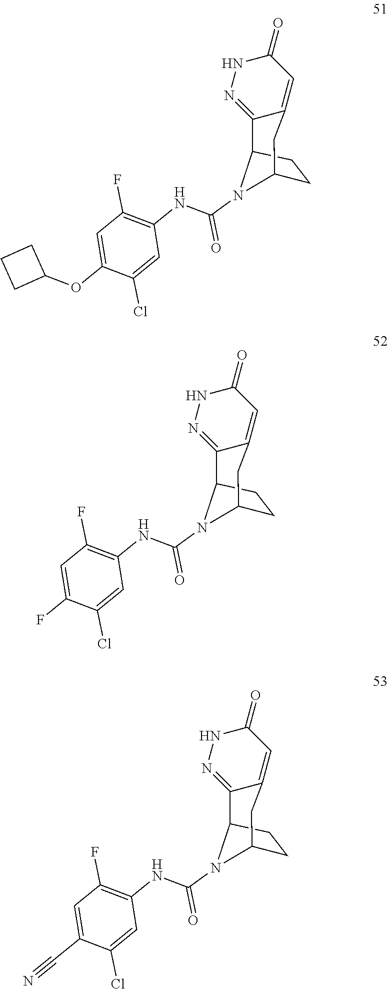 [SMILES: O=C(Nc1cc(Cl)c(OC2CCC2)cc1F)N1C2CCC1c1n[nH]c(=O)cc1C2 ; O=C(Nc1cc(Cl)c(F)cc1F)N1C2CCC1c1n[nH]c(=O)cc1C2 ; N#Cc1cc(F)c(NC(=O)N2C3CCC2c2n[nH]c(=O)cc2C3)cc1Cl]

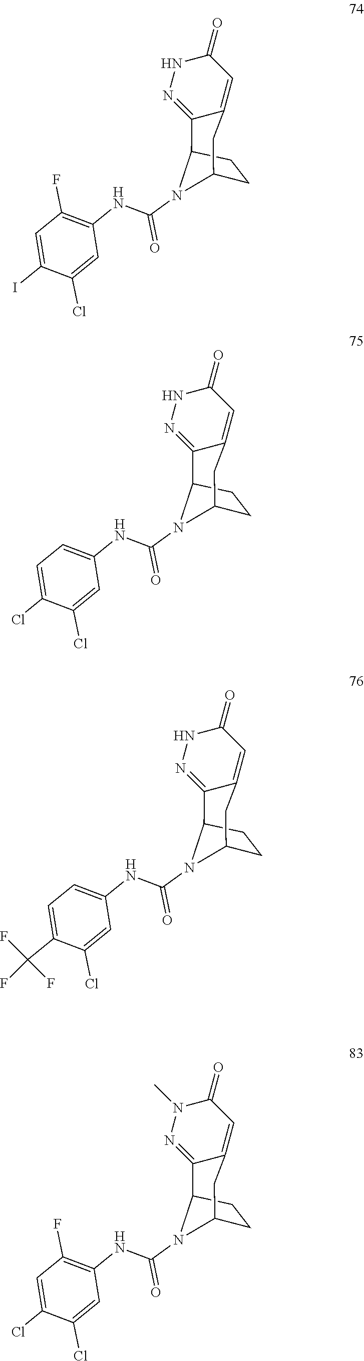 [SMILES: O=C(Nc1cc(Cl)c(I)cc1F)N1C2CCC1c1n[nH]c(=O)cc1C2 ; O=C(Nc1ccc(Cl)c(Cl)c1)N1C2CCC1c1n[nH]c(=O)cc1C2 ; O=C(Nc1ccc(C(F)(F)F)c(Cl)c1)N1C2CCC1c1n[nH]c(=O)cc1C2 ; Cn1nc2c(cc1=O)CC1CCC2N1C(=O)Nc1cc(Cl)c(Cl)cc1F]

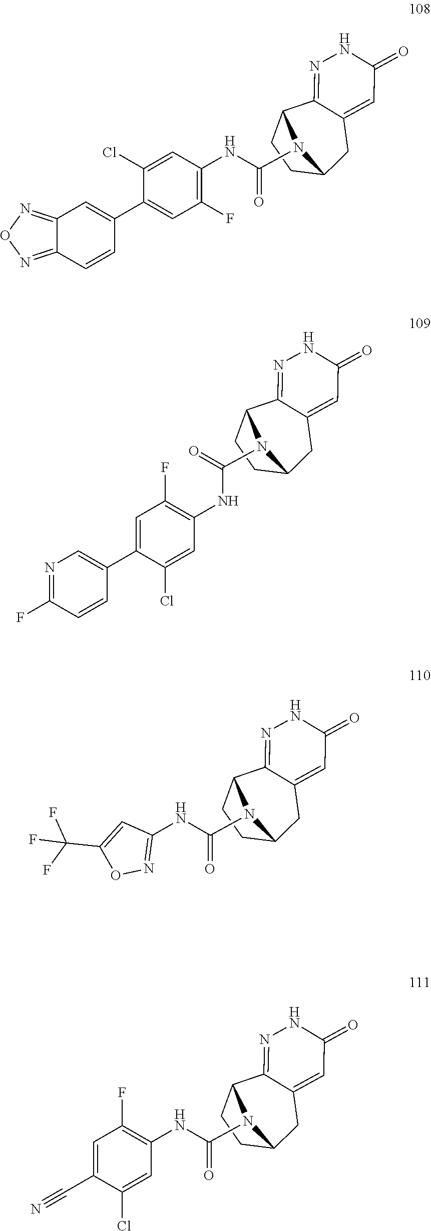 [SMILES: O=C(Nc1cc(Cl)c(-c2ccc3nonc3c2)cc1F)N1[C@H]2CC[C@@H]1c1n[nH]c(=O)cc1C2 ; O=C(Nc1cc(Cl)c(-c2ccc(F)nc2)cc1F)N1[C@H]2CC[C@@H]1c1n[nH]c(=O)cc1C2 ; O=C(Nc1cc(C(F)(F)F)on1)N1[C@H]2CC[C@@H]1c1n[nH]c(=O)cc1C2 ; N#Cc1cc(F)c(NC(=O)N2[C@H]3CC[C@@H]2c2n[nH]c(=O)cc2C3)cc1Cl]

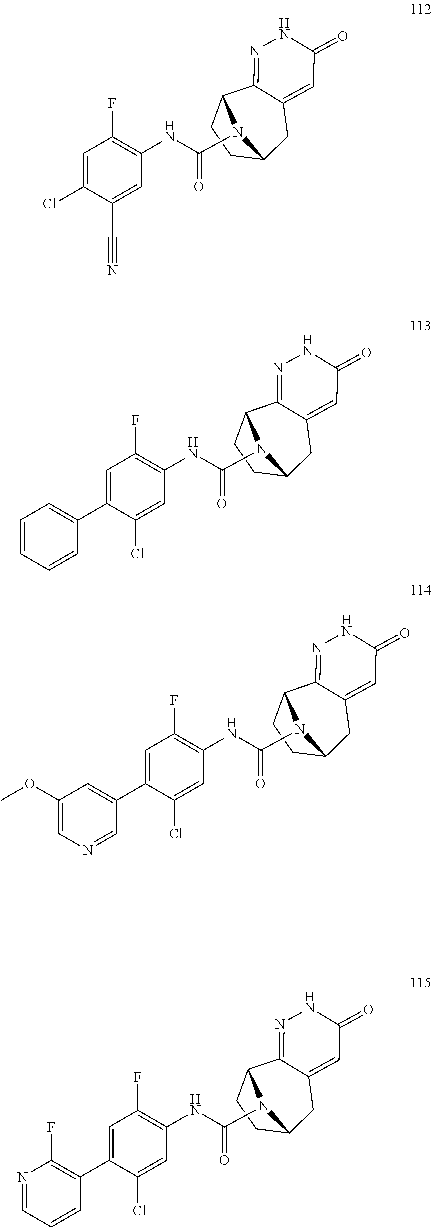 [SMILES: N#Cc1cc(NC(=O)N2[C@H]3CC[C@@H]2c2n[nH]c(=O)cc2C3)c(F)cc1Cl ; O=C(Nc1cc(Cl)c(-c2ccccc2)cc1F)N1[C@H]2CC[C@@H]1c1n[nH]c(=O)cc1C2 ; COc1cncc(-c2cc(F)c(NC(=O)N3[C@H]4CC[C@@H]3c3n[nH]c(=O)cc3C4)cc2Cl)c1 ; O=C(Nc1cc(Cl)c(-c2cccnc2F)cc1F)N1[C@H]2CC[C@@H]1c1n[nH]c(=O)cc1C2]

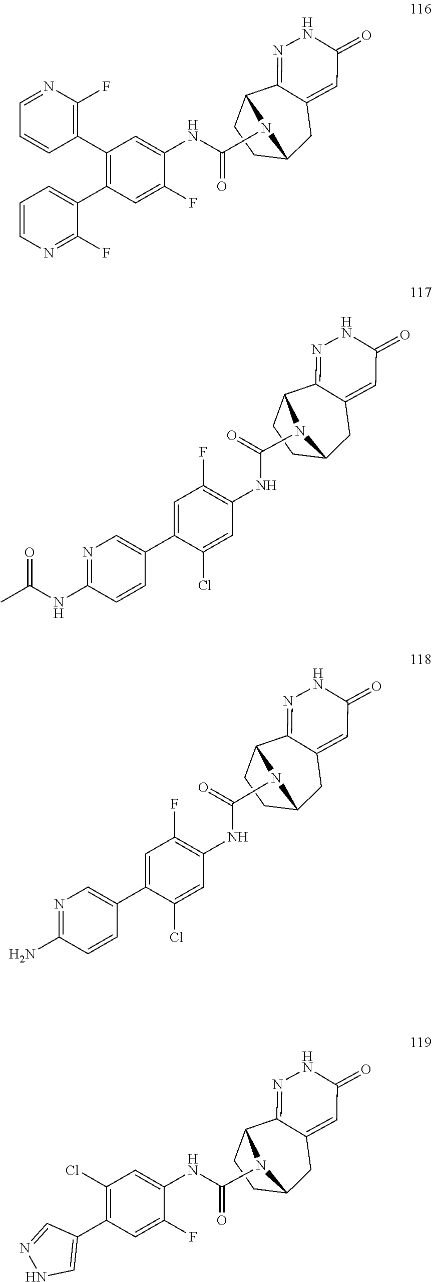 [SMILES: O=C(Nc1cc(-c2cccnc2F)c(-c2cccnc2F)cc1F)N1[C@H]2CC[C@@H]1c1n[nH]c(=O)cc1C2 ; CC(=O)Nc1ccc(-c2cc(F)c(NC(=O)N3[C@H]4CC[C@@H]3c3n[nH]c(=O)cc3C4)cc2Cl)cn1 ; Nc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2cn[nH]c2)cc1F)N1[C@H]2CC[C@@H]1c1n[nH]c(=O)cc1C2]

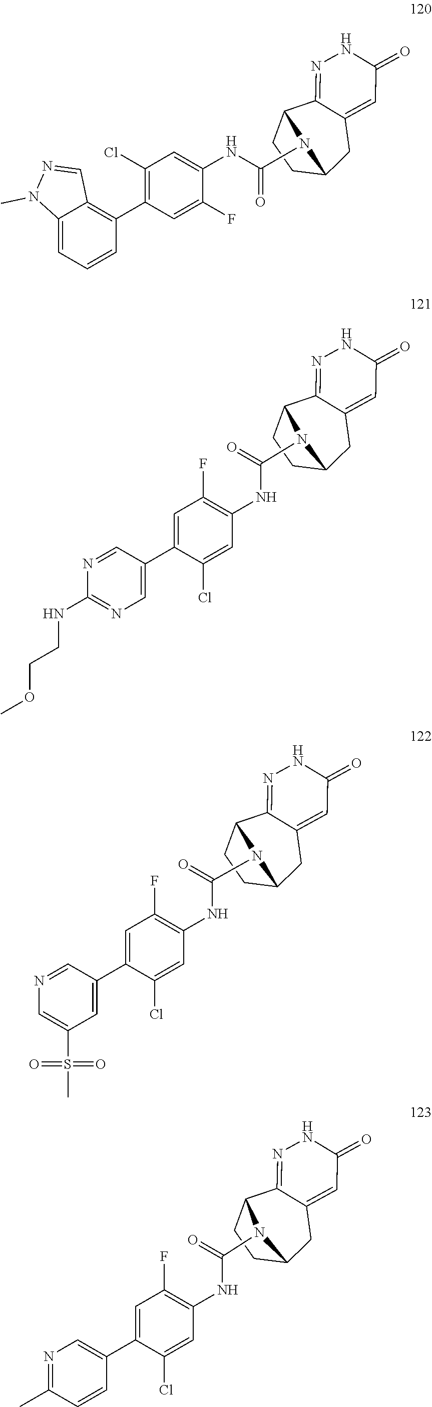 [SMILES: Cn1ncc2c(-c3cc(F)c(NC(=O)N4[C@H]5CC[C@@H]4c4n[nH]c(=O)cc4C5)cc3Cl)cccc21 ; COCCNc1ncc(-c2cc(F)c(NC(=O)N3[C@H]4CC[C@@H]3c3n[nH]c(=O)cc3C4)cc2Cl)cn1 ; CS(=O)(=O)c1cncc(-c2cc(F)c(NC(=O)N3[C@H]4CC[C@@H]3c3n[nH]c(=O)cc3C4)cc2Cl)c1 ; Cc1ccc(-c2cc(F)c(NC(=O)N3[C@H]4CC[C@@H]3c3n[nH]c(=O)cc3C4)cc2Cl)cn1]

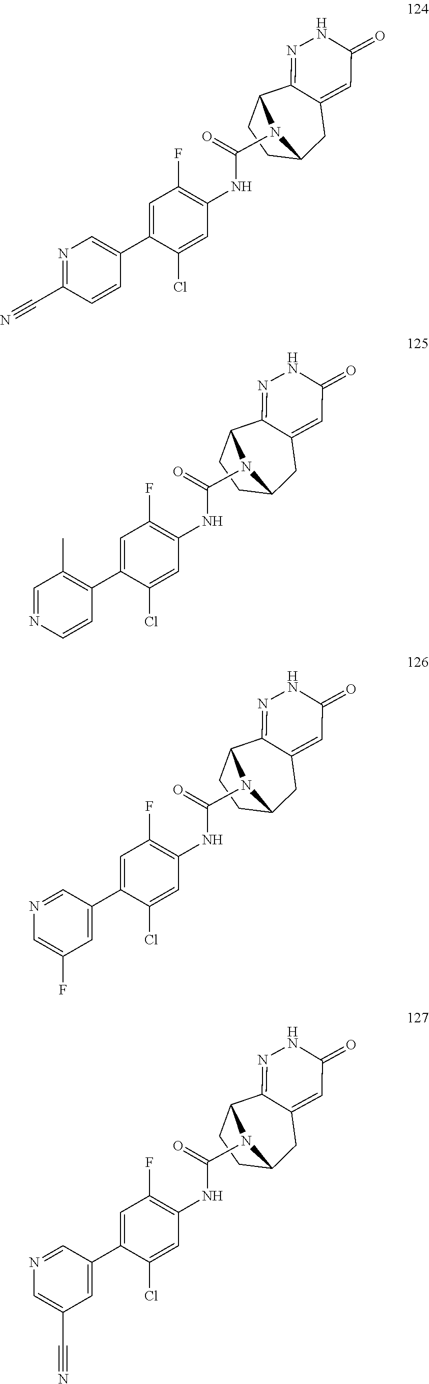 [SMILES: N#Cc1ccc(-c2cc(F)c(NC(=O)N3[C@H]4CC[C@@H]3c3n[nH]c(=O)cc3C4)cc2Cl)cn1 ; Cc1cnccc1-c1cc(F)c(NC(=O)N2[C@H]3CC[C@@H]2c2n[nH]c(=O)cc2C3)cc1Cl ; O=C(Nc1cc(Cl)c(-c2cncc(F)c2)cc1F)N1[C@H]2CC[C@@H]1c1n[nH]c(=O)cc1C2 ; N#Cc1cncc(-c2cc(F)c(NC(=O)N3[C@H]4CC[C@@H]3c3n[nH]c(=O)cc3C4)cc2Cl)c1]

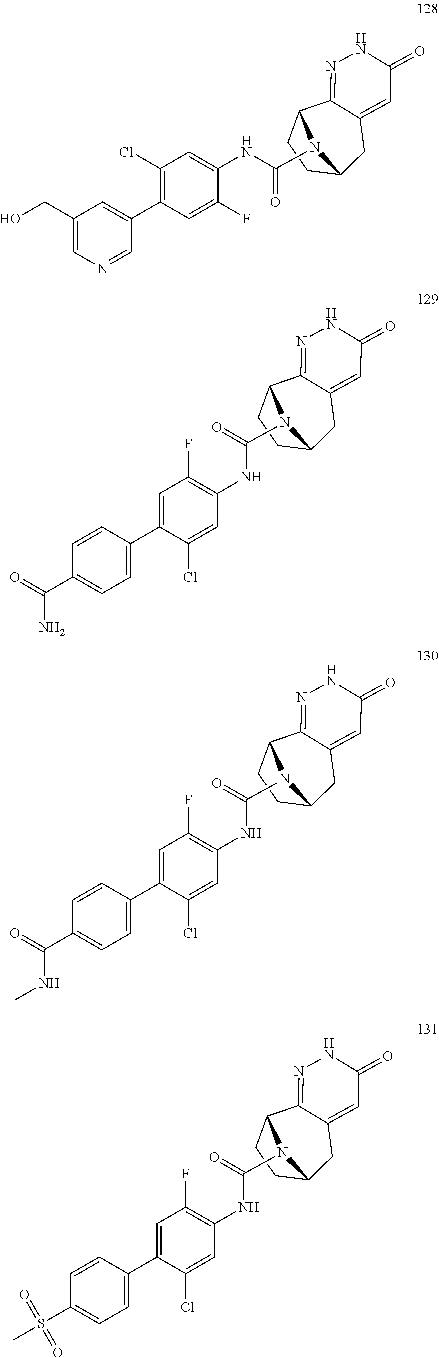 [SMILES: O=C(Nc1cc(Cl)c(-c2cncc(CO)c2)cc1F)N1[C@H]2CC[C@@H]1c1n[nH]c(=O)cc1C2 ; NC(=O)c1ccc(-c2cc(F)c(NC(=O)N3[C@H]4CC[C@@H]3c3n[nH]c(=O)cc3C4)cc2Cl)cc1 ; CNC(=O)c1ccc(-c2cc(F)c(NC(=O)N3[C@H]4CC[C@@H]3c3n[nH]c(=O)cc3C4)cc2Cl)cc1 ; CS(=O)(=O)c1ccc(-c2cc(F)c(NC(=O)N3[C@H]4CC[C@@H]3c3n[nH]c(=O)cc3C4)cc2Cl)cc1]

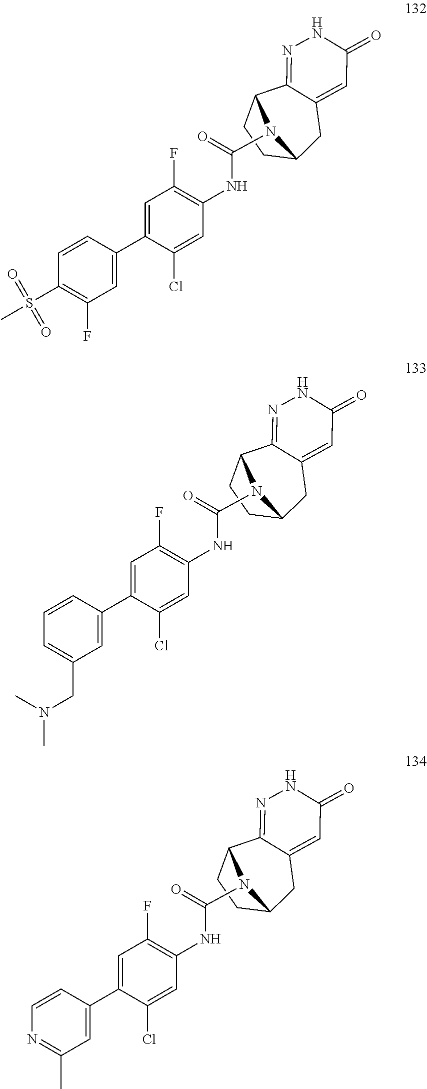 [SMILES: CS(=O)(=O)c1ccc(-c2cc(F)c(NC(=O)N3[C@H]4CC[C@@H]3c3n[nH]c(=O)cc3C4)cc2Cl)cc1F ; CN(C)Cc1cccc(-c2cc(F)c(NC(=O)N3[C@H]4CC[C@@H]3c3n[nH]c(=O)cc3C4)cc2Cl)c1 ; Cc1cc(-c2cc(F)c(NC(=O)N3[C@H]4CC[C@@H]3c3n[nH]c(=O)cc3C4)cc2Cl)ccn1]

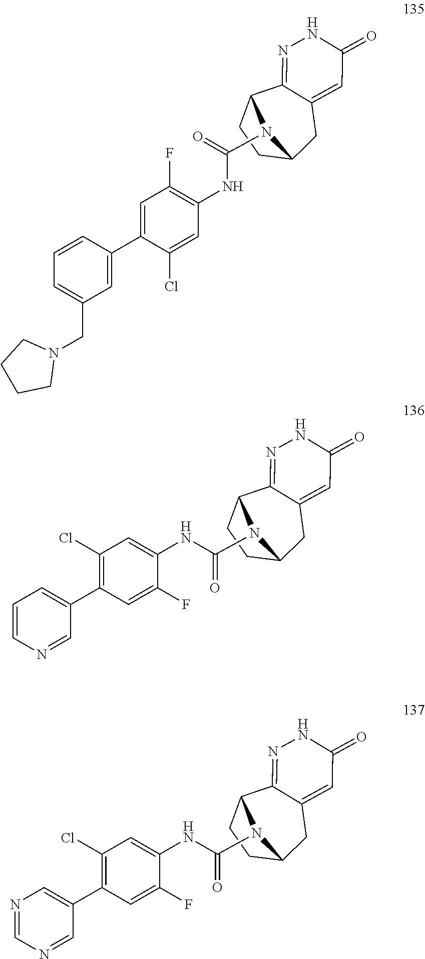 [SMILES: O=C(Nc1cc(Cl)c(-c2cccc(CN3CCCC3)c2)cc1F)N1[C@H]2CC[C@@H]1c1n[nH]c(=O)cc1C2 ; O=C(Nc1cc(Cl)c(-c2cccnc2)cc1F)N1[C@H]2CC[C@@H]1c1n[nH]c(=O)cc1C2 ; O=C(Nc1cc(Cl)c(-c2cncnc2)cc1F)N1[C@H]2CC[C@@H]1c1n[nH]c(=O)cc1C2]

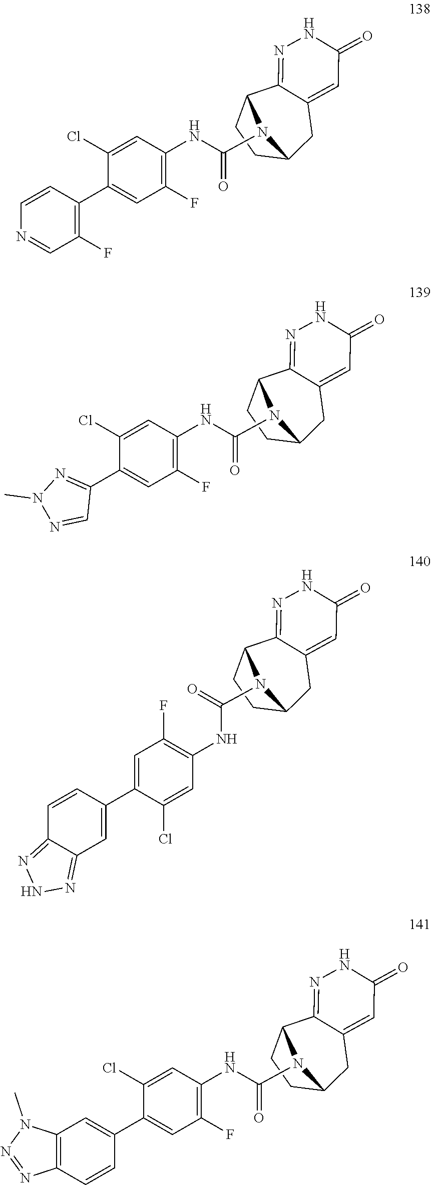 [SMILES: O=C(Nc1cc(Cl)c(-c2ccncc2F)cc1F)N1[C@H]2CC[C@@H]1c1n[nH]c(=O)cc1C2 ; Cn1ncc(-c2cc(F)c(NC(=O)N3[C@H]4CC[C@@H]3c3n[nH]c(=O)cc3C4)cc2Cl)n1 ; O=C(Nc1cc(Cl)c(-c2ccc3n[nH]nc3c2)cc1F)N1[C@H]2CC[C@@H]1c1n[nH]c(=O)cc1C2 ; Cn1nnc2ccc(-c3cc(F)c(NC(=O)N4[C@H]5CC[C@@H]4c4n[nH]c(=O)cc4C5)cc3Cl)cc21]

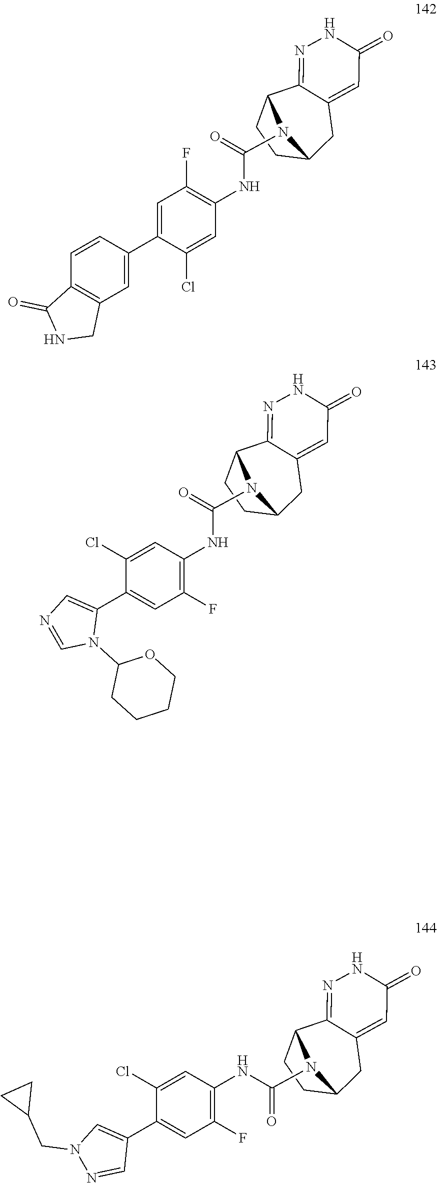 [SMILES: O=C1NCc2cc(-c3cc(F)c(NC(=O)N4[C@H]5CC[C@@H]4c4n[nH]c(=O)cc4C5)cc3Cl)ccc21 ; O=C(Nc1cc(Cl)c(-c2cncn2C2CCCCO2)cc1F)N1[C@H]2CC[C@@H]1c1n[nH]c(=O)cc1C2 ; O=C(Nc1cc(Cl)c(-c2cnn(CC3CC3)c2)cc1F)N1[C@H]2CC[C@@H]1c1n[nH]c(=O)cc1C2]

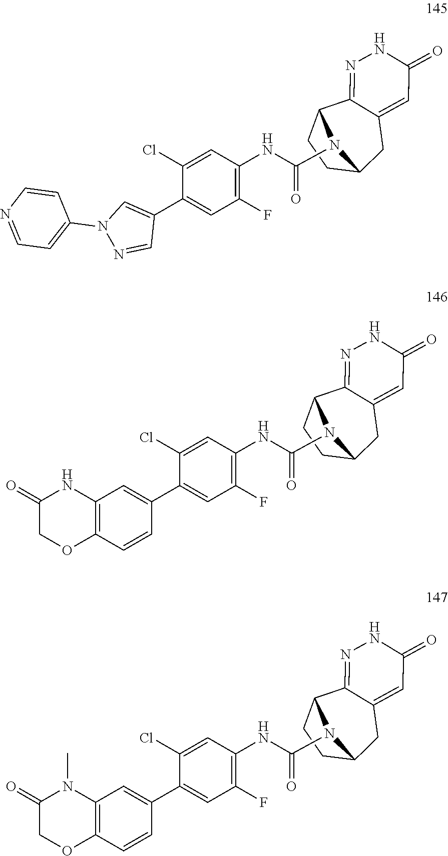 [SMILES: O=C(Nc1cc(Cl)c(-c2cnn(-c3ccncc3)c2)cc1F)N1[C@H]2CC[C@@H]1c1n[nH]c(=O)cc1C2 ; O=C1COc2ccc(-c3cc(F)c(NC(=O)N4[C@H]5CC[C@@H]4c4n[nH]c(=O)cc4C5)cc3Cl)cc2N1 ; CN1C(=O)COc2ccc(-c3cc(F)c(NC(=O)N4[C@H]5CC[C@@H]4c4n[nH]c(=O)cc4C5)cc3Cl)cc21]

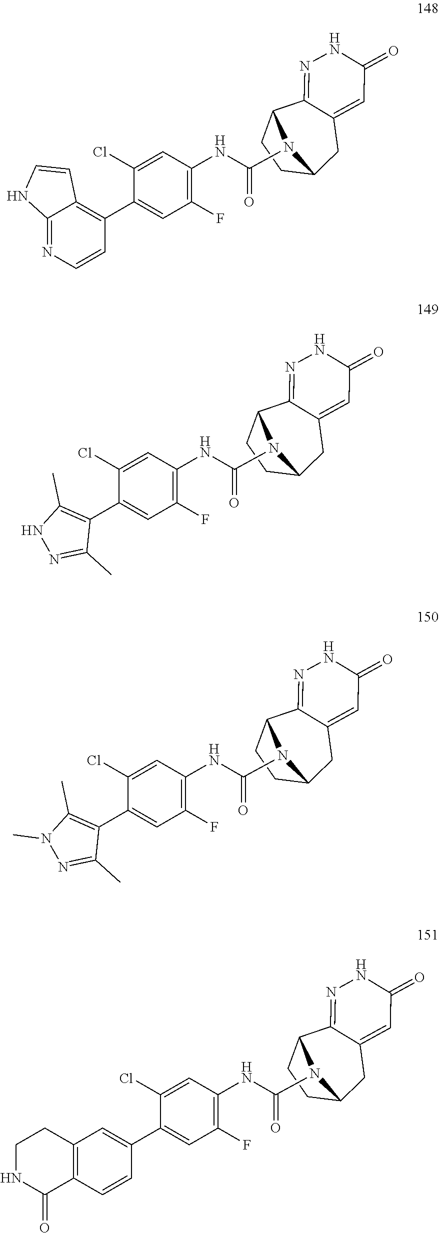 [SMILES: O=C(Nc1cc(Cl)c(-c2ccnc3[nH]ccc23)cc1F)N1[C@H]2CC[C@@H]1c1n[nH]c(=O)cc1C2 ; Cc1n[nH]c(C)c1-c1cc(F)c(NC(=O)N2[C@H]3CC[C@@H]2c2n[nH]c(=O)cc2C3)cc1Cl ; Cc1nn(C)c(C)c1-c1cc(F)c(NC(=O)N2[C@H]3CC[C@@H]2c2n[nH]c(=O)cc2C3)cc1Cl ; O=C1NCCc2cc(-c3cc(F)c(NC(=O)N4[C@H]5CC[C@@H]4c4n[nH]c(=O)cc4C5)cc3Cl)ccc21]

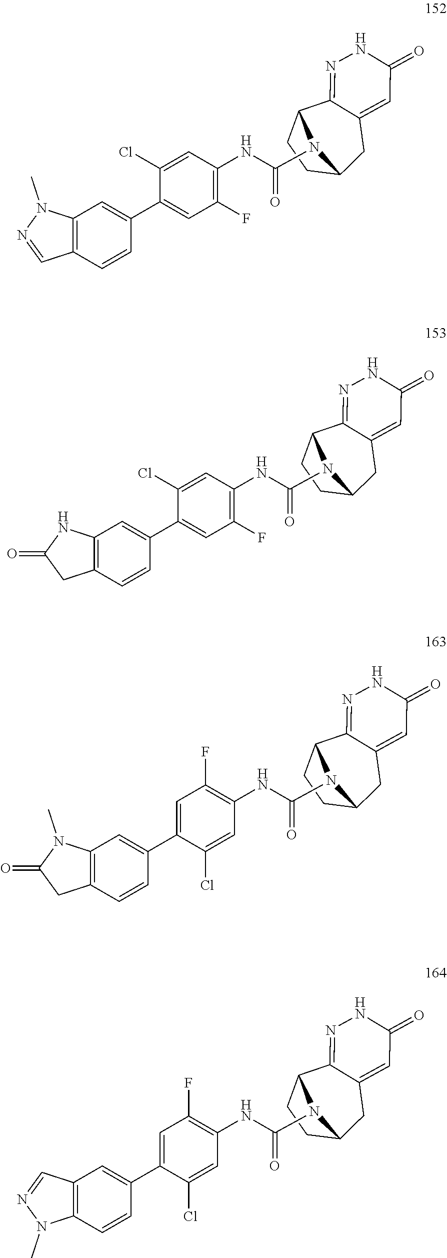 [SMILES: Cn1ncc2ccc(-c3cc(F)c(NC(=O)N4[C@H]5CC[C@@H]4c4n[nH]c(=O)cc4C5)cc3Cl)cc21 ; O=C1Cc2ccc(-c3cc(F)c(NC(=O)N4[C@H]5CC[C@@H]4c4n[nH]c(=O)cc4C5)cc3Cl)cc2N1 ; CN1C(=O)Cc2ccc(-c3cc(F)c(NC(=O)N4[C@H]5CC[C@@H]4c4n[nH]c(=O)cc4C5)cc3Cl)cc21 ; Cn1ncc2cc(-c3cc(F)c(NC(=O)N4[C@H]5CC[C@@H]4c4n[nH]c(=O)cc4C5)cc3Cl)ccc21]

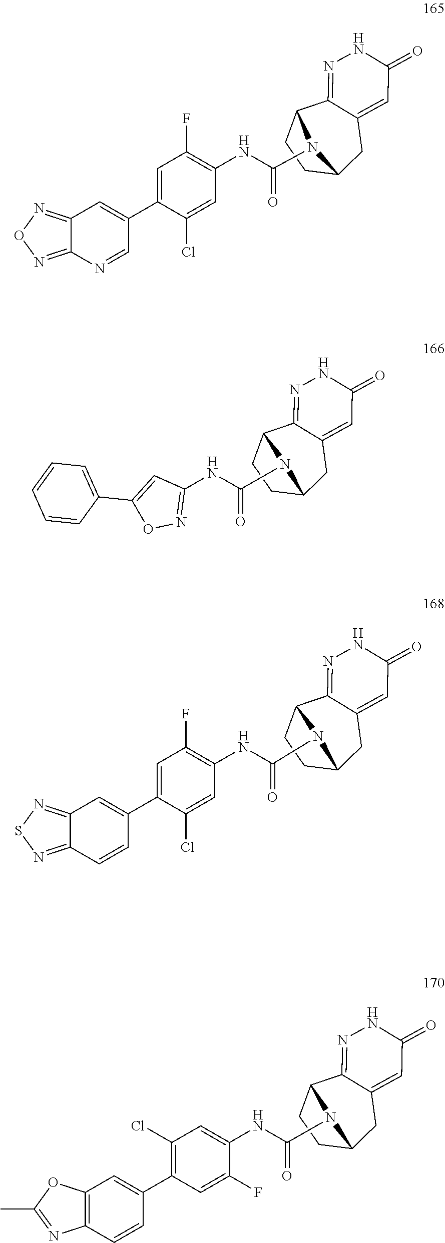 [SMILES: O=C(Nc1cc(Cl)c(-c2cnc3nonc3c2)cc1F)N1[C@H]2CC[C@@H]1c1n[nH]c(=O)cc1C2 ; O=C(Nc1cc(-c2ccccc2)on1)N1[C@H]2CC[C@@H]1c1n[nH]c(=O)cc1C2 ; O=C(Nc1cc(Cl)c(-c2ccc3nsnc3c2)cc1F)N1[C@H]2CC[C@@H]1c1n[nH]c(=O)cc1C2 ; Cc1nc2ccc(-c3cc(F)c(NC(=O)N4[C@H]5CC[C@@H]4c4n[nH]c(=O)cc4C5)cc3Cl)cc2o1]

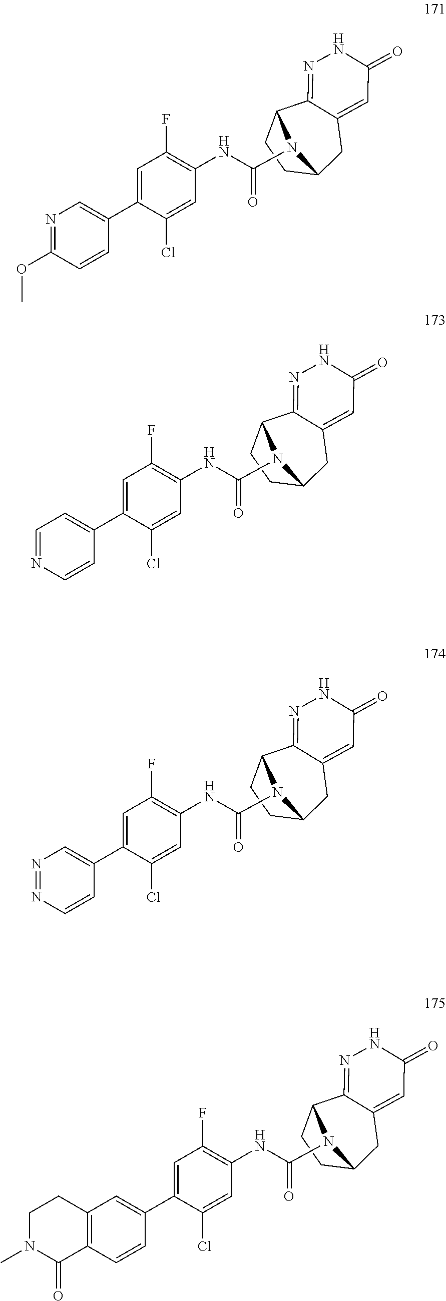 [SMILES: COc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccncc2)cc1F)N1[C@H]2CC[C@@H]1c1n[nH]c(=O)cc1C2 ; O=C(Nc1cc(Cl)c(-c2ccnnc2)cc1F)N1[C@H]2CC[C@@H]1c1n[nH]c(=O)cc1C2 ; CN1CCc2cc(-c3cc(F)c(NC(=O)N4[C@H]5CC[C@@H]4c4n[nH]c(=O)cc4C5)cc3Cl)ccc2C1=O]

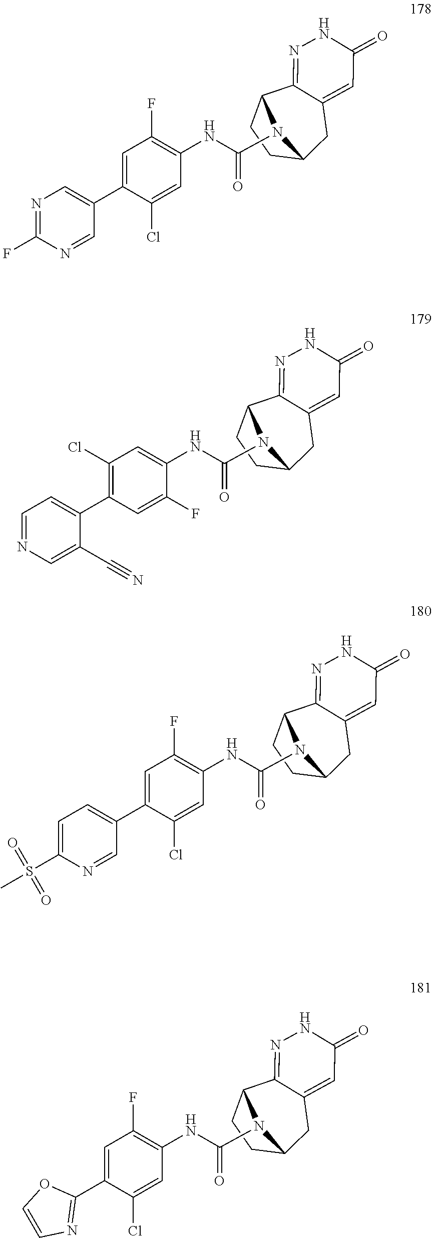 [SMILES: O=C(Nc1cc(Cl)c(-c2cnc(F)nc2)cc1F)N1[C@H]2CC[C@@H]1c1n[nH]c(=O)cc1C2 ; N#Cc1cnccc1-c1cc(F)c(NC(=O)N2[C@H]3CC[C@@H]2c2n[nH]c(=O)cc2C3)cc1Cl ; CS(=O)(=O)c1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ncco2)cc1F)N1[C@H]2CC[C@@H]1c1n[nH]c(=O)cc1C2]

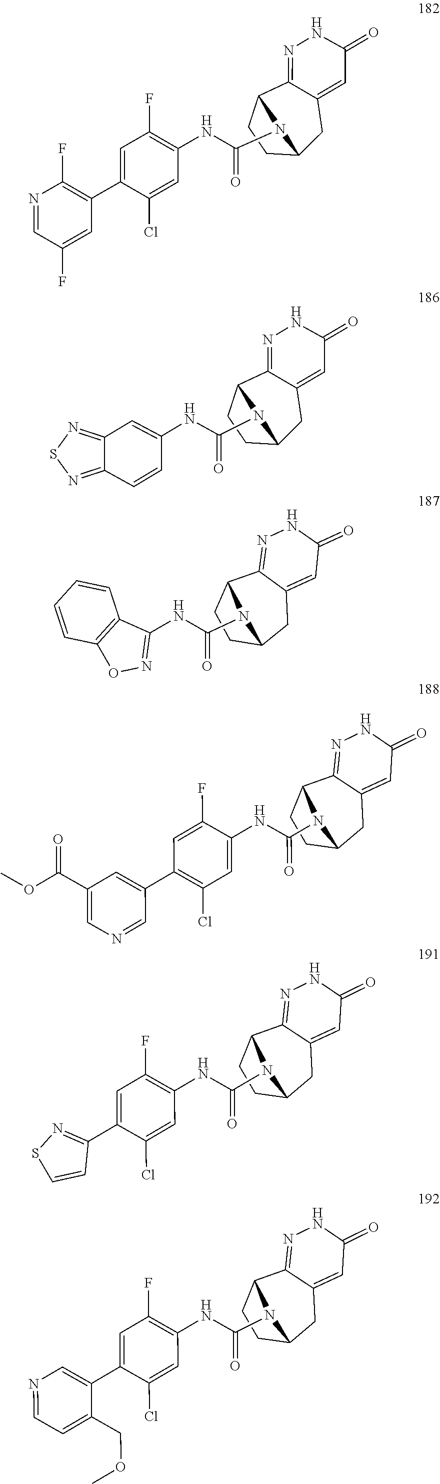 [SMILES: O=C(Nc1cc(Cl)c(-c2cc(F)cnc2F)cc1F)N1[C@H]2CC[C@@H]1c1n[nH]c(=O)cc1C2 ; O=C(Nc1ccc2nsnc2c1)N1[C@H]2CC[C@@H]1c1n[nH]c(=O)cc1C2 ; O=C(Nc1noc2ccccc12)N1[C@H]2CC[C@@H]1c1n[nH]c(=O)cc1C2 ; COC(=O)c1cncc(-c2cc(F)c(NC(=O)N3[C@H]4CC[C@@H]3c3n[nH]c(=O)cc3C4)cc2Cl)c1 ; O=C(Nc1cc(Cl)c(-c2ccsn2)cc1F)N1[C@H]2CC[C@@H]1c1n[nH]c(=O)cc1C2 ; COCc1ccncc1-c1cc(F)c(NC(=O)N2[C@H]3CC[C@@H]2c2n[nH]c(=O)cc2C3)cc1Cl]

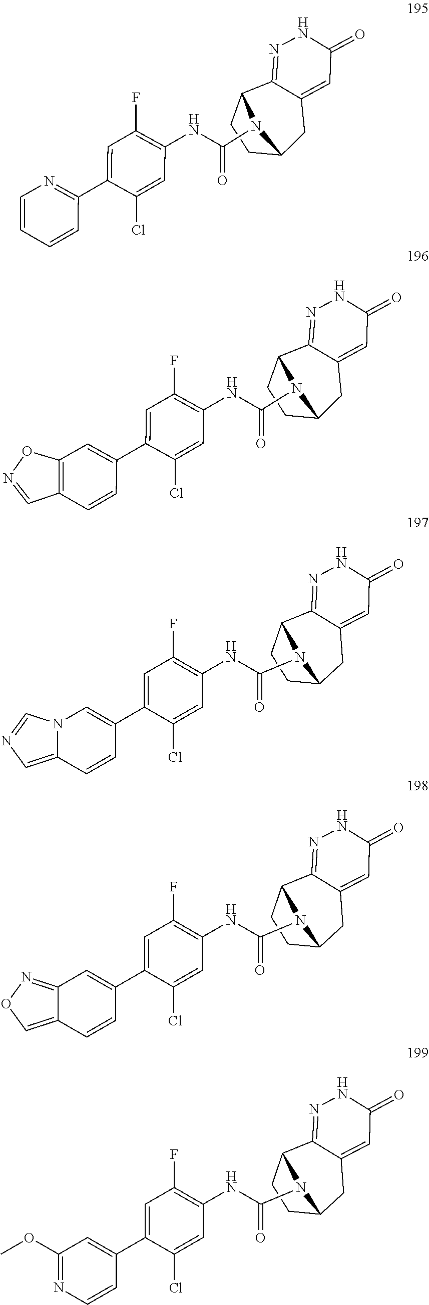 [SMILES: O=C(Nc1cc(Cl)c(-c2ccccn2)cc1F)N1[C@H]2CC[C@@H]1c1n[nH]c(=O)cc1C2 ; O=C(Nc1cc(Cl)c(-c2ccc3cnoc3c2)cc1F)N1[C@H]2CC[C@@H]1c1n[nH]c(=O)cc1C2 ; O=C(Nc1cc(Cl)c(-c2ccc3cncn3c2)cc1F)N1[C@H]2CC[C@@H]1c1n[nH]c(=O)cc1C2 ; O=C(Nc1cc(Cl)c(-c2ccc3conc3c2)cc1F)N1[C@H]2CC[C@@H]1c1n[nH]c(=O)cc1C2 ; COc1cc(-c2cc(F)c(NC(=O)N3[C@H]4CC[C@@H]3c3n[nH]c(=O)cc3C4)cc2Cl)ccn1]

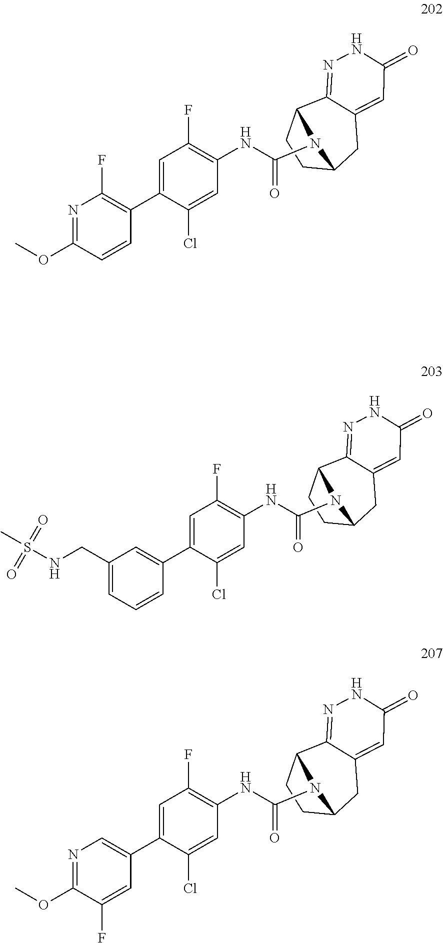 [SMILES: COc1ccc(-c2cc(F)c(NC(=O)N3[C@H]4CC[C@@H]3c3n[nH]c(=O)cc3C4)cc2Cl)c(F)n1 ; CS(=O)(=O)NCc1cccc(-c2cc(F)c(NC(=O)N3[C@H]4CC[C@@H]3c3n[nH]c(=O)cc3C4)cc2Cl)c1 ; COc1ncc(-c2cc(F)c(NC(=O)N3[C@H]4CC[C@@H]3c3n[nH]c(=O)cc3C4)cc2Cl)cc1F]

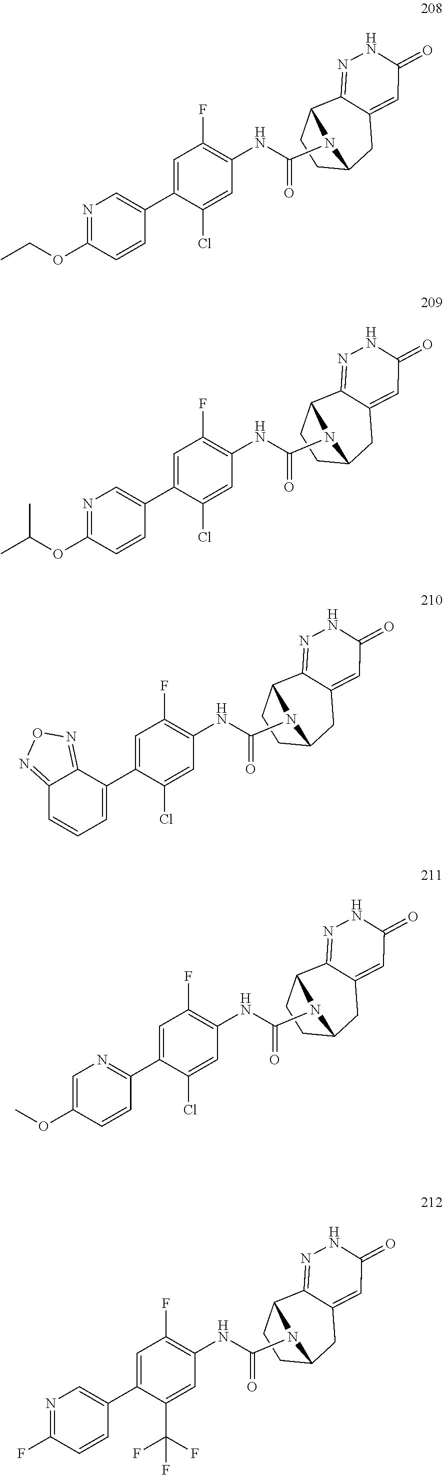 [SMILES: CCOc1ccc(-c2cc(F)c(NC(=O)N3[C@H]4CC[C@@H]3c3n[nH]c(=O)cc3C4)cc2Cl)cn1 ; CC(C)Oc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2cccc3nonc23)cc1F)N1[C@H]2CC[C@@H]1c1n[nH]c(=O)cc1C2 ; COc1ccc(-c2cc(F)c(NC(=O)N3[C@H]4CC[C@@H]3c3n[nH]c(=O)cc3C4)cc2Cl)nc1 ; O=C(Nc1cc(C(F)(F)F)c(-c2ccc(F)nc2)cc1F)N1[C@H]2CC[C@@H]1c1n[nH]c(=O)cc1C2]

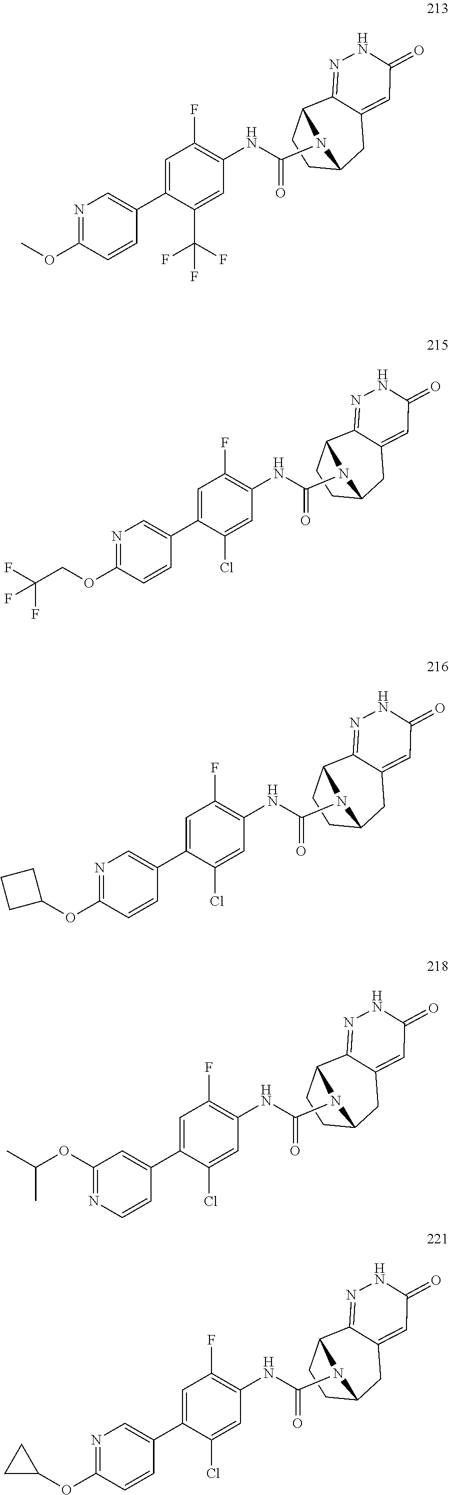 [SMILES: COc1ccc(-c2cc(F)c(NC(=O)N3[C@H]4CC[C@@H]3c3n[nH]c(=O)cc3C4)cc2C(F)(F)F)cn1 ; O=C(Nc1cc(Cl)c(-c2ccc(OCC(F)(F)F)nc2)cc1F)N1[C@H]2CC[C@@H]1c1n[nH]c(=O)cc1C2 ; O=C(Nc1cc(Cl)c(-c2ccc(OC3CCC3)nc2)cc1F)N1[C@H]2CC[C@@H]1c1n[nH]c(=O)cc1C2 ; CC(C)Oc1cc(-c2cc(F)c(NC(=O)N3[C@H]4CC[C@@H]3c3n[nH]c(=O)cc3C4)cc2Cl)ccn1 ; O=C(Nc1cc(Cl)c(-c2ccc(OC3CC3)nc2)cc1F)N1[C@H]2CC[C@@H]1c1n[nH]c(=O)cc1C2]

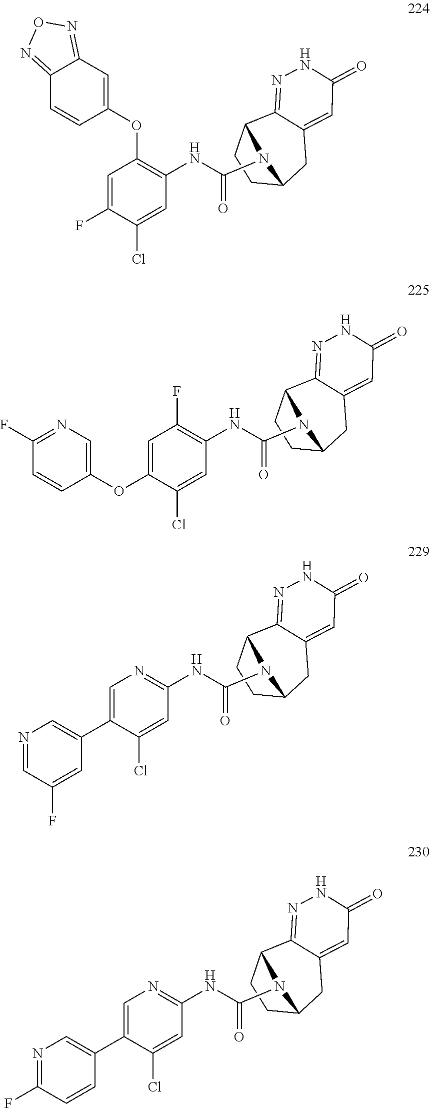 [SMILES: O=C(Nc1cc(Cl)c(F)cc1Oc1ccc2nonc2c1)N1[C@H]2CC[C@@H]1c1n[nH]c(=O)cc1C2 ; O=C(Nc1cc(Cl)c(Oc2ccc(F)nc2)cc1F)N1[C@H]2CC[C@@H]1c1n[nH]c(=O)cc1C2 ; O=C(Nc1cc(Cl)c(-c2cncc(F)c2)cn1)N1[C@H]2CC[C@@H]1c1n[nH]c(=O)cc1C2 ; O=C(Nc1cc(Cl)c(-c2ccc(F)nc2)cn1)N1[C@H]2CC[C@@H]1c1n[nH]c(=O)cc1C2]

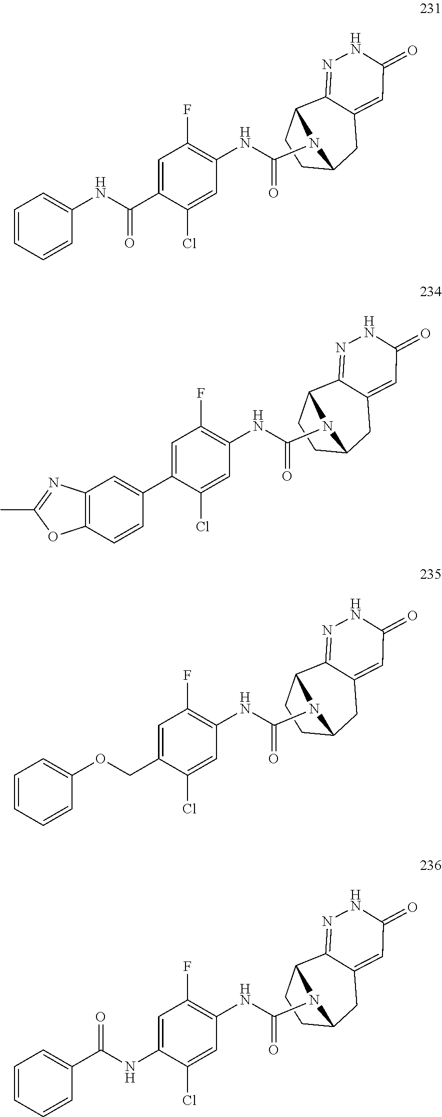 [SMILES: O=C(Nc1ccccc1)c1cc(F)c(NC(=O)N2[C@H]3CC[C@@H]2c2n[nH]c(=O)cc2C3)cc1Cl ; Cc1nc2cc(-c3cc(F)c(NC(=O)N4[C@H]5CC[C@@H]4c4n[nH]c(=O)cc4C5)cc3Cl)ccc2o1 ; O=C(Nc1cc(Cl)c(COc2ccccc2)cc1F)N1[C@H]2CC[C@@H]1c1n[nH]c(=O)cc1C2 ; O=C(Nc1cc(F)c(NC(=O)N2[C@H]3CC[C@@H]2c2n[nH]c(=O)cc2C3)cc1Cl)c1ccccc1]

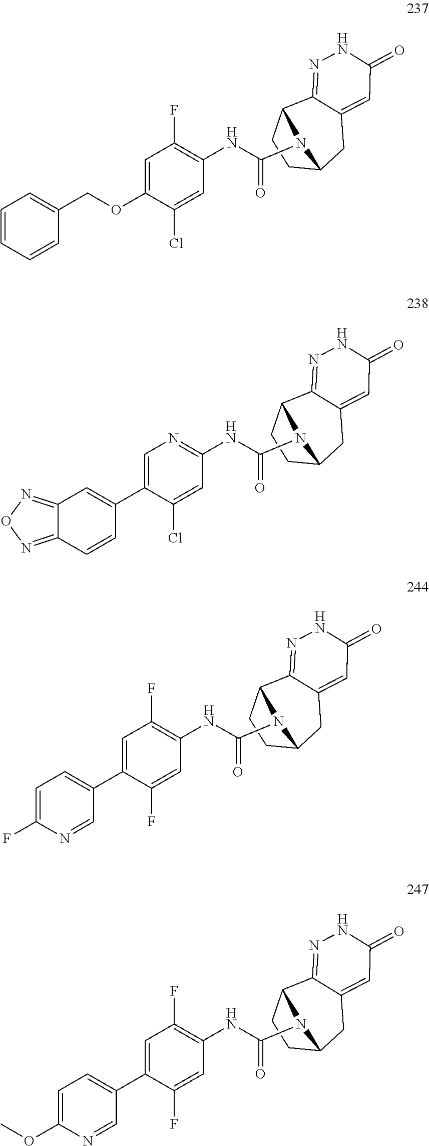 [SMILES: O=C(Nc1cc(Cl)c(OCc2ccccc2)cc1F)N1[C@H]2CC[C@@H]1c1n[nH]c(=O)cc1C2 ; O=C(Nc1cc(Cl)c(-c2ccc3nonc3c2)cn1)N1[C@H]2CC[C@@H]1c1n[nH]c(=O)cc1C2 ; O=C(Nc1cc(F)c(-c2ccc(F)nc2)cc1F)N1[C@H]2CC[C@@H]1c1n[nH]c(=O)cc1C2 ; COc1ccc(-c2cc(F)c(NC(=O)N3[C@H]4CC[C@@H]3c3n[nH]c(=O)cc3C4)cc2F)cn1]

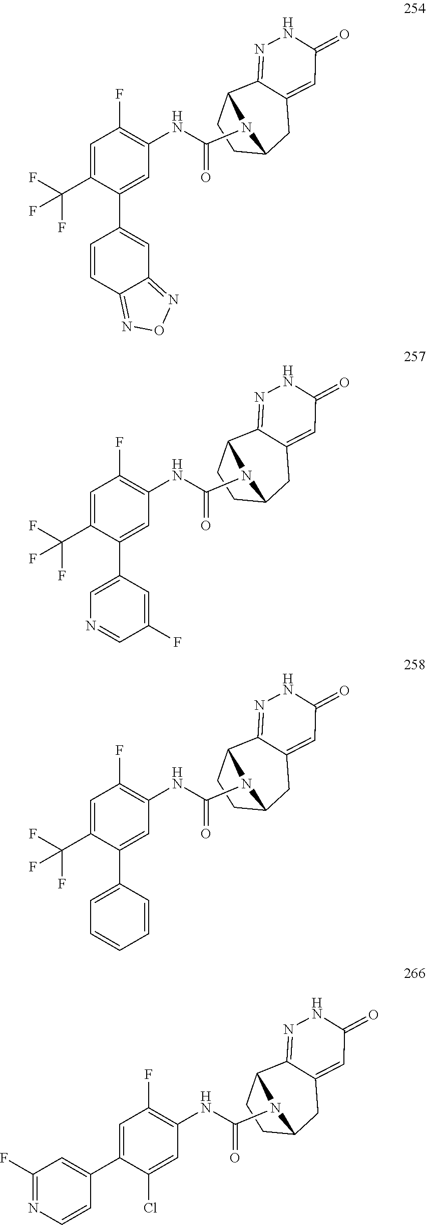 [SMILES: O=C(Nc1cc(-c2ccc3nonc3c2)c(C(F)(F)F)cc1F)N1[C@H]2CC[C@@H]1c1n[nH]c(=O)cc1C2 ; O=C(Nc1cc(-c2cncc(F)c2)c(C(F)(F)F)cc1F)N1[C@H]2CC[C@@H]1c1n[nH]c(=O)cc1C2 ; O=C(Nc1cc(-c2ccccc2)c(C(F)(F)F)cc1F)N1[C@H]2CC[C@@H]1c1n[nH]c(=O)cc1C2 ; O=C(Nc1cc(Cl)c(-c2ccnc(F)c2)cc1F)N1[C@H]2CC[C@@H]1c1n[nH]c(=O)cc1C2]

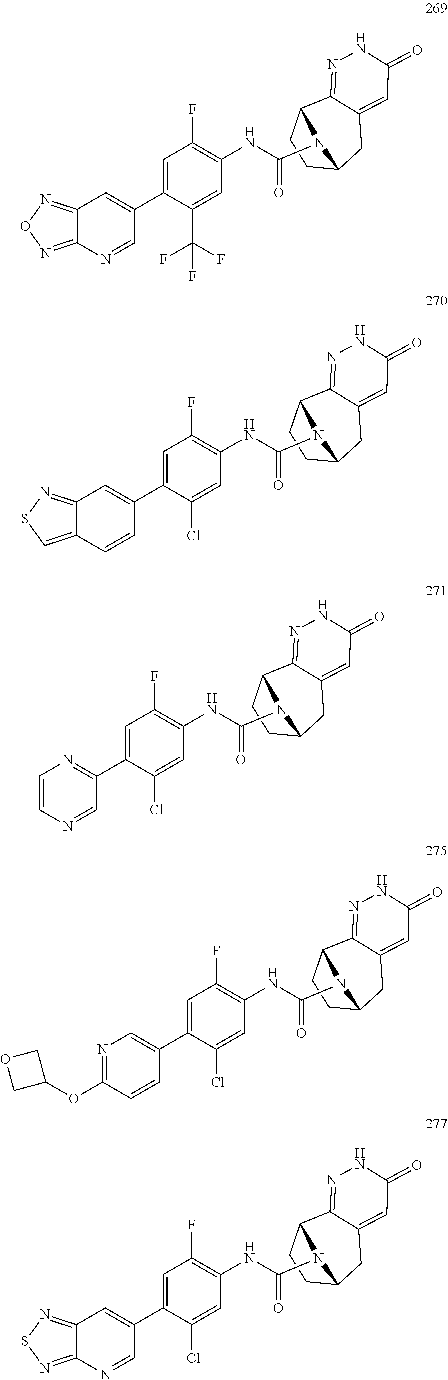 [SMILES: O=C(Nc1cc(C(F)(F)F)c(-c2cnc3nonc3c2)cc1F)N1[C@H]2CC[C@@H]1c1n[nH]c(=O)cc1C2 ; O=C(Nc1cc(Cl)c(-c2ccc3csnc3c2)cc1F)N1[C@H]2CC[C@@H]1c1n[nH]c(=O)cc1C2 ; O=C(Nc1cc(Cl)c(-c2cnccn2)cc1F)N1[C@H]2CC[C@@H]1c1n[nH]c(=O)cc1C2 ; O=C(Nc1cc(Cl)c(-c2ccc(OC3COC3)nc2)cc1F)N1[C@H]2CC[C@@H]1c1n[nH]c(=O)cc1C2 ; O=C(Nc1cc(Cl)c(-c2cnc3nsnc3c2)cc1F)N1[C@H]2CC[C@@H]1c1n[nH]c(=O)cc1C2]

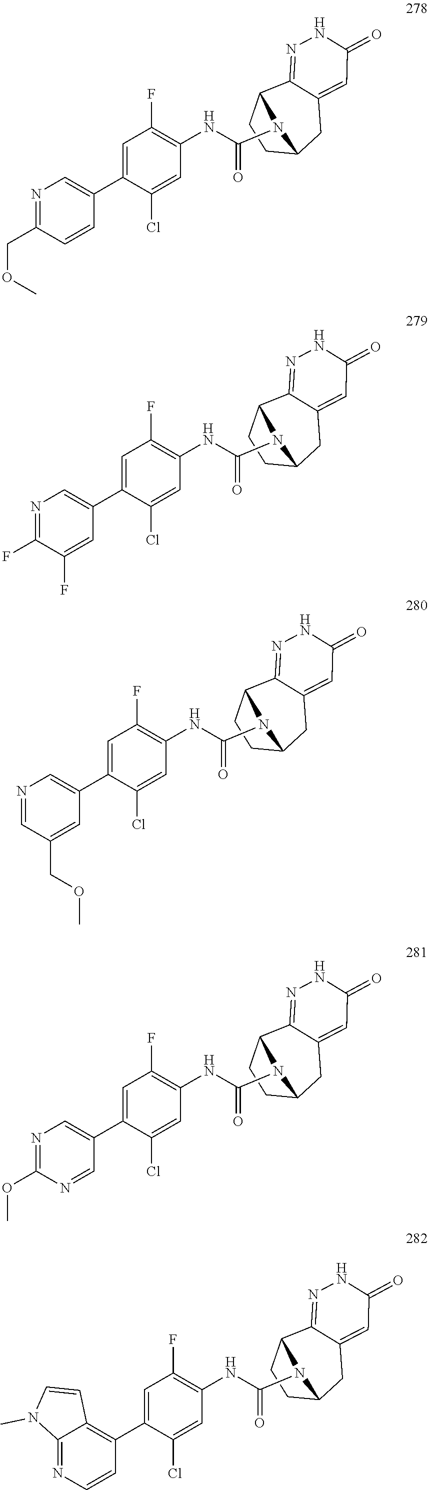 [SMILES: COCc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2cnc(F)c(F)c2)cc1F)N1[C@H]2CC[C@@H]1c1n[nH]c(=O)cc1C2 ; COCc1cncc(-c2cc(F)c(NC(=O)N3[C@H]4CC[C@@H]3c3n[nH]c(=O)cc3C4)cc2Cl)c1 ; COc1ncc(-c2cc(F)c(NC(=O)N3[C@H]4CC[C@@H]3c3n[nH]c(=O)cc3C4)cc2Cl)cn1 ; Cn1ccc2c(-c3cc(F)c(NC(=O)N4[C@H]5CC[C@@H]4c4n[nH]c(=O)cc4C5)cc3Cl)ccnc21]

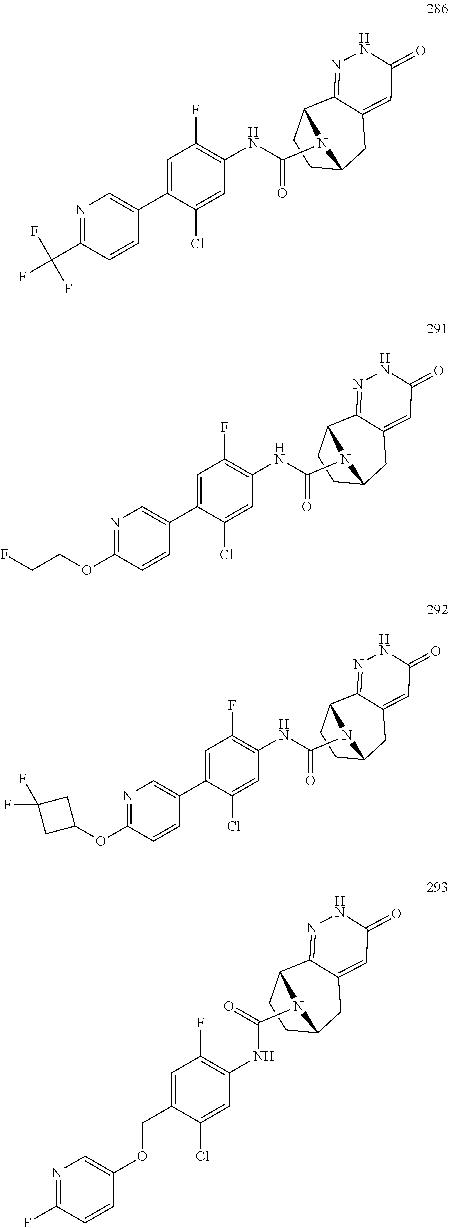 [SMILES: O=C(Nc1cc(Cl)c(-c2ccc(C(F)(F)F)nc2)cc1F)N1[C@H]2CC[C@@H]1c1n[nH]c(=O)cc1C2 ; O=C(Nc1cc(Cl)c(-c2ccc(OCCF)nc2)cc1F)N1[C@H]2CC[C@@H]1c1n[nH]c(=O)cc1C2 ; O=C(Nc1cc(Cl)c(-c2ccc(OC3CC(F)(F)C3)nc2)cc1F)N1[C@H]2CC[C@@H]1c1n[nH]c(=O)cc1C2 ; O=C(Nc1cc(Cl)c(COc2ccc(F)nc2)cc1F)N1[C@H]2CC[C@@H]1c1n[nH]c(=O)cc1C2]

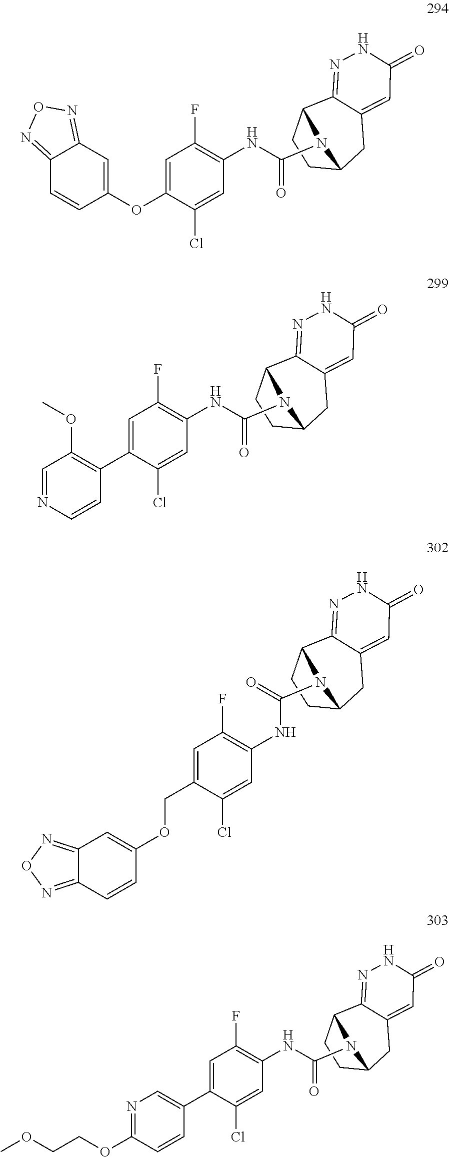 [SMILES: O=C(Nc1cc(Cl)c(Oc2ccc3nonc3c2)cc1F)N1[C@H]2CC[C@@H]1c1n[nH]c(=O)cc1C2 ; COc1cnccc1-c1cc(F)c(NC(=O)N2[C@H]3CC[C@@H]2c2n[nH]c(=O)cc2C3)cc1Cl ; O=C(Nc1cc(Cl)c(COc2ccc3nonc3c2)cc1F)N1[C@H]2CC[C@@H]1c1n[nH]c(=O)cc1C2 ; COCCOc1ccc(-c2cc(F)c(NC(=O)N3[C@H]4CC[C@@H]3c3n[nH]c(=O)cc3C4)cc2Cl)cn1]

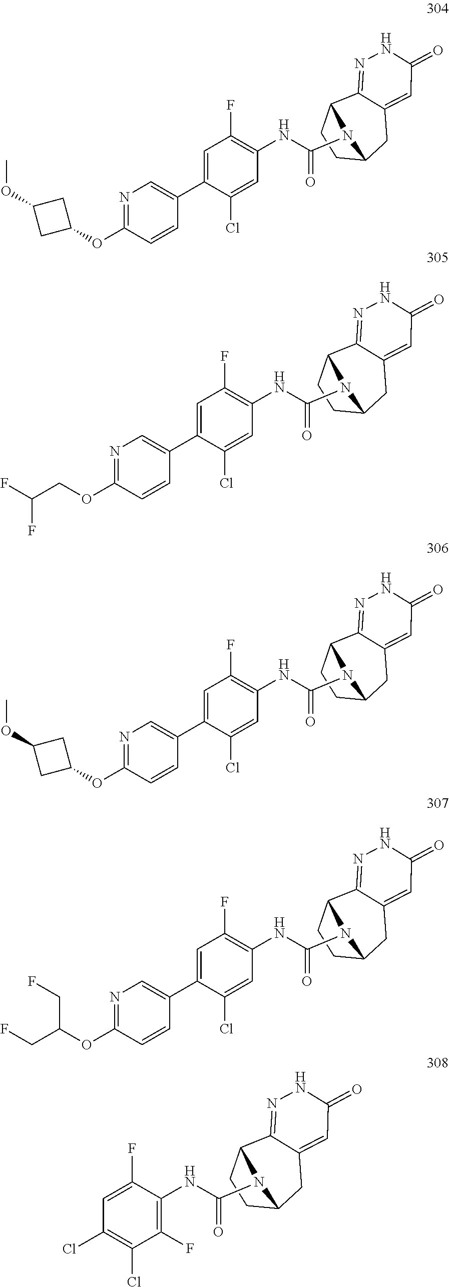 [SMILES: CO[C@H]1C[C@@H](Oc2ccc(-c3cc(F)c(NC(=O)N4[C@H]5CC[C@@H]4c4n[nH]c(=O)cc4C5)cc3Cl)cn2)C1 ; O=C(Nc1cc(Cl)c(-c2ccc(OCC(F)F)nc2)cc1F)N1[C@H]2CC[C@@H]1c1n[nH]c(=O)cc1C2 ; CO[C@H]1C[C@H](Oc2ccc(-c3cc(F)c(NC(=O)N4[C@H]5CC[C@@H]4c4n[nH]c(=O)cc4C5)cc3Cl)cn2)C1 ; O=C(Nc1cc(Cl)c(-c2ccc(OC(CF)CF)nc2)cc1F)N1[C@H]2CC[C@@H]1c1n[nH]c(=O)cc1C2 ; O=C(Nc1c(F)cc(Cl)c(Cl)c1F)N1[C@H]2CC[C@@H]1c1n[nH]c(=O)cc1C2]

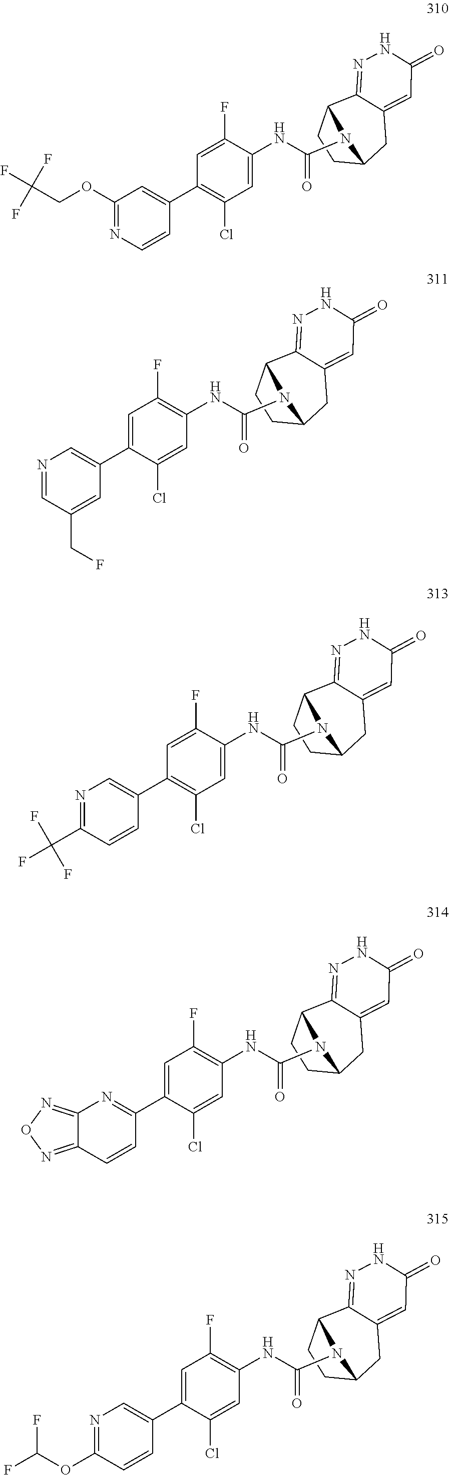 [SMILES: O=C(Nc1cc(Cl)c(-c2ccnc(OCC(F)(F)F)c2)cc1F)N1[C@H]2CC[C@@H]1c1n[nH]c(=O)cc1C2 ; O=C(Nc1cc(Cl)c(-c2cncc(CF)c2)cc1F)N1[C@H]2CC[C@@H]1c1n[nH]c(=O)cc1C2 ; O=C(Nc1cc(Cl)c(-c2ccc(C(F)(F)F)nc2)cc1F)N1[C@H]2CC[C@@H]1c1n[nH]c(=O)cc1C2 ; O=C(Nc1cc(Cl)c(-c2ccc3nonc3n2)cc1F)N1[C@H]2CC[C@@H]1c1n[nH]c(=O)cc1C2 ; O=C(Nc1cc(Cl)c(-c2ccc(OC(F)F)nc2)cc1F)N1[C@H]2CC[C@@H]1c1n[nH]c(=O)cc1C2]

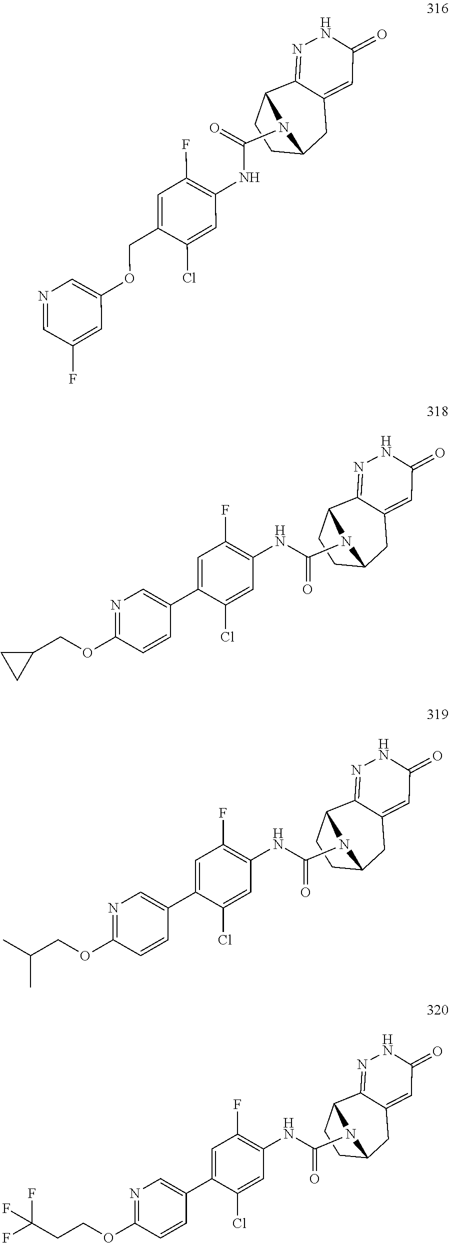 [SMILES: O=C(Nc1cc(Cl)c(COc2cncc(F)c2)cc1F)N1[C@H]2CC[C@@H]1c1n[nH]c(=O)cc1C2 ; O=C(Nc1cc(Cl)c(-c2ccc(OCC3CC3)nc2)cc1F)N1[C@H]2CC[C@@H]1c1n[nH]c(=O)cc1C2 ; CC(C)COc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccc(OCCC(F)(F)F)nc2)cc1F)N1[C@H]2CC[C@@H]1c1n[nH]c(=O)cc1C2]

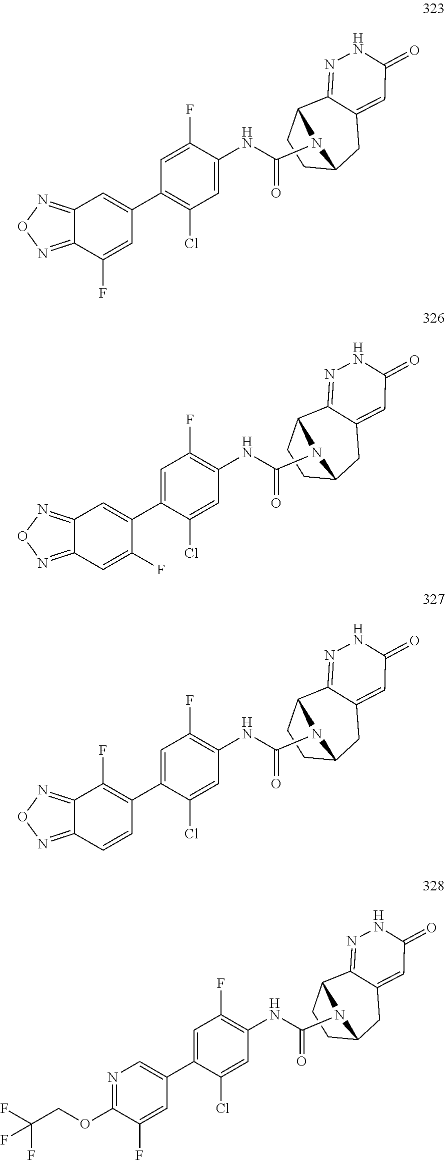 [SMILES: O=C(Nc1cc(Cl)c(-c2cc(F)c3nonc3c2)cc1F)N1[C@H]2CC[C@@H]1c1n[nH]c(=O)cc1C2 ; O=C(Nc1cc(Cl)c(-c2cc3nonc3cc2F)cc1F)N1[C@H]2CC[C@@H]1c1n[nH]c(=O)cc1C2 ; O=C(Nc1cc(Cl)c(-c2ccc3nonc3c2F)cc1F)N1[C@H]2CC[C@@H]1c1n[nH]c(=O)cc1C2 ; O=C(Nc1cc(Cl)c(-c2cnc(OCC(F)(F)F)c(F)c2)cc1F)N1[C@H]2CC[C@@H]1c1n[nH]c(=O)cc1C2]

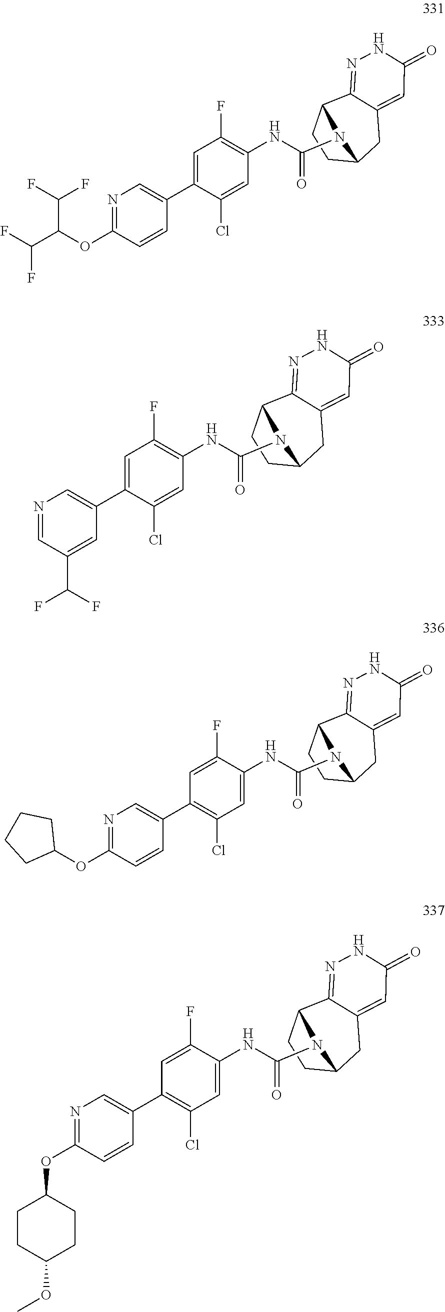 [SMILES: O=C(Nc1cc(Cl)c(-c2ccc(OC(C(F)F)C(F)F)nc2)cc1F)N1[C@H]2CC[C@@H]1c1n[nH]c(=O)cc1C2 ; O=C(Nc1cc(Cl)c(-c2cncc(C(F)F)c2)cc1F)N1[C@H]2CC[C@@H]1c1n[nH]c(=O)cc1C2 ; O=C(Nc1cc(Cl)c(-c2ccc(OC3CCCC3)nc2)cc1F)N1[C@H]2CC[C@@H]1c1n[nH]c(=O)cc1C2 ; CO[C@H]1CC[C@H](Oc2ccc(-c3cc(F)c(NC(=O)N4[C@H]5CC[C@@H]4c4n[nH]c(=O)cc4C5)cc3Cl)cn2)CC1]

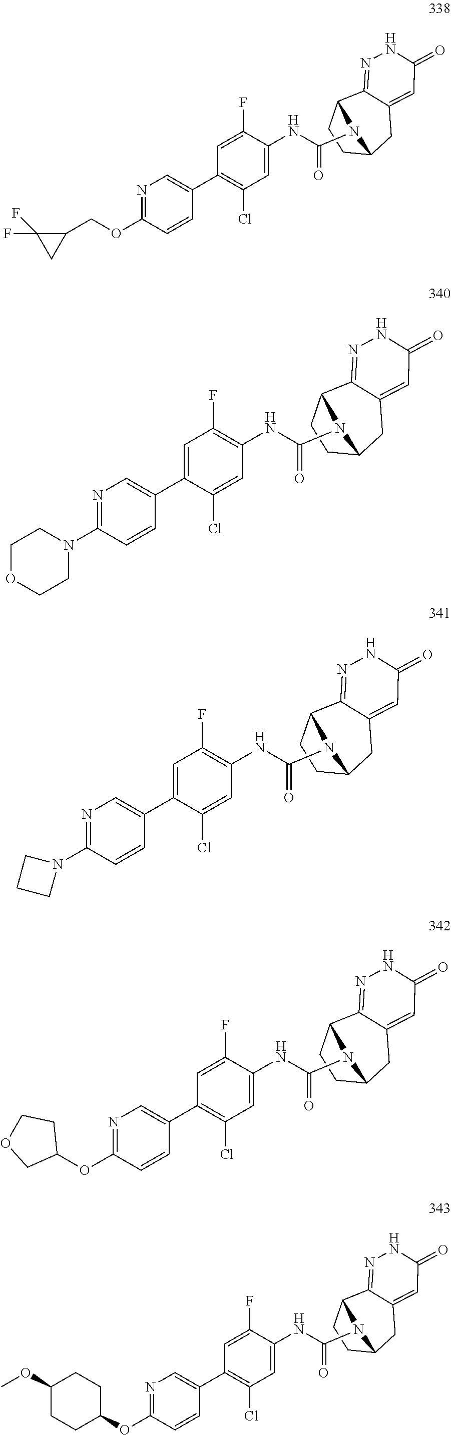 [SMILES: O=C(Nc1cc(Cl)c(-c2ccc(OCC3CC3(F)F)nc2)cc1F)N1[C@H]2CC[C@@H]1c1n[nH]c(=O)cc1C2 ; O=C(Nc1cc(Cl)c(-c2ccc(N3CCOCC3)nc2)cc1F)N1[C@H]2CC[C@@H]1c1n[nH]c(=O)cc1C2 ; O=C(Nc1cc(Cl)c(-c2ccc(N3CCC3)nc2)cc1F)N1[C@H]2CC[C@@H]1c1n[nH]c(=O)cc1C2 ; O=C(Nc1cc(Cl)c(-c2ccc(OC3CCOC3)nc2)cc1F)N1[C@H]2CC[C@@H]1c1n[nH]c(=O)cc1C2 ; CO[C@H]1CC[C@@H](Oc2ccc(-c3cc(F)c(NC(=O)N4[C@H]5CC[C@@H]4c4n[nH]c(=O)cc4C5)cc3Cl)cn2)CC1]

and enantiomers thereof, and mixtures of enantiomers thereof, including racemic mixtures, and pharmaceutically acceptable salts and solvates thereof.

Another aspect of the invention relates to a compound of formula (If), or a pharmaceutically acceptable salt or solvate thereof,

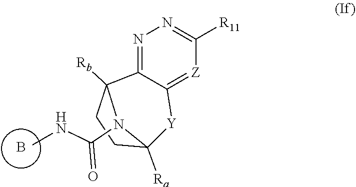

wherein:
R₁₁ is selected from H, alkyl, haloalkyl, halo, OH and O-alkyl; and
B, Y, Z, Rₐ and R_{b} are as defined hereinabove.

In one preferred embodiment, for compounds of formula (If), ring B is:
a monocyclic aromatic group; or
a monocyclic or bicyclic heteroaromatic group,
each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl and CO₂-alkyl, wherein said aryl, heteroaryl and O-aryl groups are each optionally further substituted by one or more groups independently selected from halo, alkyl and alkoxy;
Z is CR₁₂;
Y is CR₁₀R_{10′}, wherein R₁₀ and R_{10′} are each independently selected from H, F, alkyl, and haloalkyl;
Rₐ and R_{b} are each independently selected from H and alkyl; and
R₁₁ and R₁₂ are each independently selected from H, alkyl, haloalkyl, halo, OH and O-alkyl.

In one preferred embodiment, R₁₁ is selected from H, alkyl, haloalkyl, halo and O-alkyl.

In one preferred embodiment, R₁₁ is OH.

In one preferred embodiment, R₁₂ is selected from H, CF₃, Me, Cl, F, Br, OH and OMe. Even more preferably, R₁₂ is H, i.e. Z is CH.

Preferred definitions for B, Rₐ, R_{b}, Y and Z as defined above apply equally to compounds of formula (If).

Another aspect of the invention relates to a compound of formula (Ij) or a pharmaceutically acceptable salt or solvate thereof,

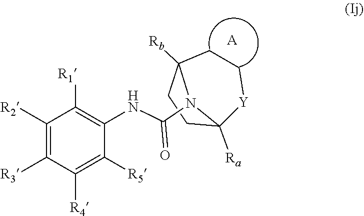

wherein ring A is selected from:

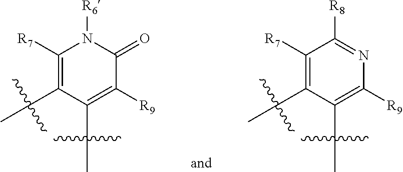

Y is CR₁₀R_{10′}, wherein R₁₀ and R_{10′} are each independently selected from H, F, alkyl, and haloalkyl; preferably Y is CH₂;
Rₐ and R_{b} are each independently selected from H and alkyl; preferably Rₐ and R_{b} are both H;
R_{1′}, R_{2′}, R_{4′} and R_{5′} are each independently selected from H, CN, alkyl, alkoxy, haloalkyl, OH and halo;
R_{3′} is a pyridinyl group substituted by one or more substituents selected from CN, haloalkyl, haloalkoxy, NHCO-alkyl, NR₁₃R_{13′}, SO₂-alkyl, O—(CH₂)_{q}-heterocycloalkyl, alkoxy-alkoxy, alkylamino-alkoxy, dialkylamino-alkoxy, alkoxy-alkyl and O—(CH₂)ₚ-cycloalkyl, wherein said cycloalkyl group is optionally substituted by one or more halo, alkyl or alkoxy groups, and wherein said pyridinyl is optionally further substituted by one or more substituents selected from halo, alkyl and alkoxy;
R_{6′} is H or alkyl, more preferably H;
R₇, R₈ and R₉ are each independently selected from H, halo and alkyl;
R₁₃ and R₁₃, are each independently selected from H, alkyl, and alkoxy-alkyl; and
p and q are each independently 0 to 3.

In one preferred embodiment:
R_{1′} and R_{4′} are selected from halo and haloalkyl;
R_{2′} and R_{5′} are H;
R_{3′} is:

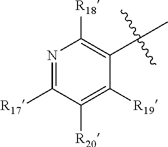

wherein:
   R_{17′} is selected from CN, haloalkyl, haloalkoxy, NHCO-alkyl, NR₁₃R_{13′}, SO₂-alkyl, O—(CH₂)_{q}-heterocycloalkyl, alkoxy-alkoxy, alkylamino-alkoxy, dialkylamino-alkoxy, alkoxy-alkyl and O—(CH₂)ₚ-cycloalkyl, wherein said cycloalkyl group is optionally substituted by one or more halo, alkyl or alkoxy groups;
   R_{18′} is selected from H and halo;
   R_{19′} is selected from H, alkoxy and alkoxy-alkyl; and
   R_{20′} is selected from H and halo.

More preferably, p and q are each independently 0 or 1.

In one preferred embodiment, R_{1′} and R_{4′} are both halo.

In one preferred embodiment, R_{1′} and R_{4′} are selected from Cl and F.

In one preferred embodiment, R_{1′} is F and R_{4′} is Cl.

In on preferred embodiment:
ring A is selected from:

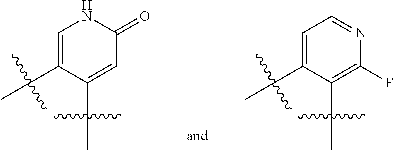 [SMILES: *c1c[nH]c(=O)cc1* ; *c1ccnc(F)c1*]

Y is CH₂;
Rₐ and R_{b} are both H;
R_{1′} is F and R_{4′} is Cl; and
R_{17′} is selected from haloalkyl, haloalkoxy, alkylamino-alkoxy, dialkylamino-alkoxy, O—(CH₂)_{q}-heterocycloalkyl, O—(CH₂)ₚ-cycloalkyl, wherein said cycloalkyl group is optionally substituted by one or more alkoxy groups;
p is 0 or 1;
q is 0 or 1; and
R_{18′}, R_{19′} and R_{20′} are all H.

In one preferred embodiment, ring A is:

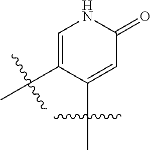 [SMILES: *c1c[nH]c(=O)cc1*]

In one preferred embodiment, ring A is:

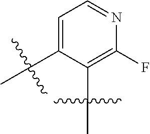 [SMILES: *c1ccnc(F)c1*]

In one preferred embodiment, R_{17′} is selected from haloalkyl and haloalkoxy, more preferably fluoroalkyl and fluoroalkoxy.

In one preferred embodiment, R_{17′} is selected from —O—CH₂CF₃, —O—CH₂CH₂CF₃ and —OCHF₂, —OCH₂CHF₂.

In one preferred embodiment, R_{17′} is selected from fluoroalkyl, fluoroalkoxy, methylamino-alkoxy, dimethylamino-alkoxy, (piperidin-1-yl)-alkoxy, O—(CH₂)ₚ-cyclopropyl, O—(CH₂)ₚ-cyclobutyl, wherein said cyclopropyl and cyclobutyl group is optionally substituted by an alkoxy group; and R_{13′}, R_{19′} and R_{20′} are all H.

In one preferred embodiment, R_{17′} is selected from CF₃, —O—CH₂CF₃, —O—CH₂CH₂CF₃, —OCHF₂, —OCH₂CHF₂, —OCH₂CH₂NHMe, —OCH₂CH₂NHMe, (piperidin-1-yl)-CH₂CH₂—O—, —O— cyclopropyl, —O—CH₂-cyclopropyl, methoxycyclobutyl-O—; and R_{13′}, R_{19′} and R_{20′} are all H.

In one preferred embodiment, the compound of formula (Ij) is selected from compounds 220, 222, 223, 239, 240, 241, 300, 301, 309, 312, 317, 321, 322, 324, 325, 332 and 334 described herein.

A further aspect of the invention relates to a compound selected from the following:

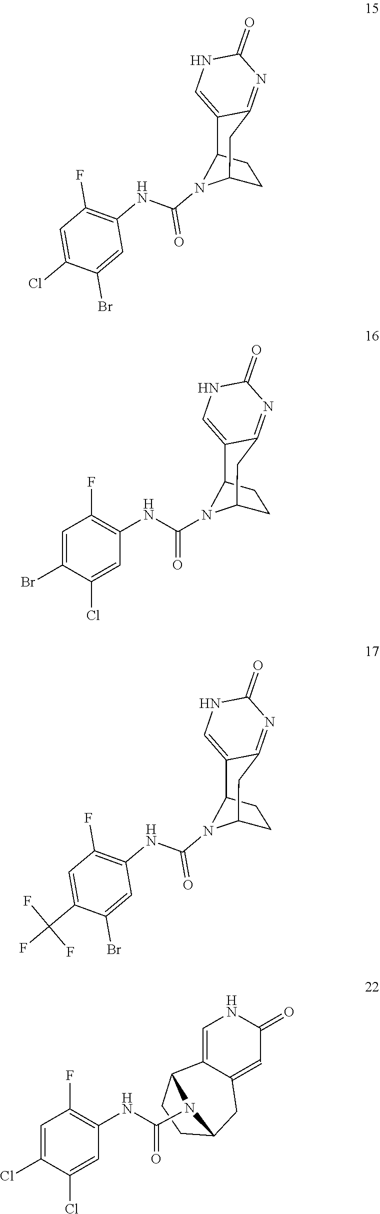 [SMILES: O=C(Nc1cc(Br)c(Cl)cc1F)N1C2CCC1c1c[nH]c(=O)nc1C2 ; O=C(Nc1cc(Cl)c(Br)cc1F)N1C2CCC1c1c[nH]c(=O)nc1C2 ; O=C(Nc1cc(Br)c(C(F)(F)F)cc1F)N1C2CCC1c1c[nH]c(=O)nc1C2 ; O=C(Nc1cc(Cl)c(Cl)cc1F)N1[C@H]2CC[C@@H]1c1c[nH]c(=O)cc1C2]

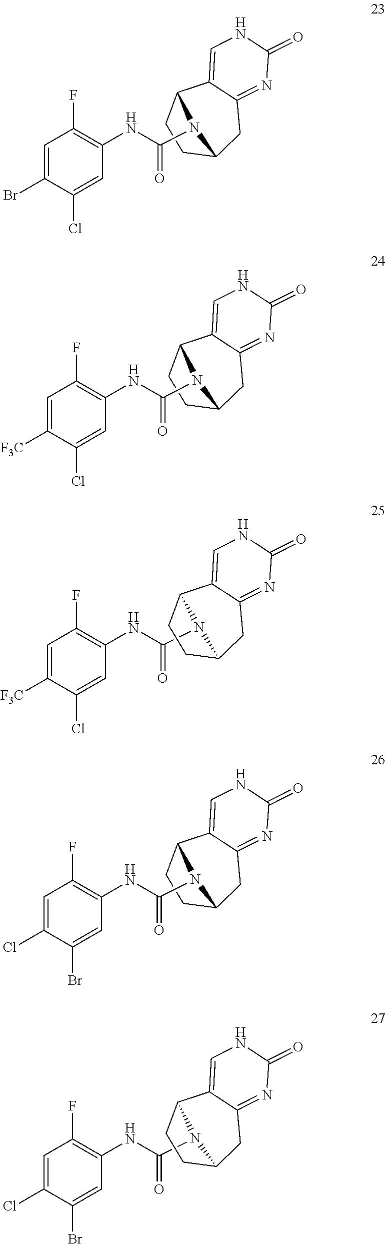 [SMILES: O=C(Nc1cc(Cl)c(Br)cc1F)N1[C@H]2CC[C@@H]1c1c[nH]c(=O)nc1C2 ; O=C(Nc1cc(Cl)c(C(F)(F)F)cc1F)N1[C@H]2CC[C@@H]1c1c[nH]c(=O)nc1C2 ; O=C(Nc1cc(Cl)c(C(F)(F)F)cc1F)N1[C@@H]2CC[C@H]1c1c[nH]c(=O)nc1C2 ; O=C(Nc1cc(Br)c(Cl)cc1F)N1[C@H]2CC[C@@H]1c1c[nH]c(=O)nc1C2 ; O=C(Nc1cc(Br)c(Cl)cc1F)N1[C@@H]2CC[C@H]1c1c[nH]c(=O)nc1C2]

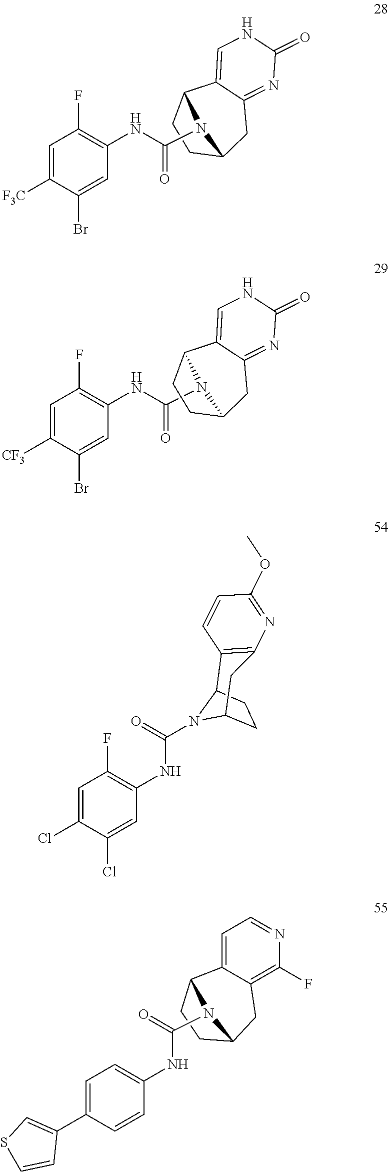 [SMILES: O=C(Nc1cc(Br)c(C(F)(F)F)cc1F)N1[C@H]2CC[C@@H]1c1c[nH]c(=O)nc1C2 ; O=C(Nc1cc(Br)c(C(F)(F)F)cc1F)N1[C@@H]2CC[C@H]1c1c[nH]c(=O)nc1C2 ; COc1ccc2c(n1)CC1CCC2N1C(=O)Nc1cc(Cl)c(Cl)cc1F ; O=C(Nc1ccc(-c2ccsc2)cc1)N1[C@H]2CC[C@@H]1c1ccnc(F)c1C2]

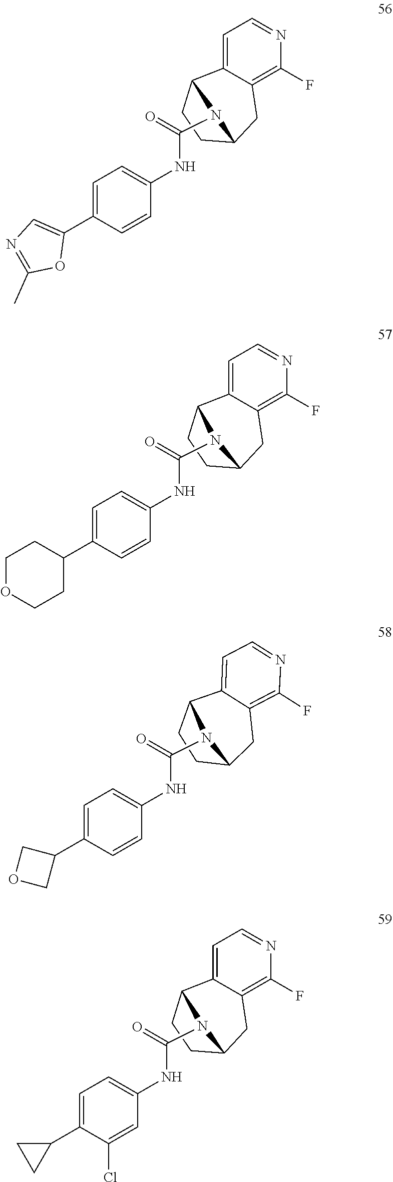 [SMILES: Cc1ncc(-c2ccc(NC(=O)N3[C@H]4CC[C@@H]3c3ccnc(F)c3C4)cc2)o1 ; O=C(Nc1ccc(C2CCOCC2)cc1)N1[C@H]2CC[C@@H]1c1ccnc(F)c1C2 ; O=C(Nc1ccc(C2COC2)cc1)N1[C@H]2CC[C@@H]1c1ccnc(F)c1C2 ; O=C(Nc1ccc(C2CC2)c(Cl)c1)N1[C@H]2CC[C@@H]1c1ccnc(F)c1C2]

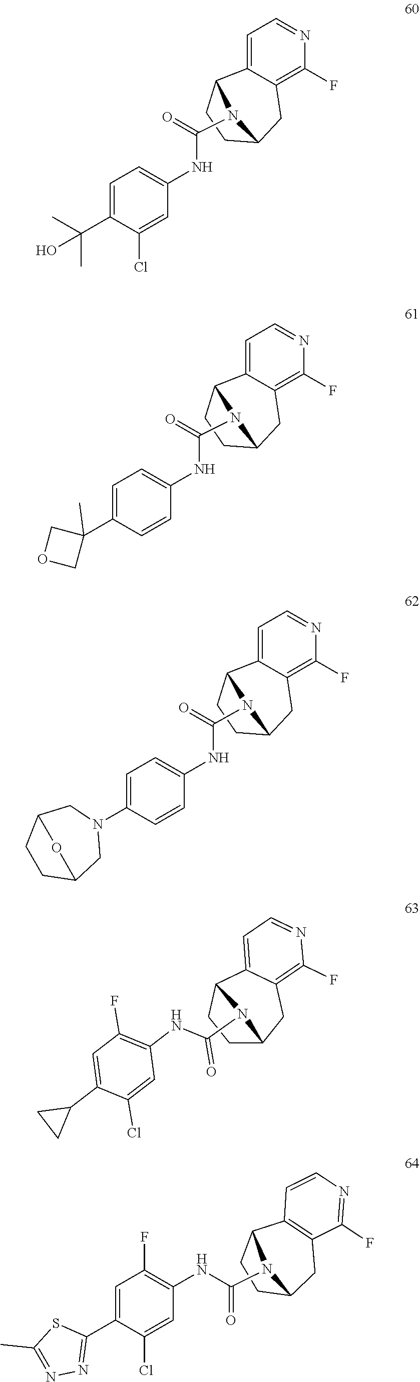 [SMILES: CC(C)(O)c1ccc(NC(=O)N2[C@H]3CC[C@@H]2c2ccnc(F)c2C3)cc1Cl ; CC1(c2ccc(NC(=O)N3[C@H]4CC[C@@H]3c3ccnc(F)c3C4)cc2)COC1 ; O=C(Nc1ccc(N2CC3CCC(C2)O3)cc1)N1[C@H]2CC[C@@H]1c1ccnc(F)c1C2 ; O=C(Nc1cc(Cl)c(C2CC2)cc1F)N1[C@H]2CC[C@@H]1c1ccnc(F)c1C2 ; Cc1nnc(-c2cc(F)c(NC(=O)N3[C@H]4CC[C@@H]3c3ccnc(F)c3C4)cc2Cl)s1]

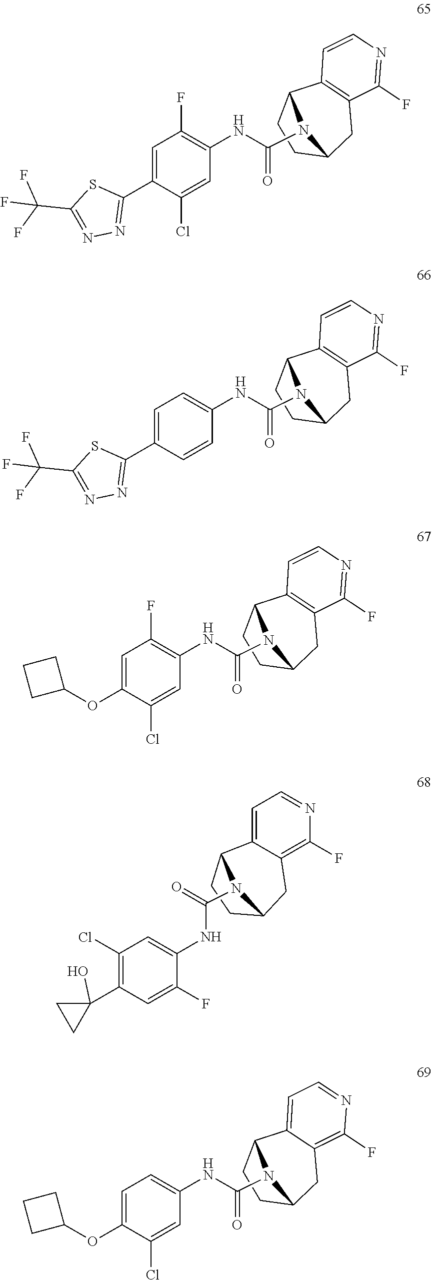 [SMILES: O=C(Nc1cc(Cl)c(-c2nnc(C(F)(F)F)s2)cc1F)N1[C@H]2CC[C@@H]1c1ccnc(F)c1C2 ; O=C(Nc1ccc(-c2nnc(C(F)(F)F)s2)cc1)N1[C@H]2CC[C@@H]1c1ccnc(F)c1C2 ; O=C(Nc1cc(Cl)c(OC2CCC2)cc1F)N1[C@H]2CC[C@@H]1c1ccnc(F)c1C2 ; O=C(Nc1cc(Cl)c(C2(O)CC2)cc1F)N1[C@H]2CC[C@@H]1c1ccnc(F)c1C2 ; O=C(Nc1ccc(OC2CCC2)c(Cl)c1)N1[C@H]2CC[C@@H]1c1ccnc(F)c1C2]

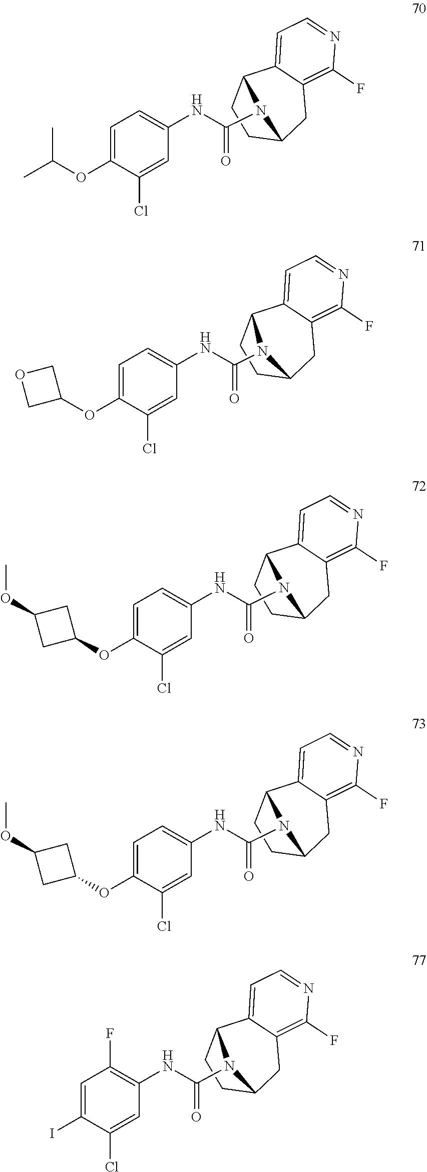 [SMILES: CC(C)Oc1ccc(NC(=O)N2[C@H]3CC[C@@H]2c2ccnc(F)c2C3)cc1Cl ; O=C(Nc1ccc(OC2COC2)c(Cl)c1)N1[C@H]2CC[C@@H]1c1ccnc(F)c1C2 ; CO[C@H]1C[C@@H](Oc2ccc(NC(=O)N3[C@H]4CC[C@@H]3c3ccnc(F)c3C4)cc2Cl)C1 ; CO[C@H]1C[C@H](Oc2ccc(NC(=O)N3[C@H]4CC[C@@H]3c3ccnc(F)c3C4)cc2Cl)C1 ; O=C(Nc1cc(Cl)c(I)cc1F)N1[C@H]2CC[C@@H]1c1ccnc(F)c1C2]

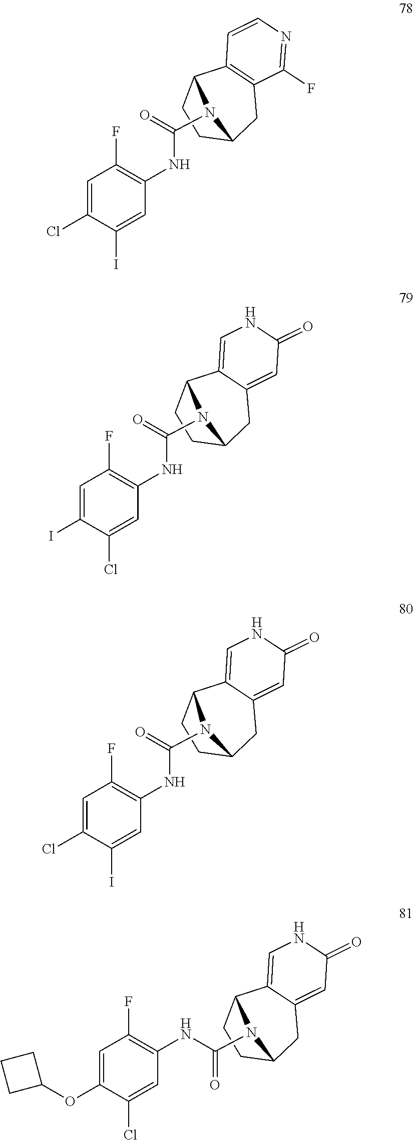 [SMILES: O=C(Nc1cc(I)c(Cl)cc1F)N1[C@H]2CC[C@@H]1c1ccnc(F)c1C2 ; O=C(Nc1cc(Cl)c(I)cc1F)N1[C@H]2CC[C@@H]1c1c[nH]c(=O)cc1C2 ; O=C(Nc1cc(I)c(Cl)cc1F)N1[C@H]2CC[C@@H]1c1c[nH]c(=O)cc1C2 ; O=C(Nc1cc(Cl)c(OC2CCC2)cc1F)N1[C@H]2CC[C@@H]1c1c[nH]c(=O)cc1C2]

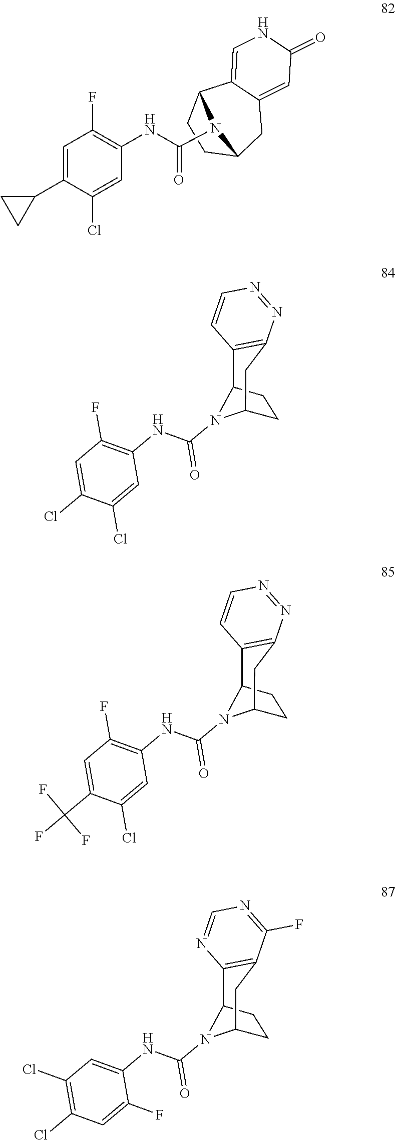 [SMILES: O=C(Nc1cc(Cl)c(C2CC2)cc1F)N1[C@H]2CC[C@@H]1c1c[nH]c(=O)cc1C2 ; O=C(Nc1cc(Cl)c(Cl)cc1F)N1C2CCC1c1ccnnc1C2 ; O=C(Nc1cc(Cl)c(C(F)(F)F)cc1F)N1C2CCC1c1ccnnc1C2 ; O=C(Nc1cc(Cl)c(Cl)cc1F)N1C2CCC1c1ncnc(F)c1C2]

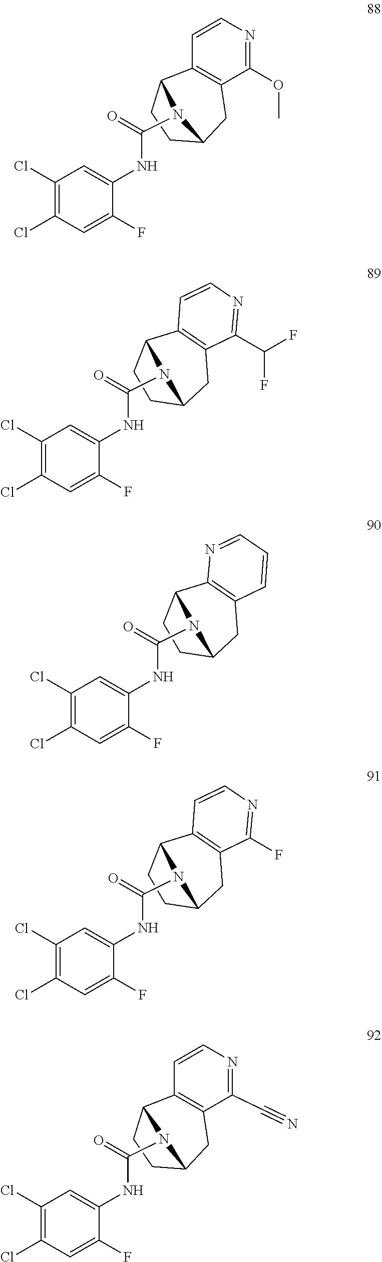 [SMILES: COc1nccc2c1C[C@@H]1CC[C@H]2N1C(=O)Nc1cc(Cl)c(Cl)cc1F ; O=C(Nc1cc(Cl)c(Cl)cc1F)N1[C@H]2CC[C@@H]1c1ccnc(C(F)F)c1C2 ; O=C(Nc1cc(Cl)c(Cl)cc1F)N1[C@H]2CC[C@@H]1c1ncccc1C2 ; O=C(Nc1cc(Cl)c(Cl)cc1F)N1[C@H]2CC[C@@H]1c1ccnc(F)c1C2 ; N#Cc1nccc2c1C[C@@H]1CC[C@H]2N1C(=O)Nc1cc(Cl)c(Cl)cc1F]

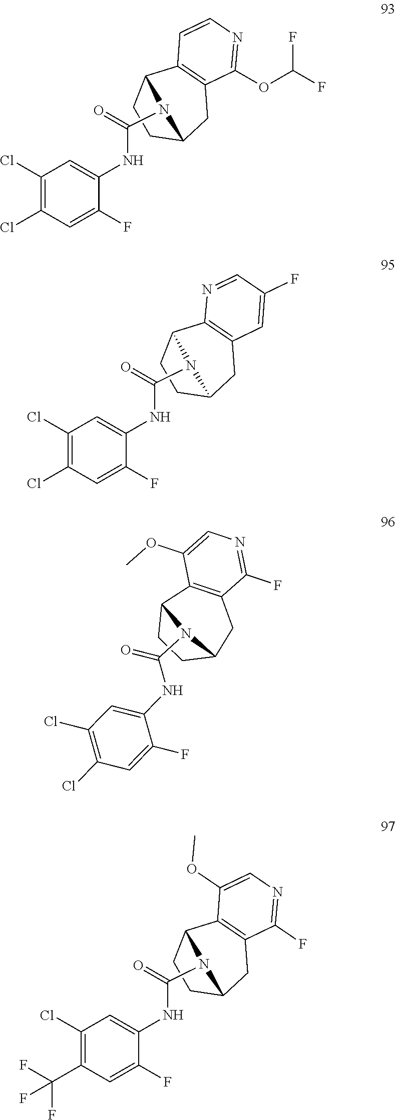 [SMILES: O=C(Nc1cc(Cl)c(Cl)cc1F)N1[C@H]2CC[C@@H]1c1ccnc(OC(F)F)c1C2 ; O=C(Nc1cc(Cl)c(Cl)cc1F)N1[C@@H]2CC[C@H]1c1ncc(F)cc1C2 ; COc1cnc(F)c2c1[C@H]1CC[C@@H](C2)N1C(=O)Nc1cc(Cl)c(Cl)cc1F ; COc1cnc(F)c2c1[C@H]1CC[C@@H](C2)N1C(=O)Nc1cc(Cl)c(C(F)(F)F)cc1F]

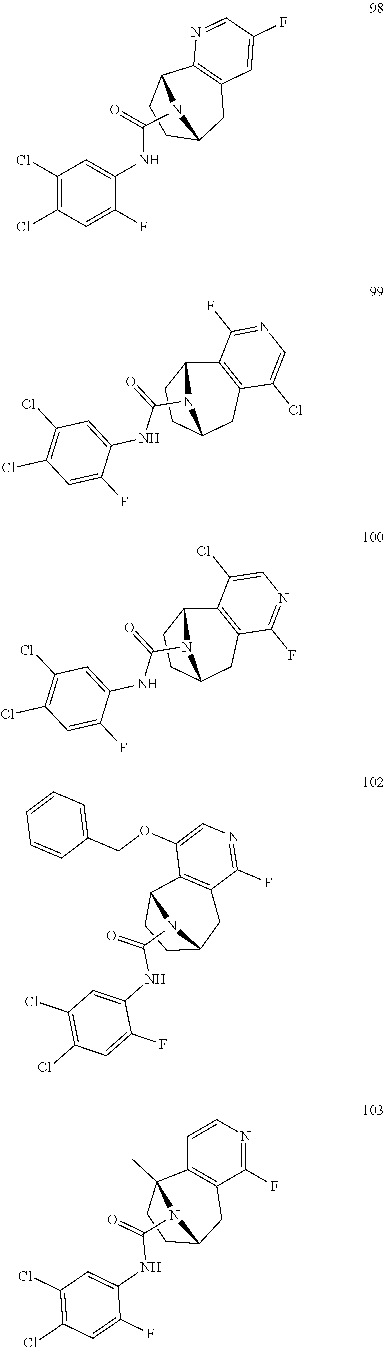 [SMILES: O=C(Nc1cc(Cl)c(Cl)cc1F)N1[C@H]2CC[C@@H]1c1ncc(F)cc1C2 ; O=C(Nc1cc(Cl)c(Cl)cc1F)N1[C@H]2CC[C@@H]1c1c(F)ncc(Cl)c1C2 ; O=C(Nc1cc(Cl)c(Cl)cc1F)N1[C@H]2CC[C@@H]1c1c(Cl)cnc(F)c1C2 ; O=C(Nc1cc(Cl)c(Cl)cc1F)N1[C@H]2CC[C@@H]1c1c(OCc3ccccc3)cnc(F)c1C2 ; C[C@@]12CC[C@@H](Cc3c1ccnc3F)N2C(=O)Nc1cc(Cl)c(Cl)cc1F]

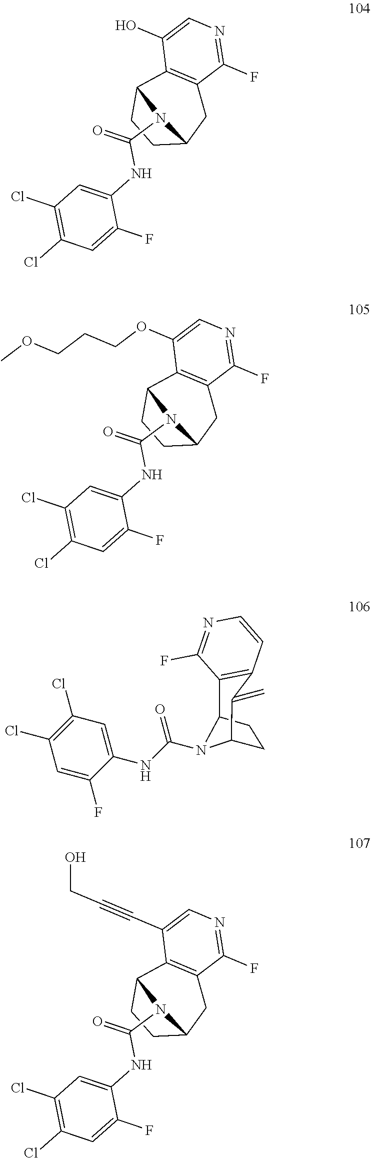 [SMILES: O=C(Nc1cc(Cl)c(Cl)cc1F)N1[C@H]2CC[C@@H]1c1c(O)cnc(F)c1C2 ; COCCCOc1cnc(F)c2c1[C@H]1CC[C@@H](C2)N1C(=O)Nc1cc(Cl)c(Cl)cc1F ; C=C1c2ccnc(F)c2C2CCC1N2C(=O)Nc1cc(Cl)c(Cl)cc1F ; O=C(Nc1cc(Cl)c(Cl)cc1F)N1[C@H]2CC[C@@H]1c1c(C#CCO)cnc(F)c1C2]

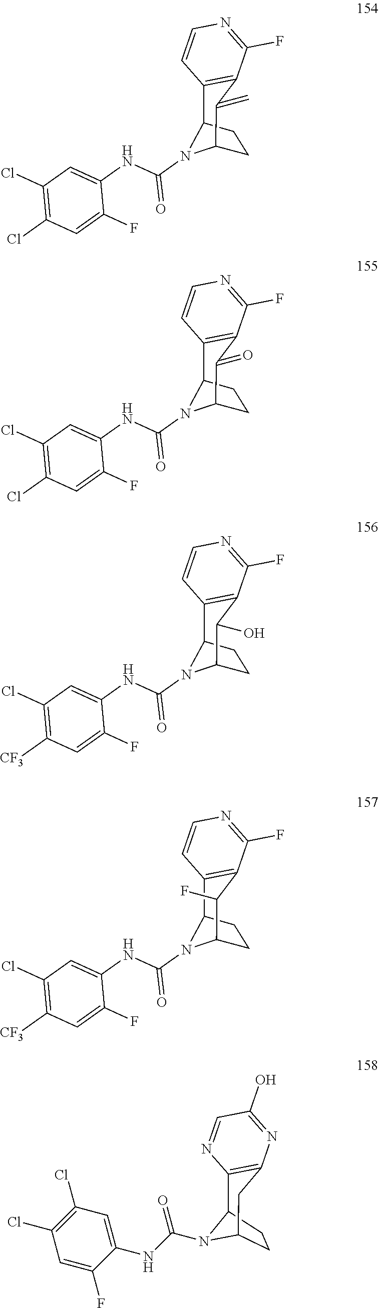 [SMILES: C=C1c2c(ccnc2F)C2CCC1N2C(=O)Nc1cc(Cl)c(Cl)cc1F ; O=C1c2c(ccnc2F)C2CCC1N2C(=O)Nc1cc(Cl)c(Cl)cc1F ; O=C(Nc1cc(Cl)c(C(F)(F)F)cc1F)N1C2CCC1C(O)c1c2ccnc1F ; O=C(Nc1cc(Cl)c(C(F)(F)F)cc1F)N1C2CCC1C(F)c1c2ccnc1F ; O=C(Nc1cc(Cl)c(Cl)cc1F)N1C2CCC1c1ncc(O)nc1C2]

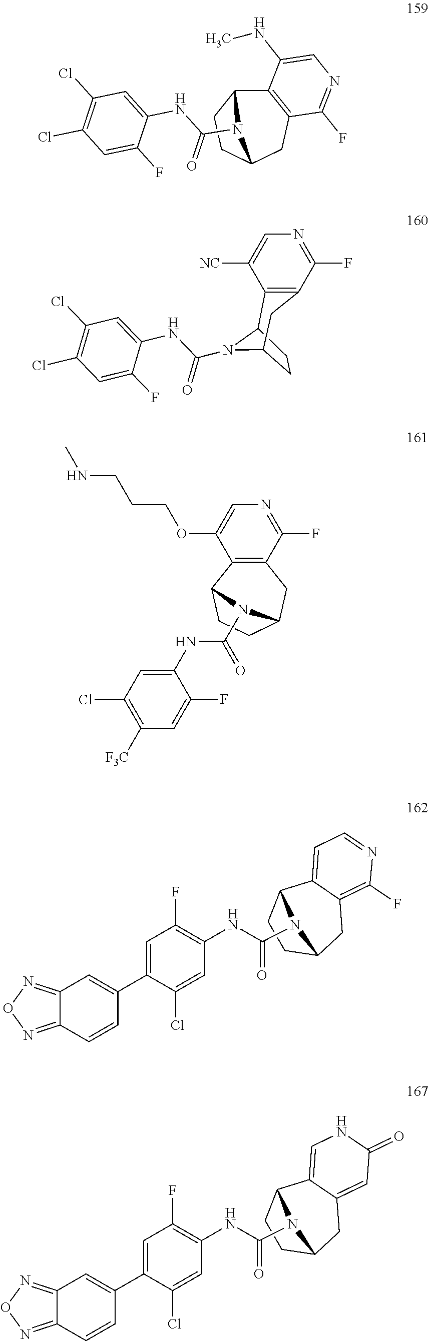

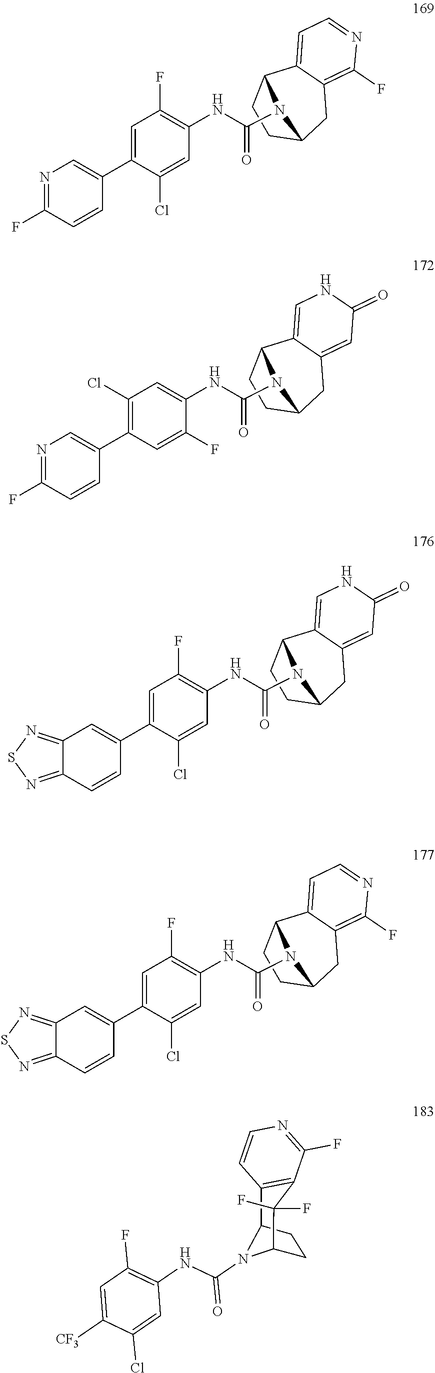 [SMILES: O=C(Nc1cc(Cl)c(-c2ccc(F)nc2)cc1F)N1[C@H]2CC[C@@H]1c1ccnc(F)c1C2 ; O=C(Nc1cc(Cl)c(-c2ccc(F)nc2)cc1F)N1[C@H]2CC[C@@H]1c1c[nH]c(=O)cc1C2 ; O=C(Nc1cc(Cl)c(-c2ccc3nsnc3c2)cc1F)N1[C@H]2CC[C@@H]1c1c[nH]c(=O)cc1C2 ; O=C(Nc1cc(Cl)c(-c2ccc3nsnc3c2)cc1F)N1[C@H]2CC[C@@H]1c1ccnc(F)c1C2 ; O=C(Nc1cc(Cl)c(C(F)(F)F)cc1F)N1C2CCC1C(F)(F)c1c2ccnc1F]

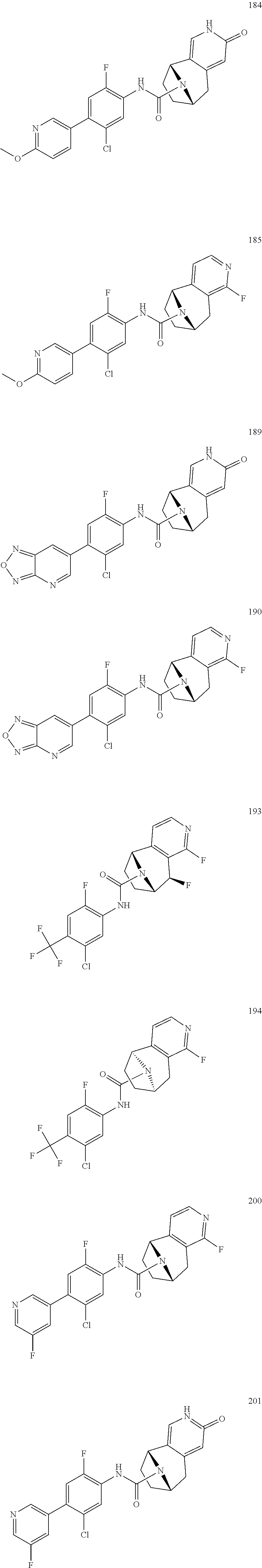

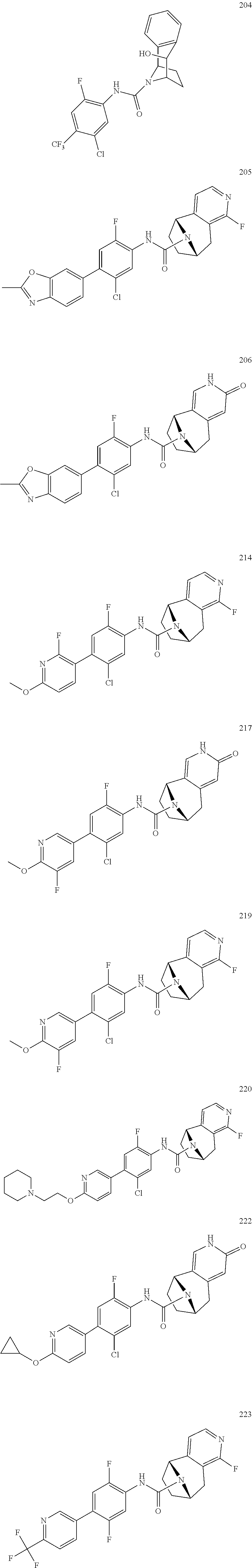

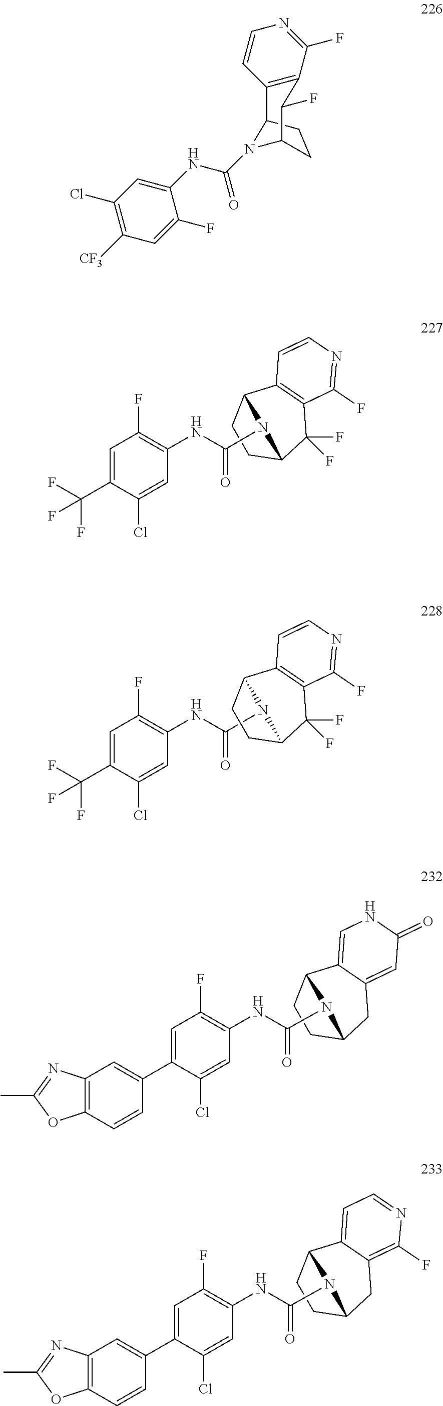 [SMILES: O=C(Nc1cc(Cl)c(C(F)(F)F)cc1F)N1C2CCC1C(F)c1c2ccnc1F ; O=C(Nc1cc(Cl)c(C(F)(F)F)cc1F)N1[C@@H]2CC[C@H]1C(F)(F)c1c2ccnc1F ; O=C(Nc1cc(Cl)c(C(F)(F)F)cc1F)N1[C@H]2CC[C@@H]1C(F)(F)c1c2ccnc1F ; Cc1nc2cc(-c3cc(F)c(NC(=O)N4[C@H]5CC[C@@H]4c4c[nH]c(=O)cc4C5)cc3Cl)ccc2o1 ; Cc1nc2cc(-c3cc(F)c(NC(=O)N4[C@H]5CC[C@@H]4c4ccnc(F)c4C5)cc3Cl)ccc2o1]

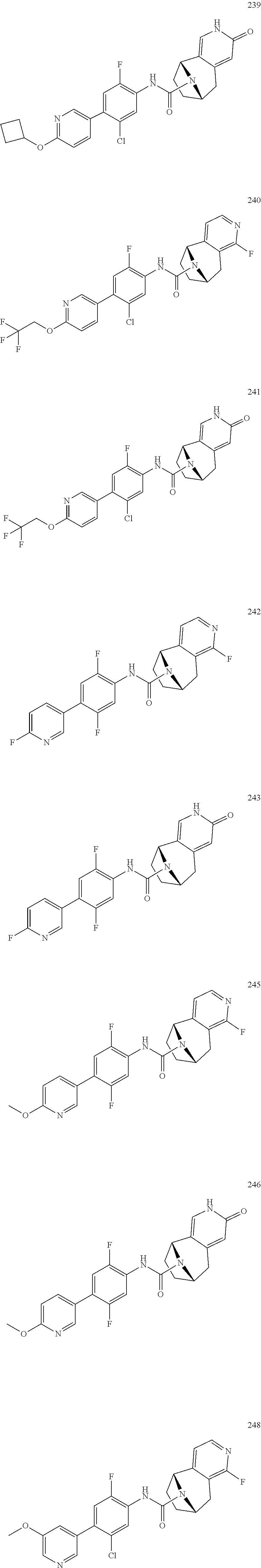

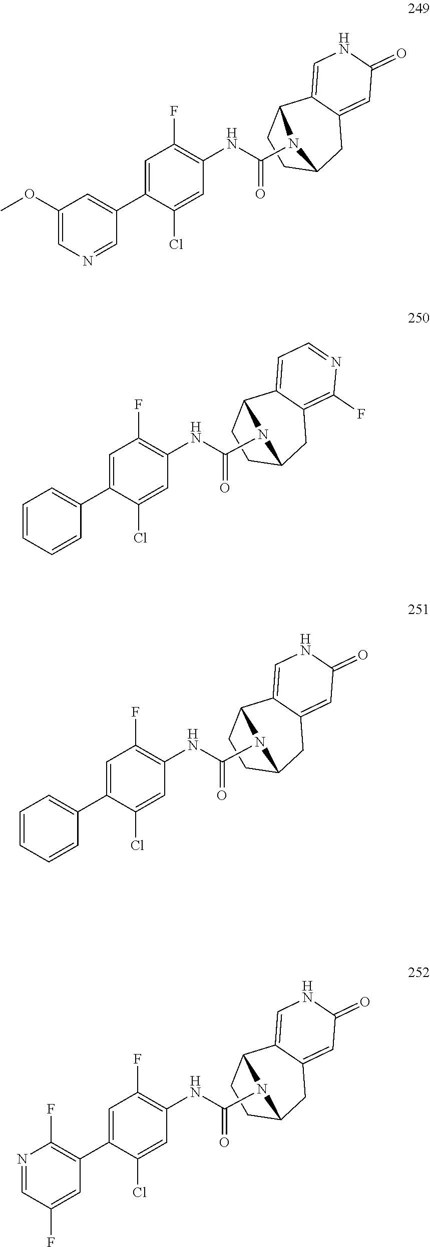 [SMILES: COc1cncc(-c2cc(F)c(NC(=O)N3[C@H]4CC[C@@H]3c3c[nH]c(=O)cc3C4)cc2Cl)c1 ; O=C(Nc1cc(Cl)c(-c2ccccc2)cc1F)N1[C@H]2CC[C@@H]1c1ccnc(F)c1C2 ; O=C(Nc1cc(Cl)c(-c2ccccc2)cc1F)N1[C@H]2CC[C@@H]1c1c[nH]c(=O)cc1C2 ; O=C(Nc1cc(Cl)c(-c2cc(F)cnc2F)cc1F)N1[C@H]2CC[C@@H]1c1c[nH]c(=O)cc1C2]

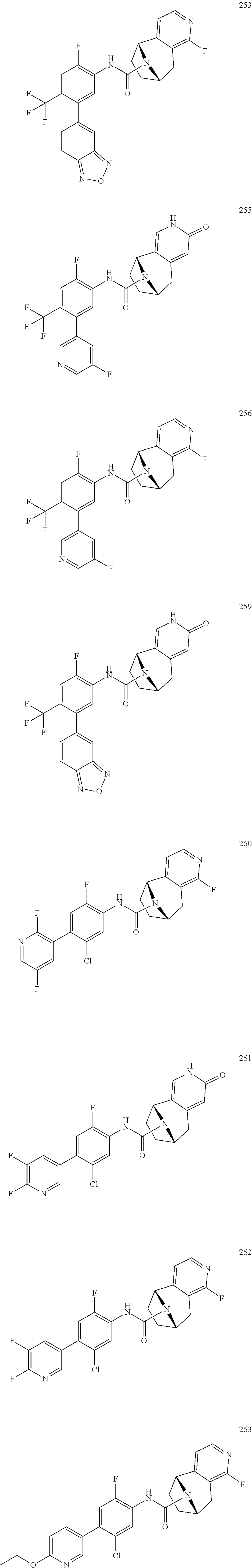

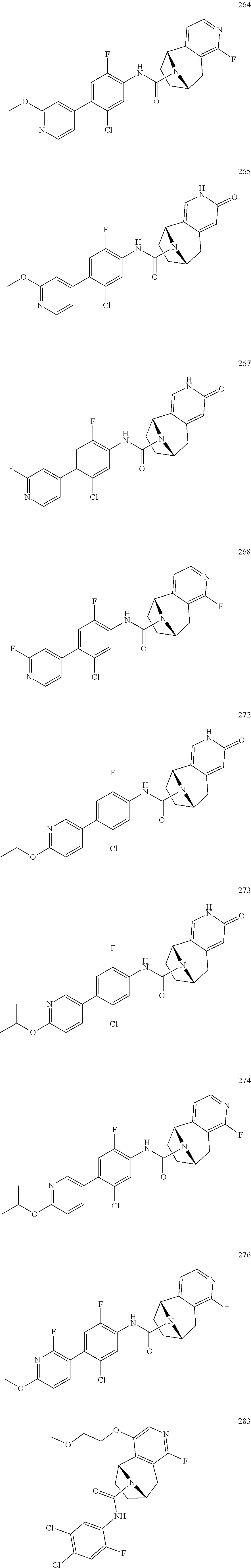

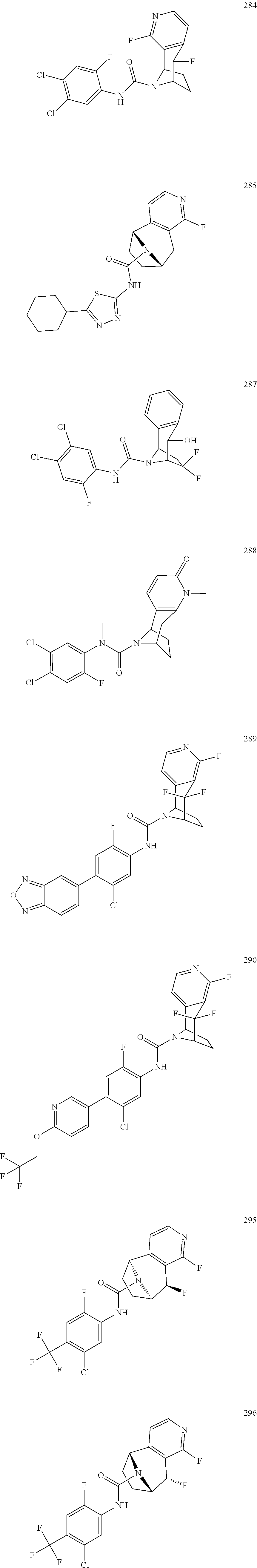

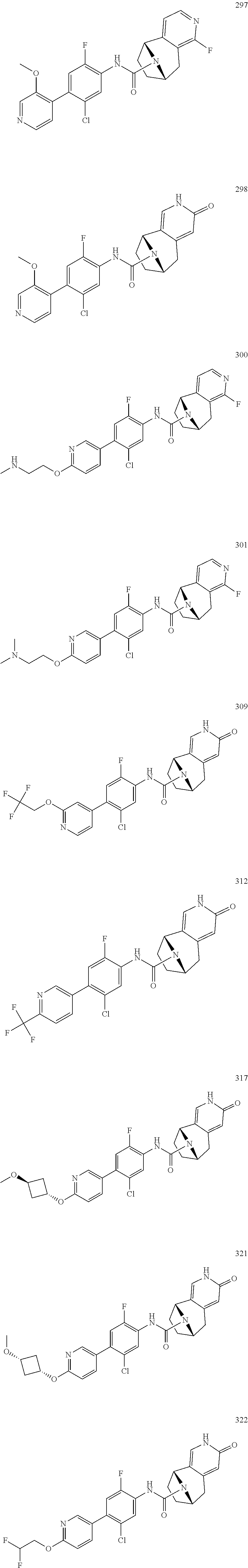

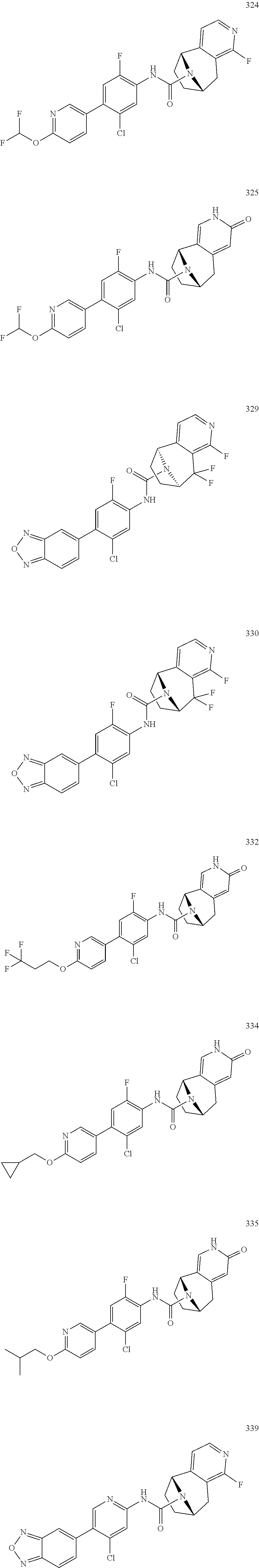

and enantiomers thereof, and mixtures of enantiomers thereof, including racemic mixtures, and pharmaceutically acceptable salts and solvates thereof.

In one preferred embodiment, the compound of the invention is selected from the following:

| | |
|---|---|
| 1 | (±)-N-(5-Chloro-2-fluoro-4-(trifluoromethyl)phenyl)-3-oxo-3,5,6,7,8,9- |
| | hexahydro-2H-6,9-epiminocyclohepta[c]pyridazine-10-carboxamide |
| 2 | (±)-N-(4,5-dichloro-2-fluorophenyl)-3-oxo-3,5,6,7,8,9-hexahydro-2H-6,9- |
| | epiminocyclohepta[c]pyridazine-10-carboxamide |
| 3 | (±)-N-(3-bromo-4-(trifluoromethyl)phenyl)-3-oxo-3,5,6,7,8,9-hexahydro-2H- |
| | 6,9-epiminocyclohepta[c]pyridazine-10-carboxamide |
| 4 | (±)-N-(4-bromo-3-chlorophenyl)-3-oxo-3,5,6,7,8,9-hexahydro-2H-6,9- |
| | epiminocyclohepta[c]pyridazine-10-carboxamide |
| 5 | (±)-N-(3-bromo-4-chlorophenyl)-3-oxo-3,5,6,7,8,9-hexahydro-2H-6,9- |
| | epiminocyclohepta[c]pyridazine-10-carboxamide |
| 6 | (±)-N-(5-bromo-2-fluoro-4-(trifluoromethyl)phenyl)-3-oxo-3,5,6,7,8,9- |
| | hexahydro-2H-6,9-epiminocyclohepta[c]pyridazine-10-carboxamide |
| 7 | (±)-N-(5-bromo-4-chloro-2-fluorophenyl)-3-oxo-3,5,6,7,8,9-hexahydro-2H- |
| | 6,9-epiminocyclohepta[c]pyridazine-10-carboxamide |
| 8 | (±)-N-(4-bromo-5-chloro-2-fluorophenyl)-3-oxo-3,5,6,7,8,9-hexahydro-2H- |
| | 6,9-epiminocyclohepta[c]pyridazine-10-carboxamide |
| 9 | (±)-N-(3-chloro-4-(trifluoromethoxy)phenyl)-3-oxo-3,5,6,7,8,9-hexahydro-2H- |
| | 6,9-epiminocyclohepta[c]pyridazine-10-carboxamide |
| 10 | (±)-N-(4-bromo-3-(trifluoromethyl)phenyl)-3-oxo-3,5,6,7,8,9-hexahydro-2H- |
| | 6,9-epiminocyclohepta[c]pyridazine-10-carboxamide |
| 11 | (±)-N-(3-cyano-4-(trifluoromethyl)phenyl)-3-oxo-3,5,6,7,8,9-hexahydro-2H- |
| | 6,9-epiminocyclohepta[c]pyridazine-10-carboxamide |
| 12 | (±)-N-(4-chloro-5-(trifluoromethyl)pyridin-2-yl)-3-oxo-3,5,6,7,8,9-hexahydro- |
| | 2H-6,9-epiminocyclohepta[c]pyridazine-10-carboxamide |
| 13 | (±)-N-(3-chloro-4-isopropoxyphenyl)-3-oxo-3,5,6,7,8,9-hexahydro-2H-6,9- |
| | epiminocyclohepta[c]pyridazine-10-carboxamide |
| 14 | (±)-N-(3-chloro-4-cyclobutoxyphenyl)-3-oxo-3,5,6,7,8,9-hexahydro-2H-6,9- |
| | epiminocyclohepta[c]pyridazine-10-carboxamide |
| 15 | (±)-N-(5-bromo-4-chloro-2-fluorophenyl)-2-oxo-3,5,6,7,8,9-hexahydro-2H- |
| | 5,8-epiminocyclohepta[d]pyrimidine-10-carboxamide |
| 16 | (±)-N-(4-bromo-5-chloro-2-fluorophenyl)-2-oxo-3,5,6,7,8,9-hexahydro-2H- |
| | 5,8-epiminocyclohepta[d]pyrimidine-10-carboxamide |
| 17 | (±)-N-(5-bromo-2-fluoro-4-(trifluoromethyl)phenyl)-2-oxo-3,5,6,7,8,9- |
| | hexahydro-2H-5,8-epiminocyclohepta[d]pyrimidine-10-carboxamide |
| 18 | (6S,9R)-N-(5-chloro-2-fluoro-4-(trifluoromethyl)phenyl)-3-oxo-3,5,6,7,8,9- |
| | hexahydro-2H-6,9-epiminocyclohepta[c]pyridazine-10-carboxamide |
| 19 | (6R,9S)-N-(5-chloro-2-fluoro-4-(trifluoromethyl)phenyl)-3-oxo-3,5,6,7,8,9- |
| | hexahydro-2H-6,9-epiminocyclohepta[c]pyridazine-10-carboxamide |
| 20 | (6S,9R)-N-(4,5-dichloro-2-fluorophenyl)-3-oxo-3,5,6,7,8,9-hexahydro-2H-6,9- |
| | epiminocyclohepta[c]pyridazine-10-carboxamide |
| 21 | (6R,9S)-N-(4,5-dichloro-2-fluorophenyl)-3-oxo-3,5,6,7,8,9-hexahydro-2H-6,9- |
| | epiminocyclohepta[c]pyridazine-10-carboxamide |
| 22 | (5R,8S)-N-(4,5-dichloro-2-fluorophenyl)-2-oxo-3,5,6,7,8,9-hexahydro-2H-5,8- |
| | epiminocyclohepta[d]pyrimidine-10-carboxamide |
| 23 | (5R,8S)-N-(4-bromo-5-chloro-2-fluorophenyl)-2-oxo-3,5,6,7,8,9-hexahydro- |
| | 2H-5,8-epiminocyclohepta[d]pyrimidine-10-carboxamide |
| 24 | (5R,8S)-N-(5-chloro-2-fluoro-4-(trifluoromethyl)phenyl)-2-oxo-3,5,6,7,8,9- |
| | hexahydro-2H-5,8-epiminocyclohepta[d]pyrimidine-10-carboxamide |
| 25 | (5S,8R)-N-(5-chloro-2-fluoro-4-(trifluoromethyl)phenyl)-2-oxo-3,5,6,7,8,9- |
| | hexahydro-2H-5,8-epiminocyclohepta[d]pyrimidine-10-carboxamide |
| 26 | (5R,8S)-N-(5-bromo-4-chloro-2-fluorophenyl)-2-oxo-3,5,6,7,8,9-hexahydro- |
| | 2H-5,8-epiminocyclohepta[d]pyrimidine-10-carboxamide |
| 27 | (5S,8R)-N-(5-bromo-4-chloro-2-fluorophenyl)-2-oxo-3,5,6,7,8,9-hexahydro- |
| | 2H-5,8-epiminocyclohepta[d]pyrimidine-10-carboxamide |
| 28 | (5R,8S)-N-(5-bromo-2-fluoro-4-(trifluoromethyl)phenyl)-2-oxo-3,5,6,7,8,9- |
| | hexahydro-2H-5,8-epiminocyclohepta[d]pyrimidine-10-carboxamide |
| 29 | (5S,8R)-N-(5-bromo-2-fluoro-4-(trifluoromethyl)phenyl)-2-oxo-3,5,6,7,8,9- |
| | hexahydro-2H-5,8-epiminocyclohepta[d]pyrimidine-10-carboxamide |
| 30 | (6R,9S)-N-(3-Bromo-4-(trifluoromethyl)phenyl)-3-oxo-3,5,6,7,8,9-hexahydro- |
| | 2H-6,9-epiminocyclohepta[c]pyridazine-10-carboxamide |
| 31 | (6S,9R)-N-(3-Bromo-4-(trifluoromethyl)phenyl)-3-oxo-3,5,6,7,8,9-hexahydro- |
| | 2H-6,9-epiminocyclohepta[c]pyridazine-10-carboxamide |
| 32 | (6S,9R)-N-(4-Bromo-5-chloro-2-fluorophenyl)-3-oxo-3,5,6,7,8,9-hexahydro- |
| | 2H-6,9-epiminocyclohepta[c]pyridazine-10-carboxamide |
| 33 | (6R,9S)-N-(4-Bromo-5-chloro-2-fluorophenyl)-3-oxo-3,5,6,7,8,9-hexahydro- |
| | 2H-6,9-epiminocyclohepta[c]pyridazine-10-carboxamide |
| 34 | (6R,9S)-N-(5-Bromo-2-fluoro-4-(trifluoromethyl)phenyl)-3-oxo-3,5,6,7,8,9- |
| | hexahydro-2H-6,9-epiminocyclohepta[c]pyridazine-10-carboxamide |
| 35 | (6S,9R)-N-(5-Bromo-2-fluoro-4-(trifluoromethyl)phenyl)-3-oxo-3,5,6,7,8,9- |
| | hexahydro-2H-6,9-epiminocyclohepta[c]pyridazine-10-carboxamide |
| 36 | (±)-N-(3-Chloro-4-((1s,3S)-3-methoxycyclobutoxy)phenyl)-3-oxo-3,5,6,7,8,9- |
| | hexahydro-2H-6,9-epiminocyclohepta[c]pyridazine-10-carboxamide |
| 37 | (6S,9R)-N-(5-Bromo-4-chloro-2-fluorophenyl)-3-oxo-3,5,6,7,8,9-hexahydro- |
| | 2H-6,9-epiminocyclohepta[c]pyridazine-10-carboxamide |
| 38 | (6R,9S)-N-(5-Bromo-4-chloro-2-fluorophenyl)-3-oxo-3,5,6,7,8,9-hexahydro- |
| | 2H-6,9-epiminocyclohepta[c]pyridazine-10-carboxamide |
| 39 | (6S,9R)-N-(4-Bromo-3-chlorophenyl)-3-oxo-3,5,6,7,8,9-hexahydro-2H-6,9- |
| | epiminocyclohepta[c]pyridazine-10-carboxamide |
| 40 | (6R,9S)-N-(4-Bromo-3-chlorophenyl)-3-oxo-3,5,6,7,8,9-hexahydro-2H-6,9- |
| | epiminocyclohepta[c]pyridazine-10-carboxamide |
| 41 | (6S,9R)-N-(3-Cyano-4-(trifluoromethyl)phenyl)-3-oxo-3,5,6,7,8,9-hexahydro- |
| | 2H-6,9-epiminocyclohepta[c]pyridazine-10-carboxamide |
| 42 | (6S,9R)-N-(5-Chloro-4-cyclopropyl-2-fluorophenyl)-3-oxo-3,5,6,7,8,9- |
| | hexahydro-2H-6,9-epiminocyclohepta[c]pyridazine-10-carboxamide |
| 43 | (±)-N-(4-chloro-5-cyanopyridin-2-yl)-3-oxo-3,5,6,7,8,9-hexahydro-2H-6,9- |
| | epiminocyclohepta[c]pyridazine-10-carboxamide |
| 44 | (±)-3-Oxo-N-(5-(trifluoromethyl)84soxazole-3-yl)-3,5,6,7,8,9-hexahydro-2H- |
| | 6,9-epiminocyclohepta[c]pyridazine-10-carboxamide |
| 45 | (6R,9S)-N-(3-Chloro-4-(trifluoromethoxy)phenyl)-3-oxo-3,5,6,7,8,9- |
| | hexahydro-2H-6,9-epiminocyclohepta[c]pyridazine-10-carboxamide |
| 46 | (6S,9R)-N-(3-Chloro-4-(trifluoromethoxy)phenyl)-3-oxo-3,5,6,7,8,9- |
| | hexahydro-2H-6,9-epiminocyclohepta[c]pyridazine-10-carboxamide |
| 47 | (6S,9R)-N-(4-Bromo-3-(trifluoromethyl)phenyl)-3-oxo-3,5,6,7,8,9-hexahydro- |
| | 2H-6,9-epiminocyclohepta[c]pyridazine-10-carboxamide |
| 48 | (6R,9S)-N-(4-Bromo-3-(trifluoromethyl)phenyl)-3-oxo-3,5,6,7,8,9-hexahydro- |
| | 2H-6,9-epiminocyclohepta[c]pyridazine-10-carboxamide |
| 49 | (6S,9R)-N-(3-Bromo-4-chlorophenyl)-3-oxo-3,5,6,7,8,9-hexahydro-2H-6,9- |
| | epiminocyclohepta[c]pyridazine-10-carboxamide |
| 50 | (6R,9S)-N-(3-Bromo-4-chlorophenyl)-3-oxo-3,5,6,7,8,9-hexahydro-2H-6,9- |
| | epiminocyclohepta[c]pyridazine-10-carboxamide |
| 51 | (±)-N-(5-Chloro-4-cyclobutoxy-2-fluorophenyl)-3-oxo-3,5,6,7,8,9-hexahydro- |
| | 2H-6,9-epiminocyclohepta[c]pyridazine-10-carboxamide |
| 52 | (±)-N-(5-Chloro-2,4-difluorophenyl)-3-oxo-3,5,6,7,8,9-hexahydro-2H-6,9- |
| | epiminocyclohepta[c]pyridazine-10-carboxamide |
| 53 | (±)-N-(5-Chloro-4-cyano-2-fluorophenyl)-3-oxo-3,5,6,7,8,9-hexahydro-2H- |
| | 6,9-epiminocyclohepta[c]pyridazine-10-carboxamide |
| 54 | (5R,8S)-N-(4,5-Dichloro-2-fluorophenyl)-2-methoxy-6,7,8,9-tetrahydro-5H- |
| | 5,8-epiminocyclohepta[b]pyridine-10-carboxamide |
| 55 | (5R,8S)-1-Fluoro-N-(4-(thiophen-3-yl)phenyl)-6,7,8,9-tetrahydro-5H-5,8- |
| | epiminocyclohepta[c]pyridine-10-carboxamide |
| 56 | (5R,8S)-1-Fluoro-N-(4-(2-methyloxazol-5-yl)phenyl)-6,7,8,9-tetrahydro-5H- |
| | 5,8-epiminocyclohepta[c]pyridine-10-carboxamide |
| 57 | (5R,8S)-1-Fluoro-N-(4-(tetrahydro-2H-pyran-4-yl)phenyl)-6,7,8,9-tetrahydro- |
| | 5H-5,8-epiminocyclohepta[c]pyridine-10-carboxamide |
| 58 | (5R,8S)-1-Fluoro-N-(4-(oxetan-3-yl)phenyl)-6,7,8,9-tetrahydro-5H-5,8- |
| | epiminocyclohepta[c]pyridine-10-carboxamide |
| 59 | (5R,8S)-N-(3-Chloro-4-cyclopropylphenyl)-1-fluoro-6,7,8,9-tetrahydro-5H- |
| | 5,8-epiminocyclohepta[c]pyridine-10-carboxamide |
| 60 | (5R,8S)-N-(3-Chloro-4-(2-hydroxypropan-2-yl)phenyl)-1-fluoro-6,7,8,9- |
| | tetrahydro-5H-5,8-epiminocyclohepta[c]pyridine-10-carboxamide |
| 61 | (5R,8S)-1-Fluoro-N-(4-(3-methyloxetan-3-yl)phenyl)-6,7,8,9-tetrahydro-5H- |
| | 5,8-epiminocyclohepta[c]pyridine-10-carboxamide |
| 62 | (5R,8S)-N-(4-(8-Oxa-3-azabicyclo[3.2.1]octan-3-yl)phenyl)-1-fluoro-6,7,8,9- |
| | tetrahydro-5H-5,8-epiminocyclohepta[c]pyridine-10-carboxamide |
| 63 | (5R,8S)-N-(5-Chloro-4-cyclopropyl-2-fluorophenyl)-1-fluoro-6,7,8,9- |
| | tetrahydro-5H-5,8-epiminocyclohepta[c]pyridine-10-carboxamide |
| 64 | (5R,8S)-N-(5-Chloro-2-fluoro-4-(5-methyl-1,3,4-thiadiazol-2-yl)phenyl)-1- |
| | fluoro-6,7,8,9-tetrahydro-5H-5,8-epiminocyclohepta[c]pyridine-10- |
| | carboxamide |
| 65 | (5R,8S)-N-(5-Chloro-2-fluoro-4-(5-(trifluoromethyl)-1,3,4-thiadiazol-2- |
| | yl)phenyl)-1-fluoro-6,7,8,9-tetrahydro-5H-5,8-epiminocyclohepta[c]pyridine- |
| | 10-carboxamide |
| 66 | (5R,8S)-1-Fluoro-N-(4-(5-(trifluoromethyl)-1,3,4-thiadiazol-2-yl)phenyl)- |
| | 6,7,8,9-tetrahydro-5H-5,8-epiminocyclohepta[c]pyridine-10-carboxamide |
| 67 | (5R,8S)-N-(5-Chloro-4-cyclobutoxy-2-fluorophenyl)-1-fluoro-6,7,8,9- |
| | tetrahydro-5H-5,8-epiminocyclohepta[c]pyridine-10-carboxamide |
| 68 | (5R,8S)-N-(5-Chloro-2-fluoro-4-(1-hydroxycyclopropyl)phenyl)-1-fluoro- |
| | 6,7,8,9-tetrahydro-5H-5,8-epiminocyclohepta[c]pyridine-10-carboxamide |
| 69 | (5R,8S)-N-(3-Chloro-4-cyclobutoxyphenyl)-1-fluoro-6,7,8,9-tetrahydro-5H- |
| | 5,8-epiminocyclohepta[c]pyridine-10-carboxamide |
| 70 | (5R,8S)-N-(3-Chloro-4-isopropoxyphenyl)-1-fluoro-6,7,8,9-tetrahydro-5H-5,8- |
| | epiminocyclohepta[c]pyridine-10-carboxamide |
| 71 | (5R,8S)-N-(3-Chloro-4-(oxetan-3-yloxy)phenyl)-1-fluoro-6,7,8,9-tetrahydro- |
| | 5H-5,8-epiminocyclohepta[c]pyridine-10-carboxamide |
| 72 | (5R,8S)-N-(3-Chloro-4-((1s,3S)-3-methoxycyclobutoxy)phenyl)-1-fluoro- |
| | 6,7,8,9-tetrahydro-5H-5,8-epiminocyclohepta[c]pyridine-10-carboxamide |
| 73 | (5R,8S)-N-(3-Chloro-4-((1r,3R)-3-methoxycyclobutoxy)phenyl)-1-fluoro- |
| | 6,7,8,9-tetrahydro-5H-5,8-epiminocyclohepta[c]pyridine-10-carboxamide |
| 74 | (±)-N-(5-Chloro-2-fluoro-4-iodophenyl)-3-oxo-3,5,6,7,8,9-hexahydro-2H-6,9- |
| | epiminocyclohepta[c]pyridazine-10-carboxamide |
| 75 | (±)-N-(3,4-Dichlorophenyl)-3-oxo-3,5,6,7,8,9-hexahydro-2H-6,9- |
| | epiminocyclohepta[c]pyridazine-10-carboxamide |
| 76 | (±)-N-(3-Chloro-4-(trifluoromethyl)phenyl)-3-oxo-3,5,6,7,8,9-hexahydro-2H- |
| | 6,9-epiminocyclohepta[c]pyridazine-10-carboxamide |
| 77 | (5R,8S)-N-(5-Chloro-2-fluoro-4-iodophenyl)-1-fluoro-6,7,8,9-tetrahydro-5H- |
| | 5,8-epiminocyclohepta[c]pyridine-10-carboxamide |
| 78 | (5R,8S)-N-(4-Chloro-2-fluoro-5-iodophenyl)-1-fluoro-6,7,8,9-tetrahydro-5H- |
| | 5,8-epiminocyclohepta[c]pyridine-10-carboxamide |
| 79 | (6S,9R)-N-(5-Chloro-2-fluoro-4-iodophenyl)-3-oxo-3,5,6,7,8,9-hexahydro-2H- |
| | 6,9-epiminocyclohepta[c]pyridine-10-carboxamide |
| 80 | (6S,9R)-N-(4-Chloro-2-fluoro-5-iodophenyl)-3-oxo-3,5,6,7,8,9-hexahydro-2H- |
| | 6,9-epiminocyclohepta[c]pyridine-10-carboxamide |
| 81 | (6S,9R)-N-(5-Chloro-4-cyclobutoxy-2-fluorophenyl)-3-oxo-3,5,6,7,8,9- |
| | hexahydro-2H-6,9-epiminocyclohepta[c]pyridine-10-carboxamide |
| 82 | (6S,9R)-N-(5-Chloro-4-cyclopropyl-2-fluorophenyl)-3-oxo-3,5,6,7,8,9- |
| | hexahydro-2H-6,9-epiminocyclohepta[c]pyridine-10-carboxamide |
| 83 | (±)-N-(4,5-Dichloro-2-fluorophenyl)-2-methyl-3-oxo-3,5,6,7,8,9-hexahydro- |
| | 2H-6,9-epiminocyclohepta[c]pyridazine-10-carboxamide |
| 84 | (±)-N-(4,5-Dichloro-2-fluorophenyl)-6,7,8,9-tetrahydro-5H-5,8- |
| | epiminocyclohepta[c]pyridazine-10-carboxamide |
| 85 | (5R,8S)-N-(5-Chloro-2-fluoro-4-(trifluoromethyl)phenyl)-6,7,8,9-tetrahydro- |
| | 5H-5,8-epiminocyclohepta[c]pyridazine-10-carboxamide |
| 87 | (±)-N-(4,5-Dichloro-2-fluorophenyl)-4-fluoro-6,7,8,9-tetrahydro-5H-6,9- |
| | epiminocyclohepta[d]pyrimidine-10-carboxamide |
| 88 | (5R,8S)-N-(4,5-Dichloro-2-fluorophenyl)-1-methoxy-6,7,8,9-tetrahydro-5H- |
| | 5,8-epiminocyclohepta[c]pyridine-10-carboxamide |
| 89 | (5R,8S)-N-(4,5-Dichloro-2-fluorophenyl)-1-(difluoromethyl)-6,7,8,9- |
| | tetrahydro-5H-5,8-epiminocyclohepta[c]pyridine-10-carboxamide |
| 90 | (6S,9R)-N-(4,5-Dichloro-2-fluorophenyl)-6,7,8,9-tetrahydro-5H-6,9- |
| | epiminocyclohepta[b]pyridine-10-carboxamide |
| 91 | (6S,9R)-N-(4,5-dichloro-2-fluorophenyl)-4-fluoro-6,7,8,9-tetrahydro-5H-6,9- |
| | epiminocyclohepta[b]pyridine-10-carboxamide |
| 92 | (5R,8S)-1-Cyano-N-(4,5-dichloro-2-fluorophenyl)-6,7,8,9-tetrahydro-5H-5,8- |
| | epiminocyclohepta[c]pyridine-10-carboxamide |
| 93 | (5R,8S)-N-(4,5-dichloro-2-fluorophenyl)-1-(difluoromethoxy)-6,7,8,9- |
| | tetrahydro-5H-5,8-epiminocyclohepta[c]pyridine-10-carboxamide |
| 95 | (6R,9S)-N-(4,5-Dichloro-2-fluorophenyl)-3-fluoro-6,7,8,9-tetrahydro-5H-6,9- |
| | epiminocyclohepta[b]pyridine-10-carboxamide |
| 96 | (5R,8S)-N-(4,5-Dichloro-2-fluorophenyl)-1-fluoro-4-methoxy-6,7,8,9- |
| | tetrahydro-5H-5,8-epiminocyclohepta[c]pyridine-10-carboxamide |
| 97 | (5R,8S)-N-(5-Chloro-2-fluoro-4-(trifluoromethyl)phenyl)-1-fluoro-4-methoxy- |
| | 6,7,8,9-tetrahydro-5H-5,8-epiminocyclohepta[c]pyridine-10-carboxamide |
| 98 | (6S,9R)-N-(4,5-Dichloro-2-fluorophenyl)-3-fluoro-6,7,8,9-tetrahydro-5H-6,9- |
| | epiminocyclohepta[b]pyridine-10-carboxamide |
| 99 | (6S,9R)-4-Chloro-N-(4,5-dichloro-2-fluorophenyl)-1-fluoro-6,7,8,9-tetrahydro- |
| | 5H-6,9-epiminocyclohepta[c]pyridine-10-carboxamide |
| 100 | (5R,8S)-4-Chloro-N-(4,5-dichloro-2-fluorophenyl)-1-fluoro-6,7,8,9-tetrahydro- |
| | 5H-5,8-epiminocyclohepta[c]pyridine-10-carboxamide |
| 102 | (5R,8S)-4-(Benzyloxy)-N-(4,5-dichloro-2-fluorophenyl)-1-fluoro-6,7,8,9- |
| | tetrahydro-5H-5,8-epiminocyclohepta[c]pyridine-10-carboxamide |
| 103 | (5R,8S)-N-(4,5-Dichloro-2-fluorophenyl)-1-fluoro-5-methyl-6,7,8,9-tetrahydro- |
| | 5H-5,8-epiminocyclohepta[c]pyridine-10-carboxamide |
| 104 | (5R,8S)-N-(4,5-Dichloro-2-fluorophenyl)-1-fluoro-4-hydroxy-6,7,8,9- |
| | tetrahydro-5H-5,8-epiminocyclohepta[c]pyridine-10-carboxamide |
| 105 | (5R,8S)-N-(4,5-Dichloro-2-fluorophenyl)-1-fluoro-4-(3-methoxypropoxy)- |
| | 6,7,8,9-tetrahydro-5H-5,8-epiminocyclohepta[c]pyridine-10-carboxamide |
| 106 | (6S,9R)-N-(4,5-Dichloro-2-fluorophenyl)-1-fluoro-5-methylene-6,7,8,9- |
| | tetrahydro-5H-6,9-epiminocyclohepta[c]pyridine-10-carboxamide |
| 107 | (5R,8S)-N-(4,5-Dichloro-2-fluorophenyl)-1-fluoro-4-(3-hydroxyprop-1-yn-1- |
| | yl)-6,7,8,9-tetrahydro-5H-5,8-epiminocyclohepta[c]pyridine-10-carboxamide |
| 108 | (6S,9R)-N-(4-(Benzo[c][1,2,5]oxadiazol-5-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 109 | (6S,9R)-N-(5-Chloro-2-fluoro-4-(6-fluoropyridin-3-yl)phenyl)-3-oxo- |
| | 3,5,6,7,8,9-hexahydro-2H-6,9-epiminocyclohepta[c]pyridazine-10- |
| | carboxamide |
| 110 | (6S,9R)-3-Oxo-N-(5-(trifluoromethyl)isoxazol-3-yl)-3,5,6,7,8,9-hexahydro-2H- |
| | 6,9-epiminocyclohepta[c]pyridazine-10-carboxamide |
| 111 | (6S,9R)-N-(5-Chloro-4-cyano-2-fluorophenyl)-3-oxo-3,5,6,7,8,9-hexahydro- |
| | 2H-6,9-epiminocyclohepta[c]pyridazine-10-carboxamide |
| 112 | (6S,9R)-N-(4-Chloro-5-cyano-2-fluorophenyl)-3-oxo-3,5,6,7,8,9-hexahydro- |
| | 2H-6,9-epiminocyclohepta[c]pyridazine-10-carboxamide |
| 113 | (6S,9R)-N-(2-Chloro-5-fluoro-[1,1′-biphenyl]-4-yl)-3-oxo-3,5,6,7,8,9- |
| | hexahydro-2H-6,9-epiminocyclohepta[c]pyridazine-10-carboxamide |
| 114 | (6S,9R)-N-(5-Chloro-2-fluoro-4-(5-methoxypyridin-3-yl)phenyl)-3-oxo- |
| | 3,5,6,7,8,9-hexahydro-2H-6,9-epiminocyclohepta[c]pyridazine-10- |
| | carboxamide |
| 115 | (6S,9R)-N-(5-Chloro-2-fluoro-4-(2-fluoropyridin-3-yl)phenyl)-3-oxo- |
| | 3,5,6,7,8,9-hexahydro-2H-6,9-epiminocyclohepta[c]pyridazine-10- |
| | carboxamide |
| 116 | (6S,9R)-N-(2-Fluoro-4,5-bis(2-fluoropyridin-3-yl)phenyl)-3-oxo-3,5,6,7,8,9- |
| | hexahydro-2H-6,9-epiminocyclohepta[c]pyridazine-10-carboxamide |
| 117 | (6S,9R)-N-(4-(6-Acetamidopyridin-3-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 118 | (6S,9R)-N-(4-(6-aminopyridin-3-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 119 | (6S,9R)-N-(5-Chloro-2-fluoro-4-(1H-pyrazol-4-yl)phenyl)-3-oxo-3,5,6,7,8,9- |
| | hexahydro-2H-6,9-epiminocyclohepta[c]pyridazine-10-carboxamide |
| 120 | (6S,9R)-N-(5-Chloro-2-fluoro-4-(1-methyl-1H-indazol-4-yl)phenyl)-3-oxo- |
| | 3,5,6,7,8,9-hexahydro-2H-6,9-epiminocyclohepta[c]pyridazine-10- |
| | carboxamide |
| 121 | (6S,9R)-N-(5-Chloro-2-fluoro-4-(2-((2-methoxyethyl)amino)pyrimidin-5- |
| | yl)phenyl)-3-oxo-3,5,6,7,8,9-hexahydro-2H-6,9- |
| | epiminocyclohepta[c]pyridazine-10-carboxamide |
| 122 | (6S,9R)-N-(5-Chloro-2-fluoro-4-(5-(methylsulfonyl)pyridin-3-yl)phenyl)-3-oxo- |
| | 3,5,6,7,8,9-hexahydro-2H-6,9-epiminocyclohepta[c]pyridazine-10- |
| | carboxamide |
| 123 | (6S,9R)-N-(5-Chloro-2-fluoro-4-(6-methylpyridin-3-yl)phenyl)-3-oxo- |
| | 3,5,6,7,8,9-hexahydro-2H-6,9-epiminocyclohepta[c]pyridazine-10- |
| | carboxamide |
| 124 | (6S,9R)-N-(5-Chloro-4-(6-cyanopyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 125 | (6S,9R)-N-(5-Chloro-2-fluoro-4-(3-methylpyridin-4-yl)phenyl)-3-oxo- |
| | 3,5,6,7,8,9-hexahydro-2H-6,9-epiminocyclohepta[c]pyridazine-10- |
| | carboxamide |
| 126 | (6S,9R)-N-(5-Chloro-2-fluoro-4-(5-fluoropyridin-3-yl)phenyl)-3-oxo- |
| | 3,5,6,7,8,9-hexahydro-2H-6,9-epiminocyclohepta[c]pyridazine-10- |
| | carboxamide |
| 127 | (6S,9R)-N-(5-Chloro-4-(5-cyanopyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 128 | (6S,9R)-N-(5-Chloro-2-fluoro-4-(5-(hydroxymethyl)pyridin-3-yl)phenyl)-3-oxo- |
| | 3,5,6,7,8,9-hexahydro-2H-6,9-epiminocyclohepta[c]pyridazine-10- |
| | carboxamide |
| 129 | (6S,9R)-N-(4′-Carbamoyl-2-chloro-5-fluoro-[1,1′-biphenyl]-4-yl)-3-oxo- |
| | 3,5,6,7,8,9-hexahydro-2H-6,9-epiminocyclohepta[c]pyridazine-10- |
| | carboxamide |
| 130 | (6S,9R)-N-(2-Chloro-5-fluoro-4′-(methylcarbamoyl)-[1,1′-biphenyl]-4-yl)-3-oxo- |
| | 3,5,6,7,8,9-hexahydro-2H-6,9-epiminocyclohepta[c]pyridazine-10- |
| | carboxamide |
| 131 | (6S,9R)-N-(2-Chloro-5-fluoro-4′-(methylsulfonyl)-[1,1′-biphenyl]-4-yl)-3-oxo- |
| | 3,5,6,7,8,9-hexahydro-2H-6,9-epiminocyclohepta[c]pyridazine-10- |
| | carboxamide |
| 132 | (6S,9R)-N-(2-Chloro-3′,5-difluoro-4′-(methylsulfonyl)-[1,1′-biphenyl]-4-yl)-3- |
| | oxo-3,5,6,7,8,9-hexahydro-2H-6,9-epiminocyclohepta[c]pyridazine-10- |
| | carboxamide |
| 133 | (6S,9R)-N-(2-Chloro-3′-((dimethylamino)methyl)-5-fluoro-[1,1′-biphenyl]-4-yl)- |
| | 3-oxo-3,5,6,7,8,9-hexahydro-2H-6,9-epiminocyclohepta[c]pyridazine-10- |
| | carboxamide |
| 134 | (6S,9R)-N-(5-Chloro-2-fluoro-4-(2-methylpyridin-4-yl)phenyl)-3-oxo- |
| | 3,5,6,7,8,9-hexahydro-2H-6,9-epiminocyclohepta[c]pyridazine-10- |
| | carboxamide |
| 135 | (6S,9R)-N-(2-Chloro-5-fluoro-3′-(pyrrolidin-1-ylmethyl)-[1,1′-biphenyl]-4-yl)-3- |
| | oxo-3,5,6,7,8,9-hexahydro-2H-6,9-epiminocyclohepta[c]pyridazine-10- |
| | carboxamide |
| 136 | (6S,9R)-N-(5-Chloro-2-fluoro-4-(pyridin-3-yl)phenyl)-3-oxo-3,5,6,7,8,9- |
| | hexahydro-2H-6,9-epiminocyclohepta[c]pyridazine-10-carboxamide |
| 137 | (6S,9R)-N-(5-Chloro-2-fluoro-4-(pyrimidin-5-yl)phenyl)-3-oxo-3,5,6,7,8,9- |
| | hexahydro-2H-6,9-epiminocyclohepta[c]pyridazine-10-carboxamide |
| 138 | (6S,9R)-N-(5-Chloro-2-fluoro-4-(3-fluoropyridin-4-yl)phenyl)-3-oxo- |
| | 3,5,6,7,8,9-hexahydro-2H-6,9-epiminocyclohepta[c]pyridazine-10- |
| | carboxamide |
| 139 | (6S,9R)-N-(5-Chloro-2-fluoro-4-(2-methyl-2H-1,2,3-triazol-4-yl)phenyl)-3-oxo- |
| | 3,5,6,7,8,9-hexahydro-2H-6,9-epiminocyclohepta[c]pyridazine-10- |
| | carboxamide |
| 140 | (6S,9R)-N-(4-(2H-benzo[d][1,2,3]triazol-5-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 141 | (6S,9R)-N-(5-Chloro-2-fluoro-4-(1-methyl-1H-benzo[d][1,2,3]triazol-6- |
| | yl)phenyl)-3-oxo-3,5,6,7,8,9-hexahydro-2H-6,9- |
| | epiminocyclohepta[c]pyridazine-10-carboxamide |
| 142 | (6S,9R)-N-(5-Chloro-2-fluoro-4-(1-oxoisoindolin-5-yl)phenyl)-3-oxo- |
| | 3,5,6,7,8,9-hexahydro-2H-6,9-epiminocyclohepta[c]pyridazine-10- |
| | carboxamide |
| 143 | (6S,9R)-N-(5-Chloro-2-fluoro-4-(1-(tetrahydro-2H-pyran-2-yl)-1H-imidazol-5- |
| | yl)phenyl)-3-oxo-3,5,6,7,8,9-hexahydro-2H-6,9- |
| | epiminocyclohepta[c]pyridazine-10-carboxamide |
| 144 | (6S,9R)-N-(5-Chloro-4-(1-(cyclopropylmethyl)-1H-pyrazol-4-yl)-2- |
| | fluorophenyl)-3-oxo-3,5,6,7,8,9-hexahydro-2H-6,9- |
| | epiminocyclohepta[c]pyridazine-10-carboxamide |
| 145 | (6S,9R)-N-(5-Chloro-2-fluoro-4-(1-(pyridin-4-yl)-1H-pyrazol-4-yl)phenyl)-3- |
| | oxo-3,5,6,7,8,9-hexahydro-2H-6,9-epiminocyclohepta[c]pyridazine-10- |
| | carboxamide |
| 146 | (6S,9R)-N-(5-Chloro-2-fluoro-4-(3-oxo-3,4-dihydro-2H-benzo[b][1,4]oxazin-6- |
| | yl)phenyl)-3-oxo-3,5,6,7,8,9-hexahydro-2H-6,9- |
| | epiminocyclohepta[c]pyridazine-10-carboxamide |
| 147 | (6S,9R)-N-(5-Chloro-2-fluoro-4-(4-methyl-3-oxo-3,4-dihydro-2H- |
| | benzo[b][1,4]oxazin-6-yl)phenyl)-3-oxo-3,5,6,7,8,9-hexahydro-2H-6,9- |
| | epiminocyclohepta[c]pyridazine-10-carboxamide |
| 148 | (6S,9R)-N-(5-Chloro-2-fluoro-4-(1H-pyrrolo[2,3-b]89yridine-4-yl)phenyl)-3- |
| | oxo-3,5,6,7,8,9-hexahydro-2H-6,9-epiminocyclohepta[c]pyridazine-10- |
| | carboxamide |
| 149 | (6S,9R)-N-(5-Chloro-4-(3,5-dimethyl-1H-pyrazol-4-yl)-2-fluorophenyl)-3-oxo- |
| | 3,5,6,7,8,9-hexahydro-2H-6,9-epiminocyclohepta[c]pyridazine-10- |
| | carboxamide |
| 150 | (6S,9R)-N-(5-Chloro-2-fluoro-4-(1,3,5-trimethyl-1H-pyrazol-4-yl)phenyl)-3- |
| | oxo-3,5,6,7,8,9-hexahydro-2H-6,9-epiminocyclohepta[c]pyridazine-10- |
| | carboxamide |
| 151 | (6S,9R)-N-(5-chloro-2-fluoro-4-(1-oxo-1,2,3,4-tetrahydroisoquinolin-6- |
| | yl)phenyl)-3-oxo-3,5,6,7,8,9-hexahydro-2H-6,9- |
| | epiminocyclohepta[c]pyridazine-10-carboxamide |
| 152 | (6S,9R)-N-(5-Chloro-2-fluoro-4-(1-methyl-1H-indazol-6-yl)phenyl)-3-oxo- |
| | 3,5,6,7,8,9-hexahydro-2H-6,9-epiminocyclohepta[c]pyridazine-10- |
| | carboxamide |
| 153 | (6S,9R)-N-(5-Chloro-2-fluoro-4-(2-oxoindolin-6-yl)phenyl)-3-oxo-3,5,6,7,8,9- |
| | hexahydro-2H-6,9-epiminocyclohepta[c]pyridazine-10-carboxamide |
| 154 | (±)-N-(4,5-Dichloro-2-fluorophenyl)-1-fluoro-9-methylene-6,7,8,9-tetrahydro- |
| | 5H-5,8-epiminocyclohepta[c]pyridine-10-carboxamide |
| 155 | (±)-N-(4,5-Dichloro-2-fluorophenyl)-1-fluoro-9-oxo-6,7,8,9-tetrahydro-5H-5,8- |
| | epiminocyclohepta[c]pyridine-10-carboxamide |
| 156 | (±)-(9S)-N-(5-chloro-2-fluoro-4-(trifluoromethyl)phenyl)-1-fluoro-9-hydroxy- |
| | 6,7,8,9-tetrahydro-5H-5,8-epiminocyclohepta[c]pyridine-10-carboxamide |
| 157 | (±)-(9R)-N-(5-chloro-2-fluoro-4-(trifluoromethyl)phenyl)-1,9-difluoro-6,7,8,9- |
| | tetrahydro-5H-5,8-epiminocyclohepta[c]pyridine-10-carboxamide |
| 158 | (±)-N-(4,5-dichloro-2-fluorophenyl)-2-oxo-2,5,6,7,8,9-hexahydro-1H-5,8- |
| | epiminocyclohepta[b]pyrazine-10-carboxamide |
| 159 | (5R,8S)-N-(4,5-dichloro-2-fluorophenyl)-1-fluoro-4-(methylamino)-6,7,8,9- |
| | tetrahydro-5H-5,8-epiminocyclohepta[c]pyridine-10-carboxamide |
| 160 | (5R,8S)-4-Cyano-N-(4,5-dichloro-2-fluorophenyl)-1-fluoro-6,7,8,9-tetrahydro- |
| | 5H-5,8-epiminocyclohepta[c]pyridine-10-carboxamide |
| 161 | (5R,8S)-N-(5-Chloro-2-fluoro-4-(trifluoromethyl)phenyl)-1-fluoro-4-(3- |
| | (methylamino)propoxy)-6,7,8,9-tetrahydro-5H-5,8- |
| | epiminocyclohepta[c]pyridine-10-carboxamide |
| 162 | (5R,8S)-N-(4-(Benzo[c][1,2,5]oxadiazol-5-yl)-5-chloro-2-fluorophenyl)-1- |
| | fluoro-6,7,8,9-tetrahydro-5H-5,8-epiminocyclohepta[c]pyridine-10- |
| | carboxamide |
| 163 | (6S,9R)-N-(5-Chloro-2-fluoro-4-(1-methyl-2-oxoindolin-6-yl)phenyl)-3-oxo- |
| | 3,5,6,7,8,9-hexahydro-2H-6,9-epiminocyclohepta[c]pyridazine-10- |
| | carboxamide |
| 164 | (6S,9R)-N-(5-Chloro-2-fluoro-4-(1-methyl-1H-indazol-5-yl)phenyl)-3-oxo- |
| | 3,5,6,7,8,9-hexahydro-2H-6,9-epiminocyclohepta[c]pyridazine-10- |
| | carboxamide |
| 165 | (6S,9R)-N-(4-([1,2,5]Oxadiazolo[3,4-b]pyridine-6-yl)-5-chloro-2-fluorophenyl)- |
| | 3-oxo-3,5,6,7,8,9-hexahydro-2H-6,9-epiminocyclohepta[c]pyridazine-10- |
| | carboxamide |
| 166 | (6S,9R)-3-Oxo-N-(5-phenylisoxazol-3-yl)-3,5,6,7,8,9-hexahydro-2H-6,9- |
| | epiminocyclohepta[c]pyridazine-10-carboxamide |
| 167 | (6S,9R)-N-(4-(Benzo[c][1,2,5]oxadiazol-5-yl)-5-chloro-2-fluorophenyl)-3-oxo- |
| | 3,5,6,7,8,9-hexahydro-2H-6,9-epiminocyclohepta[c]pyridine-10-carboxamide |
| 168 | (6S,9R)-N-(4-(Benzo[c][1,2,5]thiadiazol-5-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 169 | (5R,8S)-N-(5-Chloro-2-fluoro-4-(6-fluoropyridin-3-yl)phenyl)-1-fluoro-6,7,8,9- |
| | tetrahydro-5H-5,8-epiminocyclohepta[c]pyridine-10-carboxamide |
| 170 | (6S,9R)-N-(5-Chloro-2-fluoro-4-(2-methylbenzo[d]oxazol-6-yl)phenyl)-3-oxo- |
| | 3,5,6,7,8,9-hexahydro-2H-6,9-epiminocyclohepta[c]pyridazine-10- |
| | carboxamide |
| 171 | (6S,9R)-N-(5-Chloro-2-fluoro-4-(6-methoxypyridin-3-yl)phenyl)-3-oxo- |
| | 3,5,6,7,8,9-hexahydro-2H-6,9-epiminocyclohepta[c]pyridazine-10- |
| | carboxamide |
| 172 | (6S,9R)-N-(5-Chloro-2-fluoro-4-(6-fluoropyridin-3-yl)phenyl)-3-oxo- |
| | 3,5,6,7,8,9-hexahydro-2H-6,9-epiminocyclohepta[c]pyridine-10-carboxamide |
| 173 | (6S,9R)-N-(5-chloro-2-fluoro-4-(pyridin-4-yl)phenyl)-3-oxo-3,5,6,7,8,9- |
| | hexahydro-2H-6,9-epiminocyclohepta[c]pyridazine-10-carboxamide |
| 174 | (6S,9R)-N-(5-Chloro-2-fluoro-4-(pyridazin-4-yl)phenyl)-3-oxo-3,5,6,7,8,9- |
| | hexahydro-2H-6,9-epiminocyclohepta[c]pyridazine-10-carboxamide |
| 175 | (6S,9R)-N-(5-Chloro-2-fluoro-4-(2-methyl-1-oxo-1,2,3,4-tetrahydroisoquinolin- |
| | 6-yl)phenyl)-3-oxo-3,5,6,7,8,9-hexahydro-2H-6,9- |
| | epiminocyclohepta[c]pyridazine-10-carboxamide |
| 176 | (6S,9R)-N-(4-(benzo[c][1,2,5]thiadiazol-5-yl)-5-chloro-2-fluorophenyl)-3-oxo- |
| | 3,5,6,7,8,9-hexahydro-2H-6,9-epiminocyclohepta[c]pyridine-10-carboxamide |
| 177 | (5R,8S)-N-(4-(benzo[c][1,2,5]thiadiazol-5-yl)-5-chloro-2-fluorophenyl)-1- |
| | fluoro-6,7,8,9-tetrahydro-5H-5,8-epiminocyclohepta[c]pyridine-10- |
| | carboxamide |
| 178 | (6S,9R)-N-(5-Chloro-2-fluoro-4-(2-fluoropyrimidin-5-yl)phenyl)-3-oxo- |
| | 3,5,6,7,8,9-hexahydro-2H-6,9-epiminocyclohepta[c]pyridazine-10- |
| | carboxamide |
| 179 | (6S,9R)-N-(5-Chloro-4-(3-cyanopyridin-4-yl)-2-fluorophenyl)-3-oxo- |
| | 3,5,6,7,8,9-hexahydro-2H-6,9-epiminocyclohepta[c]pyridazine-10- |
| | carboxamide |
| 180 | (6S,9R)-N-(5-Chloro-2-fluoro-4-(6-(methylsulfonyl)pyridin-3-yl)phenyl)-3-oxo- |
| | 3,5,6,7,8,9-hexahydro-2H-6,9-epiminocyclohepta[c]pyridazine-10- |
| | carboxamide |
| 181 | (6S,9R)-N-(5-Chloro-2-fluoro-4-(oxazol-2-yl)phenyl)-3-oxo-3,5,6,7,8,9- |
| | hexahydro-2H-6,9-epiminocyclohepta[c]pyridazine-10-carboxamide |
| 182 | (6S,9R)-N-(5-Chloro-4-(2,5-difluoropyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 183 | (±)-N-(5-chloro-2-fluoro-4-(trifluoromethyl)phenyl)-1,9,9-trifluoro-6,7,8,9- |
| | tetrahydro-5H-5,8-epiminocyclohepta[c]pyridine-10-carboxamide |
| 184 | (6S,9R)-N-(5-Chloro-2-fluoro-4-(6-methoxypyridin-3-yl)phenyl)-3-oxo- |
| | 3,5,6,7,8,9-hexahydro-2H-6,9-epiminocyclohepta[c]pyridine-10-carboxamide |
| 185 | (5R,8S)-N-(5-Chloro-2-fluoro-4-(6-methoxypyridin-3-yl)phenyl)-1-fluoro- |
| | 6,7,8,9-tetrahydro-5H-5,8-epiminocyclohepta[c]pyridine-10-carboxamide |
| 186 | (6S,9R)-N-(benzo[c][1,2,5]thiadiazol-5-yl)-3-oxo-3,5,6,7,8,9-hexahydro-2H- |
| | 6,9-epiminocyclohepta[c]pyridazine-10-carboxamide |
| 187 | (6S,9R)-N-(Benzo[d]isoxazol-3-yl)-3-oxo-3,5,6,7,8,9-hexahydro-2H-6,9- |
| | epiminocyclohepta[c]pyridazine-10-carboxamide |
| 188 | Methyl 5-(2-chloro-5-fluoro-4-((6S,9R)-3-oxo-3,5,6,7,8,9-hexahydro-2H-6,9- |
| | epiminocyclohepta[c]pyridazine-10-carboxamido)phenyl)nicotinate |
| 189 | (6S,9R)-N-(4-([1,2,5]oxadiazolo[3,4-b]pyridin-6-yl)-5-chloro-2-fluorophenyl)-3- |
| | oxo-3,5,6,7,8,9-hexahydro-2H-6,9-epiminocyclohepta[c]pyridine-10- |
| | carboxamide |
| 190 | (5R,8S)-N-(4-([1,2,5]Oxadiazolo[3,4-b]pyridin-6-yl)-5-chloro-2-fluorophenyl)- |
| | 1-fluoro-6,7,8,9-tetrahydro-5H-5,8-epiminocyclohepta[c]pyridine-10- |
| | carboxamide |
| 191 | (6S,9R)-N-(5-Chloro-2-fluoro-4-(isothiazol-3-yl)phenyl)-3-oxo-3,5,6,7,8,9- |
| | hexahydro-2H-6,9-epiminocyclohepta[c]pyridazine-10-carboxamide |
| 192 | (6S,9R)-N-(5-Chloro-2-fluoro-4-(4-(methoxymethyl)pyridin-3-yl)phenyl)-3-oxo- |
| | 3,5,6,7,8,9-hexahydro-2H-6,9-epiminocyclohepta[c]pyridazine-10- |
| | carboxamide |
| 193 | (5R,8S,9R)-N-(5-Chloro-2-fluoro-4-(trifluoromethyl)phenyl)-1,9-difluoro- |
| | 6,7,8,9-tetrahydro-5H-5,8-epiminocyclohepta[c]pyridine-10-carboxamide |
| 194 | (5S,8R)-N-(5-Chloro-2-fluoro-4-(trifluoromethyl)phenyl)-1-fluoro-6,7,8,9- |
| | tetrahydro-5H-5,8-epiminocyclohepta[c]pyridine-10-carboxamide |
| 195 | (6S,9R)-N-(5-Chloro-2-fluoro-4-(pyridin-2-yl)phenyl)-3-oxo-3,5,6,7,8,9- |
| | hexahydro-2H-6,9-epiminocyclohepta[c]pyridazine-10-carboxamide |
| 196 | (6S,9R)-N-(4-(Benzo[d]isoxazol-6-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 197 | (6S,9R)-N-(5-Chloro-2-fluoro-4-(imidazo[1,5-a]pyridin-6-yl)phenyl)-3-oxo- |
| | 3,5,6,7,8,9-hexahydro-2H-6,9-epiminocyclohepta[c]pyridazine-10- |
| | carboxamide |
| 198 | (6S,9R)-N-(4-(Benzo[c]isoxazol-6-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 199 | (6S,9R)-N-(5-Chloro-2-fluoro-4-(2-methoxypyridin-4-yl)phenyl)-3-oxo- |
| | 3,5,6,7,8,9-hexahydro-2H-6,9-epiminocyclohepta[c]pyridazine-10- |
| | carboxamide |
| 200 | (5R,8S)-N-(5-Chloro-2-fluoro-4-(5-fluoropyridin-3-yl)phenyl)-1-fluoro-6,7,8,9- |
| | tetrahydro-5H-5,8-epiminocyclohepta[c]pyridine-10-carboxamide |
| 201 | (6S,9R)-N-(5-Chloro-2-fluoro-4-(5-fluoropyridin-3-yl)phenyl)-3-oxo- |
| | 3,5,6,7,8,9-hexahydro-2H-6,9-epiminocyclohepta[c]pyridine-10-carboxamide |
| 202 | (6S,9R)-N-(5-Chloro-2-fluoro-4-(2-fluoro-6-methoxypyridin-3-yl)phenyl)-3-oxo- |
| | 3,5,6,7,8,9-hexahydro-2H-6,9-epiminocyclohepta[c]pyridazine-10- |
| | carboxamide |
| 203 | (6S,9R)-N-(2-Chloro-5-fluoro-3′-(methylsulfonamidomethyl)-[1,1′-biphenyl]-4- |
| | yl)-3-oxo-3,5,6,7,8,9-hexahydro-2H-6,9-epiminocyclohepta[c]pyridazine-10- |
| | carboxamide |
| 204 | (±)-(R)-N-(5-chloro-2-fluoro-4-(trifluoromethyl)phenyl)-1-fluoro-9-hydroxy- |
| | 6,7,8,9-tetrahydro-5H-5,8-epiminocyclohepta[c]pyridine-10-carboxamide |
| 205 | (5R,8S)-N-(5-Chloro-2-fluoro-4-(2-methylbenzo[d]oxazol-6-yl)phenyl)-1- |
| | fluoro-6,7,8,9-tetrahydro-5H-5,8-epiminocyclohepta[c]pyridine-10- |
| | carboxamide |
| 206 | (6S,9R)-N-(5-Chloro-2-fluoro-4-(2-methylbenzo[d]oxazol-6-yl)phenyl)-3-oxo- |
| | 3,5,6,7,8,9-hexahydro-2H-6,9-epiminocyclohepta[c]pyridine-10-carboxamide |
| 207 | (6S,9R)-N-(5-Chloro-2-fluoro-4-(5-fluoro-6-methoxypyridin-3-yl)phenyl)-3-oxo- |
| | 3,5,6,7,8,9-hexahydro-2H-6,9-epiminocyclohepta[c]pyridazine-10- |
| | carboxamide |
| 208 | (6S,9R)-N-(5-chloro-4-(6-ethoxypyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 209 | (6S,9R)-N-(5-Chloro-2-fluoro-4-(6-isopropoxypyridin-3-yl)phenyl)-3-oxo- |
| | 3,5,6,7,8,9-hexahydro-2H-6,9-epiminocyclohepta[c]pyridazine-10- |
| | carboxamide |
| 210 | (6S,9R)-N-(4-(Benzo[c][1,2,5]oxadiazol-4-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 211 | (6S,9R)-N-(5-Chloro-2-fluoro-4-(5-methoxypyridin-2-yl)phenyl)-3-oxo- |
| | 3,5,6,7,8,9-hexahydro-2H-6,9-epiminocyclohepta[c]pyridazine-10- |
| | carboxamide |
| 212 | (6S,9R)-N-(2-Fluoro-4-(6-fluoropyridin-3-yl)-5-(trifluoromethyl)phenyl)-3-oxo- |
| | 3,5,6,7,8,9-hexahydro-2H-6,9-epiminocyclohepta[c]pyridazine-10- |
| | carboxamide |
| 213 | (6S,9R)-N-(2-Fluoro-4-(6-methoxypyridin-3-yl)-5-(trifluoromethyl)phenyl)-3- |
| | oxo-3,5,6,7,8,9-hexahydro-2H-6,9-epiminocyclohepta[c]pyridazine-10- |
| | carboxamide |
| 214 | (5R,8S)-N-(5-Chloro-2-fluoro-4-(2-fluoro-6-methoxypyridin-3-yl)phenyl)-1- |
| | fluoro-6,7,8,9-tetrahydro-5H-5,8-epiminocyclohepta[c]pyridine-10- |
| | carboxamide |
| 215 | (6S,9R)-N-(5-Chloro-2-fluoro-4-(6-(2,2,2-trifluoroethoxy)pyridin-3-yl)phenyl)- |
| | 3-oxo-3,5,6,7,8,9-hexahydro-2H-6,9-epiminocyclohepta[c]pyridazine-10- |
| | carboxamide |
| 216 | (6S,9R)-N-(5-Chloro-4-(6-cyclobutoxypyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 217 | (6S,9R)-N-(5-Chloro-2-fluoro-4-(5-fluoro-6-methoxypyridin-3-yl)phenyl)-3-oxo- |
| | 3,5,6,7,8,9-hexahydro-2H-6,9-epiminocyclohepta[c]pyridine-10-carboxamide |
| 218 | (6S,9R)-N-(5-chloro-2-fluoro-4-(2-isopropoxypyridin-4-yl)phenyl)-3-oxo- |
| | 3,5,6,7,8,9-hexahydro-2H-6,9-epiminocyclohepta[c]pyridazine-10- |
| | carboxamide |
| 219 | (5R,8S)-N-(5-Chloro-2-fluoro-4-(5-fluoro-6-methoxypyridin-3-yl)phenyl)-1- |
| | fluoro-6,7,8,9-tetrahydro-5H-5,8-epiminocyclohepta[c]pyridine-10- |
| | carboxamide |
| 220 | (5R,8S)-N-(5-Chloro-2-fluoro-4-(6-(2-(piperidin-1-yl)ethoxy)pyridin-3- |
| | yl)phenyl)-1-fluoro-6,7,8,9-tetrahydro-5H-5,8-epiminocyclohepta[c]pyridine- |
| | 10-carboxamide |
| 221 | (6S,9R)-N-(5-Chloro-4-(6-cyclopropoxypyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 222 | (6S,9R)-N-(5-Chloro-4-(6-cyclopropoxypyridin-3-yl)-2-fluorophenyl)-3-oxo- |
| | 3,5,6,7,8,9-hexahydro-2H-6,9-epiminocyclohepta[c]pyridine-10-carboxamide |
| 223 | (5R,8S)-N-(2,5-Difluoro-4-(6-(trifluoromethyl)pyridin-3-yl)phenyl)-1-fluoro- |
| | 6,7,8,9-tetrahydro-5H-5,8-epiminocyclohepta[c]pyridine-10-carboxamide |
| 224 | (6S,9R)-N-(2-(Benzo[c][1,2,5]oxadiazol-5-yloxy)-5-chloro-4-fluorophenyl)-3- |
| | oxo-3,5,6,7,8,9-hexahydro-2H-6,9-epiminocyclohepta[c]pyridazine-10- |
| | carboxamide |
| 225 | (6S,9R)-N-(5-Chloro-2-fluoro-4-((6-fluoropyridin-3-yl)oxy)phenyl)-3-oxo- |
| | 3,5,6,7,8,9-hexahydro-2H-6,9-epiminocyclohepta[c]pyridazine-10- |
| | carboxamide |
| 226 | (±, 9-endo)-N-(5-chloro-2-fluoro-4-(trifluoromethyl)phenyl)-1,9-difluoro- |
| | 6,7,8,9-tetrahydro-5H-5,8-epiminocyclohepta[c]pyridine-10-carboxamide |
| 227 | (5R,8S)-N-(5-Chloro-2-fluoro-4-(trifluoromethyl)phenyl)-1,9,9-trifluoro-6,7,8,9- |
| | tetrahydro-5H-5,8-epiminocyclohepta[c]pyridine-10-carboxamide |
| 228 | (5S,8R)-N-(5-Chloro-2-fluoro-4-(trifluoromethyl)phenyl)-1,9,9-trifluoro-6,7,8,9- |
| | tetrahydro-5H-5,8-epiminocyclohepta[c]pyridine-10-carboxamide |
| 229 | (6S,9R)-N-(4-Chloro-5′-fluoro-[3,3′-bipyridin]-6-yl)-3-oxo-3,5,6,7,8,9- |
| | hexahydro-2H-6,9-epiminocyclohepta[c]pyridazine-10-carboxamide |
| 230 | (6S,9R)-N-(4-Chloro-6′-fluoro-[3,3′-bipyridin]-6-yl)-3-oxo-3,5,6,7,8,9- |
| | hexahydro-2H-6,9-epiminocyclohepta[c]pyridazine-10-carboxamide |
| 231 | (6S,9R)-N-(5-Chloro-2-fluoro-4-(phenylcarbamoyl)phenyl)-3-oxo-3,5,6,7,8,9- |
| | hexahydro-2H-6,9-epiminocyclohepta[c]pyridazine-10-carboxamide |
| 232 | (6S,9R)-N-(5-Chloro-2-fluoro-4-(2-methylbenzo[d]oxazol-5-yl)phenyl)-3-oxo- |
| | 3,5,6,7,8,9-hexahydro-2H-6,9-epiminocyclohepta[c]pyridine-10-carboxamide |
| 233 | (5R,8S)-N-(5-Chloro-2-fluoro-4-(2-methylbenzo[d]oxazol-5-yl)phenyl)-1- |
| | fluoro-6,7,8,9-tetrahydro-5H-5,8-epiminocyclohepta[c]pyridine-10- |
| | carboxamide |
| 234 | (6S,9R)-N-(5-Chloro-2-fluoro-4-(2-methylbenzo[d]oxazol-5-yl)phenyl)-3-oxo- |
| | 3,5,6,7,8,9-hexahydro-2H-6,9-epiminocyclohepta[c]pyridazine-10- |
| | carboxamide |
| 235 | (6S,9R)-N-(5-Chloro-2-fluoro-4-(phenoxymethyl)phenyl)-3-oxo-3,5,6,7,8,9- |
| | hexahydro-2H-6,9-epiminocyclohepta[c]pyridazine-10-carboxamide |
| 236 | (6S,9R)-N-(4-Benzamido-5-chloro-2-fluorophenyl)-3-oxo-3,5,6,7,8,9- |
| | hexahydro-2H-6,9-epiminocyclohepta[c]pyridazine-10-carboxamide |
| 237 | (6S,9R)-N-(4-(Benzyloxy)-5-chloro-2-fluorophenyl)-3-oxo-3,5,6,7,8,9- |
| | hexahydro-2H-6,9-epiminocyclohepta[c]pyridazine-10-carboxamide |
| 238 | (6S,9R)-N-(5-(Benzo[c][1,2,5]oxadiazol-5-yl)-4-chloropyridin-2-yl)-3-oxo- |
| | 3,5,6,7,8,9-hexahydro-2H-6,9-epiminocyclohepta[c]pyridazine-10- |
| | carboxamide |
| 239 | (6S,9R)-N-(5-Chloro-4-(6-cyclobutoxypyridin-3-yl)-2-fluorophenyl)-3-oxo- |
| | 3,5,6,7,8,9-hexahydro-2H-6,9-epiminocyclohepta[c]pyridine-10-carboxamide |
| 240 | (5R,8S)-N-(5-Chloro-2-fluoro-4-(6-(2,2,2-trifluoroethoxy)pyridin-3-yl)phenyl)- |
| | 1-fluoro-6,7,8,9-tetrahydro-5H-5,8-epiminocyclohepta[c]pyridine-10- |
| | carboxamide |
| 241 | (6S,9R)-N-(5-Chloro-2-fluoro-4-(6-(2,2,2-trifluoroethoxy)pyridin-3-yl)phenyl)- |
| | 3-oxo-3,5,6,7,8,9-hexahydro-2H-6,9-epiminocyclohepta[c]pyridine-10- |
| | carboxamide |
| 242 | (5R,8S)-N-(2,5-Difluoro-4-(6-fluoropyridin-3-yl)phenyl)-1-fluoro-6,7,8,9- |
| | tetrahydro-5H-5,8-epiminocyclohepta[c]pyridine-10-carboxamide |
| 243 | (6S,9R)-N-(2,5-Difluoro-4-(6-fluoropyridin-3-yl)phenyl)-3-oxo-3,5,6,7,8,9- |
| | hexahydro-2H-6,9-epiminocyclohepta[c]pyridine-10-carboxamide |
| 244 | (6S,9R)-N-(2,5-Difluoro-4-(6-fluoropyridin-3-yl)phenyl)-3-oxo-3,5,6,7,8,9- |
| | hexahydro-2H-6,9-epiminocyclohepta[c]pyridazine-10-carboxamide |
| 245 | (5R,8S)-N-(2,5-Difluoro-4-(6-methoxypyridin-3-yl)phenyl)-1-fluoro-6,7,8,9- |
| | tetrahydro-5H-5,8-epiminocyclohepta[c]pyridine-10-carboxamide |
| 246 | (6S,9R)-N-(2,5-Difluoro-4-(6-methoxypyridin-3-yl)phenyl)-3-oxo-3,5,6,7,8,9- |
| | hexahydro-2H-6,9-epiminocyclohepta[c]pyridine-10-carboxamide |
| 247 | (6S,9R)-N-(2,5-Difluoro-4-(6-methoxypyridin-3-yl)phenyl)-3-oxo-3,5,6,7,8,9- |
| | hexahydro-2H-6,9-epiminocyclohepta[c]pyridazine-10-carboxamide |
| 248 | (5R,8S)-N-(5-Chloro-2-fluoro-4-(5-methoxypyridin-3-yl)phenyl)-1-fluoro- |
| | 6,7,8,9-tetrahydro-5H-5,8-epiminocyclohepta[c]pyridine-10-carboxamide |
| 249 | (6S,9R)-N-(5-Chloro-2-fluoro-4-(5-methoxypyridin-3-yl)phenyl)-3-oxo- |
| | 3,5,6,7,8,9-hexahydro-2H-6,9-epiminocyclohepta[c]pyridine-10-carboxamide |
| 250 | (5R,8S)-N-(2-Chloro-5-fluoro-[1,1′-biphenyl]-4-yl)-1-fluoro-6,7,8,9-tetrahydro- |
| | 5H-5,8-epiminocyclohepta[c]pyridine-10-carboxamide |
| 251 | (6S,9R)-N-(2-Chloro-5-fluoro-[1,1′-biphenyl]-4-yl)-3-oxo-3,5,6,7,8,9- |
| | hexahydro-2H-6,9-epiminocyclohepta[c]pyridine-10-carboxamide |
| 252 | (6S,9R)-N-(5-Chloro-4-(2,5-difluoropyridin-3-yl)-2-fluorophenyl)-3-oxo- |
| | 3,5,6,7,8,9-hexahydro-2H-6,9-epiminocyclohepta[c]pyridine-10-carboxamide |
| 253 | (5R,8S)-N-(5-(benzo[c][1,2,5]oxadiazol-5-yl)-2-fluoro-4- |
| | (trifluoromethyl)phenyl)-1-fluoro-6,7,8,9-tetrahydro-5H-5,8- |
| | epiminocyclohepta[c]pyridine-10-carboxamide |
| 254 | (6S,9R)-N-(5-(Benzo[c][1,2,5]oxadiazol-5-yl)-2-fluoro-4- |
| | (trifluoromethyl)phenyl)-3-oxo-3,5,6,7,8,9-hexahydro-2H-6,9- |
| | epiminocyclohepta[c]pyridazine-10-carboxamide |
| 255 | (6S,9R)-N-(2-Fluoro-5-(5-fluoropyridin-3-yl)-4-(trifluoromethyl)phenyl)-3-oxo- |
| | 3,5,6,7,8,9-hexahydro-2H-6,9-epiminocyclohepta[c]pyridine-10-carboxamide |
| 256 | (5R,8S)-1-fluoro-N-(2-fluoro-5-(5-fluoropyridin-3-yl)-4-(trifluoromethyl)phenyl)- |
| | 6,7,8,9-tetrahydro-5H-5,8-epiminocyclohepta[c]pyridine-10-carboxamide |
| 257 | (6S,9R)-N-(2-Fluoro-5-(5-fluoropyridin-3-yl)-4-(trifluoromethyl)phenyl)-3-oxo- |
| | 3,5,6,7,8,9-hexahydro-2H-6,9-epiminocyclohepta[c]pyridazine-10- |
| | carboxamide |
| 258 | (6S,9R)-N-(4-Fluoro-6-(trifluoromethyl)-[1,1′-biphenyl]-3-yl)-3-oxo-3,5,6,7,8,9- |
| | hexahydro-2H-6,9-epiminocyclohepta[c]pyridazine-10-carboxamide |
| 259 | (6S,9R)-N-(5-(Benzo[c][1,2,5]oxadiazol-5-yl)-2-fluoro-4- |
| | (trifluoromethyl)phenyl)-3-oxo-3,5,6,7,8,9-hexahydro-2H-6,9- |
| | epiminocyclohepta[c]pyridine-10-carboxamide |
| 260 | (5R,8S)-N-(5-Chloro-4-(2,5-difluoropyridin-3-yl)-2-fluorophenyl)-1-fluoro- |
| | 6,7,8,9-tetrahydro-5H-5,8-epiminocyclohepta[c]pyridine-10-carboxamide |
| 261 | (6S,9R)-N-(5-Chloro-4-(5,6-difluoropyridin-3-yl)-2-fluorophenyl)-3-oxo- |
| | 3,5,6,7,8,9-hexahydro-2H-6,9-epiminocyclohepta[c]pyridine-10-carboxamide |
| 262 | (5R,8S)-N-(5-chloro-4-(5,6-difluoropyridin-3-yl)-2-fluorophenyl)-1-fluoro- |
| | 6,7,8,9-tetrahydro-5H-5,8-epiminocyclohepta[c]pyridine-10-carboxamide |
| 263 | (5R,8S)-N-(5-Chloro-4-(6-ethoxypyridin-3-yl)-2-fluorophenyl)-1-fluoro-6,7,8,9- |
| | tetrahydro-5H-5,8-epiminocyclohepta[c]pyridine-10-carboxamide |
| 264 | (5R,8S)-N-(5-Chloro-2-fluoro-4-(2-methoxypyridin-4-yl)phenyl)-1-fluoro- |
| | 6,7,8,9-tetrahydro-5H-5,8-epiminocyclohepta[c]pyridine-10-carboxamide |
| 265 | (6S,9R)-N-(5-Chloro-2-fluoro-4-(2-methoxypyridin-4-yl)phenyl)-3-oxo- |
| | 3,5,6,7,8,9-hexahydro-2H-6,9-epiminocyclohepta[c]pyridine-10-carboxamide |
| 266 | (6S,9R)-N-(5-Chloro-2-fluoro-4-(2-fluoropyridin-4-yl)phenyl)-3-oxo- |
| | 3,5,6,7,8,9-hexahydro-2H-6,9-epiminocyclohepta[c]pyridazine-10- |
| | carboxamide |
| 267 | (6S,9R)-N-(5-Chloro-2-fluoro-4-(2-fluoropyridin-4-yl)phenyl)-3-oxo- |
| | 3,5,6,7,8,9-hexahydro-2H-6,9-epiminocyclohepta[c]pyridine-10-carboxamide |
| 268 | (5R,8S)-N-(5-Chloro-2-fluoro-4-(2-fluoropyridin-4-yl)phenyl)-1-fluoro-6,7,8,9- |
| | tetrahydro-5H-5,8-epiminocyclohepta[c]pyridine-10-carboxamide |
| 269 | (6S,9R)-N-(4-([1,2,5]Oxadiazolo[3,4-b]pyridin-6-yl)-2-fluoro-5- |
| | (trifluoromethyl)phenyl)-3-oxo-3,5,6,7,8,9-hexahydro-2H-6,9- |
| | epiminocyclohepta[c]pyridazine-10-carboxamide |
| 270 | (6S,9R)-N-(4-(Benzo[c]isothiazol-6-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 271 | (6S,9R)-N-(5-Chloro-2-fluoro-4-(pyrazin-2-yl)phenyl)-3-oxo-3,5,6,7,8,9- |
| | hexahydro-2H-6,9-epiminocyclohepta[c]pyridazine-10-carboxamide |
| 272 | (6S,9R)-N-(5-Chloro-4-(6-ethoxypyridin-3-yl)-2-fluorophenyl)-3-oxo- |
| | 3,5,6,7,8,9-hexahydro-2H-6,9-epiminocyclohepta[c]pyridine-10-carboxamide |
| 273 | (6S,9R)-N-(5-Chloro-2-fluoro-4-(6-isopropoxypyridin-3-yl)phenyl)-3-oxo- |
| | 3,5,6,7,8,9-hexahydro-2H-6,9-epiminocyclohepta[c]pyridine-10-carboxamide |
| 274 | (5R,8S)-N-(5-Chloro-2-fluoro-4-(6-isopropoxypyridin-3-yl)phenyl)-1-fluoro- |
| | 6,7,8,9-tetrahydro-5H-5,8-epiminocyclohepta[c]pyridine-10-carboxamide |
| 275 | (6S,9R)-N-(5-Chloro-2-fluoro-4-(6-(oxetan-3-yloxy)pyridin-3-yl)phenyl)-3-oxo- |
| | 3,5,6,7,8,9-hexahydro-2H-6,9-epiminocyclohepta[c]pyridazine-10- |
| | carboxamide |
| 276 | (5R,8S)-N-(5-Chloro-2-fluoro-4-(2-fluoro-6-methoxypyridin-3-yl)phenyl)-1- |
| | fluoro-6,7,8,9-tetrahydro-5H-5,8-epiminocyclohepta[c]pyridine-10- |
| | carboxamide |
| 277 | (6S,9R)-N-(4-([1,2,5]Thiadiazolo[3,4-b]pyridin-6-yl)-5-chloro-2-fluorophenyl)- |
| | 3-oxo-3,5,6,7,8,9-hexahydro-2H-6,9-epiminocyclohepta[c]pyridazine-10- |
| | carboxamide |
| 278 | (6S,9R)-N-(5-Chloro-2-fluoro-4-(6-(methoxymethyl)pyridin-3-yl)phenyl)-3-oxo- |
| | 3,5,6,7,8,9-hexahydro-2H-6,9-epiminocyclohepta[c]pyridazine-10- |
| | carboxamide |
| 279 | (6S,9R)-N-(5-Chloro-4-(5,6-difluoropyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 280 | (6S,9R)-N-(5-Chloro-2-fluoro-4-(5-(methoxymethyl)pyridin-3-yl)phenyl)-3-oxo- |
| | 3,5,6,7,8,9-hexahydro-2H-6,9-epiminocyclohepta[c]pyridazine-10- |
| | carboxamide |
| 281 | (6S,9R)-N-(5-Chloro-2-fluoro-4-(2-methoxypyrimidin-5-yl)phenyl)-3-oxo- |
| | 3,5,6,7,8,9-hexahydro-2H-6,9-epiminocyclohepta[c]pyridazine-10- |
| | carboxamide |
| 282 | (6S,9R)-N-(5-Chloro-2-fluoro-4-(1-methyl-1H-pyrrolo[2,3-b]pyridin-4- |
| | yl)phenyl)-3-oxo-3,5,6,7,8,9-hexahydro-2H-6,9- |
| | epiminocyclohepta[c]pyridazine-10-carboxamide |
| 283 | (5R,8S)-N-(4,5-Dichloro-2-fluorophenyl)-1-fluoro-4-(2-methoxyethoxy)- |
| | 6,7,8,9-tetrahydro-5H-5,8-epiminocyclohepta[c]pyridine-10-carboxamide |
| 284 | ±(5-endo)-N-(4,5-dichloro-2-fluorophenyl)-1,5-difluoro-6,7,8,9-tetrahydro-5H- |
| | 6,9-epiminocyclohepta[c]pyridine-10-carboxamide |
| 285 | (5R,8S)-N-(5-cyclohexyl-1,3,4-thiadiazol-2-yl)-1-fluoro-6,7,8,9-tetrahydro-5H- |
| | 5,8-epiminocyclohepta[c]pyridine-10-carboxamide |
| 286 | (6S,9R)-N-(2,5-Difluoro-4-(6-(trifluoromethyl)pyridin-3-yl)phenyl)-3-oxo- |
| | 3,5,6,7,8,9-hexahydro-2H-6,9-epiminocyclohepta[c]pyridazine-10- |
| | carboxamide |
| 287 | (±)-N-(4,5-dichloro-2-fluorophenyl)-N,1-dimethyl-2-oxo-2,5,6,7,8,9-hexahydro- |
| | 1H-5,8-epiminocyclohepta[b]pyridine-10-carboxamide |
| 288 | (±)-N-(4,5-dichloro-2-fluorophenyl)-N,1-dimethyl-2-oxo-2,5,6,7,8,9-hexahydro- |
| | 1H-5,8-epiminocyclohepta[b]pyridine-10-carboxamide |
| 289 | (±)-N-(4-(Benzo[c][1,2,5]oxadiazol-5-yl)-5-chloro-2-fluorophenyl)-1,9,9- |
| | trifluoro-6,7,8,9-tetrahydro-5H-5,8-epiminocyclohepta[c]pyridine-10- |
| | carboxamide |
| 290 | (±)-N-(5-Chloro-2-fluoro-4-(6-(2,2,2-trifluoroethoxy)pyridin-3-yl)phenyl)-1,9,9- |
| | trifluoro-6,7,8,9-tetrahydro-5H-5,8-epiminocyclohepta[c]pyridine-10- |
| | carboxamide |
| 291 | (6S,9R)-N-(5-Chloro-2-fluoro-4-(6-(2-fluoroethoxy)pyridin-3-yl)phenyl)-3-oxo- |
| | 3,5,6,7,8,9-hexahydro-2H-6,9-epiminocyclohepta[c]pyridazine-10- |
| | carboxamide |
| 292 | (6S,9R)-N-(5-Chloro-4-(6-(3,3-difluorocyclobutoxy)pyridin-3-yl)-2- |
| | fluorophenyl)-3-oxo-3,5,6,7,8,9-hexahydro-2H-6,9-epiminocyclohepta- |
| | [c]pyridazine-10-carboxamide |
| 293 | (6S,9R)-N-(5-Chloro-2-fluoro-4-(((6-fluoropyridin-3-yl)oxy)methyl)phenyl)-3- |
| | oxo-3,5,6,7,8,9-hexahydro-2H-6,9-epiminocyclohepta[c]pyridazine-10- |
| | carboxamide |
| 294 | (6S,9R)-N-(4-(benzo[c][1,2,5]oxadiazol-5-yloxy)-5-chloro-2-fluorophenyl)-3- |
| | oxo-3,5,6,7,8,9-hexahydro-2H-6,9-epiminocyclohepta[c]pyridazine-10- |
| | carboxamide |
| 295 | (5S,8R,9R)-N-(5-chloro-2-fluoro-4-(trifluoromethyl)phenyl)-1,9-difluoro- |
| | 6,7,8,9-tetrahydro-5H-5,8-epiminocyclohepta[c]pyridine-10-carboxamide |
| 296 | (5R,8S,9S)-N-(5-chloro-2-fluoro-4-(trifluoromethyl)phenyl)-1,9-difluoro- |
| | 6,7,8,9-tetrahydro-5H-5,8-epiminocyclohepta[c]pyridine-10-carboxamide |
| 297 | (5R,8S)-N-(5-Chloro-2-fluoro-4-(3-methoxypyridin-4-yl)phenyl)-1-fluoro- |
| | 6,7,8,9-tetrahydro-5H-5,8-epiminocyclohepta[c]pyridine-10-carboxamide |
| 298 | (6S,9R)-N-(5-Chloro-2-fluoro-4-(3-methoxypyridin-4-yl)phenyl)-3-oxo- |
| | 3,5,6,7,8,9-hexahydro-2H-6,9-epiminocyclohepta[c]pyridine-10-carboxamide |
| 299 | (6S,9R)-N-(5-chloro-2-fluoro-4-(3-methoxypyridin-4-yl)phenyl)-3-oxo- |
| | 3,5,6,7,8,9-hexahydro-2H-6,9-epiminocyclohepta[c]pyridazine-10- |
| | carboxamide |
| 300 | (5R,8S)-N-(5-chloro-2-fluoro-4-(6-(2-(methylamino)ethoxy)pyridin-3- |
| | yl)phenyl)-1-fluoro-6,7,8,9-tetrahydro-5H-5,8-epiminocyclohepta[c]pyridine- |
| | 10-carboxamide |
| 301 | (5R,8S)-N-(5-Chloro-4-(6-(2-(dimethylamino)ethoxy)pyridin-3-yl)-2- |
| | fluorophenyl)-1-fluoro-6,7,8,9-tetrahydro-5H-5,8- |
| | epiminocyclohepta[c]pyridine-10-carboxamide |
| 302 | (6S,9R)-N-(4-((Benzo[c][1,2,5]oxadiazol-5-yloxy)methyl)-5-chloro-2- |
| | fluorophenyl)-3-oxo-3,5,6,7,8,9-hexahydro-2H-6,9- |
| | epiminocyclohepta[c]pyridazine-10-carboxamide |
| 303 | (6S,9R)-N-(5-Chloro-2-fluoro-4-(6-(2-methoxyethoxy)pyridin-3-yl)phenyl)-3- |
| | oxo-3,5,6,7,8,9-hexahydro-2H-6,9-epiminocyclohepta[c]pyridazine-10- |
| | carboxamide |
| 304 | (6S,9R)-N-(5-Chloro-2-fluoro-4-(6-((1s,3S)-3-methoxycyclobutoxy)pyridin-3- |
| | yl)phenyl)-3-oxo-3,5,6,7,8,9-hexahydro-2H-6,9- |
| | epiminocyclohepta[c]pyridazine-10-carboxamide |
| 305 | (6S,9R)-N-(5-Chloro-4-(6-(2,2-difluoroethoxy)pyridin-3-yl)-2-fluorophenyl)-3- |
| | oxo-3,5,6,7,8,9-hexahydro-2H-6,9-epiminocyclohepta[c]pyridazine-10- |
| | carboxamide |
| 306 | (6S,9R)-N-(5-chloro-2-fluoro-4-(6-((1r,3R)-3-methoxycyclobutoxy)pyridin-3- |
| | yl)phenyl)-3-oxo-3,5,6,7,8,9-hexahydro-2H-6,9- |
| | epiminocyclohepta[c]pyridazine-10-carboxamide |
| 307 | (6S,9R)-N-(5-Chloro-4-(6-((1,3-difluoropropan-2-yl)oxy)pyridin-3-yl)-2- |
| | fluorophenyl)-3-oxo-3,5,6,7,8,9-hexahydro-2H-6,9- |
| | epiminocyclohepta[c]pyridazine-10-carboxamide |
| 308 | (6S,9R)-N-(3,4-Dichloro-2,6-difluorophenyl)-3-oxo-3,5,6,7,8,9-hexahydro-2H- |
| | 6,9-epiminocyclohepta[c]pyridazine-10-carboxamide |
| 309 | (6S,9R)-N-(5-Chloro-2-fluoro-4-(2-(2,2,2-trifluoroethoxy)pyridin-4-yl)phenyl)- |
| | 3-oxo-3,5,6,7,8,9-hexahydro-2H-6,9-epiminocyclohepta[c]pyridine-10- |
| | carboxamide |
| 310 | (6S,9R)-N-(5-Chloro-2-fluoro-4-(2-(2,2,2-trifluoroethoxy)pyridin-4-yl)phenyl)- |
| | 3-oxo-3,5,6,7,8,9-hexahydro-2H-6,9-epiminocyclohepta[c]pyridazine-10- |
| | carboxamide |
| 311 | (6S,9R)-N-(5-Chloro-2-fluoro-4-(5-(fluoromethyl)pyridin-3-yl)phenyl)-3-oxo- |
| | 3,5,6,7,8,9-hexahydro-2H-6,9-epiminocyclohepta[c]pyridazine-10- |
| | carboxamide |
| 312 | (6S,9R)-N-(5-Chloro-2-fluoro-4-(6-(trifluoromethyl)pyridin-3-yl)phenyl)-3-oxo- |
| | 3,5,6,7,8,9-hexahydro-2H-6,9-epiminocyclohepta[c]pyridine-10-carboxamide |
| 313 | (6S,9R)-N-(5-Chloro-2-fluoro-4-(6-(trifluoromethyl)pyridin-3-yl)phenyl)-3-oxo- |
| | 3,5,6,7,8,9-hexahydro-2H-6,9-epiminocyclohepta[c]pyridazine-10- |
| | carboxamide |
| 314 | (6S,9R)-N-(4-([1,2,5]oxadiazolo[3,4-b]pyridin-5-yl)-5-chloro-2-fluorophenyl)-3- |
| | oxo-3,5,6,7,8,9-hexahydro-2H-6,9-epiminocyclohepta[c]pyridazine-10- |
| | carboxamide |
| 315 | (6S,9R)-N-(5-Chloro-4-(6-(difluoromethoxy)pyridin-3-yl)-2-fluorophenyl)-3- |
| | oxo-3,5,6,7,8,9-hexahydro-2H-6,9-epiminocyclohepta[c]pyridazine-10- |
| | carboxamide |
| 316 | (6S,9R)-N-(5-Chloro-2-fluoro-4-(((5-fluoropyridin-3-yl)oxy)methyl)phenyl)-3- |
| | oxo-3,5,6,7,8,9-hexahydro-2H-6,9-epiminocyclohepta[c]pyridazine-10- |
| | carboxamide |
| 317 | (6S,9R)-N-(5-Chloro-2-fluoro-4-(6-((1r,3R)-3-methoxycyclobutoxy)pyridin-3- |
| | yl)phenyl)-3-oxo-3,5,6,7,8,9-hexahydro-2H-6,9-epiminocyclohepta[c]pyridine- |
| | 10-carboxamide |
| 318 | (6S,9R)-N-(5-Chloro-4-(6-(cyclopropylmethoxy)pyridin-3-yl)-2-fluorophenyl)-3- |
| | oxo-3,5,6,7,8,9-hexahydro-2H-6,9-epiminocyclohepta[c]pyridazine-10- |
| | carboxamide |
| 319 | (6S,9R)-N-(5-Chloro-2-fluoro-4-(6-isobutoxypyridin-3-yl)phenyl)-3-oxo- |
| | 3,5,6,7,8,9-hexahydro-2H-6,9-epiminocyclohepta[c]pyridazine-10- |
| | carboxamide |
| 320 | (6S,9R)-N-(5-Chloro-2-fluoro-4-(6-(3,3,3-trifluoropropoxy)pyridin-3-yl)phenyl)- |
| | 3-oxo-3,5,6,7,8,9-hexahydro-2H-6,9-epiminocyclohepta[c]pyridazine-10- |
| | carboxamide |
| 321 | (6S,9R)-N-(5-Chloro-2-fluoro-4-(6-((1s,3S)-3-methoxycyclobutoxy)pyridin-3- |
| | yl)phenyl)-3-oxo-3,5,6,7,8,9-hexahydro-2H-6,9-epiminocyclohepta[c]pyridine- |
| | 10-carboxamide |
| 322 | (6S,9R)-N-(5-Chloro-4-(6-(2,2-difluoroethoxy)pyridin-3-yl)-2-fluorophenyl)-3- |
| | oxo-3,5,6,7,8,9-hexahydro-2H-6,9-epiminocyclohepta[c]pyridine-10- |
| | carboxamide |
| 323 | (6S,9R)-N-(5-Chloro-2-fluoro-4-(7-fluorobenzo[c][1,2,5]oxadiazol-5-yl)phenyl)- |
| | 3-oxo-3,5,6,7,8,9-hexahydro-2H-6,9-epiminocyclohepta[c]pyridazine-10- |
| | carboxamide |
| 324 | (5R,8S)-N-(5-Chloro-4-(6-(difluoromethoxy)pyridin-3-yl)-2-fluorophenyl)-1- |
| | fluoro-6,7,8,9-tetrahydro-5H-5,8-epiminocyclohepta[c]pyridine-10- |
| | carboxamide |
| 325 | (6S,9R)-N-(5-Chloro-4-(6-(difluoromethoxy)pyridin-3-yl)-2-fluorophenyl)-3- |
| | oxo-3,5,6,7,8,9-hexahydro-2H-6,9-epiminocyclohepta[c]pyridine-10- |
| | carboxamide |
| 326 | (6S,9R)-N-(5-Chloro-2-fluoro-4-(6-fluorobenzo[c][1,2,5]oxadiazol-5-yl)phenyl)- |
| | 3-oxo-3,5,6,7,8,9-hexahydro-2H-6,9-epiminocyclohepta[c]pyridazine-10- |
| | carboxamide |
| 327 | (6S,9R)-N-(5-Chloro-2-fluoro-4-(4-fluorobenzo[c][1,2,5]oxadiazol-5-yl)phenyl)- |
| | 3-oxo-3,5,6,7,8,9-hexahydro-2H-6,9-epiminocyclohepta[c]pyridazine-10- |
| | carboxamide |
| 328 | (6S,9R)-N-(5-Chloro-2-fluoro-4-(5-fluoro-6-(2,2,2-trifluoroethoxy)pyridin-3- |
| | yl)phenyl)-3-oxo-3,5,6,7,8,9-hexahydro-2H-6,9- |
| | epiminocyclohepta[c]pyridazine-10-carboxamide |
| 329 | (5S,8R)-N-(4-(benzo[c][1,2,5]oxadiazol-5-yl)-5-chloro-2-fluorophenyl)-1,9,9- |
| | trifluoro-6,7,8,9-tetrahydro-5H-5,8-epiminocyclohepta[c]pyridine-10- |
| | carboxamide |
| 330 | (5R,8S)-N-(4-(benzo[c][1,2,5]oxadiazol-5-yl)-5-chloro-2-fluorophenyl)-1,9,9- |
| | trifluoro-6,7,8,9-tetrahydro-5H-5,8-epiminocyclohepta[c]pyridine-10- |
| | carboxamide |
| 331 | (6S,9R)-N-(5-Chloro-2-fluoro-4-(6-((1,1,3,3-tetrafluoropropan-2-yl)oxy)pyridin- |
| | 3-yl)phenyl)-3-oxo-3,5,6,7,8,9-hexahydro-2H-6,9- |
| | epiminocyclohepta[c]pyridazine-10-carboxamide |
| 332 | (6S,9R)-N-(5-Chloro-2-fluoro-4-(6-(3,3,3-trifluoropropoxy)pyridin-3-yl)phenyl)- |
| | 3-oxo-3,5,6,7,8,9-hexahydro-2H-6,9-epiminocyclohepta[c]pyridine-10- |
| | carboxamide |
| 333 | (6S,9R)-N-(5-Chloro-4-(5-(difluoromethyl)pyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 334 | (6S,9R)-N-(5-Chloro-4-(6-(cyclopropylmethoxy)pyridin-3-yl)-2-fluorophenyl)-3- |
| | oxo-3,5,6,7,8,9-hexahydro-2H-6,9-epiminocyclohepta[c]pyridine-10- |
| | carboxamide |
| 335 | (6S,9R)-N-(5-Chloro-2-fluoro-4-(6-isobutoxypyridin-3-yl)phenyl)-3-oxo- |
| | 3,5,6,7,8,9-hexahydro-2H-6,9-epiminocyclohepta[c]pyridine-10-carboxamide |
| 336 | (6S,9R)-N-(5-Chloro-4-(6-(cyclopentyloxy)pyridin-3-yl)-2-fluorophenyl)-3-oxo- |
| | 3,5,6,7,8,9-hexahydro-2H-6,9-epiminocyclohepta[c]pyridazine-10- |
| | carboxamide |
| 337 | (6S,9R)-N-(5-chloro-2-fluoro-4-(6-(((1r,4r)-4-methoxycyclohexyl)oxy)pyridin-3- |
| | yl)phenyl)-3-oxo-3,5,6,7,8,9-hexahydro-2H-6,9- |
| | epiminocyclohepta[c]pyridazine-10-carboxamide |
| 338 | (6S,9R)-N-(5-Chloro-4-(6-((2,2-difluorocyclopropyl)methoxy)pyridin-3-yl)-2- |
| | fluorophenyl)-3-oxo-3,5,6,7,8,9-hexahydro-2H-6,9- |
| | epiminocyclohepta[c]pyridazine-10-carboxamide |
| 339 | (5R,8S)-N-(5-(Benzo[c][1,2,5]oxadiazol-5-yl)-4-chloropyridin-2-yl)-1-fluoro- |
| | 6,7,8,9-tetrahydro-5H-5,8-epiminocyclohepta[c]pyridine-10-carboxamide |
| 340 | (6S,9R)-N-(5-Chloro-2-fluoro-4-(6-morpholinopyridin-3-yl)phenyl)-3-oxo- |
| | 3,5,6,7,8,9-hexahydro-2H-6,9-epiminocyclohepta[c]pyridazine-10- |
| | carboxamide |
| 341 | (6S,9R)-N-(4-(6-(Azetidin-1-yl)pyridin-3-yl)-5-chloro-2-fluorophenyl)-3-oxo- |
| | 3,5,6,7,8,9-hexahydro-2H-6,9-epiminocyclohepta[c]pyridazine-10- |
| | carboxamide |
| 342 | (6S,9R)-N-(5-Chloro-2-fluoro-4-(6-((tetrahydrofuran-3-yl)oxy)pyridin-3- |
| | yl)phenyl)-3-oxo-3,5,6,7,8,9-hexahydro-2H-6,9- |
| | epiminocyclohepta[c]pyridazine-10-carboxamide |
| 343 | (6S,9R)-N-(5-Chloro-2-fluoro-4-(6-(((1s,4s)-4-methoxycyclohexyl)oxy)pyridin- |
| | 3-yl)phenyl)-3-oxo-3,5,6,7,8,9-hexahydro-2H-6,9- |
| | epiminocyclohepta[c]pyridazine-10-carboxamide |

and enantiomers thereof, and mixtures of enantiomers thereof, including racemic mixtures, and pharmaceutically acceptable salts and solvates thereof.

In one preferred embodiment, the compound is selected from the following: 1-28, 31, 32, 35, 37-39, 41-44, 46-49, 51-53, 55-60, 63-89, 91-93, 96-118, 120-141, 143-150, 152-157, 159-173, 175-286, and 289-343.

In a more preferred embodiment, the compound is selected from the following: 1-11, 15-20, 20, 22-24, 26, 28, 31, 32, 35, 37, 39, 41, 42, 44, 46, 47, 49, 51, 52, 55, 59, 60, 63-67, 69, 70, 74-77, 79, 81, 82, 84-87, 92, 96-98, 100-105, 108-115, 120, 121, 123-127, 131, 132, 134, 136, 138, 139, 141, 144, 145, 147, 152-155, 157, 159, 162-173, 175-178, 182-185, 188-191, 193-195, 197-202, 205-223, 225-227, 229, 230, 232-235, 237-270, 272-279, 281, 282, 285, 286, 289-297, 301-307, and 309-343.

### Process

A further aspect of the invention relates to a process for preparing a compound of formula (Ie) as defined above, where Y is CH₂, and Rₐ, R_{b} and R₆ are H, said process comprising the steps of:
(i) treating a compound of formula I-1a with glyoxylic acid monohydrate to give a compound of formula I-1b;
(ii) treating said compound of formula I-1b with N₂H₄·H₂O to form a compound of formula I-1c;
(ii) removing the Boc protecting group from said compound of formula I-1c and reacting the compound so formed with triphosgene and a compound of formula I-1g in NEt₃/DCM to form a compound of formula (Ie):

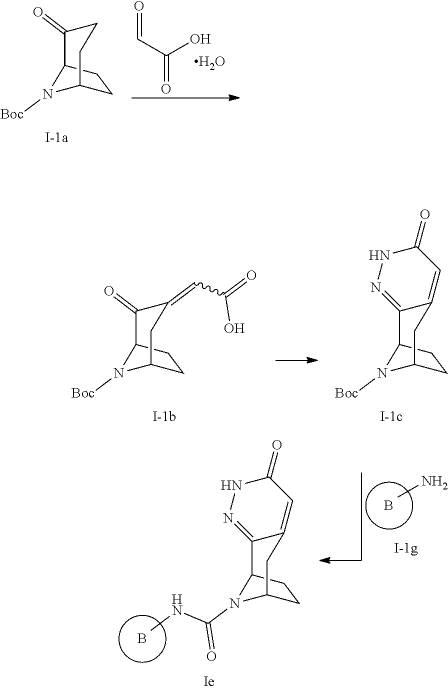

Other suitable protecting groups can also be used in place of the Boc group and will be familiar to the skilled person in the art.

Alternatively, the compound of formula I-1c is prepared by a process comprising the steps of:
(i) treating a compound of formula I-1b with morpholine to form a compound of formula I-1d;
(ii) treating said compound of formula I-1d with N₂H₄·H₂O to form a mixture comprising a compound of formula I-1c and a compound of formula I-1f; and
(iii) treating said mixture of compound I-1c and compound I-1f with NaOH in ethanol to yield a compound of formula I-1c:

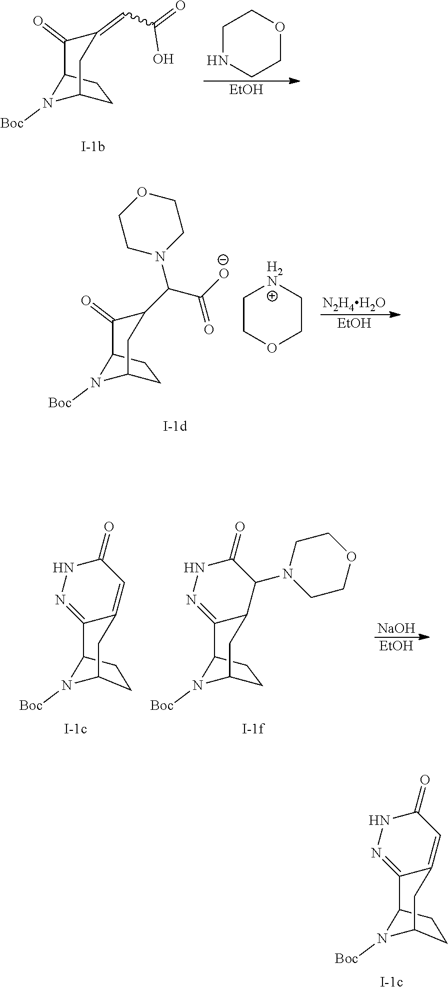

A further aspect of the invention relates to a process for preparing a compound of formula (Ia) as defined above where R₃ is an optionally substituted heteroaryl (“Het”), said process comprising the step of coupling a heteroaryl boronic acid with a bromophenyl intermediate as shown below:

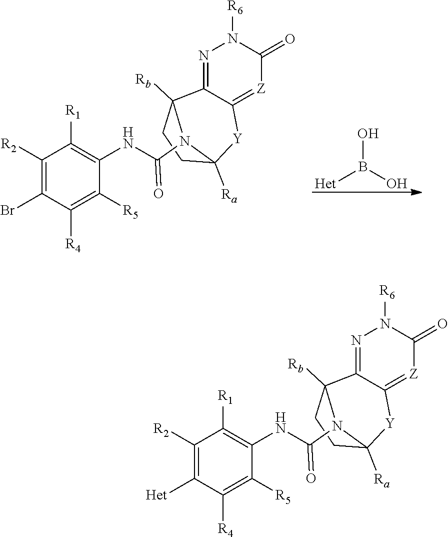

Preferably the coupling reaction takes place in the presence of a palladium complex, for example, Pd-170/XPhos (where XPhos=dicyclohexyl[2′,4′,6′-tris(propan-2-yl)[1,1′-biphenyl]-2-yl]phosphane). The skilled person would appreciate that other suitable catalysts would also be available.

Compounds which bear a pyrimidin-2(1H)-one group fused to the bicyclic nitrogen-containing core (e.g. 15-17 and 22-29 and the like), can be prepared by a process comprising the following steps:
(i) reacting a compound of formula I-3a with 1,1-dimethoxy-N,N-dimethylmethanamine to form a compound of formula I-3b;
(ii) treating said compound of formula I-3b with sodium ethoxide/urea to form a compound of formula I-3c;
(iii) removing the Boc protecting group from said compound of formula I-3c and reacting the compound so formed with triphosgene and a compound of formula I-3d in NEt₃/DCM:

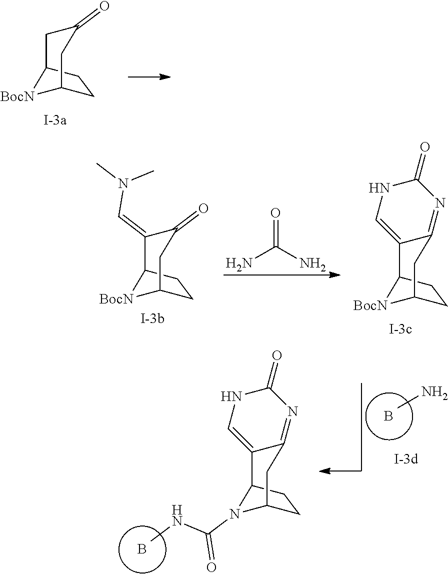

Further details of the above syntheses are set out in the accompanying examples.

### Therapeutic Applications

A further aspect of the invention relates to compounds as described herein for use in medicine. The compounds have particular use in the field of oncology, immuno-oncology, and immunology as described in more detail below. In a preferred embodiment, the compound of the invention modulates GPR65, and more preferably inhibits GPR65 signalling.

Yet another aspect of the invention relates to compounds as described herein for use as a medicament, preferably for use in treating or preventing a disorder selected from a proliferative disorder and an immune disorder.

Another aspect of the invention relates to compounds as described herein for use in treating or preventing asthma and/or chronic obstructive pulmonary disease (COPD). GPR65 variant/SNP (rs6574978) has been shown to be associated with asthma/COPD syndrome with almost GWAS significant p value (1.18×10 e−7) (Hardin, 2014). Furthermore, GPR65 activation by pH (pH is low/acidic in asthmatic lungs) promotes eosinophil viability in a cAMP-dependent manner, contributing to disease progression/exacerbation. It is further known that GPR65 KO mice have attenuated asthma symptoms (Kottyan, 2009).

Another aspect of the invention relates to compounds as described herein for use in treating or preventing acute respiratory distress syndrome (ARDS). GPR65 has been shown to be protective in a model of LPS-induced acute lung injury model (Tsurumaki, 2015).

One aspect of the invention relates to a compound as described herein for use in treating a proliferative disorder. Preferably, the proliferative disorder is a cancer or leukemia.

In one preferred embodiment, the cancer is a solid tumour and/or metastases thereof.

In another preferred embodiment, the cancer is selected from melanoma, renal cell carcinoma (RCC), gastric cancer, acute myeloid leukaemia (AML), triple negative breast cancer (TNBC), colorectal cancer, head and neck cancer, colorectal adenocarcinoma, pancreatic adenocarcinoma, lung cancer, sarcoma, ovarian cancer, and gliomas, preferably glioblastoma (GBM).

Without wishing to be bound by theory, it is understood that GPR65 modulators are capable of preventing the increase in cytoplasmic cAMP in tumour-associated macrophages (TAMs), natural killer (NK) cells and subsets of T cells that would typically result from their exposure to the acidic tumour microenvironment and concomitant GPR65 activation. This reduction in the level of cytoplasmic cAMP in turn reduces the levels of ICER and pro-inflammatory mediators such as CXCL10 and TNFα, preventing the polarization of TAMs and alteration of other immune cells that are associated with a non-inflammatory and tumour-permissive environment. Therefore, GPR65 modulators are expected to result in an increase in the visibility of the tumour to the immune system leading to increased immune-mediated tumour clearance. This suggests that modulation of GPR65 activity could be an effective treatment for cancer as stand-alone therapy or in combination with cancer immunotherapies (vaccines, agents that promote T cell mediated immune responses) or in patients that do not respond to immunomodulatory approaches such as PD1/PDL-1 blockade.

Another aspect of the invention relates to a compound as described herein for use in treating or preventing an immune disorder, preferably an autoimmune disease.

In one embodiment, the autoimmune disease is selected from psoriasis, psoriatic arthritis, rheumatoid arthritis (RA), multiple sclerosis (MS), systemic lupus erythematosus (SLE), autoimmune thyroiditis (Hashimoto's thyroiditis), Graves' disease, uveitis (including intermediate uveitis), ulcerative colitis, Crohn's disease, autoimmune uveoretinitis, systemic vasculitis, polymyositis-dermatomyositis, systemic sclerosis (scleroderma), Sjogren's Syndrome, ankylosing spondylitis and related spondyloarthropathies, sarcoidosis, autoimmune hemolytic anemia, immunological platelet disorders, autoimmune polyendocrinopathies, autoimmune myocarditis, type I diabetes and atopic dermatitis.

In a particularly preferred embodiment, the autoimmune disease is selected from psoriasis, psoriatic arthritis, ankylosing spondylitis, Crohn's disease, and multiple sclerosis (MS).

Without wishing to be bound by theory, it is understood that GPR65 modulators will prevent the upregulation of ICER in CD4+ T cells. This, in turn, is expected to prevent the ICER-associated suppression of IL-2 that biases CD4+ T cells toward the inflammatory Th17 phenotype associated with increased pathogenicity in the context of autoimmune disease. This is supported by the fact that mutations in the GPR65 locus are associated with several autoimmune diseases, such as multiple sclerosis, ankylosing spondylitis, inflammatory bowel disease, and Crohn's disease (Gaublomme, 2015). This suggests that modulation of GPR65 activity could be an effective treatment for autoimmune diseases.

Another aspect relates to a compound as described herein for use in treating or preventing a disorder caused by, associated with or accompanied by abnormal activity against GPR65.

Another aspect relates to a compound as described herein for use in treating or preventing a GPR65-associated disease or disorder.

Another aspect of the invention relates to a method of treating a disorder as described above comprising administering a compound as described herein to a subject.

Another aspect of the invention relates to a method of treating a GPR65-associated disease or disorder in a subject. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a subject having a disease state alleviated by modulation of GPR65 wherein the method comprises administering to the subject a therapeutically effective amount of a compound according to the invention.

Another aspect relates to a method of treating a disease state alleviated by modulation of GPR65, wherein the method comprises administering to a subject a therapeutically effective amount of a compound according to the invention.

Preferably, the subject is a mammal, more preferably a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately to determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition (see, e.g., Fingl et al, 1975, The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/day, commonly from about 100-1000 mg/day, preferably from about 150-700 mg/day and most preferably from about 250-500 mg/day or from 50-100 mg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “GPR65-related disease or disorder” refers to a disease or disorder characterized by inappropriate GPR65 activity. Inappropriate GPR65 activity refers to either an increase or decrease in GPR65 activity as measured by enzyme or cellular assays, for example, compared to the activity in a healthy subject. Inappropriate activity could also be due to overexpression of GPR65 in diseased tissue compared with healthy adjacent tissue.

Preferred diseases or disorders that the compounds described herein may be useful in preventing include proliferative disorders and immune disorders as described hereinbefore, as well as asthma and chronic obstructive pulmonary disease.

Thus, the present invention further provides use of compounds as defined herein in the preparation of a medicament for the treatment of a disease where it is desirable to modulate GPR65. Such diseases include proliferative disorders and immune disorders as described hereinbefore, as well as asthma and chronic obstructive pulmonary disease.

As used herein the phrase “preparation of a medicament” includes the use of the components of the invention directly as the medicament in addition to their use in any stage of the preparation of such a medicament.

In one preferred embodiment, the compound prevents the increase in cytoplasmic cAMP levels expected following GPR65 activation at acidic pH. This prevention of cAMP accumulation in turn prevents downstream signalling through ICER, as described above. The “Human GPR65 cyclic adenosine monophosphate (cAMP) Homogeneous Time Resolved Fluorescence (HTRF) antagonist assay”, or simply “cAMP assay”, as described in the accompanying examples, can be used to measure the potency of GPR65 modulators, which is expressed as the concentration of compound required to reduce the increase in cAMP concentration upon GPR65 activation by 50% (i.e. an IC₅₀).

In one preferred embodiment, the compound exhibits an IC₅₀ value in the cAMP assay of less than about 25 μM. More preferably, the compound exhibits an IC₅₀ value in the cAMP assay of less than about 10 μM, more preferably, less than about 5 μM, even more preferably, less than about 1 μM, even more preferably, less than about 0.1 μM.

In another preferred embodiment, the compound exhibits an hGPR65 IC50 value of less than <5 μM, more preferably less than <500 nM in the aforementioned assay.

In one preferred embodiment, the compound, or compound for use, according to the invention exhibits an IC50 of >500 nM and <5 μM in a Human GPR65 cyclic adenosine monophosphate (cAMP) Homogeneous Time Resolved Fluorescence (HTRF) antagonist assay as described in the accompanying examples. In one preferred embodiment, the compound is selected from those denoted “high” or “medium” in Table 1.

In a more preferred embodiment, the compound, or compound for use, according to the invention exhibits an IC50 of <500 nM in a Human GPR65 cAMP HTRF antagonist assay as described in the accompanying examples. In one preferred embodiment, the compound is selected from those denoted “high” in Table 1.

### Pharmaceutical Compositions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers, excipients or diluents therefor and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller. The carrier, or, if more than one be present, each of the carriers, must be acceptable in the sense of being compatible with the other ingredients of the formulation and not deleterious to the recipient.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound as described herein into conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentolate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkane alcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those compounds, and mixtures thereof. The terms used in the claims encompass these forms, provided said forms retain the appropriate functional activity (though not necessarily to the same degree).

The present invention also includes all suitable isotopic variations of the compound or a pharmaceutically acceptable salt thereof. An isotopic variation of a compound of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Atropisomers

Some of the compounds of the invention may exist as atropisomers. Atropisomers are stereoisomers arising because of hindered rotation about a single bond, where energy differences due to steric strain or other contributors create a barrier to rotation that is high enough to allow for isolation of individual conformers. The invention encompasses all such atropisomers. The invention also covers rotamers of the compounds.

### Prodrugs

The invention further includes the compounds of the present invention in prodrug form, i.e. covalently bonded compounds which release the active parent drug in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms. Preferably, the solvate is a hydrate.

### Combinations

A further aspect of the invention relates to a combination comprising a compound as described herein and one or more additional active agents. In a particularly preferred embodiment, the one or more compounds of the invention are administered in combination with one or more additional active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

In the context of cancer, compounds of the invention can be used in combination with immunotherapies such as cancer vaccines and/or with other immune-modulators such as agents that block the PD1/PDL-1 interaction. Other examples of agents for use in combination with the presently claimed compounds include immune modulators that block CTLA-4 or LAG-3. Thus, in one preferred embodiment, the additional active agent is an immunotherapy agent, more preferably a cancer immunotherapy agent. An “immunotherapy agent” refers to a treatment that uses the subject's own immune system to fight diseases such as cancer. For other disorders the compounds of the invention can be used in combination agents that block or decrease inflammation such as antibodies that target pro-inflammatory cytokines. The compounds of the invention can also be used in combination with other chemotherapy agents and/or in conjunction with radiotherapy.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

The dosage amount will further be modified according to the mode of administration of the compound. For example, to achieve an “effective amount” for acute therapy, parenteral administration of a compound is typically preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg; preferably between 0.1 and 20 mg, in a manner to maintain the concentration of drug in the plasma at a concentration effective to modulate GPR65. The compounds may be administered one to four times daily at a level to achieve a total daily dose of about 0.4 to about 400 mg. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 500 mg or about 0.1 to about 50 mg in a manner consistent with the condition of the patient. Preferably the oral dose would be about 0.5 to about 50 mg or about 0.5 to about 20 mg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

The invention is further described with reference to the following non-limiting examples.

### Examples

Where the preparation of starting materials is not described, these are commercially available, known in the literature, or readily obtainable by those skilled in the art using standard procedures. Where it is indicated that compounds were prepared analogously to earlier examples or intermediates, it will be appreciated by the skilled person that the reaction time, number of equivalents of reagents, solvent, concentration and temperature can be modified for each specific reaction and that it may be necessary or desirable to employ different work-up or purification techniques.

### General Schemes

### Abbreviations

A list of some common abbreviations is shown below—where other abbreviations are used which are not listed, these will be understood by the person skilled in the art.

AlBN: azobisisobutyronitrile; AcOH: acetic acid; BINAP: (2,2′-bis(diphenylphosphino)-1,1′-binaphthyl); Boc: tert-butyloxycarbonyl; br.: broad; CAN: Ceric ammonium nitrate; CBS: Corey-Bakshi-Shibata catalyst; CCl₄:tetrachloromethane; CMBP: (Tributylphosphoranylidene)acetonitrile; d: doublet; DAST: diethylaminosulfur trifluoride; DCM: dichloromethane; DIAD: diisopropyl azodicarboxylate; DIBAL: diisobutylaluminium hydride; DIPEA: N,N-diisopropylethylamine; DMF: N,N-dimethylformamide; DMSO: dimethylsulfoxide; dppf: 1,1′-bis(diphenylphosphino)ferrocene; (ES⁺): electrospray ionization positive mode; Et₃N: triethylamine; EtOAc: ethyl acetate; EtOH: ethanol; h: hours; hept: heptet; HPLC: high performance liquid chromatography; HCl: hydrochloric acid; Hz: hertz; J: coupling constant; I: litre; LDA: Lithium diisopropylamide; M: molar; m: multiplet [M+H]⁺: protonated molecular ion; MeCN: acetonitrile; MeOH: methanol; MHz: megahertz; min: minutes; ml: millilitres; MS: mass spectrometry; MTBE: methyl tert-butyl ether; m/z: mass-to-charge ratio; NBS: N-bromosuccinimide; NMO: N-Methylmorpholine N-oxide; NMP: N-Methyl-2-pyrrolidone; NMR: nuclear magnetic resonance; p: pentet; PDA: photodiode array; Pd₂(dba)₃: tris(dibenzylideneacetone)dipalladium; Pd(dppf)Cl₂: [1,1′-Bis(diphenylphosphino)ferrocene]dichloropalladium(II); Pd-161: AmPhos Pd(crotyl)Cl; Pd-172: SPhos Pd(crotyl)Cl; Pd-178: P(Cy)3 Pd(crotyl)Cl; PIDA: (Diacetoxyiodo)benzene; prep TLC: preparative thin layer chromatography; q: quintet; RT: room temperature; Rt: retention time; s: singlet; SFC: supercritical fluid chromatography; t: triplet; TBAF: Tetra-n-butylammonium fluoride; THF: tetrahydrofuran; TLC: thin layer chromatography; UPLC: ultra performance liquid chromatography; UV: ultra-violet; Xantphos: (9,9-dimethyl-9H-xanthene-4,5-diyl)bis(diphenylphosphane); XPhos: dicyclohexyl[2′,4′,6′-tris(propan-2-yl)[1,1′-biphenyl]-2-yl]phosphane Other abbreviations are intended to convey their generally accepted meaning.

### General Experimental Conditions

All starting materials and solvents were obtained either from commercial sources or prepared according to literature methods. The appropriate isocyanate and aniline starting materials were either commercially available and were obtained from, for example, Sigma Aldrich, Fluorochem or Enamine store, or were synthesised as described herein. The appropriate tricyclic amine starting materials were synthesised as described herein. Reaction mixtures were magnetically stirred and reactions performed at room temperature (approximately 20° C.) unless otherwise indicated.

Silica gel chromatography was performed on an automated flash chromatography system, such as CombiFlash Companion, CombiFlash Rf system or Reveleris X2 flash system using RediSep® Rf or Reveleris® or the GraceResolv™ pre-packed silica (230-400 mesh, 40-63 μm) cartridges.

Analytical UPLC-MS experiments to determine retention times and associated mass ions were performed using a Waters ACQUITY UPLC® H-Class system, equipped with ACQUITY PDA Detector and ACQUITY QDa mass spectrometer or Waters SQD mass spectrometer, running the analytical method described below.

Analytical LC-MS experiments to determine retention times and associated mass ions were performed using an Agilent 1200 series HPLC system coupled to an Agilent 1956, 6100 or 6120 series single quadrupole mass spectrometer running one of the analytical methods described below or a Shimadzu-2020-P2 system consisting of a Shimadzu LC-20AD series LC system and a Shimadzu-2020, single quadrupole mass spectrometer running one of the analytical methods described below

Analytical SFC experiments to determine retention times were performed using a Waters SFC system UPC2 system with a column temperature of 40° C. and a back pressure (ABPR) of 1750 psi using one of the analytical methods described.

Preparative HPLC purifications were performed either using a Waters Xbridge Prep OBD C18, 10 μm, 40×150 mm column using a gradient of MeCN and 0.1% ammonia in water or a gradient of MeCN and 0.1% formic acid in water. Fractions were collected following UV detection across all wavelengths with PDA and in some cases an SQD2 or ACQUITY QDa mass spectrometer.

Preparative SFC purifications were performed using either a Waters SFC prep 15 system, a Waters SFC prep 100 system or a Sepiatec Prep SFC 50 with either a: Phenomenex Lux® Cellulose-4, Column 1×25 cm, 5 μm particle size column or a Chiralpak® IG (Daicel Ltd.) column (1×25 cm, 5 μm particle size) or a Chiralpak IC 10×250 mm 5 μm particle size column or a Chiralpak IA 10×250 mm 5 μm particle size column or a Phenomenex Lux® 5 μm i-Cellulose-5, LC Column 250×21 mm or a Phenomenex Lux® A1 5 μm, LC Column 250×10 mm or a Chiralpak IH 10×250 mm, 5 m particle size column or a Chiralpak AY-H 10×250 mm, 5 m particle size column, flow rate 15-65 ml/min eluting with a mixture of CO₂ and co-solvent (MeOH, EtOH or IPA). Fractions were collected following UV detection at 210-400 nm using a PDA.

NMR spectra were recorded using either a Bruker Avance III HD 500 MHz instrument, a Bruker Avance Neo 400 MHz, Bruker Avance III 400 MHz instrument or a QOne AS400 400 MHz spectrometer using either residual non-deuterated solvent, or tetra-methylsilane as a reference

In the absence of the absolute stereochemistry being explicitly indicated through wedged and dashed bonds, chemical structures disclosed throughout the examples (for example, in the configuration (1.3) described hereinabove) are to be interpreted as depicting the racemate. For the avoidance of doubt, the invention encompasses the compounds in either configuration, as well as mixtures thereof.

### Separation of Enantiomers by Chiral Chromatography

It will be appreciated that the enantiomers of the compounds described above can be isolated using techniques well known in the art, including, but not limited to, chiral chromatography. For example, a racemic mixture can be dissolved in a solvent, for example, methanol, followed by separation by chiral SFC on a Waters prep 15 with UV detection by DAD at 210-400 nm, 40° C., 120 bar. The column was Chiralpak IG 10×250 mm, 5 m, flow rate 15 ml/min at 45% MeOH (0.1% DEA), 55% CO₂ to afford both enantiomers as the separated pure compounds.

### Analytical Methods

### Method 1—Basic 3 Min Method

Column: Waters ACQUITY UPLC® BEH C18, 1.7 μm, 2.1×30 mm at 40° C.
Detection: UV at 210-400 nm unless otherwise indicated, MS by electrospray ionisation
Solvents: A: 0.1% Ammonia in water, B: MeCN
Gradient:

| Time | % A | % B | Flow rate (ml/min) |
|---|---|---|---|
| 0.00 | 98 | 2 | 0.77 |
| 2.50 | 0 | 100 | 0.77 |
| 3.00 | 0 | 100 | 0.77 |

### Method 2—Acidic 3 Min Method 1

Column: Waters CSH C18, 1.7 μm, 2.1×30 mm at 40° C.
Detection: UV at 210-400 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 98 | 2 | 0.77 |
| 2.5 | 0 | 100 | 0.77 |
| 3 | 0 | 100 | 0.77 |

### Method 3—Acidic 5 Min Method 1

Column: Agilent EclipsePlus RRHD C18, 1.8 μm, 3.0×50 mm at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 90 | 10 | 0.8 |
| 0.50 | 90 | 10 | 0.8 |
| 4.00 | 10 | 90 | 0.8 |
| 4.50 | 0 | 100 | 0.8 |
| 4.51 | 90 | 10 | 0.8 |
| 5.00 | 90 | 10 | 0.8 |

### Method 4—Acidic 5 Min Method 2

Column: Waters Sunfire, 3.5 μm, 4.6×50 mm column at 25° C.
Detection: UV at 214 and 254 nm unless otherwise indicated, MS by electrospray ionisation
Solvents: A: 0.05% formic acid in water, B: 0.05% formic acid in MeCN
Gradient:

| Time | % A | % B | Flow rate (ml/min) |
|---|---|---|---|
| 0.00 | 85 | 15 | 1.0 |
| 0.50 | 85 | 15 | 1.0 |
| 4.00 | 0 | 100 | 1.0 |
| 4.50 | 0 | 100 | 1.0 |
| 4.51 | 85 | 15 | 1.0 |
| 5.00 | 85 | 15 | 1.0 |

### Method 5—Acidic 3 Min Method 2

Column: Waters CORTECS UPLC, C18, 1.6 μm, 2.1×50 mm column at 25° C.
Detection: UV at 210-400 nm unless otherwise indicated, MS by electrospray ionisation
Solvents: A: 0.1% formic acid in water, B: MeCN
Gradient:

| Time | % A | % B | Flow rate (ml/min) |
|---|---|---|---|
| 0.00 | 80 | 20 | 0.6 |
| 1.80 | 20 | 80 | 0.6 |
| 2.65 | 20 | 80 | 0.6 |
| 2.80 | 80 | 20 | 0.6 |
| 3.00 | 80 | 20 | 0.6 |

### Experimental Scheme 1

### Compound 1 (±)—N-(5-Chloro-2-fluoro-4-(trifluoromethyl)phenyl)-3-oxo-3,5,6,7,8,9-hexahydro-2H-6,9-epiminocyclohepta[c]pyridazine-10-carboxamide

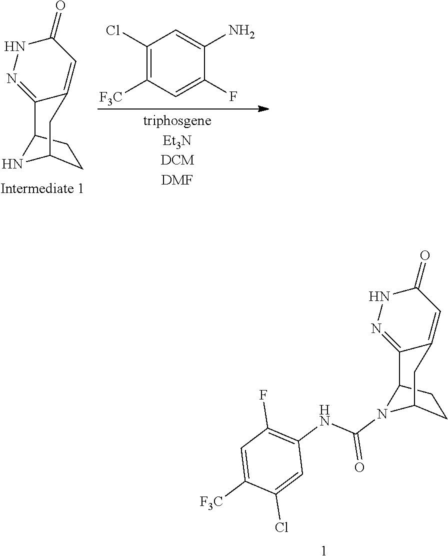 [SMILES: O=c1cc2c(n[nH]1)C1CCC(C2)N1 ; Nc1cc(Cl)c(C(F)(F)F)cc1F ; O=C(Nc1cc(Cl)c(C(F)(F)F)cc1F)N1C2CCC1c1n[nH]c(=O)cc1C2]

A solution of triphosgene (113 mg, 381 μmol) in DCM (5 ml) was prepared and cooled to 0° C. A separate solution of 5-chloro-2-fluoro-4-(trifluoromethyl)aniline (199 mg, 931 μmol) and Et₃N (708 μl, 5.08 mmol) in DCM (2 ml) was prepared and added dropwise to the stirred triphosgene solution. The reaction was allowed to stir at 0° C. for 1 h. (±)-2,5,6,7,8,9-hexahydro-3H-6,9-epiminocyclohepta[c]pyridazin-3-one (150 mg, 846 μmol) in DMF (6 ml) was added slowly, and the reaction was allowed to stir at RT for 3 h. The solvent was removed in vacuo. The product was purified by chromatography on silica gel (0-100% EtOAc/isohexane) to afford (±)—N-(5-chloro-2-fluoro-4-(trifluoromethyl)phenyl)-3-oxo-3,5,6,7,8,9-hexahydro-2H-6,9-epiminocyclohepta[c]pyridazine-10-carboxamide as a white powder. LCMS (Method 1) m/z 415.1, 417.1 (M+H)⁺ (ES⁺), at 1.14 min; ¹H NMR (400 MHz, DMSO-d6) δ 12.73 (s, 1H), 9.13 (s, 1H), 8.03 (d, J=6.8 Hz, 1H), 7.79 (d, J=11.0 Hz, 1H), 6.68 (s, 1H), 5.10 (d, J=5.7 Hz, 1H), 4.67 (d, J=6.1 Hz, 1H), 3.18 (dd, J=18.5, 5.3 Hz, 1H), 2.67 (s, 1H), 2.18 (dd, J=11.4, 6.3 Hz, 2H), 1.80 (t, J=9.4 Hz, 1H), 1.72-1.63 (m, 1H).

The following compounds were prepared using appropriate starting materials in an analogous procedure to that described in Experimental Scheme 1. Where the starting materials are not described in the literature, their synthesis is described below.

| | | Method | |
|---|---|---|---|
| Com- | | LCMS Rt | |
| pound | Structure | [M + H]⁺ | NMR |
| 2 | 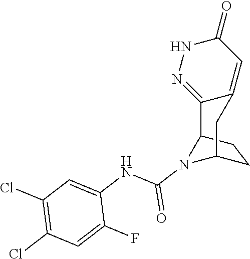 [SMILES: O=C(Nc1cc(Cl)c(Cl)cc1F)N1C2CCC1c1n[nH]c(=O)cc1C2] | Method 1 381.0, 383.4 at 1.01 min | ¹H NMR (400 MHz, DMSO-d6) δ 12.71 (s, 1H), 8.87 (s, 1H), 7.82 (d, J = 7.6 Hz, 1H), 7.68 (d, J = 10.3 Hz, 1H), 6.68 (s, 1H), 5.05 (d, J = 5.9 Hz, 1H), 4.62 (d, J = 6.2 Hz, 1H), 3.17 (p, J = 6.1 Hz, 1H), 2.64 (d, J = 18.2 Hz, 1H), 2.17 (dd, J = 12.0, 6.4 Hz, 2H), 1.79 (t, J = 9.7 Hz, 1H), 1.71-1.62 (m, 1H). |
| | (±)-N-(4,5-Dichloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 3 | 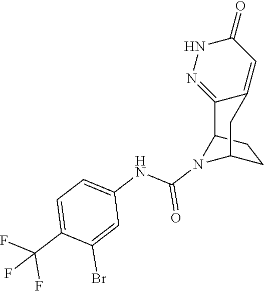 [SMILES: O=C(Nc1ccc(C(F)(F)F)c(Br)c1)N1C2CCC1c1n[nH]c(=O)cc1C2] | Method 1 443.3, 445.1 at 1.27 min | ¹H NMR (400 MHz, DMSO-d6) δ 12.74 (s, 1H), 9.27 (s, 1H), 8.08 (d, J = 1.9 Hz, 1H), 7.70 (d, J = 8.8 Hz, 1H), 7.67 (dd, J = 8.8, 1.9 Hz, 1H), 6.67 (s, 1H), 5.13 (d, J = 5.8 Hz, 1H), 4.68 (t, J = 6.1 Hz, 1H), 3.22-3.05 (m, 1H), 2.66 (d, J = 18.2 Hz, 1H), 2.27-2.08 (m, 2H), 1.90- 1.72 (m, 1H), 1.68 (t, J = 8.6 Hz, 1H) |
| | (±)-N-(3-Bromo-4- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 4 | 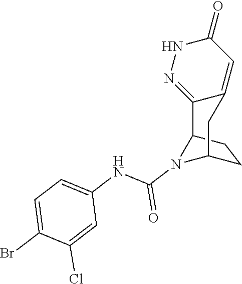 [SMILES: O=C(Nc1ccc(Br)c(Cl)c1)N1C2CCC1c1n[nH]c(=O)cc1C2] | Method 1 409.0, 411.5 at 1.17 min | ¹H NMR (400 MHz, DMSO-d6) δ 12.73 (s, 1H), 9.04 (s, 1H), 7.85 (d, J = 2.4 Hz, 1H), 7.60 (d, J = 8.9 Hz, 1H), 7.39 (dd, J = 8.9, 2.5 Hz, 1H), 6.67 (s, 1H), 5.10 (d, J = 5.8 Hz, 1H), 4.65 (t, J = 6.2 Hz, 1H), 3.20-3.08 (m, 1H), 2.65 (d, J = 18.2 Hz, 1H), 2.26-2.08 (m, 2H), 1.87- 1.74 (m, 1H), 1.71-1.59 (m, 1H). |
| | (±)-N-(4-Bromo-3- | | |
| | chlorophenyl)-3-oxo-3,5,6,7,8,9- | | |
| | hexahydro-2H-6,9- | | |
| | epiminocyclohepta[c]pyridazine- | | |
| | 10-carboxamide | | |
| 5 | 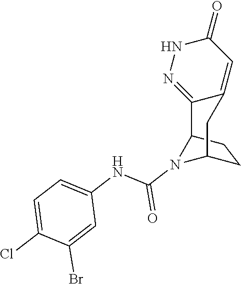 [SMILES: O=C(Nc1ccc(Cl)c(Br)c1)N1C2CCC1c1n[nH]c(=O)cc1C2] | Method 1 409.2, 411.1 at 1.16 min | ¹H NMR (400 MHz, DMSO-d6) δ 12.73 (s, 1H), 9.02 (s, 1H), 7.98 (d, J = 2.3 Hz, 1H), 7.51 (dd, J = 8.9, 2.3 Hz, 1H), 7.47 (d, J = 8.8 Hz, 1H), 6.67 (s, 1H), 5.10 (d, J = 5.9 Hz, 1H), 4.64 (t, J = 6.3 Hz, 1H), 3.20-3.07 (m, 1H), 2.65 (d, J = 18.2 Hz, 1H), 2.27-2.09 (m, 2H), 1.85- 1.73 (m, 1H), 1.73-1.58 (m, 1H). |
| | (±)-N-(3-Bromo-4- | | |
| | chlorophenyl)-3-oxo-3,5,6,7,8,9- | | |
| | hexahydro-2H-6,9- | | |
| | epiminocyclohepta[c]pyridazine- | | |
| | 10-carboxamide | | |
| 6 | 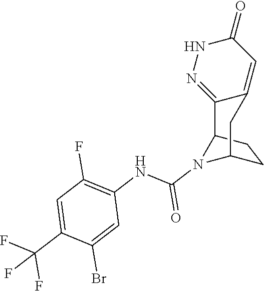 [SMILES: O=C(Nc1cc(Br)c(C(F)(F)F)cc1F)N1C2CCC1c1n[nH]c(=O)cc1C2] | Method 1 459.5, 461.4 at 1.25 min | ¹H NMR (400 MHz, DMSO-d6) δ 12.73 (s, 1H), 9.11 (s, 1H), 8.22-8.16 (m, 1H), 7.77 (d, J = 11.2 Hz, 1H), 6.68 (s, 1H), 5.09 (d, J = 5.6 Hz, 1H), 4.65 (d, J = 5.8 Hz, 1H), 3.18 (dd, J = 18.3, 5.3 Hz, 1H), 2.66 (d, J = 15.6 Hz, 1H), 2.18 (dd, J = 11.5, 6.4 Hz, 2H), 1.85-1.75 (m, 1H), 1.72-1.63 (m, 1H). |
| | (±)-N-(5-Bromo-2-fluoro-4- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 7 | 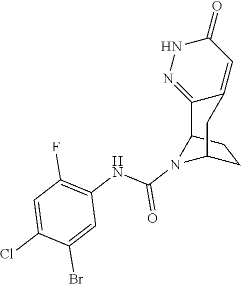 [SMILES: O=C(Nc1cc(Br)c(Cl)cc1F)N1C2CCC1c1n[nH]c(=O)cc1C2] | Method 1 427.0, 426.1 at 1.12 min © | ¹H NMR (400 MHz, DMSO-d6) δ 12.71 (s, 1H), 8.85 (s, 1H), 7.96-7.93 (m, 1H), 7.67 (d, J = 10.4 Hz, 1H), 6.68 (s, 1H), 5.05 (d, J = 5.9 Hz, 1H), 4.61 (d, J = 5.8 Hz, 1H), 3.21-3.11 (m, 1H), 2.64 (d, J = 18.4 Hz, 1H), 2.17 (dd, J = 12.1, 6.5 Hz, 2H), 1.84- 1.74 (m, 1H), 1.72-1.61 (m, 1H). |
| | (±)-N-(5-Bromo-4-chloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 8 | 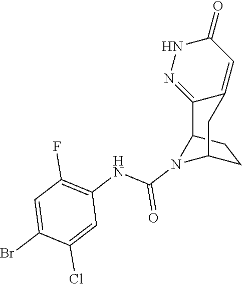 [SMILES: O=C(Nc1cc(Cl)c(Br)cc1F)N1C2CCC1c1n[nH]c(=O)cc1C2] | Method 1 426.8, 427.7 at 1.13 min | ¹H NMR (400 MHz, DMSO-d6) δ 12.71 (s, 1H), 8.86 (s, 1H), 7.86-7.82 (m, 1H), 7.76 (d, J = 10.1 Hz, 1H), 6.68 (s, 1H), 5.05 (d, J = 5.9 Hz, 1H), 4.62 (br s, 1H), 3.22-3.07 (m, 1H), 2.64 (d, J = 18.0 Hz, 1H), 2.26-2.10 (m, 2H), 1.87-1.74 (m, 1H), 1.68-1.65 (m, 1H). |
| | (±)-N-(4-Bromo-5-chloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 9 | 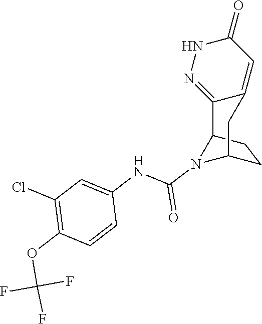 [SMILES: O=C(Nc1ccc(OC(F)(F)F)c(Cl)c1)N1C2CCC1c1n[nH]c(=O)cc1C2] | Method 1 415.2, 417.3 at 1.29 min | ¹H NMR (400 MHz, DMSO-d6) δ 12.73 (s, 1H), 9.12 (s, 1H), 7.87 (d, J = 2.6 Hz, 1H), 7.52 (dd, J = 9.1, 2.5 Hz, 1H), 7.47- 7.41 (m, 1H), 6.67 (s, 1H), 5.11 (d, J = 5.9 Hz, 1H), 4.65 (t, J = 6.3 Hz, 1H), 3.15 (dd, J = 18.2, 5.3 Hz, 1H), 2.65 (d, J = 18.1 Hz, 1H), 2.26-2.05 (m, 2H), 1.89-1.74 (m, 1H), 1.74-1.60 (m, 1H) |
| | (±)-N-(3-Chloro-4- | | |
| | (trifluoromethoxy)phenyl)-3-oxo- | | |
| | 3,5,6,7,8,9-hexahydro-2H-6,9- | | |
| | epiminocyclohepta[c]pyridazine- | | |
| | 10-carboxamide | | |
| 10 | 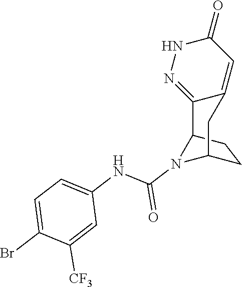 [SMILES: O=C(Nc1ccc(Br)c(C(F)(F)F)c1)N1C2CCC1c1n[nH]c(=O)cc1C2] | Method 1 443.2, 445.5 at 1.27 min | ¹H NMR (400 MHz, DMSO-d6) δ 12.73 (s, 1H), 9.18 (s, 1H), 8.06 (d, J = 1.8 Hz, 1H), 7.73 (d, J = 1.4 Hz, 2H), 6.67 (s, 1H), 5.12 (d, J = 5.9 Hz, 1H), 4.67 (t, J = 6.2 Hz, 1H), 3.15 (dd, J = 18.1, 5.4 Hz, 1H), 2.66 (d, J = 18.1 Hz, 1H), 2.29-2.05 (m, 2H), 1.88-1.74 (m, 1H), 1.67 (dd, J = 10.9, 6.3 Hz, 1H). |
| | (±)-N-(4-Bromo-3- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 11 | 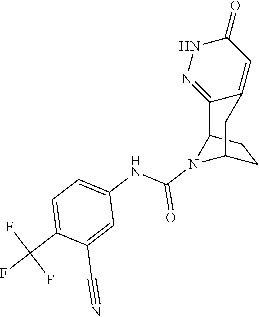 [SMILES: N#Cc1cc(NC(=O)N2C3CCC2c2n[nH]c(=O)cc2C3)ccc1C(F)(F)F] | Method 1 390.3 at 1.12 min | ¹H NMR (400 MHz, DMSO-d6) δ 12.82-12.64 (m, 1H), 9.48 (s, 1H), 8.23 (d, J = 2.2 Hz, 1H), 7.99-7.90 (m, 1H), 7.88 (d, J = 8.9 Hz, 1H), 6.68 (s, 1H), 5.15 (d, J = 5.8 Hz, 1H), 4.70 (t, J = 6.2 Hz, 1H), 3.16 (dd, J = 18.3, 5.3 Hz, 1H), 2.68 (d, J = 18.2 Hz, 1H), 2.24-2.09 (m, 2H), 1.87-1.73 (m, 1H), 1.75-1.60 (m, 1H). |
| | (±)-N-(3-Cyano-4- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 12 | 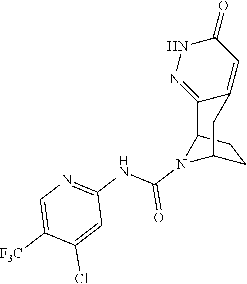 [SMILES: O=C(Nc1cc(Cl)c(C(F)(F)F)cn1)N1C2CCC1c1n[nH]c(=O)cc1C2] | Method 1 400.2, 402.2 at 1.27 min | ¹H NMR (400 MHz, DMSO-d6) δ 12.72 (s, 1H), 10.34 (s, 1H), 8.66 (d, J = 1.0 Hz, 1H), 8.15 (s, 1H), 6.67 (s, 1H), 5.21 (s, 1H), 4.75 (s, 1H), 3.17 (dd, J = 18.1, 5.5 Hz, 1H), 2.64 (d, J = 18.3 Hz, 1H), 2.16 (dq, J = 12.0, 7.1 Hz, 2H), 1.87-1.75 (m, 1H), 1.71-1.59 (m, 1H). |
| | (±)-N-(4-Chloro-5- | | |
| | (trifluoromethyl)pyridin-2-yl)-3- | | |
| | oxo-3,5,6,7,8,9-hexahydro-2H- | | |
| | 6,9-epiminocyclohepta- | | |
| | [c]pyridazine-10-carboxamide | | |
| 13^{a} | 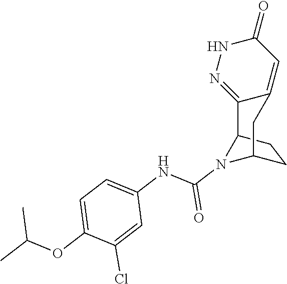 [SMILES: CC(C)Oc1ccc(NC(=O)N2C3CCC2c2n[nH]c(=O)cc2C3)cc1Cl] | Method 1 389.1, 391.3 at 1.12 min | ¹H NMR (400 MHz, DMSO-d6) δ 12.71 (s, 1H), 8.77 (s, 1H), 7.59 (d, J = 2.6 Hz, 1H), 7.32 (dd, J = 8.9, 2.6 Hz, 1H), 7.05 (d, J = 9.0 Hz, 1H), 6.66 (s, 1H), 5.07 (d, J = 5.9 Hz, 1H), 4.65- 4.58 (m, 1H), 4.51 (hept, J = 6.1 Hz, 1H), 3.21-3.06 (m, 1H), 2.63 (d, J = 18.2 Hz, 1H), 2.23- 2.05 (m, 2H), 1.82-1.71 (m, 1H), 1.72-1.59 (m, 1H), 1.25 (d, J = 6.0 Hz, 6H). |
| | (±)-N-(3-Chloro-4- | | |
| | isopropoxyphenyl)-3-oxo- | | |
| | 3,5,6,7,8,9-hexahydro-2H-6,9- | | |
| | epiminocyclohepta[c]pyridazine- | | |
| | 10-carboxamide | | |
| 14^{b} | 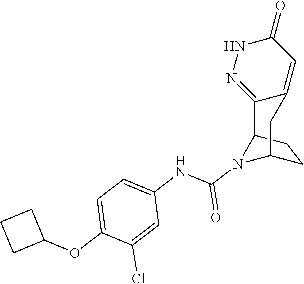 [SMILES: O=C(Nc1ccc(OC2CCC2)c(Cl)c1)N1C2CCC1c1n[nH]c(=O)cc1C2] | Method 1 401.3, 403.3 at 1.19 min | ¹H NMR (400 MHz, DMSO-d6) δ 12.71 (s, 1H), 8.74 (s, 1H), 7.59 (d, J = 2.5 Hz, 1H), 7.29 (dd, J = 8.9, 2.6 Hz, 1H), 6.87 (d, J = 9.0 Hz, 1H), 6.66 (s, 1H), 5.06 (d, J = 5.9 Hz, 1H), 4.71- 4.56 (m, 2H), 3.21-3.07 (m, 1H), 2.62 (d, J = 18.2 Hz, 1H), 2.46-2.34 (m, 2H), 2.25-1.95 (m, 4H), 1.85-1.71 (m, 2H), 1.71-1.55 (m, 2H). |
| | (±)-N-(3-Chloro-4- | | |
| | cyclobutoxyphenyl)-3-oxo- | | |
| | 3,5,6,7,8,9-hexahydro-2H-6,9- | | |
| | epiminocyclohepta[c]pyridazine- | | |
| | 10-carboxamide | | |
| 36 | 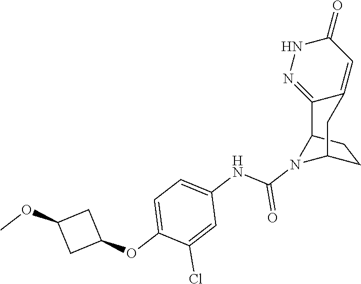 [SMILES: CO[C@H]1C[C@@H](Oc2ccc(NC(=O)N3C4CCC3c3n[nH]c(=O)cc3C4)cc2Cl)C1] | Method 1 431.3, 433.3 at 0.97 min | ¹H NMR (400 MHz, DMSO-d6) δ 12.71 (s, 1H), 8.76 (s, 1H), 7.59 (d, J = 2.6 Hz, 1H), 7.31 (dd, J = 9.0, 2.6 Hz, 1H), 6.89 (d, J = 9.0 Hz, 1H), 6.66 (s, 1H), 5.07 (d, J = 5.8 Hz, 1H), 4.61 (t, J = 6.1 Hz, 1H), 4.41-4.30 (m, 1H), 3.66-3.55 (m, 1H), 3.18- 3.08 (m, 4H), 2.89-2.77 (m, 2H), 2.62 (d, J = 18.2 Hz, 1H), 2.22-2.07 (m, 2H), 1.94-1.82 (m, 2H), 1.82-1.72 (m, 1H), 1.71-1.59 (m, 1H). |
| | (±)-N-(3-Chloro-4-((1s,3S)-3- | | |
| | methoxycyclobutoxy)phenyl)-3- | | |
| | oxo-3,5,6,7,8,9-hexahydro-2H- | | |
| | 6,9- | | |
| | epiminocyclohepta[c]pyridazine- | | |
| | 10-carboxamide | | |
| 42 | 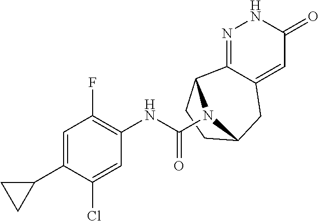 [SMILES: O=C(Nc1cc(Cl)c(C2CC2)cc1F)N1[C@H]2CC[C@@H]1c1n[nH]c(=O)cc1C2] | Method 1 389.3, 391.3 at 1.14 min | ¹H NMR (400 MHz, DMSO-d6) δ 12.70 (s, 1H), 8.67 (s, 1H), 7.54 (d, J = 7.4 Hz, 1H), 6.88 (d, J = 11.8 Hz, 1H), 6.67 (s, 1H), 5.03 (d, J = 5.9 Hz, 1H), 4.59 (t, J = 6.1 Hz, 1H), 3.17 (dd, J = 18.4, 5.2 Hz, 1H), 2.62 (d, J = 18.2 Hz, 1H), 2.25-2.01 (m, 3H), 1.84-1.73 (m, 1H), 1.73- 1.60 (m, 1H), 1.02-0.93 (m, 2H), 0.74-0.64 (m, 2H). |
| | (6S,9R)-N-(5-Chloro-4- | | |
| | cyclopropyl-2-fluorophenyl)-3- | | |
| | oxo-3,5,6,7,8,9-hexahydro-2H- | | |
| | 6,9-epiminocyclohepta- | | |
| | [c]pyridazine-10-carboxamide | | |
| 43^{(d)(e)} | 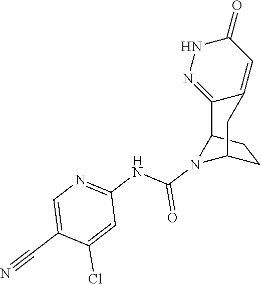 [SMILES: N#Cc1cnc(NC(=O)N2C3CCC2c2n[nH]c(=O)cc2C3)cc1Cl] | Method 1 355.2, 357.2 at 0.81 min^{(c)} | ¹H NMR (400 MHz, DMSO-d6) δ 12.73 (s, 1H), 10.43 (s, 1H), 8.79 (s, 1H), 8.13 (s, 1H), 6.67 (s, 1H), 5.21 (s, 1H), 4.75 (s, 1H), 3.17 (dd, J = 18.3, 5.4 Hz, 1H), 2.65 (d, J = 18.2 Hz, 1H), 2.26-2.09 (m, 2H), 1.87-1.75 (m, 1H), 1.73-1.57 (m, 1H). |
| | (±)-N-(4-Chloro-5-cyanopyridin- | | |
| | 2-yl)-3-oxo-3,5,6,7,8,9- | | |
| | hexahydro-2H-6,9- | | |
| | epiminocyclohepta[c]pyridazine- | | |
| | 10-carboxamide | | |
| 44^{(d)(e)} | 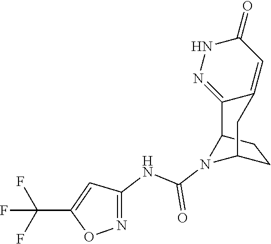 [SMILES: O=C(Nc1cc(C(F)(F)F)on1)N1C2CCC1c1n[nH]c(=O)cc1C2] | Method 1 353.8 at 0.78 min | ¹H NMR (400 MHz, DMSO-d6) δ 12.74 (s, 1H), 10.50 (s, 1H), 7.47 (d, J = 1.1 Hz, 1H), 6.68 (s, 1H), 5.16 (d, J = 5.6 Hz, 1H), 4.81-4.61 (m, 1H), 3.18-3.08 (m, 1H), 2.65 (d, J = 17.9 Hz, 1H), 2.26-2.08 (m, 2H), 1.86- 1.74 (m, 1H), 1.72-1.61 (m, 1H). |
| | (±)-3-Oxo-N-(5- | | |
| | (trifluoromethyl)isoxazole-3-yl)- | | |
| | 3,5,6,7,8,9-hexahydro-2H-6,9- | | |
| | epiminocyclohepta- | | |
| | [c]pyridazine-10-carboxamide | | |
| 51^{(e)} | 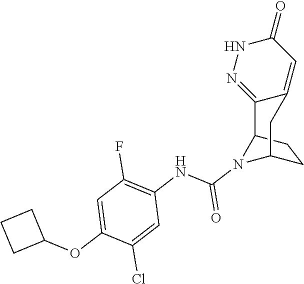 [SMILES: O=C(Nc1cc(Cl)c(OC2CCC2)cc1F)N1C2CCC1c1n[nH]c(=O)cc1C2] | Method 1 419.0, 421.3 at 1.21 min | ¹H NMR (400 MHz, DMSO-d6) δ 12.70 (s, 1H), 8.54 (s, 1H), 7.43 (d, J = 8.1 Hz, 1H), 6.91 (d, J = 11.9 Hz, 1H), 6.67 (s, 1H), 5.00 (d, J = 5.8 Hz, 1H), 4.74 (p, J = 7.1 Hz, 1H), 4.64-4.51 (m, 1H), 3.25-3.10 (m, 1H), 2.62 (d, J = 18.2 Hz, 1H), 2.49-2.37 (m, 2H), 2.25-2.10 (m, 2H), 2.10-1.96 (m, 2H), 1.87-1.71 (m, 2H), 1.71-1.51 (m, 2H). |
| | (±)-N-(5-Chloro-4-cyclobutoxy- | | |
| | 2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 52^{(e)} | 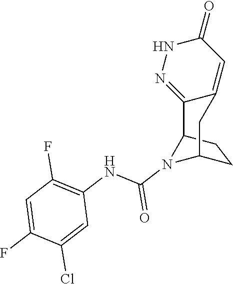 [SMILES: O=C(Nc1cc(Cl)c(F)cc1F)N1C2CCC1c1n[nH]c(=O)cc1C2] | Method 1 365.2, 367.1 at 0.98 min^{(c)} | ¹H NMR (400 MHz, DMSO-d6) δ 12.71 (s, 1H), 8.76 (s, 1H), 7.69 (t, J = 8.0 Hz, 1H), 7.54 (dd, J = 10.4, 9.4 Hz, 1H), 6.68 (s, 1H), 5.03 (d, J = 5.9 Hz, 1H), 4.65-4.52 (m, 1H), 3.24-3.12 (m, 1H), 2.63 (d, J = 18.2 Hz, 1H), 2.28-2.09 (m, 2H), 1.86- 1.73 (m, 1H), 1.73-1.58 (m, 1H). |
| | (±)-N-(5-Chloro-2,4- | | |
| | difluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 53 | 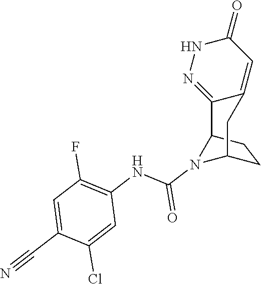 [SMILES: N#Cc1cc(F)c(NC(=O)N2C3CCC2c2n[nH]c(=O)cc2C3)cc1Cl] | Method 1 372.2 at 0.95 min^{(c)} | ¹H NMR (400 MHz, DMSO-d6) δ 12.73 (s, 1H), 9.21 (s, 1H), 8.07 (d, J = 6.8 Hz, 1H), 8.00 (d, J = 10.7 Hz, 1H), 6.67 (s, 1H), 5.10 (d, J = 5.5 Hz, 1H), 4.73- 4.59 (m, 1H), 3.24-3.12 (m, 1H), 2.65 (d, J = 18.4 Hz, 1H), 2.30-2.10 (m, 2H), 1.87-1.75 (m, 1H), 1.74-1.61 (m, 1H) |
| | (±)-N-(5-Chloro-4-cyano-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 74^{(e)} | 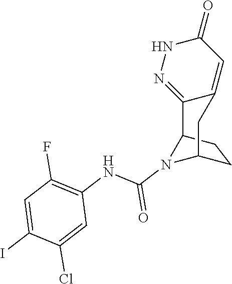 [SMILES: O=C(Nc1cc(Cl)c(I)cc1F)N1C2CCC1c1n[nH]c(=O)cc1C2] | Method 1 474.6, 476.9 at 1.09 min | ¹H NMR (400 MHz, DMSO-d6) δ 12.71 (d, J = 2.1 Hz, 1H), 8.83 (s, 1H), 7.91-7.70 (m, 2H), 6.67 (s, 1H), 5.05 (d, J = 5.8 Hz, 1H), 4.70-4.53 (m, 1H), 3.21- 3.10 (m, 1H), 2.63 (d, J = 18.2 Hz, 1H), 2.26-2.08 (m, 2H), 1.83-1.74 (m, 1H), 1.72-1.61 (m, 1H). |
| | (±)-N-(5-Chloro-2-fluoro-4- | | |
| | iodophenyl)-3-oxo-3,5,6,7,8,9- | | |
| | hexahydro-2H-6,9- | | |
| | epiminocyclohepta[c]pyridazine- | | |
| | 10-carboxamide | | |
| 75^{(e)} | 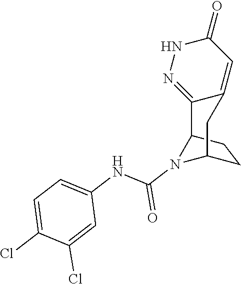 [SMILES: O=C(Nc1ccc(Cl)c(Cl)c1)N1C2CCC1c1n[nH]c(=O)cc1C2] | Method 1 365.3, 367.2 at 1.06 min | ¹H NMR (400 MHz, DMSO-d6) δ 12.73 (s, 1H), 9.04 (s, 1H), 7.85 (d, J = 2.2 Hz, 1H), 7.53- 7.40 (m, 2H), 6.67 (s, 1H), 5.10 (d, J = 5.9 Hz, 1H), 4.71-4.58 (m, 1H), 3.21-3.07 (m, 1H), 2.65 (d, J = 18.1 Hz, 1H), 2.29- 2.06 (m, 2H), 1.86-1.74 (m, 1H), 1.73-1.61 (m, 1H). |
| | (±)-N-(3,4-Dichlorophenyl)-3- | | |
| | oxo-3,5,6,7,8,9-hexahydro-2H- | | |
| | 6,9-epiminocyclohepta- | | |
| | [c]pyridazine-10-carboxamide | | |
| 76^{(e)} | 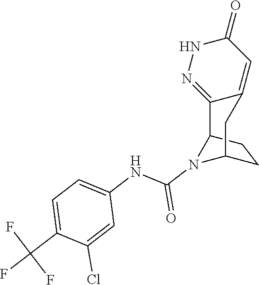 [SMILES: O=C(Nc1ccc(C(F)(F)F)c(Cl)c1)N1C2CCC1c1n[nH]c(=O)cc1C2] | Method 1 397.2, 399.2 at 1.17 min^{(c)} | ¹H NMR (400 MHz, DMSO-d6) δ 12.74 (s, 1H), 9.30 (s, 1H), 7.92 (d, J = 2.0 Hz, 1H), 7.71 (d, J = 8.8 Hz, 1H), 7.65-7.57 (m, 1H), 6.67 (s, 1H), 5.14 (d, J = 5.9 Hz, 1H), 4.73-4.60 (m, 1H), 3.15 (dd, J = 17.9, 5.2 Hz, 1H), 2.66 (d, J = 18.2 Hz, 1H), 2.29- 2.07 (m, 2H), 1.86-1.75 (m, 1H), 1.74-1.62 (m, 1H). |
| | (±)-N-(3-Chloro-4- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 83^{(f)} | 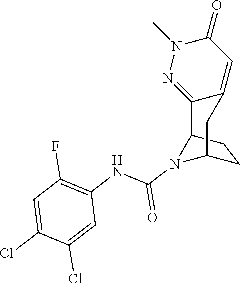 [SMILES: Cn1nc2c(cc1=O)CC1CCC2N1C(=O)Nc1cc(Cl)c(Cl)cc1F] | Method 1 397.2, 399.2 at 1.28 min | ¹H NMR (400 MHz, DMSO-d6) δ 8.88 (s, 1H), 7.82 (d, J = 7.6 Hz, 1H), 7.68 (d, J = 10.3 Hz, 1H), 6.75 (s, 1H), 5.06 (d, J = 5.9 Hz, 1H), 4.73-4.56 (m, 1H), 3.58 (s, 3H), 3.24-3.12 (m, 1H), 2.65 (d, J = 17.9 Hz, 1H), 2.29-2.10 (m, 2H), 1.89-1.74 (m, 1H), 1.75-1.61 (m, 1H). |
| | (±)-N-(4,5-Dichloro-2- | | |
| | fluorophenyl)-2-methyl-3-oxo- | | |
| | 3,5,6,7,8,9-hexahydro-2H-6,9- | | |
| | epiminocyclohepta[c]pyridazine- | | |
| | 10-carboxamide | | |
| 108^{(d)(g)} | 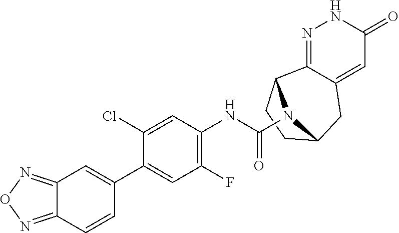 [SMILES: O=C(Nc1cc(Cl)c(-c2ccc3nonc3c2)cc1F)N1[C@H]2CC[C@@H]1c1n[nH]c(=O)cc1C2] | Method 1 465.3, 467.3 at 1.23 min^{(c)} | ¹H NMR (400 MHz, DMSO-d6) δ 12.73 (s, 1H), 8.97 (s, 1H), 8.16-8.09 (m, 2H), 7.86 (d, J = 7.2 Hz, 1H), 7.73-7.66 (m, 1H), 7.54 (d, J = 11.0 Hz, 1H), 6.69 (s, 1H), 5.10 (d, J = 5.8 Hz, 1H), 4.70-4.64 (m, 1H), 3.25-3.16 (m, 1H), 2.66 (d, J = 18.0 Hz, 1H), 2.21 (d, J = 6.8 Hz, 2H), 1.81 (t, J = 9.5 Hz, 1H), 1.70 (d, J = 11.8 Hz, 1H). |
| | (6S,9R)-N-(4-(Benzo[c][1,2,5]- | | |
| | oxadiazol-5-yl)-5-chloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 109^{(d)(g)} | 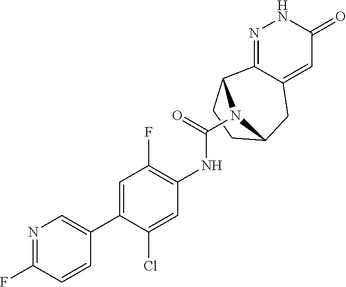 [SMILES: O=C(Nc1cc(Cl)c(-c2ccc(F)nc2)cc1F)N1[C@H]2CC[C@@H]1c1n[nH]c(=O)cc1C2] | Method 1 444.2, 446.2 at 1.04 min | ¹H NMR (400 MHz, DMSO-d6) δ 12.72 (d, J = 2.1 Hz, 1H), 8.92 (s, 1H), 8.31 (s, 1H), 8.10 (td, J = 8.2, 2.6 Hz, 1H), 7.81 (d, J = 7.3 Hz, 1H), 7.46 (d, J = 11.1 Hz, 1H), 7.30 (dd, J = 8.5, 2.8 Hz, 1H), 6.69 (d, J = 2.1 Hz, 1H), 5.08 (d, J = 5.7 Hz, 1H), 4.66-4.62 (m, 1H), 3.20 (dd, J = 18.4, 5.2 Hz, 1H), 2.66 (d, J = 18.1 Hz, 1H), 2.26-2.14 (m, 2H), 1.85-1.76 (m, 1H), 1.73-1.64 (m, 1H). |
| | (6S,9R)-N-(5-Chloro-2-fluoro-4- | | |
| | (6-fluoropyridin-3-yl)phenyl)-3- | | |
| | oxo-3,5,6,7,8,9-hexahydro-2H- | | |
| | 6,9- | | |
| | epiminocyclohepta[c]pyridazine- | | |
| | 10-carboxamide | | |
| 110^{(d)(h)} | 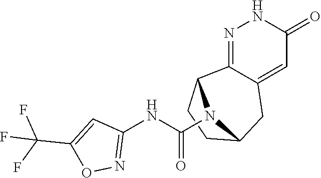 [SMILES: O=C(Nc1cc(C(F)(F)F)on1)N1[C@H]2CC[C@@H]1c1n[nH]c(=O)cc1C2] | Method 1 354.5 at 0.89 min^{(c)} | ¹H NMR (400 MHz, DMSO-d6) δ 12.74 (s, 1H), 10.50 (s, 1H), 7.47 (d, J = 1.0 Hz, 1H), 6.68 (s, 1H), 5.16 (d, J = 4.7 Hz, 1H), 4.70 (t, J = 6.1 H, 1H), 3.13 (dd, J = 18.3, 5.3 Hz, 1H), 2.65 (d, J = 18.1 Hz, 1H), 2.26-2.08 (m, 2H), 1.87-1.75 (m, 1H), 1.73-1.60 (m, 1H). |
| | (6S,9R)-3-Oxo-N-(5- | | |
| | (trifluoromethyl)isoxazol-3-yl)- | | |
| | 3,5,6,7,8,9-hexahydro-2H-6,9- | | |
| | epiminocyclohepta[c]pyridazine- | | |
| | 10-carboxamide | | |
| 111^{(d)(e)} | 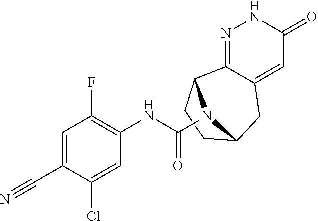 [SMILES: N#Cc1cc(F)c(NC(=O)N2[C@H]3CC[C@@H]2c2n[nH]c(=O)cc2C3)cc1Cl] | Method 1 372.2, 374.2 at 0.9 min | ¹H NMR (400 MHz, DMSO-d6) δ 12.73 (d, J = 1.9 Hz, 1H), 9.21 (s, 1H), 8.07 (d, J = 6.9 Hz, 1H), 8.00 (d, J = 10.6 Hz, 1H), 6.67 (s, 1H), 5.10 (d, J = 5.6 Hz, 1H), 4.66 (t, J = 6.3 Hz, 1H), 3.17 (dd, J = 18.1, 5.3 Hz, 1H), 2.65 (d, J = 18.3 Hz, 1H), 2.26-2.11 (m, 2H), 1.84-1.73 (m, 1H), 1.72- 1.59 (m, 1H). |
| | (6S,9R)-N-(5-Chloro-4-cyano-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 112^{(d)(g)} | 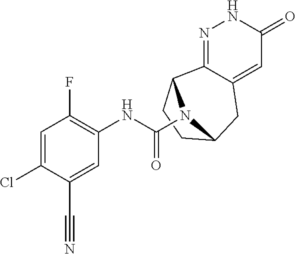 [SMILES: N#Cc1cc(NC(=O)N2[C@H]3CC[C@@H]2c2n[nH]c(=O)cc2C3)c(F)cc1Cl] | Method 1 374.2, 376.2 at 0.87 min | ¹H NMR (400 MHz, DMSO-d6) δ 12.72 (s, 1H), 9.01 (s, 1H), 8.13 (d, J = 7.9 Hz, 1H), 7.83 (d, J = 10.4 Hz, 1H), 6.68 (s, 1H), 5.06 (d, J = 5.7 Hz, 1H), 4.62 (t, J = 5.5 Hz, 1H), 3.22- 3.12 (m, 1H), 2.65 (d, J = 18.3 Hz, 1H), 2.17 (m, 2H), 1.82- 1.76 (m, 1H), 1.74-1.64 (m, 1H). |
| | (6S,9R)-N-(4-Chloro-5-cyano-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 113^{(d)(h)} | 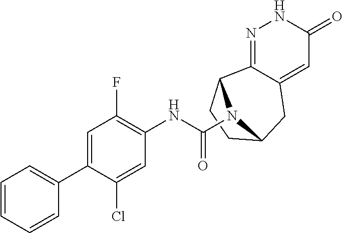 [SMILES: O=C(Nc1cc(Cl)c(-c2ccccc2)cc1F)N1[C@H]2CC[C@@H]1c1n[nH]c(=O)cc1C2] | Method 1 425.2, 427.2 at 1.32 min | ¹H NMR (400 MHz, DMSO-d6) δ 12.74 (s, 1H), 8.86 (s, 1H), 7.75 (d, J = 7.3 Hz, 1H), 7.51- 7.36 (m, 5H), 7.30 (d, J = 11.1 Hz, 1H), 6.69 (s, 1H), 5.08 (d, J = 5.8 Hz, 1H), 4.64 (s, 1H), 3.20 (dd, J = 18.1, 5.0 Hz, 1H), 2.63 (s, 1H), 2.23-2.14 (m, 2H), 1.79 (d, J = 10.5 Hz, 1H), 1.69 (d, J = 11.1 Hz, 1H) |
| | (6S,9R)-N-(2-Chloro-5-fluoro- | | |
| | [1,1′-biphenyl]-4-yl)-3-oxo- | | |
| | 3,5,6,7,8,9-hexahydro-2H-6,9- | | |
| | epiminocyclohepta[c]pyridazine- | | |
| | 10-carboxamide | | |
| 119^{(d)(i)} | 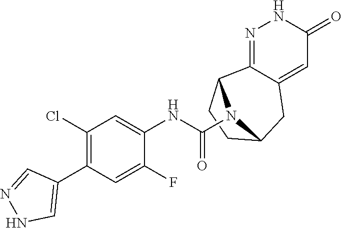 [SMILES: O=C(Nc1cc(Cl)c(-c2cn[nH]c2)cc1F)N1[C@H]2CC[C@@H]1c1n[nH]c(=O)cc1C2] | Method 1 415.3, 417.3 at 0.76 min | ¹H NMR (400 MHz, DMSO-d6) δ 12.72 (s, 1H), 8.77 (s, 1H), 8.37 (s, 1H), 7.66 (d, J = 7.5 Hz, 1H), 7.51 (d, J = 11.9 Hz, 1H), 6.68 (s, 1H), 5.05 (s, 1H), 4.63 (s, 1H), 3.17 (s, 1H), 2.53 (s, 1H), 2.19 (d, J = 6.5 Hz, 2H), 1.85-1.75 (m, 1H), 1.71-1.45 (m, 1H), (2 exchangeable protons not observed) |
| | (6S,9R)-N-(5-Chloro-2-fluoro-4- | | |
| | (1H-pyrazol-4-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 120^{(d)(j)} | 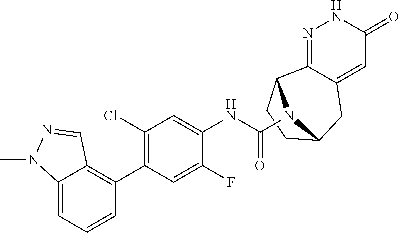 [SMILES: Cn1ncc2c(-c3cc(F)c(NC(=O)N4[C@H]5CC[C@@H]4c4n[nH]c(=O)cc4C5)cc3Cl)cccc21] | Method 1 479.2, 481.2 at 1.14 min | ¹H NMR (400 MHz, DMSO-d6) δ 12.73 (s, 1H), 8.92 (s, 1H), 7.84 (d, J = 7.3 Hz, 1H), 7.76 (d, J = 1.0 Hz, 1H), 7.70 (d, J = 8.5 Hz, 1H), 7.48 (dd, J = 8.5, 7.1 Hz, 1H), 7.37 (d, J = 11.0 Hz, 1H), 7.11 (d, J = 6.8 Hz, 1H), 6.70 (s, 1H), 5.10 (d, J = 5.8 Hz, 1H), 4.66 (s, 1H), 4.08 (s, 3H), 3.22 (d, J = 19.5 Hz, 1H), 2.66 (d, J = 18.3 Hz, 1H), 2.21 (s, 2H), 1.81 (t, J = 9.4 Hz, 1H), 1.70 (d, J = 12.0 Hz, 1H). |
| | (6S,9R)-N-(5-Chloro-2-fluoro-4- | | |
| | (1-methyl-1H-indazol-4- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 152^{(d)(j)} | 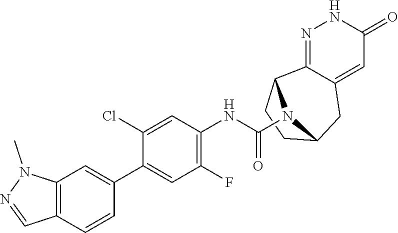 [SMILES: Cn1ncc2ccc(-c3cc(F)c(NC(=O)N4[C@H]5CC[C@@H]4c4n[nH]c(=O)cc4C5)cc3Cl)cc21] | Method 1 479.2, 481.2 at 1.06 min | ¹H NMR (400 MHz, DMSO-d6) δ 8.10 (d, J = 0.9 Hz, 1H), 7.81- 7.78 (m, 1H), 7.76 (d, J = 7.4 Hz, 1H), 7.70 (d, J = 8.7 Hz, 1H), 7.45 (dd, J = 8.7, 1.7 Hz, 1H), 7.34 (d, J = 11.2 Hz, 1H), 6.69 (s, 1H), 5.08 (d, J = 5.7 Hz, 1H), 4.68-4.62 (m, 1H), 4.08 (s, 3H), 3.25-3.13 (m, 1H), 2.65 (d, J = 18.3 Hz, 1H), 2.25-2.13 (m, 2H), 1.83-1.73 (m, 1H), 1.73-1.66 (m, 1H). |
| | (6S,9R)-N-(5-Chloro-2-fluoro-4- | | |
| | (1-methyl-1H-indazol-6- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 153^{(d)(j)} | 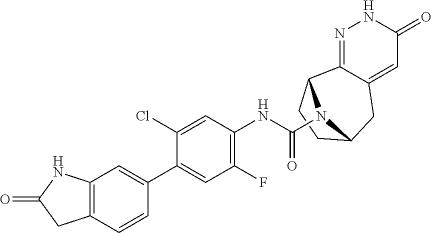 [SMILES: O=C1Cc2ccc(-c3cc(F)c(NC(=O)N4[C@H]5CC[C@@H]4c4n[nH]c(=O)cc4C5)cc3Cl)cc2N1] | Method 1 480.3, 482.2 at 0.89 min | |
| | (6S,9R)-N-(5-Chloro-2-fluoro-4- | | |
| | (2-oxoindolin-6-yl)phenyl)-3- | | |
| | oxo-3,5,6,7,8,9-hexahydro-2H- | | |
| | 6,9- | | |
| | epiminocyclohepta[c]pyridazine- | | |
| | 10-carboxamide | | |
| 163^{(d)(k)} | 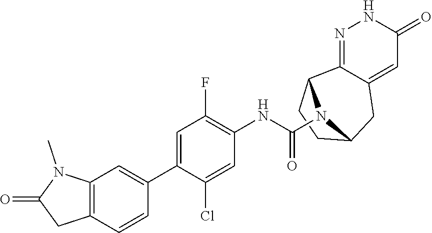 [SMILES: CN1C(=O)Cc2ccc(-c3cc(F)c(NC(=O)N4[C@H]5CC[C@@H]4c4n[nH]c(=O)cc4C5)cc3Cl)cc21] | Method 2 494.1, 496.1 at 1.58 min | ¹H NMR (400 MHz, DMSO-d6) δ 12.72 (s, 1H), 8.86 (s, 1H), 7.75 (d, J = 7.3 Hz, 1H), 7.36- 7.29 (m, 2H), 7.09-7.00 (m, 2H), 6.69 (s, 1H), 5.08 (d, J = 5.7 Hz, 1H), 4.65 (t, J = 5.6 Hz, 1H), 3.59 (s, 2H), 3.25-3.16 (m, 1H), 3.13 (s, 3H), 2.66 (d, J = 18.2 Hz, 1H), 2.28-2.12 (m, 2H), 1.80 (t, J = 9.6 Hz, 1H), 1.73-1.64 (m, 1H). |
| | (6S,9R)-N-(5-Chloro-2-fluoro-4- | | |
| | (1-methyl-2-oxoindolin-6- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 164^{(d)(k)} | 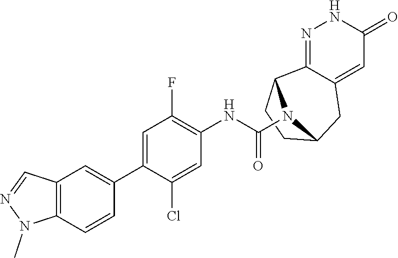 [SMILES: Cn1ncc2cc(-c3cc(F)c(NC(=O)N4[C@H]5CC[C@@H]4c4n[nH]c(=O)cc4C5)cc3Cl)ccc21] | Method 2 479.1, 481.0 at 1.66 min | ¹H NMR (400 MHz, DMSO-d6) δ 12.73 (s, 1H), 8.86 (s, 1H), 8.10 (d, J = 0.9 Hz, 1H), 7.80 (m, 1H), 7.75 (d, J = 7.3 Hz, 1H), 7.70 (dt, J = 8.8, 0.9 Hz, 1H), 7.45 (dd, J = 8.7, 1.6 Hz, 1H), 7.35 (d, J = 11.1 Hz, 1H), 6.69 (m, 1H), 5.08 (d, J = 5.6 Hz, 1H), 4.65 (t, J = 5.9 Hz, 1H), 4.08 (s, 3H), 3.21 (dd, J = 18.3, 5.3 Hz, 1H), 2.66 (d, J = 18.2 Hz, 1H), 2.26-2.12 (m, 2H), 1.88-1.76 (m, 1H), 1.73-1.61 (m, 1H). |
| | (6S,9R)-N-(5-Chloro-2-fluoro-4- | | |
| | (1-methyl-1H-indazol-5- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 165^{(d)(l)} | 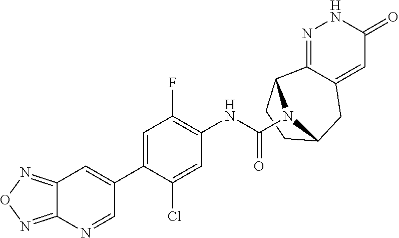 [SMILES: O=C(Nc1cc(Cl)c(-c2cnc3nonc3c2)cc1F)N1[C@H]2CC[C@@H]1c1n[nH]c(=O)cc1C2] | Method 2 468.0, 470.0 at 1.63 min | ¹H NMR (400 MHz, DMSO-d6) δ 12.73 (s, 1H), 9.19 (d, J = 2.1 Hz, 1H), 9.02 (s, 1H), 8.72 (d, J = 2.1 Hz, 1H), 7.91 (d, J = 7.2 Hz, 1H), 7.64 (d, J = 11.0 Hz, 1H), 6.70 (s, 1H), 5.10 (d, J = 5.6 Hz, 1H), 4.67 (t, J = 6.3 Hz, 1H), 3.21 (dd, J = 18.4, 5.4 Hz, 1H), 2.67 (d, J = 18.3 Hz, 1H), 2.27-2.13 (m, 2H), 1.89-1.77 (m, 1H), 1.74-1.62 (m, 1H). |
| | (6S,9R)-N-(4- | | |
| | ([1,2,5]Oxadiazolo[3,4- | | |
| | b]pyridine-6-yl)-5-chloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 166^{(d)(m)} | 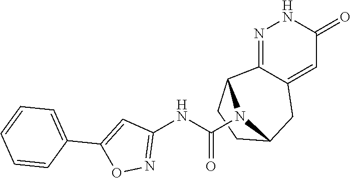 [SMILES: O=C(Nc1cc(-c2ccccc2)on1)N1[C@H]2CC[C@@H]1c1n[nH]c(=O)cc1C2] | Method 2 364.1 at 1.49 min | ¹H NMR (400 MHz, DMSO-d6) δ 12.73 (s, 1H), 10.12 (s, 1H), 7.87-7.79 (m, 2H), 7.56-7.44 (m, 3H), 7.21 (s, 1H), 6.68 (s, 1H), 5.18 (d, J = 5.7 Hz, 1H), 4.72 (d, J = 4.9 Hz, 1H), 3.18- 3.09 (m, 1H), 2.65 (d, J = 18.1 Hz, 1H), 2.26-2.12 (m, 2H), 1.84-1.75 (m, 1H), 1.72-1.65 (m, 1H). |
| | (6S,9R)-3-Oxo-N-(5- | | |
| | phenylisoxazol-3-yl)-3,5,6,7,8,9- | | |
| | hexahydro-2H-6,9- | | |
| | epiminocyclohepta[c]pyridazine- | | |
| | 10-carboxamide | | |
| 168^{(d)(g)} | 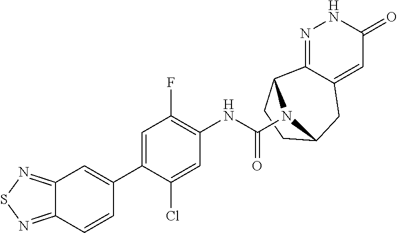 [SMILES: O=C(Nc1cc(Cl)c(-c2ccc3nsnc3c2)cc1F)N1[C@H]2CC[C@@H]1c1n[nH]c(=O)cc1C2] | Method 1 483.0, 485.0 at 1.23 min | ¹H NMR (400 MHz, DMSO-d6) δ 12.73 (s, 1H), 8.95 (s, 1H), 8.19-8.13 (m, 2H), 7.85 (d, J = 7.3 Hz, 1H), 7.83-7.77 (m, 1H), 7.53 (d, J = 11.0 Hz, 1H), 6.70 (s, 1H), 5.10 (d, J = 5.7 Hz, 1H), 4.67 (d, J = 6.2 Hz, 1H), 3.21 (dd, J = 18.2, 5.4 Hz, 1H), 2.66 (d, J = 18.2 Hz, 1H), 2.29-2.12 (m, 2H), 1.81 (t, J = 9.7 Hz, 1H), 1.75-1.63 (m, 1H) |
| | (6S,9R)-N-(4- | | |
| | (Benzo[c][1,2,5]thiadiazol-5-yl)- | | |
| | 5-chloro-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 170^{(d)(l)} | 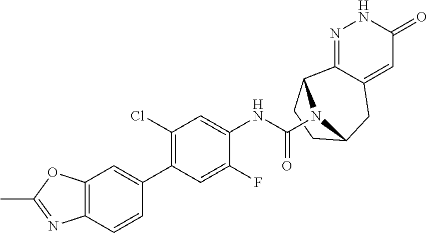 [SMILES: Cc1nc2ccc(-c3cc(F)c(NC(=O)N4[C@H]5CC[C@@H]4c4n[nH]c(=O)cc4C5)cc3Cl)cc2o1] | Method 1 480.3, 482.3 at 1.10 min | ¹H NMR (500 MHz, DMSO-d6) δ 12.72 (s, 1H), 8.88 (s, 1H), 7.79-7.69 (m, 3H), 7.41-7.34 (m, 2H), 6.69 (s, 1H), 5.08 (d, J = 5.8 Hz, 1H), 4.65 (t, J = 6.3 Hz, 1H), 3.20 (dd, J = 18.3, 5.3 Hz, 1H), 2.69-2.60 (m, 4H), 2.28- 2.13 (m, 2H), 1.86-1.77 (m, 1H), 1.73-1.64 (m, 1H). |
| | (6S,9R)-N-(5-Chloro-2-fluoro-4- | | |
| | (2-methylbenzo[d]oxazol-6- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 171^{(d)(j)} | 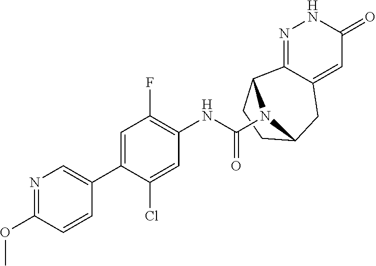 [SMILES: COc1ccc(-c2cc(F)c(NC(=O)N3[C@H]4CC[C@@H]3c3n[nH]c(=O)cc3C4)cc2Cl)cn1] | Method 2 456.3, 458.3 at 1.54 min | ¹H NMR (500 MHz, DMSO-d6) δ 12.72 (s, 1H), 8.90 (s, 1H), 8.23 (dd, J = 2.6, 0.8 Hz, 1H), 7.81 (dd, J = 8.6, 2.5 Hz, 1H), 7.76 (d, J = 7.3 Hz, 1H), 7.37 (d, J = 11.0 Hz, 1H), 6.91 (dd, J = 8.6, 0.8 Hz, 1H), 6.69 (d, J = 1.6 Hz, 1H), 5.08 (d, J = 5.9 Hz, 1H), 4.65 (d, J = 6.3 Hz, 1H), 3.90 (s, 3H), 3.20 (dd, J = 18.4, 5.3 Hz, 1H), 2.65 (d, J = 18.2 Hz, 1H), 2.19 (dd, J = 12.3, 6.4 Hz, 2H), 1.80 (t, J = 9.8 Hz, 1H), 1.69 (d, J = 11.4 Hz, 1H). |
| | (6S,9R)-N-(5-Chloro-2-fluoro-4- | | |
| | (6-methoxypyridin-3-yl)phenyl)- | | |
| | 3-oxo-3,5,6,7,8,9-hexahydro- | | |
| | 2H-6,9- | | |
| | epiminocyclohepta[c]pyridazine- | | |
| | 10-carboxamide | | |
| 173^{(d)(l)} | 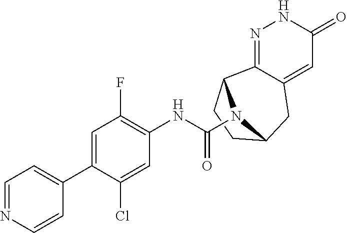 [SMILES: O=C(Nc1cc(Cl)c(-c2ccncc2)cc1F)N1[C@H]2CC[C@@H]1c1n[nH]c(=O)cc1C2] | Method 1 426.3, 428.3 at 0.89 min | ¹H NMR (400 MHz, DMSO-d6) δ 8.63 (dd, J = 2.4, 0.9 Hz, 1H), 8.59 (dd, J = 4.8, 1.6 Hz, 1H), 7.88 (dt, J = 8.0, 2.0 Hz, 1H), 7.85 (d, J = 7.4 Hz, 1H), 7.49 (ddd, J = 7.9, 4.8, 0.9 Hz, 1H), 7.39 (d, J = 11.2 Hz, 1H), 6.67 (s, 1H), 5.08 (d, J = 5.5 Hz, 1H), 4.65 (d, J = 6.2 Hz, 1H), 3.20 (dd, J = 18.5, 5.4 Hz, 1H), 2.64 (d, J = 18.3 Hz, 1H), 2.24-2.10 (m, 2H), 1.83-1.74 (m, 1H), 1.71-1.63 (m, 1H). 2 exchangeable protons not observed |
| | (6S,9R)-N-(5-Chloro-2-fluoro-4- | | |
| | (pyridin-4-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 174^{(d)(l)} | 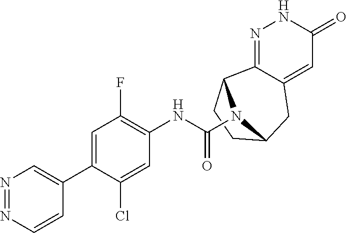 [SMILES: O=C(Nc1cc(Cl)c(-c2ccnnc2)cc1F)N1[C@H]2CC[C@@H]1c1n[nH]c(=O)cc1C2] | Method 1 427.3, 429.3 at 0.72 min | ¹H NMR (400 MHz, DMSO-d6) δ 12.73 (s, 1H), 9.37 (dd, J = 2.5, 1.2 Hz, 1H), 9.34 (dd, J = 5.3, 1.3 Hz, 1H), 8.99 (s, 1H), 7.89 (d, J = 7.2 Hz, 1H), 7.84 (dd, J = 5.3, 2.4 Hz, 1H), 7.58 (d, J = 11.1 Hz, 1H), 6.69 (s, 1H), 5.09 (d, J = 5.5 Hz, 1H), 4.66 (d, J = 6.4 Hz, 1H), 3.29- 3.11 (m, 1H), 2.66 (d, J = 18.2 Hz, 1H), 2.19 (dd, J = 11.6, 6.4 Hz, 2H), 1.87-1.75 (m, 1H), 1.68 (dd, J = 10.1, 6.2 Hz, 1H). |
| | (6S,9R)-N-(5-Chloro-2-fluoro-4- | | |
| | (pyridazin-4-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 175^{(d)(l)} | 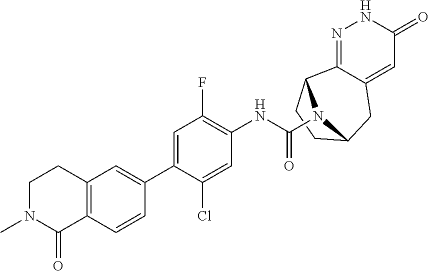 [SMILES: CN1CCc2cc(-c3cc(F)c(NC(=O)N4[C@H]5CC[C@@H]4c4n[nH]c(=O)cc4C5)cc3Cl)ccc2C1=O] | Method 1 508.5, 510.6 at 1.01 min | ¹H NMR (400 MHz, DMSO-d6) δ 12.72 (s, 1H), 8.89 (s, 1H), 7.92 (d, J = 8.0 Hz, 1H), 7.78 (d, J = 7.3 Hz, 1H), 7.40 (dd, J = 8.0, 1.8 Hz, 1H), 7.35 (d, J = 11.2 Hz, 2H), 6.69 (d, J = 1.5 Hz, 1H), 5.08 (d, J = 5.6 Hz, 1H), 4.65 (d, J = 6.0 Hz, 1H), 3.58 (t, J = 6.7 Hz, 2H), 3.32 (s, 4H), 3.26-3.14 (m, 1H), 3.02 (d, J = 6.6 Hz, 1H), 2.65 (d, J = 18.2 Hz, 1H), 2.29-2.10 (m, 2H), 1.86-1.75 (m, 1H), 1.73- 1.62 (m, 1H). |
| | (6S,9R)-N-(5-Chloro-2-fluoro-4- | | |
| | (2-methyl-1-oxo-1,2,3,4- | | |
| | tetrahydroisoquinolin-6- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 178^{(d)(g)} | 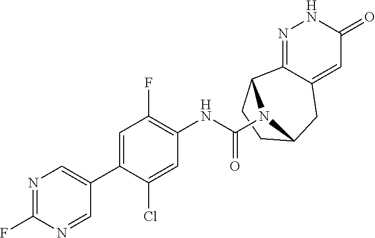 [SMILES: O=C(Nc1cc(Cl)c(-c2cnc(F)nc2)cc1F)N1[C@H]2CC[C@@H]1c1n[nH]c(=O)cc1C2] | Method 2 445.3, 447.3 at 1.38 min | ¹H NMR (400 MHz, DMSO-d6) δ 12.73 (s, 1H), 8.96 (s, 1H), 8.94 (d, J = 1.5 Hz, 2H), 7.86 (d, J = 7.2 Hz, 1H), 7.57 (d, J = 11.0 Hz, 1H), 6.69 (s, 1H), 5.09 (d, J = 5.6 Hz, 1H), 4.65 (s, 1H), 3.20 (d, J = 14.2 Hz, 1H), 2.66 (d, J = 18.0 Hz, 1H), 2.20 (d, J = 6.4 Hz, 2H), 1.79 (d, J = 10.8 Hz, 1H), 1.68 (s, 1H). |
| | (6S,9R)-N-(5-Chloro-2-fluoro-4- | | |
| | (2-fluoropyrimidin-5-yl)phenyl)- | | |
| | 3-oxo-3,5,6,7,8,9-hexahydro- | | |
| | 2H-6,9- | | |
| | epiminocyclohepta[c]pyridazine- | | |
| | 10-carboxamide | | |
| 179^{(d)(g)} | 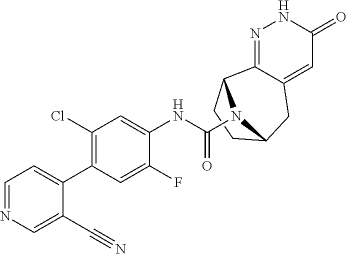 [SMILES: N#Cc1cnccc1-c1cc(F)c(NC(=O)N2[C@H]3CC[C@@H]2c2n[nH]c(=O)cc2C3)cc1Cl] | Method 1 449.2, 451.1 at 0.88 min | ¹H NMR (400 MHz, DMSO-d6) δ 12.73 (s, 1H), 9.16 (d, J = 0.8 Hz, 1H), 9.00 (s, 1H), 8.95 (d, J = 5.2 Hz, 1H), 7.91 (d, J = 7.1 Hz, 1H), 7.66 (dd, J = 5.2, 0.8 Hz, 1H), 7.56 (d, J = 10.8 Hz, 1H), 6.69 (s, 1H), 5.09 (d, J = 5.7 Hz, 1H), 4.67 (d, J = 6.1 Hz, 1H), 3.26-3.14 (m, 1H), 2.66 (d, J = 18.2 Hz, 1H), 2.20 (dd, J = 11.9, 6.3 Hz, 2H), 1.81 (t, J = 9.6 Hz, 1H), 1.75-1.64 (m, 1H). |
| | (6S,9R)-N-(5-Chloro-4-(3- | | |
| | cyanopyridin-4-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 180^{(d)(g)} | 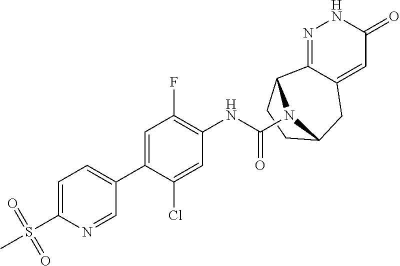 [SMILES: CS(=O)(=O)c1ccc(-c2cc(F)c(NC(=O)N3[C@H]4CC[C@@H]3c3n[nH]c(=O)cc3C4)cc2Cl)cn1] | Method 1 503.8, 506.5 at 0.86 min | ¹H NMR (400 MHz, DMSO-d6) δ 12.76-12.69 (m, 1H), 8.98 (s, 1H), 8.87 (dd, J = 2.2, 0.8 Hz, 1H), 8.27 (dd, J = 8.2, 2.2 Hz, 1H), 8.14 (dd, J = 8.1, 0.8 Hz, 1H), 7.88 (d, J = 7.3 Hz, 1H), 7.55 (d, J = 11.0 Hz, 1H), 6.69 (s, 1H), 5.09 (d, J = 5.8 Hz, 1H), 4.66 (d, J = 6.1 Hz, 1H), 3.35 (s, 3H), 3.24-3.16 (m, 1H), 2.66 (d, J = 18.1 Hz, 1H), 2.19 (dd, J = 11.9, 6.2 Hz, 2H), 1.81 (t, J = 9.6 Hz, 1H), 1.69 (t, J = 8.6 Hz, 1H). |
| | (6S,9R)-N-(5-Chloro-2-fluoro-4- | | |
| | (6-(methylsulfonyl)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 181^{(d)(l)} | 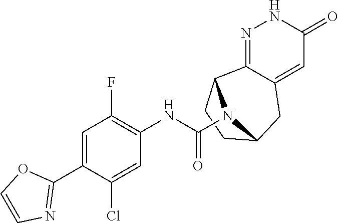 [SMILES: O=C(Nc1cc(Cl)c(-c2ncco2)cc1F)N1[C@H]2CC[C@@H]1c1n[nH]c(=O)cc1C2] | Method 1 416.6, 418.3 at 1.30 min | ¹H NMR (400 MHz, DMSO-d6) δ 12.72 (s, 1H), 9.02 (s, 1H), 8.30 (s, 1H), 7.93 (d, J = 7.2 Hz, 1H), 7.79 (d, J = 11.3 Hz, 1H), 7.47-7.42 (m, 1H), 6.68 (s, 1H), 5.09 (d, J = 5.6 Hz, 1H), 4.69-4.62 (m, 1H), 3.23-3.15 (m, 1H), 2.70-2.61 (m, 1H), 2.23-2.14 (m, 2H), 1.79 (d, J = 11.1 Hz, 1H), 1.69 (d, J = 11.5 Hz, 1H). |
| | (6S,9R)-N-(5-Chloro-2-fluoro-4- | | |
| | (oxazol-2-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 182^{(d)(l)} | 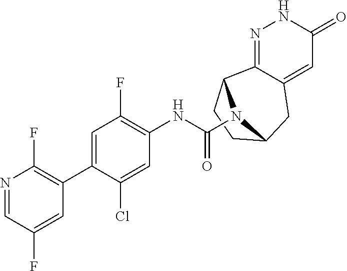 [SMILES: O=C(Nc1cc(Cl)c(-c2cc(F)cnc2F)cc1F)N1[C@H]2CC[C@@H]1c1n[nH]c(=O)cc1C2] | Method 2 462.3, 464.0 at 1.53 min | ¹H NMR (400 MHz, DMSO-d6) δ 12.73 (s, 1H), 8.96 (s, 1H), 8.35 (dd, J = 3.0, 1.7 Hz, 1H), 8.10 (td, J = 7.6, 3.0 Hz, 1H), 7.84 (d, J = 7.2 Hz, 1H), 7.51 (d, J = 10.9 Hz, 1H), 6.69 (s, 1H), 5.08 (d, J = 5.7 Hz, 1H), 4.65 (s, 1H), 3.20 (d, J = 16.4 Hz, 1H), 2.66 (d, J = 18.1 Hz, 1H), 2.29- 2.11 (m, 2H), 1.80 (t, J = 9.7 Hz, 1H), 1.68 (t, J = 9.0 Hz, 1H). |
| | (6S,9R)-N-(5-Chloro-4-(2,5- | | |
| | difluoropyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 188^{(d)(l)} | 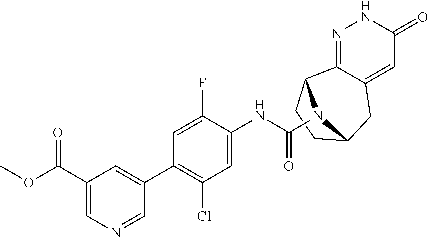 [SMILES: COC(=O)c1cncc(-c2cc(F)c(NC(=O)N3[C@H]4CC[C@@H]3c3n[nH]c(=O)cc3C4)cc2Cl)c1] | Method 1 484.3, 486.3 at 1.00 min | ¹H NMR (400 MHz, DMSO-d6) δ 12.73 (s, 1H), 9.11 (d, J = 2.0 Hz, 1H), 8.95 (s, 1H), 8.89 (d, J = 2.3 Hz, 1H), 8.34 (t, J = 2.1 Hz, 1H), 7.85 (d, J = 7.3 Hz, 1H), 7.53 (d, J = 11.1 Hz, 1H), 6.69 (s, 1H), 5.09 (d, J = 5.7 Hz, 1H), 4.66 (d, J = 6.2 Hz, 1H), 3.91 (s, 3H), 3.20 (dd, J = 18.6, 5.3 Hz, 1H), 2.66 (d, J = 18.2 Hz, 1H), 2.34-2.10 (m, 2H), 1.90-1.76 (m, 1H), 1.69 (t, J = 8.7 Hz, 1H). |
| | Methyl 5-(2-chloro-5-fluoro-4- | | |
| | ((6S,9R)-3-oxo-3,5,6,7,8,9- | | |
| | hexahydro-2H-6,9- | | |
| | epiminocyclohepta[c]pyridazine- | | |
| | 10- | | |
| | carboxamido)phenyl)nicotinate | | |
| 191^{(d)(j)} | 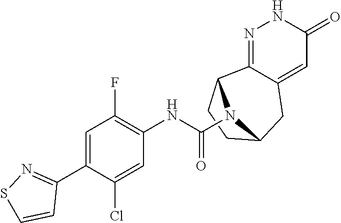 [SMILES: O=C(Nc1cc(Cl)c(-c2ccsn2)cc1F)N1[C@H]2CC[C@@H]1c1n[nH]c(=O)cc1C2] | Method 2 432.6, 434.4 at 1.45 min | ¹H NMR (400 MHz, DMSO-d6) δ 12.72 (s, 1H), 9.19 (d, J = 4.7 Hz, 1H), 8.94 (s, 1H), 7.83 (d, J = 7.2 Hz, 1H), 7.78 (d, J = 4.7 Hz, 1H), 7.60 (d, J = 11.3 Hz, 1H), 6.69 (d, J = 2.1 Hz, 1H), 5.09 (d, J = 5.7 Hz, 1H), 4.66 (d, J = 6.4 Hz, 1H), 3.20 (dd, J = 17.9, 5.7 Hz, 1H), 2.63 (s, 1H), 2.19 (dd, J = 11.6, 6.4 Hz, 2H), 1.80 (t, J = 9.6 Hz, 1H), 1.68 (t, J = 8.2 Hz, 1H). |
| | (6S,9R)-N-(5-Chloro-2-fluoro-4- | | |
| | (isothiazol-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 192^{(d)(j)} | 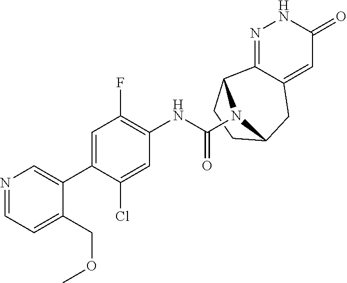 [SMILES: COCc1ccncc1-c1cc(F)c(NC(=O)N2[C@H]3CC[C@@H]2c2n[nH]c(=O)cc2C3)cc1Cl] | Method 2 470.1, 472.2 at 0.83 min | ¹H NMR (400 MHz, DMSO-d6) δ 8.92 (s, 1H), 8.61 (d, J = 5.1 Hz, 1H), 8.34 (s, 1H), 8.28 (s, 1H), 7.82 (dd, J = 7.2, 4.0 Hz, 1H), 7.51 (d, J = 5.1 Hz, 1H), 7.35 (d, J = 10.8 Hz, 1H), 6.69 (s, 1H), 5.09 (t, J = 7.5 Hz, 1H), 4.65 (d, J = 6.4 Hz, 1H), 4.23 (dd, J = 14.2, 4.3 Hz, 2H), 3.24 (s, 3H), 3.20-3.17 (m, 1H), 2.66 (d, J = 18.2 Hz, 1H), 2.21 (p, J = 7.4 Hz, 2H), 1.81 (t, J = 9.4 Hz, 1H), 1.69 (t, J = 8.5 Hz, 1H). |
| | (6S,9R)-N-(5-Chloro-2-fluoro-4- | | |
| | (4-(methoxymethyl)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 195^{(d)(j)} | 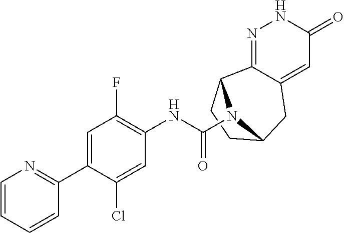 [SMILES: O=C(Nc1cc(Cl)c(-c2ccccn2)cc1F)N1[C@H]2CC[C@@H]1c1n[nH]c(=O)cc1C2] | Method 1 426.2, 428.3 at 0.95 min | ¹H NMR (400 MHz, DMSO-d6) δ 12.72 (s, 1H), 8.91 (s, 1H), 8.68 (ddd, J = 4.9, 1.8, 1.0 Hz, 1H), 7.90 (td, J = 7.7, 1.8 Hz, 1H), 7.79 (d, J = 7.2 Hz, 1H), 7.70 (dt, J = 7.9, 1.1 Hz, 1H), 7.46 (d, J = 11.2 Hz, 1H), 7.42 (ddd, J = 7.6, 4.8, 1.1 Hz, 1H), 6.69 (d, J = 1.8 Hz, 1H), 5.09 (d, J = 5.8 Hz, 1H), 4.65 (t, J = 5.9 Hz, 1H), 3.20 (dd, J = 18.7, 5.4 Hz, 2H), 2.73-2.56 (m, 1H), 2.29-2.11 (m, 2H), 1.85-1.75 (m, 1H), 1.75-1.64 (m, 1H). |
| | (6S,9R)-N-(5-Chloro-2-fluoro-4- | | |
| | (pyridin-2-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 196^{(d)(j)} | 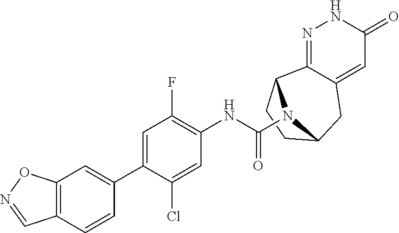 [SMILES: O=C(Nc1cc(Cl)c(-c2ccc3cnoc3c2)cc1F)N1[C@H]2CC[C@@H]1c1n[nH]c(=O)cc1C2] | Method 2 466.1, 468.4 at 1.48 min | ¹H NMR (400 MHz, DMSO-d6) δ 12.72 (s, 1H), 8.91 (s, 1H), 7.78 (d, J = 7.2 Hz, 1H), 7.66 (d, J = 8.0 Hz, 1H), 7.33 (d, J = 11.0 Hz, 1H), 7.01 (d, J = 1.5 Hz, 1H), 6.93 (dd, J = 8.1, 1.6 Hz, 1H), 6.68 (d, J = 1.7 Hz, 1H), 5.07 (d, J = 5.6 Hz, 1H), 4.64 (t, J = 6.1 Hz, 1H), 3.23-3.11 (m, 1H), 2.74-2.58 (m, 1H), 2.29-2.10 (m, 2H), 1.87-1.74 (m, 1H), 1.74-1.62 (m, 1H). |
| | (6S,9R)-N-(4-(Benzo[d]isoxazol- | | |
| | 6-yl)-5-chloro-2-fluorophenyl)-3- | | |
| | oxo-3,5,6,7,8,9-hexahydro-2H- | | |
| | 6,9-epiminocyclohepta- | | |
| | [c]pyridazine-10-carboxamide | | |
| 197^{(d)(j)} | 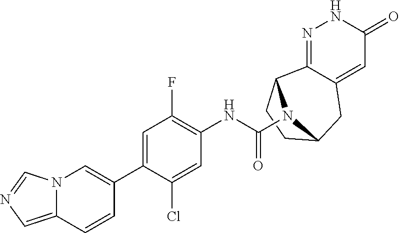 [SMILES: O=C(Nc1cc(Cl)c(-c2ccc3cncn3c2)cc1F)N1[C@H]2CC[C@@H]1c1n[nH]c(=O)cc1C2] | Method 2 464.9, 467.4 at 0.96 min | ¹H NMR (400 MHz, DMSO-d6) δ 8.46 (q, J = 1.2 Hz, 1H), 8.41 (d, J = 0.9 Hz, 1H), 7.81 (d, J = 7.3 Hz, 1H), 7.61 (d, J = 9.3 Hz, 1H), 7.44 (d, J = 11.1 Hz, 1H), 7.41 (s, 1H), 6.85 (dd, J = 9.3, 1.5 Hz, 1H), 6.69 (s, 1H), 5.09 (d, J = 5.6 Hz, 1H), 4.66 (s, 1H), 3.22 (d, J = 19.2 Hz, 1H), 2.75- 2.62 (m, 1H), 2.21 (q, J = 7.2 Hz, 2H), 1.81 (t, J = 9.7 Hz, 1H), 1.70 (d, J = 13.3 Hz, 1H). |
| | (6S,9R)-N-(5-Chloro-2-fluoro-4- | | |
| | (imidazo[1,5-a]pyridin-6- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 198^{(d)(j)} | 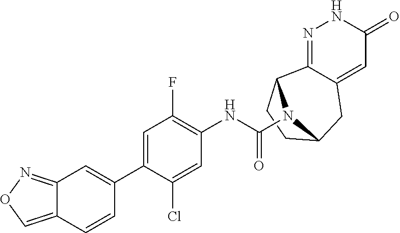 [SMILES: O=C(Nc1cc(Cl)c(-c2ccc3conc3c2)cc1F)N1[C@H]2CC[C@@H]1c1n[nH]c(=O)cc1C2] | Method 1 466.6, 468.6 at 1.11 min | ¹H NMR (400 MHz, DMSO-d6) δ 12.73 (s, 1H), 9.85 (d, J = 1.1 Hz, 1H), 8.92 (s, 1H), 7.81 (d, J = 7.7 Hz, 2H), 7.68 (d, J = 1.3 Hz, 1H), 7.46 (d, J = 11.0 Hz, 1H), 7.13 (dd, J = 9.0, 1.3 Hz, 1H), 6.69 (s, 1H), 5.09 (d, J = 5.7 Hz, 1H), 4.65 (s, 1H), 3.19 (s, 1H), 2.66 (d, J = 18.1 Hz, 1H), 2.28-2.11 (m, 2H), 1.81 (t, J = 9.6 Hz, 1H), 1.69 (t, J = 8.4 Hz, 1H |
| | (6S,9R)-N-(4-(Benzo[c]isoxazol- | | |
| | 6-yl)-5-chloro-2-fluorophenyl)-3- | | |
| | oxo-3,5,6,7,8,9-hexahydro-2H- | | |
| | 6,9-epiminocyclohepta- | | |
| | [c]pyridazine-10-carboxamide | | |
| 199^{(d)(j)} | 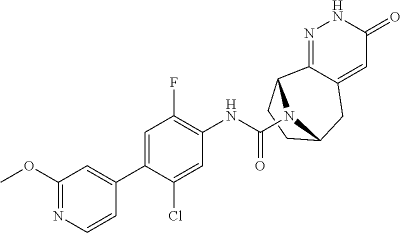 [SMILES: COc1cc(-c2cc(F)c(NC(=O)N3[C@H]4CC[C@@H]3c3n[nH]c(=O)cc3C4)cc2Cl)ccn1] | Method 1 456.2, 458.3 at 1.13 min | ¹H NMR (400 MHz, DMSO-d6) δ 12.72 (d, J = 2.2 Hz, 1H), 8.92 (s, 1H), 8.24 (dd, J = 5.3, 0.7 Hz, 1H), 7.80 (d, J = 7.3 Hz, 1H), 7.39 (d, J = 11.1 Hz, 1H), 7.06 (dd, J = 5.3, 1.5 Hz, 1H), 6.87 (dd, J = 1.5, 0.7 Hz, 1H), 6.69 (d, J = 2.0 Hz, 1H), 5.08 (d, J = 5.8 Hz, 1H), 4.64 (t, J = 6.1 Hz, 1H), 3.89 (s, 3H), 3.19 (dd, J = 18.3, 5.2 Hz, 1H), 2.74-2.58 (m, 1H), 2.28-2.12 (m, 2H), 1.85-1.74 (m, 1H), 1.68 (dd, J = 10.1, 6.1 Hz, 1H). |
| | (6S,9R)-N-(5-Chloro-2-fluoro-4- | | |
| | (2-methoxypyridin-4-yl)phenyl)- | | |
| | 3-oxo-3,5,6,7,8,9-hexahydro- | | |
| | 2H-6,9- | | |
| | epiminocyclohepta[c]pyridazine- | | |
| | 10-carboxamide | | |
| 202^{(d)(j)} | 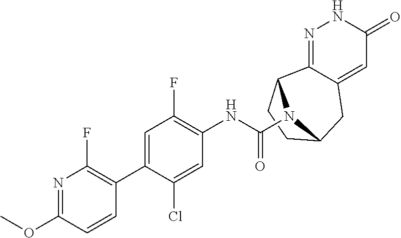 [SMILES: COc1ccc(-c2cc(F)c(NC(=O)N3[C@H]4CC[C@@H]3c3n[nH]c(=O)cc3C4)cc2Cl)c(F)n1] | Method 1 474.3, 476.3 at 1.35 min | ¹H NMR (400 MHz, DMSO-d6) δ 12.72 (s, 1H), 8.90 (s, 1H), 7.87 (dd, J = 10.0, 8.2 Hz, 1H), 7.78 (d, J = 7.4 Hz, 1H), 7.39 (d, J = 11.0 Hz, 1H), 6.88 (dd, J = 8.1, 1.2 Hz, 1H), 6.69 (s, 1H), 5.08 (d, J = 5.7 Hz, 1H), 4.65 (d, J = 6.1 Hz, 1H), 3.89 (s, 3H), 3.25-3.15 (m, 1H), 2.65 (d, J = 18.1 Hz, 1H), 2.28-2.12 (m, 2H), 1.80 (t, J = 9.6 Hz, 1H), 1.68 (t, J = 8.1 Hz, 1H). |
| | (6S,9R)-N-(5-Chloro-2-fluoro-4- | | |
| | (2-fluoro-6-methoxypyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 203^{(d)(j)} | 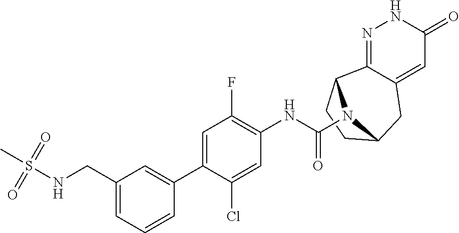 [SMILES: CS(=O)(=O)NCc1cccc(-c2cc(F)c(NC(=O)N3[C@H]4CC[C@@H]3c3n[nH]c(=O)cc3C4)cc2Cl)c1] | Method 1 532.2, 534.2 at 1.12 min | ¹H NMR (400 MHz, DMSO-d6) δ 12.63 (s, 1H), 8.85 (s, 1H), 7.76 (d, J = 7.3 Hz, 1H), 7.45 (t, J = 7.5 Hz, 1H), 7.42-7.33 (m, 3H), 7.29 (d, J = 11.0 Hz, 1H), 6.69 (s, 1H), 5.08 (d, J = 5.6 Hz, 1H), 4.64 (s, 1H), 4.21 (s, 2H), 3.26-3.14 (m, 1H), 2.86 (s, 3H), 2.73-2.60 (m, 1H), 2.19 (dd, J = 11.7, 6.4 Hz, 2H), 1.86- 1.73 (m, 1H), 1.68 (t, J = 8.3 Hz, 1H). one exchangeable proton not observed |
| | (6S,9R)-N-(2-Chloro-5-fluoro-3′- | | |
| | (methylsulfonamidomethyl)- | | |
| | [1,1′-biphenyl]-4-yl)-3-oxo- | | |
| | 3,5,6,7,8,9-hexahydro-2H-6,9- | | |
| | epiminocyclohepta[c]pyridazine- | | |
| | 10-carboxamide | | |
| 207^{(d)(j)} | 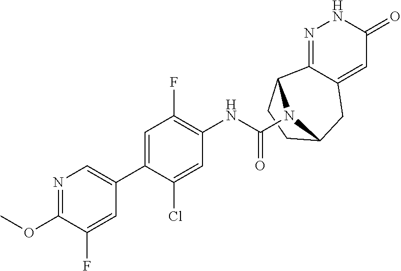 [SMILES: COc1ncc(-c2cc(F)c(NC(=O)N3[C@H]4CC[C@@H]3c3n[nH]c(=O)cc3C4)cc2Cl)cc1F] | Method 2 474.3, 476.3 at 1.67 min | ¹H NMR (400 MHz, DMSO-d6) δ 12.73 (s, 1H), 8.89 (s, 1H), 8.24 (d, J = 3.0 Hz, 1H), 7.73 (dd, J = 8.0, 2.8 Hz, 2H), 7.35 (d, J = 10.9 Hz, 1H), 6.69 (s, 1H), 5.07 (d, J = 5.6 Hz, 1H), 4.64 (s, 1H), 3.82 (s, 3H), 3.23-3.16 (m, 1H), 2.65 (d, J = 18.1 Hz, 1H), 2.25-2.15 (m, 2H), 1.80 (t, J = 9.7 Hz, 1H), 1.68 (t, J = 8.6 Hz, 1H). |
| | (6S,9R)-N-(5-Chloro-2-fluoro-4- | | |
| | (5-fluoro-6-methoxypyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 208^{(d)(j)} | 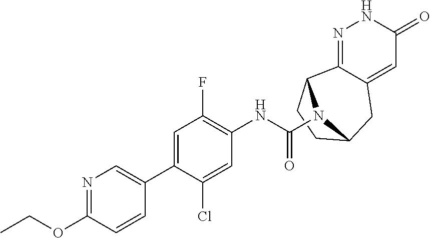 [SMILES: CCOc1ccc(-c2cc(F)c(NC(=O)N3[C@H]4CC[C@@H]3c3n[nH]c(=O)cc3C4)cc2Cl)cn1] | Method 1 470.3, 472.4 at 1.26 min | ¹H NMR (400 MHz, DMSO-d6) δ 12.73 (s, 1H), 8.88 (s, 1H), 8.20 (dd, J = 2.5, 0.8 Hz, 1H), 7.79 (dd, J = 8.6, 2.6 Hz, 1H), 7.75 (d, J = 7.3 Hz, 1H), 7.37 (d, J = 11.1 Hz, 1H), 6.87 (dd, J = 8.5, 0.8 Hz, 1H), 6.69 (s, 1H), 5.07 (d, J = 5.8 Hz, 1H), 4.64 (s, 1H), 4.34 (q, J = 7.0 Hz, 2H), 3.24-3.15 (m, 1H), 2.65 (d, J = 18.1 Hz, 1H), 2.19 (dd, J = 11.6, 6.5 Hz, 2H), 1.80 (t, J = 9.6 Hz, 1H), 1.68 (t, J = 9.2 Hz, 1H), 1.34 (t, J = 7.0 Hz, 3H). |
| | (6S,9R)-N-(5-Chloro-4-(6- | | |
| | ethoxypyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 209^{(d)(j)} | 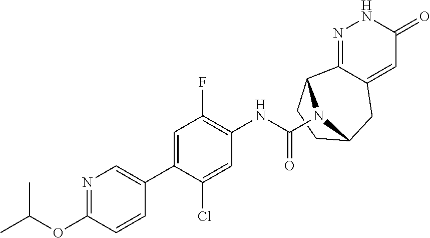 [SMILES: CC(C)Oc1ccc(-c2cc(F)c(NC(=O)N3[C@H]4CC[C@@H]3c3n[nH]c(=O)cc3C4)cc2Cl)cn1] | Method 1 484.3, 486.3 at 1.39 min | ¹H NMR (400 MHz, DMSO-d6) δ 12.73 (d, J = 1.9 Hz, 1H), 8.88 (s, 1H), 8.20 (dd, J = 2.6, 0.8 Hz, 1H), 7.79-7.73 (m, 2H), 7.37 (d, J = 11.1 Hz, 1H), 6.82 (dd, J = 8.6, 0.8 Hz, 1H), 6.69 (s, 1H), 5.28 (hept, J = 6.1 Hz, 1H), 5.07 (d, J = 5.6 Hz, 1H), 4.64 (d, J = 6.3 Hz, 1H), 3.19 (dd, J = 18.3, 5.3 Hz, 1H), 2.65 (d, J = 18.2 Hz, 1H), 2.27-2.13 (m, 2H), 1.84-1.75 (m, 1H), 1.68 (dd, J = 10.4, 6.4 Hz, 1H), 1.31 (d, J = 6.2 Hz, 6H). |
| | (6S,9R)-N-(5-Chloro-2-fluoro-4- | | |
| | (6-isopropoxypyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 210^{(d)(j)} | 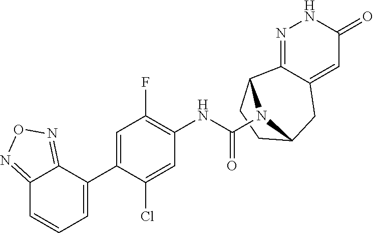 [SMILES: O=C(Nc1cc(Cl)c(-c2cccc3nonc23)cc1F)N1[C@H]2CC[C@@H]1c1n[nH]c(=O)cc1C2] | Method 1 467.0, 469.4 at 1.20 min | ¹H NMR (400 MHz, DMSO-d6) δ 8.12 (dd, J = 9.1, 0.8 Hz, 1H), 7.89 (d, J = 7.2 Hz, 1H), 7.73 (dd, J = 9.1, 6.7 Hz, 1H), 7.63 (dd, J = 6.7, 0.8 Hz, 1H), 7.56 (d, J = 11.1 Hz, 1H), 6.69 (s, 1H), 5.10 (d, J = 5.7 Hz, 1H), 4.67 (d, J = 6.3 Hz, 1H), 3.22 (dd, J = 18.2, 5.5 Hz, 1H), 2.66 (d, J = 18.1 Hz, 1H), 2.20 (dd, J = 11.5, 6.4 Hz, 2H), 1.81 (t, J = 9.5 Hz, 1H), 1.68 (t, J = 8.6 Hz, 1H). 2 exchangeable protons not observed |
| | (6S,9R)-N-(4- | | |
| | (Benzo[c][1,2,5]oxadiazol-4-yl)- | | |
| | 5-chloro-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 211^{(d)(j)} | 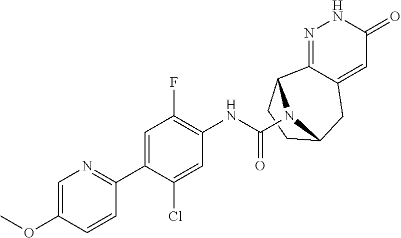 [SMILES: COc1ccc(-c2cc(F)c(NC(=O)N3[C@H]4CC[C@@H]3c3n[nH]c(=O)cc3C4)cc2Cl)nc1] | Method 1 456.3, 458.3 at 1.00 min | ¹H NMR (400 MHz, DMSO-d6) δ 12.75 (s, 1H), 8.90 (s, 1H), 8.41-8.36 (m, 1H), 7.75 (d, J = 7.2 Hz, 1H), 7.69-7.63 (m, 1H), 7.49 (dd, J = 8.8, 3.0 Hz, 1H), 7.42 (d, J = 11.3 Hz, 1H), 6.69 (s, 1H), 5.08 (d, J = 5.7 Hz, 1H), 4.69-4.60 (m, 1H), 3.88 (s, 3H), 3.20 (dd, J = 18.4, 5.3 Hz, 1H), 2.65 (d, J = 18.1 Hz, 1H), 2.27-2.12 (m, 2H), 1.85-1.75 (m, 1H), 1.68 (dd, J = 10.8, 6.5 Hz, 1H). |
| | (6S,9R)-N-(5-Chloro-2-fluoro-4- | | |
| | (5-methoxypyridin-2-yl)phenyl)- | | |
| | 3-oxo-3,5,6,7,8,9-hexahydro- | | |
| | 2H-6,9- | | |
| | epiminocyclohepta[c]pyridazine- | | |
| | 10-carboxamide | | |
| 212^{(d)(j)} | 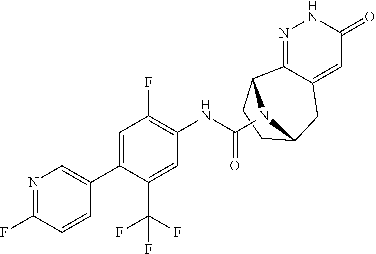 [SMILES: O=C(Nc1cc(C(F)(F)F)c(-c2ccc(F)nc2)cc1F)N1[C@H]2CC[C@@H]1c1n[nH]c(=O)cc1C2] | Method 1 478.1 at 1.15 min | ¹H NMR (400 MHz, DMSO-d6) δ 12.75 (d, J = 2.2 Hz, 1H), 9.05 (s, 1H), 8.21 (d, J = 2.5 Hz, 1H), 8.09 (d, J = 7.6 Hz, 1H), 7.99 (td, J = 8.2, 2.5 Hz, 1H), 7.45 (d, J = 10.9 Hz, 1H), 7.30 (dd, J = 8.4, 2.6 Hz, 1H), 6.70 (s, 1H), 5.10 (d, J = 5.7 Hz, 1H), 4.67 (d, J = 6.3 Hz, 1H), 3.25-3.18 (m, 1H), 2.67 (d, J = 18.2 Hz, 1H), 2.20 (dd, J = 11.7, 6.4 Hz, 2H), 1.81 (t, J = 9.7 Hz, 1H), 1.70 (d, J = 11.6 Hz, 1H). |
| | (6S,9R)-N-(2-Fluoro-4-(6- | | |
| | fluoropyridin-3-yl)-5- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 213^{(d)(j)} | 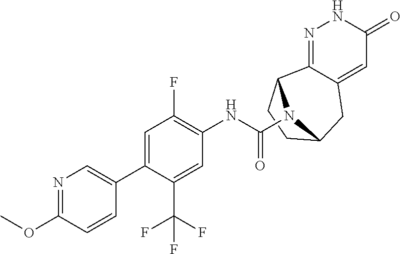 [SMILES: COc1ccc(-c2cc(F)c(NC(=O)N3[C@H]4CC[C@@H]3c3n[nH]c(=O)cc3C4)cc2C(F)(F)F)cn1] | Method 1 490.3 at 1.23 min | ¹H NMR (400 MHz, DMSO-d6) δ 12.74 (s, 1H), 9.01 (s, 1H), 8.11 (d, J = 2.4 Hz, 1H), 8.05 (d, J = 7.6 Hz, 1H), 7.68 (dd, J = 8.6, 2.5 Hz, 1H), 7.37 (d, J = 11.0 Hz, 1H), 6.93-6.87 (m, 1H), 6.70 (s, 1H), 5.09 (d, J = 5.6 Hz, 1H), 4.66 (s, 1H), 3.89 (s, 3H), 3.24 (s, 1H), 3.16 (d, J = 5.3 Hz, 1H), 2.66 (d, J = 18.3 Hz, 1H), 2.26-2.16 (m, 2H), 1.80 (d, J = 11.3 Hz, 1H), 1.70 (d, J = 11.8 Hz, 1H). |
| | (6S,9R)-N-(2-Fluoro-4-(6- | | |
| | methoxypyridin-3-yl)-5- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 215^{(d)(j)} | 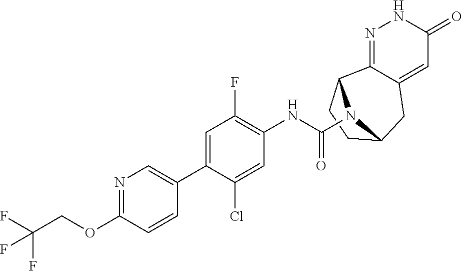 [SMILES: O=C(Nc1cc(Cl)c(-c2ccc(OCC(F)(F)F)nc2)cc1F)N1[C@H]2CC[C@@H]1c1n[nH]c(=O)cc1C2] | Method 1 524.4, 526.4 at 1.41 min | ¹H NMR (400 MHz, DMSO-d6) δ 12.72 (s, 1H), 8.89 (s, 1H), 8.27 (d, J = 2.3 Hz, 1H), 7.91 (dd, J = 8.6, 2.5 Hz, 1H), 7.78 (d, J = 7.3 Hz, 1H), 7.41 (d, J = 11.0 Hz, 1H), 7.08 (d, J = 8.6 Hz, 1H), 6.69 (s, 1H), 5.11-4.99 (m, 3H), 4.64 (t, J = 5.9 Hz, 1H), 3.20 (dd, J = 18.3, 5.3 Hz, 1H), 2.65 (d, J = 18.1 Hz, 1H), 2.29- 2.11 (m, 2H), 1.85-1.75 (m, 1H), 1.74-1.64 (m, 1H). |
| | (6S,9R)-N-(5-Chloro-2-fluoro-4- | | |
| | (6-(2,2,2-trifluoroethoxy)pyridin- | | |
| | 3-yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 216^{(d)(j)} | 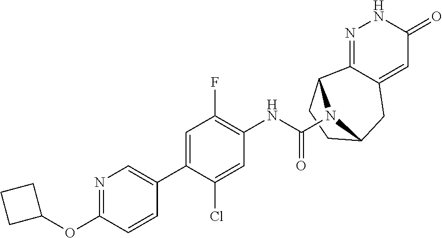 [SMILES: O=C(Nc1cc(Cl)c(-c2ccc(OC3CCC3)nc2)cc1F)N1[C@H]2CC[C@@H]1c1n[nH]c(=O)cc1C2] | Method 1 496.5, 498.5 at 1.45 min | ¹H NMR (400 MHz, DMSO-d6) δ 12.72 (s, 1H), 8.87 (s, 1H), 8.18 (dd, J = 2.5, 0.7 Hz, 1H), 7.79 (dd, J = 8.6, 2.5 Hz, 1H), 7.75 (d, J = 7.3 Hz, 1H), 7.37 (d, J = 11.1 Hz, 1H), 6.85 (dd, J = 8.6, 0.7 Hz, 1H), 6.69 (s, 1H), 5.17 (p, J = 7.4 Hz, 1H), 5.07 (d, J = 5.8 Hz, 1H), 4.67-4.60 (m, 1H), 3.19 (dd, J = 18.4, 5.3 Hz, 1H), 2.69-2.60 (m, 1H), 2.47- 2.37 (m, 2H), 2.28-2.15 (m, 2H), 2.15-2.00 (m, 2H), 1.85- 1.74 (m, 2H), 1.73-1.57 (m, 2H). |
| | (6S,9R)-N-(5-Chloro-4-(6- | | |
| | cyclobutoxypyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 218^{(d)(j)} | 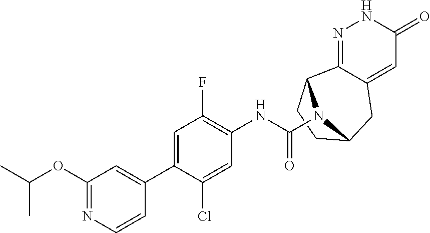 [SMILES: CC(C)Oc1cc(-c2cc(F)c(NC(=O)N3[C@H]4CC[C@@H]3c3n[nH]c(=O)cc3C4)cc2Cl)ccn1] | Method 1 484.2, 486.2 at 1.32 min | ¹H NMR (400 MHz, DMSO-d6) δ 12.72 (s, 1H), 8.92 (s, 1H), 8.21 (d, J = 5.3 Hz, 1H), 7.79 (d, J = 7.2 Hz, 1H), 7.39 (d, J = 11.0 Hz, 1H), 7.01 (dd, J = 5.3, 1.5 Hz, 1H), 6.77 (s, 1H), 6.69 (s, 1H), 5.28 (hept, J = 6.1 Hz, 1H), 5.08 (d, J = 5.5 Hz, 1H), 4.64 (t, J = 6.0 Hz, 1H), 3.19 (dd, J = 18.2, 5.3 Hz, 1H), 2.65 (d, J = 18.1 Hz, 1H), 2.25-2.13 (m, 2H), 1.87-1.75 (m, 1H), 1.72- 1.61 (m, 1H), 1.31 (d, J = 6.2 Hz, 6H). |
| | (6S,9R)-N-(5-Chloro-2-fluoro-4- | | |
| | (2-isopropoxypyridin-4- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 221^{(d)(j)} | 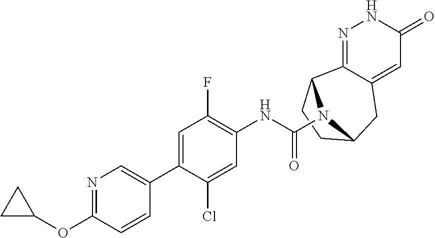 [SMILES: O=C(Nc1cc(Cl)c(-c2ccc(OC3CC3)nc2)cc1F)N1[C@H]2CC[C@@H]1c1n[nH]c(=O)cc1C2] | Method 1 482.3, 484.3 at 1.21 min | ¹H NMR (400 MHz, DMSO- d6) δ 12.74 (s, 1H), 8.89 (s, 1H), 8.25 (d, J = 2.5 Hz, 1H), 7.83 (dd, J = 8.6, 2.5 Hz, 1H), 7.76 (d, J = 7.3 Hz, 1H), 7.39 (d, J = 11.1 Hz, 1H), 6.95 (d, J = 8.5 Hz, 1H), 6.69 (s, 1H), 5.07 (d, J = 5.6 Hz, 1H), 4.64 (s, 1H), 4.24 (tt, J = 6.2, 3.1 Hz, 1H), 3.22- 3.16 (m, 1H), 2.65 (d, J = 18.0 Hz, 1H), 2.25-2.15 (m, 2H), 1.79 (d, J = 11.3 Hz, 1H), 1.69 (d, J = 9.5 Hz, 1H), 0.82-0.74 (m, 2H), |
| | (6S,9R)-N-(5-Chloro-4-(6- | | 0.70 (dq, J = 7.0, 3.3 Hz, |
| | cyclopropoxypyridin-3-yl)-2- | | 2H). |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 224^{(d)(o)} | 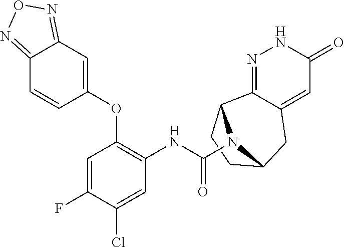 [SMILES: O=C(Nc1cc(Cl)c(F)cc1Oc1ccc2nonc2c1)N1[C@H]2CC[C@@H]1c1n[nH]c(=O)cc1C2] | Method 3 482.9 at 1.25 min | ¹H NMR (400 MHz, DMSO- d6) δ 12.55 (s, 1H), 8.69 (s, 1H), 8.04 (d, J = 9.6 Hz, 1H), 7.77 (d, J = 7.9 Hz, 1H), 7.51 (d, J = 9.7 Hz, 1H), 7.35 (dd, J = 9.6, 2.2 Hz, 1H), 6.96 (d, J = 2.2 Hz, 1H), 6.33 (s, 1H), 4.90 (d, J = 5.3 Hz, 1H), 4.44 (s, 1H), 2.91- 2.69 (m, 1H), 2.43 (d, J = 18.1 Hz, 1H), 2.01 (s, 2H), 1.67 (t, J = 9.5 Hz, 1H), 1.55 (d, J = 10.2 Hz, 1H). |
| | (6S,9R)-N-(2-(Benzo[c][1,2,5]- | | |
| | oxadiazol-5-yloxy)-5-chloro-4- | | |
| | fluorophenyl)-3-oxo-3,5,6,7,8,9- | | |
| | hexahydro-2H-6,9-epiminocyclo- | | |
| | hepta[c]pyridazine-10- | | |
| | carboxamide | | |
| 229^{(d)(j)} | 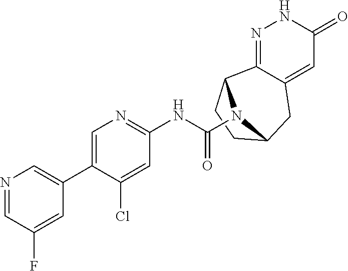 [SMILES: O=C(Nc1cc(Cl)c(-c2cncc(F)c2)cn1)N1[C@H]2CC[C@@H]1c1n[nH]c(=O)cc1C2] | Method 1 427.2, 429.1 at 0.98 min | ¹H NMR (400 MHz, DMSO- d6) δ 8.65 (d, J = 2.8 Hz, 1H), 8.57 (t, J = 1.8 Hz, 1H), 8.38 (s, 1H), 8.10 (s, 1H), 7.95 (ddd, J = 9.9, 2.8, 1.8 Hz, 1H), 6.70-6.65 (m, 1H), 5.24-5.19 (m, 1H), 4.78-4.73 (m, 1H), 3.19 (dd, J = 18.5, 5.0 Hz, 2H), 2.65 (d, J = 18.2 Hz, 1H), 2.18 (dd, J = 11.4, 6.2 Hz, 2H), 1.80 (t, J = 9.6 Hz, 1H), 1.68 (t, J = 8.2 Hz, 1H). 1H exchangeable proton not observed. |
| | (6S,9R)-N-(4-Chloro-5′-fluoro- | | |
| | [3,3′-bipyridin]-6-yl)-3-oxo- | | |
| | 3,5,6,7,8,9-hexahydro-2H-6,9- | | |
| | epiminocyclohepta[c]pyridazine- | | |
| | 10-carboxamide | | |
| 230^{(d)(j)} | 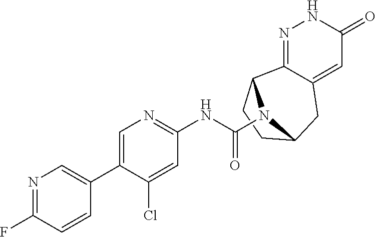 [SMILES: O=C(Nc1cc(Cl)c(-c2ccc(F)nc2)cn1)N1[C@H]2CC[C@@H]1c1n[nH]c(=O)cc1C2] | Method 1 427.1, 429.4 at 1.01 min | ¹H NMR (400 MHz, DMSO- d6) δ 12.73 (s, 1H), 9.99 (s, 1H), 8.37-8.31 (m, 2H), 8.14 (td, J = 8.2, 2.6 Hz, 1H), 8.10 (s, 1H), 7.32 (dd, J = 8.5, 2.8 Hz, 1H), 6.67 (s, 1H), 5.21 (d, J = 3.4 Hz, 1H), 4.89-4.60 (m, 1H), 3.19 (dd, J = 18.5, 5.3 Hz, 1H), 2.65 (d, J = 18.2 Hz, 1H), 2.23-2.10 (m, 2H), 1.80 (t, J = 9.7 Hz, 1H), 1.67 (t, J = 8.5 Hz, 1H). |
| | (6S,9R)-N-(4-Chloro-6′-fluoro- | | |
| | [3,3′-bipyridin]-6-yl)-3-oxo- | | |
| | 3,5,6,7,8,9-hexahydro-2H-6,9- | | |
| | epiminocyclohepta[c]pyridazine- | | |
| | 10-carboxamide | | |
| 234^{(d)(j)} | 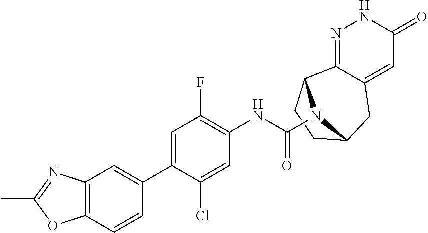 [SMILES: Cc1nc2cc(-c3cc(F)c(NC(=O)N4[C@H]5CC[C@@H]4c4n[nH]c(=O)cc4C5)cc3Cl)ccc2o1] | Method 1 480.1, 482.1 at 1.14 min | ¹H NMR (400 MHz, DMSO- d6) δ 12.72 (d, J = 2.2 Hz, 1H), 8.87 (s, 1H), 7.79- 7.66 (m, 3H), 7.42-7.32 (m, 2H), 6.69 (s, 1H), 5.08 (d, J = 5.8 Hz, 1H), 4.65 (d, J = 6.4 Hz, 1H), 3.21 (dd, J = 18.3, 5.2 Hz, 1H), 2.64 (s, 4H), 2.19 (dd, J = 11.5, 6.4 Hz, 2H), 1.80 (t, J = 9.7 Hz, 1H), 1.68 (t, J = 8.3 Hz, 1H). |
| | (6S,9R)-N-(5-Chloro-2-fluoro-4- | | |
| | (2-methylbenzo[d]oxazol-5- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |

| | | | |
|---|---|---|---|
| 235^{(d)(n)} | 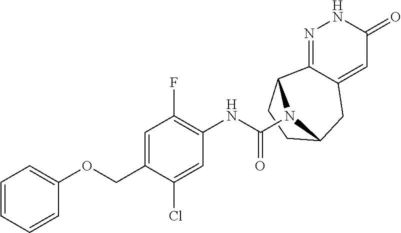 [SMILES: O=C(Nc1cc(Cl)c(COc2ccccc2)cc1F)N1[C@H]2CC[C@@H]1c1n[nH]c(=O)cc1C2] | Method 3 455.0 at 3.00 min | ¹H NMR (400 MHz, DMSO- d6) δ 12.70 (s, 1H), 8.81 (s, 1H), 7.70 (d, J = 7.2 Hz, 1H), 7.44 (d, J = 11.2 Hz, 1H), 7.31 (t, J = 7.2 Hz, 2H), 7.03- 6.94 (m, 3H), 6.67 (s, 1H), 5.07-5.05 (m, 3H), 4.64- 4.62 (m, 1H), 3.32-3.15 (m, 1H), 2.66-2.61 (m, 1H), 2.22-2.12 (m, 2H), 1.81- 1.76 (m, 1H), 1.69-1.65 (m, 1H). |
| | (6S,9R)-N-(5-Chloro-2-fluoro-4- | | |
| | (phenoxymethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 237^{(d)(n)} | 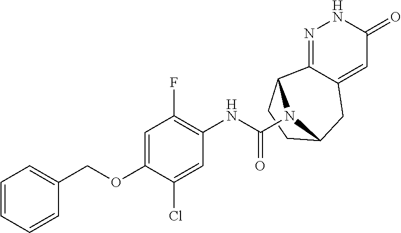 [SMILES: O=C(Nc1cc(Cl)c(OCc2ccccc2)cc1F)N1[C@H]2CC[C@@H]1c1n[nH]c(=O)cc1C2] | Method 3 455.1 at 2.85 min | ¹H NMR (400 MHz, DMSO- d6) δ 12.69 (s, 1H), 8.56 (s, 1H), 7.47-7.20 (m, 7H), 6.67 (s, 1H), 5.19 (s, 2H), 5.01-4.99 (d, J = 5.6 Hz, 1H), 4.59-4.55 (m, 1H), 3.20-3.14 (m, 1H), 2.64- 2.59 (d, J = 18.4 Hz, 1H), 2.24-2.10 (m, 2H), 1.79- 1.63 (m, 2H). |
| | (6S,9R)-N-(4-(Benzyloxy)-5- | | |
| | chloro-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 238^{(d)(j)} | 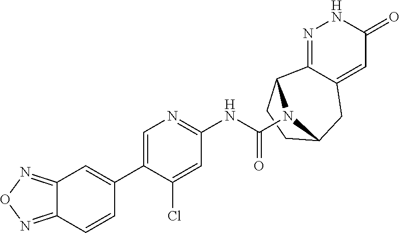 [SMILES: O=C(Nc1cc(Cl)c(-c2ccc3nonc3c2)cn1)N1[C@H]2CC[C@@H]1c1n[nH]c(=O)cc1C2] | Method 1 450.3, 452.2 at 1.18 min | ¹H NMR (400 MHz, DMSO- d6) δ 12.75 (s, 1H), 10.08 (s, 1H), 8.45 (s, 1H), 8.20 (t, J = 1.3 Hz, 1H), 8.15 (dd, J = 9.3, 1.0 Hz, 1H), 8.12 (s, 1H), 7.73 (dd, J = 9.3, 1.4 Hz, 1H), 6.68 (s, 1H), 5.22 (s, 1H), 4.76 (s, 1H), 3.20 (d, J = 18.6 Hz, 1H), 2.67 (dd, J = 3.6, 1.8 Hz, 1H), 2.19 (d, J = 6.1 Hz, 2H), 1.79 (d, J = 10.7 Hz, 1H), 1.69 (d, J = 11.0 Hz, 1H). |
| | (6S,9R)-N-(5-(Benzo[c][1,2,5]- | | |
| | oxadiazol-5-yl)-4-chloropyridin-2- | | |
| | yl)-3-oxo-3,5,6,7,8,9-hexahydro- | | |
| | 2H-6,9-epiminocyclo- | | |
| | hepta[c]pyridazine-10- | | |
| | carboxamide | | |
| 244^{(d)(j)} | 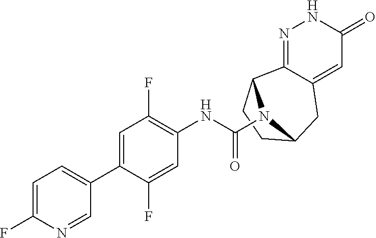 [SMILES: O=C(Nc1cc(F)c(-c2ccc(F)nc2)cc1F)N1[C@H]2CC[C@@H]1c1n[nH]c(=O)cc1C2] | Method 1 428.3 at 1.01 min | ¹H NMR (400 MHz, DMSO- d6) δ 12.72 (s, 1H), 8.91 (s, 1H), 8.43 (s, 1H), 8.18 (s, 1H), 7.60 (ddd, J = 25.0, 11.8, 7.0 Hz, 2H), 7.31 (dd, J = 8.6, 2.8 Hz, 1H), 6.69 (s, 1H), 5.09 (s, 1H), 4.66 (s, 1H), 3.19 (d, 1H), 2.63 (s, 1H), 2.20 (d, J = 6.6 Hz, 2H), 1.79 (d, J = 11.2 Hz, 1H), 1.68 (m, 1H). |
| | (6S,9R)-N-(2,5-Difluoro-4-(6- | | |
| | fluoropyridin-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 247^{(d)(j)} | 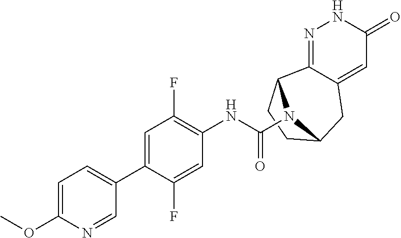 [SMILES: COc1ccc(-c2cc(F)c(NC(=O)N3[C@H]4CC[C@@H]3c3n[nH]c(=O)cc3C4)cc2F)cn1] | Method 1 440.3 at 1.09 min | ¹H NMR (400 MHz, DMSO- d6) δ 12.72 (s, 1H), 8.86 (s, 1H), 8.35 (s, 1H), 7.93- 7.86 (m, 1H), 7.57 (dd, J = 12.2, 6.7 Hz, 1H), 7.48 (dd, J = 11.3, 7.3 Hz, 1H), 6.92 (dd, J = 8.7, 0.8 Hz, (d, J = 5.7 Hz, 1H), 4.66 (d, J = 6.1 Hz, 1H), 3.89 (s, 3H), 3.23-3.14 (m, 1H), 2.65 (d, J = 18.1 Hz, 1H), 2.18 (dd, J = 11.7, 6.6 Hz, 2H), 1.80 (t, J = 9.3 Hz, 1H), 1.68 (t, J = 8.6 Hz, 1H). |
| | (6S,9R)-N-(2,5-Difluoro-4-(6- | | |
| | methoxypyridin-3-yl)phenyl)-3- | | |
| | oxo-3,5,6,7,8,9-hexahydro-2H- | | |
| | 6,9- | | |
| | epiminocyclohepta[c]pyridazine- | | |
| | 10-carboxamide | | |
| 254^{(d)(j)} | 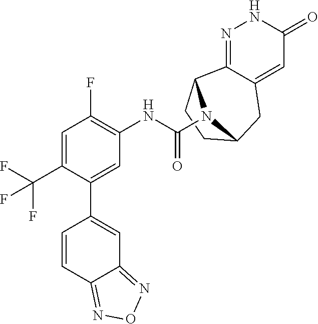 [SMILES: O=C(Nc1cc(-c2ccc3nonc3c2)c(C(F)(F)F)cc1F)N1[C@H]2CC[C@@H]1c1n[nH]c(=O)cc1C2] | Method 1 501.2 at 1.33 min | ¹H NMR (400 MHz, DMSO- d6) δ 12.73 (s, 1H), 9.16 (s, 1H), 8.13 (dd, J = 9.2, 1.1 Hz, 1H), 8.03 (d, J = 1.3 Hz, 1H), 7.84 (s, 1H), 7.81 (d, J = 4.0 Hz, 1H), 7.53 (d, J = 9.3 Hz, 1H), 6.67 (s, 1H), 5.08 (d, J = 5.7 Hz, 1H), 4.64 (s, 1H), 3.18 (dd, J = 11.6, 5.4 Hz, 1H), 2.65 (d, J = 18.0 Hz, 1H), 2.17 (dd, J = 11.5, 6.3 Hz, 2H), 1.79 (t, J = 9.5 Hz, 1H), 1.67 (t, J = 8.6 Hz, 1H). |
| | (6S,9R)-N-(5-(Benzo[c][1,2,5]- | | |
| | oxadiazol-5-yl)-2-fluoro-4- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 257^{(d)(j)} | 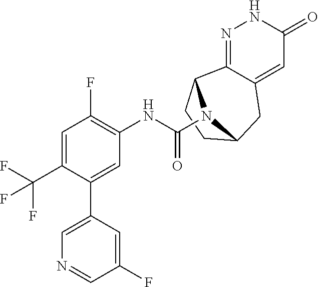 [SMILES: O=C(Nc1cc(-c2cncc(F)c2)c(C(F)(F)F)cc1F)N1[C@H]2CC[C@@H]1c1n[nH]c(=O)cc1C2] | Method 1 478.3 at 1.10 min | ¹H NMR (400 MHz, DMSO- d6) δ 12.74 (s, 1H), 9.14 (s, 1H), 8.67 (d, J = 2.8 Hz, 1H), 8.38 (s, 1H), 7.89-7.70 (m, 3H), 6.67 (s, 1H), 5.08 (d, J = 5.5 Hz, 1H), 4.65 (d, J = 6.2 Hz, 1H), 3.18 (dd, J = 11.7, 5.3 Hz, 1H), 2.65 (d, J = 18.1 Hz, 1H), 2.17 (dd, J = 11.7, 6.4 Hz, 2H), 1.83- 1.74 (m, 1H), 1.67 (t, J = 9.4 Hz, 1H). |
| | (6S,9R)-N-(2-Fluoro-5-(5- | | |
| | fluoropyridin-3-yl)-4- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 258^{(d)(j)} | 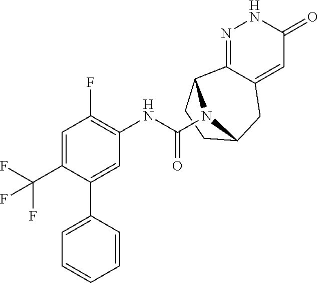 [SMILES: O=C(Nc1cc(-c2ccccc2)c(C(F)(F)F)cc1F)N1[C@H]2CC[C@@H]1c1n[nH]c(=O)cc1C2] | Method 1 459.2 at 1.34 min | ¹H NMR (400 MHz, DMSO- d6) δ 12.73 (d, J = 2.3 Hz, 1H), 9.05 (s, 1H), 7.71 (d, J = 11.3 Hz, 1H), 7.66 (d, J = 7.6 Hz, 1H), 7.42 (dd, J = 5.0, 1.9 Hz, 3H), 7.27 (dd, J = 6.7, 2.9 Hz, 2H), 6.67 (s, 1H), 5.07 (d, J = 5.6 Hz, 1H), 4.63 (s, 1H), 3.21-3.14 (m, 1H), 2.64 (d, J = 18.2 Hz, 1H), 2.17 (dd, J = 11.6, 6.5 Hz, 2H), 1.82-1.74 (m, 1H), 1.71-1.62 (m, 1H). |
| | (6S,9R)-N-(4-Fluoro-6- | | |
| | (trifluoromethyl)-[1,1′-biphenyl]-3- | | |
| | yl)-3-oxo-3,5,6,7,8,9-hexahydro- | | |
| | 2H-6,9-epiminocyclohepta- | | |
| | [c]pyridazine-10-carboxamide | | |
| 266^{(d)(j)} | 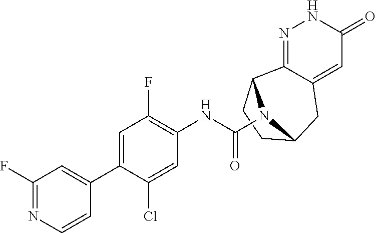 [SMILES: O=C(Nc1cc(Cl)c(-c2ccnc(F)c2)cc1F)N1[C@H]2CC[C@@H]1c1n[nH]c(=O)cc1C2] | Method 2 444.3, 446.2 at 1.53 min | ¹H NMR (400 MHz, DMSO- d6) δ 12.75 (d, J = 2.2 Hz, 1H), 8.98 (s, 1H), 8.33 (d, J = 5.2 Hz, 1H), 7.85 (d, J = 7.2 Hz, 1H), 7.53-7.44 (m, 2H), 7.35-7.30 (m, 1H), 6.69 (s, 1H), 5.08 (d, J = 5.6 Hz, 1H), 4.65 (s, 1H), 3.24- 3.15 (m, 1H), 2.66 (d, J = 18.1 Hz, 1H), 2.19 (dd, J = 11.8, 6.3 Hz, 2H), 1.80 (t, J = 9.6 Hz, 1H), 1.68 (s, 1H). |
| | (6S,9R)-N-(5-Chloro-2-fluoro-4- | | |
| | (2-fluoropyridin-4-yl)phenyl)-3- | | |
| | oxo-3,5,6,7,8,9-hexahydro-2H- | | |
| | 6,9-epiminocyclohepta- | | |
| | [c]pyridazine-10-carboxamide | | |
| 269^{(d)(j)} | 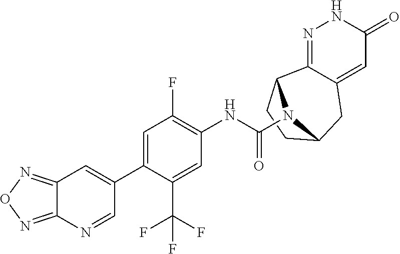 [SMILES: O=C(Nc1cc(C(F)(F)F)c(-c2cnc3nonc3c2)cc1F)N1[C@H]2CC[C@@H]1c1n[nH]c(=O)cc1C2] | Method 1 500.1, 501.3 at 1.69 min | ¹H NMR (400 MHz, DMSO- d6) δ 12.76 (s, 1H), 9.15 (s, 1H), 9.10-9.05 (m, 1H), 8.69 (d, J = 2.1 Hz, 1H), 8.19 (d, J = 7.4 Hz, 1H), 7.64 (d, J = 10.9 Hz, 1H), 6.73-6.68 (m, 1H), 5.12 (d, J = 5.6 Hz, 1H), 4.68 (s, 1H), 3.22 (dd, J = 18.1, 5.2 Hz, 1H), 3.16 (d, J = 5.1 Hz, 1H), 2.68 (d, J = 17.9 Hz, 1H), 2.21 (dd, J = 11.7, 6.5 Hz, 2H), 2.07 (s, 2H), 1.82 (t, J = 9.6 Hz, 1H), 1.71 (d, J = 11.8 Hz, 1H). |
| | (6S,9R)-N-(4- | | |
| | ([1,2,5]Oxadiazolo[3,4-b]pyridin- | | |
| | 6-yl)-2-fluoro-5- | | |
| | (trifluoromethyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 270^{(d)(j)} | 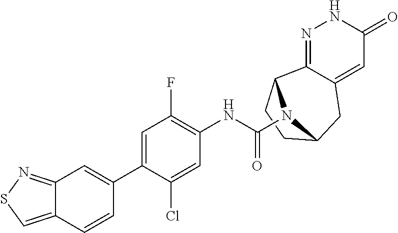 [SMILES: O=C(Nc1cc(Cl)c(-c2ccc3csnc3c2)cc1F)N1[C@H]2CC[C@@H]1c1n[nH]c(=O)cc1C2] | Method 1 482.3, 484.5 at 1.15 min | ¹H NMR (400 MHz, DMSO- d6) δ 12.75 (d, J = 2.3 Hz, 1H), 9.83 (d, J = 1.1 Hz, 1H), 8.94 (s, 1H), 7.98 (d, J = 8.8 Hz, 1H), 7.82-7.78 (m, 2H), 7.47 (d, J = 11.1 Hz, 1H), 7.35 (dd, J = 8.9, 1.5 Hz, 1H), 6.70 (s, 1H), 5.09 (d, J = 5.6 Hz, 1H), 4.66 (d, J = 6.3 Hz, 1H), 3.21 (dd, J = 18.7, 5.3 Hz, 1H), 2.66 (d, J = 18.2 Hz, 1H), 2.20 (dd, J = 11.6, 6.5 Hz, 2H), 1.81 (t, J = 9.6 Hz, 1H), 1.70 (d, J = 11.8 Hz, 1H). |
| | (6S,9R)-N-(4-(Benzo[c]isothiazol- | | |
| | 6-yl)-5-chloro-2-fluorophenyl)-3- | | |
| | oxo-3,5,6,7,8,9-hexahydro-2H- | | |
| | 6,9-epiminocyclohepta- | | |
| | [c]pyridazine-10-carboxamide | | |
| 271^{(d)(j)} | 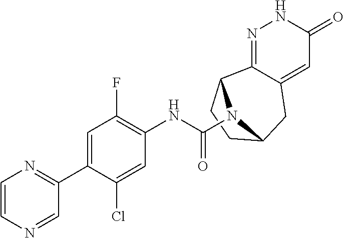 [SMILES: O=C(Nc1cc(Cl)c(-c2cnccn2)cc1F)N1[C@H]2CC[C@@H]1c1n[nH]c(=O)cc1C2] | Method 1 427.1, 429.1 at 0.90 min | ¹H NMR (400 MHz, DMSO- d6) δ 12.73 (br. s, 1H), 8.97 (br. s, 1H), 8.96 (d, J = 1.6 Hz, 1H), 8.77 (dd, J = 2.5, 1.6 Hz, 1H), 8.68 (d, J = 2.5 Hz, 1H), 7.87 (d, J = 7.1 Hz, 1H), 7.57 (d, J = 11.1 Hz, 1H), 6.69 (s, 1H), 5.10 (d, J = 5.8 Hz, 1H), 4.66 (t, J = 6.0 Hz, 1H), 3.20 (dd, J = 17.9, 5.0 Hz, 1H), 2.66 (d, J = 18.2 Hz, 1H), 2.29-2.12 (m, 2H), 1.85-1.76 (m, 1H), 1.73-1.64 (m, 1H). |
| | (6S,9R)-N-(5-Chloro-2-fluoro-4- | | |
| | (pyrazin-2-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 275^{(d)(j)} | 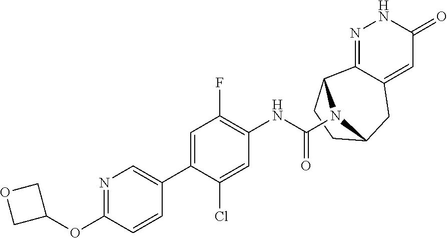 [SMILES: O=C(Nc1cc(Cl)c(-c2ccc(OC3COC3)nc2)cc1F)N1[C@H]2CC[C@@H]1c1n[nH]c(=O)cc1C2] | Method 1 498.2, 500.2 at 1.10 min | ¹H NMR (400 MHz, DMSO- d6) δ 12.72 (s, 1H), 8.88 (s, 1H), 8.17 (dd, J = 2.5, 0.8 Hz, 1H), 7.86 (dd, J = 8.6, 2.5 Hz, 1H), 7.76 (d, J = 7.3 Hz, 1H), 7.38 (d, J = 11.1 Hz, 1H), 7.00 (dd, J = 8.6, 0.7 Hz, 1H), 6.69 (s, 1H), 5.61 (tt, J = 6.1, 5.1 Hz, 1H), 5.07 (d, J = 5.8 Hz, 1H), 4.91 (t, J = 7.2 Hz, 2H), 4.64 (t, J = 6.1 Hz, 1H), 4.59 (dd, J = 8.0, 5.3 Hz, 2H), 3.19 (dd, J = 18.2, 5.3 Hz, 1H), 2.65 (d, J = 17.0 Hz, 1H), |
| | (6S,9R)-N-(5-Chloro-2-fluoro-4- | | 2.25-2.11 (m, 2H), 1.84- |
| | (6-(oxetan-3-yloxy)pyridin-3- | | 1.76 (m, 1H), 1.72-1.64 |
| | yl)phenyl)-3-oxo-3,5,6,7,8,9- | | (m, 1H). |
| | hexahydro-2H-6,9- | | |
| | epiminocyclohepta[c]pyridazine- | | |
| | 10-carboxamide | | |
| 277^{(d)(j)} | 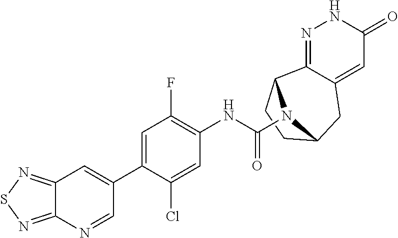 [SMILES: O=C(Nc1cc(Cl)c(-c2cnc3nsnc3c2)cc1F)N1[C@H]2CC[C@@H]1c1n[nH]c(=O)cc1C2] | Method 1 484.2, 486.2 at 1.02 min | ¹H NMR (400 MHz, DMSO- d6) δ 12.75 (s, 1H), 9.21 (d, J = 2.2 Hz, 1H), 9.01 (s, 1H), 8.68 (d, J = 2.3 Hz, 1H), 7.90 (d, J = 7.3 Hz, 1H), 7.65 (d, J = 11.0 Hz, 1H), 6.70 (s, 1H), 5.10 (d, J = 5.6 Hz, 1H), 4.70-4.64 (m, 1H), 3.25-3.18 (m, 1H), 2.67 (d, J = 18.4 Hz, 1H), 2.27-2.15 (m, 2H), 1.85- 1.77 (m, 1H), 1.74-1.66 (m, 1H). |
| | (6S,9R)-N-(4- | | |
| | ([1,2,5]Thiadiazolo[3,4-b]pyridin- | | |
| | 6-yl)-5-chloro-2-fluorophenyl)-3- | | |
| | oxo-3,5,6,7,8,9-hexahydro-2H- | | |
| | 6,9-epiminocyclohepta- | | |
| | [c]pyridazine-10-carboxamide | | |
| 278^{(d)(j)} | 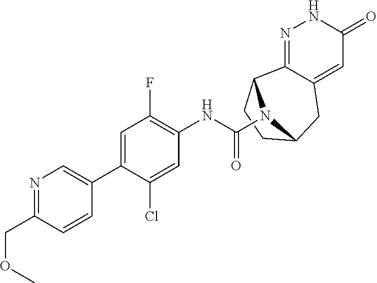 [SMILES: COCc1ccc(-c2cc(F)c(NC(=O)N3[C@H]4CC[C@@H]3c3n[nH]c(=O)cc3C4)cc2Cl)cn1] | Method 2 470.2, 472.2 at 0.99 min | ¹H NMR (400 MHz, DMSO- d6) δ 10.42 (s, 2H), 8.57 (dd, J = 2.3, 0.8 Hz, 1H), 7.90 (dd, J = 8.1, 2.3 Hz, 1H), 7.82 (d, J = 7.3 Hz, 1H), 7.49 (dd, J = 8.1, 0.8 Hz, 1H), 7.41 (d, J = 11.1 Hz, 1H), 6.68 (s, 1H), 5.08 (d, J = 5.8 Hz, 1H), 4.65 (t, J = 6.0 Hz, 1H), 4.55 (s, 2H), 3.40 (s, 3H), 3.26-3.10 (m, 1H), 2.70-2.60 (m, 1H), 2.28- 2.11 (m, 2H), 1.84-1.73 (m, 1H), 1.72-1.59 (m, 1H). |
| | (6S,9R)-N-(5-Chloro-2-fluoro-4- | | |
| | (6-(methoxymethyl)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 279^{(d)(j)} | 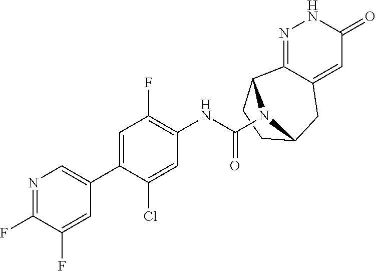 [SMILES: O=C(Nc1cc(Cl)c(-c2cnc(F)c(F)c2)cc1F)N1[C@H]2CC[C@@H]1c1n[nH]c(=O)cc1C2] | Method 2 462.1, 464.1 at 1.29 min | ¹H NMR (400 MHz, DMSO- d6) δ 12.73 (s, 1H), 8.95 (s, 1H), 8.31-8.20 (m, 1H), 8.16 (t, J = 1.9 Hz, 1H), 7.83 (d, J = 7.3 Hz, 1H), 7.49 (d, J = 11.1 Hz, 1H), 6.69 (s, 1H), 5.09 (d, J = 5.7 Hz, 1H), 4.66 (d, J = 6.3 Hz, 1H), 3.25-3.15 (m, 1H), 2.66 (d, J = 18.1 Hz, 1H), 2.29-2.11 (m, 2H), 1.85-1.76 (m, 1H), 1.73-1.61 (m, 1H). |
| | (6S,9R)-N-(5-Chloro-4-(5,6- | | |
| | difluoropyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 280^{(d)(j)} | 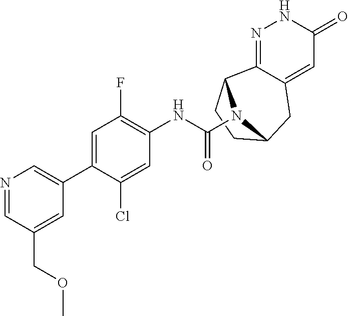 [SMILES: COCc1cncc(-c2cc(F)c(NC(=O)N3[C@H]4CC[C@@H]3c3n[nH]c(=O)cc3C4)cc2Cl)c1] | Method 2 470.2, 472.1 at 0.94 min | ¹H NMR (400 MHz, DMSO- d6) δ 12.72 (s, 1H), 8.92 (s, 1H), 8.67-8.45 (m, 2H), 7.90-7.69 (m, 2H), 7.44 (d, J = 11.1 Hz, 1H), 6.69 (d, J = 1.8 Hz, 1H), 5.09 (d, J = 5.7 Hz, 1H), 4.65 (t, J = 6.1 Hz, 1H), 4.52 (s, 2H), 3.34 (s, 3H), 3.20 (dd, J = 18.3, 5.3 Hz, 1H), 2.66 (d, J = 18.1 Hz, 1H), 2.29-2.09 (m, 2H), 1.88-1.53 (m, 2H). |
| | (6S,9R)-N-(5-Chloro-2-fluoro-4- | | |
| | (5-(methoxymethyl)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 281^{(d)(j)} | 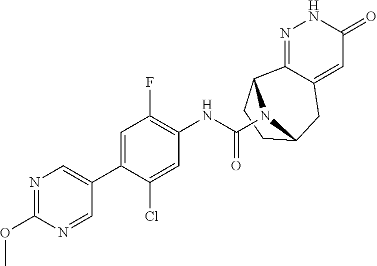 [SMILES: COc1ncc(-c2cc(F)c(NC(=O)N3[C@H]4CC[C@@H]3c3n[nH]c(=O)cc3C4)cc2Cl)cn1] | Method 1 457.2, 459.2 at 0.93 min | ¹H NMR (400 MHz, DMSO- d6) δ 12.72 (d, J = 2.3 Hz, 1H), 8.92 (s, 1H), 8.72 (s, 2H), 7.81 (d, J = 7.4 Hz, 1H), 7.49 (d, J = 11.1 Hz, 1H), 6.69 (d, J = 2.1 Hz, 1H), 5.08 (d, J = 5.7 Hz, 1H), 4.67-4.62 (m, 1H), 3.97 (s, 3H), 3.20 (dd, J = 19.0, 4.3 Hz, 1H), 2.65 (d, J = 18.3 Hz, 1H), 2.23-2.14 (m, 2H), 1.85-1.76 (m, 1H), 1.73-1.65 (m, 1H). |
| | (6S,9R)-N-(5-Chloro-2-fluoro-4- | | |
| | (2-methoxypyrimidin-5-yl)phenyl)- | | |
| | 3-oxo-3,5,6,7,8,9-hexahydro-2H- | | |
| | 6,9- | | |
| | epiminocyclohepta[c]pyridazine- | | |
| | 10-carboxamide | | |
| 282^{(d)(g)} | 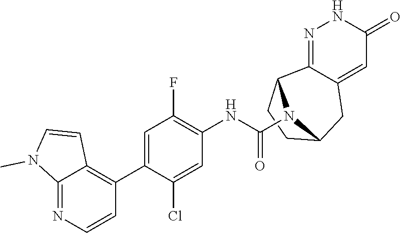 [SMILES: Cn1ccc2c(-c3cc(F)c(NC(=O)N4[C@H]5CC[C@@H]4c4n[nH]c(=O)cc4C5)cc3Cl)ccnc21] | Method 1 479.3, 481.3 at 1.03 min | ¹H NMR (400 MHz, DMSO- d6) δ 12.73 (s, 1H), 8.94 (s, 1H), 8.33 (d, J = 4.9 Hz, 1H), 7.85 (d, J = 7.2 Hz, 1H), 7.57 (d, J = 3.5 Hz, 1H), 7.37 (d, J = 10.9 Hz, 1H), 7.08 (d, J = 4.9 Hz, 1H), 6.70 (s, 1H), 6.23 (d, J = 3.5 Hz, 1H), 5.10 (d, J = 5.7 Hz, 1H), 4.67 (d, J = 6.2 Hz, 1H), 3.86 (s, 3H), 3.22 (dd, J = 18.3, 5.3 Hz, 1H), 2.66 (d, J = 18.2 Hz, 1H), 2.28-2.14 (m, 2H), 1.85-1.76 (m, 1H), 1.69 (dd, J = 10.5, 6.4 Hz, |
| | (6S,9R)-N-(5-Chloro-2-fluoro-4- | | 1H). |
| | (1-methyl-1H-pyrrolo[2,3- | | |
| | b]pyridin-4-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 286^{(d)(j)} | 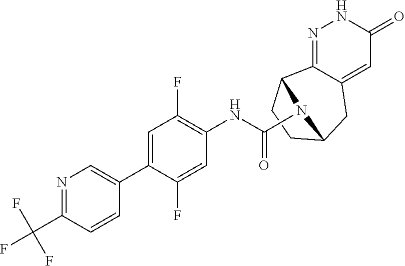 [SMILES: O=C(Nc1cc(F)c(-c2ccc(C(F)(F)F)nc2)cc1F)N1[C@H]2CC[C@@H]1c1n[nH]c(=O)cc1C2] | Method 1 478.0 at 1.26 min | ¹H NMR (400 MHz, DMSO- d6) δ 12.73 (d, J = 2.1 Hz, 1H), 9.00-8.94 (m, 2H), 8.28 (d, J = 8.2 Hz, 1H), 8.01 (dd, J = 8.3, 0.8 Hz, 1H), 7.68 (dt, J = 11.2, 7.1 Hz, 2H), 6.69 (s, 1H), 5.11 (d, J = 5.6 Hz, 1H), 4.67 (d, J = 6.2 Hz, 1H), 3.20 (dd, J = 18.3, 5.3 Hz, 1H), 2.66 (d, J = 18.2 Hz, 1H), 2.26-2.12 (m, 2H), 1.85-1.76 (m, 1H), 1.73-1.61 (m, 1H) |
| | (6S,9R)-N-(2,5-Difluoro-4-(6- | | |
| | (trifluoromethyl)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 291^{(d)(j)} | 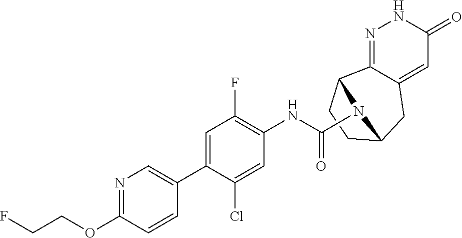 [SMILES: O=C(Nc1cc(Cl)c(-c2ccc(OCCF)nc2)cc1F)N1[C@H]2CC[C@@H]1c1n[nH]c(=O)cc1C2] | Method 1 487.7, 489.9 at 1.17 min | ¹H NMR (400 MHz, DMSO- d6) δ 12.74 (s, 1H), 8.89 (s, 1H), 8.22 (d, J = 2.7 Hz, 1H), 7.84 (dd, J = 8.6, 2.6 Hz, 1H), 7.76 (d, J = 7.3 Hz, 1H), 7.39 (d, J = 11.0 Hz, 1H), 6.96 (dd, J = 8.6, 0.8 Hz, 1H), 6.69 (s, 1H), 5.08 (d, J = 5.7 Hz, 1H), 4.88-4.78 (m, 1H), 4.75-4.68 (m, 1H), 4.64 (s, 1H), 4.62-4.55 (m, 1H), 4.55-4.47 (m, 1H), 3.20 (dd, J = 18.3, 3.9 Hz, 1H), 2.71-2.59 (m, 1H), 2.19 (dd, J = 11.7, 6.2 Hz, |
| | (6S,9R)-N-(5-Chloro-2-fluoro-4- | | 2H), 1.80 (t, J = 9.4 Hz, 1H), |
| | (6-(2-fluoroethoxy)pyridin-3- | | 1.75-1.62 (m, 1H) |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 292^{(d)(j)} | 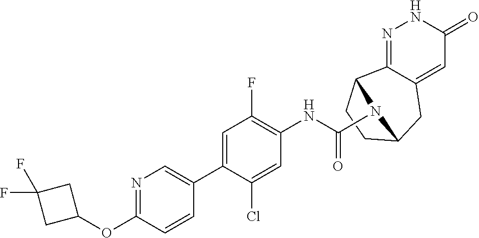 [SMILES: O=C(Nc1cc(Cl)c(-c2ccc(OC3CC(F)(F)C3)nc2)cc1F)N1[C@H]2CC[C@@H]1c1n[nH]c(=O)cc1C2] | Method 1 532.3, 534.3 at 1.43 min | ¹H NMR (400 MHz, DMSO- d6) δ 12.73 (s, 1H), 8.90 (s, 1H), 8.22 (dd, J = 2.6, 0.9 Hz, 1H), 7.85 (dd, J = 8.6, 2.5 Hz, 1H), 7.76 (d, J = 7.3 Hz, 1H), 7.38 (d, J = 11.1 Hz, 1H), 6.96 (dd, J = 8.6, 0.7 Hz, 1H), 6.69 (s, 1H), 5.20-5.12 (m, 1H), 5.07 (d, J = 5.7 Hz, 1H), 4.67-4.60 (m, 1H), 3.24-3.10 (m, 3H), 2.75 (qd, J = 14.0, 5.3 Hz, 2H), 2.65 (d, J = 18.2 Hz, 1H), 2.25-2.13 (m, 2H), 1.84-1.75 (m, 1H), 1.74- |
| | (6S,9R)-N-(5-Chloro-4-(6-(3,3- | | 1.64 (m, 1H). |
| | difluorocyclobutoxy)pyridin-3-yl)- | | |
| | 2-fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 293^{(d)(n)} | 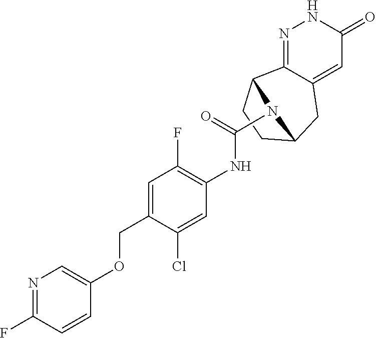 [SMILES: O=C(Nc1cc(Cl)c(COc2ccc(F)nc2)cc1F)N1[C@H]2CC[C@@H]1c1n[nH]c(=O)cc1C2] | Method 3 473.9 at 2.60 min | ¹H NMR (400 MHz, DMSO- d6) δ 12.70 (s, 1H), 8.83 (s, 1H), 8.00 (t, J = 2.4 Hz, 1H), 7.73-7.68 (m, 2H), 7.51 (d, J = 11.2 Hz, 1H), 7.16-7.12 (m, 1H), 6.67 (s, 1H), 5.14 (s, 2H), 5.06 (d, J = 5.6 Hz, 1H), 4.63 (d, J = 5.6 Hz, 1H), 3.18 (dd, J = 19.2, 5.2 Hz, 1H), 2.64 (d, J = 18.4 Hz, 1H), 2.25-1.12 (m, 2H), 1.81-1.76 (m, 1H), 1.69- 1.65 (m, 1H). |
| | (6S,9R)-N-(5-Chloro-2-fluoro-4- | | |
| | (((6-fluoropyridin-3- | | |
| | yl)oxy)methyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 294^{(d)(n)} | 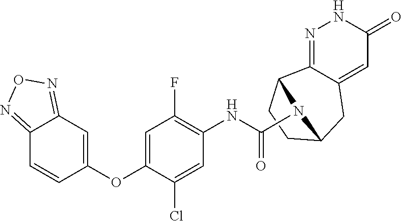 [SMILES: O=C(Nc1cc(Cl)c(Oc2ccc3nonc3c2)cc1F)N1[C@H]2CC[C@@H]1c1n[nH]c(=O)cc1C2] | Method 3 482.9 at 2.75 min | ¹H NMR (400 MHz, DMSO- d6) δ 12.73 (s, 1H), 8.88 (s, 1H), 8.15 (d, J = 9.6 Hz, 1H), 7.86 (d, J = 8.0 Hz, 1H), 7.55 (dd, J = 2.0 Hz, J = 9.6 Hz, 1H), 7.50 (d, J = 10.8 Hz, 1H), 7.03 (d, J = 1.6 Hz, 1H), 6.69 (s, 1H), 5.08 (d, J = 5.6 Hz, 1H), 4.63 (t, J = 6.0 Hz, 1H), 3.23-3.17 (m, 1H), 2.65 (d, J = 18.0 Hz, 1H), 2.24-2.16 (m, 2H), 1.82-1.78 (m, 1H), 1.72- 1.66 (m, 1H). |
| | (6S,9R)-N-(4- | | |
| | (Benzo[c][1,2,5]oxadiazol-5- | | |
| | yloxy)-5-chloro-2-fluorophenyl)-3- | | |
| | oxo-3,5,6,7,8,9-hexahydro-2H- | | |
| | 6,9-epiminocyclohepta- | | |
| | [c]pyridazine-10-carboxamide | | |
| 299^{(d)(j)} | 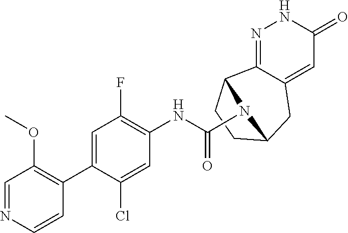 [SMILES: COc1cnccc1-c1cc(F)c(NC(=O)N2[C@H]3CC[C@@H]2c2n[nH]c(=O)cc2C3)cc1Cl] | Method 1 456.1, 458.2 at 0.94 min | ¹H NMR (500 MHz, DMSO- d6) δ 12.73 (s, 1H), 8.88 (s, 1H), 8.48 (s, 1H), 8.29 (d, J = 4.7 Hz, 1H), 7.73 (d, J = 7.2 Hz, 1H), 7.28 (d, J = 10.8 Hz, 1H), 7.23 (d, J = 4.7 Hz, 1H), 6.69 (s, 1H), 5.07 (d, J = 5.8 Hz, 1H), 4.64 (t, J = 6.2 Hz, 1H), 3.85 (s, 3H), 3.20 (dd, J = 18.2, 5.4 Hz, 1H), 2.65 (d, J = 18.3 Hz, 1H), 2.31-2.13 (m, 2H), 1.84-1.77 (m, 1H), 1.73-1.62 (m, 1H). |
| | (6S,9R)-N-(5-Chloro-2-fluoro-4- | | |
| | (3-methoxypyridin-4-yl)phenyl)-3- | | |
| | oxo-3,5,6,7,8,9-hexahydro-2H- | | |
| | 6,9-epiminocyclohepta- | | |
| | [c]pyridazine-10-carboxamide | | |
| 302^{(d)(j)} | 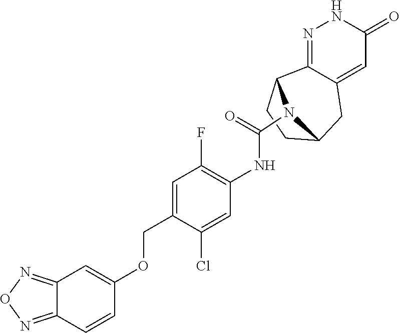 [SMILES: O=C(Nc1cc(Cl)c(COc2ccc3nonc3c2)cc1F)N1[C@H]2CC[C@@H]1c1n[nH]c(=O)cc1C2] | Method 3 496.8 at 2.86 min | ¹H NMR (400 MHz, DMSO- d6) δ 12.72 (s, 1H), 8.71 (s, 1H), 7.99 (d, J = 9.6 Hz 1H), 7.76 (d, J = 7.2 Hz, 1H), 7.60 (d, J = 10.8 Hz, 1H), 7.42 (d, J = 2 Hz, 1H), 7.38- 7.35 (m, 1H), 6.68 (s, 1H), 5.23 (s, 2H), 5.07 (d, J = 5.6 Hz, 1H), 4.64 (t, J = 6.0 Hz, 1H), 3.22-3.15 (m, 1H), 2.65 (d, J = 5.6 Hz, 1H), 2.26-2.13 (m, 2H), 1.85- 1.66 (m, 2H). |
| | (6S,9R)-N-(4-((Benzo[c][1,2,5]- | | |
| | oxadiazol-5-yloxy)methyl)-5- | | |
| | chloro-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 303^{(d)(j)} | 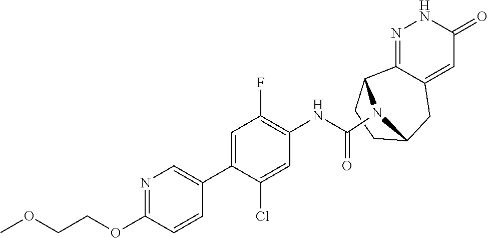 [SMILES: COCCOc1ccc(-c2cc(F)c(NC(=O)N3[C@H]4CC[C@@H]3c3n[nH]c(=O)cc3C4)cc2Cl)cn1] | Method 1 500.2 at 1.13 min | ¹H NMR (400 MHz, DMSO- d6) δ 12.73 (d, J = 2.2 Hz, 1H), 8.89 (s, 1H), 8.20 (dd, J = 2.5, 0.8 Hz, 1H), 7.81 (dd, J = 8.6, 2.5 Hz, 1H), 7.76 (d, J = 7.3 Hz, 1H), 7.38 (d, J = 11.1 Hz, 1H), 6.91 (dd, J = 8.6, 0.7 Hz, 1H), 6.69 (s, 1H), 5.07 (d, J = 5.7 Hz, 1H), 4.68-4.62 (m, 1H), 4.45-4.38 (m, 2H), 3.71-3.64 (m, 2H), 3.30 (s, 3H), 3.25-3.15 (m, 1H), 2.65 (d, J = 18.2 Hz, 1H), 2.25-2.14 (m, 2H), 1.84- |
| | (6S,9R)-N-(5-Chloro-2-fluoro-4- | | 1.75 (m, 1H), 1.72-1.64 |
| | (6-(2-methoxyethoxy)pyridin-3- | | (m, 1H). |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 304^{(d)(j)} | 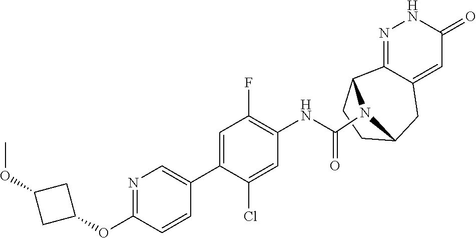 [SMILES: CO[C@H]1C[C@@H](Oc2ccc(-c3cc(F)c(NC(=O)N4[C@H]5CC[C@@H]4c4n[nH]c(=O)cc4C5)cc3Cl)cn2)C1] | Method 1 526.4 at 1.26 min | ¹H NMR (400 MHz, DMSO- d6) δ 12.73 (d, J = 2.3 Hz, 1H), 8.88 (s, 1H), 8.18 (dd, J = 2.5, 0.8 Hz, 1H), 7.80 (dd, J = 8.6, 2.6 Hz, 1H), 7.75 (d, J = 7.4 Hz, 1H), 7.38 (d, J = 11.1 Hz, 1H), 6.88 (dd, J = 8.6, 0.8 Hz, 1H), 6.69 (s, 1H), 5.07 (d, J = 5.6 Hz, 1H), 4.85 (p, J = 7.1 Hz, 1H), 4.64 (s, 1H), 3.65 (p, J = 6.9 Hz, 1H), 3.24-3.16 (m, 1H), 3.16 (s, 3H), 2.91-2.77 (m, 2H), 2.72-2.59 (m, 1H), 2.27- |
| | (6S,9R)-N-(5-Chloro-2-fluoro-4- | | 2.11 (m, 2H), 1.99-1.86 |
| | (6-((1s,3S)-3-methoxy- | | (m, 2H), 1.84-1.75 (m, 1H), |
| | cyclobutoxy)pyridin-3-yl)phenyl)- | | 1.75-1.60 (m, 1H). |
| | 3-oxo-3,5,6,7,8,9-hexahydro-2H- | | |
| | 6,9-epiminocyclohepta- | | |
| | [c]pyridazine-10-carboxamide | | |
| 305^{(d)(j)} | 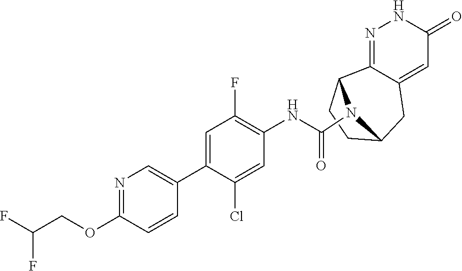 [SMILES: O=C(Nc1cc(Cl)c(-c2ccc(OCC(F)F)nc2)cc1F)N1[C@H]2CC[C@@H]1c1n[nH]c(=O)cc1C2] | Method 1 506.4, 508.3 at 1.30 min | ¹H NMR (400 MHz, DMSO- d6) δ 12.73 (d, J = 2.0 Hz, 1H), 8.90 (s, 1H), 8.24 (dd, J = 2.5, 0.8 Hz, 1H), 7.88 (dd, J = 8.6, 2.5 Hz, 1H), 7.77 (d, J = 7.3 Hz, 1H), 7.39 (d, J = 11.1 Hz, 1H), 7.02 (dd, J = 8.6, 0.8 Hz, 1H), 6.69 (s, 1H), 6.42 (tt, J = 54.7, 3.5 Hz, 1H), 5.08 (d, J = 5.8 Hz, 1H), 4.70-4.53 (m, 3H), 3.25-3.13 (m, 1H), 2.65 (d, J = 18.1 Hz, 1H), 2.28-2.09 (m, 2H), 1.83- 1.75 (m, 1H), 1.74-1.62 (m, 1H). |
| | (6S,9R)-N-(5-Chloro-4-(6-(2,2- | | |
| | difluoroethoxy)pyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 306^{(d)(j)} | 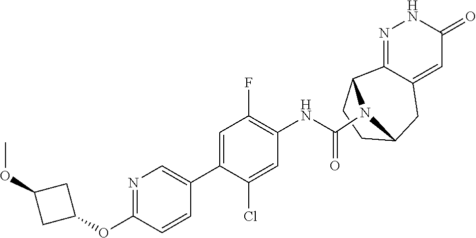 [SMILES: CO[C@H]1C[C@H](Oc2ccc(-c3cc(F)c(NC(=O)N4[C@H]5CC[C@@H]4c4n[nH]c(=O)cc4C5)cc3Cl)cn2)C1] | Method 1 526.3, 528.4 at 1.27 min | ¹H NMR (400 MHz, DMSO- d6) δ 12.73 (d, J = 2.3 Hz, 1H), 8.88 (s, 1H), 8.19 (dd, J = 2.5, 0.8 Hz, 1H), 7.80 (dd, J = 8.6, 2.5 Hz, 1H), 7.75 (d, J = 7.3 Hz, 1H), 7.37 (d, J = 11.1 Hz, 1H), 6.88 (dd, J = 8.6, 0.8 Hz, 1H), 6.69 (s, 1H), 5.28 (tt, J = 7.1, 4.8 Hz, 1H), 5.07 (d, J = 6.0 Hz, 1H), 4.69-4.58 (m, 1H), 4.12-4.02 (m, 1H), 3.25-3.13 (m, 4H), 2.70- 2.58 (m, 1H), 2.46-2.37 (m, 1H), 2.36-2.27 (m, 1H), |
| | (6S,9R)-N-(5-Chloro-2-fluoro-4- | | 2.25-2.12 (m, 2H), 1.84- |
| | (6-((1r,3R)-3- | | 1.74 (m, 1H), 1.74-1.62 |
| | methoxycyclobutoxy)-pyridin-3- | | (m, 1H). |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 307^{(d)(f)} | 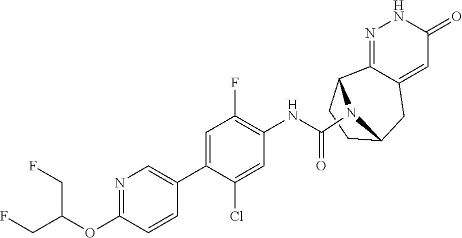 [SMILES: O=C(Nc1cc(Cl)c(-c2ccc(OC(CF)CF)nc2)cc1F)N1[C@H]2CC[C@@H]1c1n[nH]c(=O)cc1C2] | Method 1 519.7, 521.3 at 1.29 min | ¹H NMR (400 MHz, DMSO- d6) δ 12.73 (d, J = 2.3 Hz, 1H), 8.90 (s, 1H), 8.23 (dd, J = 2.5, 0.8 Hz, 1H), 7.87 (dd, J = 8.6, 2.5 Hz, 1H), 7.77 (d, J = 7.3 Hz, 1H), 7.40 (d, J = 11.1 Hz, 1H), 7.00 (dd, J = 8.6, 0.7 Hz, 1H), 6.69 (s, 1H), 5.77- 5.53 (m, 1H), 5.07 (d, J = 5.6 Hz, 1H), 4.88-4.68 (m, 4H), 4.67-4.59 (m, 1H), 3.24-3.15 (m, 1H), 2.65 (d, J = 17.6 Hz, 2H), 2.27-2.13 (m, 2H), 1.83-1.75 (m, 1H), |
| | (6S,9R)-N-(5-Chloro-4-(6-((1,3- | | 1.74-1.63 (m, 1H). |
| | difluoropropan-2-yl)oxy)pyridin-3- | | |
| | yl)-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 308^{(d)(l)} | 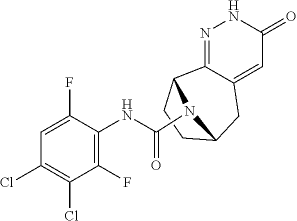 [SMILES: O=C(Nc1c(F)cc(Cl)c(Cl)c1F)N1[C@H]2CC[C@@H]1c1n[nH]c(=O)cc1C2] | Method 1 401.2, 403.2 at 1.41 min | ¹H NMR (400 MHz, DMSO- d6) δ 12.82-12.68 (m, 1H), 8.93 (s, 1H), 7.70 (dd, J = 9.6, 2.1 Hz, 1H), 6.71 (d, J = 1.9 Hz, 1H), 5.01 (d, J = 5.6 Hz, 1H), 4.58 (d, J = 6.4 Hz, 1H), 3.17 (td, J = 9.1, 5.1 Hz, 1H), 2.65 (d, J = 18.1 Hz, 1H), 2.29-2.09 (m, 2H), 1.89-1.74 (m, 1H), 1.74-1.59 (m, 1H). |
| | (6S,9R)-N-(3,4-Dichloro-2,6- | | |
| | difluorophenyl)-3-oxo-3,5,6,7,8,9- | | |
| | hexahydro-2H-6,9- | | |
| | epiminocyclohepta[c]pyridazine- | | |
| | 10-carboxamide | | |
| 310^{(d)(l)} | 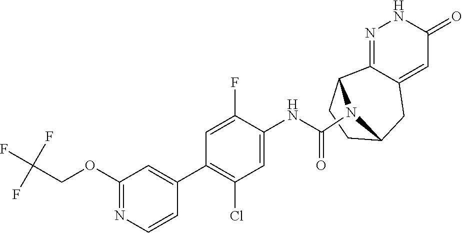 [SMILES: O=C(Nc1cc(Cl)c(-c2ccnc(OCC(F)(F)F)c2)cc1F)N1[C@H]2CC[C@@H]1c1n[nH]c(=O)cc1C2] | Method 1 524.3, 526.1 at 1.45 min | ¹H NMR (400 MHz, DMSO- d6) δ 12.74 (d, J = 2.0 Hz, 1H), 8.95 (s, 1H), 8.28 (dd, J = 5.3, 0.7 Hz, 1H), 7.82 (d, J = 7.2 Hz, 1H), 7.44 (d, J = 11.0 Hz, 1H), 7.21 (dd, J = 5.3, 1.5 Hz, 1H), 7.06 (dd, J = 1.5, 0.8 Hz, 1H), 6.69 (s, 1H), 5.19-4.92 (m, 3H), 4.65 (d, J = 6.3 Hz, 1H), 3.26-3.07 (m, 1H), 2.65 (d, J = 18.2 Hz, 1H), 2.27-2.10 (m, 2H), 1.93-1.73 (m, 1H), 1.68 (dd, J = 10.2, 6.2 Hz, 1H). |
| | (6S,9R)-N-(5-Chloro-2-fluoro-4- | | |
| | (2-(2,2,2-trifluoroethoxy)pyridin-4- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 313^{(d)(l)} | 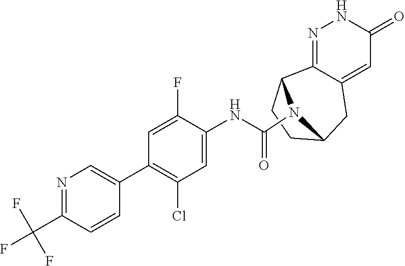 [SMILES: O=C(Nc1cc(Cl)c(-c2ccc(C(F)(F)F)nc2)cc1F)N1[C@H]2CC[C@@H]1c1n[nH]c(=O)cc1C2] | Method 2 494.3, 496.3 at 1.69 min | ¹H NMR (400 MHz, DMSO- d6) δ 12.74 (s, 1H), 8.98 (s, 1H), 8.86 (d, J = 2.2 Hz, 1H), 8.32-8.12 (m, 1H), 8.02 (dd, J = 8.2, 0.8 Hz, 1H), 7.87 (d, J = 7.2 Hz, 1H), 7.55 (d, J = 11.0 Hz, 1H), 6.69 (s, 1H), 5.09 (d, J = 5.6 Hz, 1H), 4.66 (d, J = 6.2 Hz, 1H), 3.26- 3.16 (m, 1H), 2.66 (d, J = 18.2 Hz, 1H), 2.30-2.12 (m, 2H), 1.86-1.75 (m, 1H), 1.74-1.62 (m, 1H). |
| | (6S,9R)-N-(5-Chloro-2-fluoro-4- | | |
| | (6-(trifluoromethyl)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 314^{(d)(h)} | 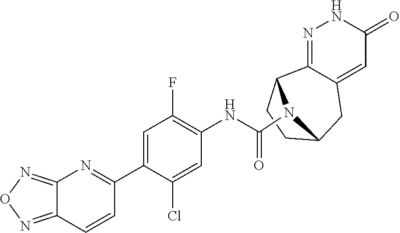 [SMILES: O=C(Nc1cc(Cl)c(-c2ccc3nonc3n2)cc1F)N1[C@H]2CC[C@@H]1c1n[nH]c(=O)cc1C2] | Method 1 468.1, 470.3 at 1.11 min | ¹H NMR (400 MHz, DMSO- d6) δ 12.76-12.71 (m, 1H), 9.07 (s, 1H), 8.69 (d, J = 9.3 Hz, 1H), 7.99 (d, J = 9.4 Hz, 1H), 7.96 (d, J = 7.1 Hz, 1H), 7.68 (d, J = 11.0 Hz, 1H), 6.73-6.65 (m, 1H), 5.11 (d, J = 5.7 Hz, 1H), 4.68 (t, J = 6.0 Hz, 1H), 3.21 (dd, J = 18.3, 5.3 Hz, 1H), 2.67 (d, J = 17.9 Hz, 1H), 2.32-2.13 (m, 2H), 1.86-1.77 (m, 1H), 1.74-1.62 (m, 1H). |
| | (6S,9R)-N-(4-([1,2,5]Oxadiazolo- | | |
| | [3,4-b]pyridin-5-yl)-5-chloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 315^{(d)(h)} | 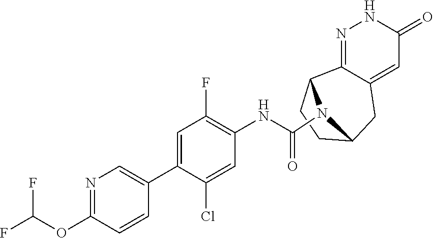 [SMILES: O=C(Nc1cc(Cl)c(-c2ccc(OC(F)F)nc2)cc1F)N1[C@H]2CC[C@@H]1c1n[nH]c(=O)cc1C2] | Method 1 492.4. 494.3 at 1.30 min | ¹H NMR (400 MHz, DMSO- d6) δ 12.72 (s, 1H), 8.91 (s, 1H), 8.33 (dd, J = 2.5, 0.7 Hz, 1H), 8.03 (dd, J = 8.5, 2.5 Hz, 1H), 7.80 (d, J = 7.3 Hz, 1H), 7.77 (t, J = 72.7 Hz, 1H), 7.44 (d, J = 11.0 Hz, 1H), 7.19 (dd, J = 8.5, 0.7 Hz, 1H), 6.69 (s, 1H), 5.08 (d, J = 5.7 Hz, 1H), 4.65 (t, J = 6.2 Hz, 1H), 3.20 (dd, J = 18.2, 5.3 Hz, 1H), 2.70- 2.60 (m, 1H), 2.32-2.11 (m, 2H), 1.88-1.76 (m, 1H), 1.68 (dd, J = 10.2, 6.2 Hz, |
| | (6S,9R)-N-(5-Chloro-4-(6- | | 1H). |
| | (difluoromethoxy)pyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 316^{(d)(p)} | 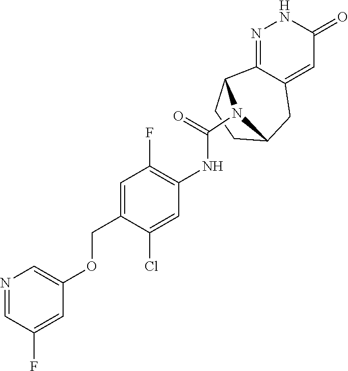 [SMILES: O=C(Nc1cc(Cl)c(COc2cncc(F)c2)cc1F)N1[C@H]2CC[C@@H]1c1n[nH]c(=O)cc1C2] | Method 3 474 at 2.52 min | ¹H NMR (400 MHz, DMSO- d6) δ 12.73 (s, 1H), 9.10 (s, 1H), 8.28 (s, 1H), 8.21 (d, J = 1.6 Hz, 1H), 7.71 (d, J = 7.2 Hz, 1H), 7.59 (d, J = 11.2 Hz, 1H), 7.51 (d, J = 11.2 Hz, 1H), 6.68 (s, 1H), 5.18 (s, 2H), 5.07 (d, J = 1.6 Hz, 1H), 4.69 (t, J = 5.6 Hz, 1H), 3.20 (dd, J = 4.8, 18.4 Hz, 1H), 2.63 (d, J = 18.4 Hz, 1H), 2.24-2.13 (m, 2H), 1.80-1.65 (m, 2H). |
| | (6S,9R)-N-(5-Chloro-2-fluoro-4- | | |
| | (((5-fluoropyridin-3- | | |
| | yl)oxy)methyl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 318^{(d)(j)} | 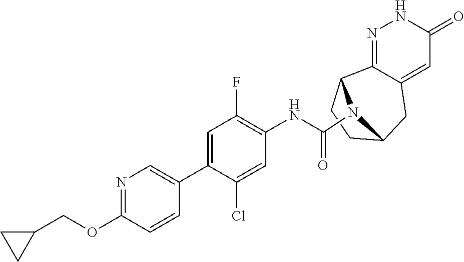 [SMILES: O=C(Nc1cc(Cl)c(-c2ccc(OCC3CC3)nc2)cc1F)N1[C@H]2CC[C@@H]1c1n[nH]c(=O)cc1C2] | Method 1 496.1, 498.3 at 1.41 min | ¹H NMR (400 MHz, DMSO- d6) δ 12.73 (d, J = 2.0 Hz, 1H), 8.88 (s, 1H), 8.19 (dd, J = 2.5, 0.8 Hz, 1H), 7.79 (dd, J = 8.6, 2.6 Hz, 1H), 7.75 (d, J = 7.3 Hz, 1H), 7.37 (d, J = 11.1 Hz, 1H), 6.90 (dd, J = 8.6, 0.8 Hz, 1H), 6.69 (s, 1H), 5.07 (d, J = 5.8 Hz, 1H), 4.69-4.58 (m, 1H), 4.13 (d, J = 7.2 Hz, 2H), 3.19 (dd, J = 18.3, 5.3 Hz, 1H), 2.65 (d, J = 18.4 Hz, 1H), 2.26-2.11 (m, 2H), 1.90-1.75 (m, 1H), 1.68 (dd, J = 10.5, 6.4 Hz, |
| | (6S,9R)-N-(5-Chloro-4-(6- | | 1H), 1.39-1.18 (m, 1H), |
| | (cyclopropylmethoxy)pyridin-3-yl)- | | 0.66-0.50 (m, 2H), 0.41- |
| | 2-fluorophenyl)-3-oxo-3,5,6,7,8,9- | | 0.27 (m, 2H). |
| | hexahydro-2H-6,9- | | |
| | epiminocyclohepta[c]pyridazine- | | |
| | 10-carboxamide | | |
| 319^{(d)(j)} | 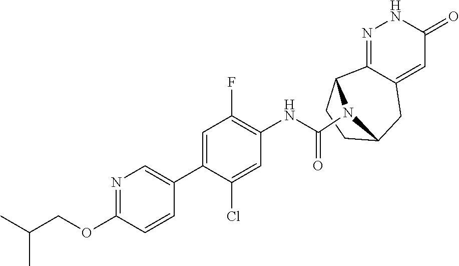 [SMILES: CC(C)COc1ccc(-c2cc(F)c(NC(=O)N3[C@H]4CC[C@@H]3c3n[nH]c(=O)cc3C4)cc2Cl)cn1] | Method 1 498.3, 500.2 at 1.56 min | ¹H NMR (400 MHz, DMSO- d6) δ 12.73 (d, J = 2.1 Hz, 1H), 8.88 (s, 1H), 8.19 (dd, J = 2.5, 0.8 Hz, 1H), 7.79 (dd, J = 8.6, 2.6 Hz, 1H), 7.76 (d, J = 7.4 Hz, 1H), 7.37 (d, J = 11.1 Hz, 1H), 6.89 (dd, J = 8.5, 0.7 Hz, 1H), 6.69 (d, J = 1.9 Hz, 1H), 5.07 (d, J = 5.8 Hz, 1H), 4.64 (t, J = 6.0 Hz, 1H), 4.07 (d, J = 6.6 Hz, 2H), 3.19 (dd, J = 18.3, 5.3 Hz, 1H), 2.65 (d, J = 18.1 Hz, 1H), 2.28- 2.12 (m, 2H), 2.12-1.93 (m, 1H), 1.88-1.74 (m, 1H), 1.74-1.52 (m, 1H), 0.98 (d, |
| | (6S,9R)-N-(5-Chloro-2-fluoro-4- | | J = 6.7 Hz, 6H). |
| | (6-isobutoxypyridin-3-yl)phenyl)- | | |
| | 3-oxo-3,5,6,7,8,9-hexahydro-2H- | | |
| | 6,9-epiminocyclohepta- | | |
| | [c]pyridazine-10-carboxamide | | |
| 320^{(d)(j)} | 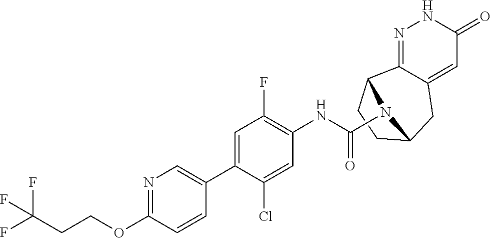 [SMILES: O=C(Nc1cc(Cl)c(-c2ccc(OCCC(F)(F)F)nc2)cc1F)N1[C@H]2CC[C@@H]1c1n[nH]c(=O)cc1C2] | Method 1 538.4, 540.2 at 1.43 min | ¹H NMR (400 MHz, DMSO- d6) δ 12.73 (s, 1H), 8.89 (s, 1H), 8.24 (dd, J = 2.6, 0.8 Hz, 1H), 7.84 (dd, J = 8.6, 2.5 Hz, 1H), 7.77 (d, J = 7.3 Hz, 1H), 7.39 (d, J = 11.1 Hz, 1H), 6.92 (dd, J = 8.6, 0.8 Hz, 1H), 6.69 (s, 1H), 5.07 (d, J = 5.6 Hz, 1H), 4.69-4.58 (m, 1H), 4.53 (t, J = 6.0 Hz, 2H), 3.26-3.10 (m, 1H), 2.95-2.69 (m, 2H), 2.29-2.11 (m, 2H), 1.87- 1.73 (m, 1H), 1.76-1.60 (m, 1H). |
| | (6S,9R)-N-(5-Chloro-2-fluoro-4- | | |
| | (6-(3,3,3-trifluoropropoxy)pyridin- | | |
| | 3-yl)phenyl)-3-oxo-3,5,6,7,8,9- | | |
| | hexahydro-2H-6,9-epiminocyclo- | | |
| | hepta[c]pyridazine-10- | | |
| | carboxamide | | |
| 323^{(d)(l)} | 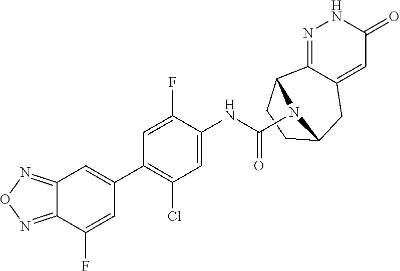 [SMILES: O=C(Nc1cc(Cl)c(-c2cc(F)c3nonc3c2)cc1F)N1[C@H]2CC[C@@H]1c1n[nH]c(=O)cc1C2] | Method 2 485.2, 487.2 at 1.79 min | ¹H NMR (400 MHz, DMSO- d6) δ 12.75 (s, 1H), 9.00 (s, 1H), 8.06 (d, J = 1.0 Hz, 1H), 7.86 (d, J = 7.2 Hz, 1H), 7.69 (dd, J = 11.2, 1.0 Hz, 1H), 7.54 (d, J = 11.0 Hz, 1H), 6.70 (s, 1H), 5.09 (d, J = 5.6 Hz, 1H), 4.66 (t, J = 5.8 Hz, 1H), 3.26-3.17 (m, 1H), 2.66 (d, J = 18.2 Hz, 1H), 2.29-2.03 (m, 2H), 1.93-1.78 (m, 1H), 1.78- 1.59 (m, 1H). |
| | (6S,9R)-N-(5-Chloro-2-fluoro-4- | | |
| | (7-fluorobenzo[c][1,2,5]oxadiazol- | | |
| | 5-yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 326^{(d)(l)} | 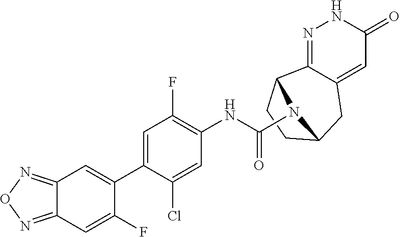 [SMILES: O=C(Nc1cc(Cl)c(-c2cc3nonc3cc2F)cc1F)N1[C@H]2CC[C@@H]1c1n[nH]c(=O)cc1C2] | Method 2 485.3, 487.3 at 1.71 min | ¹H NMR (400 MHz, DMSO- d6) δ 12.73 (s, 1H), 8.99 (s, 1H), 8.31 (dd, J = 6.6, 0.7 Hz, 1H), 8.12 (dd, J = 9.1, 0.7 Hz, 1H), 7.86 (d, J = 7.1 Hz, 1H), 7.55 (d, J = 10.8 Hz, 1H), 6.69 (s, 1H), 5.09 (d, J = 5.6 Hz, 1H), 4.66 (t, J = 6.0 Hz, 1H), 3.26-3.18 (m, 1H), 2.66 (d, J = 17.8 Hz, 1H), 2.29-2.13 (m, 2H), 1.88-1.76 (m, 1H), 1.73-1.65 (m, 1H). |
| | (6S,9R)-N-(5-Chloro-2-fluoro-4- | | |
| | (6-fluorobenzo[c][1,2,5]oxadiazol- | | |
| | 5-yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 327^{(d)(l)} | 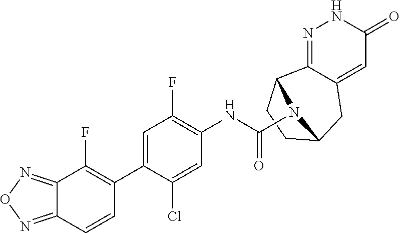 [SMILES: O=C(Nc1cc(Cl)c(-c2ccc3nonc3c2F)cc1F)N1[C@H]2CC[C@@H]1c1n[nH]c(=O)cc1C2] | Method 2 485.3, 487.3 at 1.73 min | ¹H NMR (400 MHz, DMSO- d6) δ 12.73 (d, J = 2.1 Hz, 1H), 9.00 (s, 1H), 8.04 (d, J = 9.2 Hz, 1H), 7.91 (d, J = 7.2 Hz, 1H), 7.64 (dd, J = 9.2, 6.3 Hz, 1H), 7.58 (d, J = 10.9 Hz, 1H), 6.69 (s, 1H), 5.10 (d, J = 5.6 Hz, 1H), 4.66 (t, J = 6.1 Hz, 1H), 3.21 (dd, J = 18.4, 5.6 Hz, 1H), 2.66 (d, J = 18.2 Hz, 1H), 2.29-2.12 (m, 2H), 1.88- 1.77 (m, 1H), 1.74-1.66 (m, 1H). |
| | (6S,9R)-N-(5-Chloro-2-fluoro-4- | | |
| | (4-fluorobenzo[c][1,2,5]oxadiazol- | | |
| | 5-yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 328^{(d)(l)} | 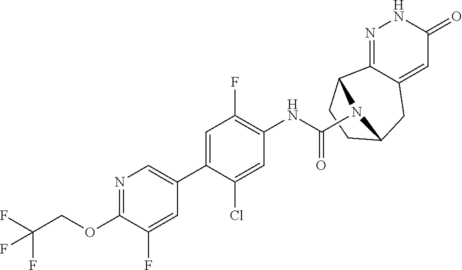 [SMILES: O=C(Nc1cc(Cl)c(-c2cnc(OCC(F)(F)F)c(F)c2)cc1F)N1[C@H]2CC[C@@H]1c1n[nH]c(=O)cc1C2] | Method 2 542.3, 544.4 at 1.88 min | ¹H NMR (400 MHz, DMSO- d6) δ 12.72 (s, 1H), 8.92 (s, 1H), 8.11 (d, J = 2.0 Hz, 1H), 8.00 (dd, J = 11.2, 2.0 Hz, 1H), 7.80 (d, J = 7.3 Hz, 1H), 7.45 (d, J = 11.0 Hz, 1H), 6.69 (s, 1H), 5.15 (q, J = 9.0 Hz, 2H), 5.10-5.05 (m, 1H), 4.64 (t, J = 6.2 Hz, 1H), 3.23-3.16 (m, 1H), 2.71- 2.61 (m, 1H), 2.19 (dd, J = 11.6, 6.4 Hz, 2H), 1.84- 1.77 (m, 1H), 1.73-1.64 (m, 1H). |
| | (6S,9R)-N-(5-Chloro-2-fluoro-4- | | |
| | (5-fluoro-6-(2,2,2- | | |
| | trifluoroethoxy)pyridin-3- | | |
| | yl)phenyl)-3-oxo-3,5,6,7,8,9- | | |
| | hexahydro-2H-6,9- | | |
| | epiminocyclohepta[c]pyridazine- | | |
| | 10-carboxamide | | |
| 331^{(d)(j)} | 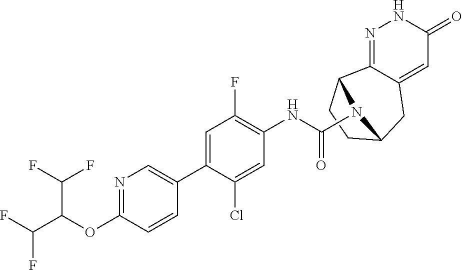 [SMILES: O=C(Nc1cc(Cl)c(-c2ccc(OC(C(F)F)C(F)F)nc2)cc1F)N1[C@H]2CC[C@@H]1c1n[nH]c(=O)cc1C2] | Method 2 556.3, 558.3 at 1.80 min | ¹H NMR (400 MHz, DMSO- d6) δ 12.73 (s, 1H), 8.91 (s, 1H), 8.26 (dd, J = 2.5, 0.8 Hz, 1H), 7.94 (dd, J = 8.6, 2.5 Hz, 1H), 7.78 (d, J = 7.4 Hz, 1H), 7.43 (d, J = 11.1 Hz, 1H), 7.12 (dd, J = 8.6, 0.7 Hz, 1H), 6.69 (s, 1H), 6.67-6.36 (m, 2H), 6.17- 6.00 (m, 1H), 5.08 (d, J = 5.7 Hz, 1H), 4.73-4.58 (m, 1H), 3.24-3.13 (m, 1H), 2.65 (d, J = 17.9 Hz, 1H), 2.29-2.11 (m, 2H), 1.84- 1.75 (m, 1H), 1.73-1.64 (m, 1H). |
| | (6S,9R)-N-(5-Chloro-2-fluoro-4- | | |
| | (6-((1,1,3,3-tetrafluoropropan-2- | | |
| | yl)oxy)pyridin-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 333^{(d)(l)} | 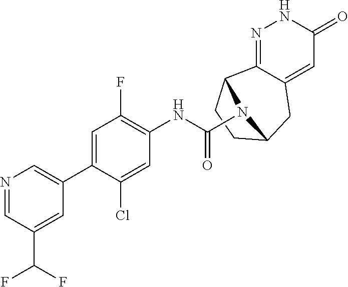 [SMILES: O=C(Nc1cc(Cl)c(-c2cncc(C(F)F)c2)cc1F)N1[C@H]2CC[C@@H]1c1n[nH]c(=O)cc1C2] | Method 1 476.3, 478.3 at 1.04 min | ¹H NMR (400 MHz, DMSO- d6) δ 12.73 (s, 1H), 8.94 (s, 1H), 8.86-8.79 (m, 2H), 8.10 (s, 1H), 7.85 (d, J = 7.2 Hz, 1H), 7.51 (d, J = 11.1 Hz, 1H), 7.22 (t, J = 55.1 Hz, 1H), 6.69 (s, 1H), 5.09 (d, J = 5.8 Hz, 1H), 4.65 (t, J = 7.0 Hz, 1H), 3.27-3.18 (m, 1H), 2.66 (d, J = 18.0 Hz, 1H), 2.31-2.12 (m, 2H), 1.85-1.76 (m, 1H), 1.73- 1.64 (m, 1H) |
| | (6S,9R)-N-(5-Chloro-4-(5- | | |
| | (difluoromethyl)pyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 336^{(d)(l)} | 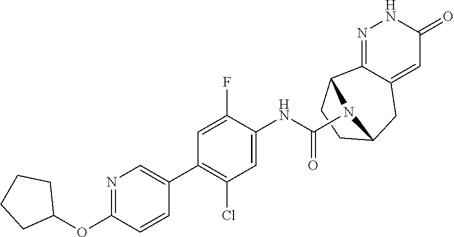 [SMILES: O=C(Nc1cc(Cl)c(-c2ccc(OC3CCCC3)nc2)cc1F)N1[C@H]2CC[C@@H]1c1n[nH]c(=O)cc1C2] | Method 1 510.3, 512.2 at 1.56 min | ¹H NMR (400 MHz, DMSO- d6) δ 12.72 (s, 1H), 8.87 (s, 1H), 8.20 (dd, J = 2.6, 0.8 Hz, 1H), 7.79-7.73 (m, 2H), 7.37 (d, J = 11.1 Hz, 1H), 6.83 (dd, J = 8.6, 0.7 Hz, 1H), 6.69 (s, 1H), 5.40 (dt, J = 6.0, 3.2 Hz, 1H), 5.07 (d, J = 5.8 Hz, 1H), 4.65 (d, J = 6.2 Hz, 1H), 3.20 (d, J = 14.7 Hz, 1H), 2.65 (d, J = 18.0 Hz, 1H), 2.29-2.09 (m, 2H), 2.07- 1.89 (m, 2H), 1.86-1.64 (m, 6H), 1.65-1.51 (m, 2H). |
| | (6S,9R)-N-(5-Chloro-4-(6- | | |
| | (cyclopentyloxy)pyridin-3-yl)-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 337^{(d)(l)} | 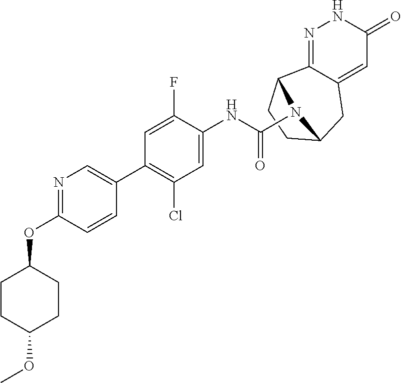 [SMILES: CO[C@H]1CC[C@H](Oc2ccc(-c3cc(F)c(NC(=O)N4[C@H]5CC[C@@H]4c4n[nH]c(=O)cc4C5)cc3Cl)cn2)CC1] | Method 1 554.3, 556.4 at 1.39 min | ¹H NMR (400 MHz, DMSO- d6) δ 12.72 (s, 1H), 8.87 (s, 1H), 8.20 (d, J = 2.6 Hz, 1H), 7.80-7.76 (m, 1H), 7.75 (d, J = 7.5 Hz, 1H), 7.36 (d, J = 11.1 Hz, 1H), 6.84 (d, J = 8.6 Hz, 1H), 6.69 (s, 1H), 5.07 (d, J = 5.9 Hz, 1H), 5.02 (td, J = 9.1, 4.5 Hz, 1H), 4.65 (d, J = 6.3 Hz, 1H), 3.25 (s, 4H), 3.23- 3.12 (m, 1H), 2.65 (d, J = 18.4 Hz, 1H), 2.26-2.13 (m, 2H), 2.10-1.92 (m, 4H), 1.84-1.75 (m, 1H), 1.73- 1.61 (m, 1H), 1.57-1.41 (m, 2H), 1.42-1.29 (m, 2H). |
| | (6S,9R)-N-(5-chloro-2-fluoro-4-(6- | | |
| | (((1r,4r)-4-methoxycyclohexyl)- | | |
| | oxy)pyridin-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 338^{(d)(l)} | 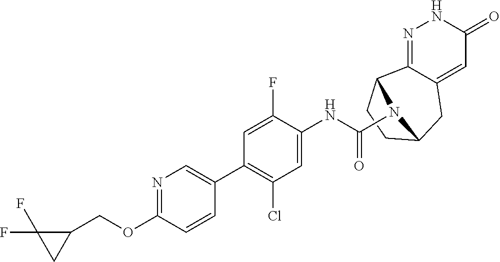 [SMILES: O=C(Nc1cc(Cl)c(-c2ccc(OCC3CC3(F)F)nc2)cc1F)N1[C@H]2CC[C@@H]1c1n[nH]c(=O)cc1C2] | Method 1 532.3, 534.4 at 1.39 min | ¹H NMR (400 MHz, DMSO- d6) δ 12.74 (s, 1H), 8.89 (s, 1H), 8.36-8.13 (m, 1H), 7.83 (dd, J = 8.6, 2.5 Hz, 1H), 7.76 (d, J = 7.4 Hz, 1H), 7.38 (d, J = 11.1 Hz, 1H), 7.04-6.86 (m, 1H), 6.69 (s, 1H), 5.07 (d, J = 5.7 Hz, 1H), 4.64 (s, 1H), 4.50 (ddd, J = 10.4, 6.6, 3.0 Hz, 1H), 4.24 (dd, J = 10.7, 8.3 Hz, 1H), 3.19 (d, J = 14.1 Hz, 1H), 2.65 (d, J = 18.3 Hz, 1H), 2.32-2.05 (m, 3H), 1.87-1.63 (m, 3H), 1.63-1.45 (m, 1H). |
| | (6S,9R)-N-(5-Chloro-4-(6-((2,2- | | |
| | difluorocyclopropyl)methoxy)pyridin- | | |
| | 3-yl)-2-fluorophenyl)-3-oxo- | | |
| | 3,5,6,7,8,9-hexahydro-2H-6,9- | | |
| | epiminocyclohepta[c]pyridazine- | | |
| | 10-carboxamide | | |
| 340^{(d)(i)} | 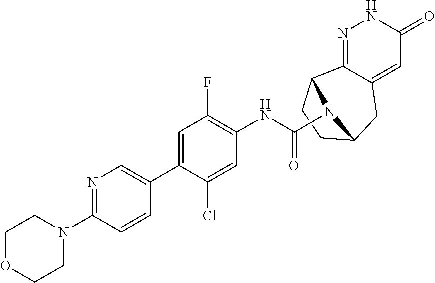 [SMILES: O=C(Nc1cc(Cl)c(-c2ccc(N3CCOCC3)nc2)cc1F)N1[C@H]2CC[C@@H]1c1n[nH]c(=O)cc1C2] | Method 1 511.3, 513.3 at 1.08 min | ¹H NMR (400 MHz, DMSO- d6) δ 12.72 (s, 1H), 8.85 (s, 1H), 8.19 (d, J = 2.5 Hz, 1H), 7.72 (d, J = 7.4 Hz, 1H), 7.67 (dd, J = 8.8, 2.5 Hz, 1H), 7.32 (d, J = 11.1 Hz, 1H), 6.69 (s, 1H), 5.07 (d, J = 5.8 Hz, 1H), 4.64 (t, J = 6.2 Hz, 1H), 3.71 (t, J = 4.8 Hz, 4H), 3.54-3.48 (m, 4H), 3.21 (d, J = 1.8 Hz, 1H), 2.71-2.60 (m, 2H), 2.32- 2.11 (m, 2H), 1.84-1.75 (m, 1H), 1.72-1.60 (m, 1H). |
| | (6S,9R)-N-(5-Chloro-2-fluoro-4- | | |
| | (6-morpholinopyridin-3-yl)phenyl)- | | |
| | 3-oxo-3,5,6,7,8,9-hexahydro-2H- | | |
| | 6,9-epiminocyclohepta- | | |
| | [c]pyridazine-10-carboxamide | | |
| 341^{(d)(i)} | 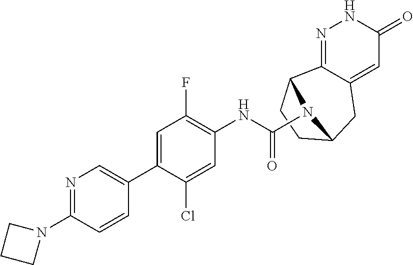 [SMILES: O=C(Nc1cc(Cl)c(-c2ccc(N3CCC3)nc2)cc1F)N1[C@H]2CC[C@@H]1c1n[nH]c(=O)cc1C2] | Method 1 481.2, 483.4 at 1.12 min | ¹H NMR (400 MHz, DMSO- d6) δ 12.72 (s, 1H), 8.83 (s, 1H), 8.11 (dd, J = 2.4, 0.8 Hz, 1H), 7.71 (d, J = 7.4 Hz, 1H), 7.60 (dd, J = 8.6, 2.4 Hz, 1H), 7.29 (d, J = 11.2 Hz, 1H), 6.69 (s, 1H), 6.41 (dd, J = 8.7, 0.8 Hz, 1H), 5.07 (d, J = 5.7 Hz, 1H), 4.63 (t, J = 5.6 Hz, 1H), 3.98 (t, J = 7.4 Hz, 4H), 3.23- 3.17 (m, 1H), 2.65 (d, J = 18.3 Hz, 1H), 2.38-2.29 (m, 2H), 2.24-2.12 (m, 2H), 1.84-1.74 (m, 1H), 1.72- 1.63 (m, 1H). |
| | (6S,9R)-N-(4-(6-(Azetidin-1- | | |
| | yl)pyridin-3-yl)-5-chloro-2- | | |
| | fluorophenyl)-3-oxo-3,5,6,7,8,9- | | |
| | hexahydro-2H-6,9- | | |
| | epiminocyclohepta[c]pyridazine- | | |
| | 10-carboxamide | | |
| 342^{(d)(l)} | 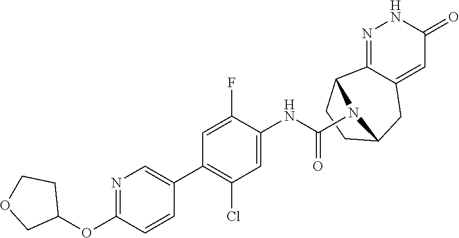 [SMILES: O=C(Nc1cc(Cl)c(-c2ccc(OC3CCOC3)nc2)cc1F)N1[C@H]2CC[C@@H]1c1n[nH]c(=O)cc1C2] | Method 1 512.3, 514.3 at 1.10 min | ¹H NMR (400 MHz, DMSO- d6) δ 12.72 (s, 1H), 8.88 (s, 1H), 8.21 (d, J = 2.5 Hz, 1H), 7.81 (dd, J = 8.6, 2.6 Hz, 1H), 7.76 (d, J = 7.3 Hz, 1H), 7.38 (d, J = 11.1 Hz, 1H), 6.90 (d, J = 8.7 Hz, 1H), 6.69 (s, 1H), 5.54 (ddd, J = 6.7, 4.6, 2.2 Hz, 1H), 5.07 (d, J = 5.8 Hz, 1H), 4.65 (d, J = 6.3 Hz, 1H), 3.97-3.72 (m, 4H), 3.25-3.16 (m, 1H), 2.65 (d, J = 18.1 Hz, 1H), 2.32-2.14 (m, 3H), 2.08- 1.98 (m, 1H), 1.85-1.75 |
| | (6S,9R)-N-(5-Chloro-2-fluoro-4- | | (m, 1H), 1.72-1.64 (m, 1H). |
| | (6-((tetrahydrofuran-3- | | |
| | yl)oxy)pyridin-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |
| 343^{(d)(l)} | 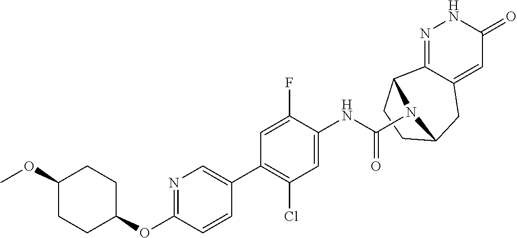 [SMILES: CO[C@H]1CC[C@@H](Oc2ccc(-c3cc(F)c(NC(=O)N4[C@H]5CC[C@@H]4c4n[nH]c(=O)cc4C5)cc3Cl)cn2)CC1] | Method 1 554.4, 556.4 at 1.37 min | ¹H NMR (400 MHz, DMSO- d6) δ 12.72 (d, J = 2.2 Hz, 1H), 8.87 (s, 1H), 8.19 (dd, J = 2.5, 0.7 Hz, 1H), 7.81-7.72 (m, 2H), 7.37 (d, J = 11.1 Hz, 1H), 6.85 (dd, J = 8.6, 0.8 Hz, 1H), 6.69 (s, 1H), 5.07 (d, J = 5.9 Hz, 2H), 4.64 (s, 1H), 3.24 (s, 4H), 3.00 (m, 1H), 2.65 (d, J = 18.3 Hz, 1H), 2.19 (dd, J = 11.5, 6.5 Hz, 2H), 1.85-1.59 (m, 10H). |
| | (6S,9R)-N-(5-Chloro-2-fluoro-4- | | |
| | (6-(((1s,4s)-4-methoxycyclo- | | |
| | hexyl)oxy)pyridin-3-yl)phenyl)-3- | | |
| | oxo-3,5,6,7,8,9-hexahydro-2H- | | |
| | 6,9-epiminocyclohepta- | | |
| | [c]pyridazine-10-carboxamide | | |

^{a}Enamine starting material available from https://www.enaminestore.com/catalog/EN300-33556 (CAS 5211-04-1); see also John Spencer, Rajendra P.Rathnam, Hiren Patel, Nazira Anjum Tetrahedron Volume 64, Issue 44, 27 Oct. 2008, Pages 10195-10200.
^{b}Enamine starting material available from https://www.enaminestore.com/catalog/EN300-1294525; see also Intermediate 4 below.
^{c}ES⁽⁻⁾ reported as ionisation not observed in ES⁽⁺⁾
^{d}DMAP used instead of Et₃N in reaction
^{e}Product purified by column chromatography on silica gel using (3:1 EtOAc:EtOH) in isohexane
^{f}Product purified by column chromatography on silica gel using 0-100% EtOAc/isohexane
^{g}Product purified by column chromatography on silica gel using 0-10% (0.7M NH₃ in MeOH/DCM)
^{(h)}Product purified by column chromatography on reverse phase silica gel using 5-95% MeCN/10 mM Ammonium Bicarbonate
⁽ⁱ⁾Product purified by column chromatography on reverse phase silica gel using 5-100% (0.1% Formic acid in MeCN)/(0.1% Formic acid in water)
^{(j)}Product purified by mass directed reverse phase
^{(k)}Product purified by column chromatography on silica gel using 0-100% (EtOAc/DCM) followed by 0-10% MeOH/EtOAc)
^{(l)}Product purified by column chromatography on silica gel using 0-15% (MeOH/DCM)
^{(m)}Product was purified by tituration with MTBE.
⁽ⁿ⁾Product was purified by prep-TLC (5% MeOH/DCM)
^{(o)}Product was purified by prep-TLC (6% MeOH/DCM)
(p)Product was purified by prep-TLC (10% MeOH/DCM)

### Intermediate 1 (I-1) Route 1

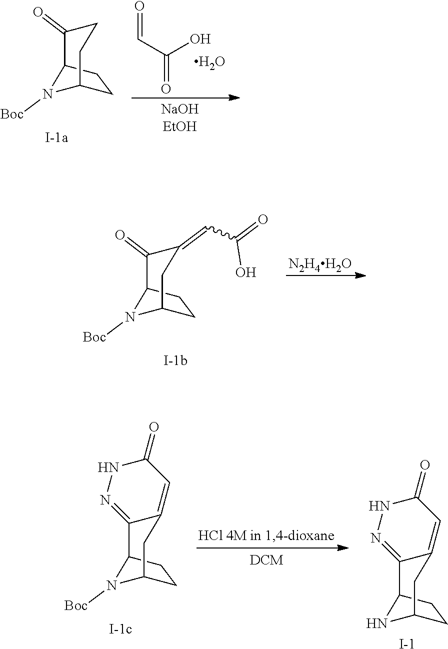

Step 1: tert-Butyl (±)-2-oxo-8-azabicyclo[3.2.1]octane-8-carboxylate I-1a (2.00 g, 8.88 mmol) and glyoxylic acid monohydrate (1.14 g, 12.4 mmol) were added to a round bottom flask, followed by EtOH (20 ml). Once all solids were in solution an aqueous 2 M sodium hydroxide solution (7.10 ml, 14.2 mmol) was added. The resultant mixture was stirred at RT for 1 h. The reaction mixture was concentrated in vacuo. The aqueous was washed once with DCM (5 ml). 1 M HCl was carefully added to the remaining aqueous solution until ˜pH 6 was reached. The product was extracted with DCM (100 ml). The aqueous layer was further extracted with 10% MeOH/DCM (3×50 ml) and the combined organic layers were dried over MgSO₄, filtered and concentrated to provide 2-((±)-8-(tert-butoxycarbonyl)-2-oxo-8-azabicyclo[3.2.1]octa n-3-ylidene)acetic acid I-1b as an off white solid. ¹H NMR (400 MHz, DMSO-d6) δ 6.64-6.54 (m, 1H), 5.75 (s, 1H), 4.35 (d, J=8.1 Hz, 1H), 3.32 (s, 1H), 3.06 (t, J=2.6 Hz, 1H), 2.32-2.16 (m, 1H), 2.16-1.96 (m, 1H), 1.79-1.51 (m, 2H), 1.37 (s, 9H). (Exchangeable—OH not observed)

Step 2: To a solution of 2-((±)-8-(tert-butoxycarbonyl)-2-oxo-8-azabicyclo[3.2.1]octan-3-ylidene)acetic acid I-1b (1.6 g, 5.7 mmol) in EtOH (10 ml) was added hydrazine in THF (23 ml, 1 molar, 23 mmol) was added. The resultant mixture was heated to 78° C. for 16 h. The reaction was cooled to RT and concentrated in vacuo. The resultant solid was dissolved in DCM and passed through a hydrophobic frit to remove residual water. Concentrated in vacuo to give a sticky orange solid. The product was purified by chromatography on silica gel (0-10% MeOH/DCM) to afford tert-butyl (±)-3-hydroxy-6,7,8,9-tetrahydro-5H-6,9-epiminocyclohepta[c]pyridazine-10-carboxylate I-1c as a colourless solid. ¹H NMR (400 MHz, DMSO-d6) δ 6.67 (s, 1H), 4.71 (d, J=6.2 Hz, 1H), 4.31 (br s, 1H), 3.03 (d, J=17.9 Hz, 1H), 2.61 (d, J=18.1 Hz, 1H), 2.22-2.07 (m, 2H), 1.73 (t, J=9.6 Hz, 1H), 1.60 (t, J=8.1 Hz, 1H), 1.35 (s, 9H). (Exchangeable proton not visible in spectrum)

Step 3: tert-butyl (±)-3-hydroxy-6,7,8,9-tetrahydro-5H-6,9-epiminocyclohepta[c]pyridazine-10-carboxylate 1c (471 mg, 1.70 mmol) was dissolved in DCM (4 ml), and a solution of HCl in 1,4-dioxane (4.25 ml, 4 molar, 17.0 mmol) was added. The reaction was stirred at RT for 3 h. The reaction mixture was concentrated in vacuo. The resultant brown solid was dissolved in MeOH and agitated over MP-carbonate resin (2.2 g, 6.84 mmol) for 1 h. The mixture was filtered, and the resin was washed with MeOH (2×10 ml). The filtrate was concentrated in vacuo to give (±)-2,5,6,7,8,9-hexahydro-3H-6,9-epiminocyclohepta[c]pyridazin-3-one I-1 as a brown solid.

### Intermediate 1 (I-1) Route 2

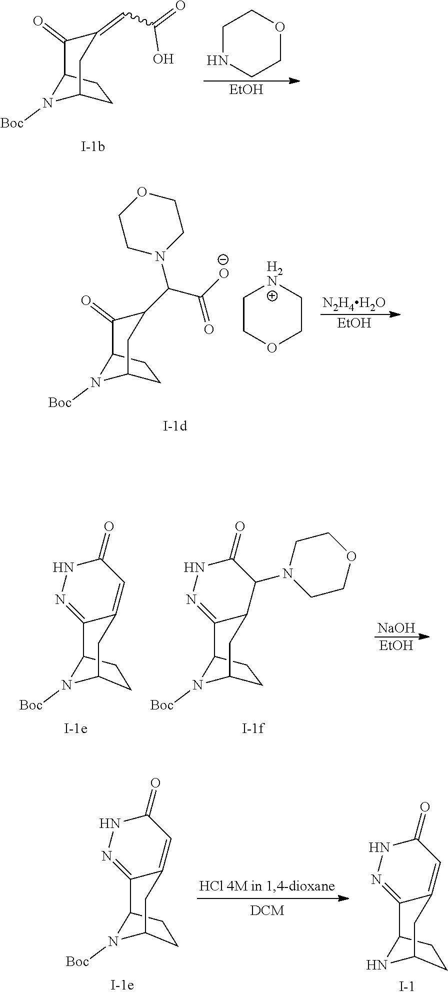

Step 1: To a solution of 2-((±)-8-(tert-butoxycarbonyl)-2-oxo-8-azabicyclo[3.2.1]octan-3-ylidene)acetic acid (4.1 g, 13 mmol) in EtOH (30 ml) at 0° C. was added morpholine (2.3 ml, 27 mmol) was added dropwise. The reaction was stirred at this temperature for 1 h, then allowed to warm to RT and stirred for 72 h. The mixture was concentrated in vacuo to give 2-((±)-8-(tert-butoxycarbonyl)-2-oxo-8-azabicyclo[3.2.1]octan-3-yl)-2-morpholinoacetic acid, morpholine salt I-1d as a sticky yellow oil that was used in the next step without any further purification or analysis.

Step 2: To a solution of 2-((±)-8-(tert-butoxycarbonyl)-2-oxo-8-azabicyclo[3.2.1]octan-3-yl)-2-morpholinoacetic acid, morpholine salt I-1d (17.02 g, 37.37 mmol) in EtOH (120 ml) was added hydrazine monohydrate in water (11.3 ml, 149.5 mmol). The resultant mixture was heated to 78° C. for 2.5 h. The reaction was cooled to RT and the mixture was concentrated in vacuo. The resultant yellow residue was dissolved in DCM (500 ml) and water (150 ml). The layers were separated, and the aqueous layer was extracted with 10% MeOH in DCM (3×200 ml). The combined organic layers were dried over MgSO₄ and concentrated in vacuo to provide a pale yellow solid. The solid was dissolved in the minimum amount of EtOH and allowed to sit at RT for 16 h. A precipitate formed which was filtered to provide tert-butyl (±)-3-oxo-3,5,6,7,8,9-hexahydro-2H-6,9-epiminocyclohepta[c]pyridazine-10-carboxylate I-1e as a white powder. The filtrate was concentrated to provide 50:50 mix of tert-butyl (±)-3-oxo-3,5,6,7,8,9-hexahydro-2H-6,9-epiminocyclohepta[c]pyridazine-10-carboxylate I-1e and tert-butyl (±)-4-morpholino-3-oxo-3,4,4a,5,6,7,8,9-octahydro-2H-6,9-epiminocyclohepta[c]pyridazine-10-carboxylate I-1f as a sticky brown oil.

I-1e: ¹H NMR (400 MHz, DMSO-d6) δ 6.67 (s, 1H), 4.71 (d, J=6.2 Hz, 1H), 4.31 (br s, 1H), 3.03 (d, J=17.9 Hz, 1H), 2.61 (d, J=18.1 Hz, 1H), 2.22-2.07 (m, 2H), 1.73 (t, J=9.6 Hz, 1H), 1.60 (t, J=8.1 Hz, 1H), 1.35 (s, 9H). (Exchangeable proton not visible in spectrum)

I-1f: ¹H NMR (400 MHz, DMSO-d6) δ 10.48 (s, 1H), 4.42 (d, J=6.9 Hz, 1H), 4.22 (d, J=6.7 Hz, 1H), 3.55-3.48 (m, 4H), 3.09-2.92 (m, 4H), 2.69 (dd, J=12.1, 6.0 Hz, 2H), 2.06-2.00 (m, 2H), 1.92 (d, J=10.1 Hz, 1H), 1.69-1.65 (m, 2H), 1.56 (d, J=12.7 Hz, 1H), 1.38 (s, 9H).

Step 3: To a solution of tert-butyl (±)-3-oxo-3,5,6,7,8,9-hexahydro-2H-6,9-epiminocyclohepta[c]pyridazine-10-carboxylate I-1e and tert-butyl (±)-4-morpholino-3-oxo-3,4,4a,5,6,7,8,9-octahydro-2H-6,9-epiminocyclohepta[c]pyridazine-10-carboxylate I-1f (3.2 g, 66% Mol. Wt I-1f) in EtOH (40 ml) was added a 2 M NaOH solution (10 ml, 20 mmol) and the resultant mixture was stirred at RT for 16 h. The reaction mixture was concentrated in vacuo. The aqueous was neutralised to pH 7 using 1 M HCl. The product was extracted with 10% MeOH in DCM (3×150 ml). The combined organics were dried with MgSO₄. The solvent was removed in vacuo to give tert-butyl (±)-3-oxo-3,5,6,7,8,9-hexahydro-2H-6,9-epiminocyclohepta[c]pyridazine-10-carboxylate I-1e as a white solid.

Step 4: To a solution of tert-butyl (±)-3-oxo-3,5,6,7,8,9-hexahydro-2H-6,9-epiminocyclohepta[c]pyridazine-10-carboxylate (2.6 g, 9.4 mmol) in DCM (20 ml) was added a solution of HCl in 1,4-dioxane (23 ml, 4 M, 94 mmol) was added. The reaction was stirred at RT for 72 h. The reaction mixture was concentrated in vacuo to give the HCl salt of I-1. The HCl salt was dissolved in MeOH (150 ml), AcOH was added and the solution was loaded onto SCX resin (50 g). The cartridge was washed with MeOH and the product was eluted with 0.7 M NH₃ in MeOH solution. To give (±)-2,5,6,7,8,9-hexahydro-3H-6,9-epiminocyclohepta[c]pyridazin-3-one I-1 as an off white solid.

Alternatively the HCl was dissolved in MeOH and MP-carbonate (3 eq.) was added and the mixture was left for 1 h. The mixture was filtered. The resin was washed with MeOH. The filtrate was concentrated in vacuo to give (±)-2,5,6,7,8,9-hexahydro-3H-6,9-epiminocyclohepta[c]pyridazin-3-one I-1 as an off white solid.

### Intermediate 2 (I-2)

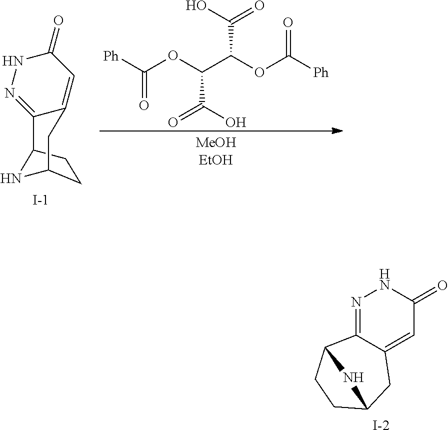

To a solution of (±)-2,5,6,7,8,9-hexahydro-3H-6,9-epiminocyclohepta[c]pyridazin-3-one (200 mg, 1.13 mmol) in MeOH (10 ml) was added a solution of dibenzoyl-L-tartaric acid in MeOH (5 ml). The solvent was removed in vacuo and the residue was redissolved in EtOH (40 ml), heated to 60° C. for 1 h and allowed to cool and left standing for 2 h. The solid was filtered off and dried in vacuo. The solid was dissolved in MeOH (10 ml) and MP-carbonate (600 mg, 1.8 mmol) was added and the mixture was left to stand for 1 h. The mixture was filtered and the filtrate was concentrated in vacuo to give (5R,8S)-3,5,6,7,8,9-hexahydro-2H-5,8-epiminocyclohepta[d]pyrimidin-2-one as an off white solid. ¹H NMR (400 MHz, DMSO-d6) δ 12.48 (s, 1H), 6.56 (s, 1H), 4.05-3.99 (m, 1H), 3.63 (t, J=5.9 Hz, 1H), 2.90-2.78 (m, 1H), 2.45 (t, J=1.4 Hz, 1H), 1.98-1.81 (m, 2H), 1.65 (t, J=9.3 Hz, 1H), 1.53-1.39 (m, 1H). (NH not observed)

### Intermediate 4 (I-4)

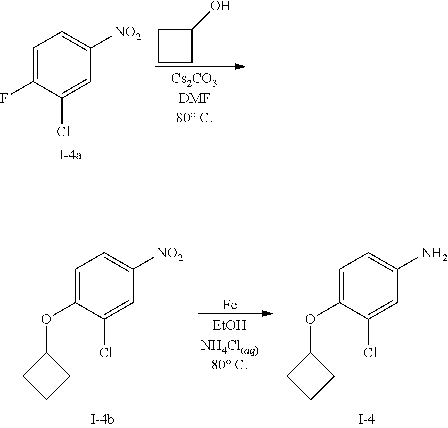

Step 1: To a mixture of 2-chloro-1-fluoro-4-nitrobenzene I-4a (1 g, 5.70 mmol) and cyclobutanol (1.2 g, 17.09 mmol) in DMF (30 ml) was added Cs₂CO₃ (6.5 g, 19.94 mmol). The reaction was heated at 80° C. for 16 h, then concentrated in vacuo. The product was purified by silica gel chromatography (5% EtOAc/petroleum ether) to give 2-chloro-1-cyclobutoxy-4-nitrobenzene I-4b as a yellow oil. ¹HNMR: (400 MHz, DMSO-d6) δ 8.30-8.29 (m, 1H), 8.19-8.16 (m, 1H), 7.19 (d, J=9.2 Hz, 1H), 4.97-4.90 (m, 1H), 2.49-2.50 (m, 2H), 2.16-2.06 (m, 2H), 1.87-1.79 (m, 1H), 1.73-1.62 (m, 1H).

Step 2: To a solution of 2-chloro-1-cyclobutoxy-4-nitrobenzene I-4b (500 mg, 2.20 mmol) in a mixture of EtOH and saturated aqueous NH₄Cl solution (12 ml, 1:1) was added Iron powder (614 mg, 10.98 mmol). The reaction was heated at 80° C. for 2 h, cooled, then concentrated in vacuo. The product was purified by silica gel chromatography (5% EtOAc/petroleum ether) to give 3-chloro-4-cyclobutoxyaniline I-4 as a yellow oil. ¹HNMR (400 MHz, DMSO-d6) δ 6.70-6.68 (m, 1H), 6.62 (d, J=2.8 Hz, 1H), 6.45-6.42 (m, 1H), 4.87 (s, 2H), 4.53-4.46 (m, 1H), 2.35-2.27 (m, 2H). 2.05-1.95 (m, 2H), 1.76-1.51 (m, 2H).

### Intermediate 16 (I-16)

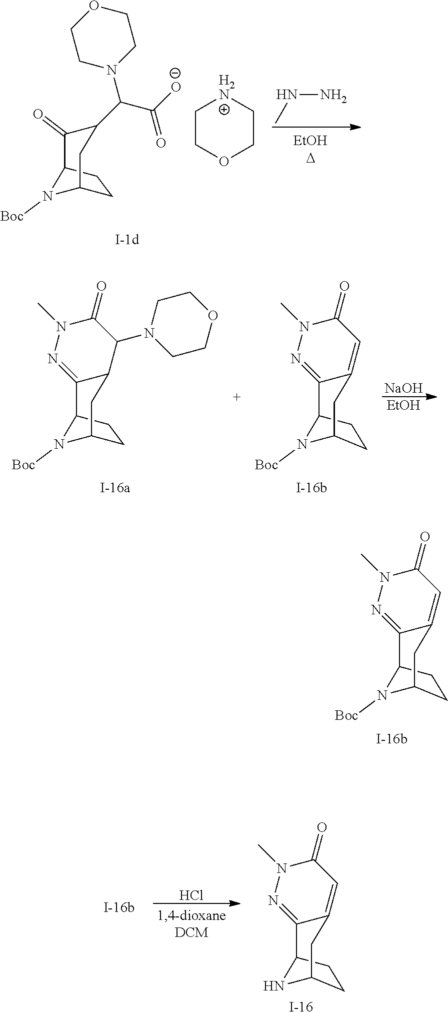

Step 1: To a suspension of 2-((±)-8-(tert-butoxycarbonyl)-2-oxo-8-azabicyclo[3.2.1]octan-3-yl)-2-morpholinoacetic acid, morpholine salt I-1d (490 mg, 1.08 mmol) in EtOH (5.0 ml) was added methylhydrazine (229 μl, 4.30 mmol) and the resulting solution was stirred at 80° C. for 2 h, then at RT for 16 h. The solvent was removed in vacuo. The residue was taken up in DCM (20 ml) and washed with 1 M HCl (20 ml). The aqueous layer was extracted with 10% MeOH in DCM (2×15 ml) and the combined organics were dried over MgSO₄ and concentrated in vacuo to afford a mixture of tert-butyl (±)-2-methyl-3-oxo-3,5,6,7,8,9-hexahydro-2H-6,9-epiminocyclohepta[c]pyridazine-10-carboxylate I-16b and tert-butyl (±)-2-methyl-4-morpholino-3-oxo-3,4,4a,5,6,7,8,9-octahydro-2H-6,9-epiminocyclohepta[c]pyridazine-10-carboxylate I-16a as a viscous yellow oil. The mixture was carried forward to the next step without any further purification.

Step 2: A mixture of tert-butyl (±)-2-methyl-3-oxo-3,5,6,7,8,9-hexahydro-2H-6,9-epiminocyclohepta[c]pyridazine-10-carboxylate I-16b and tert-butyl (±)-2-methyl-4-morpholino-3-oxo-3,4,4a,5,6,7,8,9-octahydro-2H-6,9-epiminocyclohepta[c]pyridazine-10-carboxylate I-16a (315 mg, 1.08 mmol) in EtOH (10.0 ml) was added to a vial charged with sodium hydroxide (300 mg, 7.5 mmol) and the mixture was stirred at RT for 1 h. The mixture was concentrated in vacuo. The residue was taken up in water and acidified to pH 2 with careful addition of 1 M HCl. DCM (20 ml) was added and the layers were separated. The aqueous layer was further extracted with DCM (2×15 ml) and the combined organics were washed with water (20 ml), dried over MgSO₄ and concentrated in vacuo to afford tert-butyl (±)-2-methyl-3-oxo-3,5,6,7,8,9-hexahydro-2H-6,9-epiminocyclohepta[c]pyridazine-10-carboxylate I-16b as an orange oil. ¹H NMR (400 MHz, DMSO-d6) δ 6.74 (s, 1H), 4.79-4.70 (m, 1H), 4.43-4.25 (m, 1H), 3.58 (s, 3H), 3.03 (dd, J=18.1, 5.4 Hz, 1H), 2.63 (d, J=18.1 Hz, 1H), 2.22-2.06 (m, 2H), 1.79-1.71 (m, 1H), 1.65-1.55 (m, 1H), 1.36 (s, 9H).

Step 3: To a solution of tert-butyl (±)-2-methyl-3-oxo-3,5,6,7,8,9-hexahydro-2H-6,9-epiminocyclohepta[c]pyridazine-10-carboxylate I-16b (280 mg, 702 μmol) in DCM (7 ml) was added HCl in dioxane (3.0 ml, 4.0 molar, 12 mmol) and the resulting mixture was stirred at RT for 4 h. The mixture was concentrated in vacuo and the residue was taken up in MeOH and loaded onto SCX (˜3 g). The SCX was washed with MeOH, and the product was eluted with 0.7 M NH₃ in MeOH to afford (±)-2-methyl-2,5,6,7,8,9-hexahydro-3H-6,9-epiminocyclohepta[c]pyridazin-3-one as a yellow oil which solidified upon standing to give a yellow solid. ¹H NMR (400 MHz, DMSO-d6) δ 6.63 (t, J=1.7 Hz, 1H), 4.07-4.01 (m, 1H), 3.67-3.60 (m, 1H), 3.56 (s, 3H), 2.90-2.81 (m, 1H), 2.52-2.43 (m, 1H), 1.99-1.83 (m, 2H), 1.73-1.64 (m, 1H), 1.49-1.40 (m, 1H). Exchangeable proton not observed.

### Intermediate 32 (I-32)

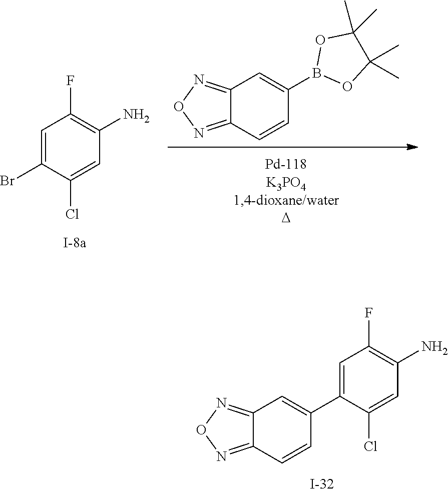

To a vial was added 4-bromo-5-chloro-2-fluoroaniline I-8a (316 mg, 1.41 mmol), 5-(4,4,5,5-tetramethyl-1,3,2-dioxaborolan-2-yl)benzo[c][1,2,5]oxadiazole (346 mg, 1.41 mmol) and Pd-118 (22.9 mg, 35.2 μmol). The vial was evacuated under vacuum and back filled with N₂. This was repeated 3 times. 1,4-Dioxane (4.00 ml) was added, followed by an aqueous solution of tripotassium phosphate (2.11 ml, 2.00 M, 4.22 mmol) which had been sparged with N₂. After addition, the vial was once more evacuated and backfilled with N₂. The reaction was heated to 95° C. for 2 h. The reaction was cooled to RT and filtered through a pad of Celite, washing with DCM (20 ml). The filtrate was diluted with water (4 ml) and the layers were separated. The aqueous phase was extracted with DCM (3×10 ml). The combined organics were dried over MgSO4, filtered and concentrated in vacuo. The product was purified by chromatography on silica gel (0-30% EtOAc/isohexane) to afford 4-(benzo[c][1,2,5]oxadiazol-5-yl)-5-chloro-2-fluoroaniline I-32 as a bright yellow solid. ¹H NMR (400 MHz, DMSO-d6) δ 8.06 (dd, J=9.3, 1.0 Hz, 1H), 8.00 (t, J=1.3 Hz, 1H), 7.68 (dd, J=9.3, 1.4 Hz, 1H), 7.31 (d, J=11.9 Hz, 1H), 6.95 (d, J=8.2 Hz, 1H), 5.81 (s, 2H).

### Intermediate 33 (I-33)

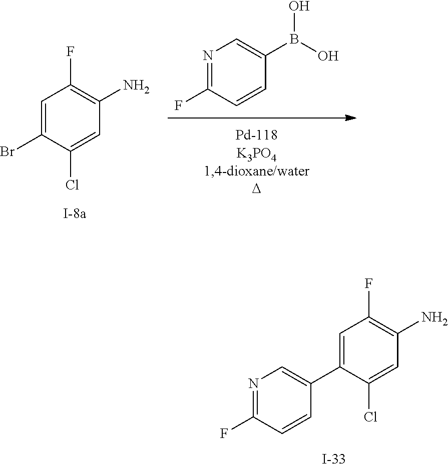

5-Chloro-2-fluoro-4-(5-fluoropyridin-3-yl)aniline I-33 was synthesized from 4-bromo-5-chloro-2-fluoroaniline I-8a and (6-fluoropyridin-3-yl)boronic acid using a procedure essentially the same as for I-32. ¹H NMR (400 MHz, DMSO-d6) δ 8.23 (d, J=2.6 Hz, 1H), 8.02 (td, J=8.2, 2.6 Hz, 1H), 7.23 (dd, J=8.5, 2.9 Hz, 1H), 7.20 (d, J=11.9 Hz, 1H), 6.93 (d, J=8.3 Hz, 1H), 5.68 (s, 2H).

### Intermediate 34 (I-34)

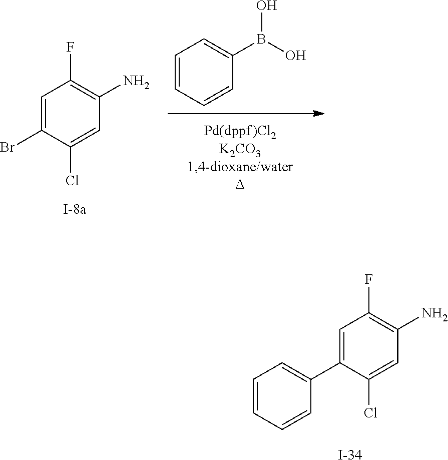

To a solution of 4-bromo-5-chloro-2-fluoroaniline (100 mg, 446 μmol) and 4-bromo-5-chloro-2-fluoroaniline I-8a (100 mg, 446 μmol) in 1,4-dioxane (5 ml) was added K₂CO₃ (185 mg, 1.34 mmol) in water (1 ml). The mixture was purged with N₂ for 10 mins and Pd(dppf)Cl₂ (16.3 mg, 22.3 μmol) was added. The reaction mixture was heated to 80° C. for 2 h. The reaction mixture was concentrated in vacuo. The product was purified by chromatography on silica gel (0-2% 0.7 M NH₃/MeOH in DCM) to give 2-chloro-5-fluoro-[1,1′-biphenyl]-4-amine I-34 as a colourless oil. ¹H NMR (400 MHz, DMSO-d6) δ 7.45-7.30 (m, 5H), 7.06 (d, J=12.0 Hz, 1H), 6.90 (d, J=8.4 Hz, 1H), 5.55 (s, 2H).

### Intermediate 35 (I-35)

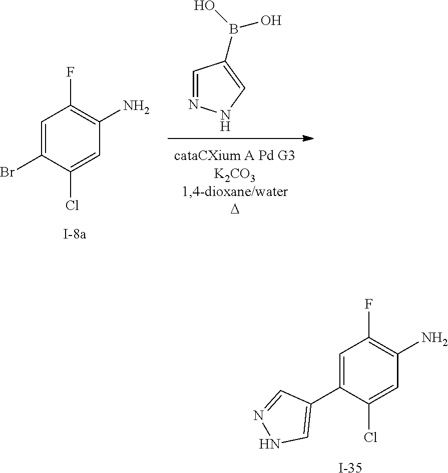

To a vial containing cataCXium® A Pd G3 (3.2 mg, 4.5 μmol) was added (1H-pyrazol-4-yl)boronic acid (29.9 mg, 267 μmol) and 4-bromo-5-chloro-2-fluoroaniline I-8a (20.0 mg, 89.1 μmol) in 1,4-dioxane (1.00 ml) and a solution of K₂CO₃ in water (208 μl, 1.5 M, 312 μmol). The mixture was stirred at 95° C. for 2 h. The reaction mixture was loaded onto SCX (1 g), washed with MeOH and the product was eluted with 0.7 M ammonia in MeOH to afford 5-chloro-2-fluoro-4-(1H-pyrazol-4-yl)aniline I-35. LCMS (Method 1) m/z 212.2, 214.2 (M+H)⁺ (ES⁺), at 0.85 min.

### Intermediate 36 (I-36)

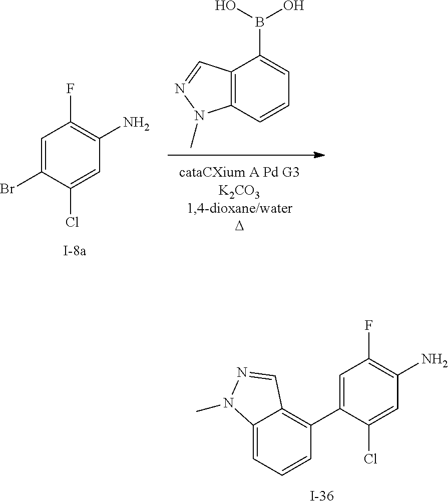

5-Chloro-2-fluoro-4-(1-methyl-1H-indazol-4-yl)aniline I-34 was synthesised from 4-bromo-5-chloro-2-fluoroaniline I-8a using a procedure essentially the same as for I-35. LCMS (Method 1) m/z 275.8 (M+H)⁺ (ES⁺), at 1.40 min

### Intermediate 37 (I-37)

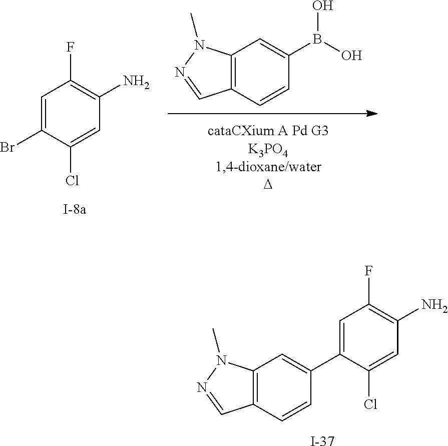

A vial containing (1-methyl-1H-indazol-6-yl)boronic acid (31 mg, 178 μmol) was purged with N₂. A solution of 4-bromo-5-chloro-2-fluoroaniline I-8a (20.0 mg, 89.1 μmol) and cataCXium A Pd G3 (3.2 mg, 4.45 μmol) in 1,4-dioxane (1.00 ml) and a solution of 1.5 M tripotassium phosphate in water (119 μl, 1.5 M, 178 μmol). The mixture was stirred at 95° C. for 2 h. The reaction mixture was loaded onto SCX (1 g), washed with MeOH and the product was eluted with 0.7 M ammonia in MeOH to give 5-chloro-2-fluoro-4-(1-methyl-1H-indazol-6-yl)aniline. LCMS (Method 1) m/z 275.7, 278.4 (M+H)⁺ (ES⁺), at 1.37 min

### Intermediate 38 (I-38)

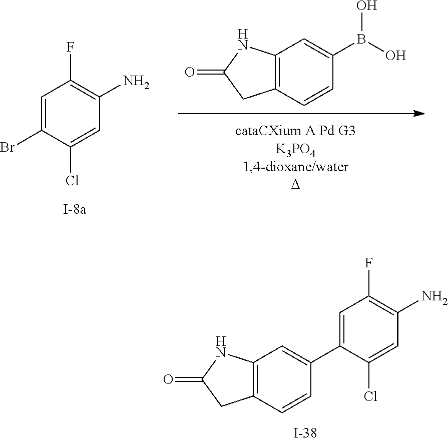

6-(4-Amino-2-chloro-5-fluorophenyl)indolin-2-one I-38 was synthesised from 4-bromo-5-chloro-2-fluoroaniline I-8a and (2-oxoindolin-6-yl)boronic acid using a procedure essentially the same as for I-37. LCMS (Method 1) m/z 276.2, 278.4 (M+H)⁺ (ES⁺), at 1.37 min

### Intermediate 39 (I-39)

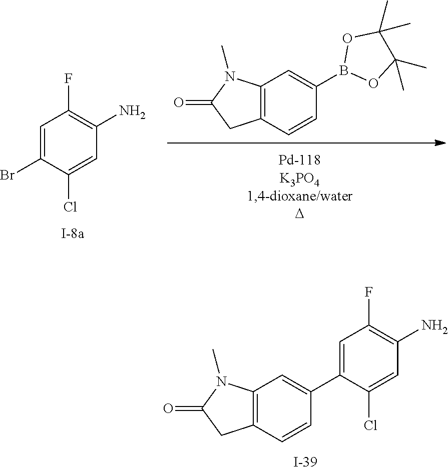

6-(4-Amino-2-chloro-5-fluorophenyl)-1-methylindolin-2-one I-39 was synthesised from 4-bromo-5-chloro-2-fluoroaniline I-8a and 1-methyl-6-(4,4,5,5-tetramethyl-1,3,2-dioxaborolan-2-yl)indolin-2-one using a procedure essentially the same as for I-32. ¹H NMR (400 MHz, DMSO-d6) δ 7.27 (d, J=7.6 Hz, 1H), 7.09 (d, J=11.9 Hz, 1H), 7.01 (dd, J=7.6, 1.6 Hz, 1H), 6.95 (d, J=1.5 Hz, 1H), 6.90 (d, J=8.4 Hz, 1H), 5.56 (s, 2H), 3.56 (d, J=1.1 Hz, 2H), 3.13 (s, 3H).

### Intermediate 40 (I-40)

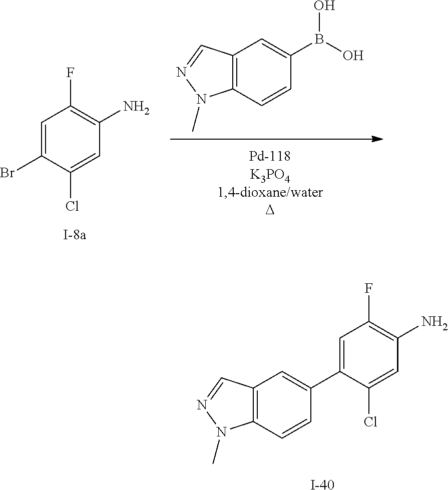

5-Chloro-2-fluoro-4-(1-methyl-1H-indazol-5-yl)aniline I-40 was synthesised from 4-bromo-5-chloro-2-fluoroaniline I-8a and (1-methyl-1H-indazol-5-yl)boronic acid using a procedure essentially the same as for I-32. ¹H NMR (400 MHz, DMSO-d6) δ 8.05 (d, J=0.9 Hz, 1H), 7.73-7.68 (m, 1H), 7.67-7.62 (m, 1H), 7.40 (dd, J=8.7, 1.6 Hz, 1H), 7.10 (d, J=11.9 Hz, 1H), 6.91 (d, J=8.4 Hz, 1H), 5.52 (s, 2H), 4.06 (s, 3H).

### Intermediate 41 (I-41)

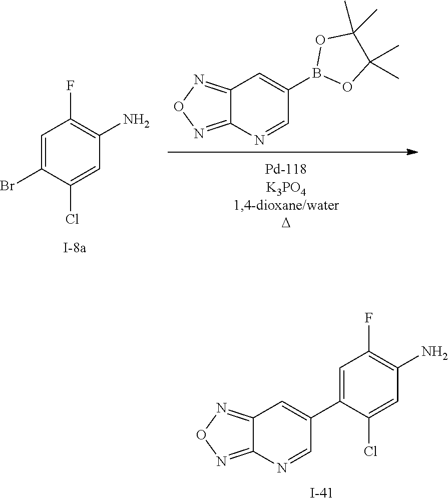

4-([1,2,5]Oxadiazolo[3,4-b]pyridin-6-yl)-5-chloro-2-fluoroaniline I-41 was synthesised from 4-bromo-5-chloro-2-fluoroaniline I-8a and 6-(4,4,5,5-tetramethyl-1,3,2-dioxaborolan-2-yl)-[1,2,5]oxadiazolo[3,4-b]pyridine using a procedure essentially the same as for I-32. ¹H NMR (400 MHz, DMSO-d6) δ 9.15 (d, J=2.1 Hz, 1H), 8.55 (d, J=2.1 Hz, 1H), 7.42 (d, J=11.9 Hz, 1H), 6.98 (d, J=8.2 Hz, 1H), 5.95 (s, 2H).

### Intermediate 42 (I-42)

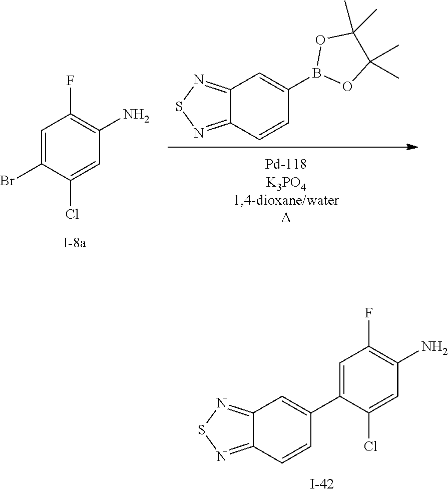

4-(Benzo[c][1,2,5]thiadiazol-5-yl)-5-chloro-2-fluoroaniline I-42 was synthesised from 4-bromo-5-chloro-2-fluoroaniline I-8a and 5-(4,4,5,5-tetramethyl-1,3,2-dioxaborolan-2-yl)benzo[c][1,2,5]thiadiazole using a procedure essentially the same as for I-32. ¹H NMR (400 MHz, DMSO-d6) δ 8.10 (dd, J=9.1, 0.8 Hz, 1H), 8.05 (dd, J=1.8, 0.8 Hz, 1H), 7.78 (dd, J=9.0, 1.8 Hz, 1H), 7.29 (d, J=11.9 Hz, 1H), 6.96 (d, J=8.3 Hz, 1H), 5.73 (s, 2H)

### Intermediate 43 (I-43)

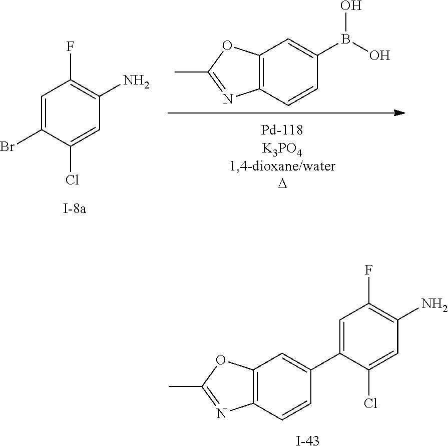

5-Chloro-2-fluoro-4-(2-methyl-3a,7a-dihydrobenzo[d]oxazol-6-yl)aniline I-43 was synthesised from 4-bromo-5-chloro-2-fluoroaniline I-8a and (2-methylbenzo[d]oxazol-6-yl)boronic acid using a procedure essentially the same as for I-32. ¹H NMR (400 MHz, DMSO-d6) δ 7.69-7.62 (m, 2H), 7.32 (dd, J=8.3, 1.6 Hz, 1H), 7.13 (d, J=12.0 Hz, 1H), 6.92 (d, J=8.4 Hz, 1H), 5.59 (s, 2H), 2.62 (s, 3H).

### Intermediate 44 (I-44)

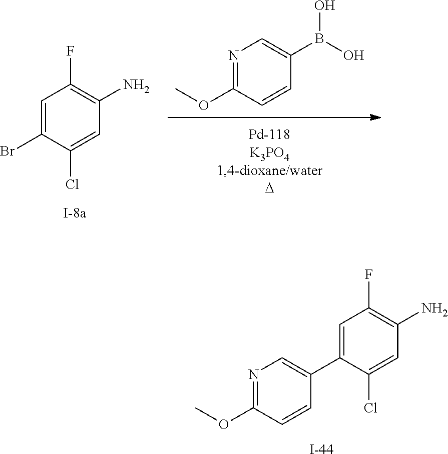

5-Chloro-2-fluoro-4-(6-methoxypyridin-3-yl)aniline I-44 was synthesised from 4-bromo-5-chloro-2-fluoroaniline I-8a and (6-methoxypyridin-3-yl)boronic acid using a procedure essentially the same as for I-32. ¹H NMR (400 MHz, DMSO-d6) δ 8.15 (dd, J=2.6, 0.7 Hz, 1H), 7.73 (dd, J=8.6, 2.5 Hz, 1H), 7.12 (d, J=11.9 Hz, 1H), 6.91 (d, J=8.3 Hz, 1H), 6.85 (dd, J=8.6, 0.8 Hz, 1H), 5.58 (s, 2H), 3.88 (s, 3H).

### Intermediate 45 (I-45)

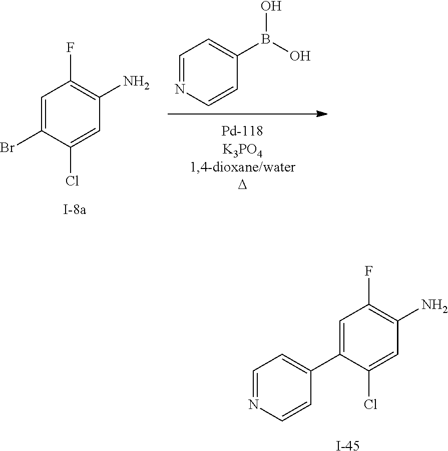

5-Chloro-2-fluoro-4-(pyridin-4-yl)aniline I-45 was synthesised from 4-bromo-5-chloro-2-fluoroaniline I-8a and pyridin-4-ylboronic acid using a procedure essentially the same as for 1-32. ¹H NMR (400 MHz, DMSO-d6) δ 8.62-8.56 (m, 2H), 7.46-7.40 (m, 2H), 7.19 (d, J=12.0 Hz, 1H), 6.92 (d, J=8.3 Hz, 1H), 5.76 (s, 2H).

### Intermediate 46 (I-46)

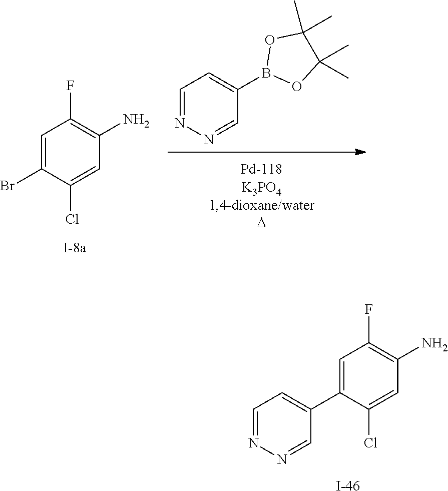

5-Chloro-2-fluoro-4-(pyridazin-4-yl)aniline I-46 was synthesised from 4-bromo-5-chloro-2-fluoroaniline I-8a and 4-(4,4,5,5-tetramethyl-1,3,2-dioxaborolan-2-yl)pyridazine using a procedure essentially the same as for I-32. ¹H NMR (400 MHz, DMSO-d6) δ 9.31 (dd, J=2.5, 1.2 Hz, 1H), 9.24 (dd, J=5.4, 1.2 Hz, 1H), 7.76 (dd, J=5.4, 2.4 Hz, 1H), 7.36 (d, J=12.0 Hz, 1H), 6.95 (d, J=8.2 Hz, 1H), 5.92 (s, 2H).

### Intermediate 47 (I-47)

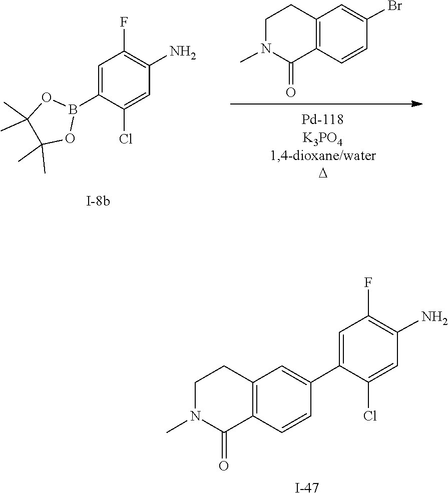

6-bromo-2-methyl-3,4-dihydroisoquinolin-1-one (141 mg, 589 μmol), 5-chloro-2-fluoro-4-(4,4,5,5-tetramethyl-1,3,2-dioxaborolan-2-yl)aniline I-8b (160 mg, 589 μmol) and Pd-118 (10.0 mg, 15.3 μmol) were added to a scintillation vial. The vial was flushed with N₂. 1,4-Dioxane (3.00 ml) was added, followed by a degassed aqueous solution of potassium phosphate (884 μl, 2 M, 1.77 mmol). The reaction was heated to 95° C. for 2 h. The reaction was cooled to RT and water (15 ml) was added followed by extraction with DCM (3×10 ml). The combined organics were passed through a hydrophobic frit and concentrated in vacuo. The product was purified by chromatography on silica gel (10-30% EtOAc/DCM) to afford 6-(4-amino-2-chloro-5-fluorophenyl)-2-methyl-3,4-dihydroisoquinolin-1(2H)-one I-47 as a light grey solid. ¹H NMR (400 MHz, DMSO-d6) δ 7.87 (d, J=8.0 Hz, 1H), 7.35 (dd, J=8.0, 1.8 Hz, 1H), 7.30 (d, J=1.7 Hz, 1H), 7.11 (d, J=11.9 Hz, 1H), 6.91 (d, J=8.3 Hz, 1H), 5.63 (s, 2H), 3.56 (t, J=6.7 Hz, 2H), 3.03 (s, 3H), 3.00 (t, J=6.7 Hz, 2H).

### Intermediate 49 (I-49)

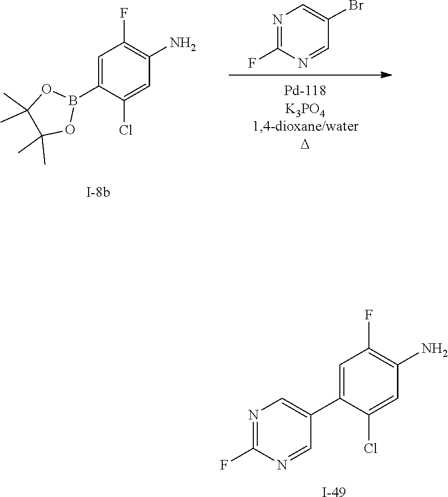

5-Chloro-2-fluoro-4-(2-fluoropyrimidin-5-yl)aniline I-49 was synthesised from 5-chloro-2-fluoro-4-(4,4,5,5-tetramethyl-1,3,2-dioxaborolan-2-yl)aniline I-8b and 5-bromo-2-fluoropyrimidine using a procedure essentially the same as for I-47. ¹H NMR (400 MHz, DMSO-d6) δ 8.85 (d, J=1.6 Hz, 2H), 7.32 (d, J=11.9 Hz, 1H), 6.95 (d, J=8.2 Hz, 1H), 5.80 (s, 2H).

### Intermediate 50 (I-50)

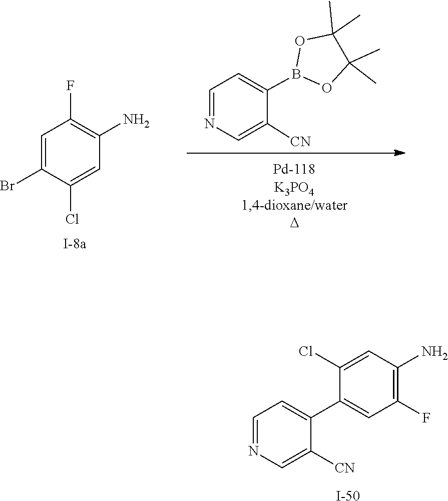

4-(4-Amino-2-chloro-5-fluorophenyl)nicotinonitrile I-50 was synthesised from 4-bromo-5-chloro-2-fluoroaniline I-8a and 4-(4,4,5,5-tetramethyl-1,3,2-dioxaborolan-2-yl)nicotinonitrile using a procedure essentially the same as for I-32. ¹H NMR (400 MHz, DMSO-d6) δ 9.08 (d, J=0.7 Hz, 1H), 8.87 (d, J=5.2 Hz, 1H), 7.59 (dd, J=5.2, 0.8 Hz, 1H), 7.28 (d, J=11.6 Hz, 1H), 6.96 (d, J=8.1 Hz, 1H), 5.93 (s, 2H).

### Intermediate 51 (I-51)

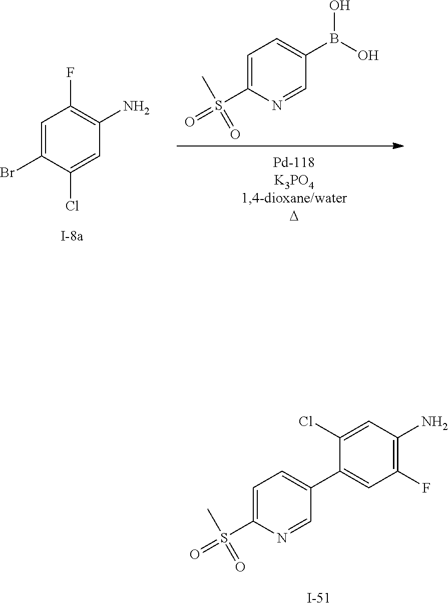

5-Chloro-2-fluoro-4-(6-(methylsulfonyl)pyridin-3-yl)aniline I-50 was synthesised from 4-bromo-5-chloro-2-fluoroaniline I-8a and (6-(methylsulfonyl)pyridin-3-yl)boronic acid using a procedure essentially the same as for I-32. ¹H NMR (400 MHz, DMSO-d6) δ 8.81 (dt, J=2.7, 1.3 Hz, 1H), 8.19 (dd, J=8.1, 2.2 Hz, 1H), 8.08 (dd, J=8.2, 0.8 Hz, 1H), 7.31 (d, J=11.9 Hz, 1H), 6.96 (d, J=8.2 Hz, 1H), 5.84 (s, 2H).

### Intermediate 52 (I-52)

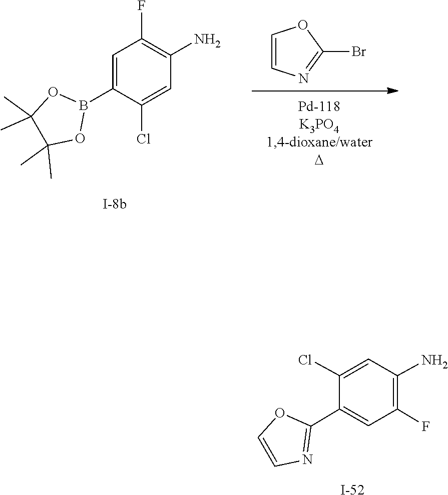

5-Chloro-2-fluoro-4-(oxazol-2-yl)aniline I-52 was synthesised from 5-chloro-2-fluoro-4-(4,4,5,5-tetramethyl-1,3,2-dioxaborolan-2-yl)aniline I-8b and 2-bromooxazole using a procedure essentially the same as for I-47. ¹H NMR (400 MHz, DMSO-d6) δ 8.17 (d, J=0.9 Hz, 1H), 7.56 (d, J=12.1 Hz, 1H), 7.33 (d, J=0.8 Hz, 1H), 6.89 (d, J=8.1 Hz, 1H), 6.06 (s, 2H).

### Intermediate 53 (I-53)

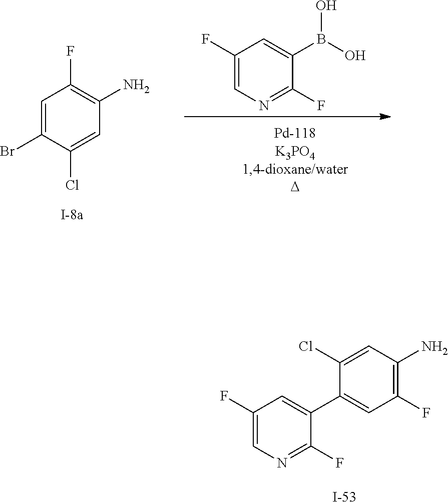

5-Chloro-4-(2,5-difluoropyridin-3-yl)-2-fluoroaniline I-53 was synthesised from 4-bromo-5-chloro-2-fluoroaniline I-8a and (2,5-difluoropyridin-3-yl)boronic acid using a procedure essentially the same as for I-32. ¹H NMR (400 MHz, DMSO-d6) δ 8.27 (dd, J=3.1, 1.8 Hz, 1H), 7.98 (td, J=7.8, 3.0 Hz, 1H), 7.23 (d, J=11.7 Hz, 1H), 6.92 (d, J=8.2 Hz, 1H), 5.79 (s, 2H).

### Intermediate 54 (I-54)

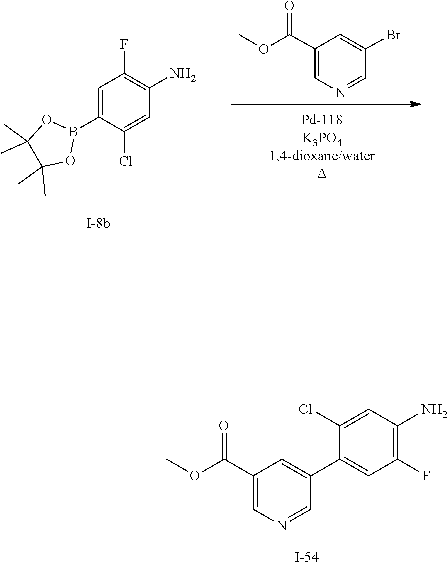

Methyl 5-(4-amino-2-chloro-5-fluorophenyl)nicotinate I-54 was synthesised from 5-chloro-2-fluoro-4-(4,4,5,5-tetramethyl-1,3,2-dioxaborolan-2-yl)aniline I-8b and methyl 5-bromonicotinate using a procedure essentially the same as for I-47. ¹H NMR (400 MHz, DMSO-d6) δ 9.04 (d, J=2.0 Hz, 1H), 8.83 (d, J=2.3 Hz, 1H), 8.27 (t, J=2.1 Hz, 1H), 7.28 (d, J=11.9 Hz, 1H), 6.95 (d, J=8.3 Hz, 1H), 5.75 (s, 2H), 3.91 (s, 3H)

### Intermediate 55 (I-55)

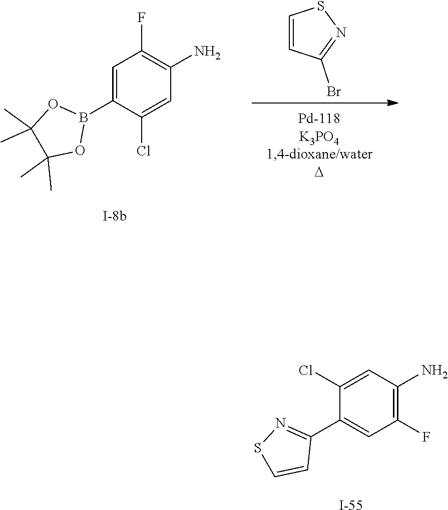

5-Chloro-2-fluoro-4-(isothiazol-3-yl)aniline I-55 was synthesised from 5-chloro-2-fluoro-4-(4,4,5,5-tetramethyl-1,3,2-dioxaborolan-2-yl)aniline I-8b and 3-bromoisothiazole using a procedure essentially the same as for I-47. ¹H NMR (400 MHz, DMSO-d6) δ 9.11 (d, J=4.7 Hz, 1H), 7.70 (d, J=4.7 Hz, 1H), 7.41 (d, J=12.2 Hz, 1H), 6.89 (d, J=8.2 Hz, 1H), 5.81 (s, 2H).

### Intermediate 56 (I-56)

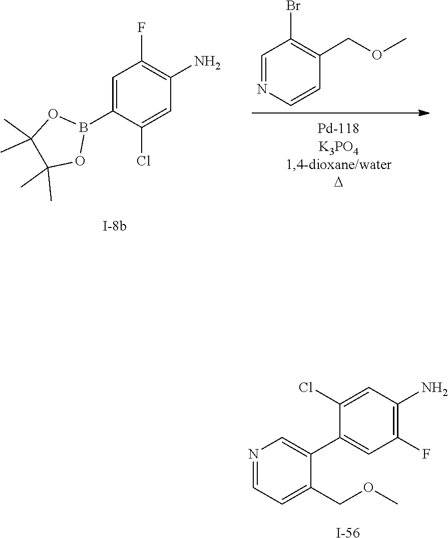

5-Chloro-2-fluoro-4-(2-(methoxymethyl)pyridine-4-yl)aniline I-56 was synthesised from 5-chloro-2-fluoro-4-(4,4,5,5-tetramethyl-1,3,2-dioxaborolan-2-yl)aniline I-8b and 3-bromo-4-(methoxymethyl)pyridine using a procedure essentially the same as for I-47. ¹H NMR (400 MHz, DMSO-d6) δ 8.55 (d, J=5.1 Hz, 1H), 8.30 (d, J=0.8 Hz, 1H), 7.46 (dd, J=5.1, 0.9 Hz, 1H), 7.06 (d, J=11.6 Hz, 1H), 6.92 (d, J=8.3 Hz, 1H), 5.65 (s, 2H), 4.23 (d, J=3.1 Hz 2H), 3.25 (s, 3H).

### Intermediate 57 (I-57)

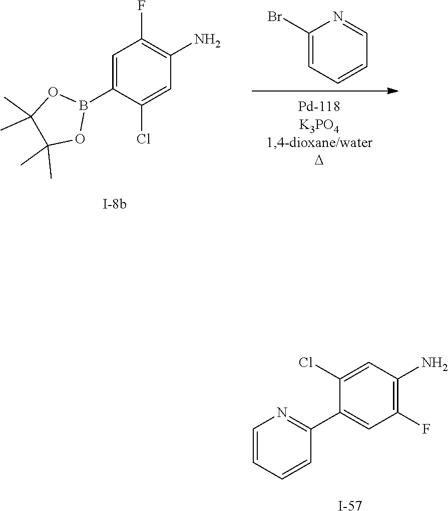

5-Chloro-2-fluoro-4-(pyridin-2-yl)aniline I-57 was synthesised from 5-chloro-2-fluoro-4-(4,4,5,5-tetramethyl-1,3,2-dioxaborolan-2-yl)aniline I-8b and 2-bromopyridine using a procedure essentially the same as for I-47. ¹H NMR (400 MHz, DMSO-d6) δ 8.62 (ddd, J=4.8, 1.8, 1.0 Hz, 1H), 7.83 (td, J=7.7, 1.9 Hz, 1H), 7.65 (dt, J=7.9, 1.1 Hz, 1H), 7.32 (ddd, J=7.5, 4.9, 1.1 Hz, 1H), 7.28 (d, J=12.2 Hz, 1H), 6.89 (d, J=8.3 Hz, 1H), 5.70 (s, 2H).

### Intermediate 58 (I-58)

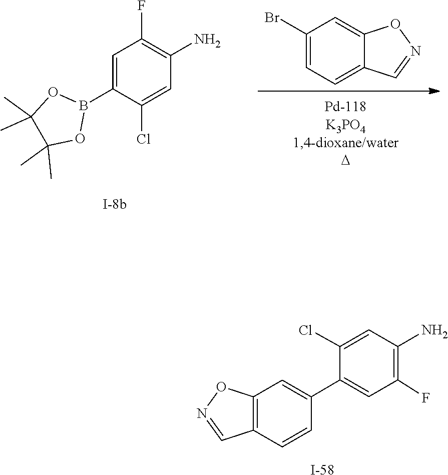

4-(Benzo[d]isoxazol-6-yl)-5-chloro-2-fluoroaniline I-58 was synthesised from 5-chloro-2-fluoro-4-(4,4,5,5-tetramethyl-1,3,2-dioxaborolan-2-yl)aniline I-8b and 6-bromobenzo[d]isoxazole using a procedure essentially the same as for I-47. ¹H NMR (400 MHz, DMSO-d6) δ 11.06 (s, 1H), 7.61 (d, J=8.1 Hz, 1H), 7.09 (d, J=11.9 Hz, 1H), 7.01 (t, J=2.2 Hz, 1H), 6.95-6.87 (m, 2H), 5.69 (s, 2H).

### Intermediate 59 (I-59)

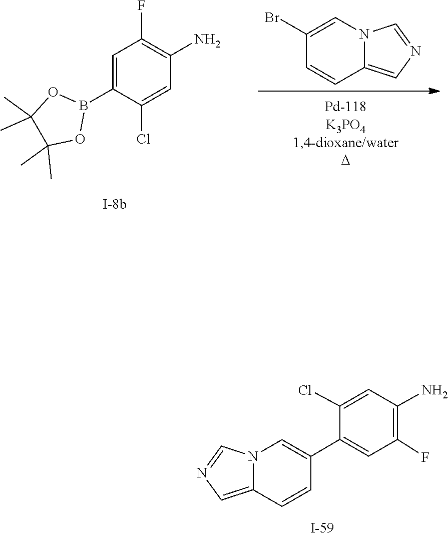

5-Chloro-2-fluoro-4-(imidazo[1,5-a]pyridine-6-yl)aniline I-59 was synthesised from 5-chloro-2-fluoro-4-(4,4,5,5-tetramethyl-1,3,2-dioxaborolan-2-yl)aniline I-8b and 6-bromoimidazo[1,5-a]pyridine using a procedure essentially the same as for I-47. ¹H NMR (400 MHz, DMSO-d6) δ 8.41-8.32 (m, 2H), 7.55 (dt, J=9.4, 1.0 Hz, 1H), 7.37 (s, 1H), 7.19 (d, J=11.9 Hz, 1H), 6.92 (d, J=8.3 Hz, 1H), 6.81 (dd, J=9.4, 1.5 Hz, 1H), 5.63 (s, 2H).

### Intermediate 60 (I-60)

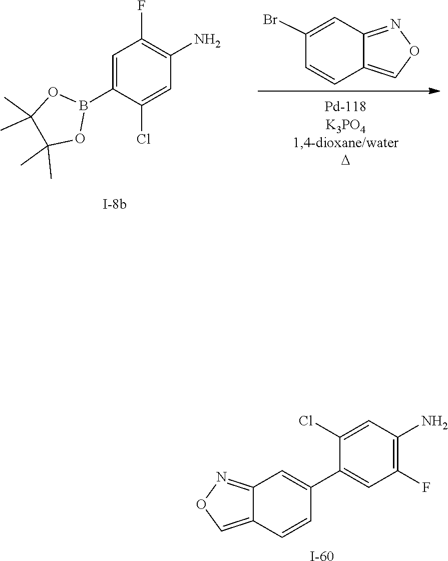

4-(Benzo[c]isoxazole-6-yl)-5-chloro-2-fluoroaniline I-60 was synthesised from 5-chloro-2-fluoro-4-(4,4,5,5-tetramethyl-1,3,2-dioxaborolan-2-yl)aniline I-8b and 6-bromobenzo[c]isoxazole using a procedure essentially the same as for I-47. ¹H NMR (400 MHz, CDCl₃) δ 9.14 (s, 1H), 7.62-7.52 (m, 2H), 7.11 (dd, J=8.9, 1.4 Hz, 1H), 7.06 (d, J=11.2 Hz, 1H), 6.90 (d, J=8.2 Hz, 1H).

### Intermediate 61 (I-61)

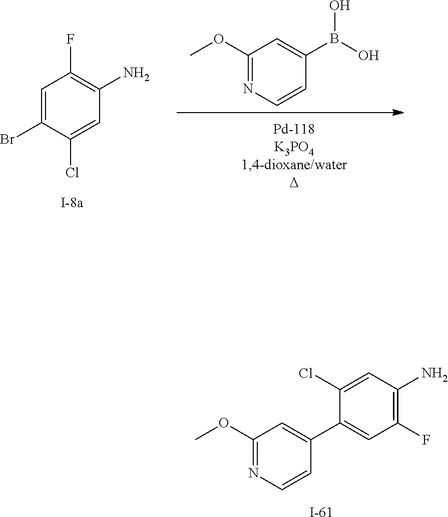

5-Chloro-2-fluoro-4-(2-methoxypyridin-4-yl)aniline I-61 was synthesised from 4-bromo-5-chloro-2-fluoroaniline I-8a and (2-methoxypyridin-4-yl)boronic acid using a procedure essentially the same as for I-32. ¹H NMR (400 MHz, DMSO-d6) δ 8.17 (dd, J=5.3, 0.7 Hz, 1H), 7.16 (d, J=12.0 Hz, 1H), 7.02 (dd, J=5.3, 1.5 Hz, 1H), 6.90 (d, J=8.3 Hz, 1H), 6.81 (dd, J=1.5, 0.7 Hz, 1H), 5.74 (s, 2H), 3.87 (s, 3H).

### Intermediate 63 (I-63)

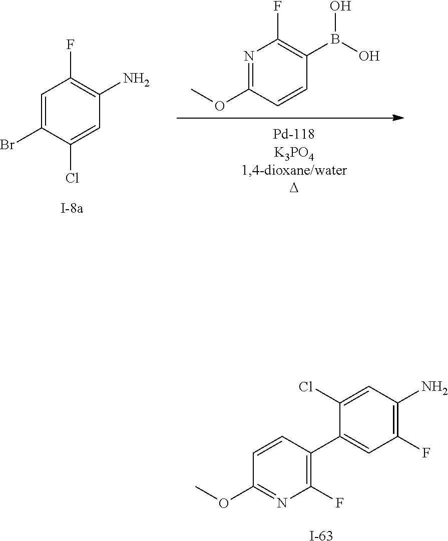

5-Chloro-2-fluoro-4-(2-fluoro-6-methoxypyridin-3-yl)aniline I-63 was synthesised from 4-bromo-5-chloro-2-fluoroaniline I-8a and (2-fluoro-6-methoxypyridin-3-yl)boronic acid using a procedure essentially the same as for I-32. ¹H NMR (400 MHz, DMSO-d6) δ 7.79 (dd, J=10.0, 8.2 Hz, 1H), 7.10 (d, J=11.8 Hz, 1H), 6.91 (d, J=8.3 Hz, 1H), 6.82 (dd, J=8.2, 1.2 Hz, 1H), 5.64 (s, 2H), 3.87 (s, 3H).

### Intermediate 64 (I-64)

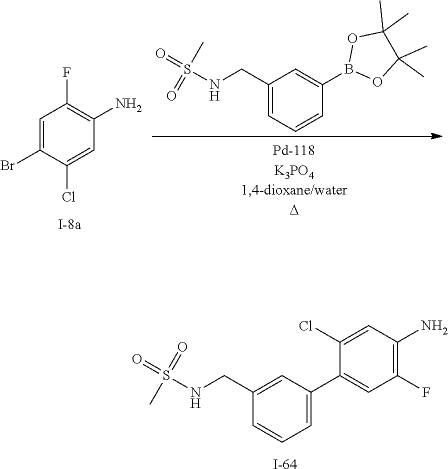

N-((4′-Amino-2′-chloro-5′-fluoro-[1,1′-biphenyl]-3-yl)methyl)methanesulfonamide I-63 was synthesised from 4-bromo-5-chloro-2-fluoroaniline I-8a and N-(3-(4,4,5,5-tetramethyl-1,3,2-dioxaborolan-2-yl)benzyl)methanesulfonamide using a procedure essentially the same as for I-32. ¹H NMR (400 MHz, DMSO-d6) δ 7.57 (t, J=6.4 Hz, 1H), 7.38 (d, J=7.5 Hz, 1H), 7.35 (t, J=1.7 Hz, 1H), 7.30 (ddt, J=7.2, 3.3, 1.7 Hz, 2H), 7.11-7.00 (m, 1H), 6.90 (d, J=8.4 Hz, 1H), 5.57 (s, 2H), 4.19 (d, J=6.3 Hz, 2H), 2.86 (s, 3H).

### Intermediate 66 (I-66)

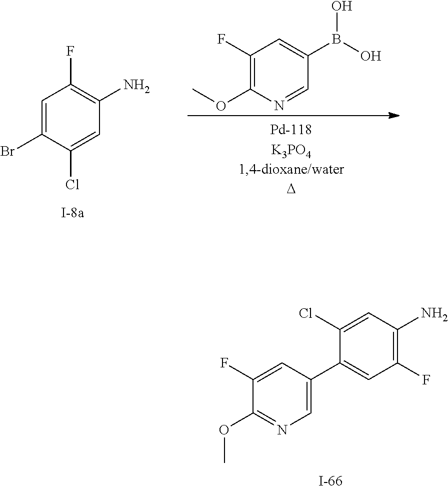

5-Chloro-2-fluoro-4-(5-fluoro-6-methoxypyridin-3-yl)aniline I-66 was synthesised from 4-bromo-5-chloro-2-fluoroaniline I-8a and (5-fluoro-6-methoxypyridin-3-yl)boronic acid using a procedure essentially the same as for I-32. ¹H NMR (400 MHz, DMSO-d6) δ 8.17 (d, J=3.0 Hz, 1H), 7.62 (dd, J=8.5, 3.0 Hz, 1H), 7.08 (d, J=11.8 Hz, 1H), 6.97-6.81 (m, 1H), 5.62 (s, 2H), 3.82 (s, 3H).

### Intermediate 67 (I-67)

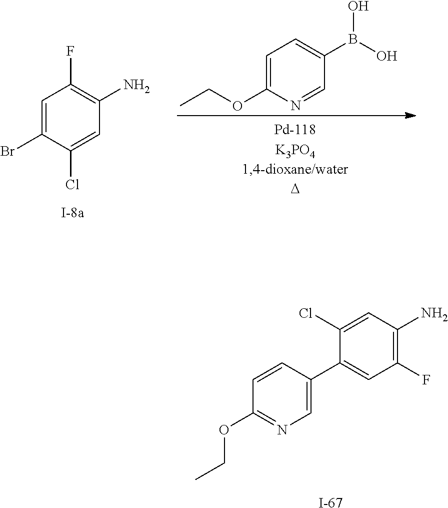

5-Chloro-4-(6-ethoxypyridin-3-yl)-2-fluoroaniline I-67 was synthesised from 4-bromo-5-chloro-2-fluoroaniline I-8a and (6-ethoxypyridin-3-yl)boronic acid using a procedure essentially the same as for I-32. ¹H NMR (400 MHz, DMSO-d6) δ 8.14 (dd, J=2.5, 0.7 Hz, 1H), 7.72 (dd, J=8.6, 2.6 Hz, 1H), 7.12 (d, J=11.9 Hz, 1H), 6.91 (d, J=8.4 Hz, 1H), 6.83 (dd, J=8.5, 0.7 Hz, 1H), 5.58 (s, 2H), 4.33 (q, J=7.0 Hz, 2H), 1.34 (t, J=7.0 Hz, 3H).

### Intermediate 68 (I-68)

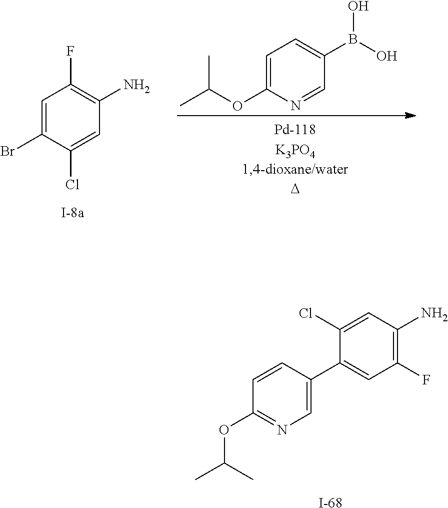

5-Chloro-2-fluoro-4-(6-isopropoxypyridin-3-yl)aniline I-68 was synthesised from 4-bromo-5-chloro-2-fluoroaniline I-8a and (6-isopropoxypyridin-3-yl)boronic acid using a procedure essentially the same as for I-32. ¹H NMR (400 MHz, DMSO-d6) δ 8.13 (dd, J=2.6, 0.8 Hz, 1H), 7.70 (dd, J=8.6, 2.6 Hz, 1H), 7.12 (d, J=11.9 Hz, 1H), 6.91 (d, J=8.4 Hz, 1H), 6.77 (dd, J=8.6, 0.7 Hz, 1H), 5.58 (s, 2H), 5.27 (hept, J=6.2 Hz, 1H), 1.31 (d, J=6.2 Hz, 6H).

### Intermediate 69 (I-69)

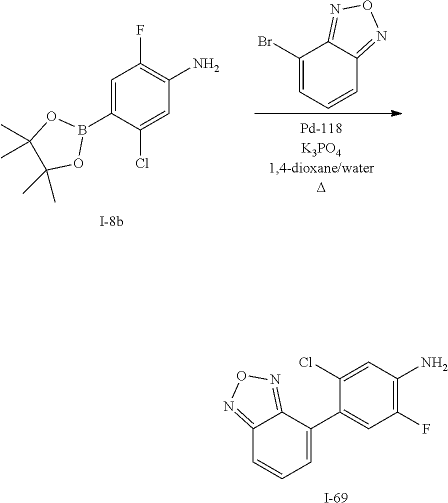

4-(Benzo[c][1,2,5]oxadiazol-4-yl)-5-chloro-2-fluoroaniline I-69 was synthesised from 5-chloro-2-fluoro-4-(4,4,5,5-tetramethyl-1,3,2-dioxaborolan-2-yl)aniline I-8b and 4-bromobenzo[c][1,2,5]oxadiazole using a procedure essentially the same as for I-47. ¹H NMR (400 MHz, DMSO-d6) δ 8.03 (dd, J=9.1, 0.8 Hz, 1H), 7.68 (dd, J=9.0, 6.7 Hz, 1H), 7.54 (dd, J=6.7, 0.8 Hz, 1H), 7.33 (d, J=11.9 Hz, 1H), 6.97 (d, J=8.3 Hz, 1H), 5.82 (s, 2H).

### Intermediate 70 (I-70)

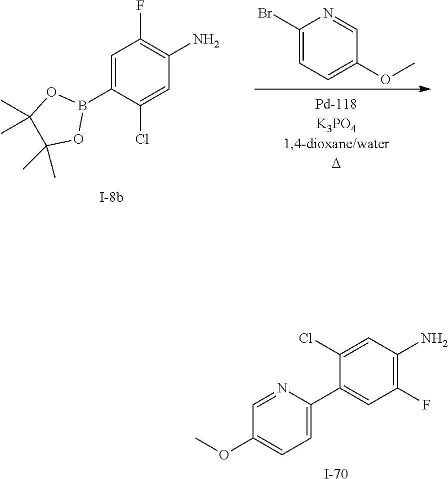

5-Chloro-2-fluoro-4-(5-methoxypyridin-2-yl)aniline I-70 was synthesised from 5-chloro-2-fluoro-4-(4,4,5,5-tetramethyl-1,3,2-dioxaborolan-2-yl)aniline I-8b and 2-bromo-5-methoxypyridine using a procedure essentially the same as for I-47. ¹H NMR (400 MHz, DMSO-d6) δ 8.34 (dd, J=4.3, 2.8 Hz, 1H), 7.66-7.55 (m, 1H), 7.44 (dd, J=8.7, 3.0 Hz, 1H), 7.23 (d, J=12.2 Hz, 1H), 6.88 (d, J=8.3 Hz, 1H), 5.63 (s, 2H), 3.87 (s, 3H).

### Intermediate 71 (I-71)

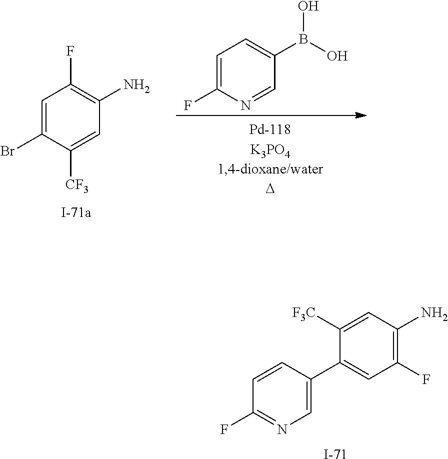

2-fluoro-4-(6-fluoropyridin-3-yl)-5-(trifluoromethyl)aniline I-71 was synthesised from 4-bromo-2-fluoro-5-(trifluoromethyl)aniline I-71a and (6-fluoropyridin-3-yl)boronic acid using a procedure essentially the same as for I-32. ¹H NMR (400 MHz, DMSO-d6) δ 8.13 (d, J=2.5 Hz, 1H), 7.90 (td, J=8.3, 2.6 Hz, 1H), 7.23 (dd, J=8.5, 2.8 Hz, 2H), 7.16 (d, J=11.6 Hz, 1H), 5.82 (s, 2H).

### Intermediate 74 (I-74)

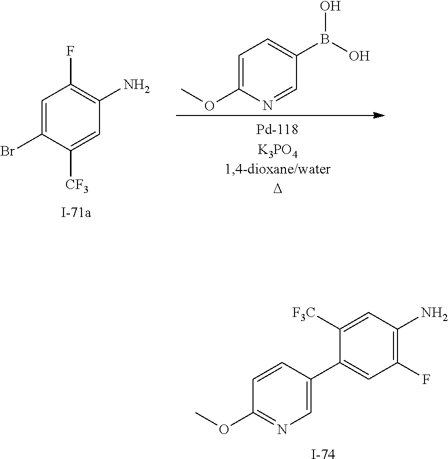

2-Fluoro-4-(6-methoxypyridin-3-yl)-5-(trifluoromethyl)aniline I-74 was synthesised from 4-bromo-2-fluoro-5-(trifluoromethyl)aniline I-71a and (6-methoxypyridin-3-yl)boronic acid using a procedure essentially the same as for I-32. ¹H NMR (400 MHz, DMSO-d6) δ 8.04 (d, J=2.5 Hz, 1H), 7.60 (dd, J=8.4, 2.5 Hz, 1H), 7.22 (d, J=8.7 Hz, 1H), 7.08 (d, J=11.8 Hz, 1H), 6.84 (dd, J=8.4, 0.8 Hz, 1H), 5.74 (s, 2H), 3.88 (s, 3H).

### Intermediate 75 (I-75)

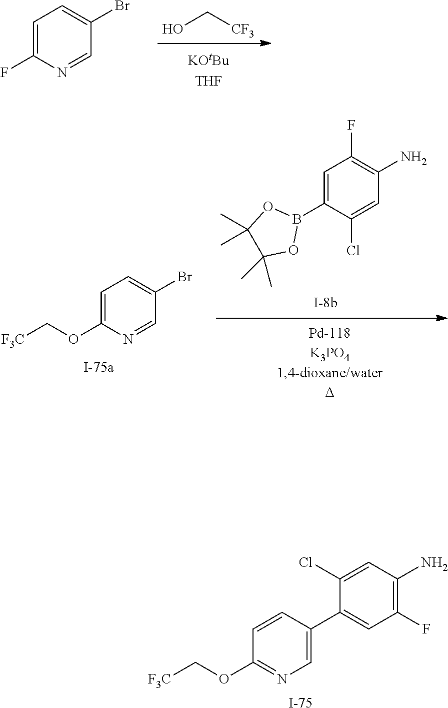

Step 1: To a solution of 5-bromo-2-fluoropyridine (117 μl, 1.14 mmol) and trifluoroethanol (330 μl, 4.55 mmol) in THF (2.5 ml) was added a solution of KO^{t}Bu in THF (2.75 ml, 20% w/w, 4.55 mmol). The reaction was stirred at RT for 24 h before being partitioned between DCM (25 ml) and water (25 ml). The aqueous phase was extracted with additional DCM (25 ml). The organics were combined and washed with water (2×50 ml) and brine (50 ml) and dried over magnesium sulphate. The filtrate was concentrated in vacuo to give 5-bromo-2-(2,2,2-trifluoroethoxy)pyridine I-75a as a colourless oil. ¹H NMR (400 MHz, DMSO-d6) δ 8.35 (dd, J=2.5, 0.7 Hz, 1H), 8.02 (dd, J=8.8, 2.6 Hz, 1H), 7.01 (dd, J=8.8, 0.7 Hz, 1H), 4.98 (q, J=9.0 Hz, 2H).

Step 2: 5-Chloro-2-fluoro-4-(6-(2,2,2-trifluoroethoxy)pyridin-3-yl)aniline I-75 was synthesised from 5-chloro-2-fluoro-4-(4,4,5,5-tetramethyl-1,3,2-dioxaborolan-2-yl)aniline I-8b and 5-bromo-2-(2,2,2-trifluoroethoxy)pyridine I-75a using a procedure essentially the same as for I-47. LCMS (Method 2) m/z 321.3, 323.3 (M+H)⁺ (ES⁺), at 2.15 min

### Intermediate 75 (I-75) Alternative Synthesis

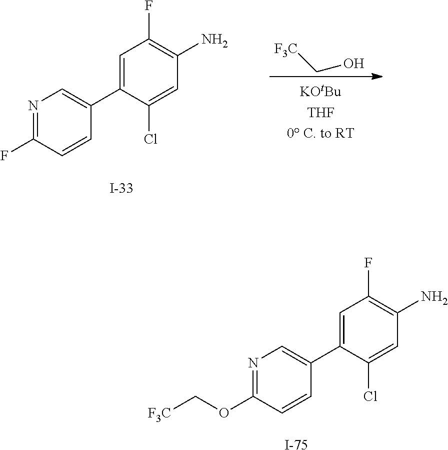

To a solution of 5-chloro-2-fluoro-4-(6-fluoropyridin-3-yl)aniline I-33 (15.0 g, 59.2 mmol) and trifluoroethanol (17.2 ml, 237 mmol) in THF (100 ml) at 0° C. was added a solution of KO^{t}Bu in THF (143 ml, 20% w/w, 237 mmol). The reaction was allowed to warm to RT for 24 h. Water (200 ml) was added and the layers were separated. The organic was washed with more water (200 ml) and separated. The aqueous phase was extracted with additional DCM (3×200 ml). The combined organics were dried over Na₂SO₄ and concentrated in vacuo. The product was purified by chromatography on silica gel (0-30% EtOAc/isohexane) to afford 5-chloro-2-fluoro-4-(6-(2,2,2-trifluoroethoxy)pyridin-3-yl)aniline I-75 as an orange oil. ¹H NMR (400 MHz, DMSO-d6) δ 8.19 (dd, J=2.5, 0.8 Hz, 1H), 7.83 (dd, J=8.6, 2.5 Hz, 1H), 7.15 (d, J=11.9 Hz, 1H), 7.03 (dd, J=8.5, 0.8 Hz, 1H), 6.92 (d, J=8.3 Hz, 1H), 5.63 (s, 2H), 5.02 (q, J=9.1 Hz, 2H),

### Intermediate 76 (I-76)

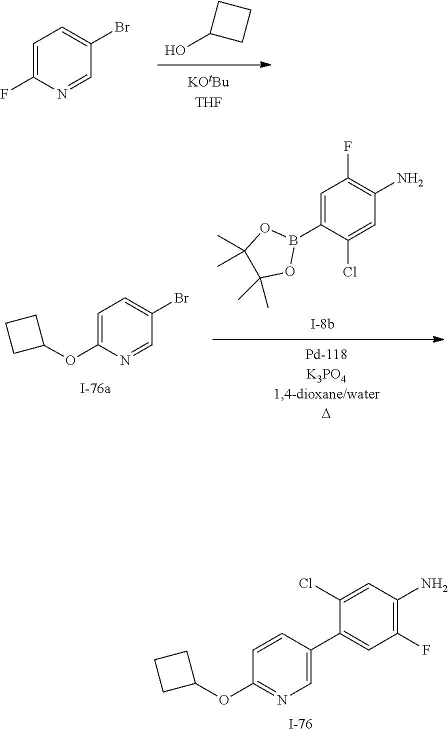

Step 1: 5-Bromo-2-cyclobutoxypyridine I-76a was synthesised from 5-bromo-2-fluoropyridine and cyclobutanol using a procedure essentially the same as for I-75a. LCMS (Method 2) m/z 228.2, 230.3 (M+H)⁺ (ES⁺), at 2.14 min

Step 2: 5-Chloro-4-(6-cyclobutoxypyridin-3-yl)-2-fluoroaniline I-76 was synthesised from 5-chloro-2-fluoro-4-(4,4,5,5-tetramethyl-1,3,2-dioxaborolan-2-yl)aniline I-8b and 5-bromo-2-cyclobutoxypyridine I-76a using a procedure essentially the same as for I-47. LCMS (Method 2) m/z 293.3, 295.3 (M+H)⁺ (ES⁺), at 2.16 min

### Intermediate 77 (I-77)

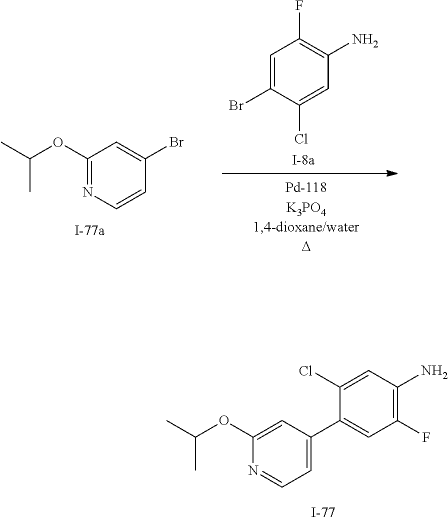

5-Chloro-2-fluoro-4-(2-isopropoxypyridin-4-yl)aniline I-77 was synthesised from 4-bromo-5-chloro-2-fluoroaniline I-8a and 2-(iso-propoxy)pyridine-4-boronic acid I-77a using a procedure essentially the same as for I-32. LCMS (Method 2) m/z 281.7, 283.7 (M+H)⁺ (ES⁺), at 1.95 min

### Intermediate 79 (I-79)

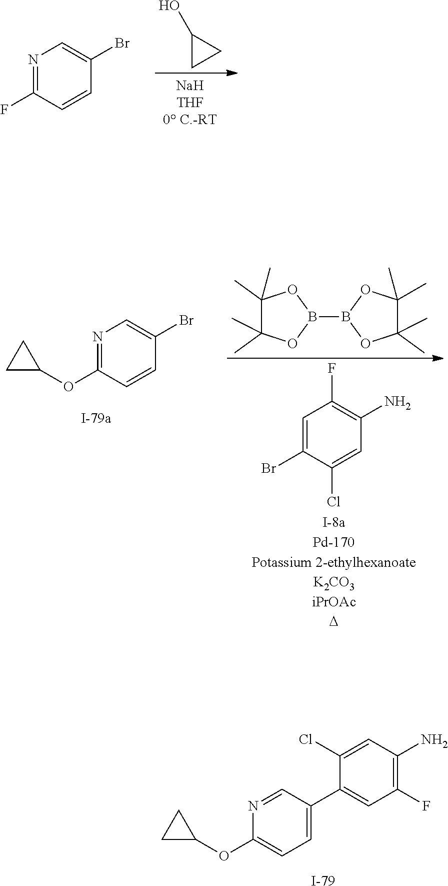

Step 1: To a mixture of sodium hydride (145 mg, 60% w/w, 3.64 mmol) in THF (10 ml) and the resulting suspension cooled to 0° C. To this was added cyclopropanol (0.216 ml, 3.41 mmol) and the resulting suspension stirred at 0° C. for 30 min before 5-bromo-2-fluoropyridine (234 μl, 2.27 mmol) was added. The resultant mixture was warmed to RT for 6 h. Water (10 ml) was added. The reaction mixture was diluted with a 1:1 mixture of aqueous NaCl:NaHCO₃ (25 ml). The product was extracted with DCM (2×25 ml) and the combined organics were concentrated in vacuo. The product was purified by chromatography on silica gel (0-50% EtOAc/isohexane) to afford 5-bromo-2-cyclopropoxypyridine I-79a as a colourless oil. ¹H NMR (400 MHz, DMSO-d6) δ 8.32 (dd, J=2.6, 0.7 Hz, 1H), 7.92 (dd, J=8.8, 2.6 Hz, 1H), 6.87 (dd, J=8.8, 0.7 Hz, 1H), 4.16 (tt, J=6.3, 3.1 Hz, 1H), 0.80-0.73 (m, 2H), 0.66 (dddd, J=6.2, 4.6, 3.1, 0.8 Hz, 2H).

Step 2: 5-Bromo-2-cyclopropoxypyridine I-79a (186 mg, 869 μmol) and 4,4,4′,4′,5,5,5′,5′-octamethyl-2,2′-bi(1,3,2-dioxaborolane) (287 mg, 1.13 mmol) were dissolved in a solution of potassium 2-ethylhexanoate in iPrOAc (2.00 ml, 0.5 M, 1.0 mmol). The resulting solution was sparged with N₂ before Pd-170 (29.3 mg, 43.4 μmol) was added. The reaction mixture heated to 50° C. for 50 min. The reaction mixture was allowed to cool to RT for 30 min before 4-bromo-5-chloro-2-fluoroaniline (195 mg, 869 μmol) and a degassed aqueous solution of K₂CO₃ (1.16 ml, 1.5 M, 1.74 mmol) were added. The reaction mixture was heated to 50° C. for 3.5 h. The reaction mixture was partitioned between brine (25 ml) and DCM (25 ml). The aqueous was extracted with DCM (25 ml) and the combined organics were concentrated in vacuo. The product was purified by chromatography on silica gel (0-15% (0.7 M Ammonia/MeOH)/DCM) to afford 5-chloro-4-(6-cyclopropoxypyridin-3-yl)-2-fluoroaniline I-79 as a pale yellow oil. ¹H NMR (400 MHz, DMSO-d6) δ 8.18 (dd, J=2.5, 0.7 Hz, 1H), 7.75 (dd, J=8.5, 2.5 Hz, 1H), 7.13 (d, J=11.9 Hz, 1H), 6.96-6.85 (m, 2H), 5.60 (s, 2H), 4.22 (tt, J=6.3, 3.1 Hz, 1H), 0.81-0.71 (m, 2H), 0.74-0.63 (m, 2H).

### Intermediate 80 (I-80)

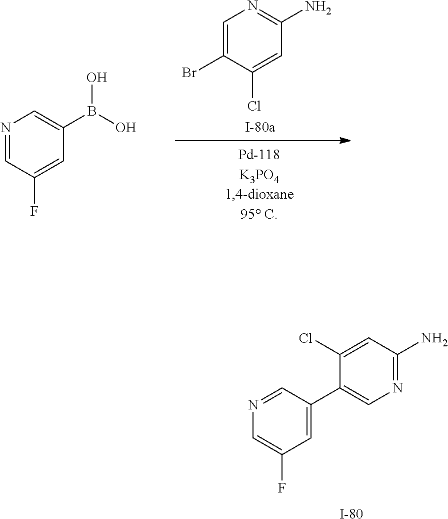

To a solution of (5-fluoropyridin-3-yl)boronic acid (170 mg, 1.21 mmol), 5-bromo-4-chloropyridin-2-amine I-80a (250 mg, 1.21 mmol) and Pd-118 (23.6 mg, 36.2 μmol) in pre-degassed 1,4-dioxane (8.00 ml) was added a pre-degassed aqueous solution of potassium phosphate tribasic (1.81 ml, 2.0 M, 3.62 mmol). The resulting mixture was heated to 95° C. for 8 h. The reaction was cooled to RT and saturated aqueous NaHCO₃ (20 ml) was added and the product was extracted with DCM (3×10 ml). The combined organics were passed through a hydrophobic frit and the filtrate was concentrated in vacuo. The product was purified by chromatography on silica gel (0-80% EtOAc/DCM) to afford 4-chloro-5′-fluoro-[3,3′-bipyridin]-6-amine as a light brown solid. LCMS (Method 2) m/z 224.2, 226.2 (M+H)⁺ (ES⁺), at 0.91 min.

### Intermediate 81 (I-81)

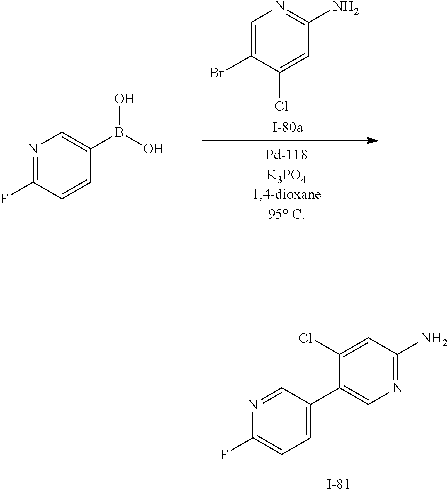

4-Chloro-6′-fluoro-[3,3′-bipyridin]-6-amine I-81 was synthesised from 5-bromo-4-chloropyridin-2-amine I-80a and (6-fluoropyridin-3-yl)boronic acid using a procedure essentially the same as for I-80. LCMS (Method 2) m/z 224.2, 226.2 (M+H)⁺ (ES⁺), at 0.90 min

### Intermediate 82 (I-82)

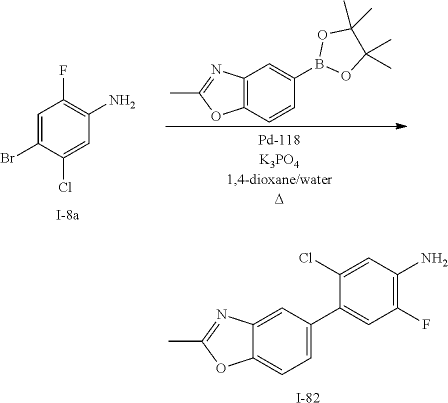

5-chloro-2-fluoro-4-(2-methylbenzo[d]oxazol-5-yl)aniline I-82 was synthesised from 4-bromo-5-chloro-2-fluoroaniline I-8a and 2-methyl-5-(4,4,5,5-tetramethyl-1,3,2-dioxaborolan-2-yl)benzo[d]oxazole using a procedure essentially the same as for I-32. ¹H NMR (400 MHz, DMSO-d6) δ 7.72-7.58 (m, 2H), 7.32 (dd, J=8.4, 1.8 Hz, 1H), 7.11 (d, J=11.9 Hz, 1H), 6.91 (d, J=8.4 Hz, 1H), 5.55 (s, 2H), 2.62 (s, 3H)

### Intermediate 83 (I-83)

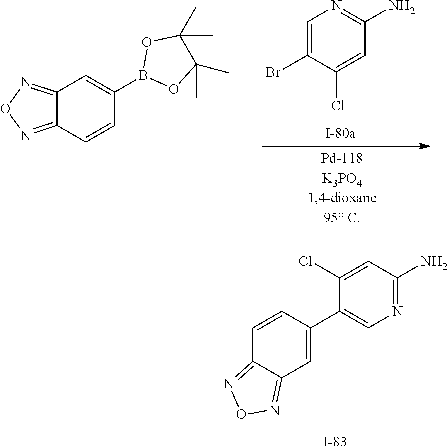

5-(Benzo[c][1,2,5]oxadiazol-5-yl)-4-chloropyridin-2-amine I-83 was synthesised from 5-bromo-4-chloropyridin-2-amine I-80a and 5-(4,4,5,5-tetramethyl-1,3,2-dioxaborolan-2-yl)benzo[c][1,2,5]oxadiazole using a procedure essentially the same as for I-80. ¹H NMR (400 MHz, DMSO-d6) δ 8.11-8.06 (m, 2H), 8.04 (t, J=1.2 Hz, 1H), 7.68 (dd, J=9.3, 1.4 Hz, 1H), 6.65 (s, 1H), 6.60 (s, 2H).

### Intermediate 84 (I-84)

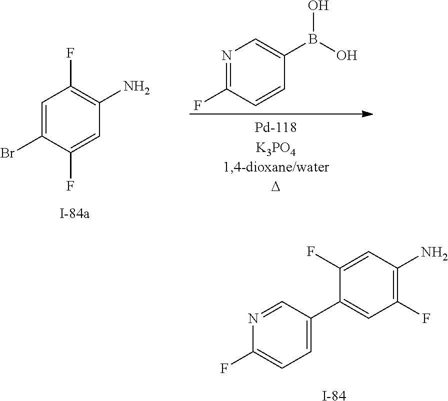

2,5-Difluoro-4-(6-fluoropyridin-3-yl)aniline I-84 was synthesised from 4-bromo-2,5-difluoro-phenylamine I-84a and (6-fluoropyridin-3-yl)boronic acid using a procedure essentially the same as for I-32. ¹H NMR (400 MHz, DMSO-d6) δ 8.35-8.30 (m, 1H), 8.12-8.03 (m, 1H), 7.36-7.20 (m, 2H), 6.65 (dd, J=12.6, 7.6 Hz, 1H), 5.73 (s, 2H).

### Intermediate 85 (I-85)

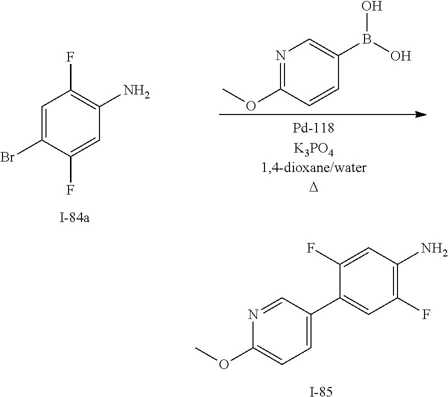

2,5-Difluoro-4-(6-methoxypyridin-3-yl)aniline I-85 was synthesised from 4-bromo-2,5-difluoro-phenylamine I-84a and (6-methoxypyridin-3-yl)boronic acid using a procedure essentially the same as for I-32. ¹H NMR (400 MHz, DMSO-d6) δ 8.28-8.23 (m, 1H), 7.80 (ddd, J=8.6, 2.6, 1.5 Hz, 1H), 7.22 (dd, J=12.0, 7.3 Hz, 1H), 6.86 (dd, J=8.6, 0.8 Hz, 1H), 6.62 (dd, J=12.6, 7.7 Hz, 1H), 5.60 (s, 2H), 3.87 (s, 3H)

### Intermediate 87 (I-87)

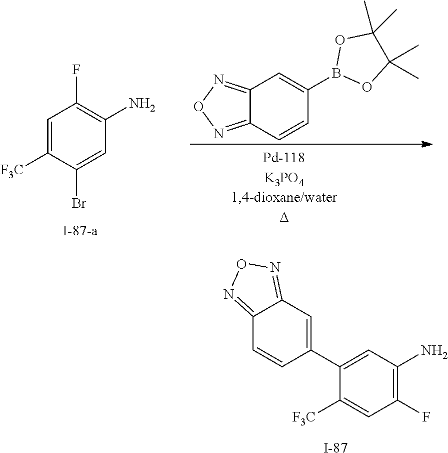

5-(Benzo[c][1,2,5]oxadiazol-5-yl)-2-fluoro-4-(trifluoromethyl)aniline I-87 was synthesised from 5-bromo-2-fluoro-4-(trifluoromethyl)aniline I-87a and 5-(4,4,5,5-tetramethyl-1,3,2-dioxaborolan-2-yl)benzo[c][1,2,5]oxadiazole using a procedure essentially the same as for I-32. ¹H NMR (400 MHz, DMSO-d6) δ 8.10 (dd, J=9.3, 1.1 Hz, 1H), 7.99 (d, J=1.3 Hz, 1H), 7.57-7.45 (m, 2H), 6.77 (d, J=8.6 Hz, 1H), 6.16 (s, 2H).

### Intermediate 88 (I-88)

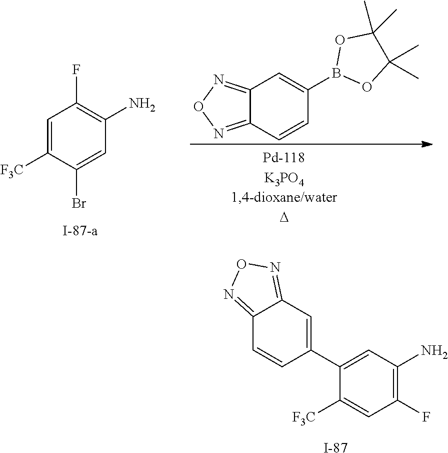

2-Fluoro-5-(5-fluoropyridin-3-yl)-4-(trifluoromethyl)aniline I-88 was synthesised from 5-bromo-2-fluoro-4-(trifluoromethyl)aniline I-87a and (5-fluoropyridin-3-yl)boronic acid using a procedure essentially the same as for I-32. ¹H NMR (400 MHz, DMSO-d6) δ 8.64 (d, J=2.7 Hz, 1H), 8.37 (d, J=1.9 Hz, 1H), 7.75 (dt, J=9.6, 2.3 Hz, 1H), 7.49 (d, J=12.0 Hz, 1H), 6.71 (d, J=8.6 Hz, 1H), 6.13 (s, 2H).

### Intermediate 89 (I-89)

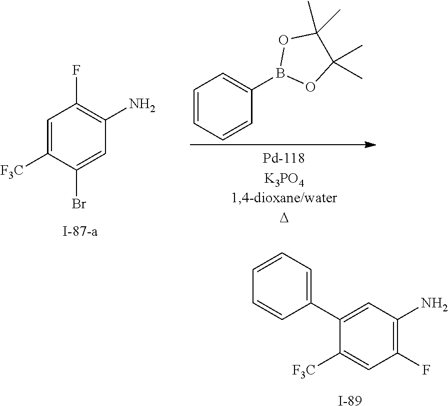

4-Fluoro-6-(trifluoromethyl)-[1,1′-biphenyl]-3-amine I-89 was synthesised from 5-bromo-2-fluoro-4-(trifluoromethyl)aniline I-87a and 4,4,5-trimethyl-2-phenyl-1,3,2-dioxaborolane using a procedure essentially the same as for I-32. ¹H NMR (400 MHz, DMSO-d6) δ 7.46-7.33 (m, 4H), 7.31-7.19 (m, 2H), 6.65 (d, J=8.8 Hz, 1H), 5.99 (s, 2H).

### Intermediate 90 (I-90)

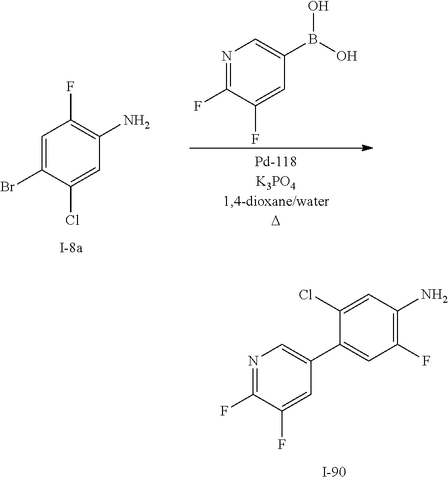

5-Chloro-4-(5,6-difluoropyridin-3-yl)-2-fluoroaniline I-90 was synthesised from 4-bromo-5-chloro-2-fluoroaniline I-8a and (5,6-difluoropyridin-3-yl)boronic acid using a procedure essentially the same as for I-32. ¹H NMR (400 MHz, DMSO-d6) δ 8.14 (ddd, J=10.8, 9.3, 2.1 Hz, 1H), 8.08 (t, J=1.9 Hz, 1H), 7.24 (d, J=11.9 Hz, 1H), 6.93 (d, J=8.3 Hz, 1H), 5.76 (s, 2H).

### Intermediate 91 (I-91)

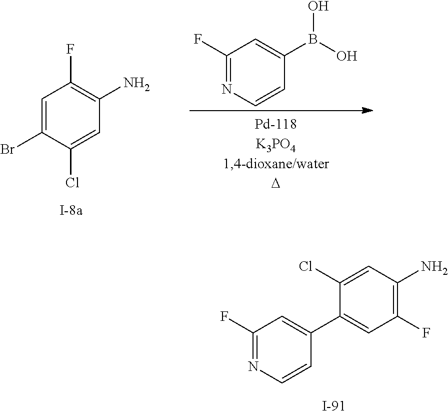

5-Chloro-2-fluoro-4-(2-fluoropyridin-4-yl)aniline I-91 was synthesised from 4-bromo-5-chloro-2-fluoroaniline I-8a and 5-chloro-2-fluoro-4-(2-fluoropyridin-4-yl)aniline using a procedure essentially the same as for I-32. ¹H NMR (400 MHz, DMSO-d6) δ 8.25 (d, J=5.2 Hz, 1H), 7.42 (ddd, J=5.3, 2.1, 1.4 Hz, 1H), 7.27 (d, J=12.0 Hz, 1H), 7.23 (q, J=1.2 Hz, 1H), 6.92 (d, J=8.2 Hz, 1H), 5.87 (s, 2H)

### Intermediate 92 (I-92)

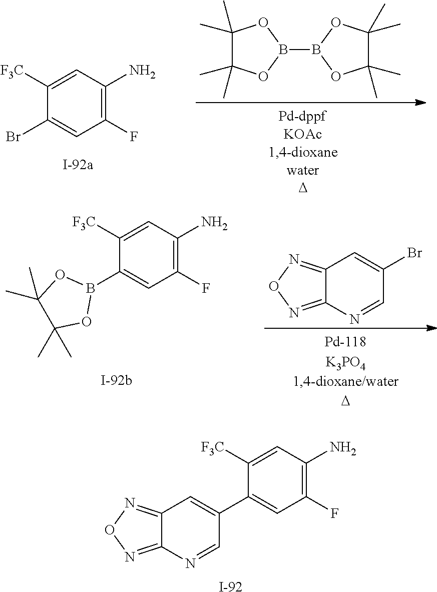

Step 1: 2-Fluoro-4-(4,4,5,5-tetramethyl-1,3,2-dioxaborolan-2-yl)-5-(trifluoromethyl)aniline I-92b was synthesised from 4-bromo-2-fluoro-5-(trifluoromethyl)aniline I-92a using a procedure essentially the same as for I-8b. ¹H NMR (400 MHz, DMSO-d6) δ 7.28 (d, J=11.8 Hz, 1H), 7.14 (dd, J=8.4, 6.5 Hz, 1H), 5.97 (s, 2H), 1.25 (s, 12H).

Step 2: 4-([1,2,5]Oxadiazolo[3,4-b]pyridin-6-yl)-2-fluoro-5-(trifluoromethyl)aniline I-92 was synthesised from 2-fluoro-4-(4,4,5,5-tetramethyl-1,3,2-dioxaborolan-2-yl)-5-(trifluoromethyl)aniline I-92b and 6-bromo-[1,2,5]oxadiazolo[3,4-b]pyridine using a procedure essentially the same as for I-47. ¹H NMR (400 MHz, DMSO-d6) δ 9.01 (dd, J=2.1, 0.9 Hz, 1H), 8.54 (d, J=2.1 Hz, 1H), 7.38 (d, J=11.8 Hz, 1H), 7.29 (d, J=8.5 Hz, 1H), 6.05 (s, 2H)

### Intermediate 93 (I-93)

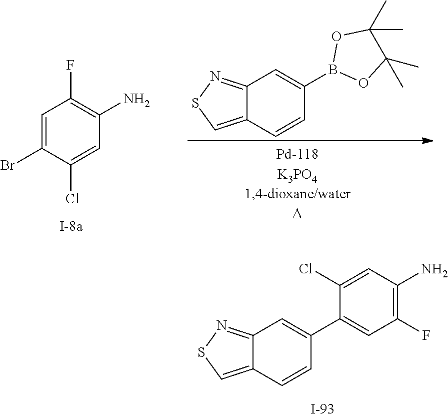

4-(Benzo[c]isothiazol-6-yl)-5-chloro-2-fluoroaniline I-93 was synthesised from 4-bromo-5-chloro-2-fluoroaniline I-8a and 6-(4,4,5,5-tetramethyl-1,3,2-dioxaborolan-2-yl)benzo[c]isothiazole using a procedure essentially the same as for I-32. ¹H NMR (400 MHz, DMSO-d6) δ 9.78 (d, J=1.1 Hz, 1H), 7.95-7.86 (m, 1H), 7.73 (d, J=1.8 Hz, 1H), 7.32 (dd, J=8.9, 1.5 Hz, 1H), 7.22 (d, J=11.9 Hz, 1H), 6.93 (d, J=8.3 Hz, 1H), 5.67 (s, 2H).

### Intermediate 94 (I-94)

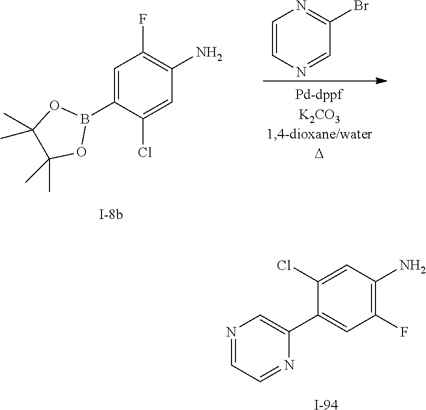

To a stirred solution of 5-chloro-2-fluoro-4-(4,4,5,5-tetramethyl-1,3,2-dioxaborolan-2-yl)aniline I-8b (250 mg, 921 μmol) in 1,4-dioxane (4.00 ml) and water (1.00 ml) was added potassium carbonate (191 mg, 1.38 mmol) and 2-bromopyrazine (100 μl, 1.10 mmol). The reaction mixture was sparged for 5 mins with N₂ prior to the addition of Pd(dppf) (75.2 mg, 92.1 μmol). The reaction mixture was heated at 90° C. for 1 h. The reaction mixture was filtered through celite, the filtrate was diluted in EtOAc. The reaction mixture was diluted with water (25 ml) and the layers were separated. The organics were washed with brine (25 ml), dried over MgSO₄ and concentrated in vacuo. The product was purified by chromatography on silica gel (10-100% EtOAc/DCM) to afford 5-chloro-2-fluoro-4-(pyrazin-2-yl)aniline I-94 as a white solid. ¹H NMR (400 MHz, DMSO-d6) δ 8.90 (d, J=1.6 Hz, 1H), 8.70 (dd, J=2.6, 1.6 Hz, 1H), 8.57 (d, J=2.5 Hz, 1H), 7.36 (d, J=12.0 Hz, 1H), 6.92 (d, J=8.1 Hz, 1H), 5.87 (s, 2H).

### Intermediate 95 (I-95)

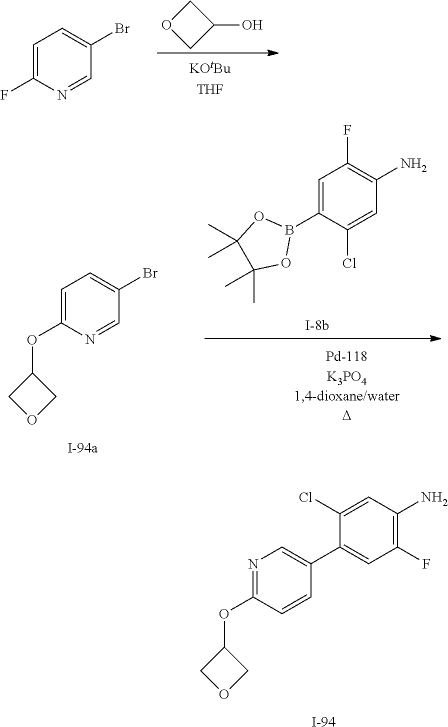

Step 1: To a solution of 5-bromo-2-fluoropyridine (200 mg, 1.14 mmol) and oxetan-3-ol (299 μl, 4.55 mmol) in THF (2.50 ml) was added a solution of KO^{t}Bu in THF (2.75 mL, 20% w/w, 4.55 mmol). The reaction was stirred at RT for 24 h. The reaction mixture was diluted with DCM (25 ml) and water (25 ml) and the layers were separated. The aqueous phase was extracted with DCM (25 ml). The combined organics were washed with water (2×50 ml) and brine (50 ml) and dried over MgSO₄. The organics were concentrated in vacuo to give 5-bromo-2-(2,2,2-trifluoroethoxy)pyridine I-94a as a colourless oil. LCMS (Method 2) m/z 229.7, 231.7 (M+H)⁺ (ES⁺), at 1.56 min.

Step 2: 5-chloro-2-fluoro-4-(6-(oxetan-3-yloxy)pyridine-3-yl)aniline I-94 was synthesised from 5-bromo-2-(2,2,2-trifluoroethoxy)pyridine I-94a and 5-chloro-2-fluoro-4-(4,4,5,5-tetramethyl-1,3,2-dioxaborolan-2-yl)aniline I-8b using a procedure essentially the same as for I-47. LCMS (Method 2) m/z 295.3, 297.3 (M+H)⁺ (ES⁺), at 1.73 min.

### Intermediate 96 (I-96)

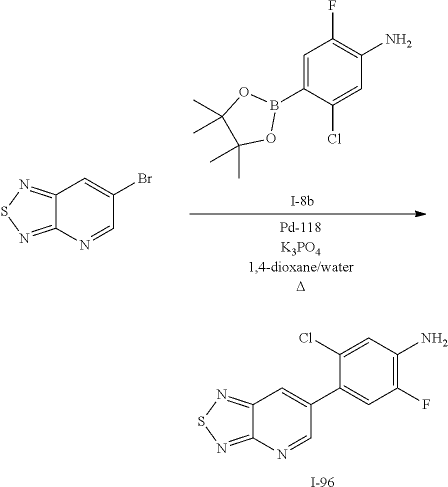

4-([1,2,5]Thiadiazolo[3,4-b]pyridine-6-yl)-5-chloro-2-fluoroaniline I-95 was synthesised from 5-chloro-2-fluoro-4-(4,4,5,5-tetramethyl-1,3,2-dioxaborolan-2-yl)aniline I-8b and 6-bromo-[1,2,5]thiadiazolo[3,4-b]pyridine using a procedure that is essentially the same as for I-47.

¹H NMR (400 MHz, DMSO-d6) δ 9.18 (d, J=2.3 Hz, 1H), 8.54 (d, J=2.3 Hz, 1H), 7.41 (d, J=11.9 Hz, 1H), 6.99 (d, J=8.2 Hz, 1H), 5.84 (s, 2H)

### Intermediate 97 (I-97)

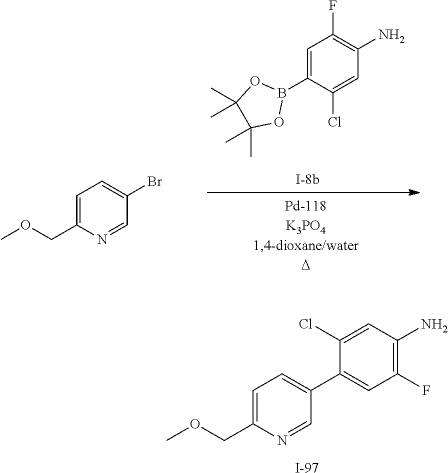

5-Chloro-2-fluoro-4-(6-(methoxymethyl)pyridine-3-yl)aniline I-97 was synthesised from 5-chloro-2-fluoro-4-(4,4,5,5-tetramethyl-1,3,2-dioxaborolan-2-yl)aniline I-8b and 5-bromo-2-(methoxymethyl)pyridine using a procedure essentially the same as for I-47. ¹H NMR (400 MHz, DMSO-d6) δ 8.52 (d, J=2.2 Hz, 1H), 7.83 (dd, J=8.1, 2.3 Hz, 1H), 7.44 (d, J=8.0 Hz, 1H), 7.17 (d, J=11.9 Hz, 1H), 6.93 (d, J=8.3 Hz, 1H), 5.66 (s, 2H), 4.52 (s, 2H), 3.39 (s, 3H).

### Intermediate 98 (I-98)

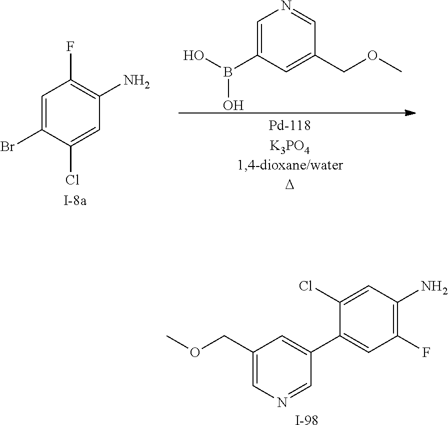

5-Chloro-2-fluoro-4-(5-(methoxymethyl)pyridine-3-yl)aniline I-98 was synthesised from 4-bromo-5-chloro-2-fluoroaniline I-8a and (5-(methoxymethyl)pyridine-3-yl)boronic acid using a procedure essentially the same as for I-32. ¹H NMR (400 MHz, DMSO-d6) δ 8.51 (d, J=2.2 Hz, 1H), 8.48 (d, J=2.0 Hz, 1H), 7.74 (t, J=2.2 Hz, 1H), 7.18 (d, J=11.9 Hz, 1H), 6.93 (d, J=8.3 Hz, 1H), 5.67 (s, 2H), 4.49 (s, 2H), 3.33 (s, 3H).

### Intermediate 99 (I-99)

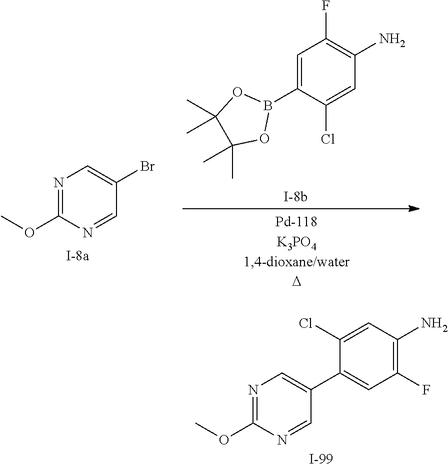

5-Chloro-2-fluoro-4-(2-methoxypyrimidin-5-yl)aniline I-99 was synthesised from 5-chloro-2-fluoro-4-(4,4,5,5-tetramethyl-1,3,2-dioxaborolan-2-yl)aniline I-8b and 5-bromo-2-methoxypyrimidine using a procedure essentially the same as for I-47. ¹H NMR (400 MHz, DMSO-d6) δ 8.63 (s, 2H), 7.23 (d, J=12.0 Hz, 1H), 6.93 (d, J=8.3 Hz, 1H), 5.69 (s, 2H), 3.95 (s, 3H)

### Intermediate 100 (I-100)

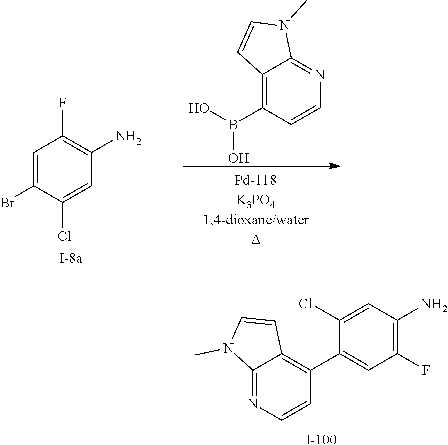

5-Chloro-2-fluoro-4-(1-methyl-1H-pyrrolo[2,3-b]pyridine-4-yl)aniline I-100 was synthesised from 4-bromo-5-chloro-2-fluoroaniline I-8a and 1-methyl-4-(4,4,5,5-tetramethyl-1,3,2-dioxaborolan-2-yl)-1H-pyrrolo[2,3-b]pyridine using a procedure essentially the same as for I-32. ¹H NMR (400 MHz, DMSO-d6) δ 8.28 (d, J=4.9 Hz, 1H), 7.53 (d, J=3.5 Hz, 1H), 7.13 (d, J=11.8 Hz, 1H), 7.03 (d, J=4.9 Hz, 1H), 6.96 (d, J=8.3 Hz, 1H), 6.26 (d, J=3.5 Hz, 1H), 5.69 (s, 2H), 3.84 (s, 3H).

### Intermediate 101 (I-101)

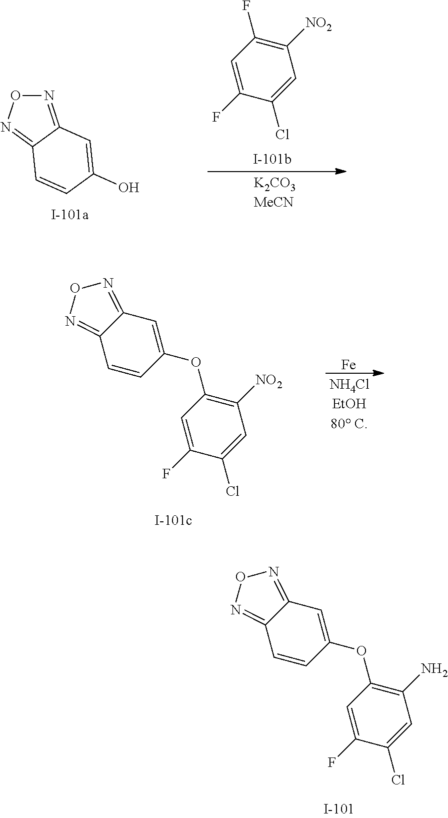

Step 1: To a solution of 1-chloro-2,4-difluoro-5-nitrobenzene I-101a (580 mg, 3.00 mmol) and benzo[c][1,2,5]oxadiazol-5-ol I-101b (410 mg, 3.02 mmol) in MeCN (20 ml) was added K₂CO₃ (1.24 g, 8.98 mmol). The reaction was stirred at RT for 3 h. The reaction mixture was diluted with water (5 ml) before the product was extracted with DCM (3×10 ml). The combined organics were concentrated in vacuo. The product was purified by chromatography on silica gel (3% EtOAc/petroleum ether) to give 5-(4-chloro-5-fluoro-2-nitrophenoxy)benzo[c][1,2,5]oxadiazole I-101c as a yellow solid. ¹H NMR (400 MHz, DMSO-d6) δ 8.60 (d, J=7.6 Hz, 1H), 8.19 (d, J=9.6 Hz, 1H), 7.88 (d, J=10.4 Hz, 1H), 7.59 (dd, J=2.0, 9.6 Hz, 1H), 7.53-7.50 (m, 1H)

Step 2: To a solution of 5-(4-chloro-5-fluoro-2-nitrophenoxy)benzo[c][1,2,5]oxadiazole I-101c (70 mg, 0.23 mmol) in EtOH (2 ml) was added a saturated aqueous NH₄Cl solution (1 ml) and Fe (50 mg, 0.90 mmol). The reaction was stirred at 80° C. for 30 mins. The reaction mixture was cooled and filtered and diluted with water (5 ml). The product was extracted with DCM (3×10 ml). The combined organics were concentrated in vacuo. The product was purified by prep-TLC (25% EtOAc/petroleum ether) to give 2-(benzo[c][1,2,5]oxadiazol-5-yloxy)-5-chloro-4-fluoroaniline I-101 as a yellow solid. LCMS (Method 5) m/z 279.9, 281.9. (M+H)⁺ (ES⁺), at 1.78 min

### Intermediate 102 (I-102)

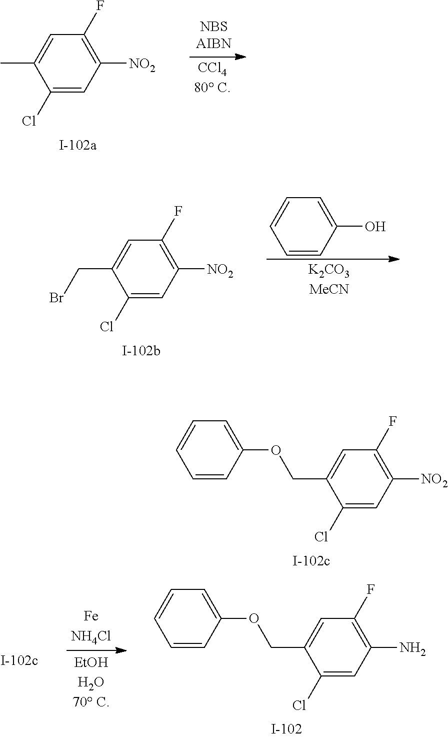 [SMILES: Cc1cc(F)c([N+](=O)[O-])cc1Cl ; O=[N+]([O-])c1cc(Cl)c(CBr)cc1F ; Oc1ccccc1 ; O=[N+]([O-])c1cc(Cl)c(COc2ccccc2)cc1F ; Nc1cc(Cl)c(COc2ccccc2)cc1F]

Step 1: To a solution of 1-chloro-4-fluoro-2-methyl-5-nitrobenzene I-102a (3 g, 15.8 mmol) in CCl₄ (60 ml) was added AlBN (260 mg, 1.58 mmol) and NBS (3.1 g, 17.38 mmol) at RT. The mixture was heated at 80° C. for 16 h. The reaction mixture was concentrated in vacuo. The product was purified by chromatography on silica gel (1% EtOAc/petroleum ether) to give 1-(bromomethyl)-2-chloro-5-fluoro-4-nitrobenzene I-102b as a colourless oil. ¹H NMR (400 MHz, DMSO-d6) δ 8.32 (d, J=6.8 Hz, 1H), 7.95 (d, J=11.6 Hz, 1H), 4.75 (s, 2H).

Step 2: To a solution of 1-(bromomethyl)-2-chloro-5-fluoro-4-nitrobenzene I-102b (2 g, 5.61 mmol) in MeCN (45 ml) was added K₂CO₃ (2.33 g, 16.83 mmol) and phenol (528 mg, 5.61 mmol). After stirring at RT for 2 h, the mixture was diluted with water (10 ml). The product was extracted with DCM (3×30 ml). The combined organics were dried over Na₂SO₄ and concentrated in vacuo. The product was purified by chromatography on silica gel (2% EtOAc/petroleum ether) to give 1-chloro-4-fluoro-5-nitro-2-(phenoxymethyl)benzene I-102c as a light yellow solid. LCMS (Method 1) m/z 282 (M+H)⁺ (ES⁺), at 1.76 min.

Step 3: To a solution of 1-chloro-4-fluoro-5-nitro-2-(phenoxymethyl)benzene I-102c (200 mg, 0.71 mmol) in EtOH (4 ml) was added a solution of NH₄Cl (305 mg, 5.68 mmol) in water (2 ml) and Fe (200 mg, 3.55 mmol) at RT. After stirring at 70° C. for 2 h, the reaction mixture was quenched with water (10 ml) and the product was extracted with DCM (3×30 ml). The combined organics were dried over Na₂SO₄ and concentrated in vacuo. The product was purified by chromatography on silica gel (2.5% EtOAc/petroleum ether) to give 5-chloro-2-fluoro-4-(phenoxymethyl)aniline as a white solid. ¹H NMR (400 MHz, DMSO-d6) δ 7.29-7.27 (m, 3H), 6.99-6.83 (m, 4H), 5.54 (s, 2H), 4.93 (s, 2H).

### Intermediate 103 (I-103)

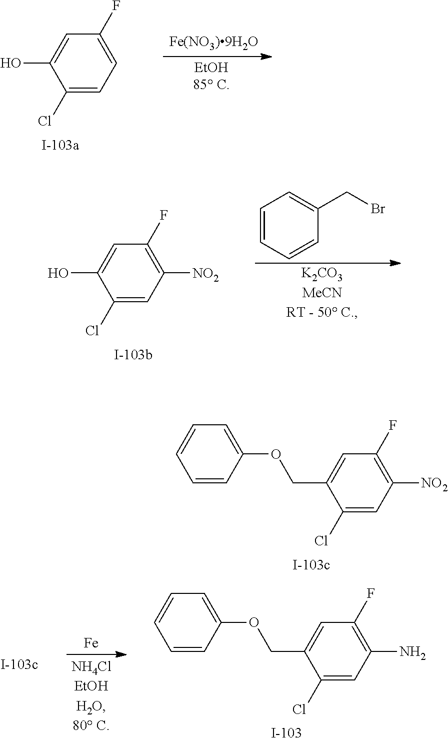

Step 1: To a solution of 2-chloro-5-fluorophenol I-103a (200 mg, 1.37 mmol) in EtOH (5 ml) was added Fe(NO₃)₃·9H₂O (553 mg, 1.37 mmol). The solution was stirred at 85° C. for 4 h. The mixture was filtered and the filtrate was concentrated in vacuo. The product was purified by prep-TLC (50% EtOAc/petroleum ether) to give 2-chloro-5-fluoro-4-nitrophenol I-103b as a yellow solid. ¹H NMR (400 MHz, DMSO-d6) δ 8.20 (d, J=8.0 Hz, 1H), 7.63-7.60 (m, 1H), 6.94-6.90 (m, 1H).

Step 2: To a solution of 2-chloro-5-fluoro-4-nitrophenol I-103b (163 mg, 0.85 mmol) in MeCN (4 ml) was added benzyl bromide (145 mg, 0.85 mmol) and K₂CO₃ (352 mg, 2.55 mmol). The reaction mixture was stirred at 50° C. for 1 h. The reaction mixture was cooled and diluted with water (20 ml) and the product was extracted with DCM (3×20 ml). The combined organics were dried over Na₂SO₄ and concentrated in vacuo. The product was purified by prep-TLC (10% EtOAc/petroleum ether) to give 1-(benzyloxy)-2-chloro-5-fluoro-4-nitrobenzene I-103c as a yellow oil. ¹H NMR (400 MHz, DMSO-d6) δ 8.32 (d, J=8.0 Hz, 1H), 7.58 (d, J=13.2 Hz, 1H), 7.50-7.36 (m, 5H), 5.37 (s, 2H).

Step 3: 4-(Benzyloxy)-5-chloro-2-fluoroaniline I-103 was synthesised from 1-(benzyloxy)-2-chloro-5-fluoro-4-nitrobenzene I-103c using a procedure essentially the same as for I-101. LCMS (Method 5) m/z 252.1 (M+H)⁺ (ES⁺), at 1.68 min

### Intermediate 105 (I-105)

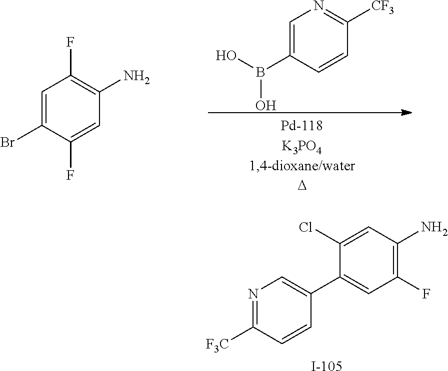

2,5-Difluoro-4-(6-(trifluoromethyl)pyridine-3-yl)aniline I-105 was synthesised from 4-bromo-2,5-difluoroaniline and (6-(trifluoromethyl)pyridine-3-yl)boronic acid using a procedure essentially the same as for I-32. ¹H NMR (400 MHz, CDCl₃) δ 8.83 (s, 1H), 8.01-7.94 (m, 1H), 7.72 (dd, J=8.2, 0.9 Hz, 1H), 7.11 (dd, J=11.1, 6.8 Hz, 1H), 6.61 (dd, J=11.5, 7.4 Hz, 1H), 2 exchangeable protons not observed.

### Intermediate 106 (I-106)

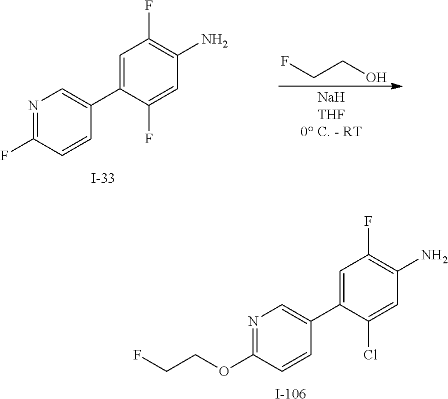

To a suspension of sodium hydride (80 mg, 60% w/w, 1.99 mmol) in THF (8 ml) at 0° C. was added 2-fluoroethan-1-ol (110 μl, 1.87 mmol) and the resulting mixture was stirred at 0° C. for 45 min before 5-chloro-2-fluoro-4-(6-fluoropyridin-3-yl)aniline I-33 (300 mg, 1.25 mmol) was added. The reaction mixture was allowed to warm to RT for 20 h. The reaction mixture was partitioned between brine (25 ml) and DCM (25 ml) and the layers were separated. The aqueous was extracted with DCM (25 ml). The combined organics were concentrated in vacuo. The product was purified by chromatography on silica gel (0-35% EtOAc/isohexane) to afford 5-chloro-2-fluoro-4-(6-(2-fluoroethoxy)pyridin-3-yl)aniline I-106 as a yellow oil. ¹H NMR (400 MHz, DMSO-d6) δ 8.14 (dd, J=2.5, 0.7 Hz, 1H), 7.76 (dd, J=8.6, 2.5 Hz, 1H), 7.13 (d, J=11.9 Hz, 1H), 6.92-6.92 (m, 1H), 6.91-6.89 (m, 1H), 5.61 (s, 2H), 4.85-4.79 (m, 1H), 4.72-4.68 (m, 1H), 4.59-4.54 (m, 1H), 4.52-4.46 (m, 1H).

### Intermediate 107 (I-107)

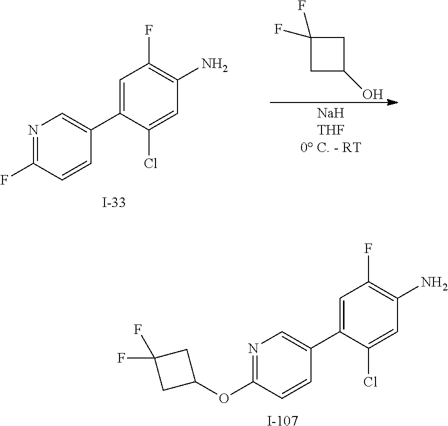

5-Chloro-4-(6-(3,3-difluorocyclobutoxy)pyridin-3-yl)-2-fluoroaniline I-107 was synthesised from 5-chloro-2-fluoro-4-(6-fluoropyridin-3-yl)aniline I-33 and 3,3-difluorocyclobutan-1-ol using a procedure essentially the same as for I-106. ¹H NMR (400 MHz, DMSO-d6) δ 8.15 (dd, J=2.5, 0.7 Hz, 1H), 7.77 (dd, J=8.6, 2.5 Hz, 1H), 7.12 (d, J=11.9 Hz, 1H), 6.94-6.86 (m, 2H), 5.61 (s, 2H), 5.14 (t, J=6.4 Hz, 1H), 3.26-3.08 (m, 2H), 2.83-2.64 (m, 2H).

### Intermediate 108 (I-108)

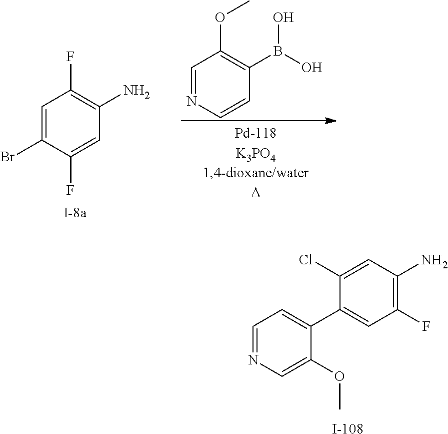

5-Chloro-2-fluoro-4-(3-methoxypyridin-4-yl)aniline I-108 was synthesised from 4-bromo-5-chloro-2-fluoroaniline I-8a and (3-methoxypyridin-4-yl)boronic acid using a procedure essentially the same as for I-32. ¹H NMR (400 MHz, CDCl₃) δ 8.36 (s, 1H), 8.30 (d, J=4.9 Hz, 1H), 7.20 (d, J=4.8 Hz, 1H), 6.94 (d, J=11.1 Hz, 1H), 6.88 (d, J=8.1 Hz, 1H), 3.90 (s, 3H). 2 exchangeable protons not observed.

### Intermediate 110 (I-110)

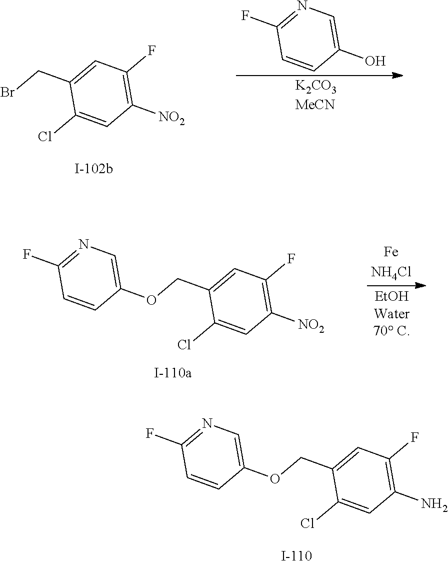 [SMILES: O=[N+]([O-])c1cc(Cl)c(CBr)cc1F ; Oc1ccc(F)nc1 ; O=[N+]([O-])c1cc(Cl)c(COc2ccc(F)nc2)cc1F ; Nc1cc(Cl)c(COc2ccc(F)nc2)cc1F]

Step 1: To a solution of 1-(bromomethyl)-2-chloro-5-fluoro-4-nitrobenzene I-102b (236 mg, 0.88 mmol) in MeCN (3 ml) were added K₂CO₃ (367 mg, 2.65 mmol) and 6-fluoropyridin-3-ol (100 mg, 0.88 mmol). After stirring at RT for 2 h, the reaction was diluted with water (10 ml). The product was extracted with DCM (3×30 ml), the combined organics were dried over Na₂SO₄ and concentrated in vacuo. The product was purified by prep-TLC (20% EtOAc/petroleum ether) to give 5-((2-chloro-5-fluoro-4-nitrobenzyl)oxy)-2-fluoropyridine I-110-a as a light yellow solid. LCMS (Method 5) m/z 301.0 (M+H)⁺ (ES⁺), at 1.52 min.

Step 2: To a solution of 5-((2-chloro-5-fluoro-4-nitrobenzyl)oxy)-2-fluoropyridine I-110-a (100 mg, 0.33 mmol) in EtOH (4 ml) was added Fe (94 mg, 1.66 mmol) and a solution of NH₄Cl (144 mg, 2.66 mmol) in water (2 ml) at RT. After stirring at 70° C. for 2 h, the reaction was diluted with water (10 ml). The product was extracted with DCM (3×30 ml). The combined organics were dried over Na₂SO₄ and concentrated in vacuo. The product was purified by chromatography on silica gel (20% EtOAc/petroleum ether) to give 5-chloro-2-fluoro-4-(((6-fluoropyridin-3-yl)oxy)methyl)aniline I-110 as a white solid. ¹H NMR (400 MHz, DMSO-d6) δ 7.95 (s, 1H), 7.68-7.64 (m, 1H), 7.26 (d, J=7.6 Hz, 1H), 7.12 (dd, J=9.2, 3.6 Hz, 1H), 6.84 (d, J=8.0 Hz, 1H), 5.60 (s, 2H), 5.00 (s, 2H).

### Intermediate 111 (I-111)

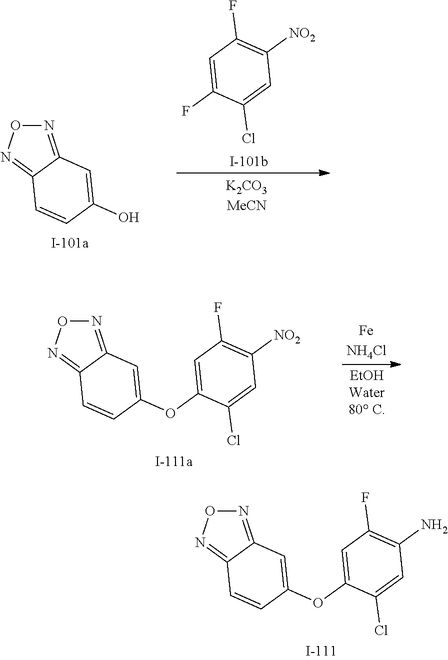 [SMILES: Oc1ccc2nonc2c1 ; O=[N+]([O-])c1cc(Cl)c(F)cc1F ; O=[N+]([O-])c1cc(Cl)c(Oc2ccc3nonc3c2)cc1F ; Nc1cc(Cl)c(Oc2ccc3nonc3c2)cc1F]

Step 1: To a solution of 1-chloro-2,4-difluoro-5-nitrobenzene I-101b (159 mg, 0.82 mmol) and benzo[c][1,2,5]oxadiazol-5-ol I-101a (112 mg, 0.82 mmol) in MeCN (10 ml) was added K₂CO₃ (341 mg, 2.47 mmol). The reaction was stirred at RT for 3 h. The reaction mixture was diluted with water (5 ml) and the product was extracted with DCM (3×10 ml). The combined organics were concentrated in vacuo and the product was purified by chromatography on silica gel (3% EtOAc/petroleum ether) to give 5-(2-chloro-5-fluoro-4-nitrophenoxy)benzo[c][1,2,5]oxadiazole I-111a as a white solid. ¹H NMR (400 MHz, DMSO-d6) δ 8.56 (d, J=7.6 Hz, 1H), 8.24-8.21 (m, 1H), 7.76 (dd, J=9.6, 2.0 Hz, 1H), 7.63-7.59 (m, 2H).

Step 2: To a solution of 5-(2-chloro-5-fluoro-4-nitrophenoxy)benzo[c][1,2,5]oxadiazole I-111a (130 mg 0.42 mmol) in EtOH (4 ml) was added a saturated aqueous solution of NH₄Cl (2 ml) and Fe (94 mg, 1.68 mmol). The reaction was stirred at 80° C. for 30 min. The reaction mixture was filtered and diluted with water (5 ml) before the product was extracted with DCM (3×10 ml). The combined organics were concentrated in vacuo and the product was purified by prep-TLC (25% EtOAc/petroleum ether) to give 4-(benzo[c][1,2,5]oxadiazol-5-yloxy)-5-chloro-2-fluoroaniline I-111 as a yellow solid. ¹H NMR (400 MHz, DMSO-d6) δ 8.12 (d, J=9.6 Hz, 1H), 7.53 (dd, J=9.6, 2.0 Hz, 1H), 7.28 (d, J=11.6 Hz, 1H), 6.97 (d, J=8.8 Hz, 1H), 6.84 (t, J=0.8 Hz, 1H), 5.54 (s, 2H).

### Intermediate 113 (I-113)

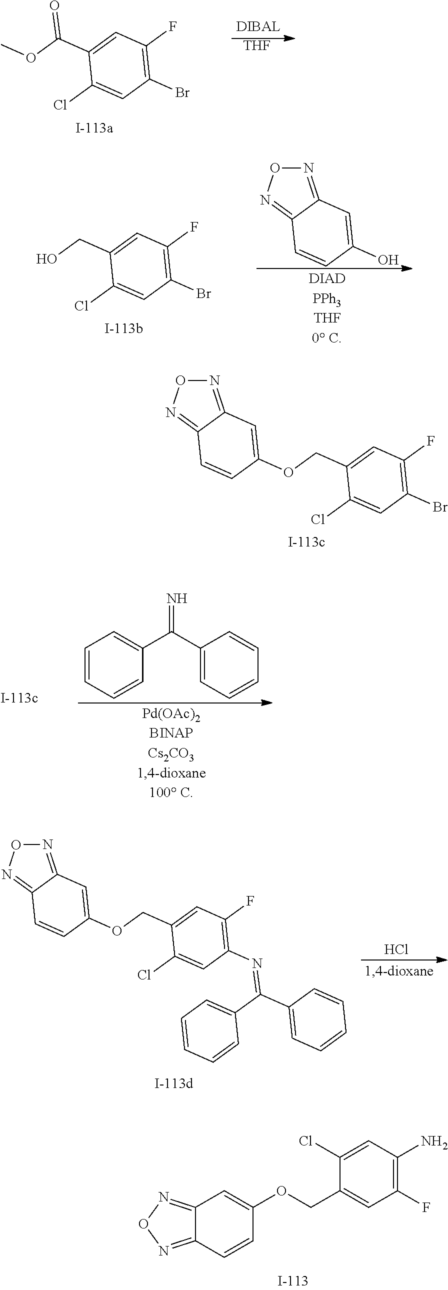

Step 1: To a solution of methyl 4-bromo-2-chloro-5-fluorobenzoate I-113a (400 mg, 1.49 mmol) in THF (3 ml) was added a solution of DIBAL in hexanes (3.8 ml, 1 M, 3.80 mmol) at RT. The resultant reaction mixture was stirred at RT for 2 h. Water (15 ml) was added and the product was extracted with EtOAc (3×15 ml). The combined organics were washed with brine (15 ml), dried over Na₂SO₄ and concentrated in vacuo to give (4-bromo-2-chloro-5-fluorophenyl)methanol I-113b as a white solid. ¹H NMR (400 MHz, DMSO-d6): 5 ppm: 7.84 (d, J=6.0 Hz, 1H), 7.44 (d, J=9.6 Hz, 1H), 5.63 (t, J=5.2 Hz, 1H), 4.50 (d, J=4.8 Hz, 2H).

Step 2: To a solution of benzo[c][1,2,5]oxadiazol-5-ol (227 mg, 1.67 mmol), (4-bromo-2-chloro-5-fluorophenyl)methanol I-113b (400 mg, 1.67 mmol) and triphenylphosphine (879 mg, 3.34 mmol) in THF (20 ml) at 0° C. was added DIAD (674 mg, 3.34 mmol). The reaction was stirred at RT for 16 h. Water (40 ml) was added and the product was extracted with EtOAc (3×40 ml). The combined organics were washed with brine (40 ml), dried over Na₂SO₄ and concentrated in vacuo. The product was purified by chromatography on silica gel (20% EtOAc/petroleum ether) to give 5-((4-bromo-2-chloro-5-fluorobenzyl)oxy)benzo[c][1,2,5]oxadiazole 113-c as a white solid. ¹H NMR (400 MHz, DMSO-d6) δ 8.00-7.97 (m, 2H), 7.75 (d, J=9.2 Hz, 1H), 7.41-7.38 (m, 2H), 5.29 (s, 2H).

Step 3: To a solution of 5-((4-bromo-2-chloro-5-fluorobenzyl)oxy)benzo[c][1,2,5]oxadiazole 113c (300 mg, 0.84 mmol), diphenylmethanimine (152 mg, 0.84 mmol), Pd(OAc)₂ (19 mg, 0.084 mmol) and BINAP (53 mg, 0.084 mmol) in 1,4-dioxane (10 ml) was added Cs₂CO₃ (819 mg, 2.52 mmol). The reaction was stirred at 100° C. for 16 h. The reaction was cooled to RT. Water (20 ml) was added and the product was extracted with EtOAc (3×20 ml). The combined organics were washed with brine (20 m), dried over Na₂SO₄ and concentrated in vacuo. The product was purified by chromatography on silica gel (20% EtOAc/petroleum ether) to give N-(4-((benzo[c][1,2,5]oxadiazol-5-yloxy)methyl)-5-chloro-2-fluorophenyl)-1,1-diphenylmethanimine 113-d as a white solid. ¹H NMR (400 MHz, DMSO-d6) δ 7.98 (d, J=9.6 Hz, 1H), 7.70-7.67 (m, 2H), 7.61-7.57 (m, 1H), 7.52-7.46 (m, 3H), 7.41-7.34 (m, 5H), 7.23-7.20 (m, 2H), 7.10 (d, J=7.2 Hz, 1H), 5.14 (s, 2H).

Step 4: To a solution of N-(4-((benzo[c][1,2,5]oxadiazol-5-yloxy)methyl)-5-chloro-2-fluorophenyl)-1,1-diphenylmethanimine 113-d (150 mg, 0.33 mmol) in 1,4-dioxane (2 ml) was added 4 M HCl in 1,4-dioxane (2 ml, 8 mmol). The reaction was stirred at RT for 2 h. The reaction mixture was basified to pH=8 by the addition of saturated aqueous NaHCO₃ and the product was extracted with EtOAc (3×10 ml). The combined organics were washed with brine (20 ml), dried over Na₂SO₄ and concentrated in vacuo to give 4-((benzo[c][1,2,5]oxadiazol-5-yloxy)methyl)-5-chloro-2-fluoroaniline I-113 as a yellow solid. LCMS (Method 5) m/z 294.0 (M+H)⁺ (ES⁺), at 1.45 min.

### Intermediate 114 (I-114)

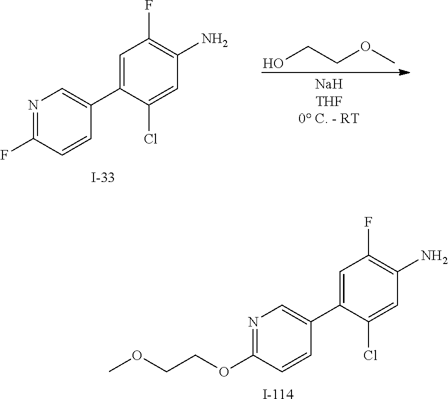

5-Chloro-2-fluoro-4-(6-(2-methoxyethoxy)pyridin-3-yl)aniline I-114 was synthesised from 5-chloro-2-fluoro-4-(6-fluoropyridin-3-yl)aniline I-33 and 2-methoxyethan-1-ol using a procedure essentially the same as for I-106. ¹H NMR (400 MHz, DMSO-d6) δ 8.13 (dd, J=2.6, 0.9 Hz, 1H), 7.73 (dd, J=8.6, 2.5 Hz, 1H), 7.12 (d, J=12.0 Hz, 1H), 6.90 (d, J=8.4 Hz, 1H), 6.86 (dd, J=8.6, 0.8 Hz, 1H), 5.60 (s, 2H), 4.49-4.30 (m, 2H), 3.73-3.59 (m, 2H), 3.30 (s, 3H).

### Intermediate 115 (I-115)

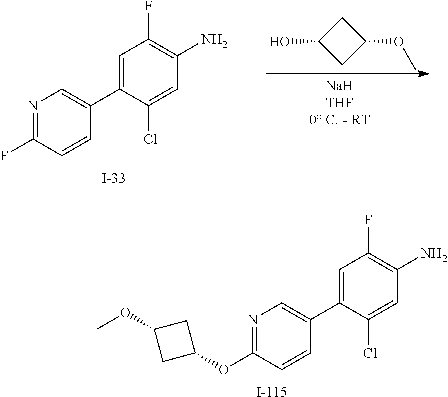

5-Chloro-2-fluoro-4-(6-((1s,3s)-3-methoxycyclobutoxy)pyridin-3-yl)aniline I-115 was synthesised from 5-chloro-2-fluoro-4-(6-fluoropyridin-3-yl)aniline I-33 and (1s,3s)-3-methoxycyclobutan-1-ol using a procedure essentially the same as for I-106. ¹H NMR (400 MHz, DMSO-d6) δ 8.11 (dd, J=2.6, 0.9 Hz, 1H), 7.72 (dd, J=8.6, 2.6 Hz, 1H), 7.12 (d, J=11.9 Hz, 1H), 6.90 (d, J=8.3 Hz, 1H), 6.83 (dd, J=8.6, 0.9 Hz, 1H), 5.59 (s, 2H), 4.83 (p, J=7.3 Hz, 1H), 3.64 (p, J=6.9 Hz, 1H), 3.16 (s, 3H), 2.82 (dtt, J=8.6, 6.6, 2.4 Hz, 2H), 1.91 (dtt, J=9.2, 7.3, 2.4 Hz, 2H).

### Intermediate 116 (I-116)

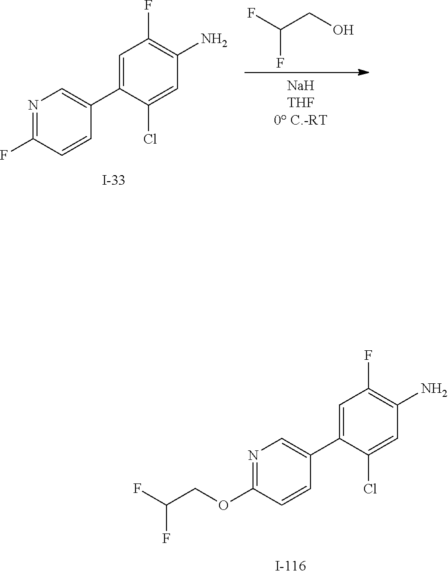

5-Chloro-4-(6-(2,2-difluoroethoxy)pyridin-3-yl)-2-fluoroaniline I-116 was synthesised from 5-chloro-2-fluoro-4-(6-fluoropyridin-3-yl)aniline I-33 and 2,2-difluoroethan-1-ol using a procedure essentially the same as for I-106. ¹H NMR (400 MHz, DMSO-d6) δ 8.17 (dd, J=2.6, 0.9 Hz, 1H), 7.80 (dd, J=8.6, 2.4 Hz, 1H), 7.13 (d, J=11.9 Hz, 1H), 6.97 (dd, J=8.6, 0.9 Hz, 1H), 6.91 (d, J=8.4 Hz, 1H), 6.41 (tt, J=54.7, 3.6 Hz, 1H), 5.62 (s, 2H), 4.59 (td, J=15.1, 3.6 Hz, 2H).

### Intermediate 117 (I-117)

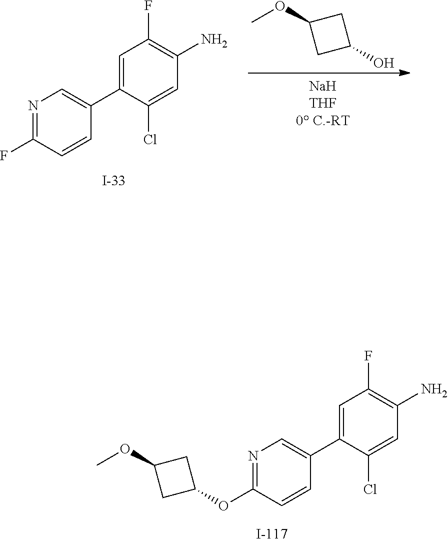

5-Chloro-2-fluoro-4-(6-((1r,3r)-3-methoxycyclobutoxy)pyridin-3-yl)aniline I-117 was synthesised from 5-chloro-2-fluoro-4-(6-fluoropyridin-3-yl)aniline I-33 and (1r,3r)-3-methoxycyclobutan-1-ol using a procedure essentially the same as for I-106. ¹H NMR (400 MHz, DMSO-d6) δ 8.17-8.07 (m, 1H), 7.77-7.67 (m, 1H), 7.11 (d, J=11.9 Hz, 1H), 6.90 (d, J=8.3 Hz, 1H), 6.83 (dd, J=8.5, 0.7 Hz, 1H), 5.60 (s, 2H), 5.26 (tt, J=7.1, 4.8 Hz, 1H), 4.07 (tt, J=6.9, 4.1 Hz, 1H), 3.17 (s, 3H), 2.40 (ddd, J=13.5, 7.1, 3.9 Hz, 2H), 2.36-2.24 (m, 2H).

### Intermediate 118 (I-118)

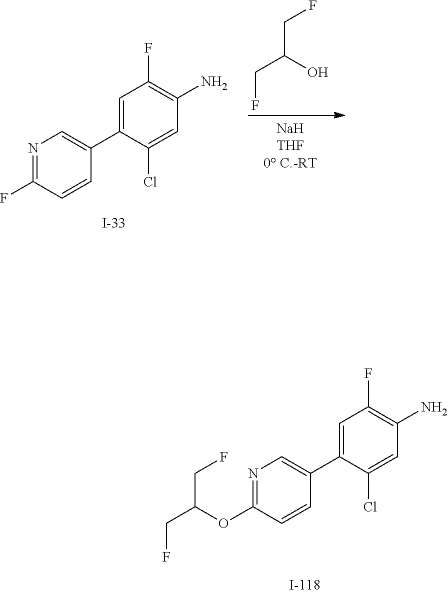

5-Chloro-4-(6-((1,3-difluoropropan-2-yl)oxy)pyridin-3-yl)-2-fluoroaniline I-118 was synthesised from 5-chloro-2-fluoro-4-(6-fluoropyridin-3-yl)aniline I-33 and (1,3-difluoropropan-2-ol using a procedure essentially the same as for I-106. ¹H NMR (400 MHz, DMSO-d6) δ 8.15 (dd, J=2.5, 0.8 Hz, 1H), 7.79 (dd, J=8.6, 2.6 Hz, 1H), 7.14 (d, J=11.9 Hz, 1H), 6.97-6.93 (m, 1H), 6.91 (d, J=8.4 Hz, 1H), 5.69-5.55 (m, 3H), 4.89-4.62 (m, 4H).

### Intermediate 119 (I-119)

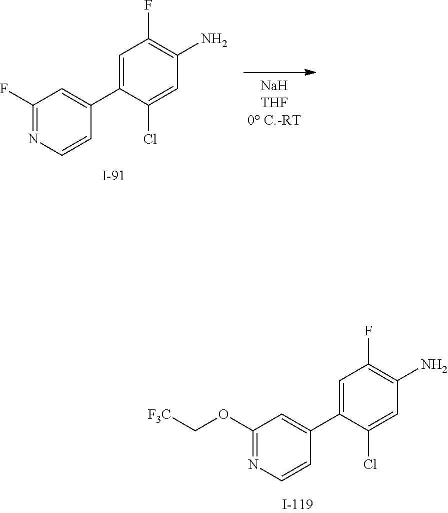

5-Chloro-2-fluoro-4-(2-(2,2,2-trifluoroethoxy)pyridin-4-yl)aniline I-119 was synthesised from 5-chloro-2-fluoro-4-(2-fluoropyridin-4-yl)aniline I-91 and trifluoroethanol using a procedure essentially the same as for I-106. ¹H NMR (400 MHz, DMSO-d6) δ 8.21 (dd, J=5.4, 0.7 Hz, 1H), 7.21 (d, J=12.0 Hz, 1H), 7.17 (dd, J=5.4, 1.5 Hz, 1H), 6.99 (dd, J=1.6, 0.7 Hz, 1H), 6.91 (d, J=8.3 Hz, 1H), 5.79 (s, 2H), 5.02 (q, J=9.2 Hz, 2H).

### Intermediate 120 (I-120)

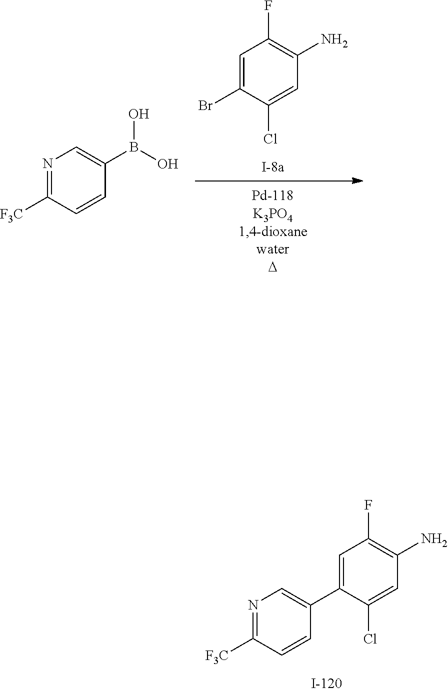 [SMILES: OB(O)c1ccc(C(F)(F)F)nc1 ; Nc1cc(Cl)c(Br)cc1F ; Nc1cc(Cl)c(-c2ccc(C(F)(F)F)nc2)cc1F]

5-Chloro-2-fluoro-4-(6-(trifluoromethyl)pyridin-3-yl)aniline I-120 was synthesised from

4-bromo-5-chloro-2-fluoroaniline I-8a and (6-(trifluoromethyl)pyridine-3-yl)boronic acid using a procedure essentially the same as for I-32. ¹H NMR (400 MHz, DMSO-d6) δ 8.79 (d, J=2.2 Hz, 1H), 8.12 (dd, J=8.1, 2.2 Hz, 1H), 7.95 (dd, J=8.2, 0.9 Hz, 1H), 7.30 (d, J=11.9 Hz, 1H), 6.95 (d, J=8.2 Hz, 1H), 5.81 (s, 2H).

### Intermediate 121 (I-121)

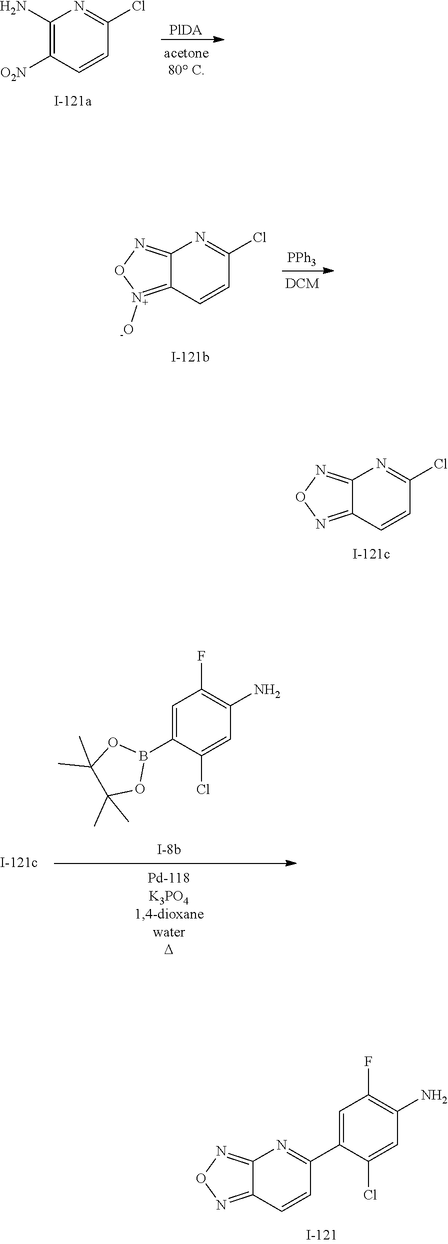

Step 1: To a solution of 6-Chloro-3-nitropyridin-2-ylamine I-121a (1.00 g, 5.76 mmol) in acetone (60 ml) was added PIDA (4.64 g, 14.4 mmol). The reaction mixture was heated to 80° C. for 6 h. The reaction was concentrated in vacuo. The product was purified by chromatography on silica gel (0-100% EtOAc/DCM) to afford 5-chloro-[1,2,5]oxadiazolo[3,4-b]pyridine 1-oxide I-121b as a yellow solid. ¹H NMR (400 MHz, CDCl₃) δ 7.77 (d, J=9.1 Hz, 1H), 7.16 (d, J=9.2 Hz, 1H).

Step 2: A solution of 5-chloro-[1,2,5]oxadiazolo[3,4-b]pyridine 1-oxide I-121b (403 mg, 2.35 mmol) in DCM (60 ml) was cooled at 0° C. and triphenylphosphine (924 mg, 3.52 mmol) was added. The reaction mixture was warmed to RT and stirred for 16 h. A 1 M aqueous NaOH solution (30 ml) was added and the mixture was vigorously stirred for 15 min. The product was extracted with DCM (3×50 ml). The combined organics were dried over sodium sulphate and concentrated in vacuo. The product was purified by chromatography on silica gel (0-50% EtOAc/isohexane). The product was further purified by chromatography on silica gel (0-30% EtOAc/isohexane) to afford 5-chloro-[1,2,5]oxadiazolo[3,4-b]pyridine I-121c as a yellow oil that solidified upon standing. ¹H NMR (400 MHz, CDCl₃) δ 8.22 (d, J=9.2 Hz, 1H), 7.40 (d, J=9.2 Hz, 1H)

Step 3: 4-([1,2,5]Oxadiazolo[3,4-b]pyridin-5-yl)-5-chloro-2-fluoroaniline I-121 was synthesised from 5-chloro-2-fluoro-4-(4,4,5,5-tetramethyl-1,3,2-dioxaborolan-2-yl)aniline I-8b and 5-chloro-[1,2,5]oxadiazolo[3,4-b]pyridine I-121c using a procedure essentially the same as for I-47. ¹H NMR (400 MHz, DMSO-d6) δ 8.57 (d, J=9.4 Hz, 1H), 7.99 (d, J=9.4 Hz, 1H), 7.53 (d, J=12.1 Hz, 1H), 6.95 (d, J=8.0 Hz, 1H), 6.21 (s, 2H).

### Intermediate 122 (I-122)

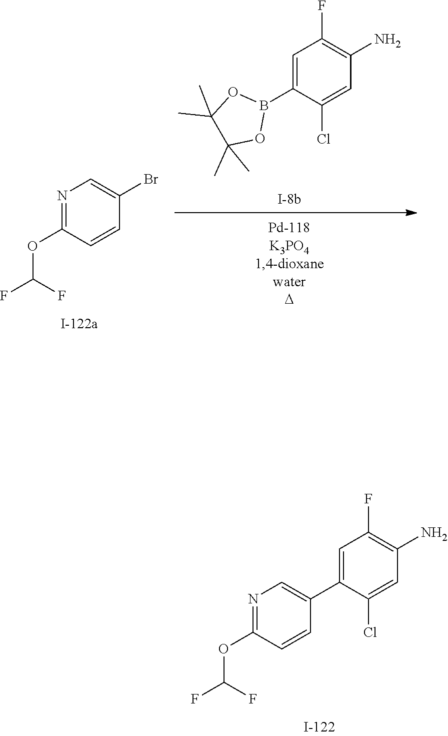 [SMILES: CC1(C)OB(c2cc(F)c(N)cc2Cl)OC1(C)C ; FC(F)Oc1ccc(Br)cn1 ; Nc1cc(Cl)c(-c2ccc(OC(F)F)nc2)cc1F]

5-Chloro-4-(6-(difluoromethoxy)pyridin-3-yl)-2-fluoroaniline I-122 was synthesised from 5-chloro-2-fluoro-4-(4,4,5,5-tetramethyl-1,3,2-dioxaborolan-2-yl)aniline I-8b and 5-bromo-2-(difluoromethoxy)pyridine I-122a using a procedure essentially the same as for I-47. ¹H NMR (400 MHz, DMSO-d6) δ 8.26 (dd, J=2.5, 0.7 Hz, 1H), 7.95 (dd, J=8.5, 2.5 Hz, 1H), 7.74 (t, J=72.9 Hz, 1H), 7.18 (d, J=11.9 Hz, 1H), 7.13 (dd, J=8.5, 0.7 Hz, 1H), 6.92 (d, J=8.3 Hz, 1H), 5.67 (s, 2H).

### Intermediate 123 (I-123)

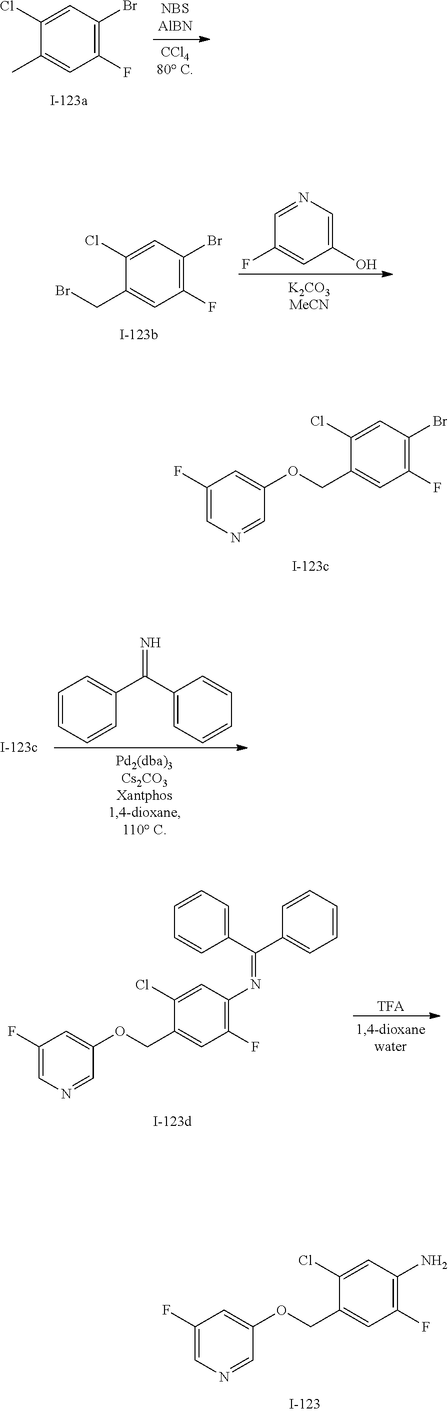

Step 1: To a solution of 1-bromo-5-chloro-2-fluoro-4-methylbenzene I-123a (3.7 g, 16.55 mmol) in CCl₄ (30 ml) were added NBS (3.2 g, 18.21 mmol) and AlBN (272 mg, 0.27 mmol) at RT. The reaction was then stirred at 80° C. for 16 h. Water (15 ml) was added and the mixture was extracted with EtOAc (3×15 ml). The combined organics were washed with brine (15 ml), dried over Na₂SO₄ and concentrated in vacuo to give 1-bromo-4-(bromomethyl)-5-chloro-2-fluorobenzene I-123b a white solid. ¹H NMR (400 MHz, DMSO-d6) δ 7.95 (d, J=6.4 Hz, 1H), 7.32 (d, J=8.8 Hz, 1H), 4.68 (s, 2H).

Step 2: To a solution of 1-bromo-4-(bromomethyl)-5-chloro-2-fluorobenzene I-123b (1.0 g, 3.30 mmol) in MeCN (10 ml) were added 5-fluoropyridin-3-ol (374 mg, 3.30 mmol) and K₂CO₃ (1.3 g, 9.92 mmol). The reaction was stirred at RT for 1 h. Water (10 ml) was added and the mixture was extracted with EtOAc (3×20 ml). The combined organics were washed with brine (15 ml), dried over Na₂SO₄ and concentrated in vacuo. The product was purified by chromatography on silica gel (10% EtOAc/petroleum ether) to give 3-((4-bromo-2-chloro-5-fluorobenzyl)oxy)-5-fluoropyridine I-123c as a yellow solid. ¹H NMR (400 MHz, DMSO-d6) δ 8.31 (s, 1H), 9.24 (d, J=2.0 Hz, 1H), 7.99 (d, J=6.0 Hz, 1H), 7.70 (d, J=9.2 Hz, 1H), 7.61 (dt, J=10.8, 2.4 Hz, 1H), 5.22 (s, 2H).

Step 3: To a solution of 3-((4-bromo-2-chloro-5-fluorobenzyl)oxy)-5-fluoropyridine I-123c (377 mg, 1.12 mmol) in 1,4-dioxane (3 ml) were added diphenylmethanimine (204 mg, 1.12 mmol), Pd₂(dba)₃ (116 mg, 0.11 mmol), Cs₂CO₃ (917 mg, 2.81 mmol) and Xantphos (130 mg, 0.22 mmol). The reaction was stirred at 110° C. for 3 h. Water (20 ml) was added and the mixture was extracted with EtOAc (3×20 ml). The combined organics were washed with brine (20 ml), dried over Na₂SO₄ and concentrated in vacuo. The product was purified by chromatography on silica gel (20% EtOAc/petroleum ether) to give N-(5-chloro-2-fluoro-4-(((5-fluoropyridin-3-yl)oxy)methyl)phenyl)-1,1-diphenylmethanimine I-123d as a white solid. LCMS (Method 3) m/z 434.9 (M+H)⁺ (ES⁺), at 2.44 min.

Step 4: To a solution of N-(5-chloro-2-fluoro-4-(((5-fluoropyridin-3-yl)oxy)methyl)phenyl)-1,1-diphenylmethanimine I-123d (72 mg, 0.16 mmol) in 1,4-dioxane (6 ml) and water (1.4 ml) was added TFA (14 drops). The reaction was stirred at RT for 10 min. The pH of the reaction mixture was adjusted to 8 by the addition of saturated aqueous NaHCO₃ and the product was extracted with EtOAc (3×5 ml). The combined organics were washed with brine (5 ml), dried over Na₂SO₄ and concentrated in vacuo to give 5-chloro-2-fluoro-4-(((5-fluoropyridin-3-yl)oxy)methyl)aniline) I-123 as a yellow solid. LCMS (Method 3) m/z 271 (M+H)⁺ (ES⁺), at 1.89 min.

### Intermediate 124 (I-124)

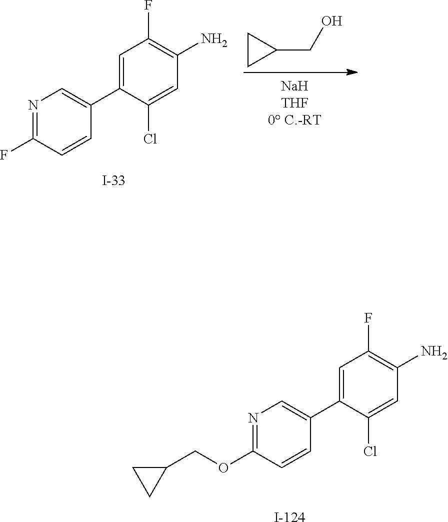

5-Chloro-4-(6-(cyclopropylmethoxy)pyridin-3-yl)-2-fluoroaniline I-124 was synthesised from 5-chloro-2-fluoro-4-(6-fluoropyridin-3-yl)aniline I-33 and cyclopropylmethanol using a procedure essentially the same as for I-106. ¹H NMR (400 MHz, DMSO-d6) δ 8.11 (dd, J=2.6, 0.8 Hz, 1H), 7.71 (dd, J=8.6, 2.5 Hz, 1H), 7.11 (d, J=11.9 Hz, 1H), 6.90 (d, J=8.4 Hz, 1H), 6.85 (dd, J=8.6, 0.7 Hz, 1H), 5.59 (s, 2H), 4.10 (d, J=7.1 Hz, 2H), 1.33-1.18 (m, 1H), 0.60-0.49 (m, 2H), 0.37-0.28 (m, 2H).

### Intermediate 125 (I-125)

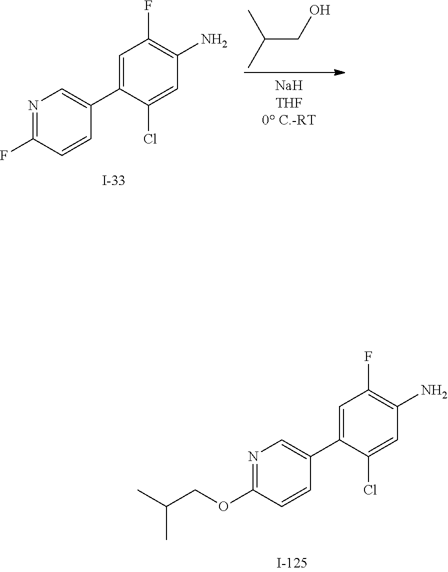

5-Chloro-2-fluoro-4-(6-isobutoxypyridin-3-yl)aniline I-125 was synthesised from 5-chloro-2-fluoro-4-(6-fluoropyridin-3-yl)aniline I-33 and 2-methylpropan-1-ol using a procedure essentially the same as for I-106. 1H NMR (400 MHz, DMSO-d6) δ 8.12 (dd, J=2.5, 0.7 Hz, 1H), 7.72 (dd, J=8.6, 2.6 Hz, 1H), 7.11 (d, J=11.9 Hz, 1H), 6.90 (d, J=8.4 Hz, 1H), 6.84 (dd, J=8.6, 0.8 Hz, 1H), 5.59 (s, 2H), 4.05 (d, J=6.6 Hz, 2H), 2.03 (dq, J=13.4, 6.7 Hz, 1H), 0.97 (d, J=6.7 Hz, 6H).

### Intermediate 126 (I-126)

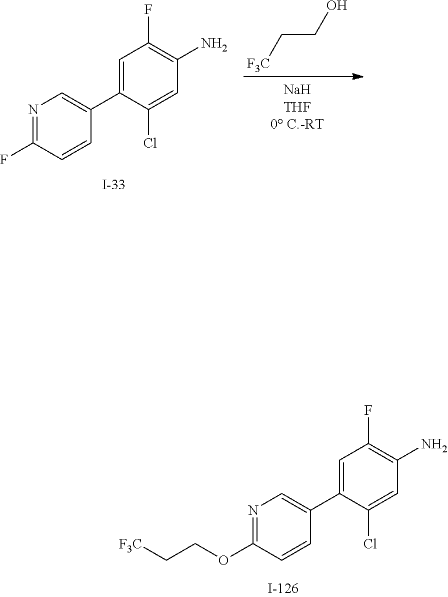

5-Chloro-2-fluoro-4-(6-(3,3,3-trifluoropropoxy)pyridin-3-yl)aniline I-126 was synthesised from 5-chloro-2-fluoro-4-(6-fluoropyridin-3-yl)aniline I-33 and 3,3,3-trifluoropropan-1-ol using a procedure essentially the same as for I-106. ¹H NMR (400 MHz, DMSO-d6) δ 8.16 (dd, J=2.5, 0.8 Hz, 1H), 7.76 (dd, J=8.6, 2.5 Hz, 1H), 7.13 (d, J=11.9 Hz, 1H), 6.91 (d, J=8.3 Hz, 1H), 6.87 (dd, J=8.5, 0.8 Hz, 1H), 5.61 (s, 2H), 4.51 (t, J=6.0 Hz, 2H), 2.81 (qt, J=11.6, 6.0 Hz, 2H).

### Intermediate 127 (I-127)

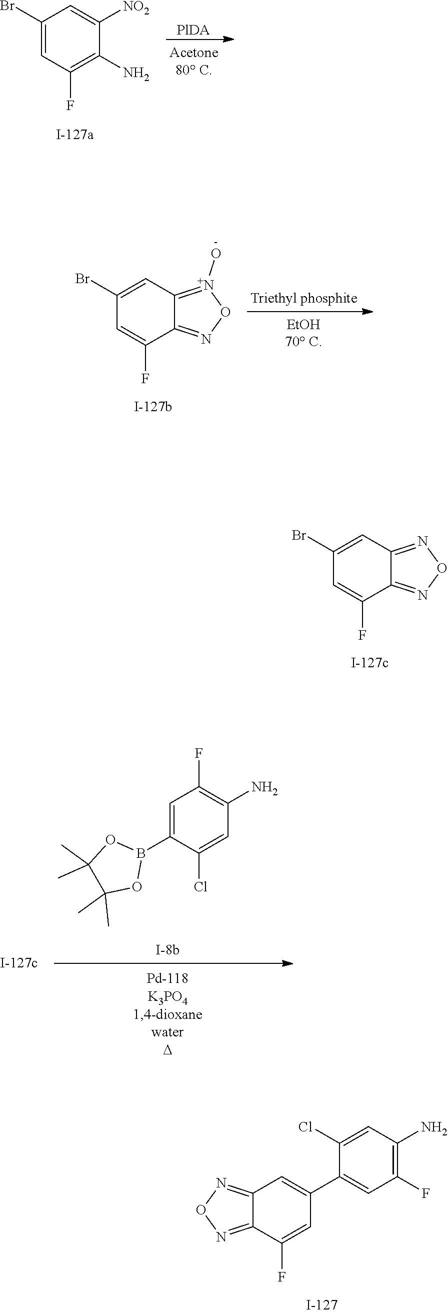 [SMILES: Nc1c(F)cc(Br)cc1[N+](=O)[O-] ; "PlDA" ; O[n+]1onc2c(F)cc(Br)cc21 ; Fc1cc(Br)cc2nonc12 ; CC1(C)OB(c2cc(F)c(N)cc2Cl)OC1(C)C ; Nc1cc(Cl)c(-c2cc(F)c3nonc3c2)cc1F]

Step 1: 6-Bromo-4-fluorobenzo[c][1,2,5]oxadiazole 1-oxide I-127b was synthesised from 4-bromo-2-fluoro-6-nitroaniline I-127a and PIDA using a procedure essentially the same as for I-121b. 1H NMR (400 MHz, DMSO-d6) δ 7.96 (s, 1H), 7.83-7.57 (m, 1H).

Step 2: To a solution of 5-bromo-7-fluorobenzo[c][1,2,5]oxadiazole 1-oxide I-127b (499 mg, 2.08 mmol) in EtOH (1.50 ml) was added triethyl phosphite (535 μl, 3.12 mmol) and the reaction mixture was heated to 70° C. for 2 h. The reaction was cooled to RT, diluted with DCM (15 ml) and 10% v/v aqueous sodium hypochlorite, then vigorously stirred for 20 min after which the dark brown solution passed through a phase separator and the filtrate was concentrated in vacuo. The product was purified by chromatography on silica gel (0-40% EtOAc/isohexane) to afford 6-bromo-4-fluorobenzo[c][1,2,5]oxadiazole I-127c as an orange oil that solidified over time to give an colourless oil.

Step 3: 6-Bromo-4-fluorobenzo[c][1,2,5]oxadiazole I-127 was synthesised from 5-chloro-2-fluoro-4-(4,4,5,5-tetramethyl-1,3,2-dioxaborolan-2-yl)aniline I-8b and 6-bromo-4-fluorobenzo[c][1,2,5]oxadiazole I-127c using a procedure essentially the same as for I-47. ¹H NMR (400 MHz, DMSO-d6) δ 7.92 (d, J=1.0 Hz, 1H), 7.63 (dd, J=11.5, 1.1 Hz, 1H), 7.33 (d, J=12.0 Hz, 1H), 6.94 (d, J=8.3 Hz, 1H), 5.87 (s, 2H).

### Intermediate 128 (I-128)

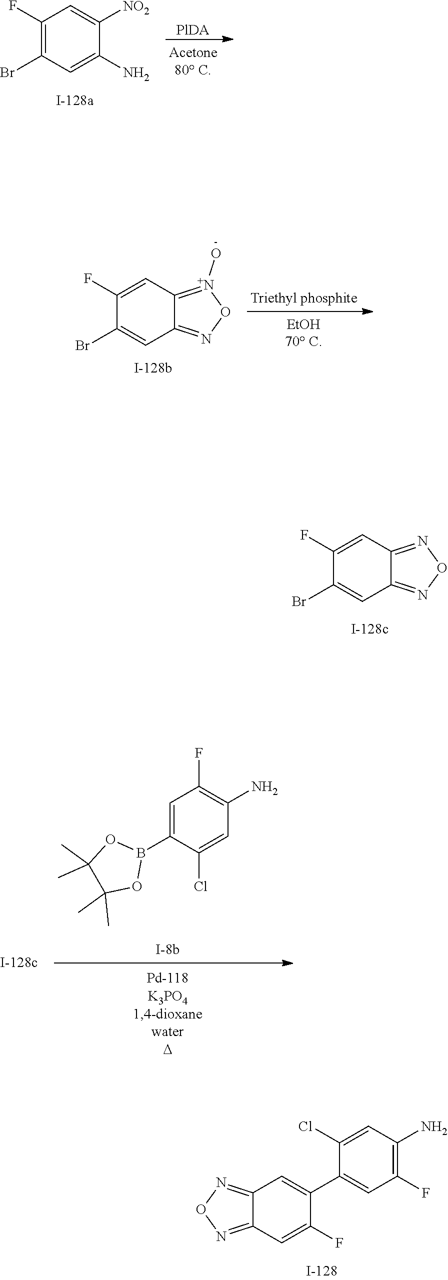 [SMILES: Nc1cc(Br)c(F)cc1[N+](=O)[O-] ; "PlDA" ; O[n+]1onc2cc(Br)c(F)cc21 ; Fc1cc2nonc2cc1Br ; CC1(C)OB(c2cc(F)c(N)cc2Cl)OC1(C)C ; Nc1cc(Cl)c(-c2cc3nonc3cc2F)cc1F]

Step 1: 5-Bromo-2-fluoro-4-(6-fluorobenzo[c][1,2,5]oxadiazol-5-yl)aniline I-128b was synthesised from 4-bromo-2-fluoro-6-nitroaniline I-128a and PIDA using a procedure essentially the same as for I-121b. ¹H NMR (400 MHz, DMSO-d6) δ 8.39 (s, 1H), 7.93 (s, 1H).

Step 2: 5-Bromo-2-fluoro-4-(6-fluorobenzo[c][1,2,5]oxadiazol-5-yl)aniline I-128c was synthesised from triethyl phosphite and 5-bromo-2-fluoro-4-(6-fluorobenzo[c][1,2,5]oxadiazol-5-yl)aniline I-128b using a procedure essentially the same as for I-121b. 1H NMR (400 MHz, DMSO-d6) δ 8.80 (dd, J=6.4, 0.6 Hz, 1H), 8.19 (dd, J=8.1, 0.6 Hz, 1H).

Step 3: 5-Chloro-2-fluoro-4-(6-fluorobenzo[c][1,2,5]oxadiazol-5-yl)aniline I-28 was synthesised from 5-chloro-2-fluoro-4-(4,4,5,5-tetramethyl-1,3,2-dioxaborolan-2-yl)aniline I-8b and 6-bromo-4-fluorobenzo[c][1,2,5]oxadiazole I-128b using a procedure essentially the same as for I-47. ¹H NMR (400 MHz, DMSO-d6) δ 8.17 (dd, J=6.7, 0.7 Hz, 1H), 8.05 (dd, J=9.2, 0.7 Hz, 1H), 7.27 (d, J=11.6 Hz, 1H), 6.95 (d, J=8.2 Hz, 1H), 5.83 (s, 2H).

### Intermediate 129 (I-129)

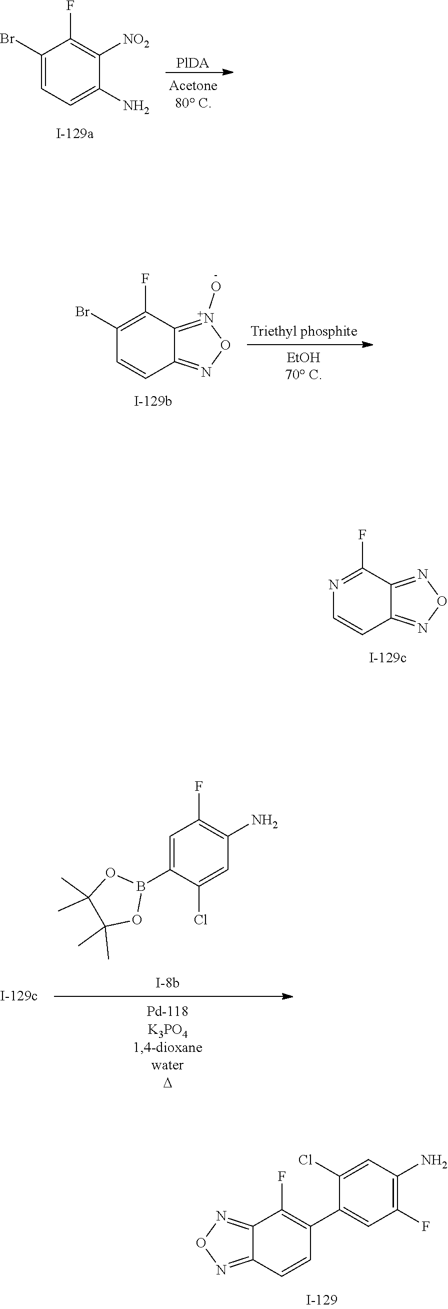 [SMILES: Nc1ccc(Br)c(F)c1[N+](=O)[O-] ; "PlDA" ; O[n+]1onc2ccc(Br)c(F)c21 ; Fc1nccc2nonc12 ; CC1(C)OB(c2cc(F)c(N)cc2Cl)OC1(C)C ; Nc1cc(Cl)c(-c2ccc3nonc3c2F)cc1F]

Step 1: 5-Bromo-4-fluorobenzo[c][1,2,5]oxadiazole 1-oxide I-129b was synthesised from 4-bromo-3-fluoro-2-nitroaniline I-129a and PIDA using a procedure essentially the same as for I-121b. ¹H NMR (400 MHz, CDCl₃) δ 7.28-7.22 (m, 1H), 7.14 (d, J=9.5 Hz, 1H).

Step 2: 5-Bromo-4-fluorobenzo[c][1,2,5]oxadiazole I-129c was synthesised from 5-Bromo-4-fluorobenzo[c][1,2,5]oxadiazole 1-oxide I-129b and triethyl phosphite using a procedure essentially the same as for I-121b. ¹H NMR (400 MHz, DMSO-d6) δ 7.95 (d, J=9.5 Hz, 1H), 7.82 (dd, J=9.4, 6.0 Hz, 1H).

Step 3: 5-chloro-2-fluoro-4-(4-fluorobenzo[c][1,2,5]oxadiazol-5-yl)aniline I-129 was synthesised from 5-chloro-2-fluoro-4-(4,4,5,5-tetramethyl-1,3,2-dioxaborolan-2-yl)aniline I-8b and 5-Bromo-4-fluorobenzo[c][1,2,5]oxadiazole I-129c using a procedure essentially the same as for I-47. ¹H NMR (400 MHz, DMSO-d6) δ 7.97 (d, J=9.2 Hz, 1H), 7.59 (dd, J=9.2, 6.3 Hz, 1H), 7.30 (d, J=11.7 Hz, 1H), 6.98 (d, J=8.2 Hz, 1H), 5.88 (s, 2H).

### Intermediate 130 (I-130)

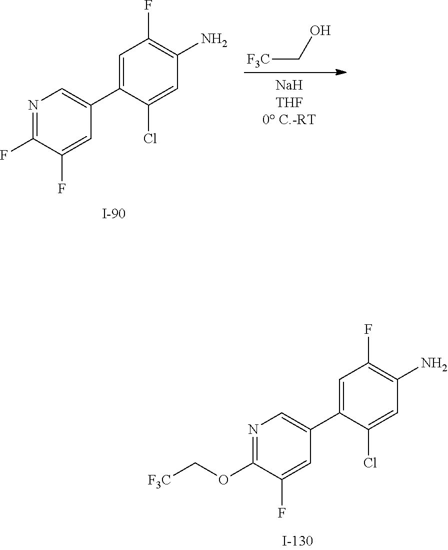

5-Chloro-2-fluoro-4-(5-fluoro-6-(2,2,2-trifluoroethoxy)pyridin-3-yl)aniline I-130 was synthesised from 5-chloro-4-(5,6-difluoropyridin-3-yl)-2-fluoroaniline I-90 and trifluoroethanol using a procedure essentially the same as for I-106. ¹H NMR (400 MHz, DMSO-d6) δ 8.04 (d, J=2.0 Hz, 1H), 7.90 (dd, J=11.4, 2.0 Hz, 1H), 7.20 (d, J=11.9 Hz, 1H), 6.92 (d, J=8.3 Hz, 1H), 5.69 (s, 2H), 5.12 (q, J=9.0 Hz, 2H).

### Intermediate 131 (I-131)

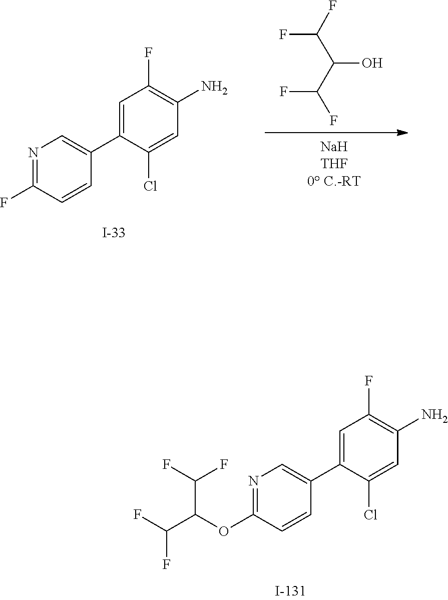

5-Chloro-2-fluoro-4-(6-((1,1,3,3-tetrafluoropropan-2-yl)oxy)pyridin-3-yl)aniline I-131 was synthesised from 5-chloro-2-fluoro-4-(6-fluoropyridin-3-yl)aniline I-33 and 1,1,3,3-tetrafluoropropan-2-ol using a procedure essentially the same as for I-106. 1H NMR (400 MHz, DMSO-d6) δ 8.19 (dd, J=2.5, 0.8 Hz, 1H), 7.86 (dd, J=8.5, 2.5 Hz, 1H), 7.17 (d, J=11.9 Hz, 1H), 7.06 (dd, J=8.6, 0.7 Hz, 1H), 6.91 (d, J=8.4 Hz, 1H), 6.70-6.33 (m, 2H), 6.07 (pt, J=12.6, 3.2 Hz, 1H), 5.65 (s, 2H).

### Intermediate 132 (I-132)

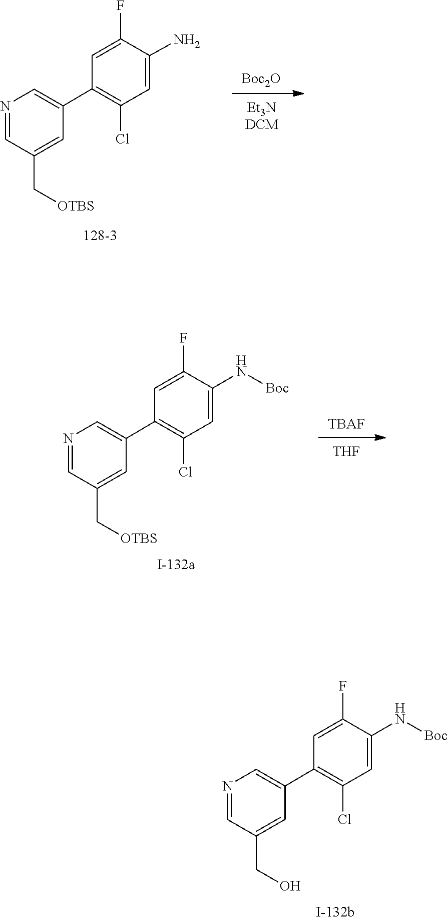

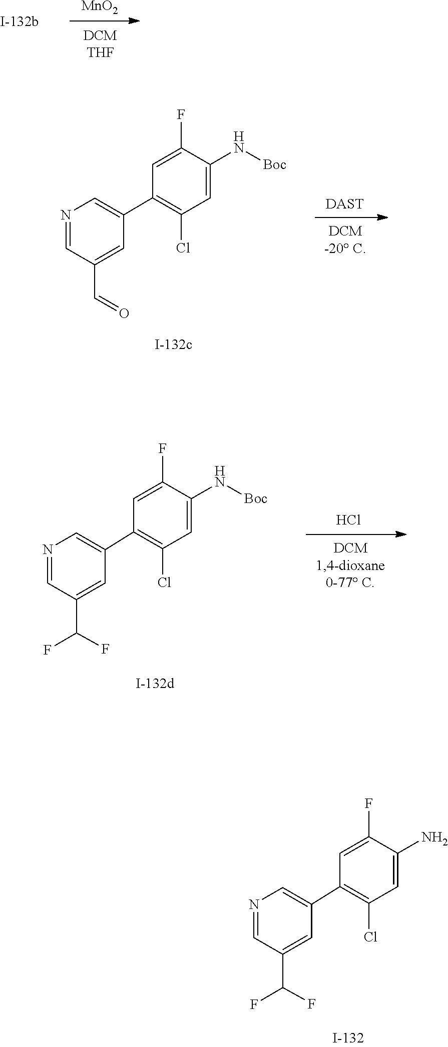

Step 1: To a solution of 4-(5-(((tert-butyldimethylsilyl)oxy)methyl)pyridin-3-yl)-5-chloro-2-fluoroaniline 128-3 (300 mg, 818 μmol) in DCM (4 ml) was added Et₃N (251 μl, 1.80 mmol) followed by di-tert-butyl dicarbonate (282 μl, 1.23 mmol). The reaction mixture was stirred at RT for 16 h. A further portion of Et₃N (114 μl, 818 μmol) and di-tert-butyl dicarbonate (94 μl, 409 μmol) were added and the reaction was further stirred at RT for 24 h. The reaction was diluted with DCM (15 ml) and washed with a saturated aqueous solution of NaHCO₃ (20 ml). The organics were passed through a hydrophobic frit and the filtrate was concentrated in vacuo. The product was purified by chromatography on silica gel (0-10% EtOAc/isohexane) to afford tert-butyl (4-(5-(((tert-butyldimethylsilyl)oxy)methyl)pyridin-3-yl)-5-chloro-2-fluorophenyl)carbamate I-132a as a colourless oil. ¹H NMR (400 MHz, DMSO-d6) δ 9.40 (s, 1H), 8.54 (dd, J=11.3, 2.2 Hz, 2H), 7.95 (d, J=7.3 Hz, 1H), 7.80 (t, J=2.2 Hz, 1H), 7.43 (d, J=11.2 Hz, 1H), 4.83 (s, 2H), 1.49 (s, 9H), 0.90 (s, 9H), 0.10 (s, 6H)

Step 2: A solution of tert-butyl (4-(5-(((tert-butyldimethylsilyl)oxy)methyl)pyridin-3-yl)-5-chloro-2-fluorophenyl)carbamate I-132a (201 mg, 430 μmol) in THF (5 ml) was cooled to 0° C. and a solution of TBAF in THF (646 μl, 1 M, 646 μmol) was added and the reaction mixture was slowly warmed to RT and stirred for 18 h. The reaction mixture was diluted with an aqueous saturated solution of NaHCO₃ (25 ml) and the product was extracted with DCM (3×10 ml). The combined organics were dried over MgSO₄ and concentrated in vacuo. The product was purified by chromatography on silica gel (0-50% EtOAc/isohexane) to afford tert-butyl (5-chloro-2-fluoro-4-(5-(hydroxymethyl)pyridin-3-yl)phenyl)carbamate I-132b as a white solid. ¹H NMR (400 MHz, DMSO-d6) δ 9.40 (s, 1H), 8.53 (dd, J=15.5, 2.1 Hz, 2H), 7.95 (d, J=7.3 Hz, 1H), 7.80 (t, J=2.2 Hz, 1H), 7.42 (d, J=11.1 Hz, 1H), 5.40 (t, J=5.7 Hz, 1H), 4.60 (d, J=5.8 Hz, 2H), 1.49 (s, 9H).

Step 3: To a solution of tert-butyl (5-chloro-2-fluoro-4-(5-(hydroxymethyl)pyridin-3-yl)phenyl)carbamate I-132b (141 mg, 332 μmol) in DCM (5 ml) and THF (2 ml) was added manganese dioxide (173 mg 1.99 mmol). The reaction mixture was stirred at RT for 24 h. The reaction was filtered on celite, the celite pad was rinsed with DCM and THF (50 ml). The filtrate was concentrated in vacuo to give tert-butyl (5-chloro-2-fluoro-4-(5-formylpyridine-3-yl)phenyl)carbamate I-132c as a white solid. ¹H NMR (400 MHz, DMSO-d6) δ 10.18 (s, 1H), 9.46 (s, 1H), 9.11 (d, J=2.0 Hz, 1H), 8.94 (d, J=2.3 Hz, 1H), 8.34 (t, J=2.1 Hz, 1H), 8.00 (d, J=7.2 Hz, 1H), 7.55 (d, J=11.2 Hz, 1H), 1.49 (s, 9H).

Step 4: To a suspension of tert-butyl (5-chloro-2-fluoro-4-(5-formylpyridine-3-yl)phenyl)carbamate I-132c (114 mg, 325 μmol) in DCM (10.0 ml) at −20° C. was slowly added DAST (172 μl, 1.30 mmol). The reaction was slowly warmed to RT and stirred at RT for 4 h. A saturated aqueous solution of NaHCO₃ (20 ml) was added and the product was extracted with DCM (3×15 ml). The combined organics were washed with brine (20 ml), dried with MgSO₄ and concentrated in vacuo. The product was purified by chromatography on silica gel (0-30% EtOAc/isohexane) to afford tert-butyl (5-chloro-4-(5-(difluoromethyl)pyridin-3-yl)-2-fluorophenyl)carbamate I-32d as a white solid. ¹H NMR (400 MHz, DMSO-d6) δ 9.44 (s, 1H), 8.86-8.77 (m, 2H), 8.13-8.08 (m, 1H), 7.99 (d, J=7.3 Hz, 1H), 7.52 (d, J=11.2 Hz, 1H), 7.22 (t, J=55.2 Hz, 1H), 1.49 (s, 9H).

Step 5: To a solution of tert-butyl (5-chloro-4-(5-(difluoromethyl)pyridin-3-yl)-2-fluorophenyl)carbamate (88.0 mg, 236 μmol) in DCM (3 ml) at 0° C. was added HCl in 1,4-dioxane (590 μl, 4 M, 2.36 mmol) and the reaction mixture was left to warm to RT and stirred at RT for 1 h. A further portion of HCl in 1,4-dioxane (3 mL, 4 M 12.0 mmol) was added and the reaction mixture was stirred at RT for 1 h. The reaction mixture was concentrated in vacuo to give a white solid. The material was dissolved in 1,4-dioxane (1 ml) and HCl in 1,4-dioxane (3 ml, 4 M, 12.0 mmol) was added. The reaction was stirred at RT for 2 h. The reaction was warmed to 30° C. for 16 h. The reaction mixture was heated up to 77° C. for 3 h. The reaction was cooled to RT and concentrated in vacuo to afford 5-chloro-4-(5-(difluoromethyl)pyridin-3-yl)-2-fluoroaniline I-132 as a yellow solid. ¹H NMR (400 MHz, DMSO-d6) δ 8.76 (d, J=3.3 Hz, 2H), 8.03 (s, 1H), 7.26 (d, J=11.9 Hz, 1H), 7.19 (t, J=55.2 Hz, 1H), 6.95 (d, J=8.3 Hz, 1H). 2 exchangeable protons not observed.

### Intermediate 133 (I-133)

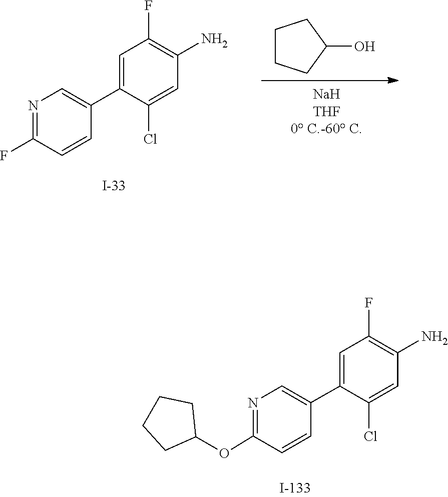

To a solution of cyclopentanol (107 mg, 1.25 mmol) in THF (3.0 ml) at 0° C. was added sodium hydride (53 mg, 60% w/w, 1.33 mmol) and the resulting suspension was stirred at RT for 30 min before 5-chloro-2-fluoro-4-(6-fluoropyridin-3-yl)aniline I-33 (200 mg, 831 μmol) in THF (3 ml) was added. The resulting suspension was heated to 60° C. for 2.5 h. The reaction mixture was cooled to RT, and water (0.1 ml) was added. Brine (25 ml) and DCM (25 ml) and the layers were separated. The aqueous phase was extracted with DCM (25 ml). The combined organics were dried over MgSO₄, and concentrated in vacuo. The product was purified by chromatography on silica gel (0-20% EtOAc/isohexane) to afford 5-chloro-4-(6-(cyclopentyloxy)pyridin-3-yl)-2-fluoroaniline I-133 as a yellow oil. ¹H NMR (400 MHz, DMSO-d6) δ 8.13 (dd, J=2.6, 0.7 Hz, 1H), 7.69 (dd, J=8.6, 2.5 Hz, 1H), 7.11 (d, J=11.9 Hz, 1H), 6.90 (d, J=8.3 Hz, 1H), 6.77 (dd, J=8.5, 0.7 Hz, 1H), 5.58 (s, 2H), 5.48-5.31 (m, 1H), 1.99-1.86 (m, 2H), 1.82-1.66 (m, 4H), 1.66-1.45 (m, 2H).

### Intermediate 134 (I-134)

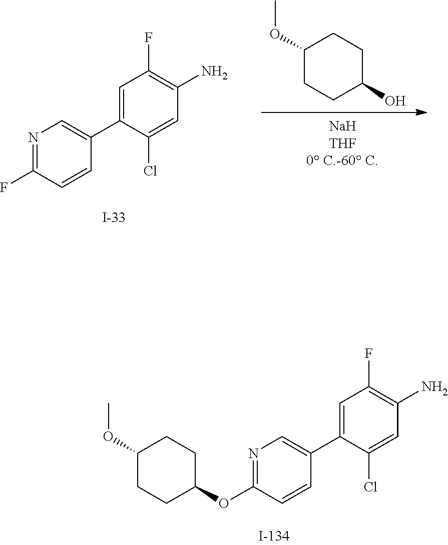

5-Chloro-2-fluoro-4-(6-(((1r,4r)-4-methoxycyclohexyl)oxy)pyridin-3-yl)aniline I-134 was synthesised from 5-chloro-2-fluoro-4-(6-fluoropyridin-3-yl)aniline I-33 and (1r,4r)-4-methoxycyclohexan-1-ol using a procedure essentially the same as for I-133. ¹H NMR (400 MHz, DMSO-d6) δ 8.12 (dd, J=2.5, 0.7 Hz, 1H), 7.70 (dd, J=8.6, 2.6 Hz, 1H), 7.10 (d, J=11.9 Hz, 1H), 6.90 (d, J=8.3 Hz, 1H), 6.78 (dd, J=8.5, 0.7 Hz, 1H), 5.58 (s, 2H), 5.10-4.94 (m, 1H), 3.25 (s, 4H), 2.14-1.89 (m, 4H), 1.62-1.40 (m, 2H), 1.42-1.26 (m, 2H).

### Intermediate 135 (I-135)

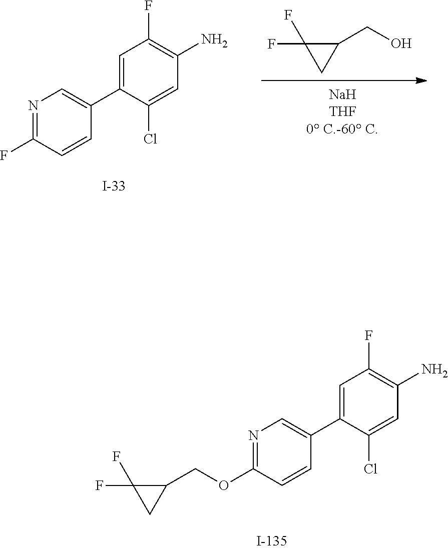

5-Chloro-4-(6-((2,2-difluorocyclopropyl)methoxy)pyridin-3-yl)-2-fluoroaniline I-135 was synthesised from 5-chloro-2-fluoro-4-(6-fluoropyridin-3-yl)aniline I-33 and (2,2-difluorocyclopropyl)methanol using a procedure essentially the same as for I-133. ¹H NMR (400 MHz, DMSO-d6) δ 8.14 (dd, J=2.6, 0.8 Hz, 1H), 7.75 (dd, J=8.6, 2.5 Hz, 1H), 7.12 (d, J=11.9 Hz, 1H), 6.99-6.78 (m, 2H), 5.59 (s, 2H), 4.61-4.39 (m, 1H), 4.22 (ddd, J=11.7, 8.6, 1.8 Hz, 1H), 2.36-2.14 (m, 1H), 1.79-1.71 (m, 1H), 1.53-1.46 (m, 1H).

### Intermediate 136 (I-136)

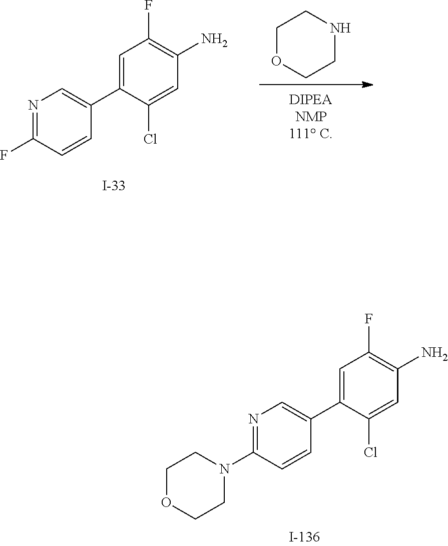

To a solution of 5-chloro-2-fluoro-4-(6-fluoropyridin-3-yl)aniline I-33 (200 mg, 831 μmol) in NMP (5 ml) was added DIPEA (316 μl, 1.83 mmol) followed by morpholine (109 μl, 1.25 mmol). The reaction mixture was heated to 56° C. for 1 h. The reaction was heated to 88° C. for 1 h. The reaction was heated to 111° C. for 16 h. The reaction mixture was diluted with water (25 ml) and the product was extracted with DCM (3×25 ml). The combined organics were dried with MgSO₄ and concentrated in vacuo. The product was purified by chromatography on silica gel (0-30% EtOAc/isohexane) to afford product containing ˜20% NMP. The material was dissolved in EtOAc (25 ml), washed with water (4×25 ml), 1 M aqueous LiCl (20 ml) and brine (20 ml). The organics were dried over MgSO₄ and concentrated in vacuo to give 5-chloro-2-fluoro-4-(6-morpholinopyridin-3-yl)aniline I-136 as a yellow oil that solidified upon standing to from a yellow solid. ¹H NMR (400 MHz, DMSO-d6) δ 8.15-8.10 (m, 1H), 7.59 (dd, J=8.8, 2.5 Hz, 1H), 7.07 (d, J=12.0 Hz, 1H), 6.92-6.82 (m, 2H), 5.52 (s, 2H), 3.70 (dd, J=5.7, 4.0 Hz, 4H), 3.47 (dd, J=5.7, 4.1 Hz, 4H).

### Intermediate 137 (I-137)

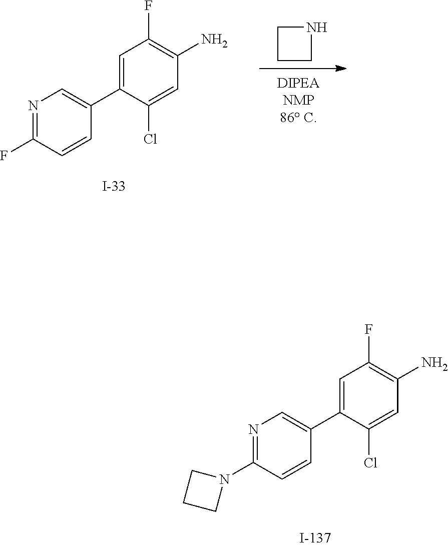

To a solution of 5-chloro-2-fluoro-4-(6-fluoropyridin-3-yl)aniline I-33 (200 mg, 831 μmol) in NMP (5 ml) was added DIPEA (316 μl, 1.83 mmol) was added followed by azetidine (84 μl, 1.25 mmol). The reaction was heated to 55° C. for 2 h then 86° C. for 1 h. The reaction mixture was cooled to RT. Water (30 ml) was added and the precipitate was isolated by filtration and the solid was dried in vacuo to give 4-(6-(azetidin-1-yl)pyridin-3-yl)-5-chloro-2-fluoroaniline as a colourless solid. ¹H NMR (400 MHz, DMSO-d6) δ 8.04 (dd, J=2.5, 0.8 Hz, 1H), 7.53 (dd, J=8.6, 2.4 Hz, 1H), 7.04 (d, J=12.0 Hz, 1H), 6.88 (d, J=8.5 Hz, 1H), 6.37 (dd, J=8.5, 0.8 Hz, 1H), 5.50 (s, 2H), 3.95 (t, J=7.4 Hz, 4H), 2.33 (p, J=7.4 Hz, 2H).

### Intermediate 138 (I-138)

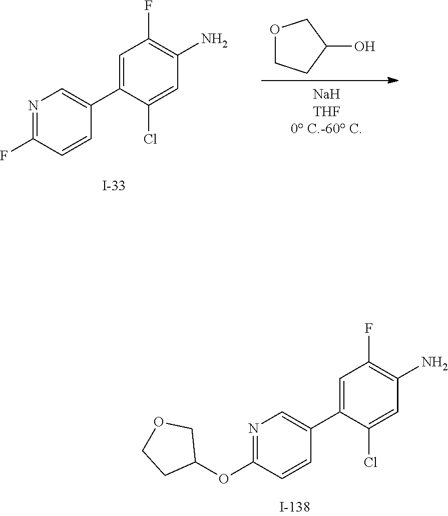

5-Chloro-2-fluoro-4-(6-((tetrahydrofuran-3-yl)oxy)pyridin-3-yl)aniline I-138 was synthesised from 5-chloro-2-fluoro-4-(6-fluoropyridin-3-yl)aniline I-33 and tetrahydrofuran-3-ol using a procedure essentially the same as for I-133. ¹H NMR (400 MHz, DMSO-d6) δ 8.14 (dd, J=2.6, 0.7 Hz, 1H), 7.74 (dd, J=8.5, 2.5 Hz, 1H), 7.12 (d, J=11.9 Hz, 1H), 6.91 (d, J=8.4 Hz, 1H), 6.85 (dd, J=8.6, 0.8 Hz, 1H), 5.59 (s, 2H), 5.52 (ddt, J=6.6, 4.3, 2.0 Hz, 1H), 3.93 (dd, J=10.2, 4.7 Hz, 1H), 3.86 (td, J=8.2, 6.9 Hz, 1H), 3.80-3.71 (m, 2H), 2.30-2.16 (m, 1H), 2.08-2.00 (m, 1H).

### Intermediate 139 (I-139)

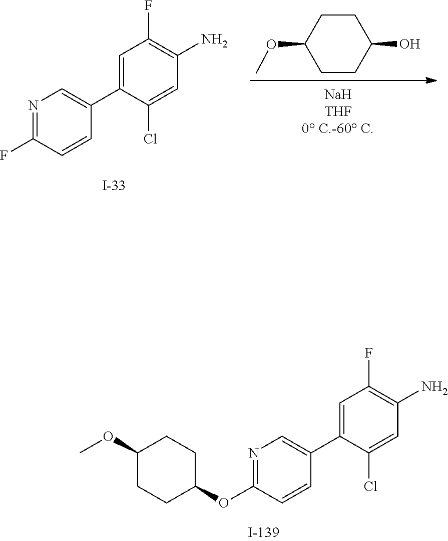

5-Chloro-2-fluoro-4-(6-(((1s,4s)-4-methoxycyclohexyl)oxy)pyridin-3-yl)aniline I-139 was synthesised from 5-chloro-2-fluoro-4-(6-fluoropyridin-3-yl)aniline I-33 and (1s,4s)-4-methoxycyclohexan-1-ol using a procedure essentially the same as for I-133. ¹H NMR (400 MHz, DMSO-d6) δ 8.11 (dd, J=2.6, 0.7 Hz, 1H), 7.70 (dd, J=8.6, 2.6 Hz, 1H), 7.11 (d, J=11.9 Hz, 1H), 6.90 (d, J=8.4 Hz, 1H), 6.79 (dd, J=8.5, 0.7 Hz, 1H), 5.58 (s, 2H), 5.07 (dt, J=7.8, 4.1 Hz, 1H), 3.34-3.28 (m, 4H), 1.85-1.54 (m, 8H)

### Experimental Scheme 2

Compound 15: (±)—N-(5-Bromo-4-chloro-2-fluorophenyl)-2-oxo-3,5,6,7,8,9-hexahydro-2H-5,8-epiminocyclohepta[d]pyrimidine-10-carboxamide

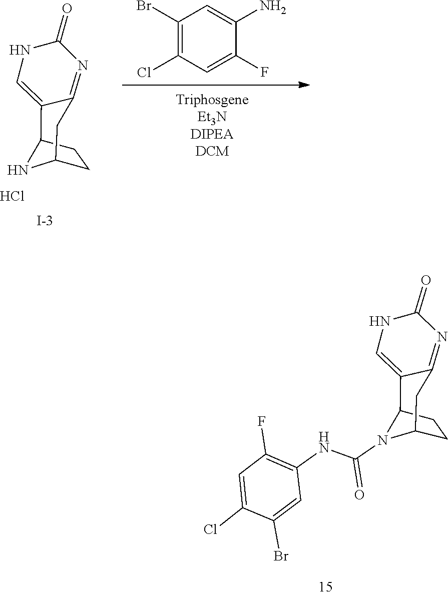

A solution of triphosgene (53 mg, 0.18 mmol) and 5-bromo-4-chloro-2-fluoroaniline (89 mg, 0.39 mmol) in DCM (4 ml) at RT was treated with Et₃N (0.16 ml, 1.2 mmol). The mixture was stirred at RT for 10 min. The resulting solution was added to a pre-sonicated suspension of (±)-6,7,8,9-tetrahydro-5H-5,8-epiminocyclohepta[d]pyrimidin-2-ol, HCl I-3 (86 mg, 0.39 mmol) and DIPEA (0.21 ml, 1.2 mmol) in DCM (4 ml). The reaction mixture was stirred at RT for 3 h. The reaction mixture was treated with MTBE (20 ml) and left to settle for 72 h. The resultant suspension was filtered and the solid was washed with MTBE. The solids were dissolved in MeOH (100 ml) and treated with MP-carbonate resin (1 g, 3 mmol) and the suspension was stirred at RT for 18 h. The suspension was filtered and the filtrate was concentrated in vacuo to afford (±)—N-(5-bromo-4-chloro-2-fluorophenyl)-2-hydroxy-6,7,8,9-tetrahydro-5H-5,8-epiminocyclohepta[d]pyrimidine-10-carboxamide 15 as an off-white solid. LCMS (Method 1) m/z 427.0, 429.0 (M+H)⁺ (ES⁺), at 0.84 min, ¹H NMR (400 MHz, DMSO-d6) δ 11.63 (br s, 1H), 8.74 (s, 1H), 7.99-7.61 (m, 3H), 5.05 (d, J=5.8 Hz, 1H), 4.72-4.57 (m, 1H), 3.12 (dd, J=18.7, 5.1 Hz, 1H), 2.26-2.16 (m, 1H), 2.12-2.02 (m, 1H), 1.84-1.67 (m, 2H), 1 CH under DMSO-d6 peak

The following compounds were prepared using appropriate starting materials in an analogous procedure to that described in Experimental Scheme 2

| | | Method | |
|---|---|---|---|
| Com- | | LCMS Rt | |
| pound \| | Structure | [M + H]⁺ | NMR |
| 16^{(b)(c)} | 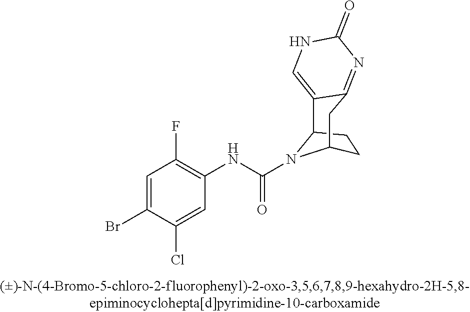 [SMILES: O=C(Nc1cc(Cl)c(Br)cc1F)N1C2CCC1c1c[nH]c(=O)nc1C2] | Method 1 426.7, 429.1 at 0.88 min | ¹H NMR (400 MHz, DMSO-d6) δ 11.70 (br s, 1H), 8.75 (s, 1H), 7.93-7.70 (m, 3H), 5.06 (d, J = 5.8 Hz, 1H), 4.64 (t, J = 6.5 Hz, 1H), 3.16-3.08 (m, 1H), 2.26-2.15 (m, 1H), 2.12-2.01 (m, 1H), 1.83-1.68 (m, 2H). 1 CH under DMSO-d6 peak |
| 17 | 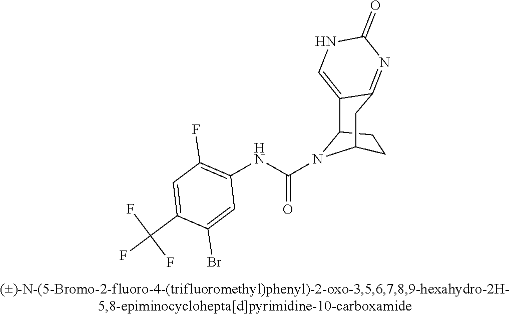 [SMILES: O=C(Nc1cc(Br)c(C(F)(F)F)cc1F)N1C2CCC1c1c[nH]c(=O)nc1C2] | Method 1 460.8, 463.2 at 0.99 min | ¹H NMR (400 MHz, DMSO-d6) δ 8.20 (d, J = 7.2 Hz, 1H), 7.94 (s, 1H), 7.75 (d, J = 11.2 Hz, 1H), 5.09 (d, J = 5.8 Hz, 1H), 4.74-4.62 (m, 1H), 3.13 (dd, J = 18.8, 4.5 Hz, 1H), 2.58-2.52 (m, 1H), 2.26-2.16 (m, 1H), 2.14-2.01 (m, 1H), 1.84-1.66 (m, 2H) Pyrimidone NH not observed |
| 54^{(c)} | 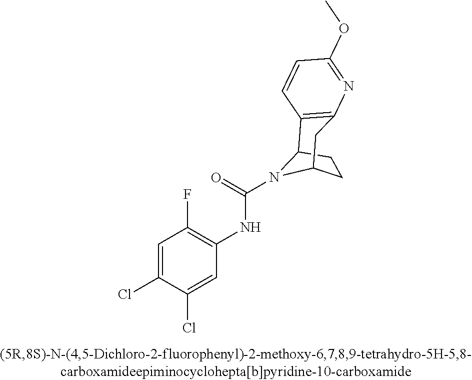 [SMILES: COc1ccc2c(n1)CC1CCC2N1C(=O)Nc1cc(Cl)c(Cl)cc1F] | Method 1 396.1, 398.2 at 1.65 min | ¹H NMR (400 MHz, DMSO-d6) δ 8.71 (s, 1H), 7.83 (d, J = 7.6 Hz, 1H), 7.66 (d, J = 10.3 Hz, 1H), 7.47 (d, J = 8.3 Hz, 1H), 6.58 (d, J = 8.3 Hz, 1H), 5.11 (d, J = 5.8 Hz, 1H), 4.71 (t, J = 6.2 Hz, 1H), 3.78 (s, 3H), 3.32-3.25 (m, 1H), 2.61 (d, J = 17.6 Hz, 1H), 2.29-2.06 (m, 2H), 1.84-1.73 (m, 1H), 1.70 (dt, J = 15.7, 7.8 Hz, 1H) |
| 55^{(b)(c)} | 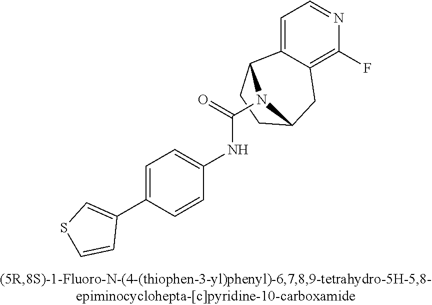 [SMILES: O=C(Nc1ccc(-c2ccsc2)cc1)N1[C@H]2CC[C@@H]1c1ccnc(F)c1C2] | Method 1 380.3 at 1.50 min | ¹H NMR (400 MHz, DMSO-d6) δ 8.79 (s, 1H), 8.01 (d, J = 5.0 Hz, 1H), 7.72 (dd, J = 2.9, 1.3 Hz, 1H), 7.61-7.56 (m, 3H), 7.52-7.45 (m, 3H), 7.21 (dd, J = 5.0, 1.7 Hz, 1H), 5.25 (d, J = 6.0 Hz, 1H), 4.82 (t, J = 6.2 Hz, 1H), 3.22-3.11 (m, 1H), 2.58 (d, J = 17.4 Hz, 1H), 2.31-2.14 (m, 2H), 1.89-1.80 (m, 1H), 1.80-1.71 (m, 1H). |
| 56^{(b)(c)} | 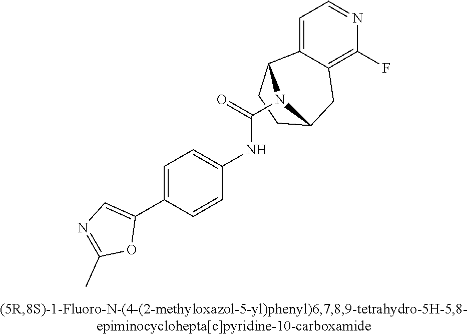 [SMILES: Cc1ncc(-c2ccc(NC(=O)N3[C@H]4CC[C@@H]3c3ccnc(F)c3C4)cc2)o1] | Method 1 379.1 at 1.20 min | ¹H NMR (400 MHz, DMSO-d6) δ 8.88 (s, 1H), 8.01 (d, J = 5.0 Hz, 1H), 7.58-7.48 (m, 4H), 7.35 (s, 1H), 7.20 (dd, J = 5.0, 1.7 Hz, 1H), 5.25 (d, J = 6.0 Hz, 1H), 4.86-4.75 (m, 1H), 3.16 (dd, J = 17.4, 4.9 Hz, 1H), 2.58 (d, J = 17.3 Hz, 1H), 2.44 (s, 3H), 2.31-2.14 (m, 2H), 1.89-1.70 (m, 2H). |
| 57^{(b)(c)} | 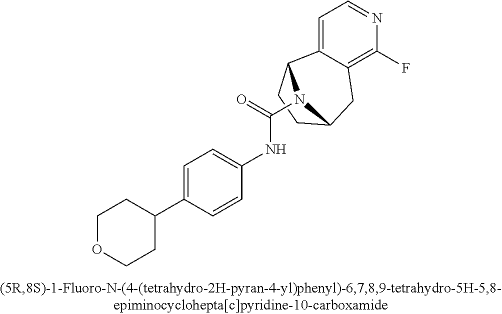 [SMILES: O=C(Nc1ccc(C2CCOCC2)cc1)N1[C@H]2CC[C@@H]1c1ccnc(F)c1C2] | Method 1 382.4 at 1.26 min | ¹H NMR (500 MHz, DMSO-d6) δ 8.67 (s, 1H), 8.00 (d, J = 5.0 Hz, 1H), 7.37-7.33 (m, 2H), 7.19 (dd, J = 5.1, 1.6 Hz, 1H), 7.14-7.05 (m, 2H), 5.21 (d, J = 6.2 Hz, 1H), 4.83-4.73 (m, 1H), 3.95-3.84 (m, 2H), 3.39 (td, J = 11.3, 3.0 Hz, 2H), 3.14 (dd, J = 17.3, 5.0 Hz, 1H), 2.70-2.60 (m, 1H), 2.56 (d, J = 17.3 Hz, 1H), 2.19 (dtd, J = 23.8, 11.7, 7.0 Hz, 2H), 1.86-1.80 (m, 1H), 1.78-1.70 (m, 1H), 1.67-1.53 (m, 4H). |
| 58^{(b)(c)} | 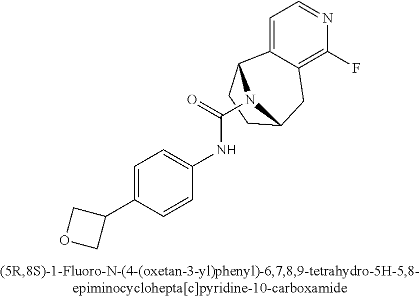 [SMILES: O=C(Nc1ccc(C2COC2)cc1)N1[C@H]2CC[C@@H]1c1ccnc(F)c1C2] | Method 1 354.3 at 1.08 min | ¹H NMR (500 MHz, DMSO-d6) δ 8.74 (s, 1H), 8.00 (d, J = 5.0 Hz, 1H), 7.45-7.40 (m, 2H), 7.28-7.23 (m, 2H), 7.19 (dd, J = 5.0, 1.6 Hz, 1H), 5.23 (d, J = 6.3 Hz, 1H), 4.89 (dd, J = 8.4, 5.8 Hz, 2H), 4.79 (t, J = 6.3 Hz, 1H), 4.55 (dd, J = 6.9, 5.8 Hz, 2H), 4.15 (ddd, J = 15.2, 8.4, 6.9 Hz, 1H), 3.14 (dd, J = 17.4, 5.0 Hz, 1H), 2.57 (d, J = 17.3 Hz, 1H), 2.29-2.12 (m, 2H), 1.89-1.79 (m, 1H), 1.79-1.67 (m, 1H). |
| 59^{(b)(c)} | 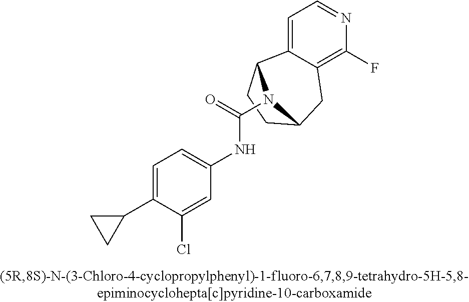 [SMILES: O=C(Nc1ccc(C2CC2)c(Cl)c1)N1[C@H]2CC[C@@H]1c1ccnc(F)c1C2] | Method 1 372.4 at 1.60 min | ¹H NMR (400 MHz, DMSO-d6) δ 8.80 (s, 1H), 8.00 (d, J = 5.0 Hz, 1H), 7.61 (d, J = 2.3 Hz, 1H), 7.34-7.11 (m, 2H), 6.89 (d, J = 8.6 Hz, 1H), 5.21 (d, J = 6.1 Hz, 1H), 4.78 (t, J = 6.1 Hz, 1H), 3.13 (dd, J = 17.4, 5.0 Hz, 1H), 2.57 (d, J = 17.3 Hz, 1H), 2.28-2.09 (m, 2H), 2.01 (ddd, J = 13.7, 8.5, 5.3 Hz, 1H), 1.91-1.64 (m, 2H), 0.98-0.83 (m, 2H), 0.64-0.53 (m, 2H). |
| 61^{(b)(c)} | 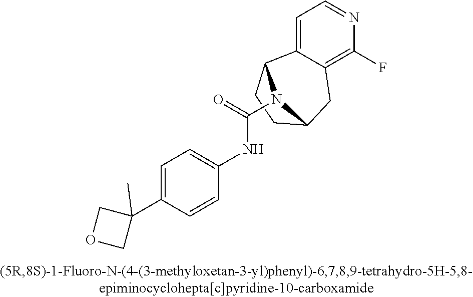 [SMILES: CC1(c2ccc(NC(=O)N3[C@H]4CC[C@@H]3c3ccnc(F)c3C4)cc2)COC1] | Method 1 368.3 at 1.17 min | ¹H NMR (400 MHz, DMSO-d6) δ 8.73 (s, 1H), 8.00 (d, J = 5.0 Hz, 1H), 7.45-7.37 (m, 2H), 7.19 (dd, J = 5.0, 1.7 Hz, 1H), 7.13-7.06 (m, 2H), 5.22 (d, J = 6.0 Hz, 1H), 4.83-4.77 (m, 1H), 4.74 (d, J = 5.5 Hz, 2H), 4.48 (d, J = 5.6 Hz, 2H), 3.15 (dd, J = 17.4, 5.0 Hz, 1H), 2.56 (d, J = 17.4 Hz, 1H), 2.30-2.13 (m, 2H), 1.88-1.80 (m, 1H), 1.79-1.70 (m, 1H), 1.57 (s, 3H). |
| 62^{(b)(e)} | 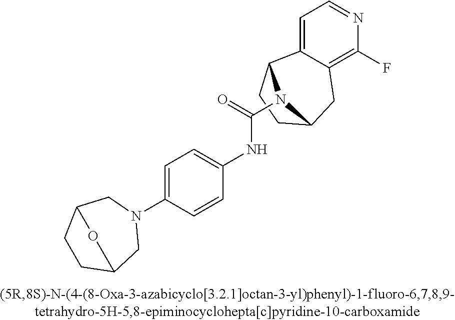 [SMILES: O=C(Nc1ccc(N2CC3CCC(C2)O3)cc1)N1[C@H]2CC[C@@H]1c1ccnc(F)c1C2] | Method 1 409.2 at 1.23 min | ¹H NMR (400 MHz, DMSO-d6) δ 8.47 (s, 1H), 7.99 (d, J = 5.0 Hz, 1H), 7.26-7.20 (m, 2H), 7.18 (dd, J = 5.0, 1.7 Hz, 1H), 6.74-6.68 (m, 2H), 5.19 (d, J = 6.0 Hz, 1H), 4.78-4.71 (m, 1H), 4.38 (s, 2H), 3.30-3.24 (m, 2H), 3.13 (dd, J = 17.5, 5.0 Hz, 1H), 2.70 (dd, J = 11.5, 2.5 Hz, 2H), 2.56 (s, 1H), 2.28-2.12 (m, 2H), 1.87-1.77 (m, 5H), 1.78-1.65 (m, 1H). |
| 63^{(b)(c)} | 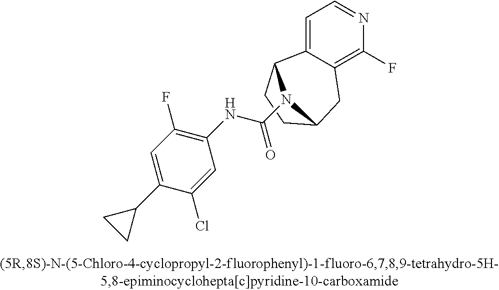 [SMILES: O=C(Nc1cc(Cl)c(C2CC2)cc1F)N1[C@H]2CC[C@@H]1c1ccnc(F)c1C2] | Method 1 390.3, 392.1 at 1.62 min | ¹H NMR (400 MHz, DMSO-d6) δ 8.62 (s, 1H), 8.00 (d, J = 5.0 Hz, 1H), 7.55-7.49 (m, 1H), 7.21-7.15 (m, 1H), 6.91-6.83 (m, 1H), 5.18 (d, J = 6.0 Hz, 1H), 4.75 (t, J = 6.2 Hz, 1H), 3.17 (dd, J = 17.2, 4.9 Hz, 1H), 2.56 (d, J = 17.3 Hz, 1H), 2.30-2.11 (m, 2H), 2.11-2.00 (m, 1H), 1.88-1.78 (m, 1H), 1.79-1.69 (m, 1H), 1.01-0.92 (m, 2H), 0.73-0.64 (m, 2H) |
| 64^{(a)(d)} | 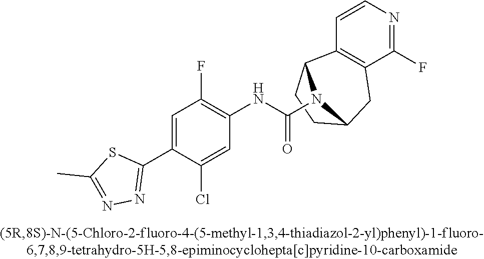 [SMILES: Cc1nnc(-c2cc(F)c(NC(=O)N3[C@H]4CC[C@@H]3c3ccnc(F)c3C4)cc2Cl)s1] | Method 3 448.1 at 3.20 min | ¹H NMR (400 MHz, DMSO-d6) δ 9.01 (s, 1H), 8.01-7.96 (m, 3H), 7.21 (d, J = 4.8 Hz, 1H), 5.26 (d, J = 6 Hz, 1H), 4.83-4.81 (m, 1H), 3.22-3.17 (m, 1H), 2.79 (s, 3H), 2.62-2.58 (m, 1H), 2.32-2.16 (m, 2H), 2.00-1.72 (m, 2H). |
| 65^{(a)(d)} | 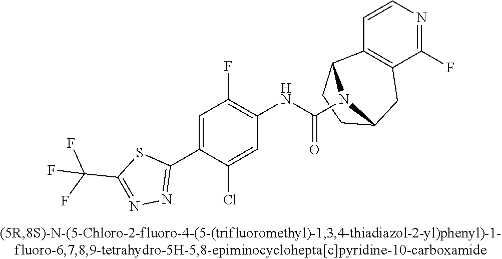 [SMILES: O=C(Nc1cc(Cl)c(-c2nnc(C(F)(F)F)s2)cc1F)N1[C@H]2CC[C@@H]1c1ccnc(F)c1C2] | Method 3 502.1 at 2.09 min | ¹H NMR (400 MHz, DMSO-d6) δ 9.14 (s, 1H), 8.18 (d, J = 11.6 Hz, 1H), 8.10 (d, J = 7.2 Hz, 1H), 8.02 (d, J = 5.2 Hz, 1H), 7.22 (d, J = 4.4 Hz, 1H), 5.27 (d, J = 5.6 Hz, 1H), 4.86-4.83 (m, 1H), 3.23-3.18 (m, 1H), 2.32-2.18 (m, 2H), 1.91-1.75 (m. 2H). |
| 66^{(a)(d)} | 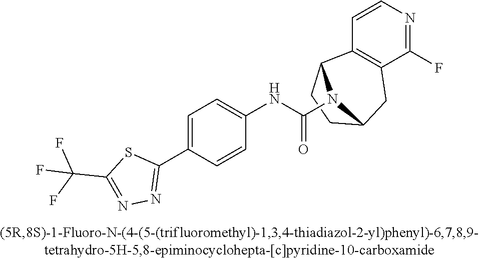 [SMILES: O=C(Nc1ccc(-c2nnc(C(F)(F)F)s2)cc1)N1[C@H]2CC[C@@H]1c1ccnc(F)c1C2] | Method 3 450.1 at 3.47 min | ¹H NMR (400 MHz, DMSO-d6) δ 9.16 (s, 1H), 8.02-7.96 (m, 3H), 7.73-7.70 (m, 2H), 7.22-7.21 (m, 1H), 5.28 (d, J = 6.0 Hz, 1H), 4.86 (t, J = 6.0 Hz, 1H), 3.20-3.14 (m, 1H), 2.61 (d, J = 17.6 Hz, 1H), 2.28-2.18 (m, 2H), 1.89-1.76 (m, 2H). |
| 67^{(b)(c)} | 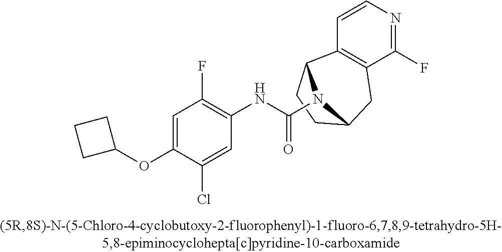 [SMILES: O=C(Nc1cc(Cl)c(OC2CCC2)cc1F)N1[C@H]2CC[C@@H]1c1ccnc(F)c1C2] | Method 1 420.2, 422.0 at 1.57 min | ¹H NMR (400 MHz, DMSO-d6) δ 8.50 (s, 1H), 7.99 (d, J = 5.0 Hz, 1H), 7.42 (d, J = 8.2 Hz, 1H), 7.17 (dd, J = 5.0, 1.7 Hz, 1H), 6.90 (d, J = 11.9 Hz, 1H), 5.15 (d, J = 6.0 Hz, 1H), 4.72 (td, J = 8.6, 5.0 Hz, 2H), 3.18 (dd, J = 17.5, 4.8 Hz, 1H), 2.55 (d, J = 17.2 Hz, 1H), 2.46-2.38 (m, 2H), 2.28-2.12 (m, 2H), 2.10-1.95 (m, 2H), 1.87-1.69 (m, 3H), 1.69-1.53 (m, 1H) |
| 68^{(b)(c)} | 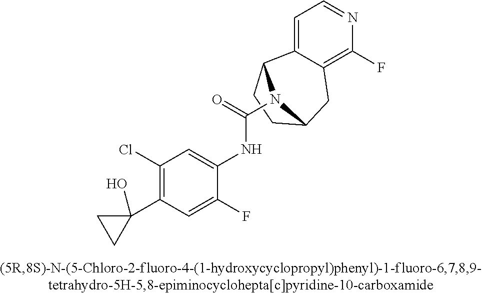 [SMILES: O=C(Nc1cc(Cl)c(C2(O)CC2)cc1F)N1[C@H]2CC[C@@H]1c1ccnc(F)c1C2] | Method 1 406.1, 408.3 at 1.21 min | ¹H NMR (400 MHz, DMSO-d6) δ 8.70 (s, 1H), 8.00 (d, J = 5.0 Hz, 1H), 7.55 (d, J = 7.3 Hz, 1H), 7.23 (d, J = 11.1 Hz, 1H), 7.19 (dd, J = 5.0, 1.7 Hz, 1H), 5.75 (s, 1H), 5.23-5.16 (m, 1H), 4.76 (t, J = 6.1 Hz, 1H), 3.18 (dd, J = 17.4, 5.0 Hz, 1H), 2.57 (d, J = 17.3 Hz, 1H), 2.31-2.12 (m, 2H), 1.88-1.70 (m, 2H), 1.00-0.92 (m, 2H), 0.89-0.78 (m, 2H). |
| 69^{(a)(d)} | 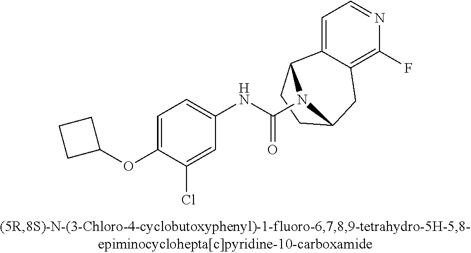 [SMILES: O=C(Nc1ccc(OC2CCC2)c(Cl)c1)N1[C@H]2CC[C@@H]1c1ccnc(F)c1C2] | Method 3 402.2 at 3.59 min | ¹H NMR (400 MHz, DMSO-d6) δ 8.69 (s, 1H), 7.99 (d, J = 4.8 Hz, 1H), 7.55 (d, J = 2.4 Hz, 1H), 7.28-7.25 (m, 1H), 7.18-7.17 (m, 1H), 6.85 (d, J = 9.2 Hz, 1H), 5.19-5.18 (m, 1H), 4.77-4.74 (m, 1H), 4.68-4.61 (m, 1H), 3.34-3.11 (m, 1H), 2.53-2.50 (m, 1H), 2.42-2.36 (m, 2H), 2.27-2.13 (m, 2H), 2.07-1.97 (m, 2H), 1.85-1.73 (m, 3H), 1.66-1.56 (m, 1H). |
| 70^{(a)(d)} | 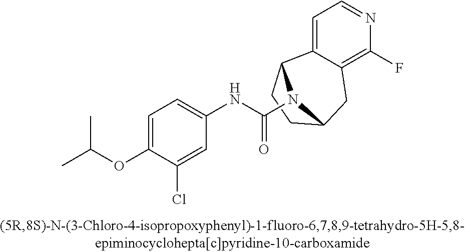 [SMILES: CC(C)Oc1ccc(NC(=O)N2[C@H]3CC[C@@H]2c2ccnc(F)c2C3)cc1Cl] | Method 3 390.2, 392.1 at 1.26 min | ¹H NMR (400 MHz, DMSO-d6) δ 8.69 (s, 1H), 8.00 (d, J = 4.8 Hz, 1H), 7.56 (d, J = 2.8 Hz, 1H), 7.29 (dd, J = 2.8, 9.2 Hz, 1H), 7.19 (d, J = 3.6 Hz, 1H), 7.04 (d, J = 8.8 Hz, 1H), 5.20 (d, J = 6 Hz, 1H), 4.78-4.75 (m, 1H), 4.52-4.46 (m, 1H), 3.30-3.11 (m, 1H), 2.58 (d, J = 17.6 Hz, 1H), 2.25-2.14 (m, 2H), 1.85-1.80 (m, 1H), 1.77-1.73 (m, 1H) |
| 71^{(a)(d)} | 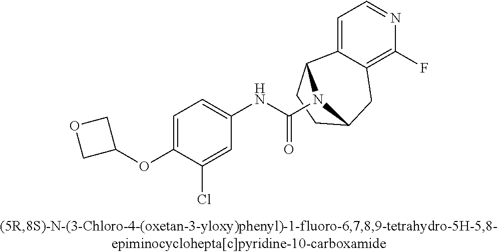 [SMILES: O=C(Nc1ccc(OC2COC2)c(Cl)c1)N1[C@H]2CC[C@@H]1c1ccnc(F)c1C2] | Method 4 403.1 at 3.63 min | ¹H NMR (400 MHz, DMSO-d6) δ 8.72 (s, 1H), 8.00 (d, J = 4.8 Hz, 1H), 6.43 (d, J = 2.4, Hz, 1H), 7.30 (dd, J = 2.4, 8.8 Hz, 1H), 7.19-7.17 (m, 1H), 6.69 (d, J = 8.8 Hz, 1H), 5.25-5.19 (m, 2H), 4.89 (t, J = 6.8 Hz, 2H). 4.78-4.74 (m, 2H), 4.55-4.52 (m, 2H), 3.15 (dd, J = 4.8, 17.6 Hz, 1H), 2.58 (d, J = 17.2 Hz, 1H), 2.27-2.12 (m, 2H), 1.85-1.71 (m, 2H) |
| 72^{(a)(d)} | 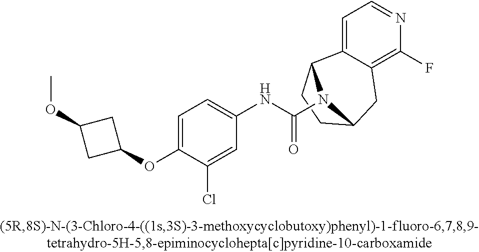 [SMILES: CO[C@H]1C[C@@H](Oc2ccc(NC(=O)N3[C@H]4CC[C@@H]3c3ccnc(F)c3C4)cc2Cl)C1] | Method 3 432.2 at 2.19 min | ¹H NMR (400 MHz, DMSO-d6) δ 8.70 (s, 1H), 8.00 (d, J = 4.8 Hz, 1H), 7.56 (d, J = 2.6 Hz, 1H), 7.29 (dd, J = 9.0, 2.6 Hz, 1H), 7.18 (dd, J = 5.0, 1.8 Hz, 1H), 6.88 (d, J = 9.0 Hz, 1H), 5.19 (d, J = 5.8 Hz, 1H), 4.76 (t, J = 6.0 Hz, 1H), 4.35 (p, J = 6.8 Hz, 1H), 3.60 (p, J = 7.0 Hz, 1H), 3.15-3.11 (m, 4H), 2.85-2.79 (m, 2H), 2.56 (d, J = 17.4 Hz, 1H), 2.31-2.10 (m, 2H), 1.93-1.69 (m, 4H). |
| 73^{(a)(d)} | 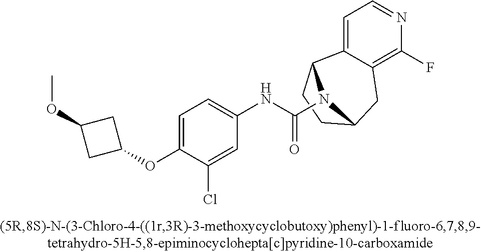 [SMILES: CO[C@H]1C[C@H](Oc2ccc(NC(=O)N3[C@H]4CC[C@@H]3c3ccnc(F)c3C4)cc2Cl)C1] | Method 3 432.2, 433.3 at 3.27 min | ¹H NMR (400 MHz, DMSO-d6) δ 8.73 (s, 1H), 8.02 (d, J = 5.0 Hz, 1H), 7.60 (d, J = 2.5 Hz, 1H), 7.31 (dd, J = 9.0, 2.6 Hz, 1H), 7.21 (dd, J = 5.1, 1.6 Hz, 1H), 6.85 (d, J = 9.0 Hz, 1H), 5.22 (d, J = 6.0 Hz, 1H), 4.88-4.73 (m, 2H), 4.07 (tt, J = 7.2, 4.1 Hz, 1H), 3.18 (s, 4H), 2.58 (d, J = 17.3 Hz, 1H), 2.48-2.35 (m, 2H), 2.33-2.13 (m, 4H), 1.85 (t, J = 10.0 Hz, 1H), 1.81-1.70 (m, 1H). |
| 77^{(b)(c)} | 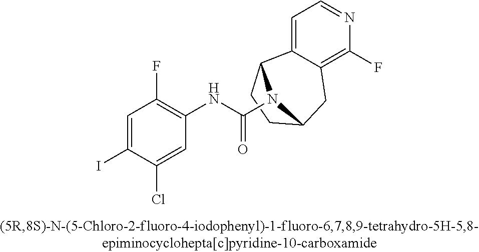 [SMILES: O=C(Nc1cc(Cl)c(I)cc1F)N1[C@H]2CC[C@@H]1c1ccnc(F)c1C2] | Method 1 475.8, 476.7 at 1.63 min | 1H NMR (400 MHz, DMSO-d6) δ 8.78 (s, 1H), 8.00 (d, J = 5.0 Hz, 1H), 7.80 (d, J = 10.0 Hz, 1H), 7.77 (d, J = 7.3 Hz, 1H), 7.19 (dd, J = 5.0, 1.7 Hz, 1H), 5.20 (d, J = 6.0 Hz, 1H), 4.77 (t, J = 6.0 Hz, 1H), 3.17 (dd, J = 17.4, 5.0 Hz, 1H), 2.57 (d, J = 17.3 Hz, 1H), 2.30-2.11 (m, 2H), 1.89-1.78 (m, 1H), 1.79-1.66 (m, 1H). |
| 78^{(b)(c)} | 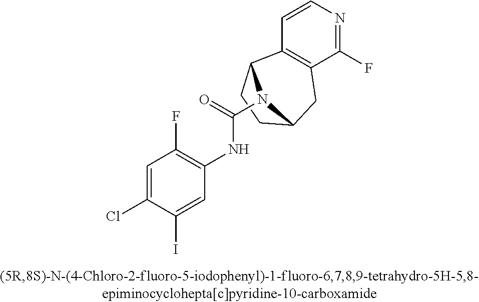 [SMILES: O=C(Nc1cc(I)c(Cl)cc1F)N1[C@H]2CC[C@@H]1c1ccnc(F)c1C2] | Method 1 476.1 at 1.64 min | ¹H NMR (400 MHz, DMSO-d6) δ 8.53 (s, 1H), 8.03 (d, J = 8.2 Hz, 1H), 8.00 (d, J = 5.0 Hz, 1H), 7.51 (d, J = 11.1 Hz, 1H), 7.21 (dd, J = 5.0, 1.7 Hz, 1H), 5.19 (d, J = 5.9 Hz, 1H), 4.78 (t, J = 6.1 Hz, 1H), 3.28 (d, J = 5.4 Hz, 1H), 2.60 (d, J = 17.3 Hz, 1H), 2.31-2.13 (m, 2H), 1.93-1.82 (m, 1H), 1.78 (dt, J = 9.6, 4.5 Hz, 1H). 1 proton partially under water signal |
| 79^{(b)(c)} | 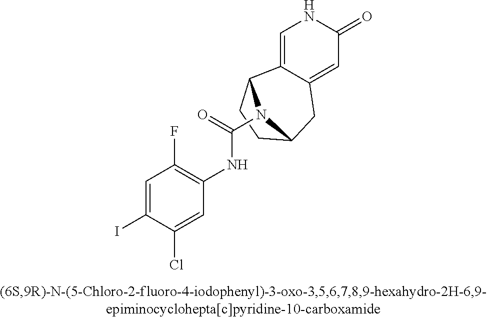 [SMILES: O=C(Nc1cc(Cl)c(I)cc1F)N1[C@H]2CC[C@@H]1c1c[nH]c(=O)cc1C2] | Method 1 474.1, 476.0 at 1.19 min | ¹H NMR (400 MHz, DMSO-d6) δ 11.26 (s, 1H), 8.58 (s, 1H), 7.81 (d, J = 7.4 Hz, 1H), 7.79 (d, J = 4.8 Hz, 1H), 7.18 (s, 1H), 6.09 (s, 1H), 5.03 (d, J = 5.8 Hz, 1H), 4.56 (t, J = 6.4 Hz, 1H), 3.11 (dd, J = 17.8, 5.0 Hz, 1H), 2.56 (s, 1H), 2.22-2.01 (m, 2H), 1.75-1.58 (m, 2H). |
| 80^{(b)(c)} | 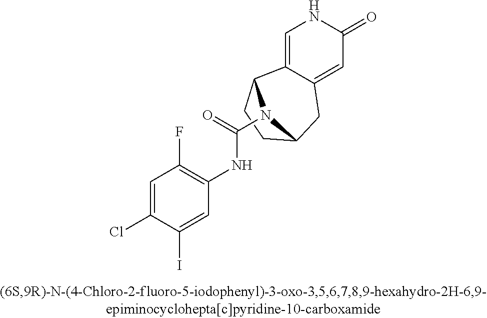 [SMILES: O=C(Nc1cc(I)c(Cl)cc1F)N1[C@H]2CC[C@@H]1c1c[nH]c(=O)cc1C2] | Method 1 474.1, 476.2 at 1.78 min | ¹H NMR (400 MHz, DMSO-d6) δ 11.24 (s, 1H), 8.30 (s, 1H), 8.04 (d, J = 8.2 Hz, 1H), 7.54 (d, J = 11.2 Hz, 1H), 7.21 (s, 1H), 6.10 (s, 1H), 5.02 (d, J = 5.9 Hz, 1H), 4.58 (t, J = 6.4 Hz, 1H), 3.28-3.14 (m, 1H), 2.57 (d, J = 17.8 Hz, 1H), 2.26-2.02 (m, 2H), 1.81-1.57 (m, 2H) |
| 81^{(a)(d)} | 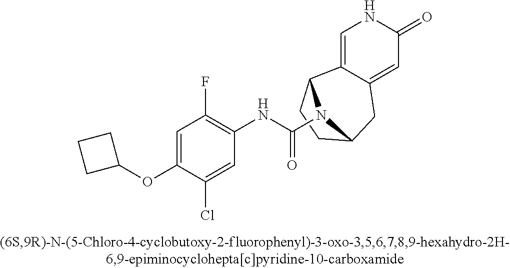 [SMILES: O=C(Nc1cc(Cl)c(OC2CCC2)cc1F)N1[C@H]2CC[C@@H]1c1c[nH]c(=O)cc1C2] | Method 3 418.1, 419.2 at 2.76 min | ¹H NMR (400 MHz, DMSO-d6) δ 11.27 (s, 1H), 8.33 (s, 1H), 7.45 (d, J = 8.2 Hz, 1H), 7.18 (s, 1H), 6.92 (d, J = 11.9 Hz, 1H), 6.12 (s, 1H), 5.00 (d, J = 5.9 Hz, 1H), 4.76 (p, J = 7.2 Hz, 1H), 4.55 (t, J = 6.4 Hz, 1H), 3.14 (dd, J = 17.8, 5.0 Hz, 1H), 2.56 (s, 1H), 2.46 (ddd, J = 11.9, 9.2, 4.4 Hz, 2H), 2.28-1.99 (m, 4H), 1.86-1.57 (m, 4H). |
| 82^{(b)(d)} | 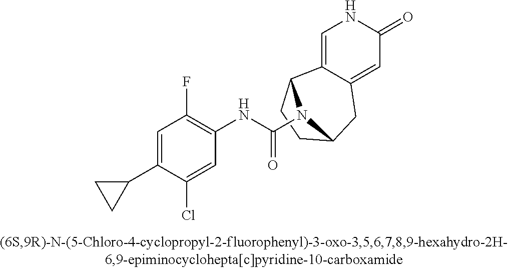 [SMILES: O=C(Nc1cc(Cl)c(C2CC2)cc1F)N1[C@H]2CC[C@@H]1c1c[nH]c(=O)cc1C2] | Method 1 388.1, 389.2 at 2.73 min | ¹H NMR (400 MHz, DMSO-d6) δ 11.28 (s, 1H), 8.46 (s, 1H), 7.57 (dd, J = 7.5, 4.1 Hz, 1H), 7.20 (s, 1H), 6.89 (d, J = 11.8 Hz, 1H), 6.12 (s, 1H), 5.04 (d, J = 6.0 Hz, 1H), 4.57 (t, J = 6.5 Hz, 1H), 3.21-3.02 (m, 1H), 2.57 (s, 1H), 2.23-1.96 (m, 3H), 1.70 (q, J = 11.9 Hz, 2H), 1.07-0.95 (m, 2H), 0.79-0.62 (m, 2H). |
| 84^{(b)(c)} | 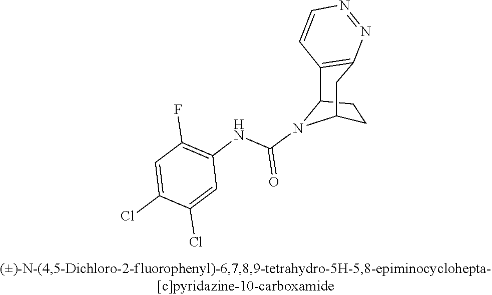 [SMILES: O=C(Nc1cc(Cl)c(Cl)cc1F)N1C2CCC1c1ccnnc1C2] | Method 1 367.1, 369.2 at 1.19 min | ¹H NMR (400 MHz, DMSO-d6) δ 9.00 (d, J = 5.1 Hz, 1H), 8.89 (s, 1H), 7.80 (d, J = 7.6 Hz, 1H), 7.67 (d, J = 10.3 Hz, 1H), 7.47 (d, J = 5.1 Hz, 1H), 5.21 (d, J = 6.1 Hz, 1H), 4.89-4.80 (m, 1H), 3.53 (dd, J = 17.6, 5.1 Hz, 1H), 3.00 (d, J = 17.7 Hz, 1H), 2.36-2.13 (m, 2H), 1.89-1.69 (m, 2H). |
| 85^{(b)(c)} | 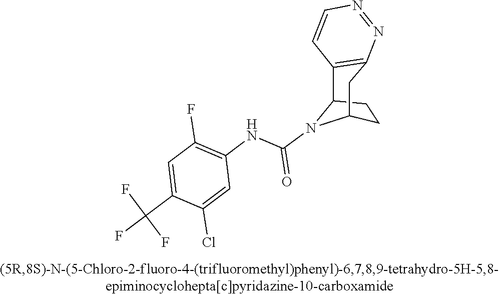 [SMILES: O=C(Nc1cc(Cl)c(C(F)(F)F)cc1F)N1C2CCC1c1ccnnc1C2] | Method 1 401.2, 403.2 at 1.32 min | ¹H NMR (400 MHz, DMSO-d6) δ 9.16 (s, 1H), 9.01 (d, J = 5.1 Hz, 1H), 8.00 (d, J = 6.9 Hz, 1H), 7.78 (d, J = 11.0 Hz, 1H), 7.49 (d, J = 5.1 Hz, 1H), 5.25 (d, J = 6.0 Hz, 1H), 4.95-4.85 (m, 1H), 3.54 (dd, J = 17.6, 5.1 Hz, 1H), 3.02 (d, J = 17.7 Hz, 1H), 2.35-2.13 (m, 2H), 1.91-1.72 (m, 2H). |
| 88^{(b)(c)} | 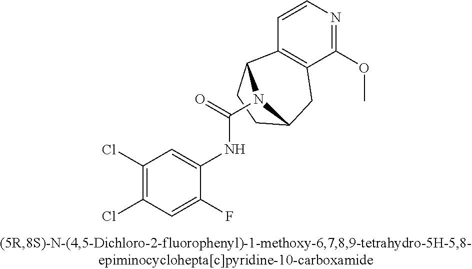 [SMILES: COc1nccc2c1C[C@@H]1CC[C@H]2N1C(=O)Nc1cc(Cl)c(Cl)cc1F] | Method 3 396.1, 398.1 at 6.67 min | ¹H NMR (400 MHz, DMSO-d6) δ 8.74 (s, 1H), 7.94 (d, J = 5.2 Hz, 1H), 7.81 (d, J = 7.6 Hz, 1H), 7.65 (d, J = 10.4 Hz, 1H), 6.81 (d, J = 4.8 Hz, 1H), 5.09 (d, J = 6 Hz, 1H), 4.75-4.72 (m, 1H), 3.84 (s, 3H), 3.08-3.02 (m, 1H), 2.41 (d, J = 17.6 Hz, 1H), 2.23-2.11 (m, 2H), 1.84-1.78 (m, 1H), 1.71-1.64 (m, 1H). |
| 89^{(a)(d)} | 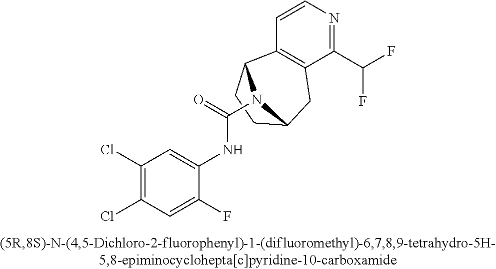 [SMILES: O=C(Nc1cc(Cl)c(Cl)cc1F)N1[C@H]2CC[C@@H]1c1ccnc(C(F)F)c1C2] | Method 4 416.2, 418.2 at 4.17 min | ¹H NMR (400 MHz, DMSO-d6) δ 8.83 (s, 1H), 8.43 (d, J = 5.2 Hz, 1H), 7.81 (d, J = 7.6 Hz, 1H), 7.66 (d, J = 10.4 Hz, 1H), 7.39 (d, J = 4.8 Hz, 1H), 6.95 (t, J = 67.6 Hz, 1H), 5.22 (d, J = 5.6 Hz, 1H), 4.77 (t, J = 6.0 Hz, 1H), 3.43 (d, J = 21.6 Hz, 1H), 2.84 (d, J = 17.6 Hz, 1H), 2.29-2.15 (m, 2H), 1.87-1.81 (m, 1H), 1.73-1.67 (m, 1H). |
| 90^{(a)(d)} | 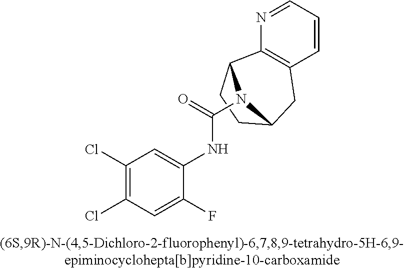 [SMILES: O=C(Nc1cc(Cl)c(Cl)cc1F)N1[C@H]2CC[C@@H]1c1ncccc1C2] | Method 3 366.2 at 2.79 min | ¹H NMR (400 MHz, DMSO-d6) δ 8.78 (s, 1H), 8.29-8.27 (m, 1H), 7.81 (d, J = 7.6 Hz, 1H), 7.64 (d, J = 10.4 Hz, 1H), 7.55-7.52 (m, 1H), 7.21-7.18 (m, 1H), 5.13 (d, J = 5.6 Hz, 1H), 4.70 (t, J = 5.6 Hz, 1H), 3.37 (d, J = 5.2 Hz, 1H), 2.25-2.14 (m, 2H), 2.03-1.97 (m, 1H), 1.85-1.80 (m, 1H), 1.68-1.62 (m, 1H). |
| 91^{(a)(d)} | 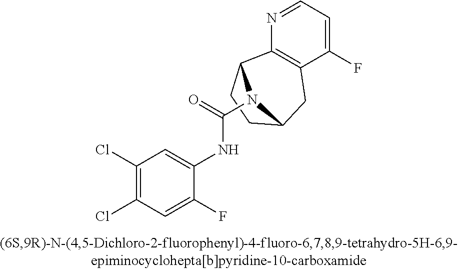 [SMILES: O=C(Nc1cc(Cl)c(Cl)cc1F)N1[C@H]2CC[C@@H]1c1nccc(F)c1C2] | Method 4 384.2 at 3.83 min | ¹H NMR (400 MHz, DMSO-d6) δ 8.94 (s, 1H), 8.40-8.36 (m, 1H), 7.81 (d, J = 7.2 Hz, 1H), 7.65 (d, J = 10.4 Hz, 1H), 7.15-7.11 (m, 1H), 5.42 (d, J = 6.4 Hz, 1H), 4.79-4.76 (m, 1H), 3.40-3.36 (m, 1H), 2.79-2.74 (m, 1H), 2.30-2.25 (m, 1H), 2.23-2.15 (m, 1H), 1.90-1.84 (m, 1H), 1.77-1.70 (m, 1H). |
| 92^{(a)(d)} | 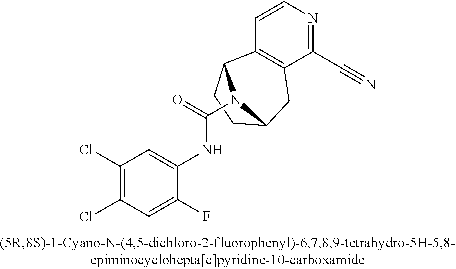 [SMILES: N#Cc1nccc2c1C[C@@H]1CC[C@H]2N1C(=O)Nc1cc(Cl)c(Cl)cc1F] | Method 3 391.1, 393.1 at 3.42 min | ¹H NMR (400 MHz, DMSO-d6) δ 8.84 (s, 1H), 8.51 (d, J = 4.8 Hz, 1H), 7.81 (d, J = 7.6 Hz, 1H), 7.66 (d, J = 10.4 Hz, 1H), 7.55 (d, J = 4.8 Hz, 1H), 5.23 (d, J = 6.0 Hz, 1H), 4.81 (t, J = 5.6 Hz, 1H), 3.44 (dd, J = 5.2, 17.2 Hz, 1H), 2.87 (d, J = 17.6 Hz, 1H), 2.25-2.18 (m, 2H), 1.82-1.78 (m, 2H). |
| 93^{(a)(d)} | 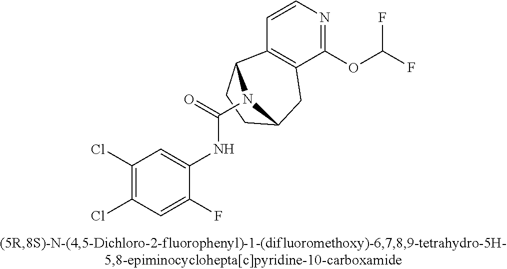 [SMILES: O=C(Nc1cc(Cl)c(Cl)cc1F)N1[C@H]2CC[C@@H]1c1ccnc(OC(F)F)c1C2] | Method 4 432.2 at 4.48 min | ¹H NMR (400 MHz, DMSO-d6) δ 8.81 (s, 1H), 8.05 (d, J = 5.1 Hz, 1H), 7.84 (d, J = 7.6 Hz, 1H), 7.69 (s, 1H), 7.67 (t, J = 63.3 Hz, 1H), 7.14 (d, J = 5.1 Hz, 1H), 5.19 (d, J = 5.8 Hz, 1H), 4.79 (t, J = 6.0 Hz, 1H), 3.14 (dd, J = 17.5, 5.0 Hz, 1H), 2.55 (s, 1H), 2.34-2.10 (m, 2H), 1.86 (dd, J = 11.3, 8.7 Hz, 1H), 1.80-1.69 (m, 1H). |
| 95^{(a)(d)} | 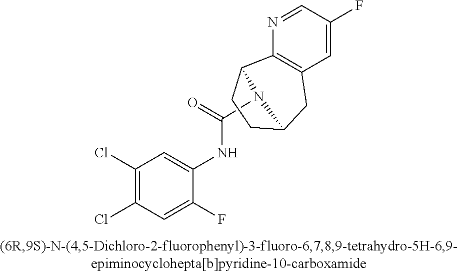 [SMILES: O=C(Nc1cc(Cl)c(Cl)cc1F)N1[C@@H]2CC[C@H]1c1ncc(F)cc1C2] | Method 3 384.1, 386.1 at 1.26 min | ¹H NMR (400 MHz, DMSO-d6) δ 8.81 (s, 1H), 8.28 (d, J = 2.8 Hz, 1H), 7.81 (d, J = 7.6 Hz, 1H), 7.66 (d, J = 10.4 Hz, 1H), 7.53 (dd, J = 9.6, 2.6 Hz, 1H), 5.16 (d, J = 5.6 Hz, 1H), 4.69 (t, J = 5.2 Hz, 1H), 3.36 (dd, J = 17.2, 4.8 Hz, 1H), 2.69 (d, J = 17.2 Hz, 1H), 2.27-2.13 (m, 2H), 1.83-1.78 (m, 1H), 1.68-1.62 (m, 1H). |
| 96^{(b)(e)} | 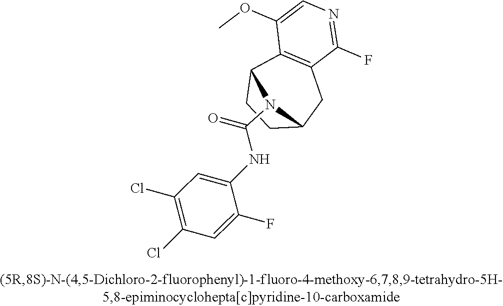 [SMILES: COc1cnc(F)c2c1[C@H]1CC[C@@H](C2)N1C(=O)Nc1cc(Cl)c(Cl)cc1F] | Method 4 414.2, 416.2 at 4.34 min | ¹H NMR (400 MHz, DMSO-d6) δ 8.86 (s, 1H), 7.82-7.80 (m, 1H), 7.72 (d, J = 1.6 Hz, 1H), 7.64 (d, J = 10 Hz, 1H), 5.34 (d, J = 6.4 Hz, 1H), 4.74 (t, J = 4.8 Hz, 1H), 3.89 (s, 3H), 3.19-3.13 (m, 2H), 2.30-2.22 (m, 2H), 2.18-2.10 (m, 1H), 1.81-1.68 (m, 1H). |
| 97^{(a)(d)} | 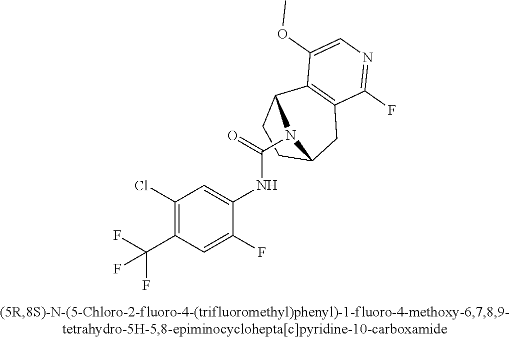 [SMILES: COc1cnc(F)c2c1[C@H]1CC[C@@H](C2)N1C(=O)Nc1cc(Cl)c(C(F)(F)F)cc1F] | Method 3 448.2, 450.2 at 3.86 min | ¹H NMR (400 MHz, DMSO-d6) δ 9.13 (br. s, 1H), 8.02 (d, J = 6.8 Hz, 1H), 7.78-7.73 (m, 2H), 5.39 (d, J = 6.4 Hz, 1H), 4.78 (t, J = 6.8 Hz, 1H), 3.90 (s, 3H), 3.20-3.15 (m, 1H), 2.57 (d, J = 17.4 Hz, 1H), 2.30-2.23 (m, 1H), 2.19-2.10 (m, 1H), 1.82-1.69 (m, 2H). |
| 98^{(a)(d)} | 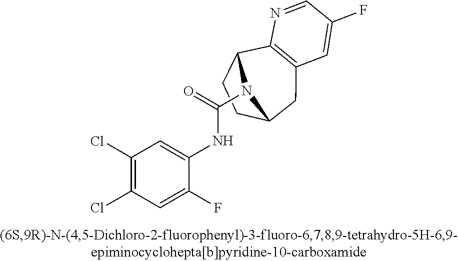 [SMILES: O=C(Nc1cc(Cl)c(Cl)cc1F)N1[C@H]2CC[C@@H]1c1ncc(F)cc1C2] | Method 3 384.1, 386.1 at 1.25 min | ¹H NMR (400 MHz, DMSO-d6) δ 8.81 (s, 1H), 8.28 (d, J = 2.4 Hz, 1H), 7.82 (d, J = 7.6 Hz, 1H), 7.66 (d, J = 10.4 Hz, 1H), 7.54-7.51 (m, 1H), 5.16 (d, J = 5.6 Hz, 1H), 4.69 (t, J = 5.2 Hz, 1H), 3.36 (dd, J = 17.2, 4.8 Hz, 1H), 2.69 (d, J = 17.2 Hz, 1H), 2.27-2.13 (m, 2H), 1.83-1.78 (m, 1H), 1.68-1.63 (m, 1H). |
| 102^{(a)(d)} | 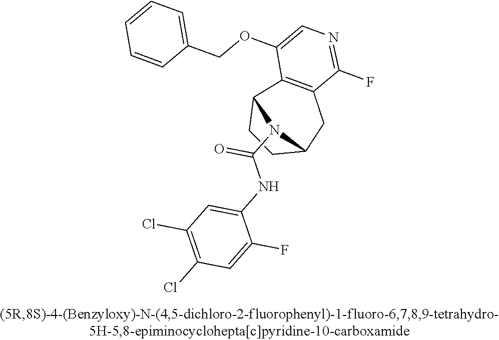 [SMILES: O=C(Nc1cc(Cl)c(Cl)cc1F)N1[C@H]2CC[C@@H]1c1c(OCc3ccccc3)cnc(F)c1C2] | Method 3 492.1 at 2.52 min | ¹H NMR (400 MHz, DMSO-d6) δ 8.88 (s, 1H), 7.86 (d, J = 7.6 Hz, 1H), 7.86 (s, 1H), 7.67 (d, J = 10.4 Hz, 1H), 7.49 (d, J = 8.4 Hz, 2H), 7.41-7.33 (m, 3H), 5.45 (d, J = 6.4 Hz, 1H), 5.25 (s, 2H), 4.75-4.72 (m, 1H), 3.21-3.15 (m, 1H), 2.56 (d, J = 24 Hz, 1H), 2.30-2.13 (m, 2H), 1.83-1.69 (m, 2H). |
| 103^{(a)(d)} | 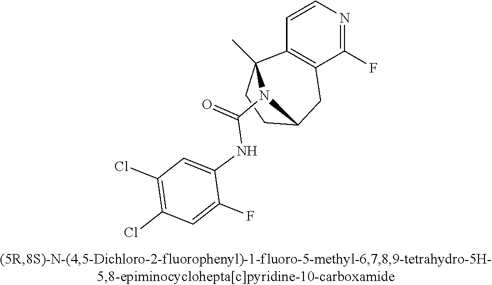 [SMILES: C[C@@]12CC[C@@H](Cc3c1ccnc3F)N2C(=O)Nc1cc(Cl)c(Cl)cc1F] | Method 3 398.1 at 3.74 min | ¹H NMR (400 MHz, DMSO-d6) δ 9.01 (s, 1H), 8.02 (d, J = 5.2 Hz, 1H), 7.76 (d, J = 7.6 Hz, 1H), 7.65 (d, J = 10.4 Hz, 1H), 7.22 (d, J = 6.0 Hz, 1H), 4.84 (t, J = 6.0 Hz, 1H), 3.17-3.11 (m, 1H), 2.59 (d, J = 17.6 Hz, 1H), 2.28-2.21 (m, 1H), 2.06-1.94 (m, 2H), 1.85 (s, 3H), 1.69-1.61 (m, 1H). |
| 106^{(a)(d)} | 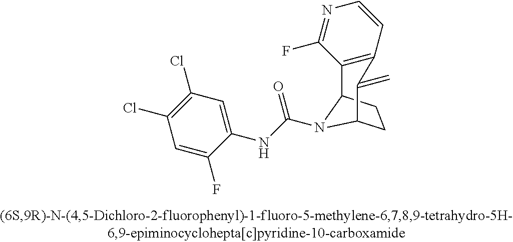 [SMILES: C=C1c2ccnc(F)c2C2CCC1N2C(=O)Nc1cc(Cl)c(Cl)cc1F] | Method 3 396.1, 398.1 at 3.66 min | ¹H NMR (400 MHz, DMSO-d6) δ 8.92 (s, 1H), 8.08 (d, J = 5.3 Hz, 1H), 7.78 (d, J = 7.6 Hz, 1H), 7.73 (dd, J = 5.5, 1.5 Hz, 1H), 7.67 (d, J = 10.3 Hz, 1H), 6.02 (s, 1H), 5.45 (s, 1H), 5.35 (d, J = 6.3 Hz, 1H), 5.19 (d, J = 7.7 Hz, 1H), 2.46 (td, J = 8.6, 4.2 Hz, 1H), 2.30 (tt, J = 12.0, 6.3 Hz, 1H), 1.91 (ddd, J = 12.4, 9.3, 3.1 Hz, 1H), 1.69 (ddd, J = 15.2, 9.2, 6.0 Hz, 1H). |
| 162^{(a)(c)} | 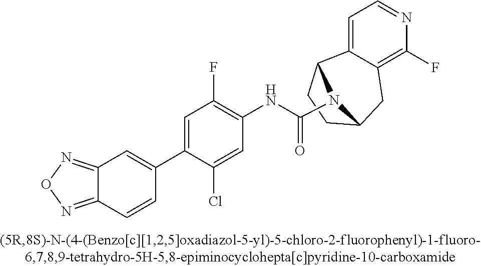 [SMILES: O=C(Nc1cc(Cl)c(-c2ccc3nonc3c2)cc1F)N1[C@H]2CC[C@@H]1c1ccnc(F)c1C2] | Method 2 468.3, 470.2 at 2.07 min | ¹H NMR (400 MHz, DMSO-d6) δ 8.92 (s, 1H), 8.15-8.13 (m, 1H), 8.13-8.10 (m, 1H), 8.02 (d, J = 5.0 Hz, 1H), 7.84 (d, J = 7.2 Hz, 1H), 7.68 (dd, J = 9.5, 1.2 Hz, 1H), 7.53 (d, J = 11.1 Hz, 1H), 7.22 (dd, J = 5.0, 1.6 Hz, 1H), 5.25 (d, J = 5.9 Hz, 1H), 4.82 (t, J = 6.1 Hz, 1H), 3.21 (dd, J = 17.5, 5.0 Hz, 1H), 2.60 (d, J = 17.4 Hz, 1H), 2.32-2.13 (m, 2H), 1.86 (t, J = 10.0 Hz, 1H), 1.78 (dd, J = 11.9, 5.6 Hz, 1H). |
| 167^{(a)(c)} | 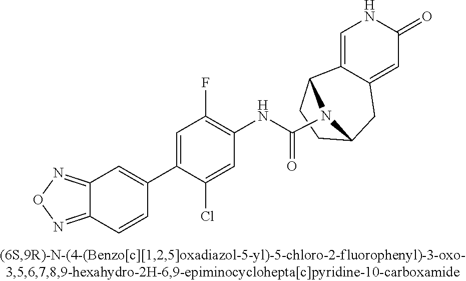 [SMILES: O=C(Nc1cc(Cl)c(-c2ccc3nonc3c2)cc1F)N1[C@H]2CC[C@@H]1c1c[nH]c(=O)cc1C2] | Method 2 466.2, 468.2 at 1.32 min | ¹H NMR (400 MHz, DMSO-d6) δ 11.28 (s, 1H), 8.72 (s, 1H), 8.20-8.08 (m, 2H), 7.87 (d, J = 7.2 Hz, 1H), 7.77-7.64 (m, 1H), 7.54 (d, J = 11.1 Hz, 1H), 7.21 (s, 1H), 6.11 (s, 1H), 5.08 (d, J = 5.8 Hz, 1H), 4.61 (t, J = 6.3 Hz, 1H), 3.16 (dd, J = 18.0, 5.1 Hz, 1H), 2.57 (d, J = 17.9 Hz, 1H), 2.26-2.03 (m, 2H), 1.77-1.60 (m, 2H). |
| 169^{(a)(c)} | 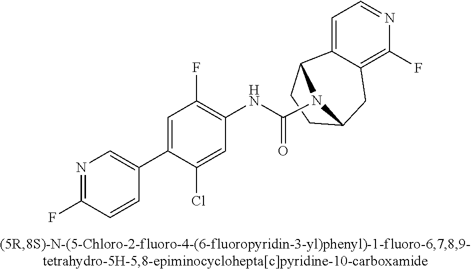 [SMILES: O=C(Nc1cc(Cl)c(-c2ccc(F)nc2)cc1F)N1[C@H]2CC[C@@H]1c1ccnc(F)c1C2] | Method 2 445.0, 447.0 at 1.94 min | ¹H NMR (400 MHz, DMSO-d6) δ 8.87 (s, 1H), 8.30 (d, J = 2.6 Hz, 1H), 8.09 (td, J = 8.2, 2.6 Hz, 1H), 8.01 (d, J = 5.0 Hz, 1H), 7.80 (d, J = 7.3 Hz, 1H), 7.44 (d, J = 11.1 Hz, 1H), 7.30 (dd, J = 8.6, 2.8 Hz, 1H), 7.21 (dd, J = 5.0, 1.6 Hz, 1H), 5.23 (d, J = 6.0 Hz, 1H), 4.81 (t, J = 6.1 Hz, 1H), 3.20 (dd, J = 17.4, 5.0 Hz, 1H), 2.59 (d, J = 17.4 Hz, 1H), 2.33-2.14 (m, 2H), 1.93-1.80 (m, 1H), 1.80-1.68 (m, 1H). |
| 172^{(a)(c)} | 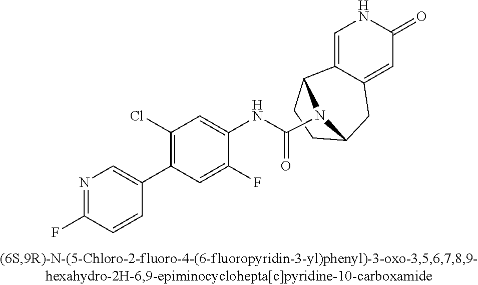 [SMILES: O=C(Nc1cc(Cl)c(-c2ccc(F)nc2)cc1F)N1[C@H]2CC[C@@H]1c1c[nH]c(=O)cc1C2] | Method 1 433.3, 435.2 at 1.15 min | ¹H NMR (400 MHz, DMSO-d6) δ 11.27 (s, 1H), 8.68 (s, 1H), 8.31 (d, J = 2.6 Hz, 1H), 8.10 (td, J = 8.2, 2.7 Hz, 1H), 7.82 (d, J = 7.3 Hz, 1H), 7.45 (d, J = 11.1 Hz, 1H), 7.30 (dd, J = 8.6, 2.8 Hz, 1H), 7.21 (s, 1H), 6.11 (s, 1H), 5.07 (d, J = 5.8 Hz, 1H), 4.61 (d, J = 7.1 Hz, 1H), 3.15 (dd, J = 16.3, 5.1 Hz, 1H), 2.56 (d, J = 17.8 Hz, 1H), 2.26-2.01 (m, 2H), 1.79-1.61 (m, 2H). |
| 176^{(a)(c)} | 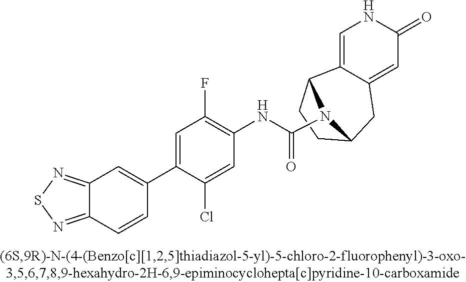 [SMILES: O=C(Nc1cc(Cl)c(-c2ccc3nsnc3c2)cc1F)N1[C@H]2CC[C@@H]1c1c[nH]c(=O)cc1C2] | Method 1 482.2, 484.2 at 1.30 min | ¹H NMR (400 MHz, DMSO-d6) δ 11.29 (s, 1H), 8.70 (s, 1H), 8.17 (d, J = 9.1 Hz, 2H), 7.90-7.77 (m, 2H), 7.53 (d, J = 11.1 Hz, 1H), 7.22 (s, 1H), 6.13 (s, 1H), 5.09 (d, J = 5.8 Hz, 1H), 4.63 (d, J = 7.0 Hz, 1H), 3.17 (d, J = 13.7 Hz, 1H), 2.62-2.52 (m, 1H), 2.24-2.04 (m, 2H) 1.74 (t, J = 10.2 Hz, 1H), 1.68 (s, 1H) |
| 177^{(a)(c)} | 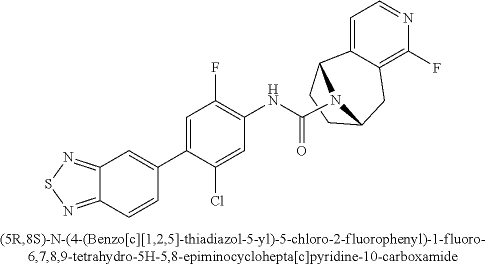 [SMILES: O=C(Nc1cc(Cl)c(-c2ccc3nsnc3c2)cc1F)N1[C@H]2CC[C@@H]1c1ccnc(F)c1C2] | Method 2 484.3, 486.3 at 2.07 min | ¹H NMR (400 MHz, DMSO-d6) δ 8.90 (s, 1H), 8.19-8.12 (m, 2H), 8.02 (d, J = 5.1 Hz, 1H), 7.83 (d, J = 7.3 Hz, 1H), 7.79 (dd, J = 9.0, 1.8 Hz, 1H), 7.52 (d, J = 11.1 Hz, 1H), 7.25-7.19 (m, 1H), 5.25 (d, J = 5.8 Hz, 1H), 4.84-4.80 (m, 1H), 3.26-3.18 (m, 1H), 2.61 (d, J = 17.3 Hz, 1H), 2.31-2.14 (m, 2H), 1.86 (t, J = 10.0 Hz, 1H), 1.79-1.75 (m, 1H). |
| 184^{(a)(c)} | 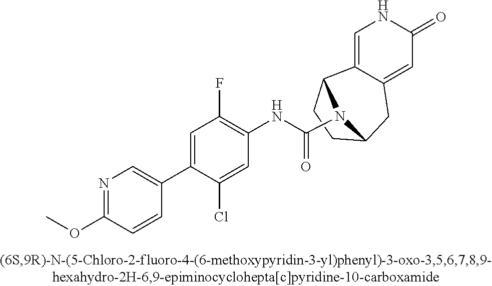 [SMILES: COc1ccc(-c2cc(F)c(NC(=O)N3[C@H]4CC[C@@H]3c3c[nH]c(=O)cc3C4)cc2Cl)cn1] | Method 1 455.2, 457.0 at 1.20 min | ¹H NMR (400 MHz, DMSO-d6) δ 11.27 (s, 1H), 8.63 (s, 1H), 8.23 (d, J = 2.6 Hz, 1H), 7.81 (dd, J = 8.6, 2.6 Hz, 1H), 7.77 (d, J = 7.4 Hz, 1H), 7.37 (d, J = 11.2 Hz, 1H), 7.20 (s, 1H), 6.91 (d, J = 8.4 Hz, 1H), 6.11 (s, 1H), 5.06 (d, J = 5.7 Hz, 1H), 4.60 (s, 1H), 3.90 (s, 3H), 3.14-3.09 (m, 1H), 2.56 (d, J = 18.1 Hz, 1H), 2.12-2.02 (m, 2H), 1.75-1.60 (m, 2H). |
| 185^{(a)(c)} | 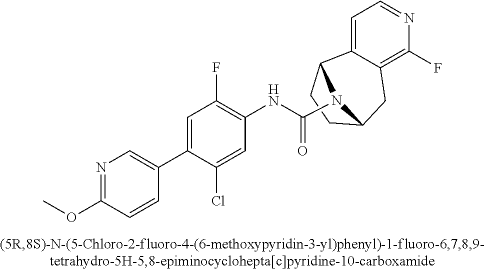 [SMILES: COc1ccc(-c2cc(F)c(NC(=O)N3[C@H]4CC[C@@H]3c3ccnc(F)c3C4)cc2Cl)cn1] | Method 1 457.2, 459.3 at 1.58 min | ¹H NMR (400 MHz, DMSO-d6) δ 8.83 (s, 1H), 8.22 (d, J = 2.5 Hz, 1H), 8.01 (d, J = 5.0 Hz, 1H), 7.80 (dd, J = 8.6, 2.5 Hz, 1H), 7.74 (d, J = 7.3 Hz, 1H), 7.36 (d, J = 11.1 Hz, 1H), 7.24-7.18 (m, 1H), 6.90 (d, J = 8.6 Hz, 1H), 5.23 (d, J = 5.9 Hz, 1H), 4.80 (s, 1H), 3.89 (s, 3H), 3.24-3.17 (m, 1H), 2.62-2.55 (m, 1H), 2.22 (dt, J = 11.8, 6.3 Hz, 2H), 1.89-1.82 (m, 1H), 1.79-1.72 (m, 1H). |
| 189^{(a)(c)} | 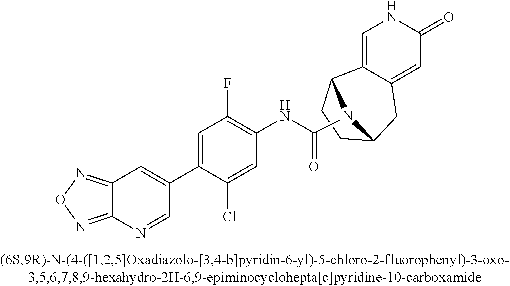 [SMILES: O=C(Nc1cc(Cl)c(-c2cnc3nonc3c2)cc1F)N1[C@H]2CC[C@@H]1c1c[nH]c(=O)cc1C2] | Method 1 467.3, 469.3 at 1.20 min | ¹H NMR (400 MHz, DMSO-d6) δ 11.29 (s, 1H), 9.19 (d, J = 2.1 Hz, 1H), 8.78 (s, 1H), 8.71 (d, J = 2.1 Hz, 1H), 7.93 (d, J = 7.2 Hz, 1H), 7.64 (d, J = 11.0 Hz, 1H), 7.22 (s, 1H), 6.12 (s, 1H), 5.09 (d, J = 5.8 Hz, 1H), 4.62 (t, J = 6.4 Hz, 1H), 3.21-3.11 (m, 1H), 2.60 (s, 1H), 2.26-2.04 (m, 2H), 1.71 (ddd, J = 22.9, 11.7, 7.3 Hz, 2H). |
| 190^{(a)(c)} | 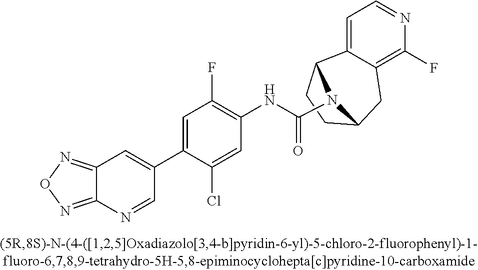 [SMILES: O=C(Nc1cc(Cl)c(-c2cnc3nonc3c2)cc1F)N1[C@H]2CC[C@@H]1c1ccnc(F)c1C2] | Method 2 468.9, 471.6 at 1.57 min | ¹H NMR (400 MHz, DMSO-d6) δ 9.18 (d, J = 2.1 Hz, 1H), 8.97 (s, 1H), 8.71 (d, J = 2.1 Hz, 1H), 8.02 (d, J = 5.0 Hz, 1H), 7.90 (d, J = 7.2 Hz, 1H), 7.63 (d, J = 11.0 Hz, 1H), 7.22 (dd, J = 5.0, 1.6 Hz, 1H), 5.25 (d, J = 5.9 Hz, 1H), 4.83 (t, J = 6.2 Hz, 1H), 3.22 (dd, J = 17.5, 4.9 Hz, 1H), 2.61 (d, J = 17.3 Hz, 1H), 2.22 (ddd, J = 17.8, 12.0, 6.4 Hz, 2H), 1.93-1.73 (m, 2H). |
| 194^{(a)(c)} | 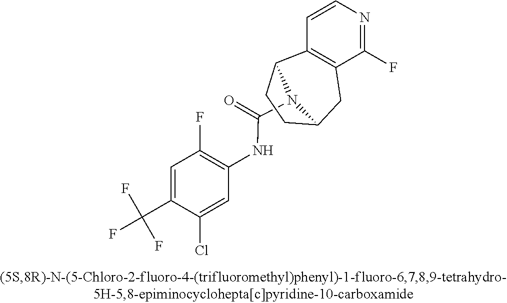 [SMILES: O=C(Nc1cc(Cl)c(C(F)(F)F)cc1F)N1[C@@H]2CC[C@H]1c1ccnc(F)c1C2] | Method 3 3418.1 at 3.53 min | ¹H NMR (400 MHz, DMSO-d6) δ 9.07 (s, 1H), 8.02-8.00 (m, 2H), 7.77 (d, J = 11.2 Hz, 1H), 7.21-7.19 (m, 1H), 5.24 (d, J = 6.0 Hz, 1H), 4.81 (t, J = 6.4 Hz, 1H), 3.18 (dd, J = 17.6, 4.8 Hz, 1H), 2.61-2.57 (m, 1H), 2.29-2.15 (m, 2H), 1.87-1.72 (m, 2H). |
| 200^{(a)(c)} | 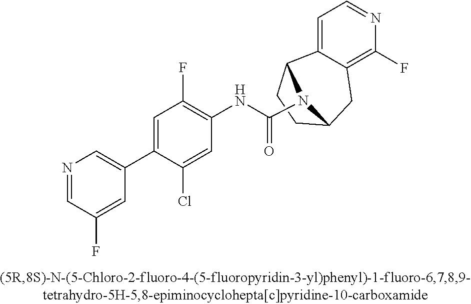 [SMILES: O=C(Nc1cc(Cl)c(-c2cncc(F)c2)cc1F)N1[C@H]2CC[C@@H]1c1ccnc(F)c1C2] | Method 2 445.2, 447.2 at 1.81 min | ¹H NMR (400 MHz, DMSO-d6) δ 8.89 (s, 1H), 8.63 (d, J = 2.8 Hz, 1H), 8.53 (t, J = 1.8 Hz, 1H), 8.01 (d, J = 5.0 Hz, 1H), 7.90 (ddd, J = 9.9, 2.8, 1.8 Hz, 1H), 7.82 (d, J = 7.2 Hz, 1H), 7.48 (d, J = 11.1 Hz, 1H), 7.21 (d, J = 4.8 Hz, 1H), 5.24 (d, J = 5.9 Hz, 1H), 4.83-4.79 (m, 1H), 3.25-3.13 (m, 1H), 2.60 (d, J = 17.4 Hz, 1H), 2.30-2.17 (m, 2H), 1.90-1.74 (m, 2H). |
| 201^{(a)(c)} | 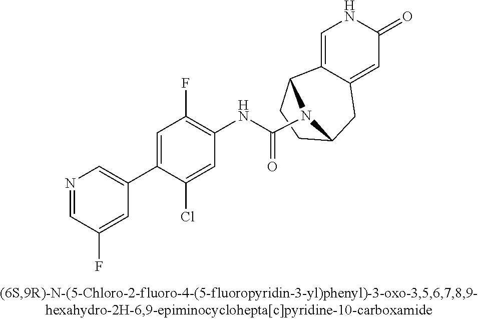 [SMILES: O=C(Nc1cc(Cl)c(-c2cncc(F)c2)cc1F)N1[C@H]2CC[C@@H]1c1c[nH]c(=O)cc1C2] | Method 2 443.3, 445.2 at 1.36 min | ¹H NMR (400 MHz, DMSO-d6) δ 11.28 (s, 1H), 8.69 (s, 1H), 8.63 (d, J = 2.7 Hz, 1H), 8.54 (t, J = 1.8 Hz, 1H), 7.90 (ddd, J = 9.9, 2.8, 1.8 Hz, 1H), 7.84 (d, J = 7.2 Hz, 1H), 7.49 (d, J = 11.1 Hz, 1H), 7.21 (s, 1H), 6.11 (s, 1H), 5.07 (d, J = 5.9 Hz, 1H), 4.61 (t, J = 6.3 Hz, 1H), 3.20-3.10 (m, 1H), 2.56 (d, J = 18.0 Hz, 1H), 2.25-2.02 (m, 2H), 1.77-1.60 (m, 2H). |
| 205^{(a)(c)} | 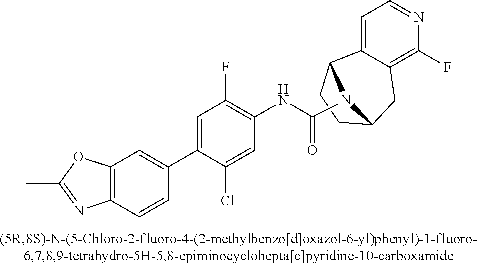 [SMILES: Cc1nc2ccc(-c3cc(F)c(NC(=O)N4[C@H]5CC[C@@H]4c4ccnc(F)c4C5)cc3Cl)cc2o1] | Method 2 481.2, 483.3 at 1.95 min | ¹H NMR (500 MHz, DMSO-d6) δ 8.83 (s, 1H), 8.01 (d, J = 5.0 Hz, 1H), 7.78-7.68 (m, 3H), 7.40-7.33 (m, 2H), 7.21 (d, J = 5.0 Hz, 1H), 5.26-5.21 (m, 1H), 4.88-4.76 (m, 1H), 3.21 (dd, J = 16.9, 5.1 Hz, 1H), 2.65-2.55 (m, 4H), 2.29-2.18 (m, 2H), 1.89-1.82 (m, 1H), 1.81-1.72 (m, 1H). |
| 206^{(a)(c)} | 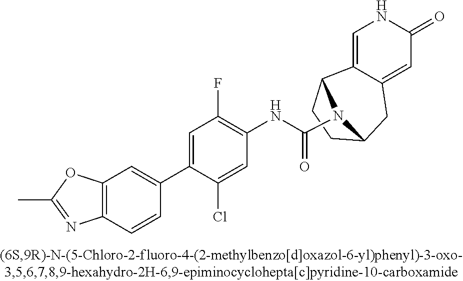 [SMILES: Cc1nc2ccc(-c3cc(F)c(NC(=O)N4[C@H]5CC[C@@H]4c4c[nH]c(=O)cc4C5)cc3Cl)cc2o1] | Method 2 479.2, 481.3 at 1.55 min | ¹H NMR (500 MHz, DMSO-d6) δ 11.27 (s, 1H), 8.64 (s, 1H), 7.78 (d, J = 7.3 Hz, 1H), 7.75-7.73 (m, 1H), 7.71 (d, J = 8.2 Hz, 1H), 7.41-7.34 (m, 2H), 7.20 (s, 1H), 6.11 (s, 1H), 5.07 (d, J = 6.1 Hz, 1H), 4.60 (t, J = 7.7 Hz, 1H), 3.20-3.11 (m, 1H), 2.64 (s, 3H), 2.56 (d, J = 17.8 Hz, 1H), 2.22-2.04 (m, 2H), 1.79-1.61 (m, 2H). |
| 214^{(a)(e)} | 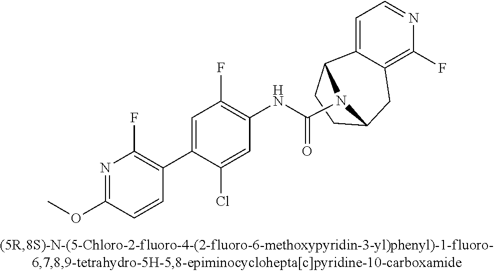 [SMILES: COc1ccc(-c2cc(F)c(NC(=O)N3[C@H]4CC[C@@H]3c3ccnc(F)c3C4)cc2Cl)c(F)n1] | Method 1 475.2 at 1.70 min | ¹H NMR (400 MHz, DMSO-d6) δ 8.87 (s, 1H), 8.01 (d, J = 4.9 Hz, 1H), 7.86 (dd, J = 10.0, 8.2 Hz, 1H), 7.76 (d, J = 7.3 Hz, 1H), 7.39 (d, J = 11.0 Hz, 1H), 7.23-7.19 (m, 1H), 6.88 (dd, J = 8.1, 1.2 Hz, 1H), 5.23 (d, J = 5.9 Hz, 1H), 4.80 (s, 1H), 3.88 (s, 3H), 3.24-3.18 (m, 1H), 2.59 (d, J = 17.4 Hz, 1H), 2.30-2.16 (m, 2H), 1.85 (t, J = 10.0 Hz, 1H), 1.77 (d, J = 7.5 Hz, 1H). |
| 217^{(a)(e)} | 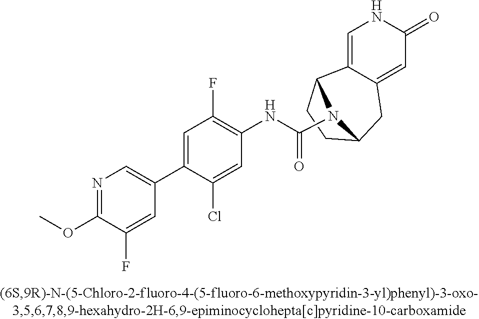 [SMILES: COc1ncc(-c2cc(F)c(NC(=O)N3[C@H]4CC[C@@H]3c3c[nH]c(=O)cc3C4)cc2Cl)cc1F] | Method 1 473.2, 475.1 at 1.25 min | ¹H NMR (400 MHz, DMSO-d6) δ 11.27 (bs, 1H), 8.64 (s, 1H), 8.23 (d, J = 3.0 Hz, 1H), 7.78-7.69 (m, 2H), 7.34 (d, J = 10.9 Hz, 1H), 7.20 (s, 1H), 6.11 (s, 1H), 5.06 (d, J = 5.8 Hz, 1H), 4.59 (t, J = 6.3 Hz, 1H), 3.82 (s, 3H), 3.15 (dd, J = 17.9, 5.1 Hz, 1H), 2.56 (d, J = 17.9 Hz, 1H), 2.25-2.02 (m, 2H), 1.77-1.60 (m, 2H). |
| 219^{(a)(e)} | 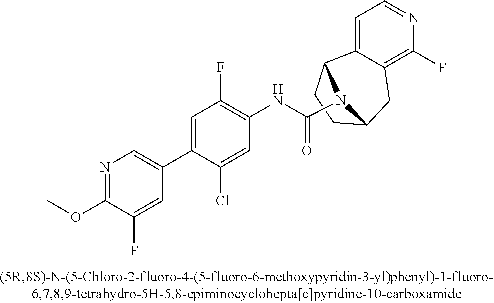 [SMILES: COc1ncc(-c2cc(F)c(NC(=O)N3[C@H]4CC[C@@H]3c3ccnc(F)c3C4)cc2Cl)cc1F] | Method 1 475.2, 477.1 at 1.58 min | ¹H NMR (400 MHz, DMSO-d6) δ 8.84 (s, 1H), 8.23 (d, J = 3.0 Hz, 1H), 8.01 (d, J = 5.0 Hz, 1H), 7.76-7.68 (m, 2H), 7.34 (d, J = 10.9 Hz, 1H), 7.21 (dd, J = 5.0, 1.6 Hz, 1H), 5.22 (d, J = 5.9 Hz, 1H), 4.80 (t, J = 6.1 Hz, 1H), 3.81 (s, 3H), 3.21 (dd, J = 17.5, 5.0 Hz, 1H), 2.59 (d, J = 17.4 Hz, 1H), 2.32-2.14 (m, 2H), 1.90-1.71 (m, 2H). |
| 220^{(a)(e)} | 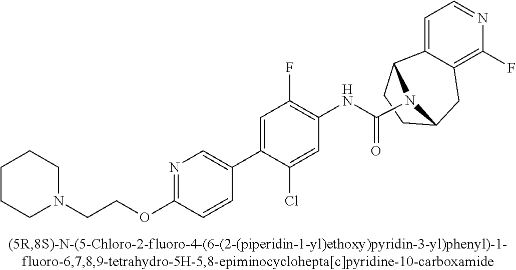 [SMILES: O=C(Nc1cc(Cl)c(-c2ccc(OCCN3CCCCC3)nc2)cc1F)N1[C@H]2CC[C@@H]1c1ccnc(F)c1C2] | Method 1 554.3, 556.4 at 1.81 min | ¹H NMR (400 MHz, DMSO-d6) δ 8.86 (s, 1H), 8.23 (dd, J = 2.5, 0.8 Hz, 1H), 8.13 (s, 1H), 8.03-7.99 (m, 1H), 7.84 (dd, J = 8.6, 2.5 Hz, 1H), 7.75 (d, J = 7.4 Hz, 1H), 7.37 (d, J = 11.0 Hz, 1H), 7.21 (dd, J = 5.1, 1.7 Hz, 1H), 6.94 (d, J = 8.6 Hz, 1H), 5.25-5.19 (m, 1H), 4.83-4.76 (m, 1H), 4.58-4.49 (m, 2H), 3.23-3.17 (m, 2H), 3.05-2.71 (m, 4H), 2.63-2.56 (m, 1H), 2.29-2.15 (m, 2H), 1.89-1.81 (m, 1H), 1.81-1.72 (m, 1H), 1.69-1.59 (m, 4H), 1.52-1.41 (m, 2H). |
| 222^{(a)(e)} | 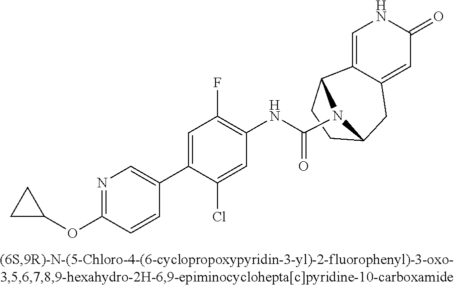 [SMILES: O=C(Nc1cc(Cl)c(-c2ccc(OC3CC3)nc2)cc1F)N1[C@H]2CC[C@@H]1c1c[nH]c(=O)cc1C2] | Method 1 481.3, 483.3 at 1.29 min | ¹H NMR (400 MHz, DMSO-d6) δ 11.30 (s, 1H), 8.65 (s, 1H), 8.25 (d, J = 2.5 Hz, 1H), 8.18 (s, 1H), 7.83 (dd, J = 8.6, 2.6 Hz, 1H), 7.77 (d, J = 7.4 Hz, 1H), 7.39 (d, J = 11.1 Hz, 1H), 7.21 (s, 1H), 6.95 (d, J = 8.8 Hz, 1H), 6.11 (s, 1H), 5.06 (d, J = 5.9 Hz, 1H), 4.62-4.56 (m, 1H), 4.24 (tt, J = 6.2, 3.1 Hz, 1H), 3.17-3.11 (m, 1H), 2.62-2.52 (m, 1H), 2.26-2.12 (m, 1H), 1.75-1.61 (m, 2H), 0.83-0.74 (m, 2H), 0.74-0.67 (m, 2H). |
| 223^{(a)(e)} | 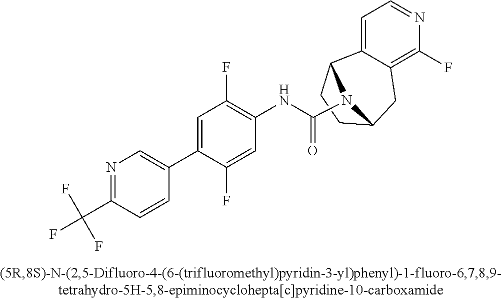 [SMILES: O=C(Nc1cc(F)c(-c2ccc(C(F)(F)F)nc2)cc1F)N1[C@H]2CC[C@@H]1c1ccnc(F)c1C2] | Method 1 478.7 at 1.63 min | ¹H NMR (400 MHz, DMSO-d6) δ 8.94 (d, J = 8.9 Hz, 2H), 8.27 (d, J = 8.2 Hz, 1H), 8.04-7.97 (m, 2H), 7.72-7.62 (m, 2H), 7.22 (dd, J = 5.0, 1.6 Hz, 1H), 5.26 (d, J = 5.9 Hz, 1H), 4.83 (t, J = 6.1 Hz, 1H), 3.20 (dd, J = 17.5, 5.0 Hz, 1H), 2.60 (d, J = 17.3 Hz, 1H), 2.33-2.14 (m, 2H), 1.93-1.71 (m, 2H). |
| 232^{(a)(e)} | 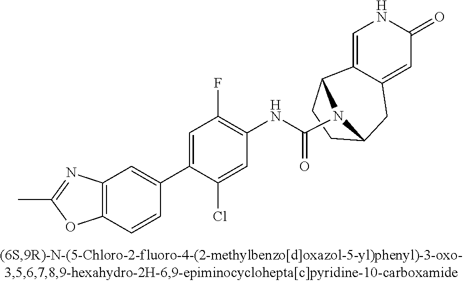 [SMILES: Cc1nc2cc(-c3cc(F)c(NC(=O)N4[C@H]5CC[C@@H]4c4c[nH]c(=O)cc4C5)cc3Cl)ccc2o1] | Method 1 479.1, 481.1 at 1.23 min | ¹H NMR (400 MHz, DMSO-d6) δ 11.28 (s, 1H), 8.63 (s, 1H), 7.80-7.67 (m, 3H), 7.42-7.32 (m, 2H), 7.21 (s, 1H), 6.11 (s, 1H), 5.07 (d, J = 5.9 Hz, 1H), 4.60 (t, J = 6.4 Hz, 1H), 3.16 (dd, J = 18.0, 5.1 Hz, 1H), 2.64 (s, 3H), 2.56 (d, J = 17.8 Hz, 1H), 2.25-2.04 (m, 2H), 1.77-1.60 (m, 2H). |
| 233^{(a)(e)} | 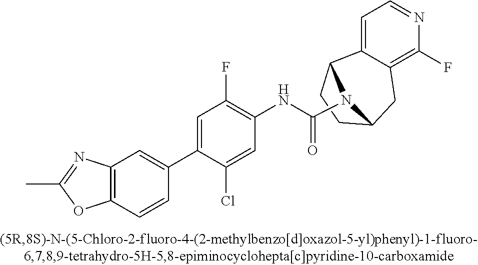 [SMILES: Cc1nc2cc(-c3cc(F)c(NC(=O)N4[C@H]5CC[C@@H]4c4ccnc(F)c4C5)cc3Cl)ccc2o1] | Method 1 481.1, 483.1 at 1.50 min | ¹H NMR (400 MHz, DMSO-d6) δ 8.83 (s, 1H), 8.01 (d, J = 5.0 Hz, 1H), 7.78-7.64 (m, 3H), 7.41-7.32 (m, 2H), 7.21 (dd, J = 5.0, 1.7 Hz, 1H), 5.23 (d, J = 5.9 Hz, 1H), 4.84-4.77 (m, 1H), 3.21 (dd, J = 17.4, 5.0 Hz, 1H), 2.66-2.55 (m, 4H), 2.31-2.16 (m, 3H), 1.90-1.73 (m, 2H). |
| 239^{(a)(e)} | 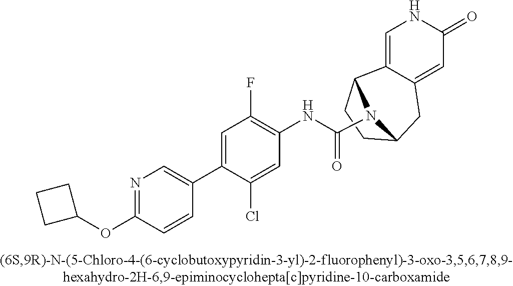 [SMILES: O=C(Nc1cc(Cl)c(-c2ccc(OC3CCC3)nc2)cc1F)N1[C@H]2CC[C@@H]1c1c[nH]c(=O)cc1C2] | Method 1 495.3, 497.3 at 1.50 min | ¹H NMR (400 MHz, DMSO-d6) δ 11.29 (s, 1H), 8.64 (s, 1H), 8.18 (dd, J = 2.5, 0.8 Hz, 1H), 7.79 (dd, J = 8.6, 2.6 Hz, 1H), 7.76 (d, J = 7.4 Hz, 1H), 7.37 (d, J = 11.1 Hz, 1H), 7.20 (s, 1H), 6.85 (dd, J = 8.6, 0.7 Hz, 1H), 6.11 (s, 1H), 5.16 (p, J = 7.4 Hz, 1H), 5.05 (d, J = 5.8 Hz, 1H), 4.62-4.55 (m, 1H), 3.14 (dd, J = 17.8, 5.0 Hz, 1H), 2.55 (d, J = 17.7 Hz, 1H), 2.46-2.36 (m, 2H), 2.23-2.13 (m, 1H), 2.13-2.01 (m, 3H), 1.84-1.60 (m, 4H). |
| 240^{(a)(e)} | 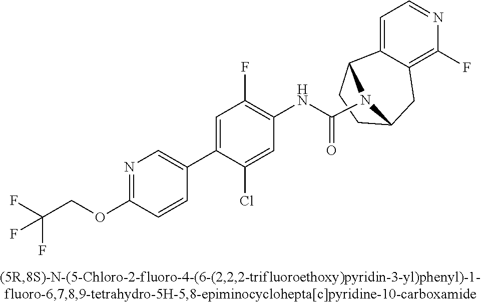 [SMILES: O=C(Nc1cc(Cl)c(-c2ccc(OCC(F)(F)F)nc2)cc1F)N1[C@H]2CC[C@@H]1c1ccnc(F)c1C2] | Method 2 525.3, 527.2 at 2.30 min | ¹H NMR (400 MHz, DMSO-d6) δ 8.86 (s, 1H), 8.28-8.24 (m, 1H), 8.01 (d, J = 5.0 Hz, 1H), 7.91 (dd, J = 8.6, 2.5 Hz, 1H), 7.76 (d, J = 7.3 Hz, 1H), 7.40 (d, J = 11.0 Hz, 1H), 7.21 (dd, J = 5.1, 1.6 Hz, 1H), 7.08 (d, J = 8.8 Hz, 1H), 5.23 (d, J = 5.9 Hz, 1H), 5.04 (q, J = 9.1 Hz, 2H), 4.80 (t, J = 6.1 Hz, 1H), 3.20 (dd, J = 17.4, 5.0 Hz, 1H), 2.59 (d, J = 17.3 Hz, 1H), 2.29-2.15 (m, 2H), 1.89-1.81 (m, 1H), 1.80-1.72 (m, 1H). |
| 241^{(a)(e)} | 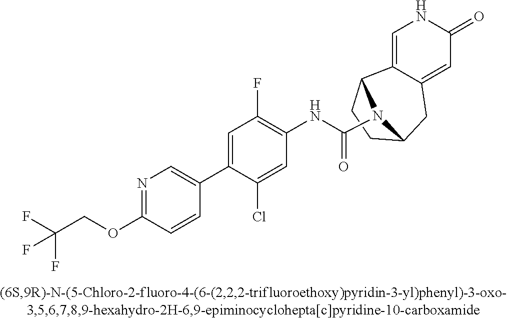 [SMILES: O=C(Nc1cc(Cl)c(-c2ccc(OCC(F)(F)F)nc2)cc1F)N1[C@H]2CC[C@@H]1c1c[nH]c(=O)cc1C2] | Method 1 523.3, 525.3 at 1.48 min | ¹H NMR (400 MHz, DMSO-d6) δ 11.29 (s, 1H), 8.67 (s, 1H), 8.27 (dd, J = 2.5, 0.8 Hz, 1H), 7.91 (dd, J = 8.6, 2.5 Hz, 1H), 7.79 (d, J = 7.3 Hz, 1H), 7.41 (d, J = 11.0 Hz, 1H), 7.21 (s, 1H), 7.15-7.03 (m, 1H), 6.11 (s, 1H), 5.14-4.93 (m, 3H), 4.60 (t, J = 6.3 Hz, 1H), 3.14 (dd, J = 17.9, 5.0 Hz, 1H), 2.56 (d, J = 18.0 Hz, 1H), 2.29-1.99 (m, 2H), 1.82-1.54 (m, 2H). |
| 242^{(a)(e)} | 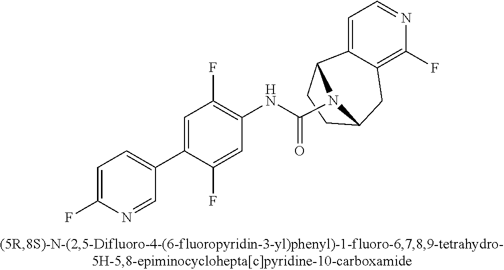 [SMILES: O=C(Nc1cc(F)c(-c2ccc(F)nc2)cc1F)N1[C@H]2CC[C@@H]1c1ccnc(F)c1C2] | Method 2 428.8 at 1.92 min | ¹H NMR (400 MHz, DMSO-d6) δ 8.94 (d, J = 8.9 Hz, 2H), 8.27 (d, J = 8.2 Hz, 1H), 8.04-7.97 (m, 2H), 7.72-7.62 (m, 2H), 7.22 (dd, J = 5.0, 1.6 Hz, 1H), 5.26 (d, J = 5.9 Hz, 1H), 4.83 (t, J = 6.1 Hz, 1H), 3.20 (dd, J = 17.5, 5.0 Hz, 1H), 2.60 (d, J = 17.3 Hz, 1H), 2.33-2.14 (m, 2H), 1.93-1.71 (m, 2H). |
| 243^{(a)(e)} | 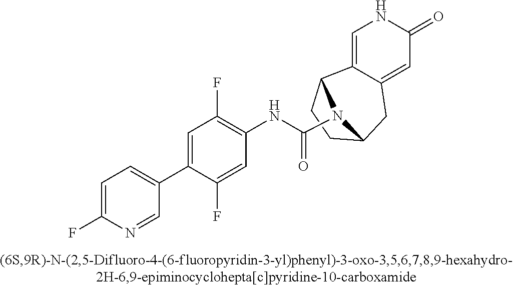 [SMILES: O=C(Nc1cc(F)c(-c2ccc(F)nc2)cc1F)N1[C@H]2CC[C@@H]1c1c[nH]c(=O)cc1C2] | Method 1 427.3 at 1.52 min | ¹H NMR (400 MHz, DMSO-d6) δ 11.2 (s, 1H), 8.66 (s, 1H), 8.43 (d, J = 2.5 Hz, 1H), 8.23-8.13 (m, 1H), 7.64 (dd, J = 12.5, 6.7 Hz, 1H), 7.56 (dd, J = 11.3, 7.2 Hz, 1H), 7.31 (dd, J = 8.6, 2.9 Hz, 1H), 7.21 (s, 1H), 6.11 (s, 1H), 5.08 (d, J = 5.8 Hz, 1H), 4.61 (t, J = 6.2 Hz, 1H), 3.14 (dd, J = 18.0, 4.9 Hz, 1H), 2.56 (d, J = 17.8 Hz, 1H), 2.23-2.09 (m, 2H), 1.77-1.60 (m, 2H). |
| 245^{(a)(e)} | 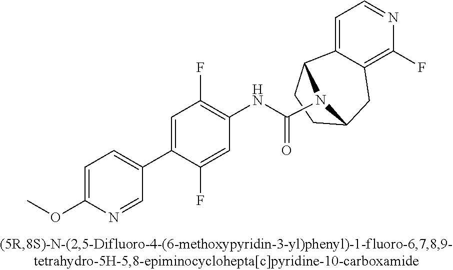 [SMILES: COc1ccc(-c2cc(F)c(NC(=O)N3[C@H]4CC[C@@H]3c3ccnc(F)c3C4)cc2F)cn1] | Method 1 441.3 at 1.54 min | ¹H NMR (400 MHz, DMSO-d6) δ 8.81 (s, 1H), 8.34 (s, 1H), 8.01 (d, J = 4.5 Hz, 1H), 7.93-7.85 (m, 1H), 7.55 (dd, J = 12.3, 6.8 Hz, 1H), 7.47 (dd, J = 11.3, 7.3 Hz, 1H), 7.21 (dd, J = 5.0, 1.6 Hz, 1H), 6.91 (d, J = 9.0 Hz, 1H), 5.24 (d, J = 5.9 Hz, 1H), 4.82 (d, J = 6.1 Hz, 1H), 3.89 (s, 3H), 3.20 (dd, J = 17.3, 5.0 Hz, 1H), 2.59 (d, J = 17.3 Hz, 1H), 2.30-2.16 (m, 2H), 1.85 (t, J = 9.8 Hz, 1H), 1.76 (s, 1H). |
| 246^{(a)(e)} | 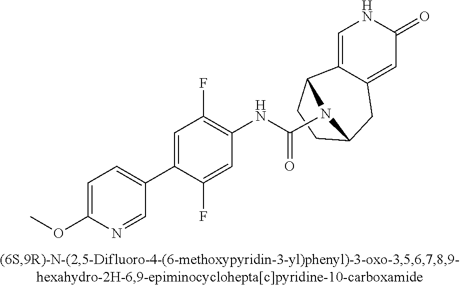 [SMILES: COc1ccc(-c2cc(F)c(NC(=O)N3[C@H]4CC[C@@H]3c3c[nH]c(=O)cc3C4)cc2F)cn1] | Method 1 439.3 at 1.15 min | ¹H NMR (400 MHz, DMSO-d6) δ 11.2 (s, 1 H), 8.61 (s, 1H), 8.35 (s, 1H), 7.90 (ddd, J = 8.7, 2.6, 1.6 Hz, 1H), 7.58 (dd, J = 12.4, 6.7 Hz, 1H), 7.47 (dd, J = 11.3, 7.2 Hz, 1H), 7.21 (s, 1H), 6.92 (dd, J = 8.6, 0.8 Hz, 1H), 6.11 (s, 1H), 5.08 (d, J = 5.8 Hz, 1H), 4.60 (t, J = 6.3 Hz, 1H), 3.89 (s, 3H), 3.14 (dd, J = 18.0, 5.0 Hz, 1H), 2.56 (d, J = 17.8 Hz, 1H), 2.23-2.08 (m, 2H), 1.69 (ddd, J = 22.1, 11.3, 7.0 Hz, 2H). |
| 248^{(a)(e)} | 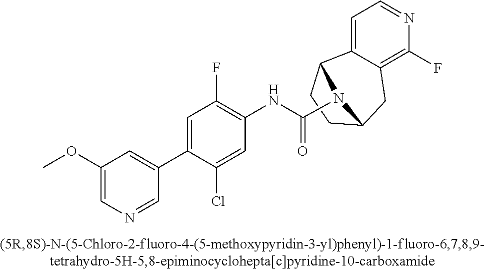 [SMILES: COc1cncc(-c2cc(F)c(NC(=O)N3[C@H]4CC[C@@H]3c3ccnc(F)c3C4)cc2Cl)c1] | Method 1 457.3, 459.3 at 1.44 min | ¹H NMR (400 MHz, DMSO-d6) δ 8.86 (s, 1H), 8.32 (d, J = 2.8 Hz, 1H), 8.22 (d, J = 1.8 Hz, 1H), 8.01 (d, J = 5.0 Hz, 1H), 7.78 (d, J = 7.3 Hz, 1H), 7.48-7.39 (m, 2H), 7.21 (dd, J = 5.0, 1.7 Hz, 1H), 5.24 (d, J = 5.9 Hz, 1H), 4.81 (t, J = 6.1 Hz, 1H), 3.87 (s, 3H), 3.21 (dd, J = 17.5, 4.9 Hz, 1H), 2.60 (d, J = 17.4 Hz, 1H), 2.29-2.16 (m, 2H), 1.90-1.81 (m, 1H), 1.81-1.73 (m, 1H). |
| 249^{(a)(e)} | 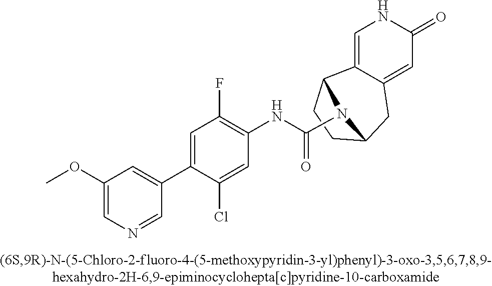 [SMILES: COc1cncc(-c2cc(F)c(NC(=O)N3[C@H]4CC[C@@H]3c3c[nH]c(=O)cc3C4)cc2Cl)c1] | Method 1 455.2, 457.2 at 1.05 min | ¹H NMR (400 MHz, DMSO-d6) δ 11.31 (s, 1H), 8.67 (s, 1H), 8.32 (d, J = 2.8 Hz, 1H), 8.23 (d, J = 1.8 Hz, 1H), 7.81 (d, J = 7.3 Hz, 1H), 7.48-7.40 (m, 2H), 7.21 (s, 1H), 6.11 (s, 1H), 5.07 (d, J = 5.8 Hz, 1H), 4.64-4.57 (m, 1H), 3.87 (s, 3H), 3.15 (dd, J = 17.9, 5.1 Hz, 1H), 2.56 (d, J = 17.9 Hz, 1H), 2.24-2.09 (m, 2H), 1.77-1.60 (m, 2H). |
| 250^{(a)(e)} | 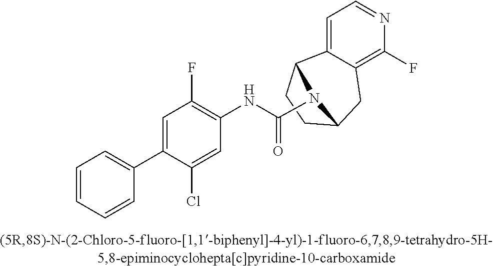 [SMILES: O=C(Nc1cc(Cl)c(-c2ccccc2)cc1F)N1[C@H]2CC[C@@H]1c1ccnc(F)c1C2] | Method 1 426.3, 428.3 at 1.74 min | ¹H NMR (400 MHz, DMSO-d6) δ 8.81 (s, 1H), 8.01 (d, J = 5.0 Hz, 1H), 7.73 (d, J = 7.3 Hz, 1H), 7.49-7.36 (m, 5H), 7.30 (d, J = 11.2 Hz, 1H), 7.21 (dd, J = 5.1, 1.6 Hz, 1H), 5.23 (d, J = 5.8 Hz, 1H), 4.84-4.76 (m, 1H), 3.21 (dd, J = 17.5, 4.9 Hz, 1H), 2.59 (d, J = 17.3 Hz, 1H), 2.30-2.17 (m, 2H), 1.90-1.81 (m, 1H), 1.81-1.73 (m, 1H). |
| 251^{(a)(e)} | 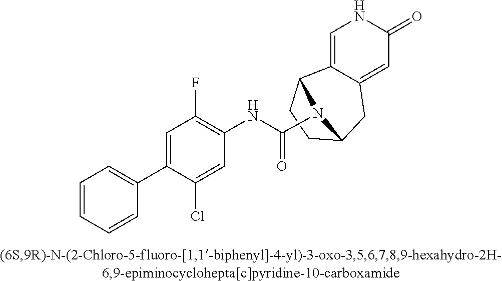 [SMILES: O=C(Nc1cc(Cl)c(-c2ccccc2)cc1F)N1[C@H]2CC[C@@H]1c1c[nH]c(=O)cc1C2] | Method 1 424.3, 426.3 at 1.34 min | ¹H NMR (400 MHz, DMSO-d6) δ 11.27 (s, 1H), 8.62 (s, 1H), 7.76 (d, J = 7.3 Hz, 1H), 7.51-7.36 (m, 5H), 7.30 (d, J = 11.1 Hz, 1H), 7.20 (s, 1H), 6.11 (s, 1H), 5.06 (d, J = 5.8 Hz, 1H), 4.60 (d, J = 7.1 Hz, 1H), 3.19-3.10 (m, 1H), 2.56 (d, J = 17.9 Hz, 1H), 2.19 (d, J = 9.3 Hz, 1H), 2.14-2.08 (m, 1H), 1.77-1.62 (m, 2H) |
| 252^{(a)(e)} | 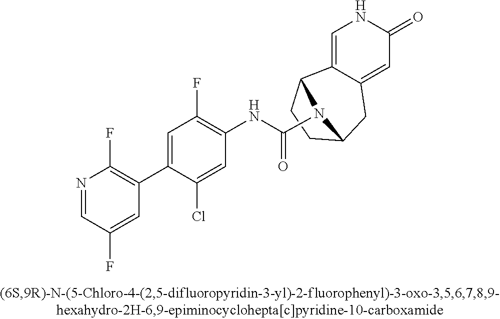 [SMILES: O=C(Nc1cc(Cl)c(-c2cc(F)cnc2F)cc1F)N1[C@H]2CC[C@@H]1c1c[nH]c(=O)cc1C2] | Method 1 461.1, 463.1 at 1.18 min | ¹H NMR (400 MHz, DMSO-d6) δ 11.28 (s, 1H), 8.71 (s, 1H), 8.35 (dd, J = 3.0, 1.7 Hz, 1H), 8.10 (td, J = 7.6, 3.0 Hz, 1H), 7.85 (d, J = 7.2 Hz, 1H), 7.50 (d, J = 10.9 Hz, 1H), 7.21 (s, 1H), 6.11 (s, 1H), 5.07 (d, J = 5.8 Hz, 1H), 4.61 (t, J = 6.3 Hz, 1H), 3.20-3.10 (m, 1H), 2.56 (d, J = 18.1 Hz, 1H), 2.24-2.02 (m, 2H), 1.77-1.60 (m, 2H). |
| 253^{(a)(e)} | 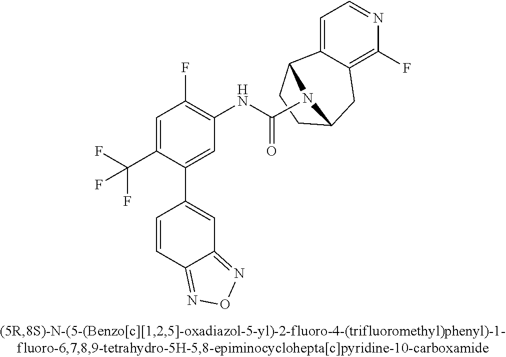 [SMILES: O=C(Nc1cc(-c2ccc3nonc3c2)c(C(F)(F)F)cc1F)N1[C@H]2CC[C@@H]1c1ccnc(F)c1C2] | Method 1 500.0 at 1.78 min | ¹H NMR (400 MHz, DMSO-d6) δ 9.12-9.08 (m, 1H), 8.12 (dd, J = 9.2, 1.1 Hz, 1H), 8.03-8.01 (m, 1H), 8.00 (d, J = 5.0 Hz, 1H), 7.82 (d, J = 3.7 Hz, 1H), 7.80 (s, 1H), 7.52 (d, J = 9.2 Hz, 1H), 7.19 (dd, J = 5.0, 1.6 Hz, 1H), 5.22 (d, J = 5.8 Hz, 1H), 4.81 (t, J = 6.2 Hz, 1H), 3.22-3.13 (m, 1H), 2.59 (d, J = 17.3 Hz, 1H), 2.31-2.12 (m, 2H), 1.89-1.80 (m, 1H), 1.80-1.70 (m, 1H). |
| 255^{(a)(e)} | 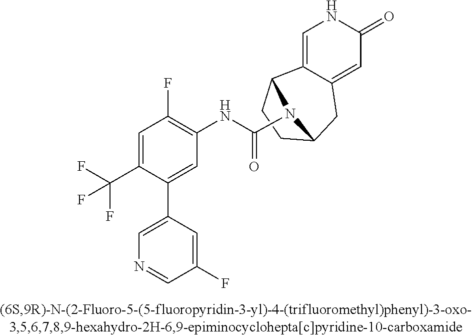 [SMILES: O=C(Nc1cc(-c2cncc(F)c2)c(C(F)(F)F)cc1F)N1[C@H]2CC[C@@H]1c1c[nH]c(=O)cc1C2] | Method 1 477.3 at 1.20 min | ¹H NMR (400 MHz, DMSO-d6) δ 11.29 (s, 1H), 8.89 (s, 1H), 8.67 (d, J = 2.8 Hz, 1H), 8.38 (d, J = 1.9 Hz, 1H), 7.84-7.74 (m, 3H), 7.20 (s, 1H), 6.09 (s, 1H), 5.06 (d, J = 5.8 Hz, 1H), 4.60 (t, J = 6.3 Hz, 1H), 3.19-3.07 (m, 1H), 2.55 (d, J = 17.7 Hz, 1H), 2.23-2.03 (m, 2H), 1.76-1.59 (m, 2H). |
| 256^{(a)(e)} | 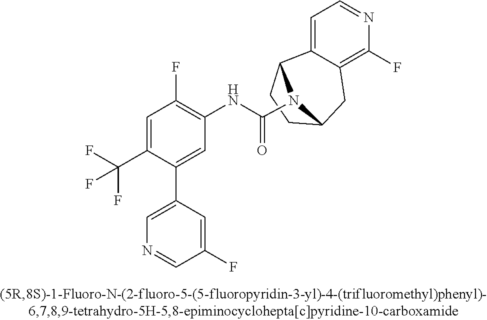 [SMILES: O=C(Nc1cc(-c2cncc(F)c2)c(C(F)(F)F)cc1F)N1[C@H]2CC[C@@H]1c1ccnc(F)c1C2] | Method 1 479.3 at 1.57 min | ¹H NMR (400 MHz, DMSO-d6) δ 9.08 (s, 1H), 8.67 (d, J = 2.8 Hz, 1H), 8.37 (d, J = 2.2 Hz, 1H), 8.00 (d, J = 5.0 Hz, 1H), 7.82-7.72 (m, 3H), 7.20 (dd, J = 5.0, 1.6 Hz, 1H), 5.22 (d, J = 5.9 Hz, 1H), 4.81 (d, J = 6.2 Hz, 1H), 3.18 (dd, J = 17.3, 4.9 Hz, 1H), 2.59 (d, J = 17.3 Hz, 1H), 2.28-2.14 (m, 2H), 1.84 (t, J = 10.0 Hz, 1H), 1.79-1.71 (m, 1H). |
| 259^{(a)(e)} | 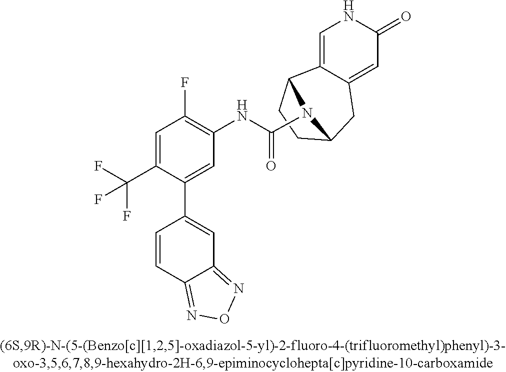 [SMILES: O=C(Nc1cc(-c2ccc3nonc3c2)c(C(F)(F)F)cc1F)N1[C@H]2CC[C@@H]1c1c[nH]c(=O)cc1C2] | Method 1 500.3 at 1.41 min | ¹H NMR (400 MHz, DMSO-d6) δ 11.29 (s, 1H), 8.91 (s, 1H), 8.12 (dd, J = 9.2, 1.1 Hz, 1H), 8.03 (d, J = 1.3 Hz, 1H), 7.87-7.79 (m, 2H), 7.53 (d, J = 9.3 Hz, 1H), 7.20 (s, 1H), 6.09 (s, 1H), 5.06 (d, J = 5.8 Hz, 1H), 4.60 (s, 1H), 3.12 (d, J = 13.4 Hz, 1H), 2.55 (d, J = 17.7 Hz, 1H), 2.17 (q, J = 11.2 Hz, 1H), 2.07 (s, 1H), 1.76-1.60 (m, 2H). |
| 260^{(a)(e)} | 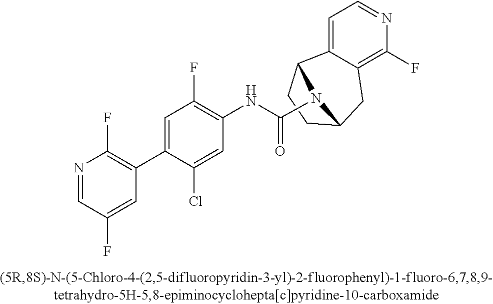 [SMILES: O=C(Nc1cc(Cl)c(-c2cc(F)cnc2F)cc1F)N1[C@H]2CC[C@@H]1c1ccnc(F)c1C2] | Method 1 463.2, 465.2 at 1.50 min | ¹H NMR (400 MHz, DMSO-d6) δ 8.91 (s, 1H), 8.35 (dd, J = 3.0, 1.7 Hz, 1H), 8.09 (td, J = 7.6, 3.0 Hz, 1H), 8.01 (d, J = 5.0 Hz, 1H), 7.82 (d, J = 7.2 Hz, 1H), 7.50 (d, J = 10.9 Hz, 1H), 7.21 (dd, J = 5.2, 1.7 Hz, 1H), 5.24 (d, J = 5.9 Hz, 1H), 4.81 (t, J = 6.2 Hz, 1H), 3.21 (dd, J = 17.4, 5.0 Hz, 1H), 2.60 (d, J = 17.4 Hz, 1H), 2.29-2.14 (m, 2H), 1.90-1.71 (m, 2H) |
| 261^{(a)(e)} | 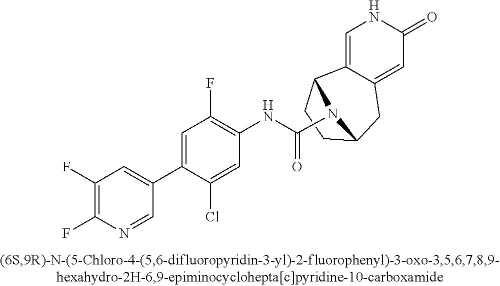 [SMILES: O=C(Nc1cc(Cl)c(-c2cnc(F)c(F)c2)cc1F)N1[C@H]2CC[C@@H]1c1c[nH]c(=O)cc1C2] | Method 1 461.3, 463.2 at 1.24 min | ¹H NMR (400 MHz, DMSO-d6) δ 11.27 (s, 1H), 8.70 (s, 1H), 8.25 (ddd, J = 10.6, 9.3, 2.0 Hz, 1H), 8.16 (t, J = 1.9 Hz, 1H), 7.84 (d, J = 7.2 Hz, 1H), 7.49 (d, J = 11.1 Hz, 1H), 7.21 (s, 1H), 6.11 (s, 1H), 5.07 (d, J = 5.8 Hz, 1H), 4.60 (t, J = 6.4 Hz, 1H), 3.15 (dd, J = 17.9, 5.1 Hz, 1H), 2.56 (d, J = 18.1 Hz, 1H), 2.25-2.03 (m, 2H), 1.80-1.55 (m, 2H). |
| 262^{(a)(e)} | 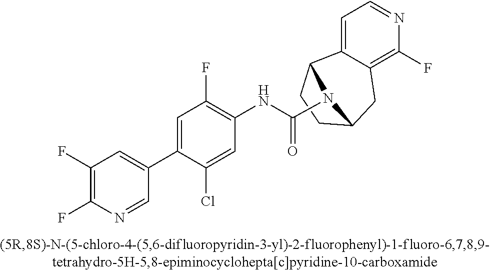 [SMILES: O=C(Nc1cc(Cl)c(-c2cnc(F)c(F)c2)cc1F)N1[C@H]2CC[C@@H]1c1ccnc(F)c1C2] | Method 2 463.1, 465.2 at 1.59 min | ¹H NMR (400 MHz, DMSO-d6) δ 8.89 (s, 1H), 8.29-8.19 (m, 1H), 8.15 (t, J = 1.9 Hz, 1H), 8.01 (d, J = 5.0 Hz, 1H), 7.82 (d, J = 7.3 Hz, 1H), 7.48 (d, J = 11.1 Hz, 1H), 5.24 (d, J = 5.9 Hz, 1H), 4.81 (t, J = 6.1 Hz, 1H), 3.20 (dd, J = 17.4, 4.9 Hz, 1H), 2.60 (d, J = 17.4 Hz, 2H), 2.32-2.15 (m, 2H), 1.93-1.71 (m, 2H). |
| 263^{(a)(e)} | 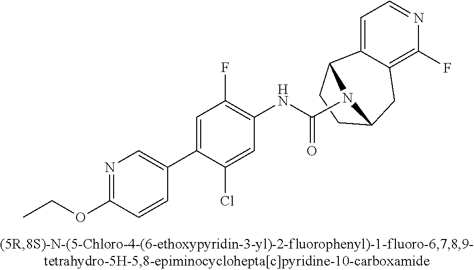 [SMILES: CCOc1ccc(-c2cc(F)c(NC(=O)N3[C@H]4CC[C@@H]3c3ccnc(F)c3C4)cc2Cl)cn1] | Method 1 471.2, 473.2 at 1.63 min | ¹H NMR (400 MHz, DMSO-d6) δ 8.82 (s, 1H), 8.19 (dd, J = 2.5, 0.8 Hz, 1H), 8.01 (d, J = 5.0 Hz, 1H), 7.82-7.71 (m, 2H), 7.36 (d, J = 11.1 Hz, 1H), 7.21 (dd, J = 5.1, 1.6 Hz, 1H), 6.87 (dd, J = 8.6, 0.8 Hz, 1H), 5.23 (d, J = 5.9 Hz, 1H), 4.80 (t, J = 6.1 Hz, 1H), 4.34 (q, J = 7.0 Hz, 2H), 3.20 (dd, J = 17.4, 5.0 Hz, 1H), 2.59 (d, J = 17.4 Hz, 1H), 2.33-2.14 (m, 2H), 1.90-1.71 (m, 2H), 1.33 (t, J = 7.1 Hz, 3H). |
| 264^{(a)(e)} | 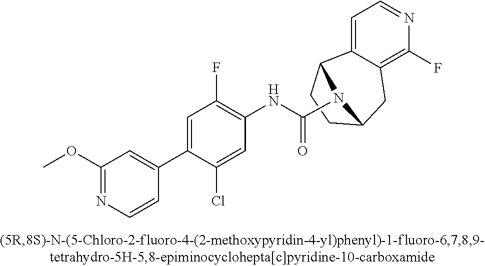 [SMILES: COc1cc(-c2cc(F)c(NC(=O)N3[C@H]4CC[C@@H]3c3ccnc(F)c3C4)cc2Cl)ccn1] | Method 1 457.3, 459.3 at 1.60 min | ¹H NMR (400 MHz, DMSO-d6) δ 8.90 (s, 1H), 8.26-8.20 (m, 1H), 8.01 (d, J = 4.9 Hz, 1H), 7.78 (d, J = 7.3 Hz, 1H), 7.39 (d, J = 11.0 Hz, 1H), 7.24-7.18 (m, 1H), 7.05 (dd, J = 5.3, 1.5 Hz, 1H), 6.86 (d, J = 1.3 Hz, 1H), 5.23 (d, J = 5.9 Hz, 1H), 4.81 (d, J = 6.3 Hz, 1H), 3.88 (s, 3H), 3.20 (dd, J = 17.5, 5.0 Hz, 1H), 2.59 (d, J = 17.3 Hz, 1H), 2.30-2.16 (m, 2H), 1.85 (t, J = 9.9 Hz, 1H), 1.76 (s, 1H). |
| 265^{(a)(e)} | 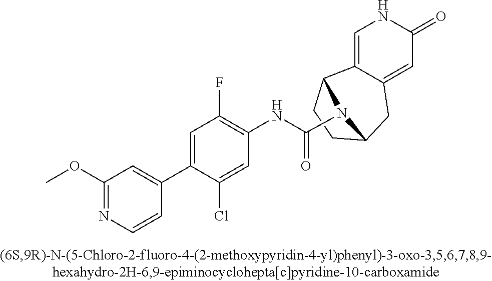 [SMILES: COc1cc(-c2cc(F)c(NC(=O)N3[C@H]4CC[C@@H]3c3c[nH]c(=O)cc3C4)cc2Cl)ccn1] | Method 1 455.3, 457.3 at 1.19 min | ¹H NMR (400 MHz, DMSO-d6) δ 11.31 (s, 1H), 8.70 (s, 1H), 8.23 (d, J = 5.3 Hz, 1H), 7.81 (d, J = 7.2 Hz, 1H), 7.39 (d, J = 11.1 Hz, 1H), 7.21 (s, 1H), 7.06 (dd, J = 5.3, 1.5 Hz, 1H), 6.87 (d, J = 1.4 Hz, 1H), 6.11 (s, 1H), 5.06 (d, J = 5.8 Hz, 1H), 4.60 (t, J = 6.3 Hz, 1H), 3.89 (s, 3H), 3.17-3.11 (m, 1H), 2.59-2.53 (m, 1H), 2.24-2.08 (m, 2H), 1.77-1.59 (m, 2H). |
| 267^{(a)(e)} | 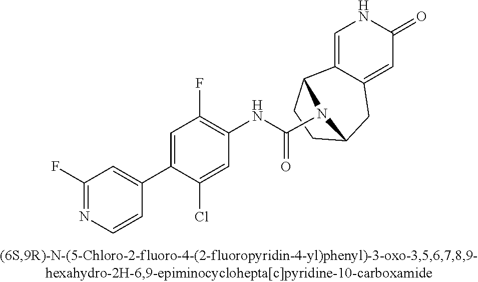 [SMILES: O=C(Nc1cc(Cl)c(-c2ccnc(F)c2)cc1F)N1[C@H]2CC[C@@H]1c1c[nH]c(=O)cc1C2] | Method 1 443.2, 445.2 at 1.13 min | ¹H NMR (400 MHz, DMSO-d6) δ 11.31 (s, 1H), 8.74 (s, 1H), 8.33 (d, J = 5.2 Hz, 1H), 7.86 (d, J = 7.2 Hz, 1H), 7.53-7.44 (m, 2H), 7.32 (d, J = 1.6 Hz, 1H), 7.21 (s, 1H), 6.11 (s, 1H), 5.07 (d, J = 5.8 Hz, 1H), 4.60 (t, J = 6.4 Hz, 1H), 3.19-3.10 (m, 1H), 2.56 (d, J = 17.8 Hz, 1H), 2.24-2.08 (m, 2H), 1.77-1.60 (m, 2H). |
| 268^{(a)(e)} | 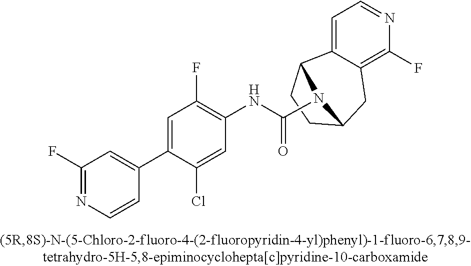 [SMILES: O=C(Nc1cc(Cl)c(-c2ccnc(F)c2)cc1F)N1[C@H]2CC[C@@H]1c1ccnc(F)c1C2] | Method 1 443.3, 445.2 at 1.54 min | ¹H NMR (400 MHz, DMSO-d6) δ 8.94 (s, 1H), 8.33 (d, J = 5.2 Hz, 1H), 8.01 (d, J = 5.0 Hz, 1H), 7.83 (d, J = 7.2 Hz, 1H), 7.51-7.45 (m, 2H), 7.32 (d, J = 1.7 Hz, 1H), 7.22 (dd, J = 5.0, 1.6 Hz, 1H), 5.24 (d, J = 5.9 Hz, 1H), 4.81 (t, J = 6.1 Hz, 1H), 3.20 (dd, J = 17.6, 5.1 Hz, 1H), 2.60 (d, J = 17.3 Hz, 1H), 2.31-2.15 (m, 2H), 1.85 (t, J = 10.0 Hz, 1H), 1.77 (dd, J = 12.0, 5.4 Hz, 1H). |
| 272^{(a)(e)} | 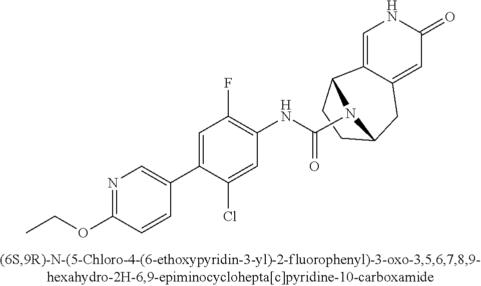 [SMILES: CCOc1ccc(-c2cc(F)c(NC(=O)N3[C@H]4CC[C@@H]3c3c[nH]c(=O)cc3C4)cc2Cl)cn1] | Method 1 443.3, 445.2 at 1.30 min | ¹H NMR (400 MHz, DMSO-d6) δ 11.27 (s, 1H), 8.63 (s, 1H), 8.20 (d, J = 2.5 Hz, 1H), 7.83-7.73 (m, 2H), 7.36 (d, J = 11.1 Hz, 1H), 7.20 (s, 1H), 6.87 (d, J = 8.6 Hz, 1H), 6.11 (s, 1H), 5.06 (d, J = 5.8 Hz, 1H), 4.59 (t, J = 6.3 Hz, 1H), 4.34 (q, J = 7.0 Hz, 2H), 3.15 (dd, J = 17.9, 5.1 Hz, 1H), 2.56 (d, J = 17.9 Hz, 1H), 2.25-2.02 (m, 2H), 1.77-1.60 (m, 2H), 1.34 (t, J = 7.0 Hz, 3H). |
| 273^{(a)(e)} | 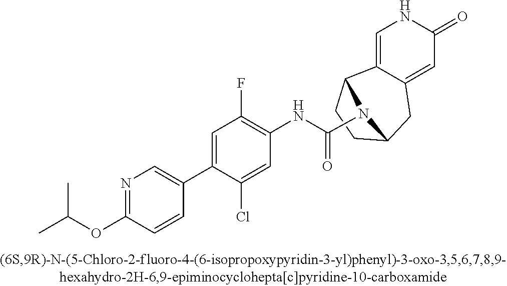 [SMILES: CC(C)Oc1ccc(-c2cc(F)c(NC(=O)N3[C@H]4CC[C@@H]3c3c[nH]c(=O)cc3C4)cc2Cl)cn1] | Method 1 483.2, 485.2 at 1.41 min | ¹H NMR (400 MHz, DMSO-d6) δ 11.26 (s, 1H), 8.63 (s, 1H), 8.19 (d, J = 2.5 Hz, 1H), 7.81-7.73 (m, 2H), 7.36 (d, J = 11.1 Hz, 1H), 7.20 (s, 1H), 6.81 (d, J = 8.6 Hz, 1H), 6.11 (s, 1H), 5.28 (hept, J = 6.2 Hz, 1H), 5.06 (d, J = 5.8 Hz, 1H), 4.66-4.56 (m, 1H), 3.15 (dd, J = 18.0, 5.1 Hz, 1H), 2.55 (d, J = 17.9 Hz, 1H), 2.24-2.02 (m, 2H), 1.77-1.60 (m, 2H), 1.31 (d, J = 6.1 Hz, 6H) |
| 274^{(a)(e)} | 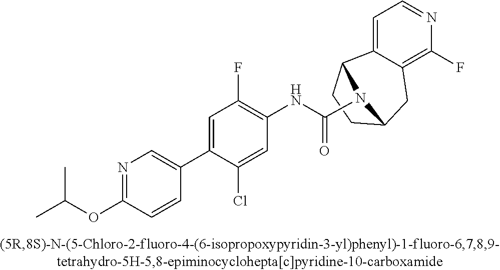 [SMILES: CC(C)Oc1ccc(-c2cc(F)c(NC(=O)N3[C@H]4CC[C@@H]3c3ccnc(F)c3C4)cc2Cl)cn1] | Method 1 485.2, 487.2 at 1.73 min | ¹H NMR (400 MHz, DMSO-d6) δ 8.83 (s, 1H), 8.19 (d, J = 2.5 Hz, 1H), 8.01 (d, J = 5.0 Hz, 1H), 7.80-7.71 (m, 2H), 7.36 (d, J = 11.1 Hz, 1H), 7.21 (dd, J = 5.1, 1.6 Hz, 1H), 6.81 (d, J = 8.6 Hz, 1H), 5.35-5.19 (m, 2H), 4.80 (t, J = 6.0 Hz, 1H), 3.20 (dd, J = 17.5, 5.0 Hz, 1H), 2.59 (d, J = 17.4 Hz, 1H), 2.33-2.14 (m, 2H), 1.90-1.70 (m, 2H), 1.31 (d, J = 6.1 Hz, 6H) |
| 276^{(a)(e)} | 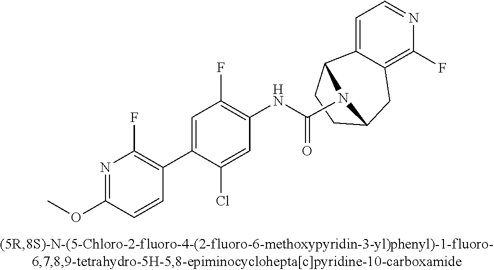 [SMILES: COc1ccc(-c2cc(F)c(NC(=O)N3[C@H]4CC[C@@H]3c3ccnc(F)c3C4)cc2Cl)c(F)n1] | Method 1 475.3, 477.2 at 1.70 min | ¹H NMR (400 MHz, DMSO-d6) δ 8.87 (s, 1H), 8.01 (d, J = 4.9 Hz, 1H), 7.86 (dd, J = 10.0, 8.2 Hz, 1H), 7.76 (d, J = 7.3 Hz, 1H), 7.39 (d, J = 11.0 Hz, 1H), 7.21 (dd, J = 5.0, 1.5 Hz, 1H), 6.88 (dd, J = 8.1, 1.2 Hz, 1H), 5.23 (d, J = 5.9 Hz, 1H), 4.82-4.78 (m, 1H), 3.88 (s, 3H), 3.24-3.18 (m, 1H), 2.59 (d, J = 17.4 Hz, 1H), 2.30-2.16 (m, 2H), 1.88-1.81 (m, 1H), 1.80-1.72 (m, 1H). |
| 284^{(a)(e)} | 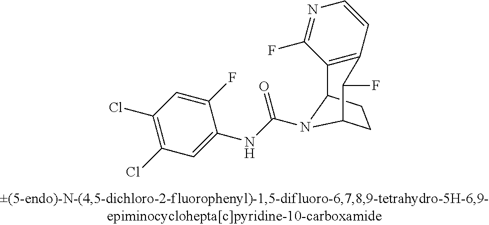 [SMILES: O=C(Nc1cc(Cl)c(Cl)cc1F)N1C2CCC1C(F)c1ccnc(F)c12] | Method 3 402.0, 404.0 at 1.69 min | ¹H NMR (400 MHz, DMSO-d6) δ 9.19 (s, 1H), 8.22 (d, J = 5.1 Hz, 1H), 7.87 (dd, J = 7.6, 3.4 Hz, 1H), 7.71 (d, J = 10.4 Hz, 1H), 7.46 (d, J = 5.1 Hz, 1H), 6.14 (dd, J = 49.9, 5.4 Hz, 1H), 5.47-5.34 (m, 1H), 4.98 (d, J = 6.1 Hz, 1H), 2.26-2.11 (m, 2H), 2.11-1.92 (m, 1H), 1.91-1.76 (m, 1H). |
| 289^{(a)(d)} | 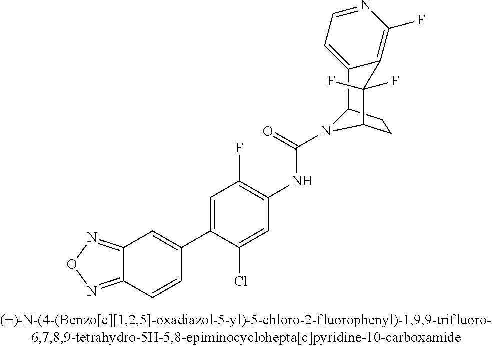 [SMILES: O=C(Nc1cc(Cl)c(-c2ccc3nonc3c2)cc1F)N1C2CCC1C(F)(F)c1c2ccnc1F] | Method 4 504.1 at 2.14 min | ¹H NMR (400 MHz, DMSO-d6) δ 9.21 (s, 1H), 8.39 (d, J = 5.2 Hz, 1H), 8.15-8.11 (m, 2H), 7.83 (d, J = 7.2 Hz, 1H), 7.69 (dd, J = 1.2, 9.2 Hz, 1H), 7.58-7.50 (m, 2H), 5.48 (d, J = 6.4 Hz, 1H), 5.29-5.24 (m, 1H), 2.39-2.31 (m, 1H), 2.23-2.15 (m, 1H), 1.98-1.90 (m, 1H), 1.75-1.70 (m, 1H). |
| 290^{(a)(d)} | 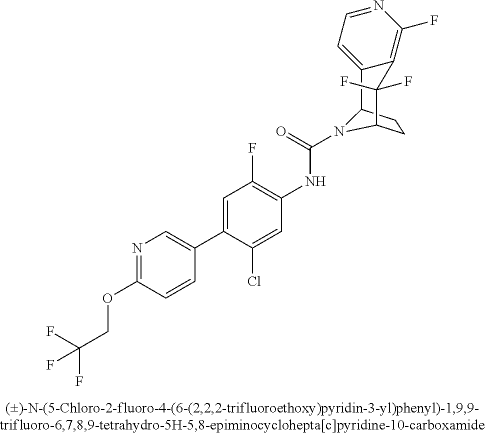 [SMILES: O=C(Nc1cc(Cl)c(-c2ccc(OCC(F)(F)F)nc2)cc1F)N1C2CCC1C(F)(F)c1c2ccnc1F] | Method 3 560.9 at 2.81 min | ¹H NMR (400 MHz, DMSO-d6) δ 9.14 (s, 1H), 8.38 (d, J = 5.2 Hz, 1H), 8.27 (d, J = 2.4 Hz, 1H), 7.93 (dd, J = 8.8, 2.8 Hz, 1H), 7.76 (d, J = 7.2 Hz, 1H), 7.50 (d, J = 5.2 Hz, 1H), 7.43 (d, J = 11.2 Hz, 1H), 7.08 (d, J = 8.4 Hz, 1H), 5.46 (d, J = 6.4 Hz, 1H), 5.27-5.22 (m, 1H), 5.05 (q, J = 9.2 Hz, 2H), 2.38-2.29 (m, 1H), 2.24-2.14 (m, 1H), 1.93-1.88 (m, 1H), 1.75-1.69 (m, 1H). |
| 297^{(a)(e)} | 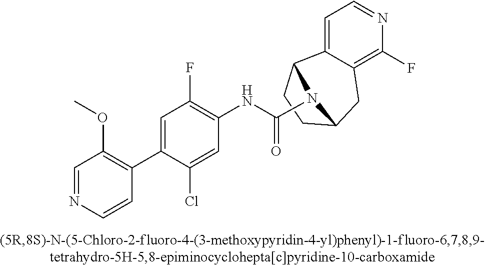 [SMILES: COc1cnccc1-c1cc(F)c(NC(=O)N2[C@H]3CC[C@@H]2c2ccnc(F)c2C3)cc1Cl] | Method 1 457.2, 459.2 at 1.31 min | ¹H NMR (500 MHz, DMSO-d6) δ 8.84 (s, 1H), 8.48 (s, 1H), 8.28 (d, J = 4.7 Hz, 1H), 8.01 (d, J = 5.0 Hz, 1H), 7.71 (d, J = 7.2 Hz, 1H), 7.27 (d, J = 10.9 Hz, 1H), 7.24-7.18 (m, 2H), 5.22 (d, J = 6.1 Hz, 1H), 4.80 (t, J = 6.2 Hz, 1H), 3.85 (s, 3H), 3.21 (dd, J = 17.5, 5.0 Hz, 1H), 2.59 (d, J = 17.4 Hz, 1H), 2.32-2.15 (m, 2H), 1.90-1.81 (m, 1H), 1.81-1.72 (m, 1H). |
| 298^{(a)(e)} | 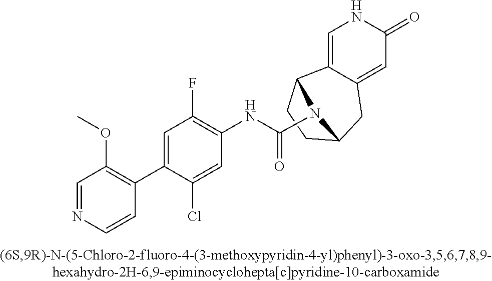 [SMILES: COc1cnccc1-c1cc(F)c(NC(=O)N2[C@H]3CC[C@@H]2c2c[nH]c(=O)cc2C3)cc1Cl] | Method 1 455.1, 457.2 at 1.00 min | ¹H NMR (500 MHz, DMSO-d6) δ 11.28 (s, 1H), 8.64 (s, 1H), 8.48 (s, 1H), 8.29 (d, J = 4.7 Hz, 1H), 7.74 (d, J = 7.2 Hz, 1H), 7.28 (d, J = 10.8 Hz, 1H), 7.23 (d, J = 4.7 Hz, 1H), 7.20 (s, 1H), 6.11 (s, 1H), 5.06 (d, J = 6.0 Hz, 1H), 4.62-4.56 (m, 1H), 3.85 (s, 3H), 3.15 (dd, J = 18.2, 4.9 Hz, 1H), 2.56 (d, J = 18.2 Hz, 1H), 2.24-2.03 (m, 2H), 1.76-1.61 (m, 2H). |
| 301^{(a)(e)} | 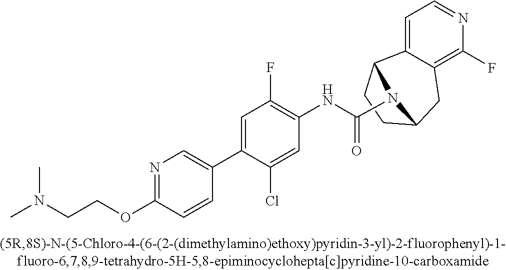 [SMILES: CN(C)CCOc1ccc(-c2cc(F)c(NC(=O)N3[C@H]4CC[C@@H]3c3ccnc(F)c3C4)cc2Cl)cn1] | Method 1 4.3, 516.3 at 1.53 min | ¹H NMR (400 MHz, DMSO-d6) δ 8.85 (s, 1H), 8.21-8.19 (m, 1H), 8.01 (d, J = 5.0 Hz, 1H), 7.79 (dd, J = 8.6, 2.6 Hz, 1H), 7.74 (d, J = 7.5 Hz, 1H), 7.37 (d, J = 11.1 Hz, 1H), 7.21 (dd, J = 5.1, 1.7 Hz, 1H), 6.91-6.86 (m, 1H), 5.22 (d, J = 6.0 Hz, 1H), 4.83-4.76 (m, 1H), 4.38 (t, J = 5.8 Hz, 2H), 3.23-3.17 (m, 1H), 2.66 (t, J = 5.8 Hz, 2H), 2.59 (d, J = 17.1 Hz, 1H), 2.26-2.19 (m, 8H), 1.89-1.81 (m, 1H), 1.80-1.72 (m, 1H). |
| 309^{(a)(c)} | 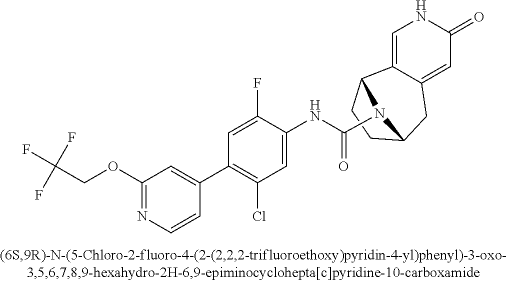 [SMILES: O=C(Nc1cc(Cl)c(-c2ccnc(OCC(F)(F)F)c2)cc1F)N1[C@H]2CC[C@@H]1c1c[nH]c(=O)cc1C2] | Method 1 523.3, 525.2 at 1.47 min | ¹H NMR (400 MHz, DMSO-d6) δ 11.29 (s, 1H), 8.71 (s, 1H), 8.27 (dd, J = 5.3, 0.7 Hz, 1H), 7.83 (d, J = 7.3 Hz, 1H), 7.44 (d, J = 11.1 Hz, 1H), 7.25-7.15 (m, 2H), 7.06 (dd, J = 1.5, 0.8 Hz, 1H), 6.11 (s, 1H), 5.20-4.92 (m, 3H), 4.60 (t, J = 6.4 Hz, 1H), 3.15 (dd, J = 18.0, 5.2 Hz, 1H), 2.56 (d, J = 17.9 Hz, 1H), 2.25-2.00 (m, 2H), 1.79-1.60 (m, 2H). |
| 312^{(a)(c)} | 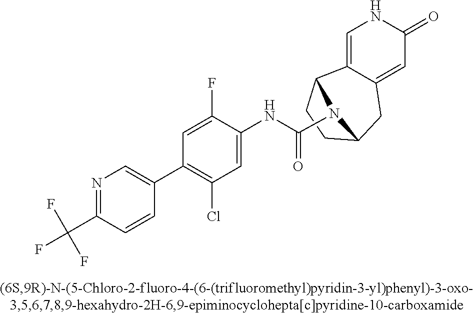 [SMILES: O=C(Nc1cc(Cl)c(-c2ccc(C(F)(F)F)nc2)cc1F)N1[C@H]2CC[C@@H]1c1c[nH]c(=O)cc1C2] | Method 1 493.3, 495.2 at 1.33 min | ¹H NMR (400 MHz, DMSO-d6) δ 11.29 (s, 1H), 8.85 (d, J = 2.2 Hz, 1H), 8.74 (s, 1H), 8.29-8.12 (m, 1H), 8.02 (dd, J = 8.3, 0.8 Hz, 1H), 7.88 (d, J = 7.3 Hz, 1H), 7.54 (d, J = 11.0 Hz, 1H), 7.21 (s, 1H), 6.11 (s, 1H), 5.08 (d, J = 5.8 Hz, 1H), 4.61 (t, J = 6.2 Hz, 1H), 3.22-3.08 (m, 1H), 2.56 (d, J = 17.9 Hz, 1H), 2.25-2.00 (m, 1H), 1.86-1.53 (m, 2H), One exchangeable proton not observed |
| 317^{(a)(c)} | 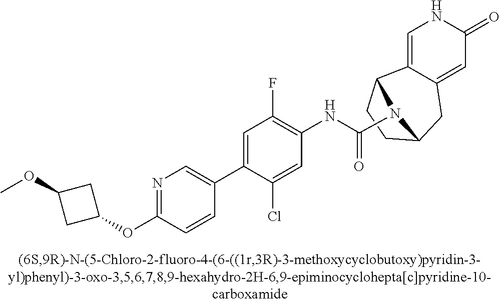 [SMILES: CO[C@H]1C[C@H](Oc2ccc(-c3cc(F)c(NC(=O)N4[C@H]5CC[C@@H]4c4c[nH]c(=O)cc4C5)cc3Cl)cn2)C1] | Method 2 525.4, 527.5 at 1.65 min | ¹H NMR (400 MHz, DMSO-d6) δ 11.28 (s, 1H), 8.64 (s, 1H), 8.24-8.15 (m, 1H), 7.80 (dd, J = 8.6, 2.6 Hz, 1H), 7.76 (d, J = 7.3 Hz, 1H), 7.37 (d, J = 11.1 Hz, 1H), 7.20 (s, 1H), 6.88 (dd, J = 8.6, 0.8 Hz, 1H), 6.11 (s, 1H), 5.36-5.21 (m, 1H), 5.06 (d, J = 5.7 Hz, 1H), 4.59 (t, J = 6.0 Hz, 1H), 4.11-4.05 (m, 1H), 3.17 (s, 3H), 3.12 (d, J = 4.8 Hz, 1H), 2.55 (d, J = 18.1 Hz, 1H), 2.45-2.37 (m, 2H), 2.37-2.25 (m, 2H), 2.25-2.13 (m, 1H), 2.13-2.02 (m, 1H), 1.77-1.56 (m, 2H). |
| 321^{(a)(c)} | 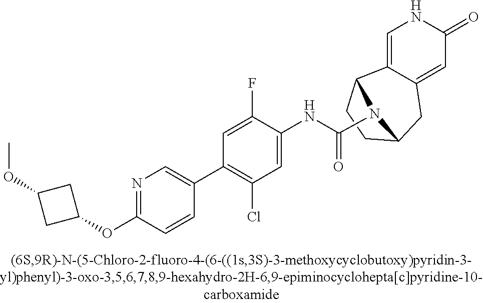 [SMILES: CO[C@H]1C[C@@H](Oc2ccc(-c3cc(F)c(NC(=O)N4[C@H]5CC[C@@H]4c4c[nH]c(=O)cc4C5)cc3Cl)cn2)C1] | Method 1 525.5, 527.4 at 1.31 min | ¹H NMR (400 MHz, DMSO-d6) δ 11.28 (s, 1H), 8.64 (s, 1H), 8.18 (dd, J = 2.5, 0.8 Hz, 1H), 7.80 (dd, J = 8.6, 2.5 Hz, 1H), 7.76 (d, J = 7.4 Hz, 1H), 7.37 (d, J = 11.1 Hz, 1H), 7.20 (s, 1H), 6.88 (dd, J = 8.6, 0.8 Hz, 1H), 6.11 (s, 1H), 5.06 (d, J = 5.8 Hz, 1H), 4.84 (p, J = 7.2 Hz, 1H), 4.59 (t, J = 6.2 Hz, 1H), 3.65 (p, J = 6.9 Hz, 1H), 3.16 (s, 4H), 2.89-2.77 (m, 2H), 2.55 (d, J = 17.9 Hz, 1H), 2.26-2.01 (m, 2H), 1.99-1.86 (m, 2H), 1.80-1.55 (m, 2H). |
| 322^{(a)(c)} | 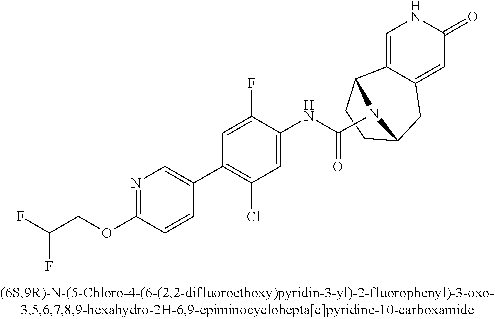 [SMILES: O=C(Nc1cc(Cl)c(-c2ccc(OCC(F)F)nc2)cc1F)N1[C@H]2CC[C@@H]1c1c[nH]c(=O)cc1C2] | Method 1 505.3, 507.3 at 1.35 min | ¹H NMR (400 MHz, DMSO-d6) δ 11.29 (s, 1H), 8.66 (s, 1H), 8.24 (dd, J = 2.5, 0.8 Hz, 1H), 7.87 (dd, J = 8.6, 2.5 Hz, 1H), 7.78 (d, J = 7.3 Hz, 1H), 7.39 (d, J = 11.1 Hz, 1H), 7.21 (s, 1H), 7.02 (dd, J = 8.6, 0.8 Hz, 1H), 6.42 (tt, J = 54.6, 3.5 Hz, 1H), 6.11 (s, 1H), 5.06 (d, J = 5.9 Hz, 1H), 4.73-4.52 (m, 3H), 3.14 (dd, J = 17.3, 4.9 Hz, 1H), 2.56 (d, J = 17.9 Hz, 1H), 2.26-2.01 (m, 2H), 1.81-1.57 (m, 2H). |
| 324^{(a)(c)} | 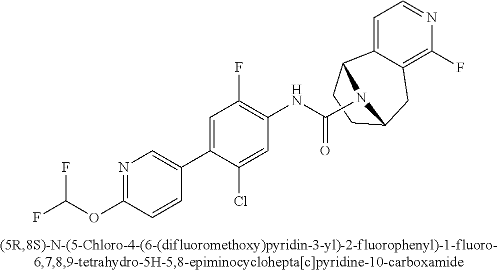 [SMILES: O=C(Nc1cc(Cl)c(-c2ccc(OC(F)F)nc2)cc1F)N1[C@H]2CC[C@@H]1c1ccnc(F)c1C2] | Method 2 493.3, 495.4 at 2.08 min | ¹H NMR (400 MHz, DMSO-d6) δ 8.87 (s, 1H), 8.33 (dd, J = 2.5, 0.7 Hz, 1H), 8.06-7.98 (m, 2H), 7.79 (d, J = 7.3 Hz, 1H), 7.76 (t, J = 72.7 Hz, 1H), 7.43 (d, J = 11.0 Hz, 1H), 7.24-7.15 (m, 2H), 5.23 (d, J = 5.9 Hz, 1H), 4.81 (t, J = 6.2 Hz, 1H), 3.20 (dd, J = 17.9, 5.2 Hz, 1H), 2.59 (d, J = 17.3 Hz, 1H), 2.33-2.14 (m, 2H), 1.90-1.72 (m, 2H). |
| 325^{(a)(c)} | 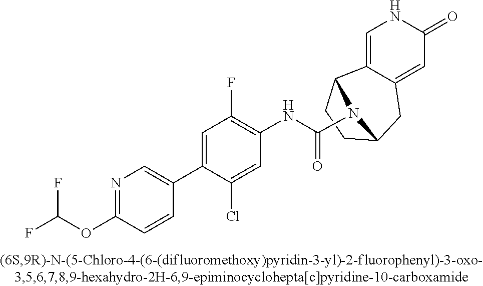 [SMILES: O=C(Nc1cc(Cl)c(-c2ccc(OC(F)F)nc2)cc1F)N1[C@H]2CC[C@@H]1c1c[nH]c(=O)cc1C2] | Method 1 491.1, 493.4 at 1.36 min | ¹H NMR (400 MHz, DMSO-d6) δ 8.70 (s, 1H), 8.33 (d, J = 2.5 Hz, 1H), 8.03 (dd, J = 8.5, 2.5 Hz, 1H), 7.81 (d, J = 7.3 Hz, 1H), 7.76 (t, J = 72.7 Hz, 1H), 7.43 (d, J = 11.0 Hz, 1H), 7.29 (s, 1H), 7.22-7.16 (m, 1H), 6.19 (s, 1H), 5.09 (d, J = 5.8 Hz, 1H), 4.61 (t, J = 6.4 Hz, 1H), 3.17 (dd, J = 17.9, 5.1 Hz, 1H), 2.59 (d, J = 18.0 Hz, 1H), 2.22-2.03 (m, 2H), 1.78-1.60 (m, 2H). 1 exchangeable proton not observed |
| 332^{(a)(c)} | 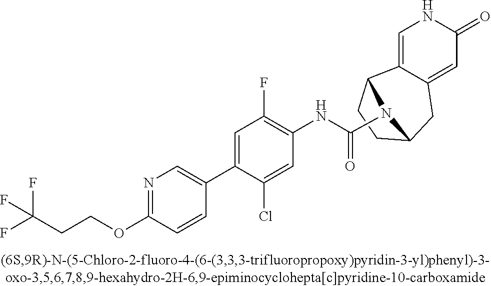 [SMILES: O=C(Nc1cc(Cl)c(-c2ccc(OCCC(F)(F)F)nc2)cc1F)N1[C@H]2CC[C@@H]1c1c[nH]c(=O)cc1C2] | Method 1 537.3, 539.3 at 1.46 min | ¹H NMR (400 MHz, DMSO-d6) δ 11.28 (s, 1H), 8.65 (s, 1H), 8.23 (dd, J = 2.6, 0.8 Hz, 1H), 7.83 (dd, J = 8.6, 2.5 Hz, 1H), 7.77 (d, J = 7.3 Hz, 1H), 7.38 (d, J = 11.1 Hz, 1H), 7.20 (s, 1H), 6.92 (dd, J = 8.6, 0.8 Hz, 1H), 6.11 (s, 1H), 5.06 (d, J = 5.9 Hz, 1H), 4.59 (t, J = 6.4 Hz, 1H), 4.53 (t, J = 6.0 Hz, 2H), 3.14 (dd, J = 17.7, 4.7 Hz, 1H), 2.81 (dp, J = 17.3, 5.7 Hz, 2H), 2.56 (d, J = 17.9 Hz, 1H), 2.22-2.05 (m, 2H), 1.77-1.60 (m, 2H). |
| 334^{(a)(c)} | 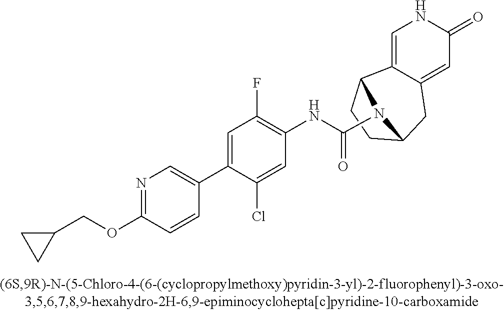 [SMILES: O=C(Nc1cc(Cl)c(-c2ccc(OCC3CC3)nc2)cc1F)N1[C@H]2CC[C@@H]1c1c[nH]c(=O)cc1C2] | Method 1 495.1, 497.4 at 1.44 min | ¹H NMR (400 MHz, DMSO-d6) δ 11.27 (s, 1H), 8.62 (s, 1H), 8.18 (dd, J = 2.5, 0.8 Hz, 1H), 7.81-7.73 (m, 2H), 7.36 (d, J = 11.1 Hz, 1H), 7.20 (s, 1H), 6.90 (dd, J = 8.6, 0.7 Hz, 1H), 6.11 (s, 1H), 5.06 (d, J = 5.8 Hz, 1H), 4.59 (t, J = 6.4 Hz, 1H), 4.13 (d, J = 7.2 Hz, 2H), 3.24-3.03 (m, 1H), 2.56 (d, J = 17.9 Hz, 1H), 2.26-2.00 (m, 2H), 1.79-1.55 (m, 2H), 1.34-1.18 (m, 1H), 0.62-0.49 (m, 2H), 0.41-0.25 (m, 2H). |
| 335^{(a)(c)} | 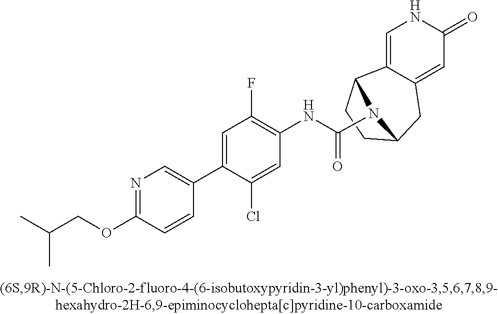 [SMILES: CC(C)COc1ccc(-c2cc(F)c(NC(=O)N3[C@H]4CC[C@@H]3c3c[nH]c(=O)cc3C4)cc2Cl)cn1] | Method 1 497.4, 499.3 at 1.57 min | ¹H NMR (400 MHz, DMSO-d6) δ 11.26 (s, 1H), 8.63 (s, 1H), 8.19 (dd, J = 2.5, 0.8 Hz, 1H), 7.87-7.68 (m, 2H), 7.36 (d, J = 11.1 Hz, 1H), 7.20 (s, 1H), 6.89 (dd, J = 8.6, 0.8 Hz, 1H), 6.11 (s, 1H), 5.06 (d, J = 5.8 Hz, 1H), 4.60 (t, J = 6.4 Hz, 1H), 4.07 (d, J = 6.7 Hz, 2H), 3.15 (dd, J = 17.2, 5.0 Hz, 1H), 2.56 (d, J = 18.0 Hz, 1H), 2.25-1.96 (m, 3H), 1.79-1.57 (m, 2H), 0.98 (d, J = 6.7 Hz, 6H). |
| 339^{(a)(e)} | 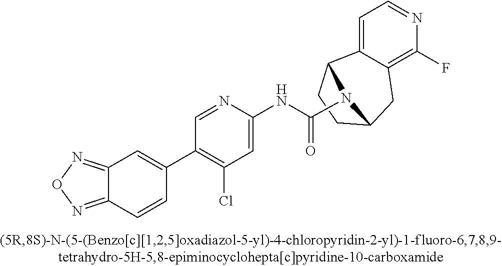 [SMILES: O=C(Nc1cc(Cl)c(-c2ccc3nonc3c2)cn1)N1[C@H]2CC[C@@H]1c1ccnc(F)c1C2] | Method 1 350.8, 352.1 at 1.65 min | ¹H NMR (400 MHz, DMSO-d6) δ 10.01 (s, 1H), 8.44 (s, 1H), 8.19 (t, J = 1.3 Hz, 1H), 8.14 (dd, J = 9.3, 1.1 Hz, 1H), 8.12 (s, 1H), 8.02 (d, J = 5.0 Hz, 1H), 7.72 (dd, J = 9.3, 1.4 Hz, 1H), 7.31-7.14 (m, 1H), 5.39 (s, 1H), 4.93 (s, 1H), 3.19 (d, J = 16.1 Hz, 1H), 2.60 (d, J = 17.4 Hz, 1H), 2.30-2.13 (m, 2H), 1.94-1.81 (m, 1H), 1.81-1.66 (m, 1H). |

(a)reaction performed with DMAP instead of Et₃N and DIPEA,
^{(b)}reaction performed with just Et₃N,
^{(c)}product purified by chromatograpphy on silica get (either 1-100% EtOAc/isohexane, 0-15% MeOH/DCM, 0-15% (0.7M NH₃ in MeOH/DCM or 0-100% 3:1 EtOAc:EtOH/isohexane
^{(d)}product purified by prep TLC (in EtOAc/petroleum ether,
^{(e)}Product purified by mass directed reverse phase prep HPLC

### Intermediate 3 (I-3)

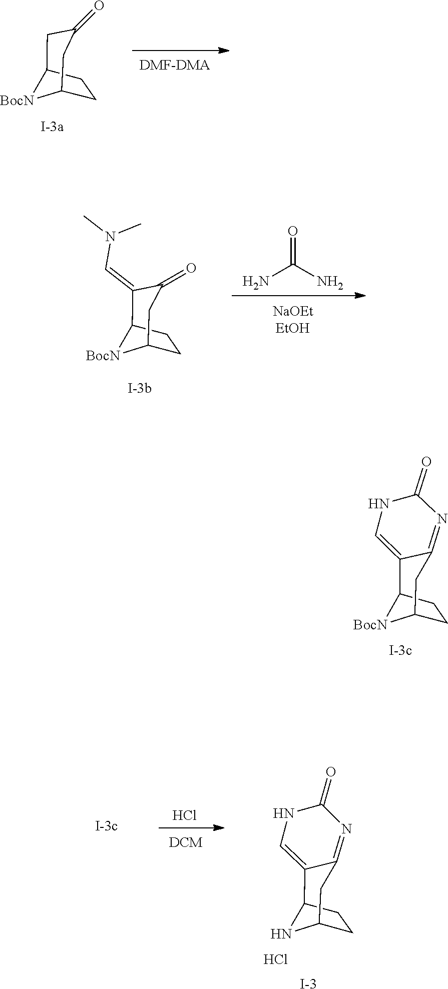

Step 1: tert-Butyl 3-oxo-8-azabicyclo[3.2.1]octane-8-carboxylate I-3a (10 g, 44 mmol) was dissolved in 1,1-dimethoxy-N,N-dimethylmethanamine (24 ml, 0.18 mol) and the reaction mixture was heated at reflux for 16 h. The solvent was removed in vacuo. The product was purified by silica gel chromatography (0-5% (0.7 M ammonia/MeOH) in DCM) to afford tert-butyl (E)-2-((dimethylamino)methylene)-3-oxo-8-azabicyclo[3.2.1]octane-8-carboxylate I-3b as a yellow solid. LCMS (Method 2) m/z 281.2 [M+H]⁺ (ES⁺); at 1.60 min. ¹H NMR (500 MHz, DMSO-d6) δ 7.25-7.14 (m, 1H), 5.20 (d, J=5.7 Hz, 1H), 4.21 (t, J=6.3 Hz, 1H), 3.07 (s, 6H), 2.54 (s, 1H), 2.09 (s, 3H), 1.77 (t, J=9.0 Hz, 1H), 1.68-1.56 (m, 1H), 1.38 (s, 9H).

Step 2: To a solution of urea (0.2 g, 4 mmol) in EtOH (10 ml) was added a solution of sodium ethoxide (2 ml, 21% w/w in EtOH, 4 mmol). tert-Butyl (E)-2-((dimethylamino)methylene)-3-oxo-8-azabicyclo[3.2.1]octane-8-carboxylate I-3b (1 g, 4 mmol) in EtOH (10 ml) was added and the reaction mixture was heated to 90° C. for 16 h. Saturated NH₄Cl solution (5 ml) was added and the product was extracted with 10% (0.7 M ammonia/MeOH) in DCM solution (2×35 ml). The product was purified by silica gel chromatography (0-10% (0.7 M ammonia/MeOH) in DCM) to afford tert-butyl 2-hydroxy-6,7,8,9-tetrahydro-5H-5,8-epiminocyclohepta[d]pyrimidine-10-carboxylate I-3c as a white solid. LCMS (Method 2) m/z 278.1 (M+H)⁺ (ES⁺), at 1.3 min. ¹H NMR (500 MHz, DMSO-d6) δ 11.68 (s, 1H), 7.96 (s, 1H), 4.79 (d, J=6.0 Hz, 1H), 4.35 (s, 1H), 2.97 (d, J=17.7 Hz, 1H), 2.16 (s, 1H), 2.02 (dt, J=11.9, 5.6 Hz, 1H), 1.78-1.69 (m, 1H), 1.67 (d, J=17.0 Hz, 1H), 1.36 (s, 9H). 1 proton under DMSO-d6 peak

Step 3: To a solution of tert-butyl (±)-2-oxo-3,5,6,7,8,9-hexahydro-2H-5,8-epiminocyclohepta[d]pyrimidine-10-carboxylate I-3c (600 mg, 1.73 mmol) in DCM (17 ml) was added HCl in 1,4-dioxane (4.33 ml, 4.0 molar, 17.3 mmol) and the mixture was stirred at RT for 16 h. The precipitate was filtered and washed with DCM (2×10 ml). The collected solid was dried in vacuo for 2 h to afford (±)-3,5,6,7,8,9-hexahydro-2H-5,8-epiminocyclohepta[d]pyrimidin-2-one hydrochloride I-3d as a brown solid. The material was used without further purification.

### Intermediate 5 (I-5)

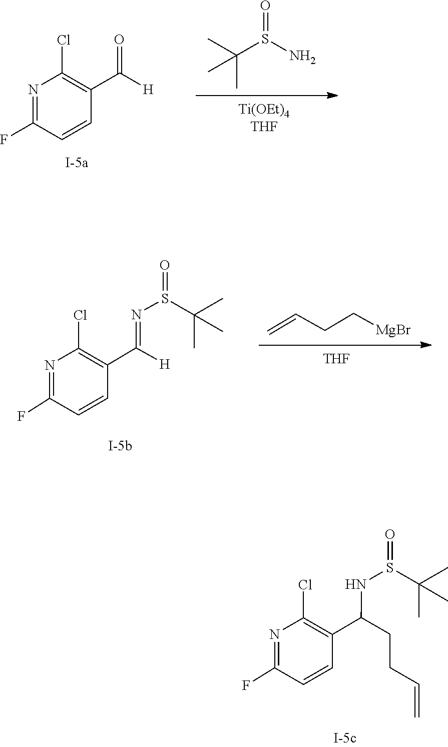

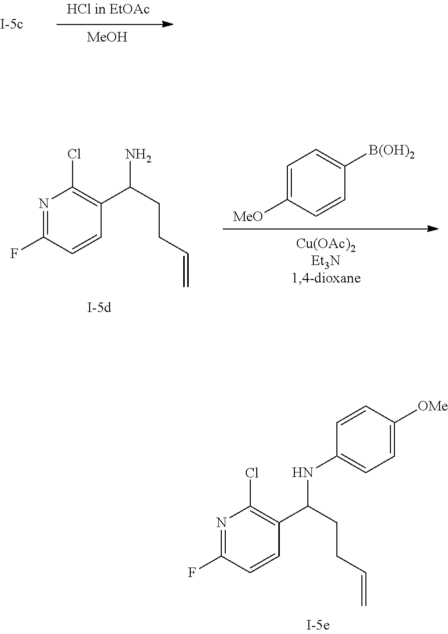 [SMILES: "MeOH" ; C=CCCC(N)c1ccc(F)nc1Cl ; COc1ccc(B(O)O)cc1 ; C=CCCC(Nc1ccc(OC)cc1)c1ccc(F)nc1Cl]

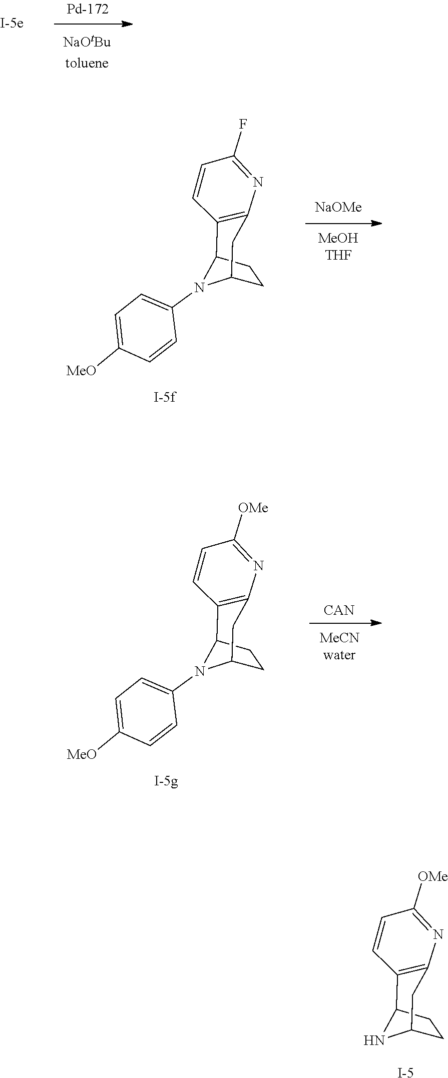

The following scheme makes use of synthetic methodology described in Schultz and Wolfe, Organic Letters, 2011, 13 (11), 2962-2965

Step 1: To a solution of 2-chloro-6-fluoronicotinaldehyde (1-5a) (9.50 g, 59.6 mmol) in THF (57 ml) was added Ti(OEt)₄ (24.7 ml, 119 mmol) in one portion at RT under N₂, the reaction mixture was stirred at RT for 5 min, then tert-butanesulfinamide (7.94 g, 65.5 mmol) was added in one portion. The resulting mixture was stirred at RT for 16 h. The reaction mixture was poured onto the water (50 ml) at 0° C. The reaction mixture was filtered. The filter cake was combined and washed with EtOAc (3×10 ml) and then the filtrate was extracted with EtOAc (3×30 ml). The combined organics were concentrated in vacuo. The product was purified by silica gel chromatography (1%-100% EtOAc/petroleum ether) to give (E)-N-((2-chloro-6-fluoropyridin-3-yl)methylene)-2-methylpropane-2-sulfinamide (I5b) as a white solid. ¹H NMR (400 MHz, CDCl₃) δ 8.93 (s, 1H), 8.48 (t, J=8.4 Hz, 1H), 7.02-6.99 (m, 1H), 1.28 (m, 9H).

Step 2: To a solution of (E)-N-((2-chloro-6-fluoropyridin-3-yl)methylene)-2-methylpropane-2-sulfinamide (I5b) (13.6 g, 51.8 mmol) in THF (79 ml) was added a solution of 3-butenylmagnesium bromide (0.5 M, 11.4 ml) at 0° C. The mixture was warmed to RT for 1 h. Water (50 ml) was added at 0° C., and the product was extracted with EtOAc (3×30 ml). The combined organics were washed with brine and dried over Na₂SO₄ and concentrated in vacuo. The product was purified by silica gel chromatography (1%-100% EtOAc/petroleum ether) to give N-(1-(2-chloro-6-fluoropyridin-3-yl)pent-4-en-1-yl)-2-methylpropane-2-sulfinamide (I5c) as a yellow oil. ¹H NMR: (400 MHz, CDCl₃) δ 7.82-7.87 (m, 1H), 6.87-6.90 (m, 1H), 5.76 (t, J=10.4 Hz, 1H), 5.01-5.05 (m, 2H), 4.98-4.84 (m, 1H), 5.82-3.79 (m, 1H), 2.20-2.05 (m, 2H), 1.94-1.88 (m, 2H) 1.16-1.22 (m, 9H)

Step 3: To a solution of N-(1-(2-chloro-6-fluoropyridin-3-yl)pent-4-en-1-yl)-2-methylpropane-2-sulfinamide (I5c) (15.0 g, 47.1 mmol) in MeOH (104 ml) was added a solution of HCl in EtOAc (4 M, 33.0 ml) at 0° C. The mixture was warmed to RT for 1 hr. The mixture was concentrated in vacuo, the residue was triturated with MTBE (20 ml) at RT for 30 min. The mixture was filtered. The filter cake was combined and washed with MTBE (2×10 ml) and concentrated in vacuo to give a yellow solid. The yellow solid was dissolved in water (15 ml). The mixture was basified to pH>11 with NaOH solution at 0° C. and the product was extracted with DCM (6×15 ml). The combined organics were washed with brine, dried over Na₂SO₄, filtered and concentrated in vacuo. The product was purified by silica gel chromatography (1%-100% EtOAc/petroleum ether). To give 1-(2-chloro-6-fluoropyridin-3-yl)pent-4-en-1-amine (I5d) was as a yellow liquid. ¹H NMR (400 MHz, CDCl₃) δ 8.00 (t, J=8.4 Hz, 1H), 6.87-6.90 (m, 1H), 5.72-5.83 (m 1H), 5.96-5.05 (m, 2H), 4.36 (t, J=5.6 Hz, 1H) 2.09-2.15 (m, 2H), 1.65-1.69 (m, 2H), 1.48 (s, 2H).

Step 4: To a solution 1-(2-chloro-6-fluoropyridin-3-yl)pent-4-en-1-amine (I5d) (5.52 g, 23.4 mmol) and 4-methoxyphenylboronic acid (7.11 g, 46.8 mmol) in 1,4-dioxane (300 ml) was added Cu(OAc)₂ (10.6 g, 58.5 mmol), 4 Å molecular sieves (10.0 g) and Et₃N (4.23 ml, 30.4 mmol) at RT. The mixture was warmed to 35° C. for 16 h. The reaction mixture was filtered. The filter cakes were washed with EtOAc (3×10 ml), The filtrate was concentrated in vacuo. The product was purified by silica gel chromatography (1-100% EtOAc/petroleum ether) to give N-(1-(2-chloro-6-fluoropyridin-3-yl)pent-4-en-1-yl)-4-methoxyaniline (I-5e) as a yellow oil. ¹H NMR (400 MHz, CDCl₃) δ 7.88 (t, J=8.0 Hz, 1H), 6.78-6.83 (m, 1H), 6.70 (t, J=4.4 Hz, 2H), 6.40 (d, J=4.4 Hz, 2H), 5.80-5.87 (m, 1H), 5.01-5.05 (m, 2H), 3.70 (s, 3H), 2.19-2.31 (m, 2H), 1.77-1.92 (m, 2H), 1.43 (s, 1H) (1 exchangeable NH not observed)

Step 5: To a solution of N-(1-(2-chloro-6-fluoropyridin-3-yl)pent-4-en-1-yl)-4-methoxyaniline (I-5e) (3.00 g, 9.35 mmol) in toluene (60 ml) was added NaO^{t}Bu (1.35 g, 14.0 mmol) and Pd-172 (567 mg, 935 umol) at 20° C. The mixture was stirred at 110° C. for 16 h. The reaction mixture was concentrated in vacuo. The product was purified by silica gel chromatography (EtOAc/petroleum ether 1%-100%) to give (±)-2-fluoro-10-(4-methoxyphenyl)-6,7,8,9-tetrahydro-5H-5,8-epiminocyclohepta[b]pyridine (1-5f) as a yellow solid. ¹H NMR (400 MHz, CDCl3) δ 7.55 (t, J=8.0 Hz, 1H), 6.68-6.77 (m, 5H), 4.72 (d, J=2.4 Hz, 1H), 4.52 (d, J=5.6 Hz, 1H), 3.71 (s, 3H), 3.28 (dd, J=8.8, 4.4 Hz, 1H), 2.53 (d, J=8.8 Hz, 1H), 2.42-2.56 (m, 3H), 1.82-1.96 (m, 2H).

Step 6: To a solution of (±)-2-fluoro-10-(4-methoxyphenyl)-6,7,8,9-tetrahydro-5H-5,8-epiminocyclohepta[b]pyridine (1-5f) (113 mg, 397 μmol) in MeOH (4 ml) was added a solution of sodium methoxide in methanol (110 μl, 5.4 molar, 596 μmol). The resultant mixture was heated at 65° C. for 20 h. THF (1 ml) was added followed by a further portion of sodium methoxide (110 μl, 5.4 molar, 596 μmol). The reaction was stirred at 65° C. for a further 24 h. The reaction was cooled. The solid was filtered off and washed with MeOH (2×5 ml) and the solid was dried in vacuo to give (±)-2-methoxy-10-(4-methoxyphenyl)-6,7,8,9-tetrahydro-5H-5,8-epiminocyclohepta[b]pyridine (I-5g) as a white solid. LC-MS (Method 1) m/z 297.1 (M+H)⁺ (ES⁺); at 1.68 min

Step 7: To a solution of (±)-2-methoxy-10-(4-methoxyphenyl)-6,7,8,9-tetrahydro-5H-5,8-epiminocyclohepta[b]pyridine (I-5g) (424 mg, 1.43 mmol) in MeCN (21 ml) at 0° C. was added a solution of CAN (2.51 g, 4.58 mmol) in water (21 ml) dropwise. After the addition the reaction was stirred at 0° C. for 1 h. 2 M NaOH (25 ml) was added and the mixture was filtered. DCM was added and the precipitate was washed with DCM and water. The layers were separated and the aqueous was extracted with DCM (3×30 ml). The combined organics were dried with MgSO₄ to give (±)-2-methoxy-6,7,8,9-tetrahydro-5H-5,8-epiminocyclohepta[b]pyridine I-5 as an off white solid. ¹H NMR (400 MHz, DMSO-d6) δ 7.32 (d, J=8.2 Hz, 1H), 6.53-6.44 (m, 1H), 4.08 (d, J=5.3 Hz, 1H), 3.76 (s, 4H), 3.01 (dd, J=17.4, 5.1 Hz, 1H), 2.44 (d, J=17.3 Hz, 1H), 1.97-1.78 (m, 2H), 1.73-1.63 (m, 1H), 1.53-1.42 (m, 1H). (NH proton not observed)

### Intermediate 6 (I-6)

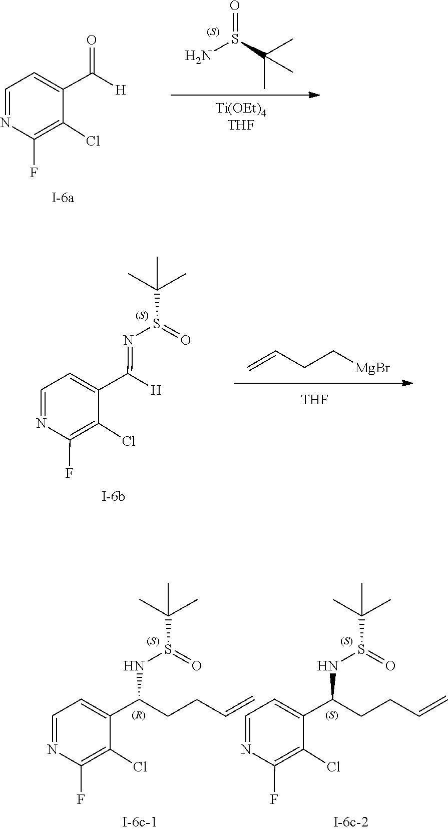

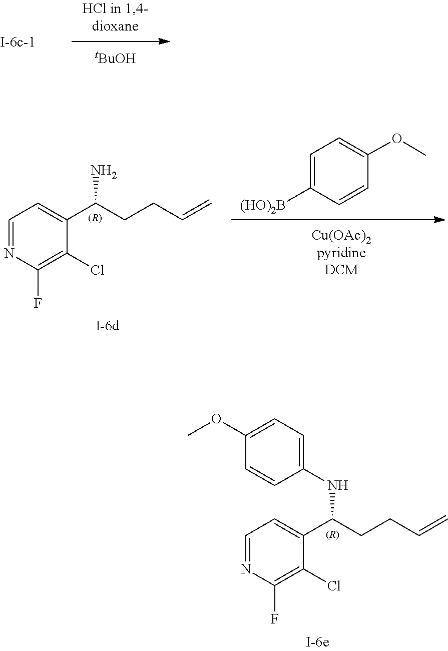

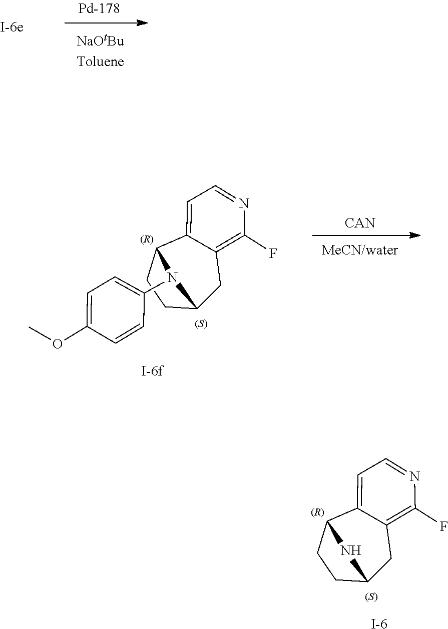

Step 1: To a solution of 3-chloro-2-fluoroisonicotinaldehyde I-6a (1.00 g, 6.27 mmol) in THF (17 ml) was added titanium (IV) ethoxide (2.63 ml, 12.5 mmol) in one portion at RT. The mixture was stirred at RT for 5 min before (S)-tert-butylsulfinamide (760 mg, 6.27 mmol) was added in one portion. The resulting mixture was stirred at RT for 16 h. Brine (30 ml) was added, and the mixture stirred for 10 min then filtered through celite. The filtrate was extracted with EtOAc (2×20 ml). The combined organics were dried over MgSO₄ and concentrated in vacuo to give (S)—N-((3-chloro-2-fluoropyridin-4-yl)methylene)-2-methylpropane-2-sulfinamide (1-6b) as a pale yellow solid. LCMS (Method 1) m/z 227.5 (M−Cl)⁺ (ES⁺), at 1.36 min. ¹H NMR (500 MHz, DMSO-d6) δ 8.81 (s, 1H), 8.33 (dt, J=5.1, 0.8 Hz, 1H), 7.90 (d, J=5.1 Hz, 1H), 1.22 (s, 9H).

Step 2: To a solution of (S)—N-((3-chloro-2-fluoropyridin-4-yl)methylene)-2-methylpropane-2-sulfinamide (1.38 g, 5.26 mmol) (1-6b) in THF (22 ml) was added but-3-en-1-yl magnesium bromide (31.6 ml, 0.5 M, 15.8 mmol) dropwise at −78° C. The mixture was warmed to RT slowly and stirred for 72 h. Saturated NH₄Cl solution (10 ml) was added and the product was extracted with EtOAc (3×10 ml). The combined organics were dried with MgSO₄ and concentrated in vacuo. The product was purified by silica gel chromatography (0-100% EtOAc/isohexane) to give (S)—N—(I-1-(3-chloro-2-fluoropyridin-4-yl)pent-4-en-1-yl)-2-methylpropane-2-sulfinamide (I-6c-1) as a pale yellow solid. LCMS (Method 1) m/z 316.7, 319.1 (M−H)⁻ (ES⁻), at 1.39 min. ¹H NMR (500 MHz, DMSO-d6) δ 8.21 (dd, J=5.2, 0.8 Hz, 1H), 7.53 (d, J=5.2 Hz, 1H), 5.90 (d, J=7.3 Hz, 1H), 5.81 (dddd, J=17.3, 10.2, 7.1, 6.1 Hz, 1H), 5.10 (dq, J=17.2, 1.7 Hz, 1H), 5.02 (ddt, J=10.2, 2.3, 1.2 Hz, 1H), 4.66 (ddd, J=8.6, 7.3, 5.3 Hz, 1H), 2.27-2.08 (m, 2H), 1.0-7-1.87 (m, 1H), 1.0-0-1.70 (m, 1H), 1.07 (s, 9H).

(S)—N—((S)-1-(3-chloro-2-fluoropyridin-4-yl)pent-4-en-1-yl)-2-methylpropane-2-sulfinamide (I-6c-2) was also obtained from the purification

Step 3: To a solution of (S)-/(R)-1-(3-chloro-2-fluoropyridin-4-yl)pent-4-en-1-yl)-2-methylpropane-2-sulfinamide (1-6c) (714 mg, 2.015 mmol) in ^{t}BuOH (7.2 ml) was added a solution of HCl in 1,4-dioxane (3.0 ml, 4 M, 12.09 mmol) at RT and stirred for 2.5 h. The reaction mixture was quenched with saturated aqueous NaHCO₃ solution (100 ml) and extracted with DCM (3×30 ml). The combined organics were dried over MgSO₄ and concentrated in vacuo tolve (R)-1-(3-chloro-2-fluoropyridin-4-yl)pent-4-en-1-amine (1-6d) as a pale yellow liquid LCMS (Method 1): m/z 215.4, 217.3 (M+H)⁺ (ES⁺); at 1.17 min, ¹H NMR (500 MHz, DMSO-d6) δ 8.16 (dd, J=5.1, 0.9 Hz, 1H), 7.62 (d, J=5.1 Hz, 1H), 5.80 (ddt, J=16.9, 10.2, 6.6 Hz, 1H), 5.02 (dq, J=17.4, 1.8 Hz, 1H), 4.96 (ddt, J=10.2, 2.3, 1.3 Hz, 1H), 4.22 (dd, J=8.1, 5.1 Hz, 1H), 2.23-2.01 (m, 2H), 1.76-1.50 (m, 2H), 2 NH protons not observed.

Step 4: To a sollon of (R)-1-(3-chloro-2-fluoropyridin-4-yl)pent-4-en-1-amine (1-6d) (644.3 mg, 3.001 mmol), (4-methoxyphenyl)boronic acid (1.37 g, 9.0 mmol), and Cu(OAc)₂ (820 mg, 4.50 mmol) in DCM (100 ml) was added pyridine (1.2 ml, 15.0 mmol) dropwise. The mixture was stirred at RT, open to air for 16 h. 2 M NaOH aqueous solution (20 ml) was added followed by water (20 ml) and the mixture extracted with DCM (3×20 ml). The combined organics were dried over MgSO₄ and concentrated in vacuo. The product was purified by silica gel chromatography (0%-100% DCM/isohexal to give (R)—N-(1-(3-chloro-2-fluoropyridin-4-yl)pent-4-en-1-yl)-4-methoxyaniline (I-6e) as a yellow oil. LCMS (Method 1): m/z 321.1, 323.4 (M+H)⁺ (ES⁺); at 1.73 min, ¹H NMR (500 MHz, DMSO-d6) δ 8.10 (d, J=5.1 Hz, 1H), 7.41 (d, J=5.1 Hz, 1H), 6.65 (d, J=8.9 Hz, 2H), 6.38 (d, J=8.9 Hz, 2H), 6.09 (d, J=8.3 Hz, 1H), 5.84 (ddt, J=17.0, 10.2, 6.6 Hz, 1H), 5.10-4.88 (m, 2H), 4.68 (td, J=8.5, 4.7 Hz, 1H), 3.57 (s, 3H), 2.34-2.24 (m, 1H), 2.23-2.12 (m, 1H), 1.87-1.69 (m, 2H).

Step 5: A three-neck flask was charged with Pd-178 (7.4 mg, 15.6 μmol) and sodium tert-butoxide (22.5 mg, 234 μmol) and purged with N₂·I solution of (R)—N-(1-(3-chloro-2-fluoropyridin-4-yl)pent-4-en-1-yl)-4-methoxyaniline (I-6e) (50.0 mg, 156 μmol) in toluene (1 ml) was added dropwise. The resulting mixture was heated to 95° C. for 1.5 h. The reaction mixture was cooled to RT and filtered through celite, washing with EtOAc (3×20 ml). The filtrate was concentrated in vacuo. The product was purified by silica gel chromatography (0%-50% EtOAc/heptane) to give (5R,8S)-1-fluoro-10-(4-methoxyphenyl)-6,7,8,9-tetrahydro-5H-5,8-epiminocyclohepta[c]pyridine (1-6f) as a white solid. LCMS (Method 1) m/z 285.3 (M+H)⁺ (ES⁺), at 1.35 min. ¹H NMR (500 MHz, DMSO-d6) δ 7.95 (d, J=4.9 Hz, 1H), 7.29 (dd, J=5.0, 1.7 Hz, 1H), 6.80 (d, J=9.1 Hz, 2H), 6.71 (d, J=9.1 Hz, 2H), 4.92 (d, J=5.6 Hz, 1H), 4.56 (t, J=5.9 Hz, 1H), 3.61 (s, 3H), 2.92 (dd, J=17.6, 4.9 Hz, 1H), 2.35 (d, J=17.5 Hz, 1H), 2.32-2.19 (m, 2H), 1.91-1.82 (m, 1H), 1.81-1.74 (m, 1H).

Step 6: A solution of (5R,8S)-1-fluoro-10-(4-methoxyphenyl)-6,7,8,9-tetrahydro-5H-5,8-epiminocyclohepta[c]pyridine (1-6f) (69 mg, 232 μmol) in MeCN (3.2 ml) was cooled to 0° C. before a solution of CAN (381 mg, 696 μmol) in water (3.2 ml) was added dropwise. After the addition was complete, the reaction was stirred for 1 h at 0° C. 2 M aqueous NaOH (5 ml) and water (5 ml) were added, and the mixture extracted with DCM (3×10 ml). The combined organics were dried over MgSO₄ and concentrated in vacuo to give (5R,8S)-1-fluoro-6,7,8,9-tetrahydro-5H-5,8-epiminocyclohepta[c]pyridine (1-6) as a pale pink solid. LCMS (Method 1): m/z 179.2 (M+H)⁺ (ES⁺); at 0.69 min, ¹H NMR (500 MHz, DMSO-d6) δ 7.91 (dd, J=5.0, 0.9 Hz, 1H), 7.04 (dd, J=5.0, 1.9 Hz, 1H), 4.17 (d, J=5.3 Hz, 1H), 3.78 (t, J=6.0 Hz, 1H), 2.85 (dd, J=17.1, 5.2 Hz, 1H), 2.74 (s, 1H), 2.38 (d, J=17.0 Hz, 1H), 2.01-1.85 (m, 2H), 1.73 (t, J=9.1 Hz, 1H), 1.51 (dt, J=9.4, 5.4 Hz, 1H).

### Intermediate 7 (I-7)—Route 1

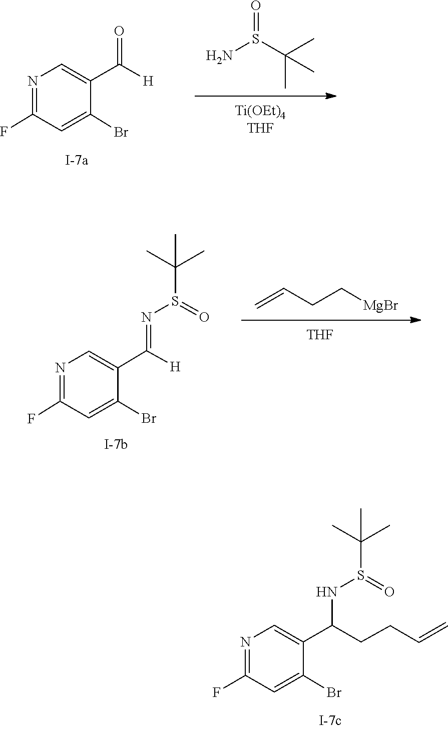

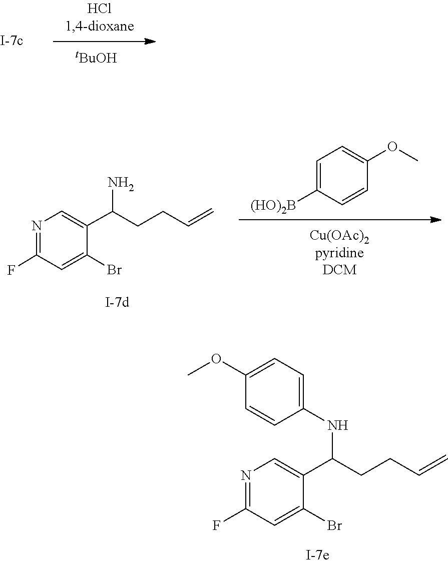 [SMILES: "tBuOH" ; C=CCCC(N)c1cnc(F)cc1Br ; COc1ccc(B(O)O)cc1 ; C=CCCC(Nc1ccc(OC)cc1)c1cnc(F)cc1Br]

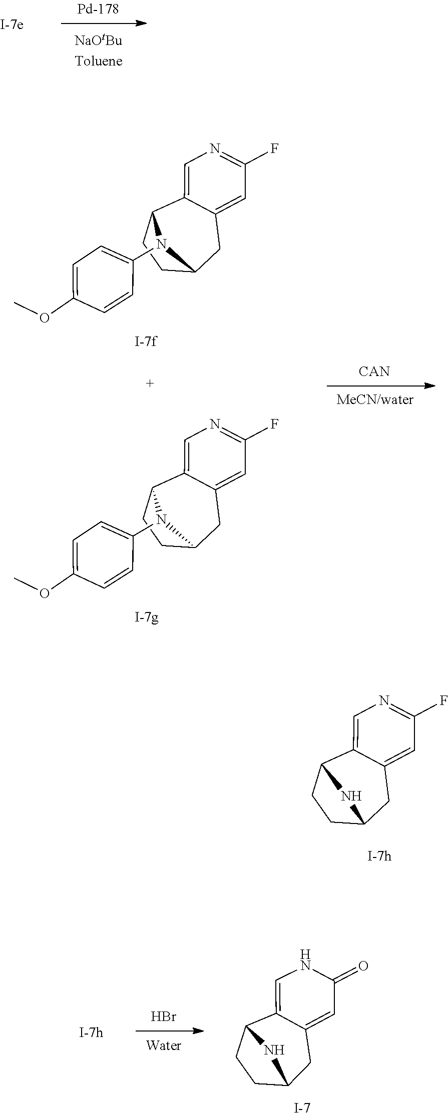

Step 1: (S)—N-((4-Bromo-6-fluoropyridin-3-yl)methylene)-2-methylpropane-2-sulfinamide I-7b was synthesised from 4-bromo-6-fluoronicotinaldehyde I-7a using a procedure essentially the same as for I-6b. LCMS (Method 2): m/z 307.0, 308.9 (M+H)⁺ (ES⁺); at 2.05 min, ¹H NMR (500 MHz, DMSO-d6) δ 8.81 (s, 1H), 8.73 (s, 1H), 7.88 (d, J=2.4 Hz, 1H), 1.21 (s, 9H).

Step 2: To a solution of ((S)—N-((4-bromo-6-fluoropyridin-3-yl)methylene)-2-methylpropane-2-sulfinamide I-7b (7.00 g, 22.8 mmol) in THF (114 ml) was added but-3-en-1-yl magnesium bromide (2.54 M, 17.9 ml, 45.6 mmol) dropwise at −78° C. The mixture was warmed to RT slowly and stirred for 16 h. Saturated NH₄Cl solution (10 ml) was added and the product was extracted with EtOAc (2×10 ml). The combined organics were dried with MgSO₄ and concentrated in vacuo. The product was purified by silica gel chromatography (0-10% IPA/isohexane) to give a 2:1 mixture of the diastereomers of (±)—N-(1-(4-bromo-6-fluoropyridin-3-yl)pent-4-en-1-yl)-2-methylpropane-2-sulfinamide I-7c as a pale yellow oil. LCMS (Method 1) m/z 363.3, 365.4 (M+H)⁺ (ES⁺), at 1.39 and 1.42 min.

Major diastereomer: 1H NMR (500 MHz, DMSO-d6) δ 8.43 (s, 1H), 7.60 (d, J=2.6 Hz, 1H), 6.00 (d, J=9.5 Hz, 1H), 5.82 (ddt, J=16.7, 10.3, 6.6 Hz, 1H), 5.12-4.97 (m, 2H), 4.64-4.50 (m, 1H), 2.26-2.14 (m, 1H), 2.16-2.05 (m, 1H), 1.94-1.78 (m, 1H), 1.78-1.66 (m, 1H), 1.13 (s, 9H).

Minor diastereomer: 1H NMR (500 MHz, DMSO-d6) δ 8.33 (s, 1H), 7.61 (d, J=2.5 Hz, 1H), 5.82 (ddt, J=16.7, 10.3, 6.6 Hz, 1H), 5.76 (d, J=7.0 Hz, 1H), 5.12-4.97 (m, 2H), 4.64-4.50 (m, 1H), 2.26-2.14 (m, 1H), 2.16-2.05 (m, 1H), 2.03-1.92 (m, 1H), 1.94-1.78 (m, 1H), 1.07 (s, 9H).

Step 3: (±)-1-(4-Bromo-6-fluoropyridin-3-yl)pent-4-en-1-amine I-7d was synthesised from (±)—N-(1-(4-bromo-6-fluoropyridin-3-yl)pent-4-en-1-yl)-2-methylpropane-2-sulfinamide I-7c using a procedure essentially the same as for I-6d. LCMS (Method 1) m/z 259.2, 261.2 (M+H)⁺ (ES⁺), at 1.21 min. ¹H NMR (500 MHz, DMSO-d6) δ 8.41 (s, 1H), 7.55 (d, J=2.7 Hz, 1H), 5.82 (ddt, J=16.9, 10.2, 6.6 Hz, 1H), 5.03 (dq, J=17.2, 1.8 Hz, 1H), 4.95 (ddt, J=10.2, 2.3, 1.3 Hz, 1H), 4.12 (dd, J=8.2, 4.9 Hz, 1H), 2.25-1.98 (m, 4H), 1.75-1.64 (m, 1H), 1.64-1.54 (m, 1H).

Step 4: (±)—N-(1-(4-Bromo-6-fluoropyridin-3-yl)pent-4-en-1-yl)-4-methoxyaniline I-7e was synthesised from (±)-1-(4-bromo-6-fluoropyridin-3-yl)pent-4-en-1-amine I-7d using a procedure essentially the same as for I-6e. LCMS (Method 1) m/z 365.0, 367.1 (M+H)⁺ (ES⁺), at 1.76 min. ¹H NMR (500 MHz, DMSO-d6) δ 8.21 (s, 1H), 7.61 (d, J=2.5 Hz, 1H), 6.66 (d, J=8.9 Hz, 2H), 6.40 (d, J=9.0 Hz, 2H), 6.04 (d, J=8.3 Hz, 1H), 5.86 (ddt, J=17.0, 10.2, 6.6 Hz, 1H), 5.08-4.90 (m, 2H), 4.59 (td, J=8.5, 4.8 Hz, 1H), 3.58 (s, 3H), 2.35-2.26 (m, 1H), 2.23-2.13 (m, 1H), 1.92-1.71 (m, 2H).

Step 5: A three-neck flask was charged with Pd-178 (0.29 g, 0.61 mmol) and NaO^{t}Bu (0.88 g, 9.2 mmol) and purged with N₂. A solution of N-(1-(4-bromo-6-fluoropyridin-3-yl)pent-4-en-1-yl)-4-methoxyaniline I-7e (2.3 g, 6.1 mmol) in toluene (66 ml) was added dropwise. The resulting mixture was heated to 95° C. for 2 h. The reaction mixture was cooled to RT and filtered through celite, washing the solid with EtOAc (150 ml). The filtrate was concentrated in vacuo. The product was purified by silica gel chromatography (0%-50% EtOAc/isohexane) to give a mixture of enantiomers which were dissolved to 30 mg/ml in DCM:methanol (1:4) and was then separated by chiral SFC on a Waters prep 15 with UV detection at 210 nm, 40° C., 100 bar on a Lux C₃ column (21.2 mm×250 mm, 5 μm particle size), flow rate 50 ml/min using 40% methanol to give (6S,9R)-3-fluoro-10-(4-methoxyphenyl)-6,7,8,9-tetrahydro-5H-6,9-epiminocyclohepta[c]pyridine (I-7f) as a pale yellow solid. LCMS (Method 1) m/z 285.3 (M+H)⁺ (ES⁺), at 1.35 min. ¹H NMR (500 MHz, DMSO-d6) δ 8.10 (s, 1H), 6.79 (t, J=9.7 Hz, 3H), 6.70 (d, J=9.1 Hz, 2H), 4.95 (d, J=5.5 Hz, 1H), 4.47 (t, J=6.0 Hz, 1H), 3.61 (s, 3H), 3.11 (dd, J=18.2, 4.9 Hz, 1H), 2.31-2.19 (m, 2H), 1.88-1.55 (m, 2H), 1 proton obscured by residual DMSO peak.

and (6R,9S)-3-fluoro-10-(4-methoxyphenyl)-6,7,8,9-tetrahydro-5H-6,9-epiminocyclohepta[c]pyridine (I-7g) as a pale yellow solid. LCMS (Method 1) m/z 285.3 (M+H)⁺ (ES⁺), at 1.35 min. ¹H NMR (500 MHz, DMSO-d6) δ 8.10 (s, 1H), 6.79 (t, J=9.7 Hz, 3H), 6.70 (d, J=9.1 Hz, 2H), 4.95 (d, J=5.5 Hz, 1H), 4.47 (t, J=6.0 Hz, 1H), 3.61 (s, 3H), 3.11 (dd, J=18.2, 4.9 Hz, 1H), 2.31-2.19 (m, 2H), 1.88-1.55 (m, 2H), 1 proton obscured by residual DMSO peak.

Step 6: (6S,9R)-3-Fluoro-6,7,8,9-tetrahydro-5H-6,9-epiminocyclohepta[c]pyridine (I-7 h) was synthesised from (6S,9R)-3-fluoro-10-(4-methoxyphenyl)-6,7,8,9-tetrahydro-5H-6,9-epiminocyclohepta[c]pyridine (I-17f) using a procedure essentially the same as for I-6. LCMS (Method 1) m/z 179.2 (M+H)⁺ (ES⁺), at 0.71 min.

Step 7: (6S,9R)-3-Fluoro-6,7,8,9-tetrahydro-5H-6,9-epiminocyclohepta[c]pyridine I-6 (300 mg, 1.68 mmol) was dissolved in HBr (1.90 ml, 48% w/w in water, 16.8 mmol) and was heated at 100° C. for 18 h. The reaction mixture was cooled to RT, diluted with MeOH, loaded onto a SCX cartridge (20 g) and the product was eluted with ammonia in MeOH solution (0.7 M) to give (6S,9R)-2,5,6,7,8,9-hexahydro-3H-6,9-epiminocyclohepta[c]pyridin-3-one (I-7) as an off white solid. LCMS (Method 1) m/z 176.9 (M+H)⁺ (ES⁺), at 0.41 min,

### Intermediate 7 (I-7)—Route 2

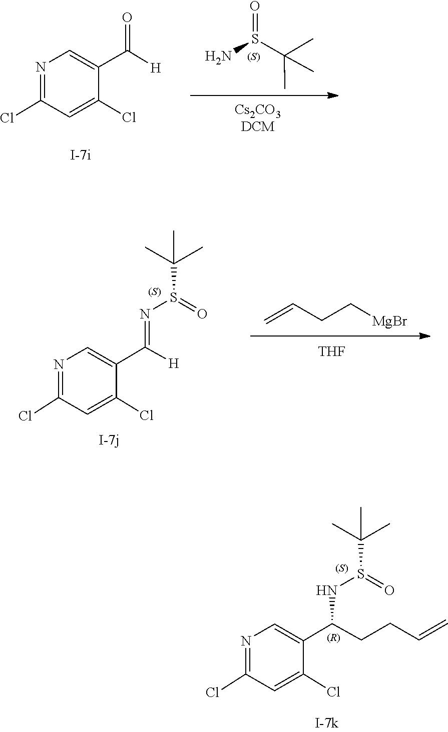

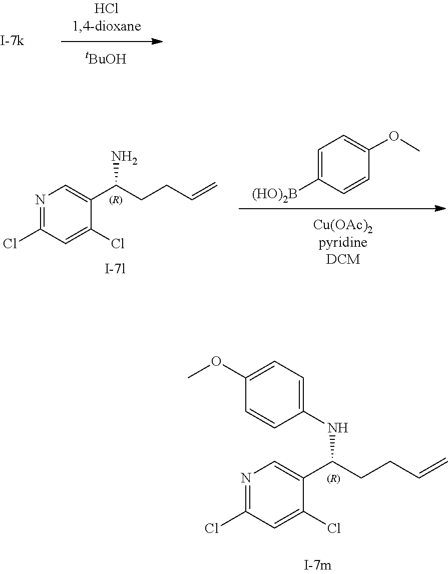

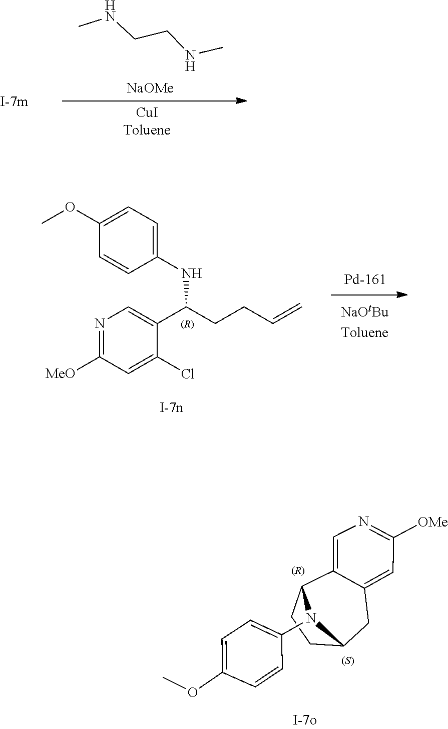

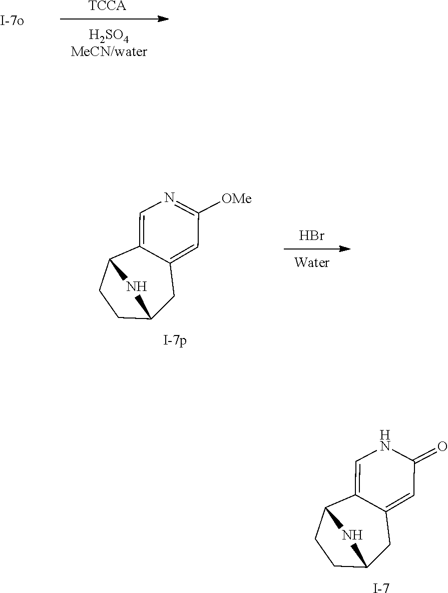

Step 1: To a solution of 4,6-dichloronicotinaldehyde I-7i (17.3 g, 88.5 mmol) and (S)-2-methylpropane-2-sulfinamide (10.7 g, 88.5 mmol) in DCM (150 ml) was added cesium carbonate (28.8 g, 88.5 mmol). The resultant mixture was stirred at RT for 16 h. The material was filtered and the solid residue was washed with DCM (150 ml). The solvent was evaporated to give (S)—N-((4,6-dichloropyridin-3-yl)methylene)-2-methylpropane-2-sulfinamide I-7j as an off white solid. ¹H NMR (400 MHz, DMSO-d6) δ 8.97 (s, 1H), 8.76 (s, 1H), 8.04 (s, 1H), 1.21 (s, 9H).

Step 2: A solution of but-3-en-1-ylmagnesium bromide (0.5 M in THF) (60.9 ml, 30.4 mmol) was slowly added to a solution of (S)—N-((4,6-dichloropyridin-3-yl)methylene)-2-methylpropane-2-sulfinamide I-7j (5.00 g, 1 Eq, 17.9 mmol) in THF (100 mL) at −78° C. The reaction mixture was allowed to slowly warm up to RT over 16 h. The reaction was cooled to 0-10° C. and saturated aqueous NH₄Cl (100 ml) was added and stirred for 5 min. The layers were separated and the aqueous phase was extracted with EtOAc (2×250 ml). The combined organics were washed with brine (50 ml), dried with Na₂SO₄ and concentrated in vacuo. The product was purified by chromatography on silica gel (0-30% MTBE in DCM) to afford (S)—N—((R)-1-(4,6-dichloropyridin-3-yl)pent-4-en-1-yl)-2-methylpropane-2-sulfinamide I-7k as a clear yellow oil. ¹H NMR (400 MHz, DMSO-d6) δ8.53 (s, 1H), 7.77 (s, 1H), 5.85-5.75 (m, 2H), 5.07 (dq, J=17.2, 1.6 Hz, 1H), 5.00 (ddt, J=10.3, 2.3, 1.3 Hz, 1H), 4.66-4.56 (m, 1H), 2.20-2.05 (m, 2H), 1.98 (dtd, J=13.9, 8.0, 5.7 Hz, 1H), 1.83 (ddt, J=13.3, 8.7, 6.5 Hz, 1H), 1.07 (s, 9H).

Step 3: A solution of HCl (4 M in dioxane) (41 ml, 0.16 mol) was added to a solution of (S)—N—((R)-1-(4,6-dichloropyridin-3-yl)pent-4-en-1-yl)-2-methylpropane-2-sulfinamide I-7k (11 g, 33 mmol) in ^{t}BuOH (50 ml) and the reaction mixture was stirred at RT for 90 min. The reaction mixture was cooled in an ice bath and water (220 ml) was added and stirred for 10 min. The aqueous was extracted with MTBE (3×30 ml). The organic layer was extracted with water (2×30 ml). The aqueous was basified using saturated aqueous NaHCO₃ solution and more solid NaHCO₃ and the mixture was stirred for 15 min. The product was extracted with MTBE (3×100 ml). The combined organics were washed with water (500 ml), dried with Na₂SO₄ and concentrated in vacuo to give (R)-1-(4,6-dichloropyridin-3-yl)pent-4-en-1-amine I-71 as an orange oil. ¹H NMR (400 MHz, DMSO-d6) δ 8.60 (s, 1H), 7.69 (s, 1H), 5.80 (ddt, J=16.9, 10.1, 6.6 Hz, 1H), 5.01 (dq, J=17.2, 1.7 Hz, 1H), 4.94 (ddt, J=10.2, 2.3, 1.3 Hz, 1H), 4.15 (dd, J=8.0, 5.2 Hz, 1H), 2.17-2.01 (m, 4H), 1.74-1.54 (m, 2H).

Step 4: To a solution of (R)-1-(4,6-dichloropyridin-3-yl)pent-4-en-1-amine I-71 (7.04 g, 30.5 mmol) in DCM (70 ml) was added (4-methoxyphenyl)boronic acid (13.9 g, 91.4 mmol), copper (II) acetate (6.09 g, 33.5 mmol) and Et₃N (21.2 ml, 152 mmol). The resultant mixture was stirred at RT for 20 h. A further portion of copper (II) acetate (2.21 g, 12.2 mmol), (4-methoxyphenyl)boronic acid (5.55 g, 36.6 mmol) and Et₃N (5.09 ml, 36.6 mmol) was added and the mixture was stirred for a further 20 h. 1 M HCl (200 ml) was added and the layers were separated. Ammonium hydroxide solution (28% w/v, 100 and 200 ml) was added to the organic and aqueous layers respectively. The layers were separated and the aqueous was extracted with DCM (100 ml). The combined organics were washed with water (100 ml) and dried with MgSO₄. The product was purified by chromatography on silica gel (0-50% EtOAc/isohexane) to afford (R)—N-(1-(4,6-dichloropyridin-3-yl)pent-4-en-1-yl)-4-methoxyaniline I-7 m as a thick colourless oil. ¹H NMR (400 MHz, DMSO-d6) δ 8.42 (s, 1H), 7.75 (s, 1H), 6.70-6.59 (m, 2H), 6.46-6.34 (m, 2H), 6.01 (d, J=8.5 Hz, 1H), 5.90-5.77 (m, 1H), 5.08-4.94 (m, 2H), 4.64 (td, J=8.6, 4.9 Hz, 1H), 3.58 (s, 3H), 2.27 (ddd, J=12.7, 9.3, 6.1 Hz, 1H), 2.22-2.08 (m, 1H), 1.92-1.72 (m, 2H).

Step 5: To a solution of (R)—N-(1-(4,6-dichloropyridin-3-yl)pent-4-en-1-yl)-4-methoxyaniline I-7m (2.73 g, 8.10 mmol) in toluene (20 ml) was added N,N-dimethylethane-1,2-diamine (86.8 μl, 810 μmol), copper(I) iodide (30.8 mg, 162 μmol) and sodium methoxide (656 mg, 243 μmol). The resultant mixture was heated at 100° C. for 96 h. The reaction mixture was filtered through a pad of celite and the filtrate was concentrated in vacuo. The product was purified by chromatography on silica gel (0-30% MTBE in isohexane) to afford (R)—N-(1-(4-chloro-6-methoxypyridin-3-yl)pent-4-en-1-yl)-4-methoxyaniline I-7n as a thick yellow oil. ¹H NMR (400 MHz, DMSO-d6) δ 8.17 (s, 1H), 6.94 (s, 1H), 6.66-6.62 (m, 2H), 6.43-6.37 (m, 2H), 5.91 (d, J=8.5 Hz, 1H), 5.89-5.78 (m, 1H), 5.07-4.93 (m, 2H), 4.57 (td, J=8.5, 5.1 Hz, 1H), 3.79 (s, 3H), 3.58 (s, 3H), 2.27 (dt, J=14.3, 7.3 Hz, 1H), 2.22-2.07 (m, 1H), 1.80 (dddd, J=20.8, 13.8, 10.1, 5.1 Hz, 2H).

Step 6: Pd-161 (763.6 mg, 1.65 mmol) and NaO^{t}Bu (2.38 g, 24.8 mmol) were placed in a 3-necked RB flask, which was purged under vacuum and backfilled with N₂ (3 times). (R)—N-(1-(4-chloro-6-methoxypyridin-3-yl)pent-4-en-1-yl)-4-methoxyaniline I-7n (5.79 g, 16.52 mmol) was purged under vacuum and backfilled with N₂ (3 times). Toluene (180 ml) was added to amine and the resultant solution was transferred to the 3-necked RB flask. The RB flask was purged under vacuum and backfilled with N₂ (3 times). The resultant mixture was heated at 95° C. for 2 h. The reaction was cooled and filtered through a pad of celite. The filter cake was washed with EtOAc. The filtrate was concentrated in vacuo. The product was purified by chromatography on silica gel (0-50% EtOAc/isohexane) to afford (6S,9R)-3-methoxy-10-(4-methoxyphenyl)-6,7,8,9-tetrahydro-5H-6,9-epiminocyclohepta[c]pyridine I-70 as a light orange solid. ¹H NMR (500 MHz, DMSO-d6) δ 8.01 (s, 1H), 6.82-6.73 (m, 2H), 6.73-6.64 (m, 2H), 6.39 (s, 1H), 4.83 (d, J=5.5 Hz, 1H), 4.43 (m, 1H), 3.74 (s, 3H), 3.61 (s, 3H), 3.04 (dd, J=18.0, 4.9 Hz, 1H), 2.41 (d, J=17.9 Hz, 1H), 2.32-2.14 (m, 2H), 1.84-1.63 (m, 2H).

Step 7: To a solution of (6S,9R)-3-methoxy-10-(4-methoxyphenyl)-6,7,8,9-tetrahydro-5H-6,9-epiminocyclohepta[c]pyridine I-70 (2.00 g, 6.61 mmol) in MeCN (75 ml) and water (75 ml) was added sulfuric acid (6.6 ml, 1 M, 6.61 mmol) followed by trichloroisocyanuric acid (769 mg, 3.31 mmol). The reaction mixture was stirred at RT for 16 h. The mixture was extracted with DCM (3×200 ml). The combined organics were extracted with water (50 ml). The aqueous layer was basified with KOH (3.6 ml, 5 M) and extracted with 10% MeOH in DCM (300 ml). More KOH (1.8 ml, 5 M) was added and the aqueous layer was extracted with 10% MeOH in DCM (100 ml). A further portion of KOH (1.8 ml, 5 M) was added and the aqueous layer was extracted with 10% MeOH in DCM (150 ml). The combined organics were dried over Na₂SO₄ and concentrated in vacuo to give (6S,9R)-3-methoxy-6,7,8,9-tetrahydro-5H-6,9-epiminocyclohepta[c]pyridine I-7p as a brown oil. ¹H NMR (400 MHz, DMSO-d6) δ 7.78 (s, 1H), 6.47 (s, 1H), 4.17-4.11 (m, 1H), 3.76 (s, 3H), 3.66 (td, J=5.2, 2.7 Hz, 1H), 2.95 (ddd, J=17.4, 3.9, 2.4 Hz, 1H), 2.57 (s, 1H), 2.46 (dt, J=17.3, 1.2 Hz, 1H), 1.96-1.81 (m, 2H), 1.72-1.60 (m, 1H), 1.50-1.36 (m, 1H).

Step 8: A solution of (6S,9R)-3-methoxy-6,7,8,9-tetrahydro-5H-6,9-epiminocyclohepta[c]pyridine I-7p (0.99 g, 5.2 mmol) in HBr (48% in water) (8.8 ml, 78 mmol) was heated at reflux for 16 h. The mixture was concentrated in vacuo and the concentrate was diluted with MeOH, loaded onto a SCX cartridge (80 g), the cartridge was washed with MeOH and the product was eluted with NH₃ in MeOH solution (0.7 M). To give (6S,9R)-2,5,6,7,8,9-hexahydro-3H-6,9-epiminocyclohepta[c]pyridin-3-one I-7 as a brown solid. ¹H NMR (400 MHz, DMSO-d6) δ 11.11 (s, 1H), 7.03 (s, 1H), 6.01 (s, 1H), 4.04 (d, J=5.4 Hz, 1H), 3.62 (t, J=5.9 Hz, 1H), 2.83 (dd, J=17.7, 5.1 Hz, 1H), 2.40 (dt, J=17.7, 1.3 Hz, 1H), 1.92-1.78 (m, 2H), 1.68-1.56 (m, 1H), 1.45 (dt, J=9.3, 4.8 Hz, 1H), Exchangeable NH not observed

### Intermediate 8 (I-8)

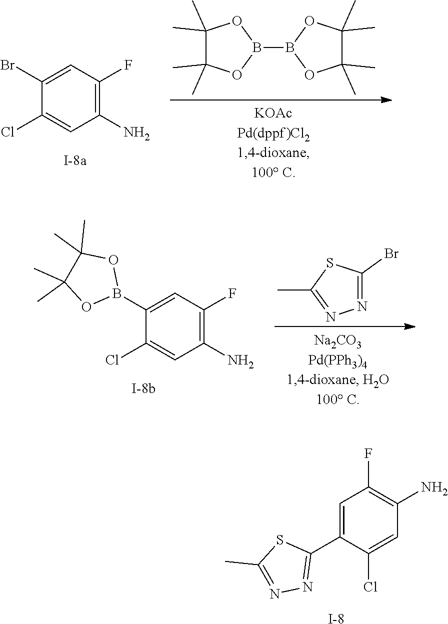

Step 1: To a mixture of 4-bromo-5-chloro-2-fluoroaniline I-8a (300 mg, 1.34 mmol) in 1,4-dioxane (5 ml) was added bis(pinacolato)diboron (509 mg, 2.00 mmol), KOAc (394 mg, 4.00 mmol) and Pd(dppf)Cl₂ (98 mg, 0.13 mmol) under N₂. The reaction mixture was heated at reflux for 1 h then poured into water and the product was extracted with DCM (2×20 ml). The combined organics were washed with brine, dried over Na₂SO₄ and concentrated in vacuo. The product was purified by silica gel chromatography (10% EtOAc/petroleum ether) to give 5-chloro-2-fluoro-4-(4,4,5,5-tetramethyl-1,3,2-dioxaborolan-2-yl)aniline I-8b as a white solid. ¹H NMR: (400 MHz, DMSO-d6) δ 7.14 (d, J=12 Hz, 1H), 6.73 (d, J=7.6 Hz, 1H), 5.86 (s, 2H), 1.25 (s, 12H)

Step 2: To a mixture of 5-chloro-2-fluoro-4-(4,4,5,5-tetramethyl-1,3,2-dioxaborolan-2-yl) aniline I-8b (314 mg, 1.16 mmol) in a mixture of 1,4-dioxane (5 ml) and water (1 ml) were added 2-bromo-5-methyl-1,3,4-thiadiazole (138 mg, 0.77 mmol), Na₂CO₃ (245 mg, 2.31 mmol) and Pd(PPh₃)₄ (89 mg, 0.08 mmol) under N₂. The reaction was heated at 100° C. for 16 h, then poured into water and extracted with DCM (2×20 ml). The combined organics were washed with brine, dried over Na₂SO₄ and concentrated in vacuo. The residue was purified by silica gel chromatography (20% EtOAc/petroleum ether) to give 5-chloro-2-fluoro-4-(5-methyl-1,3,4-thiadiazol-2-yl)aniline I-8 as a white solid. LCMS (Method 3): m/z 244 (M+H)⁺ (ES⁺); at 1.06 min

### Intermediate 9 (I-9)

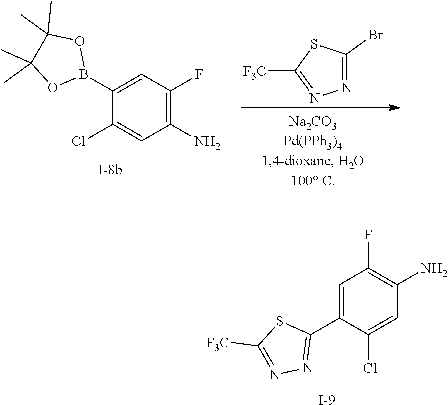

5-Chloro-2-fluoro-4-(5-(trifluoromethyl)-1,3,4-thiadiazol-2-yl)aniline I-9 was synthesised from 5-chloro-2-fluoro-4-(4,4,5,5-tetramethyl-1,3,2-dioxaborolan-2-yl) aniline I-8b and 2-bromo-5-(trifluoromethyl)-1,3,4-thiadiazole using a procedure essentially the same as I-8. ¹H NMR (400 MHz, DMSO-d6) δ 8.02 (d, J=12 Hz, 1H), 7.00 (d, J=8 Hz, 1H), 6.51 (s, 2H).

### Intermediate 10 (I-10)

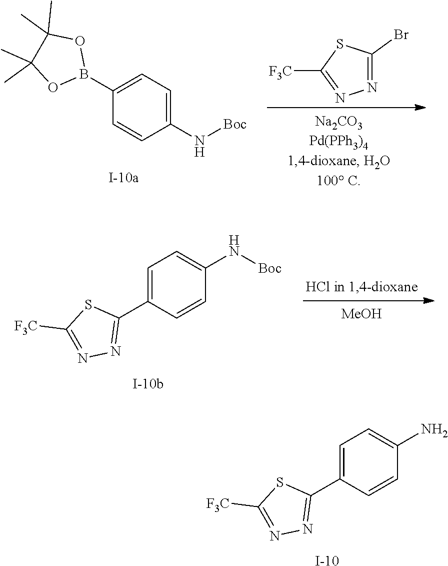

Step 1: A mixture of tert-butyl (4-(4,4,5,5-tetramethyl-1,3,2-dioxaborolan-2-yl)phenyl)carbamate I-10a (413.0 mg, 1.29 mmol), 2-bromo-5-(trifluoromethyl)-1,3,4-thiadiazole (200.0 mg, 0.86 mmol), K₂CO₃ (357.0 mg, 2.58 mmol) and Pd(PPh₃)₄ (100.0 mg, 0.08 mmol) in a mixture of 1,4-dioxane (5 ml) and water (1 ml) was heated at 100° C. for 5 h under N₂. The reaction mixture was diluted with water (10 ml) and extracted with DCM (3×10 ml). The combined organics were dried over Na₂SO₄ and concentrated in vacuo. The residue was purified by prep-TLC (33% EtOAc/petroleum ether) to give tert-butyl (4-(5-(trifluoromethyl)-1,3,4-thiadiazol-2-yl)phenyl)carbamate I-10b as a yellow solid. LCMS (Method 5) m/z 345.9 (M+H)⁺ (ES⁺), at 1.82 min.

Step 2: To a solution of tert-butyl (4-(5-(trifluoromethyl)-1,3,4-thiadiazol-2-yl)phenyl)carbamate I-10b (200.0 mg, 0.58 mmol) in MeOH (3 ml) was added a solution of HCl in 1,4-dioxane (725 ul, 4 M). The reaction was stirred at RT for 1 h. The pH was adjusted to 7-8 by addition of 2 M NaHCO₃. The mixture was diluted with water (5 ml) and extracted with DCM (2×10 ml). The combined organics were dried over Na₂SO₄ and concentrated in vacuo. The product was purified by prep-TLC (33% EtOAc/petroleum ether) to give 4-(5-(trifluoromethyl)-1,3,4-thiadiazol-2-yl)aniline I-10 as a yellow solid. LCMS (Method 5) m/z 245.9 (M+H)⁺ (ES⁺), at 1.17 min. ¹H NMR (400 MHz, DMSO-d6) δ 7.75-7.73 (m, 2H), 6.68-6.66 (m, 2H), 6.13 (s, 2H).

### Intermediate 11 (I-11)

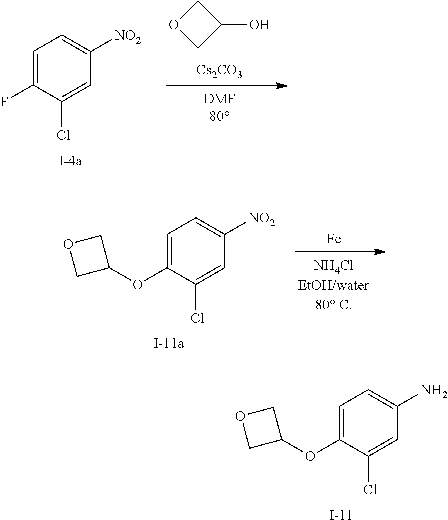

Step 1: To a solution of 2-chloro-1-fluoro-4-nitrobenzene I-4a (1.0 g, 5.7 mmol) and oxetan-3-ol (845 mg, 11.4 mmol) in DMF (20 ml) was added Cs₂CO₃ (5.6 g, 17.1 mol). The mixture was heated at 80° C. for 16 h. The reaction mixture was diluted with water and extracted with EtOAc (3×10 ml). The combined organics were washed with brine, dried over Na₂SO₄ and concentrated in vacuo. The residue was purified by silica gel column chromatography (10% EtOAc/petroleum ether) to give 3-(2-chloro-4-nitrophenoxy)oxetane I-11a as a yellow solid. ¹H NMR (400 MHz, DMSO-d6) δ 8.36 (t, J=2.2 Hz, 1H), 8.17 (ddd, J=9.1, 2.8, 1.2 Hz, 1H), 7.01 (d, J=9.1 Hz, 1H), 5.60-5.48 (m, 1H), 5.03-4.95 (m, 2H), 4.67-4.54 (m, 2H).

Step 2: To a solution of 3-(2-chloro-4-nitrophenoxy)oxetane I-11a (900 mg, 3.9 mmol) in a mixture of EtOH (10 ml) and saturated aqueous NH₄Cl (10 ml) was added iron powder (1.7 g, 31.4 mmol). The reaction was heated at 80° C. for 16 h, cooled and filtered. The product was extracted from the filtrate with EtOAc (2×20 ml). The combined organics were washed with brine, dried over Na₂SO₄ and concentrated in vacuo to give 3-chloro-4-(oxetan-3-yloxy)aniline I-11 as a grey solid. The product was used as is in the next step without any further purification. LCMS (Method 3) m/z 200.1 (M+H)⁺ (ES⁺), at 1.66 min. ¹H NMR (400 MHz, DMSO-d6) δ 6.65 (d, J=2.6 Hz, 1H), 6.54 (d, J=8.7 Hz, 1H), 6.43 (dd, J=8.7, 2.6 Hz, 1H), 5.10 (p, J=5.6 Hz, 1H), 4.95 (s, 2H), 4.84 (t, J=6.6 Hz, 2H), 4.54 (dd, J=7.2, 5.2 Hz, 2H).

### Intermediate 12 (I-12)

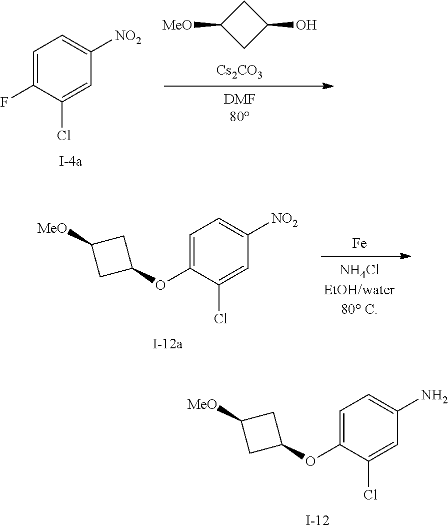

Step 1: 2-Chloro-1-((1s,3s)-3-methoxycyclobutoxy)-4-nitrobenzene I-12a was synthesised from 2-chloro-1-fluoro-4-nitrobenzene I-4a and (1s,3s)-3-methoxycyclobutan-1-ol using a procedure essentially the same as I-11a. ¹H NMR (400 MHz, DMSO-d6) δ 8.30 (d, J=2.8 Hz, 1H), 8.18 (dd, J=9.2, 2.8 Hz, 1H), 7.20 (d, J=9.2 Hz, 1H), 4.65 (p, J=6.9 Hz, 1H), 3.67 (p, J=6.8 Hz, 1H), 3.17 (s, 3H), 2.95 (ddp, J=9.8, 6.8, 3.8 Hz, 2H), 1.98-1.93 (m, 2H).

Step 2: 3-Chloro-4-((1s,3s)-3-methoxycyclobutoxy)aniline I-12 was synthesized from 2-chloro-1-((1s,3s)-3-methoxycyclobutoxy)-4-nitrobenzene I-12a using a procedure essentially the same as I-11. ¹H NMR (400 MHz, DMSO-d6) δ6.71 (d, J=8.8 Hz, 1H), 6.62 (d, J=2.8 Hz, 1H), 6.45 (dd, J=8.8, 2.8 Hz, 1H), 4.90 (s, 2H), 4.20 (p, J=7.0 Hz, 1H), 3.55 (p, J=6.8 Hz, 1H), 3.14 (s, 3H), 2.77-2.70 (m, 2H), 1.86-1.83 (m, 2H).

### Intermediate 13 (I-13)

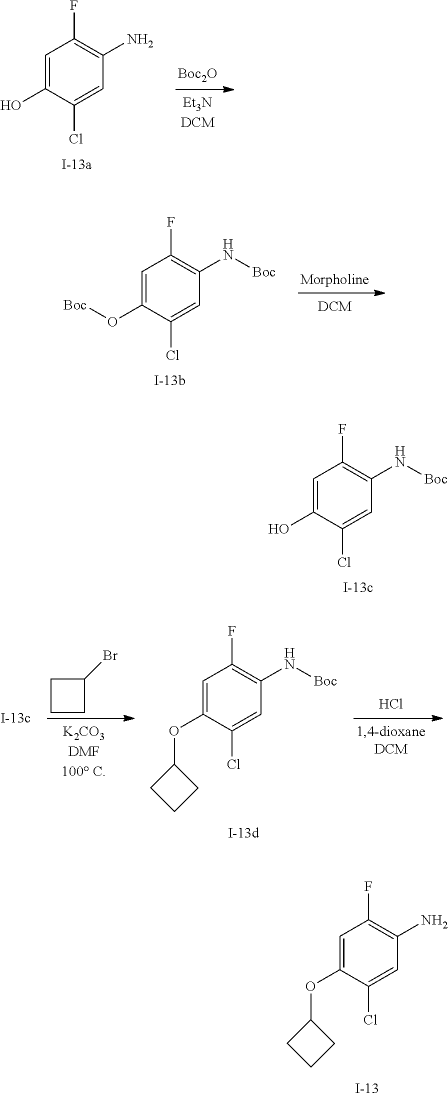

Step 1: To a solution of 4-amino-2-chloro-5-fluorophenol I-13a (0.250 g, 1.55 mmol) in DCM (5 ml) was added di-tert-butyl dicarbonate (371 mg, 1.70 mmol). The resultant mixture was stirred at RT for 48 h. THF (5 ml) was added and a further portion of di-tert-butyl dicarbonate (169 mg, 774 μmol) followed by Et₃N (216 μl, 1.55 mmol). The mixture was stirred at RT for 2 days. The mixture was concentrated in vacuo. The product was purified by chromatography on silica gel (0-100% EtOAc/isohexane) to afford tert-butyl (4-((tert-butoxycarbonyl)oxy)-5-chloro-2-fluorophenyl)carbamate I-13b. ¹H NMR (400 MHz, CDCl₃) δ 8.28 (d, J=8.1 Hz, 1H), 7.00 (d, J=10.9 Hz, 1H), 6.65 (s, 1H), 1.55 (s, 9H), 1.53 (s, 9H).

Step 2: To a solution of tert-butyl (4-((tert-butoxycarbonyl)oxy)-5-chloro-2-fluorophenyl)carbamate I-13b (200 mg, 547 μmol) in DCM (4 ml) was added morpholine (1.50 ml, 17.4 mmol) and the resulting mixture was stirred at RT overnight. The mixture was concentrated in vacuo. The product was purified by chromatography on silica gel (0-100% EtOAc/isohexane) to afford the product as a mixture containing morpholine. The material was taken up in DCM (15 ml) and washed with aq. 1 M HCl (2×15 ml). The organics were dried over MgSO₄ and concentrated in vacuo to afford tert-butyl (5-chloro-2-fluoro-4-hydroxyphenyl)carbamate I-13c as an orange oil. ¹H NMR (400 MHz, DMSO-d6) δ 10.43 (s, 1H), 8.76 (s, 1H), 7.43 (d, J=8.2 Hz, 1H), 6.78 (d, J=11.7 Hz, 1H), 1.43 (s, 9H).

Step 3: To a solution of tert-butyl (5-chloro-2-fluoro-4-hydroxyphenyl)carbamate I-13c (144 mg, 479 μmol) in DMF (1.0 ml) was added potassium carbonate (165 mg, 1.20 mmol) and bromocyclobutane (55.6 μl, 575 μmol) and the resulting mixture was stirred at 100° C. for 4 h. After cooling, the mixture was diluted with EtOAc (20 ml) and brine (20 ml). The layers were separated, and the aqueous layer was further extracted with EtOAc (20 ml). The combined organics were washed with brine (2×20 ml), dried over MgSO₄ and concentrated in vacuo. The product was purified by chromatography on silica gel (0-30% EtOAc/isohexane) to afford tert-butyl (5-chloro-4-cyclobutoxy-2-fluorophenyl)carbamate I-13d as a colourless solid. ¹H NMR (400 MHz, DMSO-d6) δ 8.89 (s, 1H), 7.56 (d, J=8.1 Hz, 1H), 6.92 (d, J=12.1 Hz, 1H), 4.74 (p, J=7.1 Hz, 1H), 2.48-2.40 (m, 2H), 2.11-1.97 (m, 2H), 1.85-1.72 (m, 1H), 1.69-1.56 (m, 1H), 1.44 (s, 9H).

Step 4: To a solution of tert-butyl (5-chloro-4-cyclobutoxy-2-fluorophenyl)carbamate I-13d (50 mg, 0.16 mmol) in DCM (1.0 ml) was added HCl in dioxane (1.00 ml, 4.0 molar, 4.00 mmol) and the resulting mixture was stirred at RT for 16 h. The mixture was loaded onto SCX (˜10 g) and the SCX was washed with MeOH (50 ml) and then the product was eluted with 0.7 M NH₃ in MeOH (50 ml) to afford 5-chloro-4-cyclobutoxy-2-fluoroaniline I-13 as a colourless oil. ¹H NMR (400 MHz, DMSO-d6) δ 6.82 (d, J=9.3 Hz, 1H), 6.77 (d, J=12.6 Hz, 1H), 4.89 (s, 2H), 4.62-4.52 (m, 1H), 2.42-2.29 (m, 2H), 2.08-1.92 (m, 2H), 1.82-1.69 (m, 1H), 1.64-1.50 (m, 1H)

### Intermediate 14 (I-14)

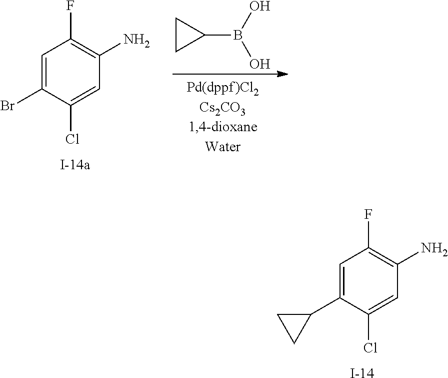

To a vial charged with 4-bromo-5-chloro-2-fluoroaniline I-14a (200 mg, 891 μmol), Cs₂CO₃ (871 mg, 2.67 mmol), cyclopropylboronic acid (76.5 mg, 891 μmol) and Pd(dppf)Cl₂ (65.2 mg, 89.1 μmol) was added 1,4-dioxane (5.0 ml) and water (0.5 ml). The vial was degassed for 5 min with N₂. The mixture was heated at 90° C. for 16 h. The mixture was cooled and diluted with EtOAc (30 ml), water (30 ml) and brine (10 ml). The layers were separated and the aqueous was extracted with EtOAc (2×20 ml). The combined organics were dried over MgSO₄ and concentrated in vacuo. The product was purified by chromatography on silica gel (0-100% DCM/isohexane) to afford 5-chloro-4-cyclopropyl-2-fluoroaniline I-14 as a clear yellow oil. ¹H NMR (400 MHz, DMSO-d6) δ 6.79 (d, J=8.5 Hz, 1H), 6.67 (d, J=12.6 Hz, 1H), 5.21 (s, 2H), 1.98-1.86 (m, 1H), 0.92-0.79 (m, 2H), 0.60-0.51 (m, 2H).

### Intermediate 15 (I-15)

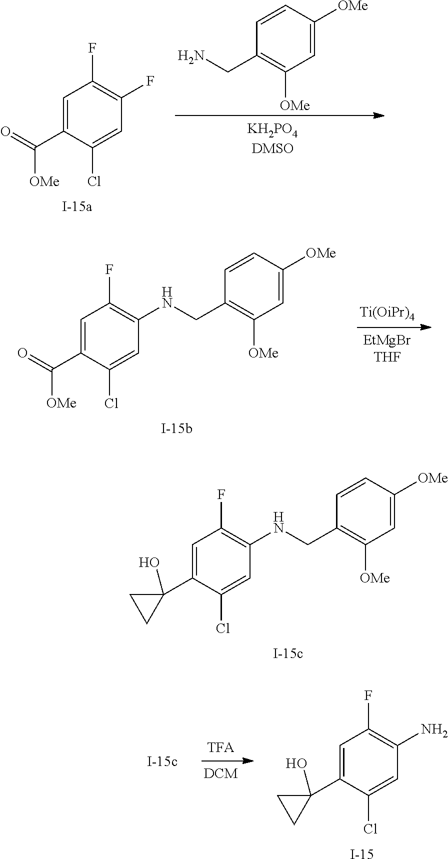

Step 1: To a solution of methyl 2-chloro-4,5-difluorobenzoate I-15a (377 mg, 1.83 mmol) in DMSO-d6 (3 ml) was added (2,4-dimethoxyphenyl)methanamine (333 μl, 2.19 mmol) and potassium dihydrogen phosphate (373 mg, 2.74 mmol). The reaction mixture was heated at 80° C. for 18 h. The mixture was cooled and diluted with a solution of 10% MeOH in DCM (30 ml) and water. The organics were washed with saturated NH₄Cl solution (3×50 ml). The solvent was concentrated in vacuo. The product was purified by chromatography on silica gel (0-10% MeOH/DCM) to afford methyl 2-chloro-4-((2,4-dimethoxybenzyl)amino)-5-fluorobenzoate I-15 b as a colourless solid. ¹H NMR (400 MHz, DMSO-d6) δ 7.54 (d, J=12.6 Hz, 1H), 7.08 (t, J=7.0 Hz, 2H), 6.62 (d, J=7.8 Hz, 1H), 6.58 (d, J=2.4 Hz, 1H), 6.48 (dd, J=8.4, 2.4 Hz, 1H), 4.27 (d, J=6.1 Hz, 2H), 3.84 (s, 3H), 3.74 (s, 3H), 3.73 (s, 3H).

Step 2: To a solution of methyl 2-chloro-4-((2,4-dimethoxybenzyl)amino)-5-fluorobenzoate I-15b (350 mg, 989 μmol) in THF (15 ml) was added titanium(IV) isopropoxide (420 μl, 1.39 mmol) followed by a dropwise addition of a solution of ethylmagnesium bromide in diethyl ether (923 μl, 3 M, 2.77 mmol). The reaction mixture was stirred for 2 h. A further portion of titanium(IV) isopropoxide (420 μl, 1.39 mmol) and ethylmagnesium bromide in diethyl ether (923 μl, 3 molar, 2.77 mmol) were added at RT and the reaction mixture was stirred for 16 h. The mixture was diluted with water (25 ml) and saturated NH₄Cl solution (20 ml). The product was extracted with EtOAc (3×100 ml). The combined organics were dried over Na₂SO₄ and concentrated in vacuo. The product was purified by sequential chromatography on silica gel (0-10% (0.7 M Ammonia/MeOH)/DCM) followed by (0-40% EtOAc/isohexane) to afford 1-(2-chloro-4-((2,4-dimethoxybenzyl)amino)-5-fluorophenyl)cyclopropan-1-ol I-15c as a colourless oil. ¹H NMR (500 MHz, DMSO-d6) δ 7.11-7.02 (m, 2H), 6.57 (d, J=2.4 Hz, 1H), 6.52-6.44 (m, 2H), 6.18 (dt, J=6.6, 3.4 Hz, 1H), 5.47 (s, 1H), 4.19 (d, J=6.2 Hz, 2H), 3.84 (s, 3H), 3.73 (s, 3H), 0.92-0.87 (m, 2H), 0.75-0.70 (m, 2H).

Step 3: To a solution of 1-(2-chloro-4-((2,4-dimethoxybenzyl)amino)-5-fluorophenyl)cyclopropan-1-ol I-15c (17 mg, 48 μmol) in DCM (3 ml) was added TFA (0.37 ml, 4.8 mmol). The reaction mixture was stirred at RT for 16 h. The mixture was concentrated in vacuo. The residue was dissolved in MeCN and the material was loaded onto a SCX cartridge. The cartridge was washed with MeOH and the product was eluted with 0.7 M NH₃ in MeOH solution to give 1-(4-amino-2-chloro-5-fluorophenyl)cyclopropan-1-ol I-5 as a colourless oil. ¹H NMR (400 MHz, DMSO-d6) δ 7.02 (d, J=11.9 Hz, 1H), 6.77 (d, J=8.3 Hz, 1H), 3.74 (s, 2H), 0.94-0.87 (m, 2H), 0.77-0.70 (m, 2H). 1 exchangeable proton not observed.

### Intermediate 17 (I-17)

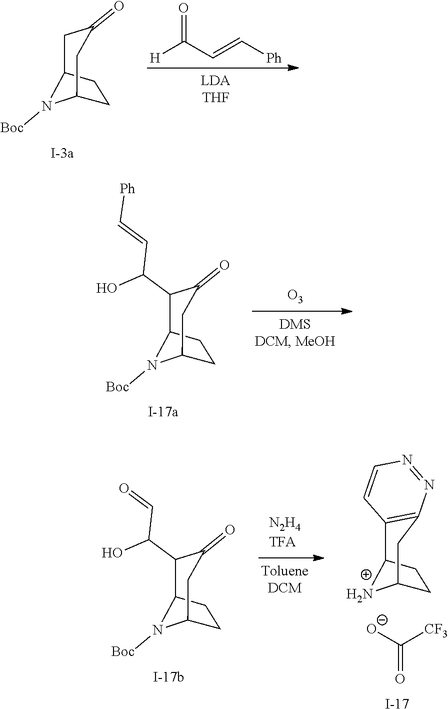

Step 1: To a solution of LDA (2.0 M in THF/heptane/ethylbenzene) (2.90 ml, 5.77 mmol) in THF (18 ml) at −78° C. was added a solution of tert-butyl-3-oxo-8-azabicyclo[3.2.1]octane-8-carboxylate I-3a (1.00 g, 4.44 mmol) in THF (2 ml) and the resulting mixture was left to stir at −78° C. for 30 min. A pre-cooled (−78° C.) solution of cinnamaldehyde (726 μL, 5.77 mmol) in THF (2 ml) was slowly added to the reaction mixture and stirring continued for 5 h, maintaining the temperature below −70° C. Saturated aqueous NaHCO₃ (50 ml) was added at −78° C. followed by MTBE (100 ml) and the layers were warmed to >0° C. and separated. The aqueous layer was extracted with MTBE (2×100 ml). The combined organics were dried over MgSO₄ and concentrated in vacuo to afford a yellow oil. The product was purified by chromatography on silica gel (0-100% EtOAc/isohexane) to afford tert-butyl (±)-2-((E)-1-hydroxy-3-phenylallyl)-3-oxo-8-azabicyclo[3.2.1]octane-8-carboxylate I-17a as a thick yellow oil. 1H NMR (400 MHz, DMSO-d6) δ 7.45-7.38 (m, 2H), 7.37-7.29 (m, 2H), 7.29-7.20 (m, 1H), 6.57 (d, J=15.9 Hz, 1H), 6.38 (dd, J=15.9, 6.4 Hz, 1H), 5.19 (d, J=5.1 Hz, 1H), 4.50 (br. S, 1H), 4.45 (d, J=6.5 Hz, 1H), 4.40-4.30 (m, 1H), 2.68-2.59 (m, 1H), 2.34 (d, J=8.6 Hz, 1H), 2.20 (dt, J=14.9, 1.8 Hz, 1H), 1.96-1.80 (m, 2H), 1.58-1.42 (m, 2H), 1.41 (s, 9H).

Step 2: A solution of tert-butyl (±)-2-((E)-1-hydroxy-3-phenylallyl)-3-oxo-8-azabicyclo[3.2.1]octane-8-carboxylate I-17a (630 mg, 1.50 mmol) in DCM (25 ml) and MeOH (25 ml) was stirred under N₂ and cooled to −78° C. Then oxygen was bubbled through the solution for 5 minutes, followed by ozone until the solution turned a deep blue colour, followed by oxygen until the solution became colourless. The reaction was then stirred under nitrogen and dimethyl sulfide (2.22 ml, 30.0 mmol) was added. The resulting mixture was allowed to stir overnight, slowly warming to rt. The solution was bubbled with nitrogen for 20 minutes, then the mixture was concentrated in vacuo. The product was purified by chromatography on silica gel (0-100% EtOAc/isohexane) to afford tert-butyl (±)-2-(1-hydroxy-2-oxoethyl)-3-oxo-8-azabicyclo[3.2.1]octane-8-carboxylate I-17b as a light yellow oil.

Step 3: To a solution of tert-butyl (±)-2-(1-hydroxy-2-oxoethyl)-3-oxo-8-azabicyclo[3.2.1]octane-8-carboxylate I-17b (289 mg, 918 μmol) in toluene (10 ml) was added hydrazine hydrate (90.6 μl, 1.01 mmol) and the resulting mixture was left to stir at 120° C. for 1 h. The solvent was removed in vacuo and the residue was taken up in MeOH (10 ml) and the solvent was removed. This was repeated 3 times. The residue was taken up in DCM (10 ml) and TFA (10 ml) was added and the resulting mixture was stirred at RT for 1 h. The solvent was removed in vacuo. The resulting residue was azeotroped with MeOH (3×10 ml) to afford (±)-6,7,8,9-tetrahydro-5H-5,8-epiminocyclohepta[c]pyridazine 2,2,2-trifluoroacetate as a brown oil. ¹H NMR (400 MHz, DMSO-d6) δ 9.62 (s, 1H), 9.46 (s, 1H), 9.14 (d, J=5.1 Hz, 1H), 7.61 (d, J=5.1 Hz, 1H), 5.01 (d, J=5.3 Hz, 1H), 4.52-4.47 (m, 1H), 3.58 (dd, J=18.2, 5.1 Hz, 1H), 3.24 (d, J=17.5 Hz, 1H), 2.32-2.19 (m, 2H), 2.11-1.97 (m, 1H), 1.96-1.84 (m, 1H).

### Intermediate 18 (I-18)

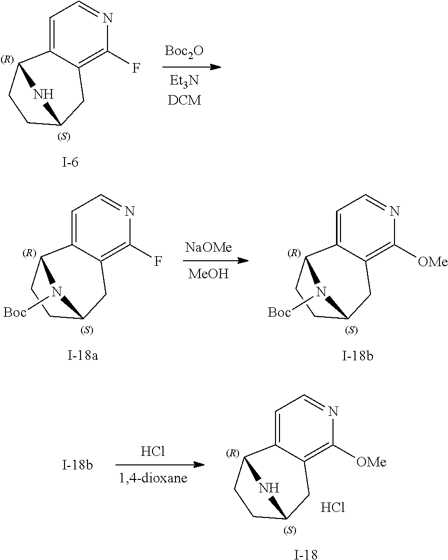

Step 1: To a solution of (5R,8S)-1-fluoro-6,7,8,9-tetrahydro-5H-5,8-epiminocyclohepta[c]pyridine I-6 (500 mg, 2.81 mmol) in DCM (5 ml) was added Et₃N (781 μl, 4.22 mmol) and di-tert-butyl dicarbonate (919 mg, 5.62 mol). The reaction was stirred at RT for 16 h then poured into water (30 ml) and extracted with EtOAc (3×10 ml). The combined organics were washed with brine, dried over Na₂SO₄ and concentrated in vacuo. The product was purified by chromatography on silica gel (10% EtOAc/petroleum ether) to afford tert-butyl(5R,8S)-1-fluoro-6,7,8,9-tetrahydro-5H-5,8-epiminocyclohepta[c]pyridine-10-carboxylate I-18a as a yellow oil. ¹H NMR (400 MHz, DMSO-d6) δ 7.99 (d, J=4.4 Hz, 1H), 7.20 (d, J=4.8 Hz, 1H), 4.92 (d, J=6.0 Hz, 1H), 4.45 (s, 1H), 3.07-3.02 (m, 1H), 2.56 (s, 1H), 2.17-2.09 (m, 2H), 1.81-1.76 (m, 1H), 1.70-1.65 (m, 1H), 1.32 (s, 9H).

Step 2: To a solution of tert-butyl (5R,8S)-1-fluoro-6,7,8,9-tetrahydro-5H-5,8-epiminocyclohepta[c]pyridine-10-carboxylate (200 mg, 0.72 mol) in MeOH (2 ml) was added NaOMe (117 mg, 2.16 mmol). The reaction was heated at 80° C. for 16 h, cooled and concentrated in vacuo. The product was purified by prep-TLC (20% EtOAc/petroleum ether) to give tert-butyl(5R,8S)-1-methoxy-6,7,8,9-tetrahydro-5H-5,8-epiminocyclohepta[c]pyridine-10-carboxylate I-18b as a yellow solid. ¹H NMR (400 MHz, DMSO-d6) δ 7.93 (d, J=5.2 Hz, 1H), 6.81 (d, J=5.2 Hz, 1H), 4.78 (d, J=5.6 Hz, 1H), 4.41 (s, 1H), 3.83 (s, 3H), 2.93 (d, J=16.4 Hz, 1H), 2.37 (d, J=17.4 Hz, 1H), 2.13-2.07 (m, 2H), 1.77-1.73 (m, 1H), 1.63-1.57 (m, 1H).

Step 3: To a solution of tert-butyl(5R,8S)-1-methoxy-6,7,8,9-tetrahydro-5H-5,8-epiminocyclohepta[c]pyridine-10-carboxylate I-18b (140 mg, 0.48 mmol) in 1,4-dioxane (1 ml) was added a solution of 4 M HCl in 1,4-dioxane (1 ml). The reaction was stirred at RT for 1 h. The reaction mixture was concentrated in vacuo to give (5R,8S)-1-methoxy-6,7,8,9-tetrahydro-5H-5,8-epiminocyclohepta[c]pyridine HCl salt as a red solid. ¹H NMR (400 MHz, DMSO-d6) δ 7.85 (d, J=5.2 Hz, 1H), 6.68 (d, J=5.2 Hz, 1H), 4.04 (d, J=5.2 Hz, 1H), 3.83-3.81 (m, 3H), 3.75 (t, J=6 Hz, 1H), 2.77-2.71 (m, 1H), 2.27-2.22 (m, 1H), 1.92-1.88 (m, 2H), 1.77-1.68 (1H), 1.48-1.41 (m, 1H). 1.32 (s, 1H).

### Intermediate 19 (I-19)

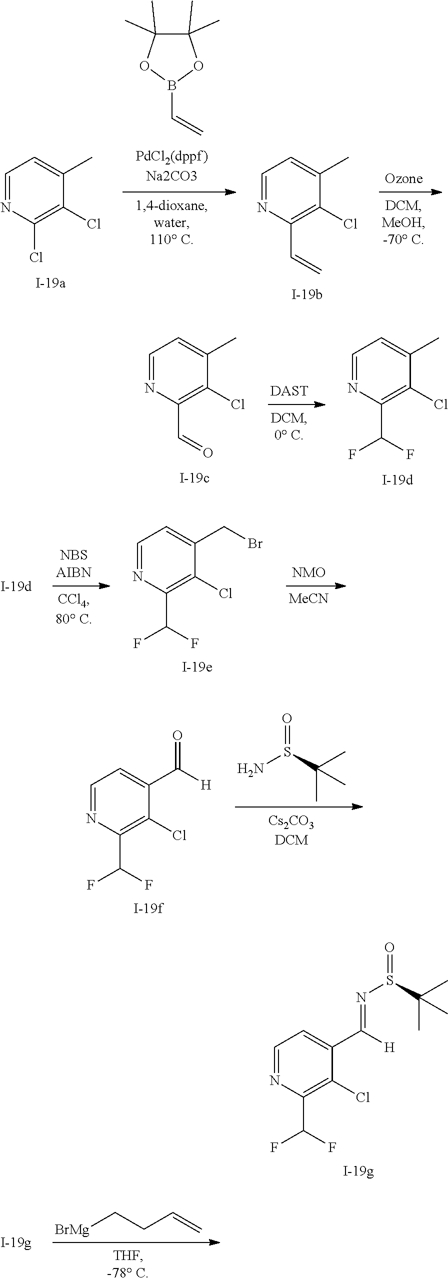

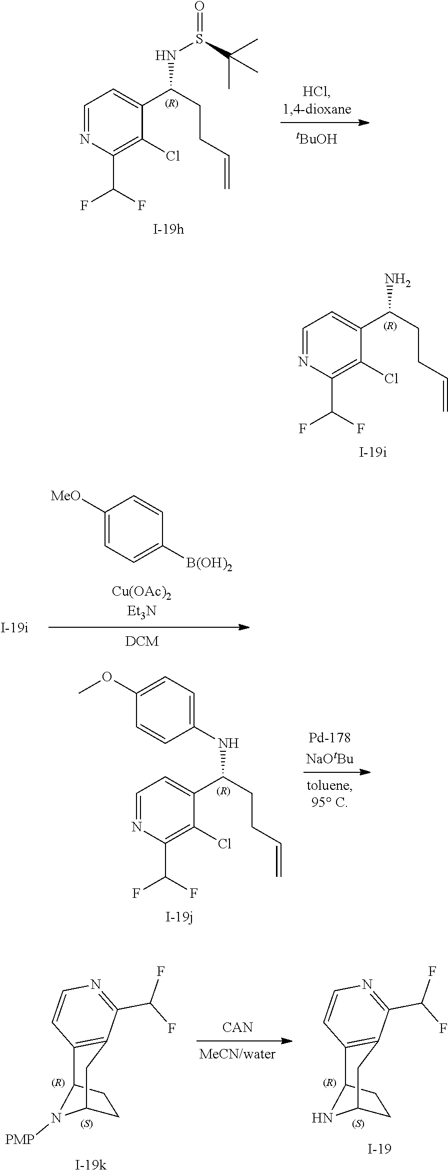

Step 1: To a mixture of 2,3-dichloro-4-methylpyridine I-19a (15.0 g, 92.59 mmol) and 4,4,5,5-tetramethyl-2-vinyl-1,3,2-dioxaborolane (14.3 g, 92.59 mmol) in a mixture of 1,4-dioxane (200 ml) and water (40 ml) were added Pd(dppf)Cl₂ (3.4 g, 6.43 umol) and Na₂CO₃ (29.4 g, 280 mmol). The mixture was heated at 110° C. under N₂ atmosphere for 16 h. The mixture was cooled and diluted with water (100 ml). The product was extracted with EtOAc (3×50 ml). The combined organics were washed with brine, dried over Na₂SO₄ and concentrated in vacuo. The product was purified by chromatography on silica gel (10% EtOAc/petroleum ether) to give 3-chloro-4-methyl-2-vinylpyridine I-19b as a colourless oil. ¹H NMR (400 MHz, DMSO-d6) δ 8.38 (d, J=4.8 Hz, 1H), 7.32 (d, J=4.8 Hz, 1H), 7.24 (dd, J=18.8, 25.2 Hz, 1H), 6.40 (dd, J=3.2, 25.6 Hz, 1H), 5.60 (dd, J=2.4 Hz, 10.8 Hz, 1H), 2.37 (s, 3H).

Step 2: A solution of 3-chloro-4-methyl-2-vinylpyridine I-19b (11 g, 71.61 mol) in mixture of DCM (1 ml) and MeOH (25 ml) at −78° C. was bubbled with O₃ for 30 min. The reaction was quenched with dimethyl sulfide and the mixture was concentrated in vacuo. The residue was purified by chromatography on silica gel (10% EtOAc/petroleum ether) to give 3-chloro-4-methylpicolinaldehyde I-19c as a colorless oil. ¹H NMR (400 MHz, DMSO-d6) δ 10.15 (s, 1H), 8.62 (d, J=4.8 Hz, 1H), 7.69 (d, J=5.2 Hz, 1H), 2.45 (s, 3H).

Step 3: To a solution of 3-chloro-4-methylpicolinaldehyde I-19c (5 g, 32.14 mmol) in DCM (30 ml) at 0° C. was added DAST (15.5 g, 96.41 mmol). The mixture was stirred at 0° C. for 2 h then the reaction mixture was poured onto saturated aqueous NaHCO₃ solution (30 ml). The aqueous was extracted with DCM (3×30 ml) and the combined organics were dried with Na₂SO₄ and concentrated in vacuo. The product was purified by chromatography on silica gel (10% EtOAc/petroleum Ether) to give 3-chloro-2-(difluoromethyl)-4-methylpyridine 1-19d as a light yellow oil. ¹H NMR (400 MHz, DMSO-d6) δ 8.50 (d, J=4.8 Hz, 1H), 7.62 (d, J=4.4 Hz, 1H), 7.18 (t, J=61.2 Hz, 1H), 2.43 (s, 3H).

Step 4: To a mixture of 3-chloro-2-(difluoromethyl)-4-methylpyridine I-19d (1.9 g, 10.70 mmol) and AlBN (265 mg, 1.07 mmol) in CCl₄ (20 ml) was added NBS (2.3 g, 12.84 mmol). The reaction was heated at 80° C. for 16 h. The reaction mixture was concentrated in vacuo. The product was purified by chromatography on silica gel (5% EtOAc/petroleum Ether) to give 4-(bromomethyl)-3-chloro-2-(difluoromethyl)pyridine I-19e as a colorless oil. ¹H NMR: (400 MHz, DMSO-d6) δ 8.65 (d, J=4.8 Hz, 1H), 7.85 (d, J=4.4 Hz, 1H), 7.24 (t, J=53.2 Hz, 1H), 4.78 (s, 2H).

Step 5: To a solution of 4-(bromomethyl)-3-chloro-2-(difluoromethyl)pyridine I-19e (1 g, 3.90 mmol) in MeCN (10 ml) was added NMO (913 mg, 7.80 mmol). After stirring at room temperature for 16 h, the reaction mixture was concentrated in vacuo. The residue was purified by silica gel column (10% EtOAc/petroleum Ether) to give 3-chloro-2-(difluoromethyl)isonicotinaldehyde I-19f as a white solid. ¹H NMR (400 MHz, DMSO-d6) δ 10.36 (s, 1H), 8.85 (d, J=4.8 Hz, 1H), 7.93 (d, J=4.8 Hz, 1H), 7.34 (t, J=61.2 Hz, 1H).

Step 6: (S)—N-((3-Chloro-2-(difluoromethyl)pyridine-4-yl)methylene)-2-methylpropane-2-sulfinamide I-19g was synthesised from 3-chloro-2-(difluoromethyl)isonicotinaldehyde I-19f using a procedure essentially the same as for I-7j. ¹H NMR (400 MHz, DMSO-d6) δ 8.87 (s, 1H), 8.79 (d, J=5.2 Hz, 1H), 8.11 (d, J=4.8 Hz, 1H), 7.31 (t, J=53.2 Hz, 1H), 1.22 (s, 9H) Step 7: To a solution of (S)—N-((3-chloro-2-(difluoromethyl)pyridine-4-yl)methylene)-2-methylpropane-2-sulfinamide I-19g (600 mg, 2.04 mmol) in THF (8 ml) at −78° C. was added a solution of but-3-en-1-ylmagnesium bromide (8.1 ml, 0.5 M, 4.07 mmol) in THF (8 ml) dropwise. The mixture was stirred at −78° C. for 2 h then the reaction was quenched with saturated aqueous NH₄Cl solution (10 ml). The product was extracted with EtOAc (3×10 ml). The combined organics were washed with brine, dried over Na₂SO₄ and concentrated in vacuo. The product was purified by chromatography on silica gel (2% MeOH/DCM) to give (S)—N—((R)-1-(3-chloro-2-(difluoromethyl)pyridine-4-yl)pent-4-en-1-yl)-2-methylpropane-2-sulfinamide I-19 h as a light yellow solid. ¹H NMR (400 MHz, DMSO-d6) δ 8.65 (d, J=4.8 Hz, 1H), 7.75 (d, J=5.2 Hz, 1H), 7.21 (t, J=53.2 Hz, 1H), 5.89 (d, J=7.2 Hz, 1H), 5.85-5.77 (m, 1H), 5.12-5.08 (m, 1H), 5.03-4.99 (m, 1H), 4.74-4.68 (m, 1H), 2.27-2.09 (m, 2H), 1.95-1.86 (m, 1H), 1.80-1.71 (m, 1H), 1.07 (s, 9H).

Step 8: (R)-1-(3-Chloro-2-(difluoromethyl)pyridin-4-yl)pent-4-en-1-amine I-19i was synthesised from (S)—N—((R)-1-(3-chloro-2-(difluoromethyl)pyridine-4-yl)pent-4-en-1-yl)-2-methylpropane-2-sulfinamide I-19 h using a procedure that is essentially the same as for I-7m. ¹H NMR (400 MHz, DMSO-d6) δ 8.60 (d, J=4.8 Hz, 1H), 7.85 (d, J=5.2 Hz, 1H), 7.20 (t, J=53.2 Hz, 1H), 5.86-5.76 (m, 1H), 5.05-4.94 (m, 2H), 4.26-4.22 (m, 1H), 2.20-2.04 (m, 4H), 1.69-1.60 (m, 1H), 1.58-1.51 (m, 1H).

Step 9: (R)—N-(1-(3-Chloro-2-(difluoromethyl)pyridin-4-yl)pent-4-en-1-yl)-4-methoxyaniline I-19j was synthesised from I-1-(3-chloro-2-(difluoromethyl)pyridine-4-yl)pent-4-en-1-amine I-19i using a procedure essentially the same as for I-7m. ¹H NMR (400 MHz, DMSO-d6) δ 8.54 (d, J=5.2 Hz, 1H), 7.63 (d, J=4.8 Hz, 1H), 7.22 (t, J=53.6 Hz, 1H), 6.64 (d, J=8.8 Hz, 2H), 6.38 (d, J=9.2 Hz, 2H), 6.10 (d, J=8.4 Hz, 1H), 5.90-5.80 (m, 1H), 5.05-4.98 (m, 2H), 4.75-4.70 (m, 1H), 3.57 (s, 3H), 2.35-2.15 (m, 2H), 1.83-1.70 (m, 2H)

Step 10: A mixture of (R)—N-(1-(3-chloro-2-(difluoromethyl)pyridin-4-yl)pent-4-en-1-yl)-4-methoxyaniline I-19j (150 mg, 0.43 mmol), NaO^{t}Bu (61 mg, 0.64 mmol) and Pd-178 (10 mg, 0.02 mmol) in toluene (5 ml) was heated at 95° C. for 3 h. The mixture was allowed to cool to RT and concentrated in vacuo. The residue was purified by prep-TLC (50% EtOAc/petroleum ether) to give (5R,8S)-1-(difluoromethyl)-10-(4-methoxyphenyl)-6,7,8,9-tetrahydro-5H-5,8-epiminocyclohepta[c]pyridine I-19k as an orange solid. ¹H NMR (400 MHz, DMSO-d6) δ 8.37 (d, J=4.8 Hz, 1H), 7.47 (d, J=4.8 Hz, 1H), 6.98-6.80 (m, 3H), 6.72-6.70 (m, 2H), 4.94 (d, J=5.6 Hz, 1H), 4.56 (t, J=5.2 Hz, 1H), 3.61 (s, 3H), 3.20 (dd, J=4.0, 18.0 Hz, 1H), 2.62 (d, J=17.6 Hz, 1H), 2.33-2.24 (m, 2H), 1.88-1.83 (m, 1H), 1.81-1.72 (m, 1H).

Step 11: (5R,8S)-1-(difluoromethyl)-6,7,8,9-tetrahydro-5H-5,8-epiminocyclohepta[c]pyridine 1-19 was synthesised from (5R,8S)-1-(difluoromethyl)-10-(4-methoxyphenyl)-6,7,8,9-tetrahydro-5H-5,8-epiminocyclohepta[c]pyridine I-19k using a procedure essentially the same as I-6. ¹H NMR (400 MHz, DMSO-d6) δ 8.33 (d, J=4.8 Hz, 1H), 7.22 (d, J=4.8 Hz, 1H), 6.89 (t, J=54.0 Hz, 1H), 4.18 (d, J=5.6 Hz, 1H), 3.79 (t, J=5.2 Hz, 1H), 3.11 (dd, J=4.4 Hz, 17.6 Hz, 1H), 2.74 (s, 1H), 2.67 (d, J=17.2 Hz, 1H), 1.99-1.89 (m, 2H), 1.75-1.70 (m, 1H), 1.51-1.45 (m, 1H).

### Intermediate 20 (I-20)

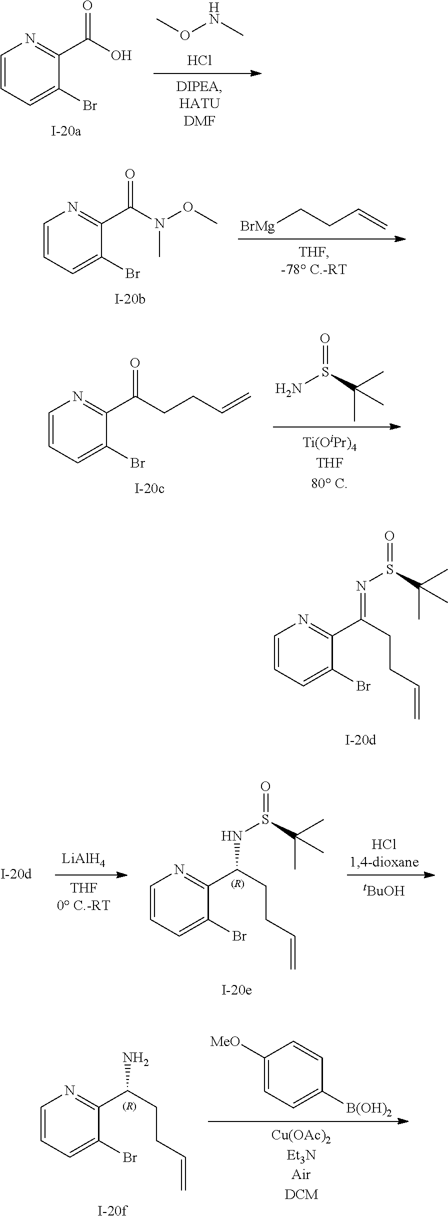

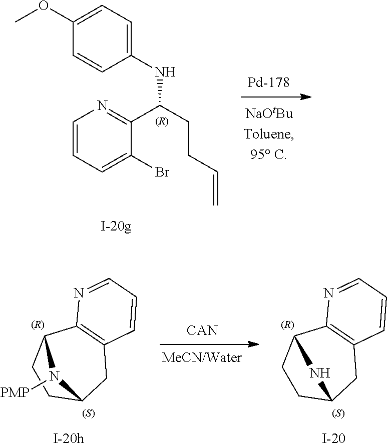

Step 1: To a solution of 3-bromopicolinic acid I-20a (5.0 g, 24.8 mmol) in DMF (60 ml) was added N,O-dimethylhydroxylamine hydrochloride (3.6 g, 37.2 mmol), DIPEA (21.6 ml, 124 mmol) and HATU (14.1 g, 37.2 mmol). The reaction was stirred at RT for 16 h then poured into water and the product was extracted with EtOAc (2×100 ml). The combined organics were washed with brine, dried over Na₂SO₄ and concentrated in vacuo. The product was purified by chromatography on silica gel (25-50% EtOAc/petroleum ether) to give 3-bromo-N-methoxy-N-methylpicolinamide I-20b as a colorless oil. ¹H NMR (400 MHz, DMSO-d6) δ 8.59-8.57 (m, 1H), 8.18-8.15 (m, 1H), 7.45-7.42 (m, 1H), 3.49 (s, 3H), 3.30 (s, 3H).

Step 2: To a solution of 3-bromo-N-methoxy-N-methylpicolinamide I-20b (5.8 g, 23.8 mmol) in THF (50 ml) at −78° C. was added a solution of but-3-en-1-ylmagnesium bromide (52 ml 0.5 M in THF, 26.1 mmol) in THF (52 ml) dropwise. The reaction mixture was allowed to warm slowly to RT and stirred for 16 h. The reaction mixture was poured into saturated aqueous NH₄Cl (100 ml) and extracted with EtOAc (2×200 ml). The combined organics were washed with brine, dried over Na₂SO₄ and concentrated in vacuo. The product was purified by chromatography on silica gel (5% EtOAc/petroleum ether) to give 1-(3-bromopyridin-2-yl)pent-4-en-1-one I-20c as a yellow oil. ¹H NMR (400 MHz, DMSO-d6) δ 8.66-8.64 (m, 1H), 8.24-8.21 (m, 1H), 7.54-7.50 (m, 1H), 5.91-5.81 (m, 1H), 5.09-4.96 (m, 2H), 3.14 (t, J=7.2 Hz, 2H), 2.39-2.33 (m, 2H).

Step 3: To a solution of 1-(3-bromopyridin-2-yl)pent-4-en-1-one I-20c (3.4 g, 14.1 mmol) in THF (50 ml) was added (S)-2-methylpropane-2-sulfinamide (5.1 g, 42.4 mmol) and titanium tetraisopropoxide (20.1 g, 70.8 mmol). The reaction mixture was heated at 80° C. for 16 h then poured into water and extracted with EtOAc (2×100 ml). The combined organics were washed with brine, dried over Na₂SO₄ and concentrated in vacuo. The product was purified by chromatography on silica gel (10-20% EtOAc/petroleum ether) to give (S)—N-(1-(3-bromopyridin-2-yl)pent-4-en-1-ylidene)-2-methylpropane-2-sulfinamide I-20d as a yellow oil. ¹H NMR (400 MHz, DMSO-d6) δ 8.53 (d, J=4.8 Hz, 1H), 8.06 (d, J=8.4 Hz, 1H), 7.36-7.33 (m, 1H), 5.93-5.84 (m, 1H), 5.13-4.98 (m, 2H), 2.89-2.67 (m, 2H), 2.43-2.38 (m, 2H), 1.16 (s, 9H).

Step 4: To a solution of (S)—N-(1-(3-bromopyridin-2-yl)pent-4-en-1-ylidene)-2-methylpropane-2-sulfinamide I-20d (2 g, 5.9 mmol) in THF (20 ml) at 0° C. was added LiAlH₄ (333 mg, 8.8 mmol). The reaction was allowed to warm slowly to RT and stirred for 2 h. After completion, the reaction was quenched with careful addition of water (20 ml) and the product was extracted with EtOAc (2×30 ml). The combined organics were washed with brine, dried over Na₂SO₄ and concentrated in vacuo. The product was purified by chromatography on silica gel (20-33% EtOAc/petroleum ether) to give (S)—N—((R)-1-(3-bromopyridin-2-yl)pent-4-en-1-yl)-2-methylpropane-2-sulfinamide I-20e as a yellow oil. ¹HNMR (400 MHz, DMSO-d6) δ 8.59-8.57 (m, 1H), 8.05-8.03 (m, 1H), 7.25 (dd, J=8.0, 4.4 Hz, 1H), 5.84-5.74 (m, 1H), 5.48 (d, J=8.4 Hz, 1H), 5.04-4.95 (m, 2H), 4.82-4.77 (m, 1H), 2.10-2.00 (m, 2H), 1.99-1.94 (m, 2H), 1.01 (s, 9H).

Step 5: (R)-1-(3-Bromopyridin-2-yl)pent-4-en-1-amine I-20f was synthesised from (S)—N—((R)-1-(3-bromopyridin-2-yl)pent-4-en-1-yl)-2-methylpropane-2-sulfinamide I-20e using a procedure that is essentially the same as I-71. LCMS (Method 5) m/z 241.1 (M+H)⁺ (ES⁺), at 0.64 min.

Step 6: To a solution of (R)-1-(3-bromopyridin-2-yl)pent-4-en-1-amine I-20f (430 mg, 1.7 mmol) in DCM (15 ml) was added (4-methoxyphenyl)boronic acid (1.1 g, 7.5 mmol), Et₃N (1.51 ml, 11.04 mmol) and Cu(OAc)₂ (648 mg, 3.56 mmol). The reaction was stirred at RT for 2 days under O₂ atmosphere. The reaction solution was poured into aqueous NaOH (10 ml, 2 M) and filtered through celite. The aqueous layer was extracted with DCM (2×30 ml), the combined organics were washed with brine, dried over Na₂SO₄ and concentrated in vacuo. The product was purified by prep-TLC (33% EtOAc/petroleum ether) to give (R)—N-(1-(3-bromopyridin-2-yl)pent-4-en-1-yl)-4-methoxyaniline I-20g as a yellow oil. ¹H NMR (400 MHz, DMSO-d6) δ 8.52-8.50 (m, 1H), 8.01-7.99 (m, 1H), 7.19 (dd, J=7.2, 4.8 Hz, 1H), 6.64-6.61 (m, 2H), 6.54-6.50 (m, 2H), 5.88-5.78 (m, 1H), 5.56 (d, J=10.0 Hz, 1H), 5.03-4.95 (m, 2H), 4.88-4.82 (m, 1H), 3.58 (s, 3H), 2.26-2.18 (m, 1H), 2.10-2.03 (m, 1H), 1.85-1.78 (m, 2H).

Step 7: To a solution of (R)—N-(1-(3-bromopyridin-2-yl)pent-4-en-1-yl)-4-methoxyaniline I-20g (180 mg, 0.52 mmol) in toluene (4 ml) was added NaO^{t}Bu (75 mg, 0.78 mmol) and Pd-178 (12.3 mg, 0.03 mmol). The reaction solution was heated at 95° C. for 2 h under a N₂ atmosphere. After completion the reaction mixture was poured into water and extracted with EtOAc (2×20 ml). The combined organics were washed with brine, dried over Na₂SO₄ and concentrated in vacuo. The product was purified by prep-TLC (50% EtOAc/petroleum ether) to give (6S,9R)-10-(4-methoxyphenyl)-6,7,8,9-tetrahydro-5H-6,9-epiminocyclohepta[b]pyridine 120 h as a pink solid. ¹H NMR (400 MHz, DMSO-d6) δ 8.26-8.24 (m, 1H), 7.34-7.32 (m, 1H), 7.10-7.06 (m, 1H), 6.75-6.68 (m, 4H), 4.68 (d, J=5.6 Hz, 1H), 4.49 (t, J=5.6 Hz, 1H), 3.60 (s, 3H), 3.18-3.10 (m, 1H), 2.43 (d, J=17.2 Hz, 1H), 2.30-2.26 (m, 2H), 1.84 (t, J=9.2 Hz, 1H), 1.73-1.66 (m, 1H).

Step 8: (6S,9R)-6,7,8,9-Tetrahydro-5H-6,9-epiminocyclohepta[b]pyridine I-20 was synthesised from (6S,9R)-10-(4-methoxyphenyl)-6,7,8,9-tetrahydro-5H-6,9-epiminocyclohepta[b]pyridine 120 h using a procedure essentially the same as I-6. ¹H NMR (400 MHz, DMSO-d6) δ 8.19 (d, J=4.8 Hz, 1H), 7.41 (d, J=7.6 Hz, 1H), 7.11-7.08 (m, 1H), 4.09 (br. s, 1H), 3.72 (br. s, 1H), 3.05-3.00 (m, 2H), 1.96-1.91 (m, 2H), 1.73 (t, J=9.6 Hz, 1H), 1.46-1.41 (m, 1H)

### Intermediate 21 (I-21)

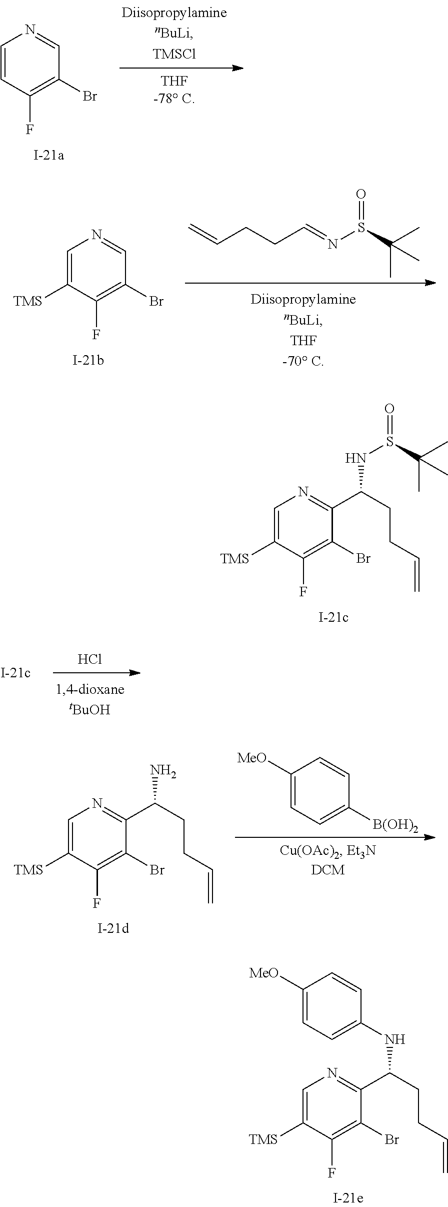

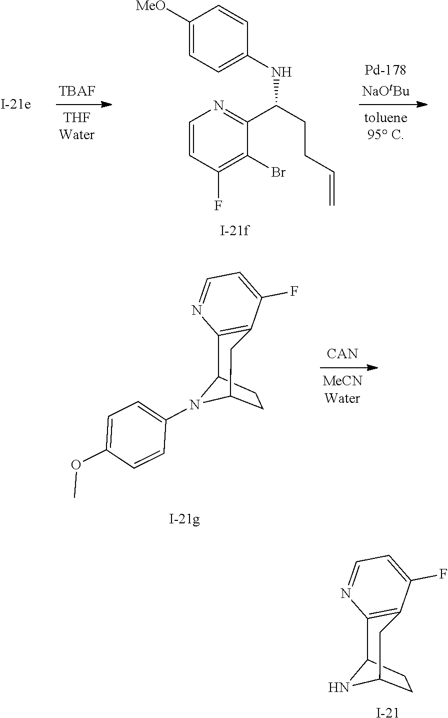

Step 1: To a solution of diisopropylamine (317 mg, 3.14 mmol) in THF (8 ml) at −78° C. under N₂ atmosphere was added a solution of ⁿBuLi (1.26 mL, 2.5 M in hexanes, 3.14 mmol). The mixture was stirred at −78° C. for 30 min then a solution of 3-bromo-4-fluoropyridine I-21a (500 mg, 2.86 mmol) in THF (8 ml) was added. The reaction was stirred at −78° C. for 30 min then TMS-Cl (376 mg, 3.43 mmol) was added and the reaction was stirred at −78° C. for a further 30 min. After completion, the reaction was poured into a cooled solution of 1 M HCl (20 ml) at 0° C. The product was extracted with EtOAc (2×20 ml). The combined organics were concentrated in vacuo and the residue purified by prep-TLC (20% EtOAc/petroleum ether) to give 3-bromo-4-fluoro-5-(trimethylsilyl)pyridine I-21b as a white solid. ¹H NMR (400 MHz, DMSO-d6) δ 8.82 (d, J=10.0 Hz, 1H), 8.50 (d, J=8.0 Hz, 1H), 0.35 (s, 9H).

Step 2: To a solution of diisopropylamine (3.12 ml, 22.2 mmol) in THF (10 ml) at −78° C. under N₂ atmosphere was added ⁿBuLi (8.9 mL, 2.5 M in hexanes, 22.2 mmol). The mixture was stirred at −78° C. for 30 min then a solution of 3-bromo-4-fluoro-5-(trimethylsilyl)pyridine I-21b (3.65 g, 14.8 mmol) in THF (40 ml) was added. The reaction was stirred at −78° C. for 2 h then a solution of (S)-2-methyl-N-(pent-4-en-1-ylidene)propane-2-sulfinamide (3.6 g, 17.7 mmol) in THF (20 ml) was added. The synthesis of (S)-2-methyl-N-(pent-4-en-1-ylidene)propane-2-sulfinamide is described in J. Am. Chem. Soc. 2020, 142, 21, 9850-9857. The reaction was stirred at −78° C. for 2 h then poured into a cooled solution of 1 M HCl (50 ml) at 0° C. The product was extracted with EtOAc (2×50 ml) and the combined organics were concentrated in vacuo. The product was purified by chromatography on silica gel (20% EtOAc/petroleum ether) to give (S)—N—((R)-1-(3-bromo-4-fluoro-5-(trimethylsilyl)pyridin-2-yl)pent-4-en-1-yl)-2-methylpropane-2-sulfinamide I-21c as a yellow solid. ¹H NMR (400 MHz, DMSO-d6) δ 8.24 (d, J=8.0 Hz, 1H), 5.84-5.74 (m, 1H), 5.06-4.96 (m, 2H), 4.80-4.76 (br. s, 1H), 2.20-2.09 (m, 2H), 2.05-1.86 (m, 2H), 1.00 (s, 9H), 0.31 (s, 9H)

Step 3: (R)-1-(3-Bromo-4-fluoro-5-(trimethylsilyl)pyridine-2-yl)pent-4-en-1-amine I-21d was synthesised from (S)—N—((R)-1-(3-bromo-4-fluoro-5-(trimethylsilyl)pyridin-2-yl)pent-4-en-1-yl)-2-methylpropane-2-sulfinamide I-21c using a procedure essentially the same as I-71. LCMS (Method 5) m/z 331.1 (M+H)⁺ (ES⁺), at 2.89 min

Step 4: (R)—N-(1-(3-Bromo-4-fluoro-5-(trimethylsilyl)pyridin-2-yl)pent-4-en-1-yl)-4-methoxyaniline I-21e was synthesised from (R)-1-(3-bromo-4-fluoro-5-(trimethylsilyl)pyridine-2-yl)pent-4-en-1-amine I-21d using a procedure essentially the same as I-7m. LCMS (Method 5) m/z 437.2 (M+H)⁺ (ES⁺), at 3.26 min

Step 5: To a solution of (R)—N-(1-(3-bromo-4-fluoro-5-(trimethylsilyl)pyridin-2-yl)pent-4-en-1-yl)-4-methoxyaniline I-21e (340 mg, 0.78 mmol, 1 eq) in THF (2 ml) and water (2 ml) at 0° C. was added a solution of TBAF (850 ul, 1 M in THF, 0.85 mmol). The reaction was heated at 60° C. for 2 h. The reaction was cooled and the concentrated in vacuo. The residue was partitioned between EtOAc and water and the product was extracted with EtOAc (2×20 ml). The combined organics were washed with brine, dried over Na₂SO₄ and concentrated in vacuo. The product was purified by chromatography on silica gel (10% EtOAc/petroleum ether) to give (R)—N-(1-(3-bromo-4-fluoropyridin-2-yl)pent-4-en-1-yl)-4-methoxyaniline I-21f as a yellow oil. LCMS (Method 5) m/z 365.0 (M+H)⁺ (ES⁺), at 3.75 min

Step 6: (6S,9R)-4-Fluoro-10-(4-methoxyphenyl)-6,7,8,9-tetrahydro-5H-6,9-epiminocyclohepta[b]pyridine I-21g was synthesised from (R)—N-(1-(3-bromo-4-fluoropyridin-2-yl)pent-4-en-1-yl)-4-methoxyaniline I-21f using a procedure essentially the same as for I-20 h. LCMS (Method 5) m/z 285.2 (M+H)⁺ (ES⁺), at 2.43 min

Step 7: (6S,9R)-4-Fluoro-6,7,8,9-tetrahydro-5H-6,9-epiminocyclohepta[b]pyridine I-21 was synthesised from (6S,9R)-4-fluoro-10-(4-methoxyphenyl)-6,7,8,9-tetrahydro-5H-6,9-epiminocyclohepta[b]pyridine I-21g using a procedure essentially the same as I-6. LCMS (Method 5) m/z 179.1 (M+H)⁺ (ES⁺), at 2.23 min Intermediate 22 (1-22)

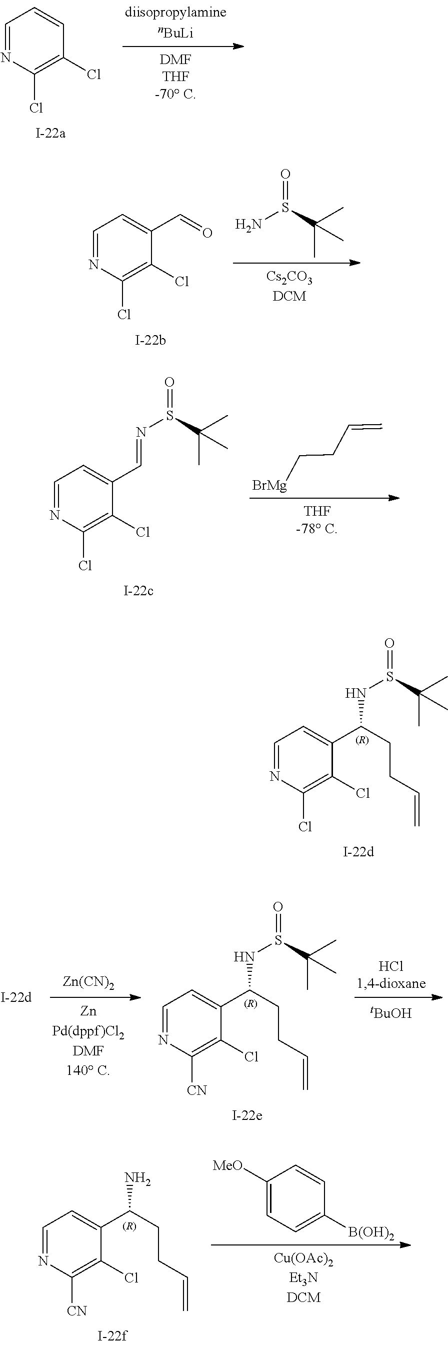

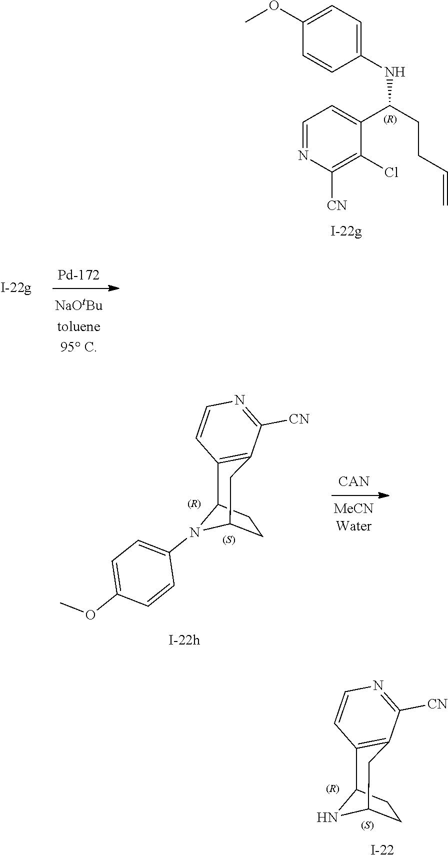

Step 1: To a solution of diisopropylamine (30.7 g, 304 mmol) in dry THF (300 ml) at −70° C. was added ⁿBuLi (121 ml, 2.5 M in hexanes, 303 mmol). After stirring at −70° C. for 30 min, a solution of 2,3-dichloropyridine I-22a (30 g, 203 mmol) in dry THF (100 ml) was added. The reaction was stirred for a further 1 h at −70° C. then dry DMF (39.2 ml, 304 mmol) was added and the mixture was stirred for a further 1 h. The reaction was allowed to warm to RT and poured into saturated aqueous NH₄Cl (200 ml). The product was extracted with EtOAc (3×200 ml) and the combined organics were washed with water and brine (200 ml), dried over Na₂SO₄ and concentrated in vacuo. The product was purified by chromatography on silica gel (20% EtOAc/petroleum Ether) to give 2,3-dichloroisonicotinaldehyde I-22b as a yellow solid. ¹H NMR (400 MHz, DMSO-d6) δ 10.28 (s, 1H), 8.58 (d, J=4.8 Hz, 1H), 7.75 (d, J=4.8 Hz, 1H).

Step 2: (S)—N-((2,3-Dichloropyridin-4-yl)methylene)-2-methylpropane-2-sulfinamide I-22c was synthesised from 2,3-dichloroisonicotinaldehyde I-22b using a procedure essentially the same as for I-7j. ¹H NMR (400 MHz, DMSO-d6) δ 8.80 (s, 1H), 8.52 (d, J=4.8 Hz, 1H), 7.93 (d, J=4.8 Hz, 1H), 1.21 (s, 9H) Step 3: To a solution of (S)—N-((2,3-dichloropyridin-4-yl)methylene)-2-methylpropane-2-sulfinamide I-22c (34.2 g, 123 mmol) in THF (400 ml) at −70° C. was added a solution of but-3-en-1-ylmagnesium bromide (68 ml, 2.0 M in THF, 136 mmol). The reaction was stirred at −70° C. for 4 h then quenched by addition of saturated aqueous NH₄Cl solution (200 ml). The product was extracted with EtOAc (3×200 ml) and the combined organics were washed with water and brine (200 ml), dried over Na₂SO₄, concentrated in vacuo. The product was purified by chromatography on silica gel (20% EtOAc/petroleum Ether) to give (S)—N—((R)-1-(2,3-dichloropyridin-4-yl)pent-4-en-1-yl)-2-methylpropane-2-sulfinamide I-22d as a white solid and (S)—N—((S)-1-(2,3-dichloropyridin-4-yl)pent-4-en-1-yl)-2-methylpropane-2-sulfinamide P2 as a white solid.

I-22d: ¹H NMR (400 MHz, DMSO-d6) δ 8.40 (d, J=5.0 Hz, 1H), 7.59 (d, J=5.0 Hz, 1H), 5.90-5.74 (m, 2H), 5.17-4.96 (m, 2H), 4.65 (q, J=3.0 Hz, 1H), 2.23-2.11 (m, 2H), 1.95-1.67 (m, 2H), 1.07 (s, 9H).

P2: ¹H NMR (400 MHz, DMSO-d6) δ 8.39 (d, J=5.2 Hz, 1H), 7.68 (d, J=5.2 Hz, 1H), 6.08 (d, J=9.8 Hz, 1H), 5.85-5.74 (m, 1H), 5.11-4.93 (m, 2H), 4.64-4.58 (m, 1H), 2.24-2.02 (m, 2H), 1.90-1.60 (m, 2H), 1.12 (s, 9H).

Step 4: A mixture of (S)—N—((R)-1-(2,3-dichloropyridin-4-yl)pent-4-en-1-yl)-2-methylpropane-2-sulfinamide I-22d (500 mg, 1.49 mmol), Zn(CN)₂ (105 mg, 0.89 mmol), Zn (9.8 mg, 0.15 mmol) and Pd(dppf)Cl₂ (105 mg, 0.15 mmol) was heated at 160° C. in the microwave for 1 h. After completion of the reaction, the mixture was concentrated in vacuo and the residue was purified by silica gel column (50% EtOAc/petroleum ether) to give (S)—N—((R)-1-(3-chloro-2-cyanopyridin-4-yl)pent-4-en-1-yl)-2-methylpropane-2-sulfinamide I-22e as a yellow oil. LCMS (Method 5) m/z 326.1, 328.1 (M+H)⁺ (ES⁺), at 2.67 min

Step 5: (R)-4-(1-Aminopent-4-en-1-yl)-3-chloropicolinonitrile I-22f was synthesised from (S)—N—((R)-1-(3-chloro-2-cyanopyridin-4-yl)pent-4-en-1-yl)-2-methylpropane-2-sulfinamide I-22e using a procedure essentially the same as for I-71. LCMS (method 5) m/z 222.0, 224.0 (M+H)⁺ (ES⁺), at 0.55 min

Step 6: (R)-3-Chloro-4-(1-((4-methoxyphenyl)amino)pent-4-en-1-yl)picolinonitrile I-22g was synthesised from (R)-4-(1-aminopent-4-en-1-yl)-3-chloropicolinonitrile I-22f using a procedure essentially the same as I-7m. LCMS (Method 5) m/z 328.0, 330.0 (M+H)⁺ (ES⁺), at 1.69 min

Step 7: A mixture of (R)-3-chloro-4-(1-((4-methoxyphenyl)amino)pent-4-en-1-yl)picolino nitrile I-22g (100 mg, 0.31 mmol), NaO^{t}Bu (45 mg, 0.47 mmol) and Pd-172 (9.2 mg, 0.015 mmol) in toluene (1.5 ml) was heated at 95° C. for 2 h under a N₂ atmosphere. The mixture was allowed to cool to RT and concentrated in vacuo. The product was purified by prep-TLC (33% EtOAc/petroleum ether) to give (5R,8S)-10-(4-methoxyphenyl)-6,7,8,9-tetrahydro-5H-5,8-epiminocyclohepta[c]pyridine-1-carbonitrile I-22 h as a yellow solid. LCMS (Method 5) m/z 292.1 (M+H)⁺ (ES⁺), at 1.44 min

Step 8: (5R,8S)-6,7,8,9-Tetrahydro-5H-5,8-epiminocyclohepta[c]pyridine-1-carbonitrile I-22 was synthesised from (5R,8S)-10-(4-methoxyphenyl)-6,7,8,9-tetrahydro-5H-5,8-epiminocyclohepta[c]pyridine-1-carbonitrile I-22 h using a procedure essentially the same as 1-6. LCMS (Method 5) m/z 186.1 (M+H)⁺ (ES⁺), at 0.45 min

### Intermediate 23 (I-23)

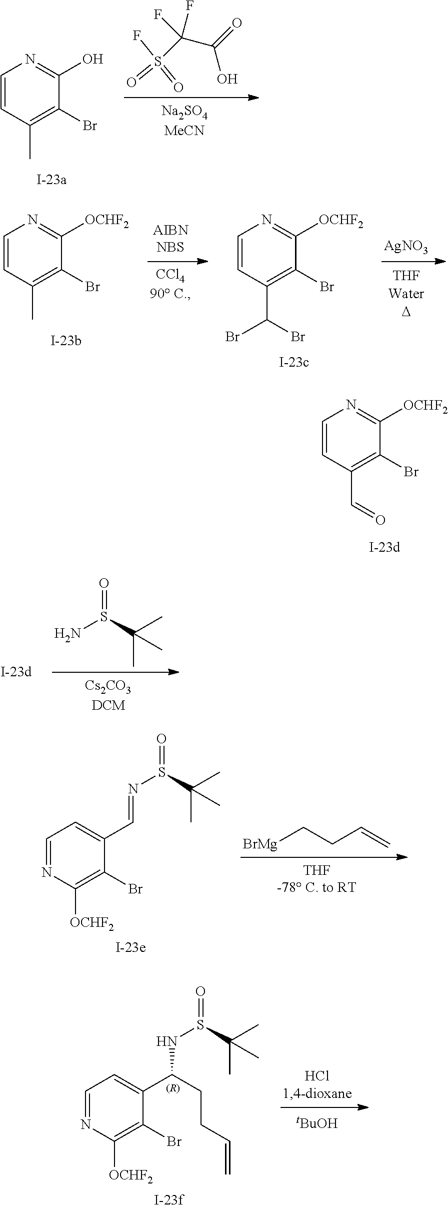

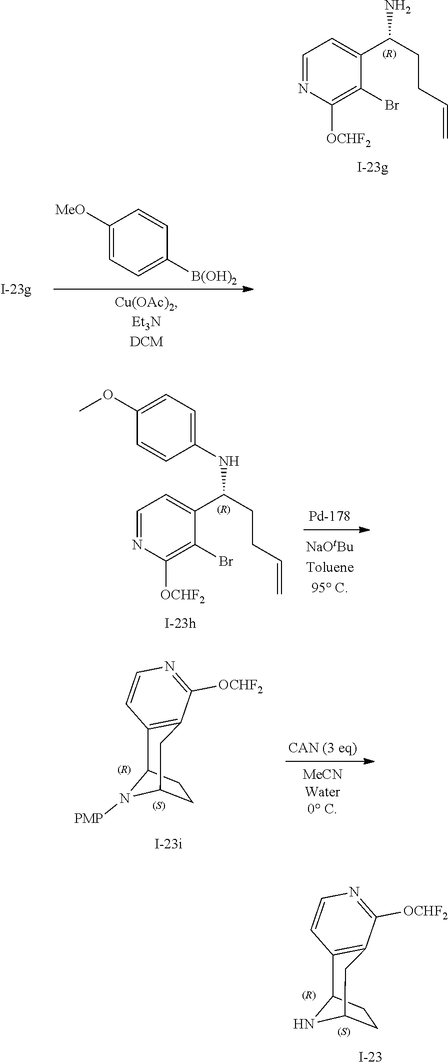

Step 1: To a solution of 3-bromo-4-methylpyridin-2-ol I-23a (4 g, 21.39 mmol) in MeCN (40 ml) was added 2,2-difluoro-2-(fluorosulfonyl)acetic acid (4.57 g, 25.67 mmol) and Na₂SO₄ (3.51 g, 24.72 mmol). The mixture was stirred at RT for 16 h. The mixture was then diluted with water (40 ml) and the product was extracted with DCM (2×40 ml). The combined organics were washed with brine, dried over Na₂SO₄ and concentrated in vacuo. The residue was purified by chromatography on silica gel (5% EtOAc/petroleum ether) to give 3-bromo-2-(difluoromethoxy)-4-methylpyridine I-23b as a colorless oil. LCMS (Method 5) m/z 238 (M+H)⁺ (ES⁺), at 0.45 min. ¹H NMR (400 MHz, CDCl₃) δ 7.96 (d, J=5.2 Hz, 1H), 7.44 (t, J=72.4 Hz, 1H), 6.98 (d, J=5.2 Hz, 1H), 2.45 (s, 3H).

Step 2: To a solution of 3-bromo-2-(difluoromethoxy)-4-methylpyridine I-23b (4 g, 16.88 mmol) in CCl₄ (40 ml) was added AlBN (284 mg, 1.68 mmol) and NBS (9.24 g, 50.63 mmol). The reaction was heated at 90° C. for 16 h. The reaction mixture was concentrated in vacuo and purified by chromatography on silica gel (5-10% EtOAc/petroleum ether) to give 3-bromo-4-(dibromomethyl)-2-(difluoromethoxy)pyridine I-23c as a yellow solid. ¹H NMR (400 MHz, DMSO-d6) δ 8.35 (d, J=5.6 Hz, 1H), 7.72 (t, J=72.0 Hz, 1H), 7.77 (d, J=5.2 Hz, 1H), 7.34 (s, 1H).

Step 3: To a solution of 3-bromo-4-(dibromomethyl)-2-(difluoromethoxy)pyridine I-23c (5 g, 12.72 mmol) in a mixture of THF (50 ml) and water (10 ml) was added AgNO₃ (7.57 g, 44.53 mmol). The resulting mixture was heated at reflux for 16 h then diluted with water (70 ml). The product was extracted with DCM (2×100 ml) and the combined organics were washed with brine, dried over Na₂SO₄ and concentrated in vacuo. The residue was purified by silica gel column (2% EtOAc/petroleum ether) to give 3-bromo-2-difluoromethoxy)isonicotinaldehyde I-23d as a yellow solid. ¹H NMR (400 MHz, DMSO-d6) δ 10.22 (s, 1H), 8.41 (d, J=4.8 Hz, 1H), 7.77 (t, J=72.0 Hz, 1H), 7.55 (d, J=5.2 Hz, 1H).

Step 4: (S)—N-((3-Bromo-2-(difluoromethoxy)pyridin-4-yl)methylene)-2-methylpropane-2-sulfinamide I-23e was synthesised from 3-bromo-2-difluoromethoxy)isonicotinaldehyde I-23d using a procedure essentially the same as for I-7j. ¹H NMR (400 MHz, DMSO-d6) δ 8.79 (s, 1H), 8.35 (d, J=12.0 Hz, 1H), 7.76 (t, J=71.6 Hz, 1H), 7.74 (d, J=4.8 Hz, 1H), 1.22 (s, 9H).

Step 5: To a solution of (S)—N-((3-bromo-2-(difluoromethoxy)pyridin-4-yl)methylene)-2-methylpropane-2-sulfinamide I-23e (2 g, 5.65 mmol) in THF (20 ml) at −70° C. was added a solution of but-3-en-1-ylmagnesium bromide (17 ml, 0.5 M in THF, 8.47 mmol) dropwise. The resulting solution was stirred at −70° C. for 1 h. Aqueous NH₄Cl solution was added to the reaction mixture. The mixture was diluted with water (20 ml) and the aqueous layer extracted with DCM (2×20 ml). The combined organics were washed with brine, dried over Na₂SO₄ and concentrated in vacuo. The product was purified by chromatography on silica gel column (10% MeOH/DCM) to give (S)—N—((R)-1-(3-bromo-2-(difluoromethoxy)pyridin-4-yl)pent-4-en-1-yl)-2-methylpropane-2-sulfinamide I-23f as a yellow oil. ¹H NMR (400 MHz, DMSO-d6) δ 8.23 (d, J=5.2 Hz, 1H), 7.71 (t, J=72.4 Hz, 1H), 7.40 (d, J=5.2 Hz, 1H), 5.89-5.78 (m, 2H), 5.13-5.00 (m, 2H), 4.68-4.63 (m, 1H), 2.27-2.08 (m, 2H), 1.92-1.69 (m, 2H), 1.01 (s, 9H).

Step 6: To a solution of (S)—N—((R)-1-(3-bromo-2-(difluoromethoxy)pyridin-4-yl)pent-4-en-1-yl)-2-methylpropane-2-sulfinamide I-23f (1.5 g, 3.66 mmol) in ^{t}BuOH (15 ml) at 0° C. was added a solution of HCl in dioxane (4.57 ml, 4 M, 18.29 mmol). The reaction was stirred at RT for 2.5 h then water was added. The pH of the aqueous layer adjusted to 7-8 by addition of saturated aqueous NaHCO₃. The aqueous layer was extracted with DCM (2×30 ml) and the combined organics were washed with brine, dried over Na₂SO₄ and concentrated in vacuo to give (R)-1-(3-bromo-2-(difluoromethoxy)pyridin-4-yl)pent-4-en-1-amine I-23f as a yellow oil. LCMS (Method 5) m/z 306.9, 308.9 (M+H)⁺ (ES⁺), at 1.06 min

Step 7: (R)—N-(1-(3-Bromo-2-(difluoromethoxy)pyridine-4-yl)pent-4-en-1-yl)-4-methoxyaniline I-23g was synthesised from (R)-1-(3-bromo-2-(difluoromethoxy)pyridine-4-yl)pent-4-en-1-amine I-23f using a procedure essentially the same as for I-7m. ¹H NMR (400 MHz, DMSO-d6) δ 8.11 (d, J=5.2 Hz, 1H), 7.71 (t, J=72.0 Hz, 1H), 7.28 (d, J=5.2 Hz, 1H), 6.64 (d, J=8.8 Hz, 2H), 6.36 (d, J=9.2 Hz, 2H), 6.11 (d, J=8.0 Hz, 1H), 5.91-5.81 (m, 1H), 5.06-4.98 (m, 2H), 4.66-4.60 (m, 1H), 3.57 (s, 3H), 2.34-2.15 (m, 2H), 1.78-1.72 (m, 2H).

Step 8: (5R,8S)-1-(Difluoromethoxy)-10-(4-methoxyphenyl)-6,7,8,9-tetrahydro-5H-5,8-epiminocyclohepta[c]pyridine I-23 h was synthesised from (R)—N-(1-(3-bromo-2-(difluoromethoxy)pyridine-4-yl)pent-4-en-1-yl)-4-methoxyaniline I-23g using a procedure essentially the same as for I-20 h. LCMS (Method 5) m/z 333.0 (M+H)⁺ (ES⁺), at 1.61 min. ¹H NMR (400 MHz, DMSO-d6) δ 7.97 (d, J=5.2 Hz, 1H), 7.59 (t, J=73.2 Hz, 1H), 7.20 (d, J=4.8 Hz, 1H), 6.80 (d, J=9.2 Hz, 2H), 6.70 (d, J=9.2 Hz, 2H), 4.89 (d, J=5.2 Hz, 1H), 4.54 (t, J=5.2 Hz, 1H), 3.61 (s, 3H), 2.88 (dd, J=4.8 Hz, 18.0 Hz, 1H), 2.30-2.26 (m, 3H), 1.86-1.74 (m, 2H).

Step 9: (5R,8S)-1-(Difluoromethoxy)-6,7,8,9-tetrahydro-5H-5,8-epiminocyclohepta[c]pyridine I-23 was synthesized from (5R,8S)-1-(difluoromethoxy)-10-(4-methoxyphenyl)-6,7,8,9-tetrahydro-5H-5,8-epiminocyclohepta[c]pyridine I-23 h using a procedure essentially the same as I-6. LCMS (Method 5) m/z 227.0 (M+H)⁺ (ES⁺), at 0.24 min

### Intermediate 25 (I-25)

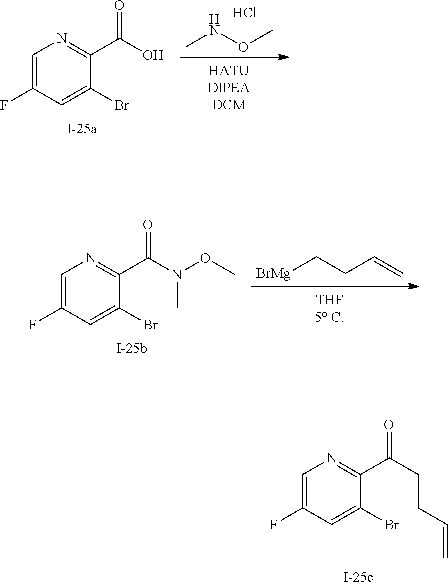

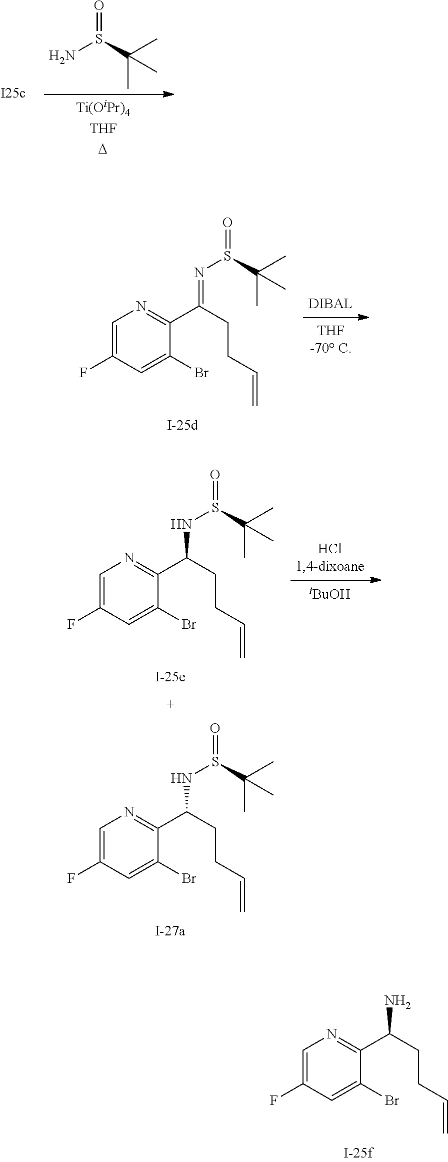

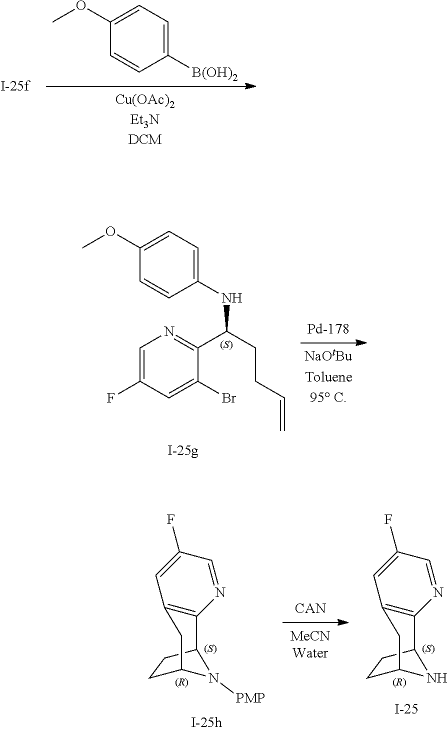

Step 1: To a solution of 3-bromo-5-fluoropicolinic acid I-25a (5 g, 22.72 mmol) in DCM (100 ml) at 0° C. were added N,O-dimethylhydroxylamine hydrochloride (2.43 g, 25 mmol), DIPEA (15.86 ml, 90.88 mmol) and HATU (12.95 g, 34.08 mmol). The reaction mixture was stirred at RT for 16 h. The reaction mixture was diluted with DCM (50 ml) and washed with water (2×100 ml). The combined organics were dried over Na₂SO₄ and concentrated in vacuo. The product was purified by chromatography on silica gel (20% EtOAc/petroleum ether) to 3-bromo-5-fluoro-N-methoxy-N-methylpicolinamide as an oil. ¹H NMR (400 MHz, DMSO-d6) δ 8.66 (d, J=2.4 Hz, 1H), 8.33 (dd, J=2.4, 8.8 Hz, 1H), 3.48 (s, 3H), 3.30 (s, 3H).

Step 2: To a solution of 3-bromo-5-fluoro-N-methoxy-N-methylpicolinamide (4.2 g, 16.03 mmol) in THF (80 ml) at 0° C. was added dropwise a solution of but-3-en-1-ylmagnesium bromide (48 ml, 0.5 M in THF, 24 mmol). The reaction was stirred for 30 min then poured into saturated aqueous NH₄Cl (50 ml) and the product was extracted with EtOAc (4×50 ml). The combined organics were washed with brine (50 ml), dried over Na₂SO₄ and concentrated in vacuo. The product was purified by chromatography on silica gel (5% EtOAc/petroleum ether) to give 1-(3-bromo-5-fluoropyridin-2-yl)pent-4-en-1-one I-25c as a yellow oil. ¹H NMR (400 MHz, CDCl₃) δ 8.44 (d, J=2 Hz, 1H), 7.75 (dd, J=2.4 Hz, 7.6 Hz, 1H), 5.93-5.83 (m, 1H), 5.10-4.99 (m, 2H), 3.19 (t, J=7.2 Hz, 2H), 2.49-2.44 (m, 2H).

Step 3: (S)—N-(1-(3-Bromo-5-fluoropyridin-2-yl)pent-4-en-1-ylidene)-2-methylpropane-2-sulfinamide I-25d was synthesised from 1-(3-bromo-5-fluoropyridin-2-yl)pent-4-en-1-one I-25c using a procedure essentially the same as for I-20d. ¹H NMR (400 MHz, CDCl₃) 8.40 (s, 1H), 7.61 (d, J=6.6 Hz, 1H), 5.96-5.83 (m, 1H), 5.06 (dd, J=31.8, 13.8 Hz, 2H), 2.97-2.89 (m, 1H), 2.76-2.68 (m, 1H), 2.51-2.46 (m, 2H), 1.26 (s, 9H).

Step 4: To a solution of (S)—N-(1-(3-bromo-5-fluoropyridin-2-yl)pent-4-en-1-ylidene)-2-methylpropane-2-sulfinamide I-25d (1.85 g, 5.13 mmol) in THF (18 ml) at −70° C. under N₂ atmosphere was added a solution of DIBAL (7.7 ml, 1 M in THF, 7.7 mmol) dropwise. The reaction mixture was stirred for 2 h at −70° C., then quenched with MeOH (5 ml) and the mixture was concentrated in vacuo. The residue was diluted with aqueous NaOH solution (1 M, 50 ml) and the product was extracted with EtOAc (3×100 ml). The combined organics were washed with brine, dried over Na₂SO₄ and concentrated in vacuo. The product was purified by reverse phase chromatography (40% MeCN/water) to give (S)—N—((S)-1-(3-bromo-5-fluoropyridin-2-yl)pent-4-en-1-yl)-2-methylpropane-2-sulfinamide I-25e as a yellow oil. Further elution provided the diastereomer (S)—N—((R)-1-(3-bromo-5-fluoropyridin-2-yl)pent-4-en-1-yl)-2-methylpropane-2-sulfinamide I-27a

(S)—N—((S)-1-(3-bromo-5-fluoropyridin-2-yl)pent-4-en-1-yl)-2-methylpropane-2-sulfinamide I-25e ¹H NMR (400 MHz, DMSO-d6) δ 8.66-8.64 (m, 1H), 8.24-8.18 (m, 1H), 5.86-5.71 (m, 1H), 5.40 (d, J=9.4 Hz, 1H), 5.07-4.95 (m, 3H), 4.72 (d, J=6.8 Hz, 2H), 2.17-2.03 (m, 3H), 1.80-1.75 (m, 2H), 1.12 (s, 9H).

(S)—N—((R)-1-(3-bromo-5-fluoropyridin-2-yl)pent-4-en-1-yl)-2-methylpropane-2-sulfinamide I-27a ¹H NMR (400 MHz, DMSO-d6) δ 8.63 (d, J=2.4 Hz, 1H), 8.14 (dd, J=2.4, 8.4 Hz, 1H), 5.83-5.72 (m, 1H), 5.03-4.95 (m, 2H), 4.82-4.79 (m, 1H), 2.12-1.95 (m, 5H), 1.03 (s, 9H).

Step 5: (S)-1-(3-Bromo-5-fluoropyridin-2-yl)pent-4-en-1-amine I-25f was synthesised from (S)—N—((S)-1-(3-bromo-5-fluoropyridin-2-yl)pent-4-en-1-yl)-2-methylpropane-2-sulfinamide I-25e using a procedure essentially the same as I-71. LCMS (Method 3) m/z 259.2, 261.2 (M+H)⁺ (ES⁺), at 0.27 min

Step 6: (S)—N-(1-(3-Bromo-5-fluoropyridin-2-yl)pent-4-en-1-yl)-4-methoxyaniline I-25g was synthesized from (S)-1-(3-bromo-5-fluoropyridin-2-yl)pent-4-en-1-amine I-25f using a procedure essentially the same as I-7m. ¹H NMR (400 MHz, CDCl₃) δ 8.37 (d, J=2.6 Hz, 1H), 7.57 (dd, J=7.6, 2.4 Hz, 1H), 6.71-6.69 (m, 2H), 6.62-6.60 (m, 2H), 5.88-5.78 (m, 1H), 5.05-4.94 (m, 3H), 3.70 (s, 3H), 2.32-2.10 (m, 2H), 1.90-1.81 (m, 2H).

Step 7: (6R,9S)-3-Fluoro-10-(4-methoxyphenyl)-6,7,8,9-tetrahydro-5H-6,9-epiminocyclohepta[b]pyridine I-25 h was synthesised from (S)—N-(1-(3-bromo-5-fluoropyridin-2-yl)pent-4-en-1-yl)-4-methoxyaniline I-25g using a procedure essentially the same as I-20 h. ¹H NMR (400 MHz, CDCl₃) δ 8.18 (d, J=2.6 Hz, 1H), 6.97-6.95 (m, 1H), 6.77-6.70 (m, 4H), 4.80 (d, J=6 Hz, 1H), 4.45 (t, J=4.8 Hz, 1H), 3.69 (s, 3H), 3.28 (dd, J=17.2, 4.6 Hz, 1H), 2.43-2.39 (m, 3H), 2.00-1.96 (m, 1H), 1.78-1.71 (m, 1H).

Step 8: (6R,9S)-3-Fluoro-6,7,8,9-tetrahydro-5H-6,9-epiminocyclohepta[b]pyridine I-25 was synthesised from (6R,9S)-3-fluoro-10-(4-methoxyphenyl)-6,7,8,9-tetrahydro-5H-6,9-epiminocyclohepta[b]pyridine I-25 h using a procedure essentially the same as for I-6. LCMS (Method 3) m/z 179.1 (M+H)⁺ (ES⁺), at 1.36 min

### Intermediate 26 (I-26)

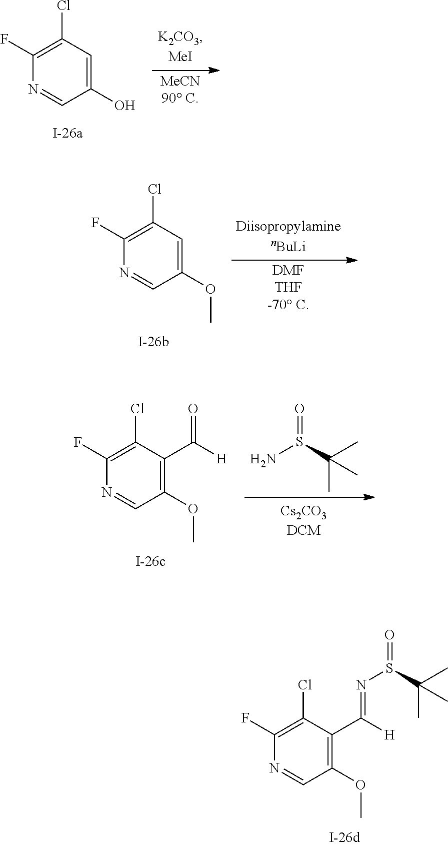

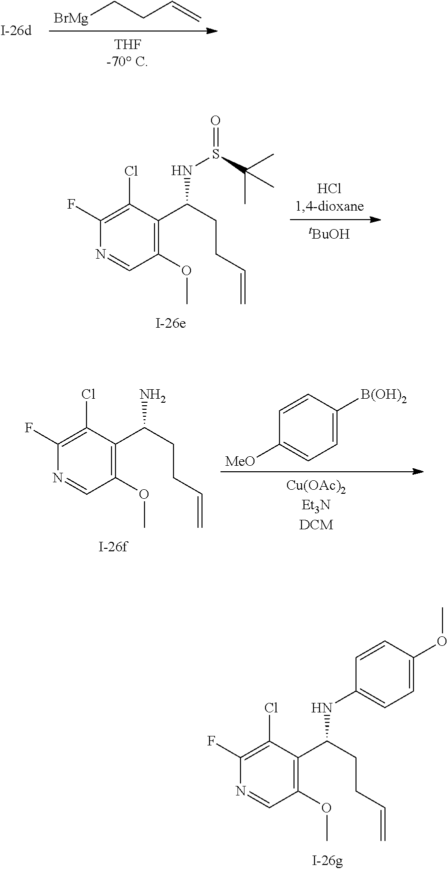

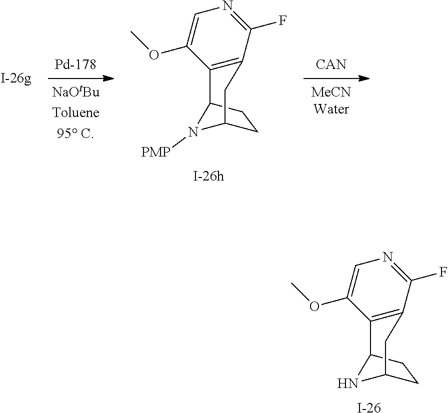

Step 1: To a solution of 5-chloro-6-fluoropyridin-3-ol I-26a (2.8 g, 19.05 mmol) in MeCN (28 ml) was added potassium carbonate (7.9 g, 57.15 mmol) and Mel (3.24 g, 22.86 mmol). The reaction mixture was heated at 90° C. in a sealed tube overnight. After cooling to RT, the mixture was diluted with water (50 ml) and the product was extracted with EtOAc (3×100 ml). The combined organics were dried over Na₂SO₄ and concentrated in vacuo. The product was purified by chromatography on silica gel (10% EtOAc/petroleum ether) to give 3-chloro-2-fluoro-5-methoxypyridine as a white solid. ¹H NMR (400 MHz, CDCl₃) δ 7.72 (t, J=2.4 Hz, 1H), 7.37 (dd, J=2.8, 7.2 Hz, 1H), 3.85 (s, 3H).

Step 2: To a solution of diisopropylamine (2.6 ml, 18.63 mmol) in THF (15 ml) at −70° C. was added dropwise ⁿBuLi in hexanes (7.45 ml, 2.5 M, 18.63 mmol). The reaction mixture was stirred at −70° C. for 1 h then a solution of 3-chloro-2-fluoro-5-methoxypyridine I-26b (2 g, 12.42 mmol) in THF (5 ml) was added and the mixture stirred for a further 30 min. DMF (1.44 ml, 18.63 mmol) was added and the reaction stirred for another 30 min. The reaction mixture was poured into a saturated NH₄Cl solution and the product was extracted with EtOAc (4×50 ml). The combined organics were washed with brine, dried over Na₂SO₄ and concentrated in vacuo. The product was purified by chromatography on silica gel (20% EtOAc/petroleum ether) to give 3-chloro-2-fluoro-5-methoxyisonicotinaldehyde I-26c as a yellow oil. ¹H NMR (400 MHz, CDCl₃) δ 10.42 (s, 1H), 7.88 (s, 1H), 4.01 (s, 3H).

Step 3: (S)—N-((3-Chloro-2-fluoro-5-methoxypyridin-4-yl)methylene)-2-methylpropane-2-sulfinamide 126d was synthesized from 3-chloro-2-fluoro-5-methoxyisonicotinaldehyde I-26c using a procedure essentially the same as for I-7j. ¹H NMR (400 MHz, CDCl₃) δ 8.83 (s, 1H), 7.81 (s, 1H), 3.96 (s, 3H), 1.29 (s, 9H).

Step 4: (S)—N—((R)-1-(3-Chloro-2-fluoro-5-methoxypyridin-4-yl)pent-4-en-1-yl)-2-methylpropane-2-sulfinamide I-26e was synthesised from (S)—N-((3-chloro-2-fluoro-5-methoxypyridin-4-yl)methylene)-2-methylpropane-2-sulfinamide 126d using a procedure essentially the same as for I-7k. LCMS (Method 3) m/z 349.1 (M+H)⁺ (ES⁺), at 1.69 min

Step 5: (R)-1-(3-Chloro-2-fluoro-5-methoxypyridin-4-yl)pent-4-en-1-amine I-26f was synthesised from (S)—N-(I-1-(3-chloro-2-fluoro-5-methoxypyridin-4-yl)pent-4-en-1-yl)-2-methylpropane-2-sulfinamide I-26e using a procedure essentially the same as for I-71. LCMS (Method 3) m/z 245.1, 247.1 (M+H)⁺ (ES⁺), at 0.69 min

Step 6: (R)—N-(1-(3-Chloro-2-fluoro-5-methoxypyridin-4-yl)pent-4-en-1-yl)-4-methoxyaniline I-26g was synthesised from (R)-1-(3-chloro-2-fluoro-5-methoxypyridin-4-yl)pent-4-en-1-amine I-26f using a procedure essentially the same as for I-7m. ¹H NMR (400 MHz, CDCl₃) δ 7.59 (s, 1H), 6.71-6.67 (m, 2H), 6.58-6.55 (m, 2H), 5.87-5.77 (m, 1H), 5.06-4.99 (s, 3H), 4.40 (br. s, 1H), 3.95 (s, 3H), 3.69 (s, 3H), 2.32-2.26 (m, 1H), 2.15-2.09 (m, 2H), 1.93-1.87 (m, 1H).

Step 7: (5R,8S)-1-Fluoro-4-methoxy-10-(4-methoxyphenyl)-6,7,8,9-tetrahydro-5H-5,8-epiminocyclohepta[c]pyridine I-26 h was synthesised from (R)—N-(1-(3-chloro-2-fluoro-5-methoxypyridin-4-yl)pent-4-en-1-yl)-4-methoxyaniline I-26g using a procedure essentially the same as for I-20 h. ¹H NMR (400 MHz, CDCl₃) δ 7.53 (s, 1H), 6.74-6.68 (m, 4H), 5.03 (d, J=6 Hz, 1H), 4.48 (t, J=5.6 Hz, 1H), 3.94 (s, 3H), 3.70 (s, 3H), 3.06-3.00 (m, 1H), 2.46-2.29 (m, 3H), 1.93-1.88 (m, 1H), 1.79-1.72 (m, 1H).

Step 8: (5R,8S)-1-Fluoro-4-methoxy-6,7,8,9-tetrahydro-5H-5,8-epiminocyclohepta [c]pyridine I-26 was synthesised from (5R,8S)-1-fluoro-4-methoxy-10-(4-methoxyphenyl)-6,7,8,9-tetrahydro-5H-5,8-epiminocyclohepta[c]pyridine I-26 h using a procedure essentially the same as for I-6. LCMS (Method 3) m/z 209.1 (M+H)⁺ (ES⁺), at 0.28 min

### Intermediate 27 (I-27)

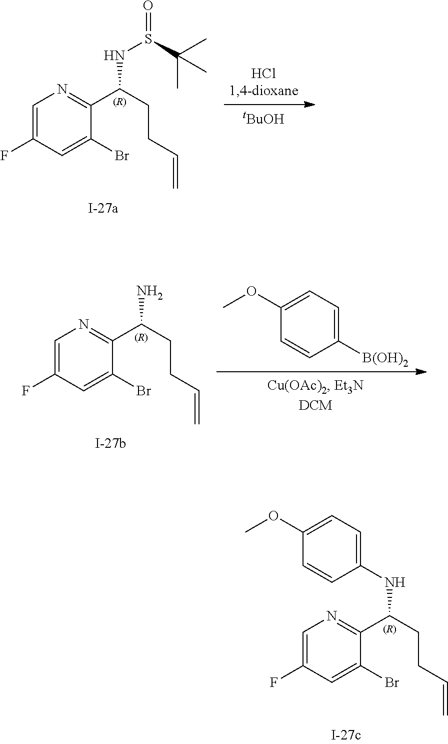

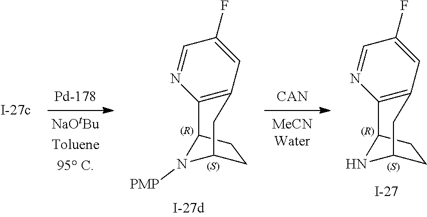

Step 1: (R)-1-(3-Bromo-5-fluoropyridin-2-yl)pent-4-en-1-amine I-27b was synthesised from (S)—N—((R)-1-(3-bromo-5-fluoropyridin-2-yl)pent-4-en-1-yl)-2-methyl propane-2-sulfinamide I-27a using a procedure essentially the same as for I-71. LCMS (Method 3) m/z 259.2, 261.2 (M+H)⁺ (ES⁺), at 0.27 min

Step 2: (R)—N-(1-(3-Bromo-5-fluoropyridin-2-yl)pent-4-en-1-yl)-4-methoxyaniline I-27c was synthesised from (R)-1-(3-bromo-5-fluoropyridin-2-yl)pent-4-en-1-amine I-27b using a procedure essentially the same as for I-7m. ¹H NMR (400 MHz, methanol-d4) δ 8.43 (d, J=2.4 Hz, 1H), 7.84 (dd, J=8.0, 2.4 Hz, 1H), 6.66-6.59 (m, 4H), 5.89-5.79 (m, 1H), 5.04-4.95 (m, 3H), 3.64 (s, 3H), 2.28-2.22 (m, 1H), 2.17-2.10 (m, 1H), 1.89-1.83 (m, 2H).

Step 3: (6S,9R)-3-Fluoro-10-(4-methoxyphenyl)-6,7,8,9-tetrahydro-5H-6,9-epiminocyclohepta[b]pyridine I-27d was synthesised from (R)—N-(1-(3-bromo-5-fluoropyridin-2-yl)pent-4-en-1-yl)-4-methoxyaniline I-27c using a procedure essentially the same as for I-20 h. ¹H NMR (400 MHz, Methanol-d4) δ 8.16 (d, J=2.4 Hz, 1H), 7.23 (dd, J=2.4, 8.8, 1H), 6.79-6.70 (m, 4H), 4.76 (d, J=6 Hz, 1H), 4.55-4.48 (m, 2H), 3.66 (s, 3H), 2.41-2.34 (m, 2H), 1.97-1.92 (m, 1H), 1.82-1.77 (m, 1H).

Step 4: (6S,9R)-3-Fluoro-6,7,8,9-tetrahydro-5H-6,9-epiminocyclohepta[b]pyridine I-27 was synthesised from (6S,9R)-3-fluoro-10-(4-methoxyphenyl)-6,7,8,9-tetrahydro-5H-6,9-epiminocyclohepta[b]pyridine I-27d using a procedure essentially the same as for I-6. LCMS (Method 3) m/z 179.1 (M+H)⁺ (ES⁺), at 0.35 min

### Intermediate 28 (I-28)

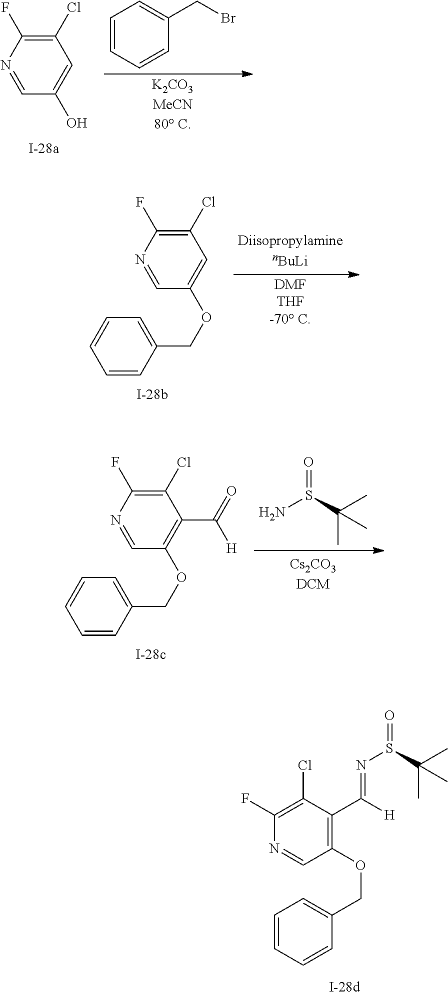

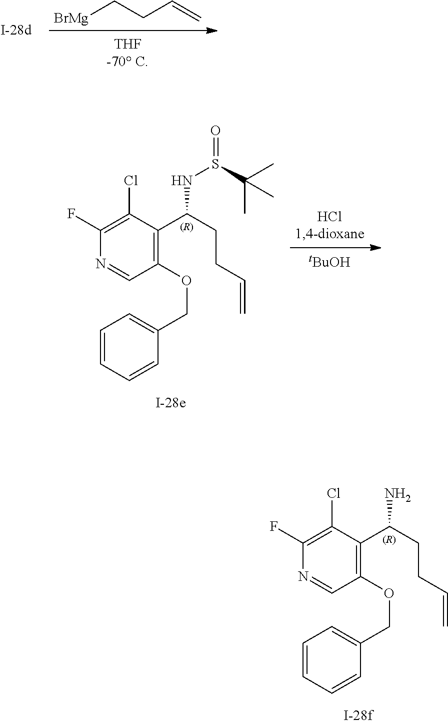

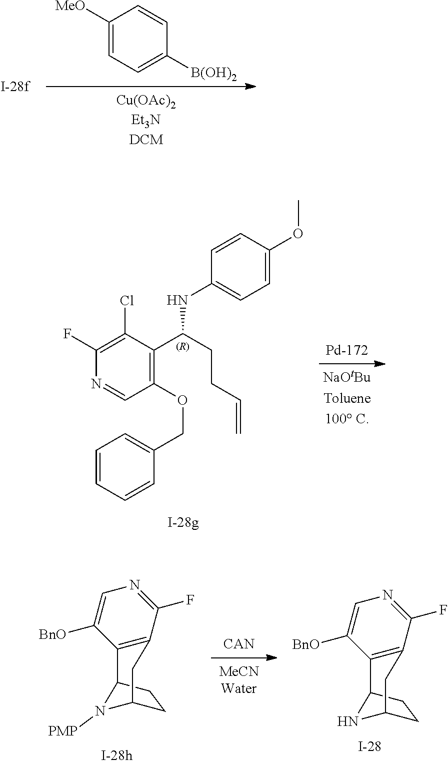

Step 1: To a solution of 5-chloro-6-fluoropyridin-3-ol I-28a (10 g, 67.8 mmol) and K₂CO₃ (28.1 g, 203.4 mmol) in MeCN was added benzyl bromide (13.9 g, 81.4 mmol). The reaction was heated at 80° C. for 3 h. The reaction mixture was diluted with water and the product was extracted with EtOAc (3×50 ml). The combined organics were washed with brine, dried over Na₂SO₄ and concentrated in vacuo. The product was purified by chromatography on silica gel (10% EtOAc/petroleum ether) to afford 5-(benzyloxy)-3-chloro-2-fluoropyridine I-28b as a white solid. ¹H NMR (400 MHz, DMSO-d6) δ 8.00-7.95 (m, 2H), 7.47-7.35 (m, 5H), 5.20 (s, 2H).

Step 2: A solution of diisopropylamine (11.6 ml, 82.1 mmol) in THF was cooled to −70° C. and a solution of ⁿBuLi in hexanes (32.8 ml, 2.5 M, 82.1 mmol) was added dropwise. The reaction mixture was stirred at −70° C. for 1 h then a solution of 5-(benzyloxy)-3-chloro-2-fluoropyridine I-28b (13 g, 54.7 mmol) in THF (80 ml) was added. After stirring at −70° C. for 0.5 h, DMF (6.35 ml, 82.1 mmol) was added and the reaction stirred for a further hour at −70° C. The reaction was poured onto saturated aqueous NH₄Cl solution (100 ml) and the product was extracted with EtOAc (3×100 ml). The combined organics were washed with brine, dried over Na₂SO₄ and concentrated in vacuo. The product was purified by chromatography on silica gel (10% EtOAc/petroleum ether) to give 5-(benzyloxy)-3-chloro-2-fluoroisonicotinaldehyde I-28c as a white solid. ¹H NMR (400 MHz, DMSO-d6) δ 10.32 (d, J=1.2 Hz, 1H), 8.28 (d, J=2.0 Hz, 1H), 7.50-7.48 (m, 2H), 7.43-7.34 (m, 3H), 5.36 (s, 2H).

Step 3: (S)—N-((5-(Benzyloxy)-3-chloro-2-fluoropyridin-4-yl)methylene)-2-methylpropane-2-sulfinamide I-28d was synthesised from 5-(benzyloxy)-3-chloro-2-fluoroisonicotinaldehyde I-28c using a procedure essentially the same as for I-7j. LCMS (Method 5) m/z 369.1 (M+H)⁺ (ES⁺), at 1.72 min

Step 4: (S)—N—((R)-1-(5-(Benzyloxy)-3-chloro-2-fluoropyridin-4-yl)pent-4-en-1-yl)-2-methylpropane-2-sulfinamide I-28e was synthesised from (S)—N-((5-(benzyloxy)-3-chloro-2-fluoropyridin-4-yl)methylene)-2-methylpropane-2-sulfinamide I-28d using a procedure essentially the same as for I-7k. LCMS (Method 5) m/z 425.1 (M+H)⁺ (ES⁺), at 1.75 min

Step 5: (R)-1-(5-(Benzyloxy)-3-chloro-2-fluoropyridin-4-yl)pent-4-en-1-amine I-28f was synthesised from (S)—N—((R)-1-(5-(benzyloxy)-3-chloro-2-fluoropyridin-4-yl)pent-4-en-1-yl)-2-methylpropane-2-sulfinamide I-28e using a procedure essentially the same as for I-71. LCMS (Method 5) m/z 321.1 (M+H)⁺ (ES⁺), at 1.25 min

Step 6: (R)—N-(1-(5-(Benzyloxy)-3-chloro-2-fluoropyridin-4-yl)pent-4-en-1-yl)-4-methoxyaniline I-28g was synthesised from (R)-1-(5-(benzyloxy)-3-chloro-2-fluoropyridin-4-yl)pent-4-en-1-amine I-28f using a procedure essentially the same as for I-7m. ¹H NMR (400 MHz, DMSO-d6) δ 7.94 (s, 1H), 7.51 (d, J=7.4 Hz, 1H), 7.44-7.35 (m, 3H), 6.63 (d, J=8.3 Hz, 2H), 6.46 (d, J=8.4 Hz, 2H), 5.83-5.73 (m, 1H), 5.33 (s, 2H), 5.00-4.92 (m, 3H), 4.80-4.74 (m, 1H), 3.58 (s, 3H), 2.27-1.97 (m, 3H), 1.95-1.82 (m, 1H).

Step 7: (5R,8S)-4-(Benzyloxy)-1-fluoro-10-(4-methoxyphenyl)-6,7,8,9-tetrahydro-5H-5,8-epiminocyclohepta[c]pyridine I-28 h was synthesised from (R)—N-(1-(5-(benzyloxy)-3-chloro-2-fluoropyridin-4-yl)pent-4-en-1-yl)-4-methoxyaniline I-28g using a procedure essentially the same as for I-22 h. ¹H NMR (400 MHz, DMSO-d6) δ 7.78 (s, 1H), 7.54 (d, J=7.1 Hz, 2H), 7.46 (t, J=15.2 Hz, 2H), 7.39-7.35 (m, 1H), 6.71-6.65 (m, 4H), 5.34-5.27 (m, 2H), 5.01 (d, J=5.4 Hz, 1H), 4.54 (t, J=5.6 Hz, 1H), 3.61 (s, 3H), 2.88 (dd, J=17.7, 4.8 Hz, 1H), 2.32-2.23 (m, 3H), 1.84-1.74 (m, 2H).

Step 8: (5R,8S)-4-(benzyloxy)-1-fluoro-6,7,8,9-tetrahydro-5H-5,8-epiminocyclohepta[c]pyridine I-28 was synthesised from (5R,8S)-4-(benzyloxy)-1-fluoro-10-(4-methoxyphenyl)-6,7,8,9-tetrahydro-5H-5,8-epiminocyclohepta[c]pyridine I-28 h using a procedure essentially the same as for I-6. LCMS (Method 5) m/z 285.1 (M+H)⁺ (ES⁺), at 1.07 min

### Intermediate 29 (I-29)

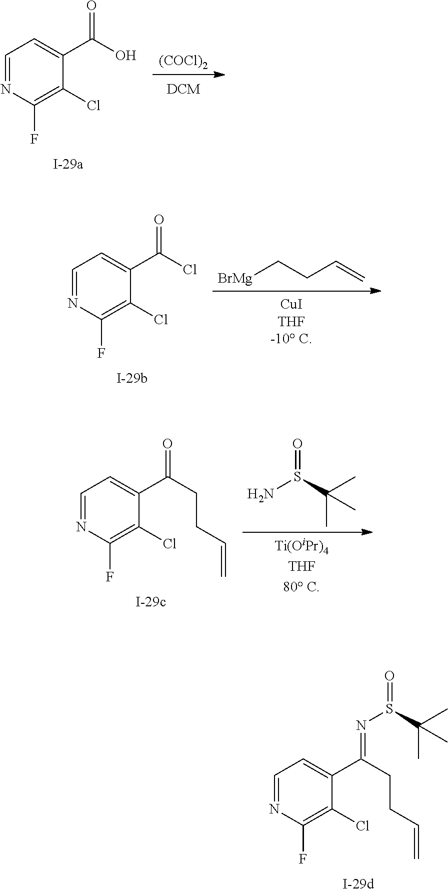

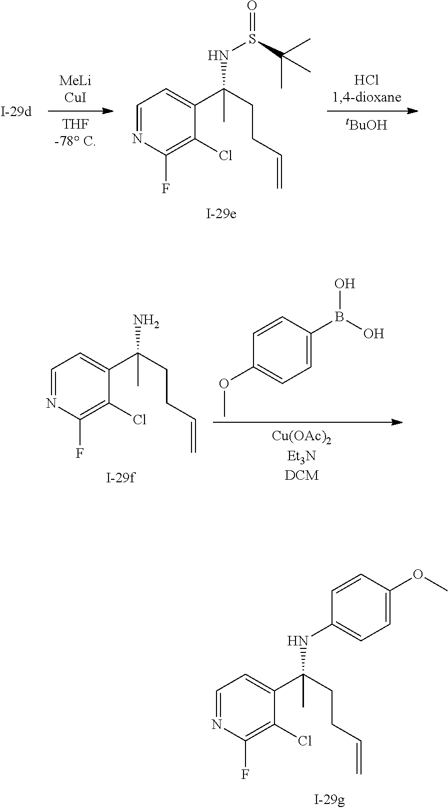

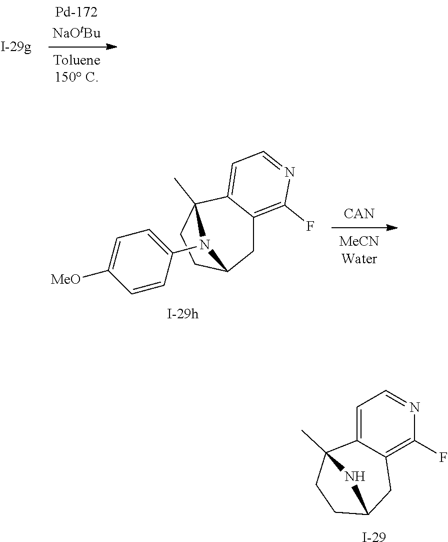

Step 1: To a solution of 3-chloro-2-fluoroisonicotinic acid I-29a (1 g, 5.7 mmol) in DCM (10 ml) at 0° C. was added a drop of DMF and oxalyl chloride (2.3 g, 18.2 mol). The mixture was stirred at RT for 2 h, concentrated in vacuo to give 3-chloro-2-fluoroisonicotinoyl chloride I-29b which was used in the next step without any further purification or analysis.

Step 2: To a solution of 3-chloro-2-fluoroisonicotinoyl chloride I-29b in THF (10 ml) was added CuI (54 mg, 0.28 mol) and a solution of but-3-en-1-ylmagnesium bromide (25 ml, 0.5 M in THF, 12.5 mmol) in THF (8 ml) slowly at −78° C., the resulting mixture was stirred at −78° C. for 2 h. The reaction was poured onto saturated aqueous NH₄Cl solution (10 ml) and the product was extracted with EtOAc (3×10 ml). The combined organics were washed with brine, dried over Na₂SO₄ and concentrated in vacuo. The product was purified by chromatography on silica gel (2% MeOH/DCM) to 1-(3-chloro-2-fluoropyridin-4-yl)pent-4-en-1-one I-29c as a light yellow oil. ¹H NMR (400 MHz, DMSO-d6) δ 8.32 (dd, J=0.8, 4.8 Hz, 1H), 7.64 (d, J=4.8 Hz, 1H), 5.89-5.79 (m, 1H), 5.10-4.98 (m, 2H), 3.08 (t, J=7.2 Hz, 2H), 2.39-2.34 (m, 2H).

Step 3: To a solution of 1-(3-chloro-2-fluoropyridin-4-yl)pent-4-en-1-one I-29c (2 g, 9.4 mmol) in THF (30 ml) was added (S)-2-methylpropane-2-sulfinamide (3.4 g, 28.1 mmol) and Ti(OⁱPr)₄ (13.3 g, 46.8 mmol) at RT. The reaction mixture was heated at 80° C. for 16 h. The mixture was poured into water and the product was extracted with EtOAc (2×100 ml). The combined organics were washed with brine, dried over Na₂SO₄ and concentrated in vacuo. The product was purified by chromatography on silica gel (10-20% EtOAc/petroleum ether) to give (S)—N-(1-(3-chloro-2-fluoropyridin-4-yl)pent-4-en-1-ylidene)-2-methylpropane-2-sulfinamide I-29d as a yellow solid. ¹H NMR (400 MHz, DMSO-d6) δ 8.23-8.17 (m, 1H), 7.54-7.45 (m, 1H), 5.87-5.82 (m, 1H), 5.13-4.96 (m, 2H), 2.90-2.66 (m, 2H), 2.40-2.14 (m, 2H), 1.18 (s, 9H).

Step 4: To a solution of (S)—N-(1-(3-chloro-2-fluoropyridin-4-yl)pent-4-en-1-ylidene)-2-methylpropane-2-sulfinamide I-29d (1.6 g, 5.0 mmol) and CuI (96.2 mg, 0.5 mmol) in THF (20 ml) at −78° C. was added and MeLi (4.3 ml, 1.6 M in diethyl ether, 6.9 mmol) dropwise. The reaction was allowed to warm to RT and stirred for 16 h then poured into saturated NH₄Cl aqueous (20 ml) and extracted with EtOAc (2×20 ml). The combined organics were washed with brine, dried over Na₂SO₄ and concentrated in vacuo. The product was purified by chromatography on silica gel (20-50% EtOAc/petroleum ether) to give (S)—N-(2-(3-chloro-2-fluoropyridin-4-yl)hex-5-en-2-yl)-2-methylpropane-2-sulfinamide I-29e as a yellow oil. LCMS (Method 5) m/z 333.1 (M+H)⁺ (ES⁺), at 1.48 min.

Step 5: 2-(3-Chloro-2-fluoropyridin-4-yl)hex-5-en-2-amine I-29f was synthesized from (S)—N-(2-(3-chloro-2-fluoropyridin-4-yl)hex-5-en-2-yl)-2-methylpropane-2-sulfinamide I-29e using a procedure essentially the same as for I-71. LCMS (Method 5) m/z 229.0 (M+H)⁺ (ES⁺), at 0.59 min

Step 6: N-(2-(3-Chloro-2-fluoropyridin-4-yl)hex-5-en-2-yl)-4-methoxyaniline I-29g was synthesized from 2-(3-chloro-2-fluoropyridin-4-yl)hex-5-en-2-amine I-29f using a procedure essentially the same as for I-7m. LCMS (Method 5) m/z 334.7 (M+H)⁺ (ES⁺), at 2.12 min

Step 7: To a solution of N-(2-(3-chloro-2-fluoropyridin-4-yl)hex-5-en-2-yl)-4-methoxy aniline I-29g (20 mg, 0.06 mmol) in toluene (2 ml) was added NaO^{t}Bu (9 mg, 0.09 mmol) and Pd-172 (2 mg, 0.003 mmol) at RT. The reaction was heated at 150° C. for 2 h in the microwave. After completion the reaction mixture was poured into water and extracted with EtOAc (2×20 ml), the combined organics were washed with brine, dried over Na₂SO₄ and concentrated in vacuo. The residue was purified by prep-TLC (33% EtOAc/petroleum ether) to give (5R,8S)-1-fluoro-10-(4-methoxyphenyl)-5-methyl-6,7,8,9-tetrahydro-5H-5,8-epiminocyclohepta[c]pyridine I-29 h as a pink solid. LCMS (Method 5) m/z 299.1 (M+H)⁺ (ES⁺), at 2.21 min

Step 8: (5R,8S)-1-fluoro-5-methyl-6,7,8,9-tetrahydro-5H 5,8 piminocyclohepta[c]pyridine I-29 was synthesized from (5R,8S)-1-fluoro-10-(4-methoxyphenyl)-5-methyl-6,7,8,9-tetrahydro-5H-5,8-epiminocyclohepta[c]pyridine I-29 h using a procedure essentially the same as for I-6. LCMS (Method 5) m/z 193.1 (M+H)⁺ (ES⁺), at 0.32 min.

### Intermediate 30 (I-30)

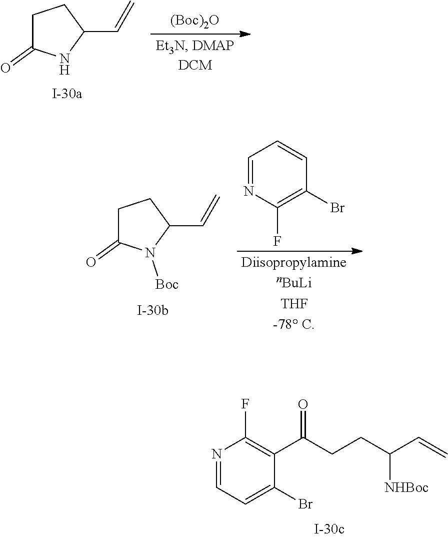

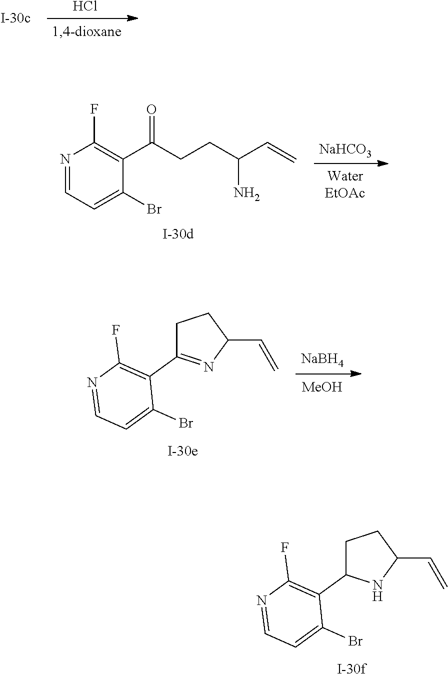

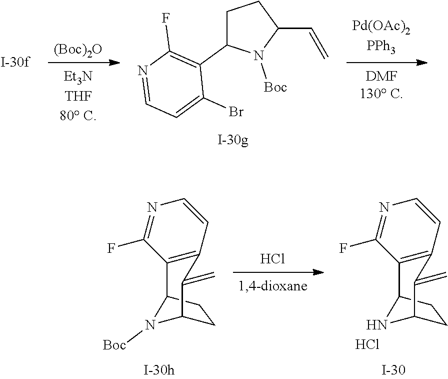

Step 1: To a solution of 5-vinylpyrrolidin-2-one I-30a (1.1 g, 10 mmol) in DCM (25 ml) was added di-tert-butyl dicarbonate (3.3 g, 15 mmol), DMAP (122 mg, 1 mmol) and Et₃N (4.12 ml, 30 mmol). The mixture was stirred at RT for 16 h. The mixture was concentrated in vacuo. The product was purified by chromatography on silica gel (5% EtOAc/petroleum ether) to give tert-butyl 2-oxo-5-vinylpyrrolidine-1-carboxylate I-30b as a yellow oil. ¹H NMR (400 MHz, DMSO-d6) δ 5.96 (m, 1H), 5.14-5.07 (m, 2H), 4.57-5.54 (m, 1H), 2.52-2.43 (m, 2H), 2.34-2.13 (m, 2H), 1.72-1.41 (m, 1H), 1.41 (s, 9H).

Step 2: To a solution of diisopropylamine (12 ml, 85.2 mmol) in THF (300 ml) at −78° C. under N₂ atmosphere was added a solution of ⁿBuLi in hexanes (35 ml, 2.5 M, 85.2 mmol). The mixture was stirred at −78° C. for 1 h then a solution of 3-bromo-2-fluoropyridine (12 g, 68.2 mmol) in THF (50 ml) was added. The reaction was stirred a further 1 h at −78° C. then a solution of tert-butyl 2-oxo-5-vinylpyrrolidine-1-carboxylate I-30b (14.4 g, 68.2 mmol) in THF (50 ml) was added. The reaction was stirred at −78° C. for 1 h then poured into a cooled (0° C.) solution of 1 M HCl (50 ml). The product was extracted with EtOAc (2×50 ml) and the combined organics were dried over Na₂SO₄ and concentrated in vacuo. The product was purified by chromatography on silica gel (10% EtOAc/petroleum ether) to give tert-butyl (6-(4-bromo-2-fluoropyridin-3-yl)-6-oxohex-1-en-3-yl)carbamate I-30c as a yellow solid. ¹H NMR (400 MHz, DMSO-d6) δ 8.23 (d, J=5.2 Hz, 1H), 7.79 (d, J=5.6 Hz, 1H), 6.94 (br. s, 1H), 5.78-5.70 (m, 1H), 5.10-5.02 (m, 2H), 3.98 (br. s, 1H), 2.95-2.84 (m, 2H), 1.84-1.71 (m, 2H), 1.37 (s, 9H)

Step 3: A solution of (6-(4-bromo-2-fluoropyridin-3-yl)-6-oxohex-1-en-3-yl)carbamate I-30c (8.6 g, 30 mmol) in a solution of HCl in 1,4-dioxane (30 ml, 40 mmol) was stirred at RT for 2 h. The mixture was concentrated in vacuo to give 4-amino-1-(4-bromo-2-fluoropyridin-3-yl)hex-5-en-1-one I-30d as a yellow oil. LCMS (Method 5) m/z 287.1, 289.1 (M+H)⁺ (ES⁺), at 1.28 min

Step 4: To a solution 4-amino-1-(4-bromo-2-fluoropyridin-3-yl)hex-5-en-1-one I-30d (8.9 g, 31 mmol) in a mixture of water (50 ml) and EtOAc (400 ml) was added NaHCO₃ (6.5 g, 77.5 mmol) and the mixture was stirred at RT for 16 h. The mixture was concentrated in vacuo and the product was purified by chromatography on silica gel column (20% EtOAc/petroleum ether) to give 4-bromo-2-fluoro-3-(2-vinyl-3,4-dihydro-2H-pyrrol-5-yl)pyridine I-30e as a yellow oil. LCMS (Method 5) m/z 269.0, 271.0 (M+H)⁺ (ES⁺), at 1.25 min

Step 5: To a solution of 4-bromo-2-fluoro-3-(2-vinyl-3,4-dihydro-2H-pyrrol-5-yl)pyridine I-30e (269 mg, 1 mmol) in a mixture of AcOH (0.85 ml) and MeOH (3 ml) at −40° C. was added NaBH₄ (87 mg, 2.3 mmol). The mixture was stirred at −40° C. for 4 h then the mixture was concentrated in vacuo. The residue obtained was partitioned between water and EtOAc and the aqueous further extracted with EtOAc (2×20 ml). The combined organics were washed with brine, dried over Na₂SO₄ and concentrated in vacuo. The product was purified by chromatography on silica gel (10% EtOAc/petroleum ether) to give 4-bromo-2-fluoro-3-(5-vinylpyrrolidin-2-yl)pyridine I-30f as a yellow oil. LCMS (Method 5) m/z 269.0, 271.0 (M+H)⁺ (ES⁺), at 0.93 min

Step 6: To a solution of 4-bromo-2-fluoro-3-(5-vinylpyrrolidin-2-yl)pyridine I-30f (1 g, 3.7 mmol) in THF (20 ml) was added di-tert-butyl dicarbonate (1.2 g, 5.5 mmol), and Et₃N (1.0 ml, 7.4 mmol). The mixture was stirred at RT for 16 h. The mixture was concentrated in vacuo. The product was purified by chromatography on silica gel (5% EtOAc/petroleum ether) to give tert-butyl 2-(4-bromo-2-fluoropyridin-3-yl)-5-vinylpyrrolidine-1-carboxylate I-30g as a yellow oil. ¹H NMR (400 MHz, DMSO-d6) δ 8.03-7.97 (m, 1H), 7.69-7.64 (m, 1H), 5.92-5.81 (m, 1H), 5.24-5.17 (m, 1H), 5.11-5.05 (m, 2H), 4.47-4.34 (m, 1H), 2.44-2.32 (m, 1H), 2.24-2.12 (m, 1H), 1.84-1.69 (m, 2H), 1.31-1.04 (s, 9H).

Step 7: To a solution of tert-butyl 2-(4-bromo-2-fluoropyridin-3-yl)-5-vinylpyrrolidine-1-carboxylate I-30g (500 mg, 1.35 mmol) in DMF (20 ml) was added Pd(OAc)₂ (30 mg, 0.135 mmol), PPh₃ (70.8 mg, 0.27 mmol) and Et₃N (375 μl, 2.7 mmol). The mixture was heated at 130° C. for 16 h under a N₂ atmosphere. The reaction mixture was cooled and diluted with water (30 ml). The product was extracted with EtOAc (2×20 ml). The combined organics were washed with brine (20 ml), dried over Na₂SO₄ and concentrated in vacuo. The product was purified by chromatography on silica gel (33% EtOAc/petroleum ether) to give tert-butyl (±)-1-fluoro-5-methylene-6,7,8,9-tetrahydro-5H-6,9-epiminocyclohepta[c]pyridine-10-carboxylate I-30 h as a yellow oil. ¹H NMR (400 MHz, CDCl₃) δ 8.03-7.97 (m, 1H), 7.69-7.64 (m, 1H), 5.92-5.81 (m, 1H), 5.24-5.17 (m, 1H), 5.11-5.05 (m, 2H), 4.47-4.34 (m, 1H), 2.44-2.32 (m, 1H), 2.24-2.12 (m, 1H), 1.84-1.69 (m, 2H), 1.31-1.04 (m, 9H)

Step 8: A solution of tert-butyl (±)-1-fluoro-5-methylene-6,7,8,9-tetrahydro-5H-6,9-epiminocyclohepta[c]pyridine-10-carboxylate I-30 h (50 mg, 0.17 mmol) in a solution of HCl in 1,4-dioxane (0.22 ml, 4 M 0.85 mmol) was stirred at RT for 2 h. The mixture was concentrated in vacuo to give (6S,9R)-1-fluoro-5-methylene-6,7,8,9-tetrahydro-5H-6,9-epiminocyclohepta[c]pyridine I-30 (50 mg, 100% yield) as a yellow oil. LCMS (Method 5) m/z 191.1 (M+H)⁺ (ES⁺), at 1.12 min

### Intermediate 31 (I-31)

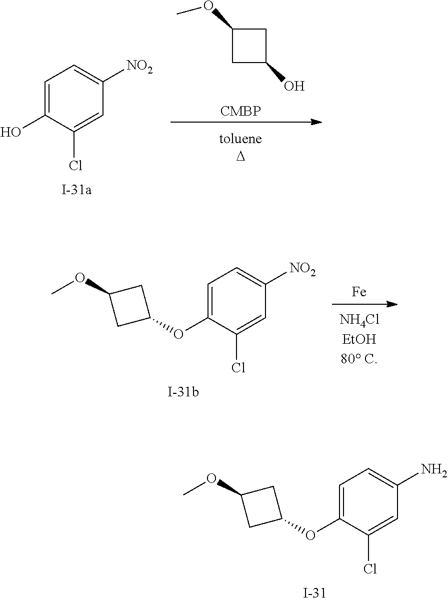

Step 1: To a solution of 2-chloro-4-nitrophenol (85 mg, 0.49 mmol) and (1s,3s)-3-methoxycyclobutan-1-ol (50 mg, 0.49 mmol) in toluene (2 ml) was added CMBP (236 mg, 0.98 mol). The reaction was heated at 120° C. for 16 h. The mixture was then diluted with water (20 ml) and the product was extracted with EtOAc (3×5 ml). The combined organics were washed with brine, dried over Na₂SO₄ and concentrated in vacuo. The product was purified by prep-TLC (20% EtOAc/petroleum ether) to give 2-chloro-1-((1r,3r)-3-methoxycyclobutoxy)-4-nitrobenzene I-31b as a white solid. ¹H NMR (400 MHz, DMSO-d6) δ 8.30 (d, J=2.8 Hz, 1H), 8.17 (dd, J=9.1, 2.8 Hz, 1H), 7.15 (d, J=9.2 Hz, 1H), 5.10-5.04 (m, 1H), 4.11-4.06 (m, 1H), 3.18 (s, 3H), 2.56-2.46 (m, 2H), 2.39-2.33 (m, 2H).

Step 2: To a solution of 2-chloro-1-((1r,3r)-3-methoxycyclobutoxy)-4-nitrobenzenol-31b (77 mg, 0.3 mol) in a mixture of EtOH (2.5 ml) and saturated aqueous NH₄Cl (2.5 ml) was added iron powder (135 mg, 2.4 mol). The reaction was heated at 80° C. for 16 h. The reaction was then allowed to cool to RT and filtered. The filtrate was extracted with EtOAc (2×10 ml) and the combined organics were washed with brine, dried over Na₂SO₄ and concentrated in vacuo to give 3-chloro-4-((1r,3r)-3-methoxycyclobutoxy)aniline I-31 as a brown oil. LCMS (Method 5) m/z 227.7 (M+H)⁺ (ES⁺), at 0.68 min

### Intermediate 62 (I-62)

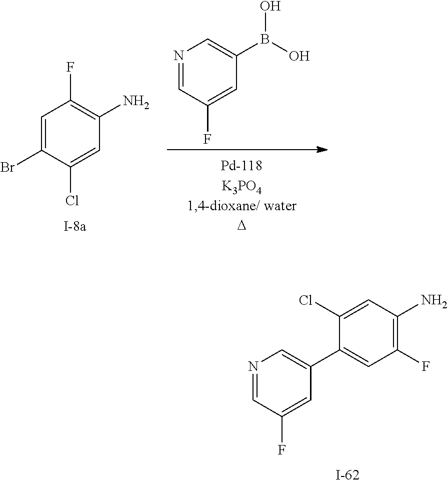

5-chloro-2-fluoro-4-(5-fluoropyridin-3-yl)aniline I-62 was synthesised from 4-bromo-5-chloro-2-fluoroaniline I-8a and (5-fluoropyridin-3-yl)boronic acid using a procedure essentially the same as for I-32. ¹H NMR (400 MHz, DMSO-d6) δ 8.55 (d, J=2.8 Hz, 1H), 8.48 (t, J=1.8 Hz, 1H), 7.80 (ddd, J=10.1, 2.8, 1.8 Hz, 1H), 7.24 (d, J=11.9 Hz, 1H), 6.93 (d, J=8.3 Hz, 1H), 5.75 (s, 2H).

### Intermediate 72: (I-72)

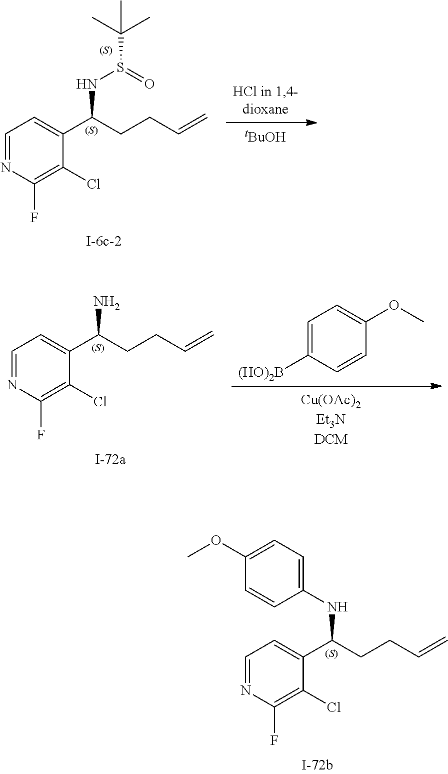

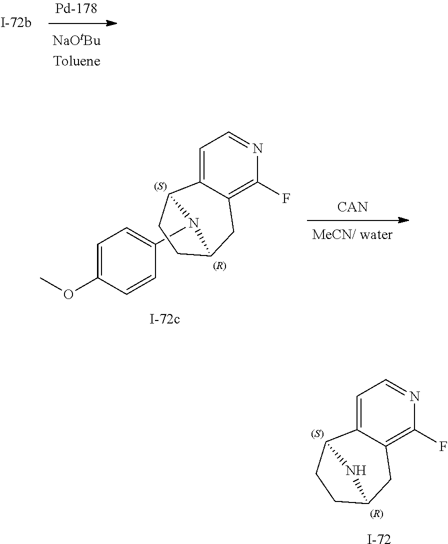

Step 1: (S)-1-(3-Chloro-2-fluoropyridin-4-yl)pent-4-en-1-amine I-72a was synthesised from (S)—N—((S)-1-(3-chloro-2-fluoropyridin-4-yl)pent-4-en-1-yl)-2-methylpropane-2-sulfinamide I-6c-2 using a procedure essentially the same as for I-6d. ¹H NMR (400 MHz, DMSO-d6) δ 8.15 (d, J=4.4 Hz, 1H), 7.62 (d, J=5.2 Hz, 1H), 5.85-5.75 (m, 1H), 5.04-4.93 (m, 2H), 4.21-4.18 (m, 1H), 2.14-2.10 (m, 4H), 2.14-1.56 (m, 2H).

Step 2: To a solution of (S)-1-(3-chloro-2-fluoropyridin-4-yl)pent-4-en-1-amine I-72a (6.7 g, 31.3 mmol) and (4-methoxyphenyl)boronic acid (19 g, 125.2 mmol) in DCM (120 ml) was added Cu(OAc)₂ (11.4 g, 62.6 mmol) and Et₃N (21.7 ml, 156.5 mmol). The reaction was stirred at RT for 72 h under a balloon of O₂. Aqueous NaOH solution (2 M, 100 ml) was added, the resultant mixture was filtered through a pad of celite and washed with DCM. The layers were separated, and the aqueous layer was extracted with DCM (2×300 ml). The combined organics were dried over Na₂SO₄, filtered and concentrated in vacuo. The product was purified by chromatography on silica gel (6% EtOAc/petroleum ether) to give (S)—N-(1-(3-chloro-2-fluoropyridin-4-yl)pent-4-en-1-yl)-4-methoxyaniline I-72b as a red oil. 1H NMR (400 MHz, DMSO-d6) δ 8.10 (d, J=5.2 Hz, 1H), 7.41 (d, J=5.2 Hz, 1H), 6.64 (d, J=9.2 Hz, 2H), 6.38 (d, J=8.8 Hz, 2H), 6.08 (d, J=8.4 Hz, 1H), 5.87-5.80 (m, 1H), 5.05-4.97 (m, 2H), 4.70-4.65 (m, 1H), 3.57 (s, 3H), 2.23-2.17 (m, 2H), 1.79-1.75 (m, 2H).

Step 3: A flask was loaded with Pd-172 (805 mg, 1.32 mmol) and NaO^{t}Bu (3.82 g, 39.88 mmol) and the flask was purged with N₂. A solution of (S)—N-(1-(3-chloro-2-fluoropyridin-4-yl)pent-4-en-1-yl)-4-methoxyaniline I-72b (8.51 g, 26.59 mmol) in toluene (150 ml) was added at RT. The resulting mixture was heated at 95° C. for 90 min. The mixture was cooled to RT and filtered through a path of Celite. The filter cake was washed with EtOAc (3×20 ml). The filtrate was concentrated in vacuo and the product was purified by chromatography on silica gel (6% EtOAc/petroleum ether) to afford (5S,8R)-1-fluoro-10-(4-methoxyphenyl)-6,7,8,9-tetrahydro-5H-5,8-epiminocyclohepta[c]pyridine I-72c as a white solid. ¹H NMR (400 MHz, DMSO-d6) δ 7.95 (d, J=4.8 Hz, 1H), 7.29 (dd, J=5.2, 2.0 Hz, 1H), 6.81-6.78 (m, 2H), 6.71-6.69 (m, 2H), 4.91 (d, J=4.8 Hz, 1H), 4.55 (t, J=5.6 Hz, 1H), 3.61 (s, 3H), 2.92 (dd, J=17.6, 4.8 Hz, 1H), 2.35 (d, J=17.6 Hz, 1H), 2.28-2.26 (m, 2H), 1.87-1.75 (m, 2H).

Step 4: (5S,8R)-1-fluoro-6,7,8,9-tetrahydro-5H-5,8-epiminocyclohepta[c]pyridine I-72 was synthesised from (5S,8R)-1-fluoro-10-(4-methoxyphenyl)-6,7,8,9-tetrahydro-5H-5,8-epiminocyclohepta[c]pyridine I-72c using a procedure that is essentially the same as for I-6. ¹H NMR (400 MHz, DMSO-d6) δ 7.91 (d, J=4.8 Hz, 1H), 7.04 (dd, J=8.8, 2.0 Hz, 1H), 4.17 (d, J=5.2 Hz, 1H), 3.78 (t, J=6.4 Hz, 1H), 2.88-2.76 (m, 2H), 2.38 (d, J=17.2 Hz, 1H), 1.98-1.88 (m, 2H), 1.75-1.70 (m, 1H), 1.56-1.45 (m, 1H).

### Intermediate 78 (I-78)

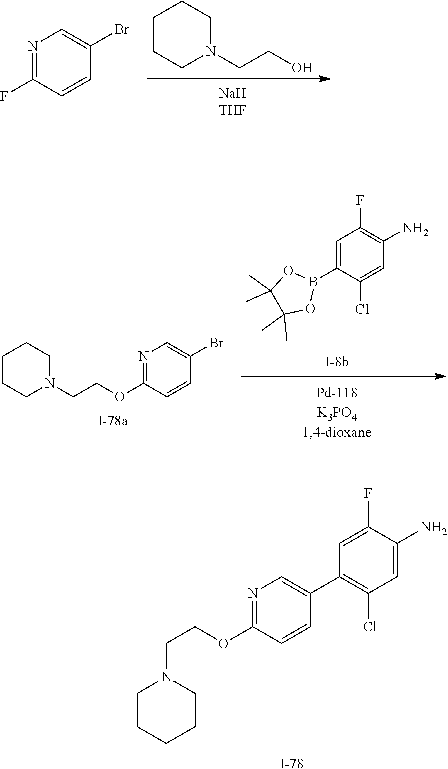

Step 1: To a mixture of sodium hydride (145 mg, 60% w/w, 3.64 mmol) in THF (10 ml) at 0° C. was added 2-(piperidin-1-yl)ethan-1-ol (453 μl, 3.41 mmol) and the resulting suspension stirred at 0° C. for 30 min. 5-bromo-2-fluoropyridine (234 μl, 2.27 mmol) was added and the reaction mixture was allowed to warm to RT for 6 h. Water (10 ml) and a 1:1 mixture of aqueous NaCl:NaHCO₃ (25 ml) was added. The product was extracted with DCM (2×25 ml). The combined organics were concentrated in vacuo. The product was purified by chromatography on silica gel (0-50% EtOAc/isohexane) to afford 5-bromo-2-(2-(piperidin-1-yl)ethoxy)pyridine I-78a as a colourless oil. ¹H NMR (400 MHz, DMSO-d6) δ 8.26 (dd, J=2.7, 0.6 Hz, 1H), 7.88 (dd, J=8.8, 2.6 Hz, 1H), 6.82 (dd, J=8.8, 0.7 Hz, 1H), 4.31 (t, J=6.0 Hz, 2H), 2.61 (t, J=6.0 Hz, 2H), 2.39 (t, J=5.3 Hz, 4H), 1.47 (p, J=5.5 Hz, 4H), 1.36 (q, J=6.1 Hz, 2H).

Step 2: 5-chloro-2-fluoro-4-(6-(2-(piperidin-1-yl)ethoxy)pyridin-3-yl)aniline I-78 was synthesised from 5-bromo-2-(2-(piperidin-1-yl)ethoxy)pyridine I-78a and 5-chloro-2-fluoro-4-(4,4,5,5-tetramethyl-1,3,2-dioxaborolan-2-yl)aniline I-8b using a procedure essentially the same as for I-47. ¹H NMR (400 MHz, DMSO-d6) δ 8.12 (d, J=2.5 Hz, 1H), 7.71 (dd, J=8.6, 2.5 Hz, 1H), 7.11 (d, J=11.9 Hz, 1H), 6.90 (d, J=8.3 Hz, 1H), 6.84 (d, J=8.5 Hz, 1H), 5.59 (s, 2H), 4.36 (t, J=6.0 Hz, 2H), 2.64 (t, J=6.0 Hz, 2H), 2.41 (t, J=5.4 Hz, 4H), 1.49 (p, J=5.5 Hz, 4H), 1.37 (td, J=6.4, 3.3 Hz, 2H).

### Intermediate 86 (I-86)

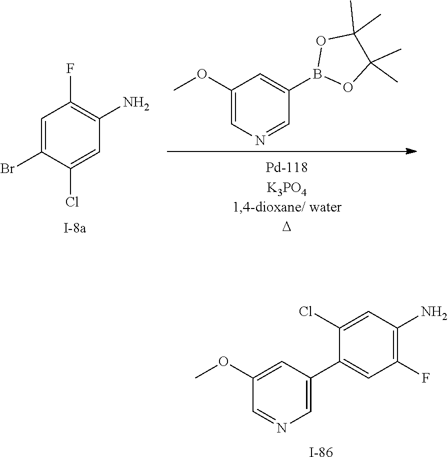

5-chloro-2-fluoro-4-(5-methoxypyridin-3-yl)aniline I-86 was synthesised from 4-bromo-5-chloro-2-fluoroaniline I-8a and 3-methoxy-5-(4,4,5,5-tetramethyl-1,3,2-dioxaborolan-2-yl)pyridine using a procedure essentially the same as for I-32. ¹H NMR (400 MHz, DMSO-d6) δ 8.25 (d, J=2.8 Hz, 1H), 8.18 (d, J=1.8 Hz, 1H), 7.37 (dd, J=2.8, 1.8 Hz, 1H), 7.19 (d, J=12.0 Hz, 1H), 6.92 (d, J=8.3 Hz, 1H), 5.67 (s, 2H), 3.86 (s, 3H).

### Intermediate 104 (I-104)

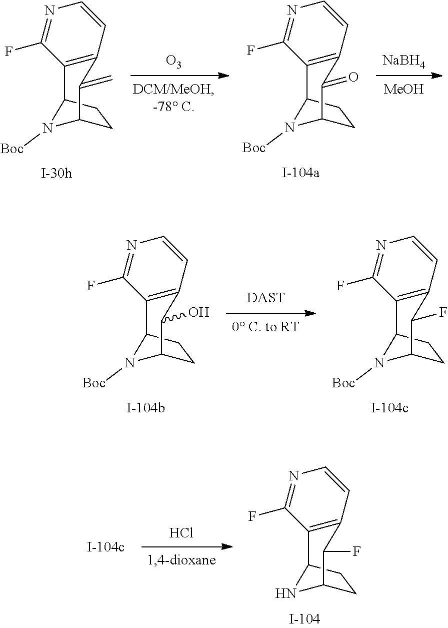

Step 1: To a solution of tert-butyl(±)-1-fluoro-5-methylene-6,7,8,9-tetrahydro-5H-6,9-epiminocyclohepta[c]pyridine-10-carboxylate I-30 h (200 mg, 0.68 mmol) in MeOH (5 ml) was bubbled O₃ at −78° C. for 10 min. After completion, a drop of dimethyl sulfide was added into the reaction mixture and the reaction solution turned colourless. The mixture was concentrated in vacuo. The product was purified by prep-TLC (33% EtOAc/petroleum ether) to give tert-butyl (±)-1-fluoro-5-oxo-6,7,8,9-tetrahydro-5H-6,9-epiminocyclohepta[c]pyridine-10-carboxylate I-104-a as a white solid. LCMS (Method 4) m/z 293.1 (M+H)⁺ (ES⁺), at 1.87 min

Step 2: To a solution tert-butyl (±)-1-fluoro-5-oxo-6,7,8,9-tetrahydro-5H-6,9-epiminocyclohepta[c]pyridine-10-carboxylate I-104a (90 mg, 0.33 mmol) in THF (5 ml) was added NaBH₄ (26 mg, 0.66 mol) under N₂ at RT. The reaction mixture was stirred at RT for 1 h. The reaction mixture was filtered and concentrated in vacuo. The product was purified by prep-TLC (50% EtOAc/petroleum ether) to give tert-butyl (±)-1-fluoro-5-hydroxy-6,7,8,9-tetrahydro-5H-6,9-epiminocyclohepta[c]pyridine-10-carboxylate I-104b as a colourless oil. LCMS (Method 5) m/z 295.1 (M+H)⁺ (ES⁺), at 1.51 min

Step 3: To a solution of tert-butyl (±)-1-fluoro-5-hydroxy-6,7,8,9-tetrahydro-5H-6,9-epiminocyclohepta[c]pyridine-10-carboxylate I-104b (30 mg, 0.1 mmol) in DCM (5 ml) was added DAST (44.2 mg, 0.2 mol) at 0° C. under N₂. The reaction mixture was allowed to warm to RT and stirred for 16 h. The reaction mixture was basified with saturated aqueous NaHCO₃ solution and extracted with DCM (3×10 ml). The product was purified by chromatography on silica gel (33% EtOAc/petroleum ether) to give tert-butyl (±, 5-endo)-1,5-difluoro-6,7,8,9-tetrahydro-5H-6,9-epiminocyclohepta[c]pyridine-10-carboxylate I-104c as a yellow oil. LCMS (Method 5) m/z 296.7 (M+H)⁺ (ES⁺), at 1.67 min

Step 4: To a solution tert-butyl (±, 6-endo)-1,5-difluoro-6,7,8,9-tetrahydro-5H-6,9-epiminocyclohepta[c]pyridine-10-carboxylate I-104c (15 mg, 0.05 mmol) in 1,4-dioxane (3 ml) was added a solution of HCl in 1,4-dioxane (4 M, 3 ml). The reaction mixture was stirred at RT for 1 h. The reaction mixture was basified with saturated aqueous NaHCO₃ solution and extracted with DCM (3×10 ml). The combined organics were dried over Na₂SO₄ and concentrated in vacuo to afford (±,5-endo)-1,5-difluoro-6,7,8,9-tetrahydro-5H-6,9-epiminocyclohepta[c]pyridine as a yellow oil.

### Intermediate 109 (I-109)

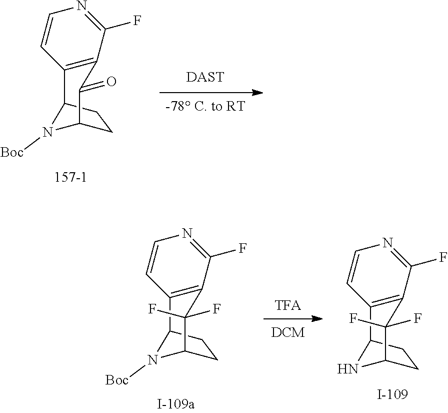

Step 1: To a solution of tert-butyl 1-fluoro-9-oxo-6,7,8,9-tetrahydro-5H-5,8-epiminocyclohepta[c]pyridine-10-carboxylate 157-1 (998 mg, 3.42 mmol) in DCM (16.5 ml) was added DAST (3.30 g, 20.50 mol) at −78° C. The reaction mixture was allowed to warm to RT and stirred for 16 h. The mixture was basified with saturated aqueous NaHCO₃ solution and extracted with DCM (3×30 ml). The combined organics were dried over Na₂SO₄ and concentrated in vacuo. The product was purified by chromatography on silica gel (33% EtOAc/petroleum ether) to give tert-butyl 1,9,9-trifluoro-6,7,8,9-tetrahydro-5H-5,8-epiminocyclo hepta[c]pyridine-10-carboxylate I-109a as a light yellow solid. ¹H NMR (400 MHz, DMSO-d6) δ 8.34 (d, J=5.2 Hz, 1H), 7.48 (dd, J=5.2, 1.6 Hz, 1H), 5.26 (d, J=6.8 Hz, 1H), 4.80-4.75 (m, 1H), 2.34-2.10 (m, 2H), 1.89-1.81 (m, 1H), 1.69-1.64 (m, 1H), 1.39 (s, 9H)

Step 2: To a solution of tert-butyl 1,9,9-trifluoro-6,7,8,9-tetrahydro-5H-5,8-epimino cyclohepta[c]pyridine-10-carboxylate I-109a (83 mg, 0.26 mmol) in DCM (3 ml) was added TFA (1 ml). The reaction mixture was stirred at RT for 1 h. After this time the reaction mixture was concentrated in vacuo to afford 1,9,9-trifluoro-6,7,8,9-tetrahydro-5H-5,8-epiminocyclohepta[c]pyridine as a yellow oil. LCMS (Method 5) m/z 215.1 (M+H)⁺ (ES⁺), at 0.38 min.

### Intermediate 112 (I-112)

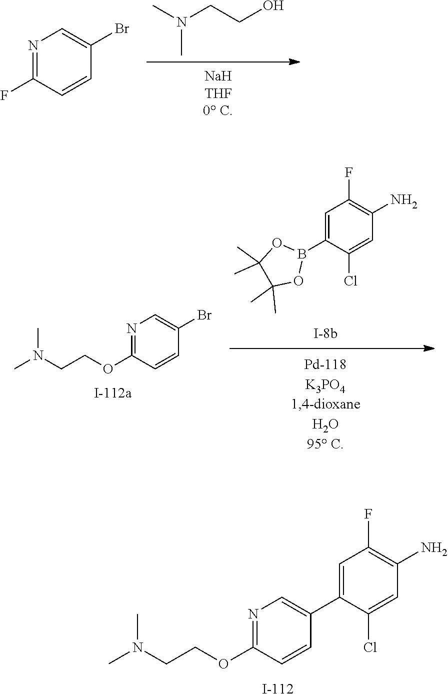

Step 1: 2-((5-Bromopyridin-2-yl)oxy)-N,N-dimethylethan-1-amine I-112a was synthesised from 2-(dimethylamino)ethan-1-ol and 5-bromo-2-fluoropyridine using a procedure essentially the same as for I-78a. ¹H NMR (400 MHz, DMSO-d6) δ 8.27 (dd, J=2.7, 0.7 Hz, 1H), 7.88 (dd, J=8.8, 2.6 Hz, 1H), 6.81 (dd, J=8.8, 0.7 Hz, 1H), 4.30 (t, J=5.8 Hz, 2H), 2.59 (t, J=5.9 Hz, 2H), 2.18 (s, 6H).

Step 2: 5-Chloro-4-(6-(2-(dimethylamino)ethoxy)pyridine-3-yl)-2-fluoroaniline I-112 was synthesised from 2-((5-bromopyridin-2-yl)oxy)-N,N-dimethylethan-1-amine I-112a and 5-chloro-2-fluoro-4-(4,4,5,5-tetramethyl-1,3,2-dioxaborolan-2-yl)aniline I-8b using a procedure essentially the same as for I-47. ¹H NMR (400 MHz, DMSO-d6) δ 8.13 (dd, J=2.6, 0.7 Hz, 1H), 7.72 (dd, J=8.6, 2.5 Hz, 1H), 7.12 (d, J=11.9 Hz, 1H), 6.90 (d, J=8.4 Hz, 1H), 6.83 (dd, J=8.6, 0.8 Hz, 1H), 5.59 (s, 2H), 4.35 (t, J=5.9 Hz, 2H), 2.62 (t, J=5.9 Hz, 2H), 2.21 (s, 6H).

### EXPERIMENTAL SCHEME 3

### Compound 60: (5R,8S)—N-(3-Chloro-4-(2-hydroxypropan-2-yl)phenyl)-1-fluoro-6,7,8,9-tetrahydro-5H-5,8-epiminocyclohepta[c]pyridine-10-carboxamide

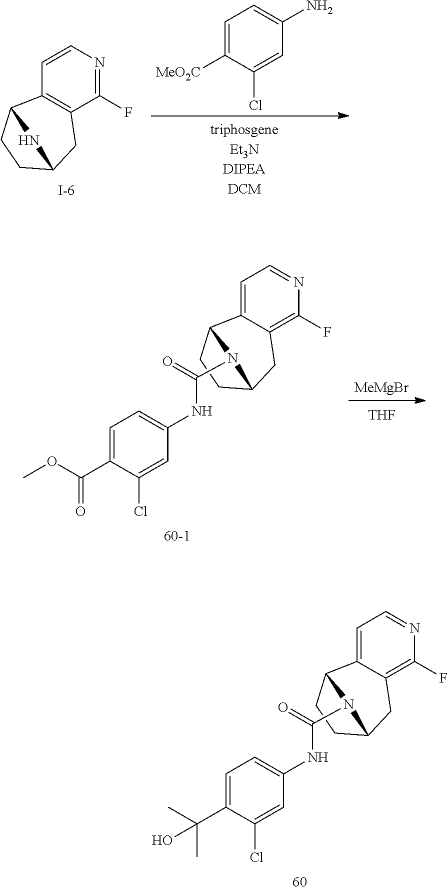

Step 1: To a solution of triphosgene (35 mg, 0.12 mmol) in DCM (1 ml) was added a solution of methyl 4-amino-2-chlorobenzoate (53 mg, 0.29 mmol) in DCM (1 ml), followed by slow addition of Et₃N (0.11 ml, 0.78 mmol) and the reaction mixture was stirred for 10 min. To a solution of (5R,8S)-1-fluoro-6,7,8,9-tetrahydro-5H-5,8-epiminocyclohepta[c]pyridine I-6 (50 mg, 0.26 mmol) in DCM (2 ml) was added the solution of isocyanate, followed by addition of DIPEA (0.13 ml, 0.78 mmol) and the reaction was stirred at RT for 18 h. The reaction was concentrated in vacuo. The product was purified by chromatography on silica gel (0-65% EtOAc/isohexane) to afford methyl 2-chloro-4-((5R,8S)-1-fluoro-6,7,8,9-tetrahydro-5H-5,8-epiminocyclohepta[c]pyridine-10-carboxamido)benzoate 60-1 as an off white solid. LCMS (Method 1) m/z 390.1, 392.1 (M+H)⁺ (ES⁺), at 1.26 min. ¹H NMR (500 MHz, DMSO-d6) δ 9.19 (s, 1H), 8.01 (d, J=5.0 Hz, 1H), 7.81-7.75 (m, 2H), 7.53 (dd, J=8.7, 2.1 Hz, 1H), 7.21 (dd, J=5.1, 1.6 Hz, 1H), 5.26 (d, J=6.1 Hz, 1H), 4.83 (t, J=6.4 Hz, 1H), 3.80 (s, 3H), 3.15 (dd, J=17.3, 5.0 Hz, 1H), 2.60 (d, J=17.3 Hz, 1H), 2.30-2.10 (m, 2H), 1.94-1.80 (m, 1H), 1.78-1.74 (m 1H)

Step 2: A solution of methyl 2-chloro-4-((5R,8S)-1-fluoro-6,7,8,9-tetrahydro-5H-5,8-epiminocyclohepta[c]pyridine-10-carboxamido)benzoate 60-1 (15 mg, 38 μmol) in THF (2 ml) was cooled to 0° C. in an ice bath. A solution of methylmagnesium bromide in diethyl ether (51 μl, 3 M, 0.15 mmol) was added dropwise and the reaction mixture was stirred at RT for 2 h. The reaction mixture was heated to 50° C. for 1 h. The reaction mixture was stirred at RT for 2 days. The reaction mixture was cooled to 0° C. and a further portion of methylmagnesium bromide solution in diethyl ether (51 μl, 3 M, 0.15 mmol) was added. A saturated NH₄Cl solution (10 ml) was added and the product was extracted with 10% MeOH in DCM solution (3×5 ml). The combined organics were dried and concentrated in vacuo. The product was purified by chromatography on silica gel (0-3% MeOH/DCM) to give (5R,8S)—N-(3-chloro-4-(2-hydroxypropan-2-yl)phenyl)-1-fluoro-6,7,8,9-tetrahydro-5H-5,8-epiminocyclohepta[c]pyridine-10-carboxamide 60 as a colourless gum. LCMS (Method 1) m/z 390.1, 392.1 (M+H)⁺ (ES⁺), at 1.34 min. ¹H NMR (500 MHz, DMSO-d6) δ 9.20 (s, 1H), 8.01 (d, J=5.1 Hz, 1H), 7.80-7.77 (m, 2H), 7.55-7.52 (m, 1H), 7.21 (d, J=5.7 Hz, 1H), 5.26 (d, J=6.1 Hz, 1H), 4.85-4.81 (m, 1H), 3.19-3.12 (m, 1H), 2.62 (s, 1H), 2.30-2.14 (m, 2H), 2.05-1.91 (m, 1H), 1.80-1.72 (m, 1H), 1.23 (s, 6H)

### Experimental Scheme 4

### Compound 87: (±)—N-(4,5-Dichloro-2-fluorophenyl)-4-fluoro-6,7,8,9-tetrahydro-5H-6,9-epiminocyclohepta[d]pyrimidine-10-carboxamide

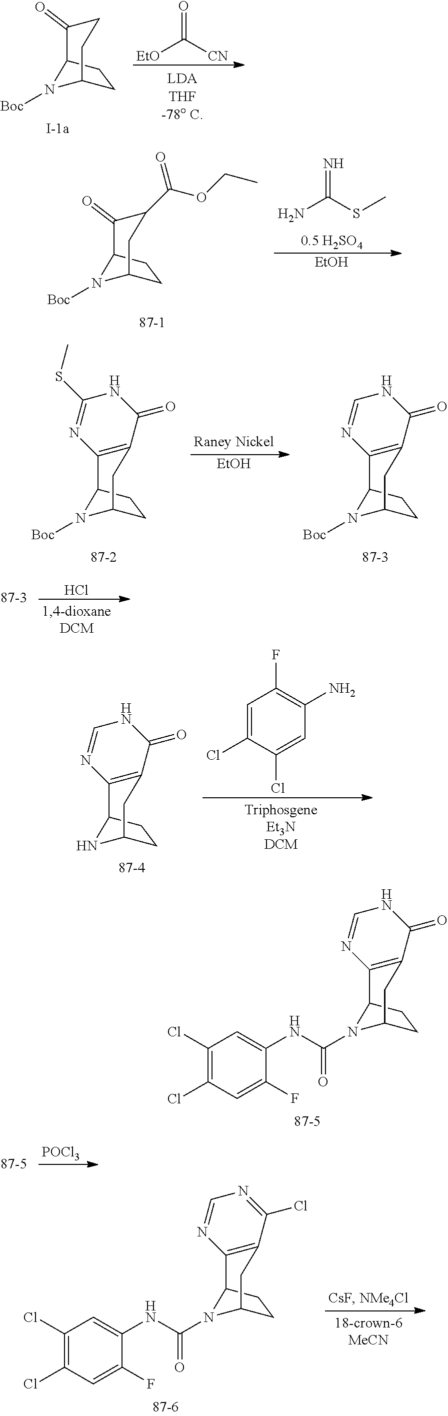

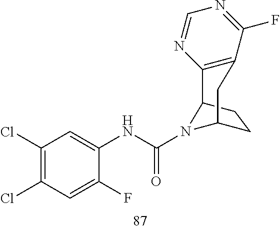 [SMILES: O=C(Nc1cc(Cl)c(Cl)cc1F)N1C2CCC1c1ncnc(F)c1C2]

Step 1: To a solution of tert-butyl (±)-2-oxo-8-azabicyclo[3.2.1]octane-8-carboxylate I-1a (2.26 g, 10.0 mmol) in THF (30 ml) at −78° C. was added a solution of lithium diisopropylamide in heptane/THF/ethylbenzene (6 ml, 2.0 M, 12.0 mmol) and the resulting solution was stirred at −78° C. for 1.5 h. Ethyl carbonocyanidate (1.49 ml, 15.0 mmol) was added dropwise at −78° C. and the mixture was left to slowly warm to RT over 16 h. EtOAc (75 ml), water (100 ml) and brine (50 ml) were added and the layers were separated. The aqueous layer was further extracted with EtOAc (2×75 ml) and the combined organics were dried over MgSO₄ and concentrated in vacuo. The product was purified by chromatography on silica gel (0-100% EtOAc/isohexane) to afford 8-(tert-butyl) 3-ethyl (±)-2-oxo-8-azabicyclo[3.2.1]octane-3,8-dicarboxylate 87-1 as a thick colourless oil. ¹H NMR (400 MHz, CDCl₃) δ 11.93 (s, 1H), 4.35 (br. s, 2H), 4.19 (q, J=7.1 Hz, 2H), 2.79 (br. s, 1H), 2.25-2.01 (m, 3H), 1.96 (d, J=15.6 Hz, 1H), 1.66-1.55 (m, 1H), 1.44 (s, 9H), 1.28 (t, J=7.1 Hz, 3H).

Step 2: To a solution of 8-(tert-butyl) 3-ethyl (±)-2-oxo-8-azabicyclo[3.2.1]octane-3,8-dicarboxylate 87-1 (1.00 g, 3.36 mmol) and S-methylisothiourea hemisulfate salt (4.68 g, 33.6 mmol) in EtOH (16 ml) was added NaHCO₃ (2.83 g, 33.6 mmol) and the resulting mixture was stirred at 50° C. for 16 h. DCM (100 ml) and sat. aq. NH₄Cl (100 ml) were added and the layers were separated. The aqueous layer was further extracted with DCM (100 ml) and DCM/MeOH (8:2, 50 ml). The combined organics were dried over MgSO₄ and concentrated in vacuo. The product was purified by chromatography on silica gel (0-100% EtOAc/isohexane) to afford tert-butyl(±)-4-hydroxy-2-(methylthio)-6,7,8,9-tetrahydro-5H-6,9-epiminocyclohepta[d]pyrimidine-10-carboxylate 87-2 as a colourless oil which became a white solid upon standing. ¹H NMR (400 MHz, CDCl₃) δ 4.72 (s, 1H), 4.49 (s, 1H), 3.07-2.87 (m, 1H), 2.58 (s, 3H), 2.36-2.11 (m, 3H), 2.03-1.93 (m, 1H), 1.70-1.56 (m, 1H), 1.44 (s, 9H). Exchangeable proton not observed.

Step 3: To a flask flushed with N₂ was added Raney nickel 2800, slurry in water (2.0 ml), followed by a solution of tert-butyl(±)-4-hydroxy-2-(methylthio)-6,7,8,9-tetrahydro-5H-6,9-epiminocyclohepta[d]pyrimidine-10-carboxylate 87-2 (370 mg, 1.09 mmol) in EtOH (20.0 ml). The resulting mixture was stirred at reflux for 3 h. Additional Raney nickel 2800, slurry in water (5.0 ml) was added and the mixture was stirred at reflux for 1.5 h. A further portion of Raney nickel 2800, slurry in water (5.0 mL) was added and the mixture was stirred at reflux for 30 min. The mixture was cooled and filtered through a glass fiber filter paper, washing with EtOH. The filtrate was concentrated in vacuo to afford tert-butyl (±)-4-hydroxy-6,7,8,9-tetrahydro-5H-6,9-epiminocyclohepta[d]pyrimidine-10-carboxylate 87-3 as a pale green foam. ¹H NMR (400 MHz, DMSO-d6) δ 8.00 (s, 1H), 4.45 (d, J=5.8 Hz, 1H), 4.35 (s, 1H), 2.74 (dd, J=17.5, 4.8 Hz, 1H), 2.22-1.96 (m, 3H), 1.83 (t, J=9.7 Hz, 1H), 1.62-1.49 (m, 1H), 1.35 (s, 9H). Exchangeable proton not observed.

Step 4: To a solution of tert-butyl (±)-4-hydroxy-6,7,8,9-tetrahydro-5H-6,9-epiminocyclohepta[d]pyrimidine-10-carboxylate 87-3 (70 mg, 0.25 mmol) in DCM (3 ml) was added a solution of HCl in 1,4-dioxane (2 ml, 4.0 M, 8.0 mmol) and the resulting mixture was stirred at RT for 1 h. The solvent was removed in vacuo and the residue was triturated with MeOH (3×5 ml) to afford (±)-6,7,8,9-tetrahydro-5H-6,9-epiminocyclohepta[d]pyrimidin-4-ol, HCl salt 87-4 as a yellow oil. The material was used in the next step without further analysis or purification

Step 5: To a solution of 4,5-dichloro-2-fluoroaniline (46 mg, 0.25 mmol) in DCM (1 ml) was added Et₃N (0.19 ml, 1.4 mmol) followed by a solution of triphosgene (27 mg, 93 μmol) in DCM (1 ml). The mixture was stirred for 15 min, before the addition of a solution of (±)-6,7,8,9-tetrahydro-5H-6,9-epiminocyclohepta[d]pyrimidin-4-ol, HCl salt 87-4 (41 mg, 0.23 mmol) in DCM (2 ml). The resulting mixture was stirred at RT for 16 h. The reaction mixture was concentrated in vacuo and the resulting residue was purified by chromatography on RP Flash C₁₈ (15-60% (0.1% Formic acid in MeCN)/(0.1% formic acid in water)) to afford (±)—N-(4,5-dichloro-2-fluorophenyl)-4-hydroxy-6,7,8,9-tetrahydro-5H-6,9-epiminocyclohepta[d]pyrimidine-10-carboxamide 87-5 as a pink solid. ¹H NMR (400 MHz, DMSO-d6) δ 8.81 (s, 1H), 8.03 (s, 1H), 7.87-7.79 (m, 1H), 7.71-7.61 (m, 1H), 4.79 (d, J=6.1 Hz, 1H), 4.71-4.63 (m, 1H), 2.88 (dd, J=17.8, 4.9 Hz, 1H), 2.27-2.01 (m, 3H), 1.95-1.86 (m, 1H), 1.70-1.59 (m, 1H). One exchangeable proton not observed.

Step 6: To a vial containing (±)—N-(4,5-dichloro-2-fluorophenyl)-4-hydroxy-6,7,8,9-tetrahydro-5H-6,9-epiminocyclohepta[d]pyrimidine-10-carboxamide 87-5 (30 mg, 78 μmol) was added POCl₃ (1.1 ml, 12 mmol) and the resulting mixture was stirred at 100° C. for 1 h. The mixture was cooled and diluted with toluene (3 ml) and the solvent was removed under reduced pressure. The residue was taken up in toluene (3 ml) and evaporated two more times. The material was taken up in DCM/MeOH and concentrated onto silica. The product was purified by chromatography on silica gel (0-100% EtOAc/isohexane) to afford (±)-4-chloro-N-(4,5-dichloro-2-fluorophenyl)-6,7,8,9-tetrahydro-5H-6,9-epiminocyclohepta[d]pyrimidine-10-carboxamide 87-6 as a white solid. ¹H NMR (400 MHz, CDCl₃) δ 8.79 (s, 1H), 7.15 (d, J=9.5 Hz, 1H), 6.95 (d, J=7.7 Hz, 1H), 5.33-5.22 (m, 1H), 4.99-4.84 (m, 1H), 3.52 (d, J=17.7 Hz, 1H), 2.64 (d, J=17.7 Hz, 1H), 2.58-2.40 (m, 2H), 2.16-2.05 (m, 1H), 1.90-1.72 (m, 1H). Exchangeable NH not observed.

Step 7: To a vial charged with (±)-4-chloro-N-(4,5-dichloro-2-fluorophenyl)-6,7,8,9-tetrahydro-5H-6,9-epiminocyclohepta[d]pyrimidine-10-carboxamide 87-6 (14 mg, 35 μmol) under N₂ was added 18-crown-6 (0.46 mg, 1.7 μmol) and tetramethyl NH₄Cl (0.16 μl, 1.7 μmol). MeCN (0.5 ml) was added and the mixture was stirred and sonicated until the mixture became homogeneous. Cesium fluoride (30 mg, 0.20 mmol) was added and the mixture was heated at 60° C. for 16 h. Additional cesium fluoride (30 mg, 0.20 mmol) was added and the mixture was stirred at 60° C. for 72 h. The mixture was filtered over celite, rinsing thoroughly with MeCN (10 ml). The filtrate was concentrated in vacuo. The product was purified by chromatography on silica gel (0-10% (0.7 M NH₃/MeOH)/DCM). to afford (±)—N-(4,5-dichloro-2-fluorophenyl)-4-fluoro-6,7,8,9-tetrahydro-5H-6,9-epiminocyclohepta[d]pyrimidine-10-carboxamide as a pink solid. LCMS (Method 1) m/z 384.2, 386.3 (M+H)⁺ (ES⁺), at 1.42 min. ¹H NMR (500 MHz, DMSO-d6) δ 8.95 (s, 1H), 8.72 (s, 1H), 7.81 (d, J=7.5 Hz, 1H), 7.66 (d, J=10.3 Hz, 1H), 5.14 (d, J=5.9 Hz, 1H), 4.80 (t, J=6.1 Hz, 1H), 3.23 (dd, J=17.5, 4.9 Hz, 1H), 2.62 (d, J=17.2 Hz, 1H), 2.32-2.18 (m, 2H), 1.95-1.87 (m, 1H), 1.82-1.73 (m, 1H).

### Experimental Scheme 5

### Compound 99 and 100: 99 (6S,9R)-4-Chloro-N-(4,5-dichloro-2-fluorophenyl)-1-fluoro-6,7,8,9-tetrahydro-5H-6,9-epiminocyclohepta[c]pyridine-10-carboxamide

### 100 (5R,8S)-4-Chloro-N-(4,5-dichloro-2-fluorophenyl)-1-fluoro-6,7,8,9-tetrahydro-5H-5,8-epiminocyclohepta[c]pyridine-10-carboxamide

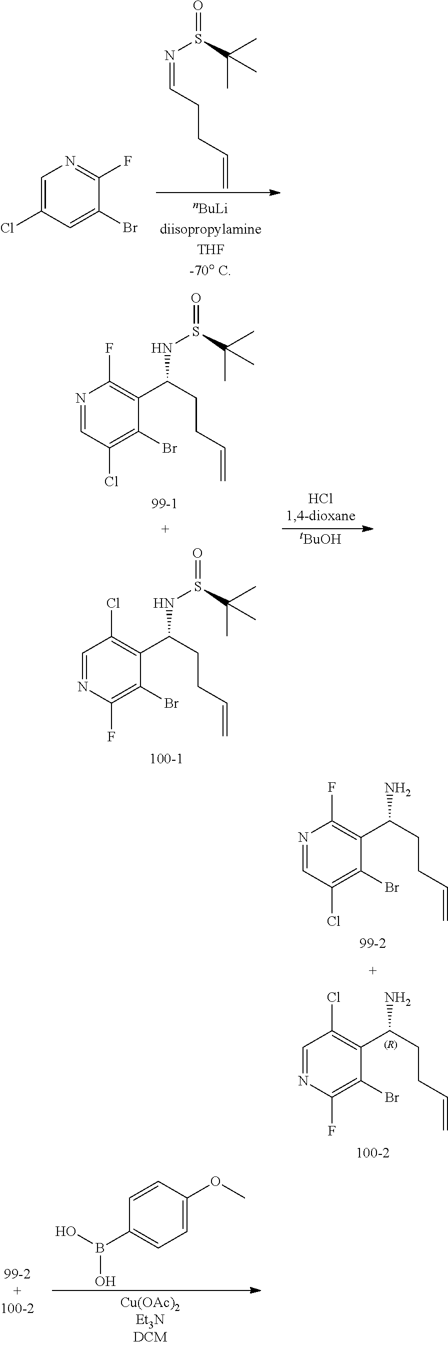

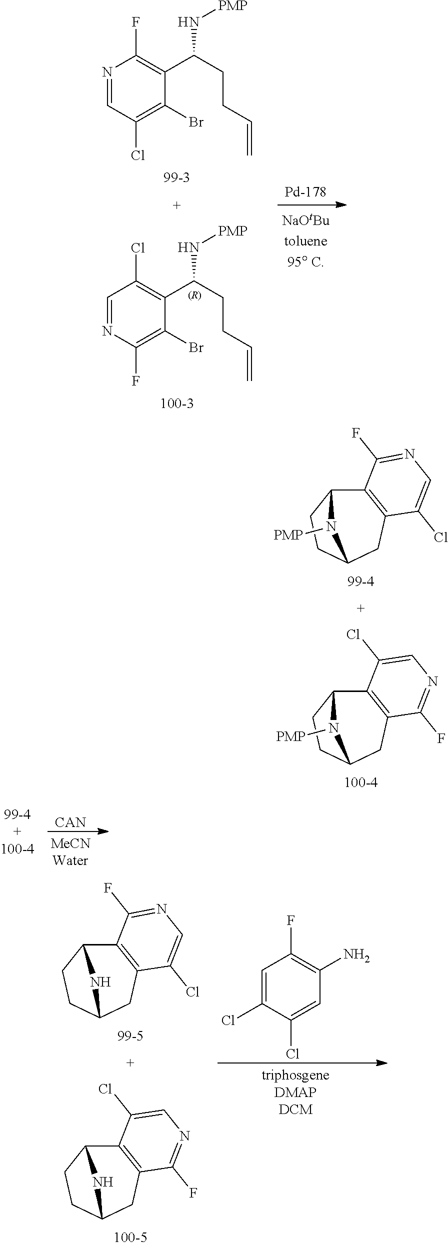

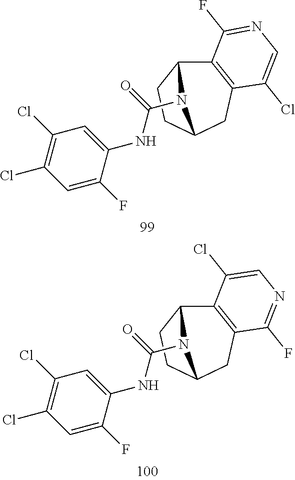 [SMILES: O=C(Nc1cc(Cl)c(Cl)cc1F)N1[C@H]2CC[C@@H]1c1c(F)ncc(Cl)c1C2 ; O=C(Nc1cc(Cl)c(Cl)cc1F)N1[C@H]2CC[C@@H]1c1c(Cl)cnc(F)c1C2]

Step 1: (S)—N—((R)-1-(4-Bromo-5-chloro-2-fluoropyridin-3-yl)pent-4-en-1-yl)-2-methylpropane-2-sulfinamide 99-1 and (S)—N—((R)-1-(3-bromo-5-chloro-2-fluoropyridin-4-yl)pent-4-en-1-yl)-2-methylpropane-2-sulfinamide 100-1 were synthesised from 3-bromo-5-chloro-2-fluoropyridine using a procedure essentially the same as I-21c. ¹H NMR (400 MHz, DMSO-d6) δ 8.42-8.27 (m, 1H), 5.83-8.72 (m, 1H), 5.13-4.96 (m, 2H), 2.26-2.21 (m, 2H), 2.20-1.89 (m, 2H), 1.04-1.01 (m, 9H).

Step 2: (R)-1-(4-Bromo-5-chloro-2-fluoropyridin-3-yl)pent-4-en-1-amine 99-2 and (R)-1-(3-Bromo-5-chloro-2-fluoropyridin-4-yl)pent-4-en-1-amine 100-2 were synthesised from (S)—N—((R)-1-(4-bromo-5-chloro-2-fluoropyridin-3-yl)pent-4-en-1-yl)-2-methylpropane-2-sulfinamide 99-1 and (S)—N—((R)-1-(3-bromo-5-chloro-2-fluoropyridin-4-yl)pent-4-en-1-yl)-2-methylpropane-2-sulfinamide 100-1 using a procedure essentially the same as for I-71. ¹H NMR (400 MHz, DMSO-d6) δ 8.33-8.27 (m, 1H), 5.85-5.75 (m, 1H), 5.04-4.93 (m, 2H), 4.53-4.28 (m, 1H), 2.33-2.11 (m, 3H), 2.03-1.73 (m, 2H).

Step 3: (R)—N-(1-(4-Bromo-5-chloro-2-fluoropyridin-3-yl)pent-4-en-1-yl)-4-methoxyaniline 99-3 and (R)—N-(1-(3-bromo-5-chloro-2-fluoropyridin-4-yl)pent-4-en-1-yl)-4-methoxyaniline 100-3 were synthesised from (R)-1-(4-bromo-5-chloro-2-fluoropyridin-3-yl)pent-4-en-1-amine 99-2 and (R)-1-(3-bromo-5-chloro-2-fluoropyridin-4-yl)pent-4-en-1-amine 100-2 using a procedure essentially the same as I-7m. ¹H NMR (400 MHz, DMSO-d6) δ 8.31 (s, 1H), 8.66-8.63 (m, 2H), 6.43-6.41 (m, 2H), 5.89-5.75 (m, 1H), 5.08-4.97 (m, 2H), 3.58 (s, 3H), 2.37-2.04 (m, 4H).

Step 4: (6S,9R)-4-Chloro-1-fluoro-10-(4-methoxyphenyl)-6,7,8,9-tetrahydro-5H-6,9-epiminocyclohepta[c]pyridine 99-4 and (5R,8S)-4-Chloro-1-fluoro-10-(4-methoxyphenyl)-6,7,8,9-tetrahydro-5H-5,8-epiminocyclohepta[c]pyridine 100-4 were synthesised from (R)—N-(1-(4-Bromo-5-chloro-2-fluoropyridin-3-yl)pent-4-en-1-yl)-4-methoxyaniline 99-3 and (R)—N-(1-(3-bromo-5-chloro-2-fluoropyridin-4-yl)pent-4-en-1-yl)-4-methoxyaniline 100-3 using a procedure essentially the same as for I-20 h. ¹H NMR (400 MHz, DMSO-d6) δ 8.12-8.08 (m, 1H), 8.80-8.69 (m, 4H), 5.03-4.97 (m, 1H), 4.61-4.59 (m, 1H), 3.61 (s, 3H), 2.97-2.87 (m, 1H), 2.46-2.37 (m, 1H), 2.34-2.23 (m, 2H), 1.96-1.77 (m, 2H).

Step 5: (6S,9R)-4-Chloro-1-fluoro-6,7,8,9-tetrahydro-5H-6,9-epiminocyclohepta[c]pyridine 99-5 and (5R,8S)-4-chloro-1-fluoro-6,7,8,9-tetrahydro-5H-5,8-epiminocyclohepta[c]pyridine 100-5 were synthesised from (6S,9R)-4-chloro-1-fluoro-10-(4-methoxyphenyl)-6,7,8,9-tetrahydro-5H-6,9-epiminocyclohepta[c]pyridine 99-4 and (5R,8S)-4-chloro-1-fluoro-10-(4-methoxyphenyl)-6,7,8,9-tetrahydro-5H-5,8-epiminocyclohepta[c]pyridine 100-4 using a procedure essentially the same as for I-6. ¹H NMR (400 MHz, DMSO-d6) δ 8.07-8.06 (m, 1H), 4.40-4.29 (m, 1H), 3.82-3.79 (m, 1H), 2.93-2.84 (m, 1H), 2.50-2.37 (m, 1H), 2.02-1.90 (m, 2H), 1.83-1.71 (m, 1H), 1.58-1.48 (m, 1H).

Step 6: To a solution of 4,5-dichloro-2-fluoroaniline (42 mg, 0.23 mmol) and triphosgene (35 mg, 0.12 mol) in DCM (1 ml) at 0° C. was added a solution of DMAP (92 mg, 0.75 mol) in DCM (1 ml). The mixture was stirred at 0° C. for 1 h. A solution of (6S,9R)-4-chloro-1-fluoro-6,7,8,9-tetrahydro-5H-6,9-epiminocyclohepta[c]pyridine 99-5 and (5R,8S)-4-chloro-1-fluoro-6,7,8,9-tetrahydro-5H-5,8-epiminocyclohepta[c]pyridine 100-5 in DCM (1 ml) was added dropwise. The reaction mixture was concentrated in vacuo. The products were purified by prep-TLC (5% MeOH/DCM) followed by further purification by chiral SFC on a Sepiatec with UV detection by DAD at 220 nm, 40° C., 150 bar. The column was Chiralpak IH 10×250 mm, 5 um, flow rate 20 mL/min at 30% MeOH (0.1% Ammonia), 70% CO₂ to give (6S,9R)-4-Chloro-N-(4,5-dichloro-2-fluorophenyl)-1-fluoro-6,7,8,9-tetrahydro-5H-6,9-epiminocyclohepta[c]pyridine-10-carboxamide 99 as a colourless solid and (5R,8S)-4-Chloro-N-(4,5-dichloro-2-fluorophenyl)-1-fluoro-6,7,8,9-tetrahydro-5H-5,8-epiminocyclohepta[c]pyridine-10-carboxamide 100 as a colourless solid

Compound 99 LCMS (Method 1) m/z 415.8, 418.2 (M−H)⁻ (ES⁻), at 1.70 min, ¹H NMR (500 MHz, DMSO-d6) δ 8.97 (s, 1H), 8.17 (d, J=1.1 Hz, 1H), 7.83 (d, J=7.6 Hz, 1H), 7.66 (d, J=10.3 Hz, 1H), 5.31 (d, J=6.3 Hz, 1H), 4.78 (t, J=6.4 Hz, 1H), 3.22 (dd, J=18.6, 5.1 Hz, 1H), 2.70 (d, J=18.4 Hz, 1H), 2.34-2.24 (m, 1H), 2.18 (tt, J=11.9, 6.1 Hz, 1H), 1.97-1.89 (m, 1H), 1.76 (dt, J=15.0, 7.4 Hz, 1H).

Compound 100 LCMS (Method 1) m/z 416.1, 418.2 (M−H)⁻ (ES⁻), at 1.70 min. ¹H NMR (500 MHz, DMSO-d6) δ 8.15 (s, 1H), 7.83 (d, J=7.6 Hz, 1H), 7.66 (d, J=10.3 Hz, 1H), 5.37 (d, J=6.5 Hz, 1H), 4.79 (t, J=6.3 Hz, 1H), 3.21 (dd, J=17.6, 4.8 Hz, 1H), 2.60 (d, J=17.5 Hz, 1H), 2.38-2.12 (m, 2H), 1.81 (ddd, J=31.2, 11.5, 6.9 Hz, 2H).

### Experimental Scheme 7

### Compound 104: (5R,8S)—N-(4,5-Dichloro-2-fluorophenyl)-1-fluoro-4-hydroxy-6,7,8,9-tetrahydro-5H-5,8-epiminocyclohepta[c]pyridine-10-carboxamide

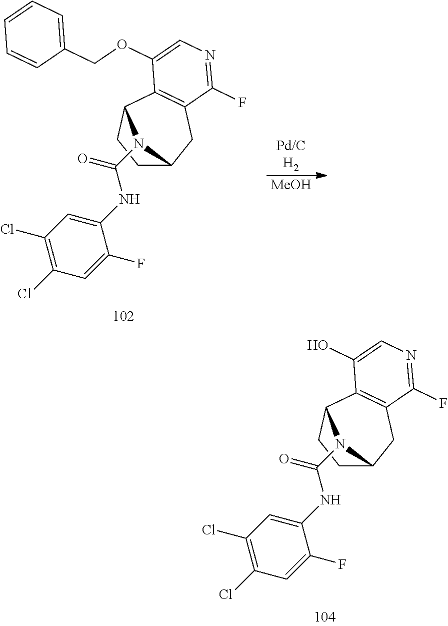 [SMILES: "MeOH" ; O=C(Nc1cc(Cl)c(Cl)cc1F)N1[C@H]2CC[C@@H]1c1c(OCc3ccccc3)cnc(F)c1C2 ; O=C(Nc1cc(Cl)c(Cl)cc1F)N1[C@H]2CC[C@@H]1c1c(O)cnc(F)c1C2]

A mixture of (5R,8S)-4-(benzyloxy)-N-(4,5-dichloro-2-fluorophenyl)-1-fluoro-6,7,8,9-tetrahydro-5H-5,8-epiminocyclohepta[c]pyridine-10-carboxamide 102 (70 mg, 0.14 mmol) and Pd/C (15 mg, 0.14 mmol) in MeOH (8 ml) was stirred for 15 h under an atmosphere of H₂. The reaction mixture was filtered through celite, the celite pad was washed with MeOH (5 ml) and the filtrate was concentrated in vacuo. The product was purified by prep-HPLC to afford (5R,8S)—N-(4,5-dichloro-2-fluorophenyl)-1-fluoro-4-hydroxy-6,7,8,9-tetrahydro-5H-5,8-epiminocyclohepta[c]pyridine-10-carboxamide 104 as a white solid. LCMS (Method 3) m/z 400.1 (M+H)⁺ (ES⁺), at 3.24 min. ¹H NMR (400 MHz, DMSO-d6) δ 10.06 (s, 1H), 8.82 (s, 1H), 7.84 (d, J=7.2 Hz, 1H), 7.66 (d, J=10 Hz, 1H), 7.50 (s, 1H), 5.36 (d, J=6.4 Hz, 1H), 4.73 (t, J=12.4 Hz, 1H), 3.17 (dd, J=17.5, 4.8 Hz, 1H), 2.52 (d, J=8.0 Hz, 1H), 2.29-2.09 (m, 2H), 1.81-1.67 (m, 2H).

### Experimental Scheme 8

### Compound 105: (5R,8S)—N-(4,5-Dichloro-2-fluorophenyl)-1-fluoro-4-(3-methoxypropoxy)-6,7,8,9-tetrahydro-5H-5,8-epiminocyclohepta[c]pyridine-10-carboxamide

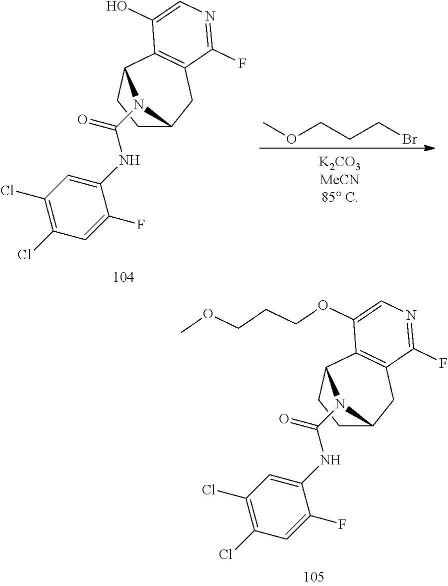

To a mixture of (5R,8S)—N-(4,5-dichloro-2-fluorophenyl)-1-fluoro-4-hydroxy-6,7,8,9-tetrahydro-5H-5,8-epiminocyclohepta[c]pyridine-10-carboxamide 104 (100 mg, 0.25 mmol) and K₂CO₃ (69.1 mg, 0.5 mmol) in MeCN (8 ml) was added 1-bromo-3-methoxypropane (58.1 mg, 0.38 mmol). The reaction was heated at 85° C. for 16 h, then diluted with water and extracted with EtOAc (3×10 ml). The combined organics were washed with brine, dried over Na₂SO₄ and concentrated in vacuo. The product was purified by prep-HPLC to afford (5R,8S)—N-(4,5-dichloro-2-fluorophenyl)-1-fluoro-4-(3-methoxypropoxy)-6,7,8,9-tetrahydro-5H-5,8-epiminocyclohepta[c]pyridine-10-carboxamide 105 as a colorless oil. LCMS (Method 3) m/z 472.1 (M+H)⁺ (ES⁺), at 1.89 min. ¹H NMR (400 MHz, DMSO-d6) δ 8.85 (s, 1H), 7.85 (d, J=7.6 Hz, 1H), 7.71 (s, 1H), 7.66 (d, J=10.4 Hz, 1H), 5.37 (d, J=6.4 Hz, 1H), 4.75 (t, J=12.8 Hz, 1H), 4.15 (t, J=12.0 Hz, 2H), 3.51 (t, J=12.4 Hz, 2H), 3.30 (s, 3H), 3.32-3.14 (dd, J=4.8, 4.8 Hz, 1H), 2.55 (d, J=20 Hz, 1H), 2.30-2.14 (m, 2H), 2.00-1.94 (m, 2H), 1.80-1.69 (m, 2H).

### Experimental Scheme 10

### Compound 107: (5R,8S)—N-(4,5-Dichloro-2-fluorophenyl)-1-fluoro-4-(3-hydroxyprop-1-yn-1-yl)-6,7,8,9-tetrahydro-5H-5,8-epiminocyclohepta[c]pyridine-10-carboxamide

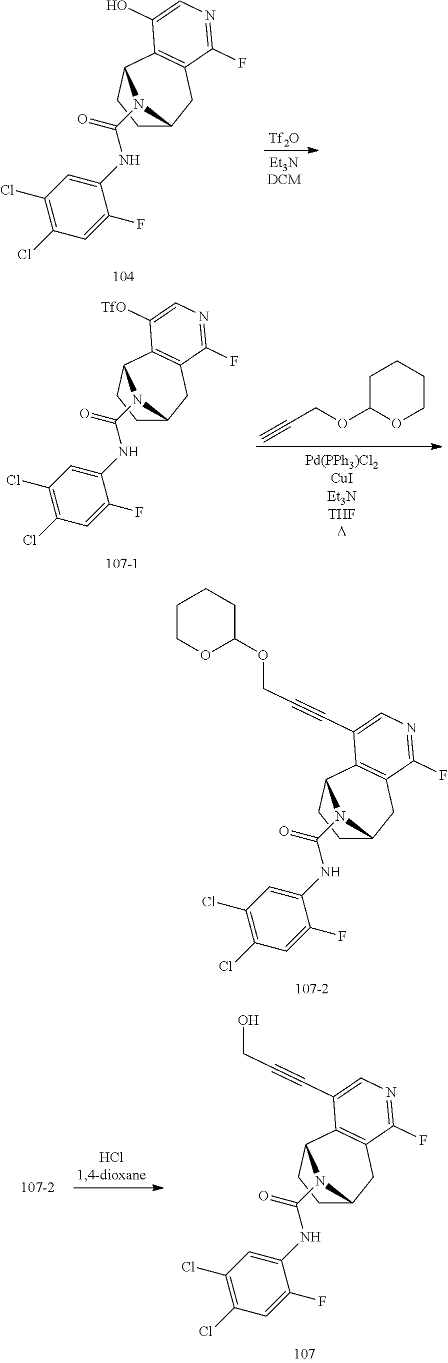

Step 1: To a solution of (5R,8S)—N-(4,5-dichloro-2-fluorophenyl)-1-fluoro-4-hydroxy-6,7,8,9-tetrahydro-5H-5,8-epiminocyclohepta[c]pyridine-10-carboxamide (450 mg, 1.12 mmol) and Et₃N (1.3 ml, 5.6 mmol) in DCM (15 ml) at 0° C. under N₂ was added Tf₂O (566.7 mg, 3.36 mmol). The mixture was stirred at 0° C. for 2 h then the mixture was diluted with water (30 ml) and extracted with DCM (3×30 ml). The combined organics were washed with brine, dried over Na₂SO₄ and concentrated in vacuo. The product was purified by prep-TLC (20% EtOAc/petroleum ether) to give (5R,8S)-10-((4,5-dichloro-2-fluorophenyl)carbamoyl)-1-fluoro-6,7,8,9-tetrahydro-5H-5,8-epiminocyclohepta[c]pyridin-4-yltrifluoromethanesulfonate 107-1 as a white solid. LCMS (Method 5) m/z 531.9 (M+H)⁺ (ES⁺), at 2.81 min.

Step 2: A mixture of (5R,8S)-10-((4,5-dichloro-2-fluorophenyl)carbamoyl)-1-fluoro-6,7,8,9-tetrahydro-5H-5,8-epiminocyclohepta[c]pyridin-4-yltrifluoromethanesulfonate 107-1 (96 mg, 0.18 mmol), 2-(prop-2-yn-1-yloxy)tetrahydro-2H-pyran (38 mg, 0.27 mmol), Pd(PPh₃)Cl₂ (12.6 mg, 0.018 mmol), CuI (6.9 mg, 0.036 mmol) and triethylamine (250 μl, 1.8 mmol) in THF (10 ml) was heated at 80° C. under N₂ for 16 h. The reaction mixture was diluted with water (15 ml) and the product was extracted with EtOAc (3×10 ml). The combined organics were washed with brine, dried over Na₂SO₄ and concentrated in vacuo. The product was purified by prep-TLC (25% EtOAc/petroleum ether) to give (5R,8S)—N-(4,5-dichloro-2-fluorophenyl)-1-fluoro-4-(3-((tetrahydro-2H-pyran-2-yl)oxy)prop-1-yn-1-yl)-6,7,8,9-tetrahydro-5H-5,8-epiminocyclohepta[c]pyridine-10-carboxamide 107-2 as a yellow oil. LCMS (Method 5) m/z 522.2 (M+H)⁺ (ES⁺), at 2.14 min.

Step 3: To a solution of (5R,8S)—N-(4,5-dichloro-2-fluorophenyl)-1-fluoro-4-(3-((tetrahydro-2H-pyran-2-yl)oxy)prop-1-yn-1-yl)-6,7,8,9-tetrahydro-5H-5,8-epiminocyclohepta[c]pyridine-10-carboxamide 107-2 (28 mg, 0.05 mmol) in 1,4-dioxane (3 ml) was added a solution of HCl in 1,4-dioxane (1 ml, 4 M, 4 mmol). The mixture was stirred for 2 h at RT then diluted with saturated aqueous NaHCO₃ solution and the product was extracted with DCM (2×20 ml). The combined organics were dried over Na₂SO₄ and concentrated in vacuo. The product was purified by prep-TLC (50% EtOAc/petroleum ether) to (5R,8S)—N-(4,5-dichloro-2-fluorophenyl)-1-fluoro-4-(3-hydroxyprop-1-yn-1-yl)-6,7,8,9-tetrahydro-5H-5,8-epiminocyclohepta[c]pyridine-10-carboxamide as a white solid. LCMS (Method 3) m/z 438.0, (M+H)⁺ (ES⁺), at 3.35 min. ¹H NMR (400 MHz, DMSO-d6) δ 8.08 (s, 1H), 7.80 (dd, J=7.6, 3.7 Hz, 1H), 7.63 (d, J=10.3 Hz, 1H), 5.33 (d, J=6.2 Hz, 1H), 4.76 (s, 1H), 4.34 (d, J=5.7 Hz, 2H), 3.21-3.11 (m, 1H), 2.54 (d, J=17.5 Hz, 2H), 2.36-2.09 (m, 2H), 1.76 (q, J=8.9 Hz, 2H).

### Experimental Scheme 11

### Compound 114: (6S,9R)—N-(5-Chloro-2-fluoro-4-(5-methoxypyridin-3-yl)phenyl)-3-oxo-3,5,6,7,8,9-hexahydro-2H-6,9-epiminocyclohepta[c]pyridazine-10-carboxamide

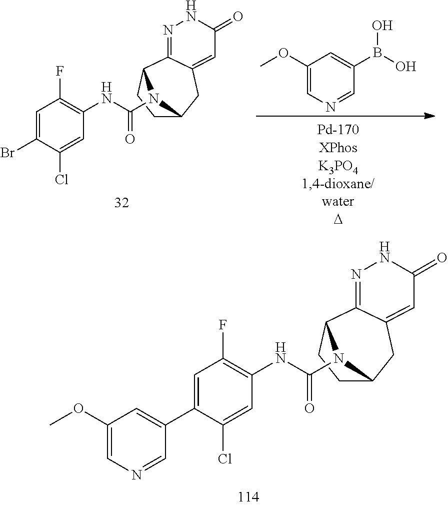 [SMILES: O=C(Nc1cc(Cl)c(Br)cc1F)N1[C@H]2CC[C@@H]1c1n[nH]c(=O)cc1C2 ; COc1cncc(B(O)O)c1 ; COc1cncc(-c2cc(F)c(NC(=O)N3[C@H]4CC[C@@H]3c3n[nH]c(=O)cc3C4)cc2Cl)c1]

To a vial was added (6S,9R)—N-(4-bromo-5-chloro-2-fluorophenyl)-3-oxo-3,5,6,7,8,9-hexahydro-2H-6,9-epiminocyclohepta[c]pyridazine-10-carboxamide 32 (20.0 mg, 46.8 μmol), Pd-170 (1.58 mg, 2.34 μmol) and XPhos (1.11 mg, 2.34 μmol) and (5-methoxypyridin-3-yl)boronic acid (9.30 mg, 60.8 μmol). The vial was purged with N₂. 1,4-Dioxane (1.00 ml) and a solution of K₃PO₄ (25 mg, 117 μmol) in water (0.20 ml) was added. The reaction mixture was heated to 80° C. for 1 h. The reaction mixture was cooled, filtered and the filtrate was concentrated in vacuo. The product was purified by mass directed reverse phase chromatography (45-100% MeCN/3% NH₃ in water) to give (6S,9R)—N-(5-chloro-2-fluoro-4-(5-methoxypyridin-3-yl)phenyl)-3-oxo-3,5,6,7,8,9-hexahydro-2H-6,9-epiminocyclohepta[c]pyridazine-10-carboxamide 114 as a colourless solid. LMS (Method 1) m/z 456.2, 458.2 (M+H)⁺ (ES)⁺ at 0.99 min. ¹H NMR (400 MHz, DMSO-d6) b 12.72 (s, 1H), 8.91 (s, 1H), 8.33 (d, J=2.8 Hz, 1H), 8.23 (d, J=1.8 Hz, 1H), 7.80 (d, J=7.3 Hz, 1H), 7.48-7.40 (m, 2H), 6.69 (s, 1H), 5.09 (d, J=5.7 Hz, 1H), 4.65 (s, 1H), 3.87 (s, 3H), 3.20 (d, J=18.9 Hz, 1H), 2.66 (d, J=18.1 Hz, 1H), 2.20 (d, J=6.4 Hz, 2H), 1.80 (t, J=9.4 Hz, 1H), 1.69 (d, J=11.8 Hz, 1H).

The following compounds were prepared using appropriate starting materials in an analogous procedure to that described in Experimental Scheme 11

| | | Method 1 | |
|---|---|---|---|
| | | LCMS Rt | |
| Compound | Structure | [M + H]⁺ | NMR |
| 117 | 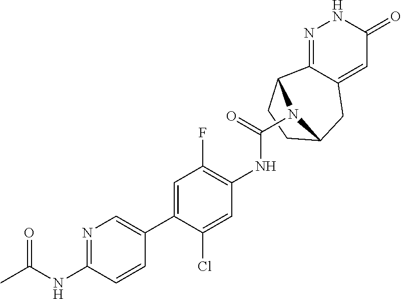 [SMILES: CC(=O)Nc1ccc(-c2cc(F)c(NC(=O)N3[C@H]4CC[C@@H]3c3n[nH]c(=O)cc3C4)cc2Cl)cn1] | 483.3, 485.2 at 0.87 min | |
| | (6S,9R)-N-(4-(6-Acetamidopyridin- | | |
| | 3-yl)-5-chloro-2-fluorophenyl)-3- | | |
| | oxo-3,5,6,7,8,9-hexahydro-2H-6,9- | | |
| | epiminocyclohepta[c]pyridazine-10- | | |
| | carboxamide | | |
| 118 | 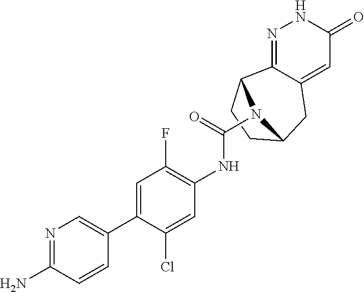 [SMILES: Nc1ccc(-c2cc(F)c(NC(=O)N3[C@H]4CC[C@@H]3c3n[nH]c(=O)cc3C4)cc2Cl)cn1] | 441.1, 443.2 at 0.81 min | |
| | (6S,9R)-N-(4-(6-Aminopyridin-3-yl)- | | |
| | 5-chloro-2-fluorophenyl)-3-oxo- | | |
| | 3,5,6,7,8,9-hexahydro-2H-6,9- | | |
| | epiminocyclohepta[c]pyridazine-10- | | |
| | carboxamide | | |
| 121 | 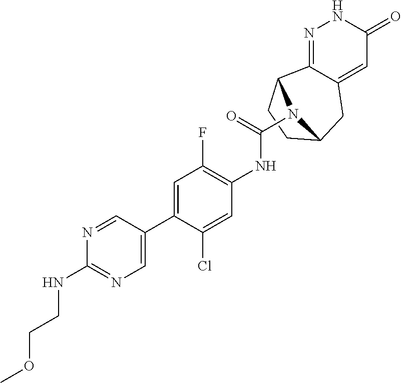 [SMILES: COCCNc1ncc(-c2cc(F)c(NC(=O)N3[C@H]4CC[C@@H]3c3n[nH]c(=O)cc3C4)cc2Cl)cn1] | 500.2, 502.2 at 0.93 min | |
| | (6S,9R)-N-(5-Chloro-2-fluoro-4-(2- | | |
| | ((2-methoxyethyl)amino)pyrimidin- | | |
| | 5-yl)phenyl)-3-oxo-3,5,6,7,8,9- | | |
| | hexahydro-2H-6,9- | | |
| | epiminocyclohepta[c]pyridazine-10- | | |
| | carboxamide | | |
| 122 | 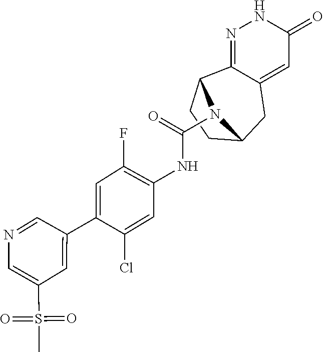 [SMILES: CS(=O)(=O)c1cncc(-c2cc(F)c(NC(=O)N3[C@H]4CC[C@@H]3c3n[nH]c(=O)cc3C4)cc2Cl)c1] | 504.2, 506.2 at 0.86 min | |
| | (6S,9R)-N-(5-Chloro-2-fluoro-4-(5- | | |
| | (methylsulfonyl)pyridin-3- | | |
| | yl)phenyl)-3-oxo-3,5,6,7,8,9- | | |
| | hexahydro-2H-6,9- | | |
| | epiminocyclohepta[c]pyridazine-10- | | |
| | carboxamide | | |
| 123 | 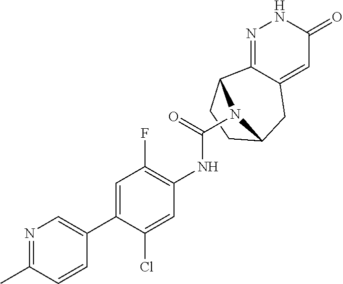 [SMILES: Cc1ccc(-c2cc(F)c(NC(=O)N3[C@H]4CC[C@@H]3c3n[nH]c(=O)cc3C4)cc2Cl)cn1] | 440.2, 442.2 at 0.98 min | |
| | (6S,9R)-N-(5-Chloro-2-fluoro-4-(6- | | |
| | methylpyridin-3-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 124 | 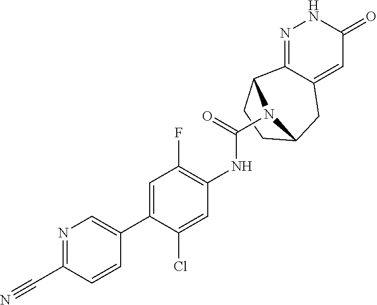 [SMILES: N#Cc1ccc(-c2cc(F)c(NC(=O)N3[C@H]4CC[C@@H]3c3n[nH]c(=O)cc3C4)cc2Cl)cn1] | 449.2, 451.2 at 1.00 min | |
| | (6S,9R)-N-(5-Chloro-4-(6- | | |
| | cyanopyridin-3-yl)-2-fluorophenyl)- | | |
| | 3-oxo-3,5,6,7,8,9-hexahydro-2H- | | |
| | 6,9-epiminocyclohepta- | | |
| | [c]pyridazine-10-carboxamide | | |
| 125 | 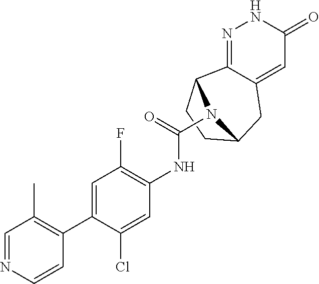 [SMILES: Cc1cnccc1-c1cc(F)c(NC(=O)N2[C@H]3CC[C@@H]2c2n[nH]c(=O)cc2C3)cc1Cl] | 440.2, 442.2 at 0.93 min | |
| | (6S,9R)-N-(5-Chloro-2-fluoro-4-(3- | | |
| | methylpyridin-4-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 126 | 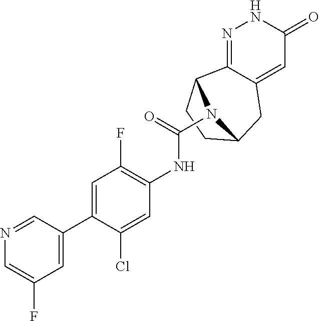 [SMILES: O=C(Nc1cc(Cl)c(-c2cncc(F)c2)cc1F)N1[C@H]2CC[C@@H]1c1n[nH]c(=O)cc1C2] | 444.2, 446.0 at 1.00 min | ¹H NMR (400 MHz, DMSO-d6) δ 12.73 (s, 1H), 8.94 (s, 1H), 8.64 (d, J = 2.7 Hz, 1H), 8.54 (t, J = 1.8 Hz, 1H), 7.90 (ddd, J = 9.9, 2.8, 1.8 Hz, 1H), 7.83 (d, J = 7.3 Hz, 1H), 7.49 (d, J = 11.1 Hz, 1H), 6.69 (s, 1H), 5.12- 5.06 (m, 1H), 4.69-4.63 (m, 1H), 3.25-3.15 (m, 1H), 2.66 (d, J = 18.1 Hz, 1H), 2.26-2.15 (m, 2H), 1.85-1.76 (m, 1H), 1.73- 1.64 (m, 1H). |
| | (6S,9R)-N-(5-Chloro-2-fluoro-4-(5- | | |
| | fluoropyridin-3-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 127 | 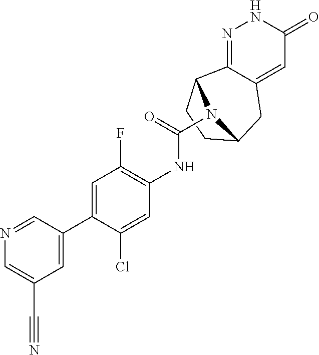 [SMILES: N#Cc1cncc(-c2cc(F)c(NC(=O)N3[C@H]4CC[C@@H]3c3n[nH]c(=O)cc3C4)cc2Cl)c1] | 451.2, 453.4 at 0.94 min | |
| | (6S,9R)-N-(5-Chloro-4-(5- | | |
| | cyanopyridin-3-yl)-2-fluorophenyl)- | | |
| | 3-oxo-3,5,6,7,8,9-hexahydro-2H- | | |
| | 6,9-epiminocyclohepta- | | |
| | [c]pyridazine-10-carboxamide | | |
| 129 | 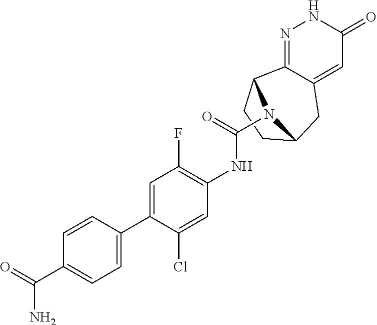 [SMILES: NC(=O)c1ccc(-c2cc(F)c(NC(=O)N3[C@H]4CC[C@@H]3c3n[nH]c(=O)cc3C4)cc2Cl)cc1] | 468.2, 470.2 at 0.81 min | |
| | (6S,9R)-N-(4′-Carbamoyl-2-chloro- | | |
| | 5-fluoro-[1,1′-biphenyl]-4-yl)-3-oxo- | | |
| | 3,5,6,7,8,9-hexahydro-2H-6,9- | | |
| | epiminocyclohepta[c]pyridazine-10- | | |
| | carboxamide | | |
| 130 | 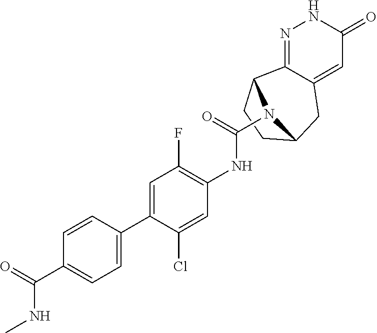 [SMILES: CNC(=O)c1ccc(-c2cc(F)c(NC(=O)N3[C@H]4CC[C@@H]3c3n[nH]c(=O)cc3C4)cc2Cl)cc1] | 482.2, 484.2 at 0.89 min | |
| | (6S,9R)-N-(2-Chloro-5-fluoro-4′- | | |
| | (methylcarbamoyl)-[1,1′-biphenyl]- | | |
| | 4-yl)-3-oxo-3,5,6,7,8,9-hexahydro- | | |
| | 2H-6,9-epiminocyclohepta- | | |
| | [c]pyridazine-10-carboxamide | | |
| 131 | 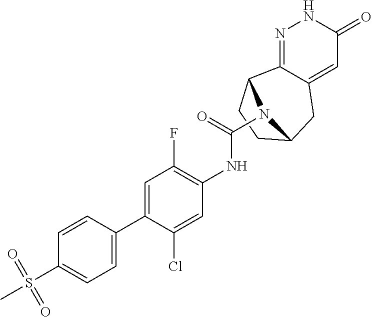 [SMILES: CS(=O)(=O)c1ccc(-c2cc(F)c(NC(=O)N3[C@H]4CC[C@@H]3c3n[nH]c(=O)cc3C4)cc2Cl)cc1] | 503.2, 505.2 at 0.96 min | |
| | (6S,9R)-N-(2-Chloro-5-fluoro-4′- | | |
| | (methylsulfonyl)-[1,1′-biphenyl]-4- | | |
| | yl)-3-oxo-3,5,6,7,8,9-hexahydro- | | |
| | 2H-6,9- | | |
| | epiminocyclohepta[c]pyridazine-10- | | |
| | carboxamide | | |
| 132 | 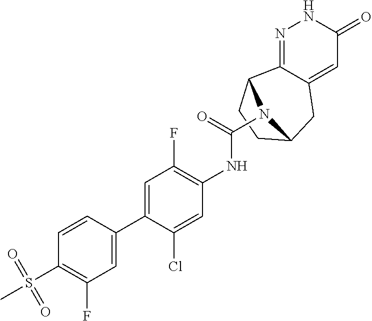 [SMILES: CS(=O)(=O)c1ccc(-c2cc(F)c(NC(=O)N3[C@H]4CC[C@@H]3c3n[nH]c(=O)cc3C4)cc2Cl)cc1F] | 521.2, 523.2 at 1.02 min | |
| | (6S,9R)-N-(2-Chloro-3′,5-difluoro- | | |
| | 4′-(methylsulfonyl)-[1,1′-biphenyl]- | | |
| | 4-yl)-3-oxo-3,5,6,7,8,9-hexahydro- | | |
| | 2H-6,9-epiminocyclohepta- | | |
| | [c]pyridazine-10-carboxamide | | |
| 133 | 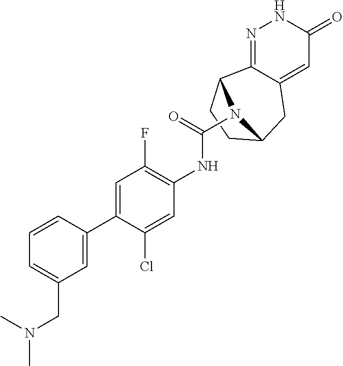 [SMILES: CN(C)Cc1cccc(-c2cc(F)c(NC(=O)N3[C@H]4CC[C@@H]3c3n[nH]c(=O)cc3C4)cc2Cl)c1] | 482.2, 484.2 at 1.21 min | |
| | (6S,9R)-N-(2-Chloro-3′- | | |
| | ((dimethylamino)methyl)-5-fluoro- | | |
| | [1,1′-biphenyl]-4-yl)-3-oxo- | | |
| | 3,5,6,7,8,9-hexahydro-2H-6,9- | | |
| | epiminocyclohepta[c]pyridazine-10- | | |
| | carboxamide | | |
| 134 | 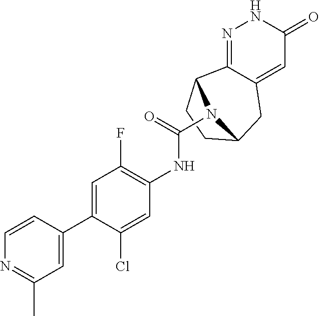 [SMILES: Cc1cc(-c2cc(F)c(NC(=O)N3[C@H]4CC[C@@H]3c3n[nH]c(=O)cc3C4)cc2Cl)ccn1] | 439.9, 442.2 at 0.95 min | ¹H NMR (400 MHz, DMSO-d6) δ 12.73 (s, 1H), 9.01 (s, 1H), 8.71 (d, J = 5.7 Hz, 1H), 7.88 (d, J = 7.2 Hz, 1H), 7.74 (s, 1H), 7.67 (s, 1H), 7.50 (d, J = 11.1 Hz, 1H), 6.69 (s, 1H), 5.09 (d, J = 5.7 Hz, 1H), 4.66 (s, 1H), 3.20 (dd, J = 17.0, 5.5 Hz, 1H), 2.72-2.60 (m, 4H), 2.19 (dd, J = 11.7, 6.3 Hz, 2H), 1.81 (t, J = 9.7 Hz, 1H), 1.69 (t, J = 8.2 Hz, 1H) |
| | (6S,9R)-N-(5-Chloro-2-fluoro-4-(2- | | |
| | methylpyridin-4-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 135 | 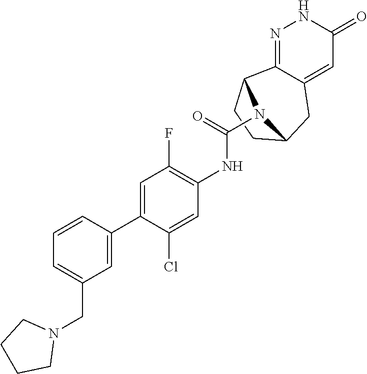 [SMILES: O=C(Nc1cc(Cl)c(-c2cccc(CN3CCCC3)c2)cc1F)N1[C@H]2CC[C@@H]1c1n[nH]c(=O)cc1C2] | 508.2, 510.2 at 1.37 min | |
| | (6S,9R)-N-(2-Chloro-5-fluoro-3′- | | |
| | (pyrrolidin-1-ylmethyl)-[1,1′- | | |
| | biphenyl]-4-yl)-3-oxo-3,5,6,7,8,9- | | |
| | hexahydro-2H-6,9- | | |
| | epiminocyclohepta[c]pyridazine-10- | | |
| | carboxamide | | |
| 136^{(a)} | 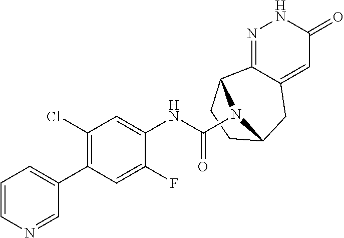 [SMILES: O=C(Nc1cc(Cl)c(-c2cccnc2)cc1F)N1[C@H]2CC[C@@H]1c1n[nH]c(=O)cc1C2] | 426.2, 428.2 at 0.88 min | ¹H NMR (400 MHz, DMSO-d6) δ 8.66-8.57 (m, 2H), 7.92-7.79 (m, 2H), 7.49 (dd, J = 7.8, 4.8 Hz, 1H), 7.42 (d, J = 11.1 Hz, 1H), 6.68 (s, 1H), 5.08 (d, J = 5.7 Hz, 1H), 4.65 (s, 1H), 3.20 (d, J = 18.0 Hz, 1H), 2.63 (s, 1H), 2.20 (d, J = 6.5 Hz, 2H), 1.82-1.73 (m, 1H), 1.69 (d, J = 10.8 Hz, 1H). (2 exchangeable protons not observed) |
| | (6S,9R)-N-(5-Chloro-2-fluoro-4- | | |
| | (pyridin-3-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 137^{(a)} | 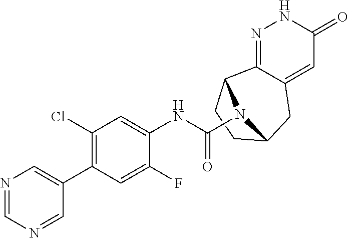 [SMILES: O=C(Nc1cc(Cl)c(-c2cncnc2)cc1F)N1[C@H]2CC[C@@H]1c1n[nH]c(=O)cc1C2] | 425.3, 427.2 at 0.73 min | ¹H NMR (400 MHz, DMSO-d6) δ 9.22 (s, 1H), 8.93 (s, 2H), 7.89 (d, J = 7.2 Hz, 1H), 7.54 (d, J = 11.2 Hz, 1H), 6.68 (s, 1H), 5.09 (d, J = 5.6 Hz, 1H), 4.65 (s, 1H), 2.62 (s, 1H), 2.20 (s, 3H), 1.80 (s, 1H), 1.67 (s, 1H). (2 exchangeable protons not observed) |
| | (6S,9R)-N-(5-Chloro-2-fluoro-4- | | |
| | (pyrimidin-5-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 138^{(a)} | 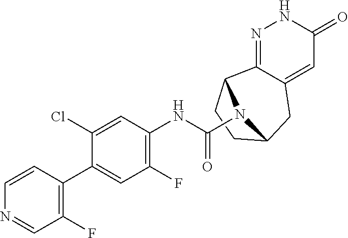 [SMILES: O=C(Nc1cc(Cl)c(-c2ccncc2F)cc1F)N1[C@H]2CC[C@@H]1c1n[nH]c(=O)cc1C2] | 444.2, 446.2 at 0.92 min | ¹H NMR (400 MHz, DMSO-d6) δ 8.69 (d, J = 1.6 Hz, 1H), 8.53 (dd, J = 4.9, 1.1 Hz, 1H), 7.88 (d, J = 7.1 Hz, 1H), 7.50 (dd, J = 6.5, 4.9 Hz, 1H), 7.45 (d, J = 11.0 Hz, 1H), 6.68 (s, 1H), 5.08 (d, J = 5.6 Hz, 1H), 4.65 (s, 1H), 3.25-3.15 (m, 1H), 2.65 (d, J = 18.3 Hz, 1H), 2.20 (d, J = 6.5 Hz, 2H), 1.84-1.75 (m, 1H), 1.68 (d, J = 11.1 Hz, 1H). (2 exchangeable protons not observed) |
| | (6S,9R)-N-(5-Chloro-2-fluoro-4-(3- | | |
| | fluoropyridin-4-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 139^{(a)} | 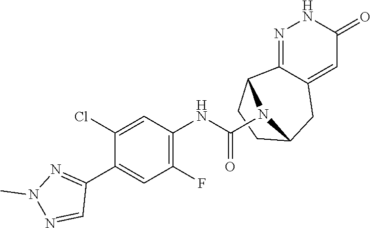 [SMILES: Cn1ncc(-c2cc(F)c(NC(=O)N3[C@H]4CC[C@@H]3c3n[nH]c(=O)cc3C4)cc2Cl)n1] | 430.2, 432.2 at 0.9 min | |
| | (6S,9R)-N-(5-Chloro-2-fluoro-4-(2- | | |
| | methyl-2H-1,2,3-triazol-4- | | |
| | yl)phenyl)-3-oxo-3,5,6,7,8,9- | | |
| | hexahydro-2H-6,9- | | |
| | epiminocyclohepta[c]pyridazine-10- | | |
| | carboxamide | | |
| 140^{(a)} | 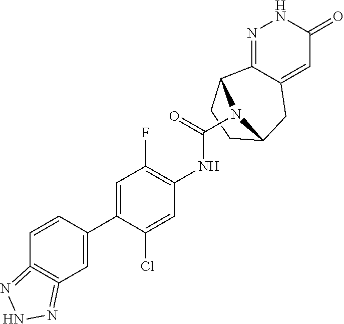 | 466.3, 468.2 at 0.58 min | |
| | (6S,9R)-N-(4-(2H- | | |
| | Benzo[d][1,2,3]triazol-5-yl)-5- | | |
| | chloro-2-fluorophenyl)-3-oxo- | | |
| | 3,5,6,7,8,9-hexahydro-2H-6,9- | | |
| | epiminocyclohepta[c]pyridazine-10- | | |
| | carboxamide | | |
| 141^{(a)} | 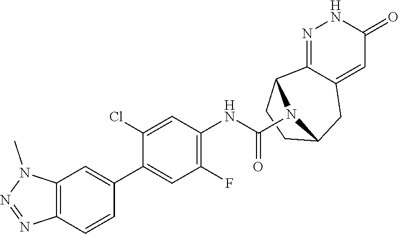 [SMILES: Cn1nnc2ccc(-c3cc(F)c(NC(=O)N4[C@H]5CC[C@@H]4c4n[nH]c(=O)cc4C5)cc3Cl)cc21] | 480.3 at 0.95 min | |
| | (6S,9R)-N-(5-Chloro-2-fluoro-4-(1- | | |
| | methyl-1H-benzo[d][1,2,3]triazol-6- | | |
| | yl)phenyl)-3-oxo-3,5,6,7,8,9- | | |
| | hexahydro-2H-6,9- | | |
| | epiminocyclohepta[c]pyridazine-10- | | |
| | carboxamide | | |
| 142^{(a)} | 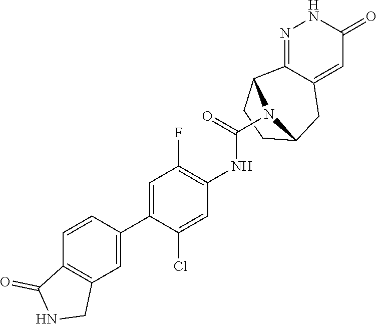 [SMILES: O=C1NCc2cc(-c3cc(F)c(NC(=O)N4[C@H]5CC[C@@H]4c4n[nH]c(=O)cc4C5)cc3Cl)ccc21] | 480.2 at 0.8 min | |
| | (6S,9R)-N-(5-Chloro-2-fluoro-4-(1- | | |
| | oxoisoindolin-5-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 143^{(a)} | 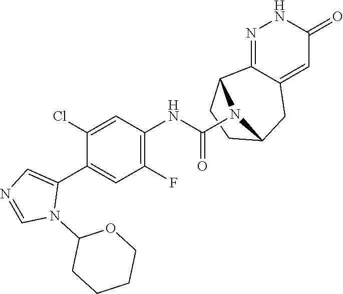 [SMILES: O=C(Nc1cc(Cl)c(-c2cncn2C2CCCCO2)cc1F)N1[C@H]2CC[C@@H]1c1n[nH]c(=O)cc1C2] | 414.9, 415.5, 417.2 at 0.89 min^{(b)} | |
| | (6S,9R)-N-(5-Chloro-2-fluoro-4-(1- | | |
| | (tetrahydro-2H-pyran-2-yl)-1H- | | |
| | imidazol-5-yl)phenyl)-3-oxo- | | |
| | 3,5,6,7,8,9-hexahydro-2H-6,9- | | |
| | epiminocyclohepta[c]pyridazine-10- | | |
| | carboxamide | | |
| 144^{(a)} | 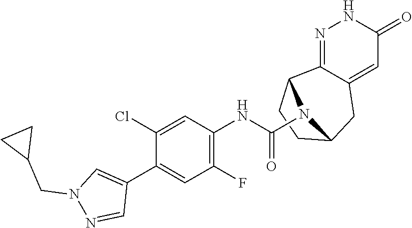 [SMILES: O=C(Nc1cc(Cl)c(-c2cnn(CC3CC3)c2)cc1F)N1[C@H]2CC[C@@H]1c1n[nH]c(=O)cc1C2] | 469.2, 471.2 at 1.05 min | ¹H NMR (400 MHz, DMSO-d6) δ 8.25 (s, 1H), 7.88 (s, 1H), 7.67 (d, J = 7.5 Hz, 1H), 7.50 (d, J = 11.9 Hz, 1H), 6.68 (s, 1H), 5.06 (d, J = 6.0 Hz, 1H), 4.65-4.60 (m, 1H), 4.00 (d, J = 7.1 Hz, 2H), 3.19 (d, J = 22.0 Hz, 1H), 2.64 (d, J = 18.1 Hz, 1H), 2.27-2.10 (m, 2H), 1.81-1.74 (m, 1H), 1.72- 1.61 (m, 1H), 1.32- 1.20 (m, 1H), 0.58-0.49 |
| | (6S,9R)-N-(5-Chloro-4-(1- | | (m, 2H), 0.42-0.34 (m, |
| | (cyclopropylmethyl)-1H-pyrazol-4- | | 2H). 2 exchangeable |
| | yl)-2-fluorophenyl)-3-oxo- | | protons not observed |
| | 3,5,6,7,8,9-hexahydro-2H-6,9- | | |
| | epiminocyclohepta[c]pyridazine-10- | | |
| | carboxamide | | |
| 145 | 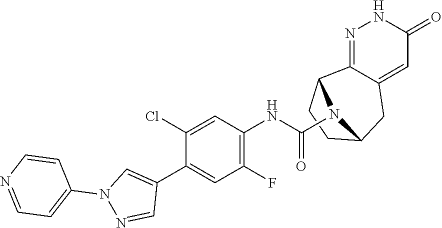 [SMILES: O=C(Nc1cc(Cl)c(-c2cnn(-c3ccncc3)c2)cc1F)N1[C@H]2CC[C@@H]1c1n[nH]c(=O)cc1C2] | 492.3, 494.3 at 0.96 min | |
| | (6S,9R)-N-(5-Chloro-2-fluoro-4-(1- | | |
| | (pyridin-4-yl)-1H-pyrazol-4- | | |
| | yl)phenyl)-3-oxo-3,5,6,7,8,9- | | |
| | hexahydro-2H-6,9- | | |
| | epiminocyclohepta[c]pyridazine-10- | | |
| | carboxamide | | |
| 146 | 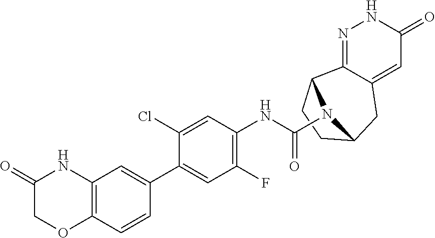 [SMILES: O=C1COc2ccc(-c3cc(F)c(NC(=O)N4[C@H]5CC[C@@H]4c4n[nH]c(=O)cc4C5)cc3Cl)cc2N1] | 496.2, 498.2 at 0.96 min | |
| | (6S,9R)-N-(5-Chloro-2-fluoro-4-(3- | | |
| | oxo-3,4-dihydro-2H- | | |
| | benzo[b][1,4]oxazin-6-yl)phenyl)-3- | | |
| | oxo-3,5,6,7,8,9-hexahydro-2H-6,9- | | |
| | epiminocyclohepta[c]pyridazine-10- | | |
| | carboxamide | | |
| 147 | 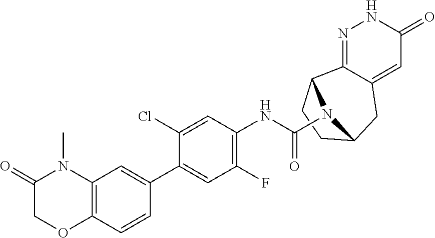 [SMILES: CN1C(=O)COc2ccc(-c3cc(F)c(NC(=O)N4[C@H]5CC[C@@H]4c4n[nH]c(=O)cc4C5)cc3Cl)cc21] | 510.2, 512.2 at 1.07 min | ¹H NMR (400 MHz, DMSO-d6) δ 7.76 (d, J = 7.4 Hz, 1H), 7.34 (d, J = 11.2 Hz, 1H), 7.19 (d, J = 1.9 Hz, 1H), 7.10 (dd, J = 8.3, 1.8 Hz, 1H), 7.07 (d, J = 8.2 Hz, 1H), 6.68 (d, J = 1.8 Hz, 1H), 5.08 (d, J = 5.7 Hz, 1H), 4.70 (s, 2H), 4.63 (d, J = 7.2 Hz, 1H), 3.30 (s, 3H), 3.20 (dd, J = 18.3, 5.4 Hz, 1H), 2.64 (d, J = 18.4 Hz, 1H), 2.24-2.11 (m, 2H), 1.85-1.75 (m, 1H), 1.73- |
| | (6S,9R)-N-(5-Chloro-2-fluoro-4-(4- | | 1.62 (m, 1H). 2 |
| | methyl-3-oxo-3,4-dihydro-2H- | | exchangeable protons |
| | benzo[b][1,4]oxazin-6-yl)phenyl)-3- | | not observed |
| | oxo-3,5,6,7,8,9-hexahydro-2H-6,9- | | |
| | epiminocyclohepta[c]pyridazine-10- | | |
| | carboxamide | | |
| 148 | 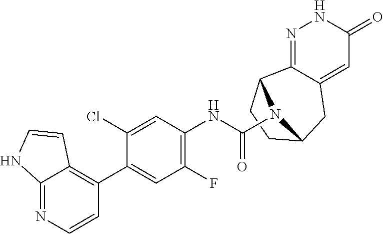 [SMILES: O=C(Nc1cc(Cl)c(-c2ccnc3[nH]ccc23)cc1F)N1[C@H]2CC[C@@H]1c1n[nH]c(=O)cc1C2] | 465.2, 467.2 at 0.89 min | |
| | (6S,9R)-N-(5-Chloro-2-fluoro-4- | | |
| | (1H-pyrrolo[2,3-b]345yridine-4- | | |
| | yl)phenyl)-3-oxo-3,5,6,7,8,9- | | |
| | hexahydro-2H-6,9- | | |
| | epiminocyclohepta[c]pyridazine-10- | | |
| | carboxamide | | |
| 149 | 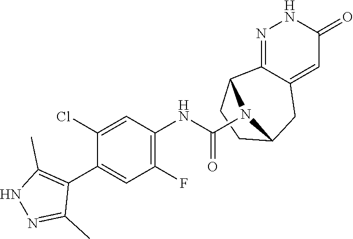 [SMILES: Cc1n[nH]c(C)c1-c1cc(F)c(NC(=O)N2[C@H]3CC[C@@H]2c2n[nH]c(=O)cc2C3)cc1Cl] | 443.3, 445.2 at 0.80 min | |
| | (6S,9R)-N-(5-Chloro-4-(3,5- | | |
| | dimethyl-1H-pyrazol-4-yl)-2- | | |
| | fluorophenyl)-3-oxo-3,5,6,7,8,9- | | |
| | hexahydro-2H-6,9- | | |
| | epiminocyclohepta[c]pyridazine-10- | | |
| | carboxamide | | |
| 150 | 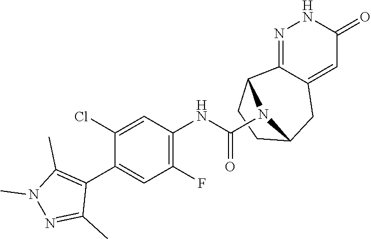 [SMILES: Cc1nn(C)c(C)c1-c1cc(F)c(NC(=O)N2[C@H]3CC[C@@H]2c2n[nH]c(=O)cc2C3)cc1Cl] | 457.0, 458.3 at 0.89 min | |
| | (6S,9R)-N-(5-Chloro-2-fluoro-4- | | |
| | (1,3,5-trimethyl-1H-pyrazol-4- | | |
| | yl)phenyl)-3-oxo-3,5,6,7,8,9- | | |
| | hexahydro-2H-6,9- | | |
| | epiminocyclohepta[c]pyridazine-10- | | |
| | carboxamide | | |
| 151 | 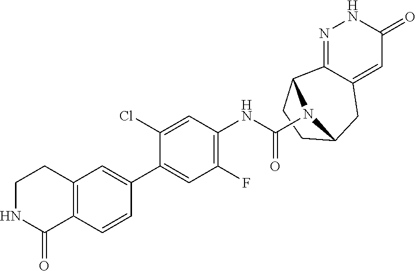 [SMILES: O=C1NCCc2cc(-c3cc(F)c(NC(=O)N4[C@H]5CC[C@@H]4c4n[nH]c(=O)cc4C5)cc3Cl)ccc21] | 494.2, 496.3 at 0.87 min | |
| | (6S,9R)-N-(5-Chloro-2-fluoro-4-(1- | | |
| | oxo-1,2,3,4-tetrahydroisoquinolin- | | |
| | 6-yl)phenyl)-3-oxo-3,5,6,7,8,9- | | |
| | hexahydro-2H-6,9- | | |
| | epiminocyclohepta[c]pyridazine-10- | | |
| | carboxamide | | |

^{(a)}Pd(II)Cl₂(dtbpf)(5 Mol %) was used instead of Pd-170
^{(b)}Mass ion is (M-tetrahydropyran)⁺

### Experimental Scheme 12

### Compound 115: (6S,9R)—N-(5-Chloro-2-fluoro-4-(2-fluoropyridin-3-yl)phenyl)-3-oxo-3,5,6,7,8,9-hexahydro-2H-6,9-epiminocyclohepta[c]pyridazine-10-carboxamide

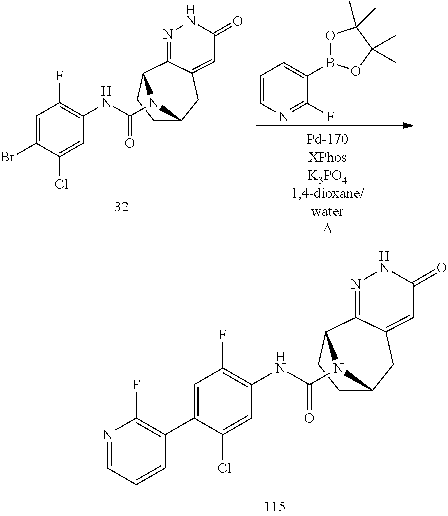 [SMILES: O=C(Nc1cc(Cl)c(Br)cc1F)N1[C@H]2CC[C@@H]1c1n[nH]c(=O)cc1C2 ; CC1(C)OB(c2cccnc2F)OC1(C)C ; O=C(Nc1cc(Cl)c(-c2cccnc2F)cc1F)N1[C@H]2CC[C@@H]1c1n[nH]c(=O)cc1C2]

(6S,9R)—N-(5-Chloro-2-fluoro-4-(2-fluoropyridin-3-yl)phenyl)-3-oxo-3,5,6,7,8,9-hexahydro-2H-6,9-epiminocyclohepta[c]pyridazine-10-carboxamide 115 was synthesised from (6S,9R)—N-(4-bromo-5-chloro-2-fluorophenyl)-3-oxo-3,5,6,7,8,9-hexahydro-2H-6,9-epiminocyclohepta[c]pyridazine-10-carboxamide 32 using a procedure essentially the same as for 114. LCMS (Method 1) m/z 444.4, 446.4 (M+H)⁺ (ES)⁺ at 1.03 min. ¹H NMR (400 MHz, DMSO-d6) δ 12.72 (s, 1H), 8.93 (s, 1H), 8.32 (d, J=5.2 Hz, 1H), 7.99 (ddd, J=9.6, 7.5, 2.0 Hz, 1H), 7.82 (d, J=7.2 Hz, 1H), 7.52-7.43 (m, 2H), 6.69 (s, 1H), 5.08 (d, J=5.6 Hz, 1H), 4.65 (s, 1H), 3.22 (s, 1H), 2.66 (d, J=17.9 Hz, 1H), 2.24-2.15 (m, 2H), 1.80 (t, J=9.5 Hz, 1H), 1.69 (d, J=11.5 Hz, 1H).

The following compounds were prepared using appropriate starting materials in an analogous procedure to that described in Experimental Scheme 12

| | | Method | |
|---|---|---|---|
| | | LCMS Rt | |
| Compound | Structure | [M + H]⁺ | NMR |
| 116 | 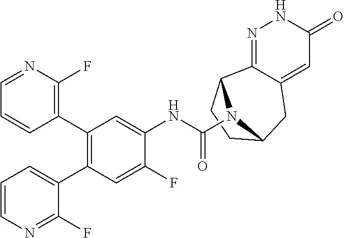 [SMILES: O=C(Nc1cc(-c2cccnc2F)c(-c2cccnc2F)cc1F)N1[C@H]2CC[C@@H]1c1n[nH]c(=O)cc1C2] | Method 1 505.2 at 0.97 min | ¹H NMR (400 MHz, DMSO-d6) δ 12.60 (s, 1H), 8.89 (s, 1H), 8.14 (d, J = 4.9 Hz, 2H), 7.83-7.71 (m, 2H), 7.65 (d, J = 7.8 Hz, 1H), 7.46 (d, J = 11.2 Hz, 1H), 7.31 (dd, J = 7.0, 5.2 Hz, 2H), 6.68 (s, 1H), 5.07 (d, J = 5.6 Hz, 1H), 4.65 (s, 1H), 3.21 (d, J = 16.1 Hz, 1H), 2.63 (s, 1H), 2.21 (d, J = 7.2 Hz, 2H), 1.79 (d, J = 10.7 Hz, 1H), 1.68 (s, 1H) |
| | (6S,9R)-N-(2-Fluoro-4,5-bis(2- | | |
| | fluoropyridin-3-yl)phenyl)-3-oxo- | | |
| | 3,5,6,7,8,9-hexahydro-2H-6,9- | | |
| | epiminocyclohepta[c]pyridazine- | | |
| | 10-carboxamide | | |

### Experimental Scheme 13

### Compound 154: (±)—N-(4,5-Dichloro-2-fluorophenyl)-1-fluoro-9-methylene-6,7,8,9-tetrahydro-5H-5,8-epiminocyclohepta[c]pyridine-10-carboxamide

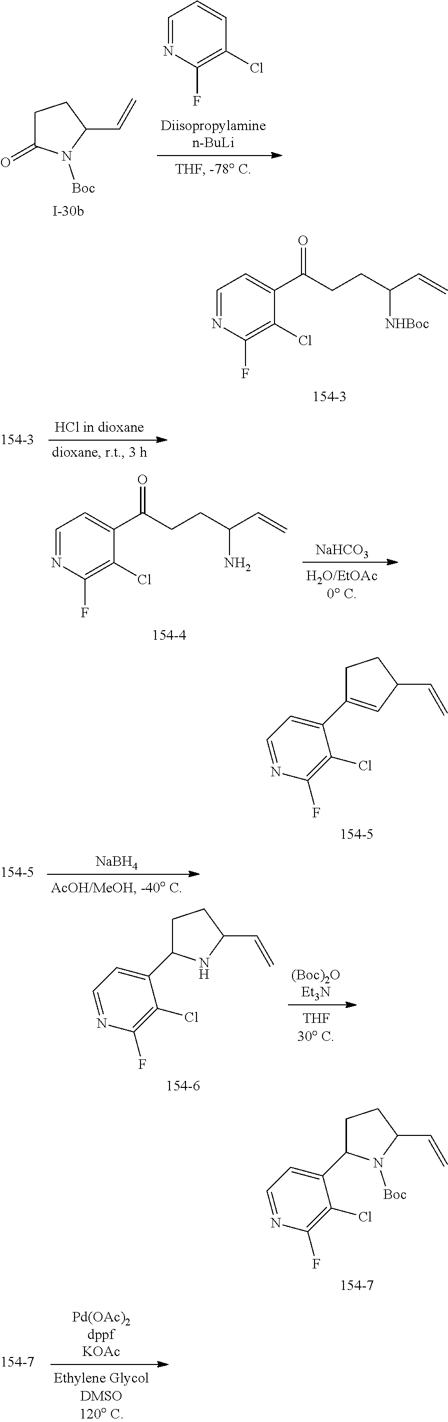

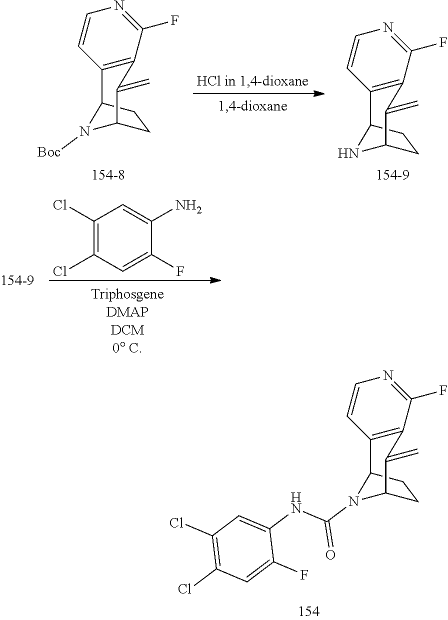 [SMILES: C=C1c2c(ccnc2F)C2CCC1N2C(=O)OC(C)(C)C ; C=C1c2c(ccnc2F)C2CCC1N2 ; Nc1cc(Cl)c(Cl)cc1F ; C=C1c2c(ccnc2F)C2CCC1N2C(=O)Nc1cc(Cl)c(Cl)cc1F]

Step 1: To a solution of diisopropylamine (27 ml, 191.7 mmol) in THF (120 ml) was added ⁿBuLi (76.7 ml, 2.5 M in hexane, 191.75 mmol) drop-wise at −78° C. After stirring at −78° C. for 1 h, a solution of 3-chloro-2-fluoropyridine (16.8 g, 127.8 mmol) in THF (100 ml) was added. The reaction solution was stirred at −78° C. for another 1 h. After that, a solution of tert-butyl 2-oxo-5-vinylpyrrolidine-1-carboxylate I-30b (27 g, 127.8 mmol, 1.0 eq) in THF (200 ml) was added and the reaction mixture was stirred at −78° C. for 1 h. The mixture was poured into saturated NH₄Cl solution (800 ml) and extracted with EtOAc (3×200 ml). The combined organics were washed with brine (500 ml), dried over Na₂SO₄ and concentrated in vacuo. The product was purified by chromatography on silica gel column (3%-33% EtOAc/petroleum ether) to give tert-butyl (6-(3-chloro-2-fluoropyridin-4-yl)-6-oxohex-1-en-3-yl)carbamate 154-3 as a yellow oil. LCMS (Method 5) m/z 343.1 (M+H)⁺ (ES)⁺ at 2.25 min.

Step 2: To a solution of tert-butyl (6-(3-chloro-2-fluoropyridin-4-yl)-6-oxohex-1-en-3-yl) carbamate 154-3 (16.7 g, 48.7 mmol) in 1,4-dioxane (80 ml) was added a solution of HCl in 1,4-dioxane (120 ml, 4 M, 480 mmol) at RT under N₂. After stirring for 3 h, the reaction mixture was concentrated in vacuo to give 4-amino-1-(3-chloro-2-fluoropyridin-4-yl)hex-5-en-1-one 154-4 as a brown oil. LCMS (Method 5) m/z 243.1 (M+H)⁺ (ES)⁺ at 1.88 min.

Step 3: To a solution of NaHCO₃ (10.2 g, 121.5 mmol) in water (50 ml) was added a solution of 4-amino-1-(3-chloro-2-fluoropyridin-4-yl) hex-5-en-1-one 154-4 (11.8 g, 48.6 mmol) in EtOAc (200 ml) at 0° C. under N₂. After stirring for 3 h, the reaction mixture was diluted with water (300 ml) and extracted with EtOAc (2×100 ml). The combined organics were washed with brine (100 ml), dried over Na₂SO₄ and concentrated in vacuo. The product was purified by chromatography on silica gel (20% EtOAc/petroleum ether) to give 3-chloro-2-fluoro-4-(2-vinyl-3,4-dihydro-2H-pyrrol-5-yl)pyridine 154-5 as a yellow oil. LCMS (Method 5) m/z 224.7 (M+H)⁺ (ES)⁺ at 1.24 min.

Step 4: To a solution of AcOH (1.6 ml) in MeOH (20 ml) was added a solution of 3-chloro-2-fluoro-4-(2-vinyl-3,4-dihydro-2H-pyrrol-5-yl)pyridine 154-5 (9.4 g, 41.8 mmol) in MeOH (60 ml) drop-wise at −40° C. After stirring at −40° C. for 1 h, NaBH₄ (3.6 g, 96.1 mmol) was added. The progress of reaction was monitored by TLC. After completion, the mixture was poured into saturated NH₄Cl solution (200 ml) and extracted with EtOAc (3×100 ml). The combined organics were washed with brine (100 ml), dried over Na₂SO₄ and concentrated in vacuo. The residue was purified by chromatography on silica gel column (3% EtOAc/petroleum ether) to give 3-chloro-2-fluoro-4-(5-vinylpyrrolidin-2-yl)pyridine 154-6 as a yellow oil. LCMS (Method 5) m/z 227.1 (M+H)⁺ (ES)⁺ at 0.56 min.

Step 5: To a solution of 3-chloro-2-fluoro-4-(5-vinylpyrrolidin-2-yl)pyridine 154-6 (1.2 g, 5.29 mmol) and Et₃N (1.47 ml, 10.58 mmol) in THF (15 ml) was added (Boc)₂O (1.73 g, 7.94 mmol). The reaction mixture was stirred at 30° C. for 16 h. The mixture was concentrated in vacuo. The product was purified by chromatography on silica gel column (10% EtOAc/petroleum ether) to give tert-butyl 2-(3-chloro-2-fluoropyridin-4-yl)-5-vinylpyrrolidine-1-carboxylate 154-7 as a yellow oil. LCMS (Method 5) m/z 327.1 (M+H)⁺ (ES)⁺ at 2.58 min.

Step 6: A mixture of tert-butyl 2-(3-chloro-2-fluoropyridin-4-yl)-5-vinylpyrrolidine-1-carboxylate 154-7 (1.7 g, 5.2 mmol), Pd(OAc)₂ (58.4 mg, 0.26 mmol), dppf (216.2 mg, 0.39 mmol) and KOAc (766 mg, 7.8 mmol) in ethylene glycol (1 ml) and DMSO-d6 (1.8 mL) was stirred at 120° C. under N₂ for 3 days. The reaction mixture was cooled to RT, diluted with water (100 ml) and the product was extracted with EtOAc (3×100 ml). The combined organics were washed with brine (100 ml), dried over Na₂SO₄ and concentrated in vacuo. The product was purified by chromatography on silica gel column (20% EtOAc/petroleum ether) to give tert-butyl (±)-1-fluoro-9-methylene-6,7,8,9-tetrahydro-5H-5,8-epiminocyclohepta[c]pyridine-10-carboxylate 154-8 as a yellow oil. ¹H NMR (400 MHz, DMSO-d6) δ 8.06 (d, J=5.2 Hz, 1H), 7.33 (d, J=6.8 Hz, 1H), 5.39-5.38 (m, 1H), 4.99 (d, J=6.4 Hz, 1H), 4.71-4.69 (m, 1H), 3.43-3.41 (m, 1H), 2.32-2.21 (m, 2H), 1.80-1.74 (m, 1H), 1.65-1.62 (m, 1H), 1.33 (s, 9H).

Step 7: To a solution of tert-butyl (±)-1-fluoro-9-methylene-6,7,8,9-tetrahydro-5H-5,8-epiminocyclohepta[c]pyridine-10-carboxylate 154-8 (60 mg, 0.21 mmol) in 1,4-dioxane (3 ml) was added a solution of HCl in 1,4-dioxane (2.5 ml, 4 M) at RT under N₂. After stirring for 2 h, the reaction mixture was basified with saturated NaHCO₃ aqueous solution and extracted with DCM (3×10 ml). The combined organics were dried over Na₂SO₄ and concentrated in vacuo to give (±)-1-fluoro-9-methylene-6,7,8,9-tetrahydro-5H-5,8-epiminocyclohepta[c]pyridine 154-9 as a yellow oil. LCMS (Method 5) m/z 191.2 (M+H)⁺ (ES)⁺ at 0.58 min.

Step 8: To a solution of 4,5-dichloro-2-fluoroaniline (37.8 mg, 0.21 mmol) and triphosgene (32.6 mg, 0.11 mmol) in DCM (4 ml) at 0° C. was added a solution of DMAP (81.9 mg, 0.67 mol) in DCM (1 ml). The mixture was stirred at 0° C. for 30 min. This was added to a solution of (±)-1-fluoro-9-methylene-6,7,8,9-tetrahydro-5H-5,8-epiminocyclohepta[c]pyridine 154-9 (40 mg, 0.21 mol) in DCM (2 ml). The reaction mixture was stirred at RT for 16 h. the reaction mixture was poured into water and extracted with DCM (3×10 ml). The combined organics were washed with brine, dried over Na₂SO₄ and concentrated in vacuo. The product was purified by prep-TLC (33% EtOAc/petroleum ether) to give (±)—N-(4,5-dichloro-2-fluorophenyl)-1-fluoro-9-methylene-6,7,8,9-tetrahydro-5H-5,8-epiminocyclohepta[c]pyridine-10-carboxamide 154 as a white solid. LCMS (Method 3) m/z 394.0 (M+H)⁺ (ES)⁺ at 3.49 min. ¹H NMR (400 MHz, DMSO-d6) δ 8.80 (s, 1H), 8.07 (d, J=4.8 Hz, 1H), 7.77 (d, J=7.6 Hz, 1H), 7.67 (d, J=10.4 Hz, 1H), 7.32 (d, J=6.4 Hz, 1H), 5.80 (s, 1H), 5.54 (d, J=4.8 Hz, 1H), 5.26 (d, J=6.4 Hz, 1H), 5.13 (d, J=7.6 Hz, 1H), 2.43-2.40 (m, 1H), 2.32-2.26 (m, 1H), 1.86-1.80 (m, 1H), 1.75-1.70 (m, 1H).

### Experimental Scheme 14

### Compound 155: (±)—N-(4,5-Dichloro-2-fluorophenyl)-1-fluoro-9-oxo-6,7,8,9-tetrahydro-5H-5,8-epiminocyclohepta[c]pyridine-10-carboxamide

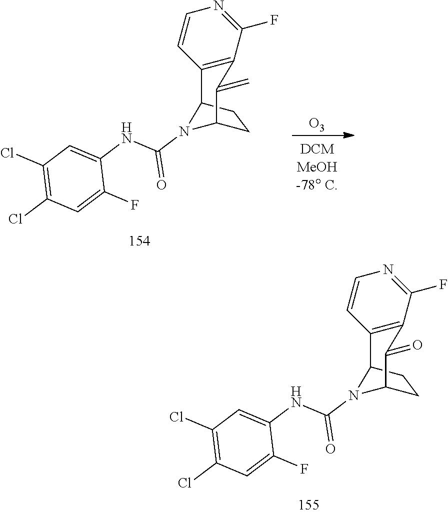

To a solution of N-(4,5-dichloro-2-fluorophenyl)-1-fluoro-9-methylene-6,7,8,9-tetrahydro-5H-5,8-epiminocyclohepta[c]pyridine-10-carboxamide 154 (20 mg, 0.05 mmol) in DCM:MeOH (10:1, 4 ml) at −78° C. with stirring was bubbled O₃ at −78° C. for 10 min. After this time a drop of dimethyl sulfur was added into the reaction mixture. The mixture was concentrated in vacuo. The residue was diluted with DCM (5 ml) and the mixture was filtered. The filter cake was washed with DCM (10 ml) and the filtrate was concentrated in vacuo. The product was purified by prep-TLC (50% EtOAc/petroleum ether) to give (±)—N-(4,5-dichloro-2-fluorophenyl)-1-fluoro-9-oxo-6,7,8,9-tetrahydro-5H-5,8-epiminocyclohepta[c]pyridine-10-carboxamide 155 as a white solid. LCMS (Method 3) m/z 396.1 (M+H)⁺ (ES)⁺ at 3.08 min. ¹H NMR (400 MHz, DMSO-d6) δ 9.18 (s, 1H), 8.44 (d, J=5.2 Hz, 1H), 7.70 (d, J=7.2 Hz, 1H), 7.66 (d, J=10.0 Hz, 1H), 7.53 (d, J=5.2 Hz, 1H), 5.53 (d, J=6.0 Hz, 1H), 4.83 (d, J=7.6 Hz, 1H), 2.45-2.41 (m, 2H), 1.87-1.80 (m, 2H).

### Experimental Scheme 15

### Compound 156: (±)-(9S)—N-(5-chloro-2-fluoro-4-(trifluoromethyl)phenyl)-1-fluoro-9-hydroxy-6,7,8,9-tetrahydro-5H-5,8-epiminocyclohepta[c]pyridine-10-carboxamide

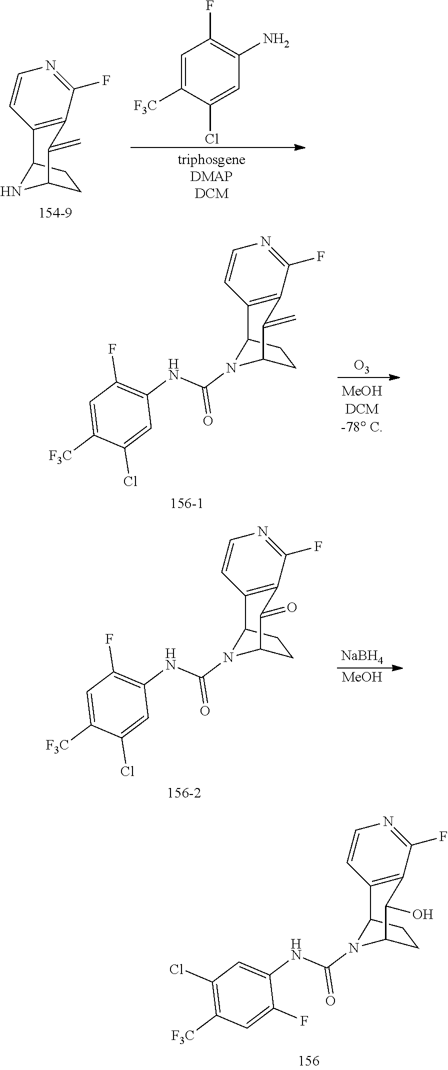

Step 1: (±)—N-(5-Chloro-2-fluoro-4-(trifluoromethyl)phenyl)-1-fluoro-9-methylene-6,7,8,9-tetrahydro-5H-5,8-epiminocyclohepta[c]pyridine-10-carboxamide 156-1 was synthesised from (±)-1-fluoro-9-methylene-6,7,8,9-tetrahydro-5H-5,8-epiminocyclohepta[c]pyridine 154-9 and 5-chloro-2-fluoro-4-(trifluoromethyl)aniline using a procedure essentially the same as for 154. LCMS (Method 5) m/z 429.6 (M+H)⁺ (ES)⁺ at 1.95 min. ¹H NMR (400 MHz, DMSO-d6) δ 9.08 (s, 1H), 8.08 (d, J=6.0 Hz, 1H), 7.97 (d, J=6.8 Hz, 1H), 7.78 (d, J=11.2 Hz, 1H), 7.34 (d, J=6.4 Hz, 1H), 5.80 (s, 1H), 5.44 (d, J=4.4 Hz, 1H), 5.30 (d, J=6.8 Hz, 1H), 5.19 (d, J=7.2 Hz, 1H), 2.44-2.41 (m, 1H), 2.32-2.27 (m, 1H), 1.87-1.82 (m, 1H), 1.76-1.71 (m, 1H).

Step 2: (±)—N-(5-Chloro-2-fluoro-4-(trifluoromethyl)phenyl)-1-fluoro-9-oxo-6,7,8,9-tetrahydro-5H-5,8-epiminocyclohepta[c]pyridine-10-carboxamide 156-2 was synthesised from (±)—N-(5-chloro-2-fluoro-4-(trifluoromethyl)phenyl)-1-fluoro-9-methylene-6,7,8,9-tetrahydro-5H-5,8-epiminocyclohepta[c]pyridine-10-carboxamide 156-1, using a procedure essentially the same as for 155. LCMS (Method 5) m/z 431.9 (M+H)⁺ (ES)⁺ at 1.66 min. ¹H NMR (400 MHz, DMSO-d6) δ 9.45 (s, 1H), 8.44 (d, J=4.8 Hz, 1H), 7.90 (d, J=6.8 Hz, 1H), 7.78 (d, J=10.8 Hz, 1H), 7.56 (d, J=6.0 Hz, 1H), 5.58 (d, J=6.0 Hz, 1H), 4.88 (d, J=7.6 Hz, 1H), 2.47-2.42 (m, 2H), 1.87-1.81 (m, 2H).

Step 3: To a solution of (±)—N-(5-chloro-2-fluoro-4-(trifluoromethyl)phenyl)-1-fluoro-9-oxo-6,7,8,9-tetrahydro-5H-5,8-epiminocyclohepta[c]pyridine-10-carboxamide 156-2 (110 mg, 0.25 mmol) in MeOH (10 ml) was added NaBH₄ (19 mg, 0.5 mol) under N₂ atmosphere. The reaction mixture was stirred at RT for 1 h. After completion, the reaction mixture was concentrated in vacuo. The product was purified by prep-TLC (50% EtOAc/petroleum ether) to give (±)-(9S)—N-(5-chloro-2-fluoro-4-(trifluoromethyl)phenyl)-1-fluoro-9-hydroxy-6,7,8,9-tetrahydro-5H-5,8-epiminocyclohepta[c]pyridine-10-carboxamide 156 as a white solid. LCMS (Method 3) m/z 433.9 (M+H)⁺ (ES)⁺ at 3.44 min. ¹H NMR (400 MHz, DMSO-d6) δ 9.15 (s, 1H), 8.05 (d, J=4.8 Hz, 1H), 8.01 (d, J=6.8 Hz, 1H), 7.78 (d, J=10.8 Hz, 1H), 7.20 (d, J=6.8 Hz, 1H), 5.89 (d, J=6.4 Hz, 1H), 5.25-5.20 (m, 2H), 4.65 (t, J=13.2 Hz, 1H), 2.34-2.31 (m, 1H), 2.17-2.13 (m, 1H), 1.98-1.95 (m, 1H), 1.78-1.72 (m, 1H).

### Experimental Scheme 16

### Compound 157: (±)-(9R)—N-(5-Chloro-2-fluoro-4-(trifluoromethyl)phenyl)-1,9-difluoro-6,7,8,9-tetrahydro-5H-5,8-epiminocyclohepta[c]pyridine-10-carboxamide

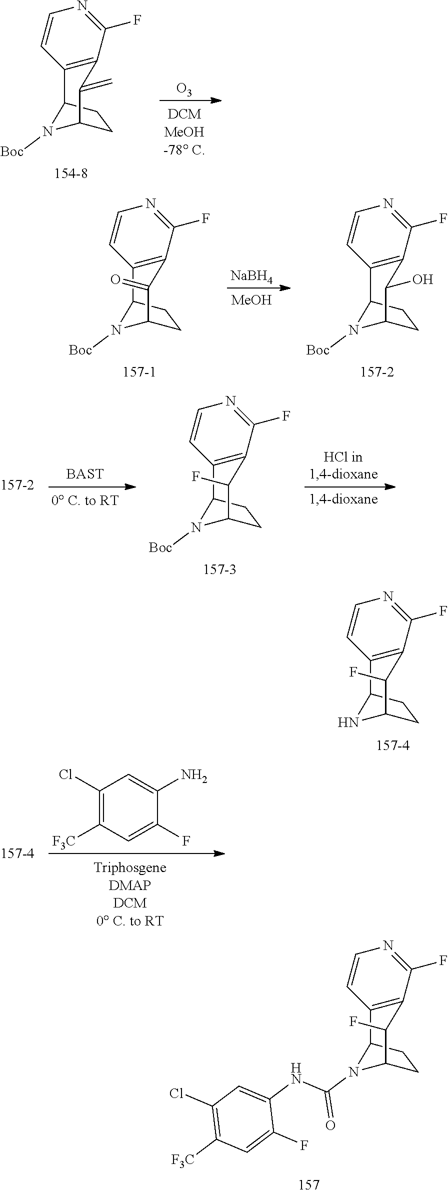

Step 1: tert-Butyl 1-fluoro-9-oxo-6,7,8,9-tetrahydro-5H-5,8-epiminocyclohepta[c]pyridine-10-carboxylate 157-1 was synthesised from tert-butyl (±)-1-fluoro-9-methylene-6,7,8,9-tetrahydro-5H-5,8-epiminocyclohepta[c]pyridine-10-carboxylate 154-8 using a procedure essentially the same as for 155. LCMS (Method 5) m/z 293.3 (M+H)⁺ (ES)⁺ at 1.86 min. ¹H NMR (400 MHz, DMSO-d6) δ 8.44 (d, J=4.8 Hz, 1H), 7.59 (d, J=5.2 Hz, 1H), 5.23 (d, J=6.4 Hz, 1H), 4.51 (d, J=8.0 Hz, 1H), 2.45-2.36 (m, 2H), 1.81-1.76 (m, 2H), 1.25 (s, 9H)

Step 2: tert-Butyl 1-fluoro-9-hydroxy-6,7,8,9-tetrahydro-5H-5,8-epiminocyclohepta[c]pyridine-10-carboxylate 157-2 was synthesised from tert-butyl 1-fluoro-9-oxo-6,7,8,9-tetrahydro-5H-5,8-epiminocyclohepta[c]pyridine-10-carboxylate 157-1 using a procedure essentially the same as for 156. LCMS (Method 5) m/z 295.3 (M+H)⁺ (ES)⁺ at 1.66 min. ¹H NMR (400 MHz, DMSO-d6) δ 8.04 (d, J=5.2 Hz, 1H), 7.20 (d, J=6.4 Hz, 1H), 5.11 (d, J=5.6 Hz, 1H), 4.90 (d, J=6.4 Hz, 1H), 4.30 (t, J=14 Hz, 1H), 2.29-2.21 (m, 1H), 2.15-2.05 (m, 1H), 1.93-1.90 (m, 1H), 1.72-1.66 (m, 1H), 1.35 (s, 9H).

Step 3: To a solution of tert-butyl 1-fluoro-9-hydroxy-6,7,8,9-tetrahydro-5H-5,8-epiminocyclohepta[c]pyridine-10-carboxylate 157-2 (30 mg, 0.1 mmol) in DCM (5 ml) was added Deoxo-Fluor (44.2 mg, 0.2 mol, 2 eq) at 0° C. under N₂. The reaction mixture was allowed to warm to RT and stirred for 16 h. The reaction mixture was basified with saturated aqueous NaHCO₃ solution and extracted with DCM (3×10 ml). The combined organics were dried over Na₂SO₄ and concentrated in vacuo. The product was purified by prep-TLC (33% EtOAc/petroleum ether) to give tert-butyl 1,9-difluoro-6,7,8,9-tetrahydro-5H-5,8-epiminocyclohepta[c]pyridine-10-carboxylate 157-3 as a yellow oil. LCMS (Method 5) m/z 296.8 (M+H)⁺ (ES)⁺ at 1.51 min

Step 4: To a solution of tert-butyl 1,9-difluoro-6,7,8,9-tetrahydro-5H-5,8-epiminocyclohepta[c]pyridine-10-carboxylate 157-3 (30 mg, 0.1 mmol) in 1,4-dioxane (3 ml) was added a solution of HCl in 1,4-dioxane (3 ml, 4 M) under N₂. The reaction mixture was stirred at RT for 1 h. The reaction mixture was basified with saturated aqueous NaHCO₃ solution and the product was extracted with DCM (3×10 ml). The combined organics phases were dried over Na₂SO₄ and concentrated in vacuo to afford 1,9-difluoro-6,7,8,9-tetrahydro-5H-5,8-epiminocyclohepta[c]pyridine 157-4 as a yellow oil. LCMS (Method 5) m/z 196.4 (M+H)⁺ (ES)⁺ at 0.28 min

Step 5: (±)-(9R)—N-(5-Chloro-2-fluoro-4-(trifluoromethyl)phenyl)-1,9-difluoro-6,7,8,9-tetrahydro-5H-5,8-epiminocyclohepta[c]pyridine-10-carboxamide 157 was synthesised from 1,9-difluoro-6,7,8,9-tetrahydro-5H-5,8-epiminocyclohepta[c]pyridine 157-4 and 5-chloro-2-fluoro-4-(trifluoromethyl)aniline using a procedure essentially the same as for 154. LCMS (Method 3) m/z 434.0 (M+H)⁺ (ES)⁺ at 3.43 min. ¹H NMR (400 MHz, DMSO-d6) δ 9.14 (s, 1H), 8.27 (d, J=7.2 Hz, 1H), 8.02 (d, J=6.8 Hz, 1H), 7.80 (d, J=11.2 Hz, 1H), 7.40 (d, J=5.2 Hz, 1H), 5.60 (d, J=47.2 Hz, 1H), 5.36 (d, J=6.4 Hz, 1H), 5.25 (t, J=20 Hz, 1H), 2.23-1.98 (m, 3H), 1.54-1.45 (m, 1H).

### Experimental Scheme 17

### Compound 158: (±)—N-(4,5-Dichloro-2-fluorophenyl)-2-oxo-2,5,6,7,8,9-hexahydro-1H-5,8-epiminocyclohepta[b]pyrazine-10-carboxamide

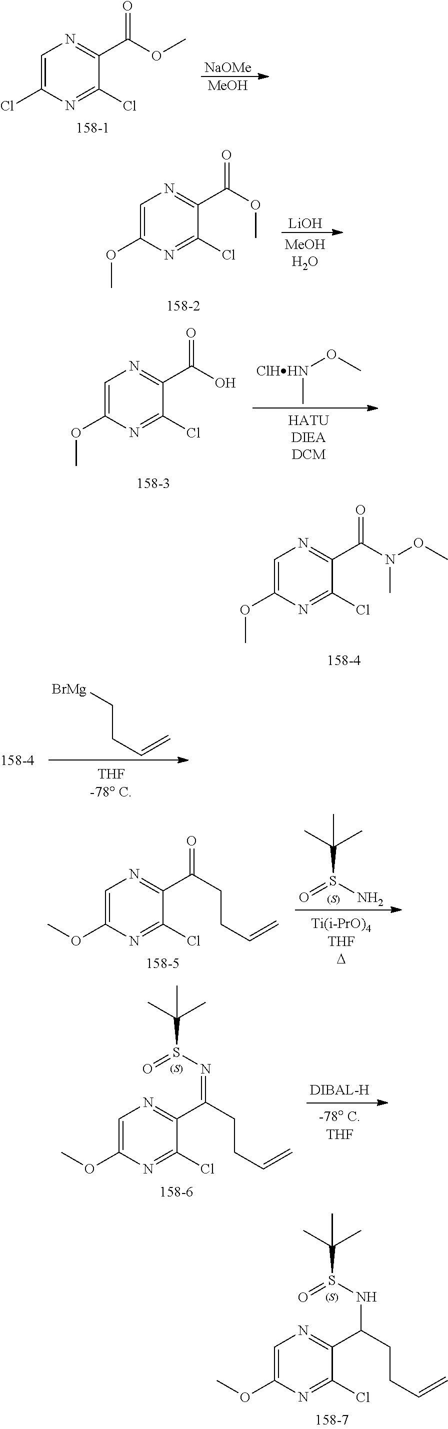

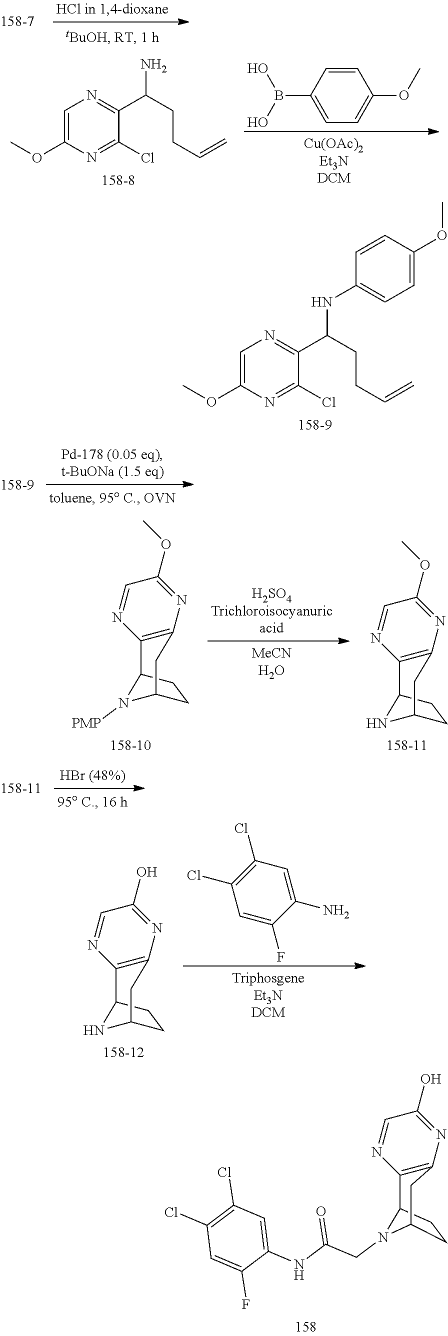

Step 1: To a solution of methyl 3,5-dichloropyrazine-2-carboxylate 158-1 (10.0 g, 48.54 mmol) in MeOH (100 ml) was added NaOMe (2.6 g, 48.54 mmol) slowly at 0° C. After addition, the reaction was stirred at RT for 1 h. The reaction was quenched by pouring onto ice water (300 ml) and the product was extracted with EtOAc (2×150 ml). The combined organics were dried with Na₂SO₄ and concentrated in vacuo to give methyl 3-chloro-5-methoxypyrazine-2-carboxylate 158-2 as a yellow solid. ¹H NMR (400 MHz, DMSO-d6) δ 8.40 (s, 1H), 3.99 (s, 3H), 3.88 (s, 3H)

Step 2: To a solution of methyl 3-chloro-5-methoxypyrazine-2-carboxylate 158-2 (9.5 g, 47.03 mmol) in MeOH/water (100/20 ml) was added LiOH (2.3 g, 94.06 mmol). The reaction was stirred at RT for 1 h. The mixture was acidified by addition of 2 M HCl to pH=7-8. The mixture was diluted with water (10 ml) and the product was extracted with DCM (2×100 ml). The combined organics were dried over Na₂SO₄ and concentrated in vacuo to give 3-chloro-5-methoxypyrazine-2-carboxylic acid 158-3 as a yellow solid. LCMS (Method 5) m/z 187.0 (M−H)⁻ (ES)⁻ at 0.99 min

Step 3: A mixture of 3-chloro-5-methoxypyrazine-2-carboxylic acid 158-3 (8.8 g, 46.81 mmol), N,O-dimethylhydroxylamine hydrochloride (4.5 g, 46.81 mmol), HATU (26.7 g, 70.21 mmol) and DIPEA (24.4 ml, 140.43 mmol) in DCM (100 ml) was stirred at RT for 16 h. The mixture was concentrated in vacuo and the product was purified by chromatography on silica gel (5% EtOAc/petroleum Ether) to give 3-chloro-N,5-dimethoxy-N-methylpyrazine-2-carboxamide 158-4 as a pale yellow solid. ¹H NMR (400 MHz, DMSO-d6) δ 8.38 (s, 1H), 3.97 (s, 3H), 3.51 (s, 3H), 3.31 (s, 3H)

Step 4: To a solution of 3-chloro-N,5-dimethoxy-N-methylpyrazine-2-carboxamide 158-4 (8.5 g, 36.80 mmol) in THF (100 ml) was added a solution of but-3-en-1-ylmagnesium bromide in THF (44.2 ml, 1 M, 44.16 mmol) dropwise at −78° C. under N₂. After addition, the reaction was stirred at −78° C. for 30 mins. The reaction was quenched with saturated aqueous NH₄Cl (50 ml) and the mixture was diluted with water (50 ml) before the product was extracted with EtOAc (2×100 ml). The combined organics were dried over Na₂SO₄ and concentrated in vacuo. The product was purified by chromatography on silica gel (5% EtOAc/petroleum ether) to give 1-(3-chloro-5-methoxypyrazin-2-yl) pent-4-en-1-one 158-5 as a yellow oil. ¹H NMR (400 MHz, DMSO-d6) δ 8.42 (s, 1H), 5.90-5.80 (m, 1H), 5.07-4.96 (m, 2H), 4.00 (s, 3H), 3.15 (t, J=7.2 Hz, 2H), 2.37-2.32 (m, 2H).

Step 5: A mixture of 1-(3-chloro-5-methoxypyrazin-2-yl) pent-4-en-1-one 158-5 (3.3 g, 14.60 mmol), (S)-2-methylpropane-2-sulfinamide (5.3 g, 43.80 mmol) and Ti(ⁱPrO)₄ (21.6 ml, 73.10 mmol) in THF (30 ml) was refluxed for 6 h under N₂. The reaction mixture was diluted with water (100 ml) and EtOAc (200 ml) then filtered over celite. The filtrate was separated and the organics were washed with brine (100 ml). The combined organics were dried over Na₂SO₄ and concentrated in vacuo. The product was purified by chromatography on silica gel (10% EtOAc/petroleum ether) to give (S)—N-(1-(3-chloro-5-methoxypyrazin-2-yl)pent-4-en-1-ylidene)-2-methylpropane-2-sulfinamide 158-6 as a yellow oil. ¹H NMR (400 MHz, DMSO-d6) δ 8.34 (s, 1H), 5.92-5.86 (m, 1H), 5.12-4.99 (m, 2H), 3.95 (s, 3H), 2.94-2.72 (m, 2H), 2.39-2.38 (m, 2H), 1.16 (s, 9H) Step 6: To a solution of (S)—N-(1-(3-chloro-5-methoxypyrazin-2-yl)pent-4-en-1-ylidene)-2-methylpropane-2-sulfinamide 158-6 (3 g, 9.12 mmol) in dry THF (30 ml) was added DIBAL (13.7 ml, 1 M, 13.7 mmol) dropwise at −78° C. under N₂. The reaction was stirred at −78° C. for 1 h. The reaction was poured onto water (10 ml) and MeOH (20 ml) and the mixture was extracted with DCM (2×30 ml). The combined organics were dried over Na₂SO₄ and concentrated in vacuo. The product was purified by chromatography on silica gel (10% EtOAc/petroleum ether) to give (S)—N-(1-(3-chloro-5-methoxypyrazin-2-yl)pent-4-en-1-yl)-2-methyl propane-2-sulfinamide 158-7 as a yellow oil. ¹H NMR (400 MHz, DMSO-d6) δ 8.37 (s, 1H), 5.82-5.74 (m, 1H), 5.54 (d, J=7.2 Hz, 0.7H), 5.39 (d, J=9.2 Hz, 0.3H), 5.03-4.96 (m, 2H), 4.72-4.61 (m, 1H), 3.92 (s, 3H), 2.12-1.98 (m, 3H), 1.94-1.73 (m, 1H), 1.10 (s, 3H), 1.02 (s, 6H).

Step 7: To a solution of (S)—N-(1-(3-chloro-5-methoxypyrazin-2-yl)pent-4-en-1-yl)-2-methylpropane-2-sulfinamide 158-7 (2.5 g, 7.55 mmol) in ^{t}BuOH (25 ml) was added HCl in 1,4-dioxane (9.4 ml, 4 M, 37.75 mmol). The reaction was stirred at RT for 1 h. The reaction mixture was basified by addition of 2 M NaHCO₃ to pH=7-8. The mixture was diluted with water (20 ml) before extraction with DCM (2×50 ml). The combined organics were dried over Na₂SO₄ and concentrated in vacuo to afford (±)-1-(3-chloro-5-methoxypyrazin-2-yl)pent-4-en-1-amine 158-8 as a yellow oil. ¹H NMR (400 MHz, DMSO-d6) δ 8.36 (s, 1H), 5.83-5.72 (m, 1H), 5.01-4.92 (m, 2H), 4.24 (t, J=6.5 Hz, 1H), 3.92 (s, 3H), 2.41-2.15 (m, 1H), 2.12-1.90 (m, 2H), 1.83-1.69 (m, 2H), 1.67-1.55 (m, 1H).

Step 8: A mixture of (±)-1-(3-chloro-5-methoxypyrazin-2-yl)pent-4-en-1-amine 158-8 (1.7 g, 7.49 mmol), (4-methoxyphenyl)boronic acid (4.8 g, 31.46 mmol), Cu(OAc)₂ (3.0 g, 14.98 mmol) and Et₃N (6.4 ml, 46.44 mmol) in DCM (30 ml) was stirred at RT for 16 h under O₂. The reaction mixture was concentrated in vacuo. The product was purified by chromatography on silica gel (10% EtOAc/petroleum ether) to give (±)—N-(1-(3-chloro-5-methoxypyrazin-2-yl)pent-4-en-1-yl)-4-methoxyaniline 158-9 as a yellow oil. ¹H NMR (400 MHz, DMSO-d6) δ 8.31 (s, 1H), 6.64-6.50 (m, 4H), 5.86-5.76 (m, 1H), 5.59 (d, J=9.6 Hz, 1H), 5.02-4.95 (m, 2H), 4.78-4.72 (m, 1H), 3.89 (s, 3H), 3.58 (s, 3H), 2.27-2.14 (m, 1H), 2.09-1.99 (m, 1H), 1.95-1.79 (m, 2H).

Step 9: To a solution of (±)—N-(1-(3-chloro-5-methoxypyrazin-2-yl)pent-4-en-1-yl)-4-methoxy aniline 158-9 (400 mg, 1.2 mmol) in dry toluene (5 ml) was added NaO^{t}Bu (173 mg, 1.8 mmol) and Pd-178 (29 mg, 0.06 mmol) in order under N₂. The reaction was stirred at 95° C. under N₂ for 6 h. The reaction mixture was concentrated in vacuo. The product was purified by chromatography on silica gel (20% EtOAc/petroleum ether) to give (±)-2-methoxy-10-(4-methoxyphenyl)-6,7,8,9-tetrahydro-5H-5,8-epiminocyclohepta[b]pyrazine 158-10 as a yellow solid. ¹H NMR (400 MHz, DMSO-d6) δ 7.98 (s, 1H), 6.77-6.70 (m, 4H), 4.76 (d, J=5.6 Hz, 1H), 4.55 (t, J=5.2 Hz, 1H), 3.78 (s, 3H), 3.61 (s, 3H), 3.19-3.13 (m, 1H), 2.53-2.48 (m, 1H), 2.36-2.23 (m, 2H), 1.89-1.85 (m, 1H), 1.79-1.73 (m, 1H).

Step 10: To a solution of (±)-2-methoxy-10-(4-methoxyphenyl)-6,7,8,9-tetrahydro-5H-5,8-epiminocyclohepta[b]pyrazine 158-10 (1.1 g, 3.70 mmol) in MeCN/water (10 ml/10 ml) was added H₂SO₄ (0.185 ml, 2 M, 3.70 mmol) and trichloroisocyanuric acid (429 mg, 1.85 mmol). The reaction was stirred at RT for 16 h. The reaction mixture was extracted with DCM (2×10 ml). The combined organics were extracted with water (10 ml). The aqueous layer was basified with NaOH (2 M) to pH˜ 10 and extracted with 10% MeOH in DCM (2×10 ml). The combined organics were dried over Na₂SO₄ and concentrated in vacuo to afford (±)-2-methoxy-6,7,8,9-tetrahydro-5H-5,8-epiminocyclohepta[b]pyrazine 158-11 as a brown oil. ¹H NMR (400 MHz, DMSO-d6) δ 7.91 (s, 1H), 4.12-4.11 (m, 1H), 3.83 (s, 3H), 3.77 (t, J=5.2 Hz, 1H), 3.09-3.03 (m, 1H), 2.90 (s, 1H), 2.51-2.47 (m, 1H), 2.00-1.88 (m, 2H), 1.77-1.73 (m, 1H), 1.50-1.45 (m, 1H).

Step 11: A mixture of (±)-2-methoxy-6,7,8,9-tetrahydro-5H-5,8-epiminocyclohepta[b]pyrazine 158-11 (600 mg, 3.14 mmol) and HBr (6 ml, 48% w/w in water) was stirred at 95° C. for 16 h. The mixture was concentrated in vacuo to give (±)-6,7,8,9-tetrahydro-5H-5,8-epiminocyclohepta[b]pyrazin-2-ol hydrobromide salt 158-12 as a brown solid. LCMS (Method 5) m/z 178.2 (M+H)⁺ (ES)⁺ at 0.47 min.

Step 12: To a solution of 4,5-dichloro-2-fluoroaniline (50 mg, 0.28 mmol) and triphosgene (41 mg, 0.14 mmol) in DCM (3 ml) was added a solution of DMAP (109 mg, 0.90 mmol) in DCM (1 ml) at 0° C. The resulting mixture was stirred at 0° C. for 30 min. This was added to a solution of (±)-6,7,8,9-tetrahydro-5H-5,8-epiminocyclohepta[b]pyrazin-2-ol hydrobromide salt 158-12 and Et₃N (0.1 ml, 0.56 mmol, 2 eq) in DCM (2 ml) The reaction was stirred at RT for 16 h. The reaction mixture was poured into water (5 ml) and extracted with 10% MeOH in DCM (2×10 ml). The combined organics were dried over Na₂SO₄ and concentrated in vacuo. The product was purified by prep-TLC (10% MeOH/DCM) to give (±)—N-(4,5-dichloro-2-fluorophenyl)-2-oxo-2,5,6,7,8,9-hexahydro-1H-5,8-epiminocyclohepta[b]pyrazine-10-carboxamide 158 as a brown solid. LCMS (Method 3) m/z 383.0 (M+H)⁺ (ES)⁺ at 2.59 min. ¹H NMR (400 MHz, DMSO-d6) δ 12.01 (s, 1H), 8.80 (s, 1H), 7.84 (d, J=7.6 Hz, 1H), 7.75 (s, 1H), 7.65 (d, J=10.4 Hz, 1H), 5.03 (d, J=5.6 Hz, 1H), 4.65 (t, J=6.4 Hz, 1H), 3.23-3.18 (m, 1H), 2.47-2.43 (m, 1H), 2.27-2.07 (m, 2H), 1.92-1.82 (m, 1H), 1.73-1.66 (m, 1H)

### Experimental Scheme 18

### Compound 159: (5R,8S)—N-(4,5-dichloro-2-fluorophenyl)-1-fluoro-4-(methylamino)-6,7,8,9-tetrahydro-5H-5,8-epiminocyclohepta[c]pyridine-10-carboxamide

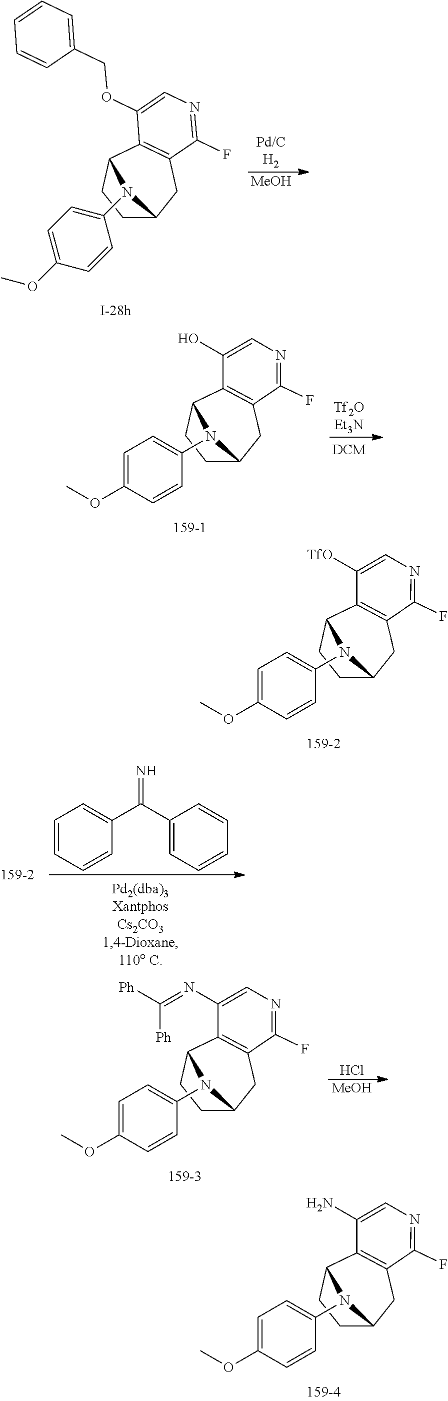

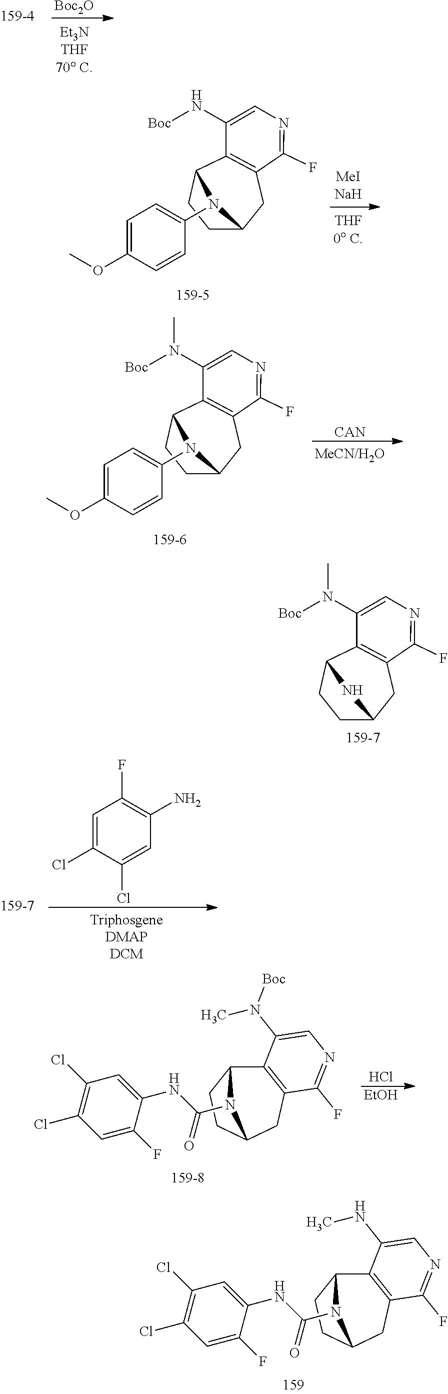

Step 1: To a solution of (5R,8S)-4-(benzyloxy)-1-fluoro-10-(4-methoxyphenyl)-6,7,8,9-tetrahydro-5H-5,8-epiminocyclohepta[c]pyridine I-28 h (900 mg, 2.3 mmol) in MeOH (200 ml) was added Pd/C (100 mg) at RT. The mixture was stirred under a H₂ balloon for 16 h. After completion, the reaction mixture was filtered, the filter cake was washed with MeOH (100 ml) and the filtrate was concentrated in vacuo to give (5R,8S)-1-fluoro-10-(4-methoxyphenyl)-6,7,8,9-tetrahydro-5H-5,8-epiminocyclohepta[c]pyridin-4-ol 159-1 as a white solid. LCMS (Method 5) m/z 300.7 (M+H)⁺ (ES)⁺ at 1.20 min.

Step 2: To a solution of (5R,8S)-1-fluoro-10-(4-methoxyphenyl)-6,7,8,9-tetrahydro-5H-5,8-epiminocyclohepta[c]pyridin-4-ol 159-1 (690 mg, 2.3 mmol, 1.0 eq) and Et₃N (1.6 ml, 11.5 mmol) in DCM (80 ml) was added Tf₂O (1.95 g, 6.9 mmol) at 0° C. under N₂. The mixture was stirred at 0° C. for 3 h. After completion, the mixture was diluted with water (50 ml) and the product was extracted with DCM (3×30 ml). The combined organics were washed with brine (50 ml), dried over Na₂SO₄ and concentrated in vacuo. The product was purified by chromatography on silica gel column (20% EtOAc/petroleum ether) to give (5R,8S)-1-fluoro-10-(4-methoxyphenyl)-6,7,8,9-tetrahydro-5H-5,8-epiminocyclohepta[c]pyridin-4-yl trifluoromethanesulfonate 159-2 as a yellow solid. LCMS (Method 5) m/z 432.8 (M+H)⁺ (ES)⁺ at 2.08 min

Step 3: A mixture of (5R,8S)-1-fluoro-10-(4-methoxyphenyl)-6,7,8,9-tetrahydro-5H-5,8-epiminocyclohepta[c]pyridin-4-yl trifluoromethanesulfonate 159-2 (200 mg, 0.46 mmol), diphenylmethanimine (83.4 mg, 0.46 mmol), Pd₂(dba)₃ (47.6 mg, 0.046 mmol), xantphos (53.2 mg, 0.092 mmol) and Cs₂CO₃ (449.6 mg, 1.38 mmol) in dry 1,4-dioxane (2.5 ml) was stirred at 110° C. under N₂ for 16 h. The reaction mixture was diluted with water (20 ml) and extracted with EtOAc (3×20 ml). The combined organics were washed with brine (20 ml), dried over Na₂SO₄ and concentrated in vacuo. The product was purified by prep-TLC (25% EtOAc/petroleum ether) to give N-((5R,8S)-1-fluoro-10-(4-methoxyphenyl)-6,7,8,9-tetrahydro-5H-5,8-epiminocyclohepta[c]pyridin-4-yl)-1,1-diphenylmethanimine) 159-3 as a yellow oil. LCMS (Method 5) m/z 463.9 (M+H)⁺ (ES)⁺ at 2.36 min

Step 4: To a solution of N-((5R,8S)-1-fluoro-10-(4-methoxyphenyl)-6,7,8,9-tetrahydro-5H-5,8-epiminocyclohepta[c]pyridin-4-yl)-1,1-diphenylmethanimine 159-3 (200 mg, 0.43 mmol) in MeOH (8 ml) was added a solution of aqueous HCl (4 ml, 1 M) at RT under N₂. After stirring for 2 h, the reaction mixture was basified with saturated aqueous NaHCO₃ solution and the product was extracted with DCM (3×20 ml). The combined organics were dried over Na₂SO₄ and concentrated in vacuo. The product was purified by prep-TLC (50% EtOAc/petroleum ether) to give (5R,8S)-1-fluoro-10-(4-methoxyphenyl)-6,7,8,9-tetrahydro-5H-5,8-epiminocyclohepta[c]pyridin-4-amine 159-4 as a white solid. LCMS (Method 5) m/z 299.7 (M+H)⁺ (ES)⁺ at 1.11 min

Step 5: To a solution of (5R,8S)-1-fluoro-10-(4-methoxyphenyl)-6,7,8,9-tetrahydro-5H-5,8-epiminocyclohepta[c]pyridin-4-amine 159-4 (110 mg, 0.37 mmol) and Et₃N (103 μl, 0.74 mmol) in THF (8 ml) was added di-tert-butyl dicarbonate (122 mg, 0.56 mmol). The reaction mixture was stirred at 70° C. under N₂ for 16 h. The mixture was concentrated in vacuo and the product was purified by prep-TLC (33% EtOAc/petroleum ether) to give tert-butyl ((5R,8S)-1-fluoro-10-(4-methoxyphenyl)-6,7,8,9-tetrahydro-5H-5,8-epiminocyclohepta[c]pyridin-4-yl)carbamate 159-5 as a colourless oil. LCMS (Method 5) m/z 399.9 (M+H)⁺ (ES)⁺ at 1.72 min

Step 6: To a solution of tert-butyl ((5R,8S)-1-fluoro-10-(4-methoxyphenyl)-6,7,8,9-tetrahydro-5H-5,8-epiminocyclohepta[c]pyridin-4-yl)carbamate 159-5 (105 mg, 0.26 mmol) and NaH (9.4 mg, 60% w/w, 0.39 mmol) in THF (7 ml) was added methyl iodide (74 mg, 0.52 mmol) at 0° C. under N₂. After stirring for 2 h, the reaction mixture was diluted with water (20 ml) and extracted with EtOAc (3×20 ml). The combined organics were washed with brine, dried over Na₂SO₄ and concentrated in vacuo. The product was purified by prep-TLC (25% EtOAc/petroleum ether) to give tert-butyl ((5R,8S)-1-fluoro-10-(4-methoxyphenyl)-6,7,8,9-tetrahydro-5H-5,8-epiminocyclohepta[c]pyridin-4-yl)(methyl)carbamate 159-6 as a colorless oil. LCMS (Method 5) m/z 414.3 (M+H)⁺ (ES)⁺ at 2.47 min.

Step 7: To a solution of tert-butyl ((5R,8S)-1-fluoro-10-(4-methoxyphenyl)-6,7,8,9-tetrahydro-5H-5,8-epiminocyclohepta[c]pyridin-4-yl)(methyl)carbamate 159-6 (90 mg, 0.22 mmol) in MeCN (6 ml) was added, dropwise, a solution of CAN (361.8 mg, 0.66 mmol) in water (3 ml) at 0° C. under N₂. The reaction mixture was stirred at 0° C. for 30 min before warming to RT and stirred for another 1.5 h. 2 M NaOH aqueous solution (20 ml) and DCM (20 ml) was added into the reaction mixture. The mixture was filtered through a celite pad and the filter cake was washed with DCM (50 ml). The layers were separated and the aqueous layer was extracted with DCM (3×10 ml). The combined organics were washed with brine, dried over Na₂SO₄ and concentrated in vacuo to give tert-butyl ((5R,8S)-1-fluoro-6,7,8,9-tetrahydro-5H-5,8-epiminocyclohepta[c]pyridin-4-yl)(methyl)carbamate 159-7 as a red oil. LCMS (Method 5) m/z 308.2 (M+H)⁺ (ES)⁺ at 0.51 min.

Step 8: To a solution of 4,5-dichloro-2-fluoroaniline (40 mg, 0.22 mmol) and triphosgene (33 mg, 0.11 mmol) in DCM (3 ml) at 0° C. was added a solution of DMAP (86 mg, 0.70 mol) in DCM (1 ml). The mixture was stirred at 0° C. for 30 min. The reaction mixture was added to a solution of tert-butyl ((5R,8S)-1-fluoro-6,7,8,9-tetrahydro-5H-5,8-epiminocyclohepta[c]pyridin-4-yl)(methyl)carbamate 159-7 (67.6 mg, 0.22 mmol) in DCM (2 ml). The reaction mixture was stirred at RT for 16 h. After completion, the reaction mixture was poured into water (20 ml) and the product was extracted with DCM (3×10 ml). The combined organics were washed with brine, dried over Na₂SO₄ and concentrated in vacuo. The product was purified by prep-TLC (33% EtOAc/petroleum ether) to give tert-butyl ((5R,8S)-10-((4,5-dichloro-2-fluorophenyl)carbamoyl)-1-fluoro-6,7,8,9-tetrahydro-5H-5,8-epiminocyclohepta[c]pyridin-4-yl)(methyl)carbamate) 159-8 as a colorless oil. LCMS (Method 5) m/z 512.9 (M+H)⁺ (ES)⁺ at 2.13 min

Step 9: A mixture of tert-butyl ((5R,8S)-10-((4,5-dichloro-2-fluorophenyl)carbamoyl)-1-fluoro-6,7,8,9-tetrahydro-5H-5,8-epiminocyclohepta[c]pyridin-4-yl)(methyl)carbamate 159-8 (80 mg, 0.16 mol) in a solution of HCl in EtOH (5 ml, 4 M) was stirred at RT for 2 h. The reaction mixture was basified with saturated NaHCO₃ aqueous solution and extracted with DCM (3×20 ml). The combined organics were dried over Na₂SO₄ and concentrated in vacuo. The residue was purified by prep-TLC (5% MeOH/DCM) to give (5R,8S)—N-(4,5-dichloro-2-fluorophenyl)-1-fluoro-4-(methylamino)-6,7,8,9-tetrahydro-5H-5,8-epiminocyclohepta[c]pyridine-10-carboxamide 159 as a yellow solid. LCMS (Method 3) m/z 413.1 (M+H)⁺ (ES)⁺ at 3.42 min. ¹H NMR (400 MHz, DMSO-d6) δ 8.71 (s, 1H), 7.82-7.79 (m, 1H), 7.67 (d, J=10.4 Hz, 1H), 7.18 (s, 1H), 5.24 (d, J=6.4 Hz, 1H), 4.76-4.73 (m, 1H), 3.14 (dd, J=5.2, 4.8 Hz, 1H), 2.73 (s, 3H), 2.45 (d, J=17.2 Hz, 1H), 2.33-2.22 (m, 1H), 2.09-2.06 (m, 1H), 1.76-1.65 (m, 2H).

### Experimental Scheme 20

### Compound 160: (5R,8S)-4-Cyano-N-(4,5-dichloro-2-fluorophenyl)-1-fluoro-6,7,8,9-tetrahydro-5H-5,8-epiminocyclohepta[c]pyridine-10-carboxamide

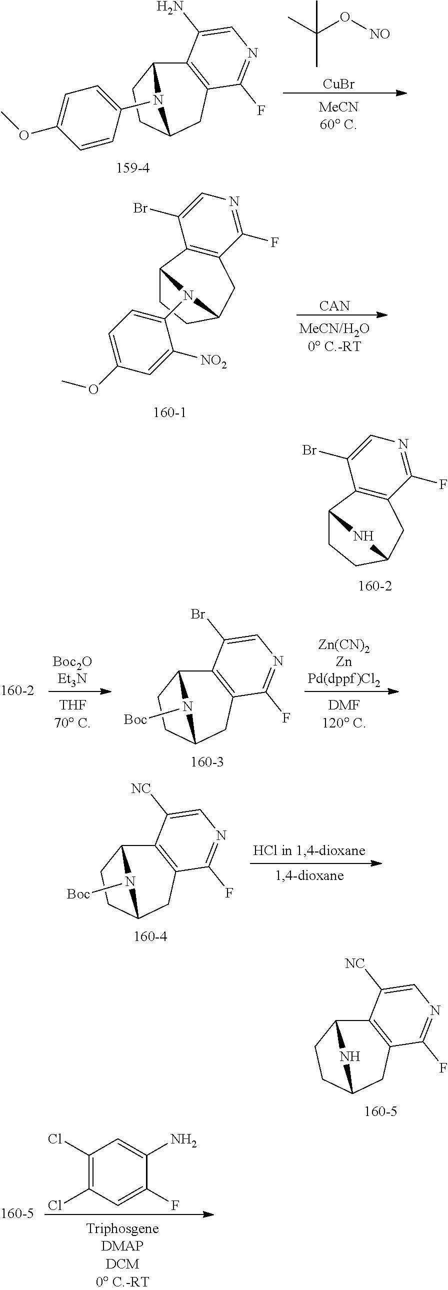

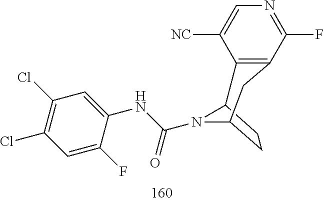 [SMILES: N#Cc1cnc(F)c2c1C1CCC(C2)N1C(=O)Nc1cc(Cl)c(Cl)cc1F]

Step 1: A mixture of (5R,8S)-1-fluoro-10-(4-methoxyphenyl)-6,7,8,9-tetrahydro-5H-5,8-epiminocyclohepta[c]pyridin-4-amine 159-4 (200 mg, 0.67 mmol), CuBr (192.2 mg, 1.34 mmol) and tert-butyl nitrite (138.2 mg, 1.34 mmol) in MeCN (8 ml) was stirred at 60° C. under N₂ for 16 h. The reaction mixture was diluted with water (20 ml) and the product was extracted with EtOAc (3×20 ml). The combined organics were washed with brine, dried with Na₂SO₄ and concentrated in vacuo. The product was purified by chromatography on silica gel column (20% EtOAc/petroleum ether) to give (5R,8S)-4-bromo-1-fluoro-10-(4-methoxy-2-nitrophenyl)-6,7,8,9-tetrahydro-5H-5,8-epiminocyclohepta[c]pyridine 160-1 as an orange solid. LCMS (Method 5) m/z 408, 410 (M+H)⁺ (ES)⁺ at 2.55 min.

Step 2: To a solution of (5R,8S)-4-bromo-1-fluoro-10-(4-methoxy-2-nitrophenyl)-6,7,8,9-tetrahydro-5H-5,8-epiminocyclohepta[c]pyridine 160-1 (150 mg, 0.37 mmol) in MeCN (8 ml) was added dropwise a solution of CAN (608.5 mg, 1.11 mmol) in water (4 ml) at 0° C. under N₂. The reaction mixture was stirred at 0° C. for 30 min before warming to RT and stirred for another 16 h. 2 M NaOH aqueous solution (40 ml) and DCM (50 ml) was added to the reaction mixture. The mixture was filtered through a celite pad and the filter cake was washed with DCM (100 ml). The filtrate was separated and the aqueous layer was extracted with DCM (3×20 ml). The combined organics were washed with brine (100 ml), dried with Na₂SO₄ and concentrated in vacuo. The product was purified by prep-TLC (10% MeOH/DCM) to give (5R,8S)-4-bromo-1-fluoro-6,7,8,9-tetrahydro-5H-5,8-epiminocyclohepta[c]pyridine 160-2 as a yellow oil. LCMS (Method 5) m/z 257, 259 (M+H)⁺ (ES)⁺ at 0.58 min.

Step 3: To a solution of (5R,8S)-4-bromo-1-fluoro-6,7,8,9-tetrahydro-5H-5,8-epiminocyclohepta[c]pyridine 160-2 (39 mg, 0.15 mmol) and Et₃N (38 μl, 0.3 mmol) in THF (5 ml) was added di-tert-butyl dicarbonate (50 mg, 0.23 mmol). The reaction mixture was stirred at 70° C. under N₂ for 16 h. The mixture was concentrated in vacuo and the residue was purified by prep-TLC (10% EtOAc/petroleum ether) to give tert-butyl (5R,8S)-4-bromo-1-fluoro-6,7,8,9-tetrahydro-5H-5,8-epiminocyclohepta[c]pyridine-10-carboxylate 160-3 as a white solid. LCMS (Method 5) m/z 357, 359 (M+H)⁺ (ES)⁺ at 2.54 min.

Step 4: A mixture of tert-butyl (5R,8S)-4-bromo-1-fluoro-6,7,8,9-tetrahydro-5H-5,8-epiminocyclohepta[c]pyridine-10-carboxylate 160-3 (53 mg, 0.15 mmol), Zn(CN)₂ (10.5 mg, 0.09 mmol), Zn (1 mg, 0.015 mmol) and Pd(dppf)Cl₂ (11 mg, 0.015 mmol) in dry DMF (2 ml) was stirred at 120° C. under N₂ for 16 h. The reaction mixture was diluted with water (20 ml) and the product was extracted with EtOAc (3×10 ml). The combined organics were washed with brine, dried over Na₂SO₄ and concentrated in vacuo. The product was purified by prep-TLC (25% EtOAc/petroleum ether) to give tert-butyl (5R,8S)-4-cyano-1-fluoro-6,7,8,9-tetrahydro-5H-5,8-epiminocyclohepta[c]pyridine-10-carboxylate 160-4 as a white solid. ¹H NMR (400 MHz, DMSO-d6) δ 8.64 (s, 1H), 5.03 (d, J=6.0 Hz, 1H), 4.49-4.46 (m, 1H), 3.11 (dd, J=4.4, 4.8 Hz 1H), 2.64 (d, J=18 Hz, 1H), 2.32-2.22 (m, 1H), 1.93-1.88 (m, 1H), 1.76-1.70 (m, 1H), 1.32 (s, 9H).

Step 5: To a solution of tert-butyl (5R,8S)-4-cyano-1-fluoro-6,7,8,9-tetrahydro-5H-5,8-epiminocyclohepta[c]pyridine-10-carboxylate 160-4 (25 mg, 0.08 mmol) in 1,4-dioxane (3 ml) was added a solution of HCl in 1,4-dioxane (1.5 ml, 4 M) at RT. After stirring for 3 h, the reaction mixture was basified with saturated NaHCO₃ aqueous solution and extracted with DCM (3×10 ml). The combined organics were dried over Na₂SO₄ and concentrated in vacuo to give (5R,8S)-1-fluoro-6,7,8,9-tetrahydro-5H-5,8-epiminocyclohepta[c]pyridine-4-carbonitrile 160-5 as a yellow oil. LCMS (Method 5) m/z 204.2 (M+H)⁺ (ES)⁺ at 0.45 min.

Step 6: To a solution of 4,5-dichloro-2-fluoroaniline (14 mg, 0.08 mmol) and triphosgene (12 mg, 0.04 mmol) in DCM (3 ml) at 0° C. was added a solution of DMAP (32 mg, 0.26 mol) in DCM (1 ml). The mixture was stirred at 0° C. for 30 min. This mixture was added to a solution of (5R,8S)-1-fluoro-6,7,8,9-tetrahydro-5H-5,8-epiminocyclohepta[c]pyridine-4-carbonitrile 160-5 (16 mg, 0.08 mol) in DCM (2 ml). The reaction mixture was stirred at RT for 16 h. The reaction was poured into water and extracted with DCM (3×10 ml). The combined organics were washed with brine (20 ml), dried over Na₂SO₄ and concentrated in vacuo. The product was purified by prep-TLC (33% EtOAc/petroleum ether) to give (5R,8S)-4-cyano-N-(4,5-dichloro-2-fluorophenyl)-1-fluoro-6,7,8,9-tetrahydro-5H-5,8-epiminocyclohepta[c]pyridine-10-carboxamide 160 as a white solid. LCMS (Method 3) m/z 407.0 (M+H)⁺ (ES)⁺ at 3.47 min. ¹H NMR (400 MHz, DMSO-d6) δ 9.06 (s, 1H), 8.63 (s, 1H), 7.84 (d, J=7.6 Hz, 1H), 7.68 (d, J=10.4 Hz, 1H), 5.31 (d, J=5.6 Hz, 1H), 4.83-4.80 (m, 1H), 3.25 (dd, J=5.2, 4.8 Hz, 1H), 2.66 (d, J=18 Hz, 1H), 2.32-2.27 (m, 2H), 1.98-1.92 (m, 1H), 1.82-1.78 (m, 1H).

### Experimental Scheme 21

### Compound 161: (5R,8S)—N-(5-Chloro-2-fluoro-4-(trifluoromethyl)phenyl)-1-fluoro-4-(3-(methylamino)propoxy)-6,7,8,9-tetrahydro-5H-5,8-epiminocyclohepta[c]pyridine-10-carboxamide

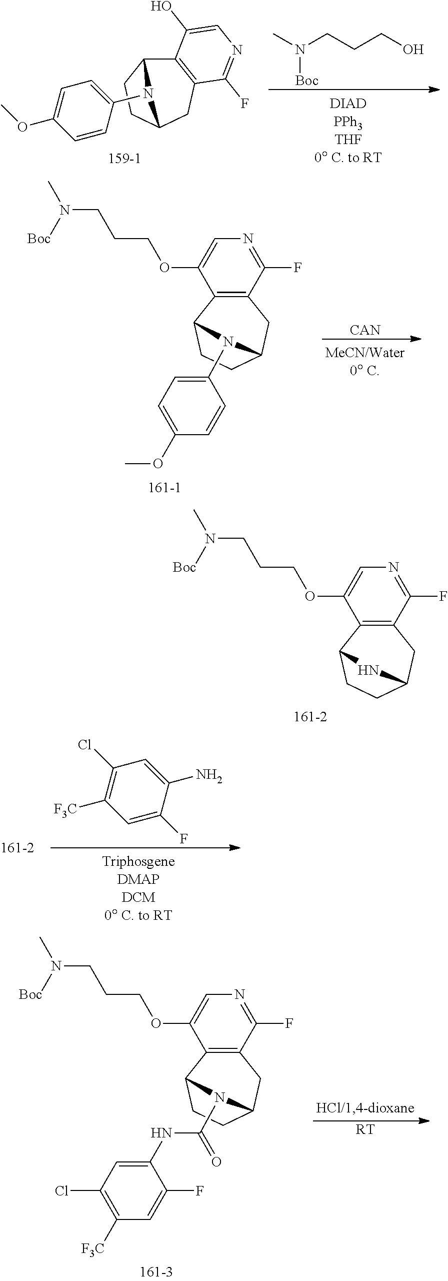

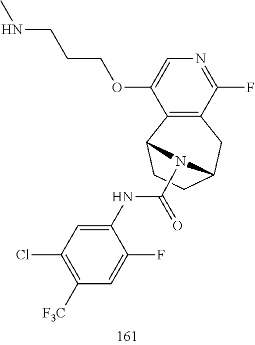 [SMILES: CNCCCOc1cnc(F)c2c1[C@H]1CC[C@@H](C2)N1C(=O)Nc1cc(Cl)c(C(F)(F)F)cc1F]

Step 1: To a solution of methyl (5R,8S)-1-fluoro-10-(4-methoxyphenyl)-6,7,8,9-tetrahydro-5H-5,8-epiminocyclohepta[c]pyridin-4-ol 159-1 (100 mg, 0.33 mmol), tert-butyl (3-hydroxypropyl)(methyl)carbamate (125 mg, 0.66 mmol) and PPh₃ (173 mg, 0.66 mmol) in THF (5 ml) at 0° C. was added DIAD (133 mg, 0.66 mmol) dropwise under N₂. The reaction mixture was warmed up to RT and stirred for 16 h. The reaction mixture was poured into water (20 ml) and the product was extracted with EtOAc (2×20 ml). The combined organics were washed with brine, dried with Na₂SO₄ and concentrated in vacuo. The product was purified by prep-TLC (25% EtOAc/petroleum ether) to give tert-butyl (3-(((5R,8S)-1-fluoro-10-(4-methoxyphenyl)-6,7,8,9-tetrahydro-5H-5,8-epiminocyclohepta[c]pyridin-4-yl)oxy)propyl)(methyl)carbamate 161-1 as a white solid. LCMS (Method 5) m/z 472.1 (M+H)⁺ (ES)⁺ at 1.81 min.

Step 2: To a solution of tert-butyl (3-(((5R,8S)-1-fluoro-10-(4-methoxyphenyl)-6,7,8,9-tetrahydro-5H-5,8-epiminocyclohepta[c]pyridin-4-yl)oxy)propyl)(methyl)carbamate 161-1 (145 mg, 0.31 mmol) in MeCN (4 ml) at 0° C. was added a solution of CAN (510 mg, 0.93 mmol) in water (3 ml). After stirring at RT for 2 h, the mixture was poured into aqueous NaOH solution (3 ml, 2 M) and filtered through celite, the filter cake was washed with DCM (20 ml). The filtrate was separated and the aqueous layer was extracted with DCM (20 ml). The combined organics were washed with brine, dried over Na₂SO₄ and concentrated in vacuo to give tert-butyl (3-(((5R,8S)-1-fluoro-6,7,8,9-tetrahydro-5H-5,8-epimino cyclohepta[c]pyridin-4-yl)oxy)propyl)(methyl)carbamates 161-2 a yellow solid. LCMS (Method 3) m/z 366.2 (M+H)⁺ (ES)⁺ at 1.07 min.

Step 3: To a solution of 5-chloro-2-fluoro-4-(trifluoromethyl)aniline (66 mg, 0.31 mmol) and triphosgene (45 mg, 0.15 mmol) in dry DCM (1 ml) at 0° C. was slowly added a solution of DMAP (120 mg, 0.99 mmol) in dry DCM (1 ml). After stirring at 0° C. for 30 mins, tert-butyl (3-(((5R,8S)-1-fluoro-6,7,8,9-tetrahydro-5H-5,8-epiminocyclohepta[c]pyridin-4-yl)oxy)propyl)(methyl)carbamate 161-2 (112 mg, 0.31 mmol) was added. The reaction solution was stirred at RT for 16 h. The reaction mixture was poured into water (20 ml) and the product was extracted with DCM (2×20 ml). The combined organics were washed with brine (20 ml), dried over Na₂SO₄ and concentrated in vacuo. The product was purified by prep-TLC (25% EtOAc/petroleum ether) to give tert-butyl (3-(((5R,8S)-10-((5-chloro-2-fluoro-4-(trifluoromethyl)phenyl) carbamoyl)-1-fluoro-6,7,8,9-tetrahydro-5H-5,8-epiminocyclohepta[c]pyridin-4-y)oxy)propyl)(methyl)carbamate 161-3 as a white solid. LCMS (Method 5) m/z 505.3 (M-Boc+H)⁺ (ES)⁺ at 2.06 min

Step 4: To a solution of tert-butyl (3-(((5R,8S)-10-((5-chloro-2-fluoro-4-(trifluoromethyl)phenyl)carbamoyl)-1-fluoro-6,7,8,9-tetrahydro-5H-5,8-epiminocyclohepta[c]pyridin-4-yl)oxy)propyl)(methyl)carbamate 161-3 (57 mg, 0.09 mmol) in 1,4-dioxane (2 ml) was added a solution of HCl in 1,4-dioxane (3 ml, 4 M) at 0° C. The reaction mixture was stirred at RT for 1 h. The reaction mixture was concentrated, the residue was basified with saturated aqueous NaHCO₃ to pH=8-9 and extracted with DCM (3×20 ml). The combined organics were washed with brine, dried over Na₂SO₄ and concentrated in vacuo. The product was purified by prep-TLC (10% MeOH/DCM) to give (5R,8S)—N-(5-chloro-2-fluoro-4-(trifluoromethyl)phenyl)-1-fluoro-4-(3-(methylamino)propoxy)-6,7,8,9-tetrahydro-5H-5,8-epiminocyclohepta[c]pyridine-10-carboxamide 161 as a white solid. LCMS (Method 5) m/z 505.2 (M+H)⁺ (ES)⁺ at 2.56 min. ¹H NMR (400 MHz, DMSO-d6) δ 8.04 (d, J=6.8 Hz, 1H), 7.78-7.73 (m, 2H), 5.44 (d, J=6.4 Hz, 1H), 4.81 (t, J=6.4 Hz, 1H), 4.19 (t, J=6.0 Hz, 2H), 3.21-3.15 (m, 1H), 2.96 (s, 3H), 2.61-2.52 (m, 1H), 2.32-1.69 (m, 8H).

### Experimental Scheme 22

### Compound 183: (±)—N-(5-Chloro-2-fluoro-4-(trifluoromethyl)phenyl)-1,9,9-trifluoro-6,7,8,9-tetrahydro-5H-5,8-epiminocyclohepta[c]pyridine-10-carboxamide

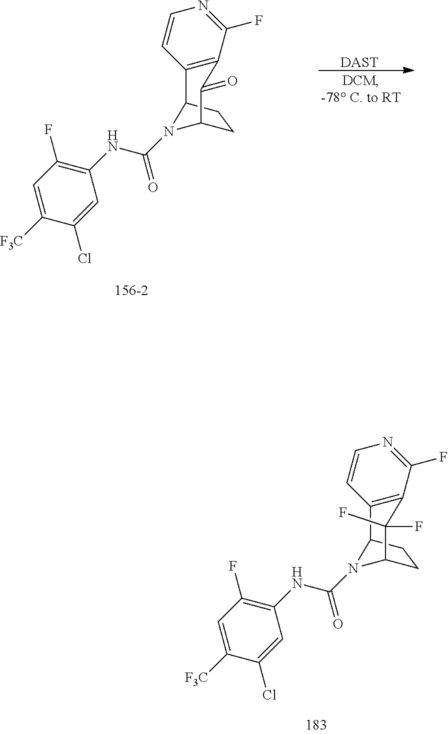 [SMILES: O=C1c2c(ccnc2F)C2CCC1N2C(=O)Nc1cc(Cl)c(C(F)(F)F)cc1F ; "DAST" ; O=C(Nc1cc(Cl)c(C(F)(F)F)cc1F)N1C2CCC1C(F)(F)c1c2ccnc1F]

Step 1: To a solution of (±)—N-(5-chloro-2-fluoro-4-(trifluoromethyl)phenyl)-1-fluoro-9-oxo-6,7,8,9-tetrahydro-5H-5,8-epiminocyclohepta[c]pyridine-10-carboxamide 156-2 (50 mg, 0.12 mmol) in DCM (5 ml) was added DAST (116 mg, 0.72 mol) at −78° C. under N₂. The reaction mixture was allowed to warm to RT and stirred for 16 h. The mixture was basified with NaHCO₃ aqueous solution and extracted with DCM (3×20 ml). The combined organics were dried over Na₂SO₄ and concentrated in vacuo. The residue was purified by prep-TLC (33% EtOAc/petroleum ether) to give (±)—N-(5-chloro-2-fluoro-4-(trifluoromethyl)phenyl)-1,9,9-trifluoro-6,7,8,9-tetrahydro-5H-5,8-epiminocyclohepta[c]pyridine-10-carboxamide as a white solid. LCMS (Method 3) m/z 452.0 (M−H)⁻ (ES)⁻ at 3.61 min. ¹H NMR (400 MHz, DMSO-d6) δ 9.40 (s, 1H), 8.38 (d, J=5.2 Hz, 1H), 7.99 (d, J=6.8 Hz, 1H), 7.83 (d, J=10.8 Hz, 1H), 7.51 (d, J=4.8 Hz, 1H), 5.47 (d, J=6.4 Hz, 1H), 5.28-5.23 (m, 1H), 2.35-2.29 (m, 1H), 2.24-2.17 (m, 1H), 2.00-1.89 (m, 1H), 1.75-1.69 (m, 1H).

### Experimental Scheme 23

### Compound 186: (6S,9R)—N-(benzo[c][1,2,5]thiadiazol-5-yl)-3-oxo-3,5,6,7,8,9-hexahydro-2H-6,9-epiminocyclohepta[c]pyridazine-10-carboxamide

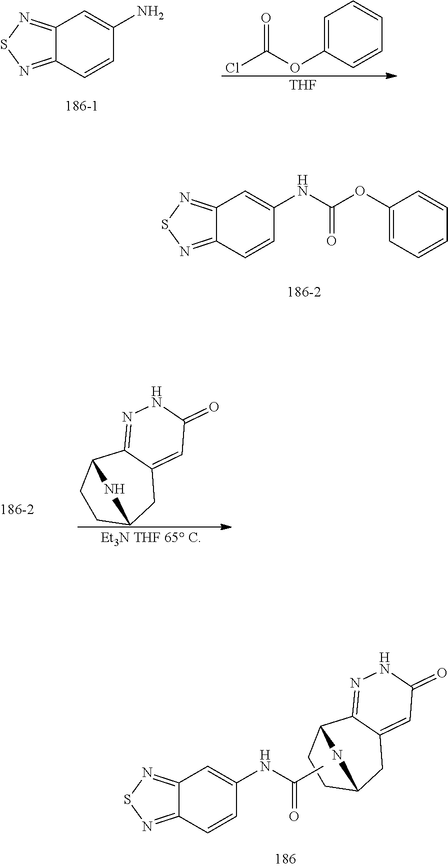

Step 1: To a solution of benzo[c][1,2,5]thiadiazol-5-amine 186-1 (101 mg, 668 μmol) in THF (1.00 ml) was added phenyl chloroformate (83.8 μl, 668 μmol) dropwise. The mixture was heated to 40° C. for 6 h. The reaction mixture was cooled to RT and used as a solution in the next step without any further purification.

Step 2: To a solution of (6S,9R)-2,5,6,7,8,9-hexahydro-3H-6,9-epiminocyclohepta[c]pyridazin-3-one (30.0 mg, 169 μmol) in THF (1 ml) was added a solution of phenyl benzo[c][1,2,5]thiadiazol-5-ylcarbamate 168-2 in THF (803 μl, 232 mM, 186 μmol) and the reaction was heated to 65° C. for 2 h. Et₃N (71 μl, 508 μmol) was added and heating continued for 16 h. The reaction was cooled to RT and concentrated in vacuo directly onto silica. The product was purified by chromatography on silica gel (0-10% MeOH/DCM) to afford (6S,9R)—N-(benzo[c][1,2,5]thiadiazol-5-yl)-3-oxo-3,5,6,7,8,9-hexahydro-2H-6,9-epiminocyclohepta[c]pyridazine-10-carboxamide 186 as a bright yellow powder. LCMS (Method 1) m/z 355.3 (M+H)⁺ (ES)⁺ at 0.76 min. ¹H NMR (400 MHz, DMSO-d6) δ 12.74 (s, 1H), 9.22 (s, 1H), 8.28 (d, J=2.0 Hz, 1H), 7.96 (d, J=9.5 Hz, 1H), 7.81 (dd, J=9.5, 2.1 Hz, 1H), 6.69 (s, 1H), 5.19 (d, J=5.7 Hz, 1H), 4.73 (t, J=6.2 Hz, 1H), 3.19 (dd, J=18.5, 5.2 Hz, 1H), 2.68 (d, J=18.2 Hz, 1H), 2.34-2.13 (m, 2H), 1.96-1.76 (m, 1H), 1.79-1.61 (m, 1H).

The following compounds were prepared using appropriate starting materials in an analogous procedure to that described in Experimental Scheme 23. Where the starting materials are not described in the literature, their synthesis is described below

| | | Method | |
|---|---|---|---|
| | | LCMS Rt | |
| Compound | Structure | [M + H]⁺ | NMR |
| 187^{(a)} | 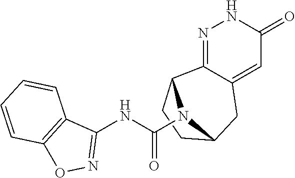 [SMILES: O=C(Nc1noc2ccccc12)N1[C@H]2CC[C@@H]1c1n[nH]c(=O)cc1C2] | Method 1 338.3 at 0.7 min | 1H NMR (400 MHz, DMSO-d6) δ 12.75 (s, 1H), 10.18 (s, 1H), 7.76 (dt, J = 8.0, 1.1 Hz, 1H), 7.68- 7.53 (m, 2H), 7.29 (ddd, J = 8.0, 6.6, 1.3 Hz, 1H), 6.72 (s, 1H), 5.19 (d, J = 6.3 Hz, 1H), 4.72 (t, J = 5.9 Hz, 1H), 3.25 (dt, J = 18.2, 3.3 Hz, 1H), 2.69 (d, J = 18.1 Hz, 1H), |
| | | | 2.34-2.17 (m, 2H), 1.91- |
| | (6S,9R)-N-(Benzo[d]isoxazol-3- | | 1.74 (m, 1H), 1.78-1.58 |
| | yl)-3-oxo-3,5,6,7,8,9-hexahydro- | | (m, 1H). |
| | 2H-6,9- | | |
| | epiminocyclohepta[c]pyridazine- | | |
| | 10-carboxamide | | |

^{(a)}compound purified by mass directed reverse phase HPLC

### Intermediate 73 (I-73)

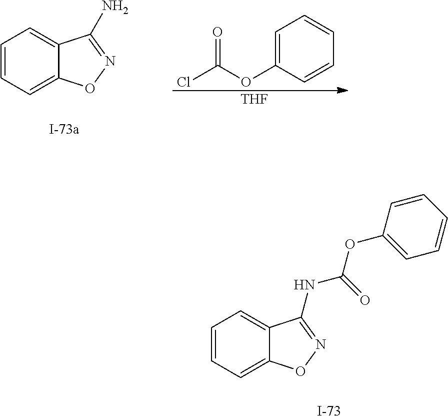

Phenyl benzo[d]isoxazol-3-ylcarbamate I-73 was synthesised from benzo[d]isoxazole-3-amine I-73a using a procedure essentially the same as for the synthesis of 168-2.

### Experimental Scheme 24

### Compound 204: (±)—(R)—N-(5-chloro-2-fluoro-4-(trifluoromethyl)phenyl)-1-fluoro-9-hydroxy-6,7,8,9-tetrahydro-5H-5,8-epiminocyclohepta[c]pyridine-10-carboxamide

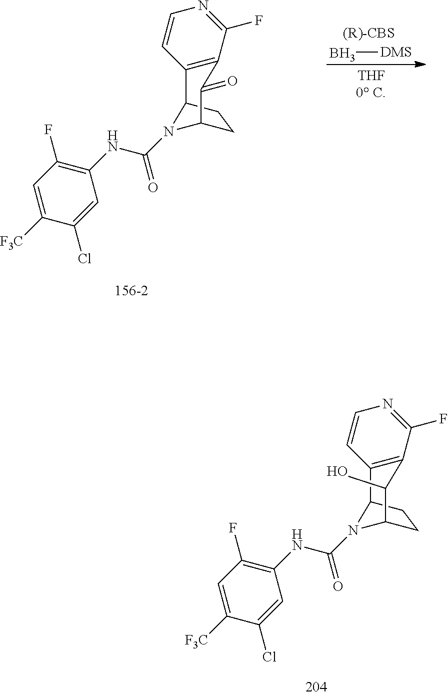

To a solution of (±)—N-(5-chloro-2-fluoro-4-(trifluoromethyl)phenyl)-1-fluoro-9-oxo-6,7,8,9-tetrahydro-5H-5,8-epiminocyclohepta[c]pyridine-10-carboxamide 156-2 (500 mg, 1.2 mmol) and (R)—CBS (133 mg, 0.48 mmol) in THF (20 ml) was added borane-dimethyl sulfide complex (0.3 ml, 10 M in dimethyl sulfide) at 0° C. under N₂. The reaction mixture was stirred at 0° C. for 1 h. The reaction mixture was diluted with water and extracted with EtOAc (3×30 ml). The combined organics were washed with brine, dried over Na₂SO₄ and concentrated in vacuo. The residue was purified by chromatography on silica gel (1% MeOH/DCM) and prep-TLC (3% MeOH/DCM) to give (±)—(R)—N-(5-chloro-2-fluoro-4-(trifluoromethyl)phenyl)-1-fluoro-9-hydroxy-6,7,8,9-tetrahydro-5H-5,8-epiminocyclohepta[c]pyridine-10-carboxamide 204 as a white solid. LCMS (Method 3) m/z 432 (M−H)⁻ (ES)⁻ at 3.15 min. ¹H NMR (400 MHz, DMSO-d6) δ 8.87 (s, 1H), 8.12-8.08 (m, 2H), 7.79 (d, J=11.2 Hz, 1H), 7.28 (d, J=6.8 Hz, 1H), 6.26 (s, 1H), 5.31 (d, J=6.0 Hz, 1H), 4.78 (d, J=8.4 Hz, 1H), 4.53 (s, 1H), 2.22-2.15 (m, 1H), 2.07-2.03 (m, 1H), 1.60-1.50 (m, 2H).

### Experimental Scheme 25

### Compound 225: (6S,9R)—N-(5-Chloro-2-fluoro-4-((6-fluoropyridin-3-yl)oxy)phenyl)-3-oxo-3,5,6,7,8,9-hexahydro-2H-6,9-epiminocyclohepta[c]pyridazine-10-carboxamide

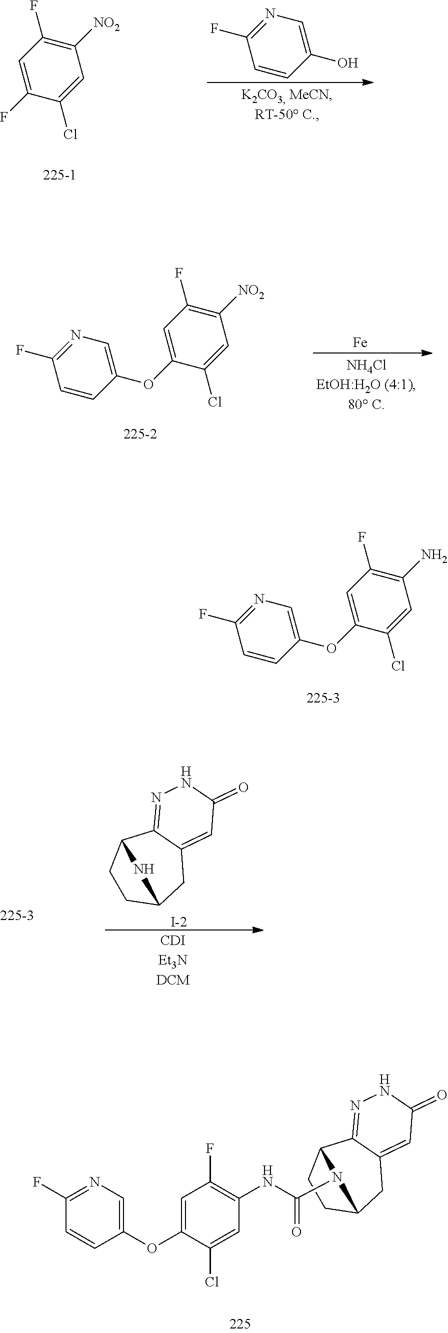

Step 1: To a solution of 1-chloro-2,4-difluoro-5-nitrobenzene 225-1 (500 mg, 2.59 mmol) in MeCN (10 ml) were added 6-fluoropyridin-3-ol (292.7 mg, 2.59 mmol) and K₂CO₃ (1.07 g, 7.77 mmol). After stirring for 2 h at RT, the mixture was heated at 50° C. for 1 h. After this time the mixture was concentrated in vacuo and the product was purified by chromatography on silica gel (2% EtOAc/petroleum ether) to give 5-(2-chloro-5-fluoro-4-nitrophenoxy)-2-fluoropyridine 225-2 as a colourless oil. LCMS (Method 5) m/z 287.1 (M+H)⁺ (ES)⁺ at 1.61 min, ¹H NMR (400 MHz, DMSO-d6) δ 8.47-8.44 (m, 1H), 8.22-8.21 (m, 1H), 7.95-7.90 (m, 1H), 7.32-7.29 (m, 2H).

Step 2: To a solution of 5-(2-chloro-5-fluoro-4-nitrophenoxy)-2-fluoropyridine 225-2 (547 mg, 1.9 mmol) in EtOH (6 ml) were added Fe (856.8 mg, 15.3 mmol), NH₄Cl (1.026 g, 19 mmol) and water (1.5 ml). The resultant mixture was stirred at 80° C. for 1 h. The mixture was concentrated in vacuo and the product was purified by prep-TLC (10% EtOAc/petroleum ether) to give 5-chloro-2-fluoro-4-((6-fluoropyridin-3-yl)oxy)aniline 225-3 as a yellow solid. LCMS (Method 5) m/z 257.0 (M+H)⁺ (ES)⁺ at 1.61 min. ¹H NMR (400 MHz, DMSO-d6) δ 7.87-7.86 (m, 1H), 7.51-7.46 (m, 1H), 7.15-7.14 (m, 1H), 7.13-7.12 (m, 1H), 6.94-6.92 (d, J=9.2 Hz, 1H), 5.43 (s, 2H).

Step 3: To a solution of 5-chloro-2-fluoro-4-((6-fluoropyridin-3-yl)oxy)aniline 225-3 (50 mg, 0.195 mmol) in DCM (1 ml) was added CDI (47.5 mg, 0.293 mmol). The resultant mixture was stirred at RT for 30 min. A solution of (6S,9R)-2,5,6,7,8,9-hexahydro-3H-6,9-epiminocyclohepta[c]pyridazin-3-one I-2 (69 mg, 0.293 mmol) and Et₃N (54.3 ml, 0.39 mmol) in DCM (1 ml) were added. The reaction was stirred at RT for 16 h. The mixture was concentrated in vacuo and the residue was purified by prep-TLC (DCM/MeOH=20/1, v/v) to give (6S,9R)—N-(5-chloro-2-fluoro-4-((6-fluoropyridin-3-yl)oxy)phenyl)-3-oxo-3,5,6,7,8,9-hexahydro-2H-6,9-epiminocyclohepta[c]pyridazine-10-carboxamide as a white solid. LCMS (Method 3) m/z 460.1 (M+H)⁺ (ES)⁺ at 2.51 min. ¹H NMR (400 MHz, DMSO-d6) δ 12.70 (s, 1H), 8.78 (s, 1H), 8.02-8.00 (m, 1H), 7.75 (d, J=8.0 Hz, 1H), 7.69-7.64 (m, 1H), 7.23-7.20 (m, 2H), 6.67 (s, 1H), 5.04 (d, J=5.6 Hz, 1H), 4.62-4.60 (m, 1H), 3.21-3.15 (m, 1H), 2.64 (d, J=18.4 Hz, 1H), 2.25-2.12 (m, 2H), 1.81-1.65 (m, 1H), 1.29-1.24 (m, 1H).

### Experimental Scheme 26

### Compound 283: (5R,8S)—N-(4,5-Dichloro-2-fluorophenyl)-1-fluoro-4-(2-methoxyethoxy)-6,7,8,9-tetrahydro-5H-5,8-epiminocyclohepta[c]pyridine-10-carboxamide

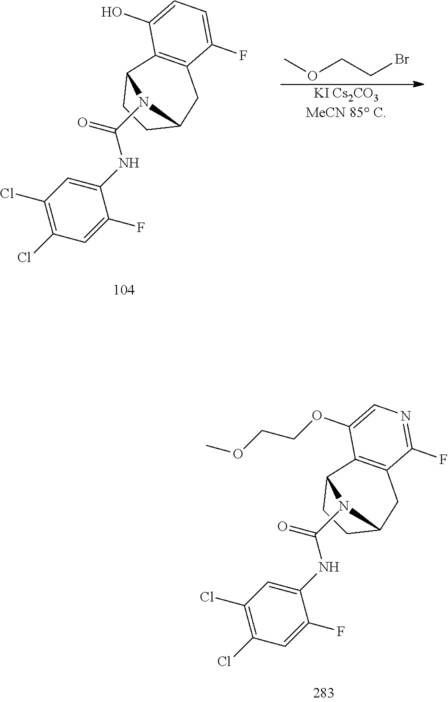

A mixture of (5R,8S)—N-(4,5-dichloro-2-fluorophenyl)-1-fluoro-4-hydroxy-6,7,8,9-tetrahydro-5H-5,8-epiminocyclohepta[c]pyridine-10-carboxamide 104 (55 mg, 0.14 mmol), 1-bromo-2-methoxyethane (58.4 mg, 0.42 mmol), KI (2.3 mg, 0.014 mmol) and Cs₂CO₃ (91.2 mg, 0.28 mmol) in MeCN (2.5 ml) was stirred at 80° C. for 16 h. The mixture was diluted with water (20 ml) and the product was extracted with EtOAc (3×20 ml). The combined organics were washed with brine (30 ml), dried over Na₂SO₄ and concentrated in vacuo. The product was purified by prep-TLC (50% EtOAc/petroleum ether) to afford (5R,8S)—N-(4,5-dichloro-2-fluorophenyl)-1-fluoro-4-(2-methoxyethoxy)-6,7,8,9-tetrahydro-5H-5,8-epiminocyclohepta[c]pyridine-10-carboxamide 283 as a white solid. LCMS (Method 3) m/z 458.1 (M+H)⁺ (ES)⁺ at 3.60 min, ¹H NMR (400 MHz, DMSO-d6) δ 8.85 (s, 1H), 7.85-7.82 (m, 1H), 7.74 (s, 1H), 7.67 (d, J=10.4 Hz, 1H), 5.37 (d, J=6.0 Hz, 1H), 4.75 (t, J=12.0 Hz, 1H), 4.27-4.23 (m, 2H), 3.72-3.69 (m, 2H), 3.32 (s, 3H), 3.17 (dd, J=4.8 Hz, 4.8 Hz, 1H), 2.55-2.51 (m, 1H), 2.32-2.11 (m, 2H), 1.81-1.70 (m, 2H).

### Experimental Scheme 27

### Compound 285: (5R,8S)—N-(5-cyclohexyl-1,3,4-thiadiazol-2-yl)-1-fluoro-6,7,8,9-tetrahydro-5H-5,8-epiminocyclohepta[c]pyridine-10-carboxamide

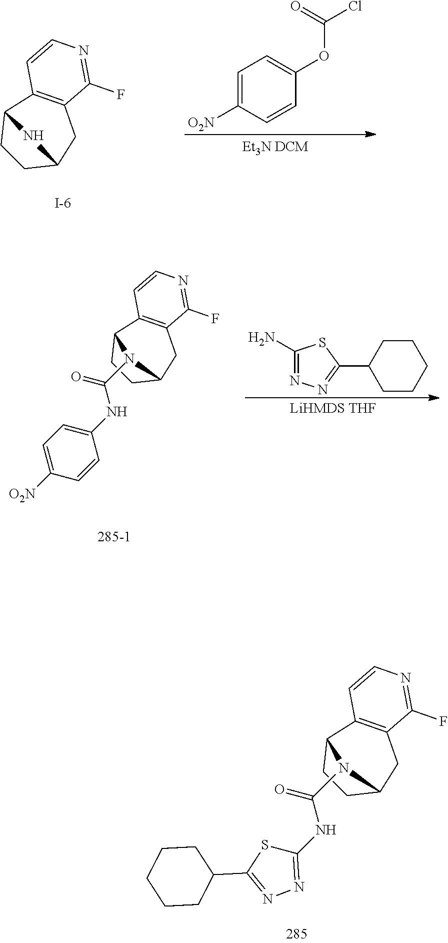

Step 1: To a solution of (5R,8S)-1-fluoro-6,7,8,9-tetrahydro-5H-5,8-epiminocyclohepta[c]pyridine I-6 (750 mg, 4.21 mmol) in DCM (10 ml) was added Et₃N (1.47 ml, 10.5 mmol). The resultant solution was cooled to 0° C. and 4-nitrophenyl carbonochloridate (1.27 g, 6.31 mmol) was added as a single portion. The mixture was allowed to warm to RT for 16 h. Water (20 ml) was added and the layers were separated. The aqueous was extracted with DCM (2×10 ml). The combined organics were washed with water (20 ml), dried over MgSO₄, filtered and concentrated in vacuo. The product was purified by chromatography on silica gel (0-80% EtOAc/isohexane) to afford 4-nitrophenyl (5R,8S)-1-fluoro-6,7,8,9-tetrahydro-5H-5,8-epiminocyclohepta[c]pyridine-10-carboxylate 265-1 as a brown powder. ¹H NMR (400 MHz, DMSO-d6) δ 8.25 (d, J=8.6 Hz, 2H), 8.05 (d, J=5.0 Hz, 1H), 7.42 (dd, J=44.3, 8.7 Hz, 2H), 7.30 (d, J=5.0 Hz, 1H), 5.49-5.05 (m, 1H), 4.75 (d, J=64.9 Hz, 1H), 3.31-3.08 (m, 1H), 2.70 (dd, J=17.6, 8.6 Hz, 1H), 2.41-2.21 (m, 2H), 1.97-1.70 (m, 2H).

Step 2: To a solution of 5-cyclohexyl-1,3,4-thiadiazol-2-amine (16 mg, 87 μmol) in THF (1.5 ml) was added a solution of LiHMDS in THF (0.17 ml, 1 M, 0.17 mmol) at RT and the reactions were stirred for 1 h. After this time a solution of 4-nitrophenyl (5R,8S)-1-fluoro-6,7,8,9-tetrahydro-5H-5,8-epiminocyclohepta[c]pyridine-10-carboxylate 265-1 (30 mg, 87 μmol) in THF (0.5 ml) was added and the mixtures were stirred at RT for 16 h. A saturated solution of NaHCO₃ (30 ml) was added and the product was extracted with 10% MeOH in ethyl acetate. The organics were then washed with brine, dried over MgSO₄ and dried in vacuo. The product was purified by chromatography on silica gel (0-50% (10% MeOH in ethyl acetate) in isohexane) to afford (5R,8S)—N-(5-cyclohexyl-1,3,4-thiadiazol-2-yl)-1-fluoro-6,7,8,9-tetrahydro-5H-5,8-epiminocyclohepta[c]pyridine-10-carboxamide 285 as a white solid.

### Experimental Scheme 28

### Compound 226: (±, 9-endo)-N-(5-chloro-2-fluoro-4-(trifluoromethyl)phenyl)-1,9-difluoro-6,7,8,9-tetrahydro-5H-5,8-epiminocyclohepta[c]pyridine-10-carboxamide

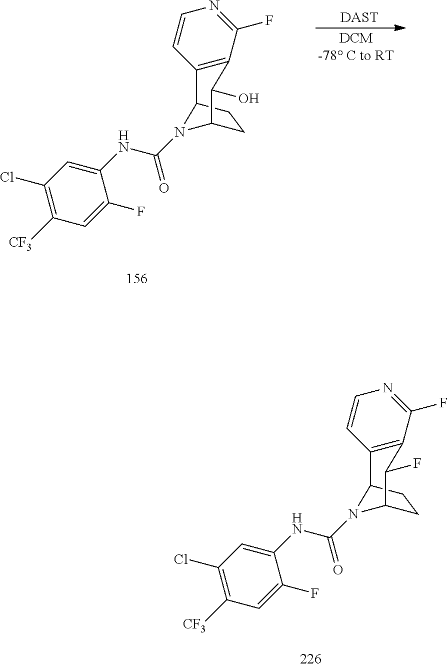

To a solution of (±)-(9-endo)-N-(5-chloro-2-fluoro-4-(trifluoromethyl)phenyl)-1-fluoro-9-hydroxy-6,7,8,9-tetrahydro-5H-5,8-epiminocyclohepta[c]pyridine-10-carboxamide (320 mg, 0.74 mmol) in DCM (5 ml) was added DAST (715.7 mg, 4.44 mmol) at −78° C. under N₂. The reaction mixture was allowed to warm to RT and stirred for 16 h. The mixture was basified with saturated NaHCO₃ aqueous solution and extracted with DCM (3×30 ml). The combined organics were dried over Na₂SO₄ and concentrated in vacuo. The product was purified by prep-TLC (33% EtOAc/petroleum ether) to (±, 9-endo)-N-(5-chloro-2-fluoro-4-(trifluoromethyl)phenyl)-1,9-difluoro-6,7,8,9-tetrahydro-5H-5,8-epiminocyclohepta[c]pyridine-10-carboxamide 226 as a white solid and (±)-(9-exo)-N-(5-Chloro-2-fluoro-4-(trifluoromethyl)phenyl)-1,9-difluoro-6,7,8,9-tetrahydro-5H-5,8-epiminocyclohepta[c]pyridine-10-carboxamide 157 as a white solid.

(±, 9-endo)-N-(5-chloro-2-fluoro-4-(trifluoromethyl)phenyl)-1,9-difluoro-6,7,8,9-tetrahydro-5H-5,8-epiminocyclohepta[c]pyridine-10-carboxamide 226. LCMS (Method 3) m/z 434.1 (M+H)⁺ (ES)⁺ at 3.43 min, ¹H NMR (400 MHz, DMSO-d6) δ 9.27 (s, 1H), 8.20 (d, J=5.2 Hz, 1H), 8.03 (d, J=6.8 Hz, 1H), 7.81 (d, J=10.8 Hz, 1H), 7.34 (d, J=5.2 Hz, 1H), 6.29 (dd, J=51.0, 5.8 Hz, 1H), 5.34 (d, J=6.0 Hz, 1H), 5.01 (m, 1H), 2.21-2.14 (m, 3H), 1.83-1.80 (m, 1H).

### Experimental Scheme 29

### Compound 231: (6S,9R)—N-(5-Chloro-2-fluoro-4-(phenylcarbamoyl)phenyl)-3-oxo-3,5,6,7,8,9-hexahydro-2H-6,9-epiminocyclohepta[c]pyridazine-10-carboxamide

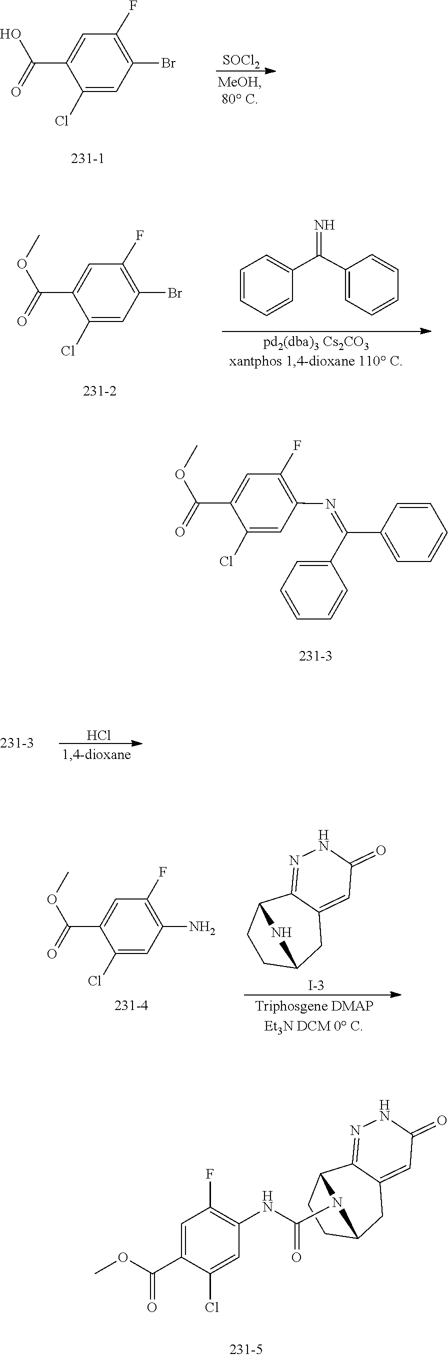

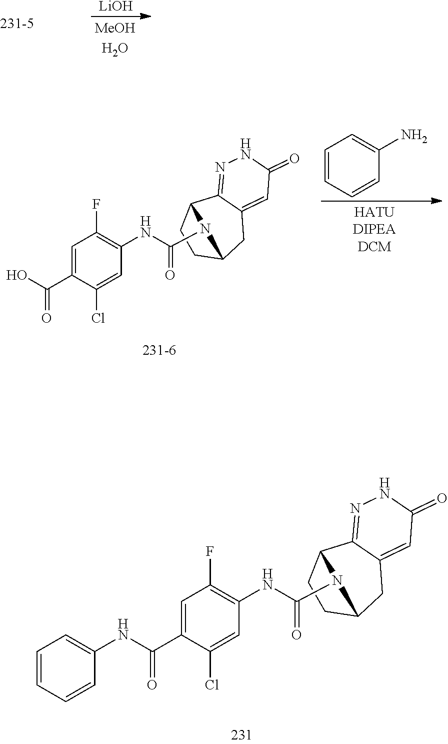

Step 1: To a solution of 4-bromo-2-chloro-5-fluorobenzoic acid 231-1 (500 mg, 1.97 mmol) in MeOH (5 ml) was added SOCl₂ (714 μl, 9.86 mmol) at RT. The reaction was refluxed for 16 h. After this time, water (10 ml) was added and the mixture was extracted with EtOAc (3×10 ml). The combined organics were washed with brine, dried over Na₂SO₄ and concentrated in vacuo. The product was purified by chromatography on silica gel (20% EtOAc/petroleum ether) to give methyl 4-bromo-2-chloro-5-fluorobenzoate 231-2 as a yellow oil. ¹H NMR (400 MHz, DMSO-d6) δ 8.06 (d, J=6 Hz, 1H), 7.83 (d, J=8.8 Hz, 1H), 3.86 (s, 3H).

Step 2: To a solution of methyl 4-bromo-2-chloro-5-fluorobenzoate 231-2 (294 mg, 1.09 mmol), diphenylmethanimine (199 mg, 1.09 mmol), xantphos (127 mg, 0.21 mmol) and Cs₂CO₃ (895 mg, 2.75 mmol) in 1,4-dioxane (5 ml) was added Pd₂(dba)₃ (113 mg, 0.10 mmol) under N₂. The reaction was stirred at 110° C. for 16 h. Water (10 ml) was added and the mixture was extracted with EtOAc (3×10 ml). The combined organics layers were washed with brine, dried with Na₂SO₄ and concentrated in vacuo. The product was purified by chromatography on silica gel (20% EtOAc/petroleum ether) to methyl 2-chloro-4-((diphenylmethylene)amino)-5-fluorobenzoate 231-3 as a yellow oil. ¹H NMR (400 MHz, DMSO-d6) δ 7.74-7.66 (m, 2H), 7.62-7.55 (m, 4H), 7.39 (s, 3H), 7.21 (s, 2H), 7.13 (d, J=7.2 Hz, 1H), 3.78 (s, 3H).

Step 3: To a solution of methyl 2-chloro-4-((diphenylmethylene)amino)-5-fluorobenzoate 231-3 (221 mg, 0.60 mmol) in 1,4-dioxane (2 ml) was added 2 M HCl (2 ml), the reaction was stirred at RT for 2 h. Water (10 ml) was added and the mixture was extracted with EtOAc (3×10 ml). The combined organics were washed with brine, dried with Na₂SO₄ and concentrated in vacuo. The product was purified by chromatography on silica gel (20% EtOAc/petroleum ether) to give methyl 4-amino-2-chloro-5-fluorobenzoate 231-4 as a yellow oil. ¹H NMR (400 MHz, DMSO-d6) δ 7.53 (d, J=12.4 Hz, 1H), 6.82 (d, J=8 Hz, 1H)), 6.28 (s, 2H), 3.75 (s, 3H),

Step 4: To a solution of methyl 4-amino-2-chloro-5-fluorobenzoate 231-4 (93 mg, 0.46 mmol) and triphosgene (68 mg, 0.23 mmol) in DCM (5 ml) was added a solution of DMAP (178 mg, 1.46 mmol) in DCM (1 ml) at 0° C. The mixture was stirred at 0° C. for 30 min. The solution was added to a solution of (6S,9R)-2,5,6,7,8,9-hexahydro-3H-6,9-epiminocyclohepta[c]pyridazin-3-one I-3 (266 mg, 0.91 mmol) and Et₃N (255 μl, 1.82 mmol) in DCM (1 ml). After stirring at RT for 16 h, the reaction was quenched with water (10 ml). The mixture was extracted with DCM (3×10 ml), the combined organics were dried over Na₂SO₄ and concentrated in vacuo. The product was purified by prep-TLC (10% EtOAc/petroleum ether) to give methyl 2-chloro-5-fluoro-4-((6S,9R)-3-oxo-3,5,6,7,8,9-hexahydro-2H-6,9-epiminocyclohepta[c]pyridazine-10-carboxamido)benzoate 231-5 as a white solid.

Step 5: To a solution of methyl 2-chloro-5-fluoro-4-((6S,9R)-3-oxo-3,5,6,7,8,9-hexahydro-2H-6,9-epiminocyclohepta[c]pyridazine-10-carboxamido)benzoate 231-5 (137 mg, 0.34 mmol) in MeOH (3 ml) and water (1 ml) was added LiOH (28 mg, 0.67 mmol). After stirring at RT for 16 h, the reaction mixture was acidified with 1N HCl solution and extracted with EtOAc (3×10 ml). The combined organics were washed with brine, dried with Na₂SO₄ and concentrated in vacuo. The product was purified by silica gel column (10% MeOH/DCM) to give 2-chloro-5-fluoro-4-((6S,9R)-3-oxo-3,5,6,7,8,9-hexahydro-2H-6,9-epiminocyclohepta[c]pyridazine-10-carboxamido)benzoic acid 231-6 as a yellow oil. LCMS (Method 3) m/z 392.9 (M+H)⁺ (ES)⁺ at 0.74 min

Step 6: To a solution of 2-chloro-5-fluoro-4-((6S,9R)-3-oxo-3,5,6,7,8,9-hexahydro-2H-6,9-epiminocyclohepta[c]pyridazine-10-carboxamido)benzoic acid 231-6 (132 mg, 0.33 mmol) in DCM (5 ml) were added aniline (31 mg, 0.33 mmol), HATU (153 mg, 0.40 mmol) and DIPEA (179 μl, 1.00 mmol). After stirring at RT for 16 h, water (10 ml) was added and the mixture was extracted with EtOAc (3×10 ml). The combined organics were washed with brine, dried with Na₂SO₄ and concentrated in vacuo. The product was purified by chromatography on silica gel (33% EtOAc/petroleum ether) to give (6S,9R)—N-(5-chloro-2-fluoro-4-(phenylcarbamoyl)phenyl)-3-oxo-3,5,6,7,8,9-hexahydro-2H-6,9-epiminocyclohepta[c]pyridazine-10-carboxamide 231 as a yellow solid. LCMS (Method 3) m/z 467.9 (M+H)⁺ (ES)⁺ at 2.47 min. ¹H NMR (400 MHz, DMSO-d6) δ 12.72 (s, 1H), 10.44 (s, 1H), 8.92 (s, 1H), 7.74 (d, J=6 Hz, 1H), 7.68 (d, J=8 Hz, 2H), 7.52 (d, J=10.4 Hz, 1H), 7.34 (t, J=7.6 Hz, 2H), 7.10 (t, J=7.2 Hz, 1H), 6.69 (s, 1H), 3.21-3.16 (m, 1H), 2.68-2.63 (m, 1H), 2.23-2.13 (m, 2H), 1.84-1.77 (m, 1H), 1.71-1.66 (m, 1H).

### Experimental Scheme 30

### Compound 236: (6S,9R)—N-(4-Benzamido-5-chloro-2-fluorophenyl)-3-oxo-3,5,6,7,8,9-hexahydro-2H-6,9-epiminocyclohepta[c]pyridazine-10-carboxamide

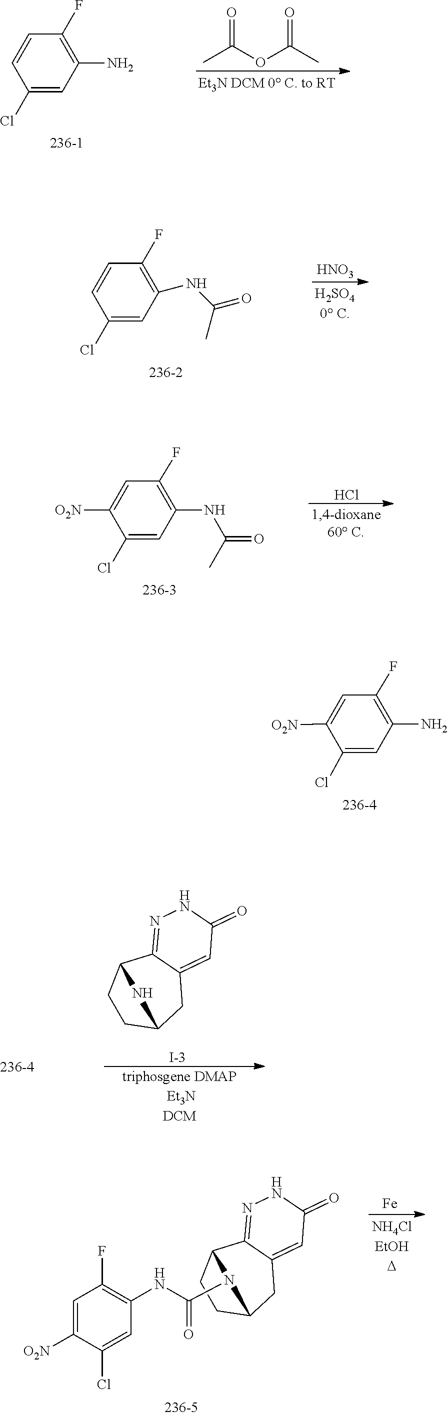

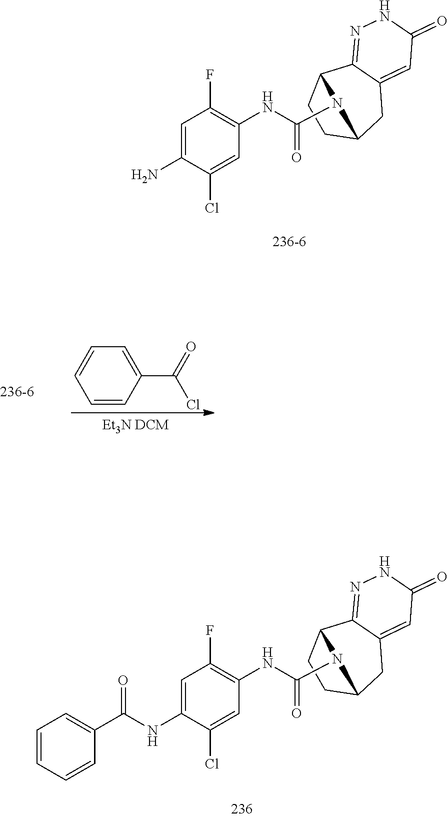 [SMILES: Nc1cc(F)c(NC(=O)N2[C@H]3CC[C@@H]2c2n[nH]c(=O)cc2C3)cc1Cl ; O=C(Cl)c1ccccc1 ; O=C(Nc1cc(F)c(NC(=O)N2[C@H]3CC[C@@H]2c2n[nH]c(=O)cc2C3)cc1Cl)c1ccccc1]

Step 1: To a solution of 5-chloro-2-fluoroaniline 236-1 (300 mg, 2.07 mmol) and Et₃N (0.7 ml, 5.17 mmol) in DCM (5 ml) was added acetic anhydride (0.23 ml, 2.48 mmol) at 0° C. The mixture was warmed to RT for 16 h. The reaction mixture was diluted with water (10 ml) and extracted with DCM (2×10 ml). The combined organics were dried over Na₂SO₄ and concentrated in vacuo. The product was purified by chromatography on silica gel (20% EtOAc/petroleum ether) to give N-(5-chloro-2-fluorophenyl)acetamide 236-2 as a white solid. ¹H NMR (400 MHz, DMSO-d6) δ 9.89 (s, 1H), 8.08-8.06 (m, 1H), 7.32-7.19 (m, 1H), 7.19-7.15 (m, 1H), 2.10 (s, 3H).

Step 2: To a solution of HNO₃/H₂SO₄ (0.1 ml/2 ml) was added N-(5-chloro-2-fluorophenyl) acetamide 236-2 (370 mg, 1.98 mmol) at 0° C. The reaction was stirred at 0° C. for 30 min. The reaction mixture was adjusted the pH to 7-8 by addition of saturated aqueous NaHCO₃ and the product was extracted with EtOAc (2×10 ml). The combined organic layers were washed with brine (10 ml), dried over Na₂SO₄ and concentrated in vacuo. The product was purified by chromatography on silica gel (20% EtOAc/petroleum ether) to give N-(5-chloro-2-fluoro-4-nitrophenyl)acetamide 236-3 as a yellow solid. ¹H NMR (400 MHz, DMSO-d6) δ 10.37 (s, 1H), 8.53 (d, J=7.2 Hz, 1H), 8.22 (d, J=10.8 Hz, 1H), 2.18 (s, 3H).

Step 3: To a solution of N-(5-chloro-2-fluoro-4-nitrophenyl)acetamide 236-3 (350 mg, 1.5 mmol) in 1,4-dioxane (5 ml) was added concentrated HCl (5 ml). The reaction was stirred at 60° C. for 1 h in a sealed tube. The reaction mixture pH was adjusted to pH 7-8 by addition of saturated aqueous NaHCO₃ and the aqueous layer was extracted with DCM (2×10 ml). The combined organics were dried over Na₂SO₄ and concentrated in vacuo to give 5-chloro-2-fluoro-4-nitroaniline 236-4 as a yellow solid. ¹H NMR (400 MHz, DMSO-d6) δ 7.98 (d, J=11.6 Hz, 1H), 6.90-6.87 (m, 3H).

Step 4: To a solution of 5-chloro-2-fluoro-4-nitroaniline 236-4 (290 mg, 1.53 mmol) and triphosgene (227 mg, 0.76 mmol) in DCM (5 ml) was added a solution of DMAP (596 mg, 4.88 mmol) in DCM (3 ml) at 0° C. The resulting mixture was stirred at 0° C. for 30 mins. The reaction mixture was added to a solution of (6S,9R)-2,5,6,7,8,9-hexahydro-3H-6,9-epiminocyclohepta[c]pyridazin-3-one I-3 (268 mg, 1.51 mmol) and Et₃N (842 μl, 6.06 mmol) in DCM (5 ml). The reaction mixture was stirred at RT for 16 h. The reaction mixture was diluted with water (10 ml) and the product was extracted with DCM (2×10 ml). The combined organics were dried over Na₂SO₄ and concentrated in vacuo. The product was purified by chromatography on silica gel (2% MeOH/DCM) to give (6S,9R)—N-(5-chloro-2-fluoro-4-nitrophenyl)-3-oxo-3,5,6,7,8,9-hexahydro-2H-6,9-epiminocyclohepta[c]pyridazine-10-carboxamide 236-5 as a yellow solid. ¹H NMR (400 MHz, DMSO-d6) δ 12.74 (s, 1H), 9.26 (s, 1H), 8.17-8.07 (m, 2H), 6.68 (s, 1H), 5.11-5.10 (m, 1H), 4.66 (t, J=5.6 Hz, 1H), 3.21-3.16 (m, 1H), 2.66 (d, J=18.4 Hz, 1H), 2.26-2.12 (m, 2H), 1.83-1.78 (m, 1H), 1.71-1.66 (m, 1H).

Step 5: To a solution of (6S,9R)—N-(5-chloro-2-fluoro-4-nitrophenyl)-3-oxo-3,5,6,7,8,9-hexahydro-2H-6,9-epiminocyclohepta[c]pyridazine-10-carboxamide 236-5 (240 mg 0.61 mmol) in EtOH (2 ml) was added saturated NH₄Cl (1 ml) and Fe (170 mg, 3.04 mmol). The reaction was stirred at 80° C. for 1 h. The reaction mixture was filtered and the filtrate was diluted with water (5 ml) before extraction with DCM (3×10 ml). The combined organics were dried over Na₂SO₄ and concentrated in vacuo to afford (6S,9R)—N-(4-amino-5-chloro-2-fluorophenyl)-3-oxo-3,5,6,7,8,9-hexahydro-2H-6,9-epiminocyclohepta[c]pyridazine-10-carboxamide 236-6 as a yellow solid. LCMS (Method 5) m/z 363.9 (M+H)⁺ (ES)⁺ at 0.60 min.

Step 6: To a solution of (6S,9R)—N-(4-amino-5-chloro-2-fluorophenyl)-3-oxo-3,5,6,7,8,9-hexahydro-2H-6,9-epiminocyclohepta[c]pyridazine-10-carboxamide 236-6 (70 mg, 0.19 mmol) and Et₃N (54 μl, 0.38 mmol) in DCM (5 ml) was added benzoyl chloride (22 μl, 0.19 mmol). The reaction was stirred at RT for 16 h. The reaction was diluted with water (5 ml) before the product was extracted with DCM (3×10 ml). The combined organics were dried over Na₂SO₄ and concentrated in vacuo. The product was purified by prep-TLC (10% MeOH/DCM) to give (6S,9R)—N-(4-benzamido-5-chloro-2-fluorophenyl)-3-oxo-3,5,6,7,8,9-hexahydro-2H-6,9-epiminocyclohepta[c]pyridazine-10-carboxamide 236 as a yellow solid. LCMS (Method 3) m/z 467.9 (M+H)⁺ (ES)⁺ at 2.40 min. ¹H NMR (400 MHz, DMSO-d6): 12.73 (s, 1H), 10.04 (s, 1H), 8.81 (s, 1H), 7.97 (d, J=7.6 Hz, 2H), 7.69-7.51 (m, 5H), 6.69 (s, 1H), 5.06 (d, J=4.8 Hz, 1H), 4.63 (t, J=5.6 Hz, 1H), 3.23-3.17 (m, 1H), 2.65 (d, J=18.4 Hz, 1H), 2.24-2.16 (m, 2H), 1.82-1.77 (m, 1H), 1.71-1.65 (m, 1H).

### Experimental Scheme 31

### Compound 287: (±)—N-(4,5-Dichloro-2-fluorophenyl)-N,1-dimethyl-2-oxo-2,5,6,7,8,9-hexahydro-1H-5,8-epiminocyclohepta[b]pyridine-10-carboxamide

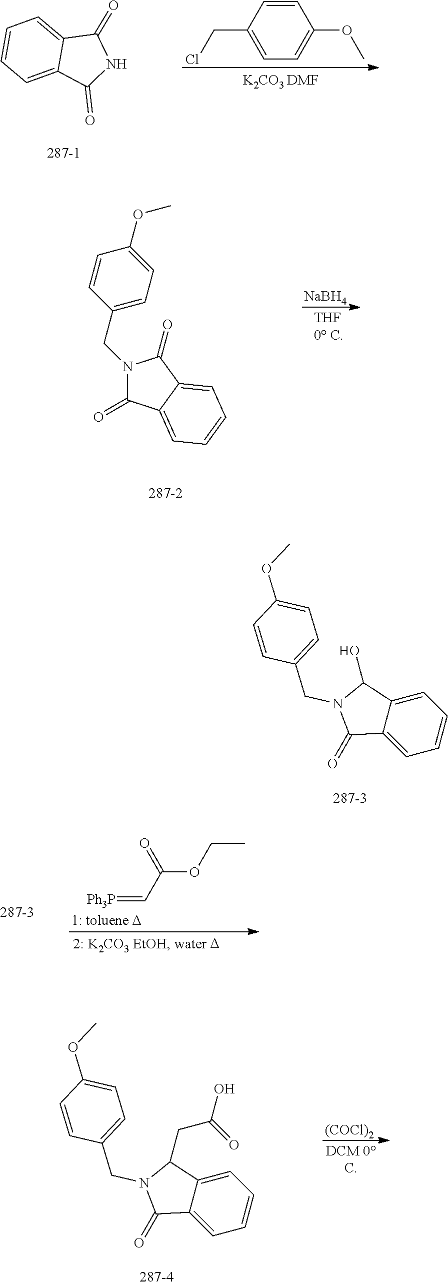

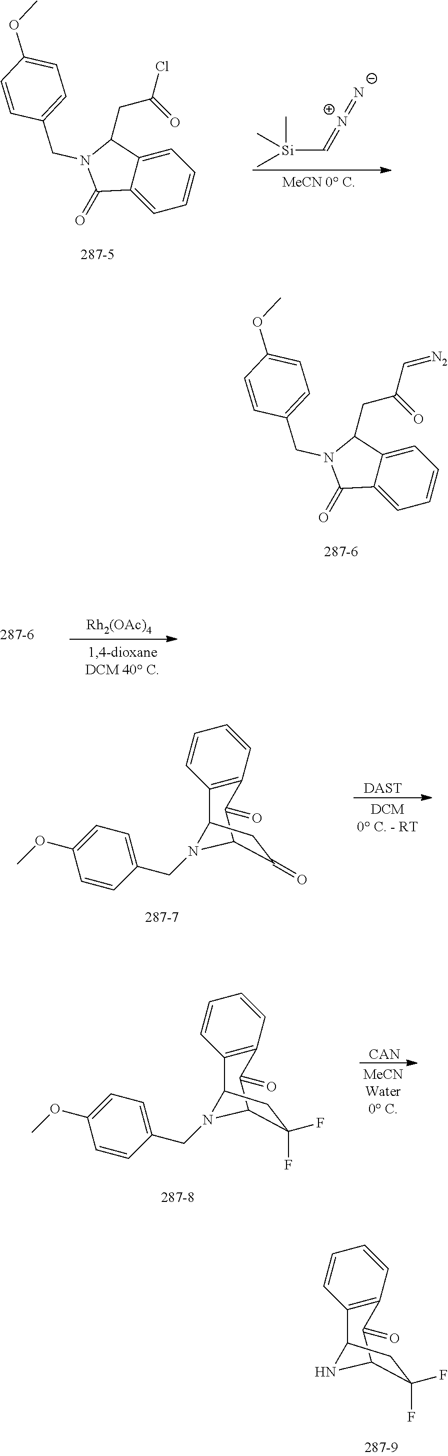

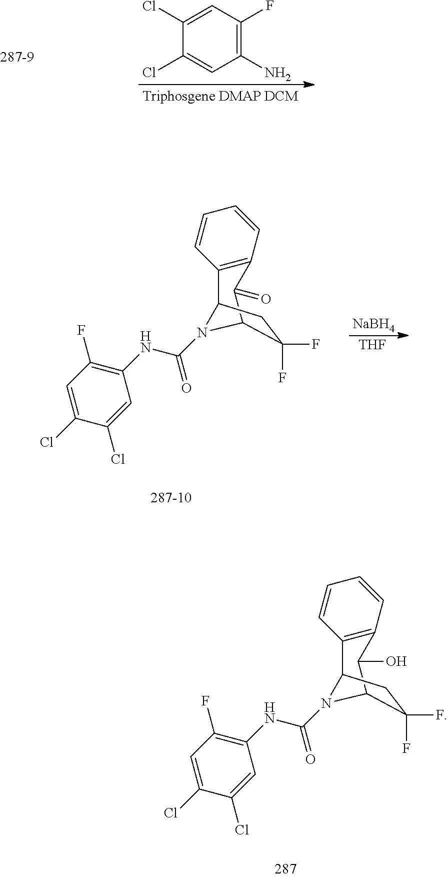

Step 1: To a solution of isoindoline-1,3-dione 287-1 (5 g, 34 mmol) and 1-(chloromethyl)-4-methoxybenzene (5.3 g, 34 mmol) in DMF (100 ml) at RT was added K₂CO₃ (14 g, 102 mmol). After stirred at RT for 16 h, the reaction mixture was concentrated in vacuo and the product was purified by chromatography on silica gel (50% EtOAc/petroleum ether) to give 2-(4-methoxybenzyl)isoindoline-1,3-dione 287-2 as a white solid. LCMS (Method 5) m/z 267.9 (M+H)⁺ (ES)⁺ at 1.36 min

Step 2: To a solution of 2-(4-methoxybenzyl)isoindoline-1,3-dione 287-2 (5 g, 18.7 mmol) in THF (50 ml) was added NaBH₄ (708 mg, 18.7 mmol) at 0° C. The reaction was warmed to RT and stirred for 16 h. MeOH was added to the reaction mixture and the mixture was concentrated in vacuo. The product was purified by chromatography on silica gel (50% EtOAc/petroleum ether) to give 3-hydroxy-2-(4-methoxybenzyl)isoindolin-1-one 287-3 as a white solid. LCMS (Method 5) m/z 270.0 (M+H)⁺ (ES)⁺ at 0.88 min

Step 3: To a solution of 3-hydroxy-2-(4-methoxybenzyl)isoindolin-1-one 287-3 (2 g, 7.4 mmol) in toluene (20 ml) was added ethyl 2-(triphenyl-I5-phosphanylidene)acetate (3.1 g, 8.9 mmol) at RT. The mixture was heated at 100° C. for 24 h. The reaction mixture was concentrated in vacuo and the residue was dissolved in water (10 ml) and EtOH (30 ml) and K₂CO₃ (1.3 g, 9.62 mmol) was added and the mixture was stirred at 100° C. for a further 16 h. The mixture was filtered and the filtrate was concentrated in vacuo. The product was purified by chromatography on silica gel (50% EtOAc/petroleum ether) to give 2-(2-(4-methoxybenzyl)-3-oxoisoindolin-1-yl)acetic acid 287-4 as a white solid. LCMS (Method 5) m/z 312.0 (M+H)⁺ (ES)⁺ at 0.90 min

Step 4: To a solution of 2-(2-(4-methoxybenzyl)-3-oxoisoindolin-1-yl)acetic acid 287-4 (700 mg, 2.25 mmol) in DCM (10 ml) was added oxalyl chloride (0.25 ml, 2.9 mmol) at 0° C. After stirring at RT for 3 h, the mixture was concentrated in vacuo to give 2-(2-(4-methoxybenzyl)-3-oxoisoindolin-1-yl)acetyl chloride as a yellow oil. LCMS (Method 5) m/z 326.1 (M+H)⁺ (ES)⁺ at 1.16 min

Step 5: To a solution of (diazomethyl)trimethylsilane (7.2 ml, 6 mmol) in MeCN (20 ml) was added a solution of methyl 2-(2-(4-methoxybenzyl)-3-oxoisoindolin-1-yl)acetate 287-4 (1.5 g, 4 mmol) in MeCN (10 ml) at 0° C. After stirring at RT for 2 h, the mixture was concentrated in vacuo. The product was purified by chromatography on silica gel (20% EtOAc/petroleum ether) to give 3-(3-diazo-2-oxopropyl)-2-(4-methoxybenzyl)isoindolin-1-one 287-5 as a yellow oil.

Step 6: To a solution of 3-(3-diazo-2-oxopropyl)-2-(4-methoxybenzyl)isoindolin-1-one 287-5 (900 mg, 2.68 mmol) in 1,4-dioxane (5 ml) was added a solution of [Rh(OAc)₂]₂ (118 mg, 0.268 mmol) in DCM (5 ml) at RT. After stirring at 40° C. for 2 h, the mixture was concentrated in vacuo. The product was purified by chromatography on silica gel (EtOAc/petroleum ether) to give 10-(4-methoxybenzyl)-5,6-dihydro-7H-5,8-epiminobenzo[7]annulene-7,9(8H)—dione 287-6 as a yellow oil. LCMS (Method 5) m/z 308.0 (M+H)⁺ (ES)⁺ at 1.39 min

Step 7: To a solution of 10-(4-methoxybenzyl)-5,6-dihydro-7H-5,8-epiminobenzo[7]annulene-7,9(8H)-dione 287-6 (270 mg, 0.88 mmol) in 1,4-dioxane (5 ml) was added DAST (709 mg, 4.39 mmol) at RT. The mixture was stirred at RT for 16 h. The reaction mixture was concentrated in vacuo and the product was purified by chromatography on silica gel (20% EtOAc/petroleum ether) to give 7,7-difluoro-10-(4-methoxybenzyl)-5,6,7,8-tetrahydro-9H-5,8-epiminobenzo[7]annulen-9-one 287-7 as a yellow oil. LCMS (Method 5) m/z 330.1 (M+H)⁺ (ES)⁺ at 1.95 min

Step 8: To a solution of 7,7-difluoro-10-(4-methoxybenzyl)-5,6,7,8-tetrahydro-9H-5,8-epiminobenzo[7]annulen-9-one 287-7 (130 mg, 0.39 mmol) in MeCN (8 ml) was added a solution of CAN (647 mg, 1.18 mmol) in water (5 ml) at 0° C. The resultant mixture was stirred at RT for 1 h. The mixture was diluted with saturated aqueous NaHCO₃ solution and extracted with EtOAc (3×5 ml). The combined organics were concentrated in vacuo. The product was purified by chromatography on silica gel (20% EtOAc/petroleum ether) to give 7,7-difluoro-5,6,7,8-tetrahydro-9H-5,8-epiminobenzo[7]annulen-9-one 287-8 as a yellow oil. LCMS (Method 5) m/z 210.1 (M+H)⁺ (ES)⁺ at 0.76 min

Step 9: To a solution of 4,5-dichloro-2-fluoroaniline (100 mg, 0.49 mmol) and triphosgene (66 mg, 0.22 mmol) in DCM (2 m) at 0° C. was added a solution of DMAP (185 mg, 1.76 mol) in DCM (1 ml). The mixture was stirred at 0° C. for 30 min before a solution of 7,7-difluoro-5,6,7,8-tetrahydro-9H-5,8-epiminobenzo[7]annulen-9-one 287-8 (100 mg, 0.47 mol) in DCM (2 ml) was added dropwise. The reaction mixture was stirred at RT for 16 h. The reaction mixture was poured into water and extracted with DCM (2×20 ml), the combined organics were washed with brine, dried over Na₂SO₄ and concentrated in vacuo. The product was purified by prep-TLC (20% EtOAc/petroleum ether) to give N-(4,5-dichloro-2-fluorophenyl)-7,7-difluoro-9-oxo-6,7,8,9-tetrahydro-5H-5,8-epiminobenzo[7]annulene-10-carboxamide 287-9 as a yellow solid. LCMS (Method 4) m/z 415.1, 417.1 (M+H)⁺ (ES)⁺ at 4.34 min. ¹H NMR (400 MHz, DMSO-d6) δ 9.42 (s, 1H), 7.91 (d, J=7.6 Hz, 1H), 7.73-7.70 (m, 1H), 7.67-7.63 (m, 2H), 7.54-7.50 (m, 2H), 5.75-5.69 (m, 1H), 5.11 (d, J=13.6 Hz, 1H), 3.15-3.01 (m, 1H), 2.55-2.46 (m, 1H).

Step 10: To a solution of N-(4,5-dichloro-2-fluorophenyl)-7,7-difluoro-9-oxo-6,7,8,9-tetrahydro-5H-5,8-epiminobenzo[7]annulene-10-carboxamide 287 (20 mg, 0.04 mol) in THF (3 ml) was added NaBH₄ (4.56 mg, 0.13 mmol). The reaction mixture was stirred at RT for 16 h. The reaction mixture was concentrated in vacuo and the product was purified by prep-TLC (20% EtOAc/petroleum ether) to give (±)—N-(4,5-dichloro-2-fluorophenyl)-N,1-dimethyl-2-oxo-2,5,6,7,8,9-hexahydro-1H-5,8-epiminocyclohepta[b]pyridine-10-carboxamide 287 as a white solid. LCMS (Method 4) m/z 417.1, 419.1 (M+H)⁺ (ES)⁺ at 4.10 min. ¹H NMR (400 MHz, DMSO-d6) δ 9.09 (s, 1H), 7.79 (d, J=7.6 Hz, 1H), 7.68 (d, J=10.0 Hz, 1H), 7.49-7.47 (m, 1H), 7.32-7.28 (m, 1H), 7.24-7.14 (m, 2H), 5.99 (d, J=6.8 Hz, 1H), 5.38-5.37 (m, 1H), 5.17 (br. s, 1H), 4.79-4.75 (m, 1H), 2.88-2.74 (m, 1H), 2.28-2.19 (m, 1H),

### Experimental Scheme 32

### Compound 288: (±)—N-(4,5-Dichloro-2-fluorophenyl)-N,1-dimethyl-2-oxo-2,5,6,7,8,9-hexahydro-1H-5,8-epiminocyclohepta[b]pyridine-10-carboxamide

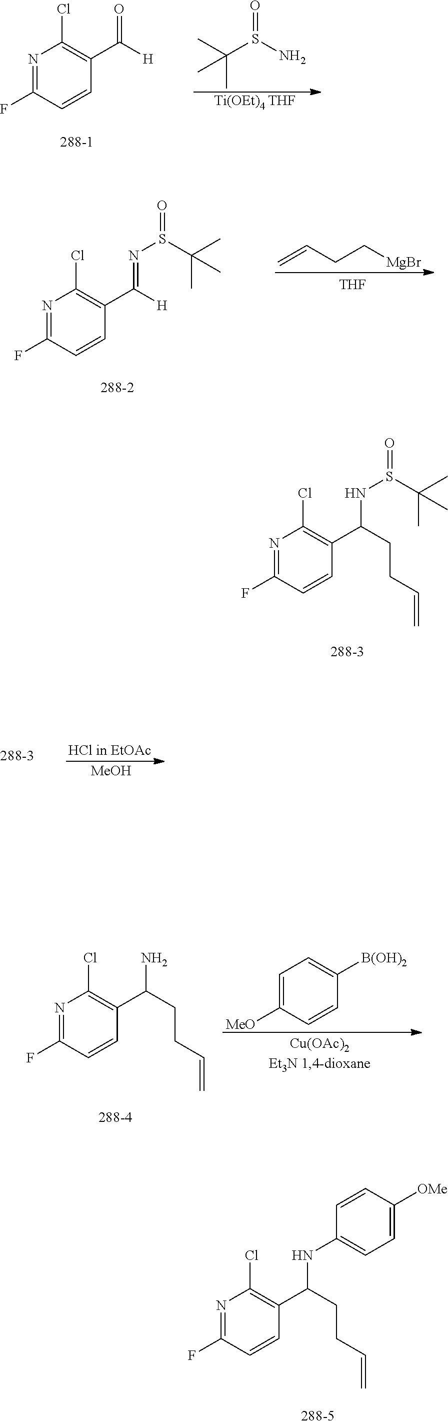

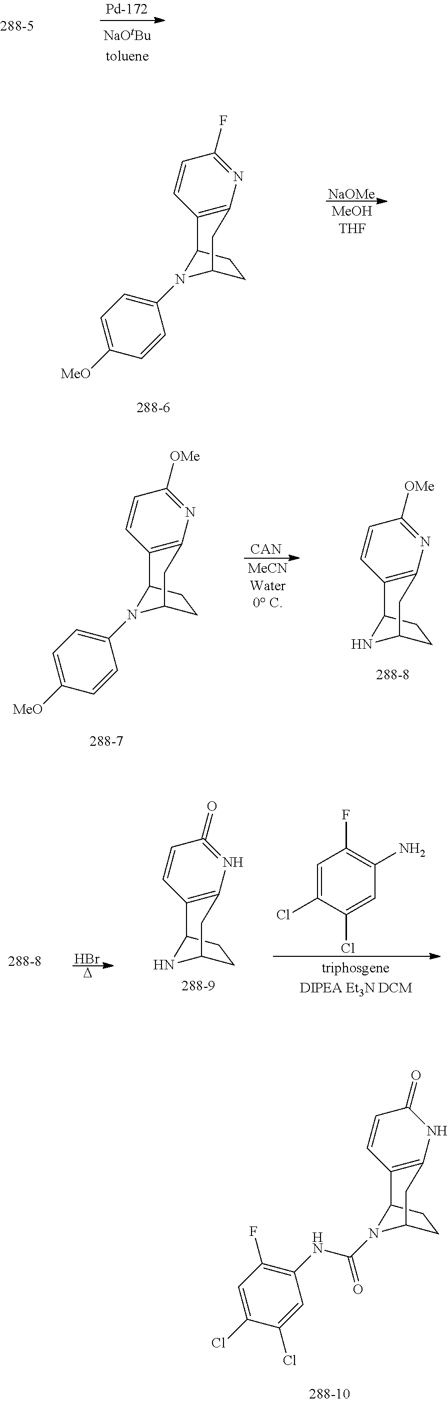

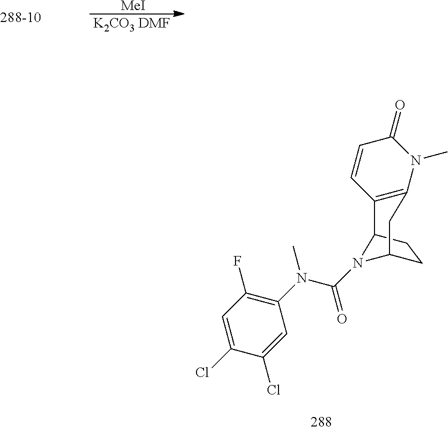

Step 1: (E)-N-((2-Chloro-6-fluoropyridin-3-yl)methylene)-2-methylpropane-2-sulfinamide (288-2) was synthesised from 2-chloro-6-fluoronicotinaldehyde (288-1) using a procedure essentially the same as for I-6b. ¹H NMR (400 MHz, CDCl₃) δ 8.93 (s, 1H), 8.48 (t, J=8.4 Hz, 1H), 7.02-6.99 (m, 1H), 1.28 (m, 9H).

Step 2: N-(1-(2-Chloro-6-fluoropyridin-3-yl)pent-4-en-1-yl)-2-methylpropane-2-sulfinamide (288-3) was synthesised from (E)-N-((2-chloro-6-fluoropyridin-3-yl)methylene)-2-methylpropane-2-sulfinamide (288-2) using a procedure essentially the same as for I-7c. ¹H NMR (400 MHz, CDCl₃) δ 7.82-7.87 (m, 1H), 6.87-6.90 (m, 1H), 5.76 (t, J=10.4 Hz, 1H), 5.01-5.05 (m, 2H), 4.98-4.84 (m, 1H), 5.82-3.79 (m, 1H), 2.20-2.05 (m, 2H), 1.94-1.88 (m, 2H) 1.16-1.22 (m, 9H)

Step 3: 1-(2-Chloro-6-fluoropyridin-3-yl)pent-4-en-1-amine (288-4) was synthesised from N-(1-(2-Chloro-6-fluoropyridin-3-yl)pent-4-en-1-yl)-2-methylpropane-2-sulfinamide (288-3) using a procedure essentially the same as for I-6d. ¹H NMR (400 MHz, CDCl₃) δ 8.00 (t, J=8.4 Hz, 1H), 6.87-6.90 (m, 1H), 5.72-5.83 (m 1H), 5.96-5.05 (m, 2H), 4.36 (t, J=5.6 Hz, 1H) 2.09-2.15 (m, 2H), 1.65-1.69 (m, 2H), 1.48 (s, 2H).

Step 4: N-(1-(2-Chloro-6-fluoropyridin-3-yl)pent-4-en-1-yl)-4-methoxyaniline (288-5) was synthesised from 1-(2-chloro-6-fluoropyridin-3-yl)pent-4-en-1-amine (288-4) using a procedure essentially the same as for I-5e. ¹H NMR (400 MHz, CDCl₃) δ 7.88 (t, J=8.0 Hz, 1H), 6.78-6.83 (m, 1H), 6.70 (t, J=4.4 Hz, 2H), 6.40 (d, J=4.4 Hz, 2H), 5.80-5.87 (m, 1H), 5.01-5.05 (m, 2H), 3.70 (s, 3H), 2.19-2.31 (m, 2H), 1.77-1.92 (m, 2H), 1.43 (s, 1H) (1 exchangeable NH not observed)

Step 5: To a solution of N-(1-(2-chloro-6-fluoropyridin-3-yl)pent-4-en-1-yl)-4-methoxyaniline (288-5) (3.00 g, 9.35 mmol) in toluene (60 ml) was added NaO^{t}Bu (1.35 g, 14.0 mmol) and Pd-172 (567 mg, 935 umol) at 20° C. The mixture was stirred at 110° C. for 16 h. The reaction mixture was concentrated in vacuo. The product was purified by silica gel chromatography (EtOAc/petroleum ether 1%-100%) to give (±)-2-fluoro-10-(4-methoxyphenyl)-6,7,8,9-tetrahydro-5H-5,8-epiminocyclohepta[b]pyridine 288-6 as a yellow solid. ¹H NMR (400 MHz, CDCl₃) δ 7.55 (t, J=8.0 Hz, 1H), 6.68-6.77 (m, 5H), 4.72 (d, J=2.4 Hz, 1H), 4.52 (d, J=5.6 Hz, 1H), 3.71 (s, 3H), 3.28 (dd, J=8.8, 4.4 Hz, 1H), 2.53 (d, J=8.8 Hz, 1H), 2.42-2.56 (m, 3H), 1.82-1.96 (m, 2H).

Step 6: To a solution of (±)-2-fluoro-10-(4-methoxyphenyl)-6,7,8,9-tetrahydro-5H-5,8-epiminocyclohepta[b]pyridine 288-6 (413 mg, 1.45 mmol) in THF (2.5 ml) and MeOH (2.5 ml) was added sodium methoxide (235 mg, 4.36 mmol). The resultant mixture was stirred at 65° C. for 16 h. The reaction was cooled to RT and EtOAc (15 ml) was added followed by water (10 ml). The layers were separated and the aqueous was extracted with DCM (2×25 ml). The combined organics were washed with brine (15 ml) and dried with Na₂SO₄. The solvent was removed under reduced pressure to give (±)-2-methoxy-10-(4-methoxyphenyl)-6,7,8,9-tetrahydro-5H-5,8-epiminocyclohepta[b]pyridine 288-7 as a colourless solid. ¹H NMR (400 MHz, DMSO-d6) δ 7.56 (d, J=8.3 Hz, 1H), 6.84-6.74 (m, 2H), 6.74-6.64 (m, 2H), 6.53 (d, J=8.2 Hz, 1H), 4.78 (d, J=5.6 Hz, 1H), 4.49 (dd, J=7.0, 5.0 Hz, 1H), 3.70 (s, 3H), 3.61 (s, 3H), 3.07 (dd, J=17.8, 4.8 Hz, 1H), 2.40 (d, J=17.8 Hz, 1H), 2.34-2.15 (m, 2H), 1.84-1.69 (m, 2H).

Step 7: To a solution of (±)-2-methoxy-10-(4-methoxyphenyl)-6,7,8,9-tetrahydro-5H-5,8-epiminocyclohepta[b]pyridine 288-7 (424 mg, 1.43 mmol) in MeCN (21 ml) at 0° C. was added a solution of CAN (2.51 g, 4.58 mmol) in H₂O (21 ml) dropwise. After the addition the reaction was stirred at 0° C. for 1 h. 2 M NaOH (25 ml) was added and the mixture was filtered. DCM was added and the precipitate was washed with DCM and water. The layers were separated and the aqueous was extracted with DCM (3×30 ml). The combined organics were dried with MgSO₄ and concentrated in vacuo to give (±)-2-methoxy-6,7,8,9-tetrahydro-5H-5,8-epiminocyclohepta[b]pyridine 288-8 as an off white solid. ¹H NMR (400 MHz, DMSO-d6) δ 7.32 (d, J=8.2 Hz, 1H), 6.52-6.46 (m, 1H), 4.08 (d, J=5.3 Hz, 1H), 3.76 (s, 3H), 3.74 (d, J=6.3 Hz, 1H), 3.01 (dd, J=17.4, 5.1 Hz, 1H), 2.44 (d, J=17.3 Hz, 1H), 1.98-1.82 (m, 2H), 1.74-1.65 (m, 1H), 1.54-1.43 (m, 1H).

Step 8: A solution of (±)-2-methoxy-6,7,8,9-tetrahydro-5H-5,8-epiminocyclohepta[b]pyridine 288-8 (199 mg, 1.05 mmol) diluted in HBr (1.77 ml, 48% w/w in H₂O, 15.7 mmol) was heated at reflux for 16 h. The reaction was cooled to RT and diluted with MeOH (4 ml). SCX (4 g, 3.15 mmol) was added and the mixture was stirred at RT for 2 h. The suspension was filtered, washed with MeOH (20 ml), and the resin was eluted with 0.7 M ammonia in MeOH (50 ml). The eluted brown solution was concentrated in vacuo to obtain (±)-6,7,8,9-tetrahydro-5H-5,8-epiminocyclohepta[b]pyridin-2-ol 288-9 as a dark brown solid. ¹H NMR (400 MHz, DMSO-d6) δ 7.19 (d, J=9.1 Hz, 1H), 6.08 (d, J=9.2 Hz, 1H), 4.07 (d, J=5.3 Hz, 1H), 3.88-3.69 (m, 1H), 2.86 (dd, J=17.7, 4.9 Hz, 1H), 2.28 (d, J=17.7 Hz, 1H), 2.07-1.81 (m, 2H), 1.83-1.68 (m, 1H), 1.62-1.43 (m, 1H). 2 exchangeable protons not observed.

Step 9: To a solution of 4,5-dichloro-2-fluoroaniline (189 mg, 1.05 mmol) in DCM (1 ml) was added Et₃N (400 μl, 2.86 mmol) followed by dropwise addition of a solution of triphosgene (127 mg, 429 μmol) in DCM (0.5 ml). The mixture was stirred at RT for five min before it was added to a solution of (±)-6,7,8,9-tetrahydro-5H-5,8-epiminocyclohepta[b]pyridin-2-ol 288-9 (168 mg, 953 μmol) and DIPEA (490 μl, 2.86 mmol) in DCM (1 ml). The reaction mixture was stirred at RT for 2 h. The reaction mixture was concentrated in vacuo. The product was purified by chromatography on silica gel (0-60% (0.7 M Ammonia/MeOH)/DCM). The product fractions were concentrated in vacuo. The residue was dissolved in DCM (10 ml) and washed with water (3 ml) and brine (3 ml). The organics were dried with MgSO₄ and concentrated in vacuo to give (±)—N-(4,5-dichloro-2-fluorophenyl)-2-oxo-2,5,6,7,8,9-hexahydro-1H-5,8-epiminocyclohepta[b]pyridine-10-carboxamide 288-10 as an off-white powder. ¹H NMR (400 MHz, DMSO-d6) δ 8.69 (s, 1H), 7.84 (d, J=7.6 Hz, 1H), 7.67 (dd, J=10.3, 4.0 Hz, 1H), 7.26 (d, J=9.2 Hz, 1H), 6.11 (d, J=9.2 Hz, 1H), 4.93 (d, J=5.6 Hz, 1H), 4.61 (t, J=6.3 Hz, 1H), 3.17 (d, J=5.2 Hz, 1H), 2.38 (s, 1H), 2.22-2.13 (m, 1H), 2.04 (dt, J=11.3, 5.4 Hz, 1H), 1.81 (t, J=9.8 Hz, 1H), 1.69 (t, J=10.2 Hz, 1H). 1 exchangeable proton not observed

Step 10: To a solution of (±)—N-(4,5-dichloro-2-fluorophenyl)-2-oxo-2,5,6,7,8,9-hexahydro-1H-5,8-epiminocyclohepta[b]pyridine-10-carboxamide 288-10 (115 mg, 301 μmol) in DMF (4 ml) was added iodomethane (20 μl, 301 μmol) and K₂CO₃ (125 mg, 903 μmol). The reaction mixture was stirred at RT for 16 h. A further portion of iodomethane (20 μl, 301 μmol) was added and stirring was continued at RT for 16 h. After this time saturated NaHCO₃ solution (10 ml) was added and the product was extracted with 10% MeOH/DCM (3×20 ml). The combined organic layers were dried over MgSO₄ and concentrated in vacuo. The product was purified by chromatography on RP Flash C₁₈ (5-50% MeCN/10 mM Ammonium Bicarbonate) The product was further purified by chromatography on silica gel (0-10% (0.7 M Ammonia/MeOH)/DCM) to afford (±)—N-(4,5-dichloro-2-fluorophenyl)-N,1-dimethyl-2-oxo-2,5,6,7,8,9-hexahydro-1H-5,8-epiminocyclohepta[b]pyridine-10-carboxamide 288 as a light white powder. LCMS (Method 1) m/z 410.0, 411.9 (M+H)⁺ (ES)⁺ at 1.20 min. ¹H NMR (400 MHz, DMSO-d6) δ 7.76-7.67 (m, 2H), 7.02 (d, J=9.2 Hz, 1H), 6.14 (d, J=9.1 Hz, 1H), 4.42-4.30 (m, 2H), 3.29 (s, 3H), 3.09 (s, 3H), 2.99 (dd, J=17.4, 5.0 Hz, 1H), 2.59 (s, 1H), 2.01 (q, J=10.8 Hz, 1H), 1.92-1.82 (m, 1H), 1.71 (t, J=10.1 Hz, 1H), 1.64-1.56 (m, 1H).

### Experimental Scheme 33

### Compound 300: (5R,8S)—N-(5-Chloro-2-fluoro-4-(6-(2-(methylamino)ethoxy)pyridin-3-yl)phenyl)-1-fluoro-6,7,8,9-tetrahydro-5H-5,8-epiminocyclohepta[c]pyridine-10-carboxamide

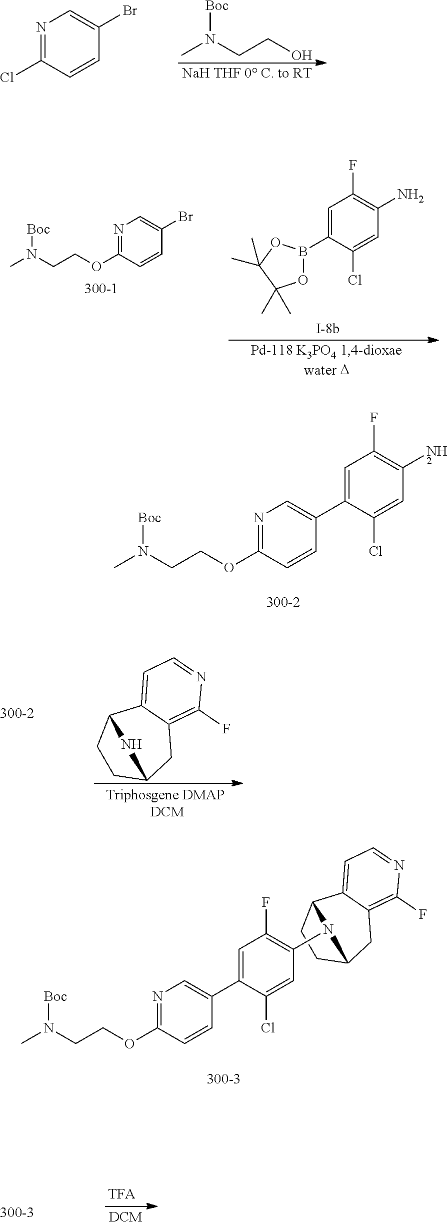

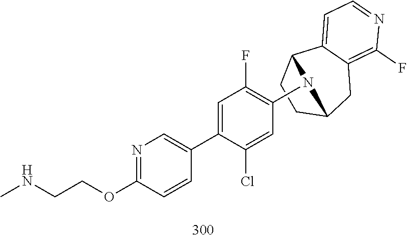

Step 1: To a solution of sodium hydride (55 mg, 60% w/w, 1.36 mmol) in THF (5 ml) at 0° C. was added tert-butyl (2-hydroxyethyl)(methyl)carbamate (201 μl, 1.19 mmol) and the resulting suspension stirred at 0° C. for 30 min before 5-bromo-2-fluoropyridine (117 μl, 1.14 mmol) was added. The reaction mixture was allowed to warm to RT and stirred at RT for 6 h before being quenched via addition of water. DCM (25 ml) and a 1:1 mixture of aqueous NaCl:NaHCO₃ (25 ml) was added and the layers were separated. The aqueous phase extracted with additional DCM (25 ml). The combined organics were concentrated in vacuo. The product was purified by chromatography on silica gel (0-50%, EtOAc/isohexane) to afford tert-butyl (2-((5-bromopyridin-2-yl)oxy)ethyl)(methyl)carbamate 300-1 as a colourless oil. ¹H NMR (400 MHz, DMSO-d6) δ 8.26 (d, J=2.6 Hz, 1H), 7.90 (d, J=10.3 Hz, 1H), 6.82 (d, J=8.4 Hz, 1H), 4.36 (t, J=5.4 Hz, 2H), 3.64-3.44 (m, 2H), 2.87-2.77 (m, 3H), 1.47-1.19 (m, 9H)

Step 2: tert-Butyl (2-((5-(4-amino-2-chloro-5-fluorophenyl)pyridin-2-yl)oxy)ethyl)(methyl)carbamate 300-2 was synthesised from 5-chloro-2-fluoro-4-(4,4,5,5-tetramethyl-1,3,2-dioxaborolan-2-yl)aniline I-8b and tert-butyl (2-((5-bromopyridin-2-yl)oxy)ethyl)(methyl)carbamate 300-1 using a procedure essentially the same as for I-47. ¹H NMR (400 MHz, DMSO-d6) δ 8.13 (dd, J=2.5, 0.7 Hz, 1H), 7.74 (dd, J=8.5, 2.5 Hz, 1H), 7.15-7.04 (m, 1H), 6.90 (d, J=8.3 Hz, 1H), 6.83 (d, J=8.6 Hz, 1H), 5.60 (s, 2H), 4.46-4.34 (m, 2H), 3.65-3.47 (m, 2H), 2.90-2.76 (m, 3H), 1.49-1.24 (m, 9H).

Step 3: To a solution of tert-butyl (2-((5-(4-amino-2-chloro-5-fluorophenyl)pyridin-2-yl)oxy)ethyl)(methyl)carbamate 300-2 (59.0 mg, 149 μmol) and DMAP (55 mg, 447 μmol) in DCM (4 ml) was added a solution of triphosgene (18 mg, 59.6 μmol) in DCM (2 ml). The resulting mixture was stirred at RT for 5 min. A solution of (5R,8S)-1-fluoro-6,7,8,9-tetrahydro-5H-5,8-epiminocyclohepta[c]pyridine I-6 (27 mg, 149 μmol) in DCM (2 ml) was added and the resultant mixture was stirred at RT for 18 h. The reaction mixture was concentrated in vacuo. The product was purified by chromatography on silica gel (0-50% EtOAc/isohexane) to afford tert-butyl (2-((5-(2-chloro-5-fluoro-4-((5R,8S)-1-fluoro-6,7,8,9-tetrahydro-5H-5,8-epiminocyclohepta[c]pyridine-10-carboxamido)phenyl)pyridin-2-yl)oxy)ethyl)(methyl)carbamate 300-3 as an off-white solid. ¹H NMR (400 MHz, DMSO-d6) δ 8.84 (s, 1H), 8.20 (d, J=2.8 Hz, 1H), 8.01 (d, J=5.0 Hz, 1H), 7.81 (d, J=8.1 Hz, 1H), 7.74 (d, J=7.4 Hz, 1H), 7.41-7.29 (m, 1H), 7.21 (d, J=4.3 Hz, 1H), 6.88 (d, J=8.3 Hz, 1H), 5.22 (d, J=5.7 Hz, 1H), 4.84-4.76 (m, 1H), 4.46-4.37 (m, 2H), 3.62-3.49 (m, 2H), 3.24-3.15 (m, 1H), 2.91-2.81 (m, 3H), 2.59 (d, J=17.3 Hz, 1H), 2.28-2.14 (m, 2H), 1.89-1.71 (m, 2H), 1.45-1.20 (m, 9H).

Step 4: To a solution of tert-butyl (2-((5-(2-chloro-5-fluoro-4-((5R,8S)-1-fluoro-6,7,8,9-tetrahydro-5H-5,8-epiminocyclohepta[c]pyridine-10-carboxamido)phenyl)pyridin-2-yl)oxy)ethyl)(methyl)carbamate 300-3 (37.0 mg, 61.7 μmol) in DCM (2 ml) was added TFA (50 μl, 0.6 mmol) slowly. The reaction mixture was stirred at RT for 1 h. The reaction mixture was added to SCX. The SCX washed with DCM (12 ml) and the product was eluted with 0.7 M NH₃ in MeOH (20 ml). The filtrate was concentrated in vacuo. The resultant solid was dissolved in EtOAc (5 ml) and the organics were washed with water (5 ml). The organics were concentrated in vacuo to afford (5R,8S)—N-(5-chloro-2-fluoro-4-(6-(2-(methylamino)ethoxy)pyridin-3-yl)phenyl)-1-fluoro-6,7,8,9-tetrahydro-5H-5,8-epiminocyclohepta[c]pyridine-10-carboxamide 300 as a colourless solid. LCMS (Method 1) m/z 500.3, 502.3 (M+H)⁺ (ES)⁺ at 1.40 min. ¹H NMR (400 MHz, DMSO-d6) δ 8.84 (s, 1H), 8.25-8.13 (m, 1H), 8.01 (d, J=5.0 Hz, 1H), 7.79 (dd, J=8.6, 2.6 Hz, 1H), 7.74 (d, J=7.4 Hz, 1H), 7.37 (d, J=11.1 Hz, 1H), 7.21 (dd, J=5.1, 1.6 Hz, 1H), 6.88 (d, J=8.8 Hz, 1H), 5.22 (d, J=5.9 Hz, 1H), 4.83-4.77 (m, 1H), 4.33 (t, J=5.8 Hz, 2H), 3.26-3.15 (m, 1H), 2.83 (t, J=5.8 Hz, 2H), 2.59 (d, J=17.3 Hz, 1H), 2.32 (s, 3H), 2.30-2.13 (m, 2H), 1.90-1.81 (m, 1H), 1.81-1.71 (m, 1H).

### Experimental Scheme 34

### Compound 128: (6S,9R)—N-(5-Chloro-2-fluoro-4-(5-(hydroxymethyl)pyridin-3-yl)phenyl)-3-oxo-3,5,6,7,8,9-hexahydro-2H-6,9-epiminocyclohepta[c]pyridazine-10-carboxamide

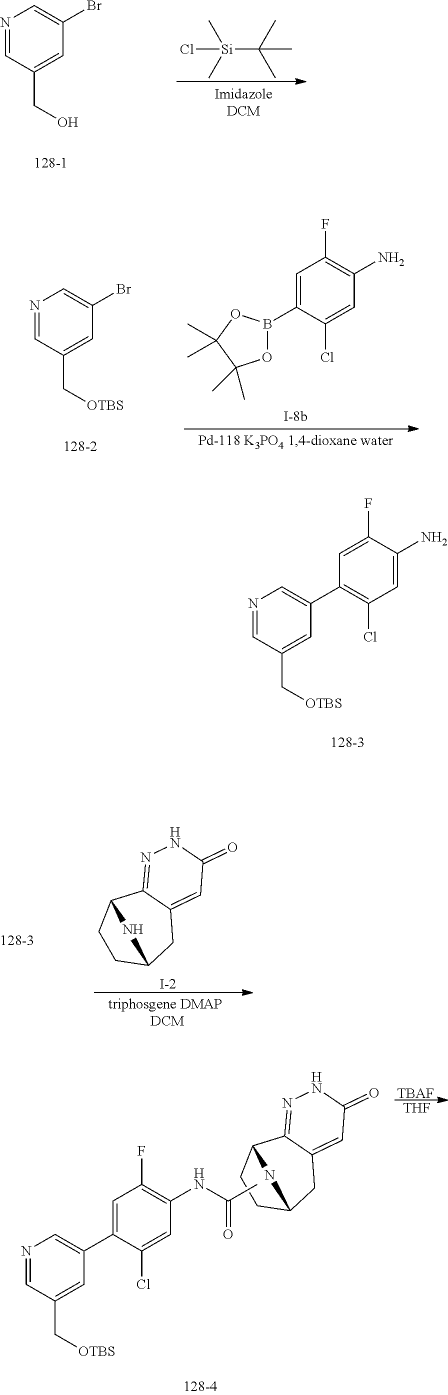

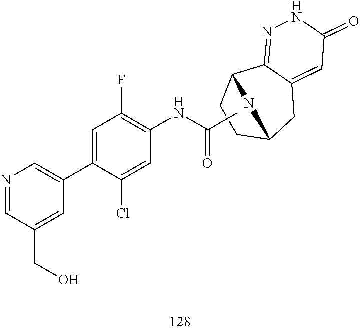

Step 1: To a solution of (3-Bromo-pyridin-5-yl)methanol 128-1 (1.00 g, 5.32 mmol) and imidazole (380 mg, 5.58 mmol) in DCM (50 ml) at 0° C., was added tert-butyldimethylchlorosilane (931 μl, 5.58 mmol) slowly, and the reaction mixture was slowly warmed up to RT and the reaction mixture was stirred at RT for 72 h. An aqueous solution of HCl (25 ml, 1 M) was added. The product was extracted with DCM (10 ml). The combined organics were washed with brine (20 ml), dried over MgSO₄ and concentrated in vacuo to give 3-bromo-5-(((tert-butyldimethylsilyl)oxy)methyl)pyridine 128-2 as a yellowish oil, which was used without any further purification. ¹H NMR (400 MHz, CDCl₃) δ 8.57 (d, J=2.2 Hz, 1H), 8.49-8.45 (m, 1H), 7.84 (tt, J=1.9, 0.8 Hz, 1H), 4.74 (q, J=0.8 Hz, 2H), 0.94 (s, 9H), 0.12 (s, 6H).

Step 2: 4-(5-(((tert-butyldimethylsilyl)oxy)methyl)pyridin-3-yl)-5-chloro-2-fluoroaniline 128-3 was synthesised from 5-chloro-2-fluoro-4-(4,4,5,5-tetramethyl-1,3,2-dioxaborolan-2-yl)aniline I-8b and 3-bromo-5-(((tert-butyldimethylsilyl)oxy)methyl)pyridine 128-2 using a procedure essentially the same as for I-47. ¹H NMR (400 MHz, DMSO-d6) δ 8.47 (dd, J=2.9, 2.1 Hz, 2H), 7.74 (dd, J=2.5, 1.7 Hz, 1H), 7.16 (d, J=11.9 Hz, 1H), 6.93 (d, J=8.3 Hz, 1H), 5.68 (s, 2H), 4.80 (d, J=0.9 Hz, 2H), 0.90 (s, 9H), 0.10 (s, 6H).

Step 3: A solution of 4-(5-(((tert-butyldimethylsilyl)oxy)methyl)pyridin-3-yl)-5-chloro-2-fluoroaniline 128-3 (153 mg, 417 μmol) and DMAP (153 mg, 1.25 mmol) in DCM (3 ml) was added to a solution of triphosgene (50 mg, 167 μmol) in DCM (3 ml) and the resulting mixture was stirred at RT for 15 min. The reaction mixture was added to a suspension of (6S,9R)-2,5,6,7,8,9-hexahydro-3H-6,9-epiminocyclohepta[c]pyridazin-3-one I-2 (74 mg, 417 μmol) in DCM (3 ml) and the resulting solution was left to stir at RT for 18 h. An aqueous solution of HCl (10 ml, 1 M) was added and the layers were separated. The aqueous layer was extracted with DCM/IPA 7:3 (3×10 ml). The combined organics were dried with MgSO₄ and concentrated in vacuo. The product was purified by chromatography on silica gel (0-10% MeOH/DCM) to afford (6S,9R)—N-(4-(5-(((tert-butyldimethylsilyl)oxy)-methyl)pyridin-3-yl)-5-chloro-2-fluorophenyl)-3-oxo-3,5,6,7,8,9-hexahydro-2H-6,9-epiminocyclohepta[c]pyridazine-10-carboxamide 128-4 as a thick yellow oil. ¹H NMR (400 MHz, DMSO-d6) δ 12.81-12.62 (m, 1H), 8.91 (s, 1H), 8.54 (dd, J=11.5, 2.2 Hz, 2H), 7.85-7.77 (m, 2H), 7.42 (d, J=11.0 Hz, 1H), 6.69 (s, 1H), 5.08 (d, J=5.6 Hz, 1H), 4.82 (s, 2H), 4.65 (s, 1H), 3.18 (s, 1H), 2.67-2.61 (m, 1H), 2.26-2.07 (m, 2H), 1.85-1.76 (m, 1H), 1.73-1.66 (m, 1H), 0.90 (s, 9H), 0.10 (s, 6H).

Step 4: To a solution of (6S,9R)—N-(4-(5-(((tert-butyldimethylsilyl)oxy)methyl)pyridin-3-yl)-5-chloro-2-fluorophenyl)-3-oxo-3,5,6,7,8,9-hexahydro-2H-6,9-epiminocyclohepta[c]pyridazine-10-carboxamide 128-4 (131 mg, 230 μmol) in THF (5 ml) at 0° C., was added a solution of TBAF in THF (345 μl, 1 M, 345 μmol) and the reaction mixture was slowly warmed up to RT for 18 h. The reaction mixture was diluted with a saturated aqueous solution of NaHCO₃ (25 ml). The product was extracted with DCM (3×10 ml). The combined organics were dried over MgSO₄ and concentrated in vacuo. The product was purified by chromatography on RP Flash C₁₈ (5-50% MeCN/(0.1% ammonium hydroxide in water)) to afford (6S,9R)—N-(5-chloro-2-fluoro-4-(5-(hydroxymethyl)pyridin-3-yl)phenyl)-3-oxo-3,5,6,7,8,9-hexahydro-2H-6,9-epiminocyclohepta[c]pyridazine-10-carboxamide 128 as a white solid. LCMS (Method 1) m/z 456.3, 458.3 (M+H)⁺ (ES)⁺ at 0.76 min ¹H NMR (400 MHz, DMSO-d6) δ 12.70 (s, 1H), 8.92 (s, 1H), 8.54 (d, J=2.0 Hz, 1H), 8.51 (d, J=2.2 Hz, 1H), 7.82 (d, J=7.3 Hz, 1H), 7.79 (t, J=2.2 Hz, 1H), 7.39 (d, J=11.1 Hz, 1H), 6.68 (s, 1H), 5.39 (t, J=5.6 Hz, 1H), 5.08 (d, J=5.7 Hz, 1H), 4.69-4.61 (m, 1H), 4.60 (d, J=4.6 Hz, 2H), 3.20 (dd, J=18.9, 4.9 Hz, 1H), 2.70-2.60 (m, 1H), 2.30-2.11 (m, 2H), 1.84-1.75 (m, 1H), 1.72-1.63 (m, 1H).

### Experimental Scheme 35

### Compound 311: (6S,9R)—N-(5-Chloro-2-fluoro-4-(5-(fluoromethyl)pyridin-3-yl)phenyl)-3-oxo-3,5,6,7,8,9-hexahydro-2H-6,9-epiminocyclohepta[c]pyridazine-10-carboxamide

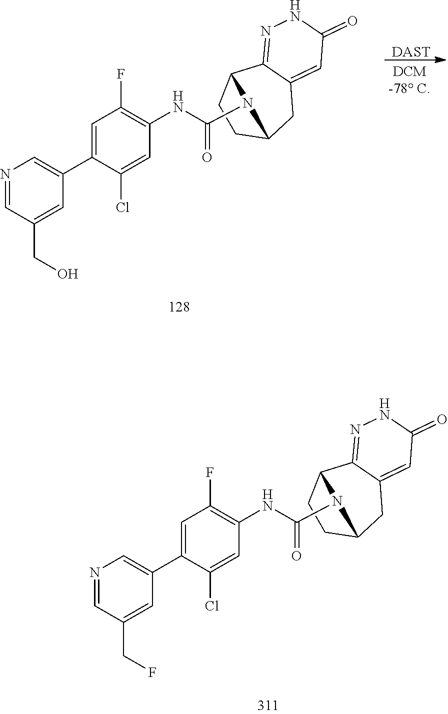

To a suspension of (6S,9R)—N-(5-chloro-2-fluoro-4-(5-(hydroxymethyl)pyridin-3-yl)phenyl)-3-oxo-3,5,6,7,8,9-hexahydro-2H-6,9-epiminocyclohepta[c]pyridazine-10-carboxamide 128 (79.0 mg, 173 μmol) in DCM (5.00 ml) at −78° C. was slowly added DAST (92 μl, 693 μmol). The reaction mixture was slowly warmed to RT and left to stir at RT for 24 h. A saturated aqueous solution of NaHCO₃ (20 ml) was added followed by DCM/IPA 7/3 (60 ml). The mixture was concentrated in vacuo. The product was purified by chromatography on RP Flash C₁₈ (1-95% MeCN/Water (10 mM ammonium bicarbonate) and the product was further purified by chromatography on silica gel (0-10% MeOH/DCM) to afford (6S,9R)—N-(5-chloro-2-fluoro-4-(5-(fluoromethyl)pyridin-3-yl)phenyl)-3-oxo-3,5,6,7,8,9-hexahydro-2H-6,9-epiminocyclohepta[c]pyridazine-10-carboxamide 311 as a white solid. LCMS (Method 1) m/z 458.2, 460.2 (M+H)⁺ (ES)⁺ at 0.98 min. ¹H NMR (400 MHz, DMSO-d6) δ 12.73 (s, 1H), 8.94 (s, 1H), 8.73-8.67 (m, 2H), 8.04-7.99 (m, 1H), 7.83 (d, J=7.2 Hz, 1H), 7.47 (d, J=11.1 Hz, 1H), 6.69 (s, 1H), 5.56 (d, J=47.4 Hz, 2H), 5.09 (d, J=5.7 Hz, 1H), 4.70-4.60 (m, 1H), 3.24-3.15 (m, 1H), 2.66 (d, J=18.0 Hz, 1H), 2.26-2.13 (m, 2H), 1.85-1.77 (m, 1H), 1.73-1.64 (m, 1H).

### Separation of Enantiomers by Chiral Chromatography

It will be appreciated that the enantiomers of the compounds described above can be isolated using techniques well known in the art, including, but not limited to, chiral chromatography. For example, a racemic mixture can be dissolved in a solvent, for example, methanol, followed by separation by chiral SFC on a Waters prep 15 with UV detection by DAD at 210-400 nm, 40° C., 120 bar on a Chiralpak® IG (Daicel Ltd.) column (1×25 cm, 5 μm particle size), flow rate 15 ml/min-1 using 50% ethanol in 0.1% DEA to afford both enantiomers as the separated pure compounds.

For example, (±)—N-(5-chloro-2-fluoro-4-(trifluoromethyl)phenyl)-3-oxo-3,5,6,7,8,9-hexahydro-2H-6,9-epiminocyclohepta[c]pyridazine-10-carboxamide (compound 1) was dissolved to 10 mg/ml in MeOH/DMSO-d6 with sonication, filtered and was then separated by chiral SFC on a Waters prep 15 with UV detection by DAD at 210-400 nm, 40° C., 120 bar. The column was IA 10×250 mm, 5 m, flow rate 15 ml/min at 30% MeOH (0.1% ammonia), 70% CO₂ to afford (6S,9R)—N-(5-chloro-2-fluoro-4-(trifluoromethyl)phenyl)-3-oxo-3,5,6,7,8,9-hexahydro-2H-6,9-epiminocyclohepta[c]pyridazine-10-carboxamide (compound 18) and (6R,9S)—N-(5-chloro-2-fluoro-4-(trifluoromethyl)phenyl)-3-oxo-3,5,6,7,8,9-hexahydro-2H-6,9-epiminocyclohepta[c]pyridazine-10-carboxamide (compound 19) both as colourless solids. LCMS and NMR data for compounds 18 and 19 are found in the table below.

The following compounds were prepared using appropriate starting materials in an analogous procedure to that described for 18 and 19

| | | Method | | |
|---|---|---|---|---|
| | | LCMS | | |
| | | Rt | Analytical | |
| Compound | Structure | [M + H]⁺ | SFC Rt | NMR |
| 18^{(b)(c)} | 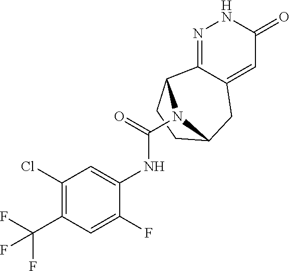 [SMILES: O=C(Nc1cc(Cl)c(C(F)(F)F)cc1F)N1[C@H]2CC[C@@H]1c1n[nH]c(=O)cc1C2] | Method 1 417.1, 419.0 at 1.19 min | 1.87 min, SFC method 1 35% MeOH (0.1% Ammonia), 65% CO₂ | ¹H NMR (400 MHz, DMSO-d6) δ 12.71 (s, 1H), 9.12 (s, 1H), 8.04 (d, J = 6.9 Hz, 1H), 7.76 (d, J = 10.9 Hz, 1H), 6.67 (s, 1H), 5.09 (d, J = 5.5 Hz, 1H), 4.65 (s, 1H), 3.18 (dd, J = 18.0, 5.3 Hz, 1H), 2.64 (d, J = 17.9 Hz, 1H), 2.19 (d, J = 6.8 Hz, 2H), 1.78 (d, J = 11.3 Hz, 1H), |
| | | | | 1.68 (d, J = 11.7 |
| | (6S,9R)-N-(5-Chloro-2-fluoro-4- | | | Hz, 1H |
| | (trifluoromethyl)phenyl)-3-oxo- | | | |
| | 3,5,6,7,8,9-hexahydro-2H-6,9- | | | |
| | epiminocyclohepta[c]pyridazine-10- | | | |
| | carboxamide | | | |
| 19^{(b)(c)} | 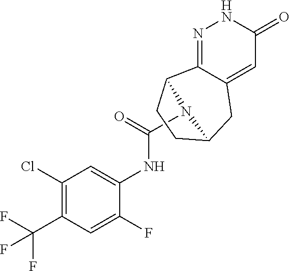 [SMILES: O=C(Nc1cc(Cl)c(C(F)(F)F)cc1F)N1[C@@H]2CC[C@H]1c1n[nH]c(=O)cc1C2] | Method 1 417.1, 419.0 at 1.19 min | 2.36 min, SFC method 1 35% MeOH (0.1% Ammonia), 65% CO₂ | ¹H NMR (400 MHz, DMSO-d6) δ 12.73 (s, 1H), 9.13 (s, 1H), 8.03 (d, J = 6.9 Hz, 1H), 7.79 (d, J = 10.9 Hz, 1H), 6.68 (s, 1H), 5.10 (d, J = 5.6 Hz, 1H), 4.66 (s, 1H), 3.18 (dd, J = 18.4, 5.4 Hz, 1H), 2.65 (d, J = 17.9 Hz, 1H), 2.18 (dd, J = 11.4, 6.3 Hz, 2H), 1.80 (t, J = 9.5 Hz, |
| | | | | 1H), 1.72-1.63 |
| | (6R,9S)-N-(5-Chloro-2-fluoro-4- | | | (m, 1H) |
| | (trifluoromethyl)phenyl)-3-oxo- | | | |
| | 3,5,6,7,8,9-hexahydro-2H-6,9- | | | |
| | epiminocyclohepta[c]pyridazine-10- | | | |
| | carboxamide | | | |
| 20^{(b)(c)} | 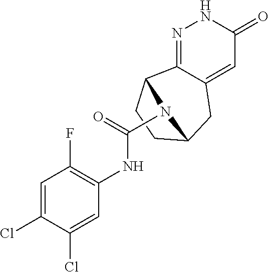 [SMILES: O=C(Nc1cc(Cl)c(Cl)cc1F)N1[C@H]2CC[C@@H]1c1n[nH]c(=O)cc1C2] | Method 1 381.1, 382.8 at 1.05 min(a) | 2.24 min, SFC method 1 45% MeOH (0.1% Ammonia), 55% CO₂ | ¹H NMR (400 MHz, DMSO-d6) δ 12.71 (s, 1H), 8.87 (s, 1H), 7.83 (d, J = 7.5 Hz, 1H), 7.68 (d, J = 10.3 Hz, 1H), 6.68 (s, 1H), 5.05 (d, J = 5.9 Hz, 1H), 4.62 (t, J = 6.1 Hz, 1H), 3.17 (dd, J = 18.6, 5.2 Hz, 1H), 2.64 (d, J = 17.9 Hz, 1H), 2.17 (dd, J = 11.9, 6.4 Hz, 2H), 1.83-1.74 (m, |
| | | | | 1H), 1.71-1.63 |
| | (6S,9R)-N-(4,5-Dichloro-2- | | | (m, 1H) |
| | fluorophenyl)-3-oxo-3,5,6,7,8,9- | | | |
| | hexahydro-2H-6,9- | | | |
| | epiminocyclohepta[c]pyridazine-10- | | | |
| | carboxamide | | | |
| 21^{(b)(c)} | 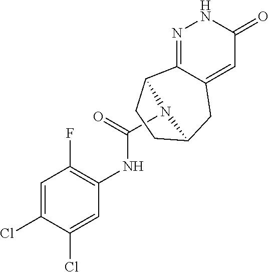 [SMILES: O=C(Nc1cc(Cl)c(Cl)cc1F)N1[C@@H]2CC[C@H]1c1n[nH]c(=O)cc1C2] | Method 1 381.3, 383.3 at 1.05 min(a) | 3.13 min, SFC method 1 45% MeOH (0.1% Ammonia), 55% CO₂ | ¹H NMR (400 MHz, DMSO-d6) δ 12.71 (s, 1H), 8.87 (s, 1H), 7.83 (d, J = 7.5 Hz, 1H), 7.68 (d, J = 10.3 Hz, 1H), 6.68 (s, 1H), 5.05 (d, J = 5.9 Hz, 1H), 4.62 (t, J = 6.1 Hz, 1H), 3.17 (dd, J = 18.6, 5.2 Hz, 1H), 2.64 (d, J = 17.9 Hz, 1H), 2.17 (dd, J = 11.9, 6.4 Hz, 2H), 1.83-1.74 (m, |
| | | | | 1H), 1.71-1.63 |
| | (6R,9S)-N-(4,5-Dichloro-2- | | | (m, 1H) |
| | fluorophenyl)-3-oxo-3,5,6,7,8,9- | | | |
| | hexahydro-2H-6,9- | | | |
| | epiminocyclohepta[c]pyridazine-10- | | | |
| | carboxamide | | | |
| 22^{(d)(e)} | 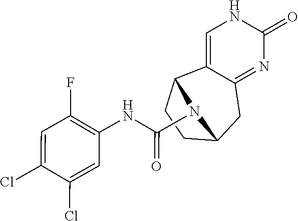 [SMILES: O=C(Nc1cc(Cl)c(Cl)cc1F)N1[C@H]2CC[C@@H]1c1c[nH]c(=O)nc1C2] | Method 2 382.9, 385.0 at 1.08 min | 2.17 min, SFC method 2 45% MeOH (0.1% Ammonia), 55% CO₂ | ¹H NMR (400 MHz, DMSO-d6) δ 7.90 (s, 1H), 7.84 (d, J = 7.6 Hz, 1H), 7.67 (d, J = 10.3 Hz, 1H), 5.04 (d, J = 5.8 Hz, 1H), 4.67- 4.59 (m, 1H), 3.10 (dd, J = 18.6, 4.8 Hz, 1H), 2.26- 2.14 (m, 1H), 2.13-1.99 (m, 1H), 1.83-1.64 (m, |
| | | | | 2H). Two |
| | (5R,8S)-N-(4,5-Dichloro-2- | | | exchangeable |
| | fluorophenyl)-2-oxo-3,5,6,7,8,9- | | | protons not |
| | hexahydro-2H-5,8- | | | observed. One |
| | epiminocyclohepta[d]pyrimidine-10- | | | proton under |
| | carboxamide | | | DMSO-d6 peak |
| | | | | (benzylic |
| | | | | diastereotopic |
| | | | | proton). |
| 23^{(d)(e)} | 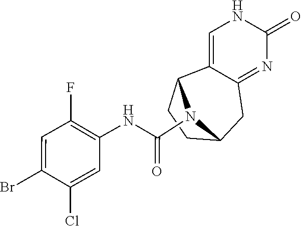 [SMILES: O=C(Nc1cc(Cl)c(Br)cc1F)N1[C@H]2CC[C@@H]1c1c[nH]c(=O)nc1C2] | Method 1 427.4, 429.1 at 0.83 min | 2.38 min SFC method 2 50% MeOH (0.1% Ammonia), 50% CO₂ | ¹H NMR (400 MHz, DMSO-d6) δ 11.66 (s, 1H), 8.74 (s, 1H), 7.84 (d, J = 7.5 Hz, 1H), 7.76 (d, J = 10.1 Hz, 1H), 5.05 (d, J = 5.8 Hz, 1H), 4.64 (t, J = 6.5 Hz, 1H), 3.11 (dd, J = 18.7, 5.0 Hz, 1H), 2.26- 2.15 (m, 1H), 2.11-2.01 (m, |
| | | | | 1H), 1.85-1.65 |
| | (5R,8S)-N-(4-Bromo-5-chloro-2- | | | (m, 2H). (1CH |
| | fluorophenyl)-2-oxo-3,5,6,7,8,9- | | | under DMSO-d6 |
| | hexahydro-2H-5,8- | | | peak) |
| | epiminocyclohepta[d]pyrimidine-10- | | | |
| | carboxamide | | | |
| 24^{(d)(e)} | 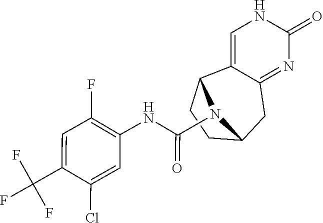 [SMILES: O=C(Nc1cc(Cl)c(C(F)(F)F)cc1F)N1[C@H]2CC[C@@H]1c1c[nH]c(=O)nc1C2] | Method 1 417.1, 419.3 at 0.89 min | 1.92 min SFC method 2 35% MeOH (0.1% Ammonia), 65% CO₂ | ¹H NMR (400 MHz, DMSO-d6) δ 8.05 (d, J = 6.9 Hz, 1H), 7.95 (s, 1H), 7.78 (d, J = 11.0 Hz, 1H), 5.10 (d, J = 5.8 Hz, 1H), 4.72- 4.64 (m, 1H), 3.19-3.08 (m, 1H), 2.28-2.15 (m, 1H), 2.14-2.01 (m, 1H), 1.85- 1.66 (m, 2H). Two |
| | | | | exchangeable |
| | (5R,8S)-N-(5-Chloro-2-fluoro-4- | | | protons not |
| | (trifluoromethyl)phenyl)-2-oxo- | | | observed. One |
| | 3,5,6,7,8,9-hexahydro-2H-5,8- | | | proton obscured |
| | epiminocyclohepta[d]pyrimidine-10- | | | by DMSO-d6 |
| | carboxamide | | | peak. |
| 25^{(d)(e)} | 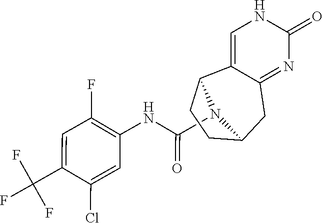 [SMILES: O=C(Nc1cc(Cl)c(C(F)(F)F)cc1F)N1[C@@H]2CC[C@H]1c1c[nH]c(=O)nc1C2] | Method 1 417.2, 419.1 at 0.89 min | 3.50 min SFC method 2 35% MeOH (0.1% Ammonia), 65% CO₂ | ¹H NMR (400 MHz, DMSO-d6) δ 8.05 (d, J = 6.9 Hz, 1H), 7.95 (s, 1H), 7.77 (d, J = 11.0 Hz, 1H), 5.10 (d, J = 5.8 Hz, 1H), 4.72- 4.64 (m, 1H), 3.18-3.08 (m, 1H), 2.27-2.15 (m, 1H), 2.14-2.01 (m, 1H), 1.85- 1.66 (m, 2H). Two |
| | | | | exchangeable |
| | (5S,8R)-N-(5-Chloro-2-fluoro-4- | | | protons not |
| | (trifluoromethyl)phenyl)-2-oxo- | | | observed. One |
| | 3,5,6,7,8,9-hexahydro-2H-5,8- | | | proton obscured |
| | epiminocyclohepta[d]pyrimidine-10- | | | by DMSO-d6 |
| | carboxamide | | | peak. |
| 26^{(b)(e)} | 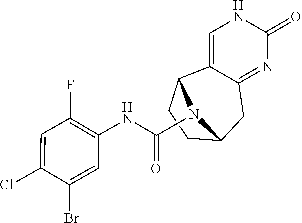 [SMILES: O=C(Nc1cc(Br)c(Cl)cc1F)N1[C@H]2CC[C@@H]1c1c[nH]c(=O)nc1C2] | Method 1 427.2, 429.4, 431.0 at 0.79 min | 1.93 min SFC method 2 45% MeOH (0.1% Ammonia), 55% CO₂ | ¹H NMR (400 MHz, DMSO-d6) δ 7.20-7.02 (m, 2H), 6.65-6.51 (m, 1H), 4.37- 4.24 (m, 1H), 3.97-3.84 (m, 1H), 2.68 (s, 1H), 1.91- 1.78 (m, 1H), 1.67-1.36 (m, 2H), 1.20-0.95 (m, 2H). Exchangeable protons not observed |
| | (5R,8S)-N-(5-Bromo-4-chloro-2- | | | |
| | fluorophenyl)-2-oxo-3,5,6,7,8,9- | | | |
| | hexahydro-2H-5,8- | | | |
| | epiminocyclohepta[d]pyrimidine-10- | | | |
| | carboxamide | | | |
| 27^{(b)(e)} | 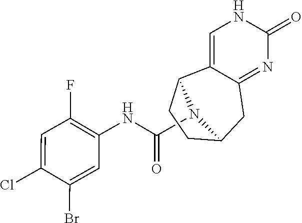 [SMILES: O=C(Nc1cc(Br)c(Cl)cc1F)N1[C@@H]2CC[C@H]1c1c[nH]c(=O)nc1C2] | Method 1 427.3, 429.4, 431.0 at 0.79 min | 3.09 min SFC method 2 45% MeOH (0.1% Ammonia), 55% CO₂ | ¹H NMR (400 MHz, DMSO-d6) δ 7.20-7.02 (m, 2H), 6.65-6.51 (m, 1H), 4.37- 4.24 (m, 1H), 3.97-3.84 (m, 1H), 2.68 (s, 1H), 1.91- 1.78 (m, 1H), 1.67-1.36 (m, 2H), 1.20-0.95 (m, 2H). Exchangeable protons not observed |
| | (5S,8R)-N-(5-Bromo-4-chloro-2- | | | |
| | fluorophenyl)-2-oxo-3,5,6,7,8,9- | | | |
| | hexahydro-2H-5,8- | | | |
| | epiminocyclohepta[d]pyrimidine-10- | | | |
| | carboxamide | | | |
| 28^{(f)(g)} | 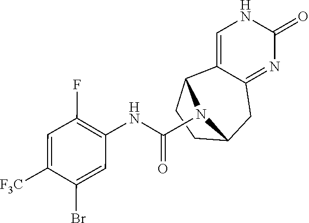 [SMILES: O=C(Nc1cc(Br)c(C(F)(F)F)cc1F)N1[C@H]2CC[C@@H]1c1c[nH]c(=O)nc1C2] | Method 1 460.6, 463.4 at 0.86 min | 3.84 min SFC method 1 30% MeOH (0.1% Ammonia), 70% CO₂ | ¹H NMR (400 MHz, DMSO-d6) δ 8.21 (d, J = 7.0 Hz, 1H), 7.93 (s, 1H), 7.74 (d, J = 10.7 Hz, 1H), 5.08 (d, J = 5.7 Hz, 1H), 4.67 (s, 1H), 3.10 (s, 1H), 2.54 (s, 1H), 2.20 (d, J = 11.3 Hz, 1H), 2.14-2.00 (m, 1H), 1.75 (dt, J = 29.4, 9.0 Hz, |
| | | | | 2H). |
| | (5R,8S)-N-(5-Bromo-2-fluoro-4- | | | Exchangeable |
| | (trifluoromethyl)phenyl)-2-oxo- | | | protons not |
| | 3,5,6,7,8,9-hexahydro-2H-5,8- | | | observed, 1 CH |
| | epiminocyclohepta[d]pyrimidine-10- | | | under DMSO |
| | carboxamide | | | peak |
| 29^{(f)(g)} | 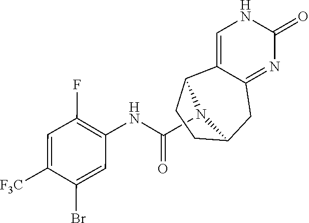 [SMILES: O=C(Nc1cc(Br)c(C(F)(F)F)cc1F)N1[C@@H]2CC[C@H]1c1c[nH]c(=O)nc1C2] | Method 1 460.6, 463.0 at 0.87 min | 4.57 min SFC method 1 30% MeOH (0.1% Ammonia), 70% CO₂ | ¹H NMR (400 MHz, DMSO-d6) δ 8.21 (d, J = 7.2 Hz, 1H), 7.94 (s, 1H), 7.75 (d, J = 11.2 Hz, 1H), 5.09 (d, J = 5.8 Hz, 1H), 4.67 (t, J = 6.5 Hz, 1H), 3.13 (dd, J = 18.4, 5.0 Hz, 1H), 2.21 (q, J = 11.0 Hz, 1H), 2.07 (tt, J = 11.4, 6.0 Hz, 1H), |
| | | | | 1.84-1.65 (m, |
| | (5S,8R)-N-(5-Bromo-2-fluoro-4- | | | 2H). |
| | (trifluoromethyl)phenyl)-2-oxo- | | | Exchangeable |
| | 3,5,6,7,8,9-hexahydro-2H-5,8- | | | protons not |
| | epiminocyclohepta[d]pyrimidine-10- | | | observed, 1 CH |
| | carboxamide | | | under DMSO-d6 |
| | | | | peak |
| 30^{(d)(h)} | 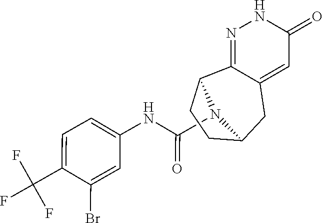 [SMILES: O=C(Nc1ccc(C(F)(F)F)c(Br)c1)N1[C@@H]2CC[C@H]1c1n[nH]c(=O)cc1C2] | Method 1 (ES⁻) 441.0, 442.1, 443.1 at 1.20 min | 3.07 min SFC method 3 30% MeOH (0.1% Ammonia), 70% CO₂ | ¹H NMR (400 MHz, DMSO-d6) δ 8.08 (d, J = 1.9 Hz, 1H), 7.74- 7.62 (m, 2H), 6.71-6.64 (m, 1H), 5.13 (d, J = 5.9 Hz, 1H), 4.74- 4.61 (m, 1H), 3.15 (dd, J = 18.4, 5.3 Hz, 1H), 2.66 (d, J = 18.2 Hz, 1H), 2.28-2.07 (m, 2H), 1.86-1.75 |
| | | | | (m, 1H), 1.75- |
| | (6R,9S)-N-(3-Bromo-4- | | | 1.63 (m, 1H). Two |
| | (trifluoromethyl)phenyl)-3-oxo- | | | exchangeable |
| | 3,5,6,7,8,9-hexahydro-2H-6,9- | | | protons not |
| | epiminocyclohepta[c]pyridazine-10- | | | observed. |
| | carboxamide | | | |
| 31^{(d)(h)} | 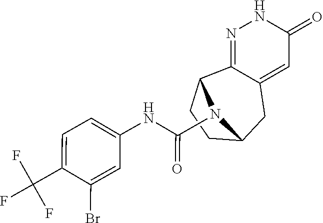 [SMILES: O=C(Nc1ccc(C(F)(F)F)c(Br)c1)N1[C@H]2CC[C@@H]1c1n[nH]c(=O)cc1C2] | Method 1 (ES⁻) 441.0, 442.1, 443.1 at 1.20 min | 3.98 min SFC method 3 30% MeOH (0.1% Ammonia), 70% CO₂ | ¹H NMR (400 MHz, DMSO-d6) δ 8.08 (d, J = 1.9 Hz, 1H), 7.74- 7.62 (m, 2H), 6.71-6.64 (m, 1H), 5.13 (d, J = 5.9 Hz, 1H), 4.74- 4.61 (m, 1H), 3.15 (dd, J = 18.4, 5.3 Hz, 1H), 2.66 (d, J = 18.2 Hz, 1H), 2.28-2.07 (m, 2H), 1.86-1.75 |
| | | | | (m, 1H), 1.75- |
| | (6S,9R)-N-(3-Bromo-4- | | | 1.63 (m, 1H). Two |
| | (trifluoromethyl)phenyl)-3-oxo- | | | exchangeable |
| | 3,5,6,7,8,9-hexahydro-2H-6,9- | | | protons not |
| | epiminocyclohepta[c]pyridazine-10- | | | observed. |
| | carboxamide | | | |
| 32^{(d)(c)} | 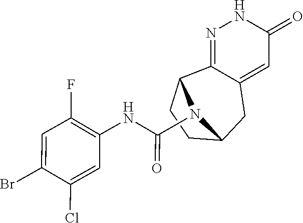 [SMILES: O=C(Nc1cc(Cl)c(Br)cc1F)N1[C@H]2CC[C@@H]1c1n[nH]c(=O)cc1C2] | Method 1 (ES⁻) 425.1, 427.0 at 1.10 min | 3.28 min SFC method 1 40% MeOH (0.1% Ammonia), 60% CO₂ | ¹H NMR (400 MHz, DMSO-d6) δ 7.83 (d, J = 7.5 Hz, 1H), 7.75 (d, J = 10.1 Hz, 1H), 6.67 (s, 1H), 5.05 (d, J = 5.8 Hz, 1H), 4.65-4.56 (m, 1H), 3.22- 3.09 (m, 1H), 2.64 (d, J = 18.4 Hz, 1H), 2.25-2.09 (m, 2H), 1.83- 1.73 (m, 1H), |
| | | | | 1.72-1.62 (m, 1H). |
| | (6S,9R)-N-(4-Bromo-5-chloro-2- | | | Two |
| | fluorophenyl)-3-oxo-3,5,6,7,8,9- | | | exchangeable |
| | hexahydro-2H-6,9- | | | protons not |
| | epiminocyclohepta[c]pyridazine-10- | | | observed. |
| | carboxamide | | | |
| 33^{(d)(c)} | 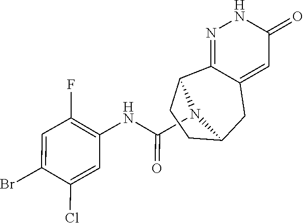 [SMILES: O=C(Nc1cc(Cl)c(Br)cc1F)N1[C@@H]2CC[C@H]1c1n[nH]c(=O)cc1C2] | Method 1 (ES⁻) 425.1, 427.1 at 1.13 min | 4.88 min SFC method 1 40% MeOH (0.1% Ammonia), 60% CO₂ | ¹H NMR (400 MHz, DMSO-d6) δ 7.83 (d, J = 7.5 Hz, 1H), 7.75 (d, J = 10.1 Hz, 1H), 6.67 (s, 1H), 5.05 (d, J = 5.8 Hz, 1H), 4.66-4.56 (m, 1H), 3.23- 3.10 (m, 1H), 2.64 (d, J = 17.9 Hz, 1H), 2.27-2.08 (m, 2H), 1.85- 1.74 (m, 1H), |
| | | | | 1.72-1.59 (m, 1H). |
| | (6R,9S)-N-(4-Bromo-5-chloro-2- | | | Two |
| | fluorophenyl)-3-oxo-3,5,6,7,8,9- | | | exchangeable |
| | hexahydro-2H-6,9- | | | protons not |
| | epiminocyclohepta[c]pyridazine-10- | | | observed. |
| | carboxamide | | | |
| 34^{(d)(h)} | 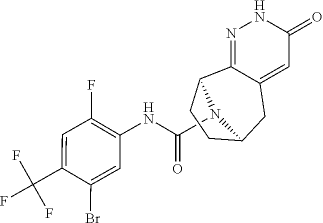 [SMILES: O=C(Nc1cc(Br)c(C(F)(F)F)cc1F)N1[C@@H]2CC[C@H]1c1n[nH]c(=O)cc1C2] | Method 1 (ES⁻) 459.3, 460.8 at 1.22 min | 3.07 min SFC method 3 30% MeOH (0.1% Ammonia), 70% CO₂ | ¹H NMR (400 MHz, DMSO-d6) δ 12.72 (s, 1H), 9.12 (s, 1H), 8.19 (d, J = 7.2 Hz, 1H), 7.75 (d, J = 11.2 Hz, 1H), 6.67 (s, 1H), 5.09 (d, J = 5.6 Hz, 1H), 4.73-4.56 (m, 1H), 3.18 (dd, J = 18.3, 5.2 Hz, 1H), 2.65 (d, J = 18.2 Hz, 1H), |
| | | | | 2.28-2.09 (m, |
| | (6R,9S)-N-(5-Bromo-2-fluoro-4- | | | 2H), 1.87-1.75 |
| | (trifluoromethyl)phenyl)-3-oxo- | | | (m, 1H), 1.73- |
| | 3,5,6,7,8,9-hexahydro-2H-6,9- | | | 1.60 (m, 1H). |
| | epiminocyclohepta[c]pyridazine-10- | | | |
| | carboxamide | | | |
| 35^{(d)(h)} | 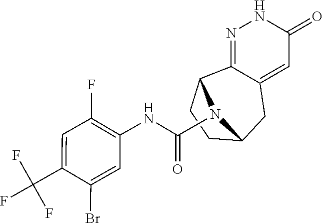 [SMILES: O=C(Nc1cc(Br)c(C(F)(F)F)cc1F)N1[C@H]2CC[C@@H]1c1n[nH]c(=O)cc1C2] | Method 1 (ES⁻) 459.2, 461.1 at 1.22 min | 3.96 min SFC method 3 30% MeOH (0.1% Ammonia), 70% CO2 | ¹H NMR (400 MHz, DMSO-d6) δ 12.72 (s, 1H), 9.12 (s, 1H), 8.19 (d, J = 7.2 Hz, 1H), 7.75 (d, J = 11.2 Hz, 1H), 6.67 (s, 1H), 5.09 (d, J = 5.6 Hz, 1H), 4.75-4.52 (m, 1H), 3.18 (dd, J = 18.2, 5.3 Hz, 1H), 2.64 (d, J = 18.5 Hz, 1H), |
| | | | | 2.28-2.08 (m, |
| | (6S,9R)-N-(5-Bromo-2-fluoro-4- | | | 2H), 1.87-1.74 |
| | (trifluoromethyl)phenyl)-3-oxo- | | | (m, 1H), 1.74- |
| | 3,5,6,7,8,9-hexahydro-2H-6,9- | | | 1.61 (m, 1H). |
| | epiminocyclohepta[c]pyridazine-10- | | | |
| | carboxamide | | | |
| 37^{(d)(c)} | 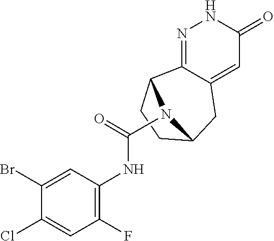 [SMILES: O=C(Nc1cc(Br)c(Cl)cc1F)N1[C@H]2CC[C@@H]1c1n[nH]c(=O)cc1C2] | Method 1 426.7, 428.9, 429.3 at 1.1 min | 3.17 min SFC method 1 40% MeOH (0.1% Ammonia), 60% CO₂ | ¹H NMR (400 MHz, DMSO-d6) δ 12.71 (s, 1H), 8.85 (s, 1H), 7.95 (d, J = 7.8 Hz, 1H), 7.65 (d, J = 10.4 Hz, 1H), 6.67 (s, 1H), 5.04 (d, J = 5.8 Hz, 1H), 4.61 (s, 1H), 3.22-3.12 (m, 1H), 2.63 (d, J = 18.2 Hz, 1H), 2.16 (dd, J = 11.8, 6.4 Hz, 2H), 1.78 (t, J = 9.3 Hz, 1H), |
| | | | | 1.67 (d, J = 11.0 |
| | (6S,9R)-N-(5-Bromo-4-chloro-2- | | | Hz, 1H). |
| | fluorophenyl)-3-oxo-3,5,6,7,8,9- | | | |
| | hexahydro-2H-6,9- | | | |
| | epiminocyclohepta[c]pyridazine-10- | | | |
| | carboxamide | | | |
| 38^{(d)(c)} | 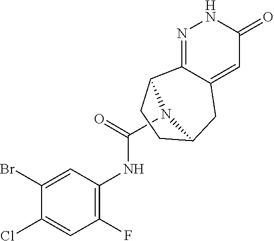 [SMILES: O=C(Nc1cc(Br)c(Cl)cc1F)N1[C@@H]2CC[C@H]1c1n[nH]c(=O)cc1C2] | Method 1 427.1, 429.0 at 1.1 min | 4.72 min SFC method 1 40% MeOH (0.1% Ammonia), 60% CO₂ | ¹H NMR (400 MHz, DMSO-d6) δ 12.71 (s, 1H), 8.86 (s, 1H), 7.95 (d, J = 7.8 Hz, 1H), 7.65 (d, J = 10.4 Hz, 1H), 6.67 (s, 1H), 5.04 (d, J = 6.0 Hz, 1H), 4.62 (d, J = 6.2 Hz, 1H), 3.17 (dd, J = 18.0, 5.4 Hz, 1H), 2.63 (d, J = 18.3 Hz, 1H), 2.16 (dd, J = 11.9, |
| | | | | 6.4 Hz, 2H), 1.78 |
| | (6R,9S)-N-(5-Bromo-4-chloro-2- | | | (t, J = 9.5 Hz, 1H), |
| | fluorophenyl)-3-oxo-3,5,6,7,8,9- | | | 1.66 (s, 1H). |
| | hexahydro-2H-6,9- | | | |
| | epiminocyclohepta[c]pyridazine-10- | | | |
| | carboxamide | | | |
| 39^{(d)(c)} | 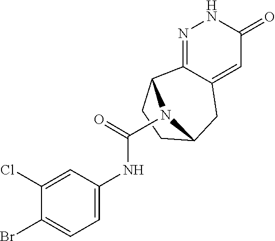 [SMILES: O=C(Nc1ccc(Br)c(Cl)c1)N1[C@H]2CC[C@@H]1c1n[nH]c(=O)cc1C2] | Method 1 408.9, 411.2 at 1.13 min | 3.60 min SFC method 1 35% MeOH (0.1% Ammonia), 65% CO₂ | ¹H NMR (400 MHz, DMSO-d6) δ 12.73 (s, 1H), 9.05 (s, 1H), 7.85 (d, J = 2.5 Hz, 1H), 7.60 (d, J = 8.8 Hz, 1H), 7.39 (dd, J = 8.9, 2.5 Hz, 1H), 6.67 (s, 1H), 5.10 (d, J = 5.7 Hz, 1H), 4.65 (d, J = 6.3 Hz, 1H), 3.14 (dd, J = 18.5, 5.3 Hz, 1H), 2.16 (dd, J = 12.3, |
| | | | | 6.4 Hz, 2H), 1.79 |
| | (6S,9R)-N-(4-Bromo-3- | | | (t, J = 9.8 Hz, 1H), |
| | chlorophenyl)-3-oxo-3,5,6,7,8,9- | | | 1.66 (s, 1H). |
| | hexahydro-2H-6,9- | | | |
| | epiminocyclohepta[c]pyridazine-10- | | | |
| | carboxamide | | | |
| 40^{(d)(c)} | 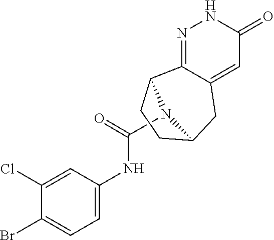 [SMILES: O=C(Nc1ccc(Br)c(Cl)c1)N1[C@@H]2CC[C@H]1c1n[nH]c(=O)cc1C2] | Method 1 408.9, 411.2 at 1.13 min | 5.00 min SFC method 1 35% MeOH (0.1% Ammonia), 65% CO₂ | ¹H NMR (400 MHz, DMSO-d6) δ 12.73 (s, 1H), 9.04 (s, 1H), 7.85 (d, J = 2.5 Hz, 1H), 7.60 (d, J = 8.9 Hz, 1H), 7.39 (dd, J = 8.8, 2.5 Hz, 1H), 6.67 (s, 1H), 5.10 (d, J = 6.0 Hz, 1H), 4.65 (d, J = 6.2 Hz, 1H), 3.14 (d, J = 15.8 Hz, 1H), 2.62 (s, 1H), 2.23- |
| | | | | 2.09 (m, 2H), |
| | (6R,9S)-N-(4-Bromo-3- | | | 1.79 (t, J = 9.7 Hz, |
| | chlorophenyl)-3-oxo-3,5,6,7,8,9- | | | 1H), 1.67 (d, J = |
| | hexahydro-2H-6,9- | | | 11.9 Hz, 1H). |
| | epiminocyclohepta[c]pyridazine-10- | | | |
| | carboxamide | | | |
| 41^{(d)(c)} | 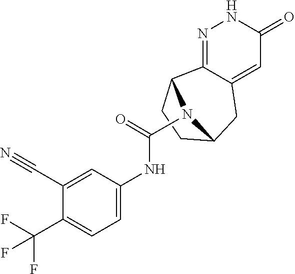 [SMILES: N#Cc1cc(NC(=O)N2[C@H]3CC[C@@H]2c2n[nH]c(=O)cc2C3)ccc1C(F)(F)F] | Method 1 390.2 at 1.06 min | 2.34 min SFC method 1 30% MeOH (0.1% Ammonia), 70% CO₂ | ¹H NMR (400 MHz, DMSO-d6) δ 12.73 (s, 1H), 9.47 (s, 1H), 8.22 (d, J = 2.2 Hz, 1H), 7.95 (dd, J = 8.7, 2.2 Hz, 1H), 7.87 (d, J = 8.9 Hz, 1H), 6.67 (d, J = 1.7 Hz, 1H), 5.15 (d, J = 5.8 Hz, 1H), 4.69 (t, J = 6.2 Hz, 1H), 3.16 (dd, J = 18.3, 5.4 Hz, 1H), 2.72- 2.63 (m, 1H), 2.28-2.09 (m, |
| | | | | 2H), 1.89-1.77 |
| | (6S,9R)-N-(3-Cyano-4- | | | (m, 1H), 1.74- |
| | (trifluoromethyl)phenyl)-3-oxo- | | | 1.61 (m, 1H). |
| | 3,5,6,7,8,9-hexahydro-2H-6,9- | | | |
| | epiminocyclohepta[c]pyridazine-10- | | | |
| | carboxamide | | | |
| 45^{(d)(i)} | 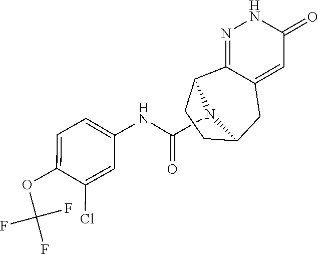 [SMILES: O=C(Nc1ccc(OC(F)(F)F)c(Cl)c1)N1[C@@H]2CC[C@H]1c1n[nH]c(=O)cc1C2] | Method 1 415.3 at 1.63 min | 2.34 min SFC method 4 25% MeOH (0.1% Ammonia), 75% CO₂ | ¹H NMR (400 MHz, DMSO-d6) δ 12.73 (s, 1H), 9.12 (s, 1H), 7.87 (d, J = 2.5 Hz, 1H), 7.52 (dd, J = 9.1, 2.5 Hz, 1H), 7.44 (dd, J = 9.0, 1.4 Hz, 1H), 6.67 (s, 1H), 5.11 (d, J = 5.9 Hz, 1H), 4.66 (d, J = 6.4 Hz, 1H), 3.20- 3.11 (m, 1H), 2.65 (d, J = 17.9 Hz, 1H), 2.24-2.12 |
| | | | | (m, 2H), 1.84- |
| | (6R,9S)-N-(3-Chloro-4- | | | 1.75 (m, 1H), |
| | (trifluoromethoxy)phenyl)-3-oxo- | | | 1.73-1.62 (m, 1H). |
| | 3,5,6,7,8,9-hexahydro-2H-6,9- | | | |
| | epiminocyclohepta[c]pyridazine-10- | | | |
| | carboxamide | | | |
| 46^{(d)(i)} | 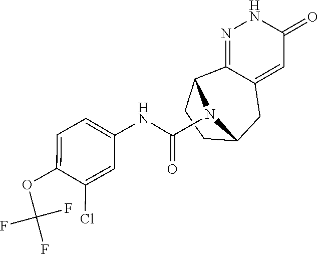 [SMILES: O=C(Nc1ccc(OC(F)(F)F)c(Cl)c1)N1[C@H]2CC[C@@H]1c1n[nH]c(=O)cc1C2] | Method 1 415.3 at 1.63 min | 4.89 min SFC method 4 25% MeOH (0.1% Ammonia), 75% CO₂ | ¹H NMR (400 MHz, DMSO-d6) δ 12.70 (s, 1H), 9.13 (s, 1H), 7.87 (d, J = 2.5 Hz, 1H), 7.52 (dd, J = 9.1, 2.6 Hz, 1H), 7.44 (dd, J = 9.0, 1.4 Hz, 1H), 6.66 (s, 1H), 5.11 (d, J = 5.9 Hz, 1H), 4.66 (d, J = 6.3 Hz, 1H), 3.15 (dd, J = 18.3, 5.3 Hz, 1H), 2.65 (d, J = 18.1 Hz, 1H), |
| | | | | 2.27-2.09 (m, |
| | (6S,9R)-N-(3-Chloro-4- | | | 2H), 1.80 (t, J = |
| | (trifluoromethoxy)phenyl)-3-oxo- | | | 9.8 Hz, 1H), 1.72- |
| | 3,5,6,7,8,9-hexahydro-2H-6,9- | | | 1.63 (m, 1H) |
| | epiminocyclohepta[c]pyridazine-10- | | | |
| | carboxamide | | | |
| 47^{(b)(h)} | 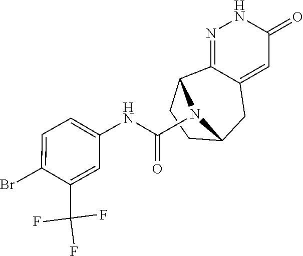 [SMILES: O=C(Nc1ccc(Br)c(C(F)(F)F)c1)N1[C@H]2CC[C@@H]1c1n[nH]c(=O)cc1C2] | Method 1 443.1, 445.3 at 1.25 min | 2.04 min SFC method 3 40% MeOH (0.1% Ammonia), 60% CO₂ | ¹H NMR (400 MHz, DMSO-d6) δ 12.73 (s, 1H), 9.18 (s, 1H), 8.09- 8.04 (m, 1H), 7.77-7.68 (m, 2H), 6.67 (s, 1H), 5.12 (d, J = 5.9 Hz, 1H), 4.67 (d, J = 6.4 Hz, 1H), 3.15 (dd, J = 18.3, 5.4 Hz, 1H), 2.66 (d, J = 18.2 Hz, 1H), 2.24-2.09 (m, 2H), 1.85- 1.75 (m, 1H), 1.72-1.63 (m, 1H). |
| | (6S,9R)-N-(4-Bromo-3- | | | |
| | (trifluoromethyl)phenyl)-3-oxo- | | | |
| | 3,5,6,7,8,9-hexahydro-2H-6,9- | | | |
| | epiminocyclohepta[c]pyridazine-10- | | | |
| | carboxamide | | | |
| 48^{(b)(h)} | 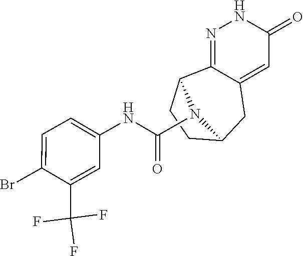 [SMILES: O=C(Nc1ccc(Br)c(C(F)(F)F)c1)N1[C@@H]2CC[C@H]1c1n[nH]c(=O)cc1C2] | Method 1 443.1, 445.1 at 1.25 min | 2.51 min SFC method 3 40% MeOH (0.1% Ammonia), 60% CO₂ | ¹H NMR (400 MHz, DMSO-d6) δ 12.73 (s, 1H), 9.18 (s, 1H), 8.06 (d, J = 1.8 Hz, 1H), 7.73 (d, J = 1.4 Hz, 2H), 6.67 (s, 1H), 5.12 (d, J = 5.9 Hz, 1H), 4.70-4.64 (m, 1H), 3.15 (dd, J = 18.1, 5.3 Hz, 1H), 2.66 (d, J = 18.0 Hz, 1H), 2.27- 2.03 (m, 2H), 1.85-1.75 (m, 1H), |
| | | | | 1.68 (d, J = 10.5 |
| | (6R,9S)-N-(4-Bromo-3- | | | Hz, 1H). |
| | (trifluoromethyl)phenyl)-3-oxo- | | | |
| | 3,5,6,7,8,9-hexahydro-2H-6,9- | | | |
| | epiminocyclohepta[c]pyridazine-10- | | | |
| | carboxamide | | | |
| 49^{(d)(e)} | 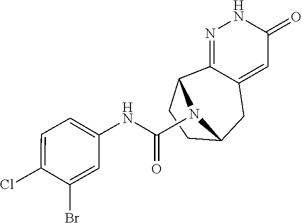 [SMILES: O=C(Nc1ccc(Cl)c(Br)c1)N1[C@H]2CC[C@@H]1c1n[nH]c(=O)cc1C2] | Method 1 409.0, 411.2, 413.2 at 1.14 min | 3.99 min SFC method 2 40% MeOH (0.1% Ammonia), 60% CO₂ | ¹H NMR (400 MHz, DMSO-d6) δ 12.73 (s, 1H), 9.02 (s, 1H), 7.98 (d, J = 2.3 Hz, 1H), 7.51 (dd, J = 8.9, 2.3 Hz, 1H), 7.47 (d, J = 8.8 Hz, 1H), 6.67 (s, 1H), 5.10 (d, J = 5.9 Hz, 1H), 4.64 (t, J = 6.2 Hz, 1H), 3.14 (dd, J = 20.5, 5.2 Hz, 1H), 2.65 |
| | | | | (d, J = 18.2 Hz, |
| | (6S,9R)-N-(3-Bromo-4- | | | 1H), 2.26-2.06 |
| | chlorophenyl)-3-oxo-3,5,6,7,8,9- | | | (m, 2H), 1.84- |
| | hexahydro-2H-6,9- | | | 1.74 (m, 1H), |
| | epiminocyclohepta[c]pyridazine-10- | | | 1.73-1.59 (m, 1H). |
| | carboxamide | | | |
| 50^{(d)(e)} | 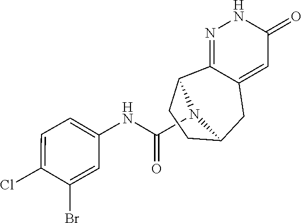 [SMILES: O=C(Nc1ccc(Cl)c(Br)c1)N1[C@@H]2CC[C@H]1c1n[nH]c(=O)cc1C2] | Method 1 409.2, 411.1, 413.1 at 1.14 min | 4.32 min SFC method 2 40% MeOH (0.1% Ammonia), 60% CO₂ | ¹H NMR (400 MHz, DMSO-d6) δ 12.73 (s, 1H), 9.03 (s, 1H), 7.98 (d, J = 2.3 Hz, 1H), 7.51 (dd, J = 8.8, 2.3 Hz, 1H), 7.47 (d, J = 8.8 Hz, 1H), 6.67 (s, 1H), 5.10 (d, J = 5.9 Hz, 1H), 4.64 (t, J = 6.1 Hz, 1H), 3.18-3.09 (m, 1H), 2.64 (d, J = 18.2 Hz, 1H), |
| | | | | 2.26-2.08 (m, |
| | (6R,9S)-N-(3-Bromo-4- | | | 2H), 1.84-1.74 |
| | chlorophenyl)-3-oxo-3,5,6,7,8,9- | | | (m, 1H), 1.71- |
| | hexahydro-2H-6,9- | | | 1.62 (m, 1H). |
| | epiminocyclohepta[c]pyridazine-10- | | | |
| | carboxamide | | | |
| 193^{(b)(e)} | 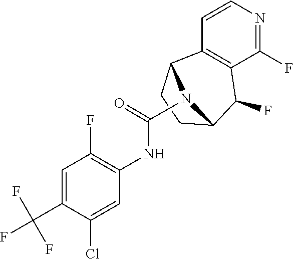 [SMILES: O=C(Nc1cc(Cl)c(C(F)(F)F)cc1F)N1[C@@H]2CC[C@H]1[C@H](F)c1c2ccnc1F] | Method 2 434.2, 436.2 at 1.71 min^{(a)} | 1.36 min SFC method 2 35% MeOH (0.1% Ammonia), 65% CO₂ | ¹H NMR (400 MHz, DMSO-d6) δ 9.15 (s, 1H), 8.33-8.17 (m, 1H), 8.01 (d, J = 6.9 Hz, 1H), 7.80 (d, J = 11.0 Hz, 1H), 7.40 (d, J = 5.1 Hz, 1H), 5.55 (d, J = 49.5 Hz, 1H), 5.35 (d, J = 6.3 Hz, 1H), 5.23 (t, J = 10.0 Hz, 1H), 2.30-2.16 (m, 1H), 2.10 (s, 1H), 1.57 (d, J = 8.3 Hz, 2H) |
| | (5R,8S,9R)-N-(5-Chloro-2-fluoro-4- | | | |
| | (trifluoromethyl)phenyl)-1,9- | | | |
| | difluoro-6,7,8,9-tetrahydro-5H-5,8- | | | |
| | epiminocyclohepta[c]pyridine-10- | | | |
| | carboxamide | | | |
| 227^{(f)(i)} | 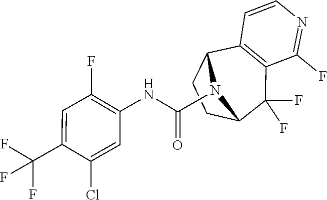 [SMILES: O=C(Nc1cc(Cl)c(C(F)(F)F)cc1F)N1[C@@H]2CC[C@H]1C(F)(F)c1c2ccnc1F] | Methods 1 452.1, 454.2 at 1.63 min^{(a)} | 1.74 min SFC method 5 20% MeOH (0.1% Ammonia), 80% CO₂ | ¹H NMR (400 MHz, DMSO-d6) δ 9.40 (s, 1H), 8.39 (d, J = 5.1 Hz, 1H), 7.99 (d, J = 6.9 Hz, 1H), 7.83 (d, J = 11.0 Hz, 1H), 7.57- 7.45 (m, 1H), 5.47 (d, J = 6.5 Hz, 1H), 5.26 (dd, J = 12.0, 8.0 Hz, 1H), 2.34 (q, J = 11.1 |
| | | | | Hz, 1H), 2.19 (tt, |
| | (5R,8S)-N-(5-Chloro-2-fluoro-4- | | | J = 12.2, 6.6 Hz, |
| | (trifluoromethyl)phenyl)-1,9,9- | | | 1H), 1.92 (dd, J = |
| | trifluoro-6,7,8,9-tetrahydro-5H-5,8- | | | 14.9, 8.3 Hz, 1H), |
| | epiminocyclohepta[c]pyridine-10- | | | 1.72 (ddd, J = |
| | carboxamide | | | 12.1, 9.1, 2.6 Hz, |
| | | | | 1H). |
| 228^{(f)(i)} | 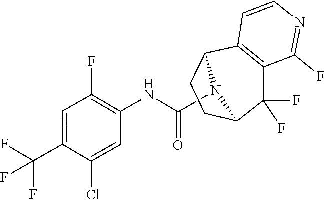 [SMILES: O=C(Nc1cc(Cl)c(C(F)(F)F)cc1F)N1[C@H]2CC[C@@H]1C(F)(F)c1c2ccnc1F] | Method 2 451.9, 454.2 at 1.63 min^{(a)} | 1.39 min SFC method 5 20% MeOH (0.1% Ammonia), 80% CO₂ | ¹H NMR (400 MHz, DMSO-d6) 0 9.40 (s, 1H), 8.39 (d, J = 5.1 Hz, 1H), 7.99 (d, J = 6.9 Hz, 1H), 7.83 (d, J = 11.0 Hz, 1H), 7.57- 7.45 (m, 1H), 5.47 (d, J = 6.5 Hz, 1H), 5.26 (dd, J = 12.0, 8.0 Hz, 1H), 2.34 (q, J = 11.1 |
| | | | | Hz, 1H), 2.19 (tt, |
| | (5S,8R)-N-(5-Chloro-2-fluoro-4- | | | J = 12.2, 6.6 Hz, |
| | (trifluoromethyl)phenyl)-1,9,9- | | | 1H), 1.92 (dd, J = |
| | trifluoro-6,7,8,9-tetrahydro-5H-5,8- | | | 14.9, 8.3 Hz, 1H), |
| | epiminocyclohepta[c]pyridine-10- | | | 1.72 (ddd, J = |
| | carboxamide | | | 12.1, 9.1, 2.6 Hz, |
| | | | | 1H). |
| 295^{(d)(k)} | 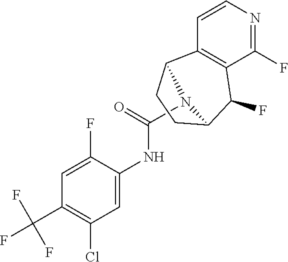 [SMILES: O=C(Nc1cc(Cl)c(C(F)(F)F)cc1F)N1[C@@H]2CC[C@H]1c1ccnc(F)c1[C@H]2F] | Method 2 435.7 at 1.68 min | 1.97 min SFC method 6 30% EtOH (0.1% Ammonia), 70% CO₂ | ¹H NMR (400 MHz, DMSO-d6) δ 9.25 (s, 1H), 8.13 (d, J = 5.2 Hz, 1H), 7.97 (d, J = 6.9 Hz, 1H), 7.72 (d, J = 11.0 Hz, 1H), 7.28 (dt, J = 5.0, 1.6 Hz, 1H), 6.21 (dd, J = 51.1, 5.8 Hz, 1H), 5.27 (d, J = 6.0 Hz, 1H), 4.94 (q, J = 4.7 Hz, 1H), 2.22-1.97 (m, 3H), 1.82-1.66 (m, 1H). |
| | (5S,8R,9R)-N-(5-Chloro-2-fluoro-4- | | | |
| | (trifluoromethyl)phenyl)-1,9- | | | |
| | difluoro-6,7,8,9-tetrahydro-5H-5,8- | | | |
| | epiminocyclohepta[c]pyridine-10- | | | |
| | carboxamide | | | |
| 296^{(d)(k)} | 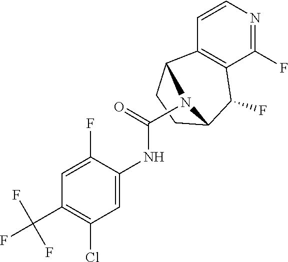 [SMILES: O=C(Nc1cc(Cl)c(C(F)(F)F)cc1F)N1[C@@H]2CC[C@H]1[C@@H](F)c1c2ccnc1F] | Method 2 435.9 at 1.68 min | 1.02 min SFC method 6 30% EtOH (0.1% Ammonia), 70% CO₂ | ¹H NMR (400 MHz, DMSO-d6) δ 9.30 (s, 1H), 8.19 (d, J = 5.0 Hz, 1H), 8.03 (d, J = 6.9 Hz, 1H), 7.79 (d, J = 11.0 Hz, 1H), 7.34 (dt, J = 5.1, 1.7 Hz, 1H), 6.27 (dd, J = 51.0, 5.8 Hz, 1H), 5.34 (d, J = 5.9 Hz, 1H), 5.00 (q, J = 4.8 Hz, 1H), 2.28-2.04 (m, 3H), 1.81 (dt, J = 10.8, 5.6 Hz, 1H). |
| | (5R,8S,9S)-N-(5-Chloro-2-fluoro-4- | | | |
| | (trifluoromethyl)phenyl)-1,9- | | | |
| | difluoro-6,7,8,9-tetrahydro-5H-5,8- | | | |
| | epiminocyclohepta[c]pyridine-10- | | | |
| | carboxamide | | | |
| 329^{(d)(h)} | 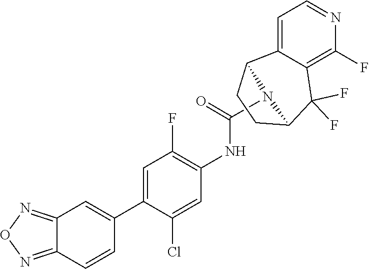 [SMILES: O=C(Nc1cc(Cl)c(-c2ccc3nonc3c2)cc1F)N1[C@H]2CC[C@@H]1C(F)(F)c1c2ccnc1F] | Method 2 503.5 at 1.73 min | 2.99 min SFC method 3 40% EtOH (0.1% Ammonia), 60% CO₂ | ¹H NMR (400 MHz, DMSO-d6) δ 9.22 (s, 1H), 8.39 (d, J = 5.1 Hz, 1H), 8.17- 8.10 (m, 2H), 7.84 (d, J = 7.2 Hz, 1H), 7.70 (dd, J = 9.3, 1.5 Hz, 1H), 7.57 (d, J = 11.1 Hz, 1H), 7.52 (d, J = 5.1 Hz, 1H), 5.48 (d, J = 6.5 Hz, 1H), 5.33- 5.17 (m, 1H), 2.41-2.28 (m, 1H), 2.27-2.12 (m, |
| | | | | 1H), 2.00-1.87 |
| | (5S,8R)-N-(4-(Benzo[c][1,2,5]- | | | (m, 1H), 1.78- |
| | oxadiazol-5-yl)-5-chloro-2- | | | 1.64 (m, 1H). |
| | fluorophenyl)-1,9,9-trifluoro-6,7,8,9- | | | |
| | tetrahydro-5H-5,8- | | | |
| | epiminocyclohepta[c]pyridine-10- | | | |
| | carboxamide | | | |
| 330^{(d)(h)} | 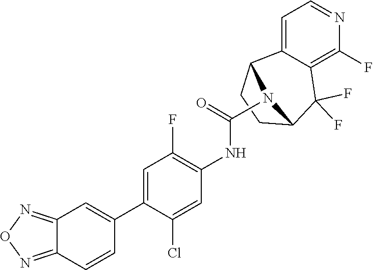 [SMILES: O=C(Nc1cc(Cl)c(-c2ccc3nonc3c2)cc1F)N1[C@@H]2CC[C@H]1C(F)(F)c1c2ccnc1F] | Method 2 503.5 at 1.73 min | 2.23 min SFC method 3 40% EtOH (0.1% Ammonia), 60% CO₂ | ¹H NMR (400 MHz, DMSO-d6) δ 9.22 (s, 1H), 8.39 (d, J = 5.1 Hz, 1H), 8.17- 8.11 (m, 2H), 7.84 (d, J = 7.2 Hz, 1H), 7.70 (dd, J = 9.3, 1.5 Hz, 1H), 7.57 (d, J = 11.1 Hz, 1H), 7.52 (d, J = 5.0 Hz, 1H), 5.48 (d, J = 6.5 Hz, 1H), 5.33- 5.20 (m, 1H), 2.43-2.28 (m, 1H), 2.28-2.12 (m, |
| | | | | 1H), 2.00-1.86 |
| | (5R,8S)-N-(4-(Benzo[c][1,2,5]- | | | (m, 1H), 1.81- |
| | oxadiazol-5-yl)-5-chloro-2- | | | 1.65 (m, 1H). |
| | fluorophenyl)-1,9,9-trifluoro-6,7,8,9- | | | |
| | tetrahydro-5H-5,8- | | | |
| | epiminocyclohepta[c]pyridine-10- | | | |
| | carboxamide | | | |

^{(a)}ES⁽⁻⁾ data reported as poor ionisation in ES⁽⁺⁾,
^{(b)}purified on Sepiatec prep SFC 50,
^{(c)}purified with a Chiralpak IC 10 x 250 mm, 5 μm particle size column,
^{(d)}purified on Waters prep 15,
^{(e)}purified with a Chiralpak IG 10 x 250 mm, 5 μm particle size column,
^{(f)}purified on Waters prep 100,
^{(g)}purified with a Phenomenex Lux ® 5 μm i-Cellulose-5, LC Column 250 x 21 mm,
^{(h)}purified with a Chiralpak IA 10 x 250 mm, 5 μm particle size column,
⁽ⁱ⁾purified with a Phenomenex Lux ® A1 5 μm, LC Column 250 x 10 mm,
^{(j)}purified with a Chiralpak IH 10 x 250 mm, 5 μm particle size column,
^{(k)}purified with a Chiralpak AY-H 10 x 250 mm, 5 μm particle size column
SFC method 1—Chiralpak IC 4.6 x 250 mm, 5 μm particle size, flow rate 4 ml/min
SFC method 2—Chiralpak IG 4.6 x 250 mm, 5 μm particle size, flow rate 4 ml/min
SFC method 3—Chiralpak IA 4.6 x 250 mm, 5 μm particle size, flow rate 4 ml/min
SFC method 4—Phenomenex Lux ® A1 4.6 x 250 mm, 5 μm particle size, flow rate 4 ml/min
SFC method 5—Chiralpak IH 4.6 x 250 mm, 5 μm particle size, flow rate 4 ml/min
SFC method 6—Chiralpak AY-H 4.6 x 250 mm, 5 μm particle size, flow rate 4 ml/min

Human GPR65 Cyclic Adenosine Monophosphate (cAMP) Homogeneous Time Resolved Fluorescence (HTRF) Antagonist Assay Procedure

IC₅₀ data was obtained by the following procedure:

1321N1 human astrocytoma cells stably expressing human recombinant GPR65 (1321N1-hrGPR65 cells, EuroscreenFast) were cultured according to the vendor's instructions.

Compounds were tested for their ability to antagonise GPR65, through measuring the concentration of cytoplasmic cAMP following treatment of the cells at a pH of 7.2 to activate GPR65 signalling and addition of the compound to be tested. The extent to which the expected rise in cAMP concentration upon GPR65 activation was suppressed by the added compound is indicative of its potency. The assay was carried out according to EuroscreenFast assay Methodology as follows.

On the day of the assay, test compounds were added to 384-well, low volume, white microtiter plates by acoustic dispensing. KRH buffer (5 mM KCl, 1.25 mM MgSO₄, 124 mM NaCl, 25 mM HEPES, 13.3 mM Glucose, 1.25 mM KH₂PO₄ and 1.45 mM CaCl₂) was adjusted to pH 6.5, pH 7.6 and pH 8.4 by adding NaOH. 1321N1-hGPR65 cells were rapidly thawed and diluted in KRH, pH 7.6 prior to centrifugation at 300×g for 5 min and resuspension in assay buffer (KRH, pH 7.6, supplemented with 1 mM 3-isobutyl-1-methylxanthine (IBMX) and 200 μM ethylenediaminetetraacetic acid (EDTA)). Cells were added to assay plates at a density of 2,000 cells per well in a volume of 5 μl. Assay plates were briefly centrifuged at 100×g and then incubated at room temperature for 30 min. Cells were stimulated by the addition of 5 μL KRH, pH 6.5, to achieve an assay pH of 7.2, while control wells received 5 μl KRH, pH 8.4 to achieve an assay pH of 7.9. Assay plates were briefly centrifuged at 100×g and then incubated at room temperature for 30 min.

Accumulation of cAMP was detected by cAMP HTRF kit (Cisbio). d2-labeled cAMP and cryptate-labeled anti-cAMP antibody in Lysis and Detection Buffer (Cisbio) were added to assay plates, and the plates were incubated at room temperature for 1 h. HTRF measurements were performed using a Pherastar FSX instrument. Acceptor and donor emission signals were measured at 665 nm and 620 nm, respectively, and HTRF ratios were calculated as signal_{665 nm}/signal_{620 nm}×10⁴. Data were normalised to high and low control values and fitted with 4-parameter logistic regression to determine hGPR65 IC50 values for the test compounds, which are shown in Table 1.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

TABLE 1
Activity of selected compounds according to the invention

| | |
|---|---|
| 1 | High |
| 2 | High |
| 3 | High |
| 4 | High |
| 5 | High |
| 6 | High |
| 7 | High |
| 8 | High |
| 9 | High |
| 10 | High |
| 11 | High |
| 12 | Medium |
| 13 | Medium |
| 14 | Medium |
| 15 | High |
| 16 | High |
| 17 | High |
| 18 | High |
| 19 | High |
| 20 | High |
| 21 | Medium |
| 22 | High |
| 23 | High |
| 24 | High |
| 25 | Medium |
| 26 | High |
| 27 | Medium |
| 28 | High |
| 29 | Low |
| 30 | Low |
| 31 | High |
| 32 | High |
| 33 | Low |
| 34 | Low |
| 35 | High |
| 36 | Low |
| 37 | High |
| 38 | Medium |
| 39 | High |
| 40 | Low |
| 41 | High |
| 42 | High |
| 43 | Medium |
| 44 | High |
| 45 | Low |
| 46 | High |
| 47 | High |
| 48 | Medium |
| 49 | High |
| 50 | Low |
| 51 | High |
| 52 | High |
| 53 | Medium |
| 54 | Low |
| 55 | High |
| 56 | Medium |
| 57 | Medium |
| 58 | Medium |
| 59 | High |
| 60 | High |
| 61 | Low |
| 62 | Low |
| 63 | High |
| 64 | High |
| 65 | High |
| 66 | High |
| 67 | High |
| 68 | Medium |
| 69 | High |
| 70 | High |
| 71 | Medium |
| 72 | Medium |
| 73 | Medium |
| 74 | High |
| 75 | High |
| 76 | High |
| 77 | High |
| 78 | Medium |
| 79 | High |
| 80 | Medium |
| 81 | High |
| 82 | High |
| 83 | Medium |
| 84 | High |
| 85 | High |
| 87 | High |
| 88 | Medium |
| 89 | Medium |
| 90 | Low |
| 91 | Medium |
| 92 | High |
| 93 | Medium |
| 95 | Low |
| 96 | High |
| 97 | High |
| 98 | High |
| 99 | Medium |
| 100 | High |
| 102 | High |
| 103 | High |
| 104 | High |
| 105 | High |
| 106 | Medium |
| 107 | Medium |
| 108 | High |
| 109 | High |
| 110 | High |
| 111 | High |
| 112 | High |
| 113 | High |
| 114 | High |
| 115 | High |
| 116 | Medium |
| 117 | Medium |
| 118 | Medium |
| 119 | Low |
| 120 | High |
| 121 | High |
| 122 | Medium |
| 123 | High |
| 124 | High |
| 125 | High |
| 126 | High |
| 127 | High |
| 128 | Medium |
| 129 | Medium |
| 130 | Medium |
| 131 | High |
| 132 | High |
| 133 | Medium |
| 134 | High |
| 135 | Medium |
| 136 | High |
| 137 | Medium |
| 138 | High |
| 139 | High |
| 140 | Medium |
| 141 | High |
| 142 | Low |
| 143 | Medium |
| 144 | High |
| 145 | High |
| 146 | Medium |
| 147 | High |
| 148 | Medium |
| 149 | Medium |
| 150 | Medium |
| 151 | Low |
| 152 | High |
| 153 | High |
| 154 | High |
| 155 | High |
| 156 | Medium |
| 157 | High |
| 158 | Low |
| 159 | High |
| 160 | Medium |
| 161 | Medium |
| 162 | High |
| 163 | High |
| 164 | High |
| 165 | High |
| 166 | High |
| 167 | High |
| 168 | High |
| 169 | High |
| 170 | High |
| 171 | High |
| 172 | High |
| 173 | High |
| 174 | Low |
| 175 | High |
| 176 | High |
| 177 | High |
| 178 | High |
| 179 | Medium |
| 180 | Medium |
| 181 | Medium |
| 182 | High |
| 183 | High |
| 184 | High |
| 185 | High |
| 186 | Medium |
| 187 | Medium |
| 188 | High |
| 189 | High |
| 190 | High |
| 191 | High |
| 192 | Medium |
| 193 | High |
| 194 | High |
| 195 | High |
| 196 | Medium |
| 197 | High |
| 198 | High |
| 199 | High |
| 200 | High |
| 201 | High |
| 202 | High |
| 203 | Medium |
| 204 | Medium |
| 205 | High |
| 206 | High |
| 207 | High |
| 208 | High |
| 209 | High |
| 210 | High |
| 211 | High |
| 212 | High |
| 213 | High |
| 214 | High |
| 215 | High |
| 216 | High |
| 217 | High |
| 218 | High |
| 219 | High |
| 220 | High |
| 221 | High |
| 222 | High |
| 223 | High |
| 224 | Medium |
| 225 | High |
| 226 | High |
| 227 | High |
| 228 | Medium |
| 229 | High |
| 230 | High |
| 231 | Medium |
| 232 | High |
| 233 | High |
| 234 | High |
| 235 | High |
| 236 | Medium |
| 237 | High |
| 238 | High |
| 239 | High |
| 240 | High |
| 241 | High |
| 242 | High |
| 243 | High |
| 244 | High |
| 245 | High |
| 246 | High |
| 247 | High |
| 248 | High |
| 249 | High |
| 250 | High |
| 251 | High |
| 252 | High |
| 253 | High |
| 254 | High |
| 255 | High |
| 256 | High |
| 257 | High |
| 258 | High |
| 259 | High |
| 260 | High |
| 261 | High |
| 262 | High |
| 263 | High |
| 264 | High |
| 265 | High |
| 266 | High |
| 267 | High |
| 268 | High |
| 269 | High |
| 270 | High |
| 271 | Medium |
| 272 | High |
| 273 | High |
| 274 | High |
| 275 | High |
| 276 | High |
| 277 | High |
| 278 | High |
| 279 | High |
| 280 | Medium |
| 281 | High |
| 282 | High |
| 283 | Medium |
| 284 | Medium |
| 285 | High |
| 286 | High |
| 287 | Low |
| 288 | Low |
| 289 | High |
| 290 | High |
| 291 | High |
| 292 | High |
| 293 | High |
| 294 | High |
| 295 | High |
| 296 | High |
| 297 | High |
| 298 | Medium |
| 299 | Medium |
| 300 | Medium |
| 301 | High |
| 302 | High |
| 303 | High |
| 304 | High |
| 305 | High |
| 306 | High |
| 307 | High |
| 308 | Medium |
| 309 | High |
| 310 | High |
| 311 | High |
| 312 | High |
| 313 | High |
| 314 | High |
| 315 | High |
| 316 | High |
| 317 | High |
| 318 | High |
| 319 | High |
| 320 | High |
| 321 | High |
| 322 | High |
| 323 | High |
| 324 | High |
| 325 | High |
| 326 | High |
| 327 | High |
| 328 | High |
| 329 | Medium |
| 330 | High |
| 331 | High |
| 332 | High |
| 333 | High |
| 334 | High |
| 335 | High |
| 336 | High |
| 337 | High |
| 338 | High |
| 339 | High |
| 340 | High |
| 341 | High |
| 342 | High |
| 343 | High |

High = IC50 <500 nM;
Medium = IC50 >500 nM and <5 μM;
Low >5 μM

### REFERENCES

Bohn, T. et al. (2018). Tumor immunoevasion via acidosis-dependent induction of regulatory tumor-associated macrophages. Nature Immunology, 1319-1326.
Damaghi, M. et al. (2013). pH Sensing and Regulation in Cancer. Frontiers in Physiology.
Gaublomme, J. et al. (2015). Single-Cell Genomics Unveils Critical Regulators of Th17 Cell Pathogenicity. Cell, 1400-1412.
Hernandez, J. (2018). GPR65, a critical regulator of Th17 cell pathogenicity, is regulated by the CRTC2/CREB pathway. The Journal of Immunology, 200 (Supplement).
Korn, T. et al. (2009). IL-17 and Th17 Cells. Annual Reviews in Immunology, 485-517.
Wang, J. et al. (2004). TDAG8 is a proton-sensing and psychosine-sensitive G-protein-coupled receptor. Journal of Biological Chemistry, 45626-45633.
Yoshida, N. et al. (2016). ICER is requisite for Th17 differentiation. Nature Communications, 12993.
Hardin, M. et al. (2014). The clinical and genetic features of COPD-asthma overlap syndrome. Eur Respir J. 2014 August; 44(2):341-50.
Kottyan, L. et al. (2009). Eosinophil viability is increased by acidic pH in a cAMP- and GPR65-dependent manner. Blood. 2009 Sep. 24; 114(13):2774-82.
Tsurumaki, H. et al (2015). Int J Mol Sci. Protective Role of Proton-Sensing TDAG8 in Lipopolysaccharide-Induced Acute Lung Injury. December 4; 16(12):28931-42.
Schultz and Wolfe (2011), Organic Letters, Intramolecular Alkene Carboamination Reactions for the Synthesis of Enantiomerically Enriched Tropane Derivatives, 13 (11), 2962-2965

1. A compound of formula (I), or a pharmaceutically acceptable salt or solvate thereof, 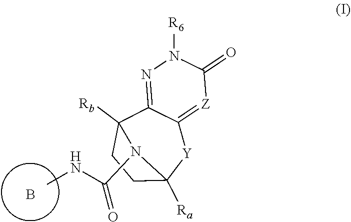
wherein:
ring B is:
a monocyclic aromatic group; or
a monocyclic or bicyclic heteroaromatic group, each of which is optionally substituted by one or more substituents selected from halo, CN, OH, alkyl, haloalkyl, cycloalkyl, halocycloalkyl, hydroxycycloalkyl, O-cycloalkyl, alkoxy, haloalkoxy, heterocycloalkyl, O-heterocycloalkyl, aryl, heteroaryl, O-aryl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, alkyl, haloalkyl, alkoxy, NHCO-alkyl, NR₁₃R_{13′}, SO₂-alkyl, CN, hydroxyalkyl, C₀NR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups;
m is an integer from 0 to 3;
p and q are each independently 0 to 3;
Z is CR₁₂;
Y is CR₁₀R_{10′}, wherein R₁₀ and R_{10′} are each independently selected from H, F, alkyl, and haloalkyl;
Rₐ and R_{b} are each independently selected from H and alkyl;
R₆ is selected from H, alkyl, cycloalkyl and hydroxyalkyl;
R₁₂ is selected from H, alkyl, haloalkyl, halo, OH and O-alkyl; and
R₁₃, R_{13′}, R₁₄, R_{14′}, R₁₅, R_{15′}, and R₁₆ are each independently selected from H, alkyl, and alkoxyalkyl.

2. A compound according to claim 1 wherein Z is CH.

3. A compound according to claim 1 wherein Rₐ and R_{b} are both H

4. A compound according to claim 1, wherein Y is CH₂.

5. A compound according to claim 1 wherein R₆ is selected from H, methyl and hydroxymethyl, and is more preferably H.

6. A compound according to claim 1 which is of formula (Ia), or a pharmaceutically acceptable salt or solvate thereof, 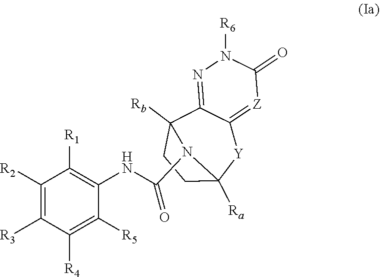
wherein:
R₁, R₄, and R₅ are each independently selected from H, ON, alkyl, alkoxy, haloalkyl, OH, and halo;
R₂ and R₃ are each independently selected from H, OH, halo, ON, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and CO₂-alkyl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, and O-aryl groups are each optionally further substituted by one or more groups independently selected from halo, haloalkyl, alkyl, alkoxy, NHCO-alkyl, NR₁₃R_{13′}, SO₂-alkyl, ON, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, and O—(CH₂)ₚ-cycloalkyl, where in the latter group, said cycloalkyl is optionally further substituted by one or more halo, haloalkyl, alkyl or alkoxy groups.

7. A compound according to claim 6, wherein R₂ and R₃ are each independently selected from H, halo, ON, alkoxy, haloalkyl, haloalkoxy, alkyl, aryl, heteroaryl, O-aryl, heterocycloalkyl, O-heterocycloalkyl, cycloalkyl, halocycloalkyl, hydroxycycloalkyl, O-cycloalkyl, NHCO-alkenyl and CO₂-alkyl, wherein said aryl, heteroaryl, heterocycloalkyl, O-cycloalkyl, and O-aryl groups are each optionally further substituted by one or more groups independently selected from halo, alkyl and alkoxy.

8. A compound according to claim 6, wherein R₂ and R₃ are each independently selected from F, Cl, Br, I, CN, C₁-C₆ haloalkyl, C₁-C₆ haloalkoxy and CO₂-alkyl, more preferably, Cl, Br, and CF₃, even more preferably Cl and CF₃.

9. A compound according to claim 6 wherein R₃ is an aryl or heteroaryl group, preferably a pyridinyl group, each of which is optionally further substituted by one or more groups independently selected from halo, haloalkyl, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, O—(CH₂)ₚ-cycloalkyl, where in the latter group, said cycloalkyl group is optionally further substituted by one or more halo, haloalkyl, alkyl or alkoxy groups.

10. A compound according to claim 6 wherein R₃ is selected from phenyl, pyridyl, pyrimidinyl, pyrazolyl, pyrazinyl, [1,2,5]thiadiazolo[3,4-b]pyridinyl, indazolyl, triazolyl, benzotriazolyl, oxoisoindolinyl, oxoindolinyl, imidazolyl, benzoxazinyl, pyrrolopyridinyl, oxotetrohydroisoquinolinyl, benzo[c][1,2,5]oxadiazolyl, benzo[c][1,2,5]thiadiazolyl, benzo[d]oxazolyl, pyridazinyl, oxazolyl, isothiazolyl, benzo[d]isooxazolyl, benzo[c]isothiazolyl, imidazo[1,5-a]pyridinyl, O-pyridinyl, CONHPh, NHCOPh, OCH₂Ph, CH₂OPh, [1,2,5]oxadiazolo[3,4-b]pyridinyl, benzo[c]isoxazolyl, or 2H-benzo[b][1,4]oxazin-3(4H)-onyl, more preferably a pyridinyl group; each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, and heteroaryl.

11. A compound according to claim 6 wherein R₃ is selected from: 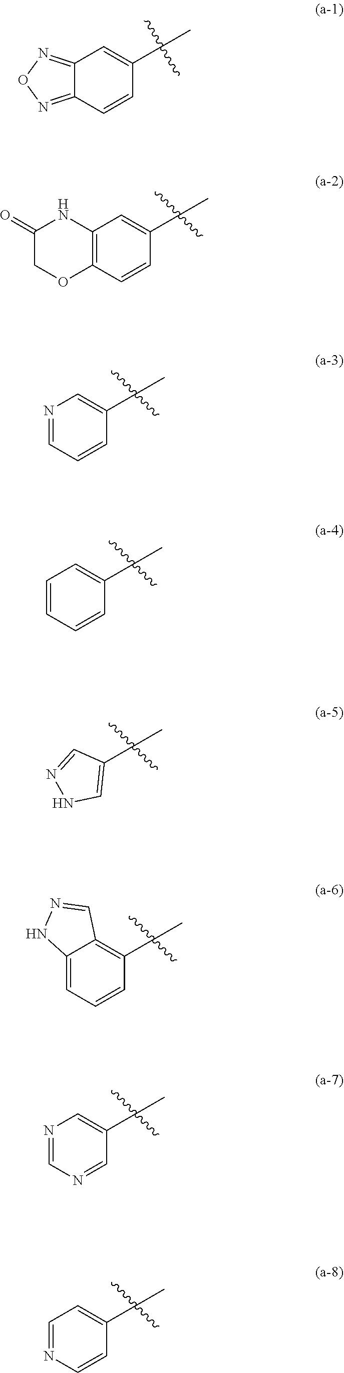 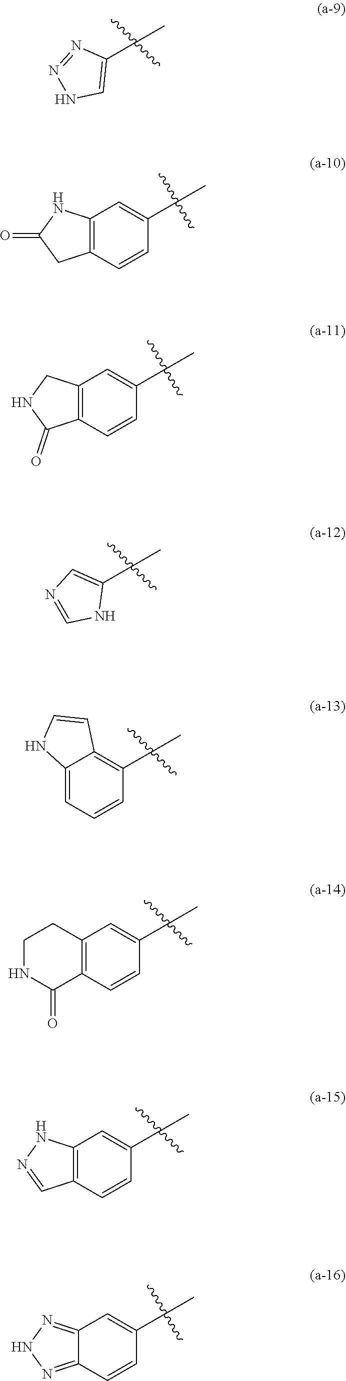 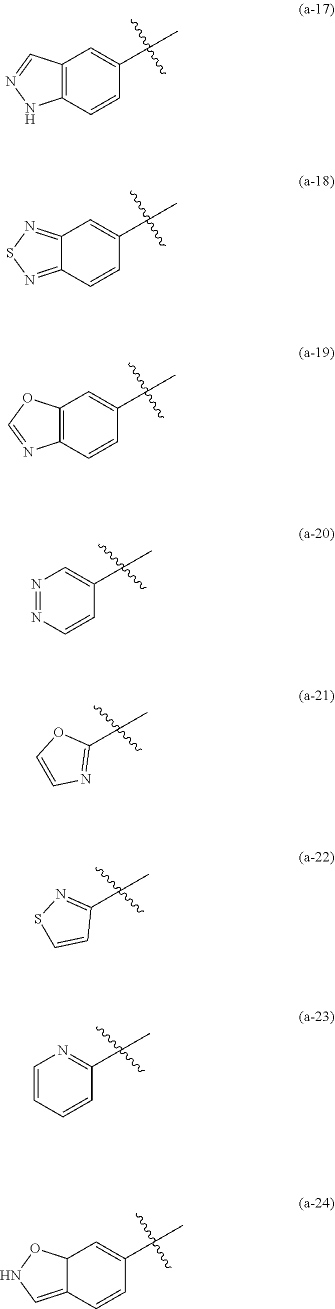 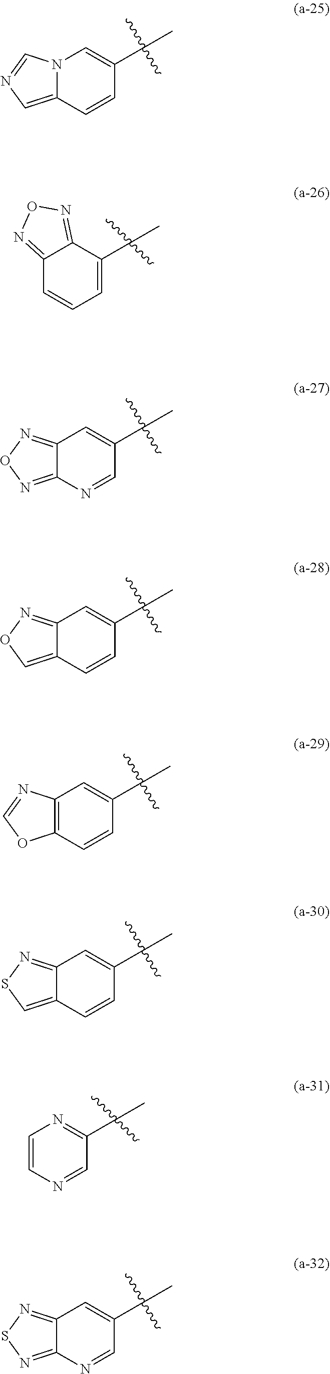 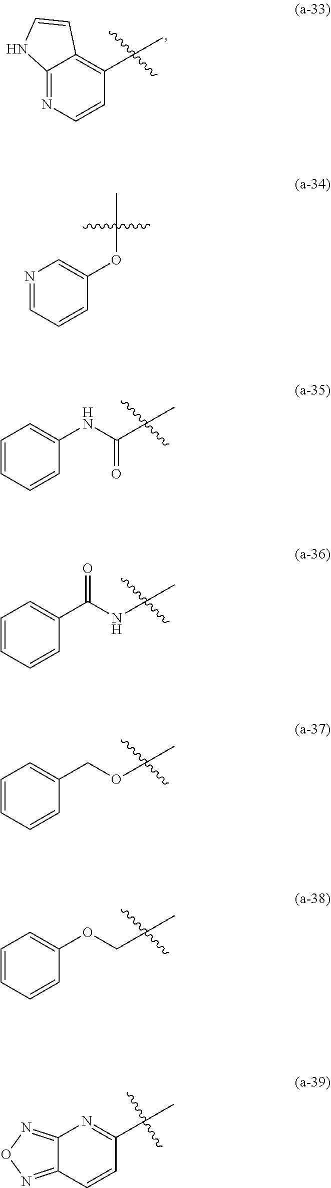
each of which is optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, haloalkoxy, O-heterocycloalkyl, heteroaryl, alkoxy-alkoxy, O—(CH₂)ₚ-cycloalkyl, and —(CH₂)_{q}—O-heteroaryl, where in the latter group, said cycloalkyl group is optionally further substituted by one or more halo, haloalkyl, alkyl or alkoxy groups.

12. A compound according to claim 6 wherein R₃ is: 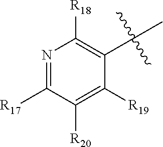
wherein:
R₁₇ is selected from H, alkyl, CN, haloalkyl, NHCO-alkyl, NR₁₃R_{13′}, alkoxy, SO₂-alkyl, halo, O—(CH₂)_{q}-heterocycloalkyl, alkoxy-alkoxy, alkoxy-alkyl, haloalkoxy, alkylamino-alkoxy, dialkylamino-alkoxy, and O—(CH₂)ₚ-cycloalkyl, wherein the cycloalkyl group is optionally substituted by one or more halo, alkyl or alkoxy groups;
R₁₈ is selected from H and halo;
R₁₉ is selected from H, alkoxy and alkoxy-alkyl; and
R₂₀ is selected from H, halo and CO₂R₁₆.

13. A compound according to claim 12 wherein:
R₁₇ is selected from H, OMe, OEt, OⁱPr, F, SO₂Me, halo, O-cyclobutyl, O-oxetanyl, and O—CH₂CF₃;
R₁₈ is selected from H and F;
R₁₉ is selected from H and MeOCH₂—;
R₂₀ is selected from H, F and CO₂Me;
R₁ is F;
R₂ is H;
R₄ is Cl or CF₃; and
R₅ is H.

14. A compound according to claim 6, wherein R₂ and R₅ are both H.

15. A compound according to claim 6, wherein R₁ is selected from H, F, Me, MeO, Cl, OH and CN, and is preferably H or F.

16. A compound according to claim 6, wherein R₄ is selected from Cl, Br, and CF₃, more preferably C₁.

17. A compound according to claim 6 wherein R₁ is F, R₂ is H, R₄ is Cl and R₅ is H.

18. A compound according to claim 1 which is of formula (Ib), or a pharmaceutically acceptable salt or solvate thereof, 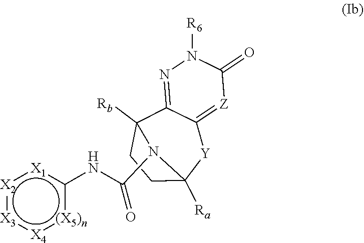
wherein where n is 0 or 1 and X₁-X₅ form a 5- or 6-membered heteroaromatic group containing at least one nitrogen atom, said heteroaromatic group being optionally substituted by one or more substituents selected from halo, CN, OH, alkyl, alkoxy, haloalkyl, haloalkoxy, aryl, heteroaryl, heterocycloalkyl, O-heterocycloalkyl, cycloalkyl, halocycloalkyl, hydroxycycloalkyl, O-cycloalkyl, NHCO-alkenyl, NHCO-aryl, —(CH₂)_{q}—O-heteroaryl, CONH-aryl, aryloxy-alkyl, O-aralkyl, and O-aryl, wherein said aryl, heteroaryl, heterocycloalkyl, O-cycloalkyl, NHCO-aryl, —(CH₂)_{q}—O-heteroaryl, CONH-aryl, aryloxy-alkyl, O-aralkyl, CO₂-alkyl, and O-aryl groups are each optionally further substituted by one or more groups independently selected from halo, haloalkyl, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, heteroaryl, alkoxy-alkoxy, and O—(CH₂)ₚ-cycloalkyl, where in the latter group, said cycloalkyl group is optionally further substituted by one or more halo, haloalkyl, alkyl or alkoxy groups.

19. A compound according to claim 18 wherein n is 1, and X₁-X₅ form a 6-membered heteroaromatic group selected from pyridin-2-yl, pyridin-3-yl, pyridin-4-yl, pyrazin-2-yl, pyrazin-3-yl, pyrimidin-2-yl, pyrimidin-4-yl, pyrimidin-5-yl, 1,2,3-triazinyl, 1,2,4-triazinyl, 1,3,5-triazinyl, 1,2,4,5-tetrazinyl and 1,2,3,4-tetrazinyl, each of which is optionally substituted by one or more substituents selected from halo, CN, alkoxy, alkyl, haloalkyl, aryl, heteroaryl, heterocycloalkyl, O-heterocycloalkyl, cycloalkyl, halocycloalkyl, hydroxycycloalkyl, O-cycloalkyl, NHCO-aryl, O-heteroaryl, CONH-aryl, aryloxy-alkyl, O-aralkyl and O-aryl, more preferably, H, halo, CN, alkoxyl, alkyl and haloalkyl, wherein said aryl, heteroaryl, heterocycloalkyl, O-cycloalkyl, NHCO-aryl, O-heteroaryl, CONH-aryl, aryloxy-alkyl, O-aralkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, O-heterocycloalkyl, and heteroaryl.

20. A compound according to claim 18 which is of formula (Ic), or a pharmaceutically acceptable salt or solvate thereof, 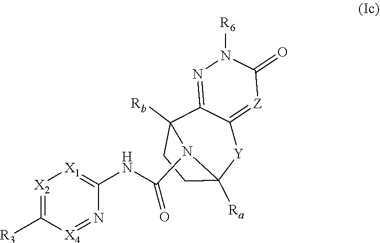
wherein:
X₁ is N or CR₁;
X₂ is N or CR₂;
X₄ is N or CR₄; and
R₁, R₂, R₃ and R₄ are each independently selected from H, halo, haloalkyl, alkyl, alkoxy, CN, aryl, heterocycloalkyl, heteroaryl and O-aryl, more preferably, H, halo, CN, alkoxyl, alkyl and haloalkyl, wherein said aryl, heteroaryl, heterocycloalkyl, and O-aryl groups are each optionally further substituted by one or more groups independently selected from halo, alkyl, alkoxy, NHCO-alkyl, NR₁₃R_{13′}, SO₂-alkyl, CN, hydroxyalkyl, CONR₁₄R_{14′}, alkyl-NR₁₅R_{15′}, heterocycloalkyl, alkyl-heterocycloalkyl, alkyl-cycloalkyl, aryl, (CH₂)ₘ—NHSO₂-alkyl, CO₂R₁₆, alkoxy-alkyl, O-cycloalkyl, haloalkoxy, and heteroaryl.

21. A compound of formula (Ib) according to claim 18, wherein X₃ is CR₃.

22. A compound according to claim 21, wherein X₁ is R₁, X₄ is R₄, wherein R₁ and R₄ are both H.

23. A compound according to claim 21, wherein X₂ is R₂, and R₂ is selected from Cl, Br, and CF₃.

24. A compound according to claim 1, which is of formula (I.1): 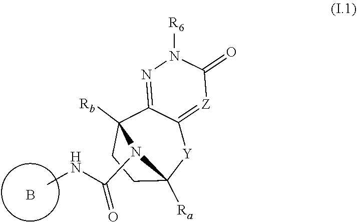
wherein B, Y, Rₐ, R_{b}, Z and R₆ are as defined in claim 1, or which is in the form of a mixture that is enantiomerically enriched with a compound of formula (I.1).

25. A compound according to claim 1, which is of formula (I.2): 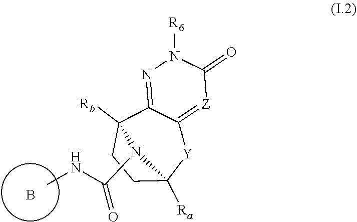
wherein B, Y, Rₐ, R_{b}, Z and R₆ are as defined in any of claim 1, or which is in the form of a mixture that is enantiomerically enriched with a compound of formula (I.2).

26. A compound according to claim 1 which is selected from the following: 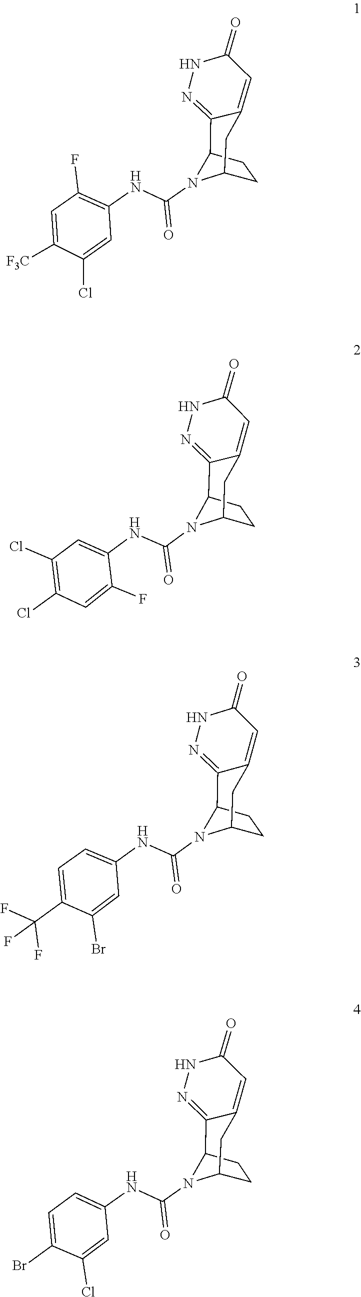 [SMILES: O=C(Nc1cc(Cl)c(C(F)(F)F)cc1F)N1C2CCC1c1n[nH]c(=O)cc1C2 ; O=C(Nc1cc(Cl)c(Cl)cc1F)N1C2CCC1c1n[nH]c(=O)cc1C2 ; O=C(Nc1ccc(C(F)(F)F)c(Br)c1)N1C2CCC1c1n[nH]c(=O)cc1C2 ; O=C(Nc1ccc(Br)c(Cl)c1)N1C2CCC1c1n[nH]c(=O)cc1C2] 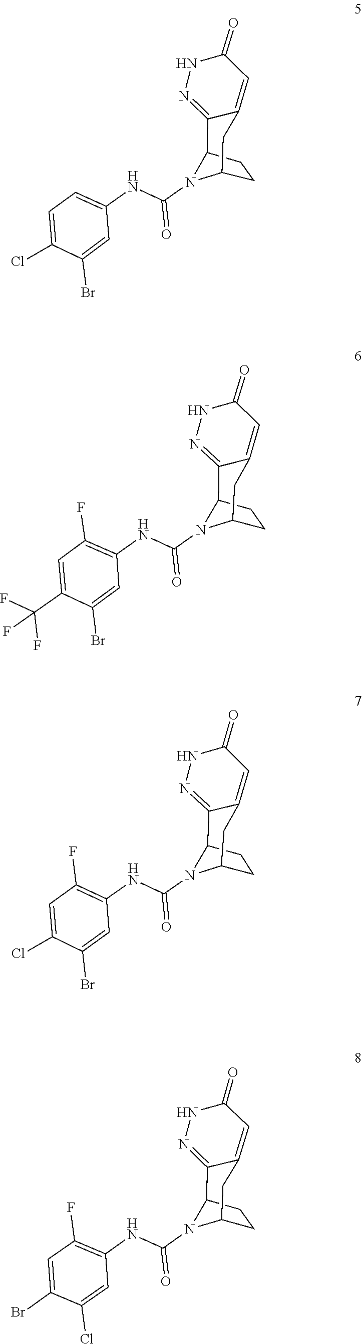 [SMILES: O=C(Nc1ccc(Cl)c(Br)c1)N1C2CCC1c1n[nH]c(=O)cc1C2 ; O=C(Nc1cc(Br)c(C(F)(F)F)cc1F)N1C2CCC1c1n[nH]c(=O)cc1C2 ; O=C(Nc1cc(Br)c(Cl)cc1F)N1C2CCC1c1n[nH]c(=O)cc1C2 ; O=C(Nc1cc(Cl)c(Br)cc1F)N1C2CCC1c1n[nH]c(=O)cc1C2] 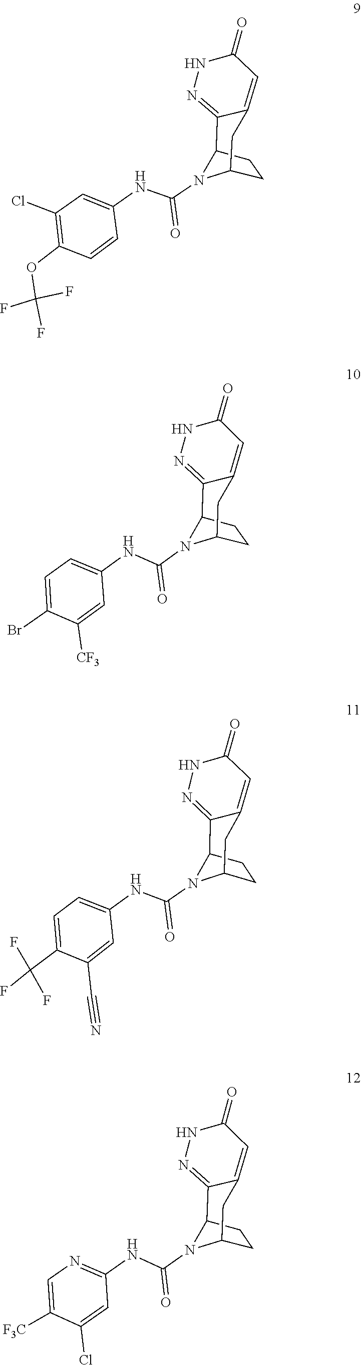 [SMILES: O=C(Nc1ccc(OC(F)(F)F)c(Cl)c1)N1C2CCC1c1n[nH]c(=O)cc1C2 ; O=C(Nc1ccc(Br)c(C(F)(F)F)c1)N1C2CCC1c1n[nH]c(=O)cc1C2 ; N#Cc1cc(NC(=O)N2C3CCC2c2n[nH]c(=O)cc2C3)ccc1C(F)(F)F ; O=C(Nc1cc(Cl)c(C(F)(F)F)cn1)N1C2CCC1c1n[nH]c(=O)cc1C2] 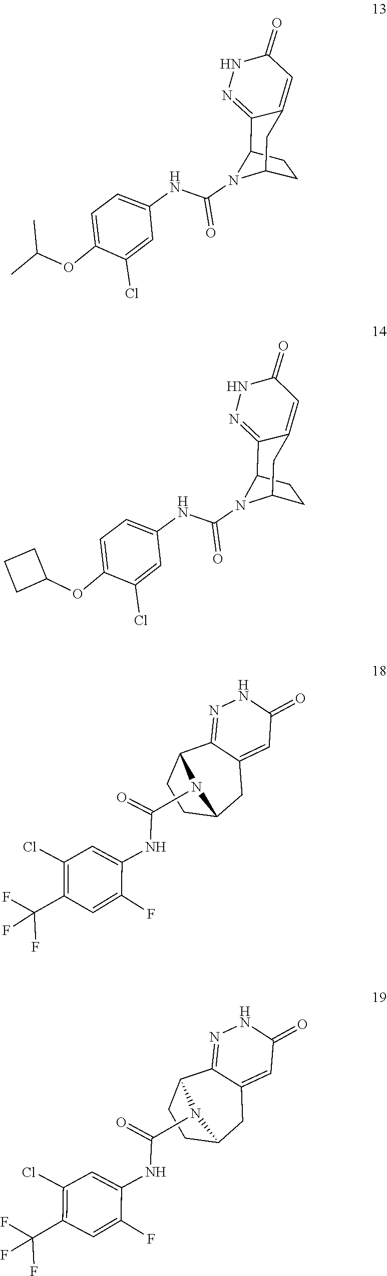 [SMILES: CC(C)Oc1ccc(NC(=O)N2C3CCC2c2n[nH]c(=O)cc2C3)cc1Cl ; O=C(Nc1ccc(OC2CCC2)c(Cl)c1)N1C2CCC1c1n[nH]c(=O)cc1C2 ; O=C(Nc1cc(Cl)c(C(F)(F)F)cc1F)N1[C@H]2CC[C@@H]1c1n[nH]c(=O)cc1C2 ; O=C(Nc1cc(Cl)c(C(F)(F)F)cc1F)N1[C@@H]2CC[C@H]1c1n[nH]c(=O)cc1C2] 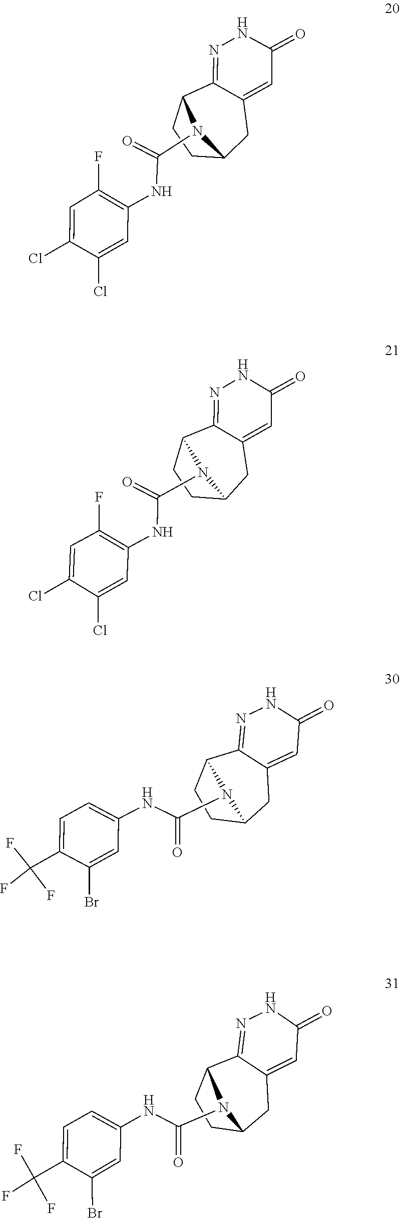 [SMILES: O=C(Nc1cc(Cl)c(Cl)cc1F)N1[C@H]2CC[C@@H]1c1n[nH]c(=O)cc1C2 ; O=C(Nc1cc(Cl)c(Cl)cc1F)N1[C@@H]2CC[C@H]1c1n[nH]c(=O)cc1C2 ; O=C(Nc1ccc(C(F)(F)F)c(Br)c1)N1[C@@H]2CC[C@H]1c1n[nH]c(=O)cc1C2 ; O=C(Nc1ccc(C(F)(F)F)c(Br)c1)N1[C@H]2CC[C@@H]1c1n[nH]c(=O)cc1C2] 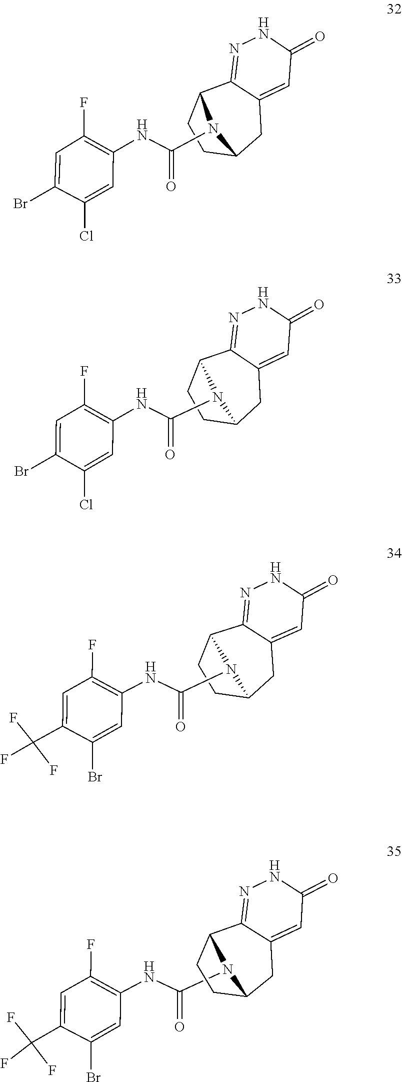 [SMILES: O=C(Nc1cc(Cl)c(Br)cc1F)N1[C@H]2CC[C@@H]1c1n[nH]c(=O)cc1C2 ; O=C(Nc1cc(Cl)c(Br)cc1F)N1[C@@H]2CC[C@H]1c1n[nH]c(=O)cc1C2 ; O=C(Nc1cc(Br)c(C(F)(F)F)cc1F)N1[C@@H]2CC[C@H]1c1n[nH]c(=O)cc1C2 ; O=C(Nc1cc(Br)c(C(F)(F)F)cc1F)N1[C@H]2CC[C@@H]1c1n[nH]c(=O)cc1C2] 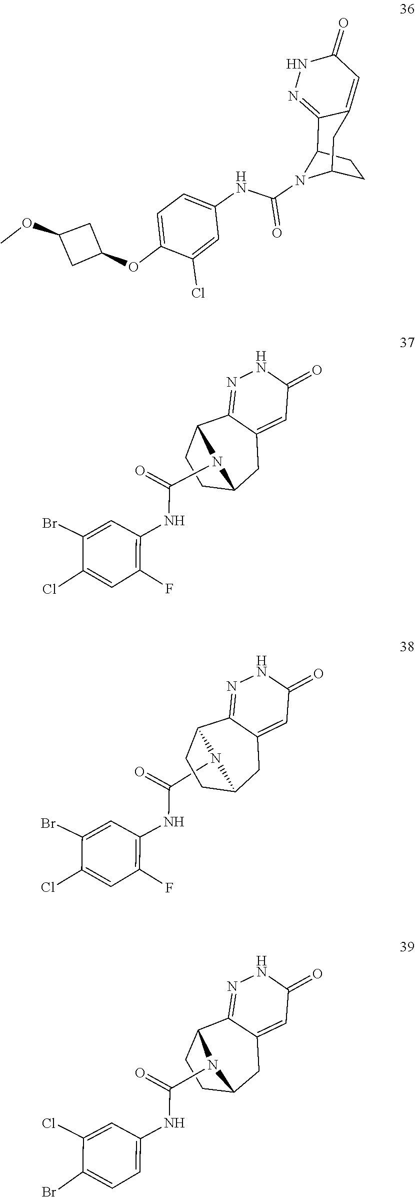 [SMILES: CO[C@H]1C[C@@H](Oc2ccc(NC(=O)N3C4CCC3c3n[nH]c(=O)cc3C4)cc2Cl)C1 ; O=C(Nc1cc(Br)c(Cl)cc1F)N1[C@H]2CC[C@@H]1c1n[nH]c(=O)cc1C2 ; O=C(Nc1cc(Br)c(Cl)cc1F)N1[C@@H]2CC[C@H]1c1n[nH]c(=O)cc1C2 ; O=C(Nc1ccc(Br)c(Cl)c1)N1[C@H]2CC[C@@H]1c1n[nH]c(=O)cc1C2] 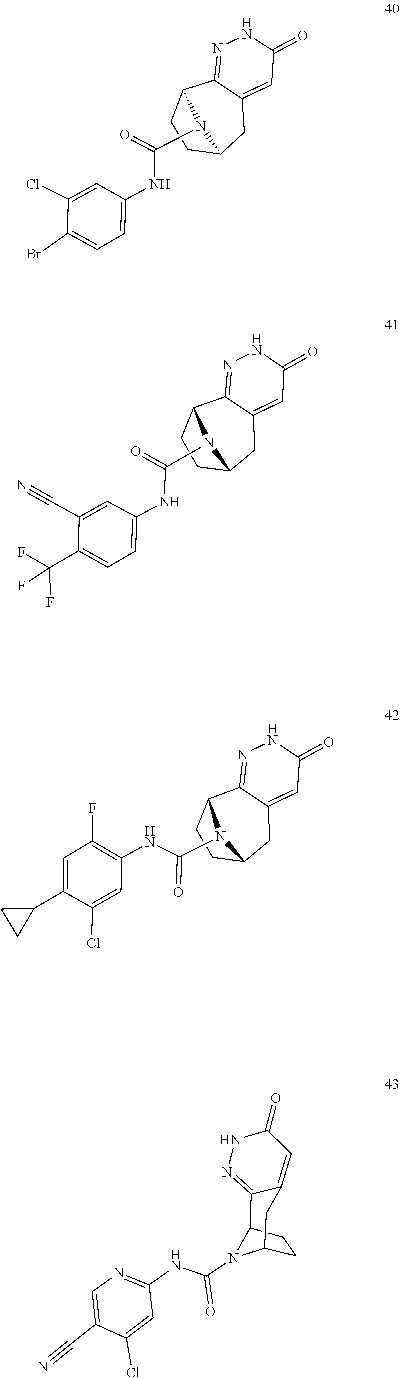 [SMILES: O=C(Nc1ccc(Br)c(Cl)c1)N1[C@@H]2CC[C@H]1c1n[nH]c(=O)cc1C2 ; N#Cc1cc(NC(=O)N2[C@H]3CC[C@@H]2c2n[nH]c(=O)cc2C3)ccc1C(F)(F)F ; O=C(Nc1cc(Cl)c(C2CC2)cc1F)N1[C@H]2CC[C@@H]1c1n[nH]c(=O)cc1C2 ; N#Cc1cnc(NC(=O)N2C3CCC2c2n[nH]c(=O)cc2C3)cc1Cl] 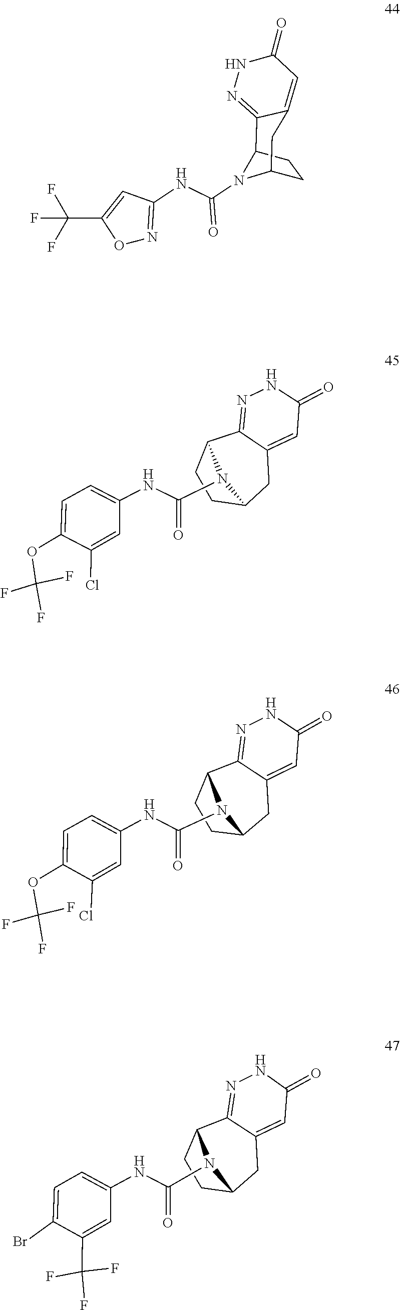 [SMILES: O=C(Nc1cc(C(F)(F)F)on1)N1C2CCC1c1n[nH]c(=O)cc1C2 ; O=C(Nc1ccc(OC(F)(F)F)c(Cl)c1)N1[C@@H]2CC[C@H]1c1n[nH]c(=O)cc1C2 ; O=C(Nc1ccc(OC(F)(F)F)c(Cl)c1)N1[C@H]2CC[C@@H]1c1n[nH]c(=O)cc1C2 ; O=C(Nc1ccc(Br)c(C(F)(F)F)c1)N1[C@H]2CC[C@@H]1c1n[nH]c(=O)cc1C2] 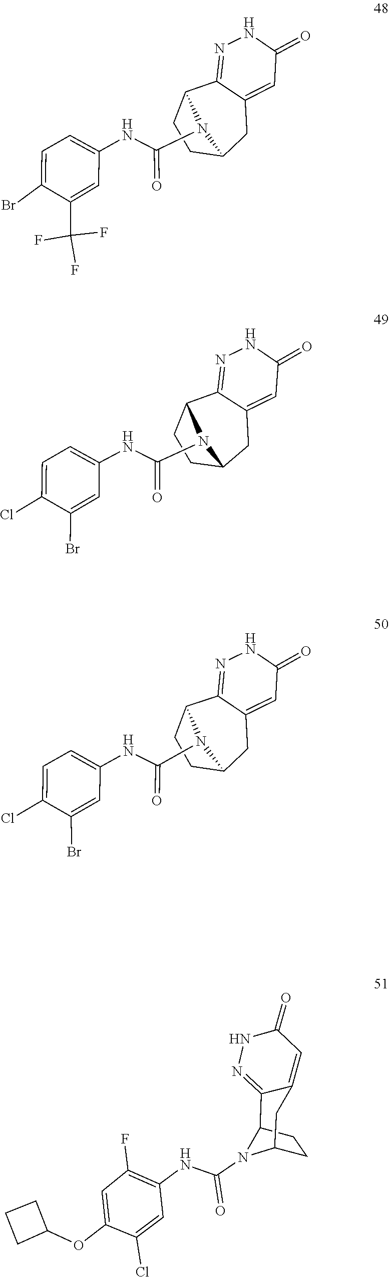 [SMILES: O=C(Nc1ccc(Br)c(C(F)(F)F)c1)N1[C@@H]2CC[C@H]1c1n[nH]c(=O)cc1C2 ; O=C(Nc1ccc(Cl)c(Br)c1)N1[C@H]2CC[C@@H]1c1n[nH]c(=O)cc1C2 ; O=C(Nc1ccc(Cl)c(Br)c1)N1[C@@H]2CC[C@H]1c1n[nH]c(=O)cc1C2 ; O=C(Nc1cc(Cl)c(OC2CCC2)cc1F)N1C2CCC1c1n[nH]c(=O)cc1C2] 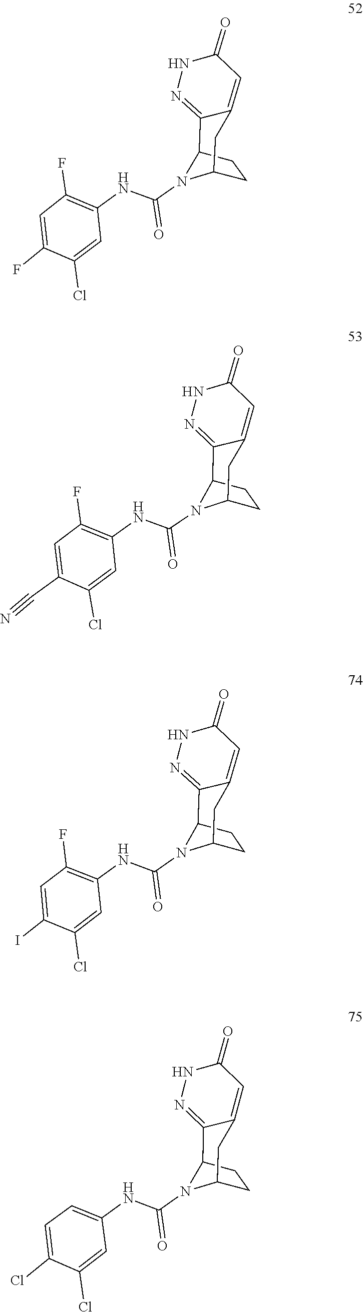 [SMILES: O=C(Nc1cc(Cl)c(F)cc1F)N1C2CCC1c1n[nH]c(=O)cc1C2 ; N#Cc1cc(F)c(NC(=O)N2C3CCC2c2n[nH]c(=O)cc2C3)cc1Cl ; O=C(Nc1cc(Cl)c(I)cc1F)N1C2CCC1c1n[nH]c(=O)cc1C2 ; O=C(Nc1ccc(Cl)c(Cl)c1)N1C2CCC1c1n[nH]c(=O)cc1C2] 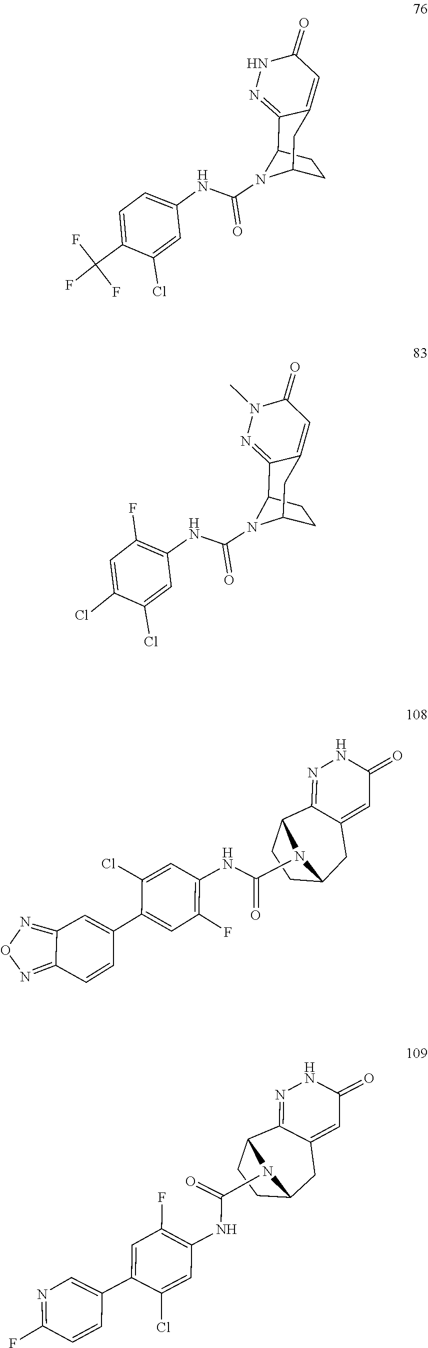 [SMILES: O=C(Nc1ccc(C(F)(F)F)c(Cl)c1)N1C2CCC1c1n[nH]c(=O)cc1C2 ; Cn1nc2c(cc1=O)CC1CCC2N1C(=O)Nc1cc(Cl)c(Cl)cc1F ; O=C(Nc1cc(Cl)c(-c2ccc3nonc3c2)cc1F)N1[C@H]2CC[C@@H]1c1n[nH]c(=O)cc1C2 ; O=C(Nc1cc(Cl)c(-c2ccc(F)nc2)cc1F)N1[C@H]2CC[C@@H]1c1n[nH]c(=O)cc1C2] 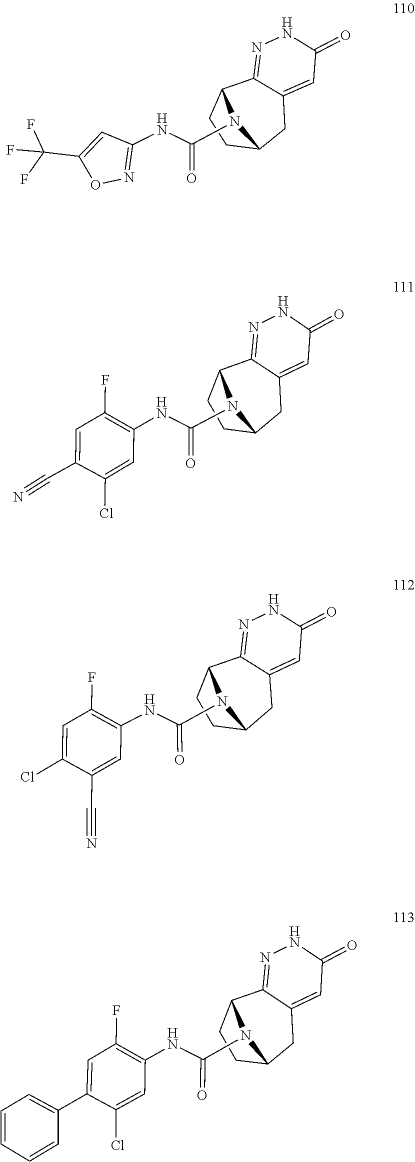 [SMILES: O=C(Nc1cc(C(F)(F)F)on1)N1[C@H]2CC[C@@H]1c1n[nH]c(=O)cc1C2 ; N#Cc1cc(F)c(NC(=O)N2[C@H]3CC[C@@H]2c2n[nH]c(=O)cc2C3)cc1Cl ; N#Cc1cc(NC(=O)N2[C@H]3CC[C@@H]2c2n[nH]c(=O)cc2C3)c(F)cc1Cl ; O=C(Nc1cc(Cl)c(-c2ccccc2)cc1F)N1[C@H]2CC[C@@H]1c1n[nH]c(=O)cc1C2] 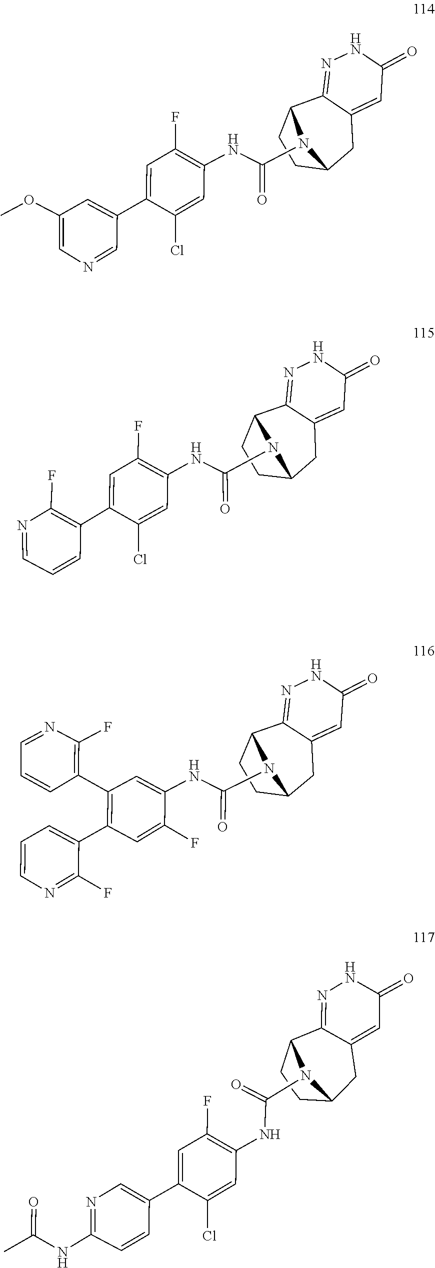 [SMILES: COc1cncc(-c2cc(F)c(NC(=O)N3[C@H]4CC[C@@H]3c3n[nH]c(=O)cc3C4)cc2Cl)c1 ; O=C(Nc1cc(Cl)c(-c2cccnc2F)cc1F)N1[C@H]2CC[C@@H]1c1n[nH]c(=O)cc1C2 ; O=C(Nc1cc(-c2cccnc2F)c(-c2cccnc2F)cc1F)N1[C@H]2CC[C@@H]1c1n[nH]c(=O)cc1C2 ; CC[C@H]1Cc2cc(=O)[nH]nc2[C@@H](CC)N1C(=O)Nc1cc(Cl)c(-c2ccc(NC(C)=O)nc2)cc1F] 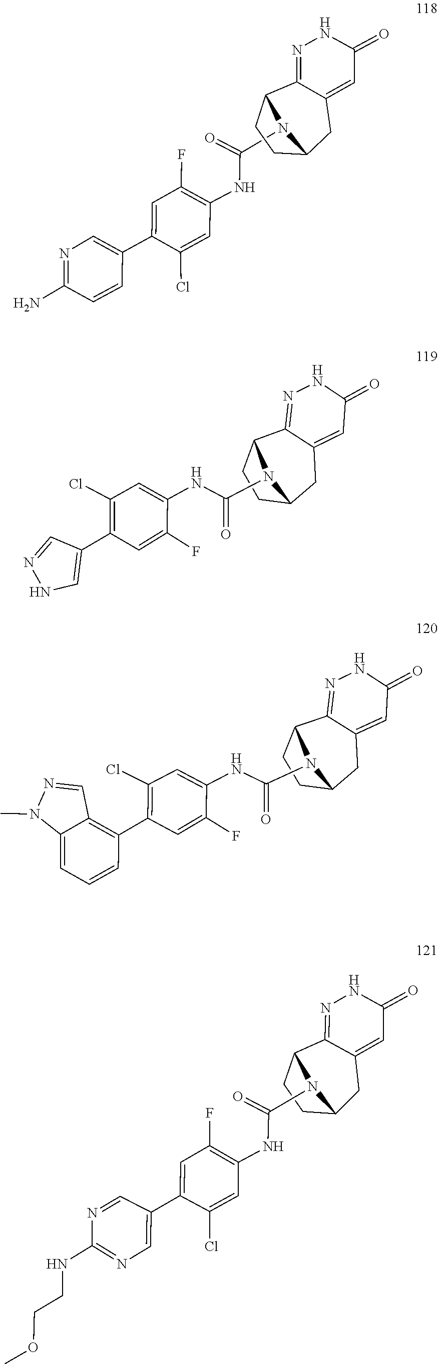 [SMILES: Nc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2cn[nH]c2)cc1F)N1[C@H]2CC[C@@H]1c1n[nH]c(=O)cc1C2 ; Cn1ncc2c(-c3cc(F)c(NC(=O)N4[C@H]5CC[C@@H]4c4n[nH]c(=O)cc4C5)cc3Cl)cccc21 ; COCCNc1ncc(-c2cc(F)c(NC(=O)N3[C@H]4CC[C@@H]3c3n[nH]c(=O)cc3C4)cc2Cl)cn1] 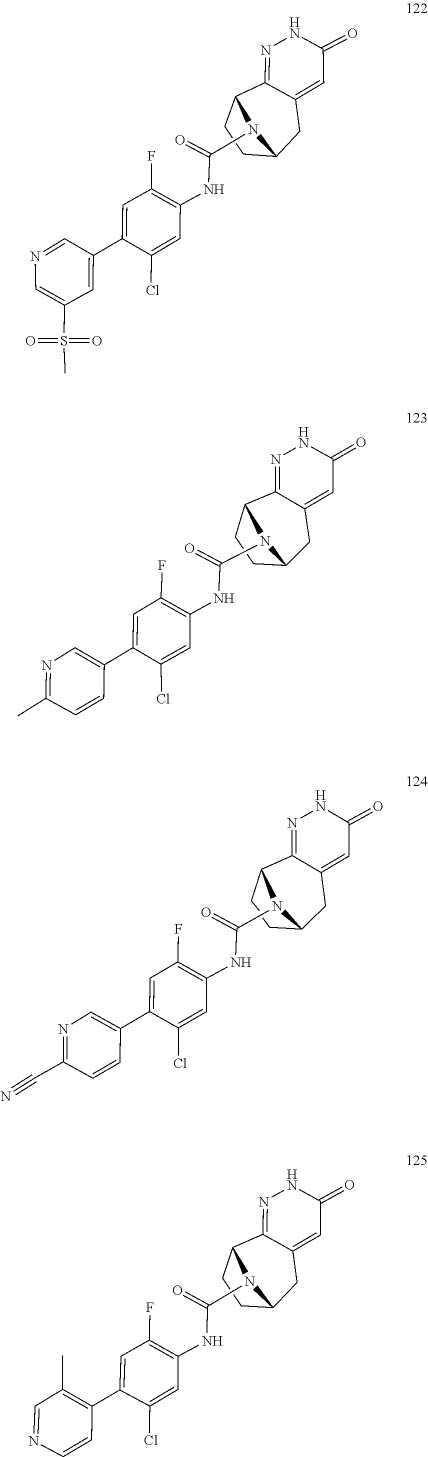 [SMILES: CS(=O)(=O)c1cncc(-c2cc(F)c(NC(=O)N3[C@H]4CC[C@@H]3c3n[nH]c(=O)cc3C4)cc2Cl)c1 ; Cc1ccc(-c2cc(F)c(NC(=O)N3[C@H]4CC[C@@H]3c3n[nH]c(=O)cc3C4)cc2Cl)cn1 ; CC[C@H]1Cc2cc(=O)[nH]nc2[C@@H](CC)N1C(=O)Nc1cc(Cl)c(-c2ccc(C#N)nc2)cc1F ; Cc1cnccc1-c1cc(F)c(NC(=O)N2[C@H]3CC[C@@H]2c2n[nH]c(=O)cc2C3)cc1Cl] 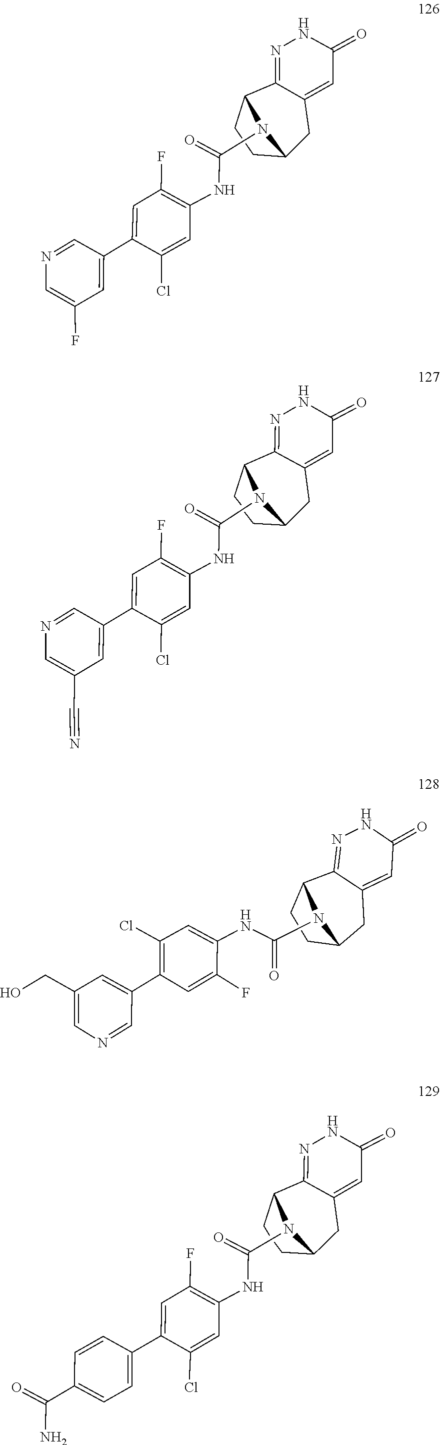 [SMILES: O=C(Nc1cc(Cl)c(-c2cncc(F)c2)cc1F)N1[C@H]2CC[C@@H]1c1n[nH]c(=O)cc1C2 ; CC[C@H]1Cc2cc(=O)[nH]nc2[C@@H](CC)N1C(=O)Nc1cc(Cl)c(-c2cncc(C#N)c2)cc1F ; O=C(Nc1cc(Cl)c(-c2cncc(CO)c2)cc1F)N1[C@H]2CC[C@@H]1c1n[nH]c(=O)cc1C2 ; CC[C@H]1Cc2cc(=O)[nH]nc2[C@@H](CC)N1C(=O)Nc1cc(Cl)c(-c2ccc(C(N)=O)cc2)cc1F] 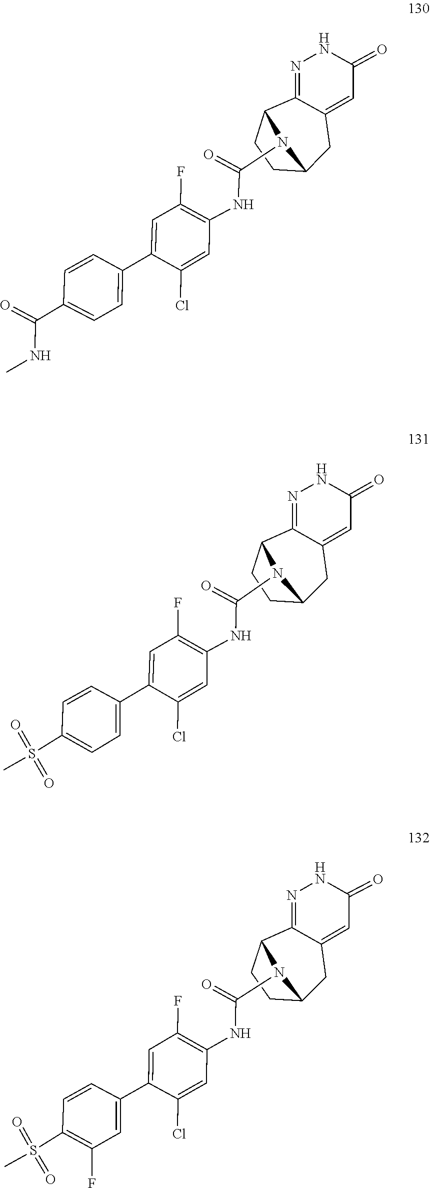 [SMILES: CNC(=O)c1ccc(-c2cc(F)c(NC(=O)N3[C@H]4CC[C@@H]3c3n[nH]c(=O)cc3C4)cc2Cl)cc1 ; CS(=O)(=O)c1ccc(-c2cc(F)c(NC(=O)N3[C@H]4CC[C@@H]3c3n[nH]c(=O)cc3C4)cc2Cl)cc1 ; CS(=O)(=O)c1ccc(-c2cc(F)c(NC(=O)N3[C@H]4CC[C@@H]3c3n[nH]c(=O)cc3C4)cc2Cl)cc1F] 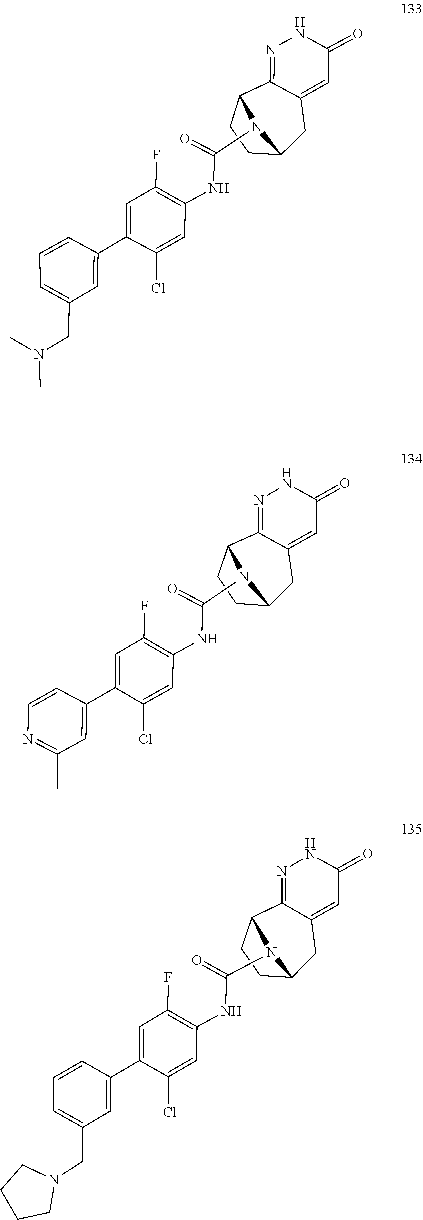 [SMILES: CN(C)Cc1cccc(-c2cc(F)c(NC(=O)N3[C@H]4CC[C@@H]3c3n[nH]c(=O)cc3C4)cc2Cl)c1 ; Cc1cc(-c2cc(F)c(NC(=O)N3[C@H]4CC[C@@H]3c3n[nH]c(=O)cc3C4)cc2Cl)ccn1 ; O=C(Nc1cc(Cl)c(-c2cccc(CN3CCCC3)c2)cc1F)N1[C@H]2CC[C@@H]1c1n[nH]c(=O)cc1C2] 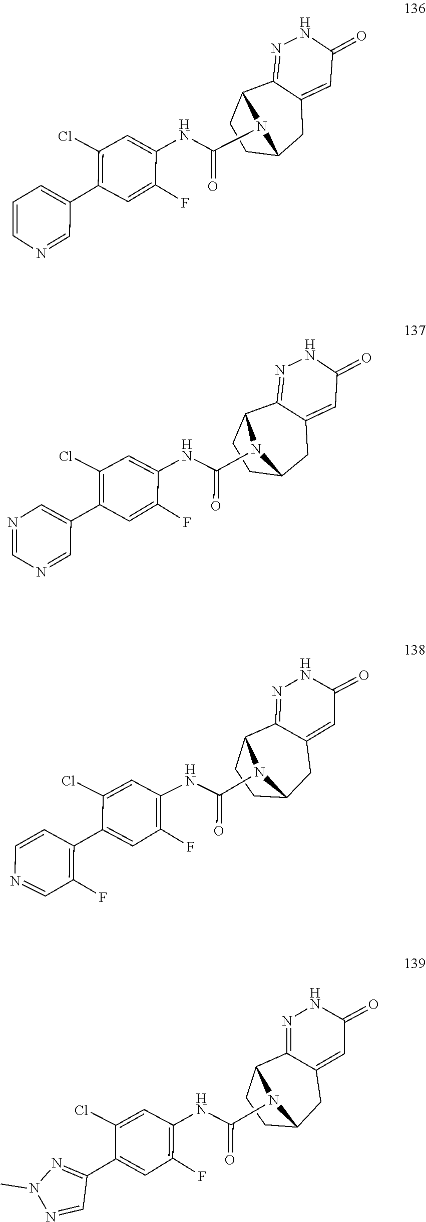 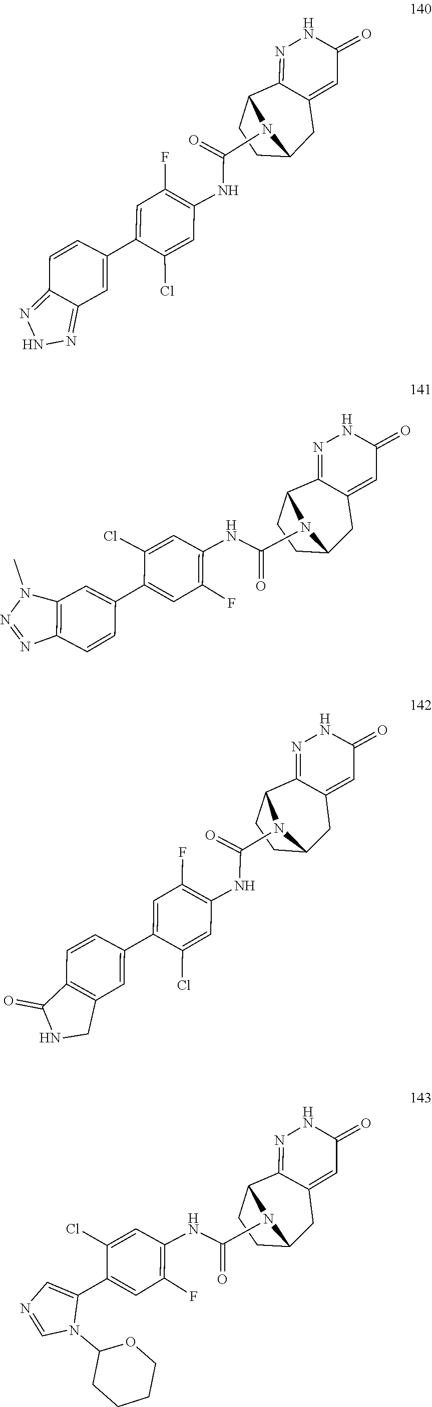 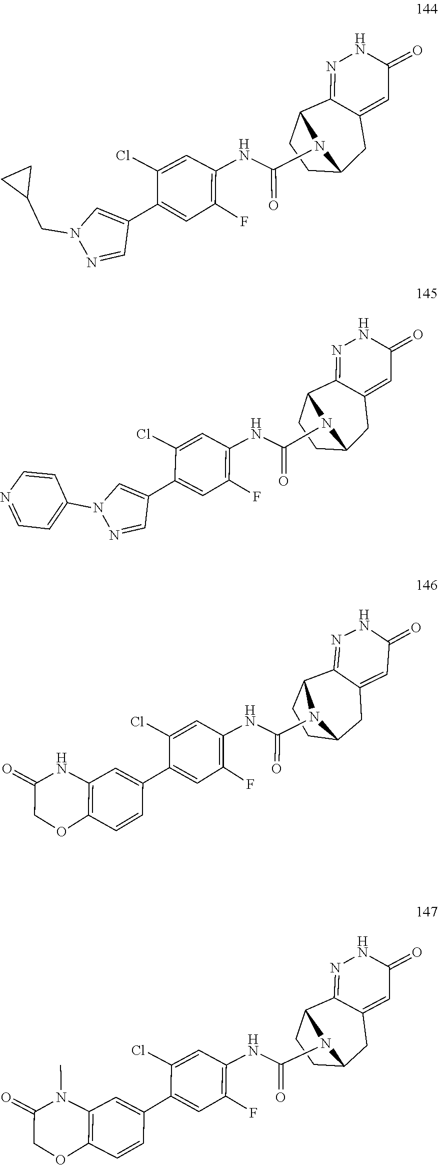 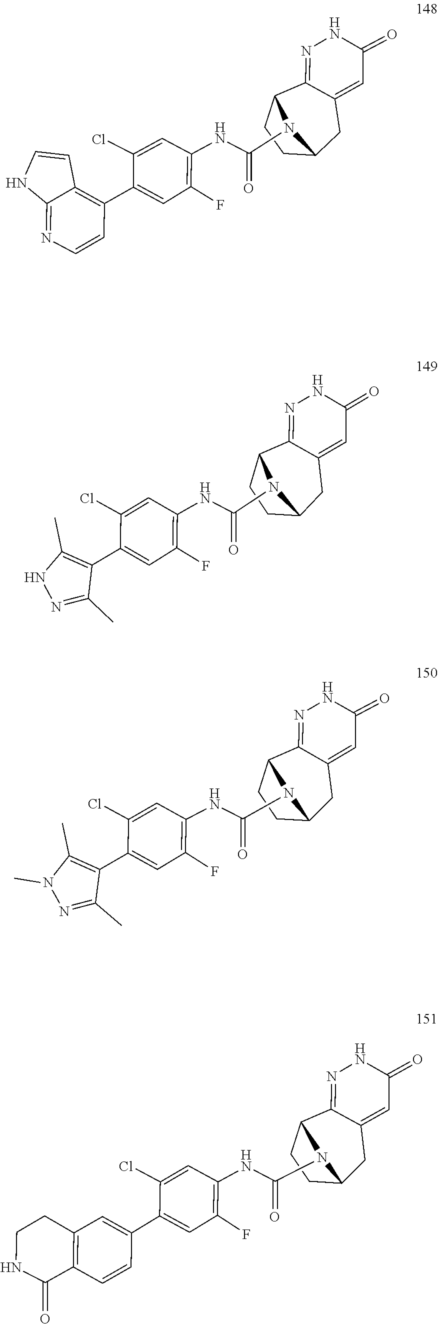 [SMILES: O=C(Nc1cc(Cl)c(-c2ccnc3[nH]ccc23)cc1F)N1[C@H]2CC[C@@H]1c1n[nH]c(=O)cc1C2 ; Cc1n[nH]c(C)c1-c1cc(F)c(NC(=O)N2[C@H]3CC[C@@H]2c2n[nH]c(=O)cc2C3)cc1Cl ; Cc1nn(C)c(C)c1-c1cc(F)c(NC(=O)N2[C@H]3CC[C@@H]2c2n[nH]c(=O)cc2C3)cc1Cl ; O=C1NCCc2cc(-c3cc(F)c(NC(=O)N4[C@H]5CC[C@@H]4c4n[nH]c(=O)cc4C5)cc3Cl)ccc21] 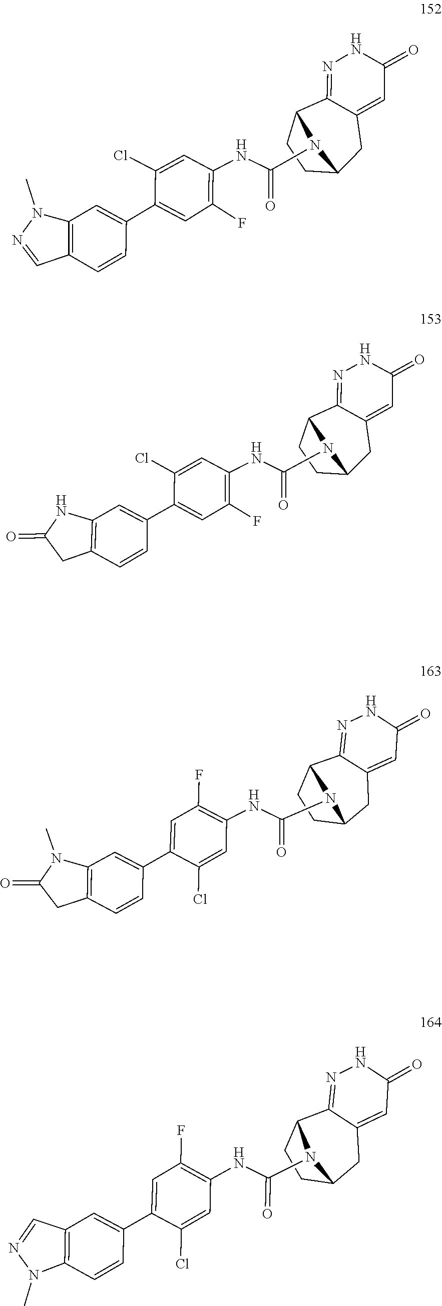 [SMILES: Cn1ncc2ccc(-c3cc(F)c(NC(=O)N4[C@H]5CC[C@@H]4c4n[nH]c(=O)cc4C5)cc3Cl)cc21 ; O=C1Cc2ccc(-c3cc(F)c(NC(=O)N4[C@H]5CC[C@@H]4c4n[nH]c(=O)cc4C5)cc3Cl)cc2N1 ; CN1C(=O)Cc2ccc(-c3cc(F)c(NC(=O)N4[C@H]5CC[C@@H]4c4n[nH]c(=O)cc4C5)cc3Cl)cc21 ; Cn1ncc2cc(-c3cc(F)c(NC(=O)N4[C@H]5CC[C@@H]4c4n[nH]c(=O)cc4C5)cc3Cl)ccc21] 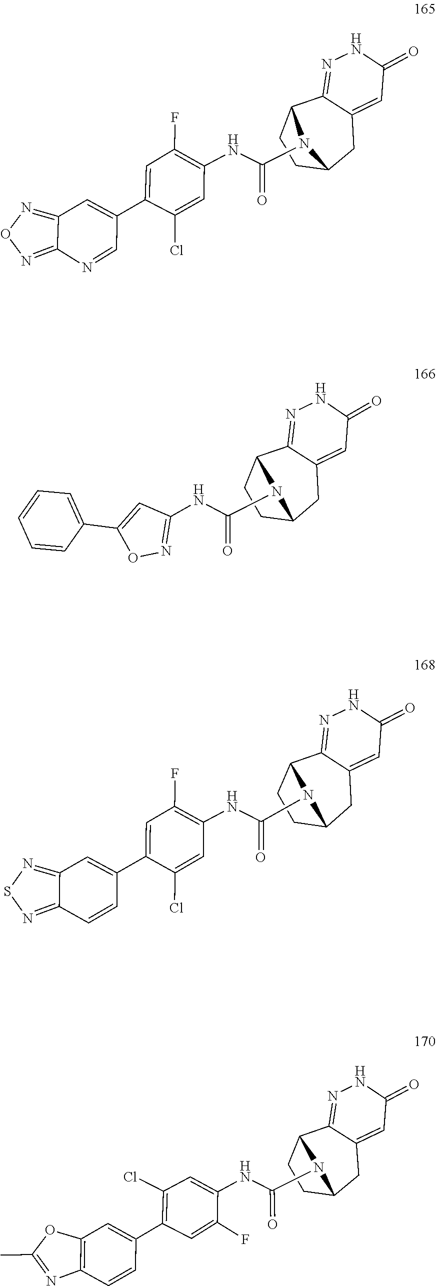 [SMILES: CC[C@H]1Cc2cc(=O)[nH]nc2[C@@H](CC)N1C(=O)Nc1cc(Cl)c(-c2cnc3nonc3c2)cc1F ; O=C(Nc1cc(-c2ccccc2)on1)N1[C@H]2CC[C@@H]1c1n[nH]c(=O)cc1C2 ; O=C(Nc1cc(Cl)c(-c2ccc3nsnc3c2)cc1F)N1[C@H]2CC[C@@H]1c1n[nH]c(=O)cc1C2 ; Cc1nc2ccc(-c3cc(F)c(NC(=O)N4[C@H]5CC[C@@H]4c4n[nH]c(=O)cc4C5)cc3Cl)cc2o1] 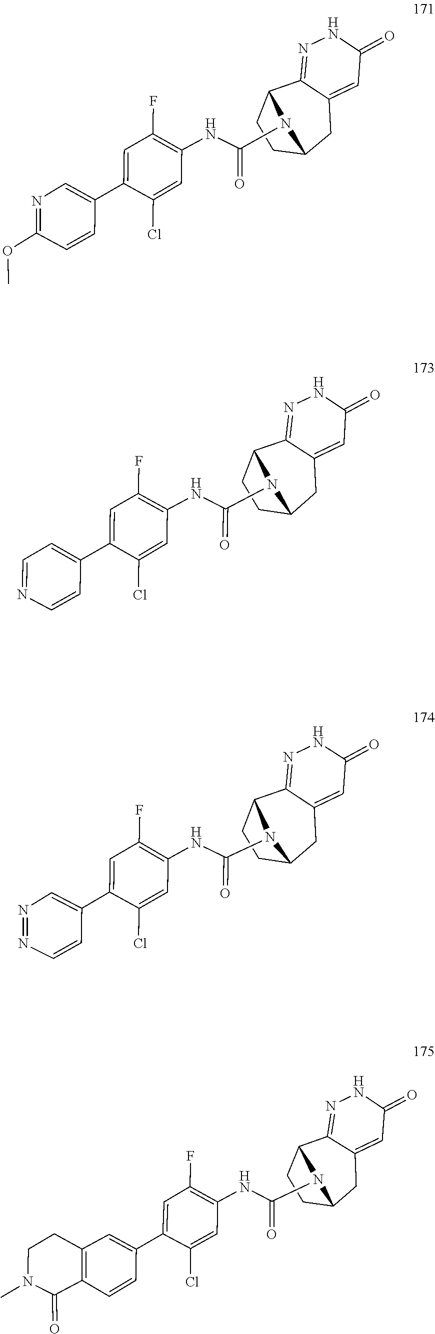 [SMILES: COc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccncc2)cc1F)N1[C@H]2CC[C@@H]1c1n[nH]c(=O)cc1C2 ; O=C(Nc1cc(Cl)c(-c2ccnnc2)cc1F)N1[C@H]2CC[C@@H]1c1n[nH]c(=O)cc1C2 ; CN1CCc2cc(-c3cc(F)c(NC(=O)N4[C@H]5CC[C@@H]4c4n[nH]c(=O)cc4C5)cc3Cl)ccc2C1=O] 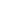 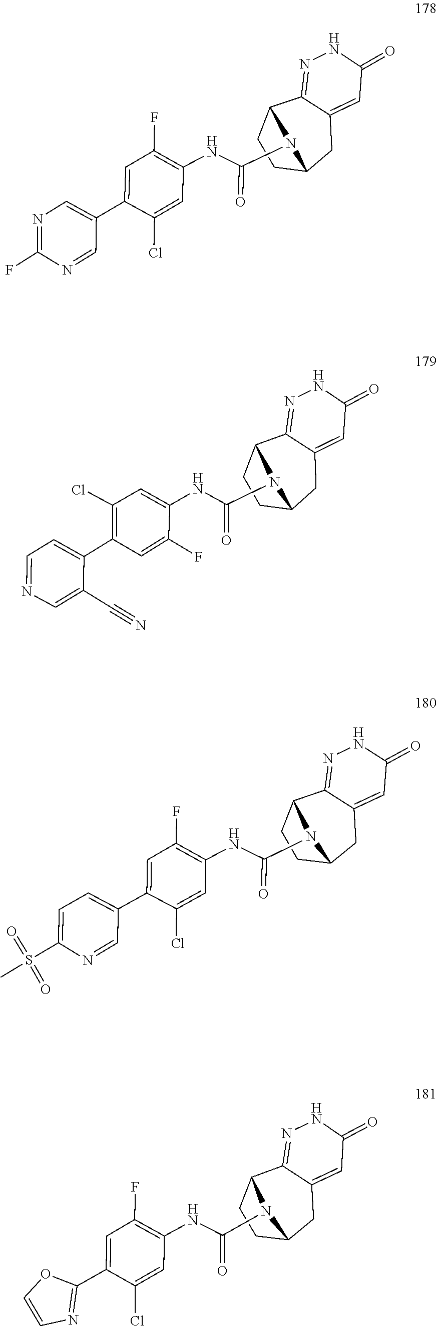 [SMILES: O=C(Nc1cc(Cl)c(-c2cnc(F)nc2)cc1F)N1[C@H]2CC[C@@H]1c1n[nH]c(=O)cc1C2 ; N#Cc1cnccc1-c1cc(F)c(NC(=O)N2[C@H]3CC[C@@H]2c2n[nH]c(=O)cc2C3)cc1Cl ; CS(=O)(=O)c1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ncco2)cc1F)N1[C@H]2CC[C@@H]1c1n[nH]c(=O)cc1C2] 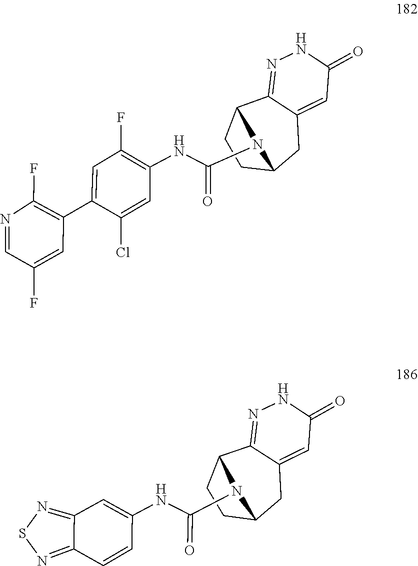 [SMILES: O=C(Nc1cc(Cl)c(-c2cc(F)cnc2F)cc1F)N1[C@H]2CC[C@@H]1c1n[nH]c(=O)cc1C2 ; O=C(Nc1ccc2nsnc2c1)N1[C@H]2CC[C@@H]1c1n[nH]c(=O)cc1C2] 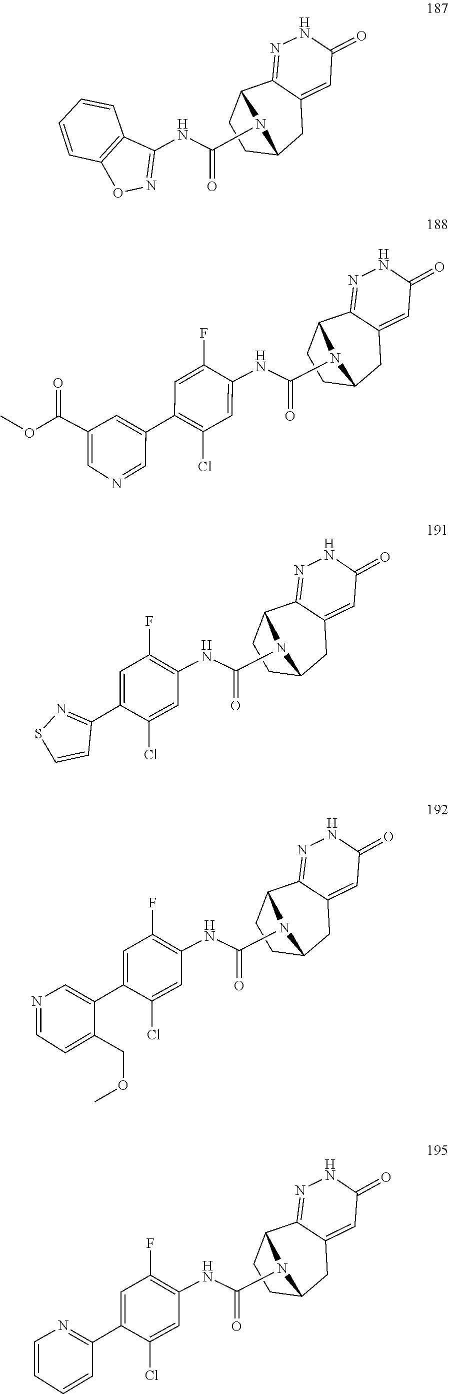 [SMILES: O=C(Nc1noc2ccccc12)N1[C@H]2CC[C@@H]1c1n[nH]c(=O)cc1C2 ; COC(=O)c1cncc(-c2cc(F)c(NC(=O)N3[C@H]4CC[C@@H]3c3n[nH]c(=O)cc3C4)cc2Cl)c1 ; O=C(Nc1cc(Cl)c(-c2ccsn2)cc1F)N1[C@H]2CC[C@@H]1c1n[nH]c(=O)cc1C2 ; COCc1ccncc1-c1cc(F)c(NC(=O)N2[C@H]3CC[C@@H]2c2n[nH]c(=O)cc2C3)cc1Cl ; O=C(Nc1cc(Cl)c(-c2ccccn2)cc1F)N1[C@H]2CC[C@@H]1c1n[nH]c(=O)cc1C2] 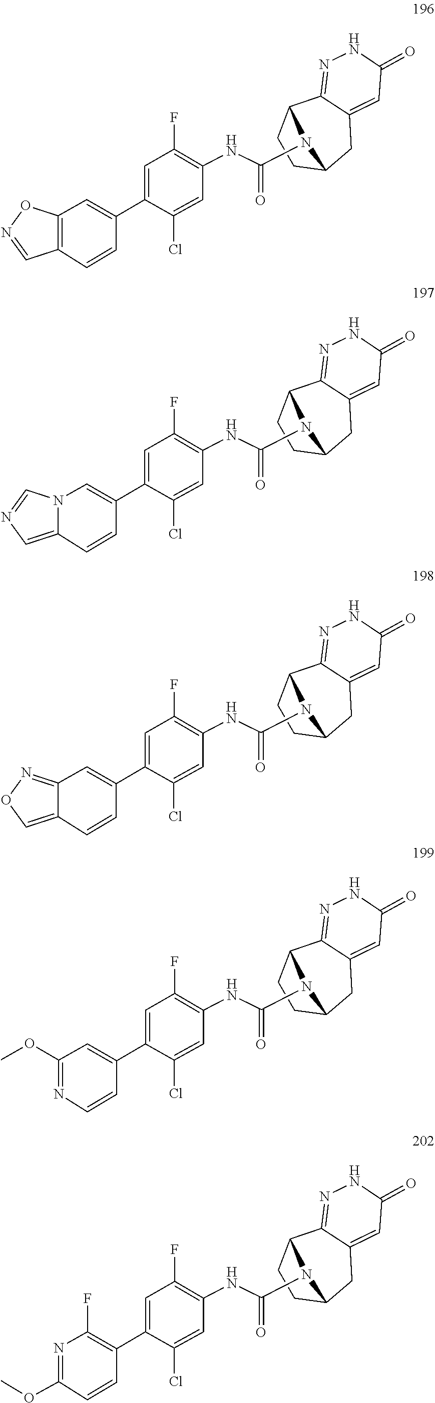 [SMILES: O=C(Nc1cc(Cl)c(-c2ccc3cnoc3c2)cc1F)N1[C@H]2CC[C@@H]1c1n[nH]c(=O)cc1C2 ; O=C(Nc1cc(Cl)c(-c2ccc3cncn3c2)cc1F)N1[C@H]2CC[C@@H]1c1n[nH]c(=O)cc1C2 ; O=C(Nc1cc(Cl)c(-c2ccc3conc3c2)cc1F)N1[C@H]2CC[C@@H]1c1n[nH]c(=O)cc1C2 ; COc1cc(-c2cc(F)c(NC(=O)N3[C@H]4CC[C@@H]3c3n[nH]c(=O)cc3C4)cc2Cl)ccn1 ; COc1ccc(-c2cc(F)c(NC(=O)N3[C@H]4CC[C@@H]3c3n[nH]c(=O)cc3C4)cc2Cl)c(F)n1] 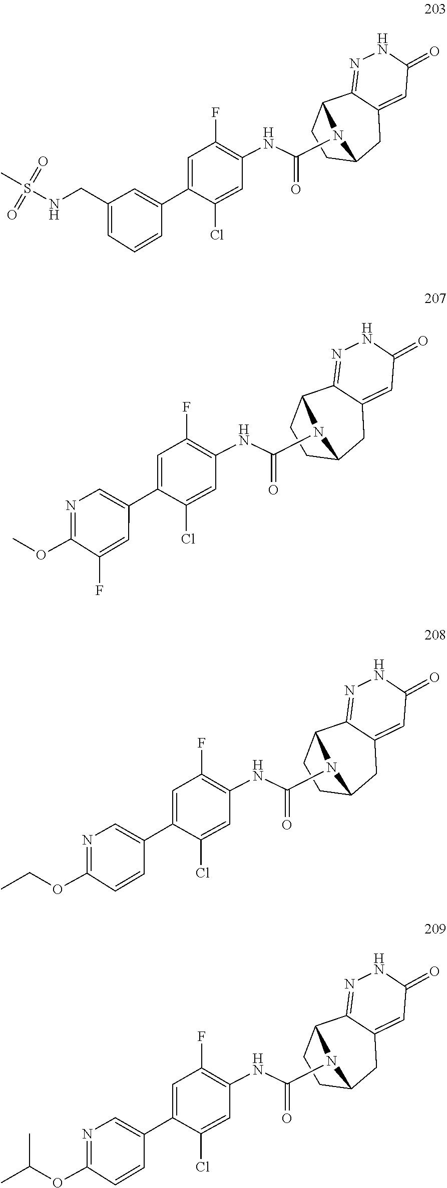 [SMILES: CS(=O)(=O)NCc1cccc(-c2cc(F)c(NC(=O)N3[C@H]4CC[C@@H]3c3n[nH]c(=O)cc3C4)cc2Cl)c1 ; COc1ncc(-c2cc(F)c(NC(=O)N3[C@H]4CC[C@@H]3c3n[nH]c(=O)cc3C4)cc2Cl)cc1F ; CCOc1ccc(-c2cc(F)c(NC(=O)N3[C@H]4CC[C@@H]3c3n[nH]c(=O)cc3C4)cc2Cl)cn1 ; CC(C)Oc1ccc(-c2cc(F)c(NC(=O)N3[C@H]4CC[C@@H]3c3n[nH]c(=O)cc3C4)cc2Cl)cn1] 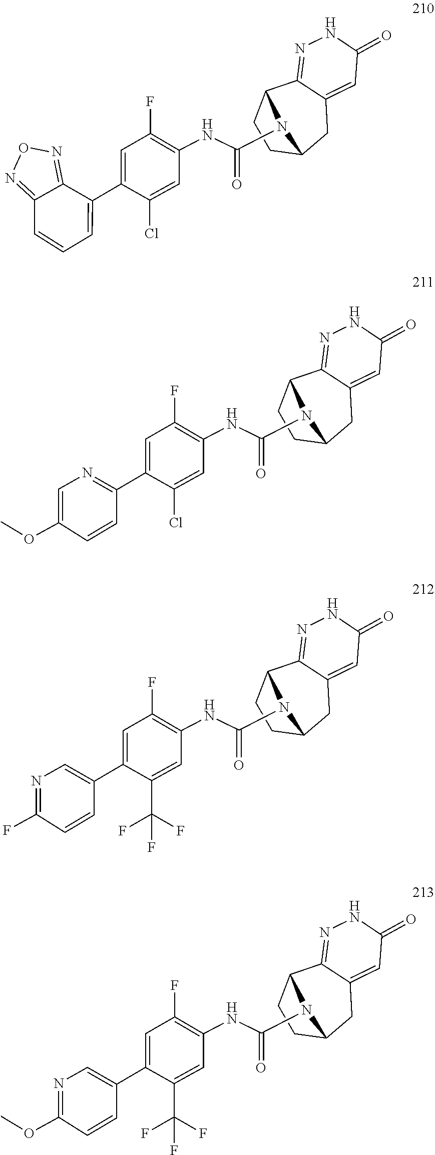 [SMILES: O=C(Nc1cc(Cl)c(-c2cccc3nonc23)cc1F)N1[C@H]2CC[C@@H]1c1n[nH]c(=O)cc1C2 ; COc1ccc(-c2cc(F)c(NC(=O)N3[C@H]4CC[C@@H]3c3n[nH]c(=O)cc3C4)cc2Cl)nc1 ; O=C(Nc1cc(C(F)(F)F)c(-c2ccc(F)nc2)cc1F)N1[C@H]2CC[C@@H]1c1n[nH]c(=O)cc1C2 ; COc1ccc(-c2cc(F)c(NC(=O)N3[C@H]4CC[C@@H]3c3n[nH]c(=O)cc3C4)cc2C(F)(F)F)cn1] 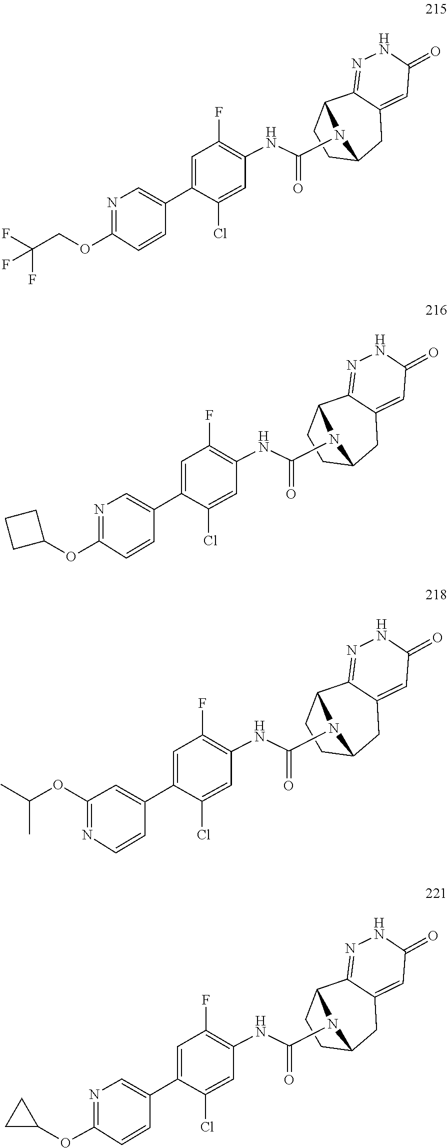 [SMILES: O=C(Nc1cc(Cl)c(-c2ccc(OCC(F)(F)F)nc2)cc1F)N1[C@H]2CC[C@@H]1c1n[nH]c(=O)cc1C2 ; O=C(Nc1cc(Cl)c(-c2ccc(OC3CCC3)nc2)cc1F)N1[C@H]2CC[C@@H]1c1n[nH]c(=O)cc1C2 ; CC(C)Oc1cc(-c2cc(F)c(NC(=O)N3[C@H]4CC[C@@H]3c3n[nH]c(=O)cc3C4)cc2Cl)ccn1 ; O=C(Nc1cc(Cl)c(-c2ccc(OC3CC3)nc2)cc1F)N1[C@H]2CC[C@@H]1c1n[nH]c(=O)cc1C2] 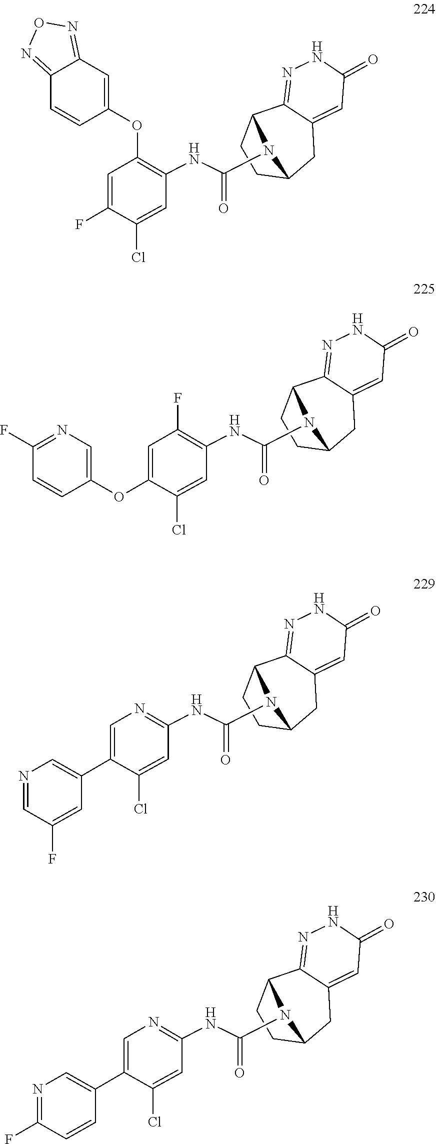 [SMILES: O=C(Nc1cc(Cl)c(F)cc1Oc1ccc2nonc2c1)N1[C@H]2CC[C@@H]1c1n[nH]c(=O)cc1C2 ; O=C(Nc1cc(Cl)c(Oc2ccc(F)nc2)cc1F)N1[C@H]2CC[C@@H]1c1n[nH]c(=O)cc1C2 ; O=C(Nc1cc(Cl)c(-c2cncc(F)c2)cn1)N1[C@H]2CC[C@@H]1c1n[nH]c(=O)cc1C2 ; O=C(Nc1cc(Cl)c(-c2ccc(F)nc2)cn1)N1[C@H]2CC[C@@H]1c1n[nH]c(=O)cc1C2] 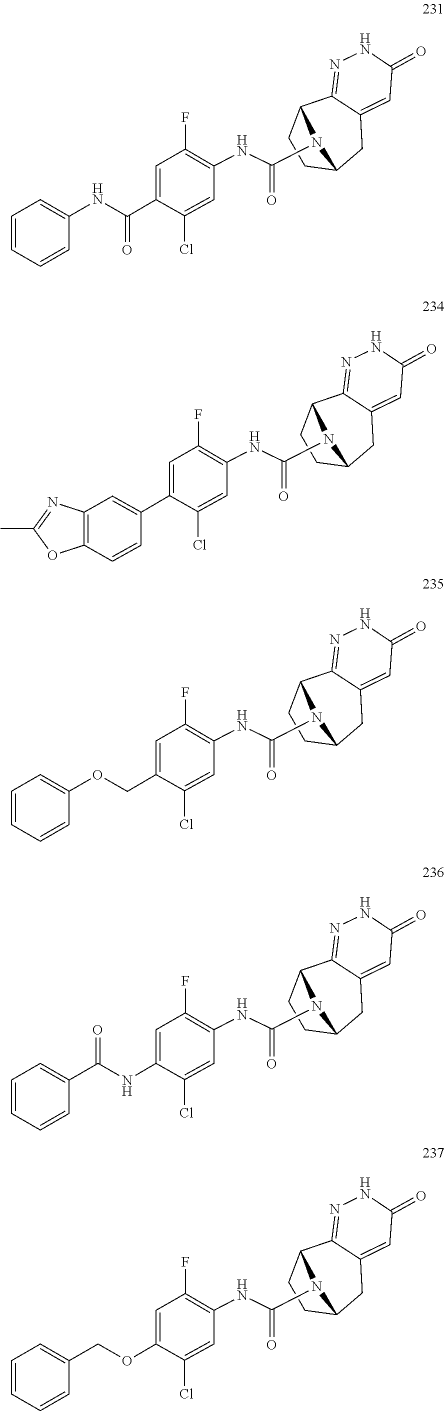 [SMILES: O=C(Nc1ccccc1)c1cc(F)c(NC(=O)N2[C@H]3CC[C@@H]2c2n[nH]c(=O)cc2C3)cc1Cl ; Cc1nc2cc(-c3cc(F)c(NC(=O)N4[C@H]5CC[C@@H]4c4n[nH]c(=O)cc4C5)cc3Cl)ccc2o1 ; O=C(Nc1cc(Cl)c(COc2ccccc2)cc1F)N1[C@H]2CC[C@@H]1c1n[nH]c(=O)cc1C2 ; O=C(Nc1cc(F)c(NC(=O)N2[C@H]3CC[C@@H]2c2n[nH]c(=O)cc2C3)cc1Cl)c1ccccc1 ; O=C(Nc1cc(Cl)c(OCc2ccccc2)cc1F)N1[C@H]2CC[C@@H]1c1n[nH]c(=O)cc1C2] 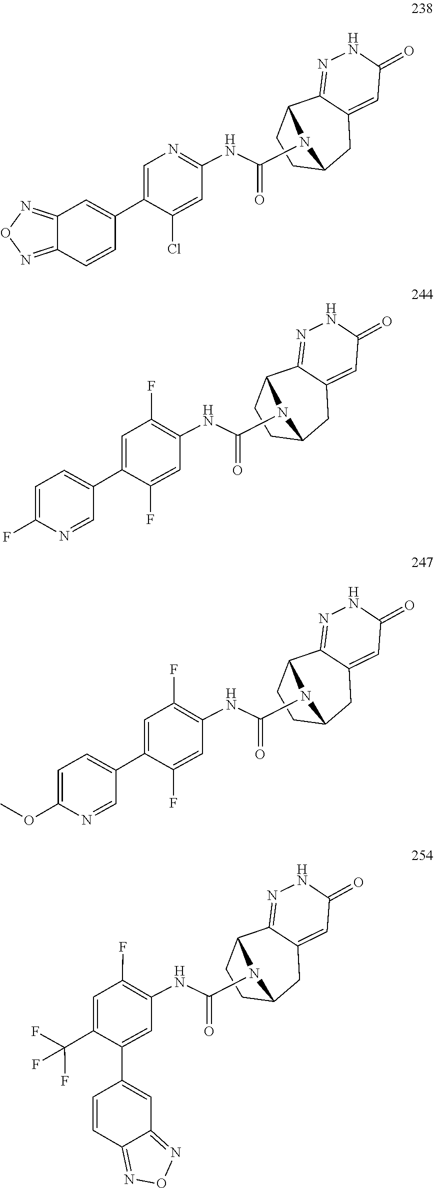 [SMILES: O=C(Nc1cc(Cl)c(-c2ccc3nonc3c2)cn1)N1[C@H]2CC[C@@H]1c1n[nH]c(=O)cc1C2 ; O=C(Nc1cc(F)c(-c2ccc(F)nc2)cc1F)N1[C@H]2CC[C@@H]1c1n[nH]c(=O)cc1C2 ; COc1ccc(-c2cc(F)c(NC(=O)N3[C@H]4CC[C@@H]3c3n[nH]c(=O)cc3C4)cc2F)cn1 ; O=C(Nc1cc(-c2ccc3nonc3c2)c(C(F)(F)F)cc1F)N1[C@H]2CC[C@@H]1c1n[nH]c(=O)cc1C2] 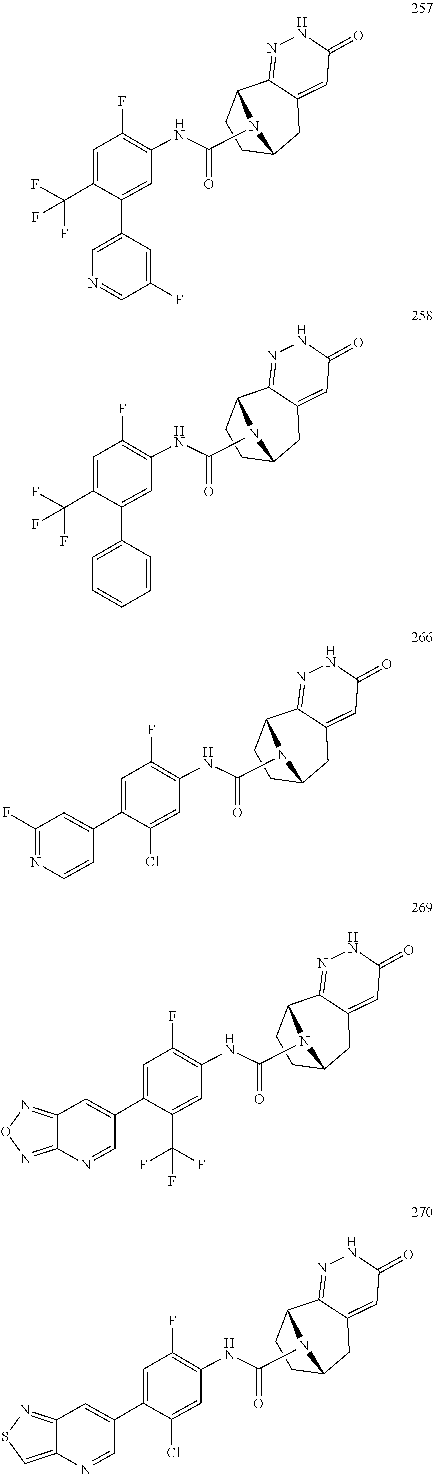 [SMILES: O=C(Nc1cc(-c2cncc(F)c2)c(C(F)(F)F)cc1F)N1[C@H]2CC[C@@H]1c1n[nH]c(=O)cc1C2 ; O=C(Nc1cc(-c2ccccc2)c(C(F)(F)F)cc1F)N1[C@H]2CC[C@@H]1c1n[nH]c(=O)cc1C2 ; O=C(Nc1cc(Cl)c(-c2ccnc(F)c2)cc1F)N1[C@H]2CC[C@@H]1c1n[nH]c(=O)cc1C2 ; O=C(Nc1cc(C(F)(F)F)c(-c2cnc3nonc3c2)cc1F)N1[C@H]2CC[C@@H]1c1n[nH]c(=O)cc1C2 ; O=C(Nc1cc(Cl)c(-c2cnc3csnc3c2)cc1F)N1[C@H]2CC[C@@H]1c1n[nH]c(=O)cc1C2] 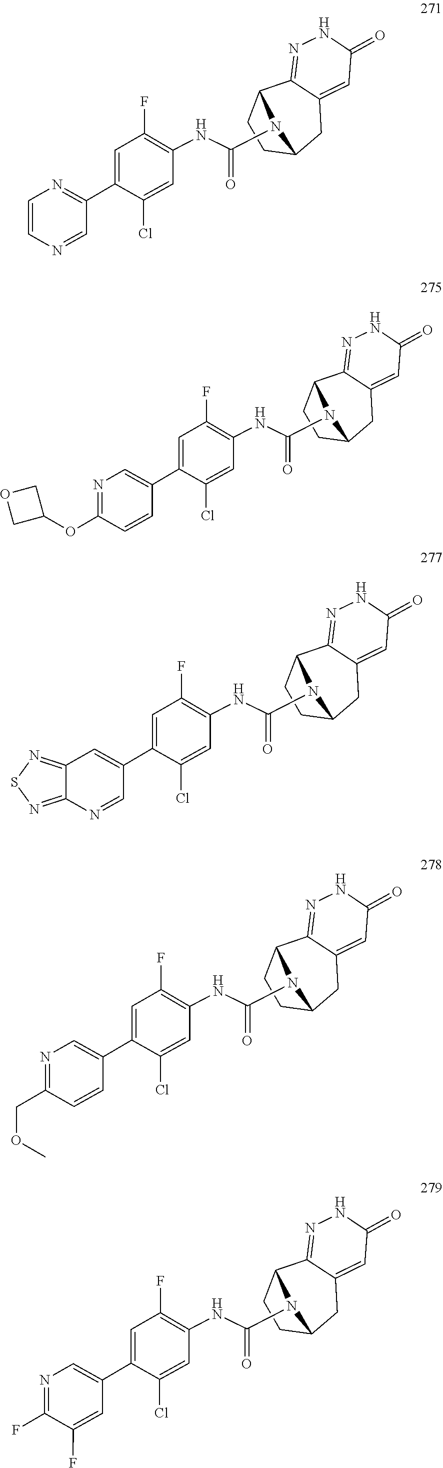 [SMILES: O=C(Nc1cc(Cl)c(-c2cnccn2)cc1F)N1[C@H]2CC[C@@H]1c1n[nH]c(=O)cc1C2 ; O=C(Nc1cc(Cl)c(-c2ccc(OC3COC3)nc2)cc1F)N1[C@H]2CC[C@@H]1c1n[nH]c(=O)cc1C2 ; O=C(Nc1cc(Cl)c(-c2cnc3nsnc3c2)cc1F)N1[C@H]2CC[C@@H]1c1n[nH]c(=O)cc1C2 ; COCc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2cnc(F)c(F)c2)cc1F)N1[C@H]2CC[C@@H]1c1n[nH]c(=O)cc1C2] 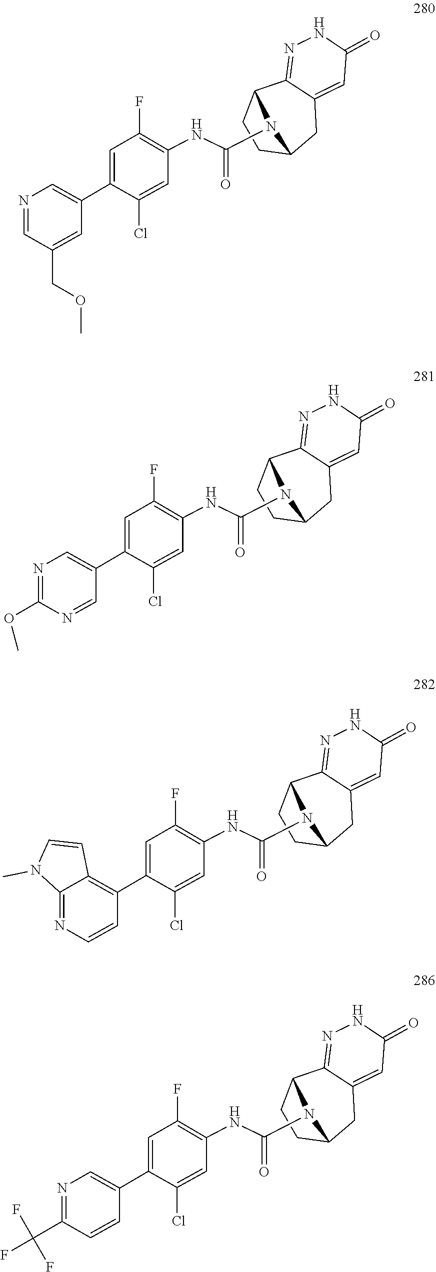 [SMILES: COCc1cncc(-c2cc(F)c(NC(=O)N3[C@H]4CC[C@@H]3c3n[nH]c(=O)cc3C4)cc2Cl)c1 ; COc1ncc(-c2cc(F)c(NC(=O)N3[C@H]4CC[C@@H]3c3n[nH]c(=O)cc3C4)cc2Cl)cn1 ; Cn1ccc2c(-c3cc(F)c(NC(=O)N4[C@H]5CC[C@@H]4c4n[nH]c(=O)cc4C5)cc3Cl)ccnc21 ; O=C(Nc1cc(Cl)c(-c2ccc(C(F)(F)F)nc2)cc1F)N1[C@H]2CC[C@@H]1c1n[nH]c(=O)cc1C2] 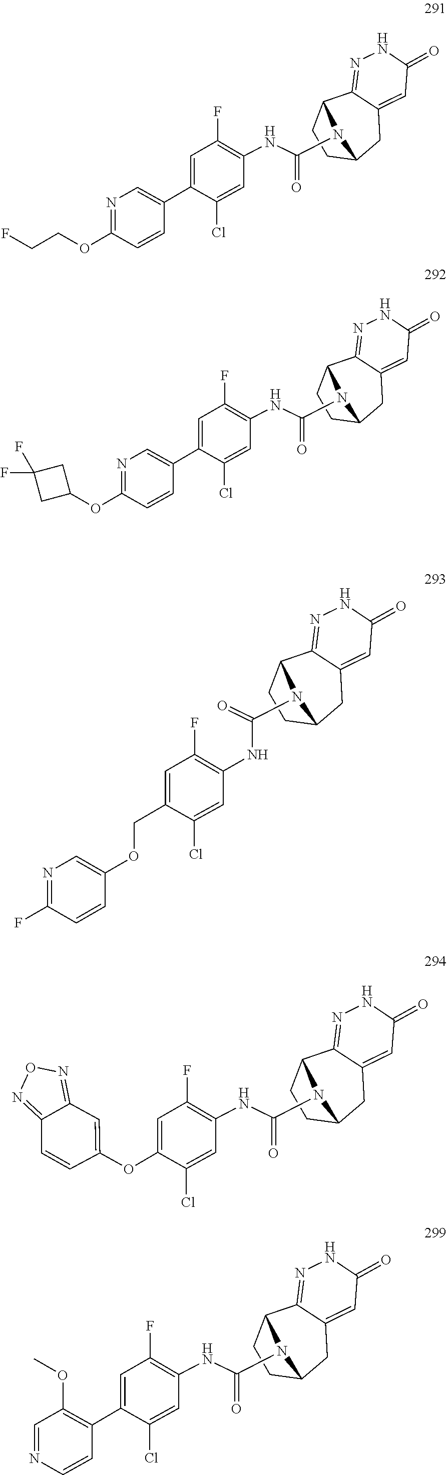 [SMILES: O=C(Nc1cc(Cl)c(-c2ccc(OCCF)nc2)cc1F)N1[C@H]2CC[C@@H]1c1n[nH]c(=O)cc1C2 ; O=C(Nc1cc(Cl)c(-c2ccc(OC3CC(F)(F)C3)nc2)cc1F)N1[C@H]2CC[C@@H]1c1n[nH]c(=O)cc1C2 ; O=C(Nc1cc(Cl)c(COc2ccc(F)nc2)cc1F)N1[C@H]2CC[C@@H]1c1n[nH]c(=O)cc1C2 ; O=C(Nc1cc(Cl)c(Oc2ccc3nonc3c2)cc1F)N1[C@H]2CC[C@@H]1c1n[nH]c(=O)cc1C2 ; COc1cnccc1-c1cc(F)c(NC(=O)N2[C@H]3CC[C@@H]2c2n[nH]c(=O)cc2C3)cc1Cl] 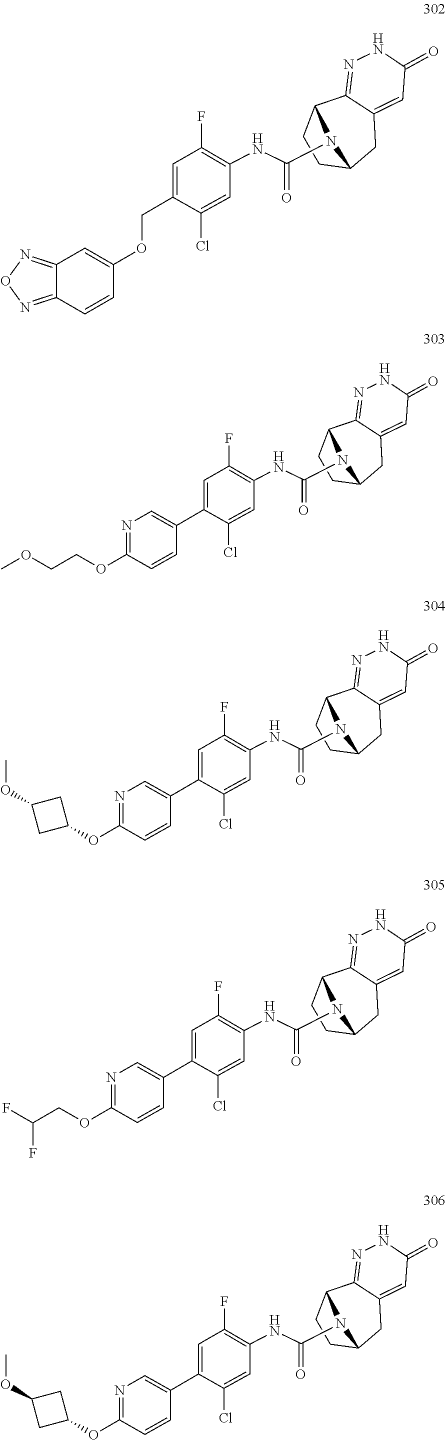 [SMILES: O=C(Nc1cc(Cl)c(COc2ccc3nonc3c2)cc1F)N1[C@H]2CC[C@@H]1c1n[nH]c(=O)cc1C2 ; COCCOc1ccc(-c2cc(F)c(NC(=O)N3[C@H]4CC[C@@H]3c3n[nH]c(=O)cc3C4)cc2Cl)cn1 ; CO[C@H]1C[C@@H](Oc2ccc(-c3cc(F)c(NC(=O)N4[C@H]5CC[C@@H]4c4n[nH]c(=O)cc4C5)cc3Cl)cn2)C1 ; O=C(Nc1cc(Cl)c(-c2ccc(OCC(F)F)nc2)cc1F)N1[C@H]2CC[C@@H]1c1n[nH]c(=O)cc1C2 ; CO[C@H]1C[C@H](Oc2ccc(-c3cc(F)c(NC(=O)N4[C@H]5CC[C@@H]4c4n[nH]c(=O)cc4C5)cc3Cl)cn2)C1] 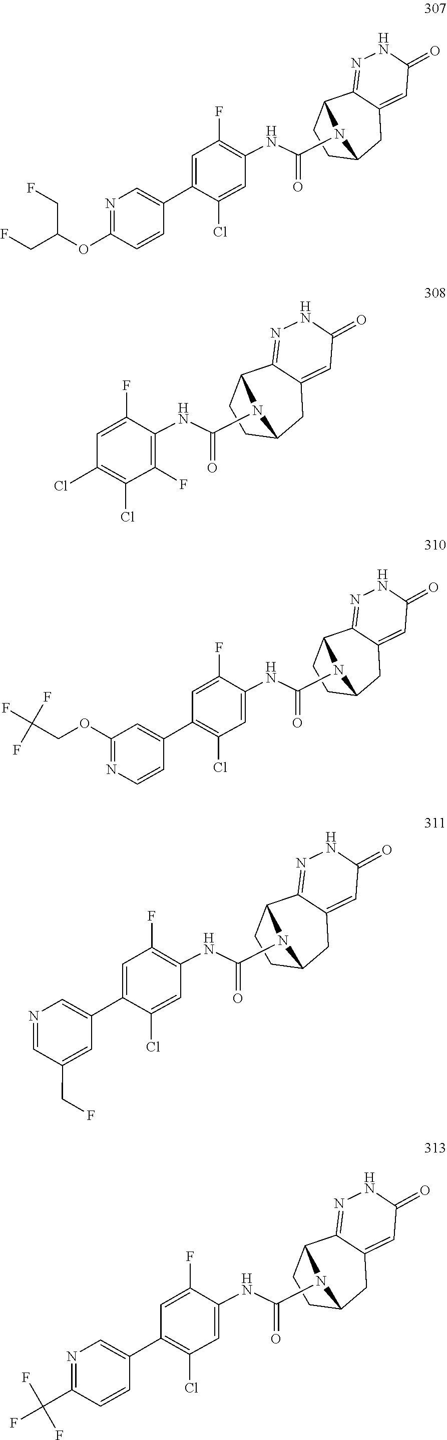 [SMILES: O=C(Nc1cc(Cl)c(-c2ccc(OC(CF)CF)nc2)cc1F)N1[C@H]2CC[C@@H]1c1n[nH]c(=O)cc1C2 ; O=C(Nc1c(F)cc(Cl)c(Cl)c1F)N1[C@H]2CC[C@@H]1c1n[nH]c(=O)cc1C2 ; O=C(Nc1cc(Cl)c(-c2ccnc(OCC(F)(F)F)c2)cc1F)N1[C@H]2CC[C@@H]1c1n[nH]c(=O)cc1C2 ; O=C(Nc1cc(Cl)c(-c2cncc(CF)c2)cc1F)N1[C@H]2CC[C@@H]1c1n[nH]c(=O)cc1C2 ; O=C(Nc1cc(Cl)c(-c2ccc(C(F)(F)F)nc2)cc1F)N1[C@H]2CC[C@@H]1c1n[nH]c(=O)cc1C2] 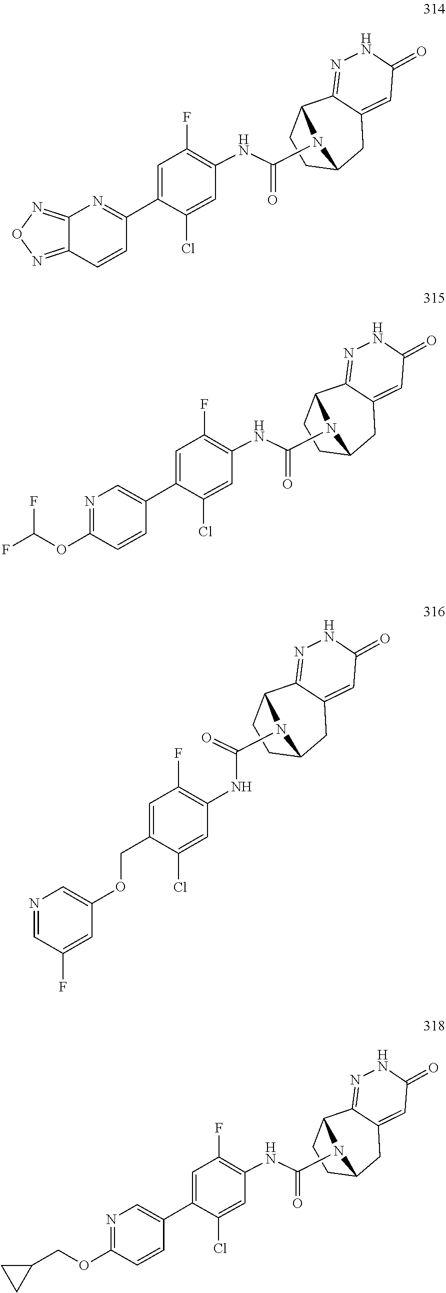 [SMILES: O=C(Nc1cc(Cl)c(-c2ccc3nonc3n2)cc1F)N1[C@H]2CC[C@@H]1c1n[nH]c(=O)cc1C2 ; O=C(Nc1cc(Cl)c(-c2ccc(OC(F)F)nc2)cc1F)N1[C@H]2CC[C@@H]1c1n[nH]c(=O)cc1C2 ; O=C(Nc1cc(Cl)c(COc2cncc(F)c2)cc1F)N1[C@H]2CC[C@@H]1c1n[nH]c(=O)cc1C2 ; O=C(Nc1cc(Cl)c(-c2ccc(OCC3CC3)nc2)cc1F)N1[C@H]2CC[C@@H]1c1n[nH]c(=O)cc1C2] 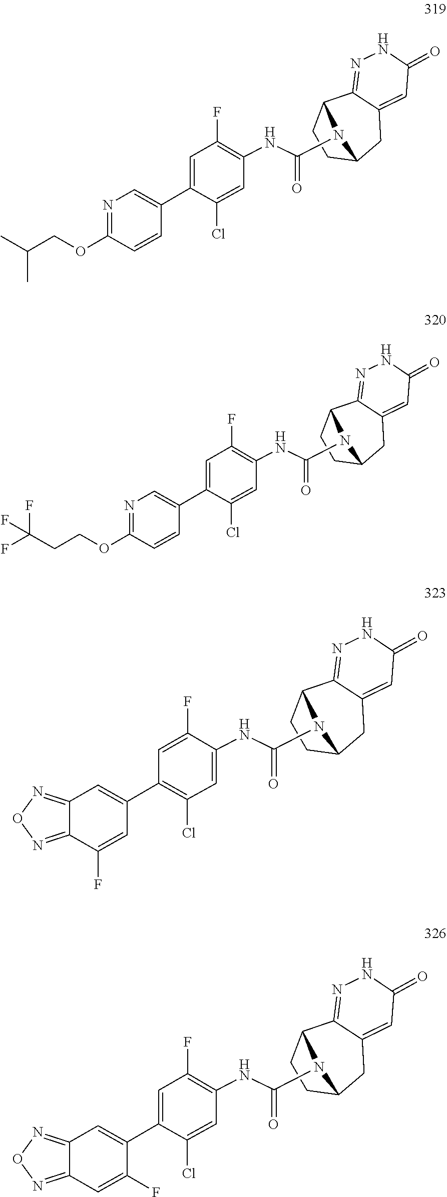 [SMILES: CC(C)COc1ccc(-c2cc(F)c(NC(=O)N3[C@H]4CC[C@@H]3c3n[nH]c(=O)cc3C4)cc2Cl)cn1 ; O=C(Nc1cc(Cl)c(-c2ccc(OCCC(F)(F)F)nc2)cc1F)N1[C@H]2CC[C@@H]1c1n[nH]c(=O)cc1C2 ; O=C(Nc1cc(Cl)c(-c2cc(F)c3nonc3c2)cc1F)N1[C@H]2CC[C@@H]1c1n[nH]c(=O)cc1C2 ; O=C(Nc1cc(Cl)c(-c2cc3nonc3cc2F)cc1F)N1[C@H]2CC[C@@H]1c1n[nH]c(=O)cc1C2] 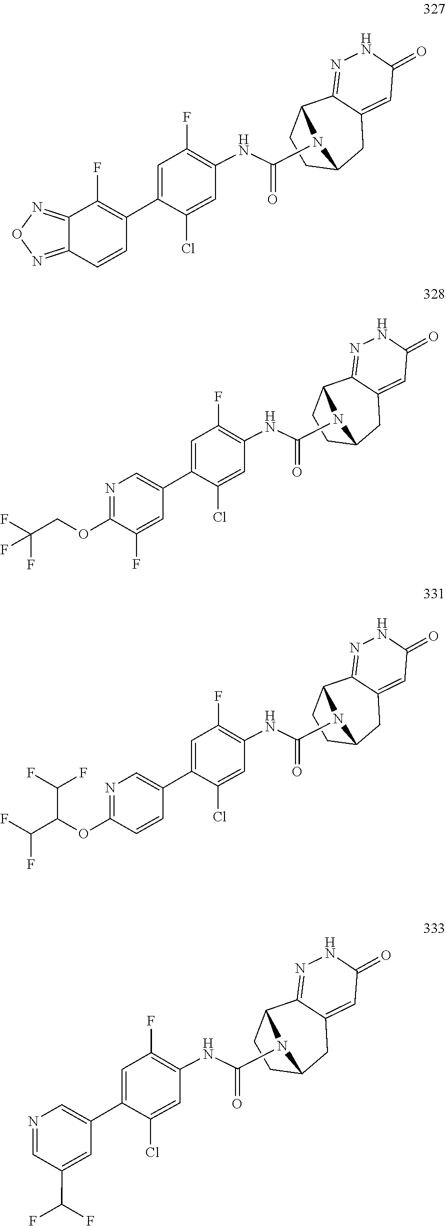 [SMILES: O=C(Nc1cc(Cl)c(-c2ccc3nonc3c2F)cc1F)N1[C@H]2CC[C@@H]1c1n[nH]c(=O)cc1C2 ; O=C(Nc1cc(Cl)c(-c2cnc(OCC(F)(F)F)c(F)c2)cc1F)N1[C@H]2CC[C@@H]1c1n[nH]c(=O)cc1C2 ; O=C(Nc1cc(Cl)c(-c2ccc(OC(C(F)F)C(F)F)nc2)cc1F)N1[C@H]2CC[C@@H]1c1n[nH]c(=O)cc1C2 ; O=C(Nc1cc(Cl)c(-c2cncc(C(F)F)c2)cc1F)N1[C@H]2CC[C@@H]1c1n[nH]c(=O)cc1C2] 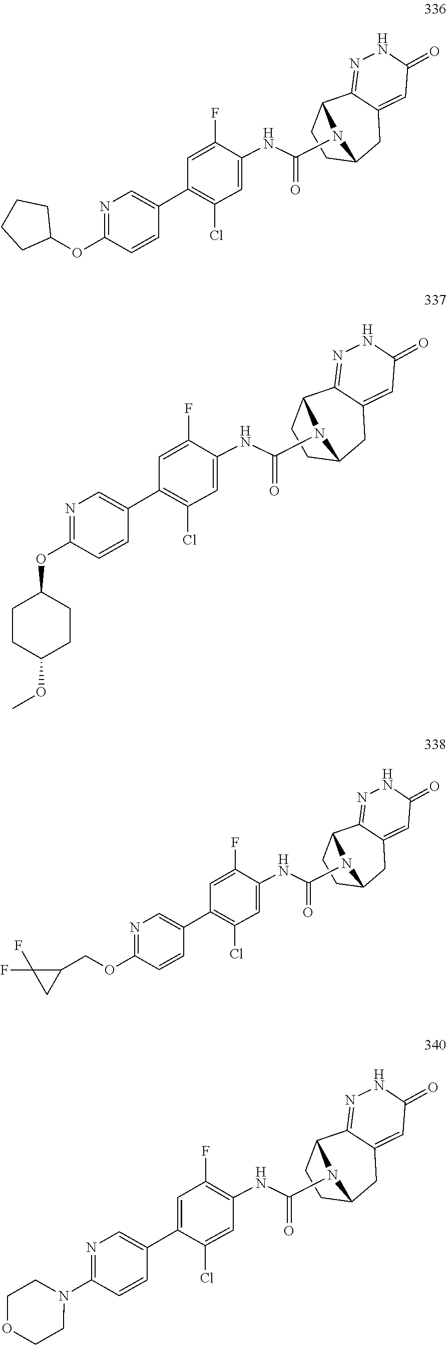 [SMILES: O=C(Nc1cc(Cl)c(-c2ccc(OC3CCCC3)nc2)cc1F)N1[C@H]2CC[C@@H]1c1n[nH]c(=O)cc1C2 ; CO[C@H]1CC[C@H](Oc2ccc(-c3cc(F)c(NC(=O)N4[C@H]5CC[C@@H]4c4n[nH]c(=O)cc4C5)cc3Cl)cn2)CC1 ; O=C(Nc1cc(Cl)c(-c2ccc(OCC3CC3(F)F)nc2)cc1F)N1[C@H]2CC[C@@H]1c1n[nH]c(=O)cc1C2 ; O=C(Nc1cc(Cl)c(-c2ccc(N3CCOCC3)nc2)cc1F)N1[C@H]2CC[C@@H]1c1n[nH]c(=O)cc1C2] 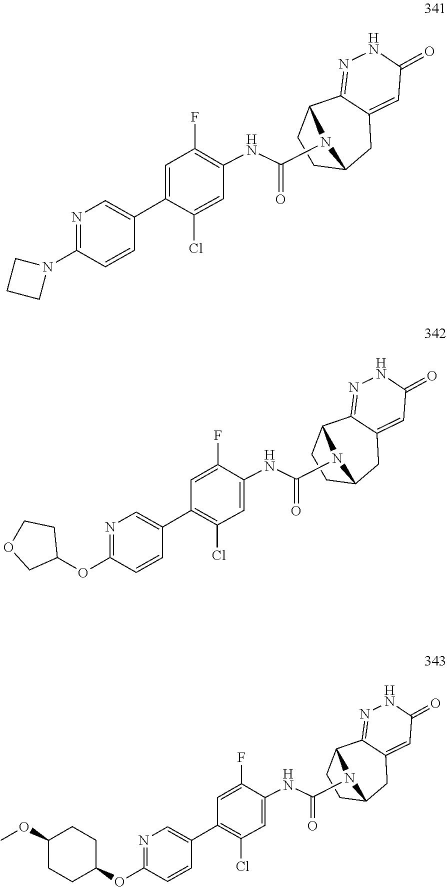 [SMILES: O=C(Nc1cc(Cl)c(-c2ccc(N3CCC3)nc2)cc1F)N1[C@H]2CC[C@@H]1c1n[nH]c(=O)cc1C2 ; O=C(Nc1cc(Cl)c(-c2ccc(OC3CCOC3)nc2)cc1F)N1[C@H]2CC[C@@H]1c1n[nH]c(=O)cc1C2 ; CO[C@H]1CC[C@@H](Oc2ccc(-c3cc(F)c(NC(=O)N4[C@H]5CC[C@@H]4c4n[nH]c(=O)cc4C5)cc3Cl)cn2)CC1]
and enantiomers thereof, and mixtures of enantiomers thereof, including racemic mixtures, and pharmaceutically acceptable salts and solvates thereof.

27. A compound of claim 1, having the formula (If), or a pharmaceutically acceptable salt or solvate thereof, 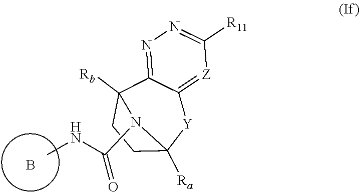
wherein:
R₁₁ is selected from H, alkyl, haloalkyl, halo, OH and O-alkyl; and
B, Y, Z, Rₐ and R_{b} are as defined in claim 1.

28. A compound of formula (Ij) or a pharmaceutically acceptable salt or solvate thereof, 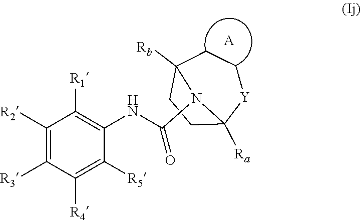
wherein ring A is selected from: 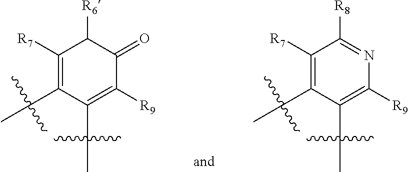
Y is CR₁₀R_{10′}, wherein R₁₀ and R_{10′} are each independently selected from H, F, alkyl, and haloalkyl;
Rₐ and R_{b} are each independently selected from H and alkyl;
R_{1′}, R_{2′}, R_{4′} and R_{5′} are each independently selected from H, CN, alkyl, alkoxy, haloalkyl, OH and halo;
R_{3′} is a pyridinyl group substituted by one or more substituents selected from CN, haloalkyl, haloalkoxy, NHCO-alkyl, NR₁₃R_{13′}, SO₂-alkyl, O—(CH₂)_{q}-heterocycloalkyl, alkoxy-alkoxy, alkylamino-alkoxy, dialkylamino-alkoxy, alkoxy-alkyl and O—(CH₂)ₚ-cycloalkyl, wherein said cycloalkyl group is optionally substituted by one or more halo, alkyl or alkoxy groups, and wherein said pyridinyl is optionally further substituted by one or more substituents selected from halo, alkyl and alkoxy;
R_{6′} is H or alkyl, more preferably H;
R₇, R₈ and R₉ are each independently selected from H, halo and alkyl;
R₁₃ and R_{13′} are each independently selected from H, alkyl, and alkoxy-alkyl; and
p and q are each independently 0 to 3.

29. A compound according to claim 28, wherein:
R_{1′} and R_{4′} are selected from halo and haloalkyl;
R_{2′} and R_{5′} are H;
R_{3′} is: 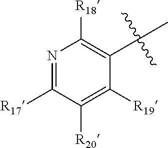
wherein:
R_{17′} is selected from CN, haloalkyl, haloalkoxy, NHCO-alkyl, NR₁₃R_{13′}, SO₂-alkyl, O—(CH₂)_{q}-heterocycloalkyl, alkoxy-alkoxy, alkylamino-alkoxy, dialkylamino-alkoxy, alkoxy-alkyl and O—(CH₂)ₚ-cycloalkyl, wherein said cycloalkyl group is optionally substituted by one or more halo, alkyl or alkoxy groups;
R_{13′} is selected from H and halo;
R_{19′} is selected from H, alkoxy and alkoxy-alkyl; and
R_{20′} is selected from H and halo.

30. A compound according to claim 29 wherein:
ring A is selected from: 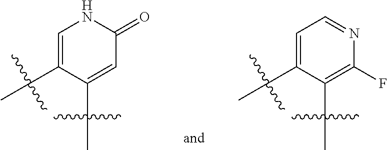 [SMILES: *c1c[nH]c(=O)cc1* ; *c1ccnc(F)c1*]
Y is CH₂;
Rₐ and R_{b} are both H;
R_{1′} is F and R_{4′} is Cl; and
R_{17′} is selected from haloalkyl, haloalkoxy, alkylamino-alkoxy, dialkylamino-alkoxy, O—(CH₂)_{q}-heterocycloalkyl, O—(CH₂)ₚ-cycloalkyl, wherein said cycloalkyl group is optionally substituted by one or more alkoxy groups;
p is 0 or 1;
q is 0 or 1; and
R_{18′}, R_{19′} and R_{20′} are all H.

31. A compound selected from the following: 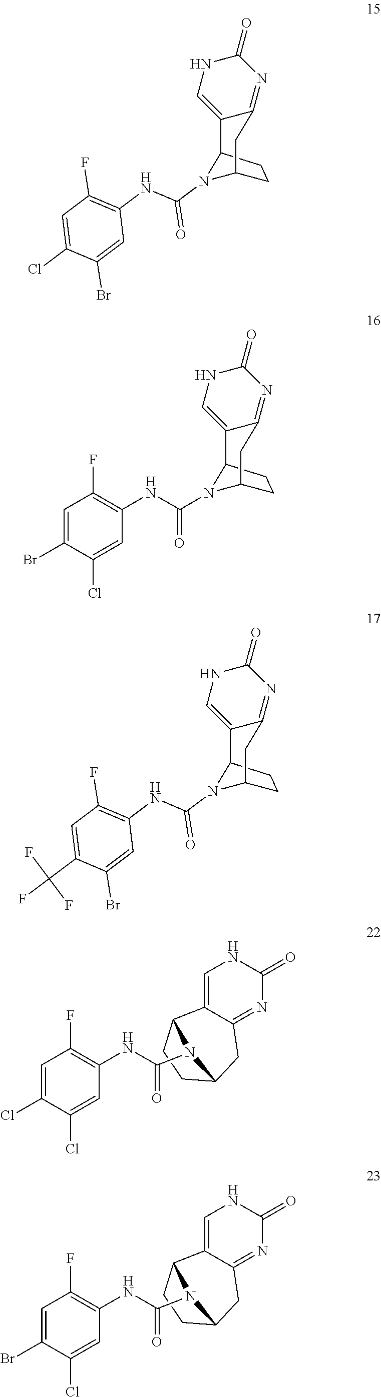 [SMILES: O=C(Nc1cc(Br)c(Cl)cc1F)N1C2CCC1c1c[nH]c(=O)nc1C2 ; O=C(Nc1cc(Cl)c(Br)cc1F)N1C2CCC1c1c[nH]c(=O)nc1C2 ; O=C(Nc1cc(Br)c(C(F)(F)F)cc1F)N1C2CCC1c1c[nH]c(=O)nc1C2 ; O=C(Nc1cc(Cl)c(Cl)cc1F)N1[C@H]2CC[C@@H]1c1c[nH]c(=O)nc1C2 ; O=C(Nc1cc(Cl)c(Br)cc1F)N1[C@H]2CC[C@@H]1c1c[nH]c(=O)nc1C2] 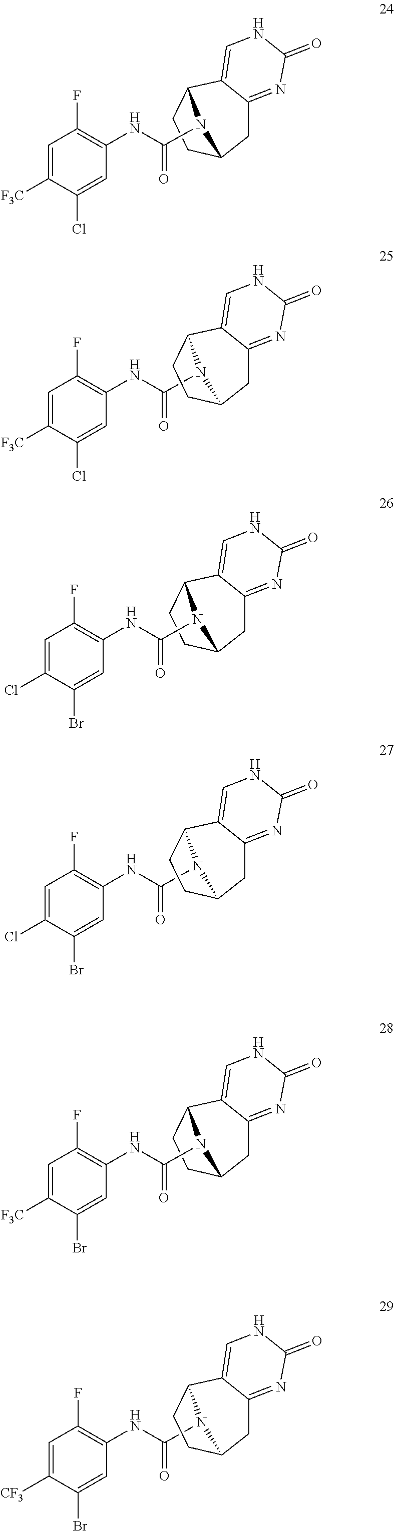 [SMILES: O=C(Nc1cc(Cl)c(C(F)(F)F)cc1F)N1[C@H]2CC[C@@H]1c1c[nH]c(=O)nc1C2 ; O=C(Nc1cc(Cl)c(C(F)(F)F)cc1F)N1[C@@H]2CC[C@H]1c1c[nH]c(=O)nc1C2 ; O=C(Nc1cc(Br)c(Cl)cc1F)N1[C@H]2CC[C@@H]1c1c[nH]c(=O)nc1C2 ; O=C(Nc1cc(Br)c(Cl)cc1F)N1[C@@H]2CC[C@H]1c1c[nH]c(=O)nc1C2 ; O=C(Nc1cc(Br)c(C(F)(F)F)cc1F)N1[C@H]2CC[C@@H]1c1c[nH]c(=O)nc1C2 ; O=C(Nc1cc(Br)c(C(F)(F)F)cc1F)N1[C@@H]2CC[C@H]1c1c[nH]c(=O)nc1C2] 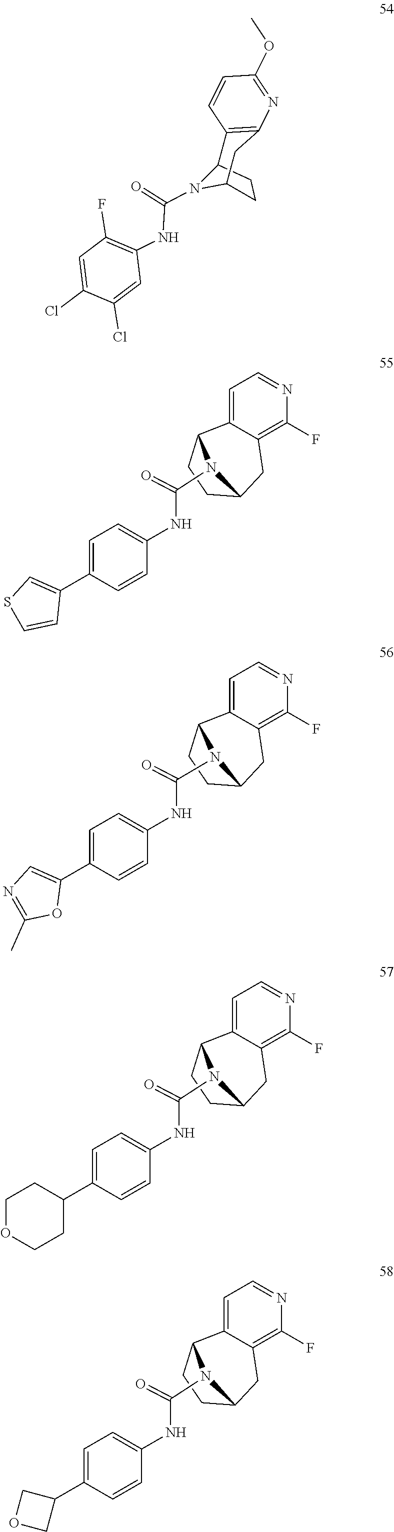 [SMILES: COc1ccc2c(n1)CC1CCC2N1C(=O)Nc1cc(Cl)c(Cl)cc1F ; O=C(Nc1ccc(-c2ccsc2)cc1)N1[C@H]2CC[C@@H]1c1ccnc(F)c1C2 ; Cc1ncc(-c2ccc(NC(=O)N3[C@H]4CC[C@@H]3c3ccnc(F)c3C4)cc2)o1 ; O=C(Nc1ccc(C2CCOCC2)cc1)N1[C@H]2CC[C@@H]1c1ccnc(F)c1C2 ; O=C(Nc1ccc(C2COC2)cc1)N1[C@H]2CC[C@@H]1c1ccnc(F)c1C2] 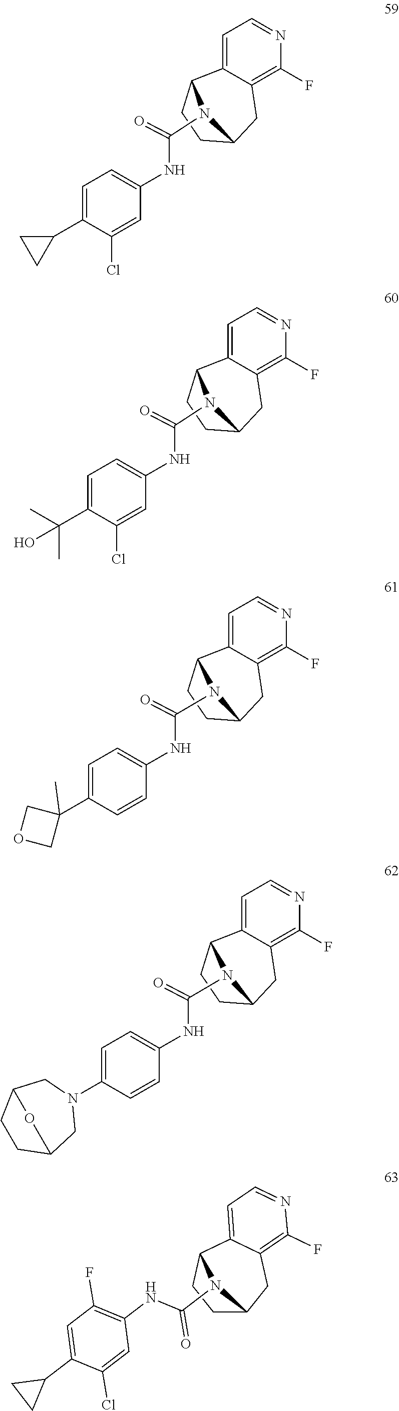 [SMILES: O=C(Nc1ccc(C2CC2)c(Cl)c1)N1[C@H]2CC[C@@H]1c1ccnc(F)c1C2 ; CC(C)(O)c1ccc(NC(=O)N2[C@H]3CC[C@@H]2c2ccnc(F)c2C3)cc1Cl ; CC1(c2ccc(NC(=O)N3[C@H]4CC[C@@H]3c3ccnc(F)c3C4)cc2)COC1 ; O=C(Nc1ccc(N2CC3CCC(C2)O3)cc1)N1[C@H]2CC[C@@H]1c1ccnc(F)c1C2 ; O=C(Nc1cc(Cl)c(C2CC2)cc1F)N1[C@H]2CC[C@@H]1c1ccnc(F)c1C2] 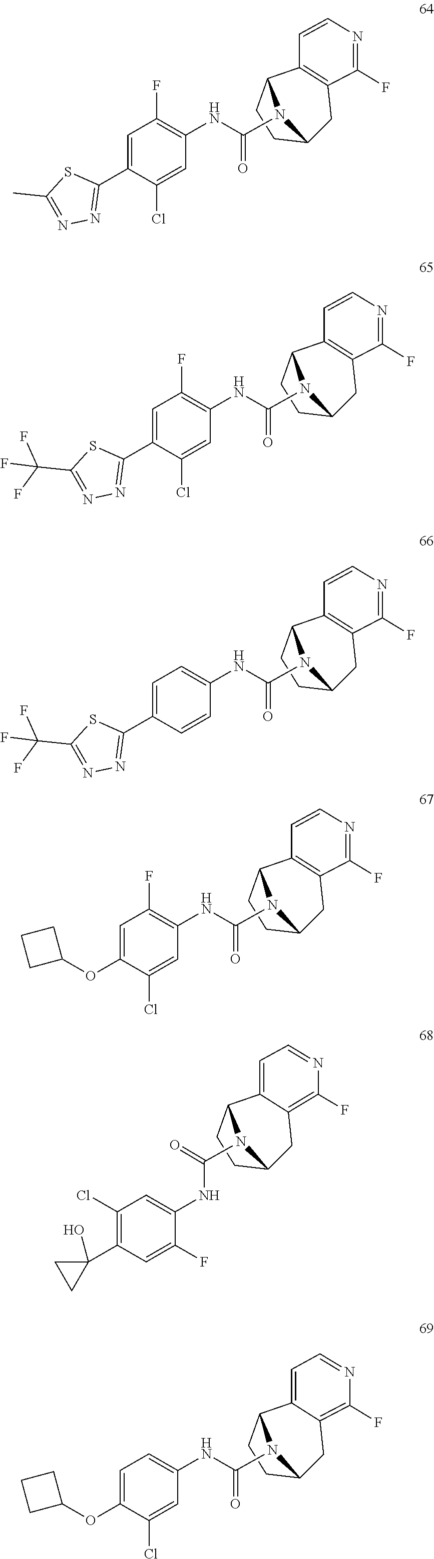 [SMILES: Cc1nnc(-c2cc(F)c(NC(=O)N3[C@H]4CC[C@@H]3c3ccnc(F)c3C4)cc2Cl)s1 ; O=C(Nc1cc(Cl)c(-c2nnc(C(F)(F)F)s2)cc1F)N1[C@H]2CC[C@@H]1c1ccnc(F)c1C2 ; O=C(Nc1ccc(-c2nnc(C(F)(F)F)s2)cc1)N1[C@H]2CC[C@@H]1c1ccnc(F)c1C2 ; O=C(Nc1cc(Cl)c(OC2CCC2)cc1F)N1[C@H]2CC[C@@H]1c1ccnc(F)c1C2 ; O=C(Nc1cc(Cl)c(C2(O)CC2)cc1F)N1[C@H]2CC[C@@H]1c1ccnc(F)c1C2 ; O=C(Nc1ccc(OC2CCC2)c(Cl)c1)N1[C@H]2CC[C@@H]1c1ccnc(F)c1C2] 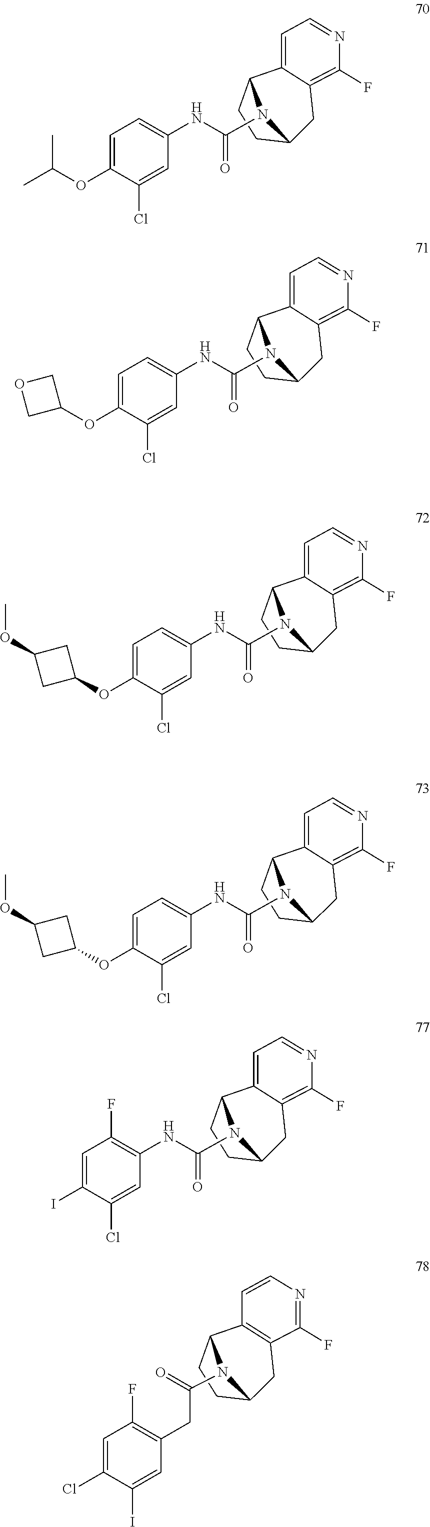 [SMILES: CC(C)Oc1ccc(NC(=O)N2[C@H]3CC[C@@H]2c2ccnc(F)c2C3)cc1Cl ; O=C(Nc1ccc(OC2COC2)c(Cl)c1)N1[C@H]2CC[C@@H]1c1ccnc(F)c1C2 ; CO[C@H]1C[C@@H](Oc2ccc(NC(=O)N3[C@H]4CC[C@@H]3c3ccnc(F)c3C4)cc2Cl)C1 ; CO[C@H]1C[C@H](Oc2ccc(NC(=O)N3[C@H]4CC[C@@H]3c3ccnc(F)c3C4)cc2Cl)C1 ; O=C(Nc1cc(Cl)c(I)cc1F)N1[C@H]2CC[C@@H]1c1ccnc(F)c1C2 ; O=C(Cc1cc(I)c(Cl)cc1F)N1[C@H]2CC[C@@H]1c1ccnc(F)c1C2] 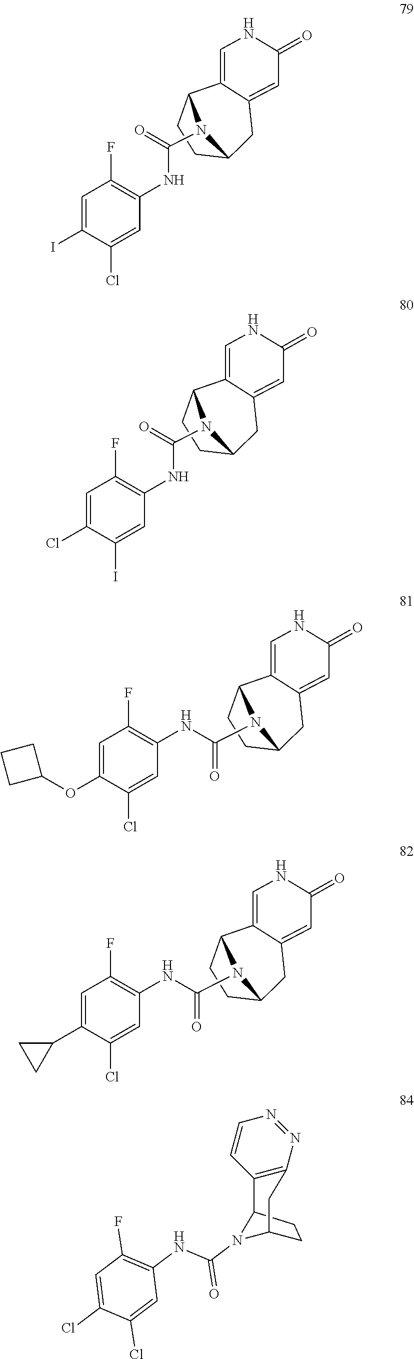 [SMILES: O=C(Nc1cc(Cl)c(I)cc1F)N1[C@H]2CC[C@@H]1c1c[nH]c(=O)cc1C2 ; O=C(Nc1cc(I)c(Cl)cc1F)N1[C@H]2CC[C@@H]1c1c[nH]c(=O)cc1C2 ; O=C(Nc1cc(Cl)c(OC2CCC2)cc1F)N1[C@H]2CC[C@@H]1c1c[nH]c(=O)cc1C2 ; O=C(Nc1cc(Cl)c(C2CC2)cc1F)N1[C@H]2CC[C@@H]1c1c[nH]c(=O)cc1C2 ; O=C(Nc1cc(Cl)c(Cl)cc1F)N1C2CCC1c1ccnnc1C2] 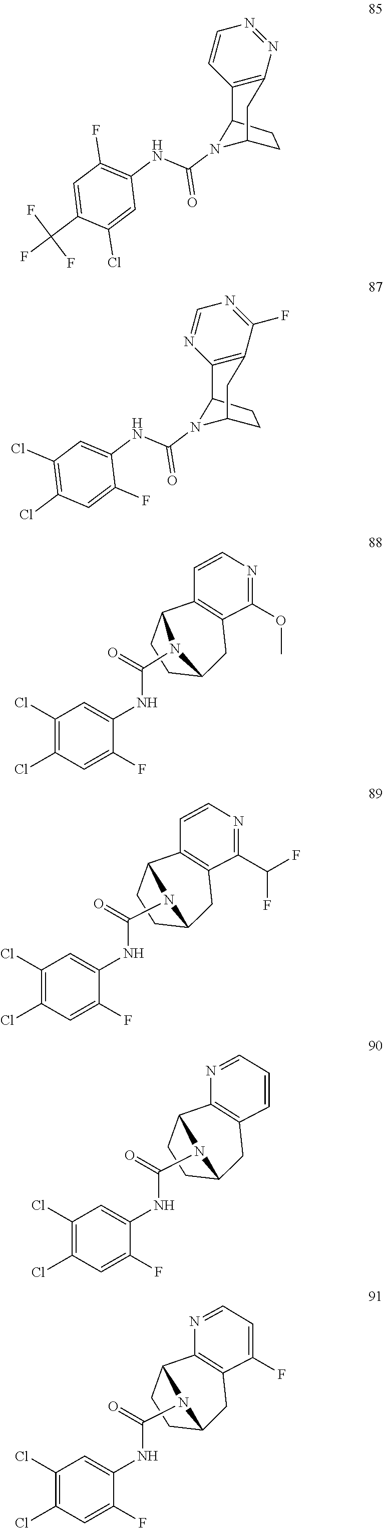 [SMILES: O=C(Nc1cc(Cl)c(C(F)(F)F)cc1F)N1C2CCC1c1ccnnc1C2 ; O=C(Nc1cc(Cl)c(Cl)cc1F)N1C2CCC1c1ncnc(F)c1C2 ; COc1nccc2c1C[C@@H]1CC[C@H]2N1C(=O)Nc1cc(Cl)c(Cl)cc1F ; O=C(Nc1cc(Cl)c(Cl)cc1F)N1[C@H]2CC[C@@H]1c1ccnc(C(F)F)c1C2 ; O=C(Nc1cc(Cl)c(Cl)cc1F)N1[C@H]2CC[C@@H]1c1ncccc1C2 ; O=C(Nc1cc(Cl)c(Cl)cc1F)N1[C@H]2CC[C@@H]1c1nccc(F)c1C2] 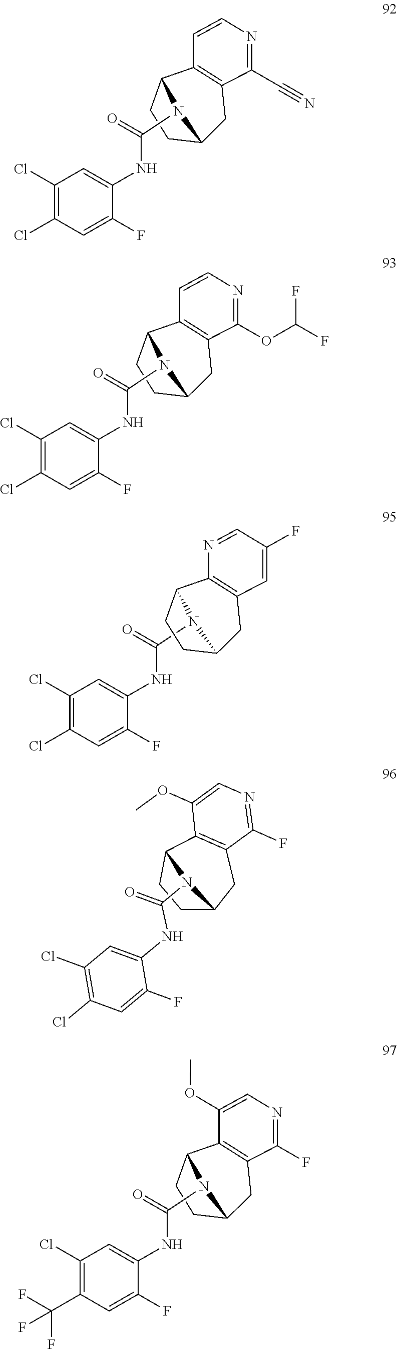 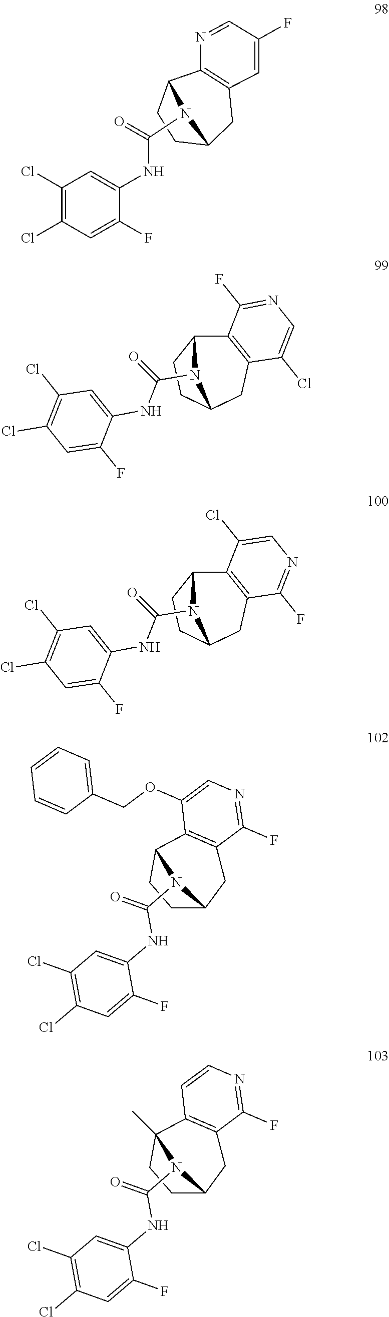 [SMILES: O=C(Nc1cc(Cl)c(Cl)cc1F)N1[C@H]2CC[C@@H]1c1ncc(F)cc1C2 ; O=C(Nc1cc(Cl)c(Cl)cc1F)N1[C@H]2CC[C@@H]1c1c(F)ncc(Cl)c1C2 ; O=C(Nc1cc(Cl)c(Cl)cc1F)N1[C@H]2CC[C@@H]1c1c(Cl)cnc(F)c1C2 ; O=C(Nc1cc(Cl)c(Cl)cc1F)N1[C@H]2CC[C@@H]1c1c(OCc3ccccc3)cnc(F)c1C2 ; C[C@@]12CC[C@@H](Cc3c1ccnc3F)N2C(=O)Nc1cc(Cl)c(Cl)cc1F] 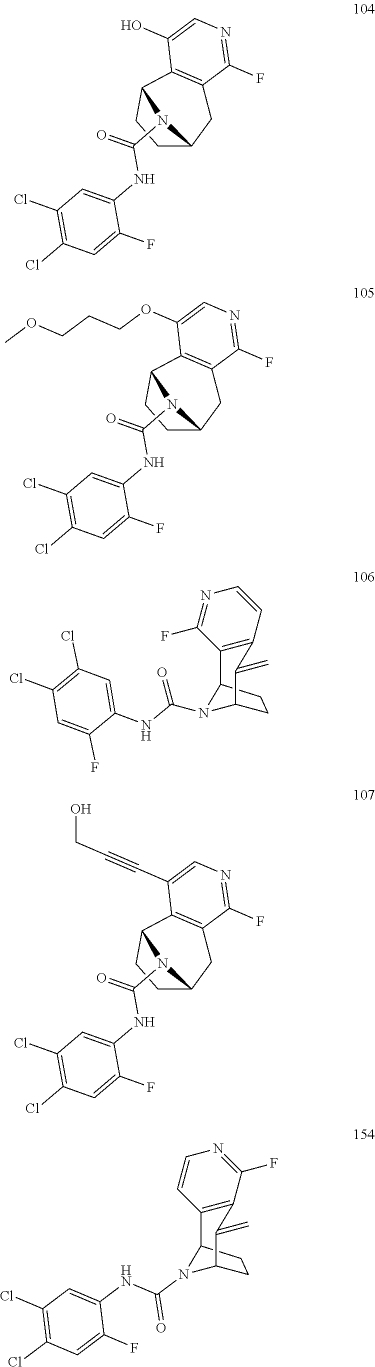 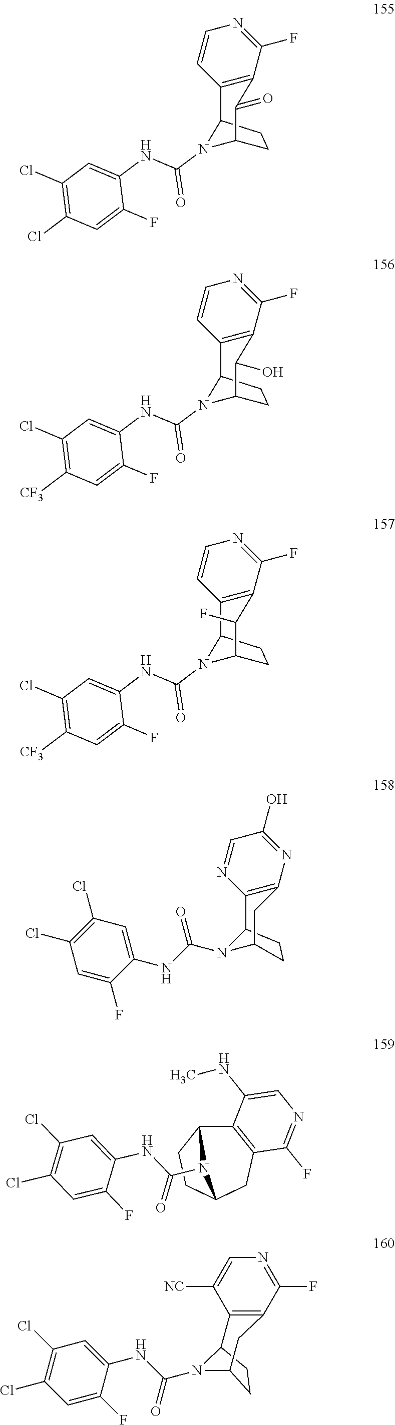 [SMILES: O=C1c2c(ccnc2F)C2CCC1N2C(=O)Nc1cc(Cl)c(Cl)cc1F ; O=C(Nc1cc(Cl)c(C(F)(F)F)cc1F)N1C2CCC1C(O)c1c2ccnc1F ; O=C(Nc1cc(Cl)c(C(F)(F)F)cc1F)N1C2CCC1C(F)c1c2ccnc1F ; O=C(Nc1cc(Cl)c(Cl)cc1F)N1C2CCC1c1ncc(O)nc1C2 ; CNc1cnc(F)c2c1[C@H]1CC[C@@H](C2)N1C(=O)Nc1cc(Cl)c(Cl)cc1F ; N#Cc1cnc(F)c2c1C1CCC(C2)N1C(=O)Nc1cc(Cl)c(Cl)cc1F] 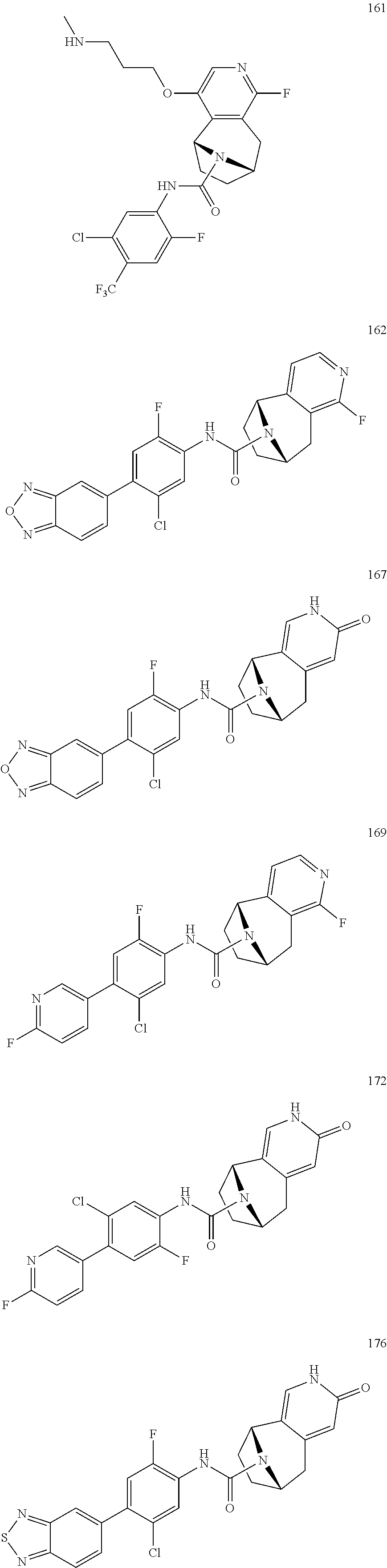 [SMILES: CNCCCOc1cnc(F)c2c1[C@H]1CC[C@@H](C2)N1C(=O)Nc1cc(Cl)c(C(F)(F)F)cc1F ; O=C(Nc1cc(Cl)c(-c2ccc3nonc3c2)cc1F)N1[C@H]2CC[C@@H]1c1ccnc(F)c1C2 ; O=C(Nc1cc(Cl)c(-c2ccc3nonc3c2)cc1F)N1[C@H]2CC[C@@H]1c1c[nH]c(=O)cc1C2 ; O=C(Nc1cc(Cl)c(-c2ccc(F)nc2)cc1F)N1[C@H]2CC[C@@H]1c1ccnc(F)c1C2 ; O=C(Nc1cc(Cl)c(-c2ccc(F)nc2)cc1F)N1[C@H]2CC[C@@H]1c1c[nH]c(=O)cc1C2 ; O=C(Nc1cc(Cl)c(-c2ccc3nsnc3c2)cc1F)N1[C@H]2CC[C@@H]1c1c[nH]c(=O)cc1C2] 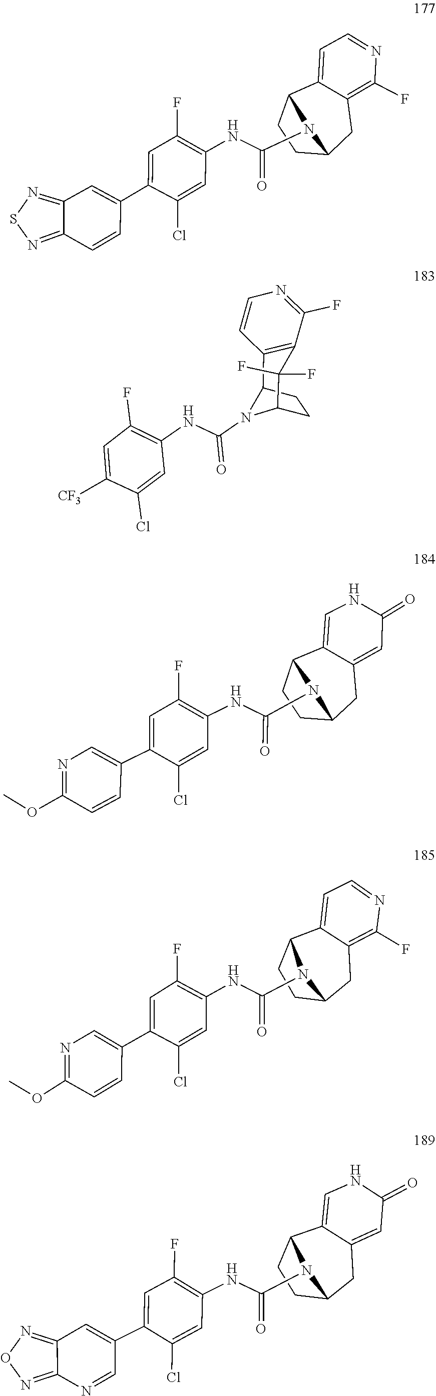 [SMILES: O=C(Nc1cc(Cl)c(-c2ccc3nsnc3c2)cc1F)N1[C@H]2CC[C@@H]1c1ccnc(F)c1C2 ; O=C(Nc1cc(Cl)c(C(F)(F)F)cc1F)N1C2CCC1C(F)(F)c1c2ccnc1F ; COc1ccc(-c2cc(F)c(NC(=O)N3[C@H]4CC[C@@H]3c3c[nH]c(=O)cc3C4)cc2Cl)cn1 ; COc1ccc(-c2cc(F)c(NC(=O)N3[C@H]4CC[C@@H]3c3ccnc(F)c3C4)cc2Cl)cn1 ; O=C(Nc1cc(Cl)c(-c2cnc3nonc3c2)cc1F)N1[C@H]2CC[C@@H]1c1c[nH]c(=O)cc1C2] 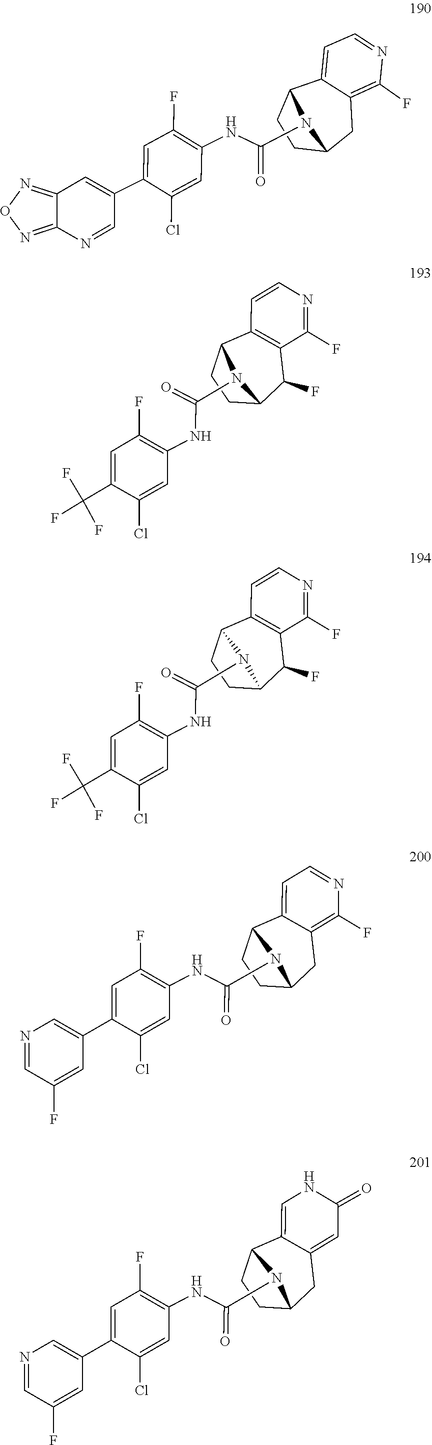 [SMILES: O=C(Nc1cc(Cl)c(-c2cnc3nonc3c2)cc1F)N1[C@H]2CC[C@@H]1c1ccnc(F)c1C2 ; O=C(Nc1cc(Cl)c(C(F)(F)F)cc1F)N1[C@@H]2CC[C@H]1[C@H](F)c1c2ccnc1F ; O=C(Nc1cc(Cl)c(C(F)(F)F)cc1F)N1[C@@H]2CC[C@H]1c1ccnc(F)c1[C@H]2F ; O=C(Nc1cc(Cl)c(-c2cncc(F)c2)cc1F)N1[C@H]2CC[C@@H]1c1ccnc(F)c1C2 ; O=C(Nc1cc(Cl)c(-c2cncc(F)c2)cc1F)N1[C@H]2CC[C@@H]1c1c[nH]c(=O)cc1C2] 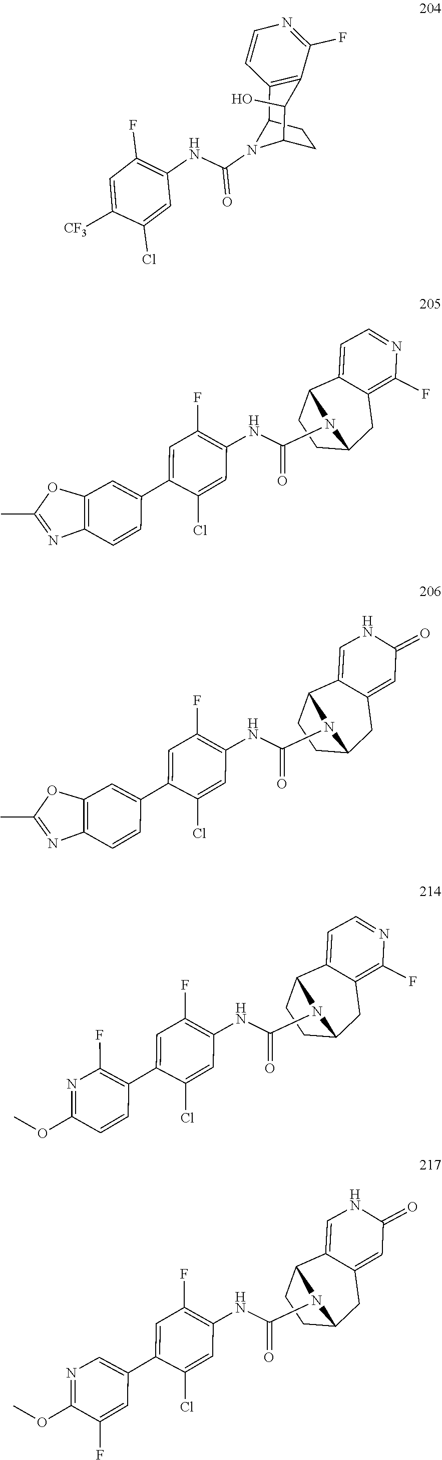 [SMILES: O=C(Nc1cc(Cl)c(C(F)(F)F)cc1F)N1C2CCC1C(O)c1c2ccnc1F ; Cc1nc2ccc(-c3cc(F)c(NC(=O)N4[C@H]5CC[C@@H]4c4ccnc(F)c4C5)cc3Cl)cc2o1 ; Cc1nc2ccc(-c3cc(F)c(NC(=O)N4[C@H]5CC[C@@H]4c4c[nH]c(=O)cc4C5)cc3Cl)cc2o1 ; COc1ccc(-c2cc(F)c(NC(=O)N3[C@H]4CC[C@@H]3c3ccnc(F)c3C4)cc2Cl)c(F)n1 ; COc1ncc(-c2cc(F)c(NC(=O)N3[C@H]4CC[C@@H]3c3c[nH]c(=O)cc3C4)cc2Cl)cc1F] 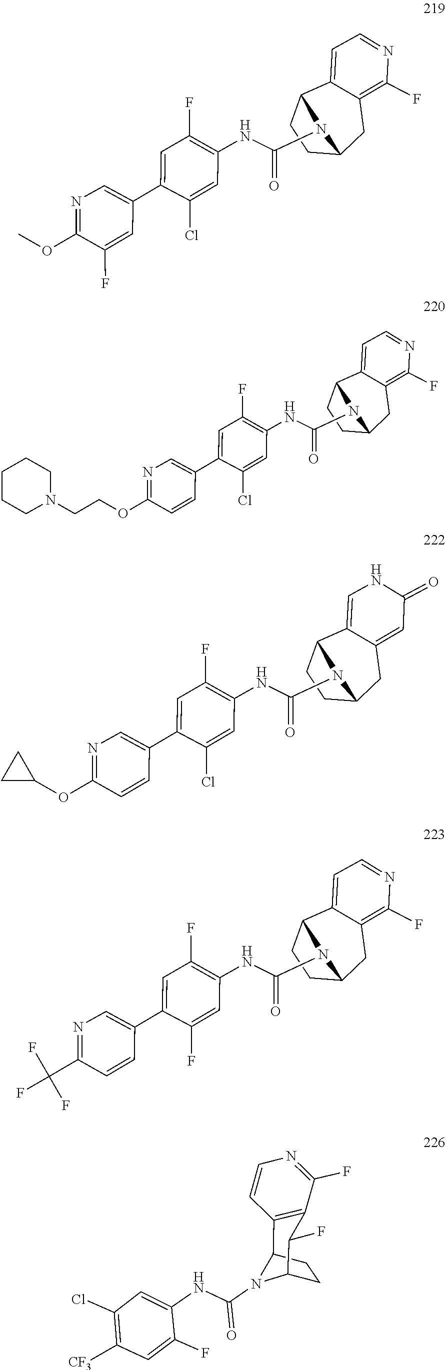 [SMILES: COc1ncc(-c2cc(F)c(NC(=O)N3[C@H]4CC[C@@H]3c3ccnc(F)c3C4)cc2Cl)cc1F ; O=C(Nc1cc(Cl)c(-c2ccc(OCCN3CCCCC3)nc2)cc1F)N1[C@H]2CC[C@@H]1c1ccnc(F)c1C2 ; O=C(Nc1cc(Cl)c(-c2ccc(OC3CC3)nc2)cc1F)N1[C@H]2CC[C@@H]1c1c[nH]c(=O)cc1C2 ; O=C(Nc1cc(F)c(-c2ccc(C(F)(F)F)nc2)cc1F)N1[C@H]2CC[C@@H]1c1ccnc(F)c1C2 ; O=C(Nc1cc(Cl)c(C(F)(F)F)cc1F)N1C2CCC1C(F)c1c2ccnc1F] 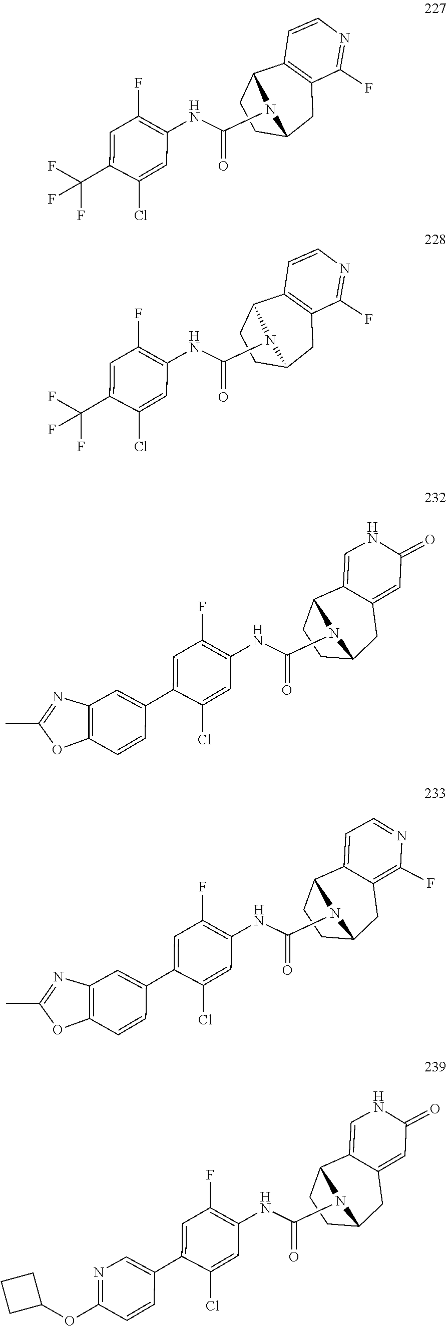 [SMILES: O=C(Nc1cc(Cl)c(C(F)(F)F)cc1F)N1[C@H]2CC[C@@H]1c1ccnc(F)c1C2 ; O=C(Nc1cc(Cl)c(C(F)(F)F)cc1F)N1[C@@H]2CC[C@H]1c1ccnc(F)c1C2 ; Cc1nc2cc(-c3cc(F)c(NC(=O)N4[C@H]5CC[C@@H]4c4c[nH]c(=O)cc4C5)cc3Cl)ccc2o1 ; Cc1nc2cc(-c3cc(F)c(NC(=O)N4[C@H]5CC[C@@H]4c4ccnc(F)c4C5)cc3Cl)ccc2o1 ; O=C(Nc1cc(Cl)c(-c2ccc(OC3CCC3)nc2)cc1F)N1[C@H]2CC[C@@H]1c1c[nH]c(=O)cc1C2] 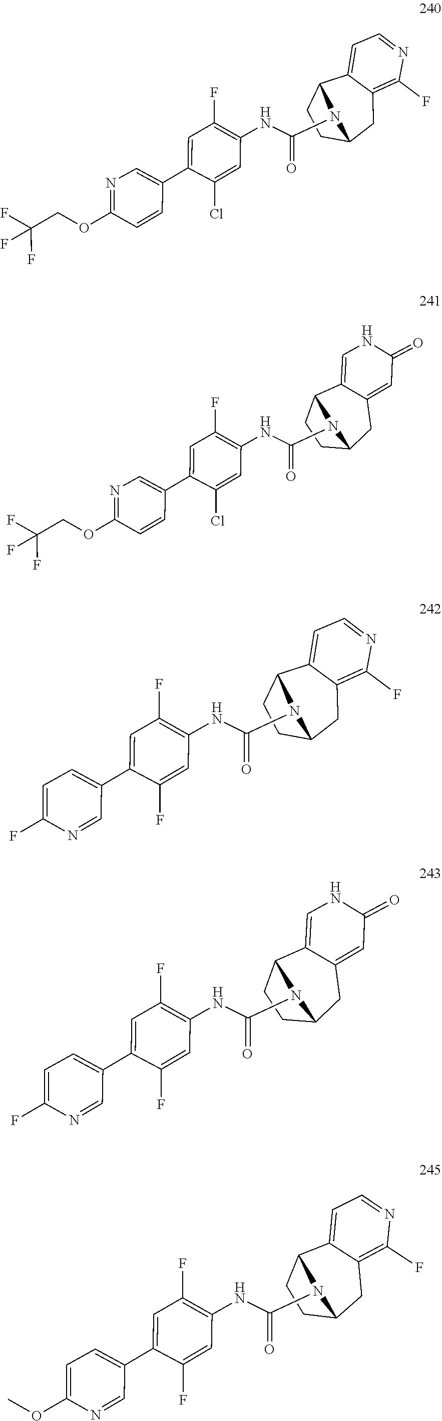 [SMILES: O=C(Nc1cc(Cl)c(-c2ccc(OCC(F)(F)F)nc2)cc1F)N1[C@H]2CC[C@@H]1c1ccnc(F)c1C2 ; O=C(Nc1cc(Cl)c(-c2ccc(OCC(F)(F)F)nc2)cc1F)N1[C@H]2CC[C@@H]1c1c[nH]c(=O)cc1C2 ; O=C(Nc1cc(F)c(-c2ccc(F)nc2)cc1F)N1[C@H]2CC[C@@H]1c1ccnc(F)c1C2 ; O=C(Nc1cc(F)c(-c2ccc(F)nc2)cc1F)N1[C@H]2CC[C@@H]1c1c[nH]c(=O)cc1C2 ; COc1ccc(-c2cc(F)c(NC(=O)N3[C@H]4CC[C@@H]3c3ccnc(F)c3C4)cc2F)cn1] 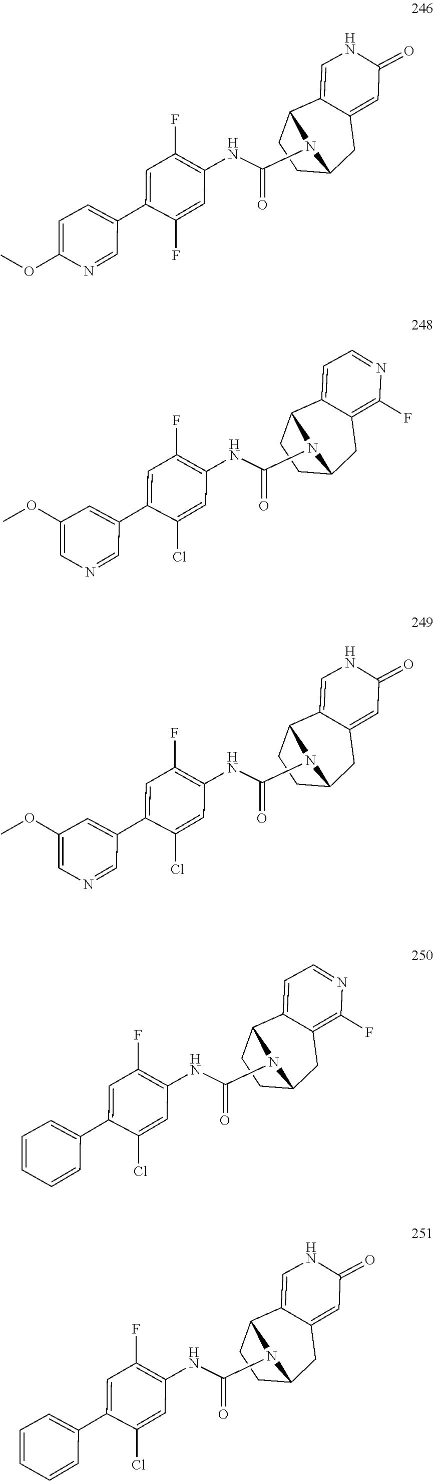 [SMILES: COc1ccc(-c2cc(F)c(NC(=O)N3[C@H]4CC[C@@H]3c3c[nH]c(=O)cc3C4)cc2F)cn1 ; COc1cncc(-c2cc(F)c(NC(=O)N3[C@H]4CC[C@@H]3c3ccnc(F)c3C4)cc2Cl)c1 ; COc1cncc(-c2cc(F)c(NC(=O)N3[C@H]4CC[C@@H]3c3c[nH]c(=O)cc3C4)cc2Cl)c1 ; O=C(Nc1cc(Cl)c(-c2ccccc2)cc1F)N1[C@H]2CC[C@@H]1c1ccnc(F)c1C2 ; O=C(Nc1cc(Cl)c(-c2ccccc2)cc1F)N1[C@H]2CC[C@@H]1c1c[nH]c(=O)cc1C2] 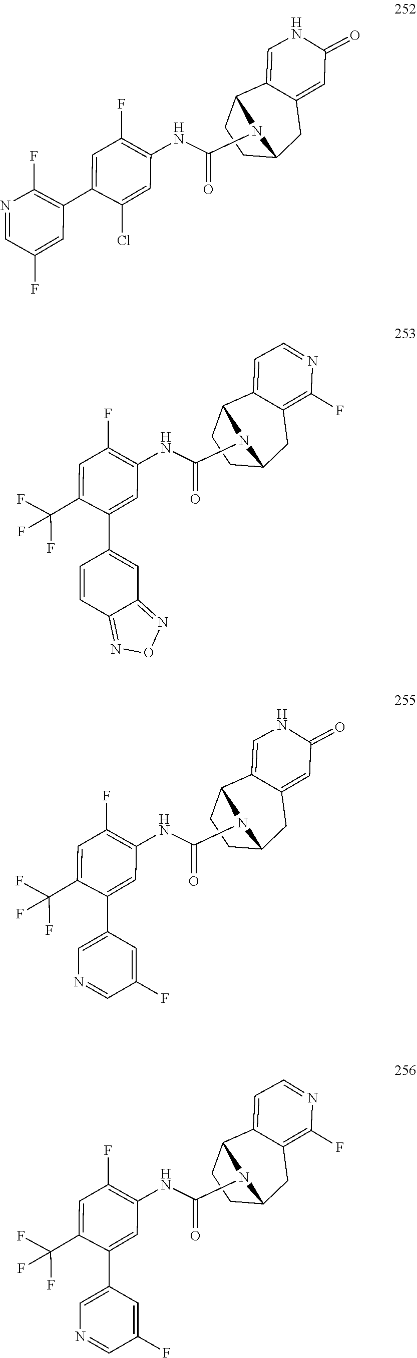 [SMILES: O=C(Nc1cc(Cl)c(-c2cc(F)cnc2F)cc1F)N1[C@H]2CC[C@@H]1c1c[nH]c(=O)cc1C2 ; O=C(Nc1cc(-c2ccc3nonc3c2)c(C(F)(F)F)cc1F)N1[C@H]2CC[C@@H]1c1ccnc(F)c1C2 ; O=C(Nc1cc(-c2cncc(F)c2)c(C(F)(F)F)cc1F)N1[C@H]2CC[C@@H]1c1c[nH]c(=O)cc1C2 ; O=C(Nc1cc(-c2cncc(F)c2)c(C(F)(F)F)cc1F)N1[C@H]2CC[C@@H]1c1ccnc(F)c1C2] 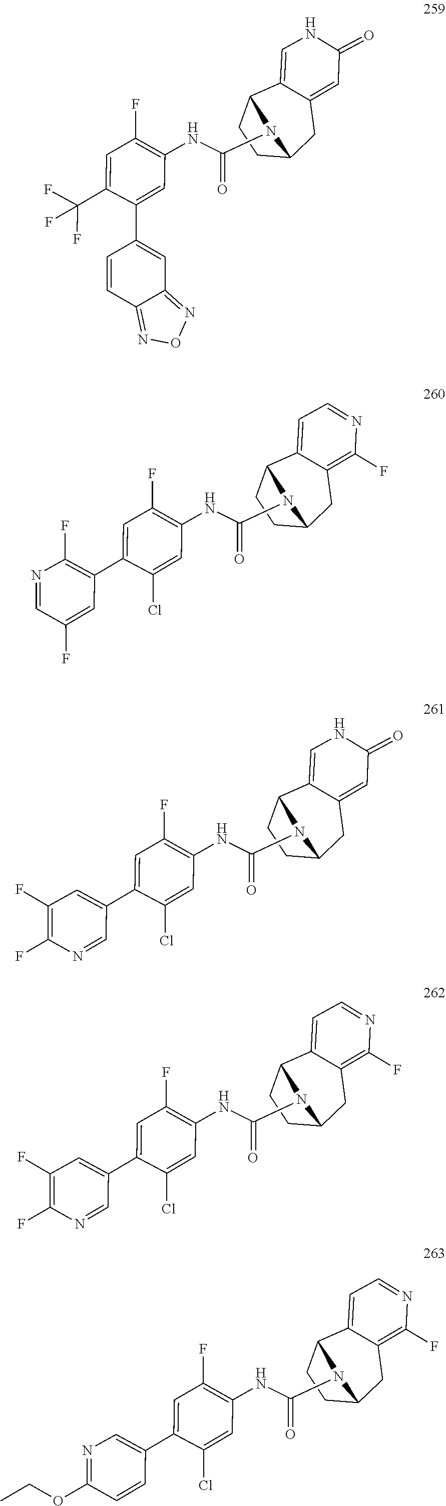 [SMILES: O=C(Nc1cc(-c2ccc3nonc3c2)c(C(F)(F)F)cc1F)N1[C@H]2CC[C@@H]1c1c[nH]c(=O)cc1C2 ; O=C(Nc1cc(Cl)c(-c2cc(F)cnc2F)cc1F)N1[C@H]2CC[C@@H]1c1ccnc(F)c1C2 ; O=C(Nc1cc(Cl)c(-c2cnc(F)c(F)c2)cc1F)N1[C@H]2CC[C@@H]1c1c[nH]c(=O)cc1C2 ; O=C(Nc1cc(Cl)c(-c2cnc(F)c(F)c2)cc1F)N1[C@H]2CC[C@@H]1c1ccnc(F)c1C2 ; CCOc1ccc(-c2cc(F)c(NC(=O)N3[C@H]4CC[C@@H]3c3ccnc(F)c3C4)cc2Cl)cn1] 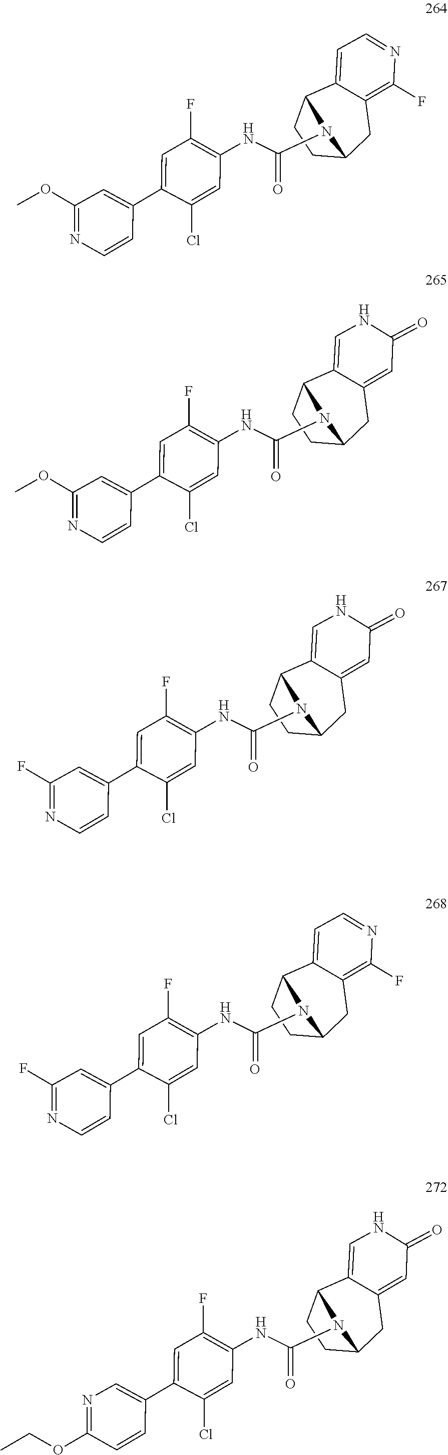 [SMILES: COc1cc(-c2cc(F)c(NC(=O)N3[C@H]4CC[C@@H]3c3ccnc(F)c3C4)cc2Cl)ccn1 ; COc1cc(-c2cc(F)c(NC(=O)N3[C@H]4CC[C@@H]3c3c[nH]c(=O)cc3C4)cc2Cl)ccn1 ; O=C(Nc1cc(Cl)c(-c2ccnc(F)c2)cc1F)N1[C@H]2CC[C@@H]1c1c[nH]c(=O)cc1C2 ; O=C(Nc1cc(Cl)c(-c2ccnc(F)c2)cc1F)N1[C@H]2CC[C@@H]1c1ccnc(F)c1C2 ; CCOc1ccc(-c2cc(F)c(NC(=O)N3[C@H]4CC[C@@H]3c3c[nH]c(=O)cc3C4)cc2Cl)cn1] 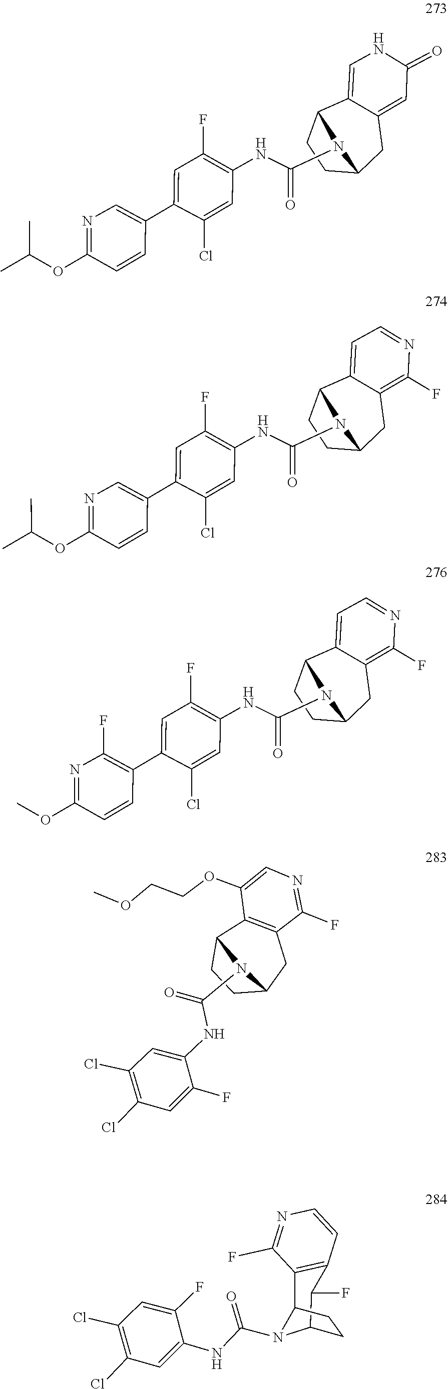 [SMILES: CC(C)Oc1ccc(-c2cc(F)c(NC(=O)N3[C@H]4CC[C@@H]3c3c[nH]c(=O)cc3C4)cc2Cl)cn1 ; CC(C)Oc1ccc(-c2cc(F)c(NC(=O)N3[C@H]4CC[C@@H]3c3ccnc(F)c3C4)cc2Cl)cn1 ; COc1ccc(-c2cc(F)c(NC(=O)N3[C@H]4CC[C@@H]3c3ccnc(F)c3C4)cc2Cl)c(F)n1 ; COCCOc1cnc(F)c2c1[C@H]1CC[C@@H](C2)N1C(=O)Nc1cc(Cl)c(Cl)cc1F ; O=C(Nc1cc(Cl)c(Cl)cc1F)N1C2CCC1C(F)c1ccnc(F)c12] 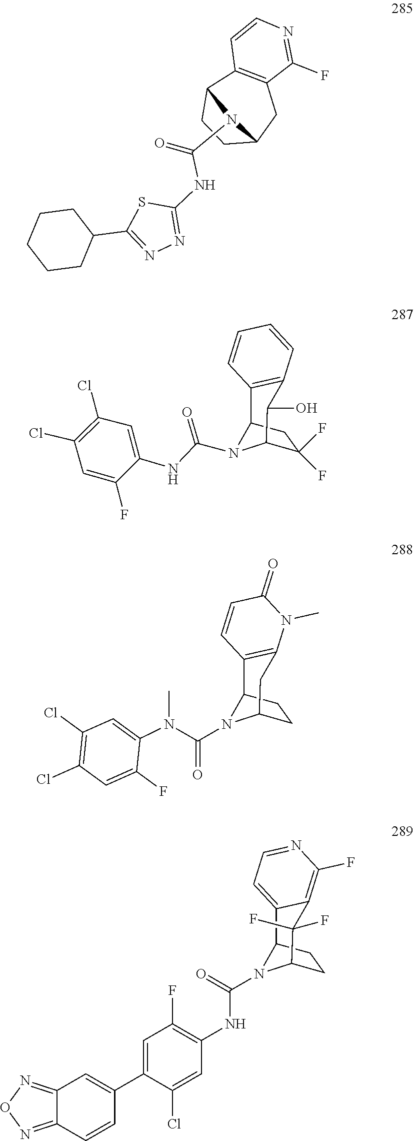 [SMILES: O=C(Nc1nnc(C2CCCCC2)s1)N1[C@H]2CC[C@@H]1c1ccnc(F)c1C2 ; O=C(Nc1cc(Cl)c(Cl)cc1F)N1C2CC(F)(F)C1C(O)c1ccccc12 ; CN(C(=O)N1C2CCC1c1ccc(=O)n(C)c1C2)c1cc(Cl)c(Cl)cc1F ; O=C(Nc1cc(Cl)c(-c2ccc3nonc3c2)cc1F)N1C2CCC1C(F)(F)c1c2ccnc1F] 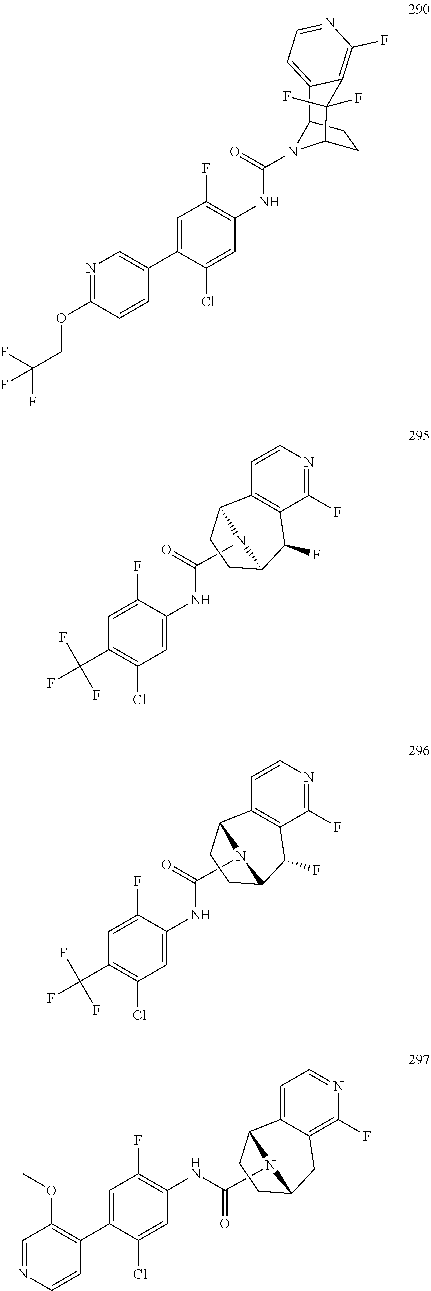 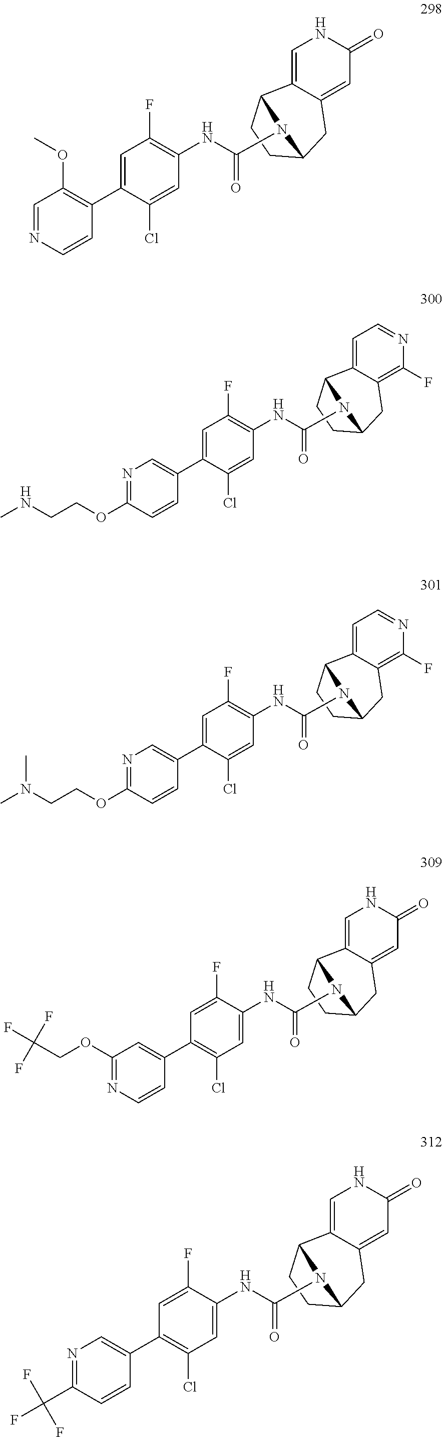 [SMILES: COc1cnccc1-c1cc(F)c(NC(=O)N2[C@H]3CC[C@@H]2c2c[nH]c(=O)cc2C3)cc1Cl ; CNCCOc1ccc(-c2cc(F)c(NC(=O)N3[C@H]4CC[C@@H]3c3ccnc(F)c3C4)cc2Cl)cn1 ; CN(C)CCOc1ccc(-c2cc(F)c(NC(=O)N3[C@H]4CC[C@@H]3c3ccnc(F)c3C4)cc2Cl)cn1 ; O=C(Nc1cc(Cl)c(-c2ccnc(OCC(F)(F)F)c2)cc1F)N1[C@H]2CC[C@@H]1c1c[nH]c(=O)cc1C2 ; O=C(Nc1cc(Cl)c(-c2ccc(C(F)(F)F)nc2)cc1F)N1[C@H]2CC[C@@H]1c1c[nH]c(=O)cc1C2] 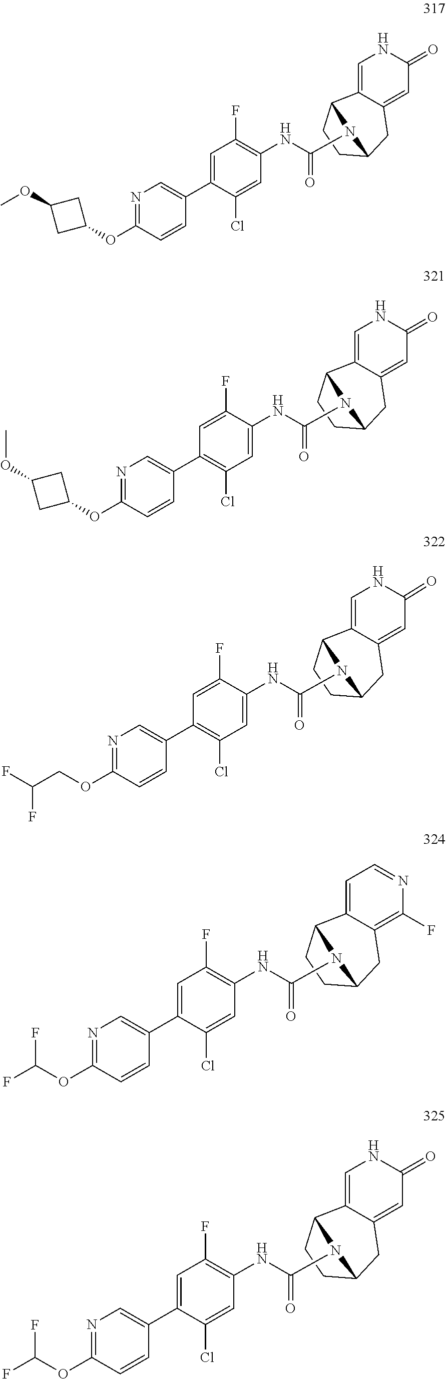 [SMILES: CO[C@H]1C[C@H](Oc2ccc(-c3cc(F)c(NC(=O)N4[C@H]5CC[C@@H]4c4c[nH]c(=O)cc4C5)cc3Cl)cn2)C1 ; CO[C@H]1C[C@@H](Oc2ccc(-c3cc(F)c(NC(=O)N4[C@H]5CC[C@@H]4c4c[nH]c(=O)cc4C5)cc3Cl)cn2)C1 ; O=C(Nc1cc(Cl)c(-c2ccc(OCC(F)F)nc2)cc1F)N1[C@H]2CC[C@@H]1c1c[nH]c(=O)cc1C2 ; O=C(Nc1cc(Cl)c(-c2ccc(OC(F)F)nc2)cc1F)N1[C@H]2CC[C@@H]1c1ccnc(F)c1C2 ; O=C(Nc1cc(Cl)c(-c2ccc(OC(F)F)nc2)cc1F)N1[C@H]2CC[C@@H]1c1c[nH]c(=O)cc1C2] 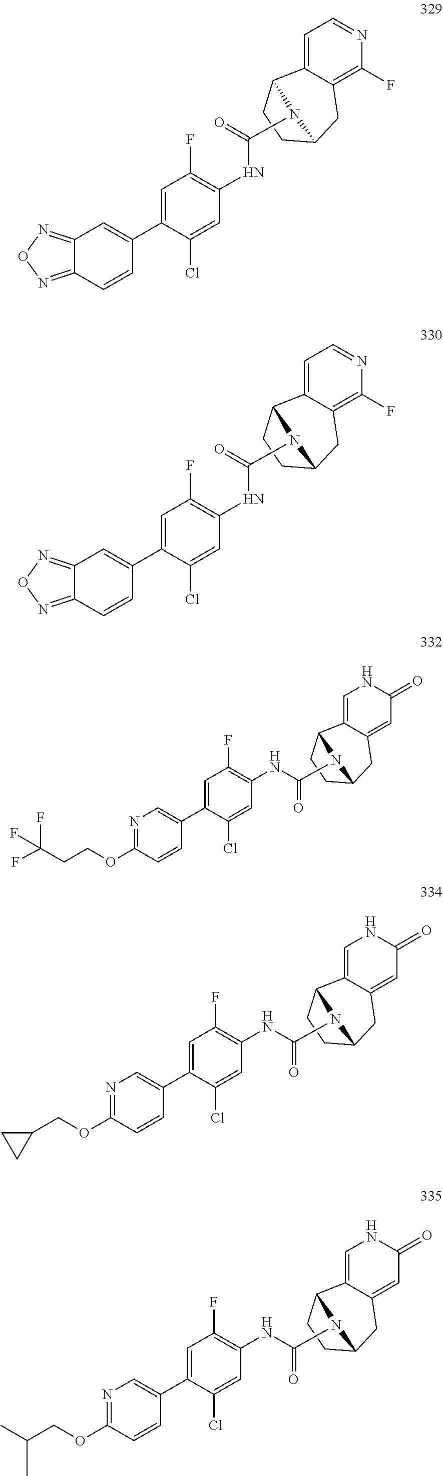 [SMILES: O=C(Nc1cc(Cl)c(-c2ccc3nonc3c2)cc1F)N1[C@@H]2CC[C@H]1c1ccnc(F)c1C2 ; O=C(Nc1cc(Cl)c(-c2ccc3nonc3c2)cc1F)N1[C@H]2CC[C@@H]1c1ccnc(F)c1C2 ; O=C(Nc1cc(Cl)c(-c2ccc(OCCC(F)(F)F)nc2)cc1F)N1[C@H]2CC[C@@H]1c1c[nH]c(=O)cc1C2 ; O=C(Nc1cc(Cl)c(-c2ccc(OCC3CC3)nc2)cc1F)N1[C@H]2CC[C@@H]1c1c[nH]c(=O)cc1C2 ; CC(C)COc1ccc(-c2cc(F)c(NC(=O)N3[C@H]4CC[C@@H]3c3c[nH]c(=O)cc3C4)cc2Cl)cn1] 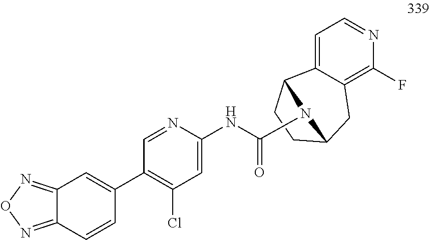 [SMILES: O=C(Nc1cc(Cl)c(-c2ccc3nonc3c2)cn1)N1[C@H]2CC[C@@H]1c1ccnc(F)c1C2]
and enantiomers thereof, and mixtures of enantiomers thereof, including racemic mixtures, and pharmaceutically acceptable salts and solvates thereof.

32. A pharmaceutical composition comprising a compound according to claim 1, and a pharmaceutically acceptable diluent, excipient, or carrier.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. A method of treating a disorder selected from the group consisting of a proliferative disorder, an immune disorder, asthma, chronic obstructive Pulmonary disease (COPD) and acute respiratory distress syndrome (ARDS), comprising administering to a subject a compound as defined in claim 1.

45. (canceled)

46. (canceled)

47. (canceled)
